(19) United States
(12) Patent Application Publication
(10) Pub. No.: US 2026/0184721 A2
(43) Pub. Date: Jul. 2, 2026

(54) FUSED RING COMPOUND, CONJUGATE THEREOF AND USE THEREOF

(71) Applicant: Bo Chen, Chengdu, Sichuan (CN)

(72) Inventor: Bo Chen, Sichuan (CN)

(73) Assignee: Keymed Biosciences (Chengdu) Co., Ltd., Chengdu, Sichuan (CN)

(21) Appl. No.: 19/131,820

(22) PCT Filed: Nov. 22, 2023
(86) PCT No.: PCT/CN2023/133434

§ 371 Date: Aug. 22, 2025

(30) Foreign Application Priority Data
Nov. 22, 2022 (CN) 202211545125.3
Jun. 9, 2023 (CN) 202310683602.0

(51) Int. Cl. C07D 491/147; A61K 31/4375; A61K 47/65; C07D 491/22

(52) U.S. Cl. CPC C07D 491/147; A61K 31/4375; A61K 47/65; C07D 491/22

(57) ABSTRACT

The present disclosure relates to a fused ring compound, a conjugate thereof and the use thereof. The compound and the conjugate of the present disclosure have good activity of inhibiting tumor cells, stability, and in vivo drug efficacy in animals.

The present application is a National Phase application filed under 35 USC § 371 of International Application No. PCT/CN2023/133434, filed Nov. 22, 2023, which claims the priority rights, and the benefit of the following prior applications: the Chinese invention patent application with application number 202211545125.3 and title “Fused ring compound, conjugate thereof and use thereof” filed with China National Intellectual Property Administration on Nov. 22, 2022, and the Chinese invention patent application with application number 202310683602.0 and title “Fused ring compound, conjugate thereof and use thereof” filed with China National Intellectual Property Administration on Jun. 9, 2023. The full text of the above-mentioned prior applications is incorporated herein by reference in their entirety.

### CO-FILED SEQUENCE LISTING

The present specification incorporates by reference in its entirety the sequence listing entitled “IEC240788PCT_Sequence listing.xml”, which was created on Feb. 19, 2025 and is 11 kilobytes in size, and filed electronically herewith. The entire contents of the Sequence Listing are incorporated herein by reference.

### TECHNICAL FIELD

The present disclosure relates to a fused ring compound, conjugate thereof and use thereof, belonging to the field of medicine.

### BACKGROUND

The concept of antibody-drug conjugate (ADC) or ADC drug has a long history. As early as 1913, Nobel Prize winner Professor Paul Ehrlich first proposed the concept of “magic bullet”, which is to install a cytotoxic drug on a specific monoclonal antibody to achieve targeted killing of tumor cells. The ADC drugs currently on the market are based on this theory. By conjugating a cytotoxic drug to a monoclonal antibody, the monoclonal antibody is used as a carrier to efficiently deliver a small molecule cytotoxic drug to a target tumor cell in a targeted manner.

ADC drug uses a specific linker to connect an antibody and a small molecule cytotoxic drug, and its main components include antibody, linker and small molecule cytotoxic drug. After an ADC drug enters the blood, its antibody component can recognize a target and binds to a tumor cell that highly expresses a cell surface antigen. When the ADC-antigen complex enters the tumor cell through endocytosis, the complex is degraded by lysosome, and the cytotoxic load (drug) will be released, destroying DNA or preventing tumor cell division, thereby killing the tumor cell.

The first-generation ADC drugs emerged from an attempt in 1958 to use anti-mouse leukocyte immunoglobulin coupled with methotrexate to treat leukemia. Mylotarg is the first launched ADC drug. It was approved by the FDA in 2000 for the treatment of acute myeloid leukemia, but it was withdrawn from the market because Mylotarg caused severe and fatal liver damage and had no obvious survival benefit. The disadvantages of the first-generation ADC drugs include: their antibodies were murine, their cytotoxicity was not potent enough, and their targets were low-expressed.

The starting point for the development of the second-generation ADC drugs is Trastuzumab Emtansine, which is the first approved ADC drug targeting breast cancer. The most successful ADC drug in the field of hematological tumors is Brentuximab Vedotin, which mainly targets classical Hodgkin's lymphoma and anaplastic large cell lymphoma. The advantages of the second-generation ADC drugs include: diversity in the development of target antigens and humanized antibodies, and the disadvantages include: too low or too high drug loading rate, narrow therapeutic window and low effectiveness.

The typical representative of the third-generation ADC drugs is Enfortumab Vedotin, which is the second ADC drug targeting solid tumors. It is suitable for patients to whom PD-1/PD-L1 antibody treatment is ineffective, but is not sensitive to tubulin inhibitors.

At present, it is still necessary to develop a drug molecule and conjugate thereof with reasonable molecular structure design and improved pharmacodynamic activity.

### SUMMARY OF THE INVENTION

To solve the above technical problems, the present disclosure provides a conjugate represented by the following Formula (C), or a stereoisomer, racemate, tautomer, isotopologue, isotope-labeled compound, nitroxide or pharmaceutically acceptable salt thereof:

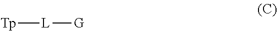

wherein, Tp represents a targeting moiety;
L is selected from a chemical bond or a linker;
G represents a group represented by the following Formula (G):

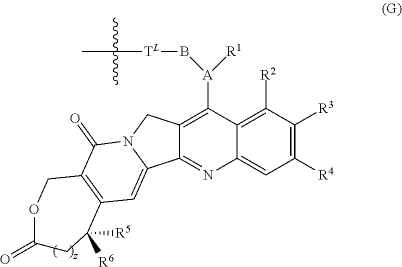

wherein, A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a ring structure which is unsubstituted or substituted with one, two or more R^{E}, for example, the ring structure is a 5- to 10-membered ring structure, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a ring structure which is unsubstituted or substituted with one, two or more R^{F}, for example, the ring structure is a 4- to 10-membered ring structure, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of alkyl, alkyloxy, halogen, hydroxyl, sulfhydryl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy, alkyl, alkyloxy, aryl, arylalkyl, aryloxy, heteroaryl, heteroarylalkyl, heteroaryloxy, amino;
B is absent or selected from the group consisting of triazolyl, —NR⁷— or —N(NR⁷R⁸)—;
R⁷ and R⁸ are the same or different and are independently selected from the group consisting of hydrogen and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B; each of R⁹, R¹⁰, R¹¹ and R¹² is the same or different, and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a ring structure which is unsubstituted or substituted with one, two or more R^{I}, for example, the ring structure is a 3- to 10-membered ring structure; or, R¹¹ and R¹², together with the atoms to which they are attached, form a ring structure which is unsubstituted or substituted with one, two or more R^{I}, for example, the ring structure is a 3- to 10-membered ring structure; or, R⁹ and R¹¹, together with the atoms to which they are attached, form a ring structure which is unsubstituted or substituted with one, two or more R^{I}, for example, the ring structure is a 3- to 10-membered ring structure; wherein any of the ring structures can be a monocyclic hydrocarbyl, bicyclic hydrocarbyl, heteromonocyclic hydrocarbyl, or heterobicyclic hydrocarbyl, each of which is unsubstituted or substituted with one, two or more R^{I}; for example, any of the ring structures can be the following group which is unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
each m is the same or different, and is independently selected from an integer of 0 to 10;
each n is the same or different, and is independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, aryloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
the wavy line represents the site attached to L;
provided that, when R⁶ is selected from the group consisting of alkyl, alkyloxy, aryl, arylalkyl, aryloxy, heteroaryl, heteroarylalkyl, heteroaryloxy or amino, each of which is unsubstituted or substituted with one, two or more R^{G}:
A is N; R¹ and R², together with the atoms to which they are attached, do not form a 5- to 10-membered ring structure which is unsubstituted or substituted with one, two or more R^{E}; and T^{L} is not a chemical bond when R⁷ is hydrogen;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbonyl; and R^{A} is selected from the following groups which are unsubstituted or substituted with one, two or more R^{C}: cycloalkyl, cycloalkylalkyl, cycloalkyloxy;
or, B is —N(NR⁷R⁸)—;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl.

According to the embodiments of the present invention, definitions of the groups in Formula (G) are independently selected from:
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, or heterobicyclic hydrocarbyl;
or R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, or heterobicyclic hydrocarbyl;
R⁴ is selected from the group consisting of alkyl, alkyloxy, halogen, hydroxyl, sulfhydryl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from the group consisting of triazolyl, —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached with L, and * represents the site attached with B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and are independently selected from the following groups which are unsubstituted or substituted with one, two or more R^{J}: alkyl, cycloalkyl, cycloalkylalkyl, aryl, arylalkyl alkyl, heteroaryl, heteroarylalkyl, heterocyclyl, heterocycloalkyl;
m and n are the same or different and are independently selected from an integer of 0 to 10;
each of R^{B}, R^{C}, R^{D}, R^{E}, R^{F}, R^{G}, R^{H}, R^{I} and R^{J} is the same or different and are independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different, and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and are independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—.

According to the embodiments of the present invention, definitions of the groups in Formula (G) may be independently selected from:
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxy alkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from alkyl, alkyloxy, halogen, hydroxyl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
m and n are the same or different, and are independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different, and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—.

According to the embodiments of the present invention, in the Formula (G) described in the context, the hydrogen, carbon or other atoms in the formula are optionally replaced by its isotope, regardless of whether the group is defined as being selected from the above definitions or directly indicated in the formula, and regardless of whether the group is defined as a substituted group or an unsubstituted group. For example, the hydrogen in any group of the formula (G) can be selected from ¹H, ²H or ³H. As an example, any hydrogen in the substituent of the unsubstituted or substituted alkyl, alkylene, alkylidene, phenyl, pyridyl or lactone group or on the ring-forming atoms thereof (if any) in the compound of formula (G) can be independently selected from ¹H, ²H or ³H.

According to the embodiments of the present invention, definitions of the groups in Formula (G) may be independently selected from:
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)ₙH—;
or, R^{A} and R¹, together with the atom to which they are attached, form a 3- to 8-membered ring structure which is unsubstituted or substituted with one, two or more R^{D}, for example, the 3- to 8-membered ring structure is 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino and cyano;
R⁵ is selected from hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy; preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and are independently selected from the groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and are independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to the embodiments of the present invention, definitions of the groups in Formula (G) may be independently selected from:
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
or, R^{A} and R¹, together with the atom to which they are attached, form a 3- to 8-membered ring structure which is unsubstituted or substituted with one, two or more R^{D}, for example, the 3- to 8-membered ring structure is a 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
provided that, R¹, R² and the atoms to which they are attached, or R², R³ and the atoms to which they are attached, at least one of these two sets of groups together form a 4- to 10-membered ring structure which is unsubstituted or substituted with one, two or more R^{E}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is selected from hydroxyl;
R⁶ is selected from the following groups which are unsubstituted or substituted with one, two or more R^{G}: C₁₋₁₀ alkyl, C₁₋₁₀ alkyloxy, C₆₋₁₀ aryl, C₆₋₁₀ arylalkyl, C₆₋₁₀ aryloxy, 5- to 6-membered heteroaryl, 5- to 6-membered heteroarylalkyl, 5- to 6-membered heteroaryloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein, any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, amino, nitro, the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃-6 cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different, and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to the embodiments of the present invention, definitions of the groups in Formula (G) may be independently selected from:
A is selected from N;
R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4,5-, 6-, 7-, 8-, 9- or 10-membered monocyclic hydrocarbyl, bicyclic hydrocarbyl, heteromonocyclic hydrocarbyl, heterobicyclic hydrocarbonyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is selected from hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy; preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is selected from —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano and nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to the embodiments of the present disclosure, R¹ and R², and the quinolyl to which they are connected form one of the following substructures, wherein the substructures may be unsubstituted or substituted with one, two or more R^{E}:

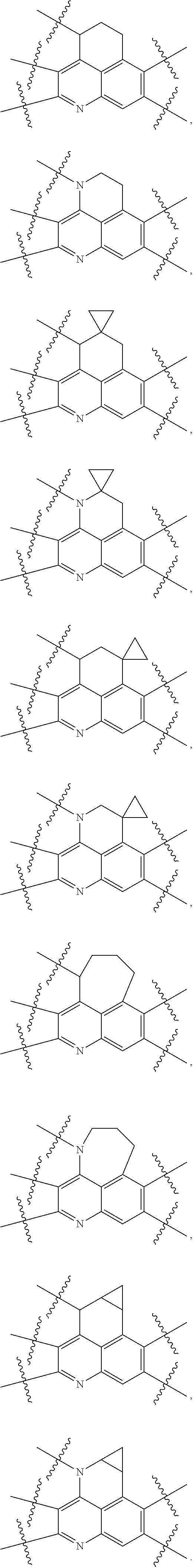

or, according to the embodiments of the present disclosure, R² and R³, together with the quinolyl to which they are connected, form one of the following substructures, wherein the substructures may be unsubstituted or substituted with one, two or more R^{F}:

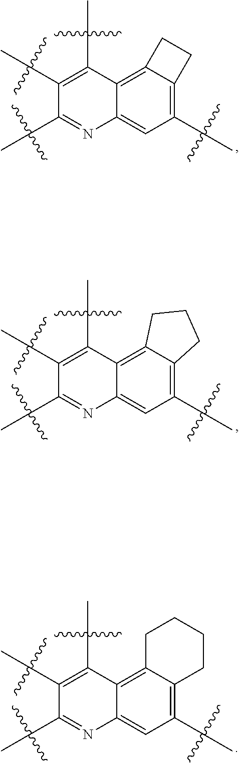 [SMILES: *c1cc2nc(*)c(*)c(*)c2c2c1CC2 ; *c1cc2nc(*)c(*)c(*)c2c2c1CCC2 ; *c1cc2nc(*)c(*)c(*)c2c2c1CCCC2]

According to the embodiments of the present disclosure, B is absent or selected from the group consisting of

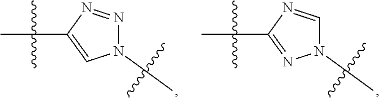 [SMILES: *c1cn(*)nn1 ; *c1ncn(*)n1]

—NH— or —N(NR⁷R⁸)—, wherein R⁷ and R⁸ independently have the definitions as described above.

According to the embodiments of the present disclosure, G is selected from the group represented by the following Formula (G-1):

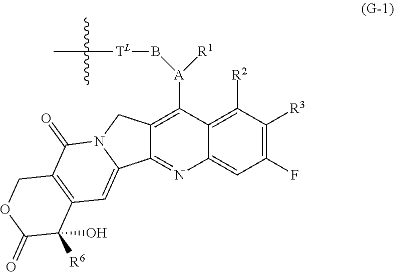

wherein, A, B, T^{L}, R¹, R², R³, and R⁶ have the definitions as described above.

According to the embodiments of the present disclosures, G is selected from the group represented by Formula (G-2) or (G-3):

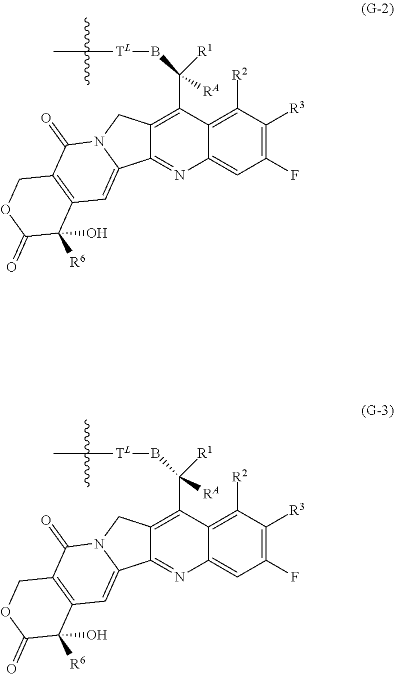

wherein, R^{A}, B, T^{L}, R¹, R², R³, and R⁶ have the definitions as described above.

According to the embodiments of the present disclosures, G is selected from the following group or the group formed by connecting -T^{L}- with the following group, at the wavy line of the following group:

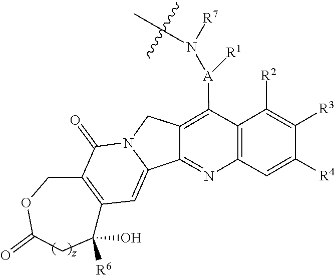

wherein, A, R¹, R², R³, R⁴, R⁶, R⁷, and z have the definitions as described above.

According to the embodiments of the present disclosures, G is selected from the following group or the group formed by connecting -T^{L}- with the following group, at the wavy line of the following group:

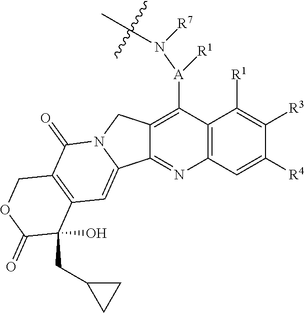

wherein, A, R¹, R², R³, R⁴, and R⁷ have the definitions as described above.

According to the embodiments of the present disclosures, G is selected from the following group or the group formed by connecting -T^{L}- with the following group, at the wavy line of the following group:

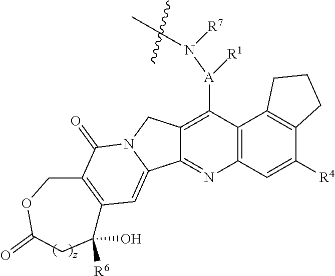

wherein, A, R¹, R⁴, R⁶, R⁷, and z have the definitions as described above.

According to the exemplary embodiments of the present disclosures, G is selected from the following groups or the groups formed by connecting -T^{L}- with the following group, at the wavy line of the following groups:

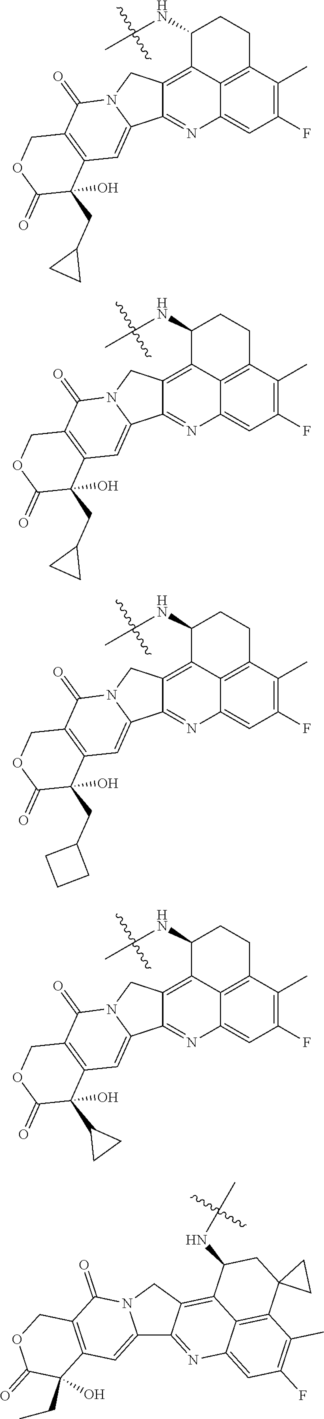 [SMILES: *N[C@@H]1CCc2c(C)c(F)cc3nc4c(c1c23)Cn1c-4cc2c(c1=O)COC(=O)[C@]2(O)CC1CC1 ; *N[C@H]1CCc2c(C)c(F)cc3nc4c(c1c23)Cn1c-4cc2c(c1=O)COC(=O)[C@]2(O)CC1CC1 ; *N[C@H]1CCc2c(C)c(F)cc3nc4c(c1c23)Cn1c-4cc2c(c1=O)COC(=O)[C@]2(O)CC1CCC1 ; *N[C@H]1CCc2c(C)c(F)cc3nc4c(c1c23)Cn1c-4cc2c(c1=O)COC(=O)[C@]2(O)C1CC1 ; *N[C@H]1CC2(CC2)c2c(C)c(F)cc3nc4c(c1c23)Cn1c-4cc2c(c1=O)COC(=O)[C@]2(O)CC]

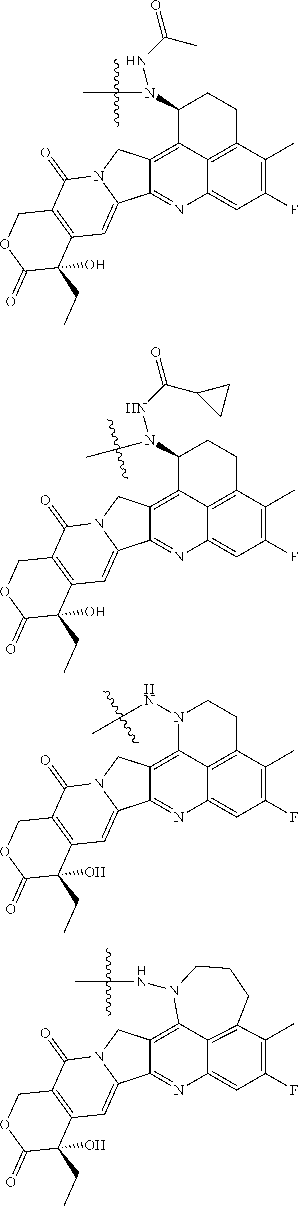 [SMILES: *N(NC(C)=O)[C@H]1CCc2c(C)c(F)cc3nc4c(c1c23)Cn1c-4cc2c(c1=O)COC(=O)[C@]2(O)CC ; *N(NC(=O)C1CC1)[C@H]1CCc2c(C)c(F)cc3nc4c(c1c23)Cn1c-4cc2c(c1=O)COC(=O)[C@]2(O)CC ; *NN1CCc2c(C)c(F)cc3nc4c(c1c23)Cn1c-4cc2c(c1=O)COC(=O)[C@]2(O)CC ; *NN1CCCc2c(C)c(F)cc3nc4c(c1c23)Cn1c-4cc2c(c1=O)COC(=O)[C@]2(O)CC]

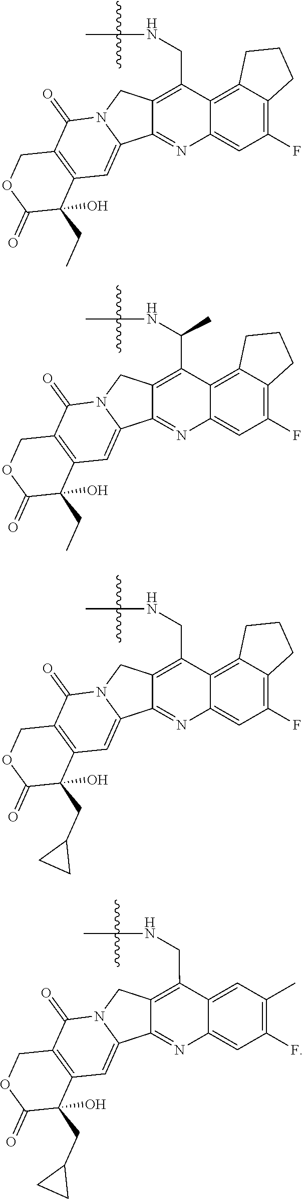 [SMILES: *NCc1c2c(nc3cc(F)c4c(c13)CCC4)-c1cc3c(c(=O)n1C2)COC(=O)[C@]3(O)CC ; *N[C@@H](C)c1c2c(nc3cc(F)c4c(c13)CCC4)-c1cc3c(c(=O)n1C2)COC(=O)[C@]3(O)CC ; *NCc1c2c(nc3cc(F)c4c(c13)CCC4)-c1cc3c(c(=O)n1C2)COC(=O)[C@]3(O)CC1CC1 ; *NCc1c2c(nc3cc(F)c(C)cc13)-c1cc3c(c(=O)n1C2)COC(=O)[C@]3(O)CC1CC1]

In the above aspect of the present invention, L is selected from a chemical bond or a linker represented by the Formula (L) as defined below:

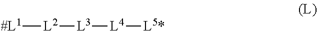

wherein, L¹ is a linking moiety to the targeting moiety Tp, formed from a reactive group L^{1′} through its coupling with the targeting moiety Tp, and # represents the site attached to the targeting moiety Tp; for example, L^{1′} is a maleimide group, then L¹ is the following structure:

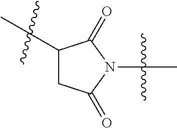 [SMILES: *C1CC(=O)N(*)C1=O]

or its ring-opening form:

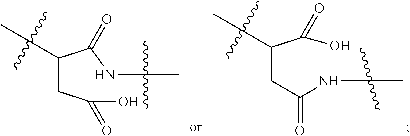 [SMILES: *NC(=O)C(*)CC(=O)O ; *NC(=O)CC(*)C(=O)O]

L² is absent or a spacer between L¹ and L³;
L³ is a peptide moiety;
L⁴ is absent or a spacer between the peptide moiety and L⁵;
L⁵ is a linking moiety between L⁴ and a bioactive molecule G, formed from a reactive group L^{5′} through its reaction with the bioactive molecule G or intermediates thereof, and * represents the site attached to the bioactive molecule G;
optionally:
the following hydrophilic moiety or steric hindrance moiety is inserted between the above moieties of L, preferably between L¹ and L², or between L² and L³, or is inserted by replacing L²:
hydrophilic moiety:

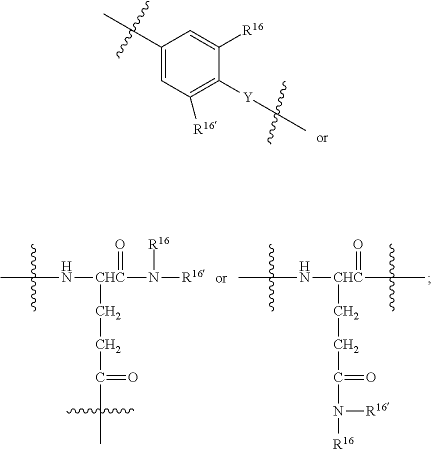

steric hindrance moiety:

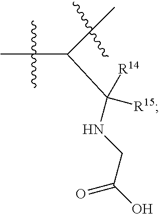

R¹⁶ and R^{16′} are the same or different, and at least one of them is selected from a hydrophilic group, and the other is selected from the following substituents: hydrogen, halogen, cyano, amino, nitro, and the following groups which are unsubstituted or substituted with one, two or more R^{zg}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, aminocarbonyl;
the hydrophilic group is selected from a polyethylene glycol group, a C₁₋₁₀ alkyl substituted with 1 to 10 hydroxyl groups, or a sugar ring-containing group, or C₁₋₆ alkyl-NHCO— (e.g., C₁₋₄ alkyl-NHCO—), preferably a polyethylene glycol group, more preferably —C(═O)—NH—(CH₂CH₂O)ₚ—C₁₋₁₀ alkyl or —NH—(CH₂CH₂O)ₚ—C₁₋₁₀ alkyl, or preferably a C₁₋₁₀ alkyl substituted with 1 to 10 hydroxyl groups, more preferably

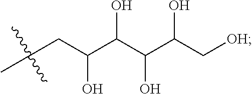 [SMILES: *CC(O)C(O)C(O)C(O)CO]

each p is the same or different, and is independently selected from an integer of 0 to 10, preferably 1, 2, 3, 4, 5, 6, 7 or 8;
Y is selected from the group consisting of O, S, C₁₋₁₀ alkylene, wherein 1, 2 or 3 of the methylene groups of the alkylene can be optionally replaced by O or S;
R¹⁴ and R¹⁵ are the same or different and are independently selected from the group consisting of hydrogen, halogen, cyano, amino, nitro, and the following groups which are unsubstituted or substituted with one, two or more R^{zh}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy;
or, R¹⁴ and R¹⁵, together with the atom to which they are attached, form a C₃₋₁₀ cycloalkyl which is unsubstituted or substituted with one, two or more R^{zh};
each of R^{zg} and R^{zh} is the same or different and is independently selected from the group consisting of halogen, hydroxyl, amino, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy;
in some embodiments of the present invention, the hydrophilic moiety or steric hindrance moiety in L as defined above is absent.

In some further preferred embodiments of the present invention, L¹ is formed from any of L^{1′} through its reaction with the targeting moiety Tp, L^{1′} is a sulfhydryl-reactive group, an amine-reactive group, a carboxyl-reactive group, a proline residue-reactive group, a tyrosine residue-reactive group, a disulfide bridge-containing group, etc.; for an antibody introduced with a non-natural amino acid, it can also be selected from reactive groups in click chemistry such as ketone, azide, alkyne, cyclopropene or diene;
L^{1′} is preferably a sulfhydryl-reactive group;
L^{1′} is further preferably a maleimide group or a substituted maleimide group, and L¹-L² preferably has the following structure:
a fragment that is prepared from (N-maleimidomethyl)-carboxylic acid-N-hydroxysuccinimide ester, and has the structure as follows:

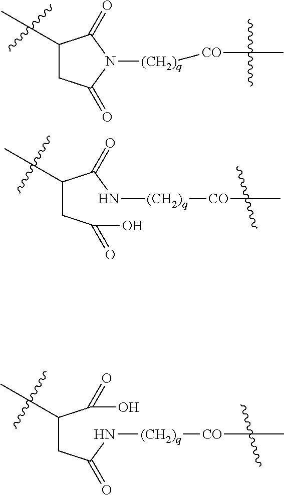

(q is an integer of 0 to 10, preferably 1, 2, 3, 4, 5, 6, 7 or 8);
or a fragment that is prepared from (meta)-maleimidobenzoic acid N-hydroxysuccinimide ester (MBS), and has the structure as follows:

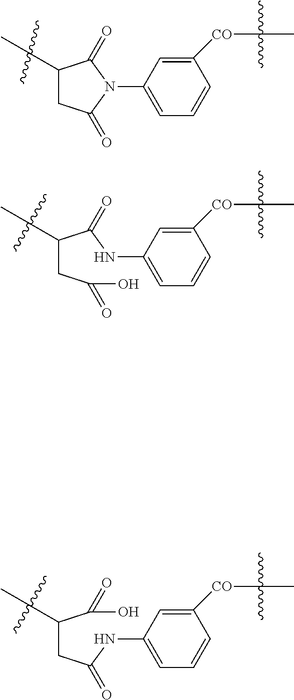 [SMILES: *C(=O)c1cccc(N2C(=O)CC(*)C2=O)c1 ; *C(=O)c1cccc(NC(=O)C(*)CC(=O)O)c1 ; *C(=O)c1cccc(NC(=O)CC(*)C(=O)O)c1]

or a fragment that is prepared from 4-(N-maleimidomethyl)-cyclohexane-1-carboxylic acid N-hydoxysuccinimide ester (SMCC), and has the structure as follows:

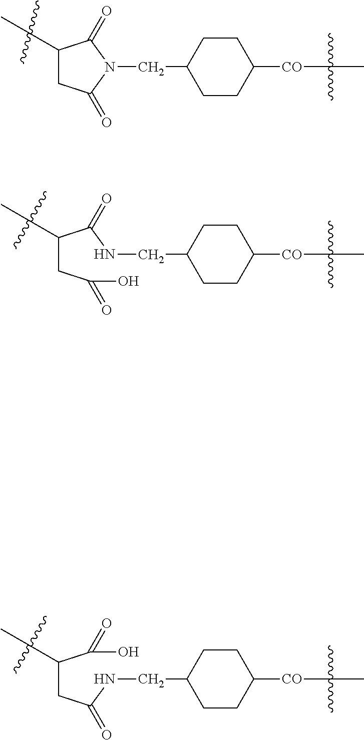 [SMILES: *C(=O)C1CCC(CN2C(=O)CC(*)C2=O)CC1 ; *C(=O)C1CCC(CNC(=O)C(*)CC(=O)O)CC1 ; *C(=O)C1CCC(CNC(=O)CC(*)C(=O)O)CC1]

or L^{1′} is preferably a sulfhydryl-reactive group having the following structure:
Hal-Het-
Hal is selected from the group consisting of halogen, OMs, OTs, OTf, nitro, and the following groups which are optionally substituted with one or more R^{z6}: alkyl thioether group, aryl thioether group, heteroaryl thioether group, alkyl sulfoxide group, aryl sulfoxide group, heteroaryl sulfoxide group, alkyl sulfonyl group, aryl sulfonyl group, heteroaryl sulfonyl group; wherein R^{z6} is independently selected from the group consisting of H (hydrogen), D (deuterium), halogen, CN, nitro, C₁₋₆ alkyl, halogenated C₁₋₆ alkyl, C₁₋₆ alkoxy, 6- to 10-membered aryl and 5- to 12-membered heteroaryl;
Het is selected from 5- to 10-membered heteroaryl which is optionally substituted with one or more R^{z7}; wherein R^{z7} is independently selected from the group consisting of H (hydrogen), D (deuterium), halogen, CN, nitro, C₁₋₄ alkyl and halogenated C₁₋₄ alkyl;
in a preferred embodiment, Hal is preferably mesyl, and Het is preferably pyrimidinyl;
in a preferred embodiment, Hal-Het- is:

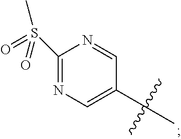 [SMILES: *c1cnc(S(C)(=O)=O)nc1]

the corresponding L¹ has the structure as follows:

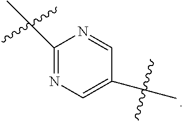 [SMILES: *c1cnc(*)nc1]

and L^{1′}-L² preferably has the structure as follows:

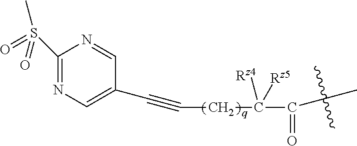

q is an integer selected from 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.
further preferably has the structure as follows:

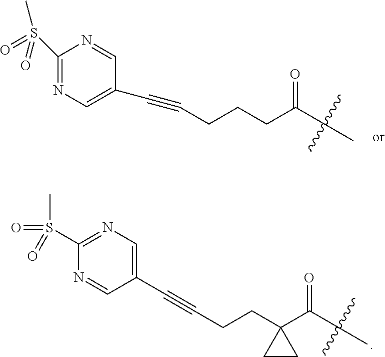 [SMILES: *C(=O)CCCC#Cc1cnc(S(C)(=O)=O)nc1 ; *C(=O)C1(CCC#Cc2cnc(S(C)(=O)=O)nc2)CC1]

In a further preferred embodiment of the present invention,
L² is absent or selected from the group consisting of C₁₋₁₀ alkylene, C₂₋₁₀ alkenylene, C₂₋₁₀ alkynylene, C₃₋₁₀ cycloalkyl, C₆₋₁₂ aryl or 6- to 12-membered heteroaryl or a combination of the above fragments, and is optionally interrupted by a carbonyl group, O, S, or N, and optionally substituted with C₁₋₆ alkyl, C₃₋₆ cycloalkyl, halogen, halogenated C₁₋₆ alkyl, and optionally, the alkyl or halogenated alkyl, together with the C atom to which it is connected, forms a C₃₋₆ cycloalkyl, L² is connected to L¹ or L³ through any functional group or covalent bond;
preferably, L² is connected to the N-terminal of the peptide moiety of L³ through —C(R^{z4}R^{z5})—CO—.

In a further preferred embodiment of the present invention, R^{z4} and R^{z5} are the same or different, and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.

In a further preferred embodiment of the present invention, L² is selected from the group consisting of —(CH₂)_{q}—, —(CH₂)_{q}—C(═O)—, or —(C≡C)—(CH₂)_{q}—C(R^{z4}R^{z5})—C(═O)—, wherein q is an integer of 0 to 10, preferably 1, 2, 3, 4, 5, 6, 7 or 8;
in a further preferred embodiment of the present invention, R^{z4} and R^{z5} are the same or different, and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.

In a further preferred embodiment of the present invention,
L³ is selected from a divalent peptide group comprising 2 to 8 optionally substituted natural amino acid residues or optionally substituted non-natural amino acid residues, each of the amino acid residues is the same or different and is independently selected from the following amino acid residues: alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucine (Nle), selenocysteine (Sec), pyrrolysine (Pyl), homoserine, homocysteine, demethylpyrrolysine.

Further preferably, L³ is selected from a divalent peptide group composed of 2, 3, 4, 5 or 6 optionally substituted natural amino acid residues or optionally substituted non-natural amino acid residues, each of the amino acid residues is the same or different and is independently selected from the following amino acid residues: alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucine (Nle), selenocysteine (Sec), pyrrolysine (Pyl), homoserine, homocysteine, demethylpyrrolysine; for example, -ValCit-; -CitVal-; -AlaAla-; -AlaCit-; -CitAla-; -AsnCit-; -CitAsn-; -CitCit-; -ValGlu-; -GluVal-; -SerCit-; -CitSer-; -LysCit-; -CitLys-; -AspCit-; -CitAsp-; -AlaVal-; -ValAla-; -PheAla-; -AlaPhe-; -PheLys-; -LysPhe-; -ValLys-; -LysVal-; -AlaLys-; -LysAla-; -PheCit-; -CitPhe-; -LeuCit-; -CitLeu-; -IleCit-; -CitIle-; -PheArg-; -ArgPhe-; -CitTrp-; -TrpCit-; -PhePheLys-; -LysPhePhe-; -DPhePheLys-; -DLysPhePhe-; -GlyPheLys-; -LysPheGly-; -GlyPheLeuGly-; -GlyLeuPheGly-; -AlaLeuAlaLeu-; -GlyGlyGly-; -GlyGlyGlyGly-; -GlyPheValGly-; -GlyValPheGly-; -GlyGlyPheGly-; -AlaAlaAla-; most preferably, L³ is -GlyGlyPheGly-.

Most preferably, L³ is -GlyGlyPheGly-.

In a further preferred embodiment of the present invention,
L⁴ is preferably a group with self-cleaving properties, and a self-cleaving group or self-immolative group is a group that initiates drug release through an intramolecular reaction such as 1,4-elimination, 1,6-elimination or cyclization elimination independent of the action of an enzyme.

L⁵ is formed from any reactive group L^{5′} through its reaction with a bioactive molecule or intermediate thereof, wherein L^{5′} is preferably a carboxylic acid group or an active ester group, which reacts with OH, SH or NH or NH₂ in the bioactive molecule to form L⁵ with the structure of: —C(O)O—, —C(O)S—, —C(O)N— or —C(O)NH— (including the atom of O, S or N in the bioactive molecule);
L⁴-L⁵ is preferably:
a PABC spacer arm with the structure as follows:

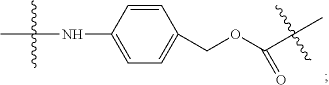 [SMILES: *Nc1ccc(COC(*)=O)cc1]

a GABA spacer arm with the structure as follows:

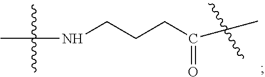 [SMILES: *NCCCC(*)=O]

an α,α-dimethyl GABA spacer arm with the structure as follows:

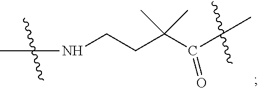 [SMILES: *NCCC(C)(C)C(*)=O]

or a β,β-dimethyl GABA spacer arm with the structure as follows:

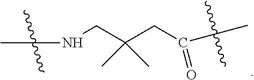 [SMILES: *NCC(C)(C)CC(*)=O]

Most preferably, L⁴-L⁵ is: —R¹—C(R²R)-T^{L}-
wherein:
R¹ is H or C₁₋₄ alkyl;
R^{z1} and R^{z3} are the same or different and are independently selected from H or C₁₋₄ alkyl;
T^{L} has the definition as described above, and one of T^{L} in L⁴-L⁵ or G is a chemical bond.

In the most preferred aspect of the present invention,
L¹ is generated by coupling Tp with a sulfhydryl-reactive group contained in maleimide group, a substituted maleimide group or Hal-Het-;
the following hydrophilic moiety is inserted between L¹ and L², or between L² and L³, or is inserted by replacing L²:
hydrophilic moiety:

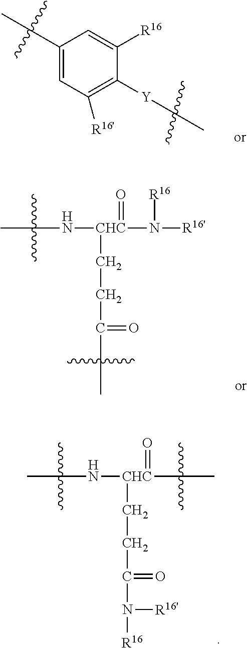

In the most preferred aspect of the present invention:
L¹ is generated by coupling Tp with a sulfhydryl-reactive group contained in maleimide group or a substituted maleimide group;
the following steric hindrance moiety is inserted between L¹ and L², or between L² and L³, or is inserted by replacing L²:
steric hindrance moiety:

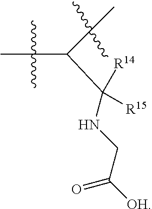

In the most preferred aspect of the present invention:
L¹ is generated by coupling Tp with a sulfhydryl-reactive group contained in Hal-Het-;
L⁴-L⁵ is: —NR^{z1}—C(R^{z2}R^{z3})-T^{L}-;
wherein:
R^{z1} is H or C₁₋₄ alkyl;
R^{z2} and R^{z3} are the same or different and are independently selected from H or C₁₋₄ alkyl;
T^{L} has the definition as described above, and one of T^{L} in L⁴-L⁵ or G is a chemical bond.

It is further preferred that:
when T^{L} in G is a chemical bond, L⁴-L⁵ is: —NR^{z1}—C(R^{z2}R^{z3})—O—(CH₂)ₘ—C(R^{z4}R^{z5})—CO—;
wherein:
R^{z1} is H or C₁₋₄ alkyl;
m is an integer of 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.

The present invention further provides a conjugate of the following structure:

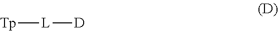

wherein Tp is the targeting moiety as defined above;
D is a bioactive molecular fragment, preferably a molecular fragment with anti-tumor biological activity;
wherein, L is selected from the linker represented by Formula (L):

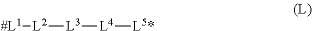

wherein, L¹ is a linking moiety to the targeting moiety Tp, and formed from a reactive group L^{1′} through its reaction with the targeting moiety Tp, and # represents the site connected to the Tp moiety;
L² is absent or a spacer between L¹ and L³;
L³ is a peptide moiety;
L⁴ is absent or a spacer between the peptide moiety and L⁵;
L⁵ is a linking moiety connecting L⁴ with the bioactive molecule D, and formed from a reactive group L⁵ through its reaction with the bioactive molecule or an intermediate thereof, and * represents the site connected to the bioactive molecule D;
provided that:
Condition I:
the following hydrophilic moiety is inserted between the fragments of L as defined above:
hydrophilic moiety:

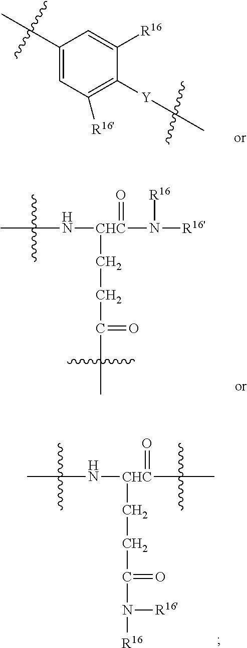

or Condition II:
L¹ is generated by coupling Tp with a sulfhydryl-reactive group contained in maleimide group or a substituted maleimide group;
the following steric hindrance moiety is inserted between L¹ and L², or between L² and L³, or inserted by replacing L²:
steric hindrance moiety:

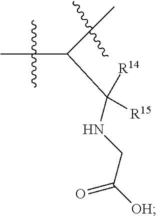

or Condition III:
L¹ is generated by coupling Tp with a sulfhydryl-reactive group contained in Hal-Het-;
L⁴-L⁵ is: —NR^{z1}—C(R^{z2}R^{z3})-T^{L}-;
wherein:
R^{z1} is H or C₁₋₄ alkyl;
R^{z2} and R^{z3} are the same or different, and are independently selected from H or C₁₋₄ alkyl;
T^{L} has the definition as described above.

In a further embodiment,
L¹ is generated by coupling Tp with a sulfhydryl-reactive group contained in maleimide group or Hal-Het-;
the following hydrophilic moiety is inserted between L¹ and L², or between L² and L³, or is inserted by replacing L²:
hydrophilic moiety:

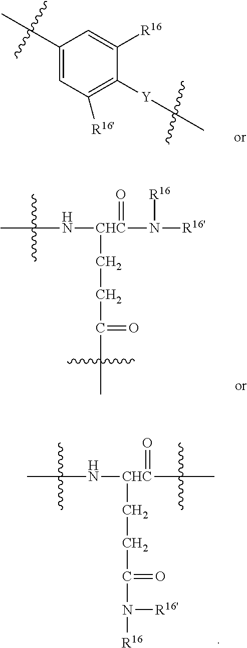

In a further embodiment,
in the most preferred aspect of the present invention:
L¹ is generated by coupling Tp with a sulfhydryl-reactive group contained in Hal-Het-;
L⁴-L⁵ is: —NR^{z1}—C(R^{z2}R^{z3})—O—(CH₂)ₘ—C(R^{z4}R^{z5})—CO—;
wherein:
R^{z1} is H or C₁₋₄ alkyl;
m is an integer of 0 to 4;
R^{z2} and R^{z3} are the same or different, and are independently selected from H or C₁₋₄ alkyl;
R^{z4} and R^{z5} are the same or different, and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.
or Condition IV:
L¹-L² is generated by coupling Tp with the following structure:

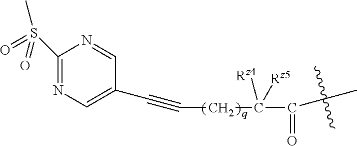

q is an integer selected from 0 to 10, preferably 1, 2, 3, 4, 5, 6, 7 or 8;
R^{z4} and R^{z5} are the same or different, and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
it is further preferred that R^{z4} and R^{z5} together form a C₃₋₆ cycloalkyl;
L¹-L² is most preferably generated by coupling Tp with

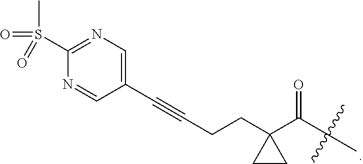 [SMILES: *C(=O)C1(CCC#Cc2cnc(S(C)(=O)=O)nc2)CC1]

The present invention also provides the following intermediate for the synthesis of the conjugate:

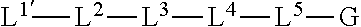

wherein L^{1′}, L², L³, L⁴, L⁵, G are as defined above.

The present invention also provides the following intermediate for the synthesis of the conjugate:

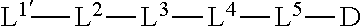

wherein L^{1′}, L², L³, L⁴, L⁵, and D are as defined above, provided that:
Condition I:
the following hydrophilic moiety is inserted between the fragments of L as defined above:
hydrophilic moiety:

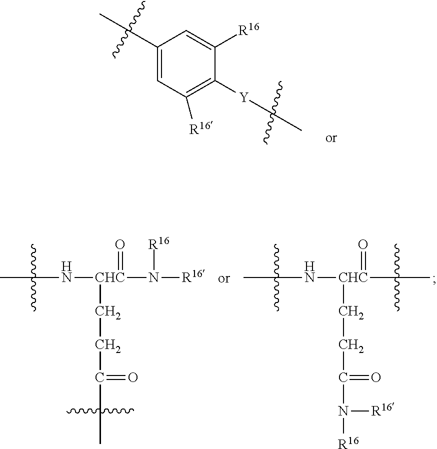

or Condition II:
L^{1′} is a sulfhydryl contained in maleimide group or a substituted maleimide group;
the following steric hindrance moiety is inserted between L¹ and L², or between L² and L³, or is inserted by replacing L²:
steric hindrance moiety:

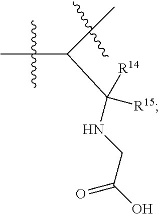

or Condition III:
L^{1′} is selected from the sulfhydryl-reactive group of Hal-Het-;
L⁴-L⁵ is: —NR—C(R^{z2}R^{z3})-T^{L}-;
wherein:
R^{z1} is H or C₁₋₄ alkyl;
R^{z2} and R^{z3} are the same or different and are independently selected from H or C₁₋₄ alkyl;
T^{L} is as defined above.
or Condition IV:
L^{1′}-L² has the following structure:

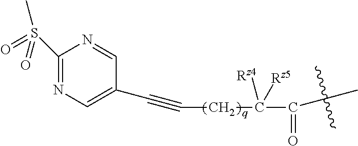

q is an integer of 0 to 10, preferably 1, 2, 3, 4, 5, 6, 7 or 8;
R^{z4} and R^{z5} are the same or different, and are independently selected from H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
further preferably, R^{z4} and R^{z5} together form a C₃₋₆ cycloalkyl;
L^{1′}-L² is most preferably

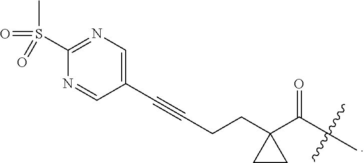 [SMILES: *C(=O)C1(CCC#Cc2cnc(S(C)(=O)=O)nc2)CC1]

In a further embodiment,
L^{1′} is a sulfhydryl contained in maleimide group, a substituted maleimide group or Hal-Het-;
the following hydrophilic moiety is inserted between L^{1′} and L², or between L² and L³, or is inserted by replacing L²:
hydrophilic moiety:

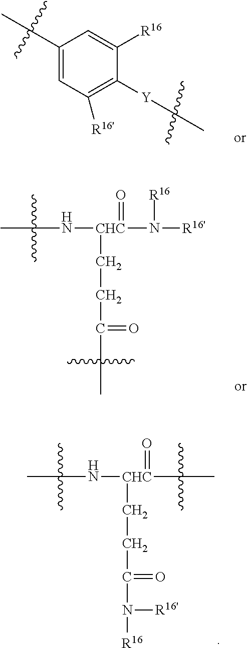

In a further embodiment,
in the most preferred aspect of the present invention:
L^{1′} is a sulfhydryl contained in Hal-Het-;
L⁴-L⁵ is: —NR^{z1}—C(R^{z2}R^{z3})—O—(CH₂)ₘ—C(R^{z4}R^{z5})—CO—;
wherein:
R¹ is H or C₁₋₄ alkyl;
m is an integer of 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl.

Those skilled in the art should understand that the bioactive molecule D can be a compound with biological activity or potential biological activity disclosed in the Chinese, American or European Pharmacopoeia or disclosed in other publications. As an example, the drug can be selected from cytotoxic drugs, cytostatic drugs or immunosuppressive drugs, such as anti-tubulin agents, tubulin inhibitors, DNA minor groove binders, DNA replication inhibitors, alkylating agents, antibiotics, antifolates, antimetabolites, chemosensitizers, topoisomerase inhibitors, vinca alkaloids, etc. Examples of such cytotoxic drugs include, for example, auristatin, camptothecin, duocarmycin, etoposide, maytansine and maytansine alkaloids, taxanes, benzodiazepines or benzodiazepines-containing drugs and vinca alkaloids.

Tp is a targeting moiety (e.g., a small molecule ligand, a protein, a polypeptide, a non-protein agent (e.g., sugar, RNA or DNA)).

In some preferred embodiments, the target of Tp is selected from the group consisting of epidermal growth factor, Trop-2, CD37, HER2, CD70, EGFRvIII, Mesothelin, Folate receptor1, Mucin 1, CD138, CD20, CD19, CD30, SLTRK6, Nectin 4, Tissue factor, Mucin16, Endothelin receptor, STEAP1, SLC39A6, Guanylylcyclase C, PSMA, CCD79b, CD22, Sodium phosphate cotransporter 2B, GPNMB, Trophoblast glycoprotein, AGS-16, EGFR, CD33, CD66e, CD74, CD56, PD-L1, TACSTD2, DR5, E16, 0772P, MPF, Napi3b, Sema 5b, PSCA hlg, ETBR, MSG783, STEAP2, TrpM4, CRIPTO, CD21, CD79b, FcRH2, NCA, MDP, IL20Rα, Brevican, E phB2R, ASLG659, PSCA, GEDA, BAFF-R, CD79a, CXCR5, HLA-DOB, P2X5, CD72, LY64, FcRH1, IRTA2, TENB2, integrin α5β6, α4β7, FGF2, FGFR2, HER3, CA6, DLL3, DLL4, P-cadherin, EpCAM, pCAD, CD223, LYPD3, LY6E, EFNA4, ROR1, SLITRK6, 5T4, ENPP3, Claudin18.2, BMPR1B, Tyro7, c-Met, ApoE, CD1 1c, CD40, CD45(PTPRC), CD49D(ITGA4), CD80, CSF1R, CTSD, GZMB, Ly86, MS4A7, PIK3AP1, PIK3CD, CCR5, IFNG, IL10RA1, I L-6, ACTA2, COL7A1, LOX, LRRC15, MCPT8, MMP10, NOG, SERPINE1, STAT1, TGFBR1, CTSS, PGF, VEGFA, C1QA, C₁QB, ANGPTL4, EGLN, EGLN3, BNIP3, AIF1, CCL5, CXCL10, CXCL11, IFI6, PLOD2, KISS1R, STC2, DDIT4, PFKFB3, PGK1, PDK1, AKR1C₁, AKR1C2, CADM1, CDH11, COL6A3, CTGF, HMOX1, KRT33A, LUM, WNT5A, IGFBP3, MMP14, CDCP1, PDGFRA, TCF4, TGF, TGFB1, TGFB2, CD1 1b, ADGRE1, EMR2, TNFRSF21, UPK1B, TNFSF9, MMP16, MFI2, IGF-1R, RNF43, NaPi2b and BCMA.

In some preferred embodiments, Tp is a small molecule ligand, such as a folic acid derivative, a glutamate urea derivative, a somatostatin derivative, an arylsulfonamide derivative (e.g., a carbonic anhydrase IX inhibitor), an ICG dye, a cyanine dye or a derivative thereof.

According to the embodiments of the present disclosure, the preferred ligand is selected from an antibody or antigen-binding fragment thereof, wherein the antibody is selected from a chimeric antibody, a humanized antibody or a fully human antibody; preferably a monoclonal antibody.

According to an exemplary embodiment of the present disclosure, the antibody or antigen-binding fragment thereof is at least one selected from the following antibodies or antigen-binding fragments thereof: anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-HER2 (ErbB2) antibody, anti-EGFR antibody, anti-B7-H3 antibody, anti-c-Met antibody, anti-HER3 (ErbB3) antibody, anti-HER4 (ErbB4) antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, and the antibody may be a bispecific antibody or a multispecific antibody.

As an example, the antibody or antigen-binding fragment thereof is at least one selected from the following antibodies or antigen-binding fragments thereof: Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96, and Glematumamab or Glembatumumab.

For example, Trastuzumab has sequences selected from the following:

light chain
SEQ ID NO: 1
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO: 2
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK

Pertuzumab has sequences selected from the following:

light chain
SEQ ID NO. 3
DIQMTQSPSSLSASVGDRVTITCKASQDVSIGVAWYQQKPGKAPK
LLIYSASYRYTGVPSRFSGSGSGTDFTLTISSLQPEDFATYYCQQ
YYIYPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO. 4
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK

Nimotuzumab has sequences selected from the following:

light chain
SEQ ID NO: 5
DIQMTQSPSSLSASVGDRVTITCRSSQNIVHSNGNTYLDWYQQTP
GKAPKLLIYKVSNRFSGVPSRFSGSGSGTDFTFTISSLQPEDIAT
YYCFQYSHVPWTFGQGTKLQITREVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
heavy chain
SEQ ID NO: 6
QVQLQQSGAEVKKPGSSVKVSCKASGYTFTNYYIYWVRQAPGQGL
EWIGGINPTSGGSNFNEKFKTRVTITVDESTNTAYMELSSLRSED
TAFYFCARQGLWFDSDGRGFDFWGQGSTVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK

Patritumab has sequences as the following:

light chain
SEQ ID NO: 7
DIEMTQSPDSLAVSLGERATINCRSSQSVLYSSSNRNYLAWYQQN
PGQPPKLLIYWASTRESGVPDRFSGSGSGTDFTLTISSLQAEDVA
VYYCQQYYSTPRTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
heavy chain
SEQ ID NO: 8
QVQLQQWGAGLLKPSETLSLTCAVYGGSFSGYYWSWIRQPPGKGL
EWIGEINHSGSTNYNPSLKSRVTISVETSKNQFSLKLSSVTAADT
AVYYCARDKWTWYFDLWGRGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKRVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK.

According to the embodiments of the present disclosure, the conjugate, or the linker or linker-drug thereof may be one selected from the followings, wherein u, v, and w are independently selected from an integer of 0 to 10 (e.g., 0, 1, 2, 3, 4, 5, 6, 7, 8, 9 or 10), G has the above-mentioned definition, LG has the above-mentioned definition of Tp; R²⁰, R^{20′} are the same or different, and are independently selected from the group consisting of H, C₁₋₄ alkyl, C₃₋₆ cycloalkyl, C₃₋₆ cycloalkyl-C₁₋₄ alkyl, or R²⁰, R^{20′} and the carbon atom to which they are attached together form a C₃₋₆ cycloalkyl;
for example, the C₁₋₄ alkyl can be independently selected from the group consisting of methyl, ethyl, propyl, isopropyl, butyl, isobutyl or tert-butyl; the C₃₋₆ cycloalkyl can be independently selected from the group consisting of cyclopropyl, cyclobutyl, cyclopentyl or cyclohexyl;
for example, both of R²⁰ and R^{20′} are H, or one is not H, or both are not H:

| No. | Conjugate, its linker or linker-drug |
|---|---|
| 1 | 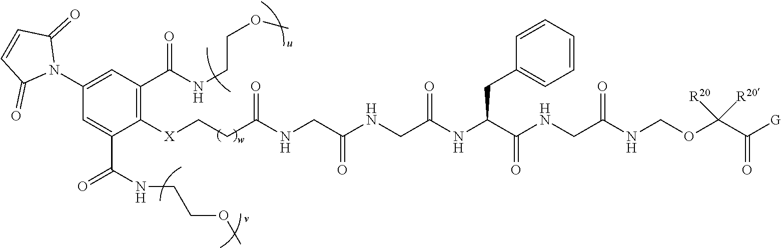 |
| 2 | 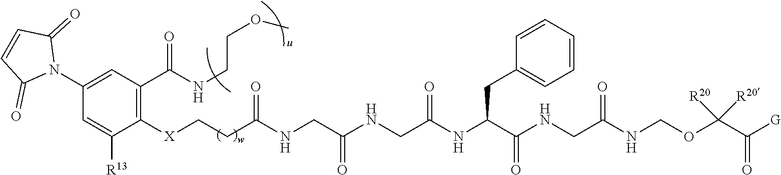 |
| 3 | 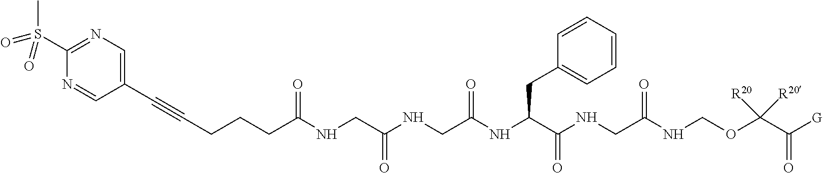 |
| 4 | 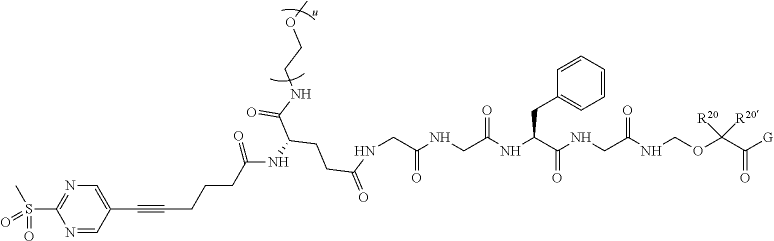 |
| 5 | 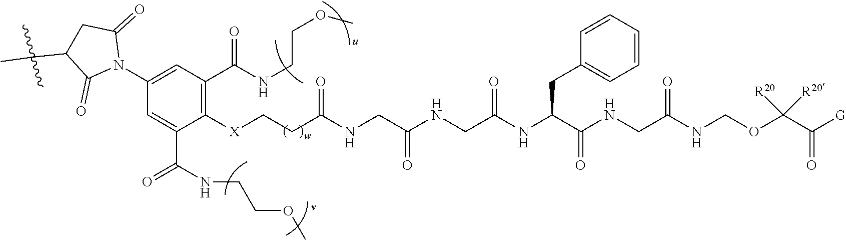 |
| 6 | 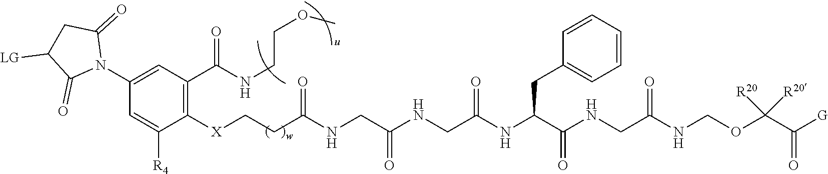 |
| 7 | 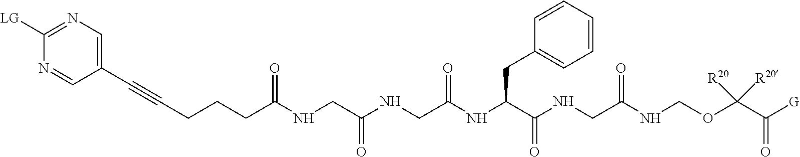 |
| 8 | 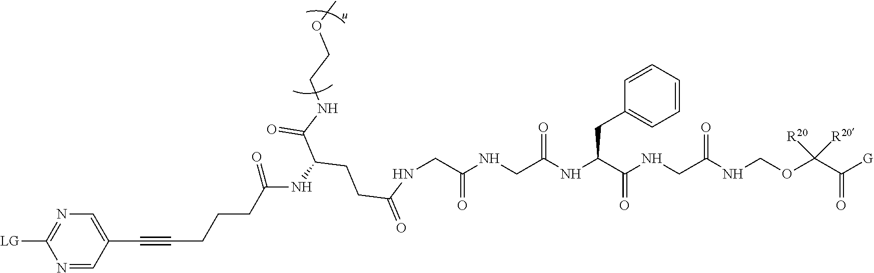 |
| 9 | 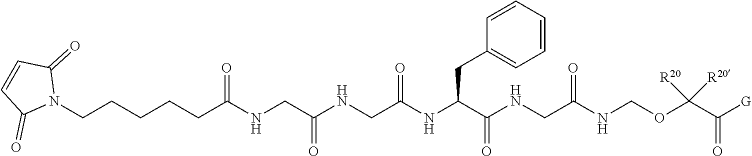 |
| 10 | 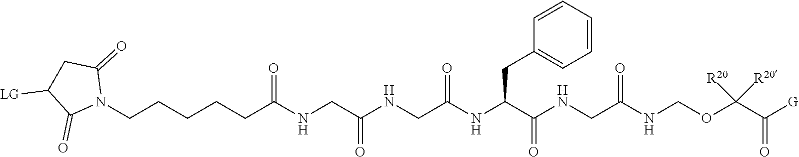 |
| 11 | 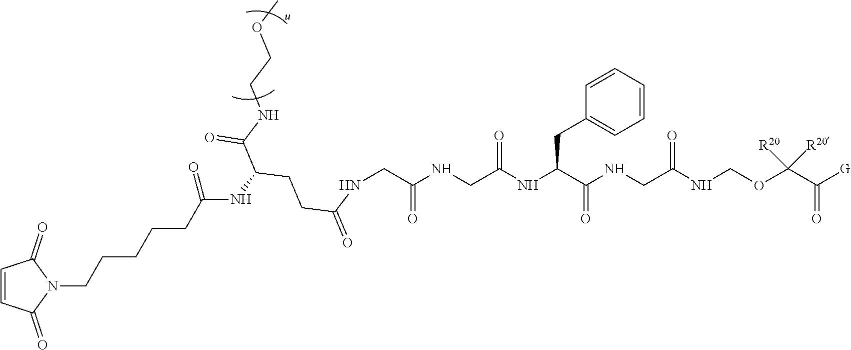 |
| 12 | 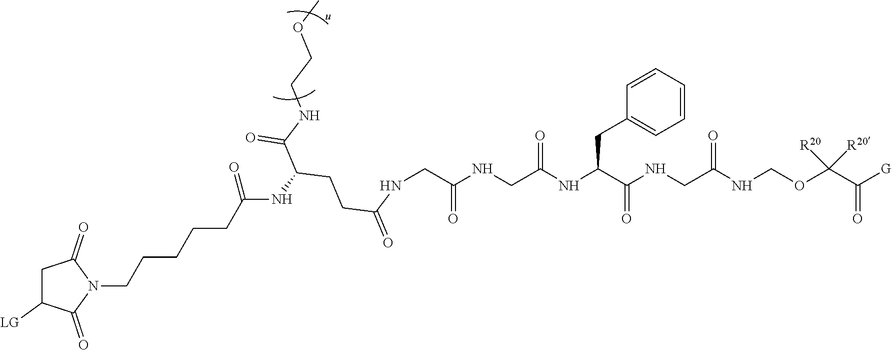 |
| 13 | 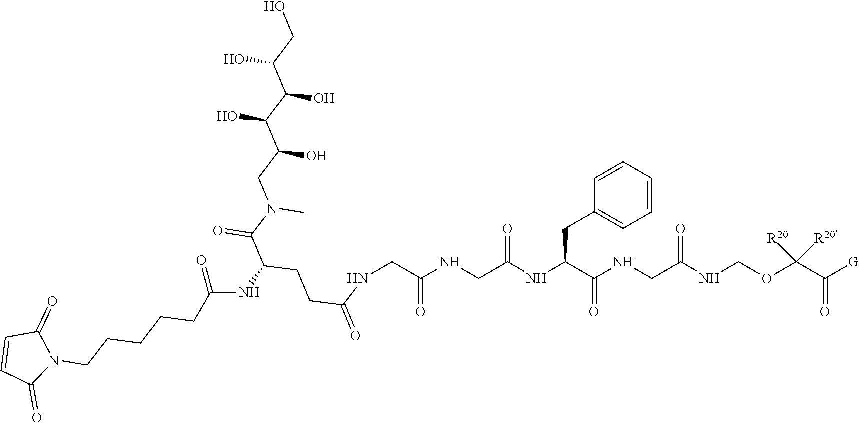 |
| 14 | 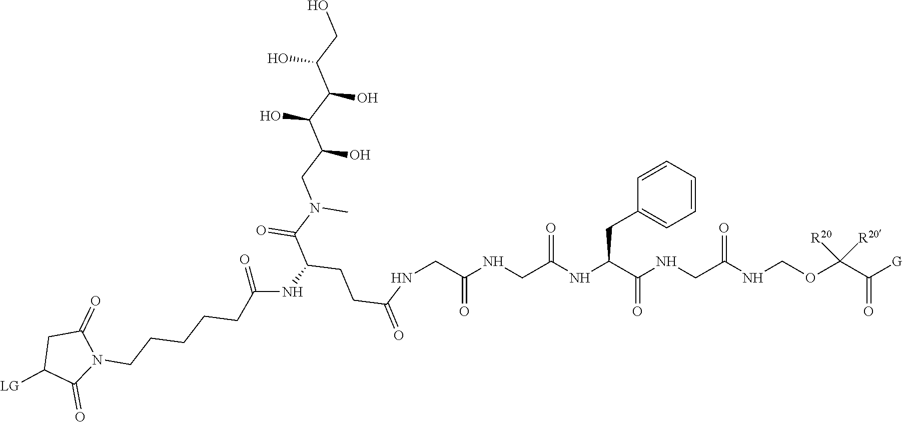 |
| 15 | 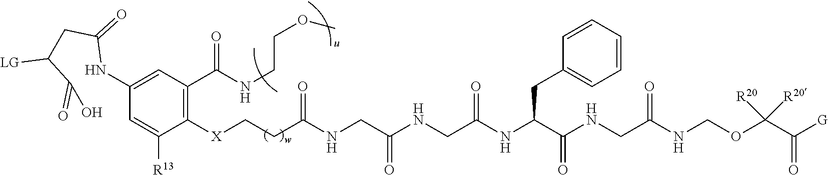 |
| 16 | 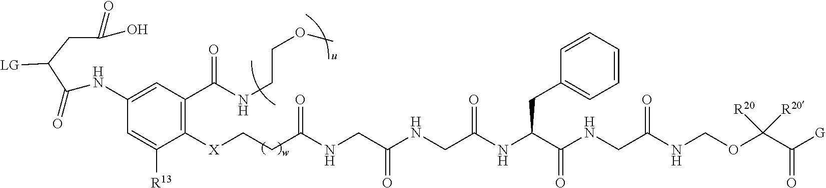 |
| 17 | 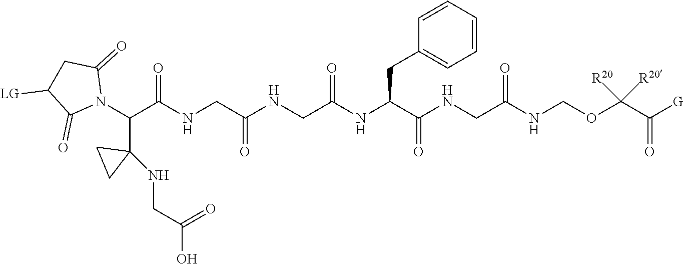 |
| 18 | 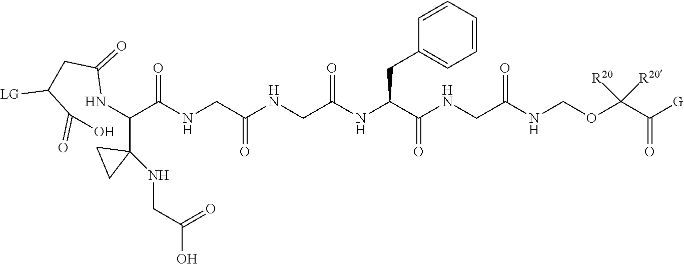 |
| 19 | 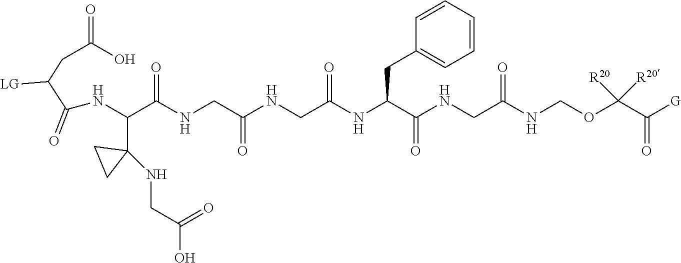 |
| 20 | 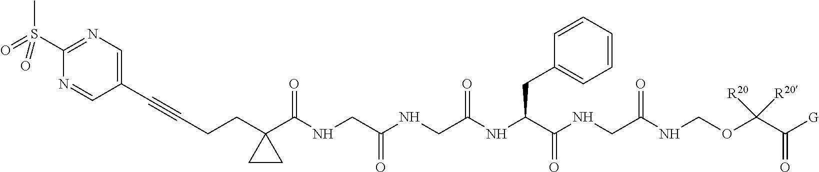 |
| 21 | 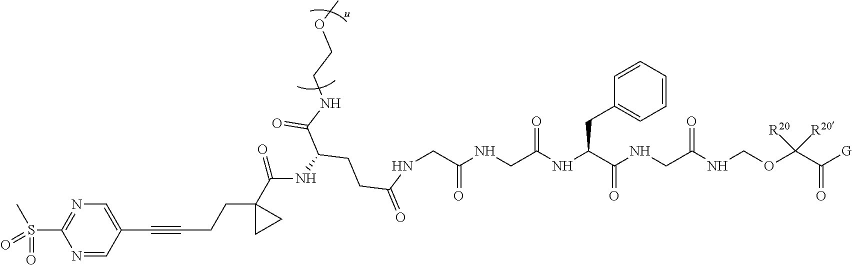 |
| 22 | 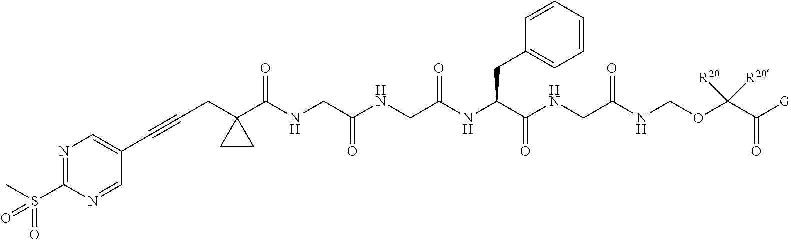 |
| 23 | 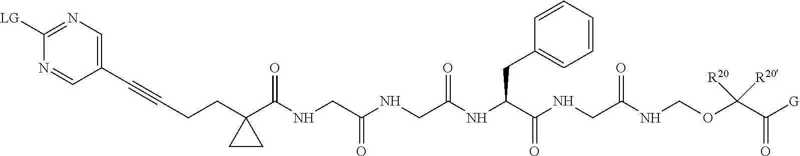 |
| 24 | 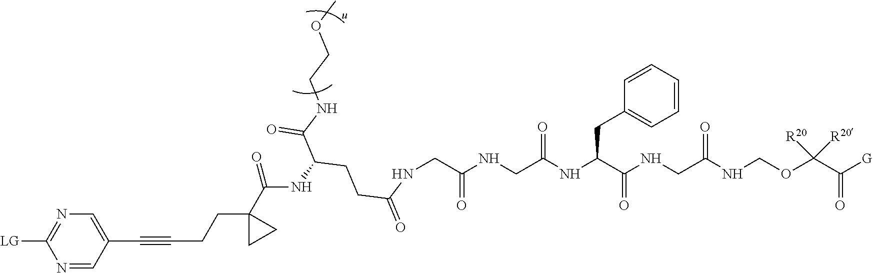 |
| 25 | 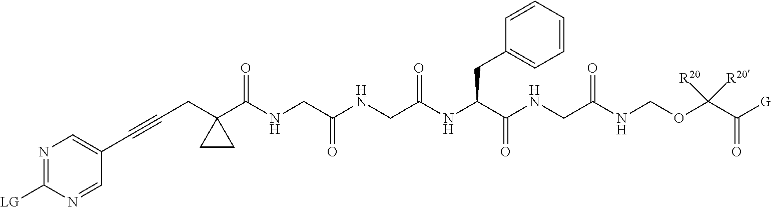 |
| 26 | 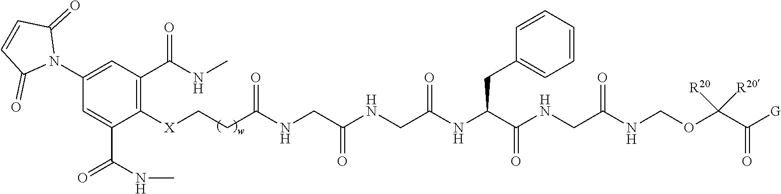 |
| 27 | 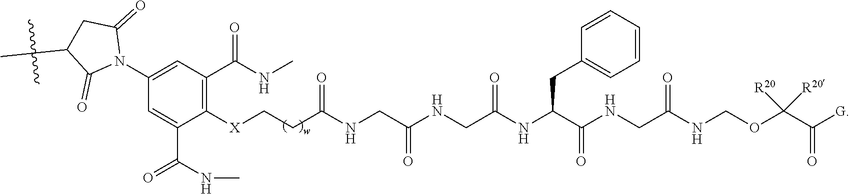 |

According to the embodiments of the present disclosure, the conjugate may have a structure as shown in the following formula:

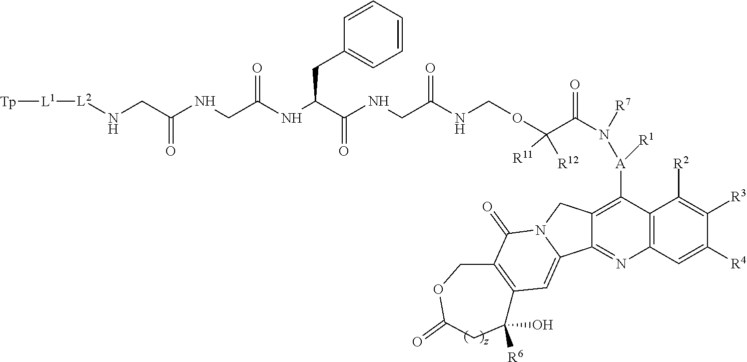

wherein, A, R¹, R², R³, R⁴, R⁶, R⁷, R¹¹, R¹², L¹, L², and Tp have the definitions described above.

According to the embodiments of the present disclosure, the conjugate may have a structure as shown in the following formula:

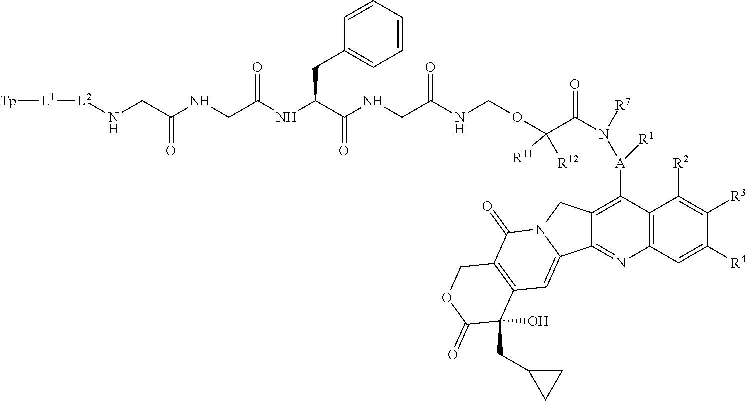

wherein, A, R¹, R², R³, R⁴, R⁷, R¹¹, R¹², L¹, L², and Tp have the definitions described above.

According to the embodiments of the present disclosure, the conjugate may have a structure as shown in the following formula:

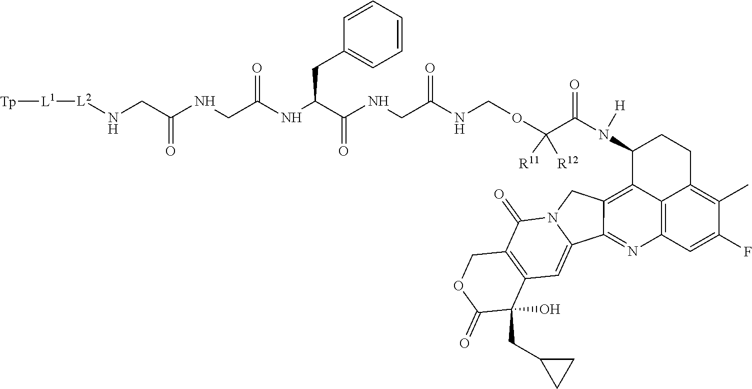

wherein, R¹¹, R¹², L¹, L², and Tp have the definitions described above.

According to the embodiments of the present disclosure, the conjugate may be one selected from the followings:

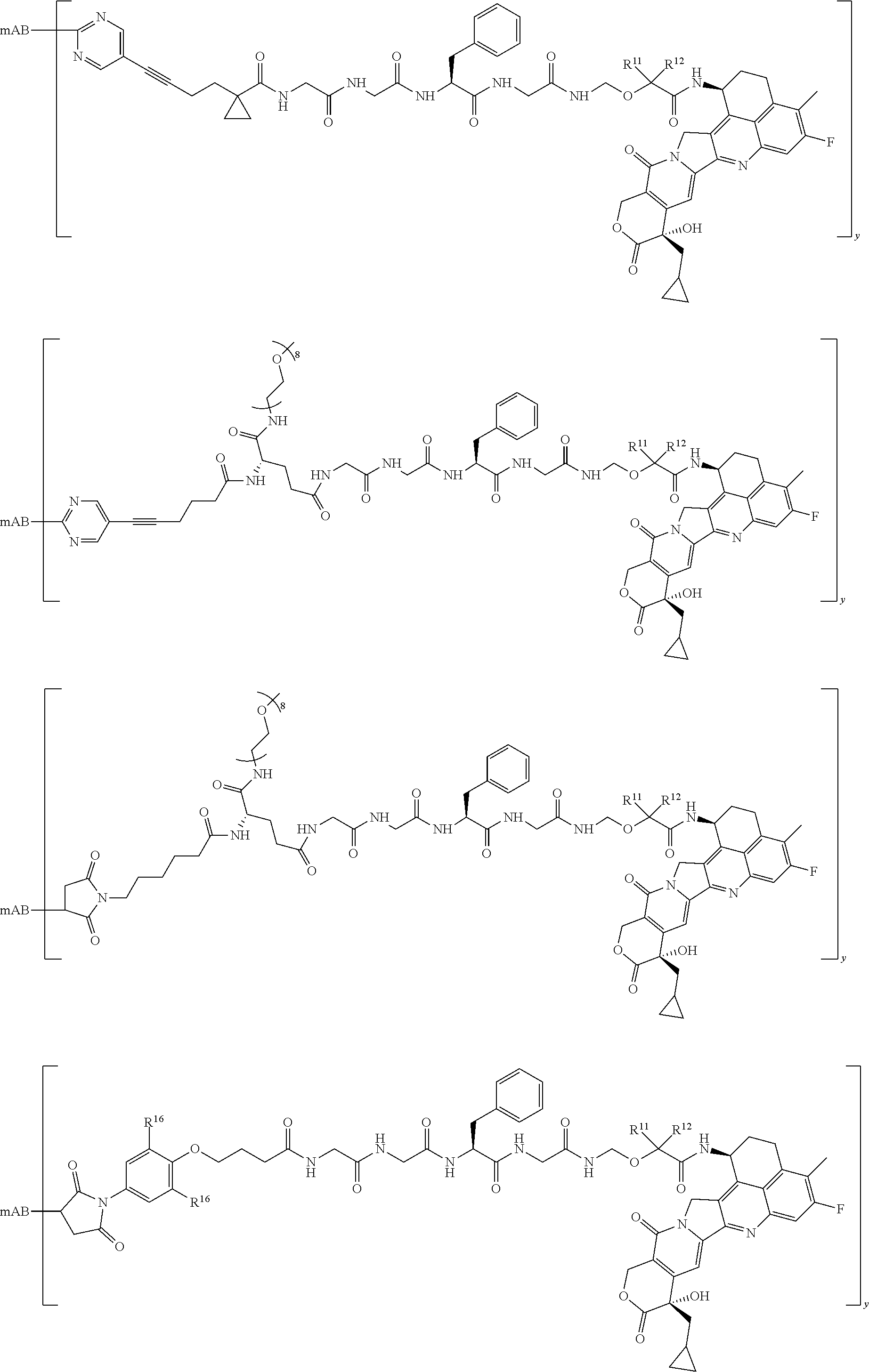

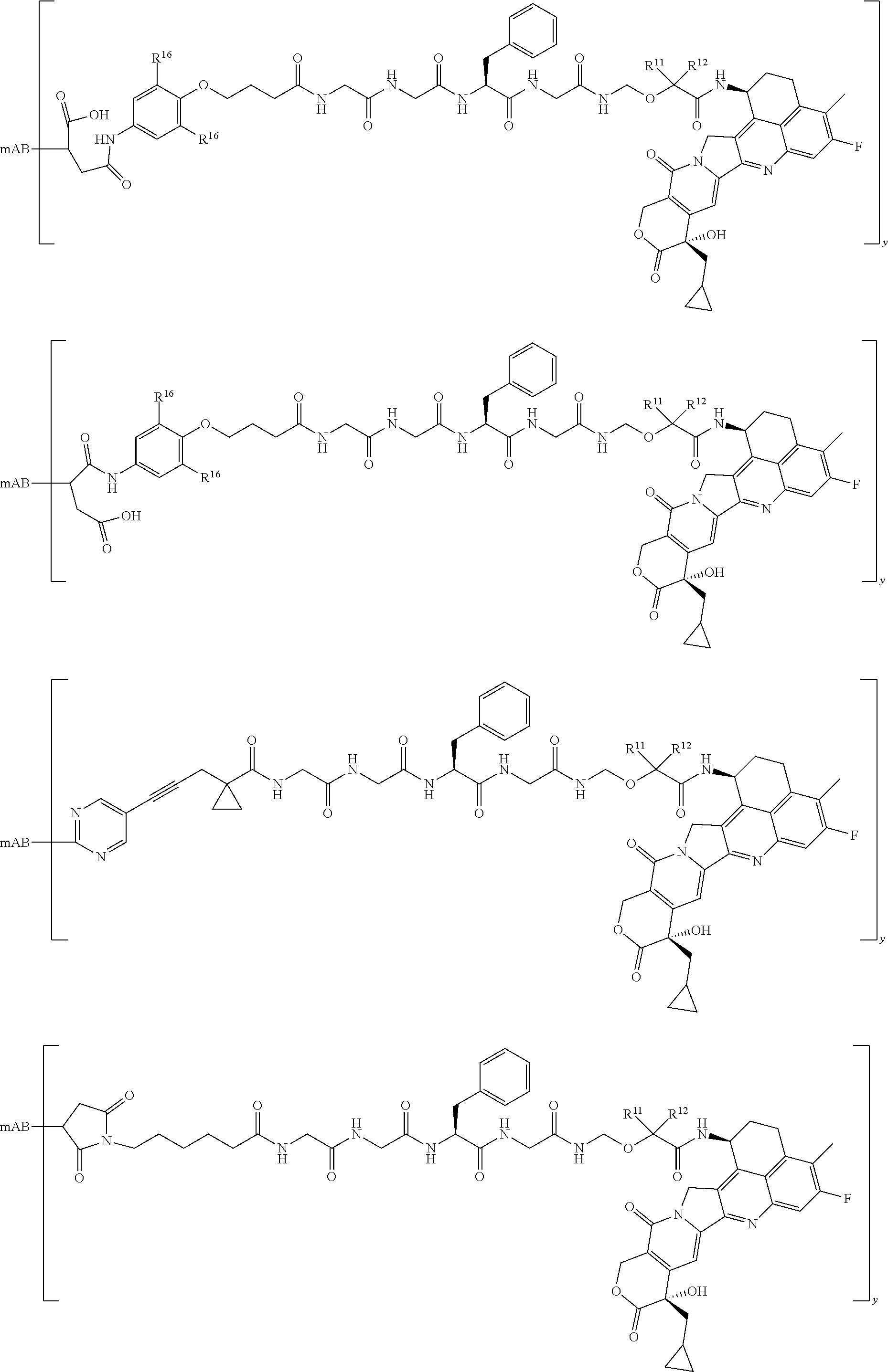

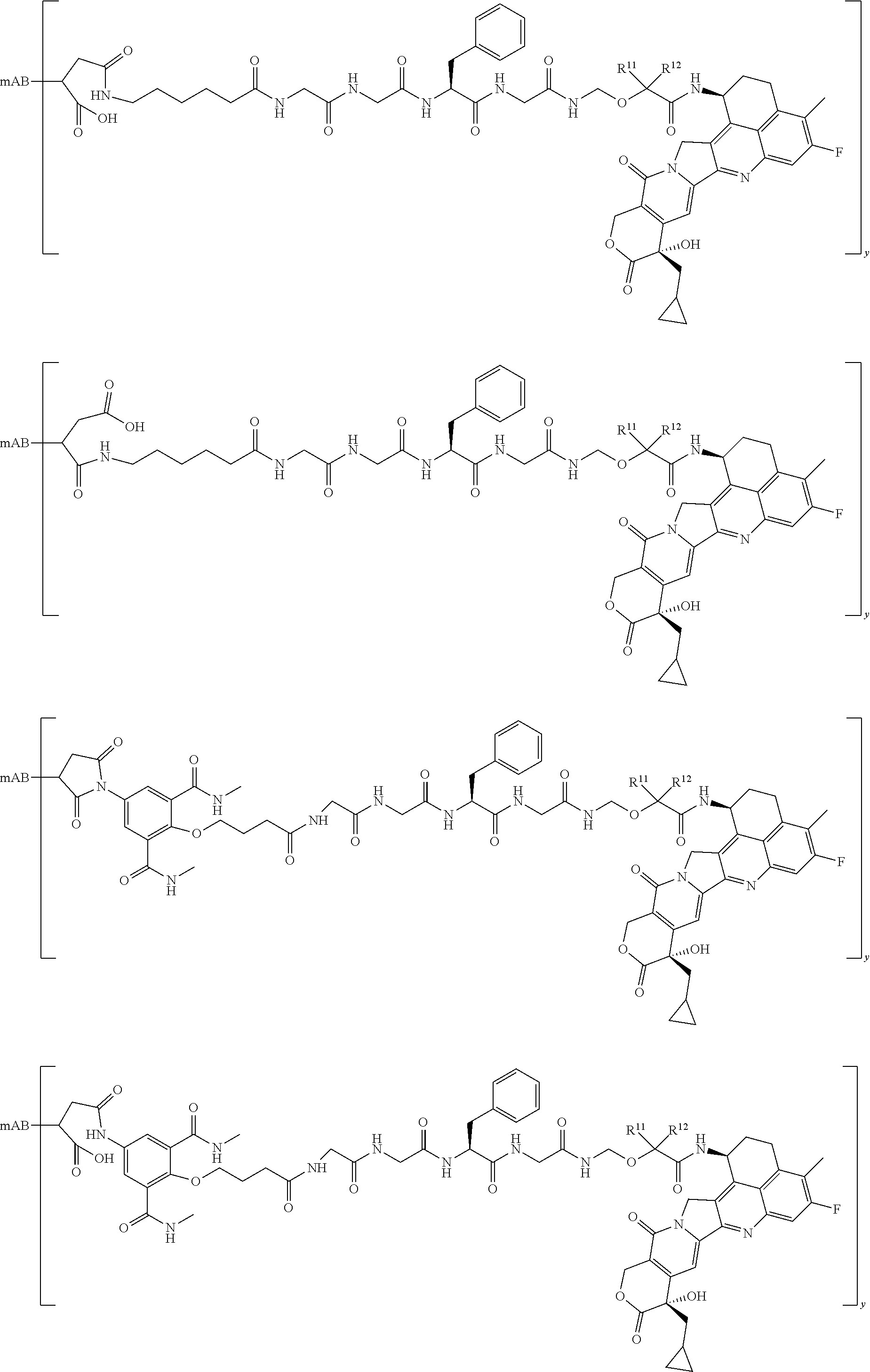

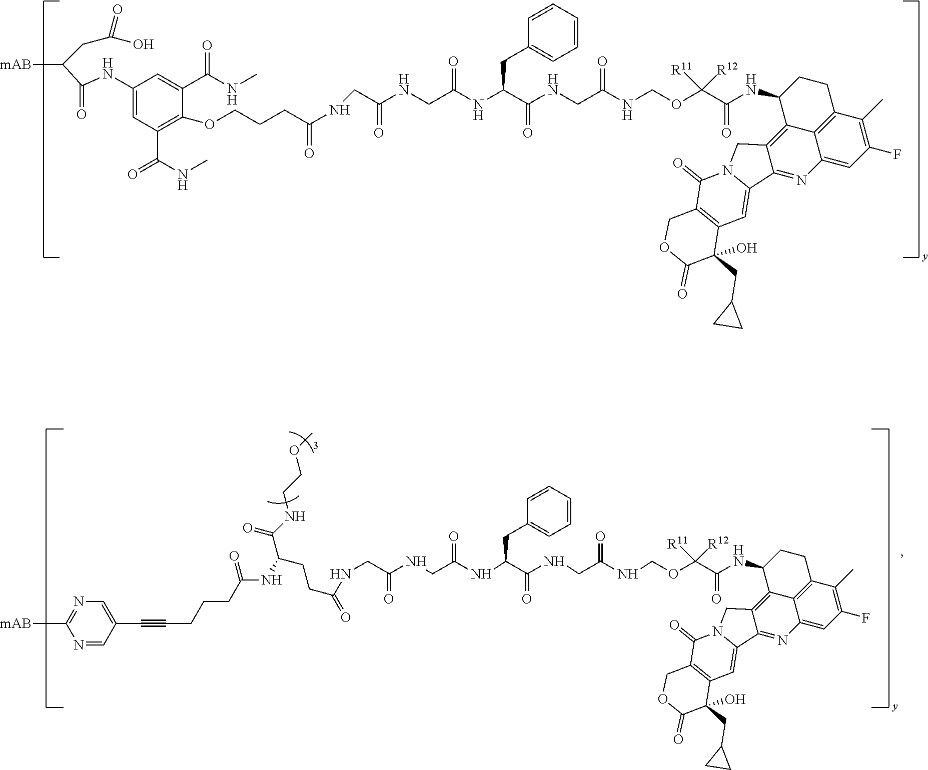

wherein, R¹¹, R¹², R¹⁶, and R¹⁶ have the definitions described above;
for example, R¹¹ and R¹² are the same or different and are independently selected from the group consisting of H, C₁₋₄ alkane C₃₋₆ cycloalkyl, C₃₋₆ cycloalkyl-C₁₋₄ alkyl, or R¹¹ and R¹², together with the carbon atom to which they are attached, form a C₃₋₆ cycloalkyl;
for example, the C₁₋₄ alkyl can be independently selected from the group consisting of methyl, ethyl, propyl, isopropyl, butyl, isobutyl or tert-butyl; the C₃₋₆ cycloalkyl group can be independently selected from the group consisting of cyclopropyl, cyclobutyl, cyclopentyl or cyclohexyl;
for example, both of R¹¹ and R¹² are H, or one of them is not H, or both of them are not H;
mAb represents a monoclonal antibody;
y represents the average number of small molecule drugs attached to each mAb (DAR), which can be selected from an integer or decimal, such as an integer or decimal selected from 1 to 50, an integer or decimal of 1 to 20, or an integer or decimal of 1 to 10.

According to the embodiment of the present disclosure, the conjugate may have a structure as shown in the following formula:

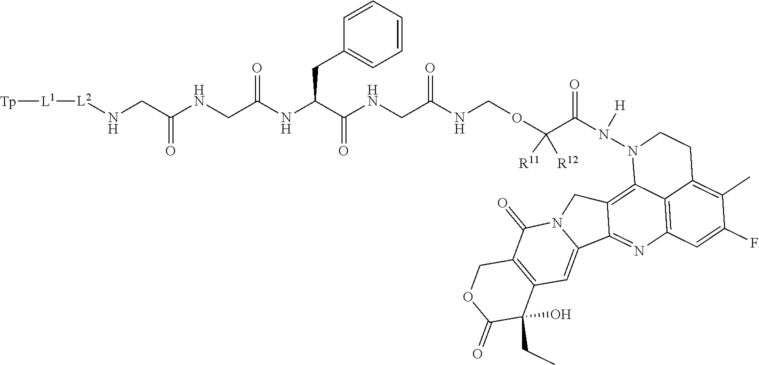

wherein, R¹¹, R¹², L¹, L², and Tp have the definitions as described above.

According to the embodiments of the present disclosure, the conjugate may be one selected from the followings:

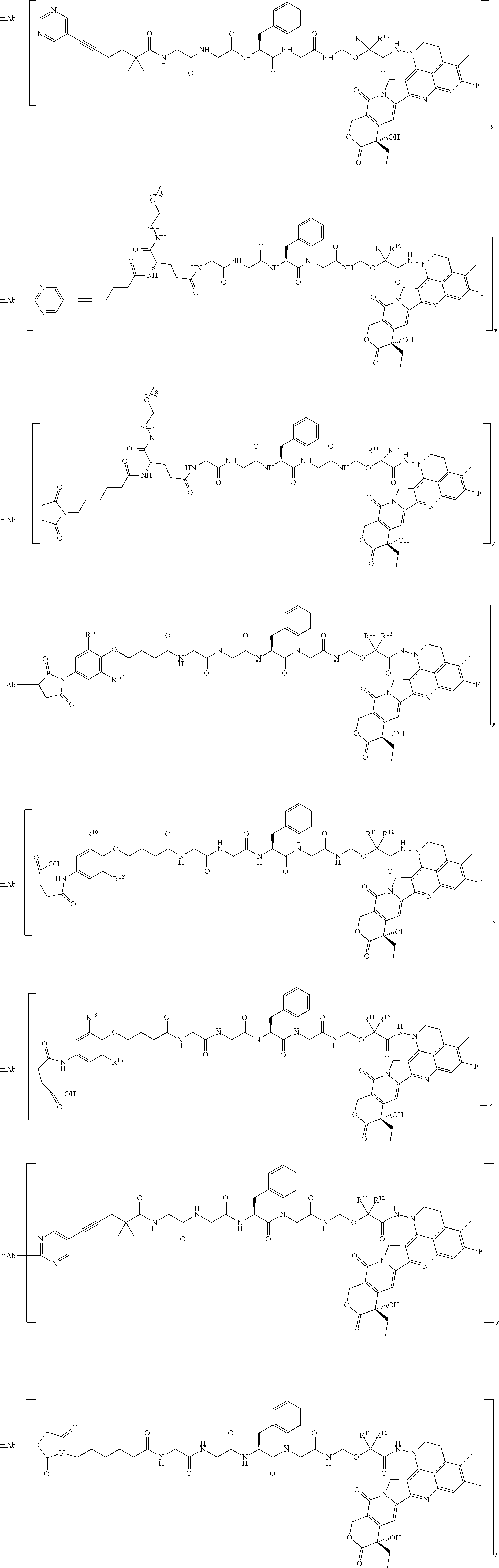

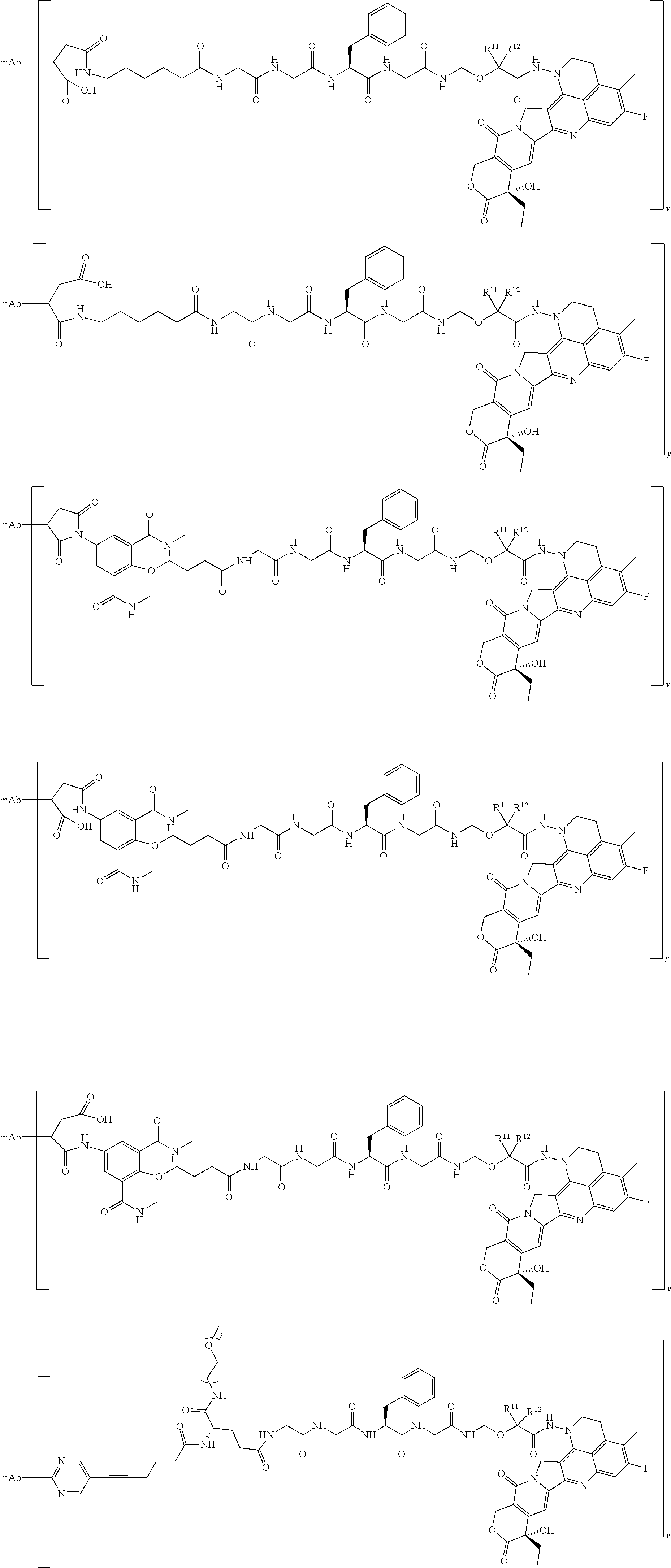

wherein, R¹¹, R¹², R¹⁶, and R^{16′} independently have the definitions as described above;
mAb represents a monoclonal antibody;
y represents the average number of small molecule drugs attached to each monoclonal antibody (DAR), and can be selected from an integer or decimal, such as an integer or decimal of 1 to 50, an integer or decimal of 1 to 20, or an integer or decimal of 1 to 10.

According to the embodiment of the present disclosure, the conjugate may have a structure as shown in the following formula:

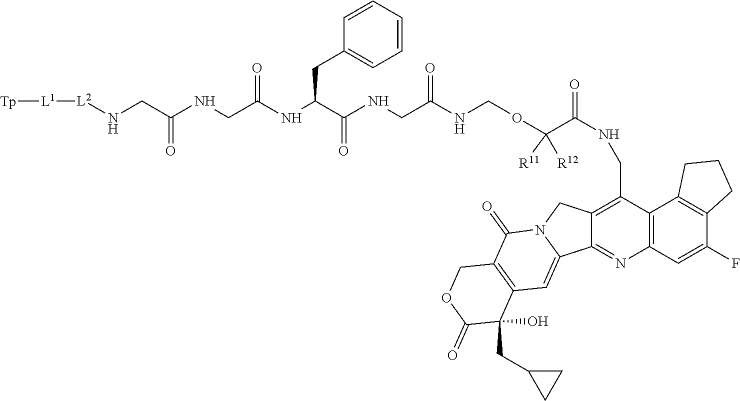

wherein, R¹¹, R¹², L¹, L², and Tp have the definitions as described above.

According to the embodiments of the present disclosure, the conjugate can be one selected from the followings:

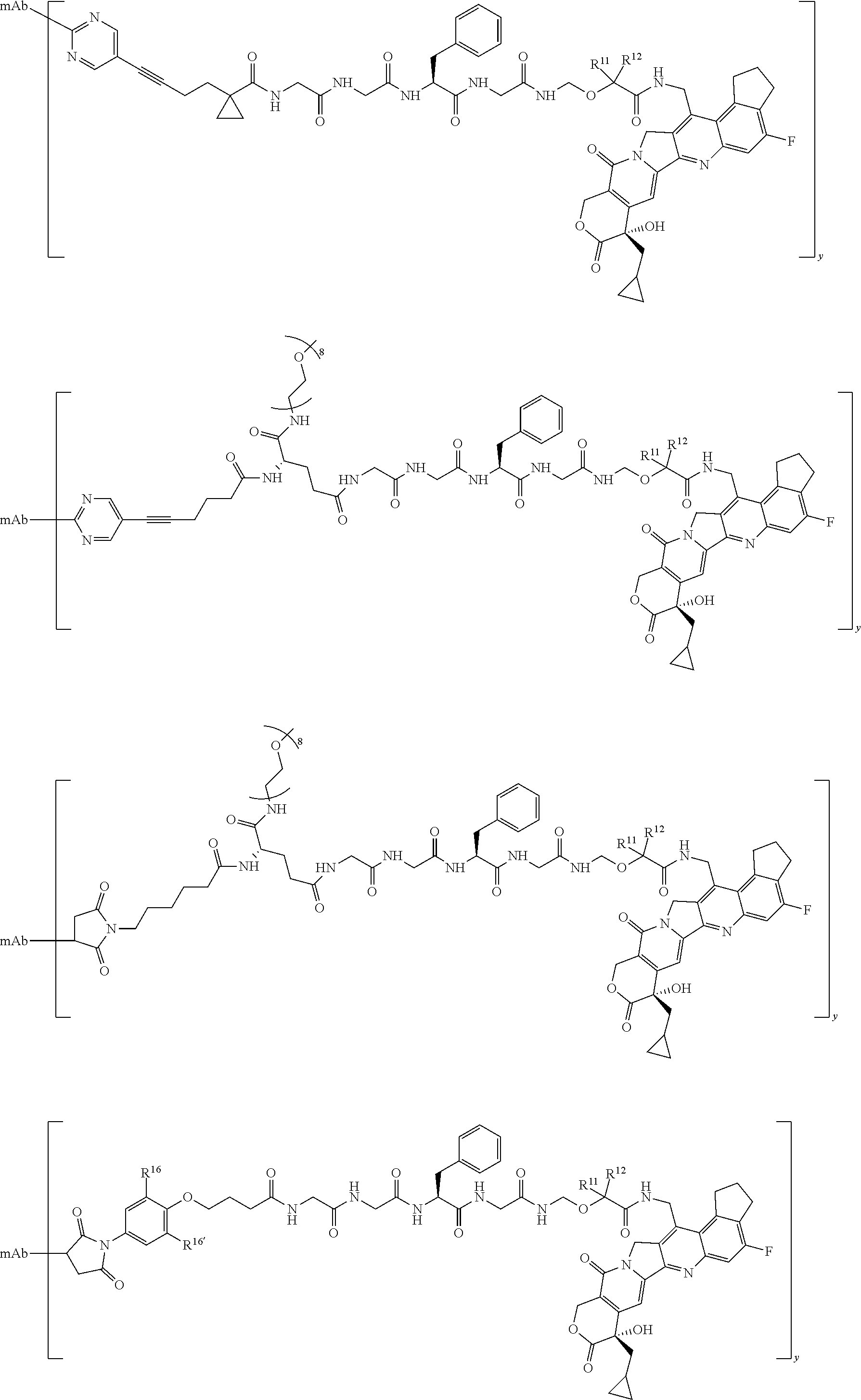

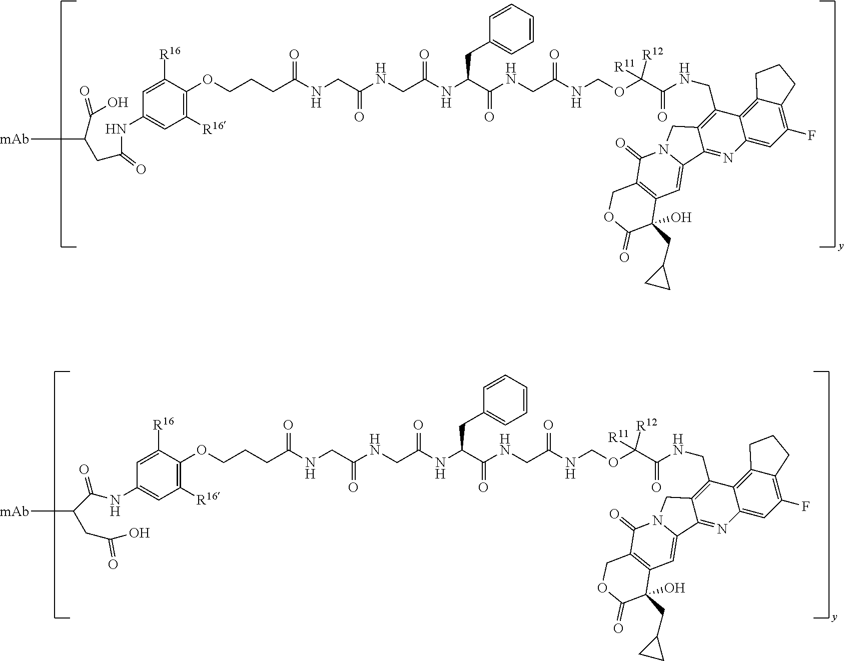

wherein, R¹¹, R¹², R¹⁶, and R^{16′} have the definitions as described above;
mAb represents a monoclonal antibody;
y represents the average number of small molecule drugs attached to each monoclonal antibody (DAR), which can be selected from an integer or decimal, such as an integer or decimal selected from 1 to 50, an integer or decimal selected from 1 to 20, or an integer or decimal selected from 1 to 10.

According to the embodiment of the present disclosure, examples of the conjugate intermediates, namely linker-drugs, may be selected from one of the followings:

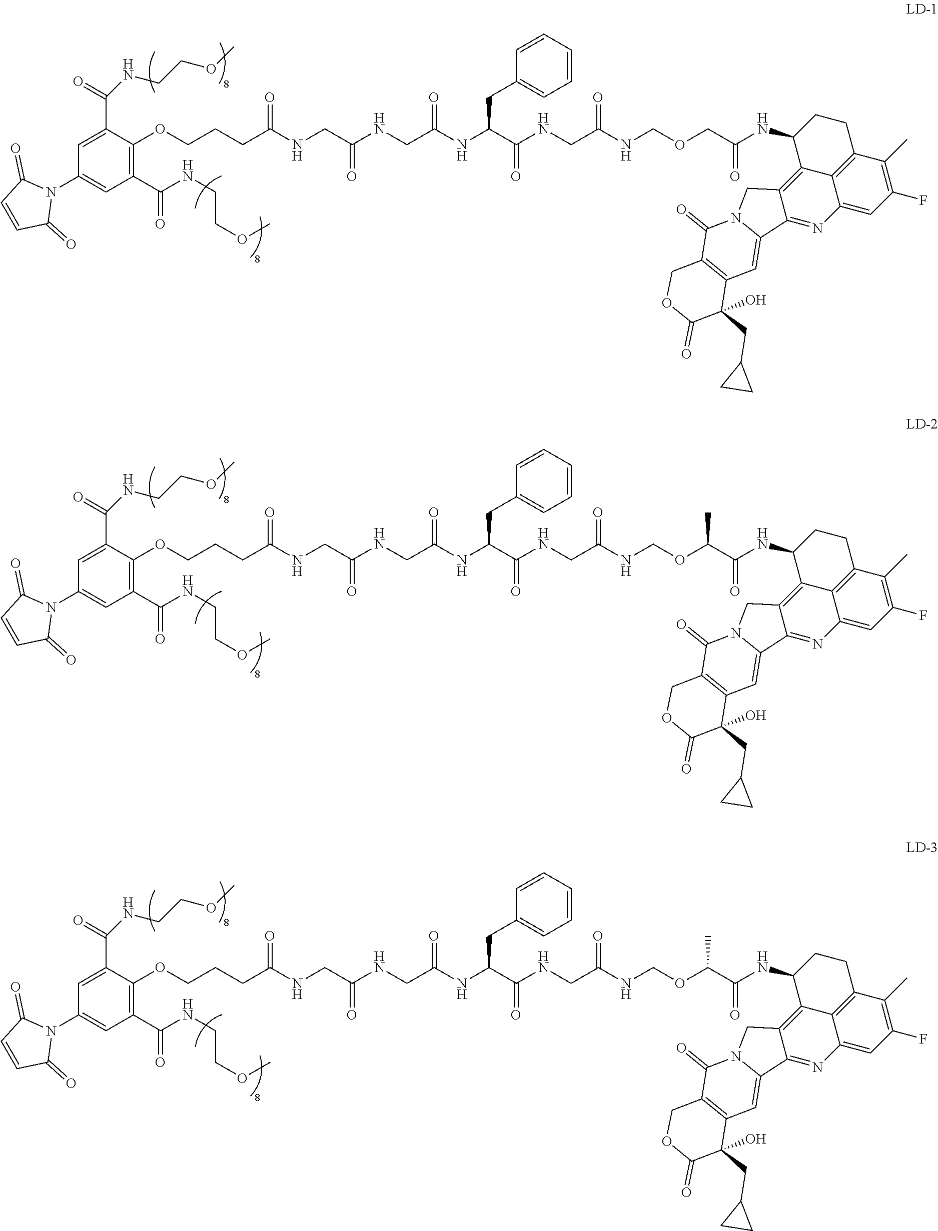

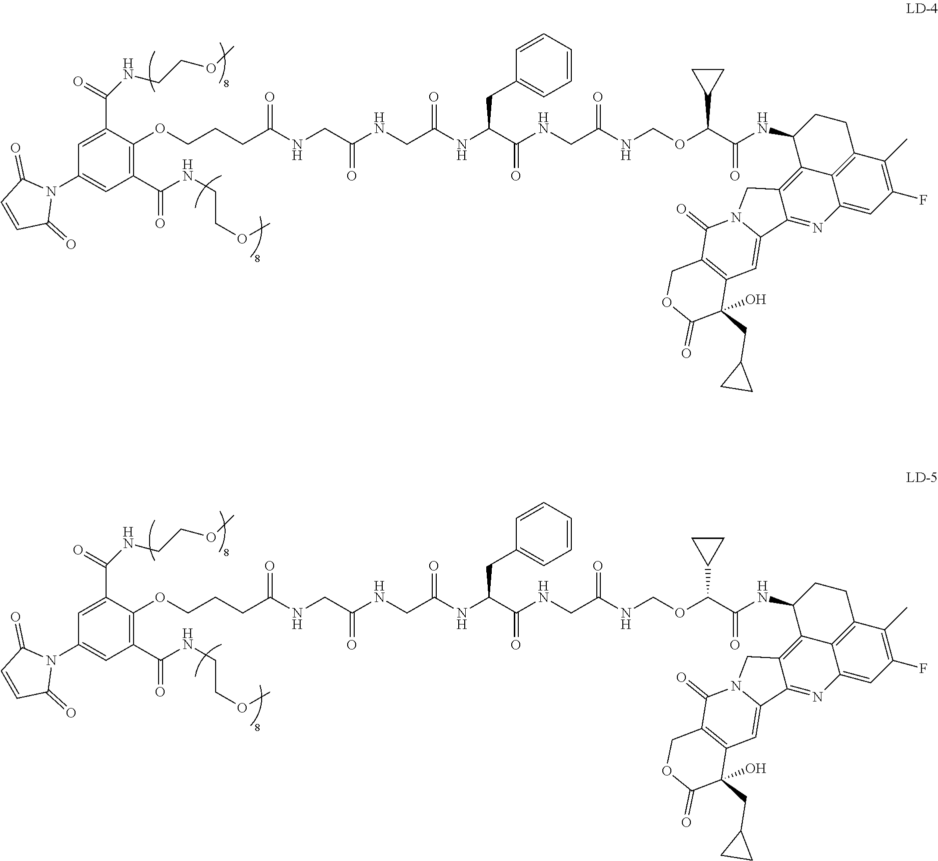

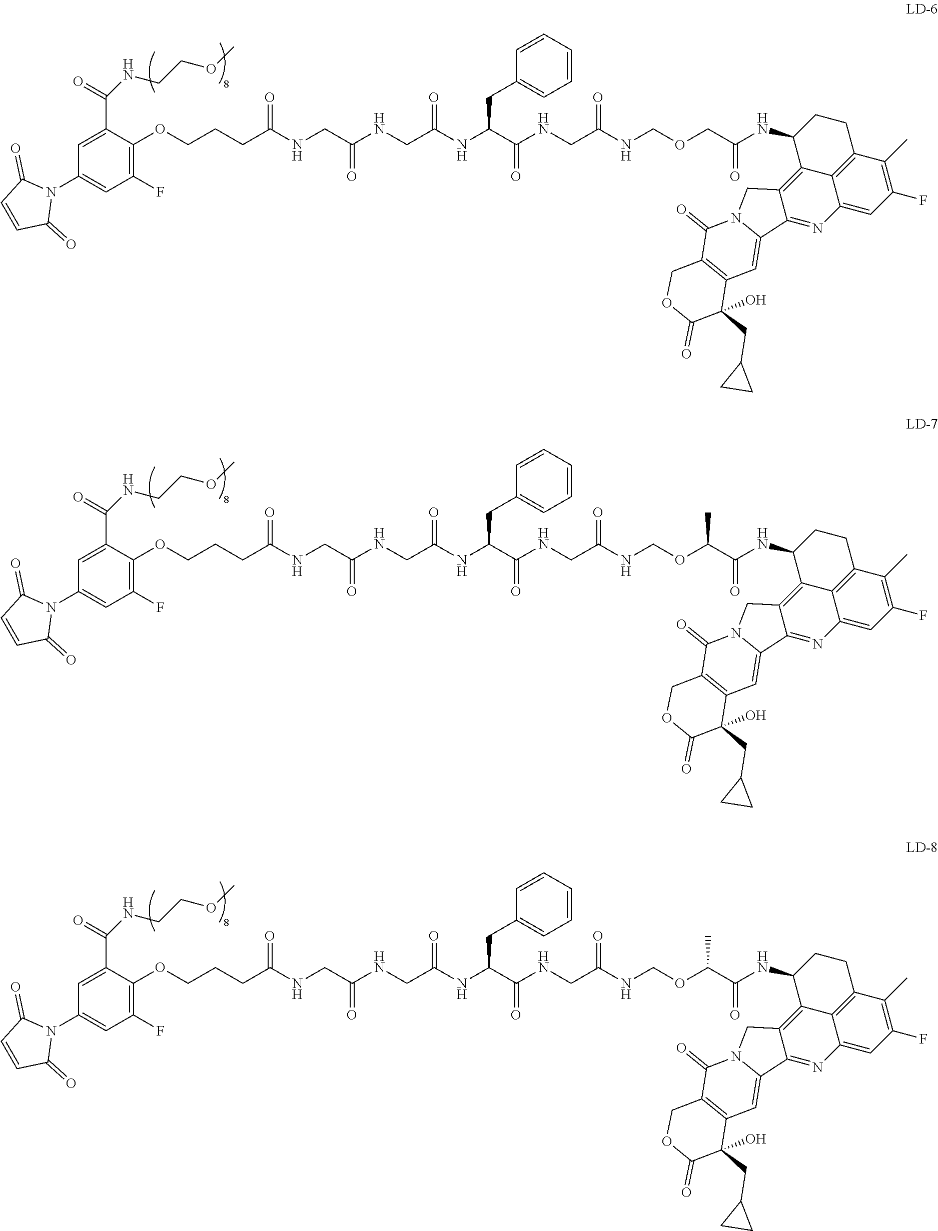

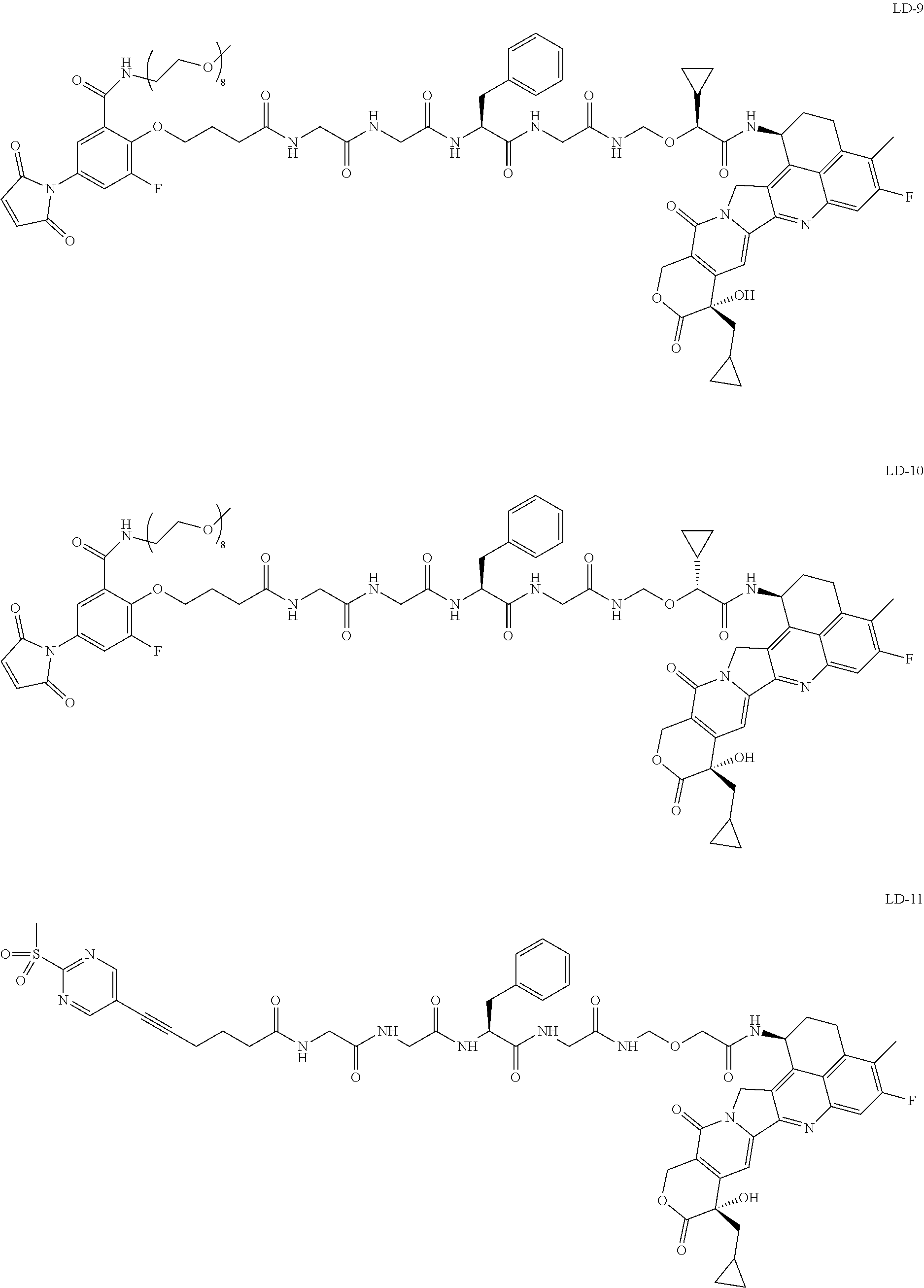

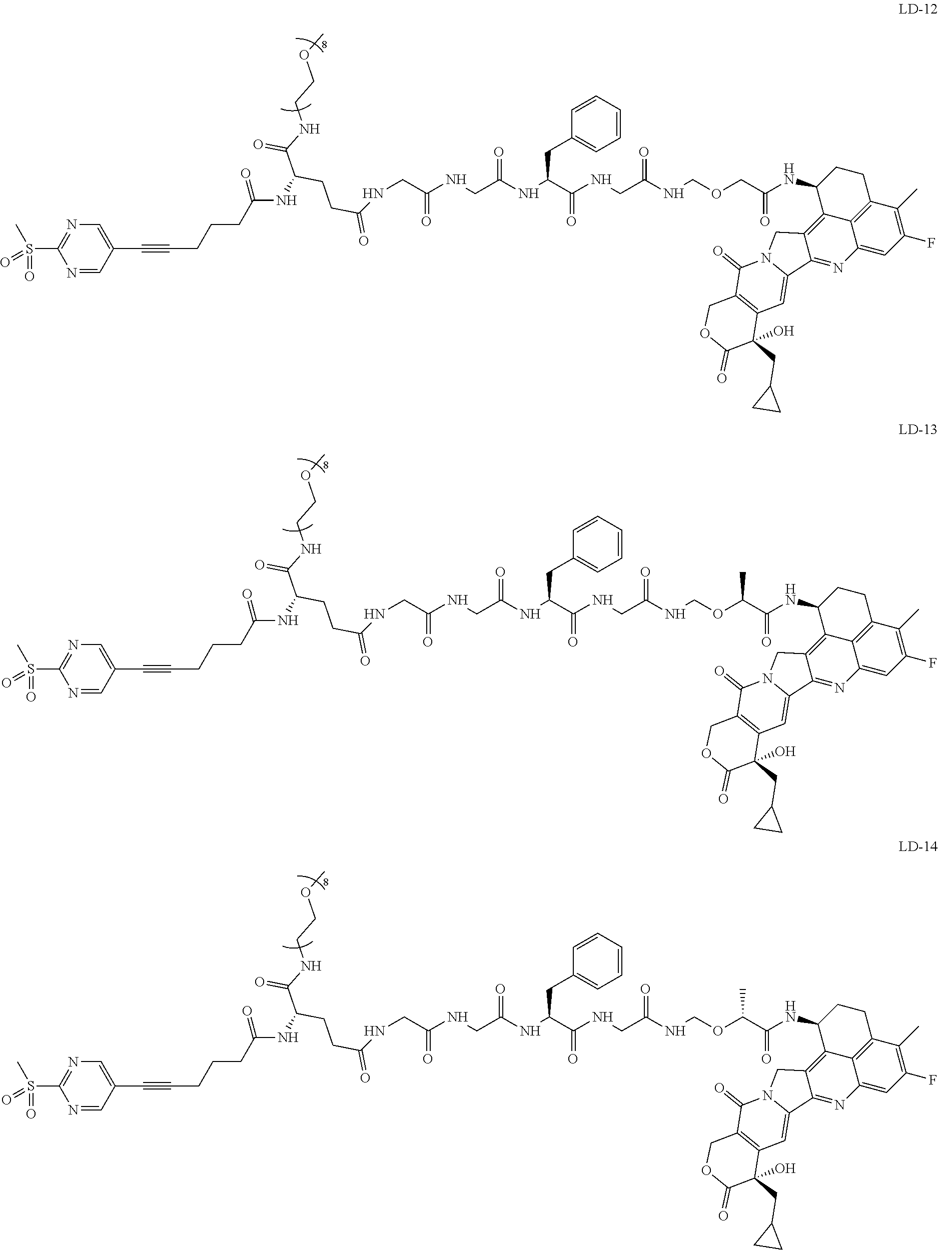

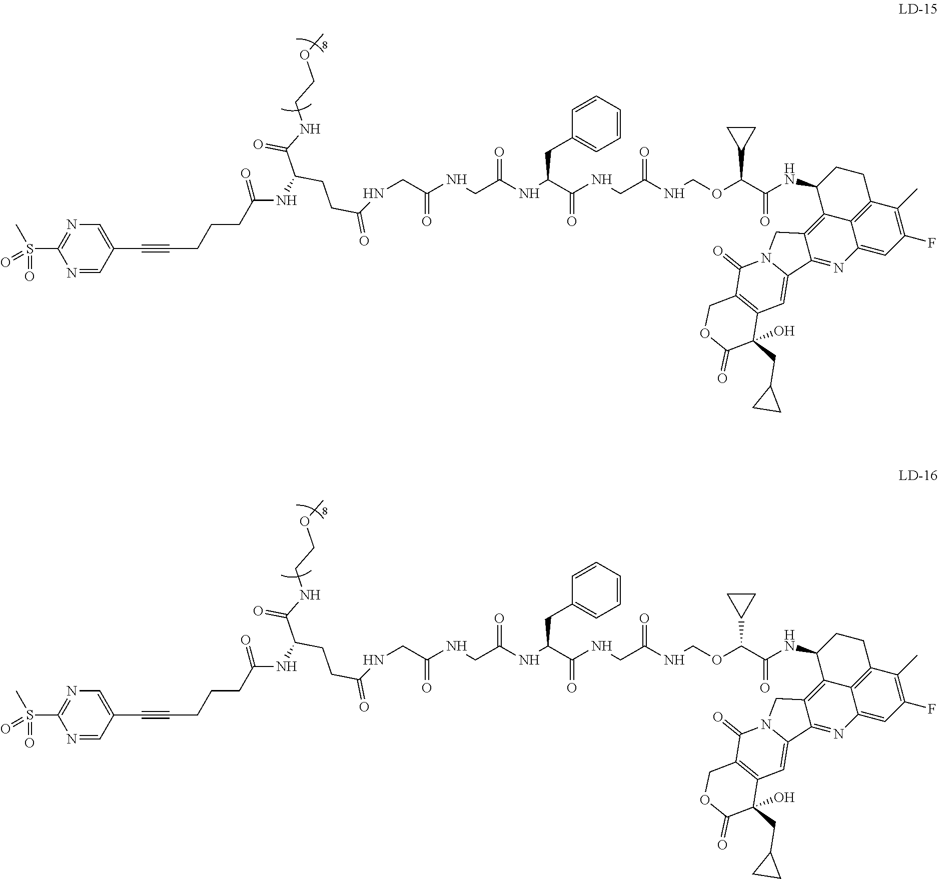

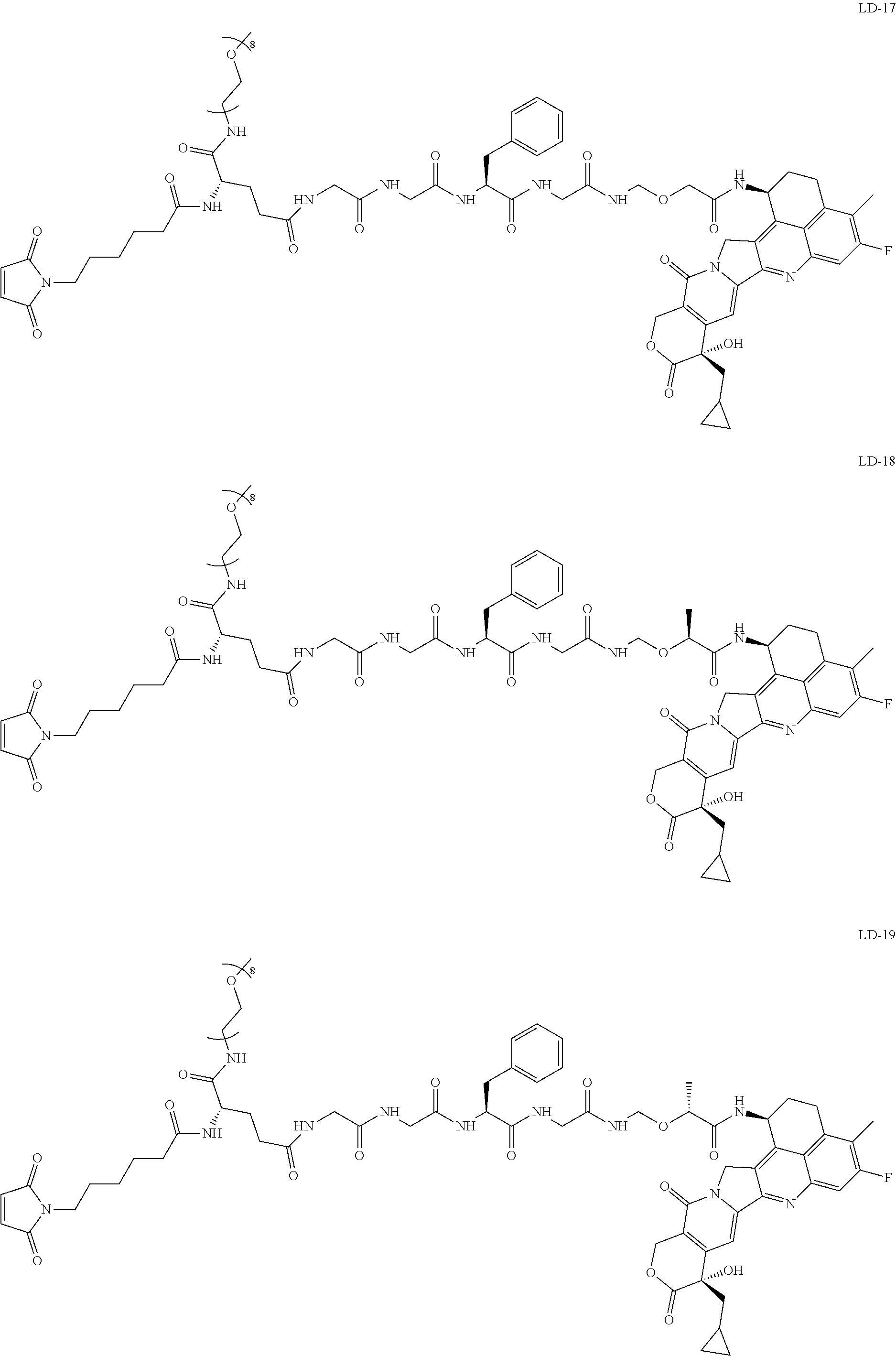

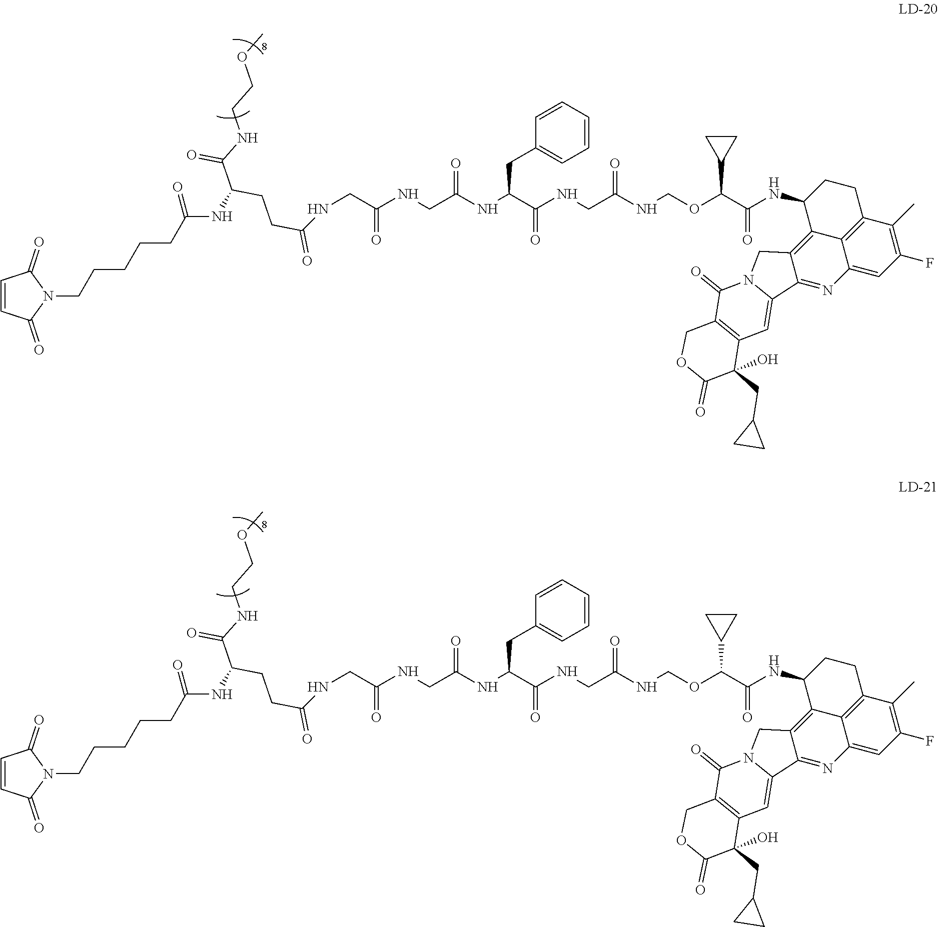

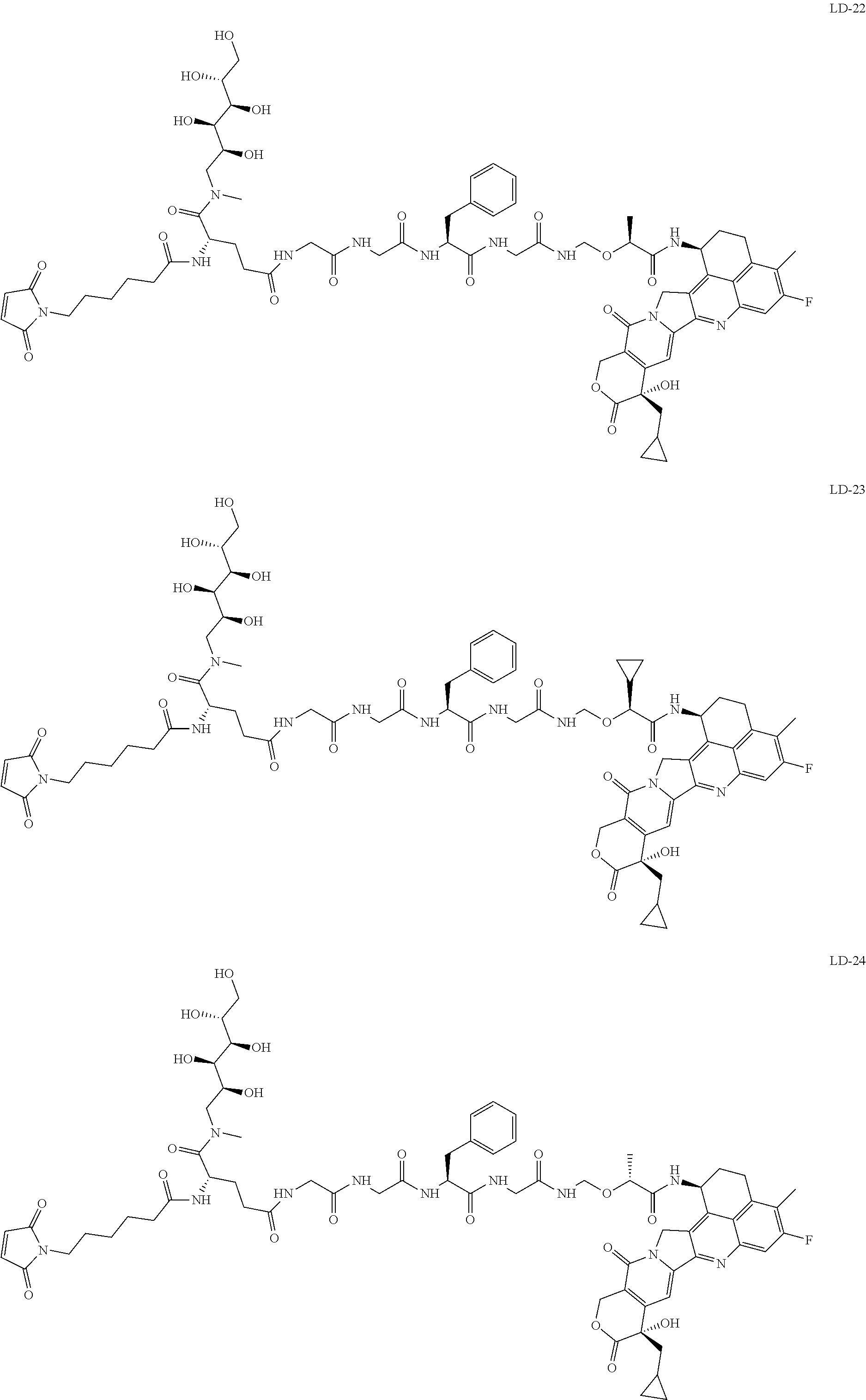

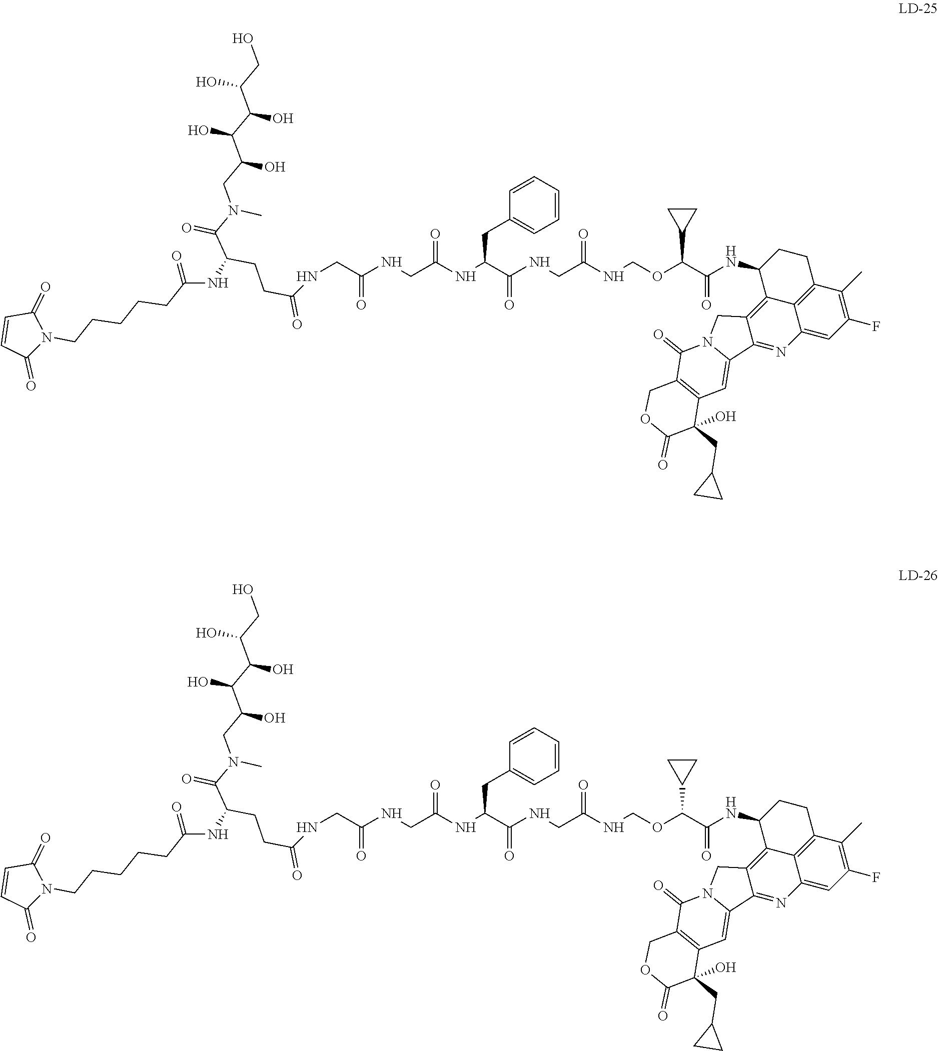 [SMILES: Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)Cn1c-3cc2c(c1=O)COC(=O)[C@]2(O)CC1CC1 ; Cc1c(F)cc2nc3c(c4c2c1CC[C@@H]4NC(=O)[C@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)Cn1c-3cc2c(c1=O)COC(=O)[C@]2(O)CC1CC1]

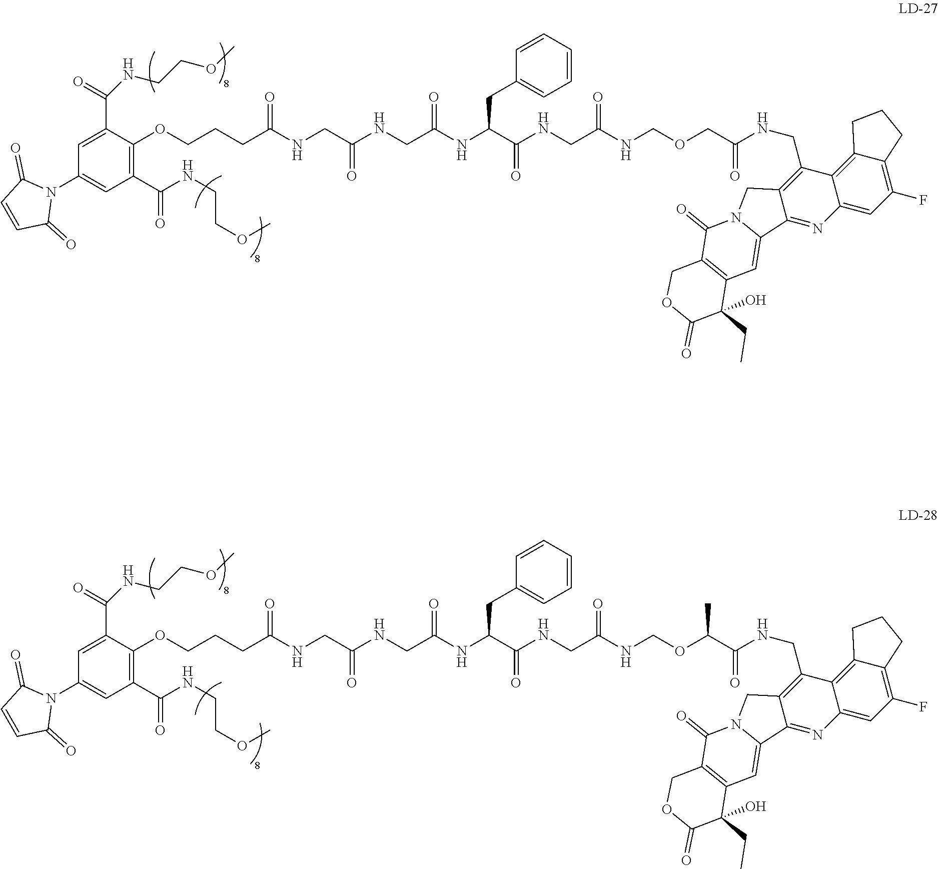

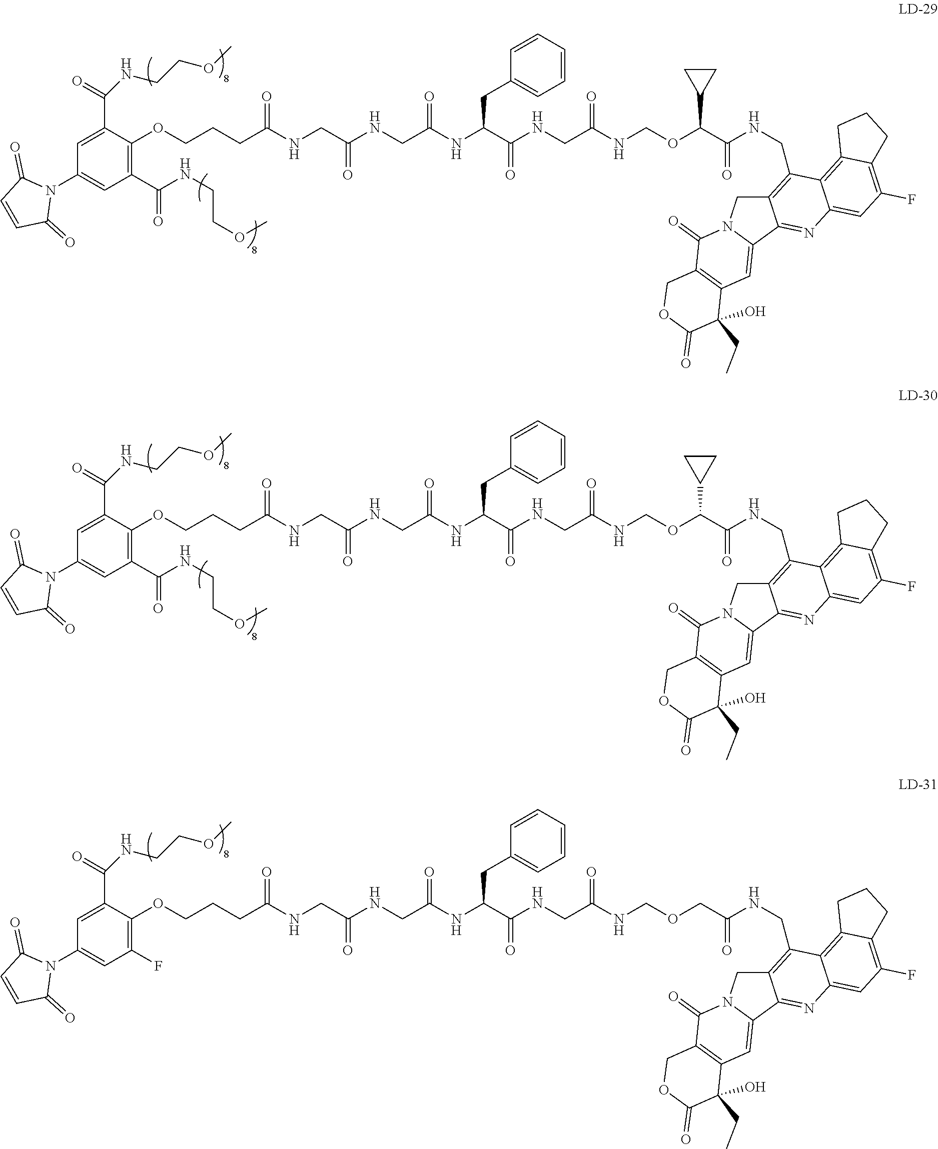

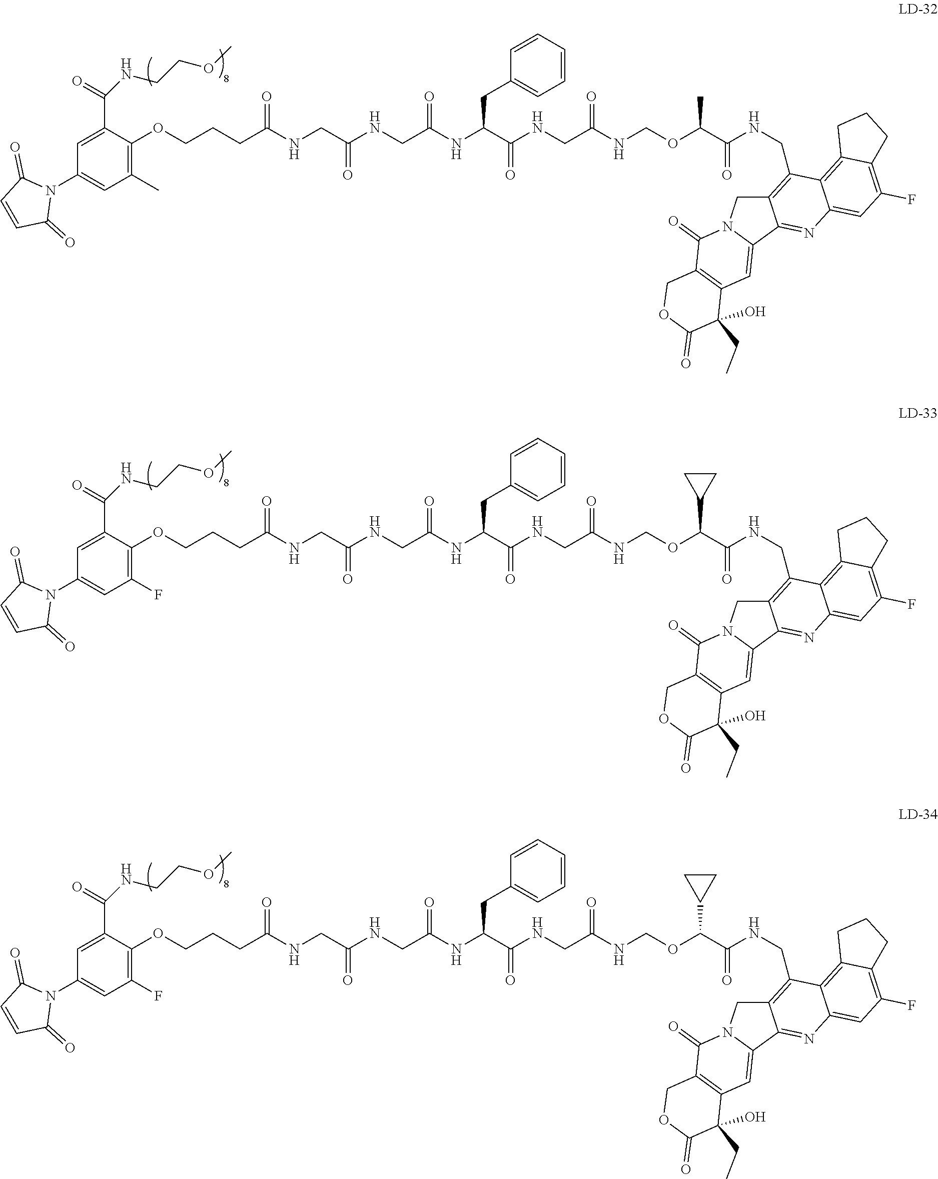

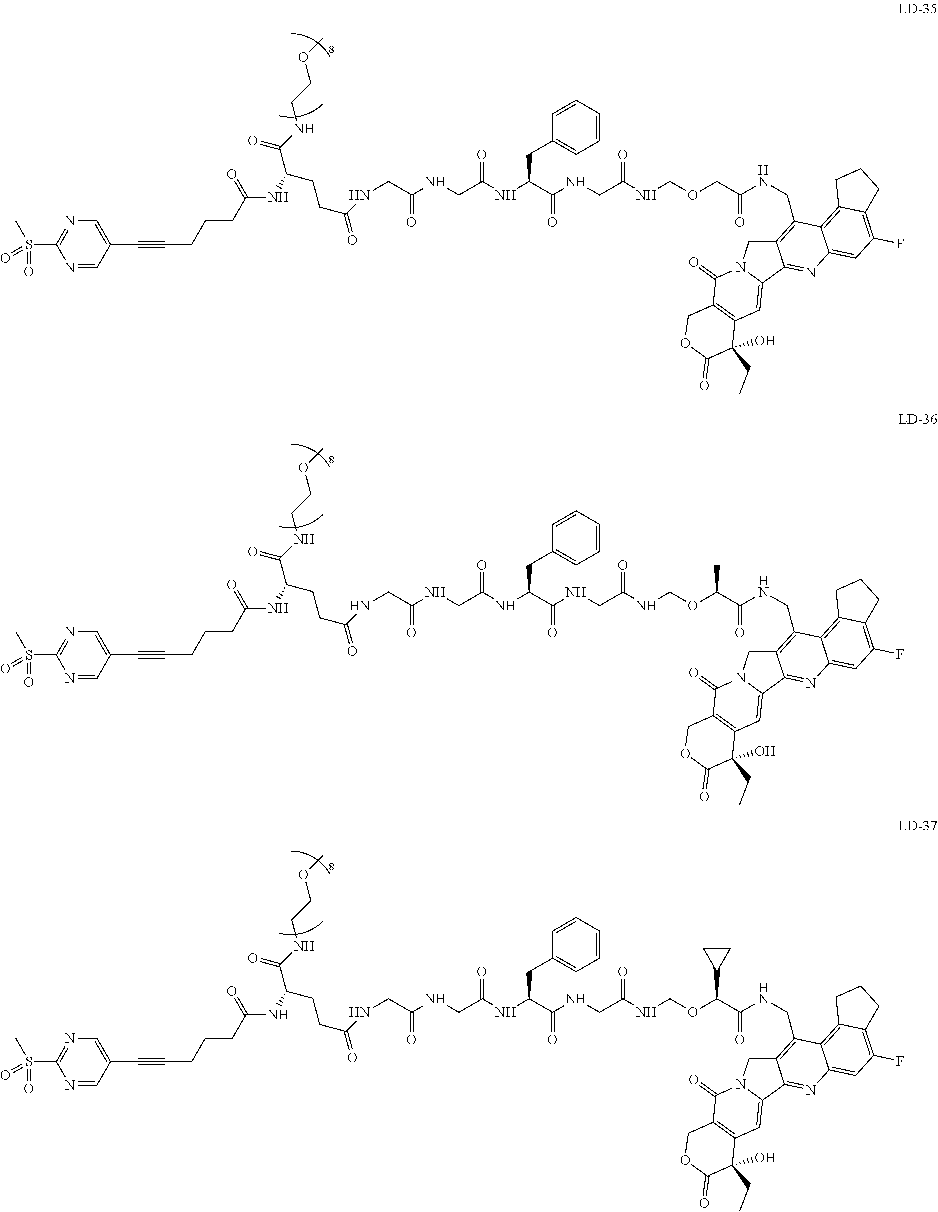

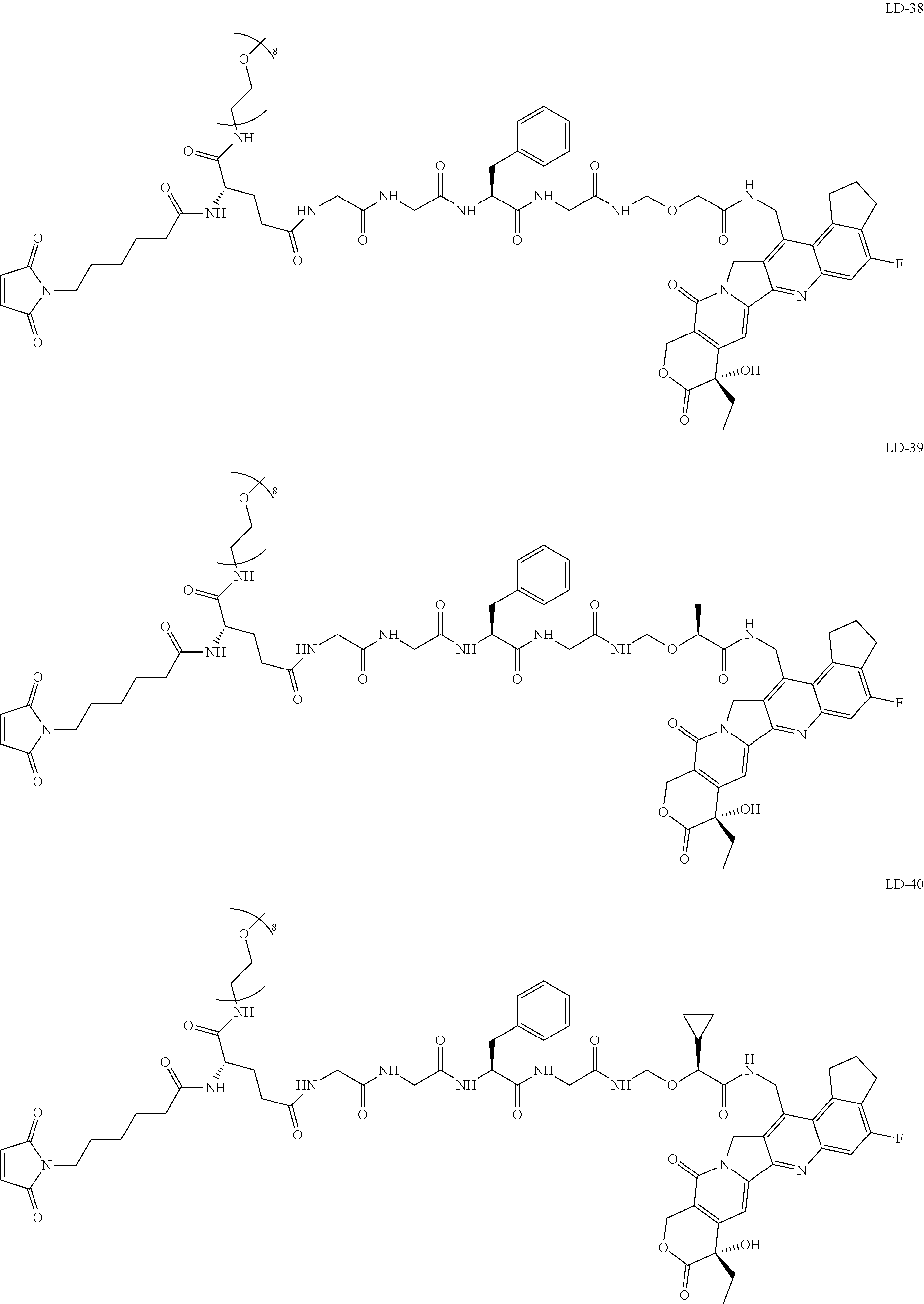

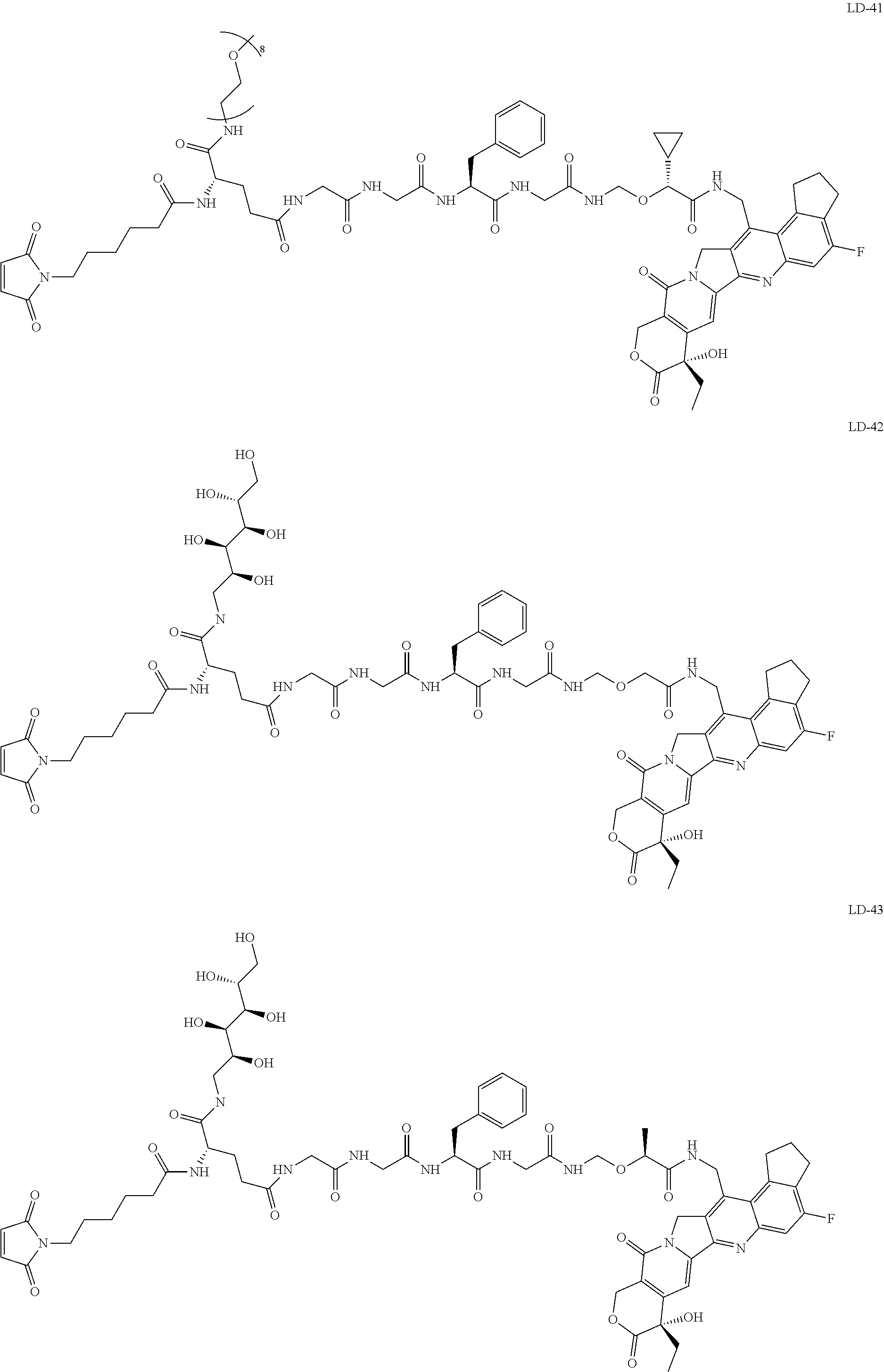

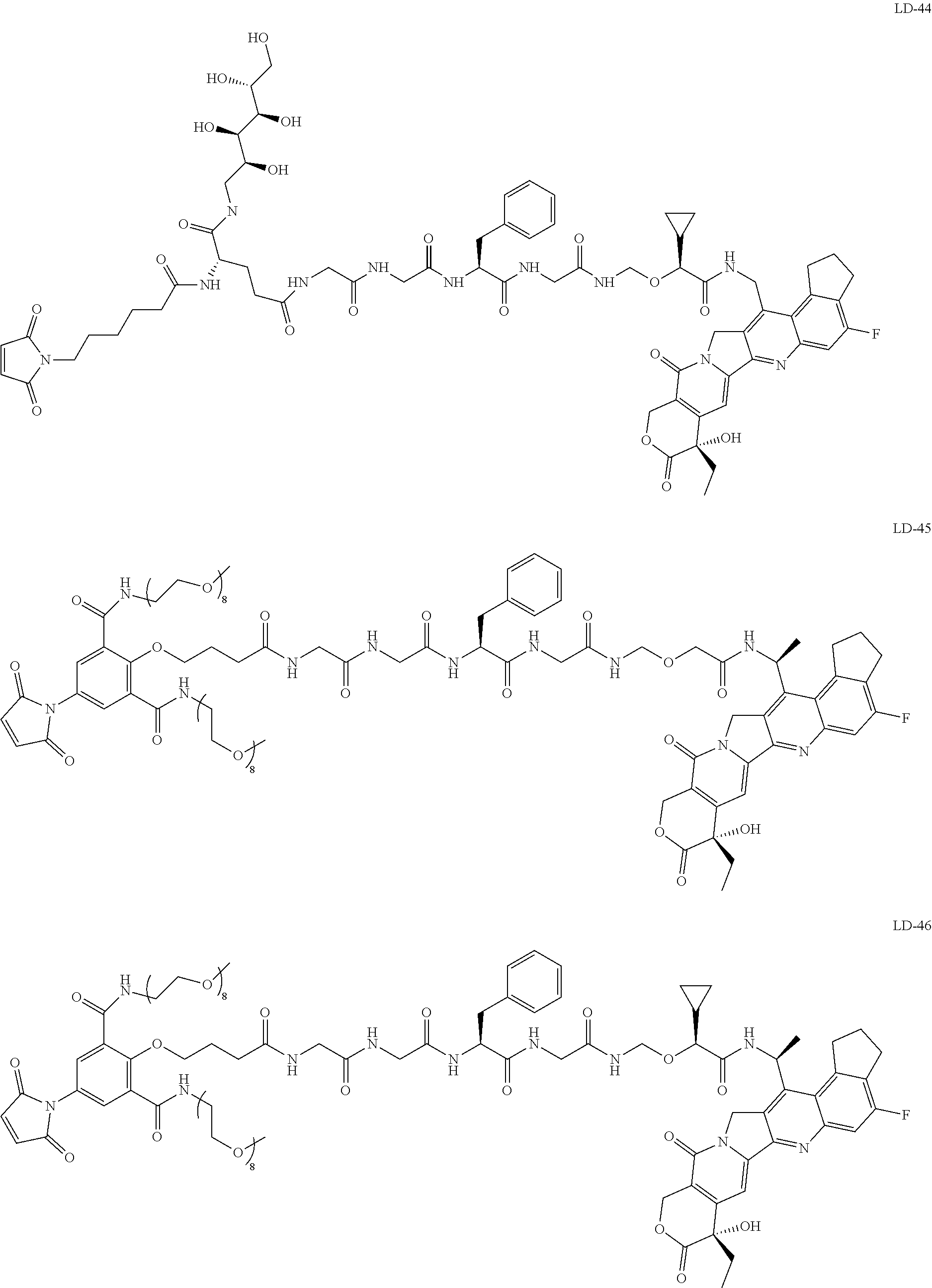

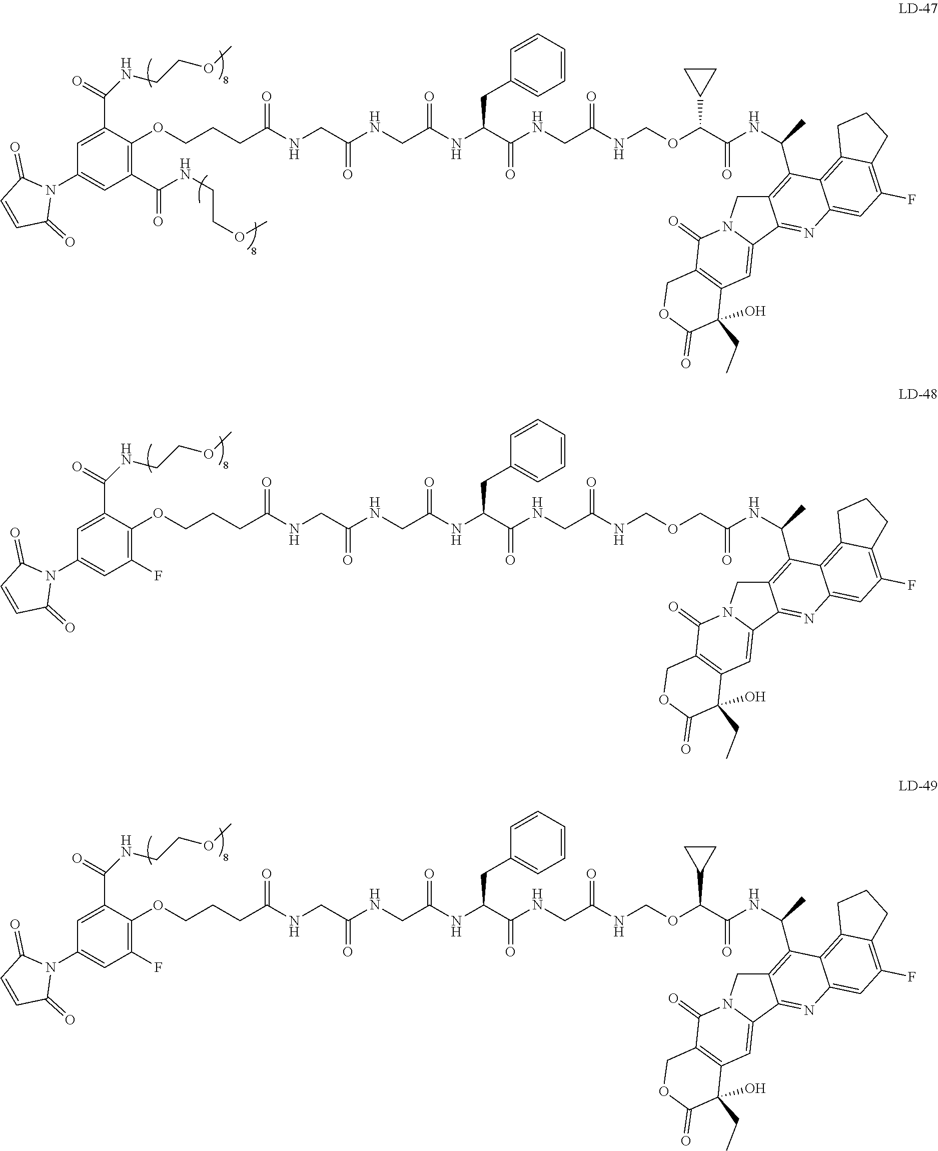

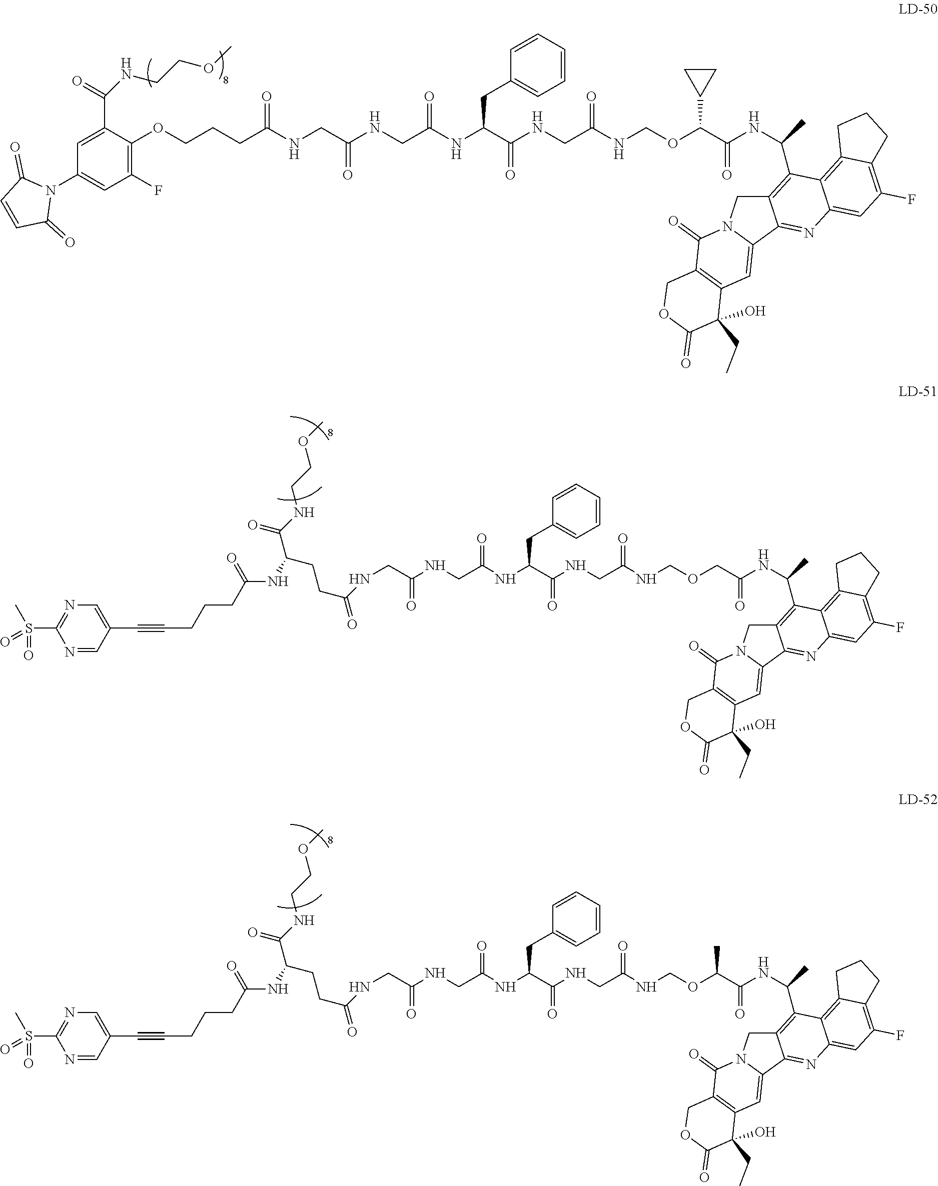

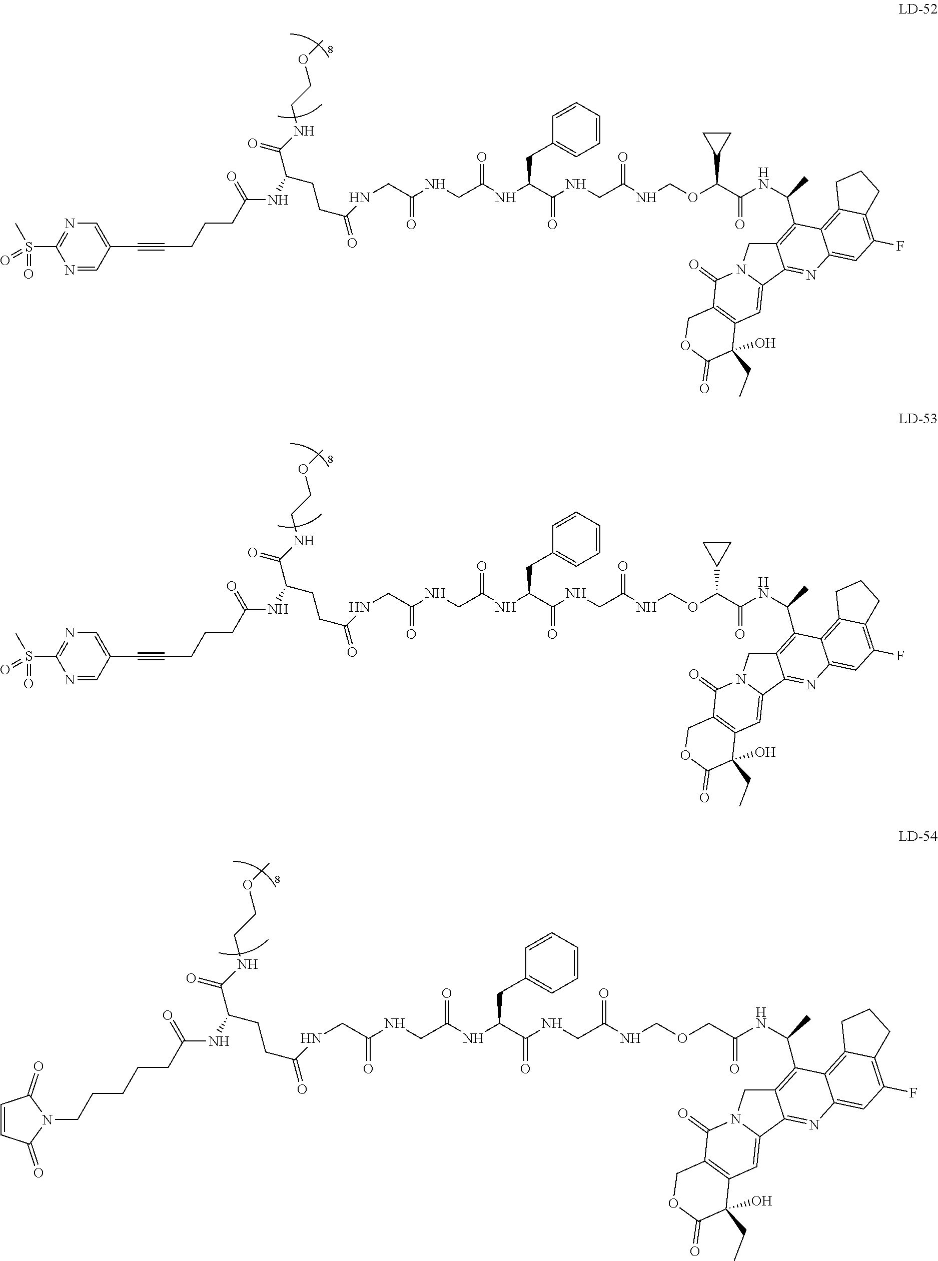

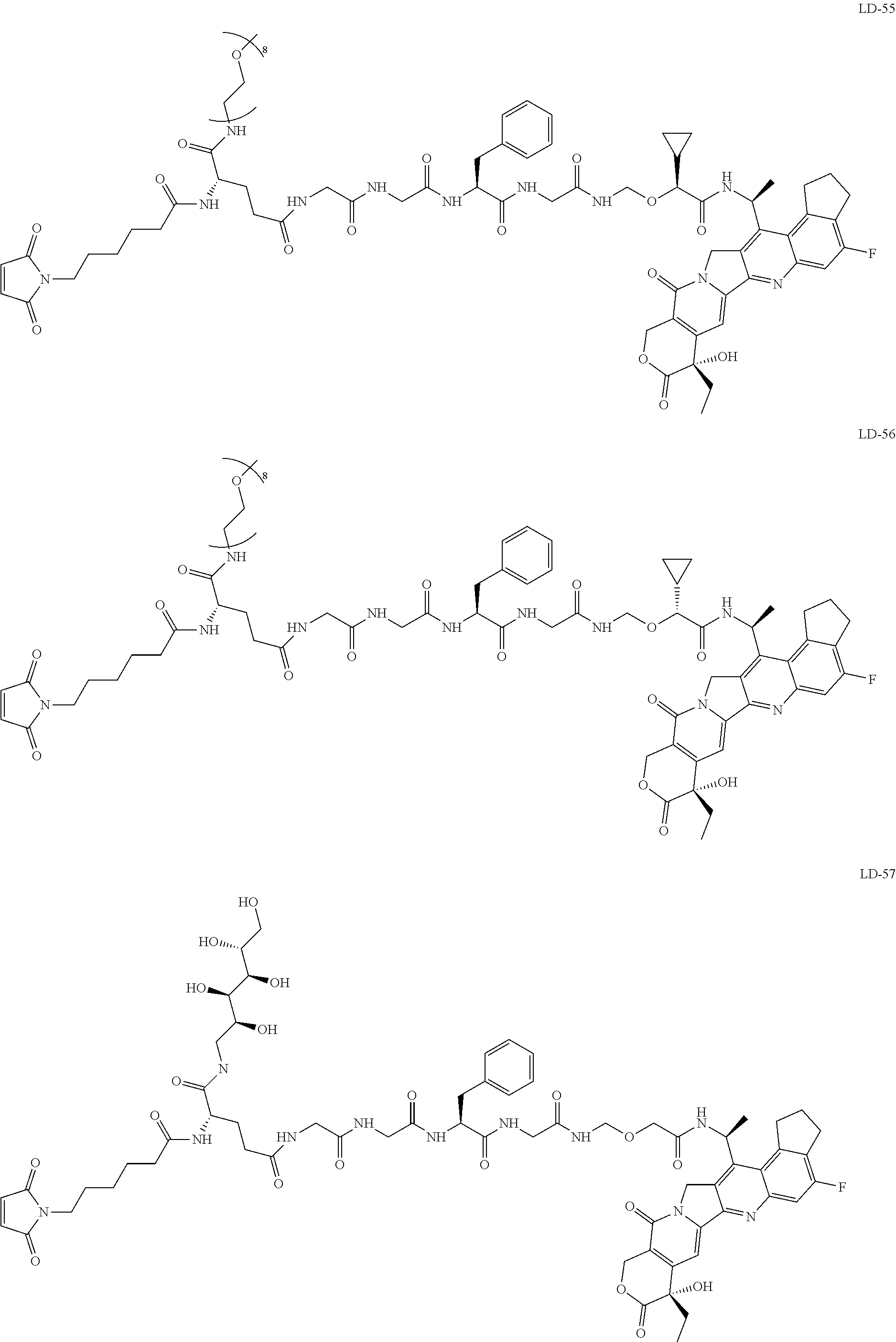

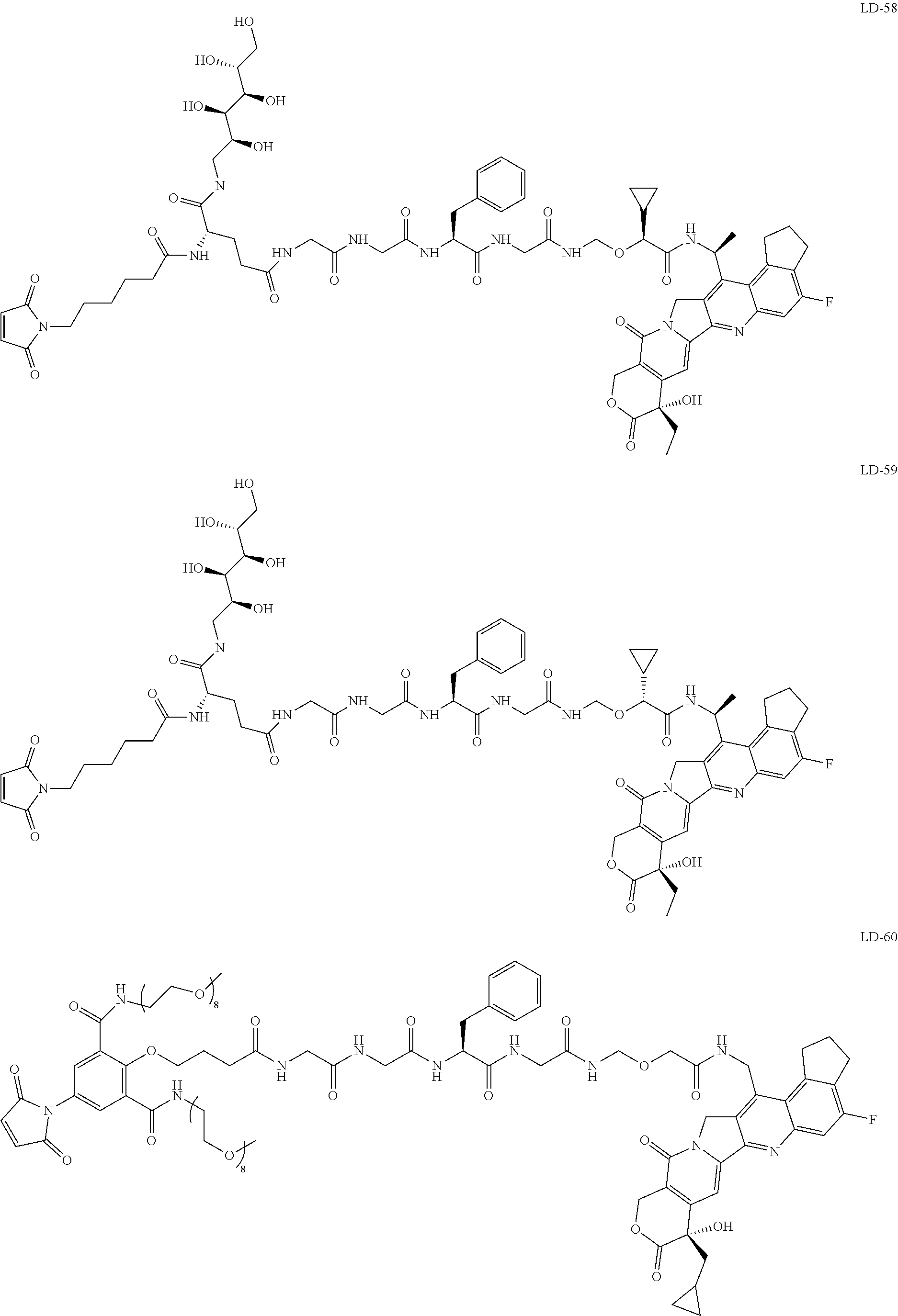

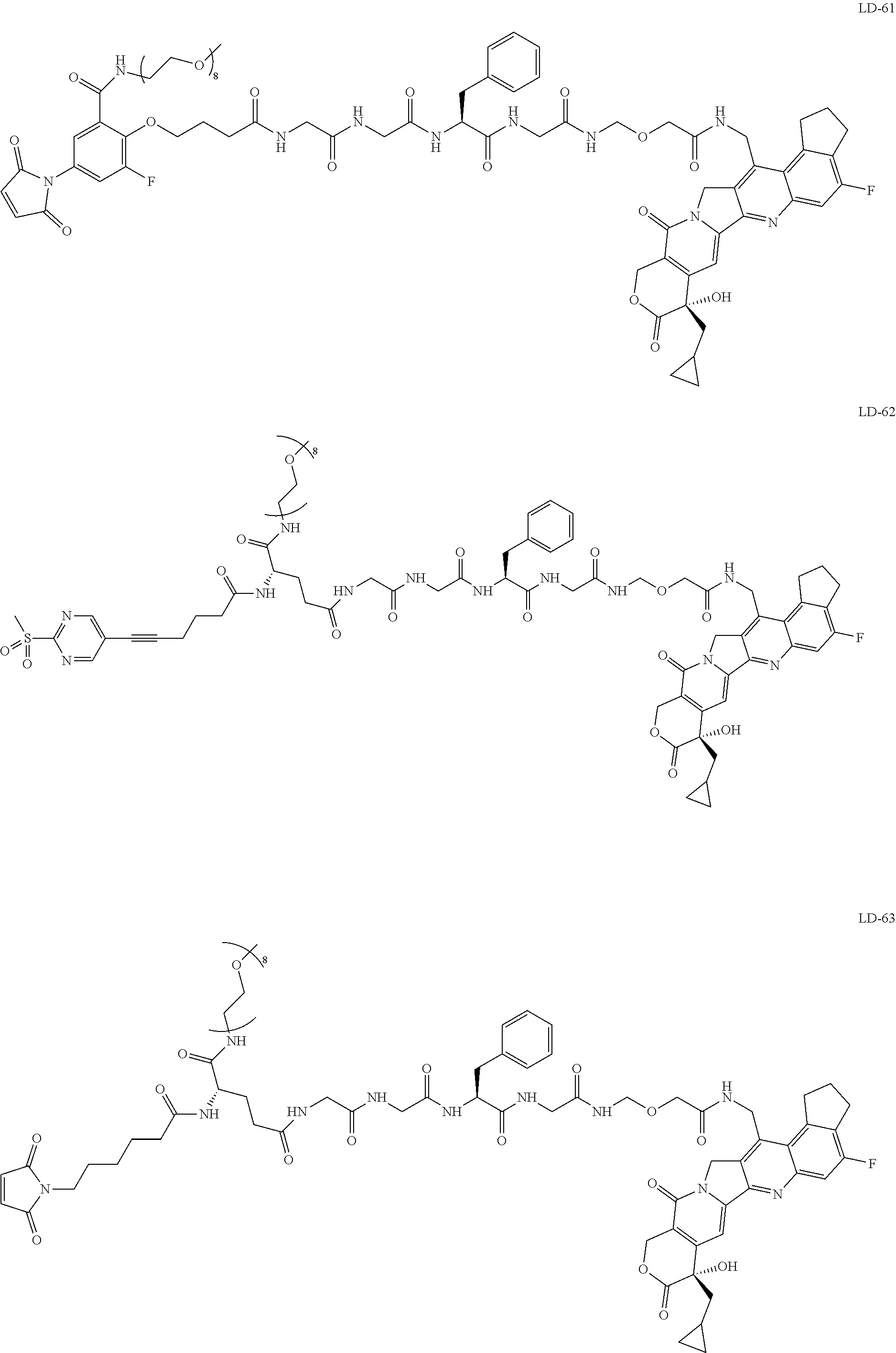

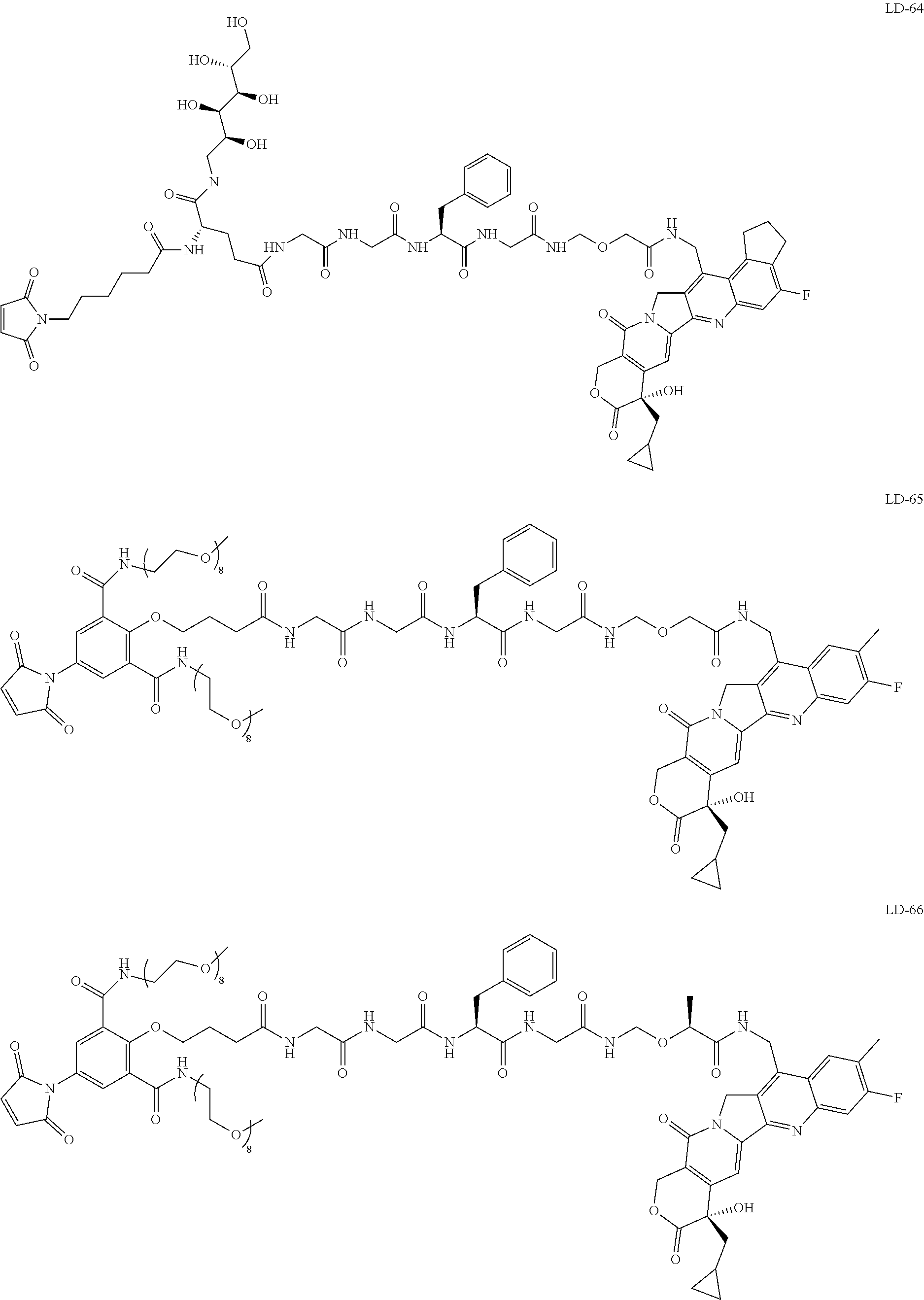

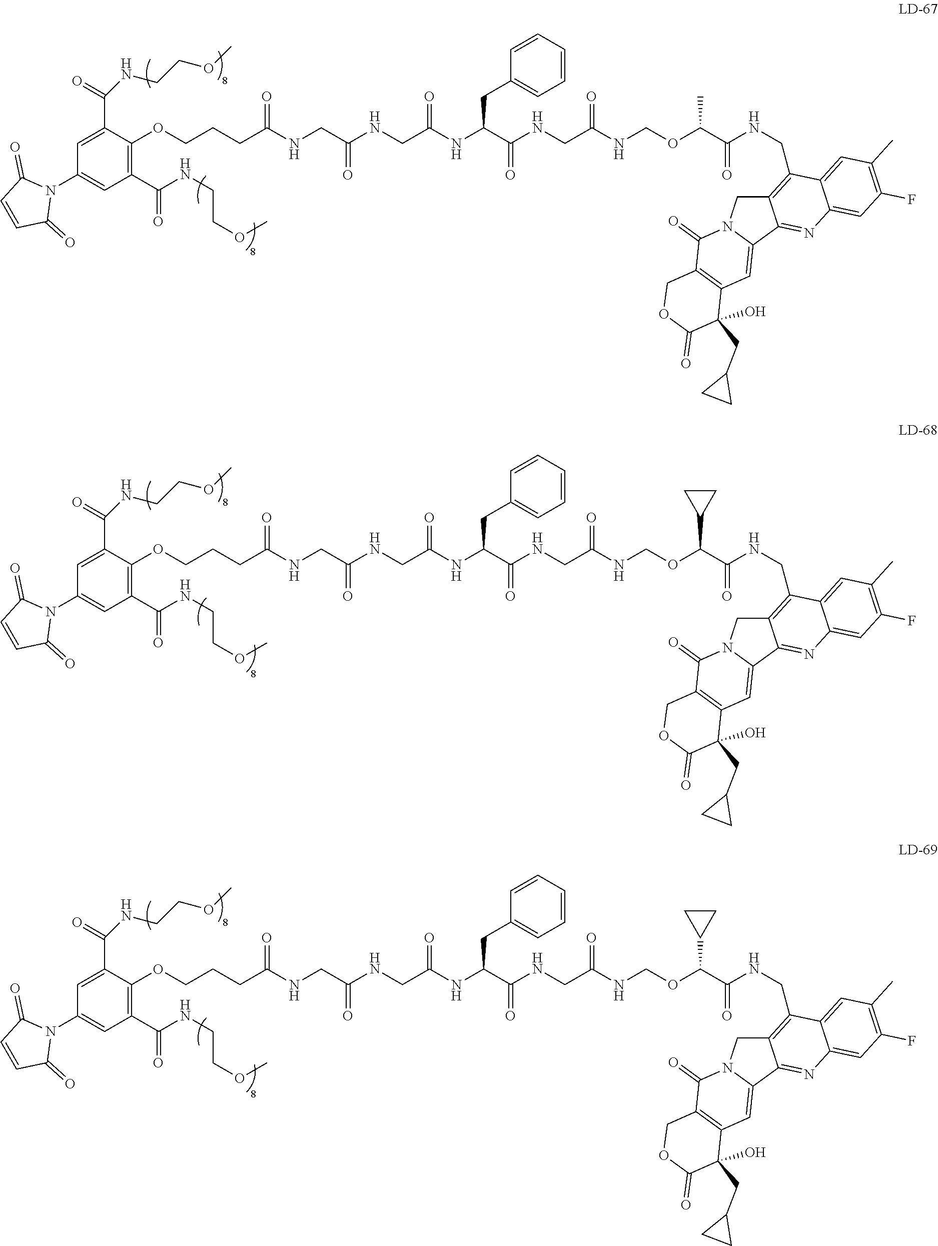

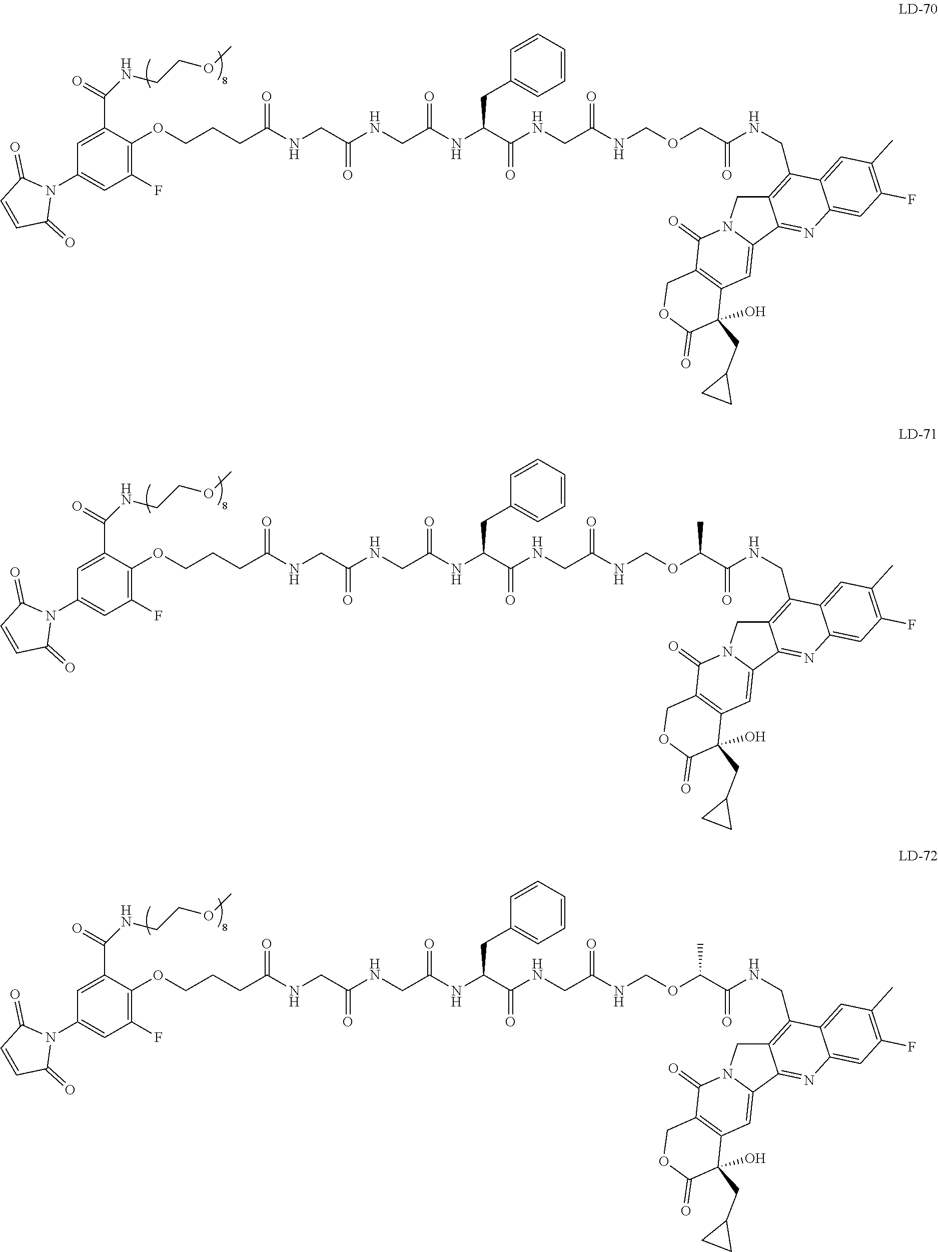

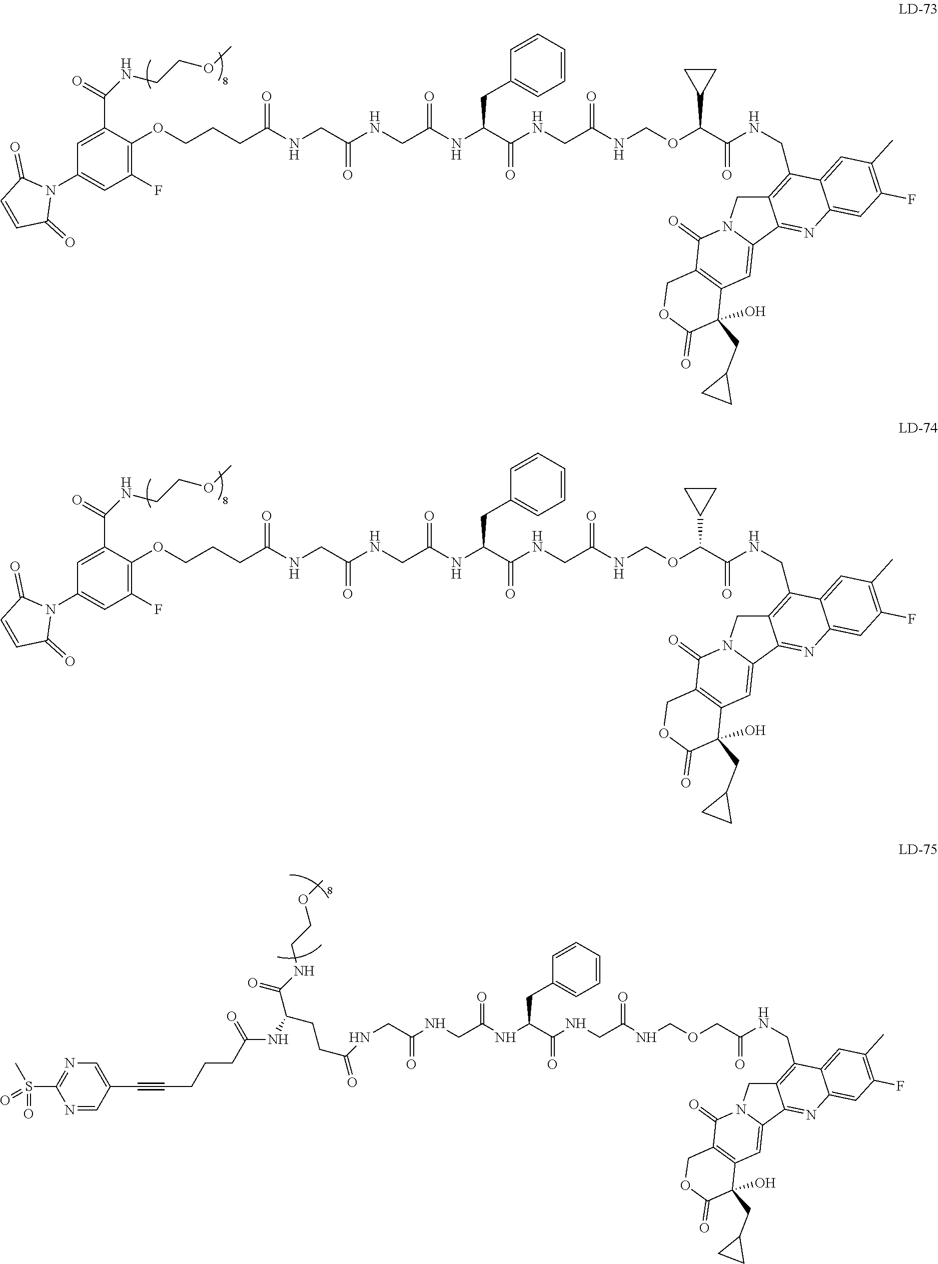

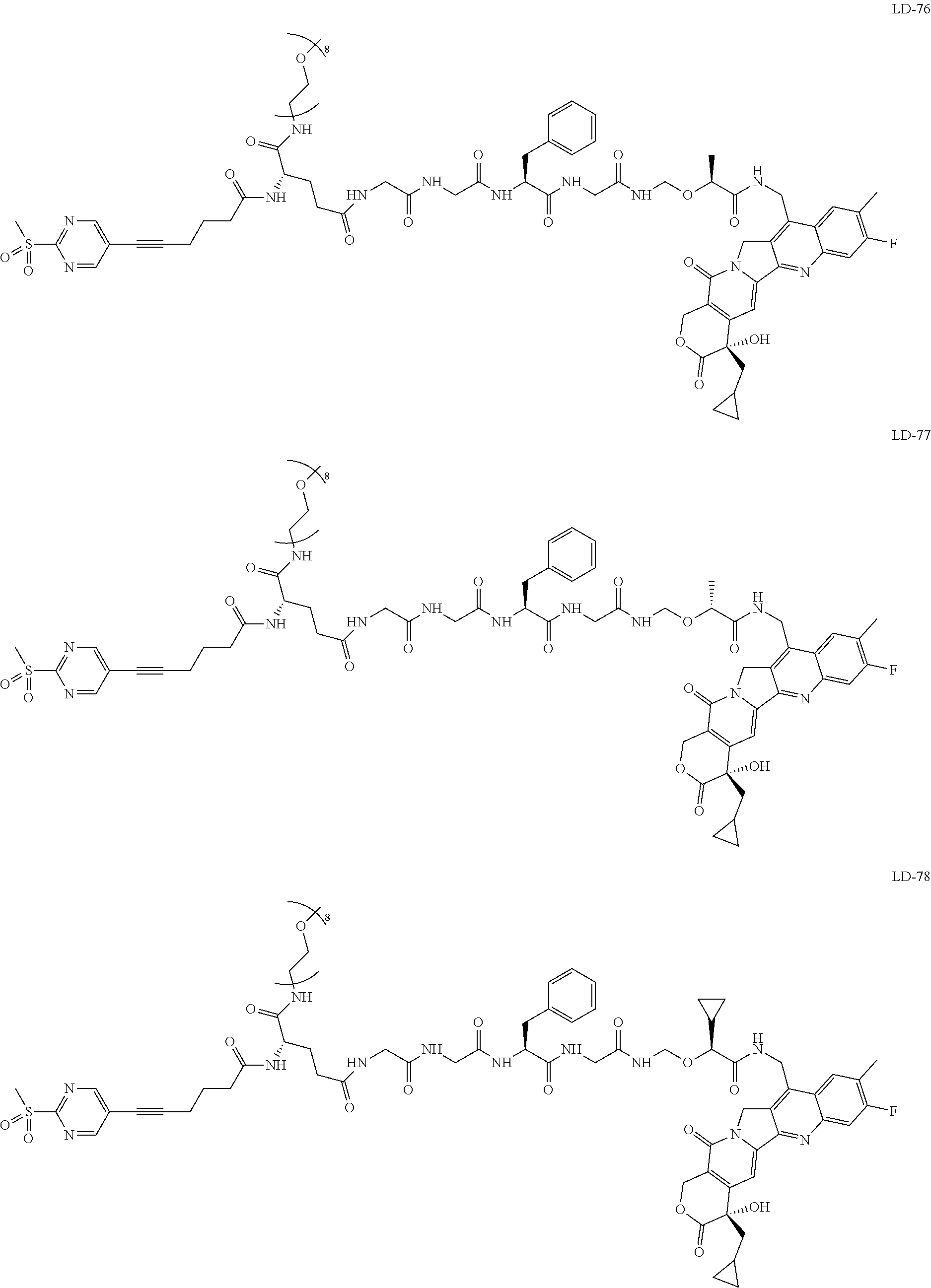

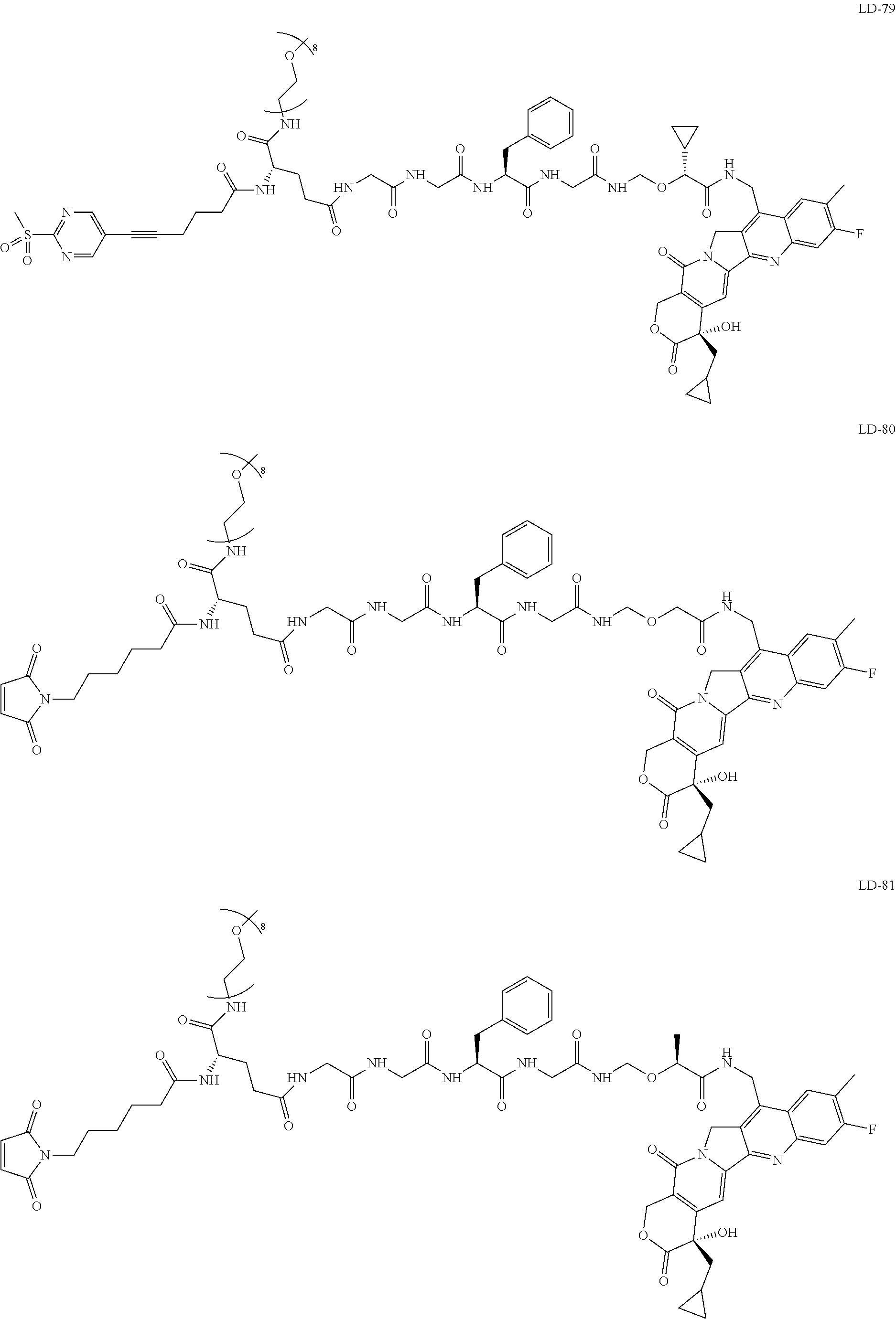

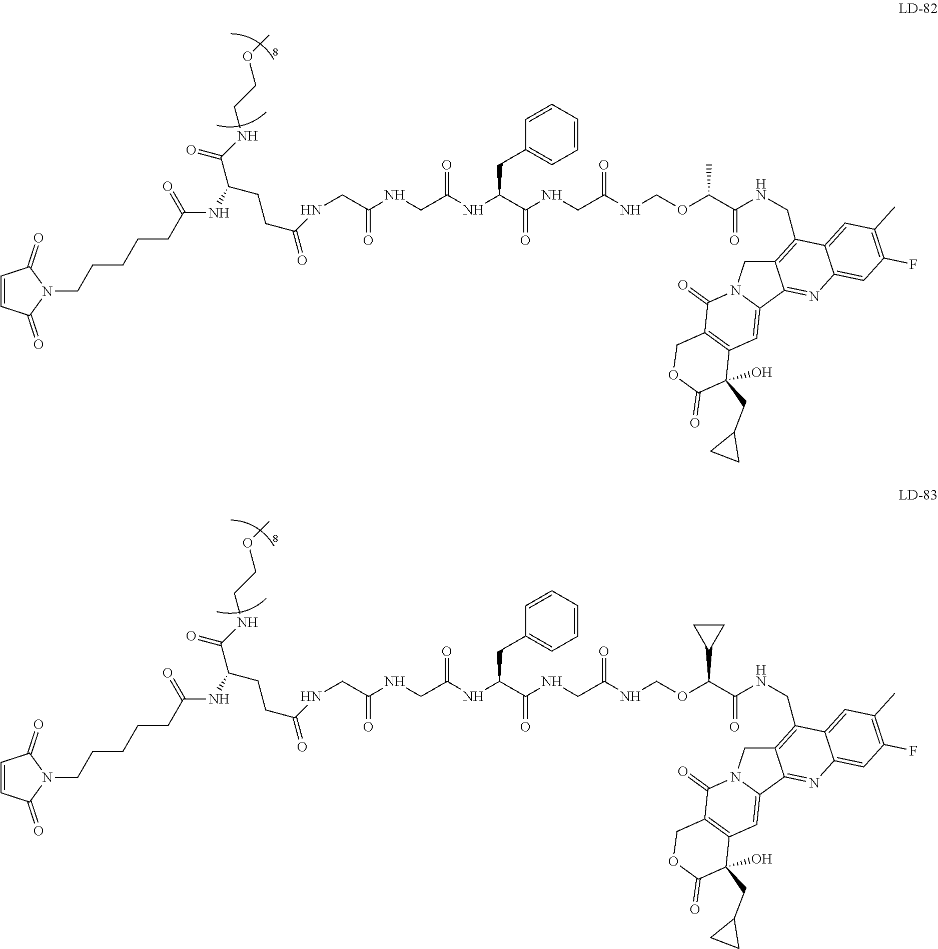

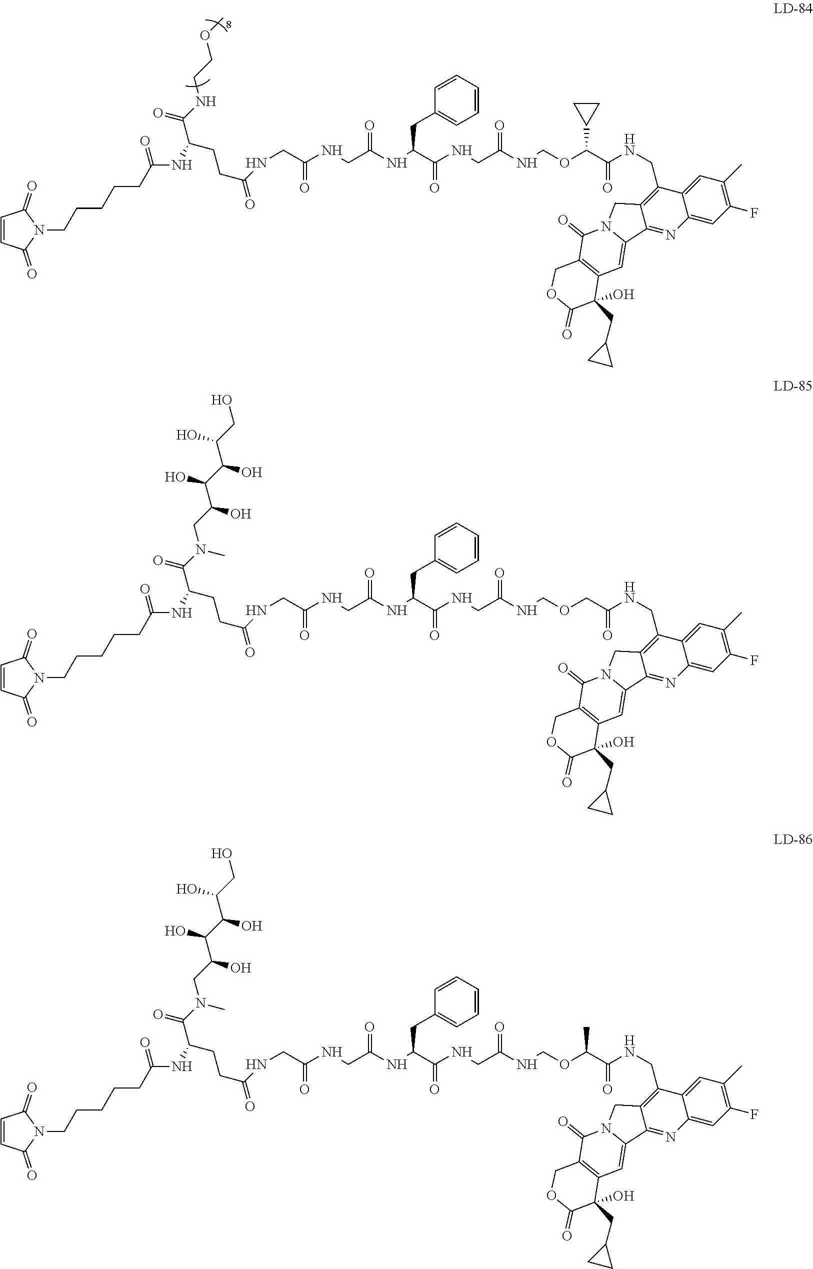

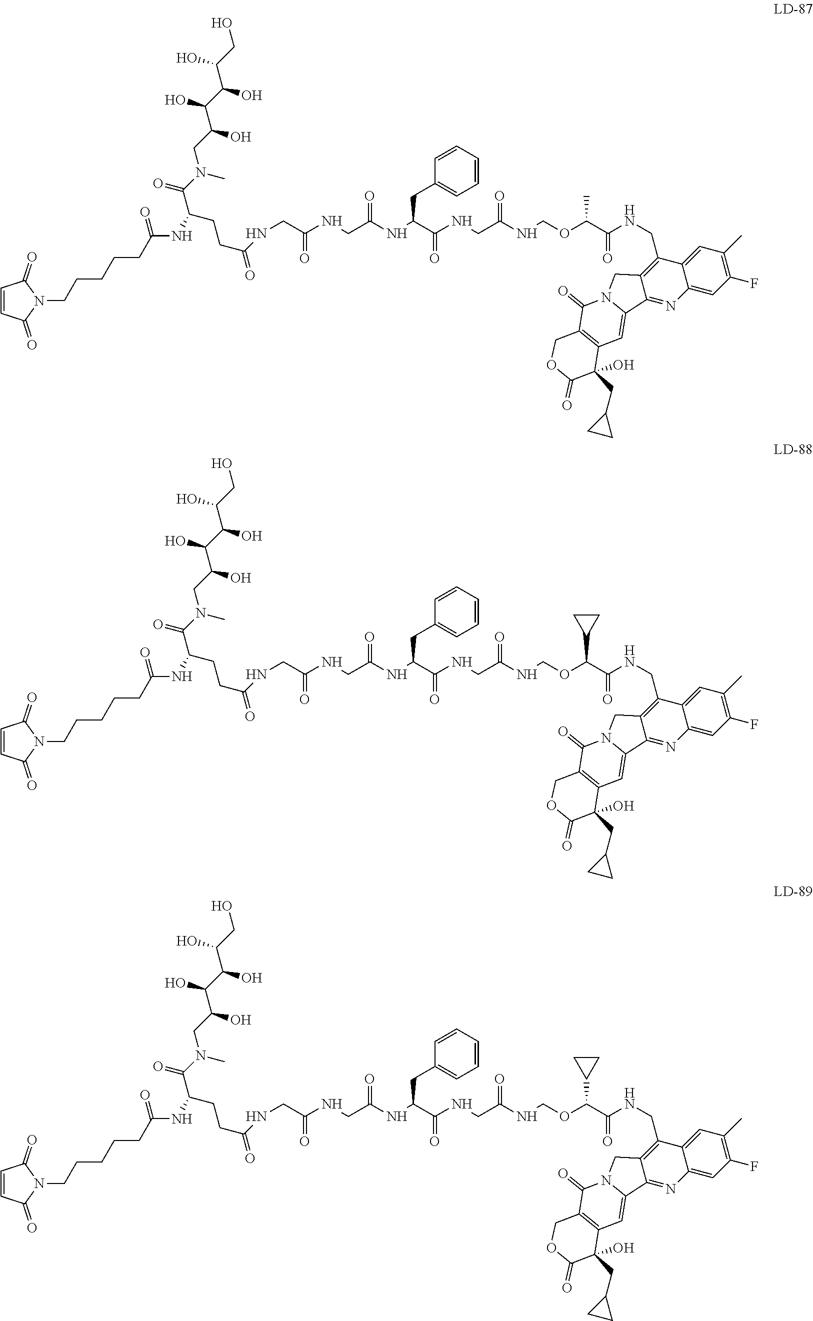

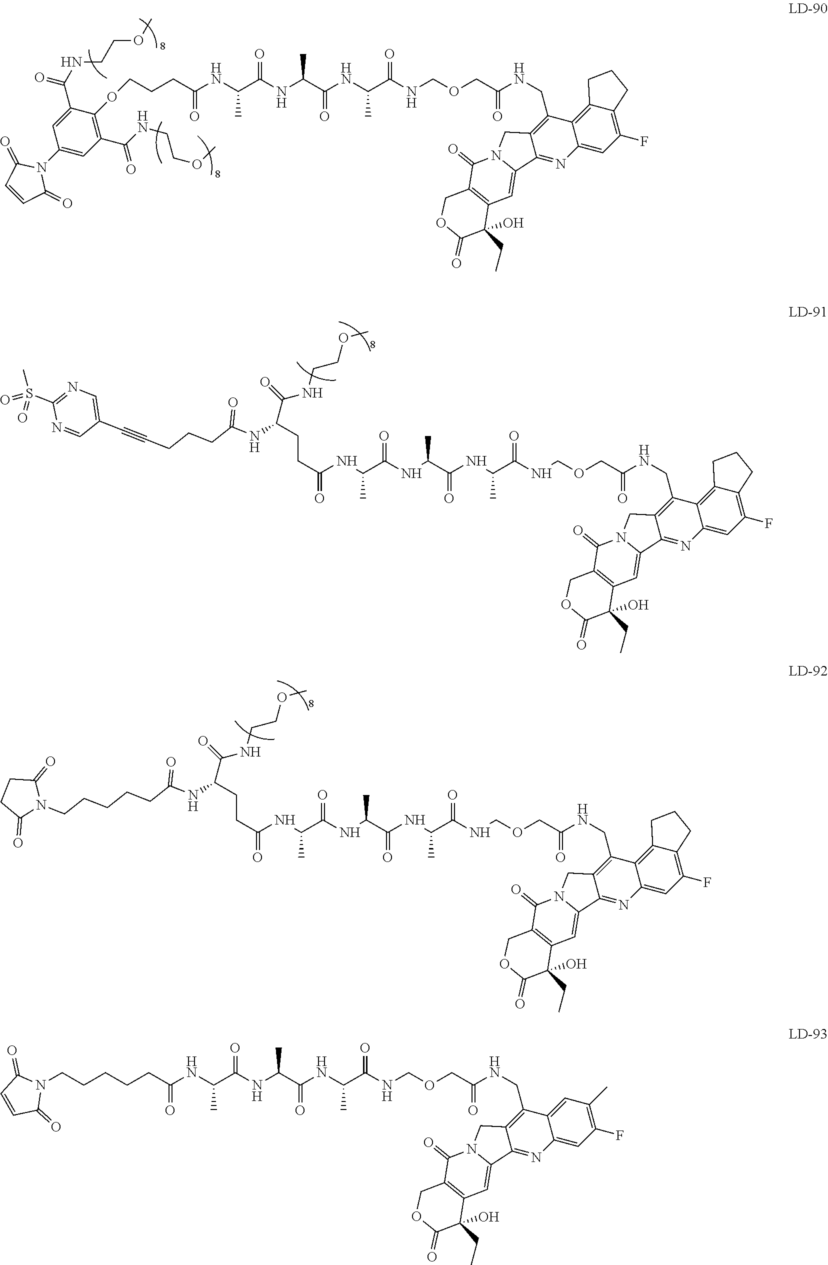

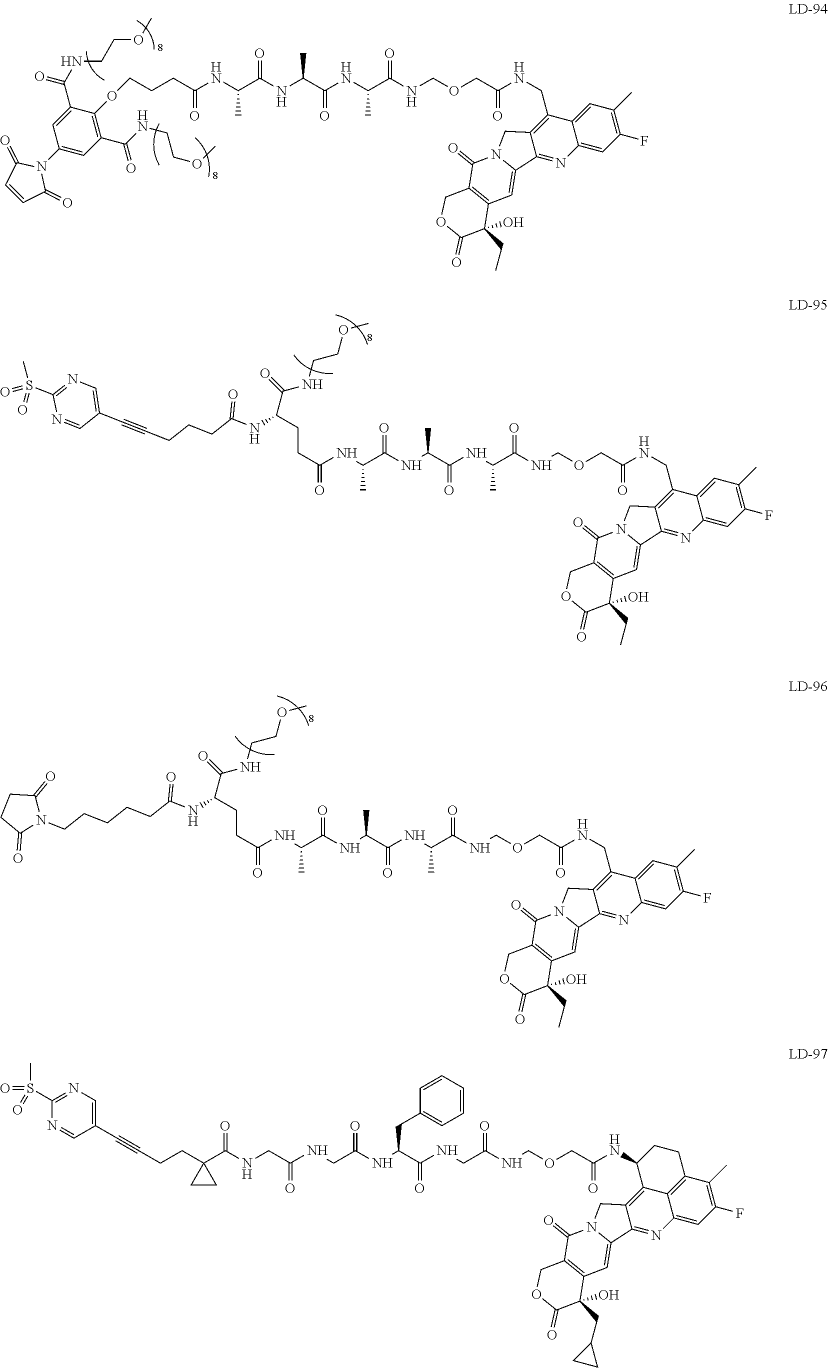

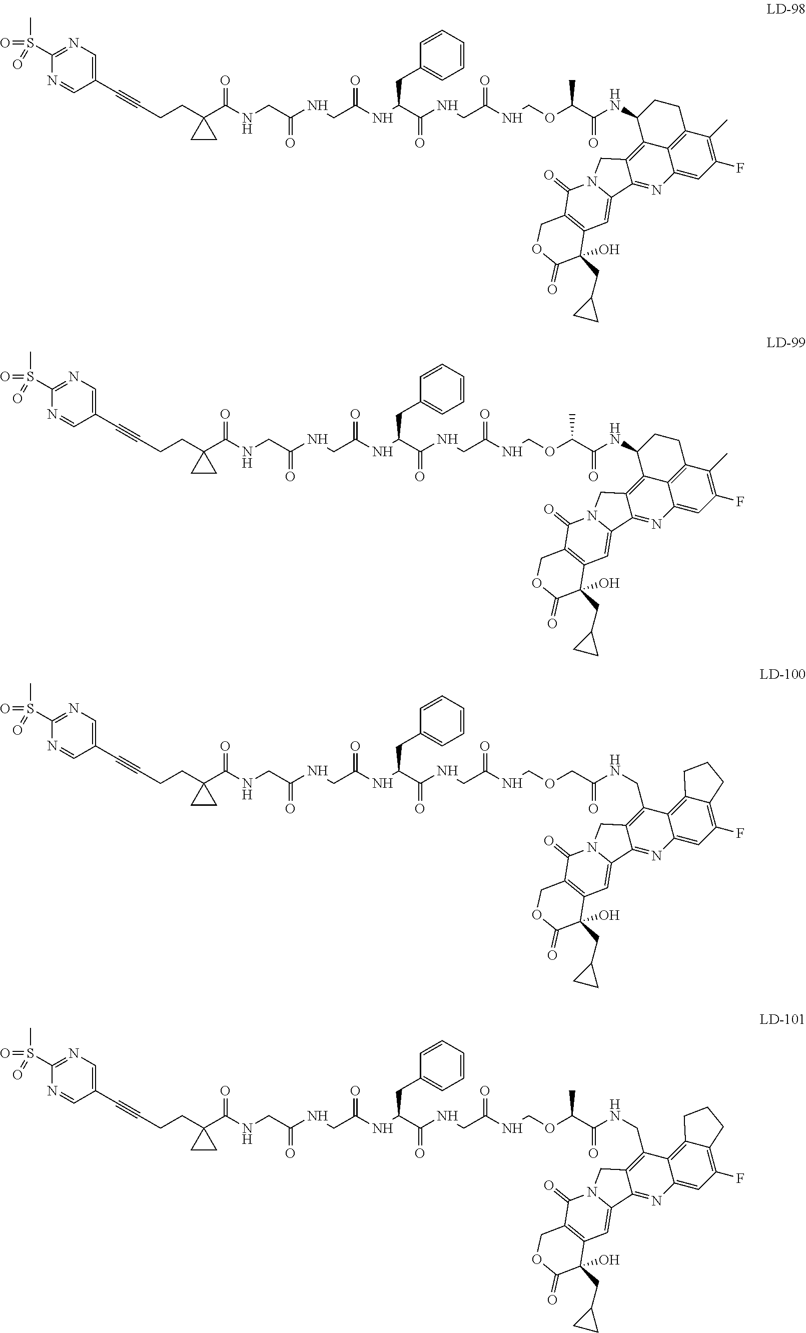

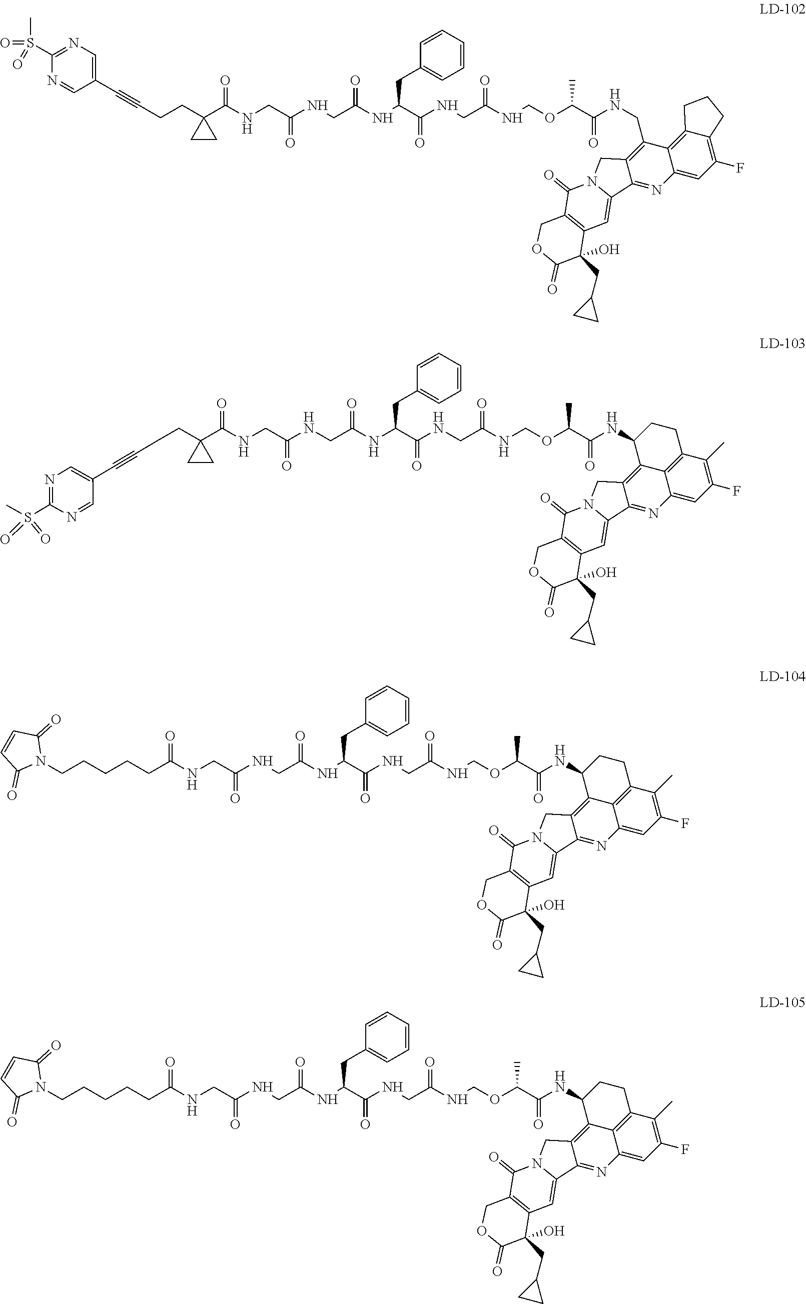

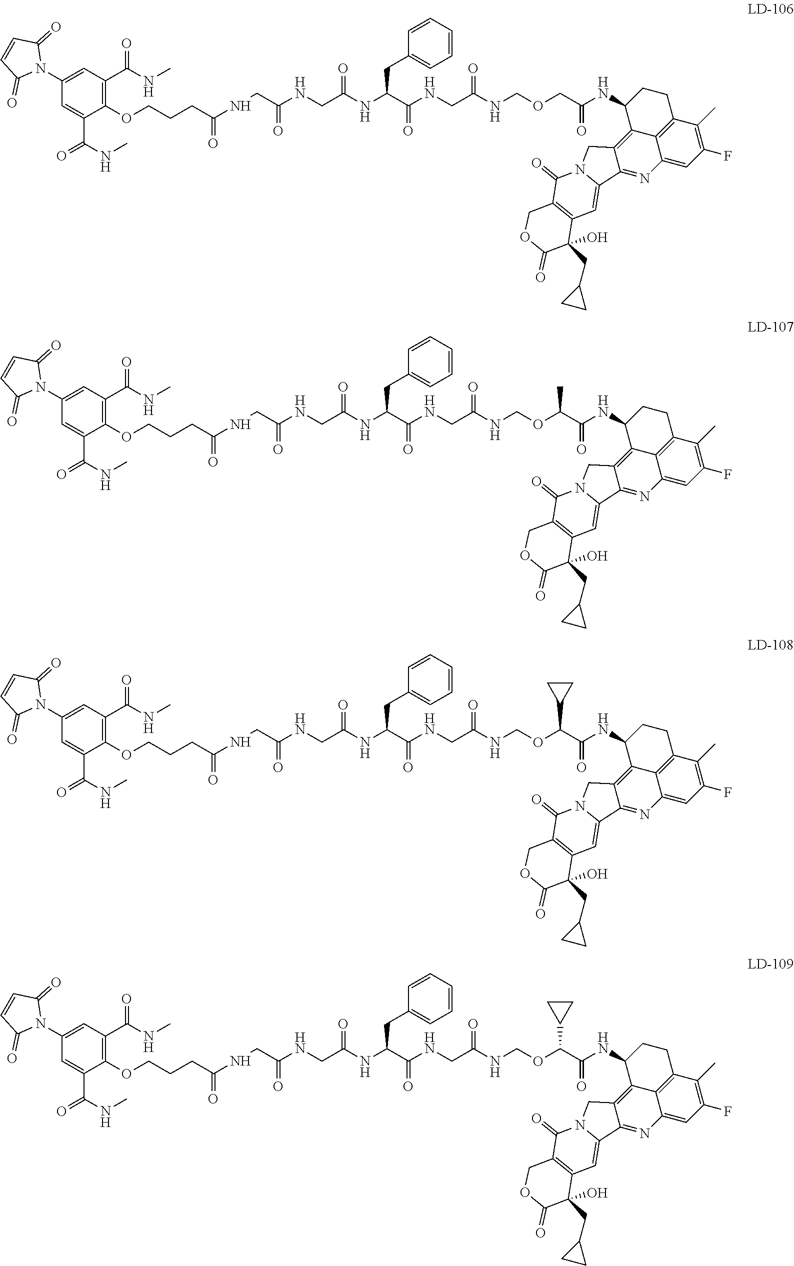

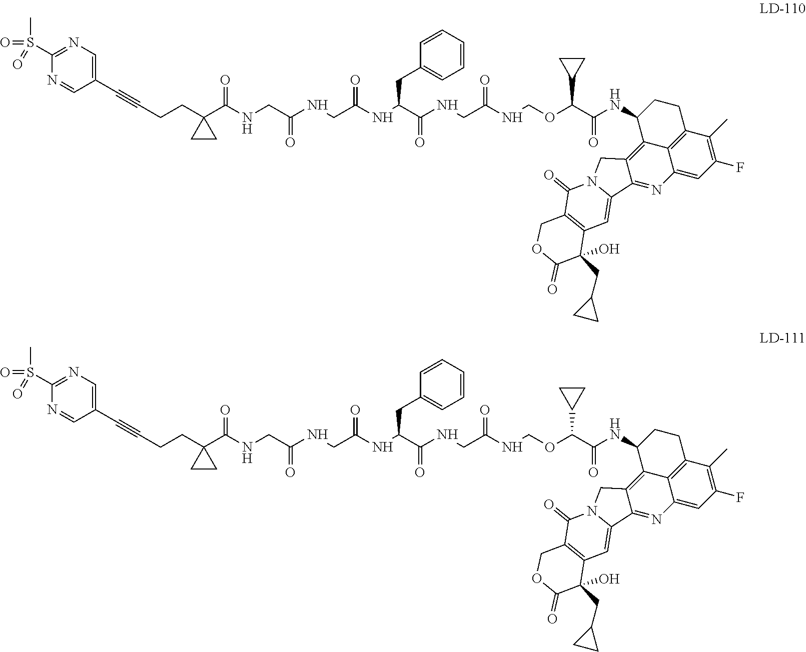 [SMILES: Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1 ; Cc1c(F)cc2nc3c(c4c2c1CC[C@@H]4NC(=O)[C@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1]

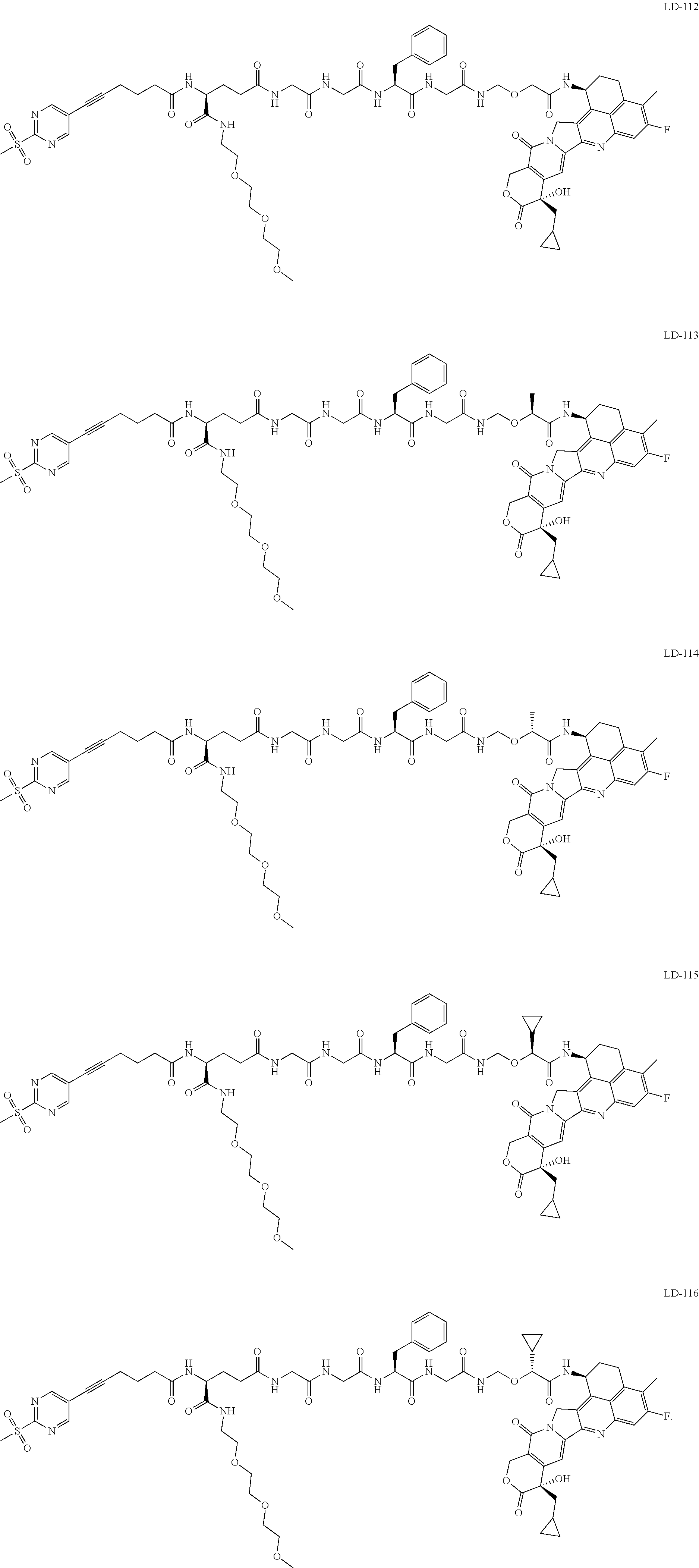

According to the embodiments of the present disclosure, the example of the conjugate may be selected from one of the followings, wherein:
y represents the average number of small molecule drugs attached to each monoclonal antibody (DAR), which can be selected from an integer or decimal, such as an integer or decimal selected from 1 to 50, an integer or decimal selected from 1 to 20, or an integer or decimal selected from 1 to 10:

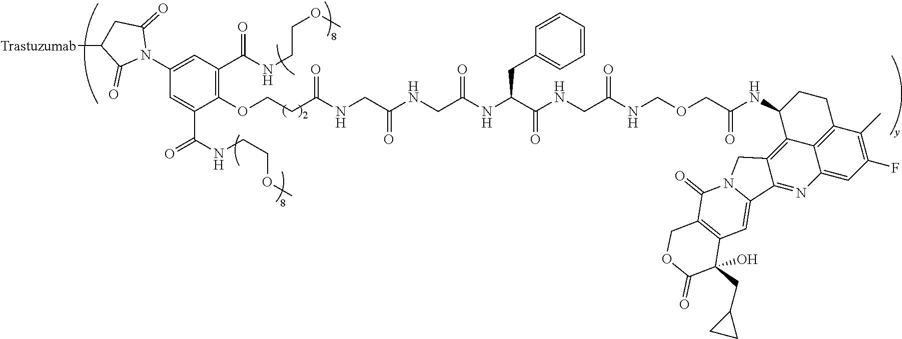

FADC-1A

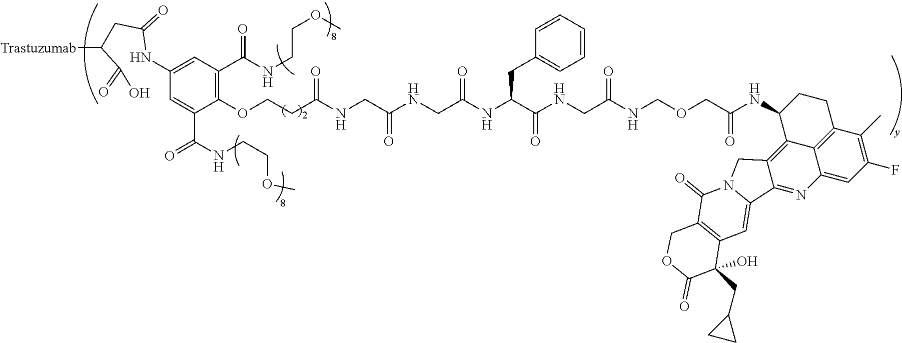

FADC-1B

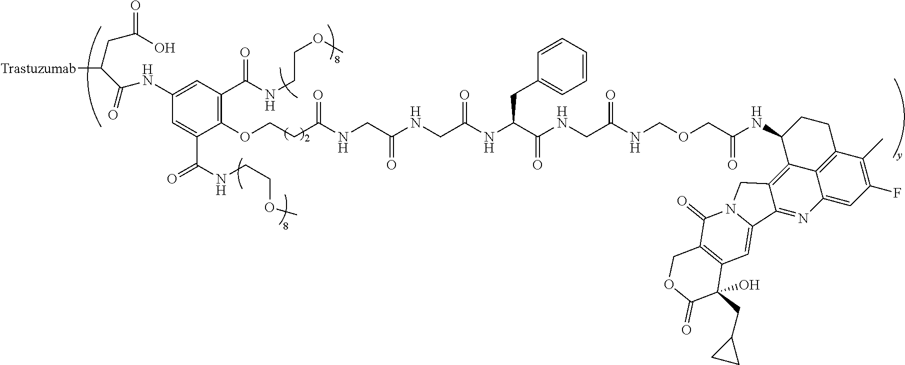

FADC-1C

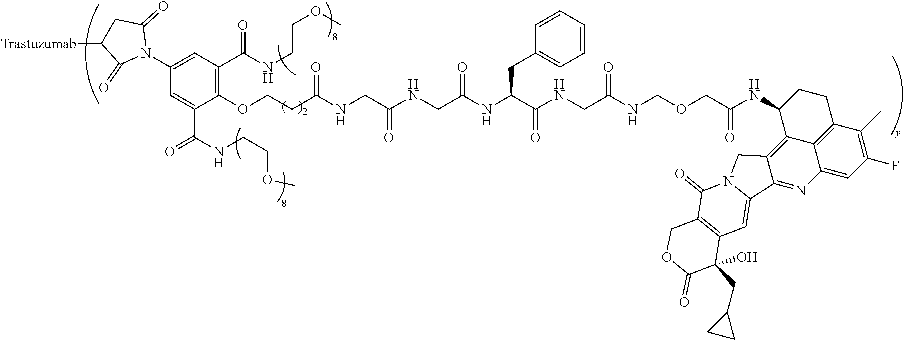

FADC-X₁A

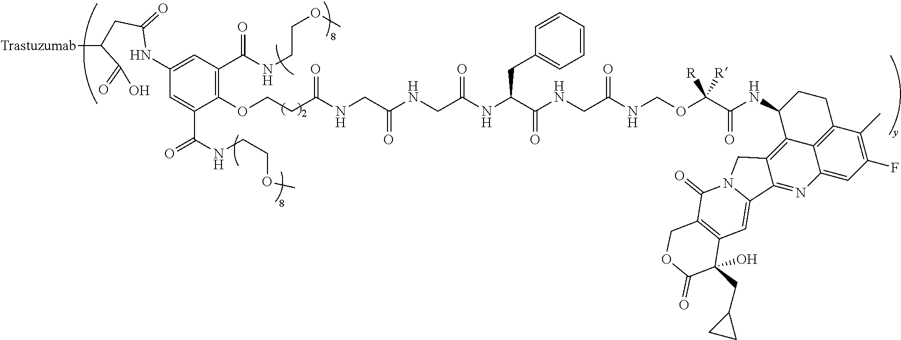

FADC-X₁B

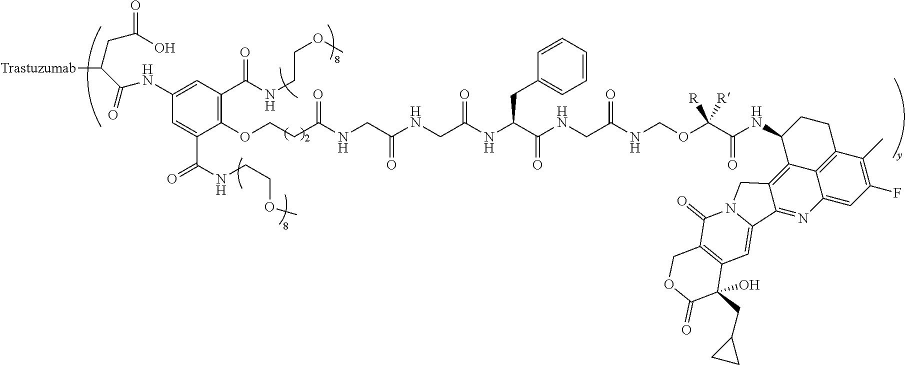

FADC-X₁C
wherein, R is selected from —CH₃, R′ is selected from —H;
R is selected from —H, R′ is selected from —CH₃;

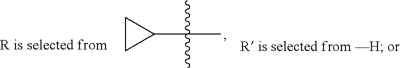 [SMILES: *C1CC1]

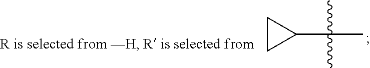 [SMILES: *C1CC1]

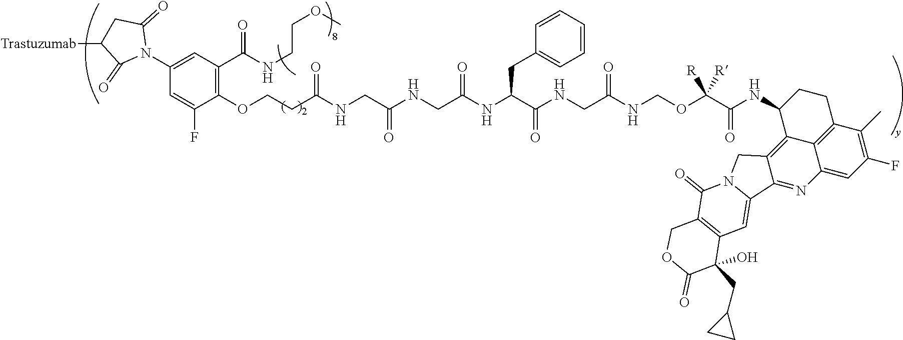

FADC-X₂A

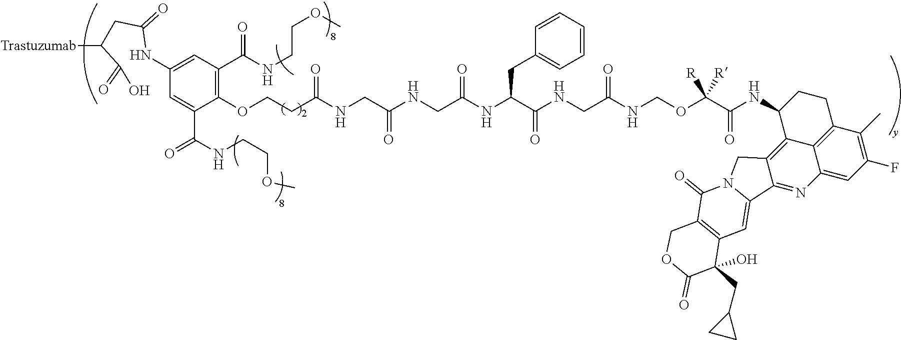

FADC-X₂B

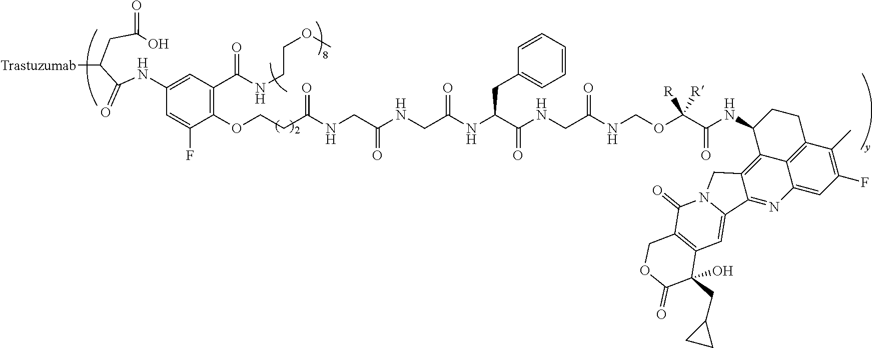

FADC-X₂C
wherein, R is selected from —H, R′ is selected from —H;
R is selected from —CH₃, R′ is selected from —H;
R is selected from —H, R′ is selected from —CH₃;

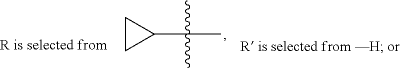 [SMILES: *C1CC1]

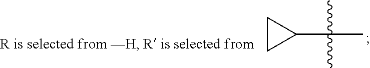 [SMILES: *C1CC1]

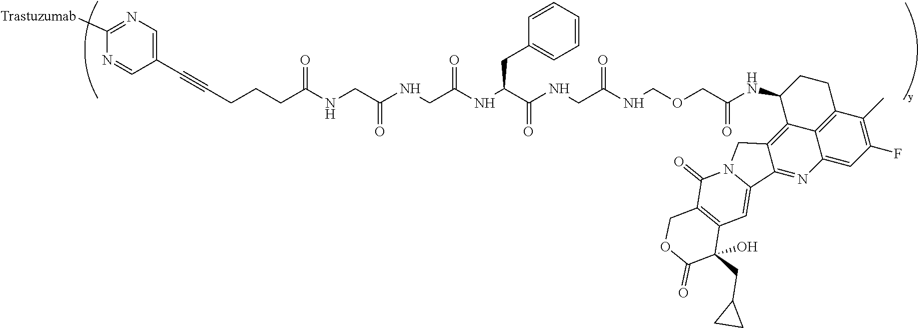

FADC-11

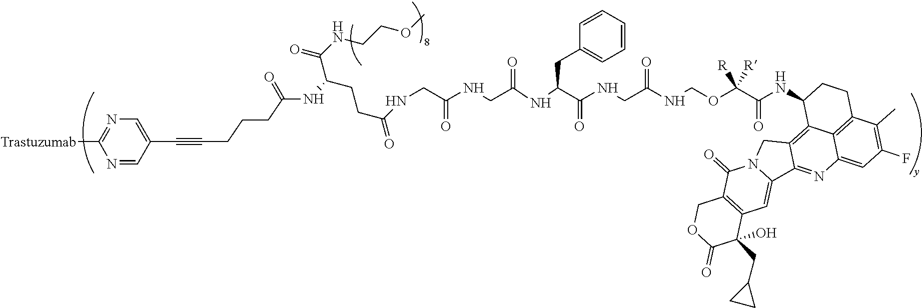

FADC-X₃
wherein, R is selected from —H, R′ is selected from —H;
R is selected from —CH₃, R′ is selected from —H;
R is selected from —H, R′ is selected from —CH₃;

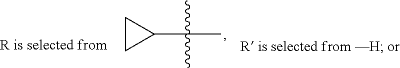 [SMILES: *C1CC1]

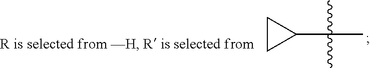 [SMILES: *C1CC1]

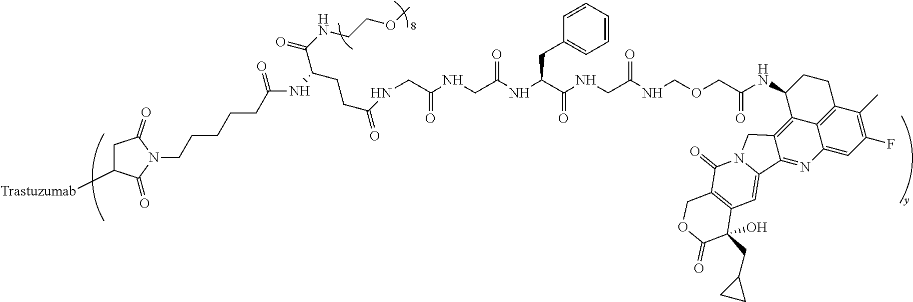

FADC-17A

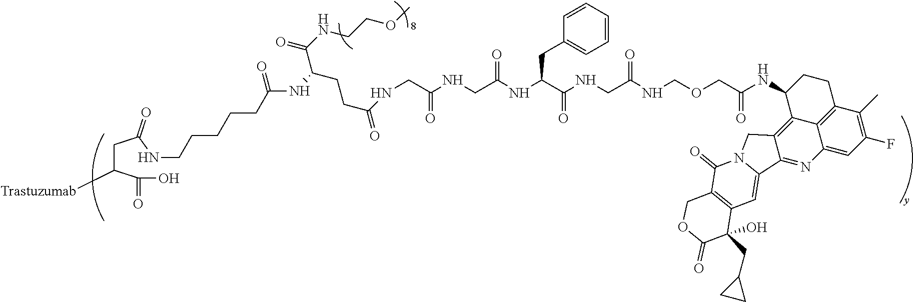

FADC-17B

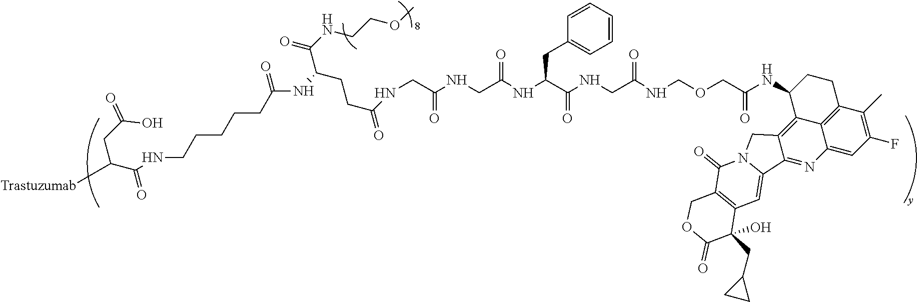

FADC-17C

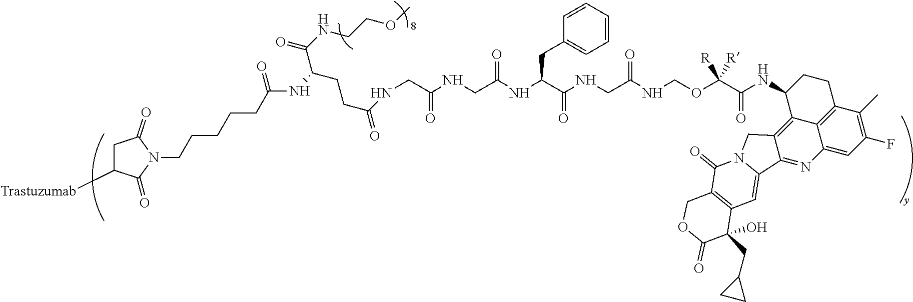

FADC-X₄A

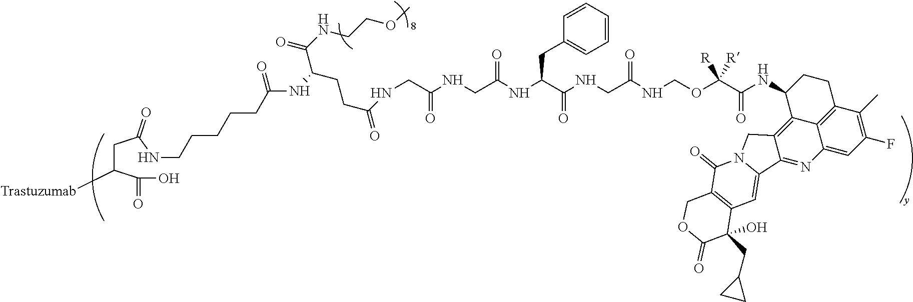

FADC-X₄B

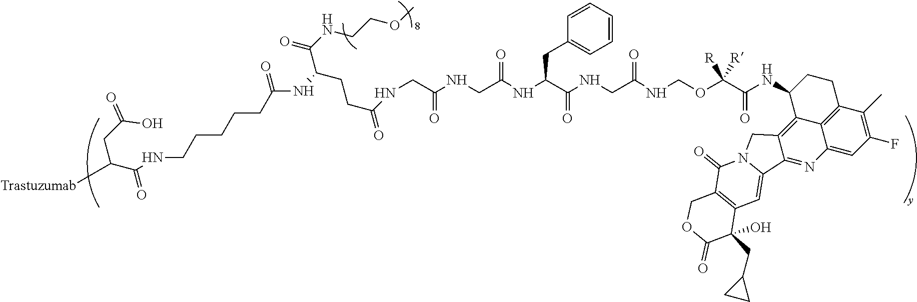

FADC-X₄C
wherein, R is selected from —CH₃, R′ is selected from —H;
R is selected from —H, R′ is selected from —CH₃;

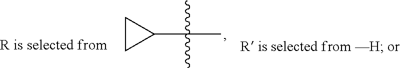 [SMILES: *C1CC1]

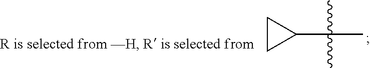 [SMILES: *C1CC1]

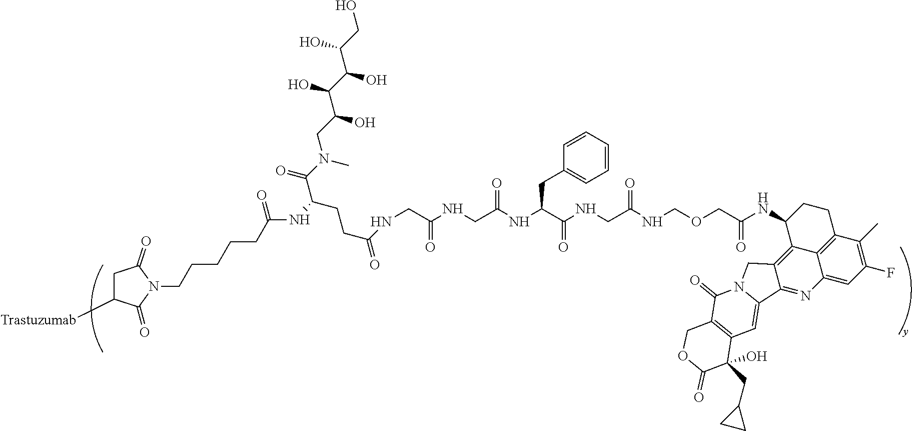

FADC-22A

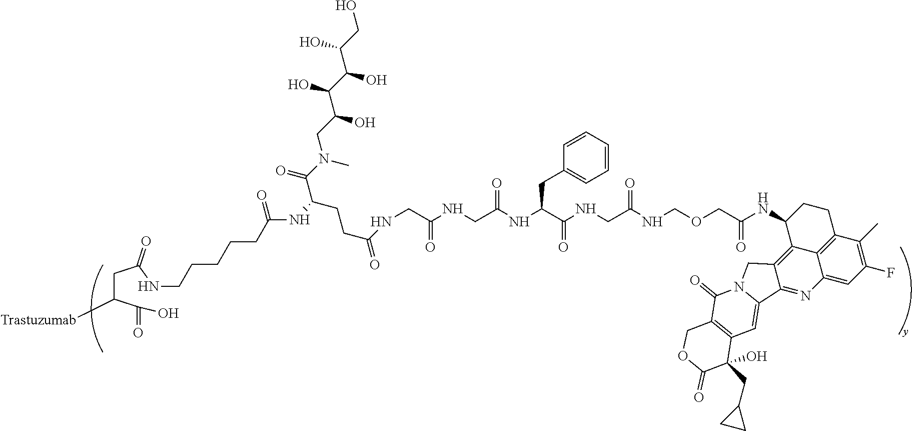

FADC-22B

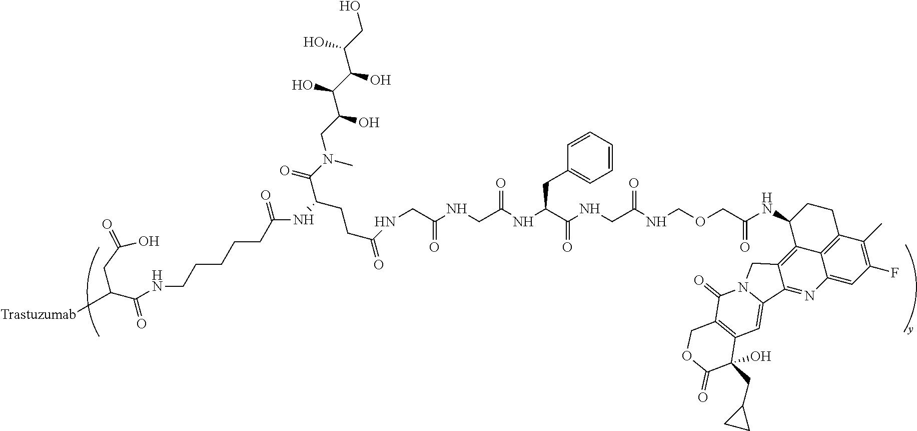

FADC-22C

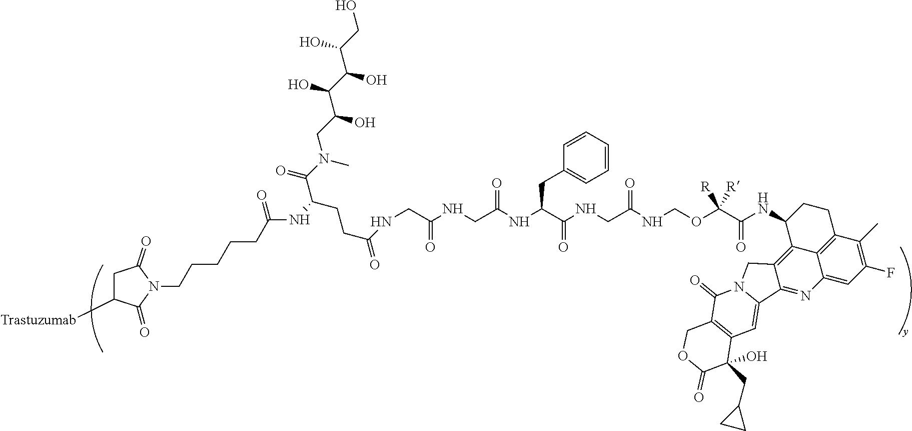

FADC-X₅A

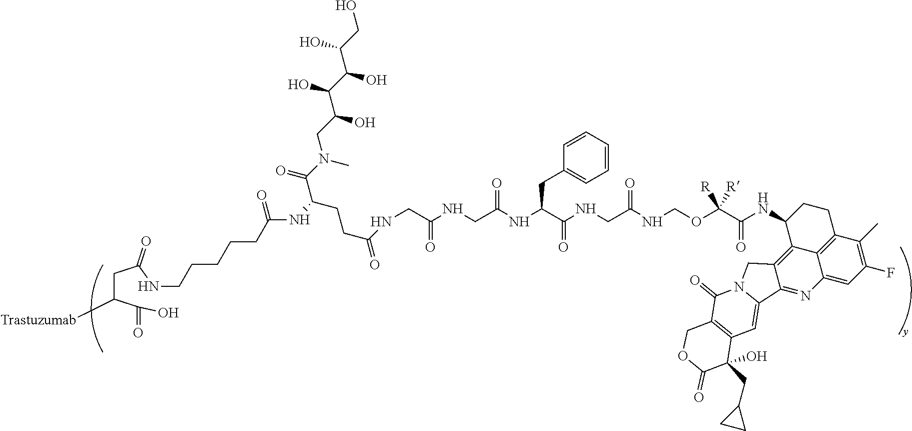

FADC-X₅B

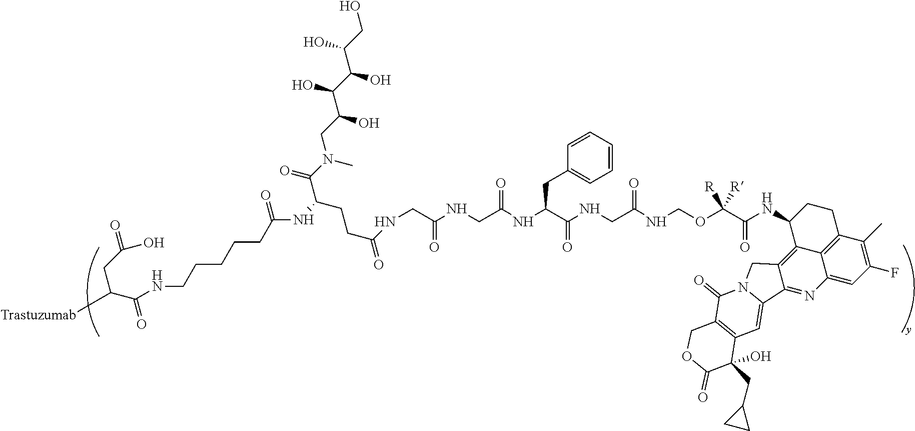

FADC-X₅C
wherein, R is selected from —CH₃, R′ is selected from —H;
R is selected from —H, R′ is selected from —CH₃;

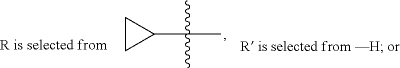 [SMILES: *C1CC1]

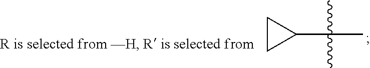 [SMILES: *C1CC1]

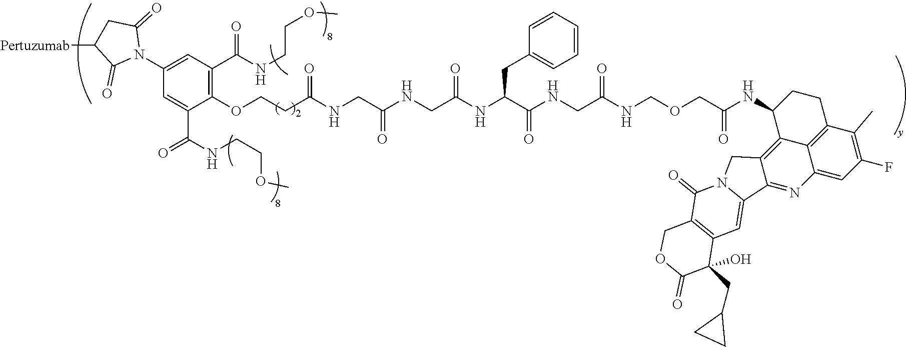

FADC-27A

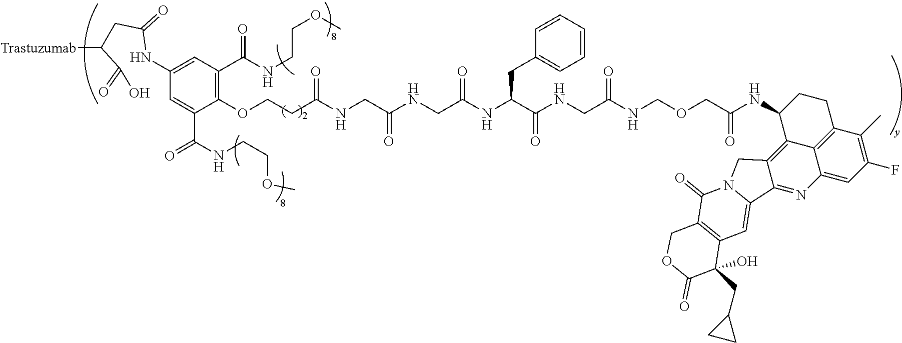

FADC-27B

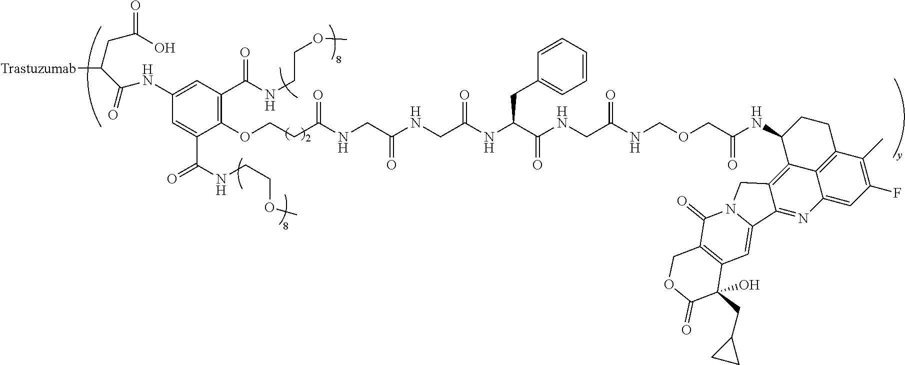

FADC-27C

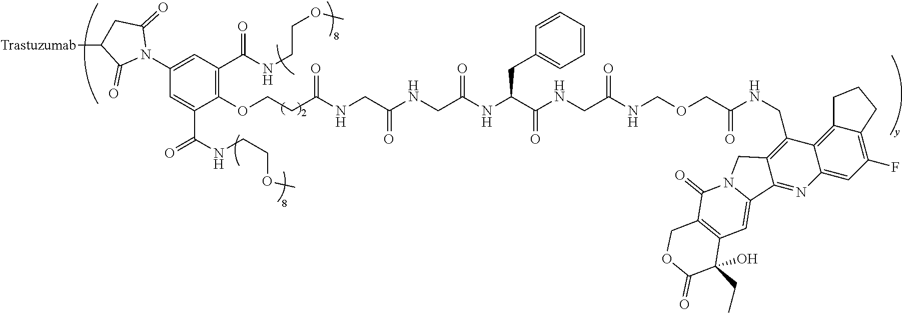

FADC-28A

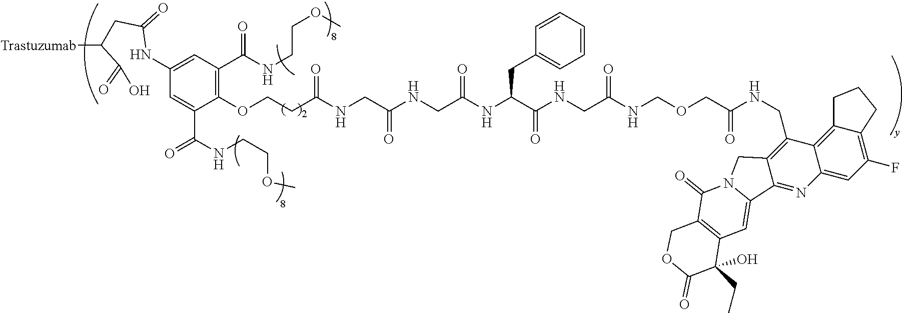

FADC-28B

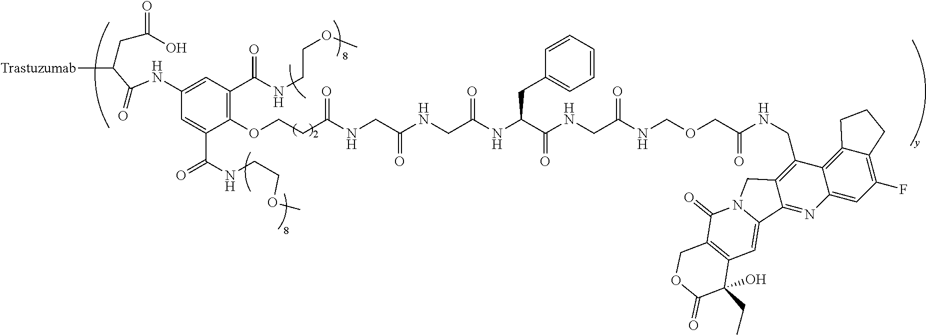

FADC-28C

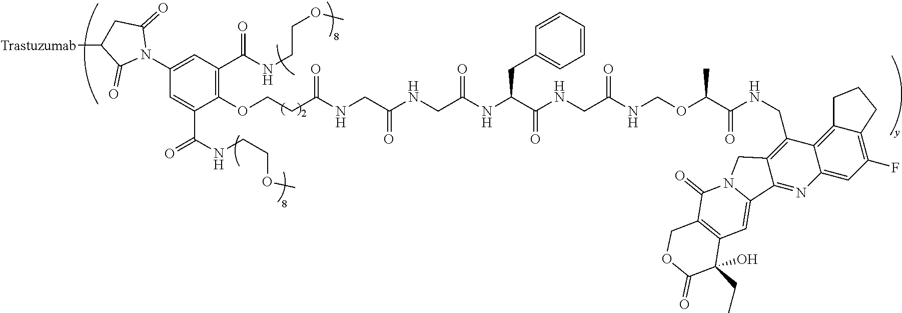

FADC-29A

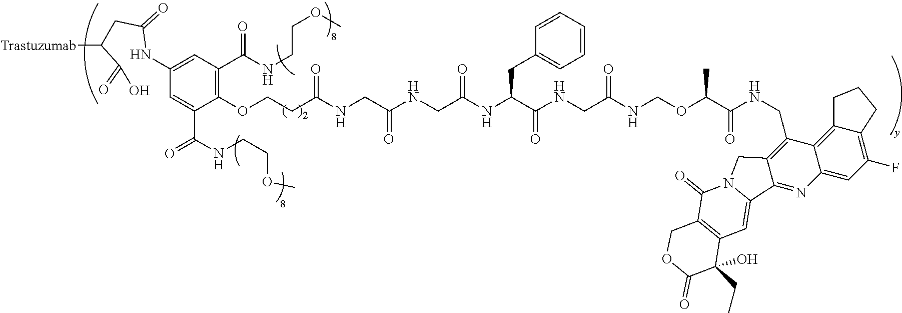

FADC-29B

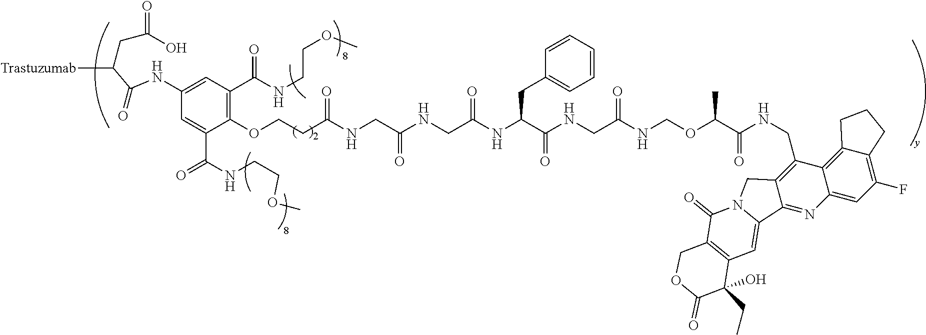

FADC-29C

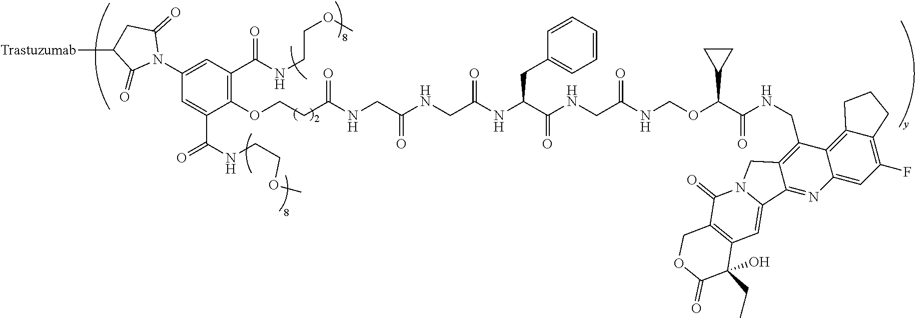

FADC-30A

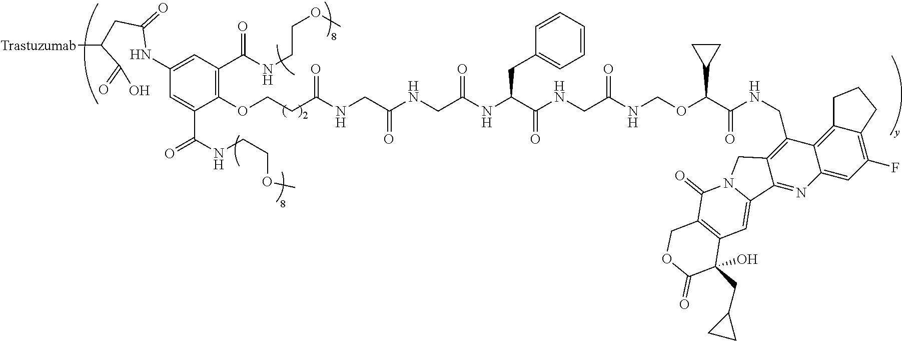

FADC-30B

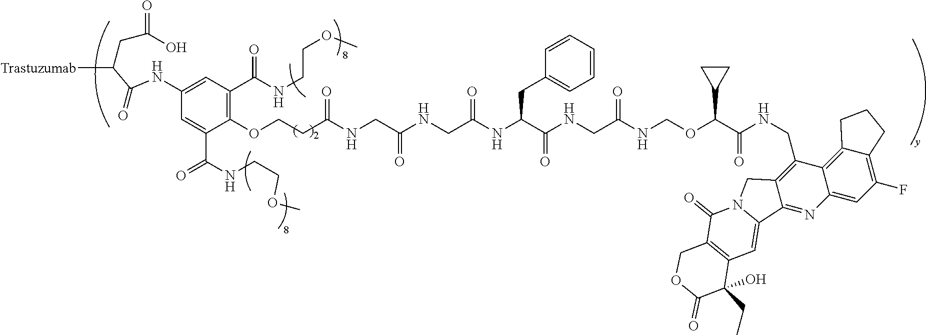

FADC-30C

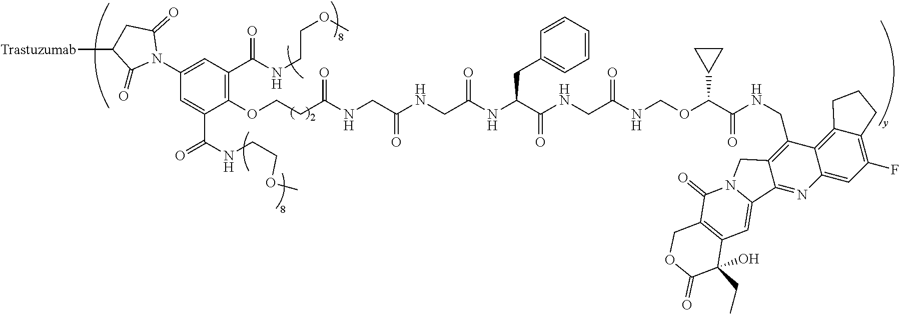

FADC-31A

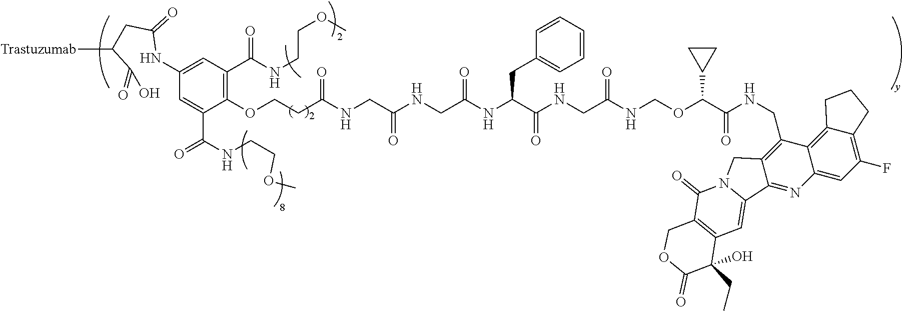

FADC-31B

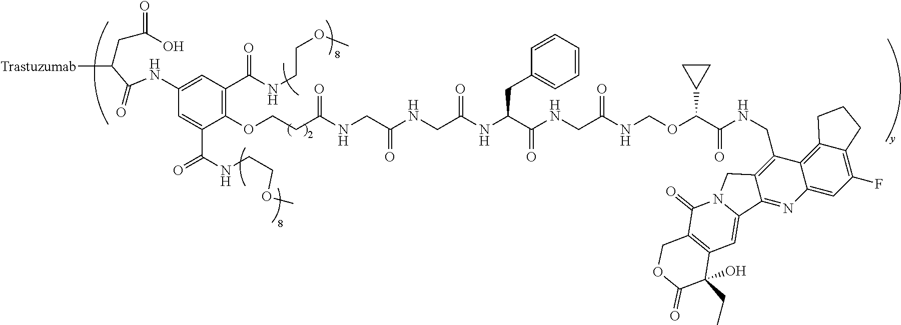

FADC-31C

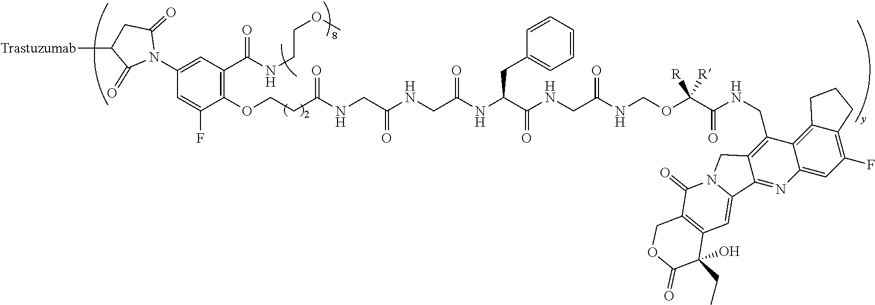

FADC-X₆A

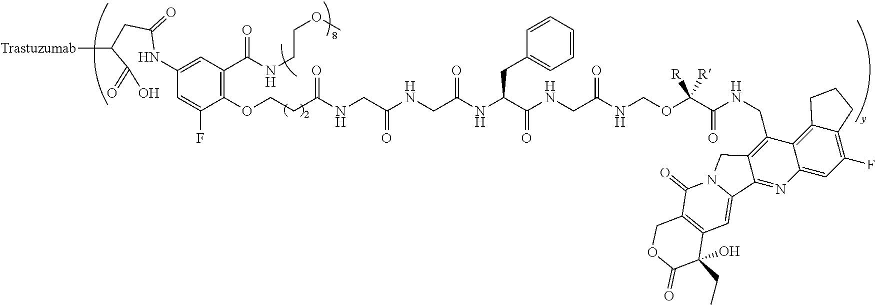

FADC-X₆B

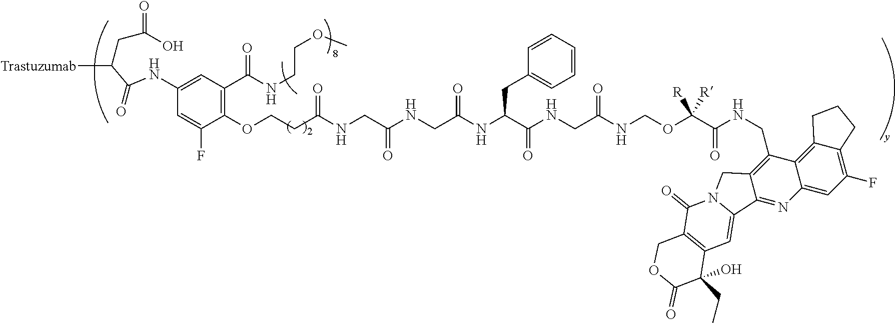

FADC-X₆C
wherein, R is selected from —H, R′ is selected from —H;
R is selected from —CH₃, R′ is selected from —H;

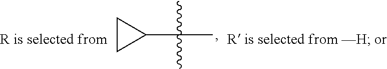 [SMILES: *C1CC1]

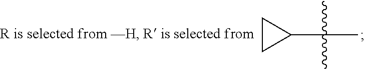 [SMILES: *C1CC1]

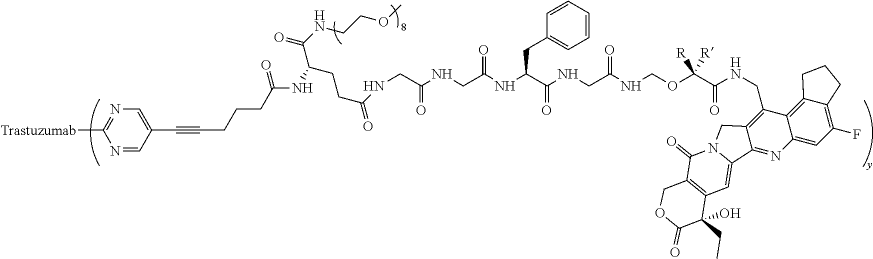

FADC-X₇
wherein, R is selected from —H, R′ is selected from —H;
R is selected from —CH₃, R′ is selected from —H; or

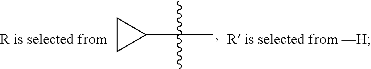 [SMILES: *C1CC1]

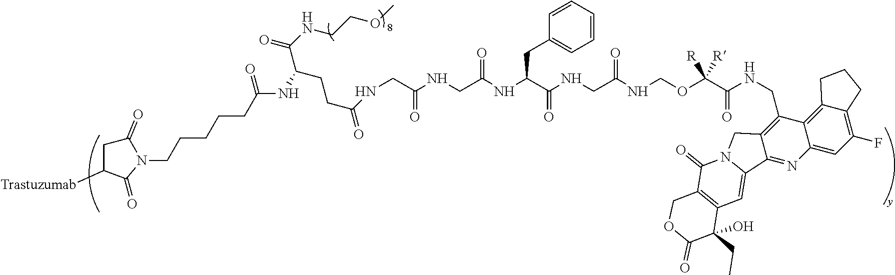

FADC-X₈A

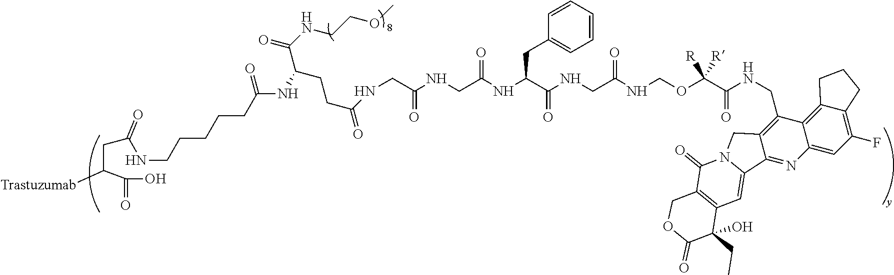

FADC-X₈B

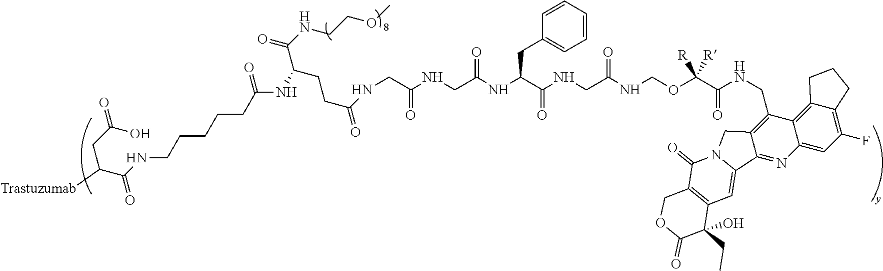

FADC-X₈C
wherein, R is selected from —H, R′ is selected from —H;
R is selected from —CH₃, R′ is selected from —H;

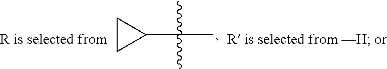 [SMILES: *C1CC1]

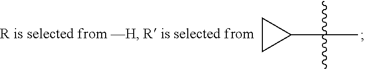 [SMILES: *C1CC1]

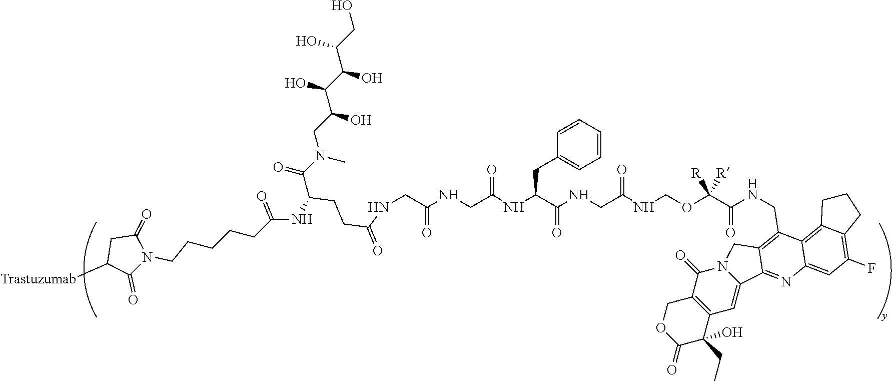

FADC-X₉A

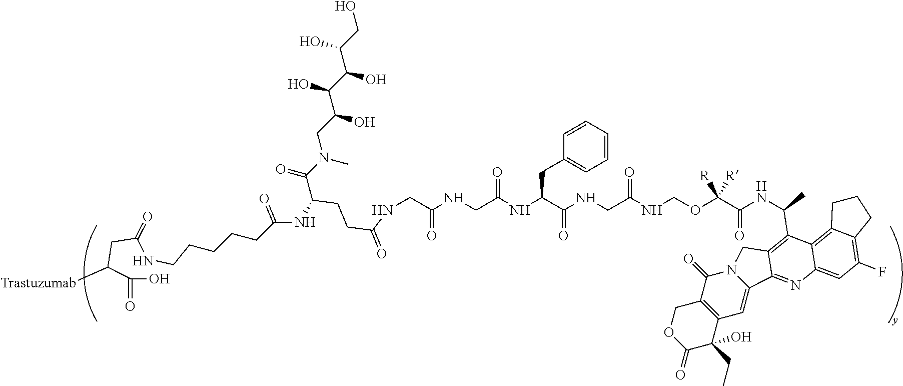

FADC-X₉B

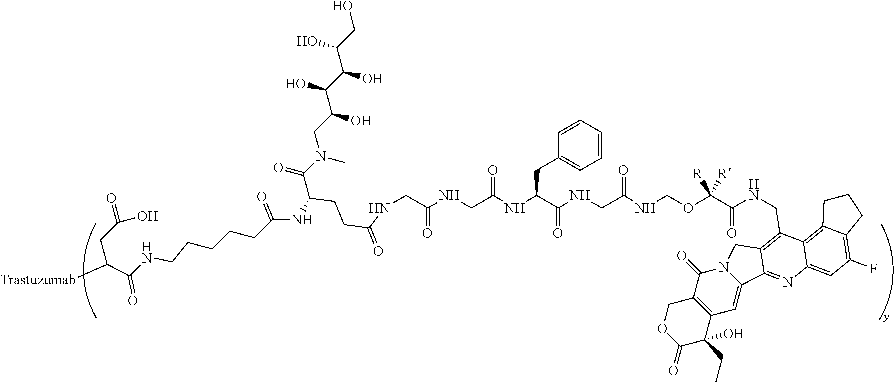

FADC-X₉C
wherein, R is selected from —H, R′ is selected from —H;
R is selected from —CH₃, R′ is selected from —H; or

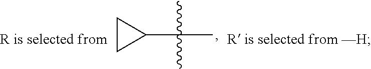 [SMILES: *C1CC1]

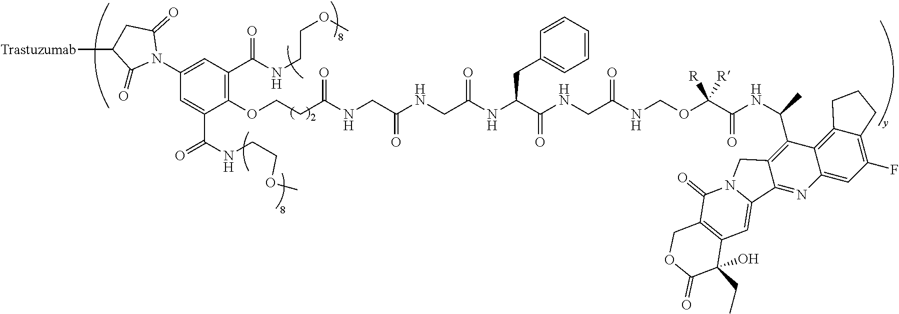

FADC-X₁₀A

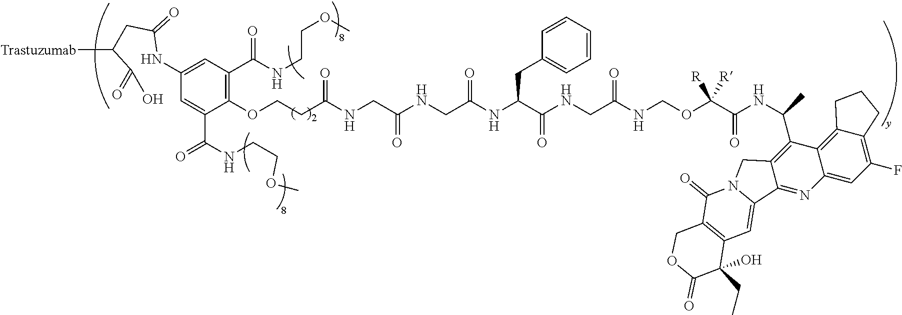

FADC-X₁₀B

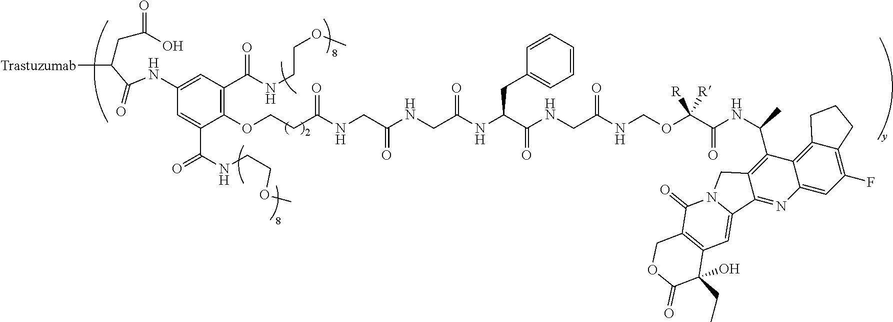

FADC-X₁₀C
wherein, R is selected from —H, R′ is selected from —H;

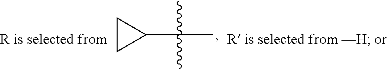 [SMILES: *C1CC1]

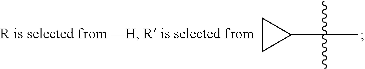 [SMILES: *C1CC1]

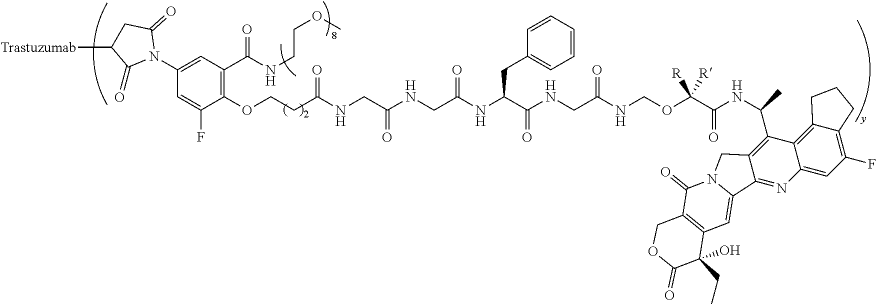

FADC-X₁₁A

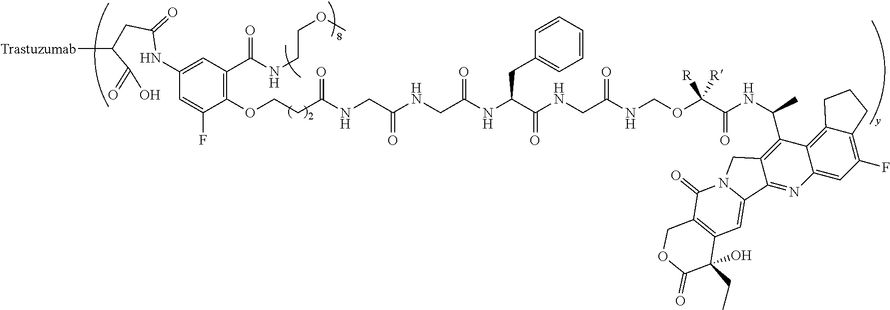

FADC-X₁₁B

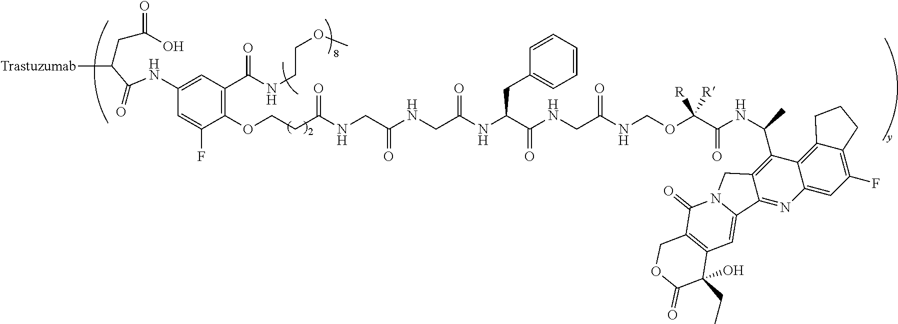

FADC-X₁₁C
wherein, R is selected from —H, R′ is selected from —H;

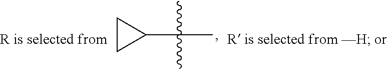 [SMILES: *C1CC1]

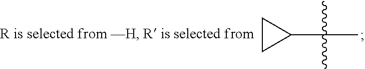 [SMILES: *C1CC1]

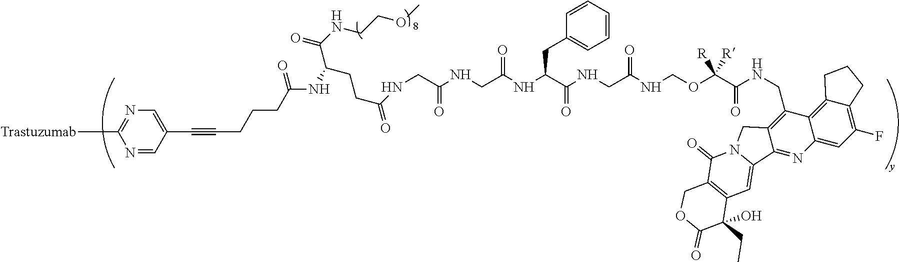

FADC-X₁₂
wherein, R is selected from —H, R′ is selected from —H;

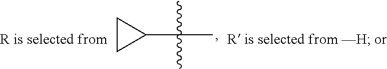 [SMILES: *C1CC1]

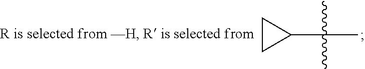 [SMILES: *C1CC1]

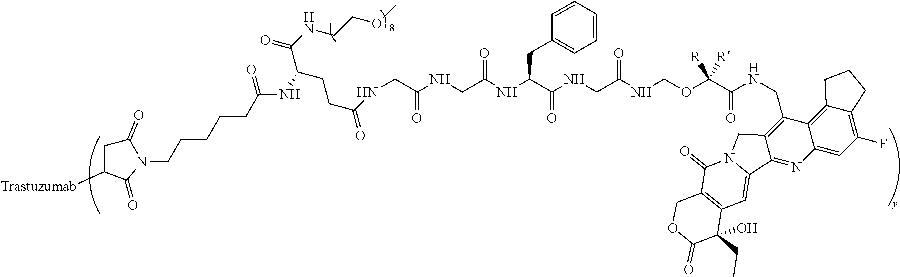

FADC-X₁₃A

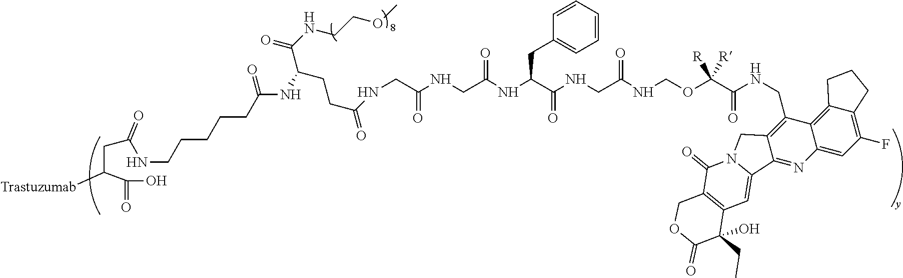

FADC-X₁₃B

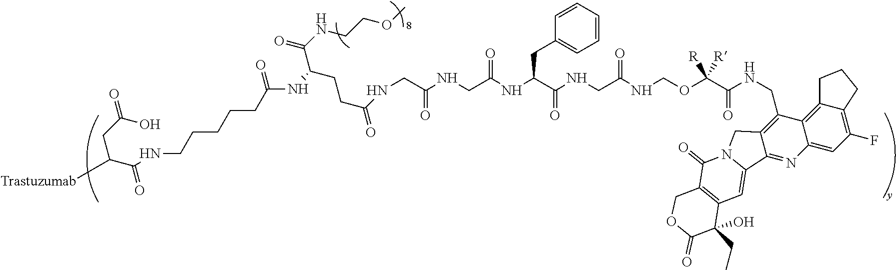

FADC-X₁₃C
wherein, R is selected from —H, R′ is selected from —H;

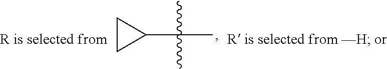 [SMILES: *C1CC1]

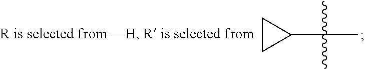 [SMILES: *C1CC1]

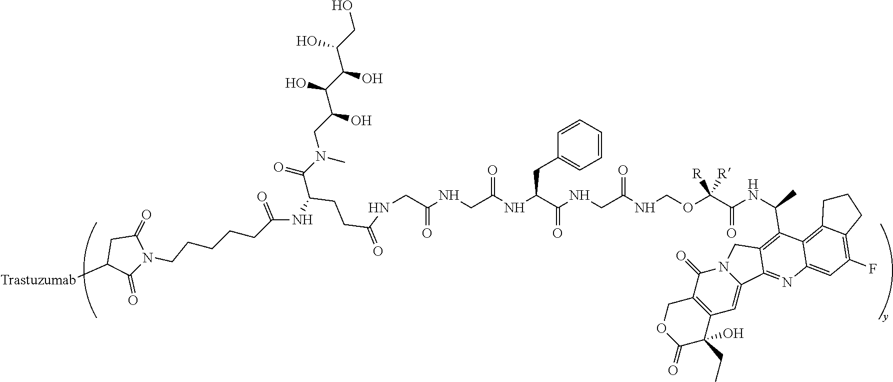

FADC-X₁₄A

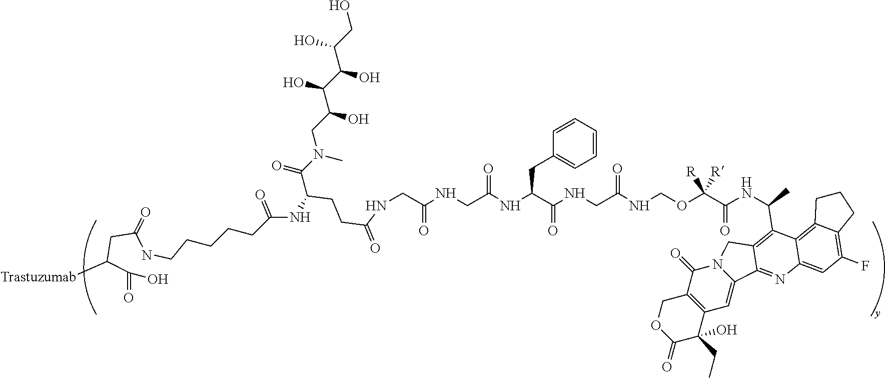

FADC-X₁₄B

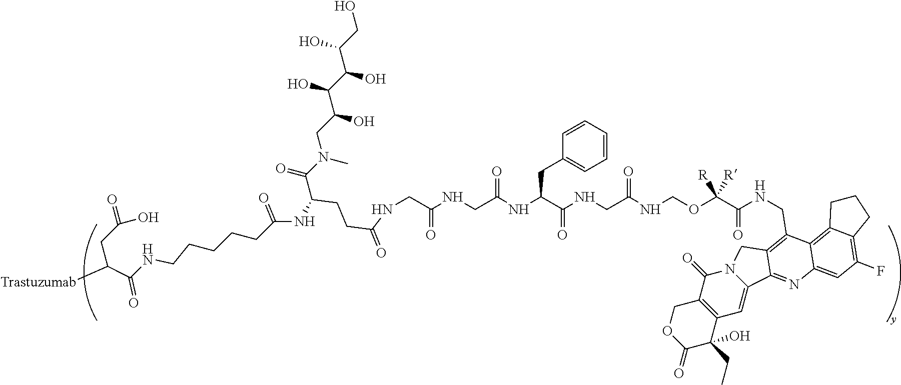

FADC-X₁₄C
wherein, R is selected from —H, R′ is selected from —H;

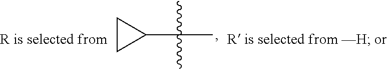 [SMILES: *C1CC1]

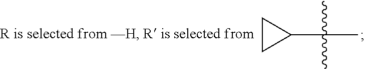 [SMILES: *C1CC1]

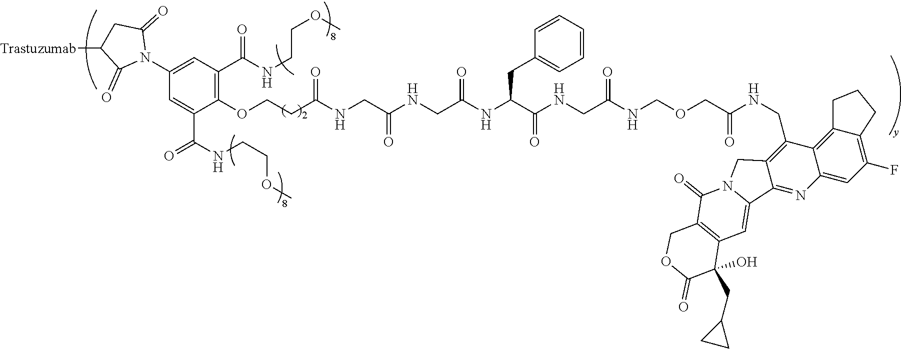

FADC-61A

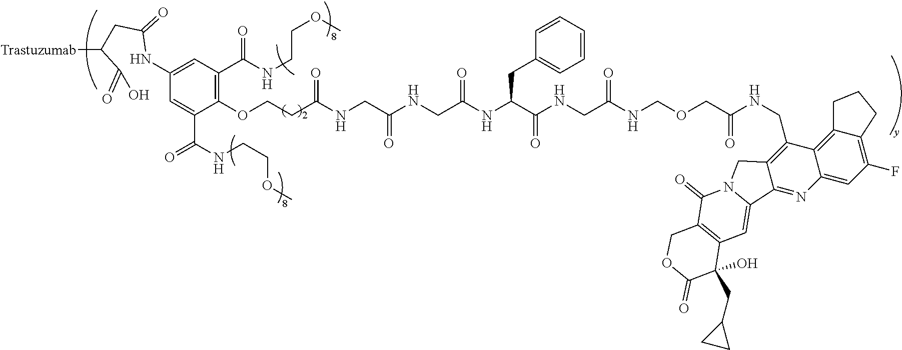

FADC-61B

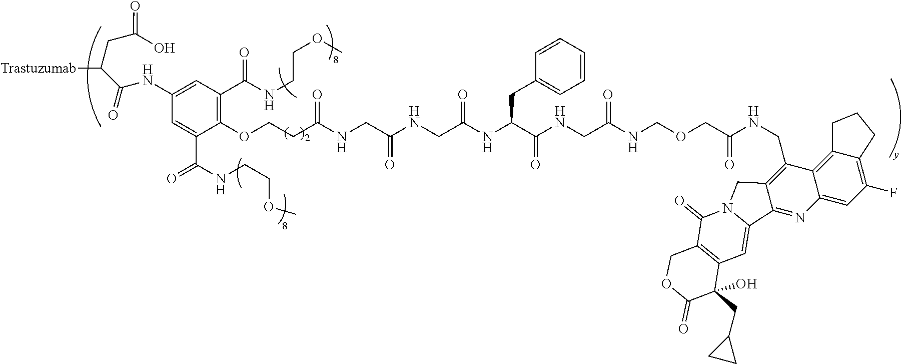

FADC-61C

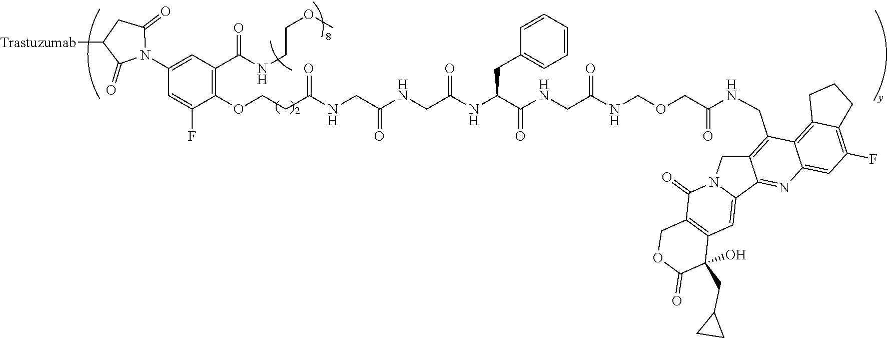

FADC-62A

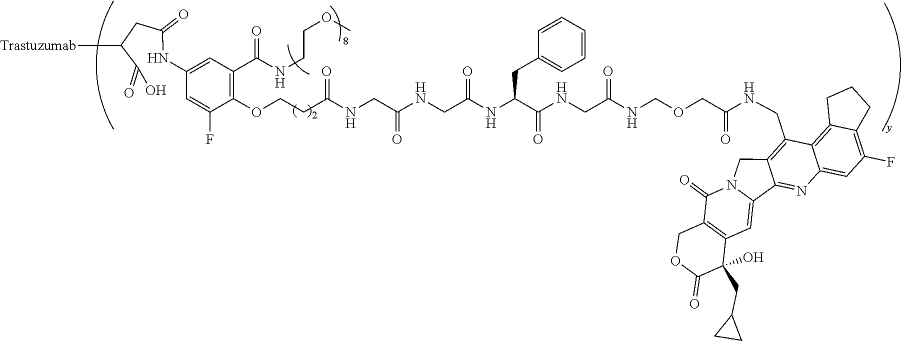

FADC-62B

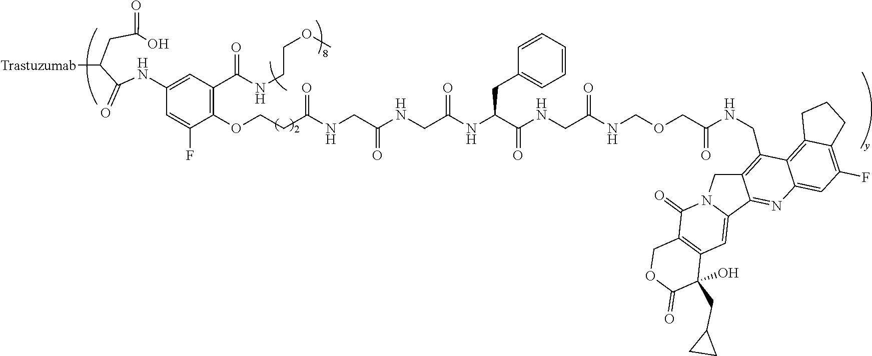

FADC-62C

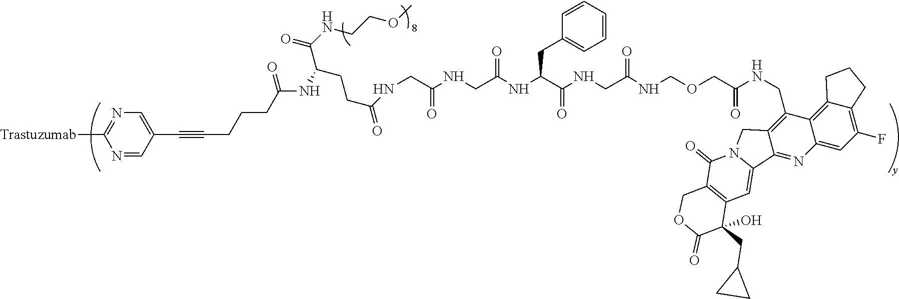

FADC-63

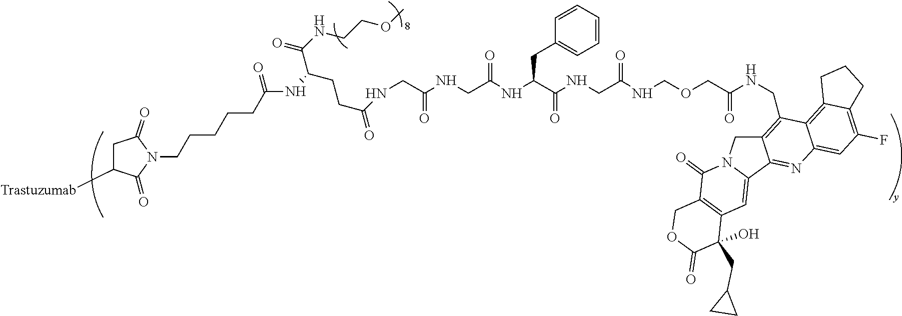

FADC-64A

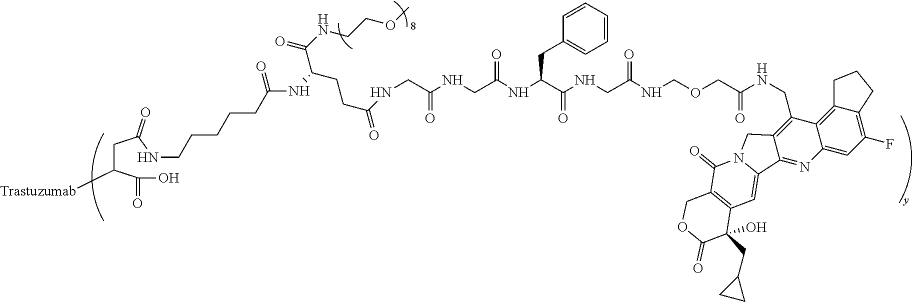

FADC-64B

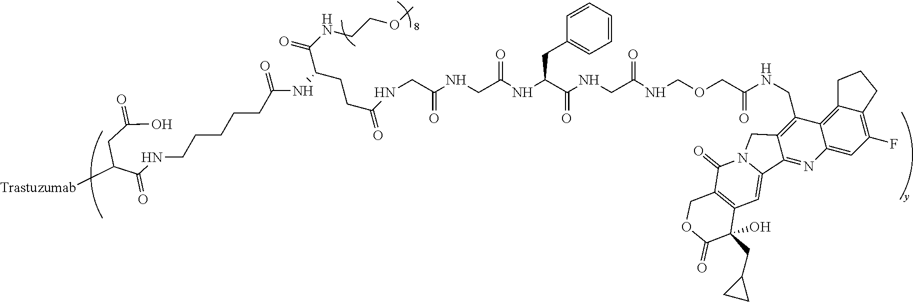

FADC-64C

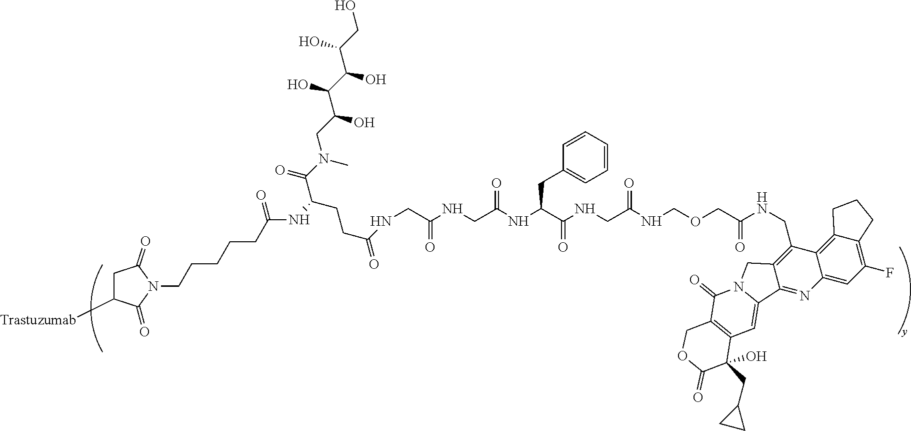

FADC-65A

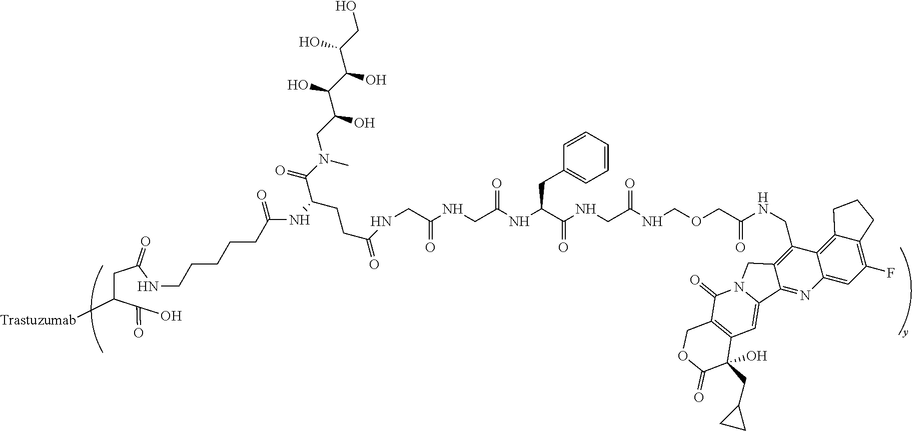

FADC-65B

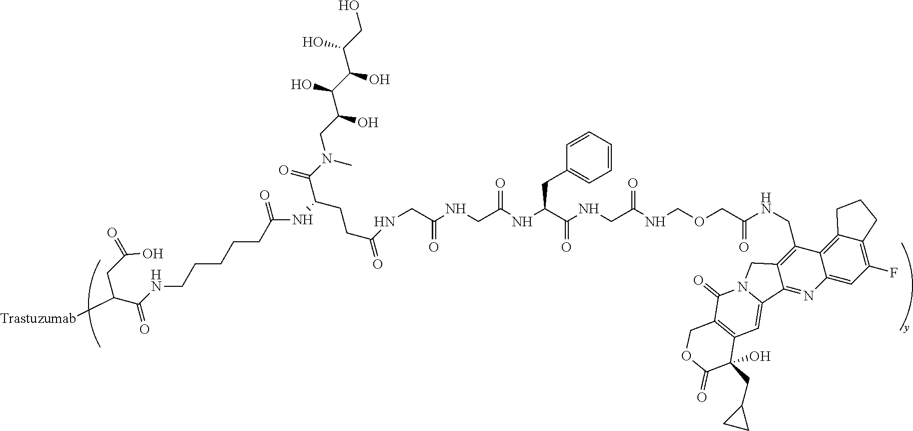

FADC-65C

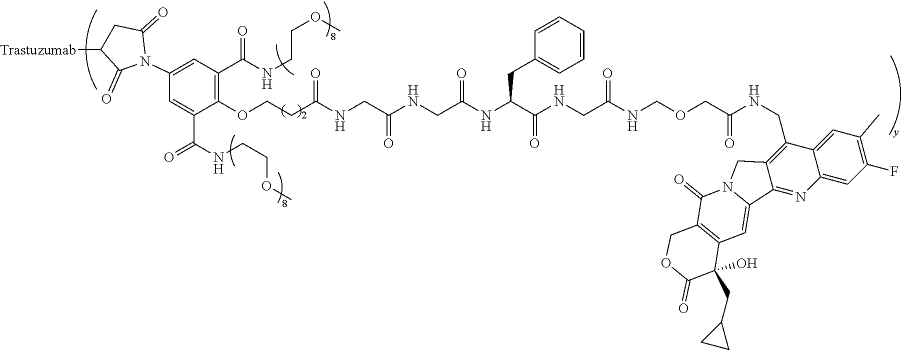

FADC-66A

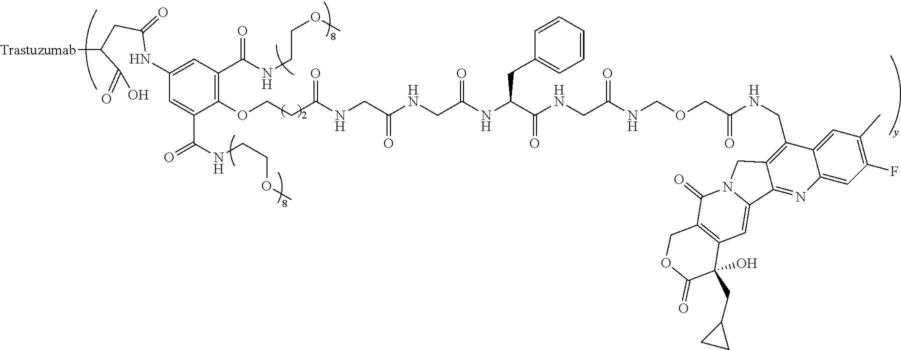

FADC-66B

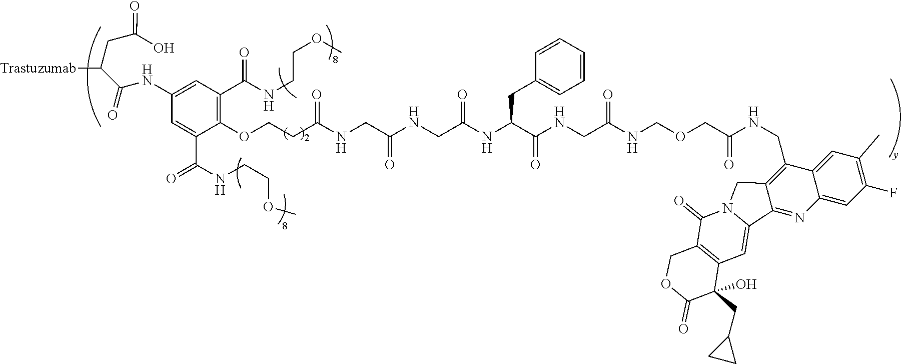

FADC-66C

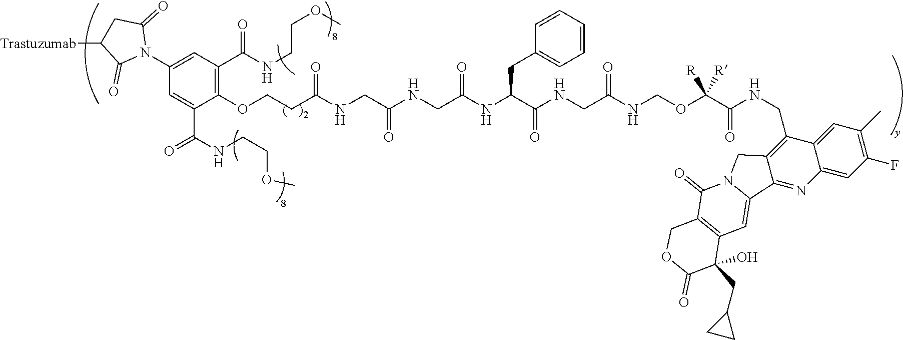

FADC-X₁₅A

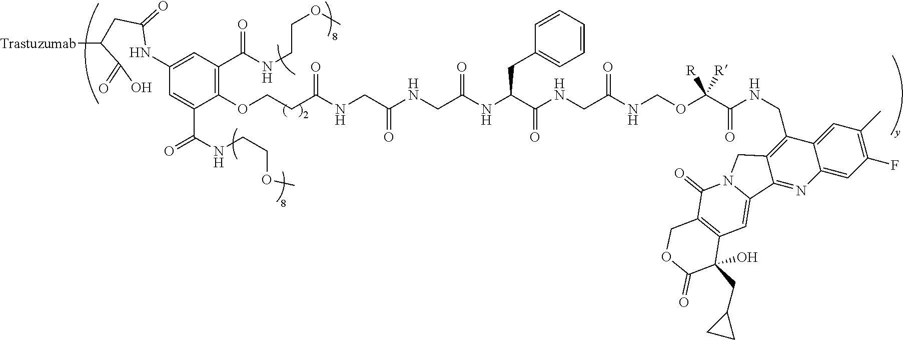

FADC-X₁₅B

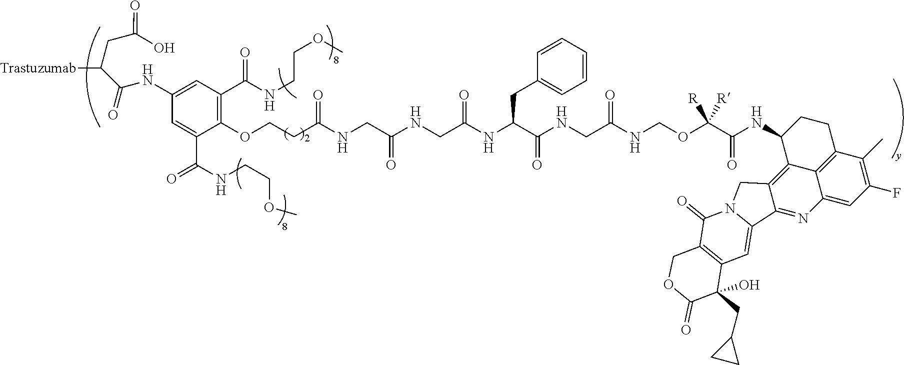

FADC-X₁₅C
wherein, R is selected from —CH₃, R′ is selected from —H;
R is selected from —H, R′ is selected from —CH₃;

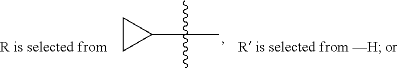 [SMILES: *C1CC1]

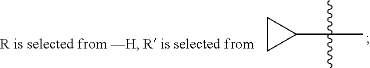 [SMILES: *C1CC1]

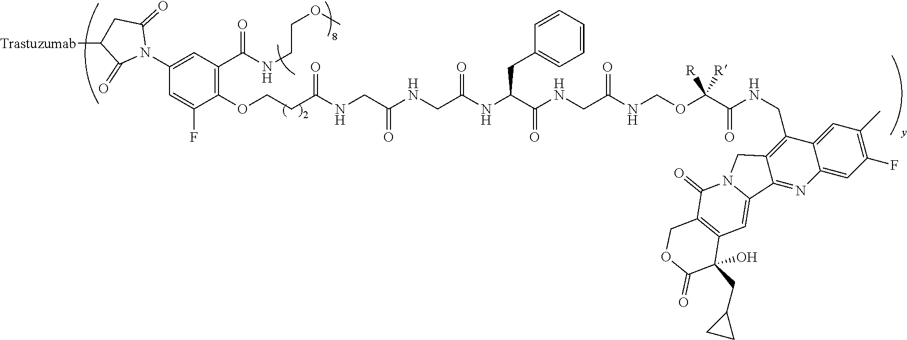

FADC-X₁₆A

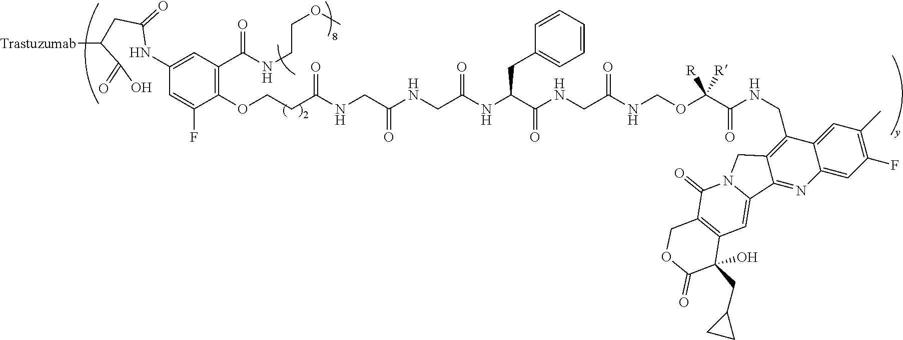

FADC-X₁₆B

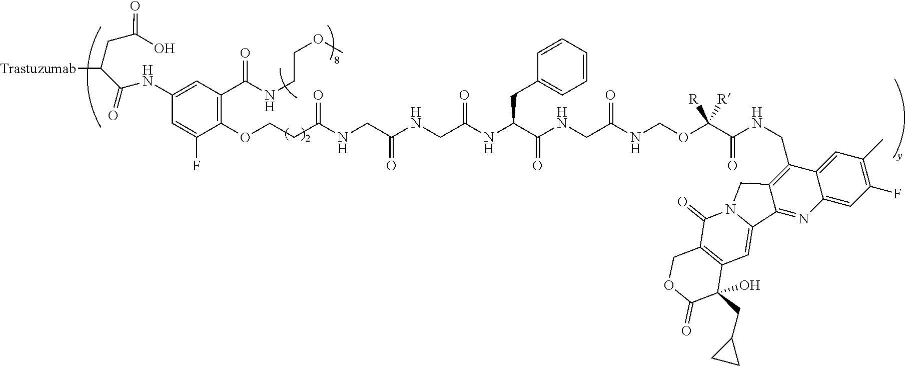

FADC-X₁₆C
wherein, R is selected from —H, R′ is selected from —H;
R is selected from —CH₃, R′ is selected from —H;
R is selected from —H, R′ is selected from —CH₃;

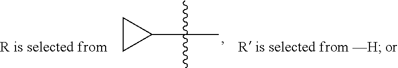 [SMILES: *C1CC1]

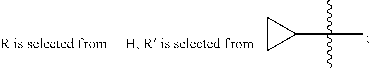 [SMILES: *C1CC1]

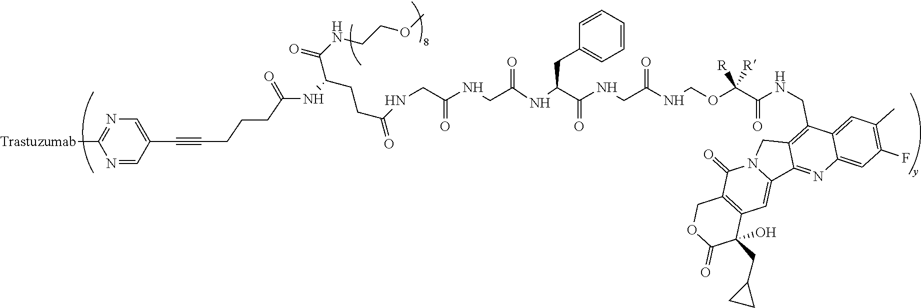

FADC-X₁₇
wherein, R is selected from —H, R′ is selected from —H;
R is selected from —CH₃, R′ is selected from —H;
R is selected from —H, R′ is selected from —CH₃;

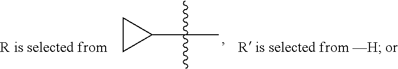 [SMILES: *C1CC1]

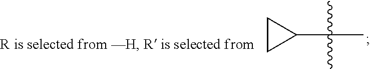 [SMILES: *C1CC1]

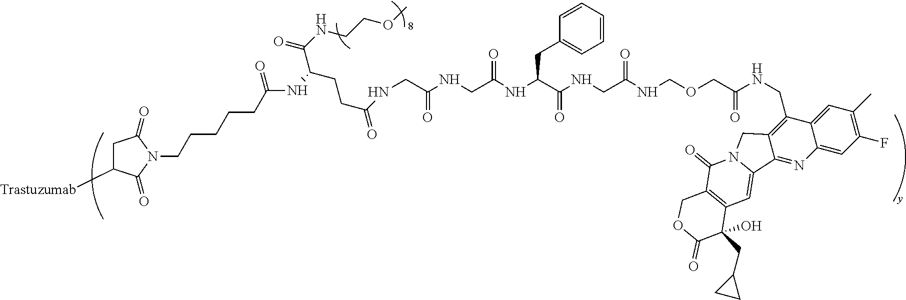

FADC-81A

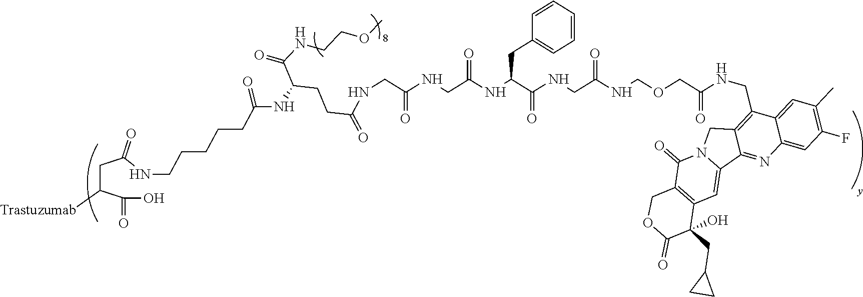

FADC-81B

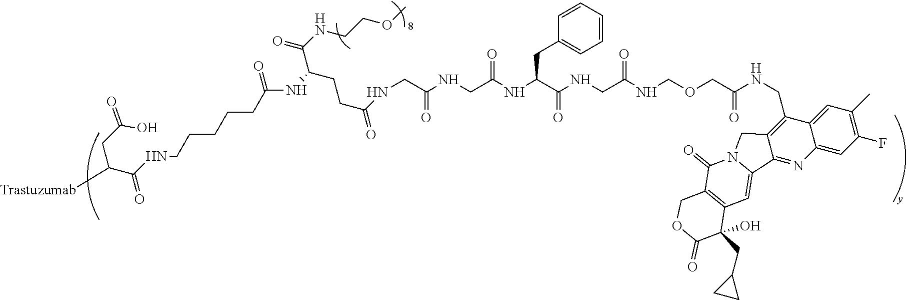

FADC-81C

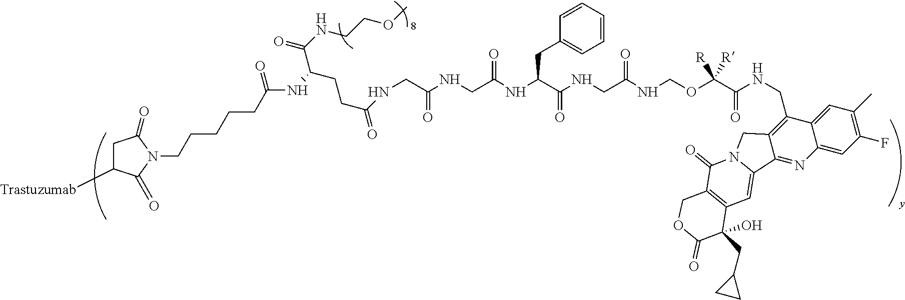

FADC-X₁₈A

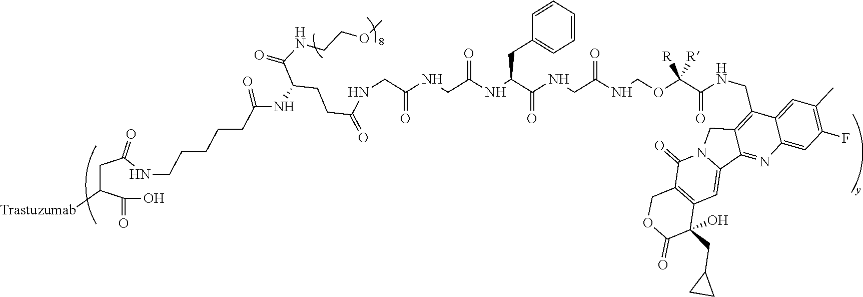

FADC-X₁₈B

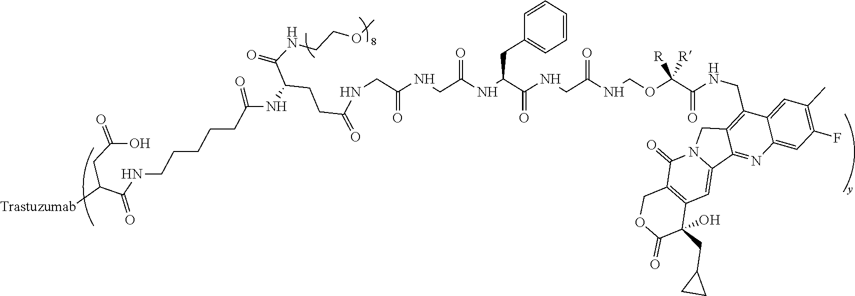

FADC-X₁₈C
wherein, R is selected from —CH₃, R′ is selected from —H;
R is selected from —H, R′ is selected from —CH₃;

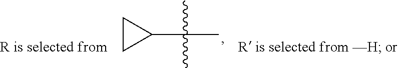 [SMILES: *C1CC1]

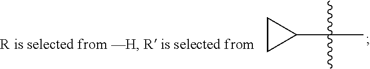 [SMILES: *C1CC1]

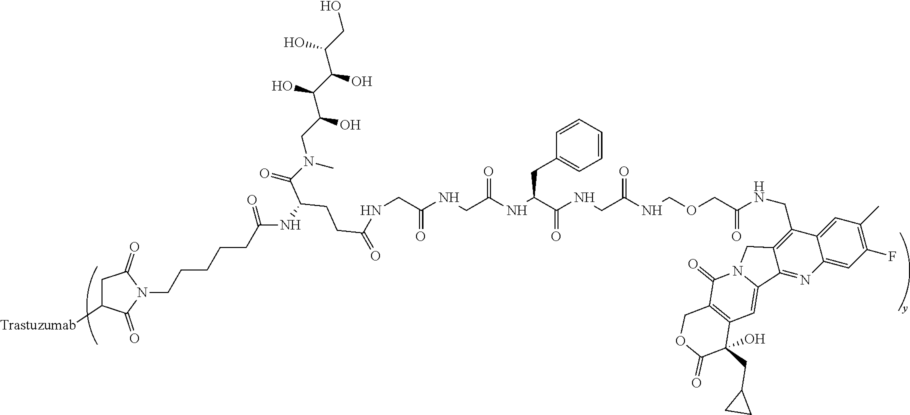

FADC-86A

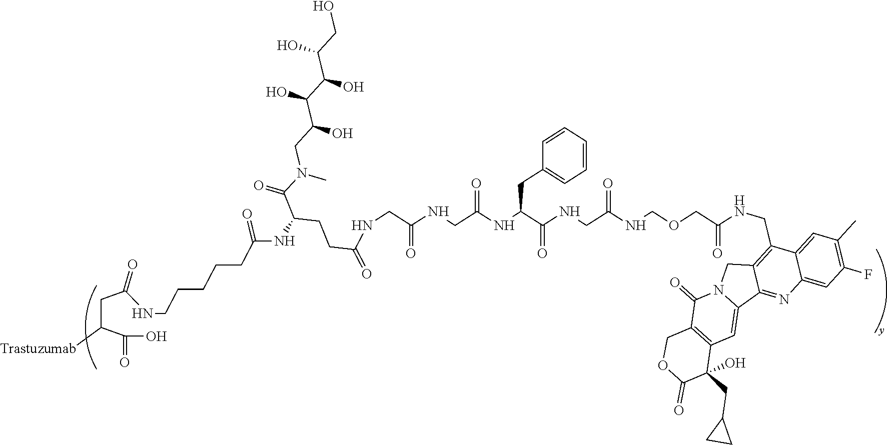

FADC-86B

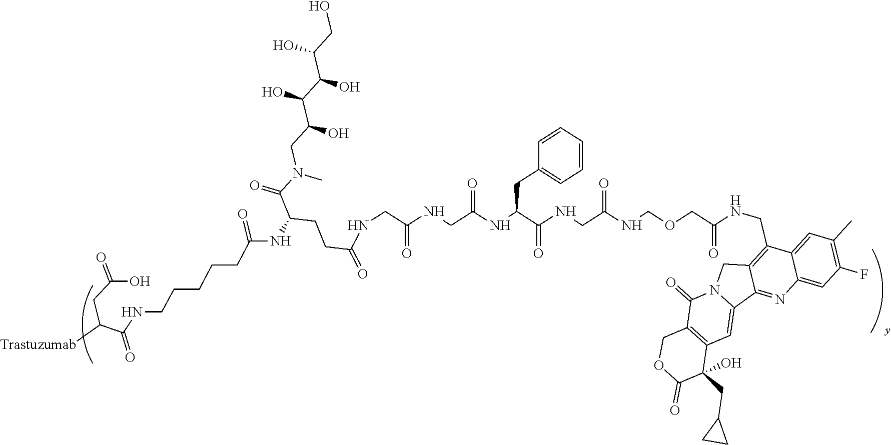

FADC-86C

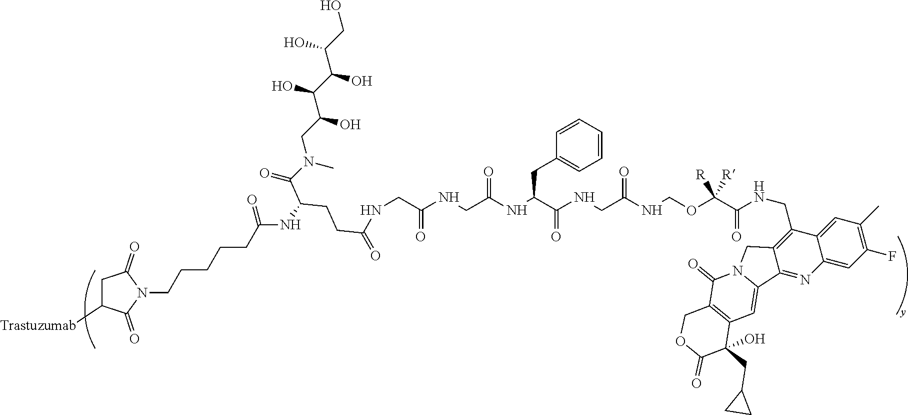

FADC-X₁₉A

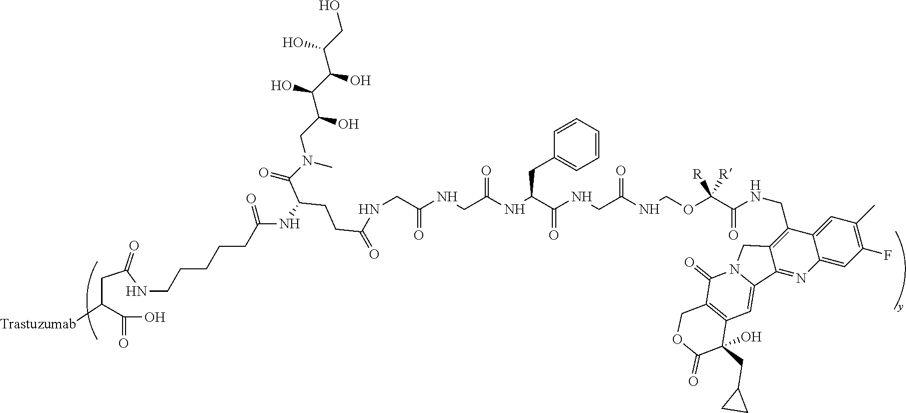

FADC-X₁₉B

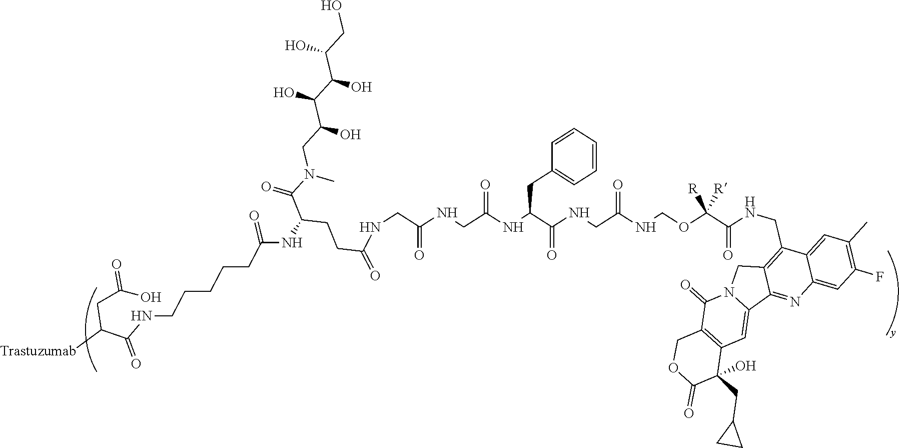

FADC-X₁₉C
wherein, R is selected from —CH₃, R′ is selected from —H;
R is selected from —H, R′ is selected from —CH₃;

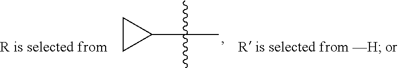 [SMILES: *C1CC1]

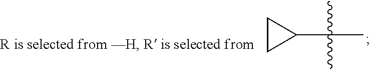 [SMILES: *C1CC1]

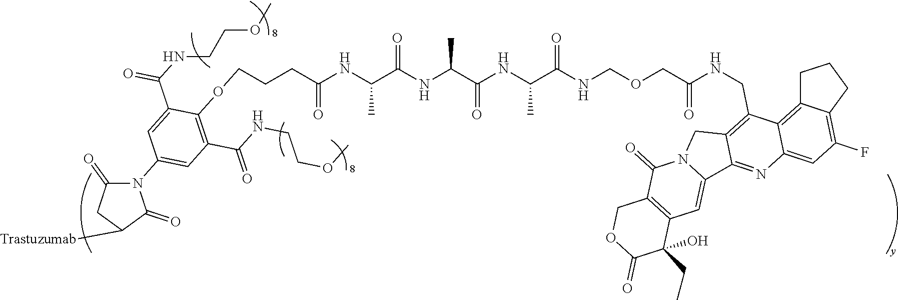

FADC-91A

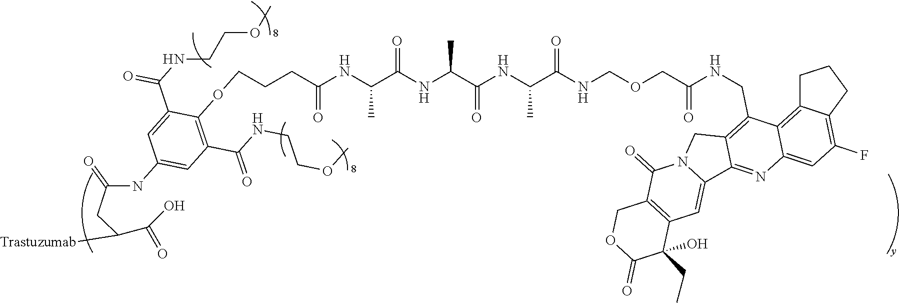

FADC-91B

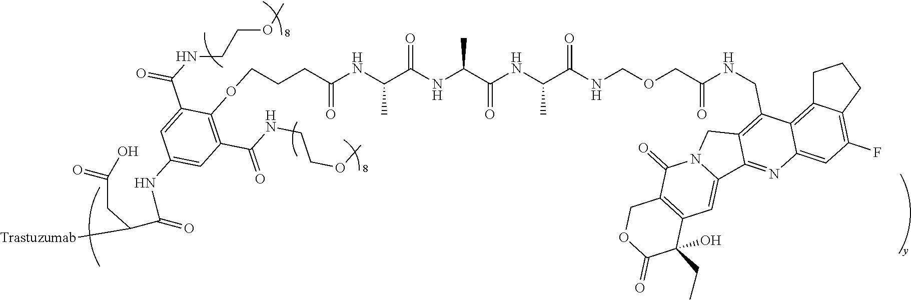

FADC-91C

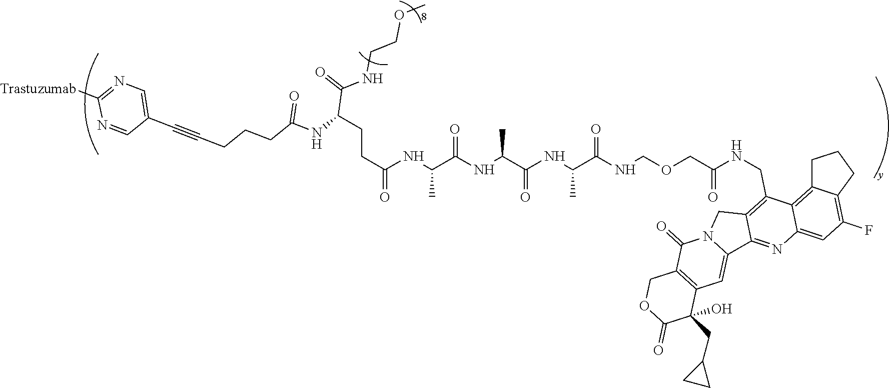

FADC-92

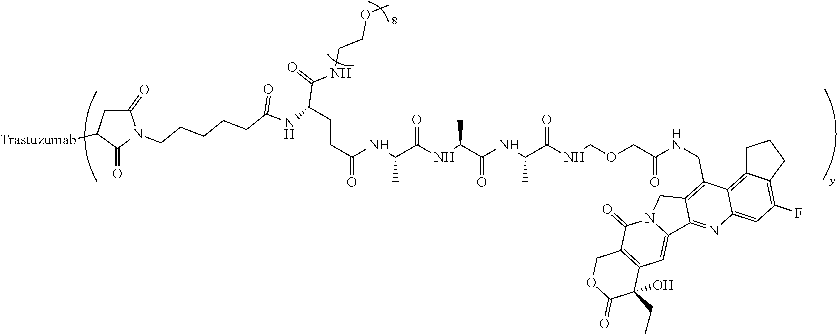

FADC-93A

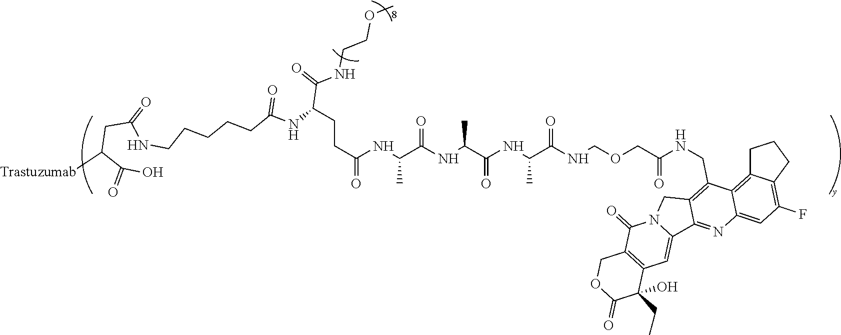

FADC-93B

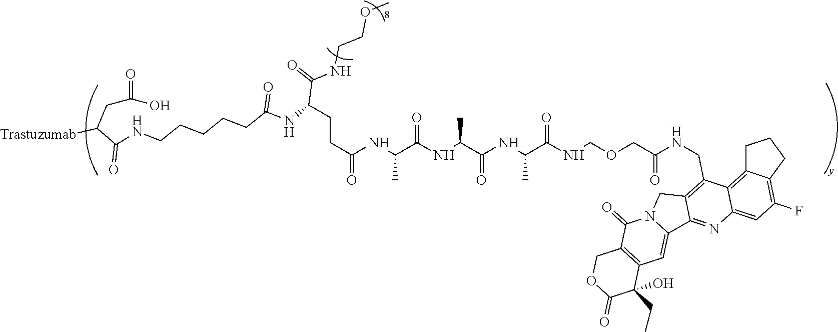

FADC-93C

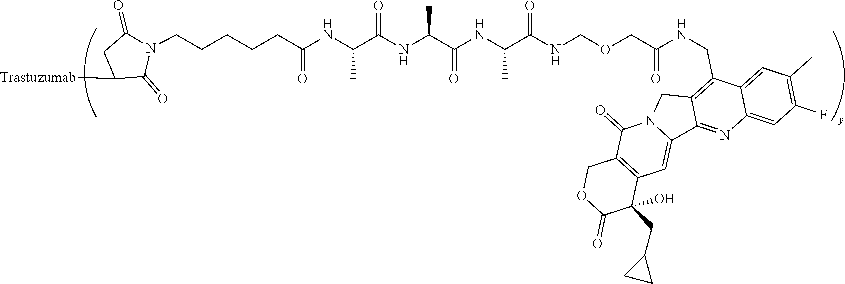

FADC-94A

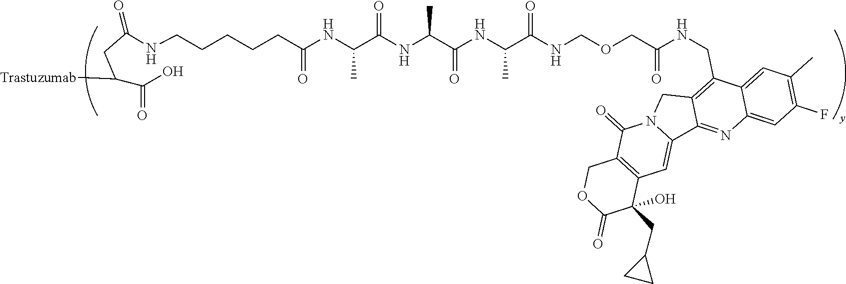

FADC-94B

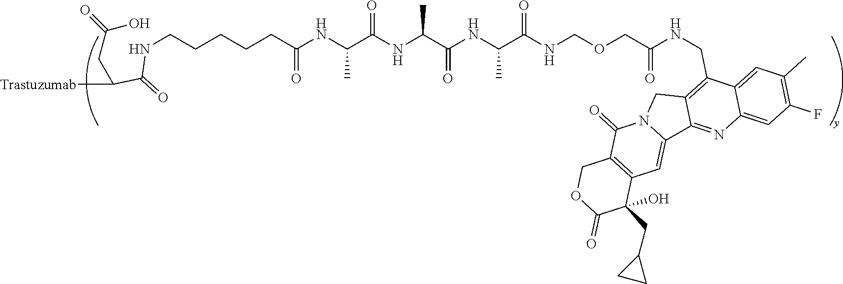

FADC-94C

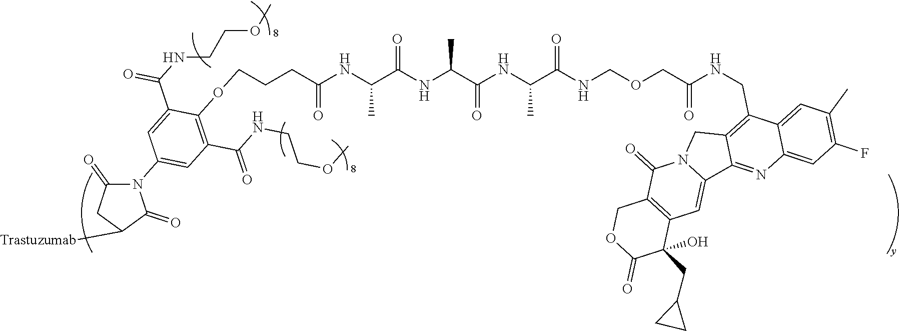

FADC-95A

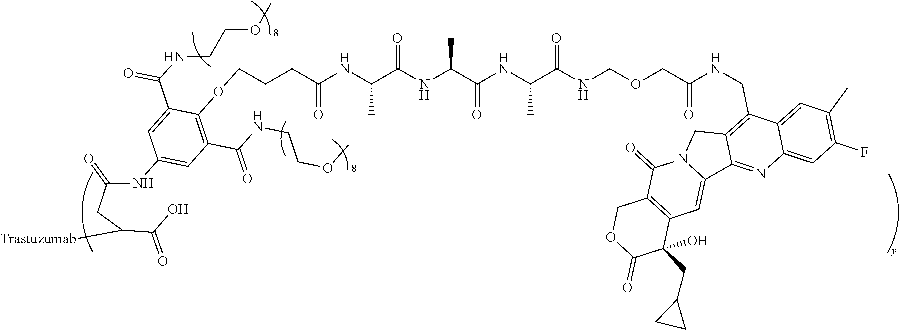

FADC-95B

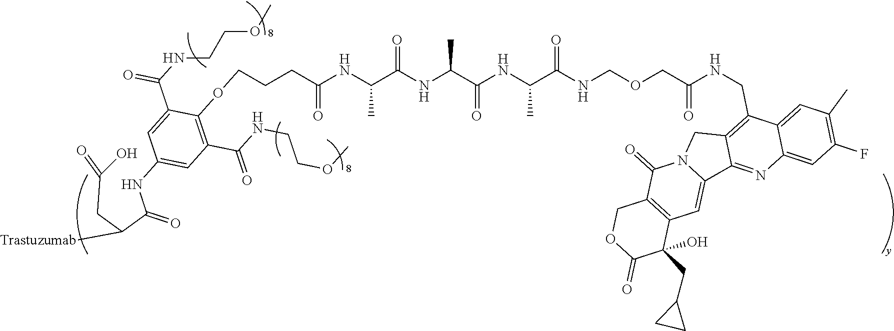

FADC-95C

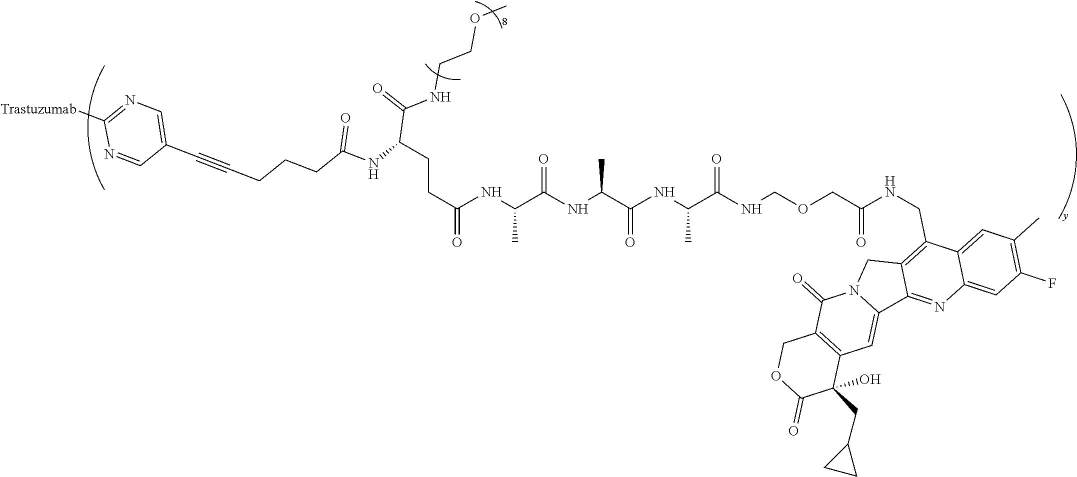

FADC-96

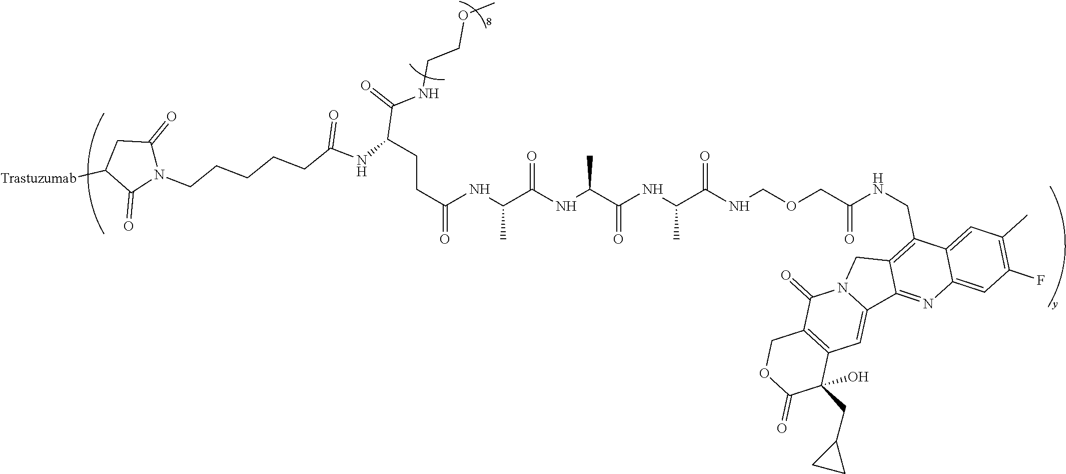

FADC-97A

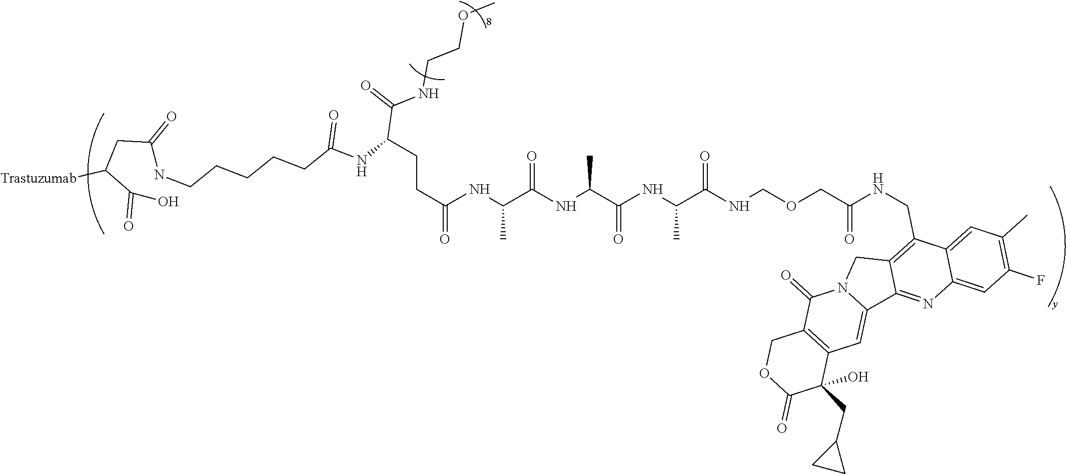

FADC-97B

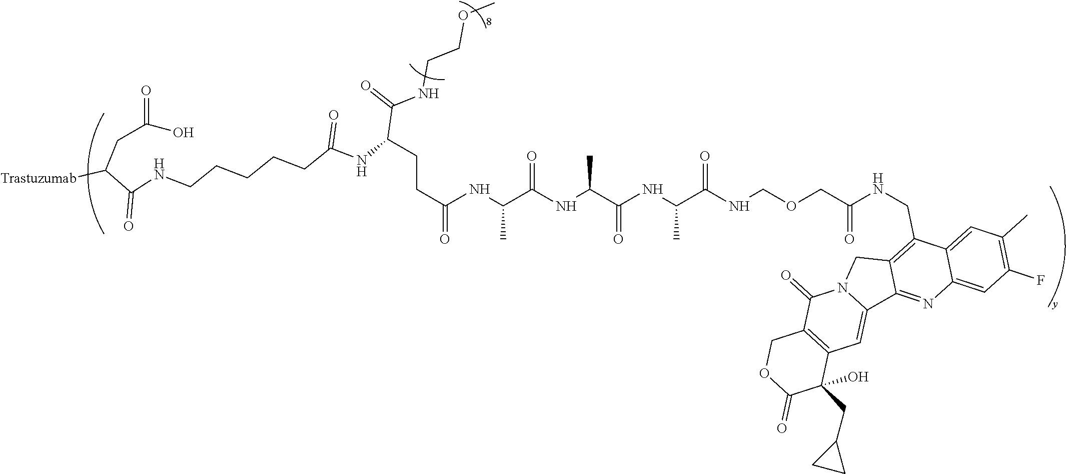

FADC-97C

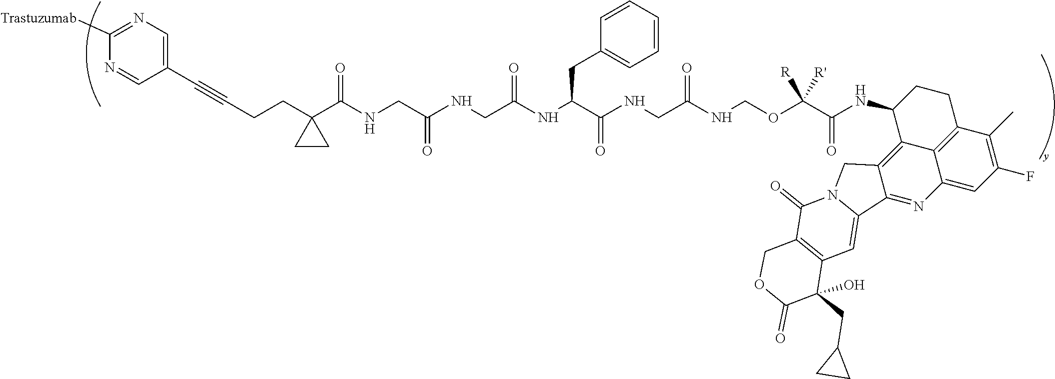

FADC-X₂₀
wherein, R = —H, R′ = —H;
R = —CH₃, R′ = —H;
R = —H, R′ = —CH₃;

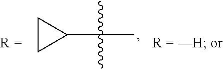 [SMILES: *C1CC1]

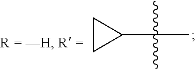 [SMILES: *C1CC1]

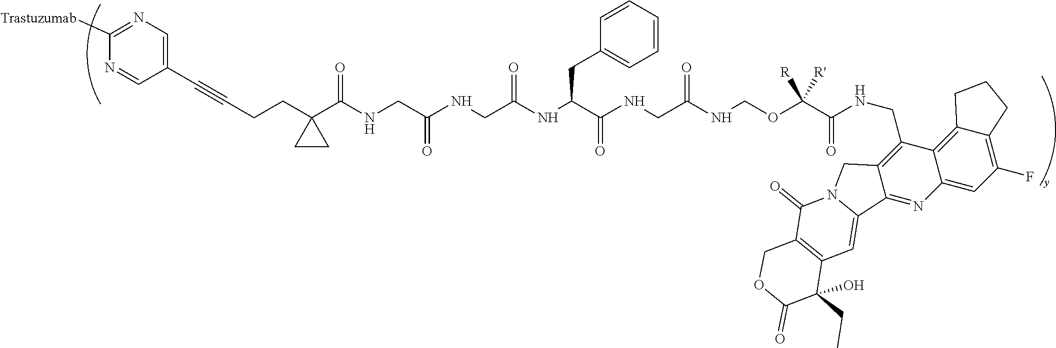

FADC-X₂₁
wherein, R = —H, R′ = —H;
R = —CH₃, R′ = —H; or
R = —H, R′ = —CH₃;

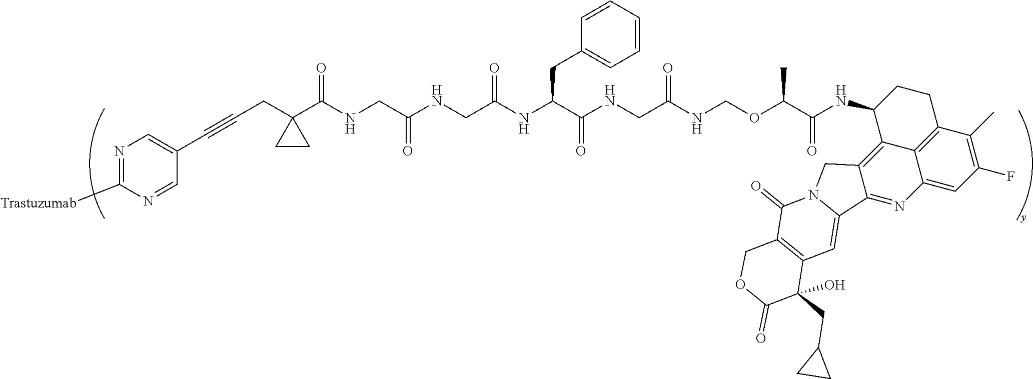

FADC-104

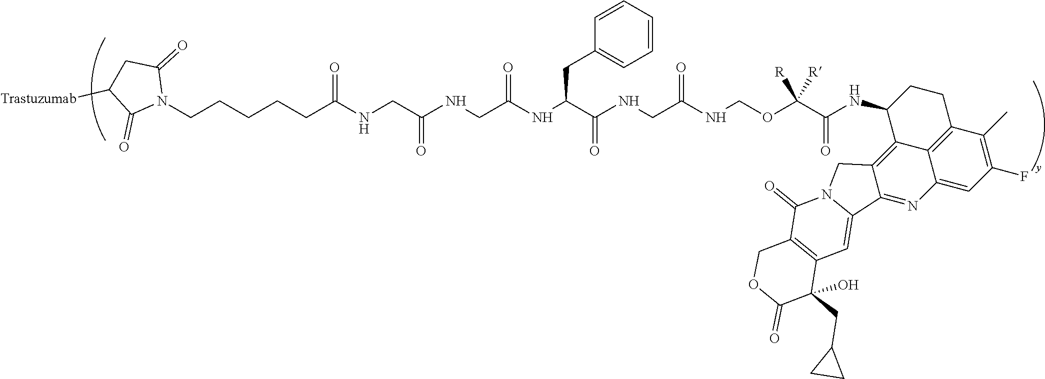

FADC-X₂₂A

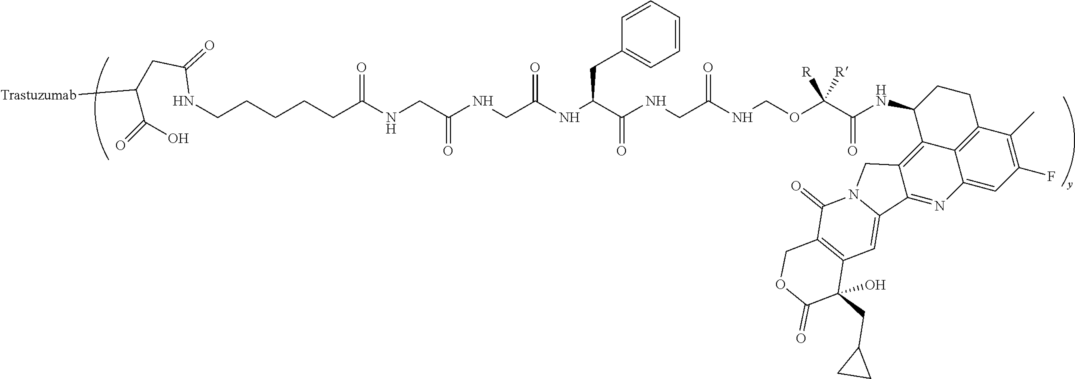

FADC-X₂₂B

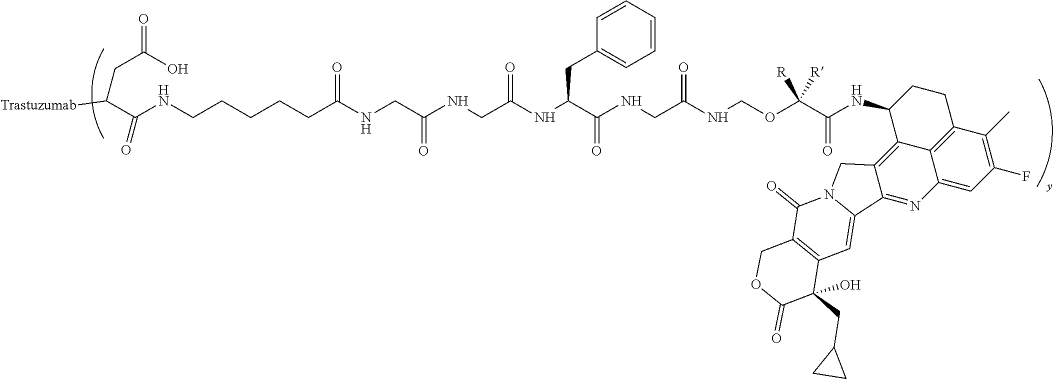

FADC-X₂₂C
wherein, R = —H, R′ = —CH₃; or
R = —CH₃, R′ = —H;

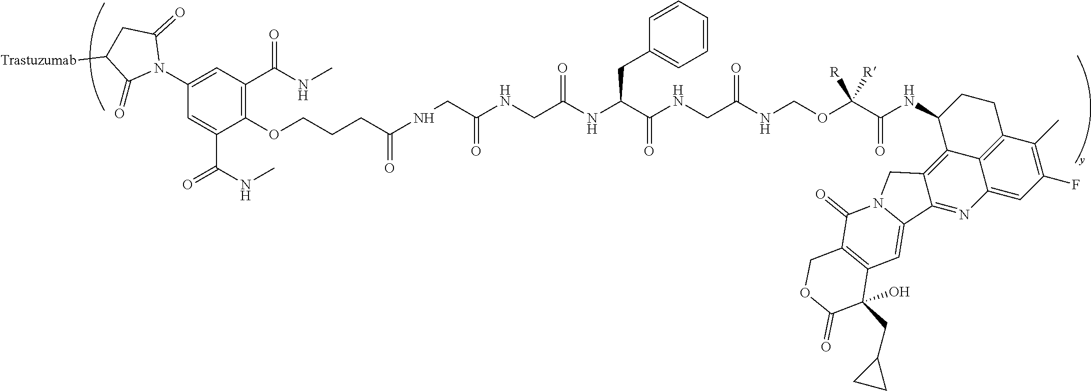

FADC-X₂₃A

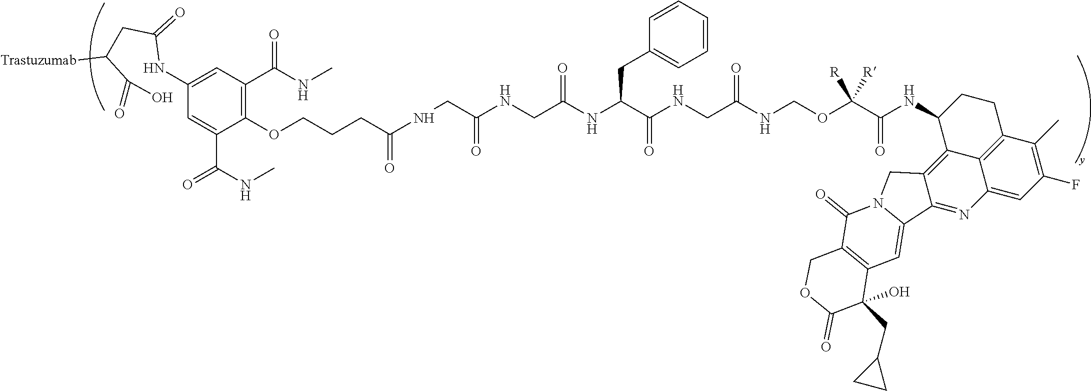

FADC-X₂₃B

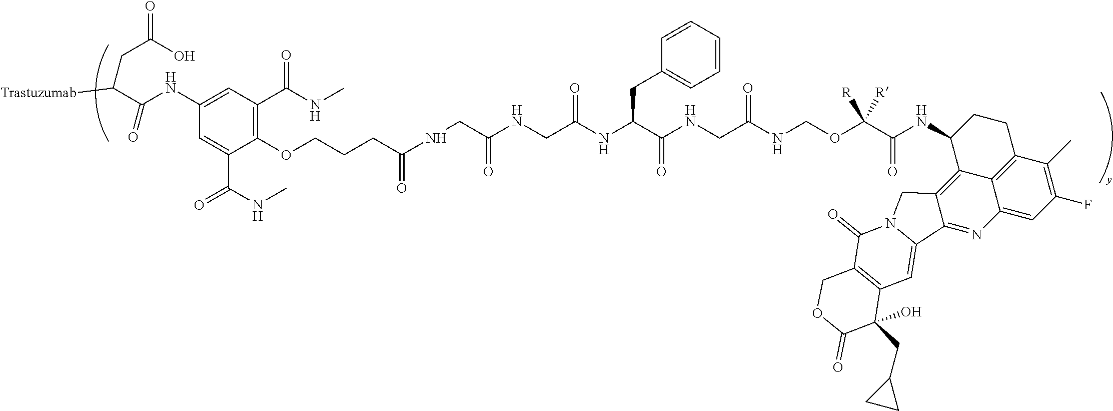

FADC-X₂₃C
wherein, R = —H, R′ = —H;
R = —CH₃, R′ = —H;

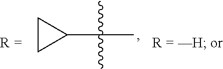 [SMILES: *C1CC1]

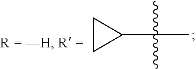 [SMILES: *C1CC1]

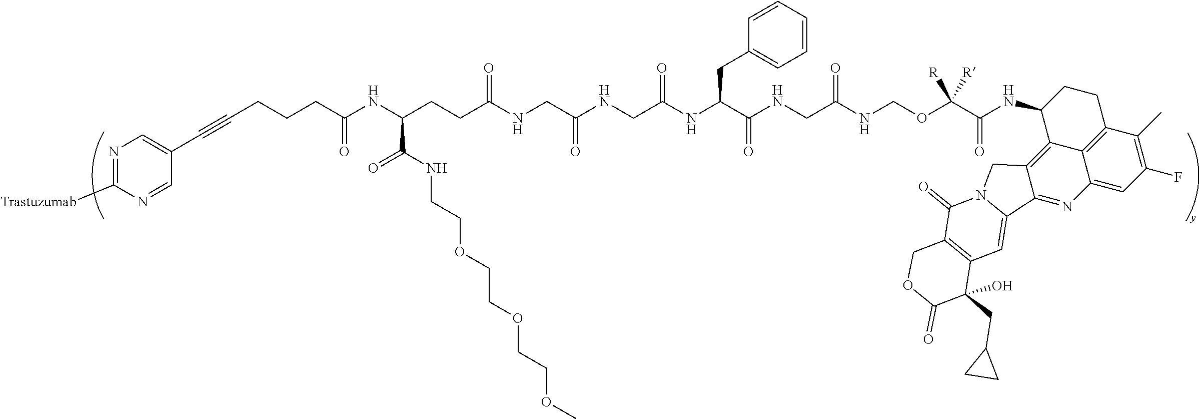

FADC-X₂₄
wherein, R = —H, R′ = —H;
R = —CH₃, R′ = —H;
R = —H, R′ = —CH₃;

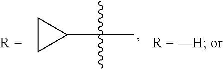 [SMILES: *C1CC1]

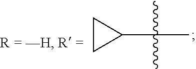 [SMILES: *C1CC1]

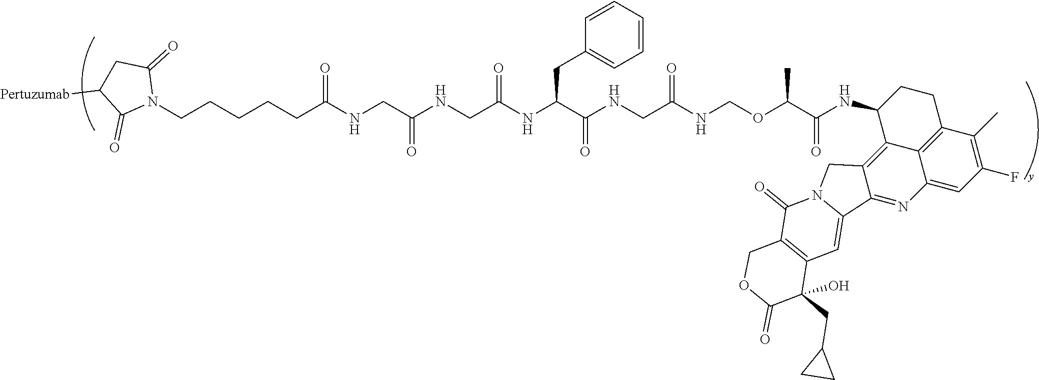

FADC-118A

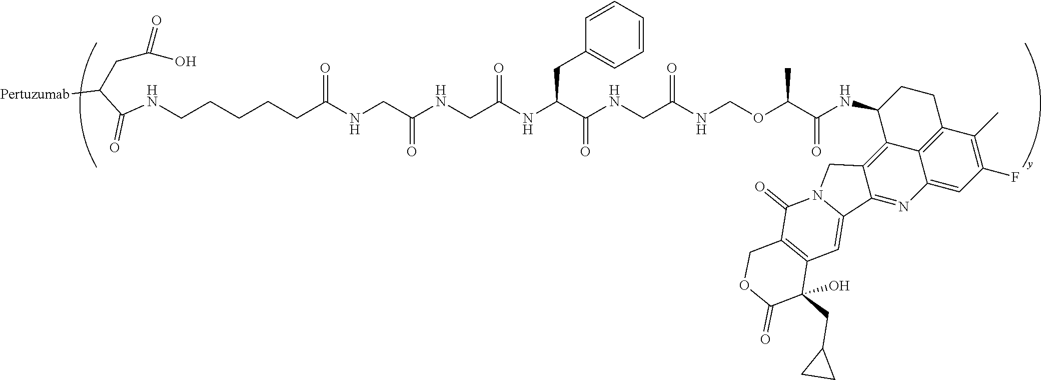

FADC-118B

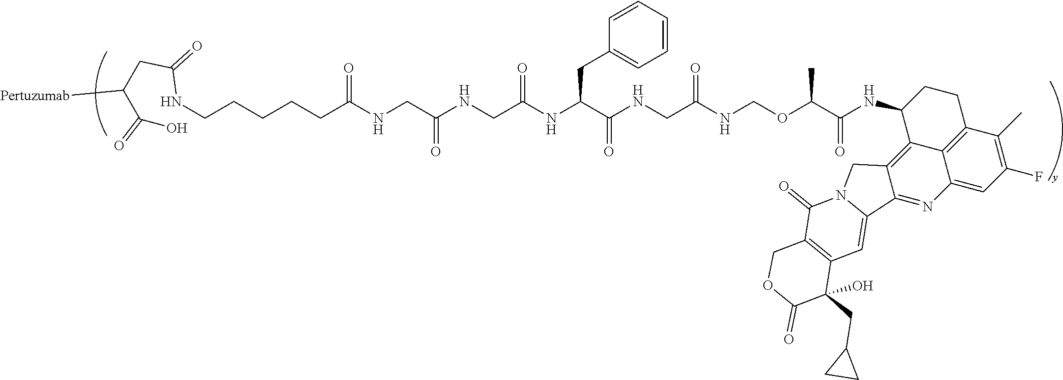

FADC-118C

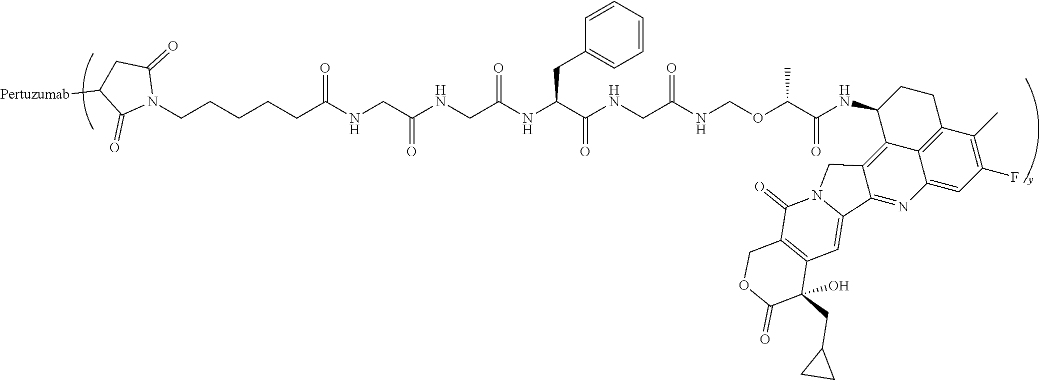

FADC-119A

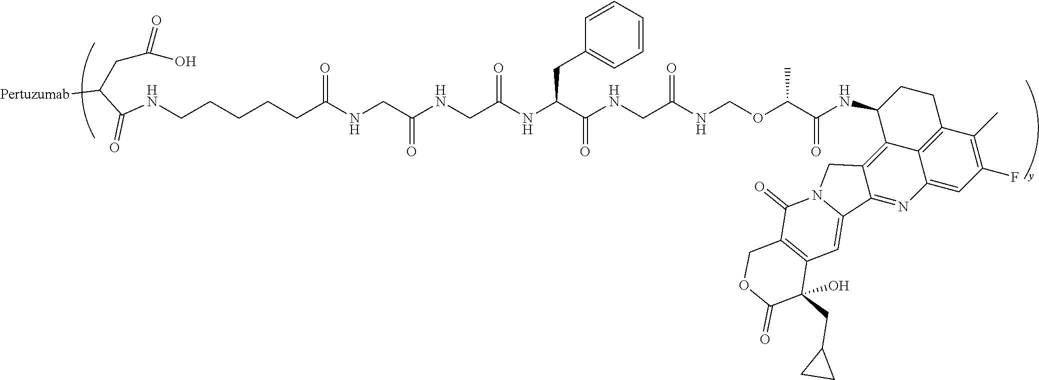

FADC-119B

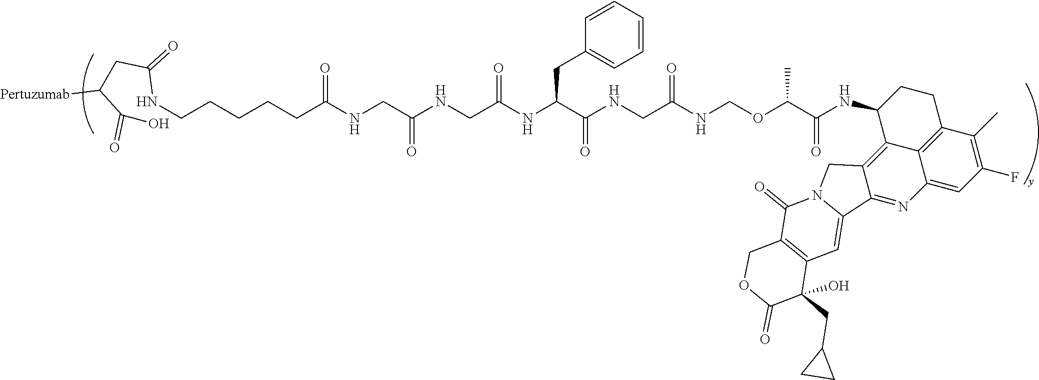

FADC-119C

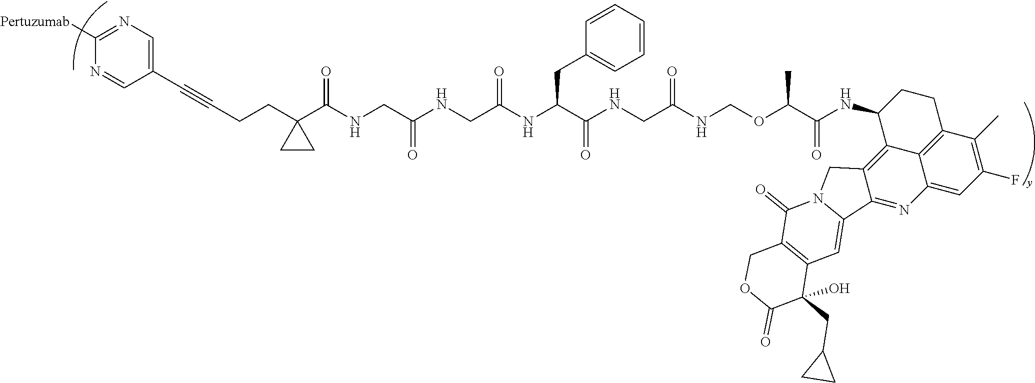

FADC-120

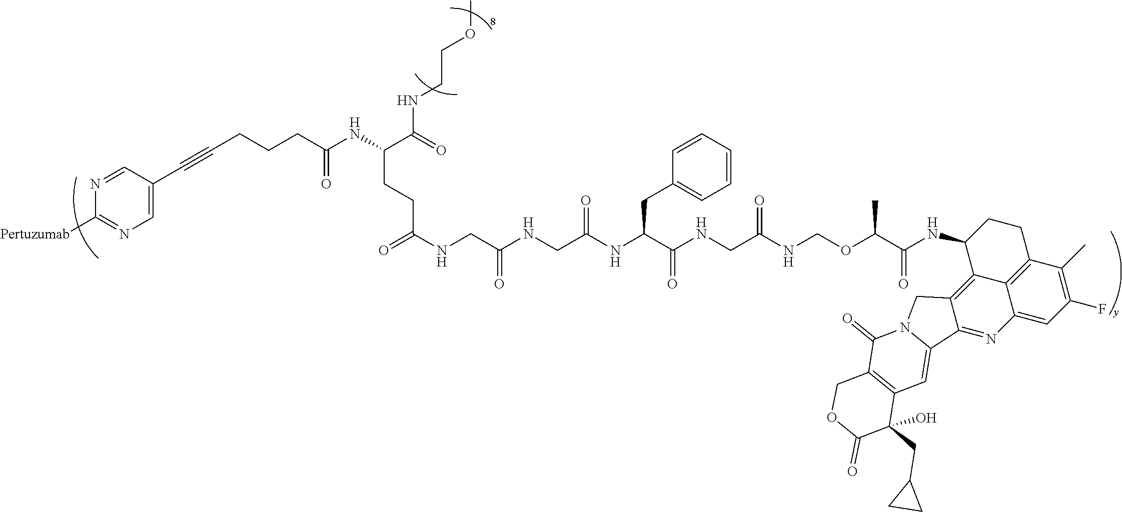

FADC-121

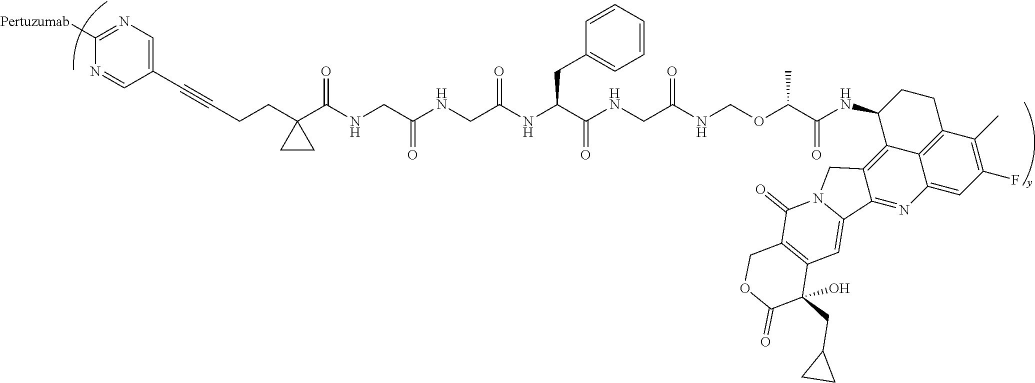

FADC-122

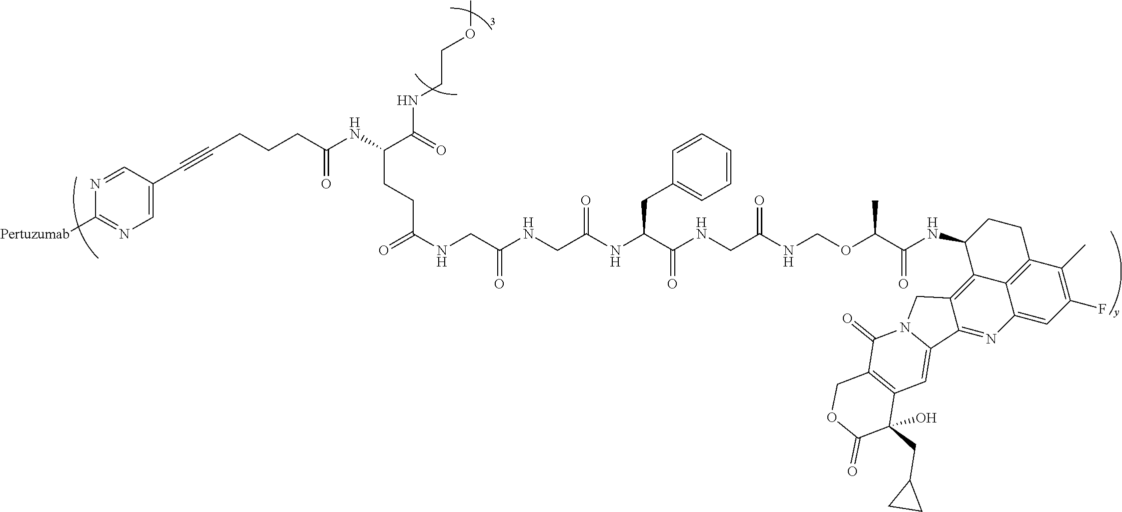

FADC-123

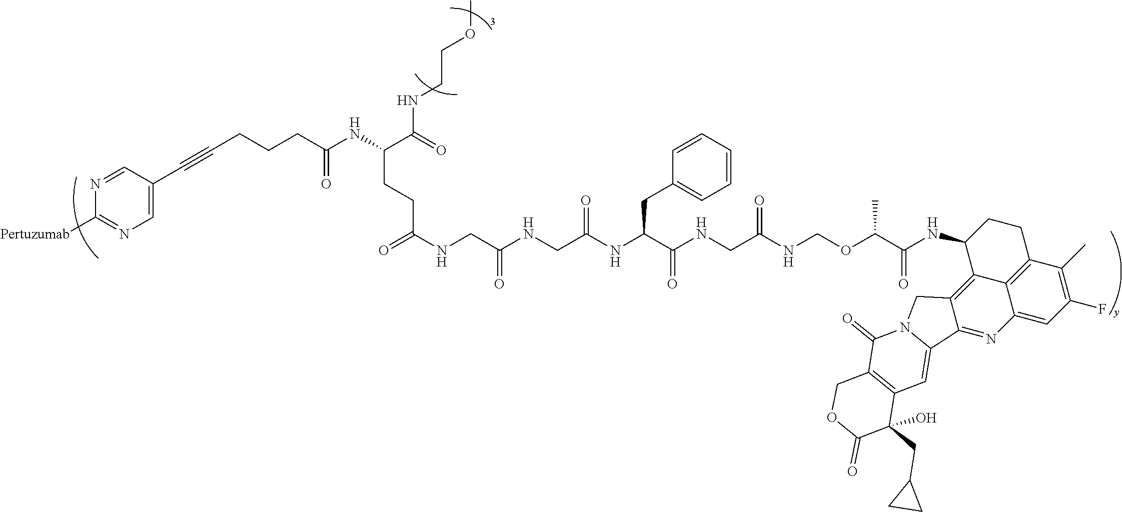

FADC-124

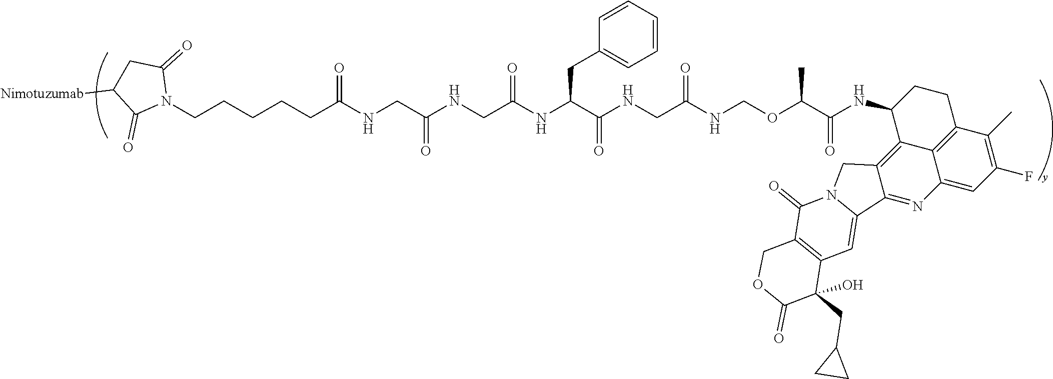

FADC-125A

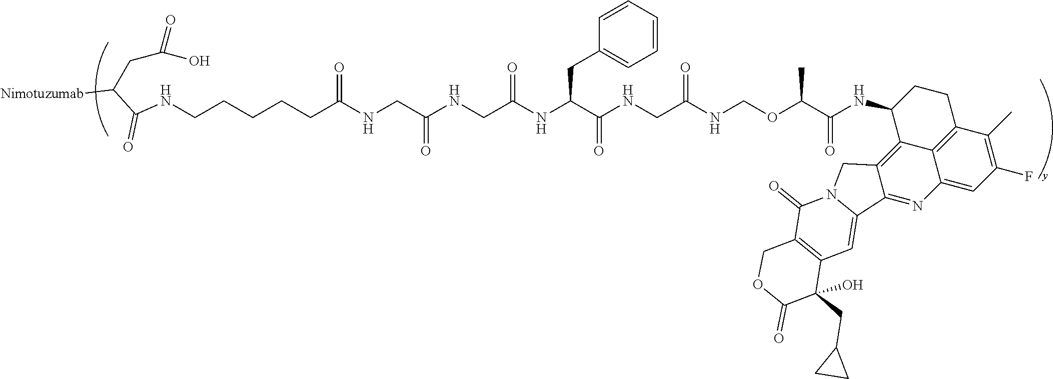

FADC-125B

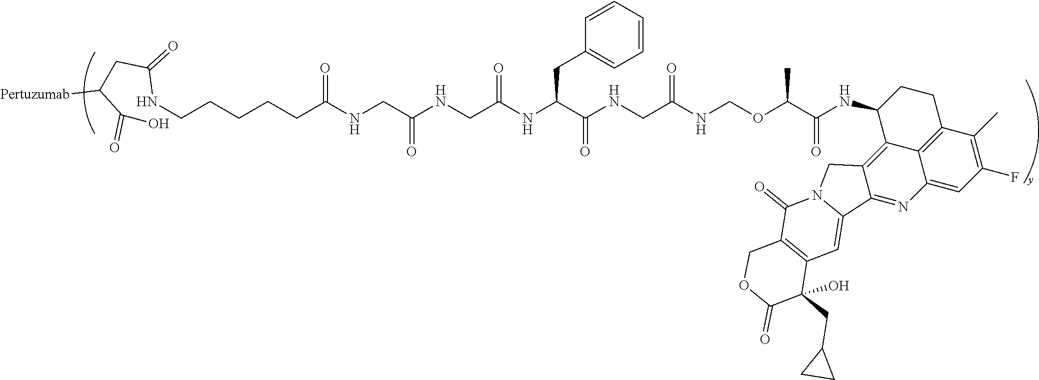

FADC-125C

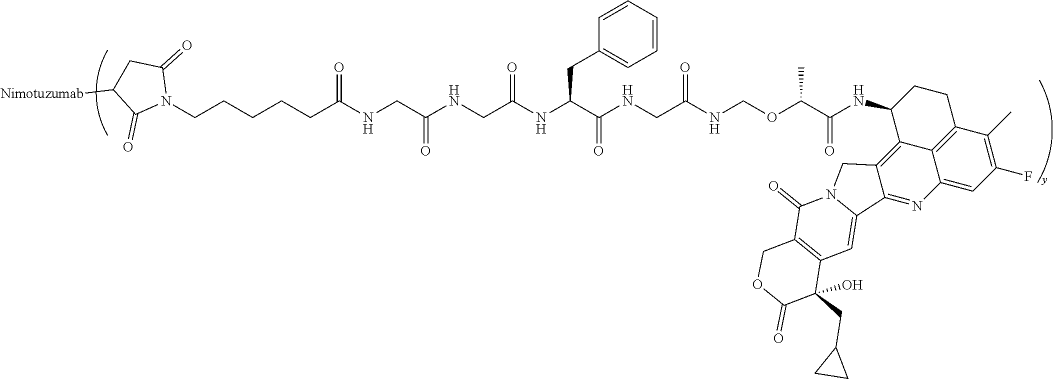

FADC-126A

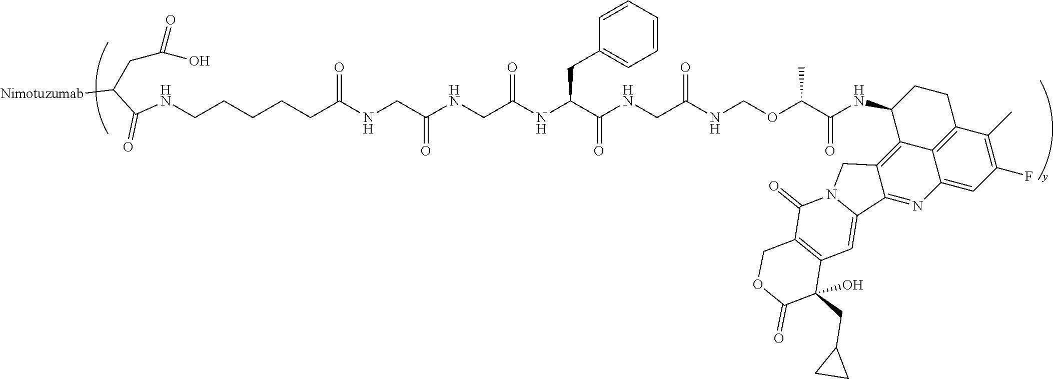

FADC-126B

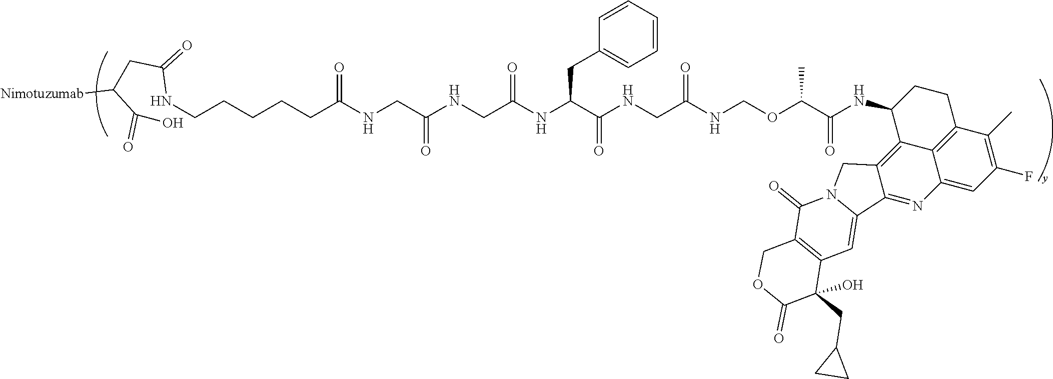

FADC-126C

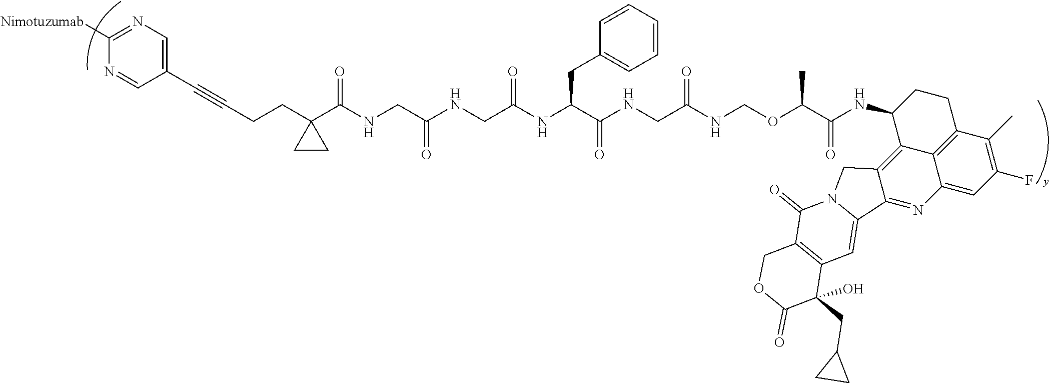

FADC-127

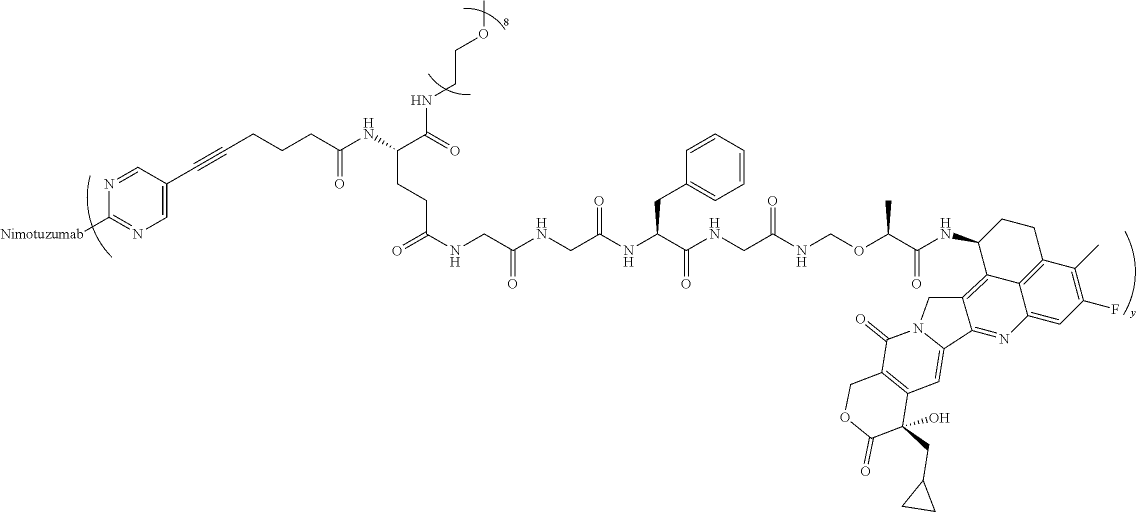

FADC-128

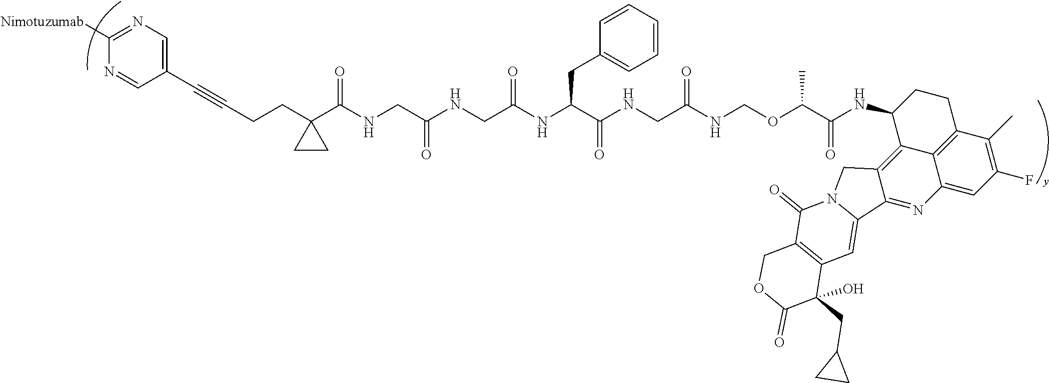

FADC-129

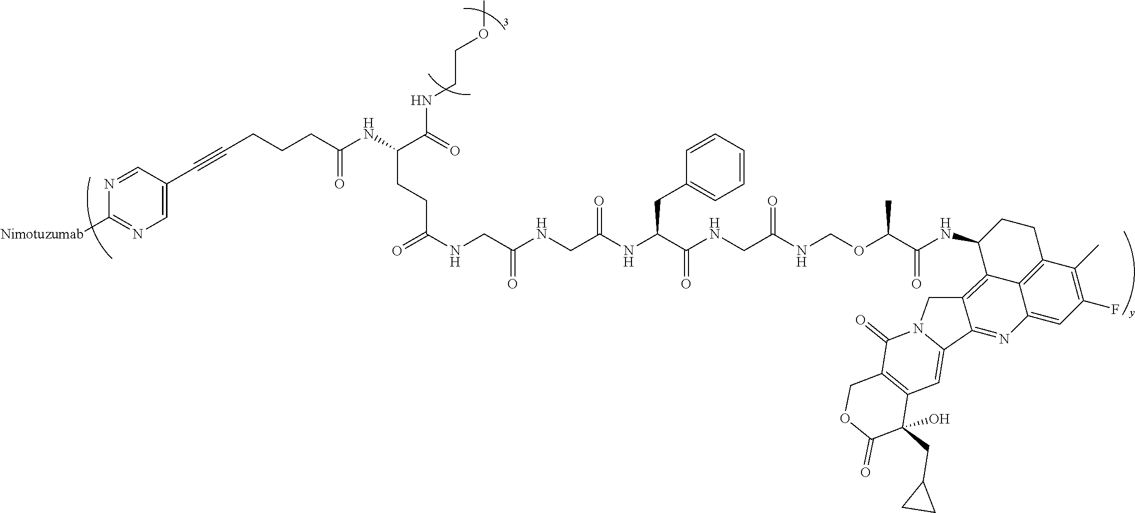

FADC-130

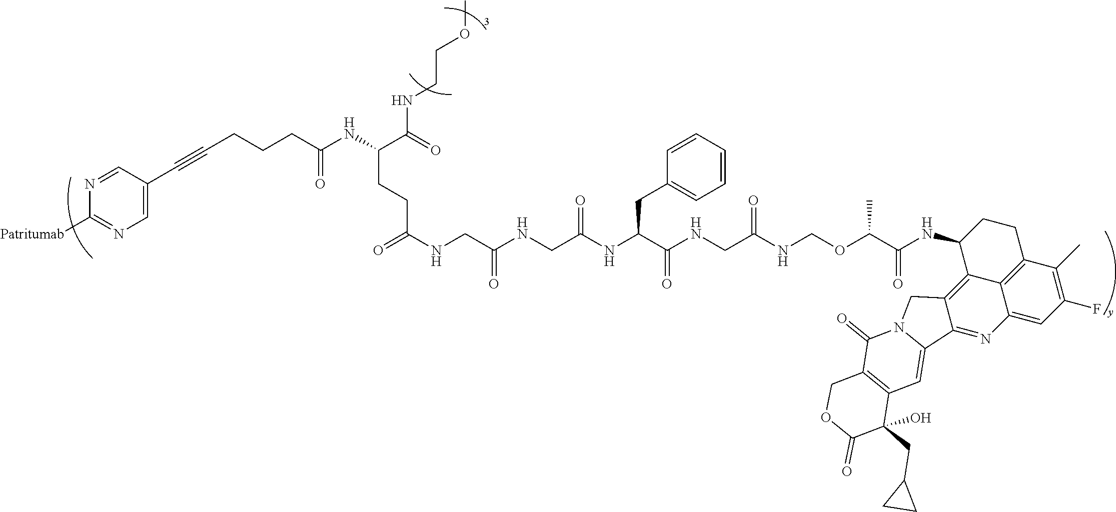

FADC-131

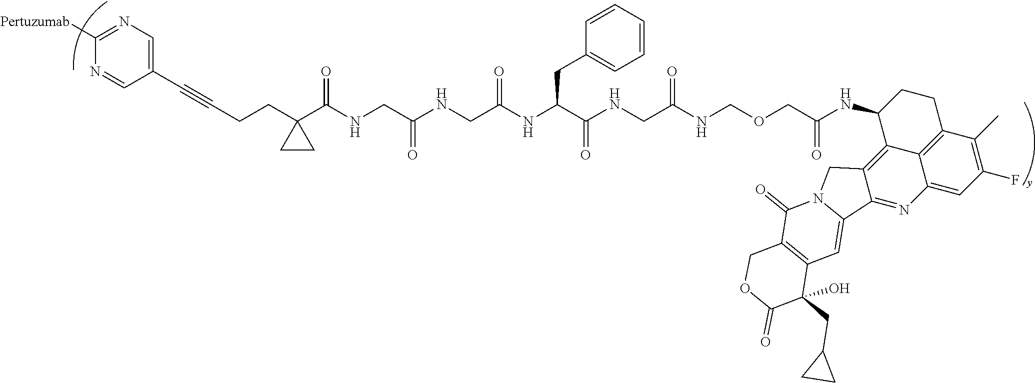

FADC-132

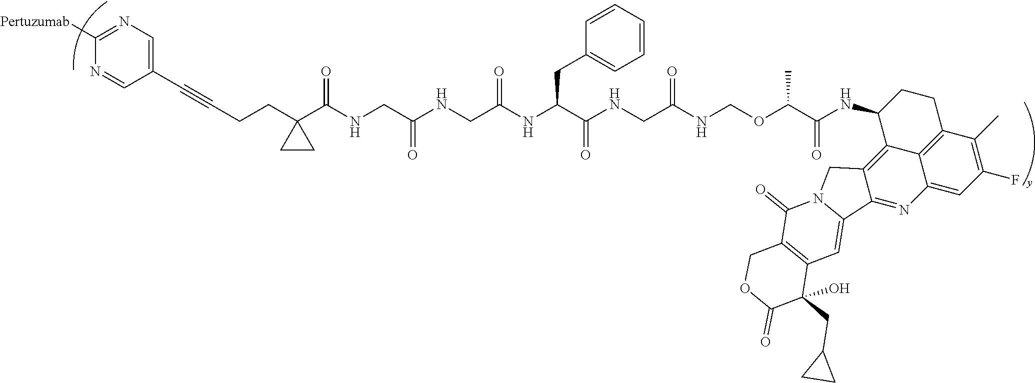

FADC-133

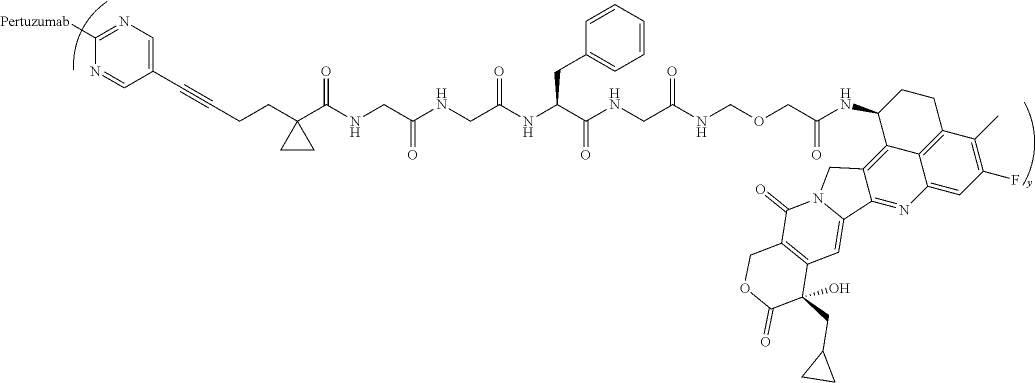

FADC-134

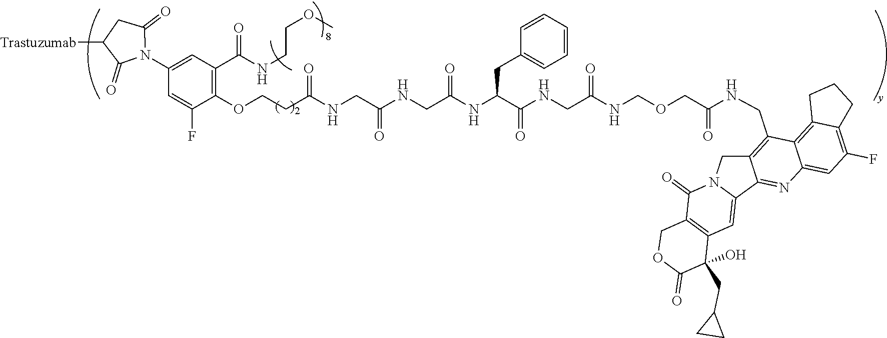

FADC-135

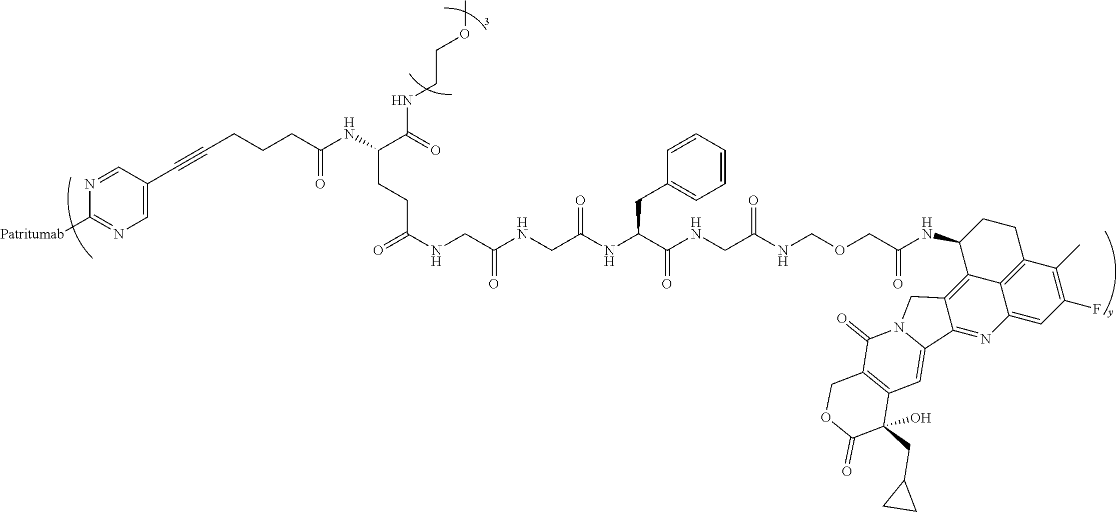

FADC-136

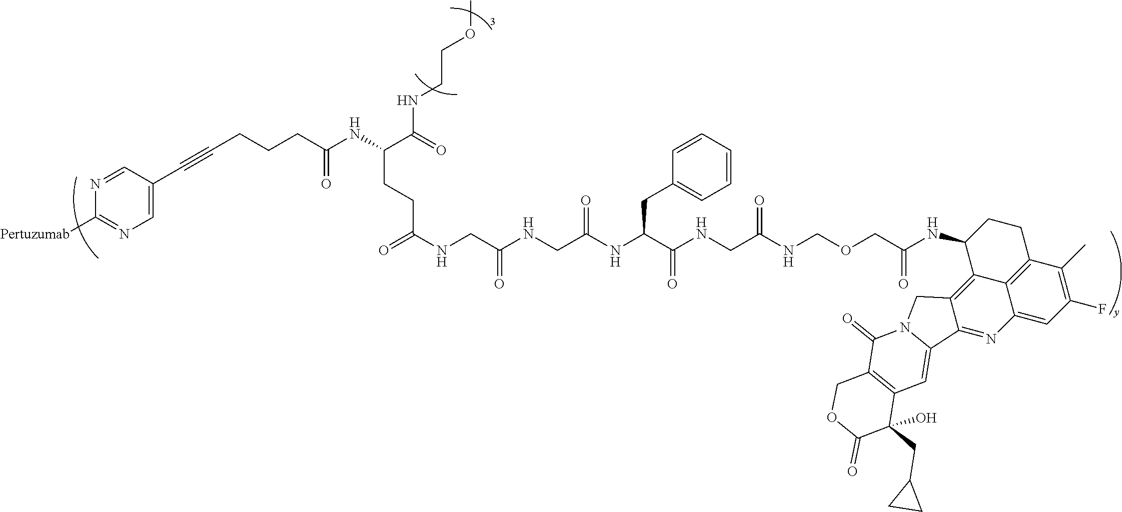

FADC-137

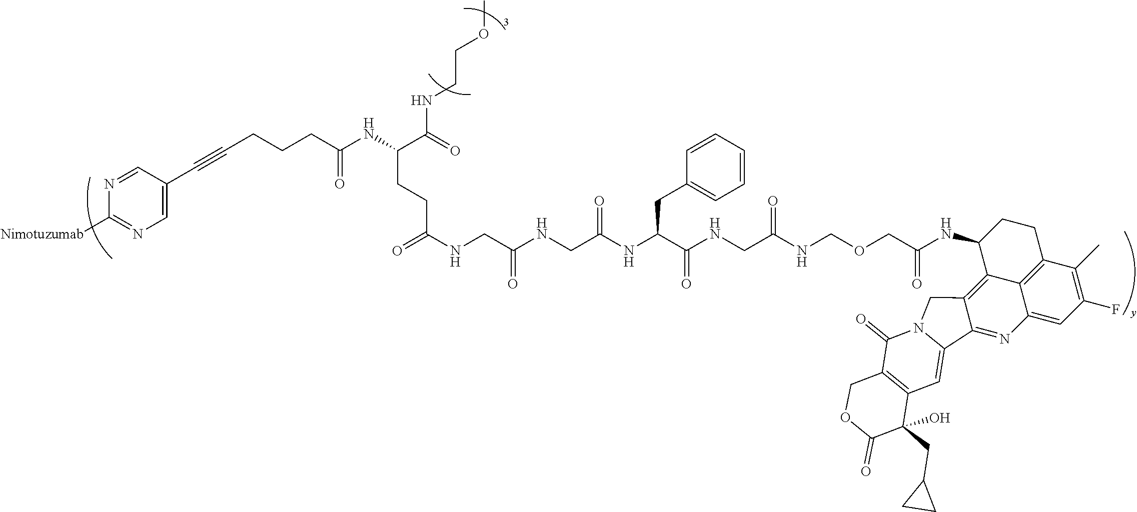

FADC-138

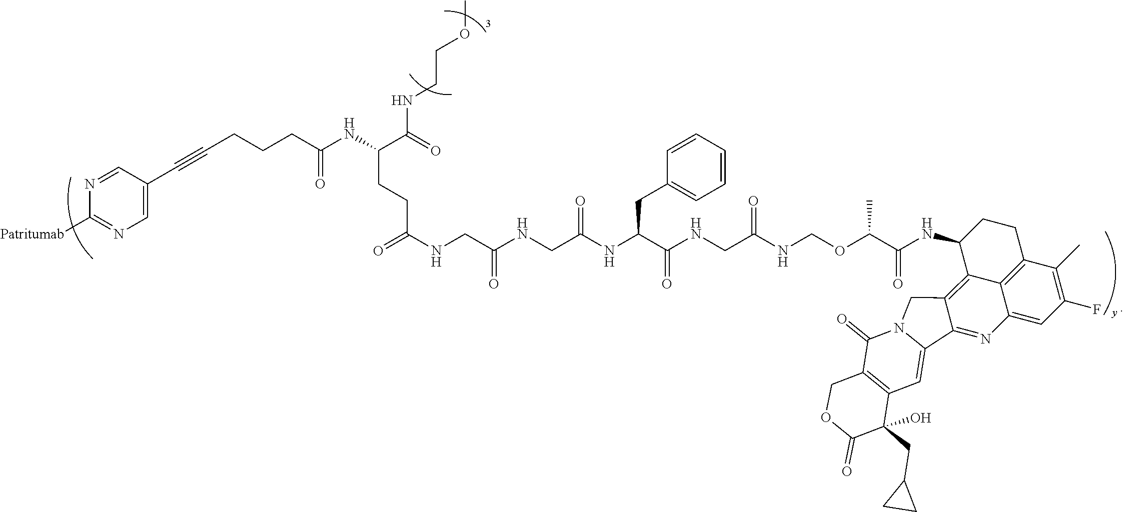

FADC-139

The present invention also provides a method for preparing the conjugate, or a stereoisomer, racemate, tautomer, isotopologue, isotope-labeled compound, nitroxide or pharmaceutically acceptable salt thereof, wherein the preparation method comprises the following steps:
Step 1: providing a linker represented by L^{1′}-L²-L³-L⁴-L^{5′} (L′);
preferably, in the linker,
L⁴-L^{5′} is: —NR^{z1}—C(R^{z2}R^{z3})-T^{L}- in its carboxylic acid form or active ester form;
wherein:
R^{z1} is H or C₁₋₄ alkyl;
R^{z2} and R^{z3} are the same or different and are independently selected from H or C₁₋₄ alkyl;
further preferably:
L⁴-L^{5′} is: —NR^{z1}—C(R²R^{z3})—O—(CH₂)ₘ—C(R^{z4}R^{z5})—CO— in its carboxylic acid form or active ester form;
wherein:
R^{z1} is H or C₁₋₄ alkyl;
m is an integer selected from 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
Step 2: coupling the linker with an intermediate compound represented by Formula (G′) to obtain a coupled intermediate (“linker-drug”) represented by L¹-L²-L³-L⁴-L⁵-G (C′);
wherein, the structure of the intermediate compound as represented by Formula (G′) is:

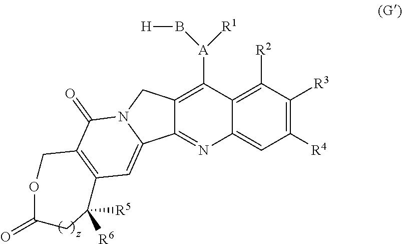

wherein, A, B, T^{L}, R¹, R², R³, R⁴, R⁵, R⁶, L^{1′}, L¹, L², L³, L⁴, L⁵, and L^{5′} independently have the definitions as described above;
preferably, the preparation method further comprises Step 3: coupling the linker-drug represented by Formula (C′) with the targeting moiety Tp;
optionally, if necessary, functional groups of reaction substrates can also be protected with a protecting group known in the art to allow the reaction to proceed, and the protecting group is removed after the reaction.

According to an embodiment of the present invention, the protecting group may be selected from protecting groups known in the art, such as a hydroxy protecting group or an amino protecting group, to allow the reaction to proceed.

The present disclosure also provides a compound obtained by connecting the above-mentioned group G to a hydrogen atom, wherein T^{L} of the group G is connected to the hydrogen atom.

The present disclosure also provides a compound represented by the following Formula (GH), or a stereoisomer, racemate, tautomer, isotopologue, isotope-labeled compound, nitroxide or pharmaceutically acceptable salt thereof:

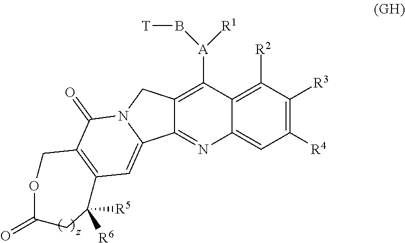

wherein, A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a ring structure which is unsubstituted or substituted with one, two or more R^{E}, for example, the ring structure is a 5- to 10-membered ring structure, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a ring structure which is unsubstituted or substituted with one, two or more R^{F}, for example, the ring structure is a 4- to 10-membered ring structure, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of alkyl, alkyloxy, halogen, hydroxyl, sulfhydryl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy, alkyl, alkyloxy, aryl, arylalkyl, aryloxy, heteroaryl, heteroarylalkyl, heteroaryloxy, amino;
B is absent or selected from the group consisting of triazolyl, —NR⁷— or —N(NR⁷R⁸)—;
R⁷ and R⁸ are the same or different and are independently selected from the group consisting of hydrogen and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a ring structure which is unsubstituted or substituted with one, two or more R^{I}, for example, the ring structure is a 3- to 10-membered ring structure; or, R¹¹ and R¹², together with the atoms to which they are attached, form a ring structure which is unsubstituted or substituted with one, two or more R^{I}, for example, the ring structure is a 3- to 10-membered ring structure; or, R⁹ and R¹¹, together with the atoms to which they are attached, form a ring structure which is unsubstituted or substituted with one, two or more R^{I}, for example, the ring structure is a 3- to 10-membered ring structure; wherein any of the ring structures can be a monocyclic hydrocarbyl, bicyclic hydrocarbyl, heteromonocyclic hydrocarbyl, or heterobicyclic hydrocarbyl, each of which is unsubstituted or substituted with one, two or more R^{I}; for example, any of the ring structures can be the following group which is unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
each m is the same or different, and is independently selected from an integer of 0 to 10;
each n is the same or different, and is independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, aryloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
provided that, when R⁶ is selected from the group consisting of alkyl, alkyloxy, aryl, arylalkyl, aryloxy, heteroaryl, heteroarylalkyl, heteroaryloxy or amino, each of which is unsubstituted or substituted with one, two or more R^{G}:
A is N; R¹ and R², together with the atoms to which they are attached, do not form a 5- to 10-membered ring structure which is unsubstituted or substituted with one, two or more R^{E}; and T^{L} is not a chemical bond when R⁷ is hydrogen;
or, R¹ and R², together the atoms to which they are attached form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl; and R^{A} is selected from the following groups which are unsubstituted or substituted with one, two or more R^{C}: cycloalkyl, cycloalkylalkyl, cycloalkyloxy;
or, B is —N(NR⁷R⁸)—;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl.

According to an embodiment of the present invention, definitions of the groups in Formula (GH) may be independently selected from:
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, or heterobicyclic hydrocarbyl;
or R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, or heterobicyclic hydrocarbyl;
R⁴ is selected from the group consisting of alkyl, alkyloxy, halogen, hydroxyl, sulfhydryl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from the group consisting of triazolyl, —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached with L, and * represents the site attached with B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl alkyl, heteroaryl, heteroarylalkyl, heterocyclyl, heterocycloalkyl;
m and n are the same or different and are independently selected from an integer of 0 to 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—.

According to an embodiment of the present invention, definitions of the groups in Formula (GH) may be independently selected from:
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxy alkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from alkyl, alkyloxy, halogen, hydroxyl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
m and n are the same or different, and are independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different, and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—.

According to an embodiment of the present invention, in the Formula (GH) described in the context, the hydrogen, carbon or other atoms in the formula are optionally replaced by its isotopes, regardless of whether the group is defined as being selected from the above definitions or directly indicated in the formula, and regardless of whether the group is defined as a substituted group or an unsubstituted group. For example, the hydrogen in any group of the formula (GH) can be selected from ¹H, ²H or ³H. As an example, any hydrogen in the substituent of the unsubstituted or substituted alkyl, alkylene, alkylidene, phenyl, pyridyl or lactone group or on the ring-forming atoms (if any) in the compound of Formula (GH) can be independently selected from ¹H, ²H or ³H.

According to an embodiment of the present invention, definitions of the groups in Formula (GH) may be independently selected from:
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)ₙH—;
or, R^{A} and R¹, together with the atom to which they are attached, form a 3- to 8-membered ring structure which is unsubstituted or substituted with one, two or more R^{D}, for example, the 3- to 8-membered ring structure is 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is selected from hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy;
preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to an embodiment of the present invention, definitions of the groups in Formula (GH) may be independently selected from:
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
or, R^{A} and R¹, together with the atom to which they are attached, form a 3- to 8-membered ring structure which is unsubstituted or substituted with one, two or more R^{D}, for example, the 3- to 8-membered ring structure is a 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
provided that, R¹, R² and the atoms to which they are attached, or R², R³ and the atoms to which they are attached, at least one of these two sets of groups together form a 4- to 10-membered ring structure which is unsubstituted or substituted with one, two or more R^{E}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is selected from hydroxyl;
R⁶ is selected from the following groups which are unsubstituted or substituted with one, two or more R^{G}: C₁₋₁₀ alkyl, C₁₋₁₀ alkyloxy, C₆₋₁₀ aryl, C₆₋₁₀ arylalkyl, C₆₋₁₀ aryloxy, 5- to 6-membered heteroaryl, 5- to 6-membered heteroarylalkyl, 5- to 6-membered heteroaryloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein, any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, amino, nitro, the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different, and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to an embodiment of the present invention, definitions of the groups in Formula (GH) may be independently selected from:
A is selected from N;
R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is selected from hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy;
preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is selected from —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; any of the 3- to 10-membered ring structures can be, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

According to the embodiments of the present disclosure, R¹ and R², together with the quinolyl to which they are connected, form one of the following substructures, wherein the substructures may be unsubstituted or substituted with one, two or more R^{E}:

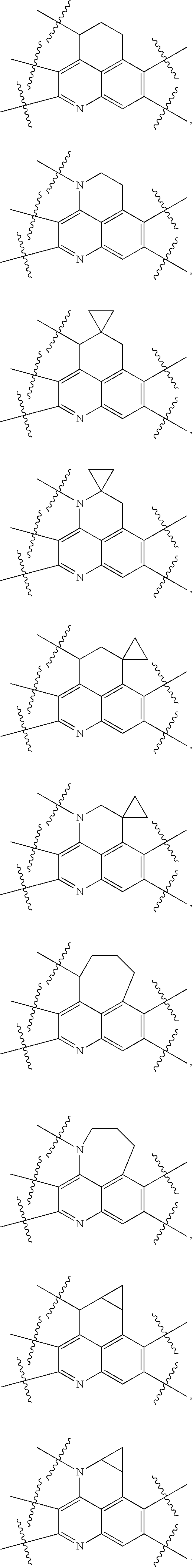

Alternatively, according to the embodiments of the present disclosure, R² and R³, together with the quinolyl to which they are connected, form one of the following substructures, wherein the substructures may be unsubstituted or substituted with one, two or more R^{F}:

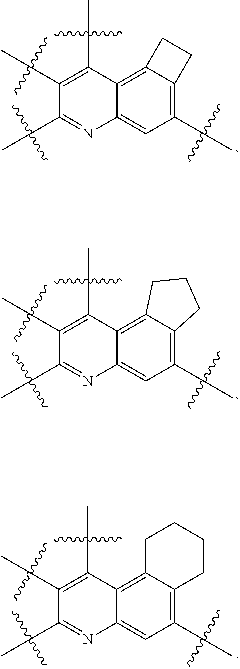 [SMILES: *c1cc2nc(*)c(*)c(*)c2c2c1CC2 ; *c1cc2nc(*)c(*)c(*)c2c2c1CCC2 ; *c1cc2nc(*)c(*)c(*)c2c2c1CCCC2]

According to the embodiments of the present disclosure, B is absent or selected from the group consisting of

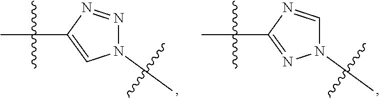 [SMILES: *c1cn(*)nn1 ; *c1ncn(*)n1]

—NH— or —N(NR⁷R⁸)—, wherein R⁷ and R⁸ independently have the definitions as described above.

According to an embodiment of the present disclosure, the compound represented by Formula (GH) may be selected from the compound represented by Formula (GH-1):

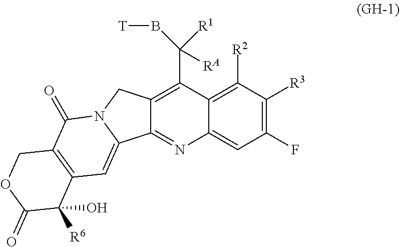

wherein, R^{A}, B, T, R¹, R², R³, and R⁶ are independently defined as described above.

According to an embodiment of the present disclosure, the compound represented by Formula (GH) may be selected from the compound represented by Formula (GH-2) or (GH-3):

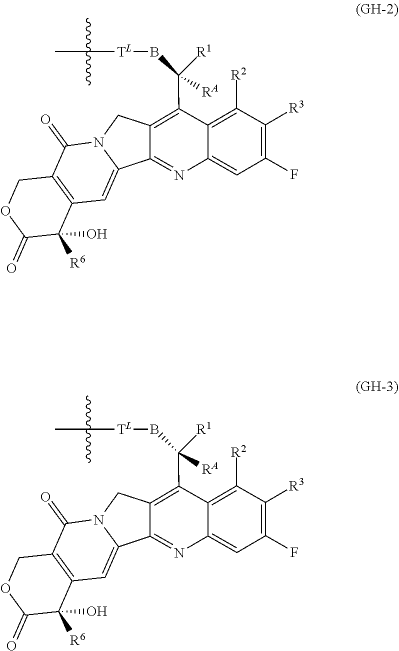

wherein, R^{A}, B, T, R¹, R², R³, and R⁶ are independently defined as described above.

According to the embodiment of the present disclosure, the compound represented by Formula (GH) can be selected from the following compounds:

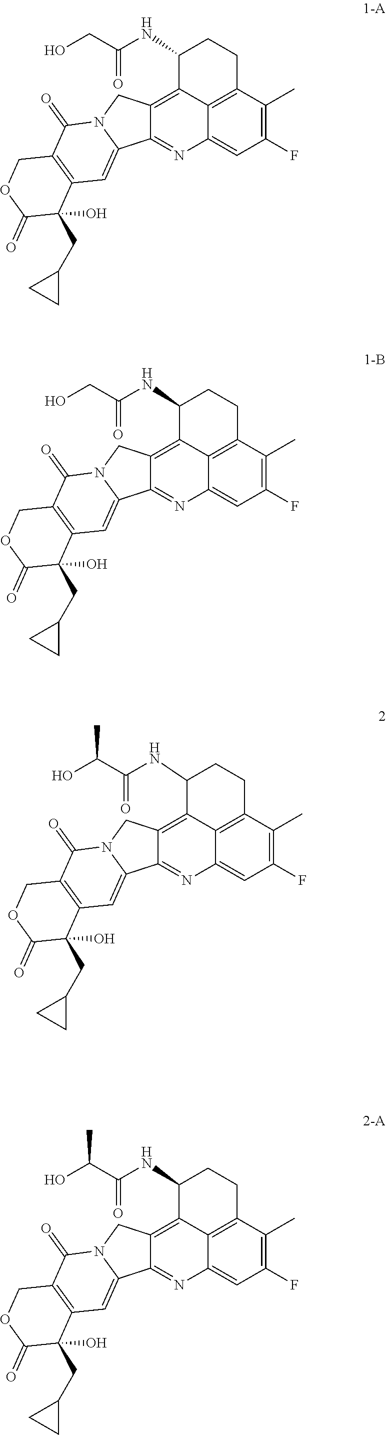 [SMILES: Cc1c(F)cc2nc3c(c4c2c1CC[C@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@]2(O)CC1CC1 ; Cc1c(F)cc2nc3c(c4c2c1CCC4NC(=O)[C@H](C)O)Cn1c-3cc2c(c1=O)COC(=O)[C@]2(O)CC1CC1 ; Cc1c(F)cc2nc3c(c4c2c1CC[C@@H]4NC(=O)[C@H](C)O)Cn1c-3cc2c(c1=O)COC(=O)[C@]2(O)CC1CC1]

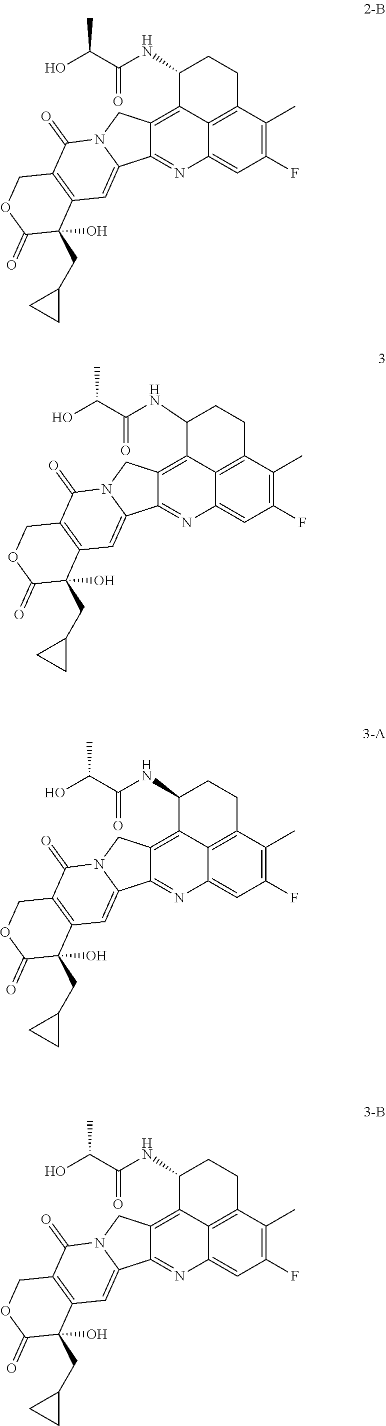 [SMILES: Cc1c(F)cc2nc3c(c4c2c1CC[C@H]4NC(=O)[C@H](C)O)Cn1c-3cc2c(c1=O)COC(=O)[C@]2(O)CC1CC1 ; Cc1c(F)cc2nc3c(c4c2c1CCC4NC(=O)[C@@H](C)O)Cn1c-3cc2c(c1=O)COC(=O)[C@]2(O)CC1CC1 ; Cc1c(F)cc2nc3c(c4c2c1CC[C@@H]4NC(=O)[C@@H](C)O)Cn1c-3cc2c(c1=O)COC(=O)[C@]2(O)CC1CC1 ; Cc1c(F)cc2nc3c(c4c2c1CC[C@H]4NC(=O)[C@@H](C)O)Cn1c-3cc2c(c1=O)COC(=O)[C@]2(O)CC1CC1]

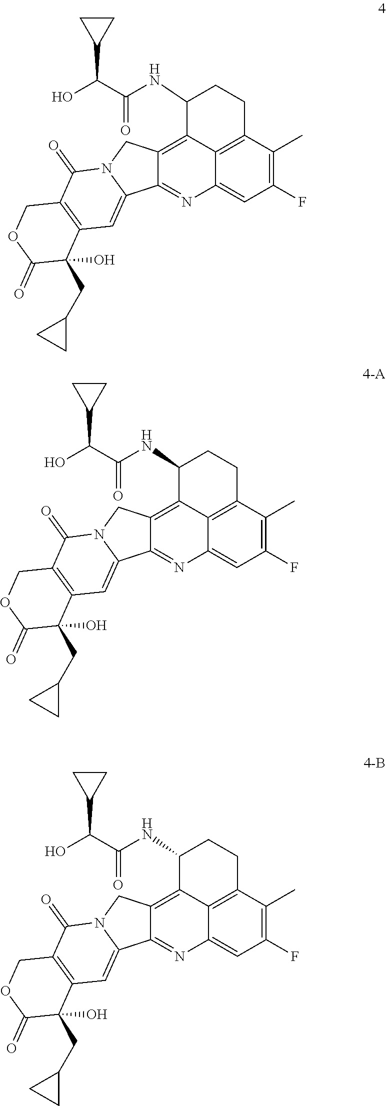

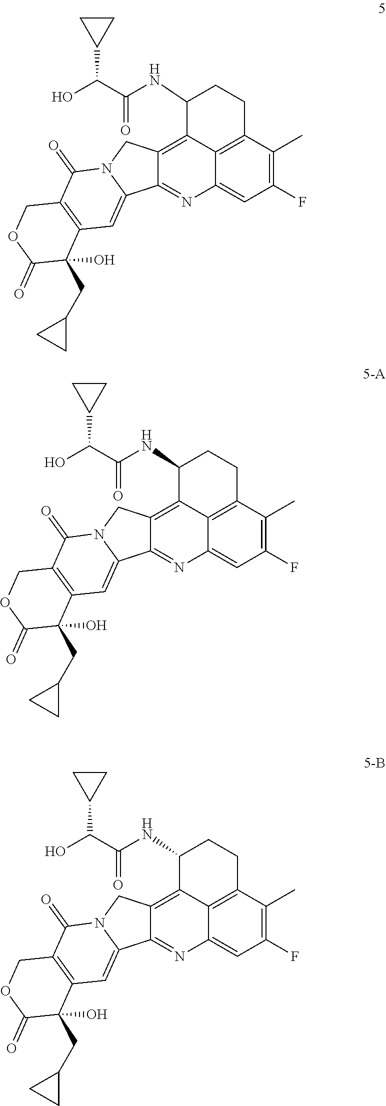

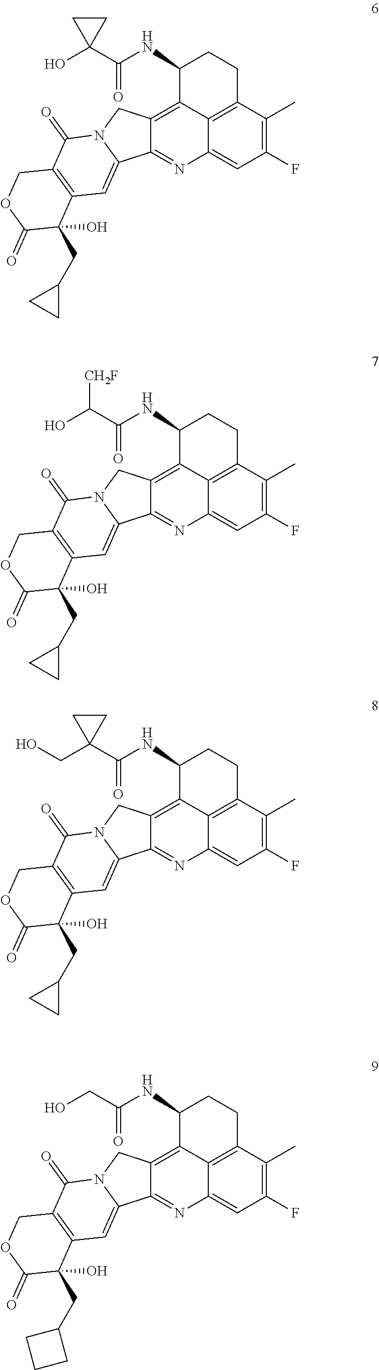

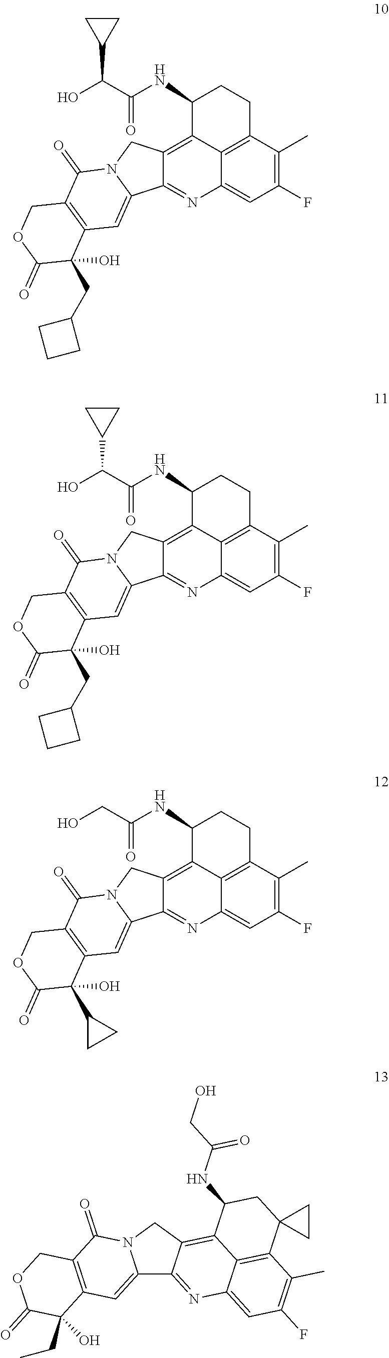 [SMILES: Cc1c(F)cc2nc3c(c4c2c1CC[C@@H]4NC(=O)[C@@H](O)C1CC1)Cn1c-3cc2c(c1=O)COC(=O)[C@]2(O)CC1CCC1 ; Cc1c(F)cc2nc3c(c4c2c1CC[C@@H]4NC(=O)[C@H](O)C1CC1)Cn1c-3cc2c(c1=O)COC(=O)[C@]2(O)CC1CCC1 ; Cc1c(F)cc2nc3c(c4c2c1CC[C@@H]4NC(=O)CO)Cn1c-3cc2c(c1=O)COC(=O)[C@]2(O)C1CC1 ; CC[C@@]1(O)C(=O)OCc2c1cc1n(c2=O)Cc2c-1nc1cc(F)c(C)c3c1c2[C@@H](NC(=O)CO)CC31CC1]

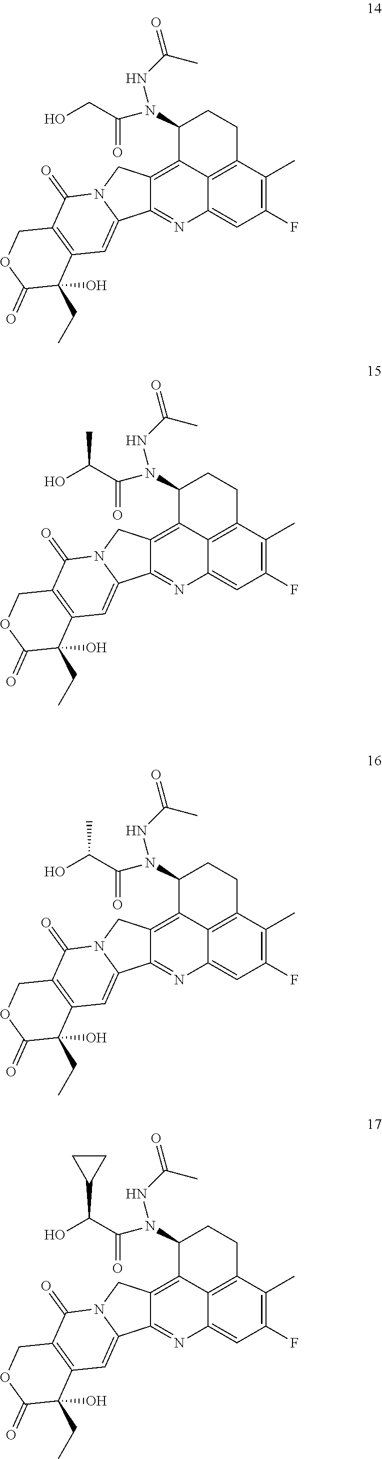 [SMILES: CC[C@@]1(O)C(=O)OCc2c1cc1n(c2=O)Cc2c-1nc1cc(F)c(C)c3c1c2[C@@H](N(NC(C)=O)C(=O)CO)CC3 ; CC[C@@]1(O)C(=O)OCc2c1cc1n(c2=O)Cc2c-1nc1cc(F)c(C)c3c1c2[C@@H](N(NC(C)=O)C(=O)[C@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](O)C1CC1)CC3]

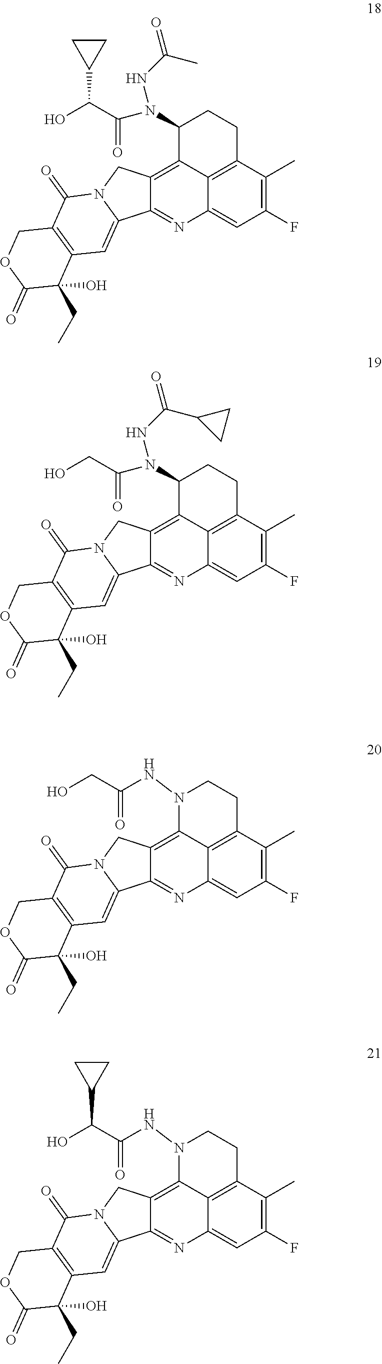 [SMILES: CC[C@@]1(O)C(=O)OCc2c1cc1n(c2=O)Cc2c-1nc1cc(F)c(C)c3c1c2[C@@H](N(NC(C)=O)C(=O)[C@H](O)C1CC1)CC3 ; CC[C@@]1(O)C(=O)OCc2c1cc1n(c2=O)Cc2c-1nc1cc(F)c(C)c3c1c2[C@@H](N(NC(=O)C1CC1)C(=O)CO)CC3 ; CC[C@@]1(O)C(=O)OCc2c1cc1n(c2=O)Cc2c-1nc1cc(F)c(C)c3c1c2N(NC(=O)CO)CC3 ; CC[C@@]1(O)C(=O)OCc2c1cc1n(c2=O)Cc2c-1nc1cc(F)c(C)c3c1c2N(NC(=O)[C@@H](O)C1CC1)CC3]

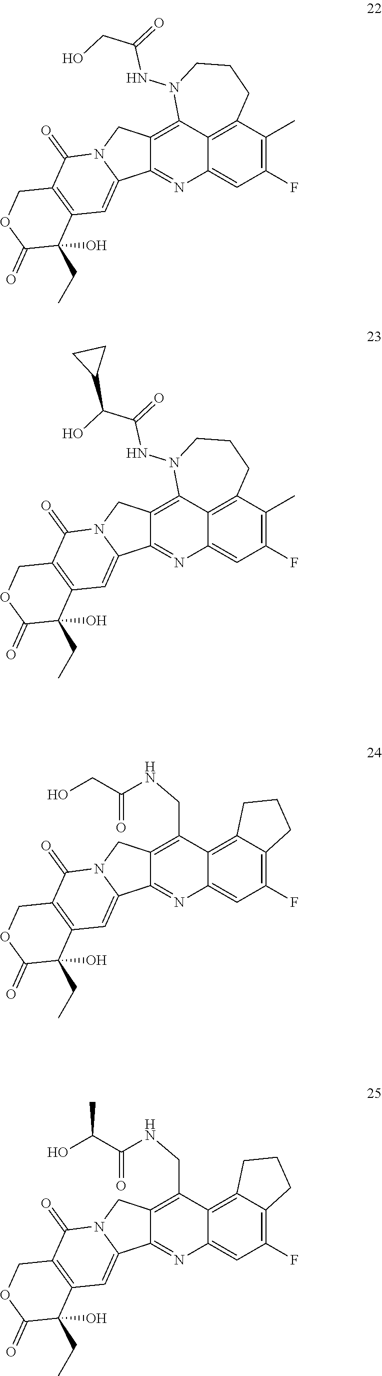 [SMILES: CC[C@@]1(O)C(=O)OCc2c1cc1n(c2=O)Cc2c-1nc1cc(F)c(C)c3c1c2N(NC(=O)CO)CCC3 ; CC[C@@]1(O)C(=O)OCc2c1cc1n(c2=O)Cc2c-1nc1cc(F)c(C)c3c1c2N(NC(=O)[C@@H](O)C1CC1)CCC3 ; CC[C@@]1(O)C(=O)OCc2c1cc1n(c2=O)Cc2c-1nc1cc(F)c3c(c1c2CNC(=O)CO)CCC3 ; CC[C@@]1(O)C(=O)OCc2c1cc1n(c2=O)Cc2c-1nc1cc(F)c3c(c1c2CNC(=O)[C@H](C)O)CCC3]

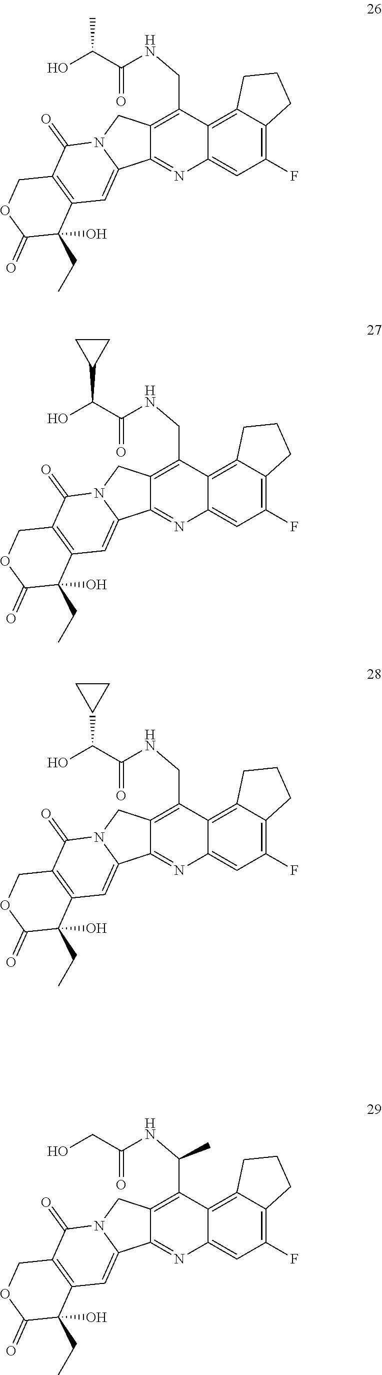

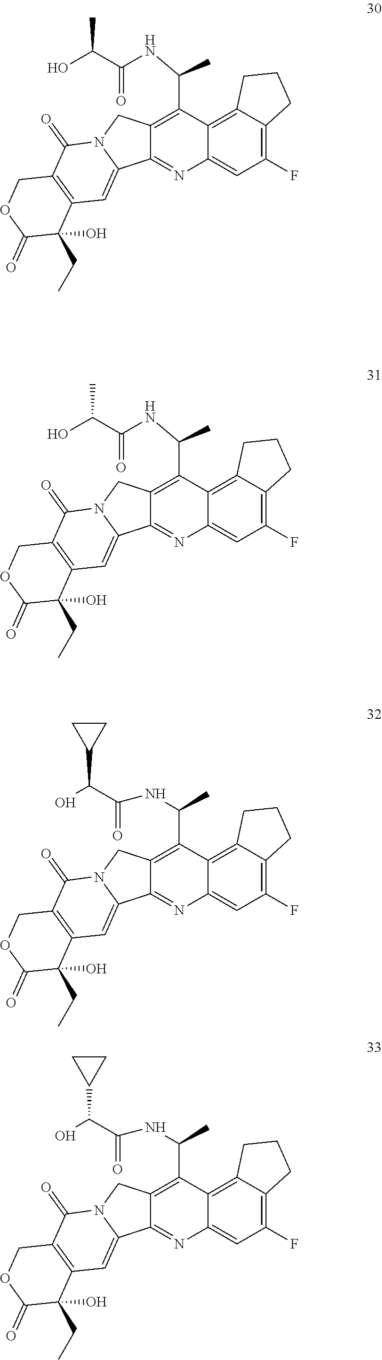

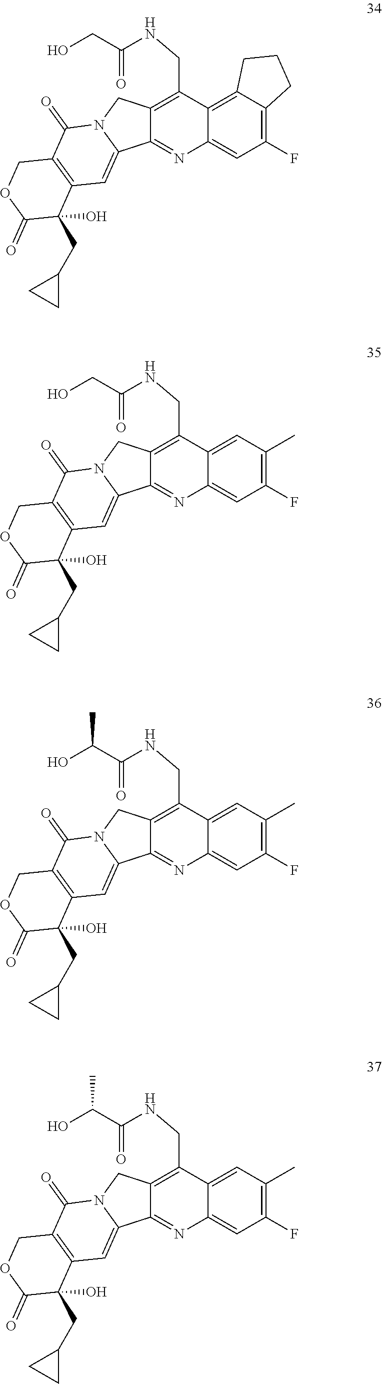 [SMILES: O=C(CO)NCc1c2c(nc3cc(F)c4c(c13)CCC4)-c1cc3c(c(=O)n1C2)COC(=O)[C@]3(O)CC1CC1 ; Cc1cc2c(CNC(=O)CO)c3c(nc2cc1F)-c1cc2c(c(=O)n1C3)COC(=O)[C@]2(O)CC1CC1 ; Cc1cc2c(CNC(=O)[C@H](C)O)c3c(nc2cc1F)-c1cc2c(c(=O)n1C3)COC(=O)[C@]2(O)CC1CC1 ; Cc1cc2c(CNC(=O)[C@@H](C)O)c3c(nc2cc1F)-c1cc2c(c(=O)n1C3)COC(=O)[C@]2(O)CC1CC1]

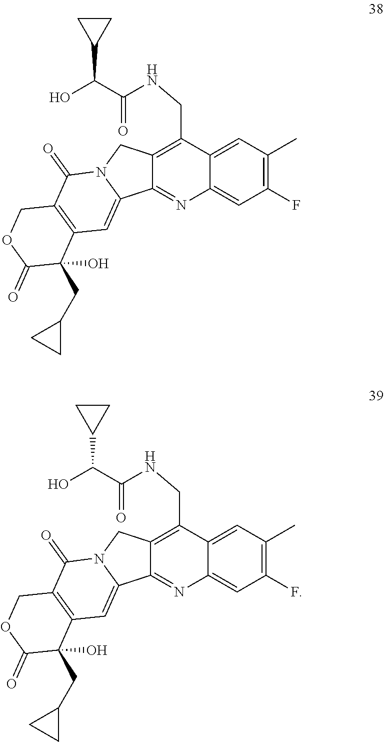 [SMILES: Cc1cc2c(CNC(=O)[C@@H](O)C3CC3)c3c(nc2cc1F)-c1cc2c(c(=O)n1C3)COC(=O)[C@]2(O)CC1CC1 ; Cc1cc2c(CNC(=O)[C@H](O)C3CC3)c3c(nc2cc1F)-c1cc2c(c(=O)n1C3)COC(=O)[C@]2(O)CC1CC1]

The present disclosure also provides a method for preparing the compound represented by Formula (GH), or a stereoisomer, racemate, tautomer, isotopologue, isotope-labeled compound, nitroxide or pharmaceutically acceptable salt thereof, the method comprises a step of reacting the compound of Formula (i) with the compound of Formula (ii):

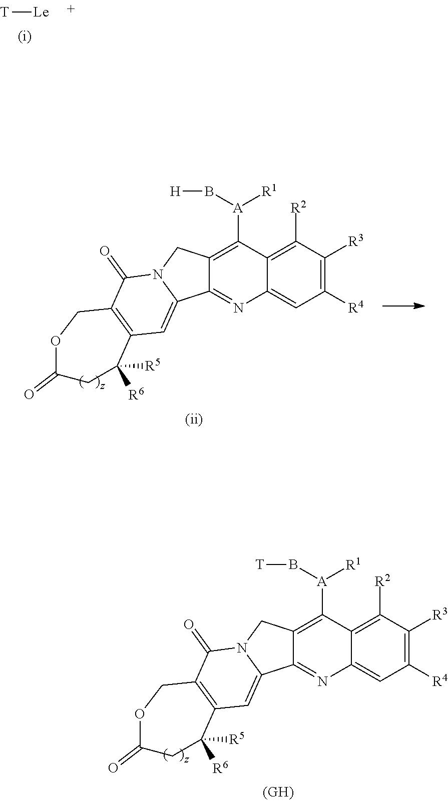

wherein, A, B, T, R¹, R², R³, R⁴, R⁵, R⁶, and z independently have the definitions described above;
LE represents a group that is removed or leaves after the reaction.

The present invention also provides a method for preparing a compound of Formula (ii), comprising a step of reacting a compound of Formula (iii) with a compound of Formula (iv) to obtain a compound of Formula (v):

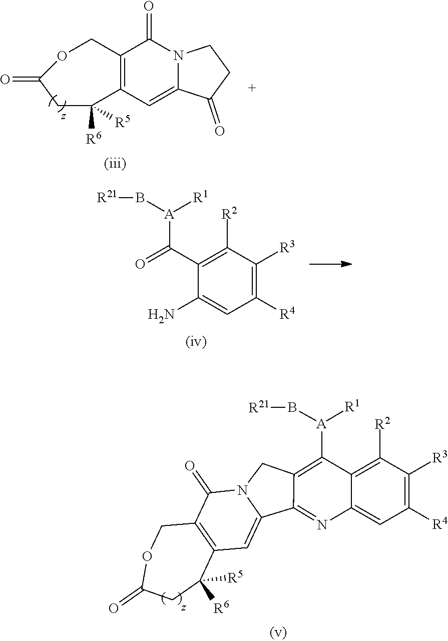

wherein, A, B, R, R², R³, R⁴, R⁵, R⁶, and z independently have the definitions described above;
R²¹ is selected from H or a protecting group.

According to an embodiment of the present invention, when R²¹ is selected from H, the compound of Formula (v) is the compound of Formula (ii).

According to an embodiment of the present invention, when R²¹ is selected from a protecting group, the compound of Formula (v) may be reacted under conditions of deprotection after obtaining the compound of Formula (v), to obtain the compound of Formula (ii).

According to an embodiment of the present invention, the protecting group is selected from an amino protecting group.

According to an embodiment of the present invention, the type of the protecting group (e.g., an amino protecting group) and the conditions for deprotection may be selected from types or conditions known to those skilled in the art.

The present disclosure also provides a compound represented by Formula (iii):

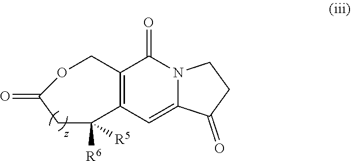

wherein, R⁵, R⁶, and z independently have the definitions described above.

The present disclosure also provides a compound represented by Formula (v):

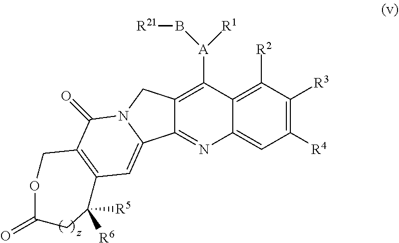

wherein, A, B, R¹, R², R³, R⁴, R⁵, R⁶, R²¹, and z independently have the definitions described above.

The present disclosure also provides a pharmaceutical composition, which comprises at least one selected from the following: the compound represented by Formula (GH), or a stereoisomer, racemate, tautomer, isotopologue, isotope-labeled compound, nitroxide, or pharmaceutically acceptable salt thereof, the conjugate represented by Formula (C), or a stereoisomer, racemate, tautomer, isotopologue, isotope-labeled compound, nitroxide, or pharmaceutically acceptable salt thereof.

Preferably, if present, the compound represented by Formula (GH), or a stereoisomer, racemate, tautomer, isotopologue, isotope-labeled compound, nitroxide or pharmaceutically acceptable salt thereof, or the conjugate represented by Formula (C), or a stereoisomer, racemate, tautomer, isotopologue, isotope-labeled compound, nitroxide or pharmaceutically acceptable salt thereof in the pharmaceutical composition is present in a therapeutically effective amount.

According to an embodiment of the present disclosure, the pharmaceutical composition comprises a therapeutically effective amount of at least one selected from the following: the compound represented by Formula (GH), or a stereoisomer, racemate, tautomer, isotopologue, isotope-labeled compound, nitroxide or pharmaceutically acceptable salt thereof, the conjugate represented by Formula (C), or a stereoisomer, racemate, tautomer, isotopologue, isotope-labeled compound, nitroxide or pharmaceutically acceptable salt thereof.

The present disclosure also provides use of the compound represented by the Formula (GH), or a stereoisomer, racemate, tautomer, isotopologue, isotope-labeled compound, nitroxide or pharmaceutically acceptable salt thereof, or the conjugate represented by the Formula (C), or a stereoisomer, racemate, tautomer, isotopologue, isotope-labeled compound, nitroxide or pharmaceutically acceptable salt thereof, for preventing and/or treating a disease or disorder, and/or in the manufacture of a medicament.

According to an embodiment of the present disclosure, the medicament is used for preventing and/or treating a disease or disorder.

The present disclosure also provides a method for preventing and/or treating a disease or disorder, the method comprising administering to a patient a therapeutically effective amount of at least one of the following: the compound represented by the Formula (GH), or a stereoisomer, racemate, tautomer, isotopologue, isotope-labeled compound, nitroxide or pharmaceutically acceptable salt thereof, the conjugate represented by the Formula (C), or a stereoisomer, racemate, tautomer, isotopologue, isotope-labeled compound, nitroxide or pharmaceutically acceptable salt thereof.

According to an embodiment of the present disclosure, the disease or disorder can be selected from a tumor, such as a solid tumor or a hematological cancer.

Examples of the solid tumor include malignancies of various organ systems, such as sarcomas, adenocarcinomas, blastomas, and carcinomas, such as those affecting liver, lung, breast, lymph, biliary-intestinal (e.g., colon), genitourinary tract (e.g., kidney, urothelial cells), prostate, and pharynx. Adenocarcinomas include malignancies such as most colon cancers, rectal cancers, renal cell carcinomas, hepatocarcinomas, small cell lung cancers, non-small cell lung cancers, small intestinal cancers, and esophageal cancers. In one embodiment, the cancer is melanoma, such as advanced melanoma. Examples of other cancers that may be treated include: bone cancer, pancreatic cancer, skin cancer, head and neck cancer, malignant melanoma of skin or in eyes, uterine cancer, ovarian cancer, rectal cancer, colorectal cancer, cancer of anal region, cancer of peritoneum, stomach cancer, esophageal cancer, salivary gland cancer, testicular cancer, fallopian tube cancer, endometrial cancer, cervical cancer, vaginal cancer, vulvar cancer, penile cancer, malignant glioma, neuroblastoma, Hodgkin's disease, non-Hodgkin's lymphoma, esophageal cancer, small intestine cancer, cancer of endocrine system, thyroid cancer, parathyroid cancer, adrenal cancer, soft tissue sarcoma, urethral cancer, chronic or acute leukemia (including acute myeloid leukemia, chronic myeloid leukemia, acute lymphoblastic leukemia, chronic lymphocytic leukemia), solid tumors of children, lymphocytic lymphoma, bladder cancer, kidney cancer or ureter cancer, renal pelvic cancer, central nervous system (CNS) tumor, primary CNS lymphoma, tumor angiogenesis, spinal cord axis tumor, brain stem glioma, pituitary adenoma, Kaposi's sarcoma, neuroendocrine tumor (including carcinoid tumor, gastrinoma, and islet cell carcinoma), mesothelioma, schwannoma (including acoustic neuroma), meningioma, epidermoid carcinoma, squamous cell carcinoma, T-cell lymphoma, environmentally induced cancer (including cancer induced by asbestos), and any combination of the above cancers.

Examples of the above hematological cancers include leukemias, lymphomas, and malignant lymphoproliferative disorders that affect the blood, bone marrow, and lymphatic system. Leukemias can be classified into acute leukemias and chronic leukemias. Acute leukemias can be further classified into acute myeloid leukemia (AML) and acute lymphocytic leukemia (ALL). Chronic leukemias include chronic myeloid leukemia (CML) and chronic lymphocytic leukemia (CLL). Other related conditions include myelodysplastic syndrome (MDS, formerly known as “preleukemia”), which is a diverse collection of hematological conditions associated with ineffective production (or dysplasia) of bone marrow blood cells and the risk of transformation to AML. Lymphomas are a group of blood cell tumors that develop from lymphocytes. Exemplary lymphomas include non-Hodgkin lymphoma and Hodgkin lymphoma.

According to an embodiment of the present disclosure, the pharmaceutical composition may also comprise a pharmaceutically acceptable excipient, such as a carrier or excipient. The pharmaceutically acceptable excipient is preferably chemically non-reactive or inert to the active ingredient. For example, the pharmaceutically acceptable excipient is at least one selected from the following excipients, including but not limited to: fillers, disintegrants, binders, lubricants, surfactants, flavoring agents, wetting agents, matrix, etc.

According to the embodiments of the present disclosure, the administration route of the pharmaceutical composition includes but is not limited to gastrointestinal administration or parenteral administration; wherein the gastrointestinal administration may be oral administration; the parenteral administration may be topical administration, transdermal administration, injection administration, etc.

In one embodiment, the administration route of the pharmaceutical composition may be topical administration combined with oral administration.

According to the embodiments of the present disclosure, the dosage form of the pharmaceutical composition may be selected from capsules, tablets, patches, films, pills, powders, lozenges, sachets, cachets, elixirs, suspensions, emulsions, solutions, syrups, aerosols, ointments, creams, suppositories or injections.

### Definitions and Explanations of Terms

Unless otherwise specified, definitions of the terms described in the description and claims of the present application, including those provided as examples, exemplary or preferred, in tables, and in the specific compounds of the examples, etc., may be arbitrarily combined and integrated with each other. Such combination and integration shall fall within the scope of the description of the present application.

Unless otherwise specified, the numerical ranges recorded in the present description and claims are equivalent to recording at least each specific integer value therein. For example, the numerical range “1 to 10” is equivalent to recording each integer value in the numerical range “1 to 10”, namely 1, 2, 3, 4, 5, 6, 7, 8, 9, 10. It should be understood that when “one”, “two” or “more” are used herein to describe substituents, “more” should refer to an integer ≥3, such as 3, 4, 5, 6, 7, 8, 9 or 10. In addition, when certain numerical ranges are defined as “numbers”, it should be understood that the two endpoints of the range, each integer in the range, and each decimal in the range are recorded. For example, “a number from 0 to 10” should be understood as not only recording each integer of 0, 1, 2, 3, 4, 5, 6, 7, 8, 9 and 10, but also at least recording the sum of each integer therein and 0.1, 0.2, 0.3, 0.4, 0.5, 0.6, 0.7, 0.8, 0.9, respectively. Similarly, “an integer or decimal of 1 to 50”, “an integer or decimal of 1 to 20”, “an integer or decimal of 1 to 10” should be understood as recording the two endpoints of each of the above numerical ranges, as well as each integer in each range and each decimal in the range.

The term “isotopologue” comprises replacing an atom in a compound or conjugate of the present disclosure with other isotopes having the same atomic number but different atomic mass or mass number. For example, in the compound or conjugate of the present disclosure, “hydrogen” can be selected from protium (¹H), deuterium (²H) and tritium (³H); “carbon” can be selected from ¹²C, ¹³C and ¹⁴C. Therefore, the compound or conjugate of the present disclosure should at least be understood as compounds or conjugates containing various deuterated forms. For example, one, two or more of available hydrogen atoms connected to a carbon atom (e.g., hydrogen atoms of C₁₋₁₀ alkyl) can be independently replaced by deuterium atoms. Those skilled in the art can synthesize deuterated compounds or conjugates with reference to relevant literatures. When preparing deuterated compounds or conjugates, commercially available deuterated starting materials can be used, or deuterated reagents can be used for synthesis using conventional techniques. Optional deuterated reagents include, but are not limited to, deuterated borane, trideuterated borane in tetrahydrofuran solution, deuterated lithium aluminum hydride, deuterated iodoethane and deuterated iodomethane.

The term “isotope-labeled compound” includes, but is not limited to, the compound or conjugate of the present disclosure that is labeled with at least one of the following elements: ¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷a, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²²⁷Th and ²¹¹Pb.

The term “halogen” refers to fluorine, chlorine, bromine and iodine.

The term “alkyl” should be understood to preferably refer to a straight or branched saturated monovalent hydrocarbyl, preferably “C₁₋₁₀ alkyl”. “C₁₋₁₀ alkyl” should be understood to preferably refer to a straight or branched saturated monovalent hydrocarbyl having 1 to 10 carbon atoms. For example, “C₁₋₆ alkyl” refers to a straight and branched alkyl having 1, 2, 3, 4, 5 or 6 carbon atoms. The alkyl is, for example, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or 1,2-dimethylbutyl, etc. or isomers thereof.

The term “cycloalkyl” should be understood to refer to a saturated monovalent monocyclic or bicyclic (fused, bridged or spiro ring) hydrocarbyl, preferably “C₃₋₁₀ cycloalkyl”. The term “C₃₋₁₀ cycloalkyl” should be understood to refer to a saturated monovalent monocyclic or bicyclic (fused, bridged or spiro ring) hydrocarbyl having 3 to 10 carbon atoms, such as 3, 4, 5, 6, 7, 8, 9 or 10 carbon atoms. The C₃₋₁₀ cycloalkyl may be a monocyclic hydrocarbyl, such as cyclopropyl, cyclobutyl, cyclopentyl, cyclohexyl, cycloheptyl, cyclooctyl, cyclononyl or cyclodecyl, or a bicyclic hydrocarbyl such as decalin ring.

The term “ring structure” should be understood to refer to a group having a monocyclic, bicyclic (fused, bridged or spiro ring) or multicyclic ring structure, such as a monocyclic hydrocarbyl, a bicyclic hydrocarbyl, a tricyclic hydrocarbyl, a heteromonocyclic hydrocarbyl, a heterobicyclic hydrocarbyl, a heterotricyclic hydrocarbyl, etc.; and the ring structure may be saturated or unsaturated. The heteromonocyclic hydrocarbyl, heterobicyclic hydrocarbyl, and heterotricyclic hydrocarbyl may contain 1 to 10, preferably 1 to 5 heteroatoms, independently selected from N, O and S, such as 1, 2, 3, 4, 5, 6, 7, 8, 9 or 10 heteroatoms independently selected from N, O and S. The bicyclic ring may be the following ring structure containing no heteroatoms or containing 1, 2 or 3 heteroatoms independently selected from N, O and S: one or two of aryl, heteroaryl, cycloalkyl, heterocyclyl, which are independent or fused to each other, spiro[2.5]ring, spiro[3.3]ring, spiro[4.2]ring, spiro[4.3]ring, spiro[5.2]ring, spiro[5.4]ring, bicyclo[2.1.1], bicyclo[2.2.1], bicyclo[2.2.2], bicyclo[3.2.1], bicyclo[4.1.0], etc.. Alternatively, the ring structure may have 1, 2, 3, 4 or 5 double bonds, such as carbon-carbon double bonds or carbon-nitrogen double bonds.

The term “3- to 10-membered ring structure” should be understood to refer to a group having a monocyclic or bicyclic (fused, bridged or spiro ring) structure with 3, 4, 5, 6, 7, 8, 9 or 10 ring atoms, such as a monocyclic hydrocarbyl, a bicyclic hydrocarbyl, a heteromonocyclic hydrocarbyl or a heterobicyclic hydrocarbyl; and the ring structure may be saturated or unsaturated. The heteromonocyclic hydrocarbyl or heterobicyclic hydrocarbyl may contain 1 to 5, preferably 1 to 3 heteroatoms independently selected from N, O and S, such as 1, 2, 3 heteroatoms independently selected from N, O and S. The bicyclic ring may be the following ring structure containing no heteroatoms or containing 1, 2, or 3 heteroatoms independently selected from N, O, and S: one or two of aryl, heteroaryl, cycloalkyl, heterocyclyl, which are independent or fused to each other, spiro[2.5]ring, spiro[3.3]ring, spiro[4.2]ring, spiro[4.3]ring, spiro[5.2]ring, spiro[5.4]ring, bicyclo[2.1.1], bicyclo[2.2.1], bicyclo[2.2.2], bicyclo[3.2.1], bicyclo[4.1.0], etc. Alternatively, the ring structure may have 1, 2, 3, 4, or 5 double bonds, such as carbon-carbon double bonds or carbon-nitrogen double bonds.

The term “4- to 10-membered ring structure” should be understood to refer to a group having a monocyclic or bicyclic (fused, bridged or spiro ring) structure with 4, 5, 6, 7, 8, 9 or 10 ring atoms, such as a monocyclic hydrocarbyl, a bicyclic hydrocarbyl, a heteromonocyclic hydrocarbyl or a heterobicyclic hydrocarbyl; and the ring structure may be saturated or unsaturated. The heteromonocyclic hydrocarbyl or heterobicyclic hydrocarbyl may contain 1 to 5, preferably 1 to 3 heteroatoms independently selected from N, O and S, such as 1, 2, 3 heteroatoms independently selected from N, O and S. The bicyclic ring may be the following ring structure containing no heteroatoms or containing 1, 2, or 3 heteroatoms independently selected from N, O, and S: one or two of aryl, heteroaryl, cycloalkyl, heterocyclyl, which are independent or fused to each other, spiro[2.5]ring, spiro[3.3]ring, spiro[4.2]ring, spiro[4.3]ring, spiro[5.2]ring, spiro[5.4]ring, bicyclo[2.1.1], bicyclo[2.2.1], bicyclo[2.2.2], bicyclo[3.2.1], bicyclo[4.1.0], etc. Alternatively, the ring structure may have 1, 2, 3, 4, or 5 double bonds, such as carbon-carbon double bonds or carbon-nitrogen double bonds.

The term “5- to 10-membered ring structure” should be understood to refer to a group having a monocyclic or bicyclic (fused, bridged or spiro ring) structure with 5, 6, 7, 8, 9 or 10 ring atoms, such as a monocyclic hydrocarbyl, a bicyclic hydrocarbyl, a heteromonocyclic hydrocarbyl or a heterobicyclic hydrocarbyl; and the ring structure may be saturated or unsaturated. The heteromonocyclic hydrocarbyl or heterobicyclic hydrocarbyl may contain 1 to 5, preferably 1 to 3 heteroatoms independently selected from N, O and S, such as 1, 2, 3 heteroatoms independently selected from N, O and S. The bicyclic ring may be the following ring structure containing on heteroatoms or containing 1, 2, or 3 heteroatoms independently selected from N, O, and S: one or two of aryl, heteroaryl, cycloalkyl, heterocyclyl, which are independent or fused to each other, spiro[2.5]ring, spiro[3.3]ring, spiro[4.2]ring, spiro[4.3]ring, spiro[5.2]ring, spiro[5.4]ring, bicyclic[2.1.1], bicyclic[2.2.1], bicyclic[2.2.2], bicyclic[3.2.1], bicyclic[4.1.0], etc.

Alternatively, the “4- to 10-membered ring structure” and “5- to 10-membered ring structure” may have 1, 2, 3, 4, or 5 double bonds, such as carbon-carbon double bonds or carbon-nitrogen double bonds.

It should be understood that when the group of the compound described in the present invention forms the above-mentioned ring structure, it may form a fused ring, spiro ring or bicyclic ring with the original ring structure of the compound, and when there is an unsaturated bond in the original ring structure of the compound, the ring structure formed by the group preferably retains the unsaturated bond in the original ring structure.

The term “heterocyclyl” refers to a saturated or unsaturated monovalent monocyclic, bicyclic (fused, bridged or spiro ring) hydrocarbyl, which contains 1 to 5 heteroatoms independently selected from N, O and S, but is not aromatic. The “heterocyclyl” is preferably a “3- to 20-membered heterocyclyl”. The term “3- to 20-membered heterocyclyl” refers to a saturated monovalent monocyclic, bicyclic (fused, bridged or spiro ring) hydrocarbyl, which contains 1 to 5 heteroatoms independently selected from N, O and S, and the total number of ring atoms is 3 to 20 (e.g., 3, 4, 5, 6, 7, 8, 9, 10, etc.), preferably a “3- to 10-membered heterocyclyl”. The term “3- to 10-membered heterocyclyl” refers to a saturated or unsaturated monovalent monocyclic, bicyclic (fused, bridged or spiro) hydrocarbyl, which contains 1 to 5, preferably 1 to 3 heteroatoms independently selected from N, O and S, such as 1, 2, 3 heteroatoms independently selected from N, O and S, but which is not aromatic. The heterocyclyl may be attached to the rest part of the molecule via any of the carbon atoms or nitrogen atoms (if present). In particular, the heterocyclyl may include, but is not limited to: 4-membered ring, such as azetidinyl, oxetanyl (e.g., azetidin-1-yl); 5-membered ring, such as tetrahydrofuranyl, dioxolyl, pyrrolidinyl, imidazolidinyl, pyrazolidinyl, pyrrolinyl; or 6-membered ring, such as tetrahydropyranyl, piperidinyl, morpholinyl, dithianyl, thiomorpholinyl, piperazinyl or trithianyl; or 7-membered ring, such as diazecycloheptyl. Optionally, the heterocyclyl can be benzo-fused. The heterocyclyl can be bicyclic, for example, but not limited to, 5,5-membered ring, such as hexahydrocyclopenta[c]pyrrol-2(1H)-yl ring, or 5,6-membered bicyclic ring, such as hexahydropyrrolo[1,2-a]pyrazin-2(1H)-yl ring. The ring containing nitrogen atoms can be partially unsaturated, that is, it can contain one or more double bonds, for example, but not limited to, 2,5-dihydro-1H-pyrrolyl, 4H-[1,3,4]thiadiazinyl, 4,5-dihydrooxazolyl or 4H-[1,4]thiazinyl, or, it can be benzo-fused, for example, but not limited to, dihydroisoquinolinyl. According to the present disclosure, the heterocyclyl is non-aromatic. When the 3- to 20-membered heterocyclyl is connected with other groups to form the compound disclosed herein, the carbon atoms on the 3- to 20-membered heterocyclyl may be connected with other groups, or the heterocyclic atoms on the 3- to 20-membered heterocyclyl may be connected with other groups. For example, when the 3- to 20-membered heterocyclyl is selected from piperazinyl, the nitrogen atom on the piperazinyl may be connected with other groups. Alternatively, when the 3- to 20-membered heterocyclyl is selected from piperidinyl, the nitrogen atom on the piperidinyl ring and the carbon atom on the para position may be connected with other groups.

The term “aryl” should be understood as a monocyclic, bicyclic or tricyclic hydrocarbyl with monovalent aromaticity or partial aromaticity, preferably a “C₆₋₂₀ aryl”. The term “C₆₋₂₀ aryl” should be understood to preferably refer to a monocyclic, bicyclic or tricyclic hydrocarbyl with monovalent aromaticity or partial aromaticity having 6 to 20 carbon atoms, preferably a “C₆₋₁₄ aryl”. The term “C₆₋₁₄ aryl” should be understood to preferably refer to a monocyclic, bicyclic or tricyclic hydrocarbyl (“C₆₋₁₄ aryl”) with monovalent aromaticity or partial aromaticity having 6, 7, 8, 9, 10, 11, 12, 13 or 14 carbon atoms, in particular, a ring having 6 carbon atoms (“C₆ aryl”), such as phenyl; or biphenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl or naphthyl, or a ring having 13 carbon atoms (“C₁₃ aryl”), such as fluorenyl, or a ring having 14 carbon atoms (“C₁₄ aryl”), such as anthracenyl. When the C₆₋₂₀ aryl is substituted, it may be monosubstituted or polysubstituted. Moreover, there is no restriction on the substitution site, for example, it may be ortho-, para- or meta-substituted.

The term “heteroaryl” should be understood as a monovalent monocyclic, bicyclic or tricyclic aromatic ring system group containing 1 to 5 heteroatoms independently selected from N, O and S, preferably a “5- to 20-membered heteroaryl”. The term “5- to 20-membered heteroaryl” should be understood to include such a monovalent monocyclic, bicyclic or tricyclic aromatic ring system group which has 5 to 20 ring atoms and contain 1 to 5 heteroatoms independently selected from N, O and S, for example, a “5- to 14-membered heteroaryl”. The term “5- to 14-membered heteroaryl” should be understood to include such a monovalent monocyclic, bicyclic or tricyclic aromatic ring system group which has 5, 6, 7, 8, 9, 10, 11, 12, 13 or 14 ring atoms, in particular 5 or 6 or 9 or 10 carbon atoms, and which contains 1 to 5, preferably 1 to 3 heteroatoms each independently selected from N, O and S; and, in each case, it may additionally be benzo-fused. In particular, the heteroaryl is selected from the group consisting of thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl and the like and benzo derivatives thereof, such as benzofuranyl, benzothienyl, benzoxazolyl, benzisoxazolyl, benzimidazolyl, benzotriazolyl, indazolyl, indolyl, isoindolyl and the like; or pyridyl, pyridazinyl, pyrimidinyl, pyrazinyl, triazinyl and the like and benzo derivatives thereof, such as quinolyl, quinazolinyl, isoquinolyl and the like; or azocinyl, indolizinyl, purinyl and the like and benzo derivatives thereof; or cinnolinyl, phthalazinyl, quinazolinyl, quinoxalinyl, naphthyridinyl, pteridinyl, carbazolyl, acridinyl, phenazinyl, phenothiazinyl, phenoxazinyl and the like. When the 5- to 20-membered heteroaryl is connected to other groups to form the compounds disclosed herein, the carbon atoms on the 5- to 20-membered heteroaryl ring may be connected to other groups, or the heteroatoms on the 5- to 20-membered heteroaryl ring may be connected to other groups. When the 5- to 20-membered heteroaryl is substituted, it may be monosubstituted or polysubstituted. In addition, there is no limitation on the substitution site, for example, the hydrogen attached to the carbon atom on the heteroaryl ring may be substituted, or the hydrogen attached to the heteroatom on the heteroaryl ring may be substituted.

Unless otherwise specified, the heterocyclyl, heteroaryl or heteroarylene includes all possible isomeric forms thereof, such as positional isomers thereof. Therefore, for some illustrative non-limiting examples, it may comprise forms with substitution or binding to other group at one, two or more positions selected from its 1-, 2-, 3-, 4-, 5-, 6-, 7-, 8-, 9-, 10-, 11-, 12-positions, etc. (if present), including pyridin-2-yl, pyridin-2-yl, pyridin-3-yl, pyridin-3-yl, pyridin-4-yl and pyridin-4-yl; thienyl or thienyl include thien-2-yl, thien-2-yl, thien-3-yl and thien-3-yl; pyrazol-1-yl, pyrazol-3-yl, pyrazol-4-yl, pyrazol-5-yl.

The term “oxo” refers to that a carbon atom, nitrogen atom or sulfur atom in a substituent is oxidized to form an oxy substituent (═O). It should be understood that when a carbon atom is substituted with oxy, a carbonyl —C(═O)— is formed.

Unless otherwise specified, the definitions of terms herein also apply to groups containing the terms. For example, the definition of alkyl also applies to alkyl in alkyloxy or cycloalkylalkyl; the definition of cycloalkyl also applies to cycloalkyl in cycloalkyloxy or cycloalkylalkyl; for example, the definition of C₁₋₁₀ alkyl also applies to C₁₋₁₀ alkyl in C₁₋₁₀ alkyloxy or C₃₋₁₀ cycloalkyl-C₁₋₁₀ alkyl; the definition of C₃₋₁₀ cycloalkyl also applies to C₃₋₁₀ cycloalkyl in C₃₋₁₀ cycloalkyloxy or C₃₋₁₀ cycloalkyl-C₁₋₁₀ alkyl.

Those skilled in the art will appreciate that the compounds disclosed herein may exist in the form of various pharmaceutically acceptable salts. If these compounds have a basic center, they may form acid addition salts; if these compounds have an acidic center, they may form base addition salts; if these compounds contain both an acidic center (e.g., a carboxyl group) and a basic center (e.g., an amino group), they may also form inner salts.

The compounds of the present disclosure may exist in the form of solvates (e.g., hydrates), wherein the compounds of the present disclosure contain a polar solvent, in particular water, methanol or ethanol, as a structural element of crystal lattices of the compounds. The amount of polar solvent, in particular water, may be present in a stoichiometric or non-stoichiometric ratio.

According to their molecular structures, the compounds or groups of the present disclosure may be chiral or have chiral carbon atoms, and thus may exist in various enantiomeric forms. Thus, these compounds or groups may exist in racemic forms or optically active forms. For example, when the group A in the general Formula (G), (G′) or (GH) is C—R^{A}, and the groups to which the carbon atom is attached are not the same, the carbon atom is a chiral carbon atom, which may have a chiral configuration of R or S. The compounds or intermediates thereof of the present disclosure may be separated into enantiomeric compounds by chemical or physical methods known to those skilled in the art, or used in such form for synthesis. In the case of racemic amines, diastereomers may be prepared from the mixture by reaction with an optically active resolving agent. Examples of suitable resolving agents are optically active acids, such as R and S forms of tartaric acid, diacetyltartaric acid, dibenzoyltartaric acid, mandelic acid, malic acid, lactic acid, appropriate N-protected amino acids (e.g., N-benzoylproline or N-phenylsulfonylproline) or various optically active camphorsulfonic acids. Chromatographic enantiomeric resolution can also be advantageously performed with the aid of optically active resolving agents (e.g., dinitrobenzoylphenylglycine, cellulose triacetate or other carbohydrate derivatives or chirally derivatized methacrylate polymers immobilized on silica gel). Suitable eluents for this purpose are aqueous or alcoholic solvent mixtures, for example, hexane/isopropanol/acetonitrile.

The term “tautomer” refers to functional group isomers resulting from the rapid movement of an atom between two positions in a molecule. The compounds of the present disclosure may exhibit tautomerism. Tautomeric compounds may exist as two or more interconvertible species. Prototropic tautomers result from the migration of a covalently bonded hydrogen atom between two atoms. Tautomers generally exist in equilibrium, and attempts to separate a single tautomer generally result in a mixture whose physicochemical properties are consistent with the mixture of compounds. The position of the equilibrium depends on the intramolecular chemical properties. For example, in many aliphatic aldehydes and ketones such as acetaldehyde, the keto form predominates, while in phenols, the enol form predominates. The present disclosure encompasses all tautomeric forms of the compounds.

The corresponding stable isomers can be separated according to known methods, such as by extraction, filtration or column chromatography.

The term “patient” refers to any animal, including mammals, preferably mice, rats, other rodents, rabbits, dogs, cats, pigs, cattle, sheep, horses or primates, and most preferably humans.

The phrase “therapeutically effective amount” as used herein refers to the amount of active compound or drug that causes a biological or medical response that a researcher, veterinarian, physician or other clinician is seeking in a tissue, system, animal, individual or human, which comprises one or more of the following: (1) prevention of disease: for example, prevention of disease, disorder or condition in an individual who is susceptible to the disease, disorder or condition but has not yet experienced or developed the pathology or symptoms of the disease; (2) inhibition of disease: for example, inhibition of disease, disorder or condition in an individual who is experiencing or developing the pathology or symptoms of the disease, disorder or condition (i.e., preventing further development of the pathology and/or symptoms); (3) relief of disease: for example, relief of disease, disorder or condition in an individual who is experiencing or developing the pathology or symptoms of the disease, disorder or condition (i.e., reversing the pathology and/or symptoms).

The term “ligand” refers to a macromolecular compound that can recognize and bind to an antigen or receptor associated with a target cell. The role of ligand is to present a drug to a target cell population bound to the ligand, and the examples of ligand include but are not limited to protein hormone, lectin, growth factor, antibody or other molecules that can bind to cells. In the embodiment of the present disclosure, the ligand is represented by LG, and the ligand can form a connection bond with the connecting unit through a heteroatom on the ligand. The preferred ligand is an antibody or antigen-binding fragment thereof, and the antibody is selected from a chimeric antibody, a humanized antibody, a fully human antibody or a murine antibody; preferably a monoclonal antibody.

The term “drug” refers to any compound having a desired biological activity and a reactive functional group, and the reactive function group can be used to incorporate the drug into the conjugate of the present disclosure. The desired biological activity includes diagnosis, cure, relief, treatment, or prevention of diseases in humans or other animals. The reactive functional group forms a bond with the functional group L⁴ or L⁵. In some embodiments, the drug has a nitrogen atom or a hydroxyl group that can form a bond with the functional group L⁴ or L⁵.

The term “antibody” refers to an immunoglobulin, which comprises a tetrapeptide chain structure formed by two identical heavy chains and two identical light chains connected by interchain disulfide bonds (also known as “monoantibody” or “monoepitope antibody”), or a tetrapeptide chain structure formed by two different heavy chains and two different light chains connected by interchain disulfide bonds (also known as “bi-antibody” or “bi-epitope antibody”). Immunoglobulins are different in amino acid composition and arrangement order of heavy chain constant region, and thus are different in antigenicity. Based on this, immunoglobulins can be divided into five types, or called immunoglobulin isotypes, namely IgM, IgD, IgG, IgA and IgE, and their corresponding heavy chains are μ chain, δ chain, γ chain, α chain, and ε chain respectively. According to the difference in the amino acid composition of hinge region and the number and position of heavy chain disulfide bonds, the same type Ig can be divided into different subtypes, for example, IgG can be divided into IgG1, IgG2, IgG3, and IgG4. Light chains are divided into κ chain or λ chain according to the differences in constant region. Each of the five types of Ig can have κ chain or λ chain. The antibody described in the present disclosure is preferably a specific antibody against a cell surface antigen on a target cell, and its non-limiting examples are the following antibodies: one or more of anti-HER2 (ErbB2) antibody, anti-EGFR antibody, anti-B7-H3 antibody, anti-c-Met antibody, anti-HER3 (ErbB3) antibody, anti-HER4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody; preferably Trastuzumab (trade name Herceptin), Pertuzumab (also known as 2C4, trade name Perjeta), Nimotuzumab (trade name Taixinsheng), Enoblituzumab, Emibetuzumab, Inotuzumab, Pinatuzumab, Brentuximab, Gemtuzumab, Bivatuzumab, Lorvotuzumab, cBR96, and Glematumamab or Glembatumumab.

The sequences of about 110 amino acids near the N-terminals of antibody heavy chain and light chain vary greatly, which are variable regions (Fv regions); the remaining amino acid sequences near the C-terminals are relatively stable, which are constant regions. The variable regions include three hypervariable regions (HVR) and four relatively conservative framework regions (FR). The three hypervariable regions determine the specificity of antibody, also known as complementarity determining region (CDR). Each of light chain variable region (LCVR) and heavy chain variable region (HCVR) consists of three CDR regions and four FR regions, arranged in the following order from the amino terminus to the carboxyl terminus: FR1, CDR1, FR2, CDR2, FR3, CDR3, FR4. The three CDR regions of light chain refer to LCDR1, LCDR2, and LCDR3; the three CDR regions of heavy chain refer to HCDR1, HCDR2, and HCDR3.

The antibodies disclosed herein comprise murine antibody, chimeric antibody, humanized antibody, and fully human antibody, preferably humanized antibody and fully human antibody.

The term “murine antibody” in the present disclosure refers to an antibody prepared from mice according to the knowledge and skills in the art. During preparation, the test subject is injected with a specific antigen, and then a hybridoma expressing an antibody with the desired sequence or functional properties is isolated.

The term “chimeric antibody” refers to an antibody formed by fusing the variable region of a murine antibody with the constant region of a human antibody, which can reduce the immune response induced by the murine antibody. To establish a chimeric antibody, it is necessary to first establish a hybridoma that secretes a mouse-specific monoclonal antibody, then the variable region gene is cloned from the mouse hybridoma cells, and then the constant region gene of human antibody is cloned if necessary, the mouse variable region gene is ligated to the human constant region gene to form a chimeric gene, which is then inserted into an expression vector, and finally the chimeric antibody molecule is expressed in an eukaryotic system or a prokaryotic system.

The term “humanized antibody”, also known as CDR-grafted antibody, refers to an antibody produced by grafting a mouse CDR sequence into a human antibody variable region framework, that is, a human germline antibody framework sequence of different type. It can overcome the heterologous response induced by chimeric antibodies due to carrying a large amount of mouse protein components. Such framework sequences can be obtained from public DNA databases or public references that include germline antibody gene sequences. For example, germline DNA sequences of human heavy and light chain variable region genes can be found in the “VBase” human germline sequence database (available on the Internet at www.mrccpe.com.ac.uk/vbase), and in Kabat, E. A. et al., 1991 Sequences of Proteins of Immunological Interest, 5th edition. In order to avoid a decrease in activity caused by a decrease in immunogenicity, minimal reverse mutations or back mutations may be performed on the human antibody variable region framework sequences to maintain activity. The humanized antibodies disclosed herein also comprise humanized antibodies after CDR affinity maturation has been further performed by phage display. Literature further describing methods for humanizing murine antibodies includes, for example, Queen et al., Proc., Natl. Acad. Sci. USA, 88, 2869, 1991, and the methods of Winter and his colleagues [Jones et al., Nature, 321, 522 (1986), Riechmann, et al., Nature, 332, 323-327 (1988), Verhoeyen, et al., Science, 239, 1534 (1988)].

The term “fully human antibody”, “fully humanized antibody” or “completely human antibody” is also called “fully human monoclonal antibody”, in which both the variable region and the constant region of antibody are humanized, thereby eliminating immunogenicity and toxic side effects. The development of monoclonal antibodies has gone through four stages, namely: murine monoclonal antibodies, chimeric monoclonal antibodies, humanized monoclonal antibodies and fully human monoclonal antibodies. The present disclosure involves a fully human monoclonal antibody. The main technologies related to the preparation of fully human antibodies include: human hybridoma technology, EBV-transformed B lymphocyte technology, phage display technology, transgenic mouse antibody preparation technology, and single B cell antibody preparation technology.

The term “antigen-binding fragment” refers to one or more fragments of an antibody that retain the ability to specifically bind to an antigen. It has been shown that fragments of a full-length antibody can be used to perform the antigen-binding function of antibody. Examples of binding fragments included in “antigen binding fragments” include: (i) Fab fragment, a monovalent fragment consisting of VL, VH, CL and CH₁ domains; (ii) F(ab′)₂ fragment, a bivalent fragment comprising two Fab fragments connected by a disulfide bridge at the hinge region, (iii) Fd fragment, consisting of VH and CH1 domains; (iv) Fv fragment, consisting of the VH and VL domains of a single arm of an antibody; (v) single domain or dAb fragment (Ward et al., (1989) Nature 341: 544-546), consisting of a VH domain; and (vi) isolated complementarity determining region (CDR); or (vii) a combination of two or more isolated CDRs, optionally connected by a synthetic linker. In addition, although the two domains VL and VH of Fv fragment are encoded by separate genes, they can be connected by a synthetic linker using recombinant methods, so that they can be produced as a single protein chain in which the VL and VH regions are paired to form a monovalent molecule (called single-chain Fv (scFv); see, for example, Bird et al. (1988) Science 242: 423-426; and Huston et al. (1988) Proc. Natl. Acad. Sci USA 85: 5879-5883). Such single-chain antibodies are also intended to be included in the term “antigen-binding fragment” of antibody. Such antibody fragments are obtained using conventional techniques known to those skilled in the art, and the fragments are screened for functionality in the same manner as for intact antibodies. Antigen-binding fragments can be produced by recombinant DNA technology or by enzymatic or chemical cleavage of intact immunoglobulins. The antibody may be an antibody of different isotype, for example, an IgG (e.g., IgG1, IgG2, IgG3 or IgG4 subtype), IgAQ1, IgA2, IgD, IgE or IgM antibody.

Fab is an antibody fragment having a molecular weight of about 50,000 and having antigen-binding activity among fragments obtained by treating an IgG antibody molecule with protease papain (cleaving the amino acid residue at position 224 of H chain), wherein about half of the N-terminal side of the H chain and the entire L chain are bound together by disulfide bonds.

F(ab′)2 is an antibody fragment having a molecular weight of about 100,000 and having antigen-binding activity, which is obtained by digesting the lower part of two disulfide bonds in the hinge region of IgG with the enzyme pepsin and comprises two Fab regions connected at the hinge position.

Fab′ is an antibody fragment having a molecular weight of about 50,000 and having antigen-binding activity, which is obtained by cleaving the disulfide bonds of hinge region of the above-mentioned F(ab′)2.

In addition, the Fab′ can be produced by inserting the DNA encoding the Fab′ fragment of the antibody into a prokaryotic expression vector or an eukaryotic expression vector and introducing the vector into a prokaryotic organism or an eukaryotic organism to express the Fab′.

The term “single-chain antibody”, “single-chain Fv” or “scFv” refers to a molecule comprising an antibody heavy chain variable domain (or region; VH) and an antibody light chain variable domain (or region; VL) connected by a linker. Such scFv molecules may have a general structure: NH₂-VL-linker-VH—COOH or NH₂—VH-linker-VL-COOH. Suitable prior art linkers consist of repeated GGGGS amino acid sequences or variants thereof, for example, variants using 1 to 4 repeats (Holliger et al. (1993), Proc. Natl. Acad. Sci. USA 90: 6444-6448). Other linkers that can be used in the present disclosure are described by Alfthan et al. (1995), Proc. Natl. Acad. Sci. USA 90: 6444-6448.otein Eng.8:725-731, Choi et al. (2001), Eur. J. Immunol.31:94-106, Hu et al. (1996), Cancer Res.56:3055-3061, Kipriyanov et al. (1999), J. Mol. Biol.293:41-56, and Roovers et al. (2001), Cancer Immunol.

The term “CDR” refers to one of the six hypervariable regions within the variable domain of an antibody that primarily contribute to antigen binding. One of the most commonly used definitions of the six CDRs is provided by Kabat E. A. et al. (1991) Sequences of proteins of immunological interest. NIH Publication 91-3242). As used herein, the Kabat definition of CDR applies only to CDR1, CDR2 and CDR3 (CDR L1, CDR L², CDR L3 or L1, L2, L3) of the light chain variable domain, and CDR2 and CDR3 (CDR H2, CDR H3 or H2, H3) of the heavy chain variable domain.

The term “antibody framework” refers to a portion of variable domain VL or VH that serves as a scaffold for antigen-binding loop (CDR) of that variable domain. Essentially, it is a variable domain without CDR.

The term “epitope” or “antigenic determinant” refers to a site on an antigen to which an immunoglobulin or antibody specifically binds. An epitope typically comprises at least 3, 4, 5, 6, 7, 8, 9, 10, 11, 12, 13, 14 or 15 consecutive or non-consecutive amino acids in a unique spatial conformation. See, for example, Epitope Mapping Protocols in Methods in Molecular Biology, Vol. 66, G. E. Morris, Ed. (1996).

The terms “specific binding”, “selective binding”, “selectively binding” and “specifically binding” refer to the binding of an antibody to an epitope on a predetermined antigen. Typically, the antibody binds with an affinity (KD) of about less than 10⁻⁷M, for example, about less than 10⁻⁸M, 10⁻⁹M or 10⁻¹M or less.

The term “nucleic acid molecule” refers to DNA molecule and RNA molecule. Nucleic acid molecules can be single-stranded or double-stranded, but are preferably double-stranded DNA. A nucleic acid is “operably linked” when it is placed in a functional relationship with another nucleic acid sequence. For example, if a promoter or enhancer affects the transcription of a coding sequence, then the promoter or enhancer is operably linked to the coding sequence.

The term “vector” refers to a nucleic acid molecule capable of transporting another nucleic acid to which it has been ligated. In one embodiment, the vector is a “plasmid”, which refers to a circular double-stranded DNA loop into which an additional DNA segment can be ligated. In another embodiment, the vector is a viral vector, in which an additional DNA segment can be ligated to the viral genome. The vectors disclosed herein are capable of autonomous replication in a host cell into which they have been introduced (e.g., bacterial vectors with bacterial replication origins, and episomal mammalian vectors) or can be integrated into the genome of a host cell after introduction into the host cell, thereby replicating along with the host genome (e.g., non-episodic mammalian vectors).

Methods for producing and purifying antibodies and antigen-binding fragments are well known in the prior art, such as Guide to Antibody Experimental Technology, Chapters 5-8 and 15, the Cold Spring Harbor. Antigen-binding fragments can also be prepared by conventional methods. In the antibodies or antigen-binding fragments described in the present invention, one or more human FR regions are added into a non-human CDR region by using genetic engineering methods. Human FR germline sequences can be obtained from the ImMunoGeneTics (IMGT) website http://imgt.cines.fr by alignment on the IMGT human antibody variable region germline gene database and MOE software, or from the Journal of Immunoglobulins, 2001 ISBN012441351.

The term “host cell” refers to a cell into which an expression vector has been introduced. Host cells may include bacterial, microbial, plant or animal cells. Easily transformed bacteria include members of the enterobacteriaceae family, such as strains of Escherichia coli or Salmonella; Bacillaceae, such as Bacillus subtilis; Pneumococcus; Streptococcus and Haemophilus influenzae. Suitable microorganisms include Saccharomyces cerevisiae and Pichia pastoris. Suitable animal host cell lines include CHO (Chinese hamster ovary cell line) and NS0 cells.

The engineered antibodies or antigen-binding fragments disclosed herein can be prepared and purified by conventional methods. For example, cDNA sequences encoding heavy and light chains can be cloned and recombined into GS expression vectors. The recombinant immunoglobulin expression vector can stably transfect CHO cells. As a more recommended prior art, mammalian expression systems lead to glycosylation of antibodies, especially at the highly conserved N-terminal site in the Fc region. Positive clones are expanded in serum-free medium in a bioreactor to produce antibodies. The culture fluid that secretes antibodies can be purified by conventional techniques. For example, purification is performed using an A or G Sepharose FF column containing an adjusted buffer. Non-specifically bound components are washed away. The bound antibodies are then eluted using a pH gradient method, and the antibody fragments are detected by SDS-PAGE and collected. The antibodies can be filtered and concentrated by conventional methods. Soluble mixtures and polymers can also be removed by conventional methods, such as molecular sieves and ion exchange. The obtained product should be immediately frozen at, for example, −70° C., or freeze-dried.

The term “peptide” refers to a compound fragment between amino acids and proteins, which is composed of two or more amino acid molecules connected to each other by peptide bonds, and is a structural and functional fragment of protein. For example, hormones, enzymes and the like all are essentially peptides.

The term “sugar” refers to a biological macromolecule composed of three elements: C, H, and 0, which can be classified into monosaccharide, disaccharide, and polysaccharide.

### Beneficial Effects

The compounds and conjugates disclosed in the present invention have excellent tumor cell inhibition activity, stability, and in vivo efficacy in animals, and can be used as effective drugs for inhibiting tumor cells and preventing and/or treating cancer.

### BRIEF DESCRIPTION OF THE DRAWINGS

FIG. 1 shows the test results of the bystander killing effect of the antibody-drug conjugate of the present invention on tumor cells in Test Example 4;

FIG. 2 shows the test results of the growth inhibition on nude mouse NCI-N87 transplanted tumor in the experiment of Test 5A in Test Example 5;

FIG. 3 shows the summary results of the weight change of experimental mice in the experiment of Test 5A in Test Example 5;

FIG. 4 shows the test results of the growth inhibition of nude mouse NCI-N87 transplanted tumor in the experiment of Test 5B in Test Example 5;

FIG. 5 shows the summary results of the weight change of experimental mice in the experiment of Test 5B in Test Example 5;

FIG. 6 shows the test results of the dissociation degree value of the plasma stability of the antibody-drug conjugate of the present invention in Test Example 6;

FIG. 7 shows the test results of the growth inhibition on nude mouse NCI-N87 transplanted tumor in the experiment of Test 5C in Test Example 5;

FIG. 8 shows the summary results of the weight change of experimental mice in the experiment of Test 5C in Test Example 5;

FIG. 9 shows the results of the bystander killing test of Pertuzumab antibody-drug conjugate in Test Example 7;

FIG. 10 shows the results of the bystander killing test of antibody-drug conjugate on EGFR target tumor cells in Test Example 9;

FIG. 11 shows the test results of the growth inhibition on JIMT-1 mouse subcutaneous transplanted tumor model in Test Example 10;

FIG. 12 shows the summary results of the weight change of experimental mice in Test Example 10;

FIG. 13 shows the test results of the growth inhibition on NCI-H322 mouse subcutaneous transplanted tumor model in Test Example 11;

FIG. 14 shows the summary results of the weight change of experimental mice in Test Example 11;

FIG. 15 shows the test results of the bystander killing effect of ADC-113 on HER2 target tumor cells in Test Example 14;

FIG. 16 shows the test results of the bystander killing effect of ADC-115 on HER2 target tumor cells in Test Example 14;

FIG. 17 shows the test result of the growth inhibition on nude mouse NCI-N87 transplanted tumors administrated with different doses of ADC-113 in Test Example 16;

FIG. 18 shows the summary results of the weight change of experimental mice administrated with different doses of ADC-113 in Test Example 16;

FIG. 19 shows the test results of the growth inhibition on nude mouse NCI-N87 transplanted tumor administrated with ADC-115 in Test Example 17;

FIG. 20 shows the summary results of the weight change of experimental mice administrated with ADC-115 in Test Example 17.

### DETAILED DESCRIPTION

The technical solution of the present disclosure will be further described in detail below in conjunction with specific examples. It should be understood that the following embodiments are only exemplary illustrations and explanations of the present disclosure, and should not be interpreted as limiting the scope of protection of the present disclosure. All technologies realized based on the above contents of the present disclosure fall within the scope sought to be protected by the present disclosure.

Unless otherwise specified, the starting materials and reagents used in the following examples were commercially available products or could be prepared by known methods.

### I. Antibody Examples

The following antibodies were prepared according to conventional antibody methods, for example, after vector construction, eukaryotic cells such as HEK293 cells (Life Technologies Cat. No. 11625019) could be transfected.

The exemplary antibody sequences are as follows:

The following shows the sequences of Trastuzumab

light chain
SEQ ID NO: 1
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO: 2
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK

The following shows the sequences of Pertuzumab

light chain
SEQ ID NO.3
DIQMTQSPSSLSASVGDRVTITCKASQDVSIGVAWYQQKPGKAPK
LLIYSASYRYTGVPSRFSGSGSGTDFTLTISSLQPEDFATYYCQQ
YYIYPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO. 4
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK

The following shows the sequences of Nimotuzumab

light chain
SEQ ID NO: 5
DIQMTQSPSSLSASVGDRVTITCRSSQNIVHSNGNTYLDWYQQTP
GKAPKLLIYKVSNRFSGVPSRFSGSGSGTDFTFTISSLQPEDIAT
YYCFQYSHVPWTFGQGTKLQITREVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
heavy chain
SEQ ID NO: 6
QVQLQQSGAEVKKPGSSVKVSCKASGYTFTNYYIYWVRQAPGQGL
EWIGGINPTSGGSNFNEKFKTRVTITVDESTNTAYMELSSLRSED
TAFYFCARQGLWFDSDGRGFDFWGQGSTVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK

The following shows the sequences of Patritumab

light chain
SEQ ID NO: 7
DIEMTQSPDSLAVSLGERATINCRSSQSVLYSSSNRNYLAWYQQN
PGQPPKLLIYWASTRESGVPDRFSGSGSGTDFTLTISSLQAEDVA
VYYCQQYYSTPRTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
heavy chain
SEQ ID NO: 8
QVQLQQWGAGLLKPSETLSLTCAVYGGSFSGYYWSWIRQPPGKGL
EWIGEINHSGSTNYNPSLKSRVTISVETSKNQFSLKLSSVTAADT
AVYYCARDKWTWYFDLWGRGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKRVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK

### II. Compound Examples

The structures of the compounds were determined by nuclear magnetic resonance (NMR) or mass spectrometry (MS). Chemical shifts 6 are given in units of 10⁻⁶ (ppm). NMR measurements were performed by using a Bruker nuclear magnetic spectrometer. The solvents used were deuterated dimethyl sulfoxide (DMSO-d6), deuterated chloroform (CDCl₃), and deuterated methanol (CD₃OD). The internal standard was tetramethylsilane (TMS).

LCMS was performed by using: Agilent 1260 Infinity II (ESI) mass spectrometer, Waters UPLC H Class plus (ESI) or Shimadzu LCMS-2020 (ESI).

High performance liquid chromatography (HPLC) analysis was performed by using: Agilent 1260 or Shimadzu LC-20AD.

Preparative high performance liquid chromatography (pre-HPLC) was performed by using: GILSON GX-281 or Agilent 1260 Infinity II preparative HPLC.

Chiral preparation was performed by supercritical fluid chromatography (SFC) using Shimadzu LC-30Adsf or Shimadzu LC-20AD.

Thin layer chromatography silica gel plate used was GF254 acrylic adhesive silica gel plate produced by Anhui Liangchen Silicon Source Material Co., Ltd. The silica gel plate used in thin layer chromatography (TLC) was 0.2 mm silica gel plate, and 0.5 mm silica gel plate was used for separation and purification of product by thin layer chromatography.

For column chromatography, 200 to 300 mesh silica gel from Anhui Liangchen Silicon Source Materials Co., Ltd. was generally used as carrier.

Kinase average inhibition rate and IC₅₀ value were determined using SpectraMax i3X microplate reader (MD Company, USA).

The known starting materials of the present disclosure could be synthesized by methods known in the art, or could be purchased from companies such as Bitec Pharmaceuticals, Leyan, Shaoyuan Chemical Technology, and Annaiji Chemicals.

In the following examples, unless otherwise specified, the reactions were carried out under argon or nitrogen atmosphere.

Argon or nitrogen atmosphere meant that the reaction bottle was connected to an argon or nitrogen balloon with a volume of about 1 L.

Hydrogen atmosphere meant that the reaction bottle was connected to a hydrogen balloon with a volume of about 1 L.

Hydrogenation reaction was usually carried out by repeating three times of the operations of vacuuming and filling hydrogen.

Oxygen atmosphere meant that the reaction bottle was connected to an oxygen balloon with a volume of about 1 L.

In the following examples, unless otherwise specified, the solution referred to an aqueous solution, and the reaction temperature was room temperature, which was 20° C. to 30° C.

The progress of the reactions in the examples was monitored by thin layer chromatography (TLC) using developing solvents, the eluent system of column chromatography used for purifying compounds and the developing solvent system of thin layer chromatography included: A: dichloromethane/methanol system, B: petroleum ether/ethyl acetate system, in which the solvent volume ratio was adjusted according to the polarity of compounds, and a small amount of alkaline or acidic reagent such as triethylamine and acetic acid could also be added for adjustment.

### Example 2-1: Preparation of Compound 1 and its Isomers 1-A, 1-B, 1-C and 1-D

### N-((1R,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 1-A

### N-((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 1-B

### N-((1S,9R)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 1-C

### N-((1R,9R)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 1-D

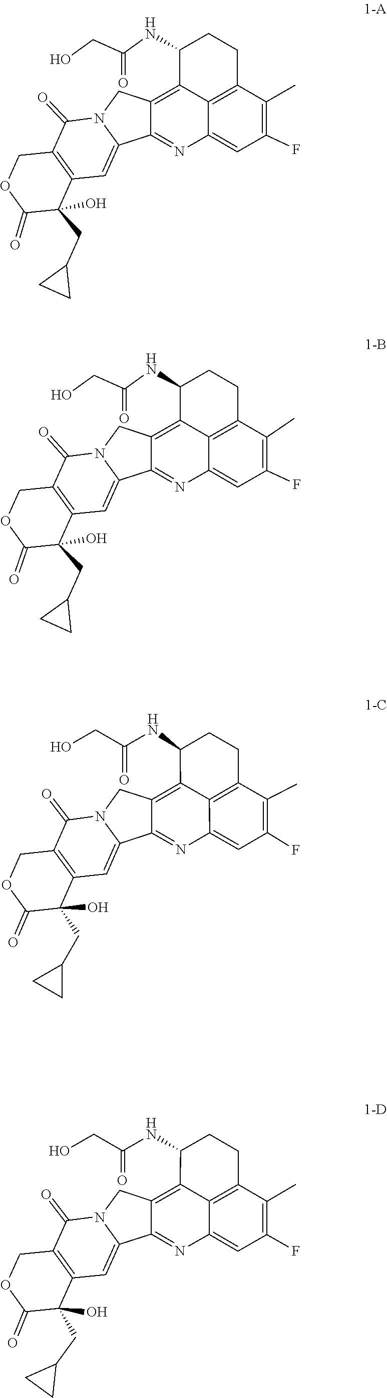 [SMILES: Cc1c(F)cc2nc3c(c4c2c1CC[C@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@@]2(O)CC1CC1 ; Cc1c(F)cc2nc3c(c4c2c1CC[C@H]4NC(=O)CO)Cn1c-3cc2c(c1=O)COC(=O)[C@@]2(O)CC1CC1]

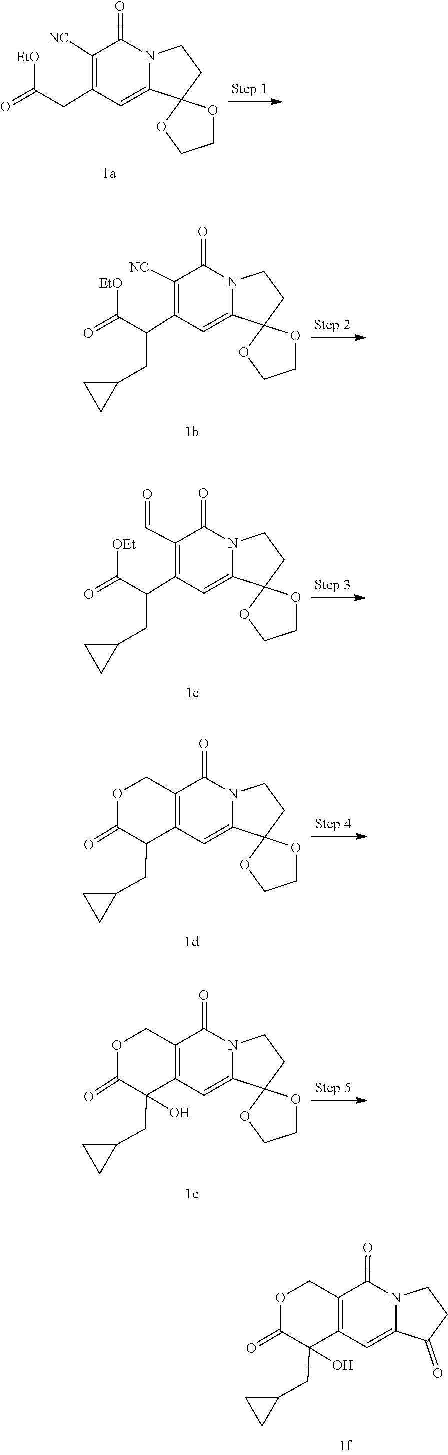 [SMILES: CCOC(=O)Cc1cc2n(c(=O)c1C#N)CCC21OCCO1 ; CCOC(=O)C(CC1CC1)c1cc2n(c(=O)c1C#N)CCC21OCCO1 ; CCOC(=O)C(CC1CC1)c1cc2n(c(=O)c1C=O)CCC21OCCO1 ; O=C1OCc2c(cc3n(c2=O)CCC32OCCO2)C1CC1CC1 ; O=C1OCc2c(cc3n(c2=O)CCC32OCCO2)C1(O)CC1CC1 ; O=C1CCn2c1cc1c(c2=O)COC(=O)C1(O)CC1CC1]

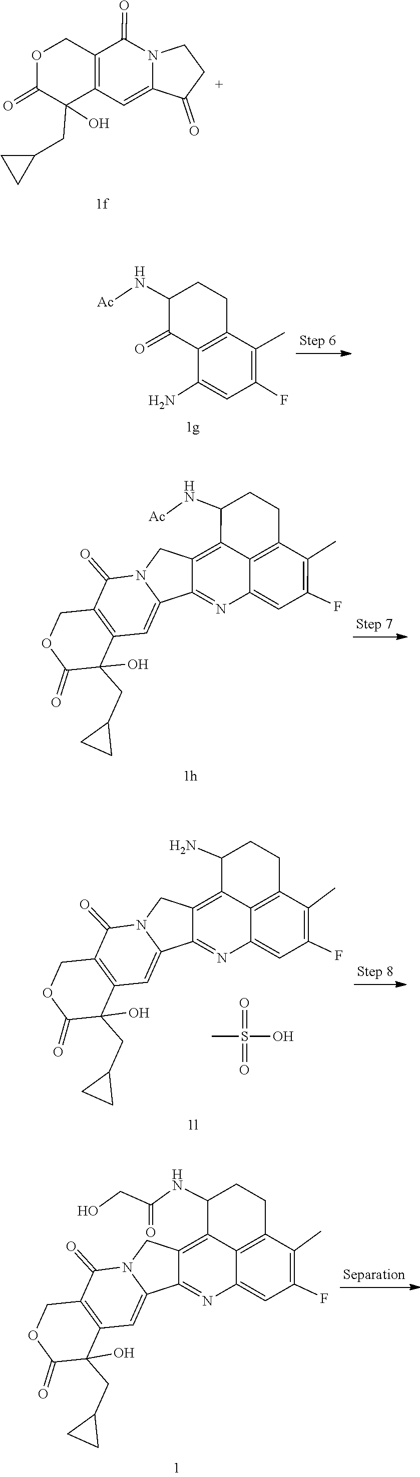

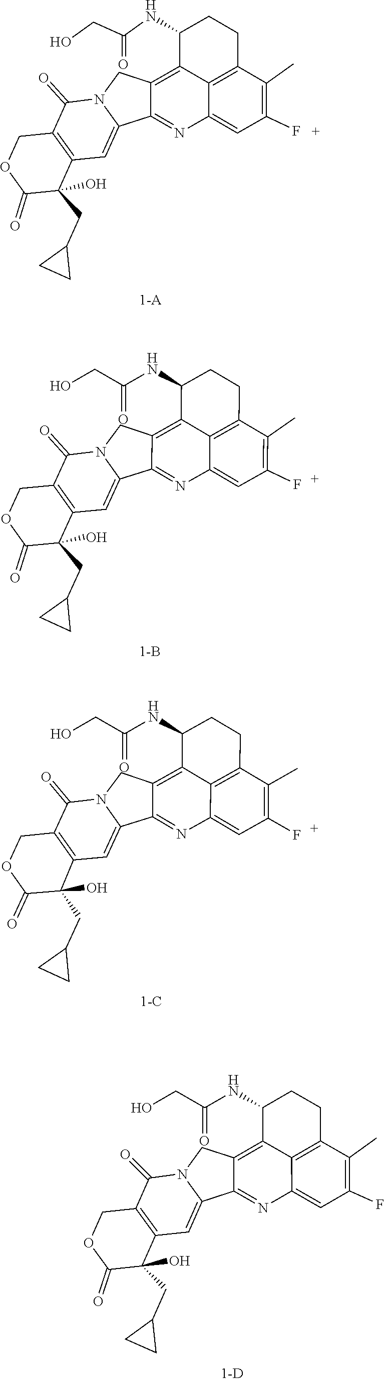 [SMILES: Cc1c(F)cc2nc3c(c4c2c1CC[C@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@@]2(O)CC1CC1 ; Cc1c(F)cc2nc3c(c4c2c1CC[C@H]4NC(=O)CO)Cn1c-3cc2c(c1=O)COC(=O)[C@@]2(O)CC1CC1]

### Step 1

### Ethyl 2-(6-cyano-5-oxo-2,3-dihydro-5H-spiro[indolizine-1,2′-[1,3]dioxolan]-7-yl)-3-cyclopropyl-propanoate 1b

1a (1.01 g, 3.31 mmol, prepared by applying the method disclosed in Example 30 on page 28 of patent application “WO2019238046”) was dissolved in 15 mL of acetonitrile, added with bromomethyl cyclopropane (894.93 mg, 6.63 mmol) and potassium carbonate (916.16 mg, 6.63 mmol), and stirred at 80° C. for 13 hours. 10 mL of water was added. The diluted reaction solution was extracted with ethyl acetate (15 mL×2). The organic phase was washed with saturated sodium chloride solution (10 mL×2), then dried over anhydrous sodium sulfate and filtered, and the filtrate was concentrated by vacuum distillation. The resulting residue was purified by silica gel column chromatography using the eluent system A to obtain the title product 1b (1.12 g, yield: 92%) in the form of yellow solid.

MS m/z (ESI): 359.1 [M+1].

### Step 2

### Ethyl 3-cyclopropyl-2-(6-formyl-5-oxo-2,3-dihydro-5H-spiro[indolizine-1,2′-[1,3]dioxolan]-7-yl)propionate 1c

1b (1.12 g, 3.05 mmol) was dissolved in a mixed solvent of 5 mL water, 5 mL acetonitrile and 5 mL formic acid, added with Raney nickel (261.72 mg) and subjected to replacement with hydrogen gas for three times. The reaction mixture was stirred at 60° C. for 4 hours under hydrogen gas (15 Psi). The reaction mixture was filtered through Celite. The filter cake was washed with dichloromethane (50 mL×3). The filtrate was washed with aqueous hydrochloric acid (4M, 20 mL), and then washed with sodium carbonate aqueous solution (12M, 50 mL). The organic phase was dried over anhydrous sodium sulfate and filtered. The filtrate was concentrated by vacuum distillation. The residue was purified by reverse phase liquid chromatography (separation conditions: chromatographic column: 120 g Flash Column Welch Ultimate XB_C18 20-40 μm; mobile phase: A-water: B-acetonitrile, gradient elution, flow rate: 85 mL/min, instrument: ISCO) to obtain the title product 1c (680 mg, yield: 60%) in the form of yellow solid.

MS m/z (ESI): 362.1 [M+1].

### Step 3

### 4-(Cyclopropylmethyl)-1,4,7,8-tetrahydro-3H,10H-spiro[pyrano[3,4-f]indolizine-6,2′-[1,3]dioxolane]-3,10-dione 1d

1c (680 mg, 1.85 mmol) was dissolved in 10 mL of dichloromethane under protection of nitrogen gas, cooled to 0° C. in ice-water bath, and added with sodium borohydride (108.02 mg, 2.86 mmol) in batches, and the reaction solution was stirred at 0° C. for 30 minutes. At 25° C., acetic acid (133.15 mg, 2.22 mmol) was added dropwise, gas was generated, and stirring was continued for 2 hours. At 15° C., 30 mL of water was added dropwise, gas was generated, and stirring was continued at 15° C. for 1.5 hours. The reaction solution was washed with water (50 mL), the washed organic phase was added with p-toluenesulfonic acid monohydrate (35.15 mg, 184.78 μmol) at 15° C. and stirred at 15° C. for 12 hours. 50 mL of water was added and the reaction solution was extracted with dichloromethane (45 mL×3), the organic phase was dried over anhydrous sodium sulfate and filtered, and the filtrate was concentrated by vacuum distillation. The obtained residue was purified by silica gel column chromatography using the developing system B to obtain the title product 1d (200 mg, yield: 32%) in the form of yellow oil.

MS m/z (ESI): 318.1 [M+1].

### Step 4

### 4-(Cyclopropylmethyl)-4-hydroxy-1,4,7,8-tetrahydro-3H,10H-spiro[pyrano[3,4-f]indolizine-6,2′-[1,3]dioxolane]-3,10-dione 1e

1d (202.13 mg, 598.74 μmol) was dissolved in 0.5 ml of methanol, cooled to 0° C. in ice-water bath, added with potassium carbonate (82.75 mg, 598.74 μmol) in batches, and stirred at 0° C. for 5 hours under oxygen bubbling (15 psi). The reaction solution was poured into 10 mL of saturated ammonium chloride aqueous solution, and concentrated by vacuum distillation to remove methanol. The reaction solution was extracted with dichloromethane (20 mL×3). The organic phase was dried over anhydrous sodium sulfate and filtered, and concentrated by vacuum distillation to obtain the crude title product 1e (140 mg) in the form of yellow oil, and the product was used directly in the next step without purification.

MS m/z (ESI): 334.1 [M+1].

### Step 5

### 4-(Cyclopropylmethyl)-4-hydroxy-7,8-dihydro-1H-pyrano[3,4-]indolizine-3,6,10(4H)-trione 1f

1e (64.52 mg, 180.00 μmol) was dissolved in a mixed solution of 0.5 mL of trifluoroacetic acid and 0.125 mL of water, and stirred at 25° C. for 1 hour. The reaction solution was concentrated by vacuum distillation, and the resultant residue without purification was used to obtain the crude title product if (40 mg) in the form of yellow solid, and the product was used directly in the next reaction without purification.

MS m/z (ESI): 290.1 [M+1].

### Step 6 N-(9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)acetamide 1h

1f (40 mg, 128.59 μmol) and 1 g (38.62 mg, 154.31 μmol, prepared by applying the method disclosed in Example 5-1 on page 61 of patent application “CN11065621A”), pyridinium 4-toluene sulfonate (6.46 mg, 25.72 μmol) was dissolved in 0.5 mL of toluene, and stirred at 130° C. for 2 hours under nitrogen protection. The reaction solution was filtered through Celite, the filtrate was added with 10 mL of water and extracted with dichloromethane (8 ml×3). The organic phase was dried over anhydrous sodium sulfate and filtered, and the filtrate was concentrated under reduced pressure. The resulting residue was purified by preparative thin-layer chromatography using the eluent system A to obtain the title product 1h (15 mg, yield: 23%) in the form of brown solid.

MS m/z (ESI): 504.2 [M+1].

### Step 7

### 1-Amino-9-(cyclopropylmethyl)-5-fluoro-9-hydroxy-4-methyl-1,2,3,9,12,15-hexahydro-10H,13H-benzo[de]pyrano[3′,4′:6,7]indolizino[1,2-b]quinoline-10,13-dione methanesulfonate 1i

1h (50 mg, 96.5 μmol) was dissolved in 1 mL of 1,2-dimethoxyethane, added with 0.5 mL of methanesulfonic acid and 0.5 mL of water, and stirred at 85° C. for 18 hours. The reaction solution was poured into 10 mL o

f water, adjusted to pH 7-8 with saturated sodium bicarbonate solution, and extracted with dichloromethane (10 mL×5). The organic phase was dried over anhydrous sodium sulfate and filtered, and the filtrate was concentrated under reduced pressure to obtain the crude title product 1i (40 mg) in the form of brown solid. The product was used directly in the next reaction without purification.

MS m/z (ESI): 462.3 [M+1].

### Step 8

N-((1R,9S)-9-(Cyclopropylmethyl)-5-fluoro-9-hydroxy-4-methyl-10,13-dioxo-2,3,9,10,13, 15-hexahydro-1H,12H-benzo[de]pyrano[3′,4′:6,7]indolizino[1,2-b]quinolin-1-yl)-2-hydroxyacetamide 1-A
N-((1S,9S)-9-(Cyclopropylmethyl)-5-fluoro-9-hydroxy-4-methyl-10,13-dioxo-2,3,9,10,13, 15-hexahydro-1H,12H-benzo[de]pyrano[3′,4′:6,7]indolizino[1,2-b]quinolin-1-yl)-2-hydroxyacetamide 1-B
N-((1S,9R)-9-(Cyclopropylmethyl)-5-fluoro-9-hydroxy-4-methyl-10,13-dioxo-2,3,9,10,13, 15-hexahydro-1H,12H-benzo[de]pyrano[3′,4′:6,7]indolizino[1,2-b]quinolin-1-yl)-2-hydroxyacetamide 1-C
N-((1R,9R)-9-(Cyclopropylmethyl)-5-fluoro-9-hydroxy-4-methyl-10,13-dioxo-2,3,9,10,13, 15-hexahydro-1H,12H-benzo[de]pyrano[3′,4′:6,7]indolizino[1,2-b]quinolin-1-yl)-2-hydroxyacetamide 1-D

The mesylate of 1i (35 mg, 62.2 μmol) and glycolic acid (5.7 mg, 74.6 μmol) were dissolved in 1.5 mL of N,N-dimethylformamide, then added with 1-hydroxybenzotriazole (12.6 mg, 93.2 μmol), N,N-diisopropylethylamine (16.1 mg, 124 μmol) and 1-(3-dimethylaminopropyl)-3-ethylcarbodiimide hydrochloride (17.9 mg, 93.2 μmol) in sequence, and stirred at 25° C. for 1 hour. The reaction solution was added with 20 mL of water, and the diluted reaction solution was extracted with dichloromethane (10 mL×5). The organic phase was dried over anhydrous sodium sulfate and filtered, and the filtrate was concentrated under reduced pressure. The residue was purified by supercritical fluid chromatography to obtain four single-configuration title products 1-A, 1-B, 1-C and 1-D:

### Single-Configuration Compound I of Compound 1 (Designated as 1-B, 1.02 mg)

SFC analysis: retention time: 1.472 minutes, purity: 98%. (Chromatographic column: Chiralpak IC-3 50×4.6 mm I.D., 3 μm, mobile phase: A-carbon dioxide, B-methanol and acetonitrile (0.05% diethylamine), isocratic elution: B: 60%, flow rate: 3 mL/min, instrument: Shimadzu LC-30ADsf).

MS m/z (ESI): 520.5 [M+1].

¹H NMR (400 MHz, CD₃OD) δ 7.71-7.61 (m, 2H), 5.73-5.66 (m, 1H), 5.63-5.56 (m, 1H), 5.40-5.32 (m, 2H), 5.12-5.06 (m, 1H), 4.28-4.22 (m, 1H), 4.18-4.12 (m, 1H), 3.32-3.32 (m, 1H), 3.20-3.11 (m, 1H), 2.42 (s, 3H), 2.39-2.30 (m, 1H), 2.29-2.19 (m, 1H), 1.97-1.89 (m, 1H), 1.88-1.80 (m, 1H), 0.94-0.86 (m, 1H), 0.51-0.38 (m, 2H), 0.14-0.07 (m, 1H), 0.04-−0.01 (m, 1H).

### Single-Configuration Compound II of Compound 1 (Designated as 1-A, 1.02 mg)

SFC analysis: retention time: 2.075 minutes, purity: 93%. (Chromatographic column: Chiralpak AS-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 520.4 [M+1].

¹H NMR (400 MHz, CD₃OD) δ 7.66-7.53 (m, 2H), 5.70-5.63 (m, 1H), 5.60-5.54 (m, 1H), 5.41-5.31 (m, 2H), 5.22-5.14 (m, 1H), 4.34-4.23 (m, 1H), 4.22-4.11 (m, 1H), 3.43-3.36 (m, 1H), 3.22-3.13 (m, 1H), 2.43-2.37 (m, 3H), 2.35-2.26 (m, 2H), 1.95-1.80 (m, 2H), 0.93-0.86 (m, 1H), 0.55-0.35 (m, 2H), 0.17-0.08 (m, 1H), 0.07-−0.03 (m, 1H).

### Single-Configuration Compound III of Compound 1 (1.21 mg)

SFC analysis: retention time: 0.468 minutes, purity: 98%. (Chromatographic column: Chiralcel OD-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 520.1 [M+1].

¹H NMR (400 MHz, CD₃OD) δ 7.71-7.64 (m, 2H), 5.73-5.67 (m, 1H), 5.62-5.56 (m, 1H), 5.43-5.40 (m, 1H), 5.38-5.35 (m, 1H), 5.18-5.16 (m, 1H), 4.28-4.21 (m, 1H), 4.18-4.12 (m, 1H), 3.50-3.45 (m, 1H), 3.20-3.12 (m, 1H), 2.49-2.40 (m, 3H), 2.36-2.23 (m, 2H), 1.96-1.82 (m, 2H), 0.93-0.88 (m, 1H), 0.52-0.39 (m, 2H), 0.15-0.08 (m, 1H), 0.06-−0.02 (in, 1H).

### Single-Configuration Compound IV of Compound 1 (0.27 mg)

SFC analysis: retention time: 0.464 minutes, purity: 98%. (Chromatographic column: Chiralcel OD-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 520.1 [M+1].

¹H NMR (400 MHz, CD₃OD) δ 7.71-7.62 (m, 2H), 5.73-5.67 (m, 1H), 5.62-5.56 (m, 1H), 5.41-5.35 (m, 2H), 5.23-5.20 (m, 1H), 4.33-4.23 (m, 1H), 4.21-4.12 (m, 1H), 3.38-3.36 (m, 1H), 3.22-3.13 (m, 1H), 2.44 (s, 3H), 2.37-2.28 (m, 2H), 1.96-1.83 (m, 2H), 0.91-0.86 (m, 1H), 0.50-0.38 (m, 2H), 0.14-0.09 (m, 1H), 0.06-−0.01 (in, 1H).

### Examples 2-2 to 2-6

By referring to the method of Example 2-1, corresponding substrates were used to prepare the following compounds:

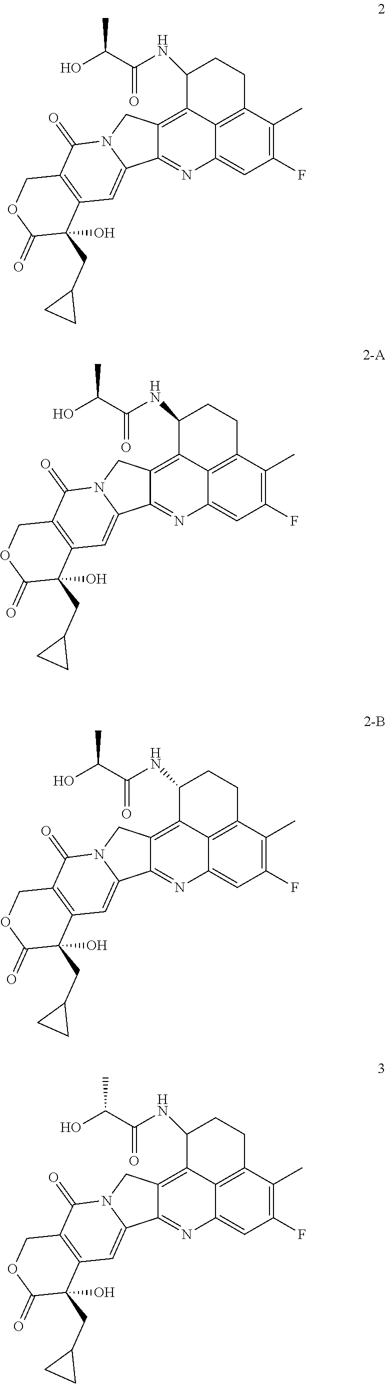

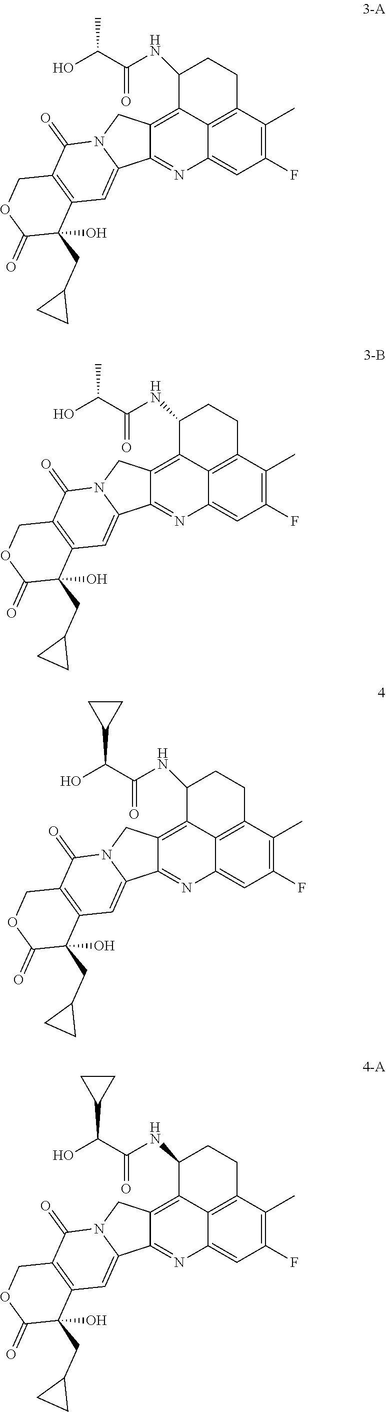

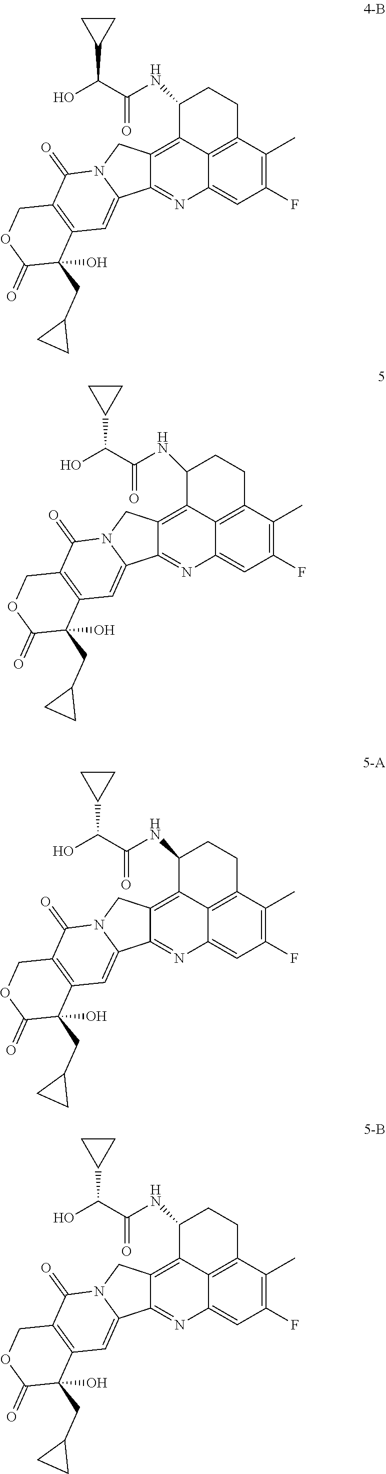

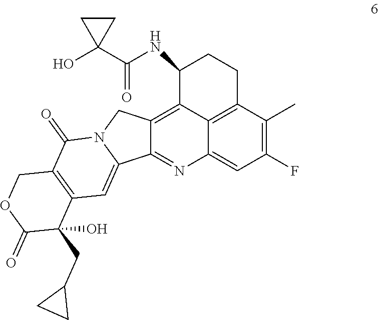 [SMILES: Cc1c(F)cc2nc3c(c4c2c1CC[C@@H]4NC(=O)C1(O)CC1)Cn1c-3cc2c(c1=O)COC(=O)[C@]2(O)CC1CC1]

### Example 2-2: Preparation of Compound 2 and its Isomers 2-A and 2-B

### (S)-N-((1S,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 2-A

### (S)-N-((1R,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 2-B

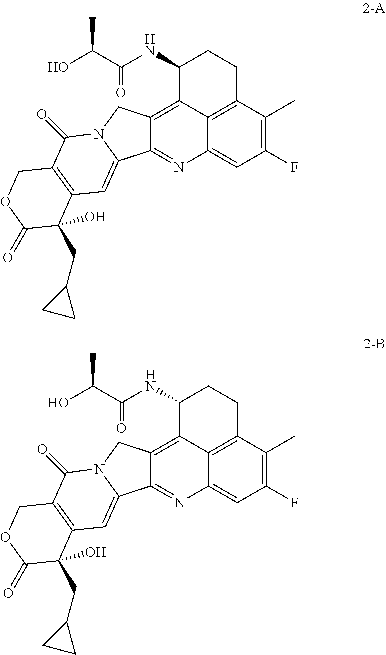

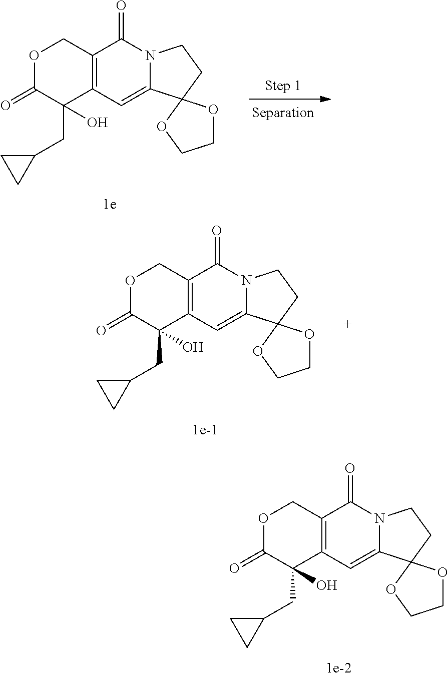 [SMILES: O=C1OCc2c(cc3n(c2=O)CCC32OCCO2)C1(O)CC1CC1 ; O=C1OCc2c(cc3n(c2=O)CCC32OCCO2)[C@@]1(O)CC1CC1 ; O=C1OCc2c(cc3n(c2=O)CCC32OCCO2)[C@]1(O)CC1CC1]

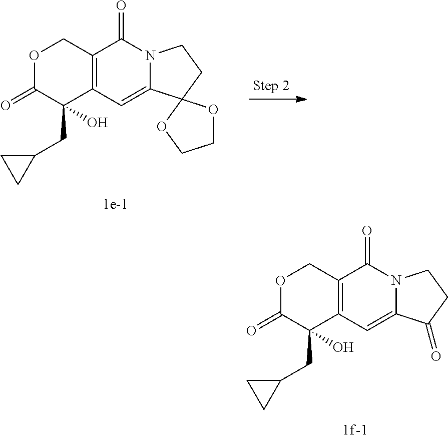 [SMILES: O=C1OCc2c(cc3n(c2=O)CCC32OCCO2)[C@@]1(O)CC1CC1 ; O=C1CCn2c1cc1c(c2=O)COC(=O)[C@]1(O)CC1CC1]

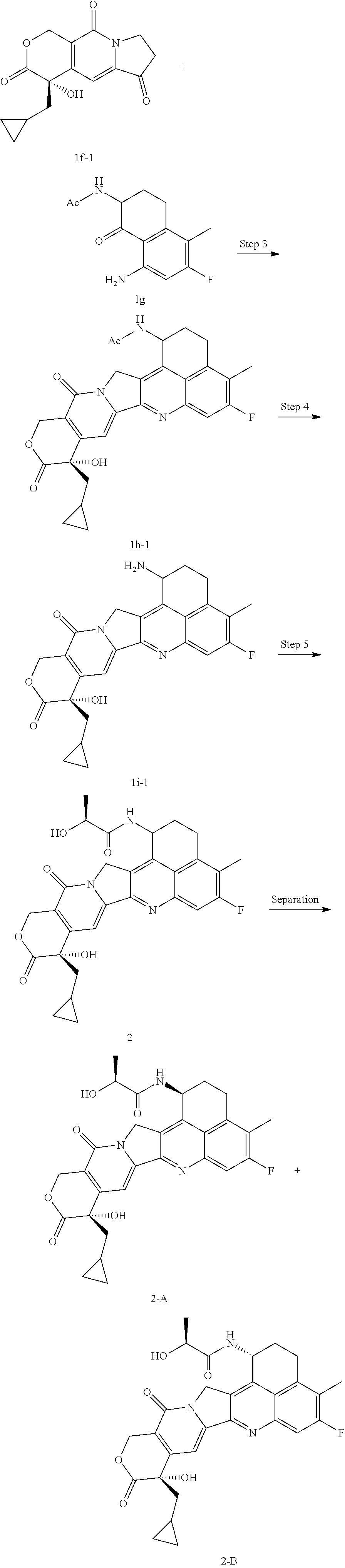

### Step 1

### (S)-4-(Cyclopropylmethyl)-4-hydroxy-1,4,7,8-tetrahydro-3H,10H-spiro[pyrano[3,4-J]indolizine-6,2′-[1,3]dioxolane]-3,10-dione 1e-1

### (R)-4-(Cyclopropylmethyl)-4-hydroxy-1,4,7,8-tetrahydro-3H,10H-spiro[pyrano[3,4-f]indolizine-6,2′-[1,3]dioxolane]-3,10-dione 1e-2

1e (1.30 g, 3.88 mmol) was separated by SFC (separation conditions: chromatographic column: DAICEL CHIRALPAK AS 250 mm×50 mm, 10 μm; mobile phase: A-carbon dioxide: B-methanol (0.1% NH₃—H₂O), isocratic elution: B: 20%, flow rate: 120 mL/min, instrument: Shimadzu LC-30ADsf), to obtain the title product 1e-1 (301 mg, yield: 22.1%) in the form of yellow solid, and the title product 1e-2 (285 mg, yield: 19.4%) in the form of yellow solid.

### Single-Configuration Compound 1e-1

SFC analysis: retention time: 1.392 minutes. (Chromatographic column: Chiralpak AS-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 334.0 [M+1].

### Single-Configuration Compound 1e-2

SFC analysis: retention time: 1.762 minutes. (Chromatographic column: Chiralpak AS-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 333.9 [M+1].

### Step 2

### (S)-4-(Cyclopropylmethyl)-4-hydroxy-7,8-dihydro-1H-pyrano[3,4-f]indolizine-3,6,10(4H)-trione 1f-1

1e-1 (301 mg, 857 μmol) was dissolved in a mixed solution of 2 mL of trifluoroacetic acid and 0.5 mL of water, and stirred at 25° C. for 4 hours. The reaction solution was concentrated by vacuum distillation, and the resultant residue without purification was used to obtain the crude title product

1f-1 (209 mg) in the form of yellow solid. The product was used directly in the next step without purification.

MS m/z (ESI): 290.1 [M+1].

### Step 3

### N-((9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)acetamide 1h-1

1f-1 (50.0 mg, 159 μmol), 1 g (48.0 mg, 192 μmol) and pyridinium 4-toluenesulfonate (8.0 mg, 31.8 μmol) were dissolved in 2 mL of toluene, and stirred at 120° C. for 16 hours under nitrogen protection. The reaction solution was filtered through Celite, the filtrate was added with 20 mL of water and extracted with dichloromethane (15 mL×3). The organic phase was dried over anhydrous sodium sulfate and filtered, and the filtrate was concentrated under reduced pressure. The residue was purified by preparative thin layer chromatography using the eluent system A to obtain the title product 1 h-1 (190 mg, yield: 56%) in the form of yellow solid.

MS m/z (ESI): 504.1 [M+1].

### Step 4

### (9S)-1-Amino-9-(cyclopropylmethyl)-5-fluoro-9-hydroxy-4-methyl-1,2,3,9,12,15-hexahydro-10H,13H-benzo[de]pyrano[3′,4′:6,7]indolizino[1,2-b]quinoline-10,13-dione 1i-1

1h-1 (60.0 mg, 111 μmol) was dissolved in 4.5 mL of 1,2-dimethoxyethane, added with 1.5 mL of methanesulfonic acid and 1.5 mL of water, and stirred at 85° C. for 16 hours. The reaction solution was added dropwise to 10 mL of water under stirring and extracted with dichloromethane (10 mL×5), the organic phase was washed with 25 mL of 0.05 M hydrochloric acid aqueous solution, the aqueous phase was filtered, all the extracted aqueous phases were combined, the combined aqueous phase was adjusted to pH 7-8 with saturated potassium bicarbonate solution at 0° C. and extracted with a mixed solvent (dichloromethane/methanol: 20/3, 15 mL×5). The organic phase was dried over anhydrous sodium sulfate and filtered, and the filtrate was concentrated under reduced pressure to obtain the crude title product 1i-1 (22 mg) in the form of brown solid. The product was used directly in the next step without purification.

MS m/z (ESI): 462.1 [M+1].

### Step 5

### (S)-N-((1S,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 2-A

### (S)-N-((1R,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 2-B

The mesylate salt of 1i-1 (22.0 mg, 47.7 μmol) and (2S)-2-hydroxypropionic acid (6.4 mg, 71.5 μmol) were dissolved in 1.5 mL of N,N-dimethylformamide, then added with 1-hydroxybenzotriazole (9.7 mg, 71.5 μmol), N,N-diisopropylethylamine (18.5 mg, 143 μmol) and 1-(3-dimethylaminopropyl)-3-ethylcarbodiimide hydrochloride (13.7 mg, 71.5 μmol) in sequence, and stirred at 25° C. for 4 hours. The reaction solution was added with toluene (4 mL×2), concentrated under reduced pressure at 25° C. The residue was diluted to 1.5 mL with N,N-dimethylformamide. The diluted solution was purified by pre-HPLC (chromatographic column: Phenomenex luna C18 150×25 mm×10 μm; mobile phase: A-water (0.225% formic acid), B-acetonitrile; gradient elution: B %: 26%-56%) to obtain a mixture (10 mg), which was further purified by supercritical fluid chromatography to obtain the two single-configuration title products 2-A and 2-B:

### Single-Configuration Compound 2-A of Compound 2 (5.55 mg, Yield: 20%, Off-White Solid)

SFC analysis: retention time: 1.381 minutes, purity: 92%. (Chromatographic column: Chiralcel OJ-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 534.1 [M+1].

¹H NMR (400 MHz, CD₃OD) δ 7.61 (s, 1H), 7.53-7.49 (m, 1H), 5.65-5.54 (m, 2H), 5.36-5.29 (m, 1H), 5.25-5.18 (m, 1H), 4.81-4.77 (m, 1H), 4.41-4.33 (m, 1H), 3.28-3.23 (m, 1H), 3.15-3.05 (m, 1H), 2.36-2.33 (m, 3H), 2.33-2.27 (m, 1H), 2.17-2.10 (m, 1H), 1.95-1.87 (m, 1H), 1.85-1.77 (m, 1H), 1.47-1.44 (m, 3H), 0.91-0.87 (m, 1H), 0.51-0.35 (m, 2H), 0.15-0.06 (m, 1H), 0.04-−0.05 (m, 1H).

### Single-Configuration Compound 2-B of Compound 2 (4.06 mg, Yield: 16%, Yellow Solid)

SFC analysis: retention time: 1.299 minutes, purity: 97%. (Chromatographic column: Chiralcel OJ-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 534.1 [M+1].

¹H NMR (400 MHz, CD₃OD) δ 7.61 (s, 1H), 7.57-7.51 (m, 1H), 5.65-5.52 (m, 2H), 5.42-5.30 (m, 2H), 5.12-5.02 (m, 1H), 4.38-4.30 (m, 1H), 3.29-3.24 (m, 1H), 3.20-3.07 (m, 1H), 2.43-2.37 (m, 3H), 2.35-2.26 (m, 2H), 1.97-1.80 (m, 2H), 1.64-1.58 (m, 3H), 0.92-0.83 (m, 1H), 0.51-0.36 (m, 2H), 0.16-0.07 (m, 1H), 0.06-−0.02 (m, 1H).

### Example 2-3: Preparation of Compound 3 and its Isomers 3-A and 3-B

### (R)-N-((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 3-A

### (R)-N-((1R,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 3-B

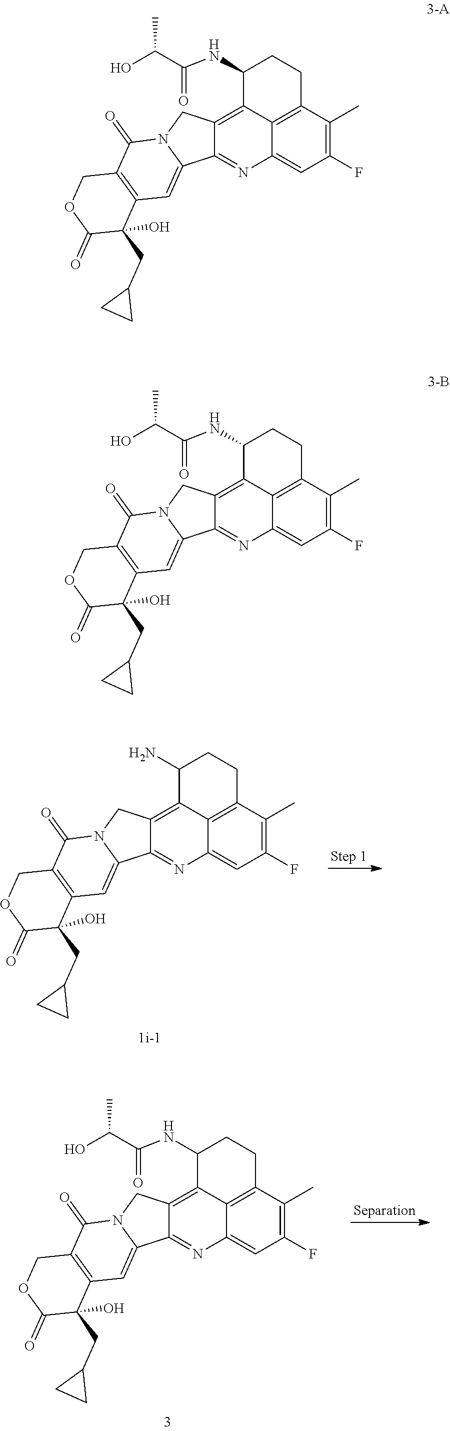 [SMILES: Cc1c(F)cc2nc3c(c4c2c1CC[C@@H]4NC(=O)[C@@H](C)O)Cn1c-3cc2c(c1=O)COC(=O)[C@]2(O)CC1CC1 ; Cc1c(F)cc2nc3c(c4c2c1CC[C@H]4NC(=O)[C@@H](C)O)Cn1c-3cc2c(c1=O)COC(=O)[C@]2(O)CC1CC1 ; Cc1c(F)cc2nc3c(c4c2c1CCC4N)Cn1c-3cc2c(c1=O)COC(=O)[C@]2(O)CC1CC1 ; Cc1c(F)cc2nc3c(c4c2c1CCC4NC(=O)[C@@H](C)O)Cn1c-3cc2c(c1=O)COC(=O)[C@]2(O)CC1CC1]

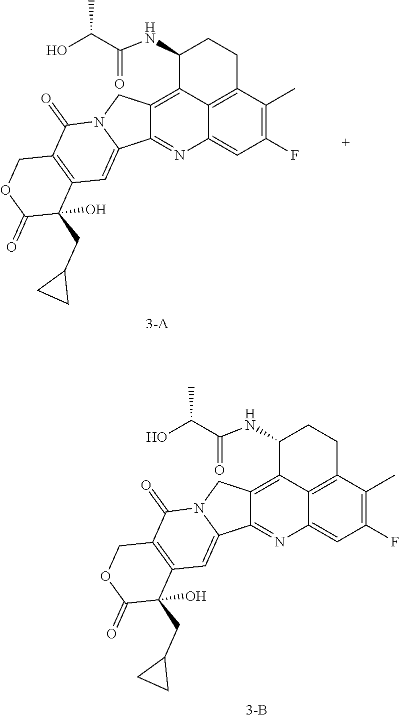 [SMILES: Cc1c(F)cc2nc3c(c4c2c1CC[C@@H]4NC(=O)[C@@H](C)O)Cn1c-3cc2c(c1=O)COC(=O)[C@]2(O)CC1CC1 ; Cc1c(F)cc2nc3c(c4c2c1CC[C@H]4NC(=O)[C@@H](C)O)Cn1c-3cc2c(c1=O)COC(=O)[C@]2(O)CC1CC1]

### Step 1

### (R)-N-((1S,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 3-A

### (R)-N-((1R,9S)-9-(Cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxypropanamide 3-B

1i-1 (40.0 mg, 80.8 μmol) and (2R)-2-hydroxypropionic acid (10.9 mg, 121 μmol) were dissolved in 1.5 mL of N,N-dimethylformamide, then added with 1-hydroxybenzotriazole (16.4 mg, 121 μmol), N,N-diisopropylethylamine (31.3 mg, 242 μmol) and 1-(3-dimethylaminopropyl)-3-ethylcarbodiimide hydrochloride (23.2 mg, 121 μmol) in sequence, and stirred at 25° C. for 4 hours. The reaction solution was added with toluene (4 mL×2), and concentrated under reduced pressure at 25° C. The residue was diluted to 1.5 mL with N,N-dimethylformamide. The diluted solution was purified by pre-HPLC (chromatographic column: Xtimate C18 150×40 mm×10 μm; mobile phase: A-water (0.225% formic acid), B-acetonitrile; gradient elution: B %: 23%-53%), and the obtained mixture was purified by supercritical fluid chromatography to obtain the two single-configuration title products 3-A and 3-B:

### Single-Configuration Compound 3-A of Compound 3 (6.11 mg, Yield: 14%, Off-White Solid)

SFC analysis: retention time: 1.413 minutes, purity: 97%. (Chromatographic column: Chiralcel OJ-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 534.3 [M+1].

¹H NMR (400 MHz, CD₃OD) δ 7.61 (s, 1H), 7.54-7.48 (m, 1H), 5.70-5.62 (m, 1H), 5.59-5.51 (m, 1H), 5.38-5.30 (m, 1H), 5.29-5.21 (m, 1H), 4.83-4.78 (m, 1H), 4.33-4.25 (m, 1H), 3.30-3.23 (m, 1H), 3.20-3.09 (m, 1H), 2.40-2.36 (m, 3H), 2.35-2.31 (m, 1H), 2.25-2.14 (m, 1H), 1.95-1.88 (m, 1H), 1.86-1.79 (m, 1H), 1.60-1.54 (m, 3H), 0.95-0.82 (m, 1H), 0.52-0.35 (m, 2H), 0.15-0.07 (m, 1H), 0.04-−0.04 (m, 1H).

### Single-Configuration Compound 3-B of Compound 3 (2.78 mg, Yield: 6%, Off-White Solid)

SFC analysis: retention time: 2.013 minutes, purity: 97%. (Chromatographic column: Chiralcel OD-3 50×4.6 mm I.D., 3 μm, mobile phase: A-carbon dioxide, B-methanol (0.05% diethylamine), gradient elution: B %: 5%-40%, flow rate: 3 mL/min, instrument: Shimadzu LC-30ADsf).

MS m/z (ESI): 534.1 [M+1].

¹H NMR (400 MHz, DMSO-d6) δ 8.42-8.36 (m, 1H), 7.81-7.75 (m, 1H), 7.37 (s, 1H), 6.57 (s, 1H), 5.58-5.49 (m, 2H), 5.41 (s, 2H), 5.26-5.12 (m, 2H), 4.17-4.09 (m, 1H), 3.19-3.14 (m, 1H), 2.42-2.36 (m, 3H), 2.23-2.10 (m, 2H), 1.90-1.81 (m, 1H), 1.79-1.71 (m, 1H), 1.32-1.27 (m, 3H), 0.85-0.77 (m, 1H), 0.39-0.27 (m, 2H), 0.10-0.02 (m, 1H), −0.03-−0.11 (m, 1H).

### Example 2-4: Preparation of Compound 4 and its Isomers 4-A and 4-B

### (S)-2-Cyclopropyl-N-((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 4-A

### (S)-2-cyclopropyl-N-((1R,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 4-B

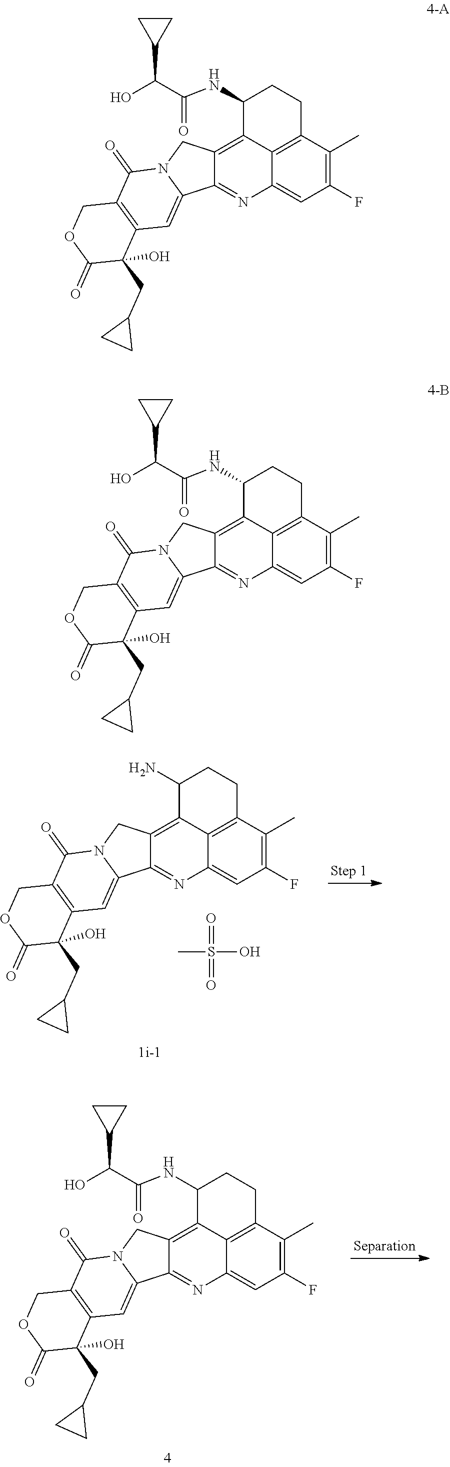

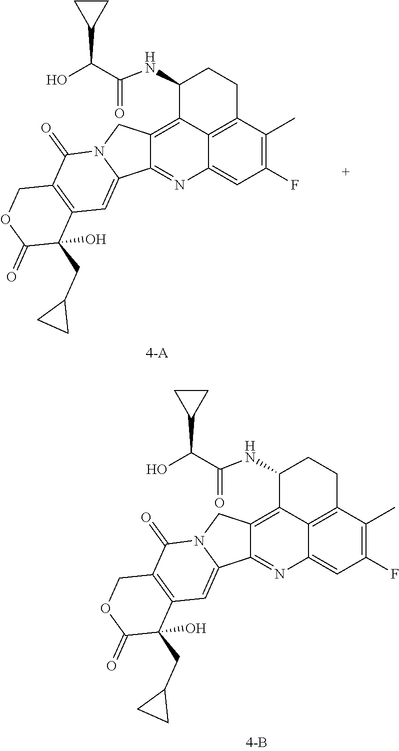 [SMILES: Cc1c(F)cc2nc3c(c4c2c1CC[C@@H]4NC(=O)[C@@H](O)C1CC1)Cn1c-3cc2c(c1=O)COC(=O)[C@]2(O)CC1CC1 ; Cc1c(F)cc2nc3c(c4c2c1CC[C@H]4NC(=O)[C@@H](O)C1CC1)Cn1c-3cc2c(c1=O)COC(=O)[C@]2(O)CC1CC1]

### Step 1

The mesylate of 1-1 (37.0 mg, 78.5 μmol) and (2S)-2-cyclopropyl-2-hydroxyacetic acid (9.59 mg, 78.5 μmol) were dissolved in 1 mL of N,N-dimethylformamide, then added with 1-hydroxybenzotriazole (15.9 mg, 118 μmol), N,N-diisopropylethylamine (30.4 mg, 236 μmol) and 1-(3-dimethylaminopropyl)-3-ethylcarbodiimide hydrochloride (22.6 mg, 118 μmol) in sequence, and stirred at 25° C. for 12 hours. The reaction solution was added with ethyl acetate (10 mL) and washed with water (5 mL×2), the organic phase was dried over anhydrous sodium sulfate and filtered, and the filtrate was concentrated under reduced pressure. The residue was purified by pre-HPLC (chromatographic column: Phenomenex luna C18 150×25 mm×10 μm; mobile phase: A-water (0.225% formic acid), B-acetonitrile; gradient elution: B %: 31%-61%) to obtain a mixture in the form of yellow solid (15 mg, yield 31.2%), which was then separated by preparative SFC (chromatographic column: REGIS (s,s) WHELK-O1250×30 mm, 5 μm; mobile phase: A-carbon dioxide (isopropanol), B-acetonitrile; isocratic elution: B: 65%), to obtain the two single-configuration title products 4-A and 4-B:

### Single-Configuration Compound 4-A of Compound 4 (7.0 mg, Yield: 50.2%, Yellow Solid)

SFC analysis: retention time: 0.761 minutes, purity: 98%. (Chromatographic column: (S,S)WHELK-O1 50×4.6 mm I.D., 3.5 μm, mobile phase: A-carbon dioxide, B-isopropanol and acetonitrile (0.05% diethylamine), isocratic elution: B: 65%, flow rate: 3 mL/min, instrument: Shimadzu LC-30ADsf).

MS m/z (ESI): 560.5 [M+1].

¹H NMR (400 MHz, CD₃OD) δ 7.67 (s, 1H), 7.64-7.60 (m, 1H), 5.68-5.62 (m, 1H), 5.61-5.55 (m, 1H), 5.41-5.37 (m, 1H), 5.36-5.33 (m, 1H), 5.06-5.00 (m, 1H), 3.87-3.84 (m, 1H), 3.21-3.11 (m, 2H), 2.43-2.40 (m, 3H), 2.37-2.31 (m, 1H), 2.25-2.18 (m, 1H), 1.96-1.89 (m, 1H), 1.87-1.80 (m, 1H), 1.32-1.28 (m, 1H), 0.92-0.87 (m, 1H), 0.61-0.55 (m, 2H), 0.53-0.45 (m, 3H), 0.44-0.38 (m, 1H), 0.14-0.08 (m, 1H), 0.04-−0.03 (m, 1H).

### Single-Configuration Compound 4-B of Compound 4 (6.0 mg, Yield: 41.9%, Yellow Solid)

SFC analysis: retention time: 1.486 minutes, purity: 96%. (Chromatographic column: (S,S)WHELK-O1 50×4.6 mm I.D., 3.5 μm, mobile phase: A-carbon dioxide, B-isopropanol and acetonitrile (0.05% diethylamine), isocratic elution: B: 65%, flow rate: 3 mL/min, instrument: Shimadzu LC-30ADsf).

MS m/z (ESI): 560.3 [M+1].

¹H NMR (400 MHz, CD₃OD) δ 7.64 (s, 1H), 7.61-7.57 (m, 1H), 5.67-5.62 (m, 1H), 5.59-5.52 (m, 1H), 5.46-5.39 (m, 1H), 5.38-5.32 (m, 1H), 5.21-5.14 (m, 1H), 3.72-3.68 (m, 1H), 3.22-3.11 (m, 2H), 2.42-2.39 (m, 3H), 2.34-2.28 (m, 2H), 1.96-1.89 (m, 1H), 1.87-1.81 (m, 1H), 1.42-1.35 (m, 1H), 0.91-0.85 (m, 1H), 0.69-0.58 (m, 3H), 0.56-0.51 (m, 1H), 0.48-0.40 (m, 2H), 0.15-0.07 (m, 1H), 0.06-−0.02 (m, 1H).

### Example 2-5: Preparation of Compound 5 and its Isomers 5-A and 5-B

### (R)-2-Cyclopropyl-N-((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 5-A

### (R)-2-Cyclopropyl-N-((1R,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide 5-B

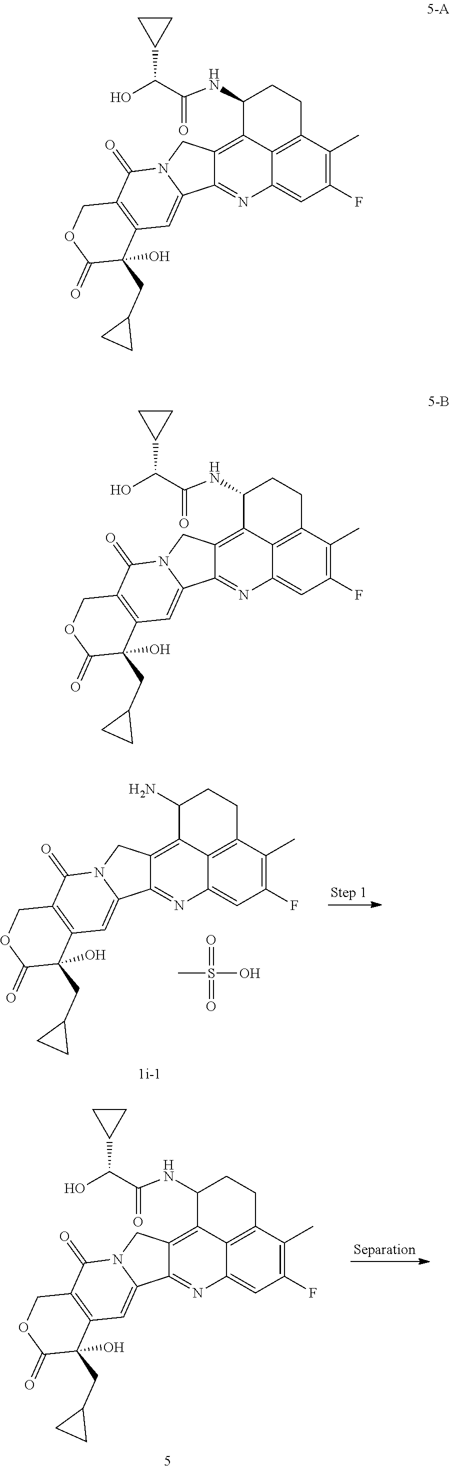

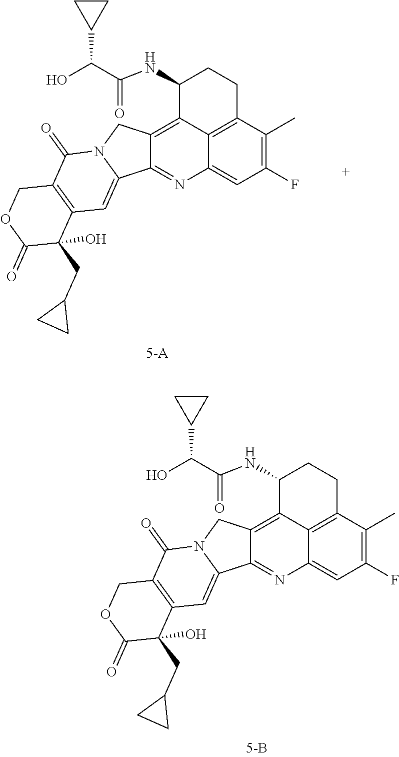 [SMILES: Cc1c(F)cc2nc3c(c4c2c1CC[C@@H]4NC(=O)[C@H](O)C1CC1)Cn1c-3cc2c(c1=O)COC(=O)[C@]2(O)CC1CC1 ; Cc1c(F)cc2nc3c(c4c2c1CC[C@H]4NC(=O)[C@H](O)C1CC1)Cn1c-3cc2c(c1=O)COC(=O)[C@]2(O)CC1CC1]

### Step 1

The mesylate of 1i-1 (25.0 mg, 42.3 μmol) and (2R)-2-cyclopropyl-2-hydroxyacetic acid (5.89 mg, 50.7 μmol) were dissolved in 1.5 mL of N,N-dimethylformamide, then added with 1-hydroxybenzotriazole (8.57 mg, 63.4 μmol), N,N-diisopropylethylamine (16.4 mg, 127 μmol) and 1-(3-dimethylaminopropyl)-3-ethylcarbodiimide hydrochloride (12.2 mg, 63.4 μmol) in sequence, and stirred at 20° C. for 4 hours. The reaction solution was added with toluene (4 mL×2), and concentrated under reduced pressure at 25° C. The residue was diluted to 1.5 mL with N,N-dimethylformamide. The diluted solution was purified by pre-HPLC (chromatographic column: Phenomenex luna C18 150×25 mm×10 μm; mobile phase: A-water (0.225% formic acid), B-acetonitrile; gradient elution: B %: 28%-58%) to obtain two single-configuration title products 5-A and 5-B:

### Single-Configuration Compound 5-A of Compound 5 (4.95 mg, Yield: 20%, Off-White Solid)

SFC analysis: retention time: 2.115 minutes, purity: 97%. (Chromatographic column: Chiralpak IC-3 50×4.6 mm I.D., 3 μm, mobile phase: A-carbon dioxide, B-methanol and acetonitrile (0.05% diethylamine), isocratic elution: B: 50%, flow rate: 3 mL/min, instrument: Shimadzu LC-30ADsf).

MS m/z (ESI): 560.1 [M+1].

¹H NMR (400 MHz, CD₃OD) δ 7.70-7.64 (m, 2H), 5.70-5.65 (m, 1H), 5.60-5.54 (m, 1H), 5.46-5.38 (m, 1H), 5.37-5.32 (m, 1H), 5.23-5.17 (m, 1H), 3.72-3.67 (m, 1H), 3.36-3.34 (m, 1H), 3.23-3.17 (m, 1H), 2.48-2.42 (m, 3H), 2.37-2.31 (m, 1H), 2.31-2.25 (m, 1H), 1.96-1.89 (m, 1H), 1.88-1.80 (m, 1H), 1.34-1.31 (m, 1H), 0.92-0.89 (m, 1H), 0.68-0.42 (m, 6H), 0.15-0.07 (m, 1H), 0.05-−0.02 (m, 1H).

### Single-Configuration Compound 5-B of Compound 5 (1.95 mg, Yield: 8%, White Solid)

SFC analysis: retention time: 1.179 minutes, purity: 97%. (Chromatographic column: Chiralpak IC-3 50×4.6 mm I.D., 3 μm, mobile phase: A-carbon dioxide, B-methanol and acetonitrile (0.05% diethylamine), isocratic elution: B: 50%, flow rate: 3 mL/min, instrument: Shimadzu LC-30ADsf).

MS m/z (ESI): 560.1 [M+1].

¹H NMR (400 MHz, CD₃OD) δ 7.69 (s, 1H), 7.69-7.62 (m, 1H), 5.69-5.63 (m, 1H), 5.61-5.55 (m, 1H), 5.49-5.42 (m, 1H), 5.40-5.34 (m, 1H), 5.24-5.16 (m, 1H), 3.91-3.84 (m, 1H), 3.38-3.36 (m, 1H), 3.24-3.16 (m, 1H), 2.44 (s, 3H), 2.40-2.23 (m, 2H), 1.97-1.80 (m, 2H), 1.29-1.21 (m, 1H), 0.95-0.85 (m, 1H), 0.64-0.36 (m, 6H), 0.17-0.07 (m, 1H), 0.07-−0.02 (m, 1H).

### Example 2-6: Preparation of Compound 6

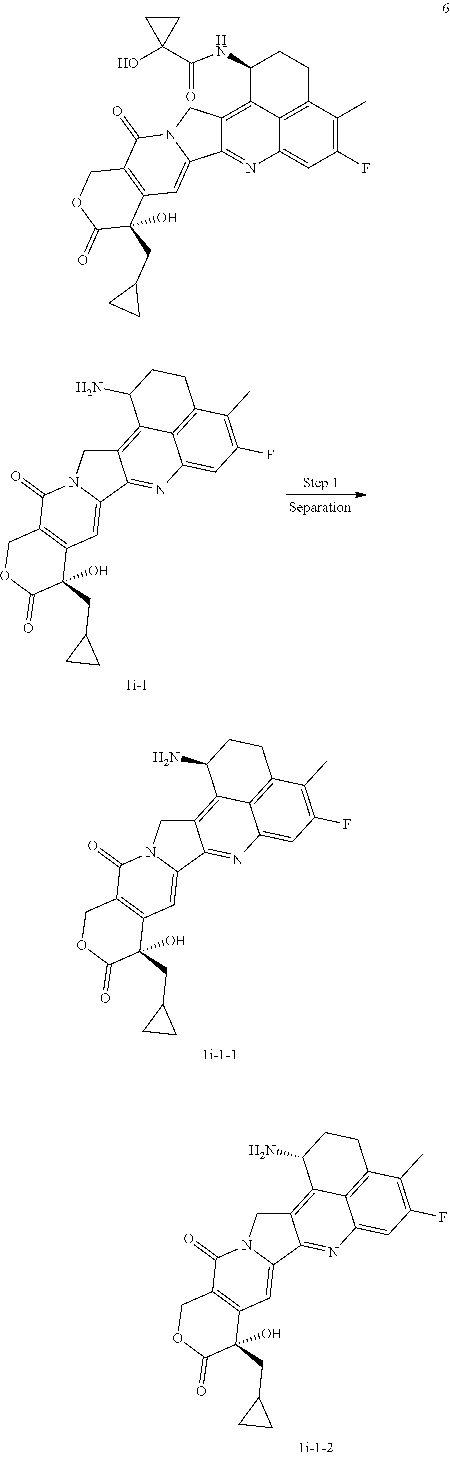 [SMILES: Cc1c(F)cc2nc3c(c4c2c1CC[C@@H]4NC(=O)C1(O)CC1)Cn1c-3cc2c(c1=O)COC(=O)[C@]2(O)CC1CC1 ; Cc1c(F)cc2nc3c(c4c2c1CCC4N)Cn1c-3cc2c(c1=O)COC(=O)[C@]2(O)CC1CC1 ; Cc1c(F)cc2nc3c(c4c2c1CC[C@@H]4N)Cn1c-3cc2c(c1=O)COC(=O)[C@]2(O)CC1CC1 ; Cc1c(F)cc2nc3c(c4c2c1CC[C@H]4N)Cn1c-3cc2c(c1=O)COC(=O)[C@]2(O)CC1CC1]

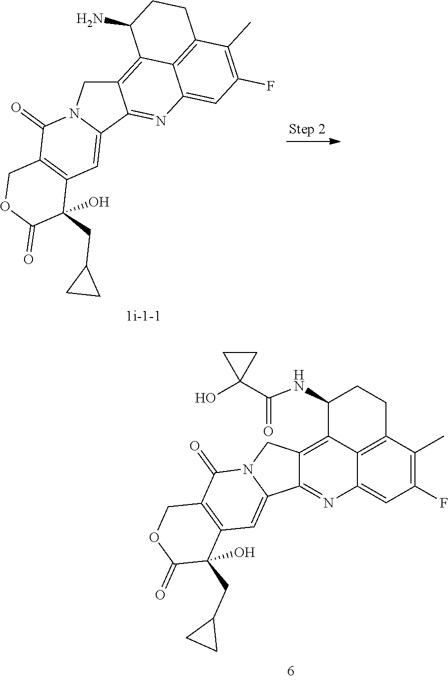 [SMILES: Cc1c(F)cc2nc3c(c4c2c1CC[C@@H]4N)Cn1c-3cc2c(c1=O)COC(=O)[C@]2(O)CC1CC1 ; Cc1c(F)cc2nc3c(c4c2c1CC[C@@H]4NC(=O)C1(O)CC1)Cn1c-3cc2c(c1=O)COC(=O)[C@]2(O)CC1CC1]

### Step 1

### (1S,9S)-1-Amino-9-(cyclopropylmethyl)-5-fluoro-9-hydroxy-4-methyl-1,2,3,9,12,15-hexahydro-10H,13H-benzo[de]pyrano[3′,4′:6,7]indolizino[1,2-b]quinoline-10,13-dione 1i-1-1

### (1R,9S)-1-amino-9-(cyclopropylmethyl)-5-fluoro-9-hydroxy-4-methyl-1,2,3,9,12,15-hexahydro-10H,13H-benzo[de]pyrano[3′,4′:6,7]indolizino[1,2-b]quinoline-10,13-dione 1i-1-2

1i-1 (550 mg, 1.10 mmol) was separated by SFC (separation conditions: chromatographic column: DAICEL CHIRALPAK IC 250 mm×50 mm, 10 μm; mobile phase: A-n-hexane: B-ethanol, isocratic elution: B: 45%), to obtain the title product 1i-1-1 (260 mg, yield: 41%) in the form of yellow solid, and the title product 1i-1-2 (290 mg, yield: 46%) in the form of yellow solid.

Single-Configuration Compound 1i-1-1

SFC analysis: retention time: 3.039 minutes. (Chromatographic column: Chiralpak IC-3 50×4.6 mm I.D., 3 μm, mobile phase: A-n-hexane (0.05% isopropylamine), B-ethanol and acetonitrile (0.05% isopropylamine), isocratic elution: B: 45%, flow rate: 1 mL/min, instrument: Shimadzu LC-20AD).

MS m/z (ESI): 462.3 [M+1].

Single-Configuration Compound 1i-1-2

SFC analysis: retention time: 4.951 minutes. (Chromatographic column: Chiralpak IC-3 50×4.6 mm I.D., 3 μm, mobile phase: A-n-hexane (0.05% isopropylamine), B-ethanol and acetonitrile (0.05% isopropylamine), isocratic elution: B: 45%, flow rate: 1 mL/min, instrument: Shimadzu LC-20AD).

MS m/z (ESI): 462.3 [M+1].

### Step 2

1i-1-1 (13 mg, 25.35 μmol), 1-hydroxycyclopropanecarboxylic acid (4 mg, 38.03 μmol) and 0-(7-azabenzotriazol-1-yl)-N,N,N′,N′-tetramethyluronium hexafluorophosphate (12 mg, 30.42 μmol) were dissolved in 1 mL of N,N-dimethylformamide, added with N,N-diisopropylethylamine (10 mg, 76.06 μmol), and stirred at 25° C. for 1 hour. The reaction solution was purified by pre-HPLC (chromatographic column: InfinityLab Poroshell 120 SB-C18 21.2×150 mm, 4 μm; mobile phase: A-water (0.1% formic acid), B-acetonitrile; gradient elution: B %: 20%-80%) to obtain the title product 6 (2.25 mg, yield: 16%) in the form of white solid.

MS m/z (ESI): 546.3 [M+1].

¹H NMR (400 MHz, DMSO-d6) δ 8.66-8.58 (m, 1H), 7.80-7.74 (m, 1H), 7.41-7.34 (m, 1H), 6.62-6.58 (m, 1H), 6.37-6.29 (m, 1H), 5.61-5.53 (m, 1H), 5.48-5.37 (m, 2H), 5.33-5.24 (m, 1H), 5.12-5.04 (m, 1H), 3.28-3.22 (m, 1H), 3.17-3.06 (m, 1H), 2.44-2.33 (m, 3H), 2.28-2.15 (m, 2H), 1.86-1.73 (m, 2H), 1.27-1.15 (m, 2H), 1.00-0.89 (m, 2H), 0.87-0.75 (m, 1H), 0.40-0.25 (m, 2H), 0.08-0.03 (m, 1H), −0.06-−0.14 (m, 1H).

### Examples 2-7 to 2-13

By referring to the method of Example 2-1, corresponding substrates were used to prepare the following compounds:

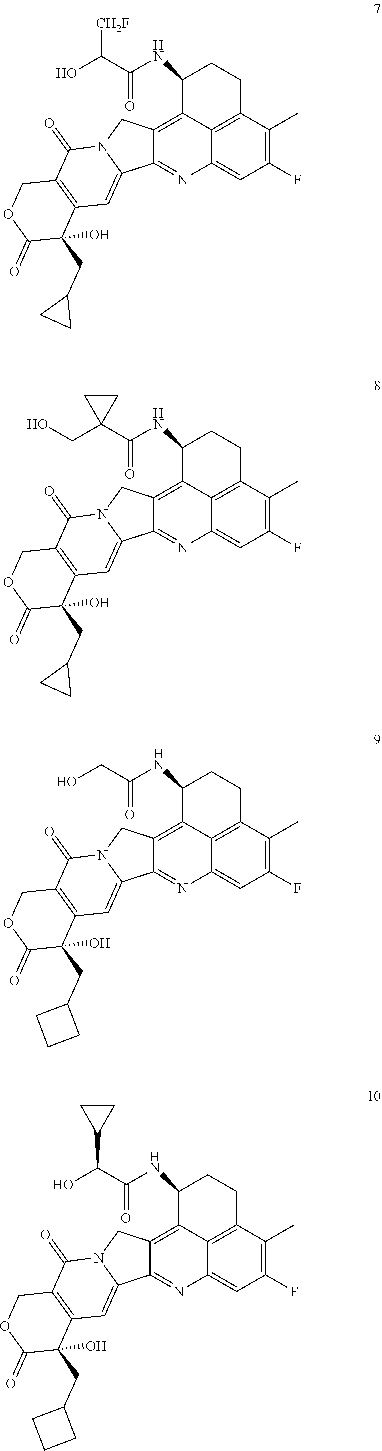

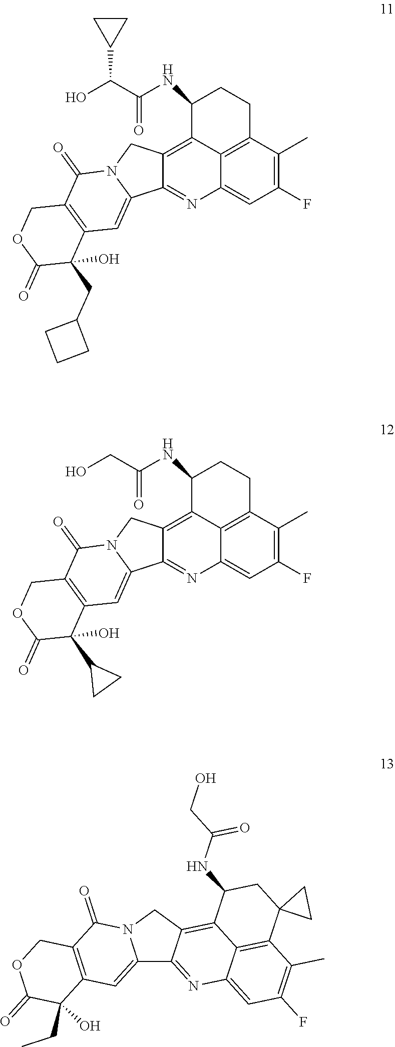 [SMILES: Cc1c(F)cc2nc3c(c4c2c1CC[C@@H]4NC(=O)[C@H](O)C1CC1)Cn1c-3cc2c(c1=O)COC(=O)[C@]2(O)CC1CCC1 ; Cc1c(F)cc2nc3c(c4c2c1CC[C@@H]4NC(=O)CO)Cn1c-3cc2c(c1=O)COC(=O)[C@]2(O)C1CC1 ; CC[C@@]1(O)C(=O)OCc2c1cc1n(c2=O)Cc2c-1nc1cc(F)c(C)c3c1c2[C@@H](NC(=O)CO)CC31CC1]

### Example 2-14: Preparation of Compound 14

### N′-Acetyl-N-((1 S,9SJ-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetohydrazide 14

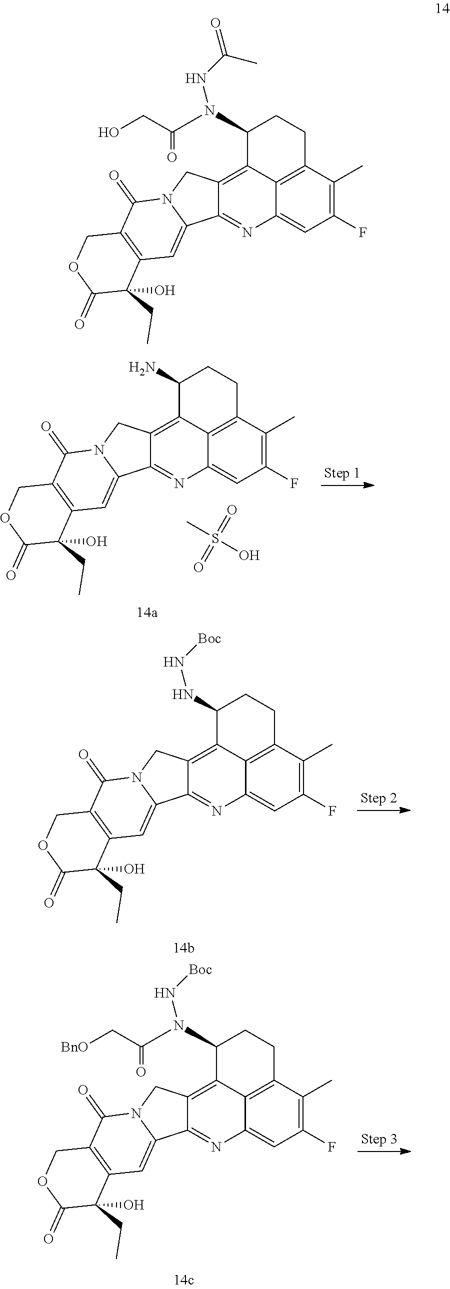

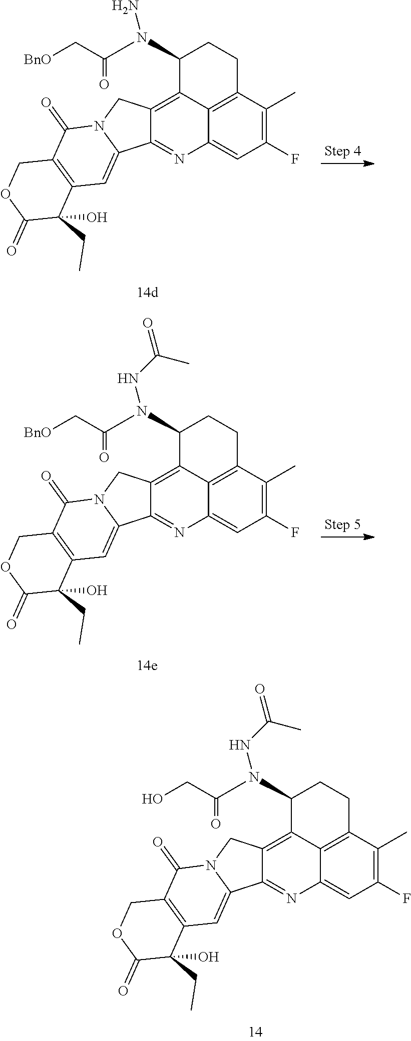 [SMILES: CC[C@@]1(O)C(=O)OCc2c1cc1n(c2=O)Cc2c-1nc1cc(F)c(C)c3c1c2[C@@H](N(N)C(=O)COCc1ccccc1)CC3 ; CC[C@@]1(O)C(=O)OCc2c1cc1n(c2=O)Cc2c-1nc1cc(F)c(C)c3c1c2[C@@H](N(NC(C)=O)C(=O)COCc1ccccc1)CC3 ; CC[C@@]1(O)C(=O)OCc2c1cc1n(c2=O)Cc2c-1nc1cc(F)c(C)c3c1c2[C@@H](N(NC(C)=O)C(=O)CO)CC3]

### Step 1

### tert-Butyl 2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)hydrazine-1-carboxylate 14b

14a (100 mg, 0.19 mmol) and 2-(tert-butyl) 3,3-diethyl 1,2-oxaziridine-2,3,3-tricarboxylate (54.9 mg, 0.19 mmol, obtained by applying the method disclosed in the literature “Organic Letters, 2005, vol. 7, #4, p. 713-716”) were dissolved in 10 mL of dichloromethane, added with triethylamine (38.4 mg, 0.38 mmol), and stirred at 25° C. for 12 hours. 20 mL of water was added, and the diluted reaction solution was separated. The organic phase was washed with saturated sodium chloride solution (10 mL), the organic phase was dried over anhydrous sodium sulfate and filtered, and the filtrate was concentrated by vacuum distillation. The residue was purified by preparative thin layer chromatography using the eluent system A to obtain the title product 14b (55 mg, yield: 53%) in the form of white solid.

MS m/z (ESI): 551.2 [M+1].

### Step 2

### tert-Butyl 2-(2-(benzyloxy)acetyl)-2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)hydrazine-1-carboxylate 14c

14b (55 mg, 0.1 mmol) was dissolved in 10 mL of tetrahydrofuran, and added with sodium bicarbonate (840 mg, 10 mmol). To the above solution, a tetrahydrofuran solution of 2-benzyloxyacetyl chloride (184 mg, 1.0 mmol, 5 mL) was added dropwise under nitrogen protection at 25° C., and stirred at 25° C. for 1 hour. The reaction solution was poured into 10 mL of water, and extracted with ethyl acetate (10 mL×2). The organic phase was washed with saturated sodium chloride solution (10 mL×2), dried over anhydrous sodium sulfate, and filtered. The filtrate was concentrated by vacuum distillation. The residue was purified by preparative thin layer chromatography using the development system A to obtain the title product 14c (58 mg, yield: 83%) in the form of white solid.

MS m/z (ESI): 699.3 [M+1].

### Step 3

### 2-(Benzyloxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)acetohydrazine 14d

14c (58 mg, 0.09 mmol) was dissolved in 10 mL of dichloromethane, added with 1 mL of trifluoroacetic acid, and stirred at 25° C. for 5 hours. The reaction solution was concentrated by vacuum distillation to obtain the crude title product 14d (70 mg) in the form of light brown solid. The product was used directly in the next step without purification.

MS m/z (ESI): 599.2 [M+1].

### Step 4

### N′-Acetyl-2-(benzyloxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) acetohydrazine 14e

14d (70 mg, 0.09 mmol) was dissolved in 10 mL of tetrahydrofuran, and added with sodium bicarbonate (756 mg, 9 mmol). To the above solution, a tetrahydrofuran solution of acetyl chloride (71 mg, 0.9 mmol, 3 mL) was added dropwise under nitrogen protection at 25° C., and stirred at 25° C. for 1 hour. The reaction solution was poured into 10 mL of water, and extracted with ethyl acetate (10 mL×2). The organic phase was washed with saturated sodium chloride solution (10 mL×2), dried over anhydrous sodium sulfate, and filtered. The filtrate was concentrated by vacuum distillation. The residue was purified by preparative thin layer chromatography using the development system A to obtain the title product 14e (45 mg, yield: 78%) as an off-white solid.

MS m/z (ESI): 641.7 [M+1].

### Step 5

### N′-Acetyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetohydrazide 14

14e (45 mg, 0.07 mmol) was dissolved in 10 mL of tetrahydrofuran, cooled to −10° C. under nitrogen protection in ice-salt bath, added with palladium dichloride (50 mg, 0.282 mmol), and subjected to replacement with hydrogen gas three times. The reaction solution was stirred at 0° C. for 2 hours. The reaction solution was filtered through Celite, and the filtrate was concentrated by vacuum distillation. The residue was purified by preparative HPLC (separation conditions: chromatographic column: Sunfire Prep C18 OBD 10 μm, 19×250 mm, mobile phase: A-acetonitrile/methanol=1/1, B-water (0.2% formic acid), gradient elution, A %: 45%-95%, flow rate: 20 mL/min, instrument: GILSON GX-281) to obtain the title product 14 (4.5 mg, yield: 12%) in the form of off-white solid.

MS m/z (ESI): 551.1 [M+1].

¹H NMR (400 MHz, DMSO-d6) δ 10.22-9.65 (m, 1H), 7.79 (d, J=10.8 Hz, 1H), 7.36-7.28 (m, 1H), 6.58-6.52 (m, 1H), 6.22-5.99 (m, 1H), 5.43 (s, 2H), 5.33 (s, 1H), 5.15-5.00 (m, 2H), 4.26-4.13 (m, 1H), 3.98-3.71 (m, 1H), 3.21-3.04 (m, 2H), 2.42-2.34 (m, 3H), 2.25-2.08 (m, 1H), 1.92-1.82 (m, 2H), 1.67-1.24 (m, 3H), 1.25-1.20 (m, 1H), 0.91-0.84 (in, 3H).

### Examples 2-15 to 2-18, 2-20, 2-21 and 2-23

By referring to the method of Example 2-14, corresponding substrates were used to prepare the following compounds:

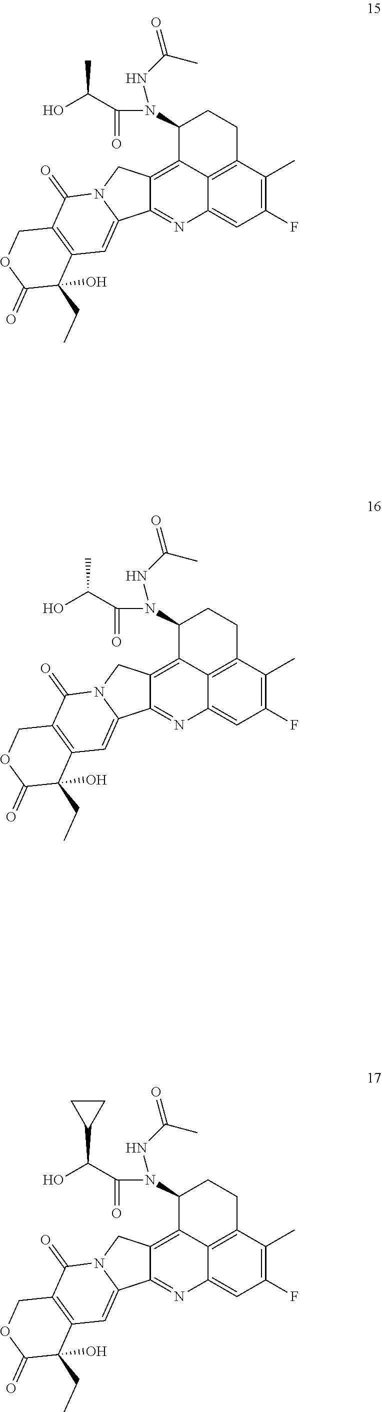 [SMILES: CC[C@@]1(O)C(=O)OCc2c1cc1n(c2=O)Cc2c-1nc1cc(F)c(C)c3c1c2[C@@H](N(NC(C)=O)C(=O)[C@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](O)C1CC1)CC3]

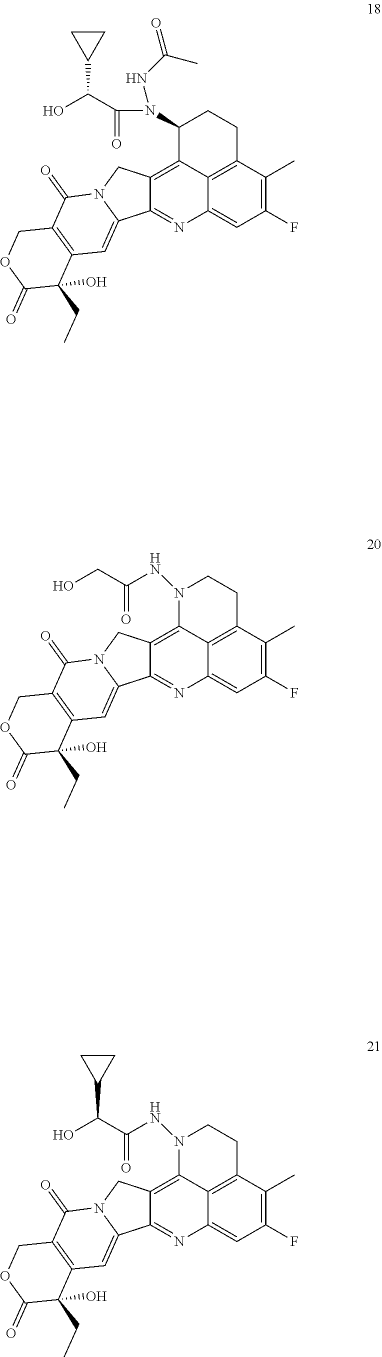 [SMILES: CC[C@@]1(O)C(=O)OCc2c1cc1n(c2=O)Cc2c-1nc1cc(F)c(C)c3c1c2[C@@H](N(NC(C)=O)C(=O)[C@H](O)C1CC1)CC3 ; CC[C@@]1(O)C(=O)OCc2c1cc1n(c2=O)Cc2c-1nc1cc(F)c(C)c3c1c2N(NC(=O)CO)CC3 ; CC[C@@]1(O)C(=O)OCc2c1cc1n(c2=O)Cc2c-1nc1cc(F)c(C)c3c1c2N(NC(=O)[C@@H](O)C1CC1)CC3]

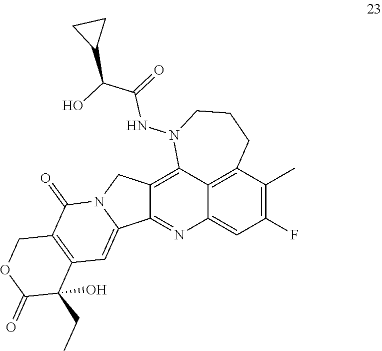 [SMILES: CC[C@@]1(O)C(=O)OCc2c1cc1n(c2=O)Cc2c-1nc1cc(F)c(C)c3c1c2N(NC(=O)[C@@H](O)C1CC1)CCC3]

### Example 2-19: Preparation of Compound 19

### N′-((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-N′-(2-hydroxyacetyl)cyclopropanecarbohydrazide 19

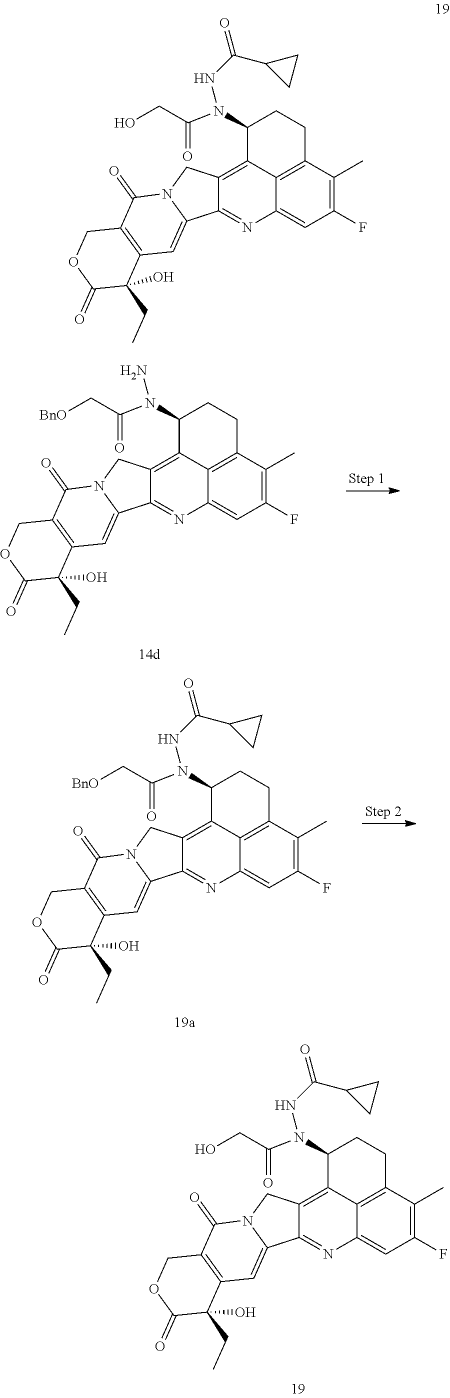

### Step 1

### N′-(2-(Benzyloxy)acetyl)-N′-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) cyclopropanecarbohydrazide 19a

14d (50 mg, 0.08 mmol) was dissolved in 10 mL of tetrahydrofuran, added with sodium bicarbonate (672 mg, 8 mmol), and added dropwise with a tetrahydrofuran solution (5 mL) of cyclopropanecarbonyl chloride (83 mg, 0.8 mmol) under nitrogen protection. The reaction solution was stirred at 25° C. for 16 hours. The reaction solution was poured into water (10 mL) and extracted with ethyl acetate (10 mL×2). The organic phase was washed with saturated sodium chloride solution (10 mL×2), dried over anhydrous sodium sulfate, and filtered. The filtrate was concentrated by vacuum distillation. The residue was purified by pre-HPLC (separation conditions: chromatographic column: Sunfire Prep C18 OBD 10 μm, 19×250 mm, mobile phase: A-acetonitrile/methanol=1/1, B-water (0.2% formic acid), gradient elution, A %: 45%-95%, flow rate: 20 mL/min, instrument: GILSON GX-281), and lyophilized to obtain the title product 19a (19 mg, yield: 34%) in the form of off-white solid.

MS m/z (ESI): 667.2 [M+1].

### Step 2

### N′-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-N′-(2-hydroxyacetyl)cyclopropanecarbohydrazide 19

19a (19 mg, 0.03 mmol) was dissolved in 5 mL of tetrahydrofuran under nitrogen protection, cooled to −10° C. in ice-salt bath, added with palladium dichloride (20 mg, 0.113 mmol), and subjected to replacement with hydrogen gas three times. The reaction solution was stirred at 0° C. for 15 hours. The reaction solution was filtered through Celite, and the filtrate was concentrated by vacuum distillation. The residue was purified by pre-HPLC (separation conditions: chromatographic column: Sunfire Prep C18 OBD 10 μm, 19×250 mm, mobile phase: A-acetonitrile/methanol=1/1, B-water (0.2% formic acid), gradient elution, A %: 45%-95%, flow rate: 20 mL/min, instrument: GILSON GX-281) to obtain the title product 19 (4.2 mg, yield: 26%) in the form of white solid.

MS m/z (ESI): 577.2 [M+1].

### Example 2-22: Preparation of Compound 22

### (S)-N-(10-Ethyl-6-fluoro-10-hydroxy-5-methyl-11,14-dioxo-3,4,10,11,14,16-hexahydro-13H-azepino[2,3,4-de]pyrano[3′,4′:6,7]indolizino[1,2-b]quinolin-1(2H)-yl)-2-hydroxyacetamide 22

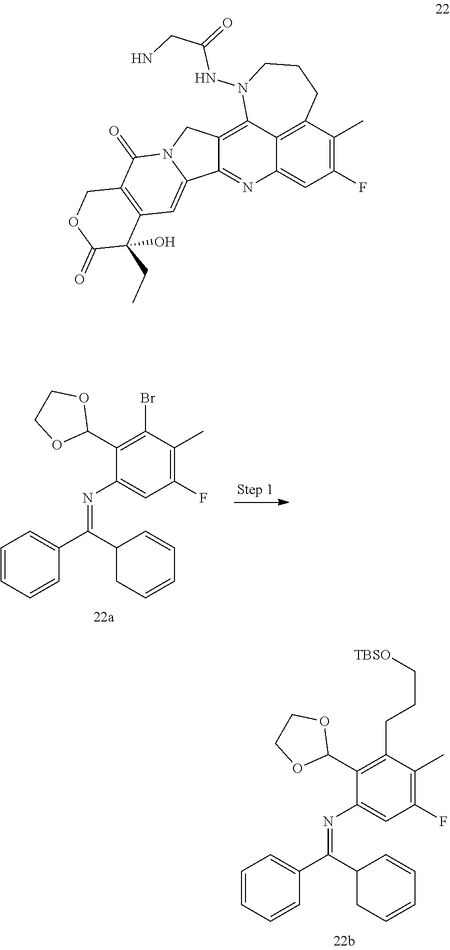 [SMILES: CC[C@@]1(O)C(=O)OCc2c1cc1n(c2=O)Cc2c-1nc1cc(F)c(C)c3c1c2N(NC(=O)CN)CCC3 ; Cc1c(F)cc(/N=C(/c2ccccc2)C2C=CC=CC2)c(C2OCCO2)c1Br ; Cc1c(F)cc(/N=C(/c2ccccc2)C2C=CC=CC2)c(C2OCCO2)c1CCCO[Si](C)(C)C(C)(C)C]

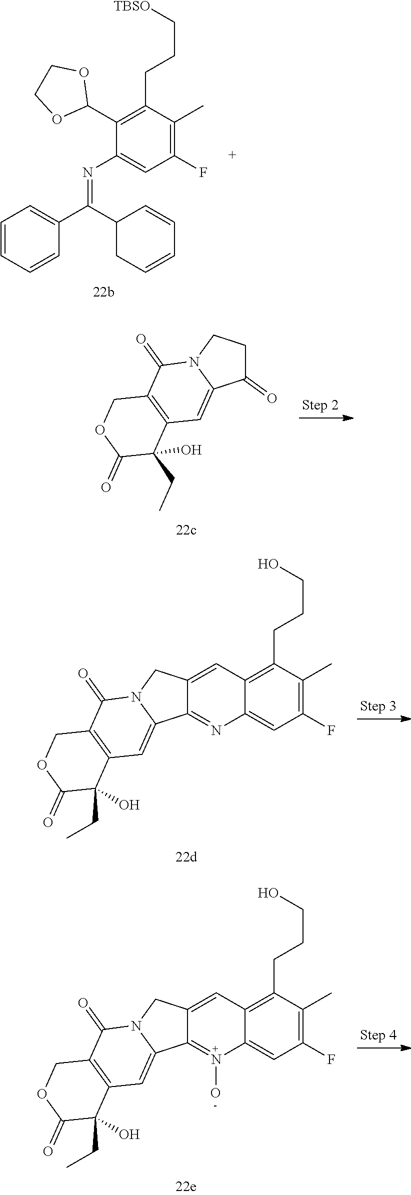

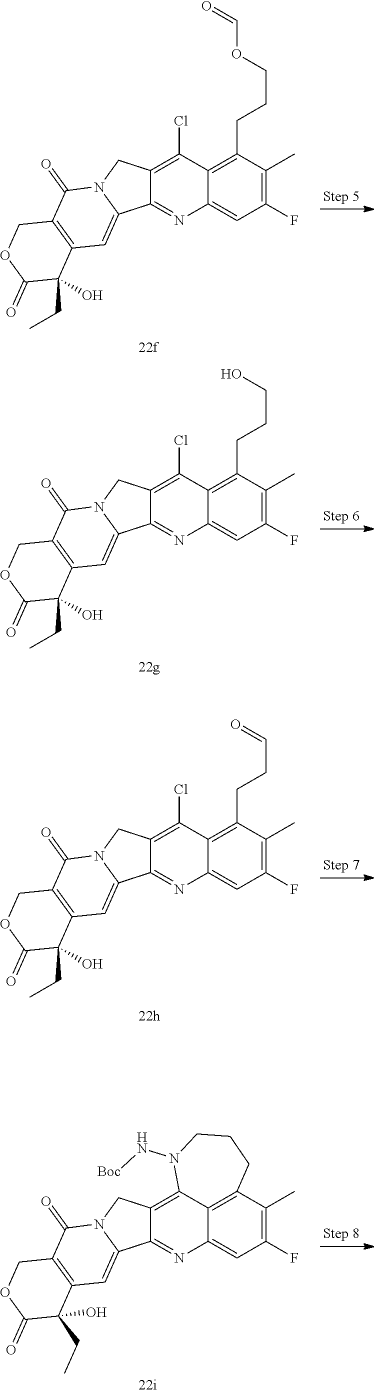 [SMILES: CC[C@@]1(O)C(=O)OCc2c1cc1n(c2=O)Cc2c-1nc1cc(F)c(C)c(CCCOC=O)c1c2Cl ; CC[C@@]1(O)C(=O)OCc2c1cc1n(c2=O)Cc2c-1nc1cc(F)c(C)c(CCCO)c1c2Cl ; CC[C@@]1(O)C(=O)OCc2c1cc1n(c2=O)Cc2c-1nc1cc(F)c(C)c(CCC=O)c1c2Cl ; CC[C@@]1(O)C(=O)OCc2c1cc1n(c2=O)Cc2c-1nc1cc(F)c(C)c3c1c2N(NC(=O)OC(C)(C)C)CCC3]

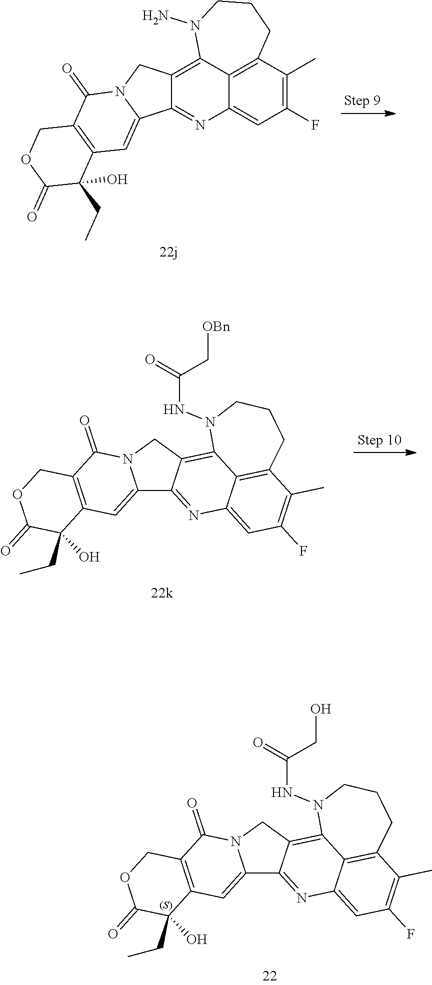

### Step 1

### N-(3-(3-((tert-butyldimethylsilyl)oxy)propyl)-2-(1,3-dioxolan-2-yl)-5-fluoro-4-methylpheny 1)-1,1-diphenylmethanimine 22b

Allyloxy-tert-butyl-dimethylsilane (6.89 g, 36.00 mmol, 90% purity, obtained by applying the method disclosed in the literature “Organic Letters, 2017, vol. 19, #11, p. 2869-2872) was dissolved in 70 mL of toluene under protection of nitrogen gas, cooled to 10° C. in ice-water bath, and added with 9-borabicyclo[3.3.1]nonane (0.5 M, 86.41 mL, 43.20 mmol). The reaction solution was stirred at 80° C. for 20 minutes, and the completion of the reaction was monitored by TLC. The reaction solution was cooled to 10° C. in ice-water bath, and sodium hydroxide (2.88 g, 72.01 mmol, 24 mL) aqueous solution, tetrabutylammonium iodide (664.95 mg, 1.80 mmol), 22a (5.02 g, 10.80 mmol) and 1,1′-bis(diphenylphosphino)ferrocene(II) palladium dichloride (526.89 mg, 720.09 μmol) were added to the reaction solution in sequence under nitrogen protection, and stirred at 80° C. for 15 hours. The completion of the reaction was monitored by LC-MS. 100 mL of water was added to dilute the reaction solution, and the reaction solution was extracted with dichloromethane (100 mL×3). The organic phase was washed with water (100 mL×2), dried over anhydrous sodium sulfate and filtered. The filtrate was concentrated by vacuum distillation. The obtained residue was purified by reverse phase high performance liquid chromatography to obtain the title product 22b (2.60 g, yield: 12%, purity: 88.75%) in the form of yellow colloid.

MS m/z (ESI): 534.5 [M+1].

### Step 2

### (S)-4-Ethyl-8-fluoro-4-hydroxy-10-(3-hydroxypropyl)-9-methyl-1,12-dihydro-14H-pyrano[3′,4′:6,7]indolizino[1,2-b]quinoline-3,14(4H)-dione 22d

22b (380 mg, 654.99 μmol) was dissolved in 5 mL of ethanol, added with 22c (103.45 mg, 393.00 μmol, prepared by applying the method disclosed in patent application “WO2019238046 A1”) and concentrated hydrochloric acid (12 M, 0.5 mL), and stirred at 80° C. for 2 hours, and the completion of the reaction was monitored by LC-MS. The reaction solution was concentrated by vacuum distillation to remove ethanol, 5 mL of water was added to dilute the reaction solution, the diluted reaction solution was extracted with dichloromethane (10 mL×3), the organic phase was dried over anhydrous sodium sulfate and filtered, and the filtrate was concentrated by vacuum distillation. The residue was purified by silica gel column chromatography using the eluent system A to obtain the title product 22d (80 mg, yield: 27%) in the form of yellow solid.

MS m/z (ESI): 439.2 [M+1].

### Step 3

### (S)-4-Ethyl-8-fluoro-4-hydroxy-10-(3-hydroxypropyl)-9-methyl-3,14-dioxo-3,4,12,14-tetrahydro-1H-pyrano[3′,4′:6,7]indolizino[1,2-b]quinoline 6-oxide 22e

22d (334 mg, 684 μmol) was dissolved in 30 mL of acetic acid, added with hydrogen peroxide (12.8 g, 113 mmol, the content is 30%), and stirred at 70° C. for 1.5 hours. The completion of the reaction was monitored by LC-MS. The reaction solution was cooled to 0° C., added with 15 mL of sodium thiosulfate solution to quench the reaction, and extracted with ethyl acetate (20 mL×2), the organic phase was washed with saturated sodium chloride solution (15 mL×2), the filtrate was dried over anhydrous sodium sulfate and filtered, and the filtrate was concentrated under reduced pressure. The residue was purified by silica gel column chromatography using the eluent system A to obtain the title product 22e (110 mg, yield: 31%) in the form of yellow solid.

MS m/z (ESI): 455.1 [M+1].

### Step 4

### (S)-3-(11-Chloro-4-ethyl-8-fluoro-4-hydroxy-9-methyl-3,14-dioxo-3,4,12,14-tetrahydro-1H-pyrano[3′,4′:6,7]indolizino[1,2-b]quinolin-10-yl)propyl formate 22f

22e (110 mg, 227 μmol) was dissolved in 5 mL of N,N-dimethylformamide, added with oxalyl chloride (144 mg, 1.13 mmol), and stirred at 0° C. for 1 hour. The reaction solution was added with 8 mL of water, extracted with ethyl acetate (10 mL×3), the organic phase was washed with saturated sodium chloride solution (8 mL×3), the filtrate was dried over anhydrous sodium sulfate and filtered, and the filtrate was concentrated under reduced pressure to obtain the crude title product 22f (100 mg) in the form of yellow solid. The product was used directly in the next reaction without purification.

MS m/z (ESI): 501.1 [M+1].

### Step 5

### (S)-11-Chloro-4-ethyl-8-fluoro-4-hydroxy-10-(3-hydroxypropyl)-9-methyl-1,12-dihydro-14H-pyrano[3′,4′:6,7]indolizino[1,2-b]quinoline-3,14(4H)-dione 22 g

22f (100 mg, 199 μmol) was dissolved in 4 mL of methanol, added with aqueous hydrochloric acid solution (2 M, 1.00 mL), and stirred at 25° C. for 1 hour. Methanol was removed by concentration under reduced pressure, 3 mL of water was added to the residue, and ethyl acetate (5 mL×3) was used for extraction. The organic phase was washed with saturated sodium chloride solution (8 mL×3), the filtrate was dried over anhydrous sodium sulfate and filtered, and the filtrate was concentrated under reduced pressure to obtain the crude title product 22 g (90 mg) in the form of yellow solid. The product was used directly in the next reaction without purification.

MS m/z (ESI): 473.1 [M+1].

### Step 6

### (S)-3-(11-Chloro-4-ethyl-8-fluoro-4-hydroxy-9-methyl-3,14-dioxo-3,4,12,14-tetrahydro-1H-pyrano[3′,4′:6,7]indolizino[1,2-b]quinolin-10-yl)propanal 22h

22 g (90.0 mg, 190 μmol) was dissolved in 4 mL of dichloromethane, added with Dess-Martin reagent (242 mg, 570 μmol), and stirred at 25° C. for 2 hours. 5 mL of water was added to the reaction solution, and dichloromethane (3 mL×2) was used for extraction. The filtrate was dried over anhydrous sodium sulfate and filtered, and the filtrate was concentrated under reduced pressure to obtain the crude title product 22h (90 mg) in the form of yellow solid. The product was used directly in the next reaction without purification.

MS m/z (ESI): 471.1 [M+1].

### Step 7

### tert-Butyl (S)-(10-ethyl-6-fluoro-10-hydroxy-5-methyl-11,14-dioxo-3,4,10,11,14,16-hexahydro-13H-azepino[2,3,4-de]pyrano[3′,4′:6,7]indolizino[1,2-b]quinolin-1(2H)-yl)carbamate 22i

22h (90.0 mg, 191 μmol) and tert-butyl carbazate (27.8 mg, 210 μmol) were dissolved in 1 mL of methanol, added with sodium cyanoborohydride (60.1 mg, 955 μmol), and stirred at 25° C. for 4 hours. The methanol was removed by concentration under reduced pressure, 2 mL of water was added to the residue, and dichloromethane (3 mL×2) was used for extraction. The filtrate was dried over anhydrous sodium sulfate and filtered, and the filtrate was concentrated under reduced pressure. 15 mg of the residue was taken and purified by pre-HPLC (separation conditions: chromatographic column: Phenomenex luna C18, 150×25 mm×10 μm, mobile phase: A-water (0.2% formic acid), B-acetonitrile, gradient elution, B %: 33%-63%) to obtain the title product 22i (1.01 mg, yield: 13%) in the form of off-white solid.

MS m/z (ESI): 551.4 [M+1].

### Step 8

### (S)-1-Amino-10-ethyl-6-fluoro-10-hydroxy-5-methyl-2,3,4,10,13,16-hexahydro-14H-azepino[2,3,4-de]pyrano[3′,4′:6,7]indolizino[1,2-b]quinoline-11,14(1H)-dione 22j

22i (20.0 mg, 22.3 μmol) was dissolved in 1 mL of dichloromethane, added with trifluoroacetic acid (2.05 g, 17.9 mmol), and stirred at 25° C. for 1 hour. The methanol was removed by concentration under reduced pressure, 2 mL of water was added to the residue, and the mixture was extracted with dichloromethane (3 mL×2). The filtrate was dried over anhydrous sodium sulfate and filtered, and the filtrate was concentrated under reduced pressure. 6 mg of the residue was purified by pre-HPLC (separation conditions: chromatographic column: Welch Xtimate C18, 150×25 mm×5 μm, mobile phase: A-water (0.2% formic acid), B-acetonitrile, gradient elution, B %: 8%-38%) to obtain the title product 22j (1.06 mg, yield: 64%) in the form of yellow colloid.

MS m/z (ESI): 451.4 [M+1].

### Step 9

### (S)-2-(Benzyloxy)-N-(10-ethyl-6-fluoro-10-hydroxy-5-methyl-11,14-dioxo-3,4,10,11,14,16-hexahydro-13H-azepino[2,3,4-de]pyrano[3′,4′:6,7]indolizino[1,2-b]quinolin-1(2H)-yl)acetamide 22k

22j (20 mg, 25.44 μmol) was dissolved in 0.5 mL of tetrahydrofuran and 0.5 mL of saturated sodium bicarbonate solution, added with 2-benzyloxyacetyl chloride (23.48 mg, 127.18 μmol) under nitrogen protection, and stirred at 25° C. for 10 hours under nitrogen protection. The reaction solution was added with 1 mL of water and extracted with ethyl acetate (2 mL×3). The organic phase was washed with saturated sodium chloride solution (2 mL×2), dried over anhydrous sodium sulfate, and filtered. The filtrate was concentrated under reduced pressure. The residue was purified by preparative thin layer chromatography using the development system A to obtain the title product 22k (4 mg, yield: 22%) in the form of yellow solid.

MS m/z (ESI): 599.1 [M+1].

### Step 10

### (S)-N-(10-Ethyl-6-fluoro-10-hydroxy-5-methyl-11,14-dioxo-3,4,10,11,14,16-hexahydro-13H-azepino[2,3,4-de]pyrano[3′,4′:6,7]indolizino[1,2-b]quinolin-1(2H)-yl)-2-hydroxyacetamide 22

22k (3.5 mg, 4.85 μmol) was dissolved in 1 mL of tetrahydrofuran, added with palladium(II) chloride (860.53 μg, 4.85 μmol), and subjected to replacement with hydrogen three times. The reaction solution was stirred at 0° C. under hydrogen gas (15 Psi) for 0.5 h. The reaction solution was filtered with Celite, the filtrate was concentrated by vacuum distillation, and the residue was purified by pre-HPLC (separation conditions: chromatographic column: Phenomenex luna C18, 150×25 mm×10 μm, mobile phase: A-water (0.2% formic acid), B-acetonitrile, gradient elution, B %: 13%-43%) to obtain the title product 22 (0.3 mg, yield: 9%) in the form of yellow solid.

MS m/z (ESI): 509.3 [M+1].

### Example 2-24: Preparation of Compound 24

### (S)-N-((8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxyacetamide 24

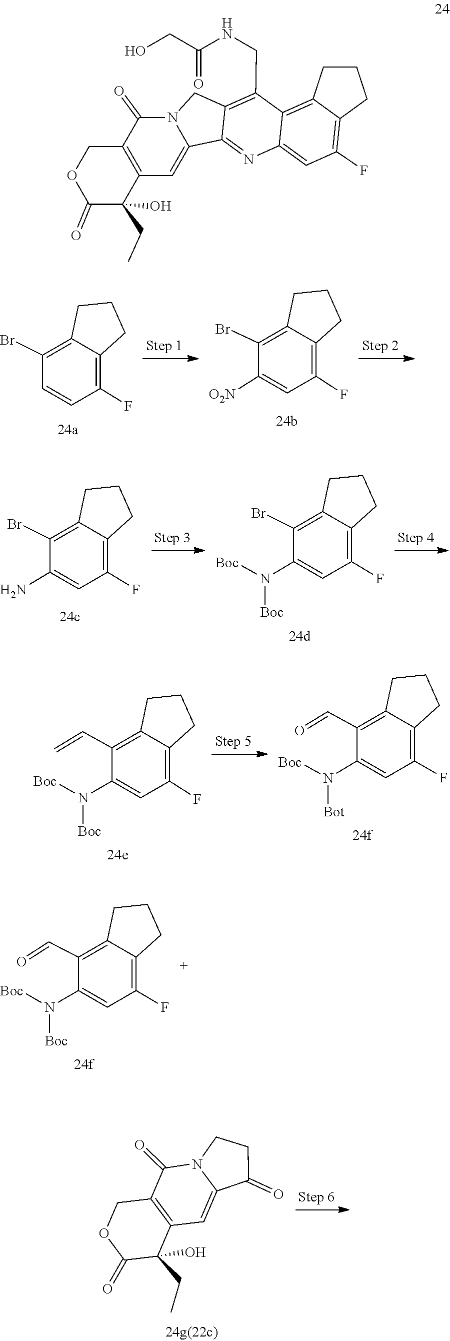

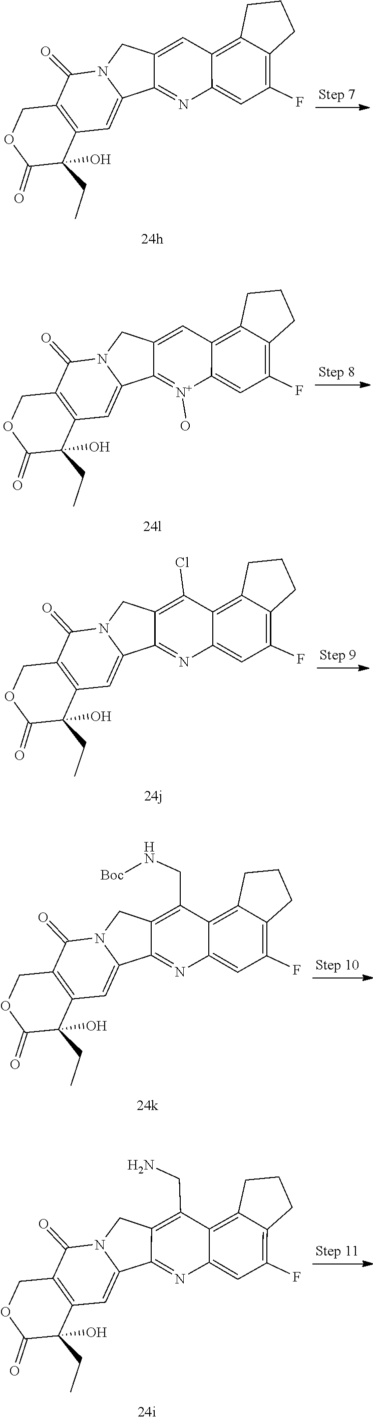

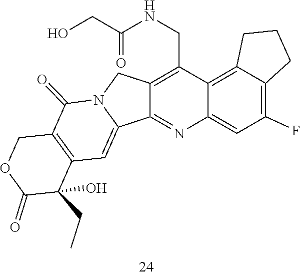 [SMILES: CC[C@@]1(O)C(=O)OCc2c1cc1n(c2=O)Cc2c-1nc1cc(F)c3c(c1c2CNC(=O)CO)CCC3]

### Step 1

### 4-Bromo-7-fluoro-5-nitro-2,3-dihydro-1H-indene 24b

24a (17 g, 79.05 mmol, prepared by applying the method disclosed in Example 9 on page 31 of patent application “WO2021093820 A1”) was dissolved in 200 mL of trifluoroacetic acid under nitrogen protection, and cooled to 0° C. in ice-water bath. To the reaction solution, nitric acid (20.45 g, 292.08 mmol, the content is 90%) was added, and stirred at 0° C. for 10 minutes; then the ice-water bath was removed, and the stirring was continued at 25° C. for 30 minutes. 100 mL of water was added, and the diluted reaction solution was extracted with ethyl acetate (500 mL×3). The organic phase was washed with water (500 mL×3), dried over anhydrous sodium sulfate, and filtered. The filtrate was concentrated by vacuum distillation. The residue was purified by silica gel column chromatography with the developing system B to obtain the title product 24b (8.96 g, yield: 44%) in the form of colorless oil.

¹H NMR (400 MHz, CDCl₃) δ 7.45 (d, J=8.0 Hz, 1H), 3.05-3.16 (m, 4H), 2.18-2.27 (m, 2H).

### Step 2

### 4-Bromo-7-fluoro-2,3-dihydro-1H-inden-5-amine 24c

24b (8.96 g, 34.61 mmol) was dissolved in a mixed solution of 100 mL of ethanol and 20 mL of water, and added with iron powder (5.80 g, 103.82 mmol) and ammonium chloride (5.55 g, 103.82 mmol) in sequence under nitrogen protection, and the reaction solution was stirred at 80° C. for 1 hour. The reaction solution was filtered, and the filtrate was concentrated by vacuum distillation to obtain the crude title product 24c (7.2 g) in the form of white solid. The product was used directly in the next step without purification.

MS m/z (ESI): 229.9 [M+1].

### Step 3

### tert-Butyl (4-bromo-7-fluoro-2,3-dihydro-1H-inden-5-yl)(tert-butoxycarbonyl)carbamate 24d

24c (7.2 g, 31.29 mmol) was dissolved in 50 mL of tetrahydrofuran, added with di-tert-butyl dicarbonate (27.32 g, 125.18 mmol), 4-dimethylaminopyridine (38.23 mg, 312.94 μmol) and triethylamine (12.67 g, 125.18 mmol), and the reaction solution was stirred at 25° C. for 16 hours. The reaction solution was concentrated by vacuum distillation, and the obtained residue was purified by silica gel column chromatography with the developing system B to obtain the title product 24d (9.2 g, yield: 68%) in the form of white solid.

MS m/z (ESI): 317.9 [M+1-56-56].

### Step 4

### tert-Butyl (tert-butyloxycarbonyl)(7-fluoro-4-vinyl-2,3-dihydro-1H-inden-5-yl)carbamate 24e

24d (1.5 g, 4.38 mmol) and pinacol vinylboronate (810.20 mg, 5.26 mmol) were dissolved in a mixed solution of 6 mL of 1,4-dioxane and 2 mL of water, and added with tripotassium phosphate (2.79 g, 13.15 mmol) and (2-dicyclohexylphosphino-2′,4′,6′-tri-isopropyl-1,1′-biphenyl)(2′-amino-1,1′-biphenyl-2-yl)palladium (II) methanesulfonate (371.07 mg, 438.38 μmol) in sequence under nitrogen protection, and the reaction solution was stirred at 95° C. for 10 hours. 20 mL of water was added, and the diluted reaction solution was extracted with ethyl acetate (10 mL×3). The organic phase was washed with saturated sodium chloride solution (5 mL×2), dried over anhydrous sodium sulfate, and filtered. The filtrate was concentrated by vacuum distillation. The obtained residue was purified by silica gel column chromatography using the developing system B to obtain the title product 24e (350 mg, yield: 26%) in the form of colorless oil.

MS m/z (ESI): 222.1 [M+1-100-56].

### Step 5

### tert-Butyl (tert-butyloxycarbonyl)(7-fluoro-4-formyl-2,3-dihydro-1H-inden-5-yl)carbamate 24f

24e (755 mg, 2.00 mmol) was dissolved in a mixed solution of 1.5 mL of dioxane and 1.5 mL of water, added with sodium periodate (1.29 g, 6.01 mmol) and potassium osmate dihydrate (73.8 mg, 200 μmol), and stirred at 25° C. for 5 hours. The reaction solution was filtered, and the filtrate was concentrated by vacuum distillation. The obtained residue was purified by silica gel column chromatography using the developing system B to obtain the title product 24f (538 mg, yield: 71%) in the form of yellow solid.

MS m/z (ESI): 180.2 [M+1-100-100].

### Step 6

### (S)-8-Ethyl-4-fluoro-8-hydroxy-1,2,3,8,11,14-hexahydro-9H,12H-cyclopenta[f]pyrano[3′,4′:6,7]indolizino[1,2-b]quinoline-9,12-dione 24h

24f (538 mg, 1.42 mmol) and 24 g (i.e., the above-mentioned Compound 22c) (375 mg, 1.42 mmol, prepared by applying the method disclosed in Example 30 on page 28 of patent application “WO2019238046 A1”) were dissolved in 10 mL of ethanol, added with concentrated hydrochloric acid (12 M, 1.58 mL), and stirred at 80° C. for 2 hours. The reaction solution was concentrated by vacuum distillation to remove ethanol, 10 mL of water was added, and the filter cake was obtained by filtration, and the crude title product 24h (500 mg) was obtained as a yellow solid. The product was used directly in the next step without purification.

MS m/z (ESI): 407.2 [M+1].

### Step 7

### (S)-8-Ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinoline 6-oxide 24i

24h (500 mg, 1.21 mmol) was dissolved in 70 mL of acetic acid, added with hydrogen peroxide (16.9 g, 149 mmol, the content is 30%) under nitrogen protection, and stirred at 75° C. for 3 hours. LCMS showed that the starting material 24 h was in excess, hydrogen peroxide (16.5 g, 145 mmol, the content is 30%) was added again, the resulting mixture was stirred at 75° C. for 4 hours. The reaction solution was poured into 100 mL of water, and filtered to obtain a filter cake to obtain the crude title product 24i (400 mg) as a yellow solid. The product was used directly in the next step without purification.

MS m/z (ESI): 423.2 [M+1].

### Step 8

### (S)-15-Chloro-8-ethyl-4-fluoro-8-hydroxy-1,2,3,8,11,14-hexahydro-9H,12H-cyclopentaf pyrano[3′,4′:6,7]indolizino[1,2-b]quinoline-9,12-dione 24j

24i (100 mg, 142 μmol) was dissolved in 2 mL of N,N-dimethylformamide, cooled to 0° C. in ice-water bath under nitrogen protection, added with oxalyl chloride (45.0 mg, 355 μmol), and stirred at 25° C. for 1 hour. The reaction solution was poured into 10 mL of water and filtered to obtain a filter cake, and the crude title product 24j (70 mg) was obtained as a brown solid. The product was used directly in the next step without purification.

MS m/z (ESI): 441.2 [M+1].

### Step 9

### tert-Butyl (S)-((8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)carbamate 24k

24j (20.0 mg, 26.3 μmol) and potassium (((tert-butoxycarbonyl)amino)methyl)trifluoroborate (14.0 mg, 59.2 μmol) were dissolved in a mixed solution of 1 mL of dioxane and 0.2 mL of water, added with 1,1-bis(diphenylphosphino)ferrocenepalladium dichloride (2.89 mg, 3.95 μmol) and potassium carbonate (25.1 mg, 118 μmol) under nitrogen protection, and stirred at 95° C. for 1 hour under nitrogen protection. The reaction mixture was poured into aqueous hydrochloric acid solution (1 M, 2 mL), and the filtrate was concentrated by vacuum distillation to obtain the crude title product 24k (20 mg) as a brown oil. The product was used directly in the next step without purification.

MS m/z (ESI): 436.2 [M+1-100].

### Step 10

### (S)-15-(Aminomethyl)-8-ethyl-4-fluoro-8-hydroxy-1,2,3,8,11,14-hexahydro-9H,12H-cyclopenta[f]pyrano[3′,4′:6,7]indolizino[1,2-b]quinoline-9,12-dione 241

24k (1.00 mg, 1.87 μmol) was dissolved in 0.6 mL of dichloromethane, added with a dioxane solution of hydrochloric acid (4 M, 0.2 mL), and stirred at 25° C. for 1 hour. 1 mL of water was added, and the diluted reaction solution was extracted with ethyl acetate (1 mL×3). The aqueous phase was filtered, and the filtrate was concentrated by vacuum distillation to obtain the crude title product 24l (0.5 mg) as a yellow solid. The product was used directly in the next reaction without purification.

MS m/z (ESI): 436.1 [M+1].

### Step 11

### (S)-N-((8-Ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxyacetamide 24

24l (13.3 mg, 22.9 μmol) and 2-hydroxyacetic acid (2.10 mg, 27.5 μmol) were dissolved in 1 mL of dichloromethane, added with N,N-diisopropylethylamine (8.90 mg, 68.9 μmol), 1-(3-dimethylaminopropyl)-3-ethylcarbodiimide hydrochloride (6.60 mg, 34.4 μmol) and 1-hydroxybenzotriazole (4.65 mg, 34.4 μmol), and stirred at 25° C. for 30 minutes. The reaction solution was concentrated by vacuum distillation, added with 2 mL of water, and extracted with ethyl acetate (1 mL×3), the organic phase was washed with saturated sodium chloride solution (1 mL), the organic phase was dried over anhydrous sodium sulfate and filtered, and the filtrate was concentrated by vacuum distillation. The residue was purified by preparative HPLC (separation conditions: chromatographic column: Welch ultimate C18 150×25 mm×7 μm, mobile phase: A-water (containing 0.225% formic acid), B-acetonitrile, gradient elution, flow rate: 25 mL/min, instrument: GILSON GX-281) to obtain the title product 24 (0.98 mg, yield: 8%) as a yellow solid.

MS m/z (ESI): 494.1 [M+1].

¹H NMR (400 MHz, CD₃OD) δ 7.71-7.66 (m, 1H), 7.63 (s, 1H), 5.61-5.56 (m, 1H), 5.50-5.48 (m, 1H), 5.43-5.35 (m, 3H), 5.18-5.09 (m, 3H), 3.73-3.65 (m, 2H), 3.20-3.12 (m, 2H), 2.39-2.29 (m, 2H), 2.02-1.93 (m, 2H), 1.05-0.94 (m, 3H).

### Examples 2-25 to 2-33: Preparation of Compounds 25 to 33

By referring to Example 2-24, corresponding substrates were used to prepare the following compounds 25 to 33:

### Example 2-25

### (S)-N-(((S)-8-Ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxypropanamide 25

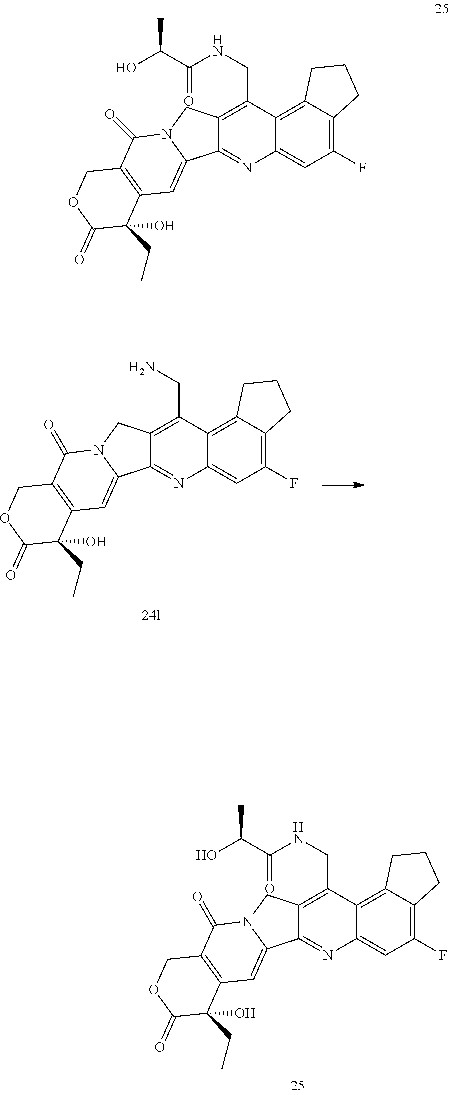 [SMILES: CC[C@@]1(O)C(=O)OCc2c1cc1n(c2=O)Cc2c-1nc1cc(F)c3c(c1c2CNC(=O)[C@H](C)O)CCC3 ; CC[C@@]1(O)C(=O)OCc2c1cc1n(c2=O)Cc2c-1nc1cc(F)c3c(c1c2CN)CCC3 ; CC[C@@]1(O)C(=O)OCc2c1cc1n(c2=O)Cc2c-1nc1cc(F)c3c(c1c2CNC(=O)[C@H](C)O)CCC3]

24l (22.3 mg, 45.1 μmol) and (2R)-2-hydroxypropionic acid (4.07 mg, 45.2 μmol) were dissolved in 0.5 mL of dichloromethane, added with N,N-diisopropylethylamine (17.5 mg, 135 μmol), 1-(3-dimethylaminopropyl)-3-ethylcarbodiimide hydrochloride (10.4 mg, 54.2 μmol) and 1-hydroxybenzotriazole (7.33 mg, 54.2 μmol), and stirred at 25° C. for 3 hours. The reaction solution was concentrated by vacuum distillation, and the residue was purified by pre-HPLC (separation conditions: chromatographic column: Phenomenex luna C18 250×50 mm×15 μm, mobile phase: A-water (0.225% formic acid), B-acetonitrile, gradient elution, B %: 23%-53%) to obtain the title product 25 (0.49 mg, yield: 2%) as a yellow gum. SFC analysis: retention time: 0.931 minutes, purity: 97%. (Chromatographic column: Chiralcel OD-3 50×4.6 mm I.D., 3 μm, mobile phase: A-carbon dioxide, B-isopropanol and acetonitrile (0.05% diethylamine), isocratic elution: B: 40%, flow rate: 3 mL/min, instrument: Shimadzu LC-30ADsf).

MS m/z (ESI): 508.1 [M+1].

¹H NMR (400 MHz, CD₃OD) δ 7.70 (d, J=9.6 Hz, 1H), 7.63 (s, 1H), 5.63-5.56 (m, 1H), 5.43-5.36 (m, 3H), 5.15-5.10 (m, 2H), 4.25-4.18 (m, 1H), 3.71-3.66 (m, 2H), 3.19-3.13 (m, 2H), 2.39-2.31 (m, 2H), 2.01-1.93 (m, 2H), 1.43-1.38 (m, 3H), 1.04-0.98 (m, 3H).

### Example 2-26

### (R)-N-(((S)-8-Ethyl-4-fluoro-8-hydroxy-9,12-dioxo-2,3,8,9,12,14-hexahydro-1H,11H-cyclo penta[f]pyrano[3′,4′:6,7]indolizino[1,2-b]quinolin-15-yl)methyl)-2-hydroxypropanamide 26

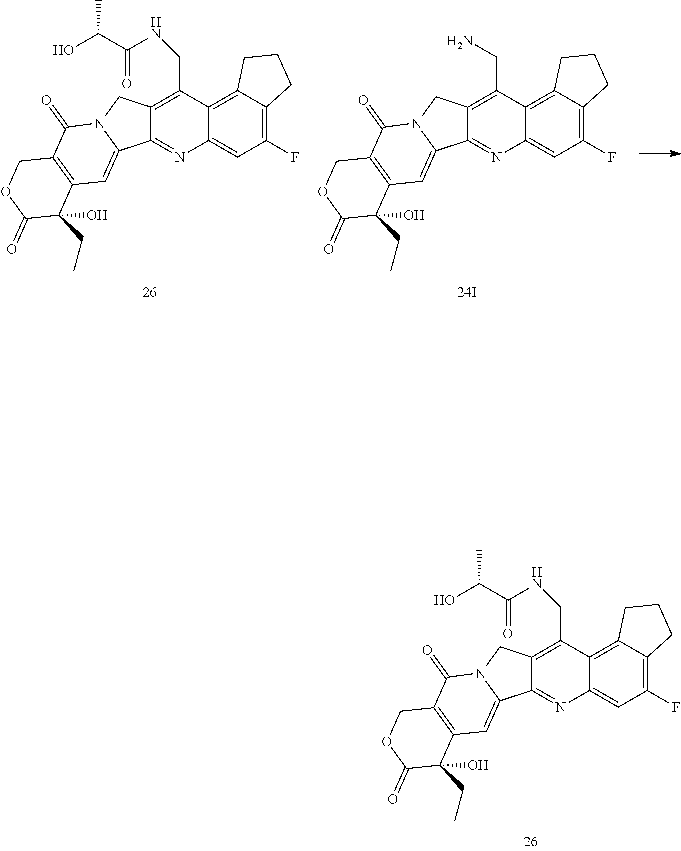

24l (22.3 mg, 45.2 μmol) and (2S)-2-hydroxypropionic acid (4.07 mg, 45.1 μmol) were dissolved in 0.5 mL of dichloromethane, added with N,N-diisopropylethylamine (17.5 mg, 135 μmol), 1-(3-dimethylaminopropyl)-3-ethylcarbodiimide hydrochloride (10.4 mg, 54.2 μmol) and 1-hydroxybenzotriazole (7.33 mg, 54.2 μmol), and stirred at 25° C. for 3 hours. The reaction solution was concentrated by vacuum distillation, and the residue was purified by pre-HPLC (separation conditions: chromatographic column: Welch ultimate C18 150×25 mm×7 μm, mobile phase: A-water (0.225% formic acid), B-acetonitrile, gradient elution, B %: 20%-50%) to obtain the title product 26 (1.09 mg, yield: 5%) as a yellow jelly. SFC analysis: retention time: 0.745 minutes, purity: 98%. (Chromatographic column: Chiralcel OD-3 50×4.6 mm I.D., 3 μm, mobile phase: A-carbon dioxide, B-isopropanol and acetonitrile (0.05% diethylamine), isocratic elution: B: 40%, flow rate: 3 mL/min, instrument: Shimadzu LC-30ADsf).

MS m/z (ESI): 508.1 [M+1].

¹H NMR (400 MHz, CD₃OD) δ 7.72 (d, J=9.4 Hz, 1H), 7.66 (s, 1H), 5.63-5.59 (m, 1H), 5.45-5.39 (m, 3H), 5.17-5.14 (m, 2H), 4.26-4.22 (m, 1H), 3.74-3.68 (m, 2H), 3.21-3.16 (m, 2H), 2.40-2.35 (m, 2H), 2.01-1.95 (m, 2H), 1.44-1.39 (m, 3H), 1.06-1.00 (m, 3H).

### Example 2-27

### (S)-2-Cyclopropyl-N-(((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxyacetamide 27

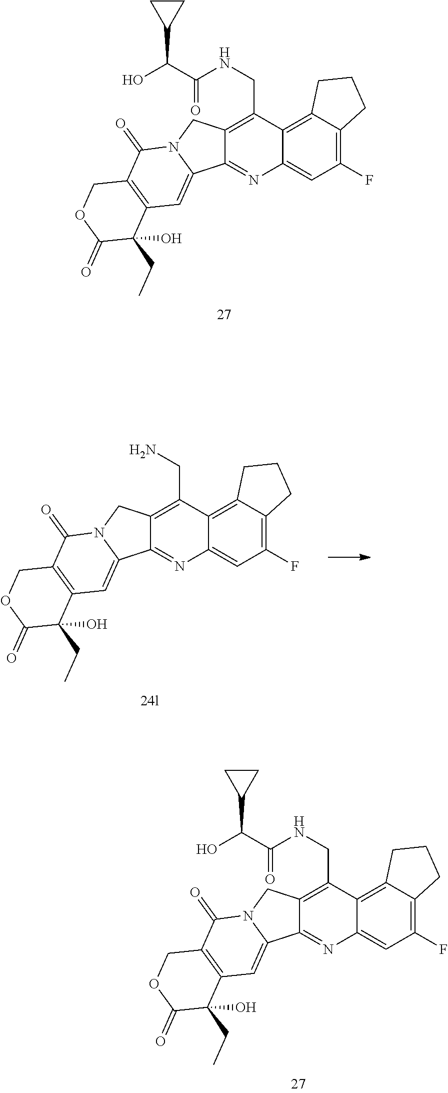 [SMILES: CC[C@@]1(O)C(=O)OCc2c1cc1n(c2=O)Cc2c-1nc1cc(F)c3c(c1c2CNC(=O)[C@@H](O)C1CC1)CCC3 ; CC[C@@]1(O)C(=O)OCc2c1cc1n(c2=O)Cc2c-1nc1cc(F)c3c(c1c2CN)CCC3 ; CC[C@@]1(O)C(=O)OCc2c1cc1n(c2=O)Cc2c-1nc1cc(F)c3c(c1c2CNC(=O)[C@@H](O)C1CC1)CCC3]

24l (25.0 mg, 50.5 μmol) and (2S)-2-cyclopropyl-2-hydroxyacetic acid (8.80 mg, 75.8 μmol) were dissolved in 1 mL of dichloromethane, added with N,N-diisopropylethylamine (26.1 mg, 202 μmol), 1-(3-dimethylaminopropyl)-3-ethylcarbodiimide hydrochloride (14.5 mg, 75.8 μmol) and 1-hydroxybenzotriazole (10.2 mg, 75.8 μmol), and stirred at 25° C. for 30 minutes. The reaction solution was concentrated by vacuum distillation, and the residue was purified by pre-HPLC (separation conditions: chromatographic column: Welch ultimate C18 150×25 mm×7 μm, mobile phase: A-water (0.225% formic acid), B-acetonitrile, gradient elution, flow rate: 25 mL/min) to obtain the title product 27 (2.15 mg, yield: 8%) as a brown oil. SFC analysis: retention time: 1.131 minutes, purity: 97%. (Chromatographic column: Chiralcel OD-3 50×4.6 mm I.D., 3 μm; mobile phase: A-carbon dioxide: B-ethanol (0.05% diethylamine), isocratic elution: B: 40%, flow rate: 3 mL/min, instrument: Shimadzu LC-30ADsf).

MS m/z (ESI): 534.3 [M+1]. ¹H NMR (400 MHz, CD₃OD) δ 7.72-7.66 (m, 1H), 7.63 (s, 1H), 5.61-5.55 (m, 1H), 5.45-5.34 (m, 3H), 5.12 (s, 2H), 3.75-3.64 (m, 3H), 3.19-3.10 (m, 2H), 2.40-2.30 (m, 2H), 2.02-1.91 (m, 2H), 1.21-1.14 (m, 1H), 1.06-0.93 (m, 3H), 0.57-0.40 (m, 4H).

### Example 2-28

### (R)-2-Cyclopropyl-N-(((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxyacetamide 28

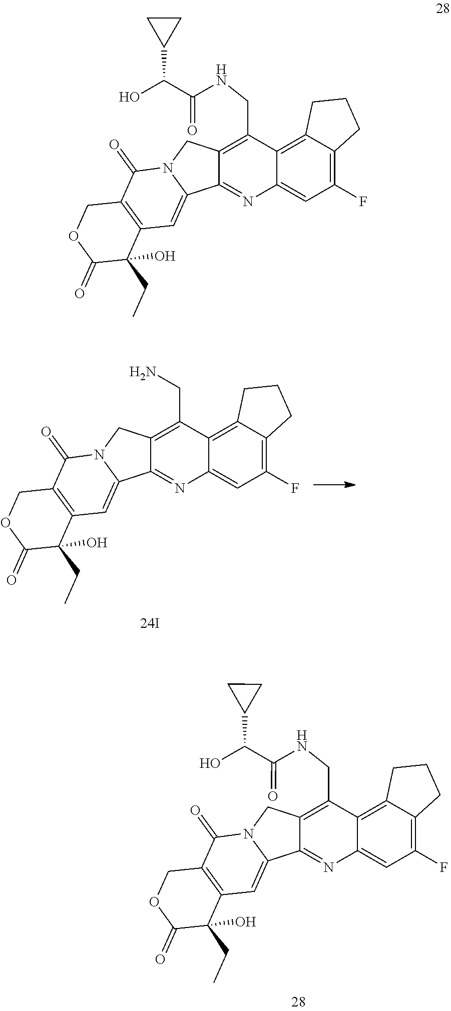 [SMILES: CC[C@@]1(O)C(=O)OCc2c1cc1n(c2=O)Cc2c-1nc1cc(F)c3c(c1c2CNC(=O)[C@H](O)C1CC1)CCC3 ; CC[C@@]1(O)C(=O)OCc2c1cc1n(c2=O)Cc2c-1nc1cc(F)c3c(c1c2CN)CCC3 ; CC[C@@]1(O)C(=O)OCc2c1cc1n(c2=O)Cc2c-1nc1cc(F)c3c(c1c2CNC(=O)[C@H](O)C1CC1)CCC3]

24l (25.0 mg, 50.5 μmol) and (2R)-2-cyclopropyl-2-hydroxyacetic acid (8.80 mg, 75.8 μmol) were dissolved in 1 mL of dichloromethane, added with N,N-diisopropylethylamine (26.1 mg, 202 μmol), 1-(3-dimethylaminopropyl)3-ethylcarbodiimide hydrochloride (14.5 mg, 75.8 μmol) and 1-hydroxybenzotriazole (10.2 mg, 75.8 μmol), and stirred at 25° C. for 30 minutes. The reaction mixture was concentrated by vacuum distillation, and the residue was purified by pre-HPLC (separation conditions: column: Phenomenex luna C18 150×25 mm×10 μm, mobile phase: A-water (0.225% formic acid), B-acetonitrile, gradient elution) to obtain the title product 28 (2.77 mg, yield: 10%) as a brown oil. SFC analysis: retention time: 0.898 minutes, purity: 99%. (Chromatographic column: Chiralcel OD-3 50×4.6 mm I.D., 3 μm; mobile phase: A-carbon dioxide: B-ethanol (0.05% diethylamine), isocratic elution: B: 40%, flow rate: 3 m/min, instrument: Shimadzu LC-30ADsf).

MS m/z (ESI): 534.4 [M+1].

¹H NMR (400 MHz, CD₃OD) δ 7.75-7.71 (m, 1H), 7.66 (s, 1H), 5.63-5.59 (m, 1H), 5.47-5.43 (m, 2H), 5.40-5.37 (m, 1H), 5.16-5.14 (m, 2H), 3.75-3.66 (m, 3H), 3.21-3.15 (m, 2H), 2.41-2.32 (m, 2H), 2.04-1.95 (m, 2H), 1.21-1.15 (m, 1H), 1.07-1.00 (m, 3H), 0.56-0.42 (m, 4H).

### Examples 2-29 to 2-33

By referring to Example 2-28, corresponding substrates were used to prepare the following compounds:

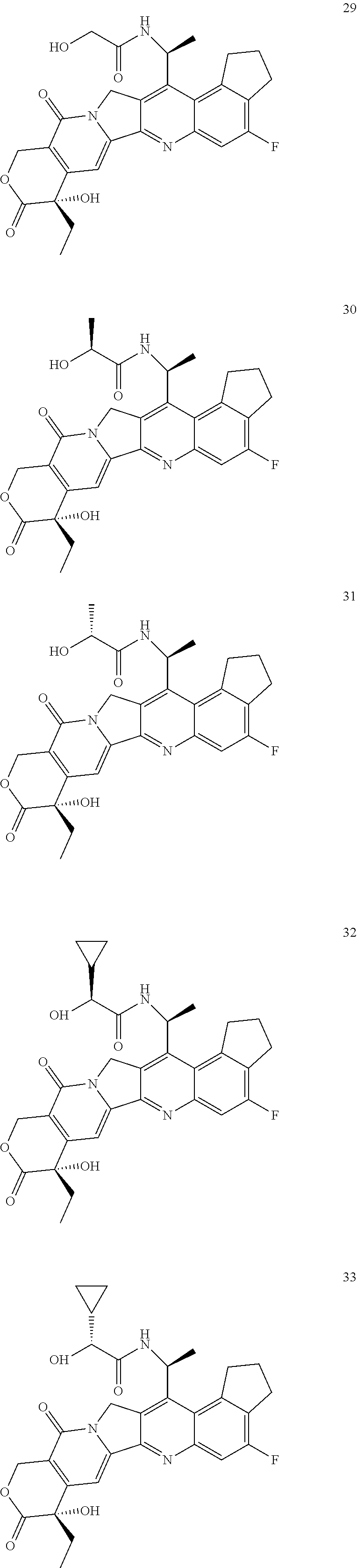 [SMILES: CC[C@@]1(O)C(=O)OCc2c1cc1n(c2=O)Cc2c-1nc1cc(F)c3c(c1c2[C@H](C)NC(=O)CO)CCC3 ; CC[C@@]1(O)C(=O)OCc2c1cc1n(c2=O)Cc2c-1nc1cc(F)c3c(c1c2[C@H](C)NC(=O)[C@H](C)O)CCC3 ; CC[C@@]1(O)C(=O)OCc2c1cc1n(c2=O)Cc2c-1nc1cc(F)c3c(c1c2[C@H](C)NC(=O)[C@@H](C)O)CCC3 ; CC[C@@]1(O)C(=O)OCc2c1cc1n(c2=O)Cc2c-1nc1cc(F)c3c(c1c2[C@H](C)NC(=O)[C@@H](O)C1CC1)CCC3 ; CC[C@@]1(O)C(=O)OCc2c1cc1n(c2=O)Cc2c-1nc1cc(F)c3c(c1c2[C@H](C)NC(=O)[C@H](O)C1CC1)CCC3]

### Example 2-34

### (S)-N-((8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)methyl)-2-hydroxyacetamide 34

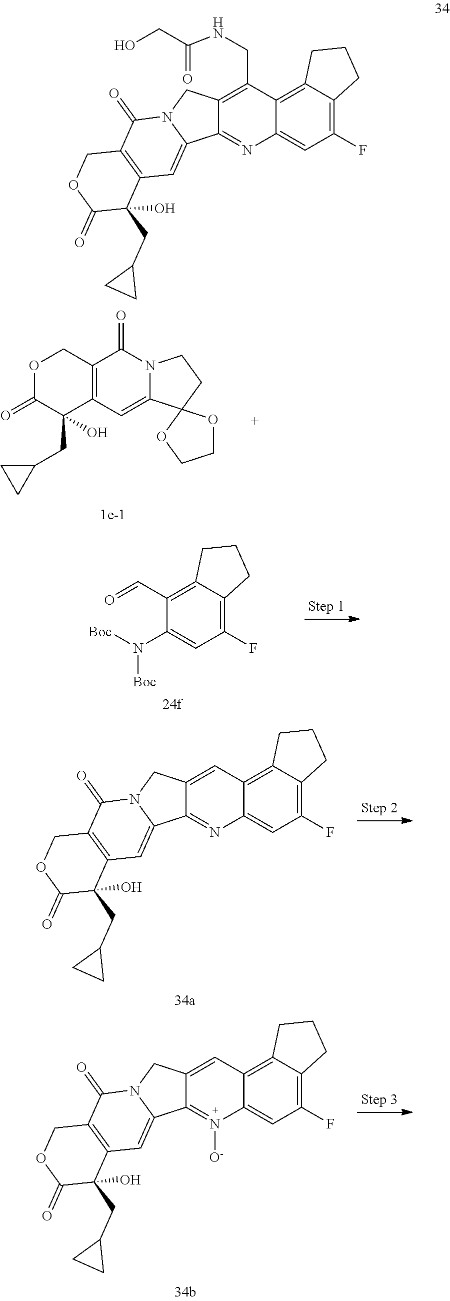

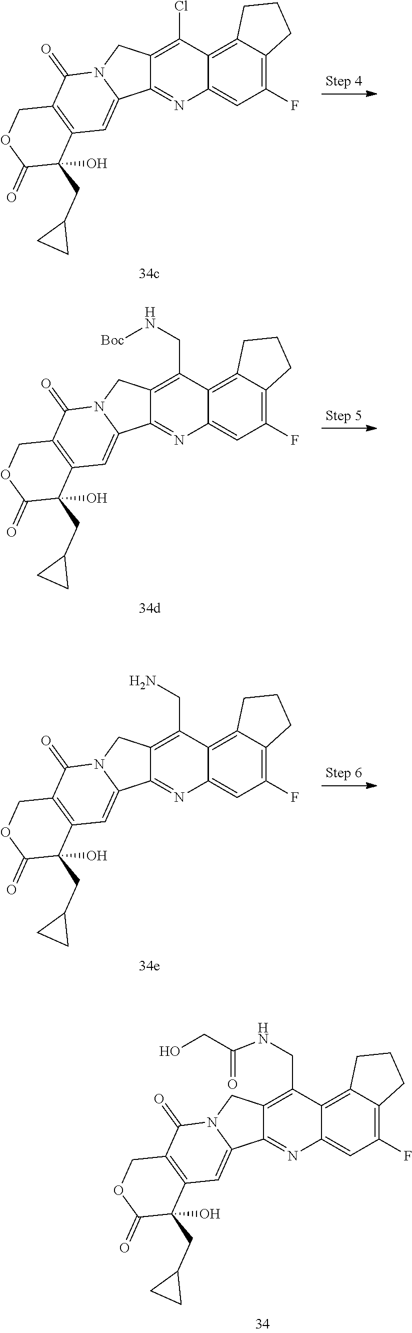 [SMILES: O=C1OCc2c(cc3n(c2=O)Cc2c-3nc3cc(F)c4c(c3c2Cl)CCC4)[C@@]1(O)CC1CC1 ; CC(C)(C)OC(=O)NCc1c2c(nc3cc(F)c4c(c13)CCC4)-c1cc3c(c(=O)n1C2)COC(=O)[C@]3(O)CC1CC1 ; NCc1c2c(nc3cc(F)c4c(c13)CCC4)-c1cc3c(c(=O)n1C2)COC(=O)[C@]3(O)CC1CC1 ; O=C(CO)NCc1c2c(nc3cc(F)c4c(c13)CCC4)-c1cc3c(c(=O)n1C2)COC(=O)[C@]3(O)CC1CC1]

### Step 1

24f (500 mg, 1.32 mmol) and 1e-1 (578 mg, 1.73 mmol) were dissolved in 20 mL of ethanol, added with concentrated hydrochloric acid (12 M, 5.02 mL), and stirred at 80° C. for 16 hours. The reaction solution was concentrated by vacuum distillation, 10 mL of water was added to precipitate solid, and the solid was collected by filtration to obtain the title product 34a (496 mg) as a yellow solid. The product was used directly in the next step without purification.

MS m/z (ESI): 433.1 [M+1].

### Step 2

34a (596 mg, 1.38 mmol) was dissolved in 36 mL of acetic acid, added with hydrogen peroxide (3.86 g, 34.0 mmol, the content is 30%), and stirred at 75° C. for 3 hours. 100 mL of water was added to the reaction solution to precipitate solid, and the solid was collected by filtration to obtain the title product 34b (500 mg) as a yellow solid. The product was used directly in the next step without purification.

MS m/z (ESI): 449.0 [M+1].

### Step 3

34b (490 mg, 915 μmol) was dissolved in 30 mL of N,N-dimethylformamide, cooled to 0° C. in ice-water bath under nitrogen protection, added with oxalyl chloride (465 mg, 3.66 mmol), and stirred at 0° C. for 1 hour. The reaction solution was poured into 100 mL of water to precipitate solid, and the solid was collected by filtration to obtain the title product 34c (500 mg) as a yellow solid. The product was used directly in the next reaction without purification.

MS m/z (ESI): 467.1 [M+1].

### Step 4

34c (200 mg, 428 μmol) was dissolved in a mixed solvent of 5 mL of dioxane and 0.5 mL of water, added with potassium (((tert-butoxycarbonyl)amino)methyl)trifluoroborate (203 mg, 856 μmol), 1,1′-bis(diphenylphosphino)ferrocene palladium(II) dichloride (31.3 mg, 42.8 μmol) and tripotassium phosphate (272 mg, 1.29 mmol), and stirred at 95° C. for 5 hours under nitrogen protection. The reaction solution was concentrated by vacuum distillation to obtain the title product 34d (250 mg) as a brown solid. The product was used directly in the next reaction without purification.

MS m/z (ESI): 562.3 [M+1].

### Step 5

34d (240 mg, 427 μmol) was dissolved in 5 mL of aqueous hydrochloric acid (6 M) and stirred at 40° C. for 10 hours. The reaction solution was added with 5 mL of water, and extracted with dichloromethane (10 mL×3). The aqueous phase was lyophilized to obtain the hydrochloride of the title product 34e (200 mg) as a yellow solid. The product was used directly in the next step without purification.

MS m/z (ESI): 462.3 [M+1].

### Step 6

34e hydrochloride (20.0 mg, 40.1 μmol) and glycolic acid (4.6 mg, 60.2 μmol) were dissolved in 2 mL of N,N-dimethylformamide, added with N,N-diisopropylethylamine (15.5 mg, 120 μmol), 1-(3-dimethylaminopropyl)-3-ethylcarbodiimide hydrochloride (11.5 mg, 60.2 μmol) and 1-hydroxybenzotriazole (8.1 mg, 60.2 μmol), and stirred at 25° C. for 2 hours. The reaction solution was added with 1 mL of water, and extracted with dichloromethane (3 mL×3). The organic phase was dried over anhydrous sodium sulfate and filtered, and the filtrate was concentrated by vacuum distillation. The residue was purified by pre-HPLC (separation conditions: chromatographic column: Phenomenex Luna C18 150×25 mm×10 μm, mobile phase: A-water (0.225% formic acid), B-acetonitrile, gradient elution, B %: 26%-56%) to obtain the title product 34 (2.51 mg, yield: 12%) as a yellow solid.

MS m/z (ESI): 520.3 [M+1]. ¹H NMR (400 MHz, CD₃OD) δ 7.78-7.62 (m, 2H), 5.64-5.55 (m, 1H), 5.47-5.32 (m, 3H), 5.20-5.10 (m, 2H), 4.15-4.04 (m, 2H), 3.74-3.63 (m, 2H), 3.21-3.10 (m, 2H), 2.40-2.29 (m, 2H), 1.96-1.82 (m, 2H), 0.94-0.86 (m, 1H), 0.51-0.38 (m, 2H), 0.15-−0.02 (m, 2H).

### Example 2-35

### (S)-N-((4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxyacetamide 35

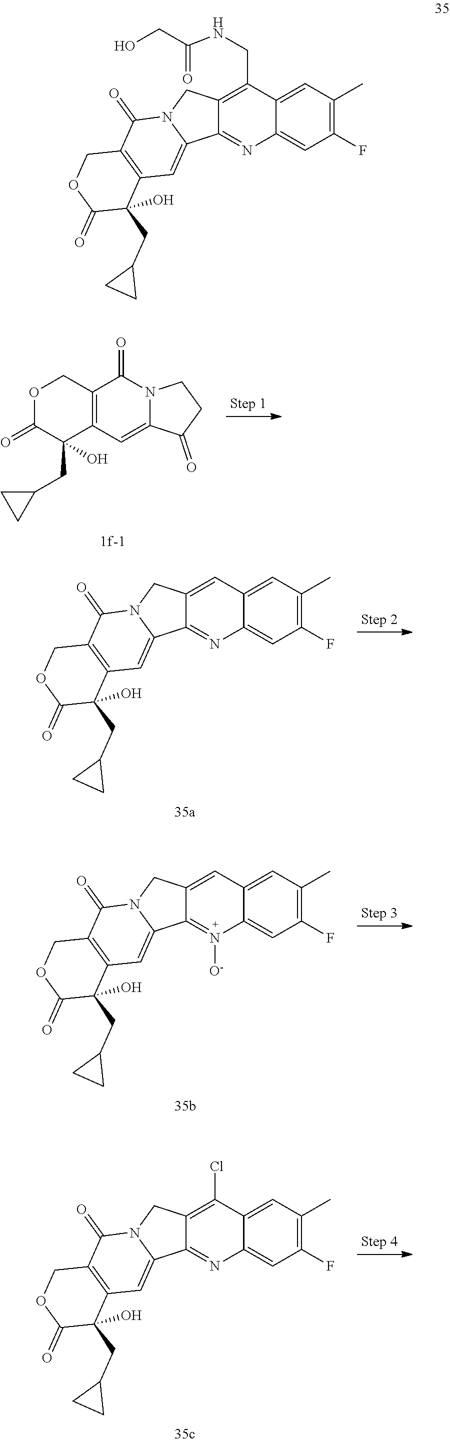

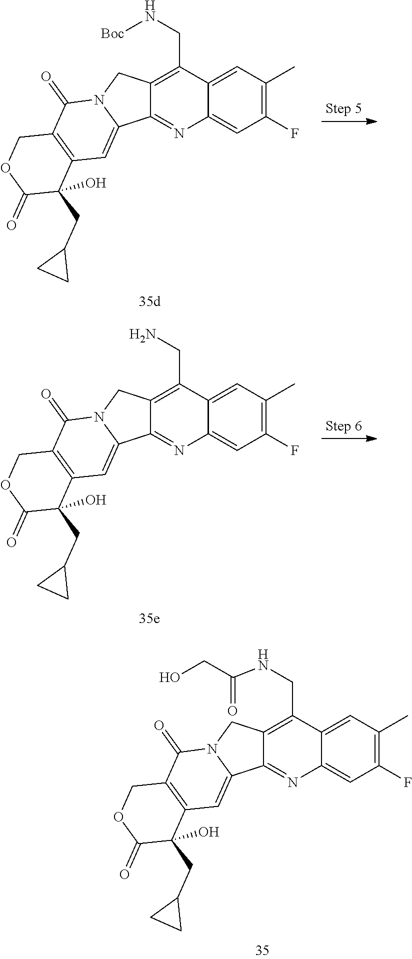 [SMILES: Cc1cc2c(CNC(=O)OC(C)(C)C)c3c(nc2cc1F)-c1cc2c(c(=O)n1C3)COC(=O)[C@]2(O)CC1CC1 ; Cc1cc2c(CN)c3c(nc2cc1F)-c1cc2c(c(=O)n1C3)COC(=O)[C@]2(O)CC1CC1 ; Cc1cc2c(CNC(=O)CO)c3c(nc2cc1F)-c1cc2c(c(=O)n1C3)COC(=O)[C@]2(O)CC1CC1]

### Step 1

2-Amino-4-fluoro-5-methylbenzaldehyde hydrochloride (138 mg, 686 μmol) and 1f-1 (200 mg, 686 μmol) were dissolved in 35 mL of toluene, added with pyridium 4-toluenesulfonate (103 mg, 412 μmol) and o-methylphenol (596 mg, 5.52 mmol), and stirred at 135° C. for 3 hours. The reaction solution was concentrated by vacuum distillation, and the residue was purified by silica gel column chromatography using the eluent system A to obtain the title product 35a (213 mg, yield: 73%) as a yellow solid.

MS m/z (ESI): 407.2 [M+1].

### Step 2

35a (250 mg, 479 μmol) was dissolved in 12 mL of acetic acid, added with hydrogen peroxide (4.41 g, 38.9 mmol, the content is 30%), and stirred at 70° C. for 1.5 hours. At 0° C., the reaction solution was quenched with 20 mL of sodium thiosulfate, extracted with ethyl acetate (25 mL×2), the organic phase was washed with saturated sodium chloride solution (25 mL×2), the organic phase was dried over anhydrous sodium sulfate and filtered, and the filtrate was concentrated by vacuum distillation. The residue was purified by silica gel column chromatography using the eluent system A to obtain the title product 35b (120 mg, yield: 55%) as a yellow solid.

MS m/z (ESI): 423.2 [M+1].

### Step 3

35b (120 mg, 263 μmol) was dissolved in 2 mL of N,N-dimethylformamide, cooled to 0° C. in ice-water bath under nitrogen protection, added with oxalyl chloride (167 mg, 1.32 mmol), and stirred at 0° C. for 1 hour. The reaction solution was poured into 10 mL of water to precipitate solid, and the solid was collected by filtration, and dried to obtain the title product 35c (115 mg) as a yellow solid. The product was used directly in the next step without purification.

MS m/z (ESI): 441.1 [M+1].

### Step 4

35c (90.0 mg, 167 μmol) was dissolved in a mixed solvent of 3 mL of dioxane and 0.2 mL of water, added with potassium (((tert-butoxycarbonyl)amino)methyl)trifluoroborate (198 mg, 838 μmol), 1,1-bis(diphenylphosphino)ferrocenepalladium dichloride (24.5 mg, 33.5 μmol) and cesium carbonate (163 mg, 503 μmol), and stirred at 105° C. for 5 hours under nitrogen protection. The reaction solution was added with 5 mL of water, and extracted with ethyl acetate (8 mL×3). The organic phase was dried over anhydrous sodium sulfate and filtered, and the filtrate was concentrated by vacuum distillation to obtain the title product 35d (110 mg) as a yellow solid. The product was used directly in the next step without purification.

MS m/z (ESI): 536.1 [M+1].

### Step 5

35d (110 mg, 205 μmol) was dissolved in 3 mL of aqueous hydrochloric acid solution (6 M), and stirred at 40° C. for 10 hours. The reaction solution was added with 5 mL of water and and extracted with dichloromethane (10 mL×3), the aqueous phase was lyophilized to obtain the hydrochloride salt of the title product 35e (70 mg) as a yellow solid. The product was used directly in the next reaction without purification.

MS m/z (ESI): 436.0 [M+1].

### Step 6

The hydrochloride of 35e (30.0 mg, 68.8 μmol) and glycolic acid (5.76 mg, 75.7 μmol) were dissolved in 2 mL of N,N-dimethylformamide, added with N,N-diisopropylethylamine (26.7 mg, 206 μmol), 1-(3-dimethylaminopropyl)-3-ethylcarbodiimide hydrochloride (19.8 mg, 103 μmol) and 1-hydroxybenzotriazole (13.9 mg, 103 μmol), and stirred at 25° C. for 1.5 hours. The reaction solution was added with 2 mL of water, and extracted with dichloromethane (5 mL×3). The organic phase was dried over anhydrous sodium sulfate and filtered, and the filtrate was concentrated by vacuum distillation. The residue was purified by pre-HPLC (separation conditions: chromatographic column: Phenomenex Luna C18 150×25 mm×10 μm, mobile phase: A-water (0.225% formic acid), B-acetonitrile, gradient elution, B %: 19%-49%) to obtain the title product 35 (1.63 mg, yield: 5%) as a yellow solid.

MS m/z (ESI): 494.3 [M+1].

¹H NMR (400 MHz, CD₃OD) δ 8.34-8.29 (m, 1H), 7.83-7.77 (m, 1H), 7.73-7.70 (m, 1H), 5.64-5.58 (m, 1H), 5.56-5.49 (m, 2H), 5.44-5.37 (m, 1H), 5.07-5.01 (m, 2H), 4.06-3.99 (m, 2H), 2.62-2.51 (m, 3H), 1.95-1.83 (m, 2H), 0.93-0.88 (m, 1H), 0.50-0.38 (m, 2H), 0.15-0.08 (m, 1H), 0.07-−0.01 (m, 1H).

### Example 2-36

### (S)-N-(((S)-4-(Cyclopropylmethyl)-8-fluoro-4-hydroxy-9-methyl-3,14-dioxo-3,4,12,14-tetra hydro-1H-pyrano[3′,4′:6,7]indolizino[1,2-b]quinolin-11-yl)methyl)-2-hydroxypropanamide 36

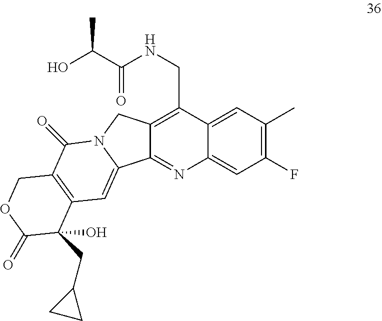 [SMILES: Cc1cc2c(CNC(=O)[C@H](C)O)c3c(nc2cc1F)-c1cc2c(c(=O)n1C3)COC(=O)[C@]2(O)CC1CC1]

The title compound 36 was prepared through 1f-1 and 2-amino-4-fluoro-5-methylbenzaldehyde (prepared by applying the method disclosed in Example 1 on page 73 of patent application “WO2022140504 A1”) with reference to the methods of steps 6 to 11 of Example 2-24.

Specifically, (2S)-2-hydroxypropionic acid (2.1 mg, 23.3 μmol) was dissolved in 0.5 mL of N,N-dimethylformamide, then added 1H-benzotriazol-1-yloxytripyrrolidinophosphonium hexafluorophosphate (13.1 mg, 25.2 μmol), N,N-diisopropylethylamine (14.9 mg, 155 μmol) and 35e (10.2 mg, 23.4 μmol) in sequence, and stirred at 25° C. for 0.5 h. The reaction solution was concentrated by vacuum distillation, and the obtained residue was purified by pre-HPLC (separation conditions: chromatographic column: InfinityLab Poroshell 120 SB-C18 21.2×150 mm, 4 um, mobile phase: A-water (0.1% formic acid), B-acetonitrile, gradient elution, B %: 10%-70%) to obtain the title product 36 (5.3 mg, yield: 44.56%) as a white solid.

MS m/z (ESI): 508.4 [M+1].

¹H NMR (400 MHz, DMSO-d6) δ 8.71 (t, J=6.0 Hz, 1H), 8.42 (d, J=8.3 Hz, 1H), 7.89 (d, J=10.8 Hz, 1H), 7.38 (s, 1H), 6.58 (s, 1H), 5.61 (d, J=4.9 Hz, 1H), 5.49 (d, J=2.1 Hz, 2H), 5.43 (d, J=2.6 Hz, 2H), 4.92-4.75 (m, 2H), 4.05-3.94 (m, 1H), 3.09-2.95 (m, 4H), 1.89-1.75 (m, 2H), 1.75-1.68 (m, 4H), 1.20 (d, J=6.8 Hz, 3H), 0.86-0.76 (m, 1H), 0.41-0.25 (m, 2H), 0.11-0.02 (m, 1H), −0.04-−0.13 (m, 1H).

### Example 2-37

### (R)-N-(((S)-4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxypropanamide 37

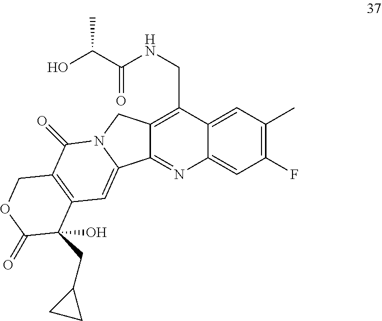 [SMILES: Cc1cc2c(CNC(=O)[C@@H](C)O)c3c(nc2cc1F)-c1cc2c(c(=O)n1C3)COC(=O)[C@]2(O)CC1CC1]

The title compound 37 was prepared through 35e and 2-amino-4-fluoro-5-methylbenzaldehyde (prepared by applying the method disclosed in Example 1 on page 73 of patent application “WO2022140504 A1”), with reference to the methods of steps 6 to 11 of Example 2-24.

Specifically, (2R)-2-hydroxypropionic acid (2.1 mg, 23.3 μmol) was dissolved in 0.5 mL of N,N-dimethylformamide, then added with 1H-benzotriazol-1-yloxytripyrrolidinophosphonium hexafluorophosphate (13.1 mg, 25.2 μmol), N,N-diisopropylethylamine (14.9 mg, 155 μmol) and 35e (10.2 mg, 23.4 μmol) in sequence, and stirred at 25° C. for 0.5 hours. The reaction solution was concentrated by vacuum distillation, and the residue was purified by pre-HPLC (separation conditions: chromatographic column: InfinityLab Poroshell 120 SB-C18 21.2×150 mm, 4 um, mobile phase: A-water (0.1% formic acid), B-acetonitrile, gradient elution, B %: 10%-70%) to obtain the title product 37 (3.6 mg, yield: 30.27%) as a white solid.

MS m/z (ESI): 508.4 [M+1].

¹H NMR (400 MHz, DMSO-d6) δ 8.71 (s, 1H), 8.42 (d, J=8.2 Hz, 1H), 7.89 (d, J=10.8 Hz, 1H), 7.38 (s, 1H), 6.59 (s, 1H), 5.61 (d, J=4.9 Hz, 1H), 5.49 (s, 2H), 5.43 (d, J=3.0 Hz, 2H), 4.91-4.75 (m, 2H), 4.04-3.96 (m, 1H), 3.03-3.00 (m, 4H), 1.89-1.77 (m, 2H), 1.76-1.73 (m, 4H), 1.19 (d, J=6.8 Hz, 3H), 0.85-0.76 (m, 1H), 0.40-0.25 (m, 2H), 0.12-0.03 (m, 1H), −0.04-−0.13 (m, 1H).

### Example 2-38

### (S)-2-Cyclopropyl-N-(((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxyacetamide 38

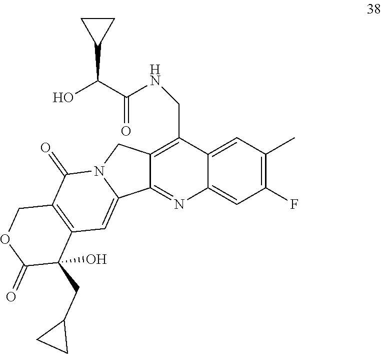 [SMILES: Cc1cc2c(CNC(=O)[C@@H](O)C3CC3)c3c(nc2cc1F)-c1cc2c(c(=O)n1C3)COC(=O)[C@]2(O)CC1CC1]

By using 35e and 2-amino-4-fluoro-5-methylbenzaldehyde (prepared by applying the method disclosed in Example 1 on page 73 of patent application “WO2022140504 A1”), the title compound 38 was prepared by referring to the methods of steps 6 to 11 of Example 2-24.

### Example 2-39

### (R)-2-Cyclopropyl-N-(((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxyacetamide 39

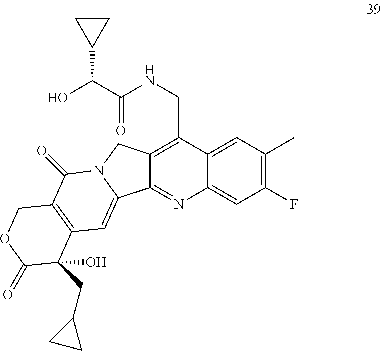 [SMILES: Cc1cc2c(CNC(=O)[C@H](O)C3CC3)c3c(nc2cc1F)-c1cc2c(c(=O)n1C3)COC(=O)[C@]2(O)CC1CC1]

By using 35e and 2-amino-4-fluoro-5-methylbenzaldehyde (prepared by applying the method disclosed in Example 1 on page 73 of patent application “WO2022140504 A1”), the title compound 39 was prepared by the same methods of steps 6 to 11 of Example 2-24.

### III. Preparation Examples of Conjugate Intermediate Linker-Drugs

### Example 3-1

### 2-(((S)-10-Benzyl-1-(((1 S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-1

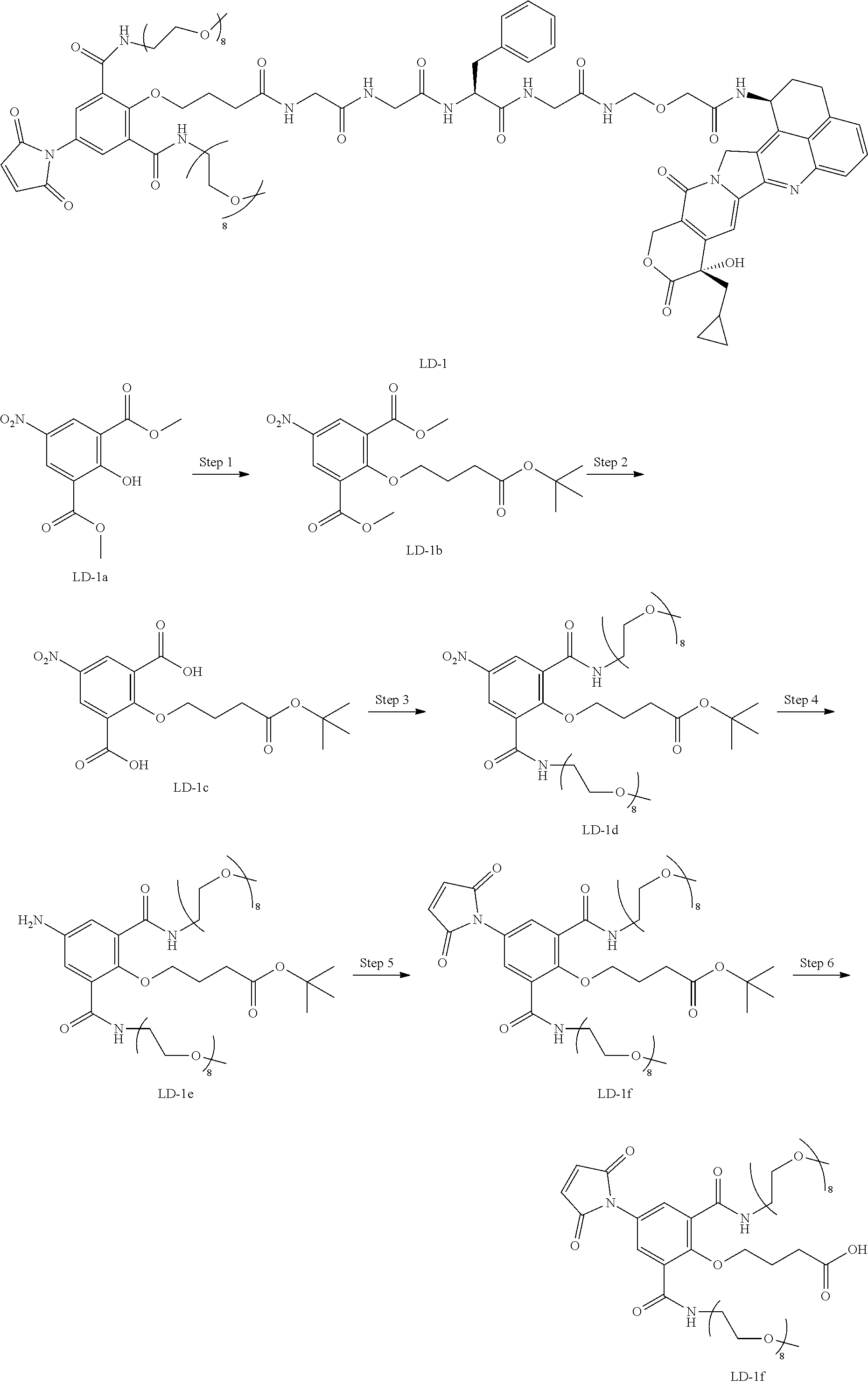

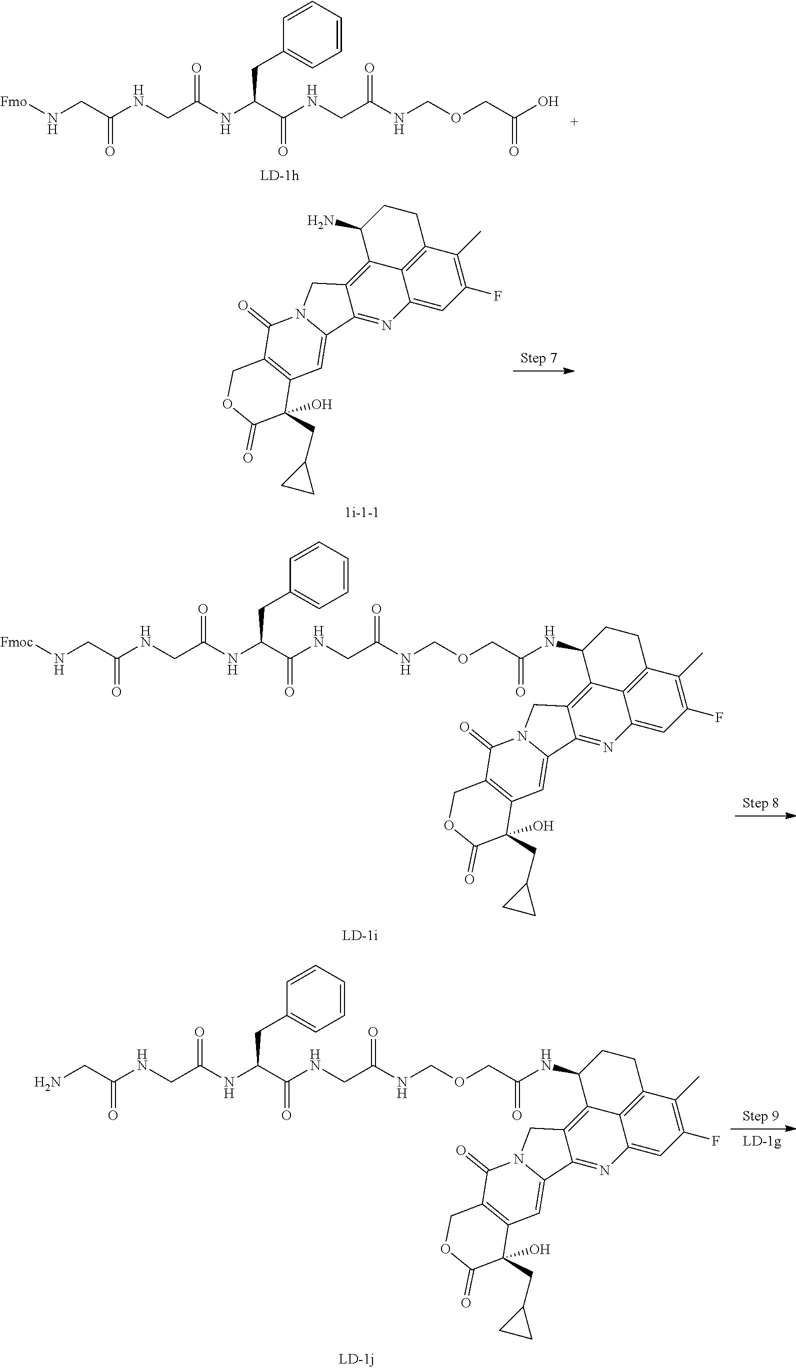

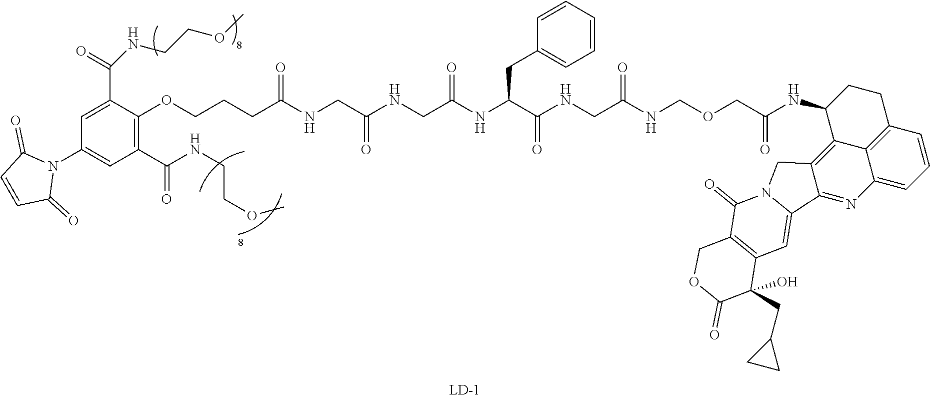

### Step 1

### Dimethyl 2-(4-(tert-Butyloxy)-4-oxobutyloxy)-5-nitroisophthalate LD-1b

LD-1a (5 g, 19.55 mmol, prepared by applying the method disclosed in “Journal of Physical Organic Chemistry, 2010, vol. 16, #10, p. 682-690”), tert-butyl 4-hydroxybutyrate (3.76 g, 23.47 mmol) and triphenylphosphine (7.69 g, 29.33 mmol) were dissolved in a mixed solvent of 40 mL of N,N-dimethylformamide and 120 mL of tetrahydrofuran, cooled to 0° C. under nitrogen protection in ice-water bath. The reaction solution was added with diisopropyl azodicarboxylate (5.93 g, 29.33 mmol) slowly and dropwise, and stirred at 25° C. for 20 hours. 150 mL of water was added, and the diluted reaction solution was extracted with ethyl acetate (200 mL×3). The organic phase was dried over anhydrous sodium sulfate and filtered, and the filtrate was concentrated by vacuum distillation. The residue was purified by reverse phase high performance liquid chromatography (separation conditions: chromatographic column: I.D.100 mm×H300 mm Welch Ultimate XB_C18 20-40 μm, mobile phase: A-water, B-acetonitrile, gradient elution, flow rate: 200 mL/min, instrument: Agela Astra) to obtain the title product LD-1b (4.8 g, yield: 61%) as a yellow solid.

MS m/z (ESI): 420.2 [M+23].

### Step 2

### 2-(4-(tert-Butoxy)-4-oxobutoxy)-5-nitroisophthalic acid LD-1c

LD-1b (1.5 g, 3.71 mmol) was dissolved in a mixed solvent of 15 mL of water and 15 mL of tetrahydrofuran, added with lithium hydroxide monohydrate (311.10 mg, 7.41 mmol), and stirred at 25° C. for 2 hours. 1N hydrochloric acid solution was added to the reaction solution to adjust the pH value to 2-3, and ethyl acetate was used for extraction (40 mL×3). The organic phase was dried over anhydrous sodium sulfate and filtered, and the filtrate was concentrated by vacuum distillation to obtain the crude title product LD-1c (1.49 g) as a yellow solid. The product was used directly in the next step without purification.

MS m/z (ESI): 392.1 [M+23].

### Step 3

### tert-Butyl 4-(2,6-bis((2,5,8,11,14,17,20,23-octaoxapentacosan-25-yl)carbamoyl)-4-nitrophenoxy)butanoate LD-1d

LD-1c (400 mg, 933.60 μmol) was dissolved in 5 mL of N,N-dimethylformamide, added with 1-hydroxybenzotriazole (378.45 mg, 2.80 mmol), 1-(3-dimethylaminopropyl)-3-ethylcarbodiimide hydrochloride (536.92 mg, 2.80 mmol), N,N-diisopropylethylamine (723.97 mg, 5.60 mmol) and 3,6,9,12,15,18,21,24-octaoxapentacosane-1-amine (895.03 mg, 2.33 mmol), and stirred at 25° C. for 2 hours. 10 mL of water was added, and dichloromethane (10 mL×3) was used for extraction. The organic phase was dried over anhydrous sodium sulfate and filtered, the filtrate was concentrated by vacuum distillation, and the obtained residue was purified by silica gel column chromatography with the developing system B to obtain the title product LD-1d (515 mg, yield: 50%) as a yellow oil.

MS m/z (ESI): 1117.7 [M+18].

### Step 4

### tert-Butyl 4-(2,6-bis((2,5,8,11,14,17,20,23-octaoxapentacosan-25-yl)carbamoyl)-4-aminophenoxy)butanoate LD-1e

LD-1d (515 mg, 466.74 μmol) was dissolved in a mixed solvent of 20 mL of ethanol and 10 mL of water, added with iron powder (130.32 mg, 2.33 mmol) and ammonium chloride (249.66 mg, 4.67 mmol), and stirred at 80° C. for 16 hours. The reaction solution was filtered with Celite, 10 mL of water was added to the filtrate, the diluted filtrate was extracted with dichloromethane (10 mL×3), the organic phase was dried over anhydrous sodium sulfate and filtered, and the filtrate was concentrated by vacuum distillation. The obtained residue was purified by silica gel column chromatography with the eluent system A to obtain the title product LD-1e (435 mg, yield: 87%) as a yellow oil.

MS m/z (ESI): 1070.8 [M+1].

### Step 5

### tert-Butyl 4-(2,6-bis((2,5,8,11,14,17,20,23-octaoxapentacosan-25-yl)carbamoyl)-4-(2,5-dioxo-2,5-dihydro-1H-pyrrol-1-yl)phenoxy)butanoate LD-1f

LD-1e (380 mg, 352.47 μmol) and 2,5-furandione (38.02 mg, 387.71 μmol) were dissolved in 6 mL of dioxane, and stirred at 25° C. for 1 hour. After the completion of the reaction of the starting material LD-1e monitored by TLC, the reaction solution was added with ammonium persulfate (160.87 mg, 704.93 μmol) and dimethyl sulfoxide (55.08 mg, 704.93 μmol), and stirred at 100° C. for 1 hour. The reaction solution was concentrated by vacuum distillation, and the residue was purified by preparative HPLC (separation conditions: chromatographic column: Welch Xtimate C18 150×25 mm×5 μm; mobile phase: A-water (0.1% trifluoroacetic acid), B-acetonitrile, gradient elution, B %: 25%-55%, flow rate 25 mL/min) to obtain the title product LD-1f (91 mg, yield: 21%) as a yellow oil.

MS m/z (ESI): 1167.8 [M+18].

### Step 6

### 4-(2,6-Bis((2,5,8,11,14,17,20,23-octaoxapentacosan-25-yl)carbamoyl)-4-(2,5-dioxo-2,5-dihydro-1H-pyrrol-1-yl)phenoxy)butanoic acid LD-1 g

LD-1f (2 mg, 1.74 μmol) was dissolved in 0.5 mL of dichloromethane, added with trifluoroacetic acid (0.1 mL), and stirred at 25° C. for 1 hour. The reaction solution was concentrated by vacuum distillation to obtain the crude title product LD-1 g (1.5 mg) as a colorless oil. The product was used directly in the next step without purification.

MS m/z (ESI): 1111.8 [M+18].

### Step 7

### (9H-Fluoren-9-yl)methyl((S)-10-benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl) carbamate LD-1i

LD-1h (68.7 mg, 94.7 μmol, prepared by applying the method disclosed in Example LND1067-L1 on page 9 of patent application “CN113402584 A”) and 1i-1-1 (50.0 mg, 78.9 μmol, trifluoroacetate) were dissolved in 2 mL of N,N-dimethylformamide, added with 1-hydroxybenzotriazole (16.0 mg, 118 μmol), 1-(3-dimethylaminopropyl)-3-ethylcarbodiimide hydrochloride (22.7 mg, 118 μmol), N,N-diisopropylethylamine (30.6 mg, 236 μmol), and stirred at 25° C. for 3 hours. 1 mL of water was added, dichloromethane (3 mL×3) was used for extraction, the organic phase was dried over anhydrous sodium sulfate and filtered, and the filtrate was concentrated by vacuum distillation. The obtained residue was purified by silica gel column chromatography with the eluent system A to obtain the title product LD-1i (60 mg, yield: 57%) as a yellow solid.

MS m/z (ESI): 1089.6 [M+1].

### Step 8

### (S)-2-(2-(2-Aminoacetamido)acetamido)-N-(2-(((2-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)-3-phenylpropanamide LD-1j

LD-1i (55.0 mg, 41.5 μmol) was dissolved in 1.5 mL of acetonitrile, added with diethylamine (213 mg, 2.91 mmol), and stirred at 25° C. for 1 hour. The reaction solution was concentrated by vacuum distillation to obtain the crude title product LD-1j (35 mg) as a yellow solid. The product was used directly in the next step without purification.

MS m/z (ESI): 867.3 [M+1].

### Step 9

### 2-(((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazahenicosan-21-yl)oxy)-5-(2,5-dioxo -2,5-dihydro-1H-pyrrol-1-yl)-N¹,N³-bis(2,5,8,11,14,17,20,23-octaoxapentacosan-25-yl)-isophthalamide LD-1

The title compound LD-1 was obtained by using LD-1j and LD-1 g in the presence of 1-hydroxybenzotriazole, 1-(3-dimethylaminopropyl)-3-ethylcarbodiimide hydrochloride and N,N-diisopropylethylamine.

MS m/z (ESI): 972.5 [1/2(M+2)].

By referring to the method of Example 3-1, corresponding substrates were used to prepare LD-2 to LD-12:

### Example 3-2: Preparation of LD-2

### 2-(((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-2

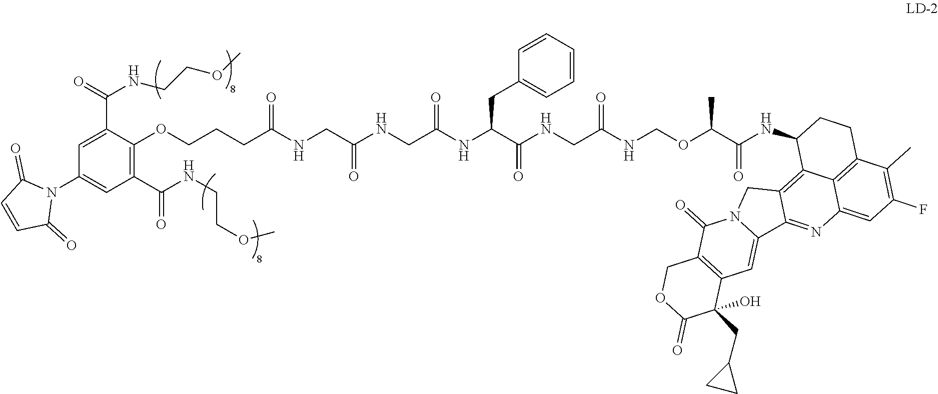

MS m/z (ESI): 979.5 [1/2(M+2)].

### Example 3-3: Preparation of LD-3

### 2-(((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinol in-1-yl)amino)-2-methyl-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-3

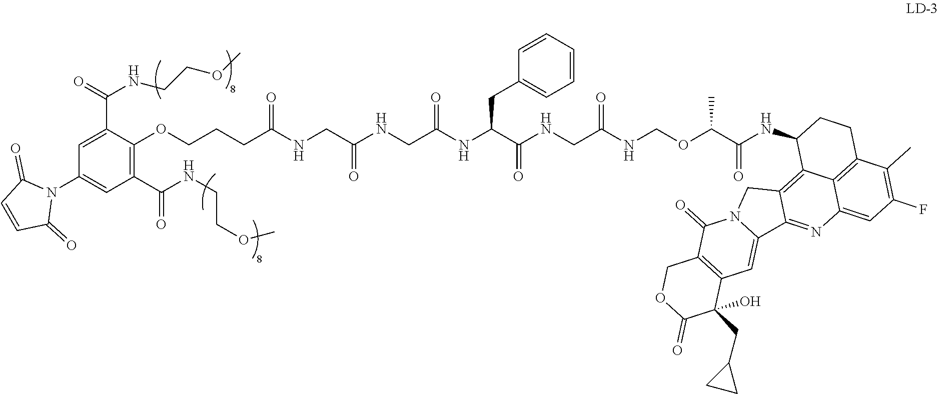

MS m/z (ESI): 979.6 [1/2(M+2)].

### Example 3-4: Preparation of LD-4

### 2-(((2S,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizin o[1,2-b]quinolin-1-yl)amino)-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-4

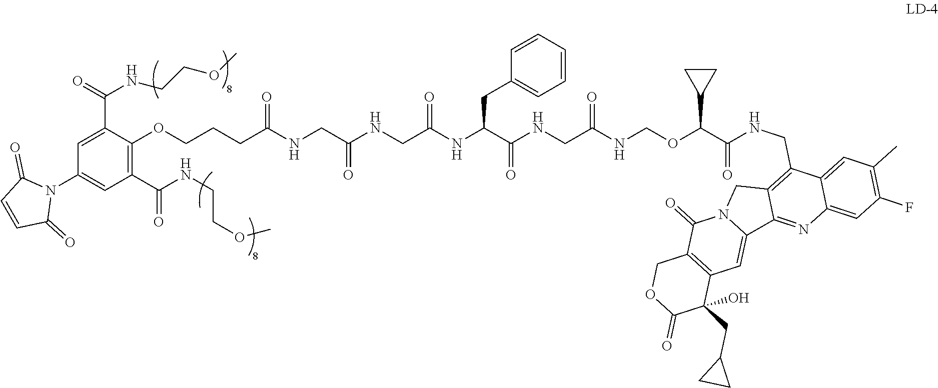

MS m/z (ESI): 992.5 [1/2(M+2)].

### Example 3-5: Preparation of LD-5

### 2-(((2R,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizin o[1,2-b]quinolin-1-yl)amino)-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-5

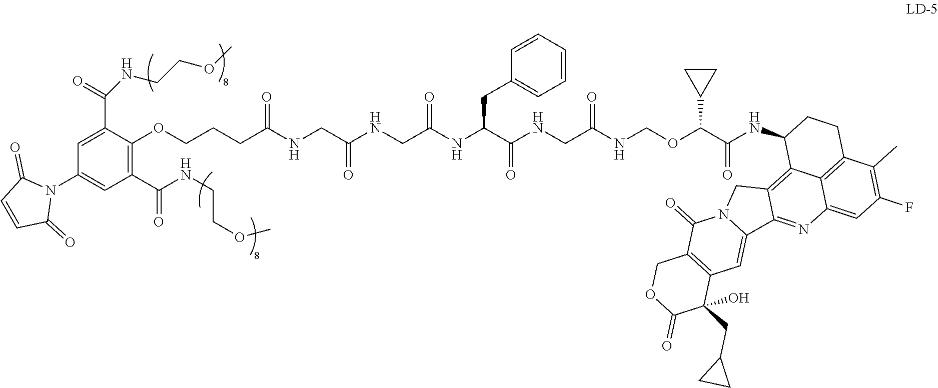

MS m/z (ESI): 992.5 [1/2(M+2)].

### Example 3-6: Preparation of LD-6

### 2-(((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazahenicosan-21-yl)oxy)-5-(2,5-diox o-2,5-dihydro-1H-pyrrol-1-yl)-3-fluoro-N-(2,5,8,11,14,17,20,23-octaoxapentacosan-25-yl)benzamide LD-6

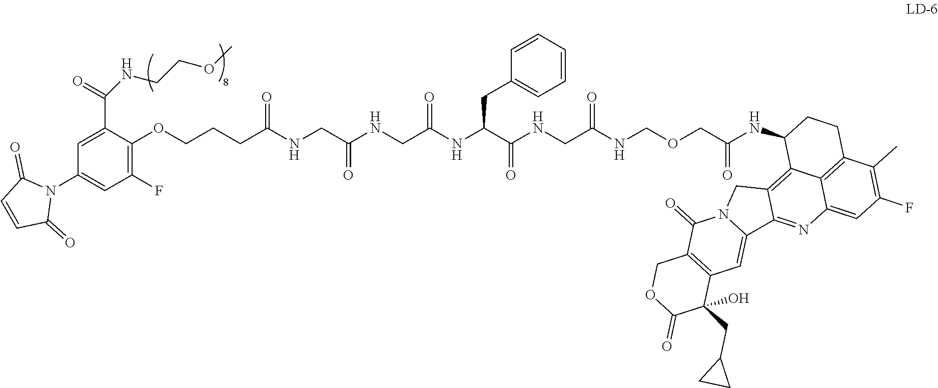

MS m/z (ESI): 776.8 [1/2(M+2)].

### Example 3-7: Preparation of LD-7

### 2-(((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15,18-hexaoxo-3-oxa-5,8,11,14,17-pentaazahenicosan-21-yl)o xy)-5-(2,5-dioxo-2,5-dihydro-1H-pyrrol-1-yl)-3-fluoro-N-(2,5,8,11,14,17,20,23-octaoxapentacosan-25-yl)benzamide LD-7

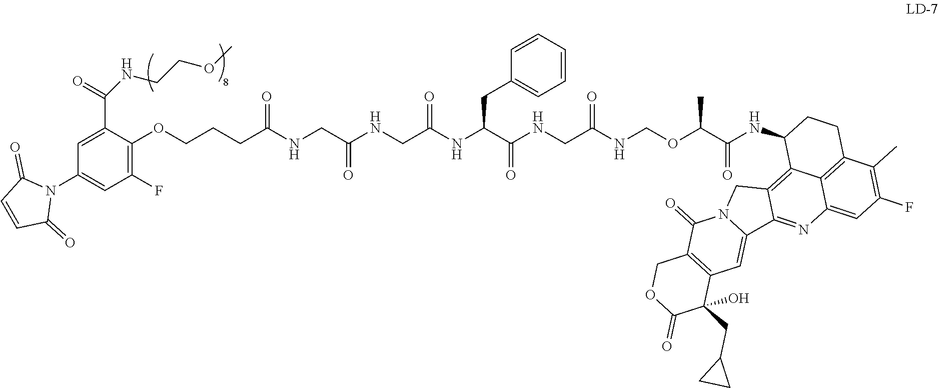

### Example 3-8: Preparation of LD-8

### 2-(((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinol in-1-yl)amino)-2-methyl-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-8

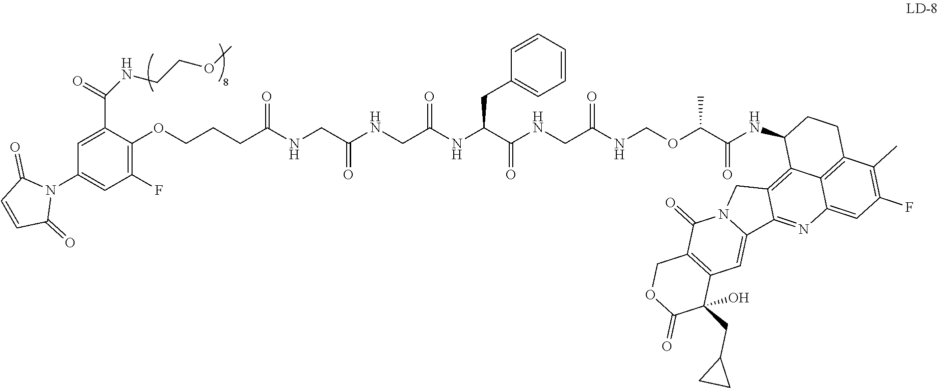

### Example 3-9: Preparation of LD-9

### 2-(((2S,10S)-10-benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-9

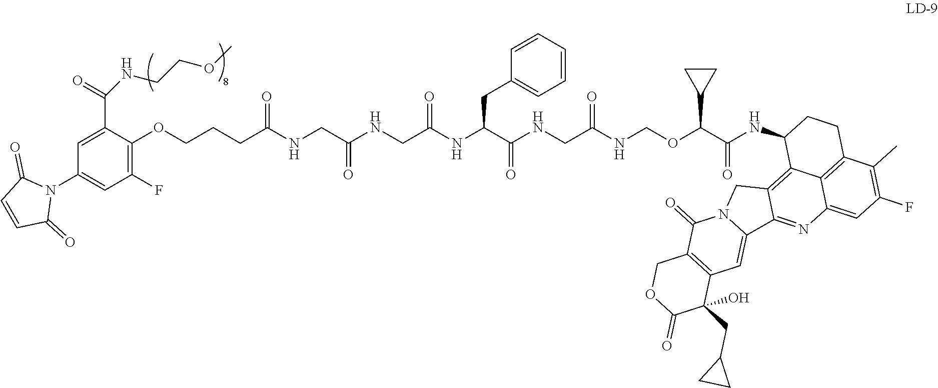

### Example 3-10: Preparation of LD-10

### 2-(((2R,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizin o[1,2-b]quinolin-1-yl)amino)-1,6,9,12,15,18-hexaoxo-3-oxa-5,8,11,14,17-pentaazahenicosan-21-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-10

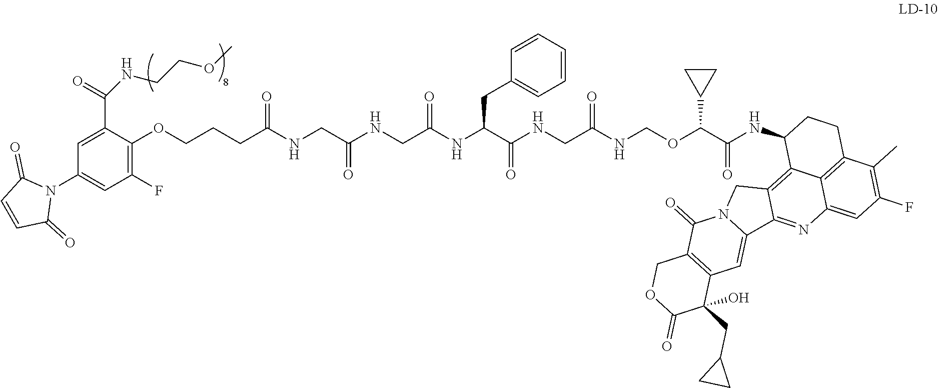

### Example 3-11: Preparation of LD-11

### N-((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl)hex-5-ynamide LD-11

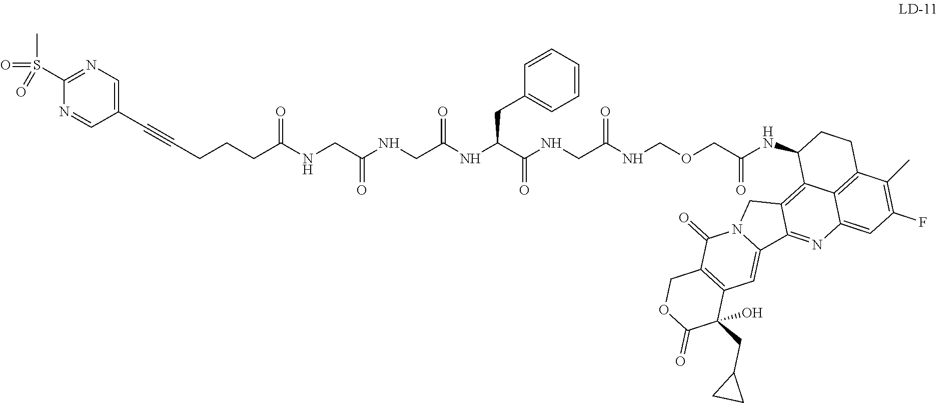 [SMILES: Cc1c(F)cc2nc3c(c4c2c1CC[C@@H]4NC(=O)COCNC(=O)CNC(=O)[C@H](Cc1ccccc1)NC(=O)CNC(=O)CNC(=O)CCCC#Cc1cnc(S(C)(=O)=O)nc1)Cn1c-3cc2c(c1=O)COC(=O)[C@]2(O)CC1CC1]

MS m/z (ESI): 559.8 [1/2(M+2)].

### Example 3-12: Preparation of LD-12

### (S)-N5-((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2-(methylsul fonyl)pyrimidin-5-yl)hex-5-ynamido)-N¹-(2,5,8,11,14,17,20,23-octaoxapentacosan-25-yl)pentanediamide LD-12

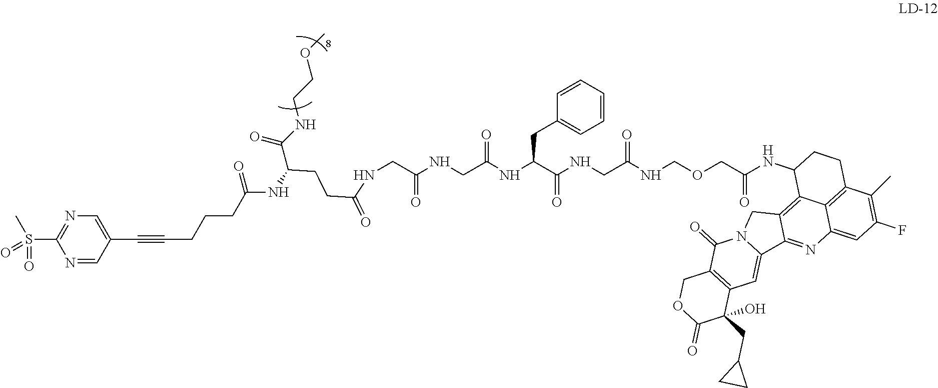

MS m/z (ESI): 806.9 [1/2(M+2)].

### Example 3-13: Preparation of LD-13

### (S)-N5-((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-13

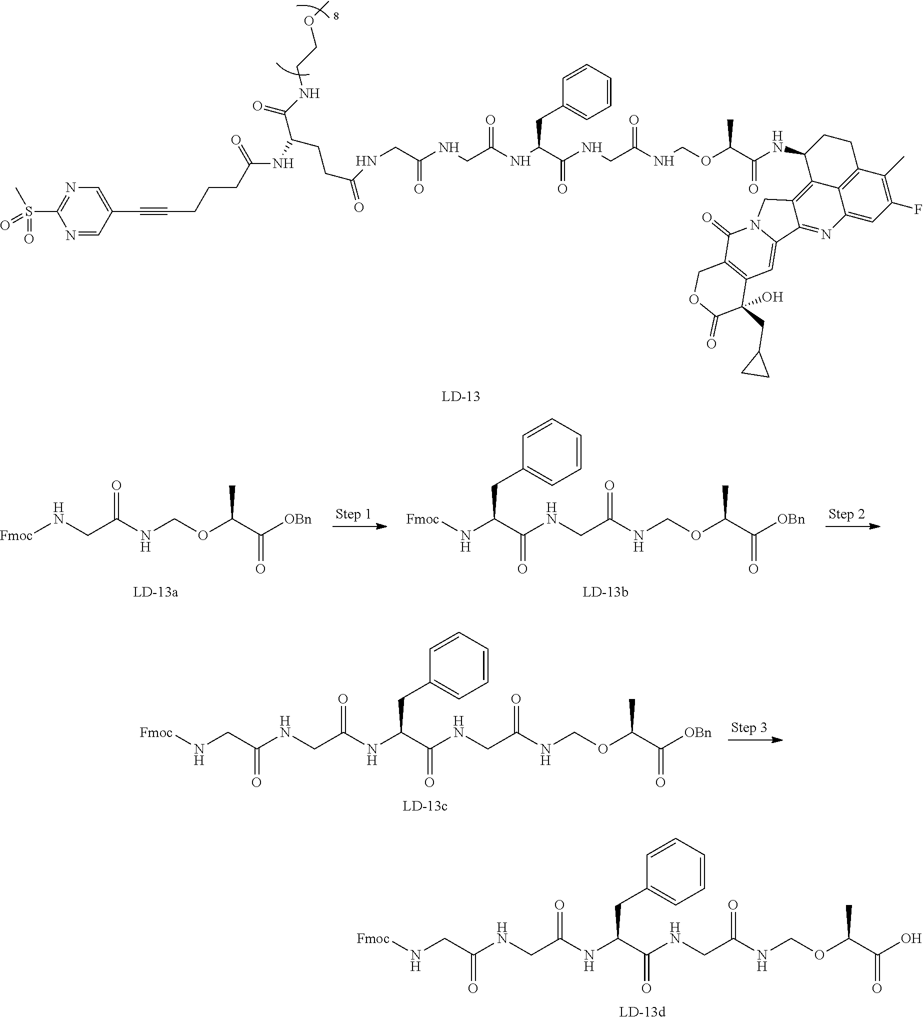

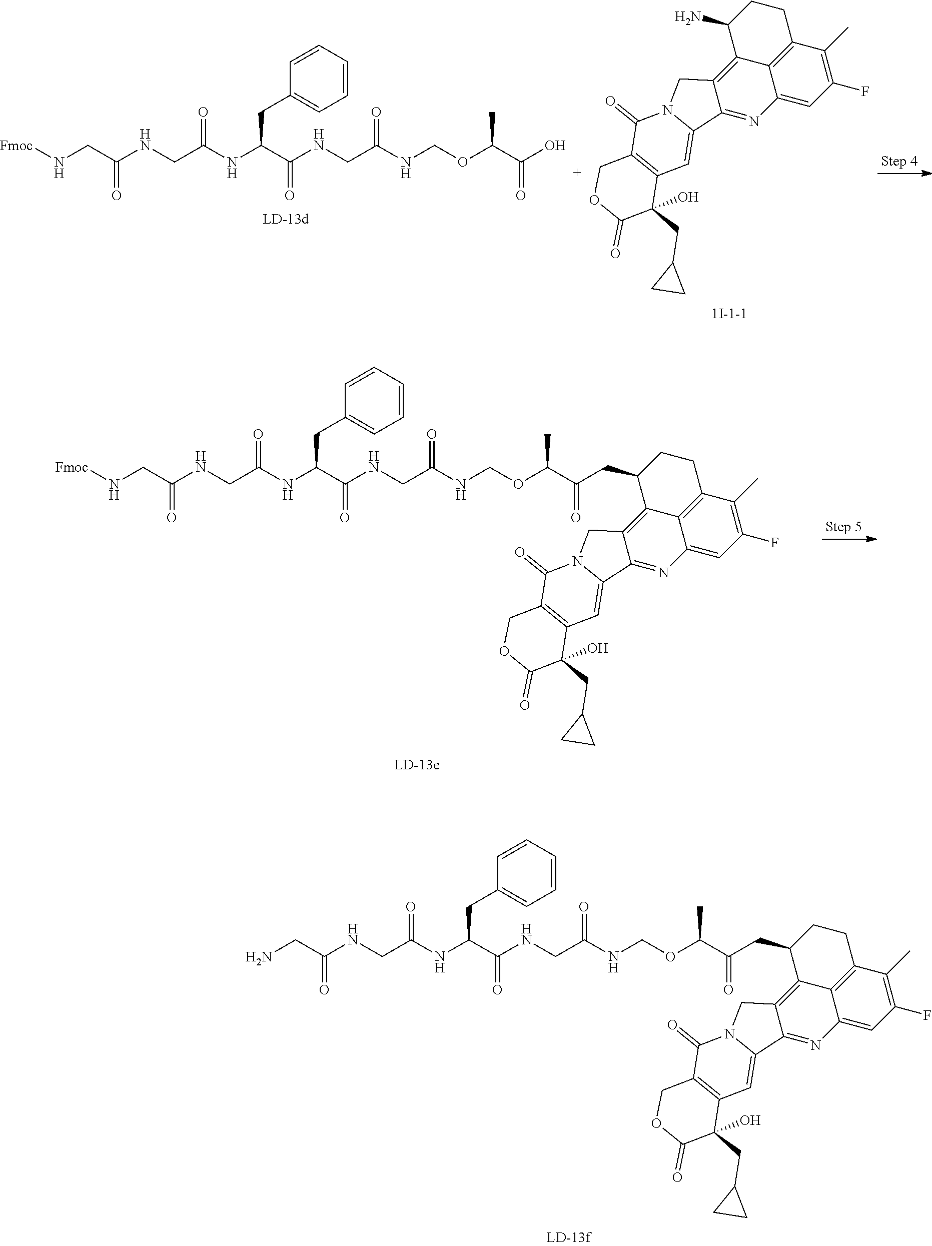

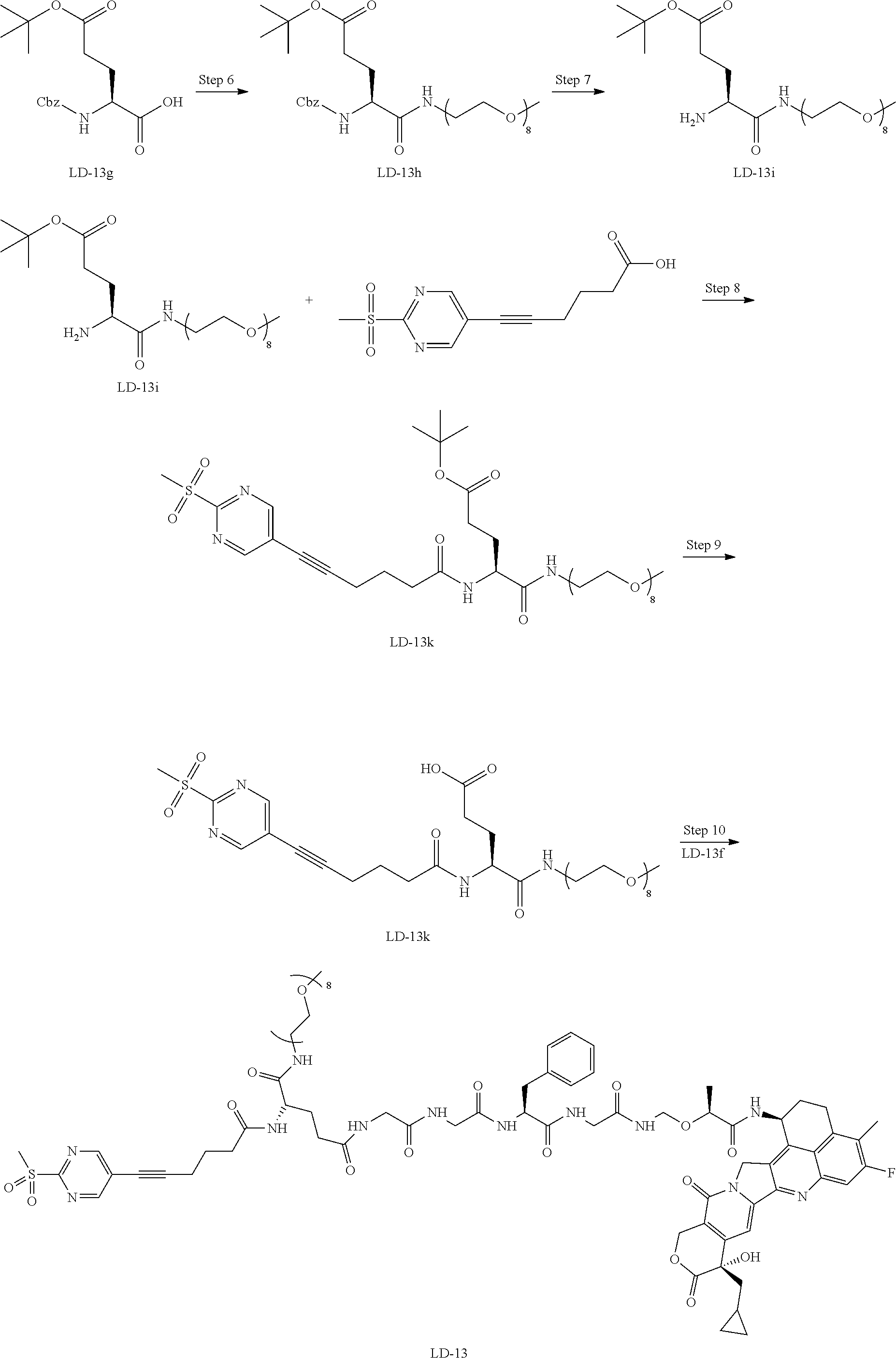

### Step 1

### Benzyl(5s,13S)-5-Benzyl-1-(9H-fluro-9-yl)-13-methyl-3,6,9-trioxo-2,12-dioxa-4,7,10-triazatetradecan-14-oate LD-13b

LD-13a (1.70 g, 3.17 mmol, prepared by applying the method disclosed in Example 3 on page 8 of patent application “WO2022056696 A1”) was dissolved in 20 mL of N,N-dimethylformamide, added with 1,8-diazabicyclo[5.4.0]undec-7-ene (241 mg, 1.59 mmol), and stirred at 20° C. for 1 hour. Then, (((9H-fluoren-9-yl)methoxy)carbonyl)-L-phenylalanine (1.16 g, 3.00 mmol), 1-hydroxybenzotriazole (608 mg, 4.51 mmol) and 1-(3-dimethylaminopropyl)-3-ethylcarbodiimide hydrochloride (863 mg, 4.51 mmol) were added to the reaction solution in sequence, and stirred at 25° C. for 1 hour. 50 mL of water was added, ethyl acetate (45 mL×3) was used for extraction, the organic phase was dried over anhydrous sodium sulfate and filtered, the filtrate was concentrated by vacuum distillation. The residue was purified by silica gel column chromatography using the eluent system A to obtain the title product LD-13b (1.35 g, yield: 65%) as a white solid.

MSm/z (ESI): 658.1 [M+23].

### Step 2

### Benzyl(11S,19S)-11-Benzyl-1-(9H-fluoren-9-yl)-19-methyl-3,6,9,12,15-pentaoxo-2,18-dioxa-4,7,10,13,16-pentaazaicosan-20-oate LD-13c

LD-13b (1.25 g, 1.81 mmol) was dissolved in 20 mL of N,N-dimethylformamide, added with 1,8-diazabicyclo[5.4.0]undec-7-ene (165 mg, 1.09 mmol), and stirred at 25° C. for 1 hour. Then, (((9H-fluoren-9-yl)methoxy)carbonyl)glycylglycine (750 mg, 2.12 mmol) and O-(7-azabenzotriazol-1-yl)-N,N,N′,N′-tetramethyluronium hexafluorophosphate (1.01 g, 2.65 mmol) were added to the reaction solution in sequence, and stirred at 25° C. for 1 hour. 50 mL of water was added, ethyl acetate (45 mL×3) was used for extraction, the organic phase was dried over anhydrous sodium sulfate and filtered, the filtrate was concentrated by vacuum distillation. The residue was purified by silica gel column chromatography with the eluent system A to obtain the title product LD-13c (850 mg, yield: 53%) as a white solid.

MS m/z (ESI): 767.4 [M+18].

### Step 3

### (11S,19S)-11-Benzyl-1-(9H-fluoren-9-yl)-19-methyl-3,6,9,12,15-pentaoxo-2,18-dioxa-4,7,10,13,16-pentaazaicosan-20-oic acid LD-13d

Under nitrogen protection, LD-13c (750 mg, 817 μmol) and palladium-on-carbon (434 mg, 10%, wetted with ca. 55% water) were dissolved in 4 mL of ethyl acetate and 8 mL of ethanol, and subjected to replacement with hydrogen gas three times. The reaction solution was stirred at 25° C. under hydrogen (15 Psi) for 1 hour. The reaction solution was filtered with Celite, and the filtrate was concentrated by vacuum distillation to obtain the crude title product LD-13d (450 mg, yield: 67%) as a white solid. The product was used directly in the next reaction without purification.

MS m/z (ESI): 677.2 [M+18].

### Step 4

### (9H-Fluoren-9-yl)methyl((2S,10S)-10-benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)carbamate LD-13e

LD-13d (30.0 mg, 36.8 μmol) and 1i-1-1 trifluoroacetate (21.5 mg, 36.8 μmol) were dissolved in 1 mL of N,N-dimethylformamide, added with 1-hydroxybenzotriazole (7.47 mg, 55.2 μmol), 1-(3-dimethylaminopropyl)-3-ethylcarbodiimide hydrochloride (10.6 mg, 55.3 μmol) and N,N-diisopropylethylamine (14.2 mg, 110 μmol) in sequence, and stirred at 25° C. for 1 hour. 2 mL of water was added, dichloromethane (5 mL×3) was used for extraction, the organic phase was dried over anhydrous sodium sulfate and filtered, and the filtrate was concentrated by vacuum distillation. The obtained residue was purified by silica gel column chromatography with the eluent system A to obtain the title product LD-13e (32.8 mg, yield: 65%) as a yellow solid.

MS m/z (ESI): 1103.2 [M+1].

### Step 5

### (S)-2-(2-(2-Aminoacetamido)acetamido)-N-(2-(((((S)-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)oxy)methyl)amino)-2-oxoethyl)-3-phenylpropanamide LD-13f

LD-13e (32.0 mg, 23.5 μmol) was dissolved in 1 mL of acetonitrile, added with diethylamine (142 mg, 1.94 mmol), and stirred at 25° C. for 1 hour. The reaction solution was concentrated by vacuum distillation, and the residue was purified by silica gel column chromatography using the eluent system A to obtain the title product LD-13f (18 mg, yield: 87%) as a white solid.

MS m/z (ESI): 881.2 [M+1].

### Step 6

LD-13 g (1.0 g, 2.97 mmol) was dissolved in 10 mL of N,N-dimethylformamide, added with N,N-diisopropylethylamine (1.15 g, 8.90 mmol), 1-hydroxybenzotriazole (400 mg, 2.97 mmol) and 1-(3-dimethylaminopropyl)-3-ethylcarbodiimide hydrochloride (570 mg, 2.97 mmol) in sequence, and stirred at 0° C. for 15 minutes. The reaction solution was added with 3,6,9,12,15,18,21,24-octaoxapentacosan-1-amine (1.14 g, 2.97 mmol), gradually heated to 25° C. and stirred for 2 hours. 20 mL of water was added, and ethyl acetate (30 mL×3) was used for extraction. The organic phase was dried over anhydrous sodium sulfate and filtered, the filtrate was concentrated by vacuum distillation. The residue was purified by silica gel column chromatography using the eluent system A to obtain the title product LD-13h (1.28 g, yield: 62%) as a colorless oil.

MS m/z (ESI): 703.4 [M+1].

### Step 7

LD-13h (1.28 g, 1.82 mmol) was dissolved in 13 mL of methanol; under nitrogen protection, palladium-on-carbon (10%, wetted with ca. 55% water, 300 mg) was added and stirred at 20° C. for 1 hour. The reaction mixture was subjected to replacement with hydrogen three times, and stirred at 25° C. for 2 hours under hydrogen gas (15 Psi). The reaction mixture was filtered with Celite, and the filtrate was concentrated by vacuum distillation to obtain the crude title product LD-13i (900 mg, yield: 87%) as a colorless oil. The product was used directly in the next reaction without purification.

MS m/z (ESI): 569.3 [M+1].

### Step 8

LD-13i (300 mg, 0.53 mmol) was dissolved in 5 mL of N,N-dimethylformamide, added with 2-(7-azobenzotriazole)-N,N,N′,N′-tetramethyluronium hexafluorophosphate (200 mg, 0.53 mmol) and N,N-diisopropylethylamine (204 mg, 1.58 mmol), then added with LD-13j (142 mg, 0.53 mmol, prepared by applying the method disclosed in Example 2 on page 30 of patent application “WO2021228141 A1”), and stirred at 25° C. for 2 hours. 10 mL of water was added, ethyl acetate (15 mL×3) was used for extraction, the organic phase was dried over anhydrous sodium sulfate and filtered, the filtrate was concentrated by vacuum distillation, the residue was purified by silica gel column chromatography with the eluent system A to obtain the title product LD-13k (107.85 mg, yield: 25%) as a colorless oil.

MS m/z (ESI): 819.4 [M+1].

### Step 9

LD-13k (260 mg, 0.32 mmol) was dissolved in 5 mL of dichloromethane, added with 1 mL of trifluoroacetic acid, and stirred at 25° C. for 2 hours. The reaction solution was concentrated by vacuum distillation, and the residue was purified by pre-HPLC (separation conditions: chromatographic column: Sunfire Prep C18 OBD 10 μm, 19×250 mm, mobile phase: A-acetonitrile, B-water, gradient elution, A %: 35%-95%) to obtain the title product LD-131 (82.84 mg, yield: 34%) as a white solid.

MS m/z (ESI): 763.3 [M+1].

### Step 10

LD-131 (17.9 mg, 23.5 μmol) and LD-13f (18.0 mg, 20.4 μmol) were dissolved in 1 mL of N,N-dimethylformamide, added with 1-hydroxybenzotriazole (4.14 mg, 30.6 μmol), 1-(3-dimethylaminopropyl)-3-ethylcarbodiimide hydrochloride (5.88 mg, 30.6 μmol) and N,N-diisopropylethylamine (7.92 mg, 61.3 μmol) in sequence, and stirred at 20° C. for 1 hour. The reaction solution was concentrated by vacuum distillation, and the residue was purified by pre-HPLC (separation conditions: chromatographic column: Welch Xtimate C18 150×25 mm×5 μm, mobile phase: A-water (0.1% trifluoroacetic acid), B-acetonitrile, gradient elution, B %: 30%-60%) to obtain the title product LD-13 (2.09 mg, yield: 6%) as a purple solid.

MS m/z (ESI): 813.9 [1/2(M+2)].

MS m/z (ESI): 1625.5 [M+1].

¹H NMR (400 MHz, CD₃OD) δ 8.99-8.88 (m, 2H), 8.64-8.57 (m, 1H), 8.54-8.49 (m, 1H), 8.22-8.14 (m, 1H), 8.08-8.01 (m, 1H), 7.70-7.59 (m, 2H), 7.27-7.20 (m, 2H), 7.20-7.07 (m, 3H), 5.72-5.64 (m, 1H), 5.61-5.54 (m, 1H), 5.49-5.41 (m, 1H), 5.40-5.25 (m, 2H), 5.20-5.13 (m, 1H), 4.78-4.71 (m, 2H), 4.39-4.25 (m, 3H), 3.94-3.86 (m, 3H), 3.80-3.75 (m, 1H), 3.73-3.55 (m, 26H), 3.55-3.49 (m, 4H), 3.38-3.33 (m, 6H), 3.20-3.12 (m, 1H), 2.97-2.84 (m, 2H), 2.60-2.53 (m, 2H), 2.50-2.38 (m, 5H), 2.38-2.27 (m, 3H), 2.26-2.14 (m, 2H), 2.11-2.02 (m, 2H), 1.98-1.83 (m, 4H), 1.78-1.71 (m, 1H), 1.65-1.56 (m, 1H), 1.49-1.41 (m, 3H), 0.94-0.80 (m, 2H), 0.46-0.33 (m, 2H), 0.09-0.03 (m, 1H), 0.01-−0.07 (m, 1H).

By referring to the method of Example 3-13, corresponding substrates were used to prepare LD-14 to LD-16:

### Example 3-14: Preparation of LD-14

### (S)-N5-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-14

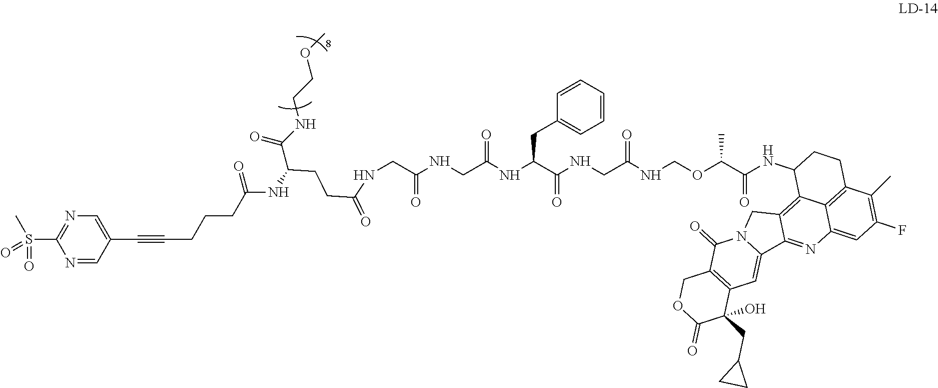

MS m/z (ESI): 813.9 [1/2(M+2)].

### Example 3-15: Preparation of LD-15

### (S)-N5-((2S,10S)-10-benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-15

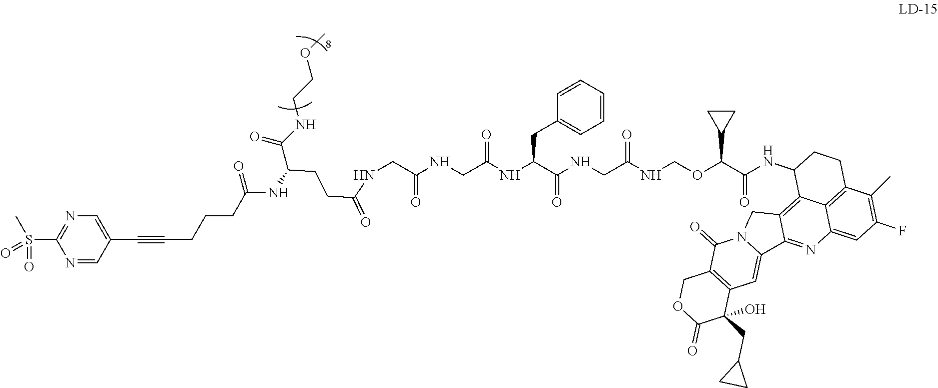

### Example 3-16: Preparation of LD-16

### (S)-N5-((2R,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-16

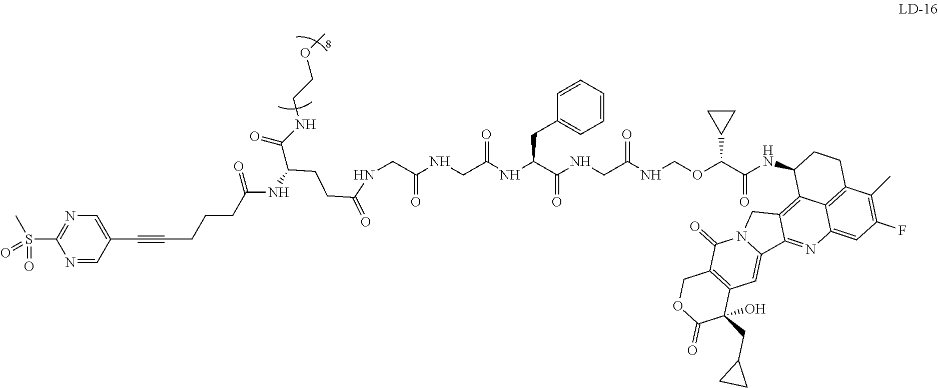

### Example 3-17: Preparation of LD-17

### (S)-N5-((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2, 5-dihydro-1H-pyrrol-1-yl)hexanamido)-N¹-(2,5,8,11,14,17,20,23-octaoxapentacosan-25-yl)pentanediamide LD-17

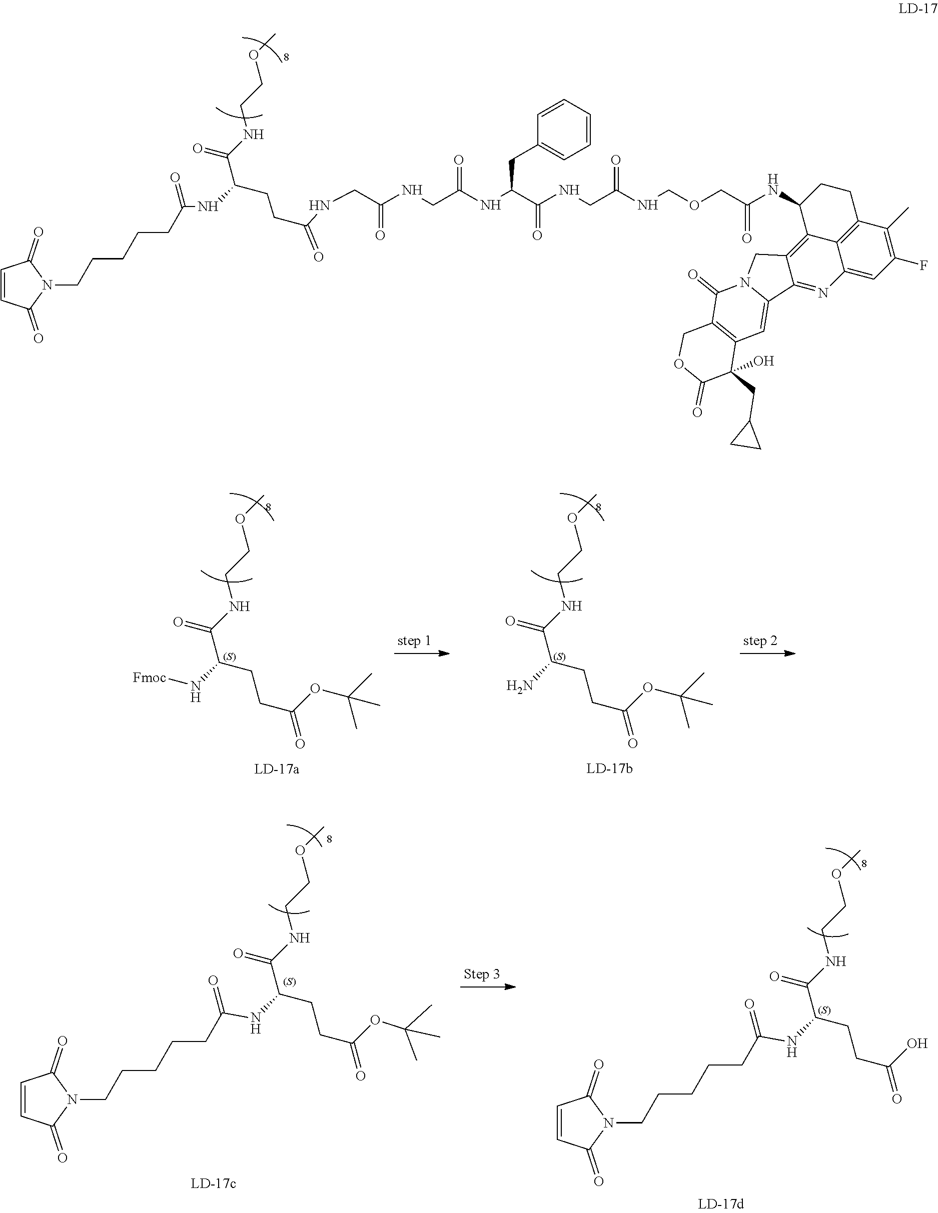

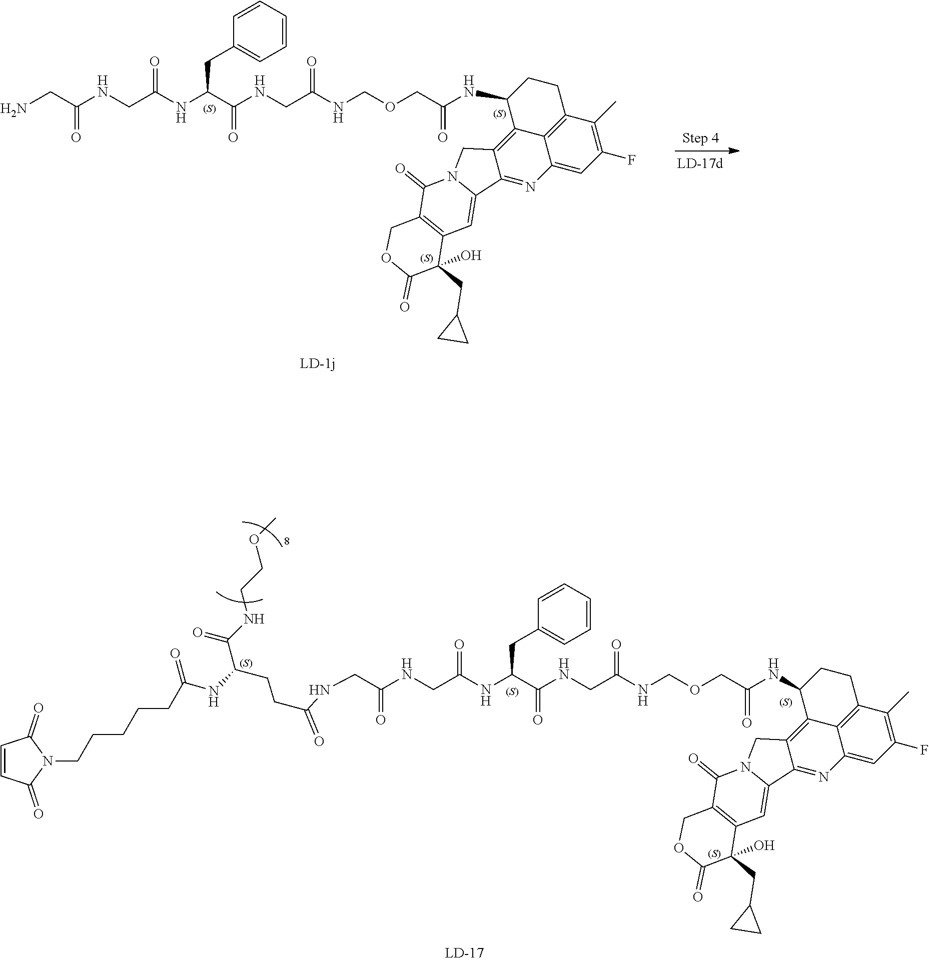

### Step 1

### tert-Butyl (S)-28-amino-27-oxo-2,5,8,11,14,17,20,23-octaoxa-26-azahentriacontan-31-oate LD-17b

LD-17a (75.0 mg, 92.0 μmol, prepared by applying the method disclosed in Example 2 on page 210 of patent application “WO2022262516 A1”) was dissolved in 1.5 mL of acetonitrile, added with 1,2-dichloroethane (375 mg, 3.80 mmol), and stirred at 25° C. for 1 hour. The reaction solution was concentrated by vacuum distillation to obtain the crude title product LD-17b (50 mg) as a yellow solid. The product was used directly in the next reaction without purification.

MS m/z (ESI): 569.4 [M+1].

### Step 2

### tert-Butyl (S)-28-(6-(2,5-dioxo-2,5-dihydro-1H-pyrrol-1-yl)hexanamido)-27-oxo-2,5,8,11,14,17,20,23-octaoxa-26-azahentriacontan-31-oate LD-17c

LD-17b (80.0 mg, 140 μmol) and 6-(2,5-dioxopyrrol-1-yl)hexanoic acid (44.5 mg, 211 μmol) were dissolved in 2 mL of dichloromethane, added with 1-hydroxybenzotriazole (28.5 mg, 211 μmol), 1-(3-dimethylaminopropyl)-3-ethylcarbodiimide hydrochloride (40.4 mg, 211 μmol) and N,N-diisopropylethylamine (54.5 mg, 422 μmol) in sequence, and stirred at 25° C. for 8 hours. 10 mL of water was added to the reaction solution, extracted with dichloromethane (15 mL×3), the organic phase was dried over anhydrous sodium sulfate and filtered, and the filtrate was concentrated by vacuum distillation. The residue was purified by pre-HPLC (separation conditions: chromatographic column: Welch Xtimate C18 150×25 mm×5 μm, mobile phase: A-water (0.1% trifluoroacetic acid), B-acetonitrile, gradient elution, B %: 23%-53%) to obtain the title product LD-17c (60 mg, yield: 90%) as a yellow jelly.

MS m/z (ESI): 762.4 [M+1].

### Step 3

### (S)-28-(6-(2,5-Dioxo-2,5-dihydro-1H-pyrrol-1-yl)hexanamido)-27-oxo-2,5,8,11,14,17,20,23-octaoxa-26-azahentriacontan-31-oic acid LD-17d

LD-17c (20.0 mg, 26.2 μmol) was dissolved in 1 mL of dichloromethane, added with trifluoroacetic acid (307 mg, 2.69 mmol), and stirred at 25° C. for 1 hour. The reaction solution was filtered, and the filtrate was concentrated by vacuum distillation. The residue was purified by pre-HPLC (separation conditions: chromatographic column: Welch Xtimate C18 150×25 mm×5 μm, mobile phase: A-water (0.1% trifluoroacetic acid), B-acetonitrile, gradient elution, B %: 10%-40%) to obtain the title product LD-17d (12 mg, yield: 64%) as a yellow colloid.

MS m/z (ESI): 706.4 [M+1].

### Step 4

LD-17d and LD-1j were used as starting materials to prepare the title product LD-17 by a method similar to that of LD-13.

By referring to the method of Example 3-13, corresponding substrates were used to prepare LD-18 to LD-96:

### Example 3-18: Preparation of LD-18

### (S)-N5-((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-(2,5,8,11,14,17,20,23-octaoxapent acosan-25-yl)pentanediamide LD-18

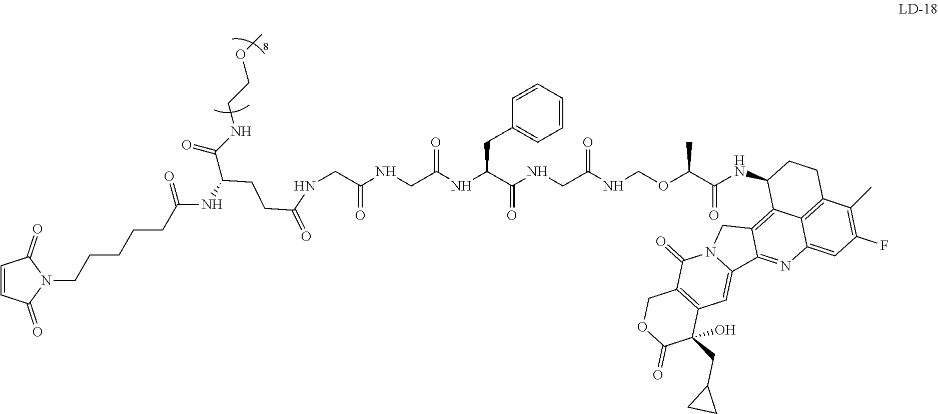

### Example 3-19: Preparation of LD-19

### (S)-N5-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-19

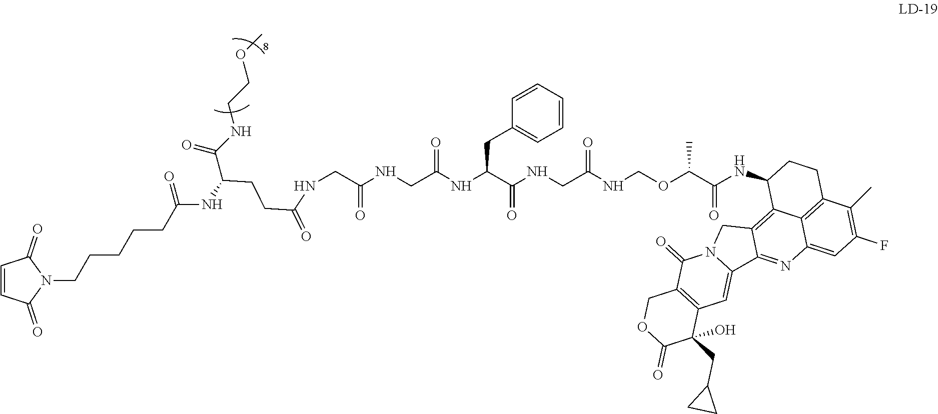

### Example 3-20 LD-20

### (S)-N5-((2S,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl) -2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-20

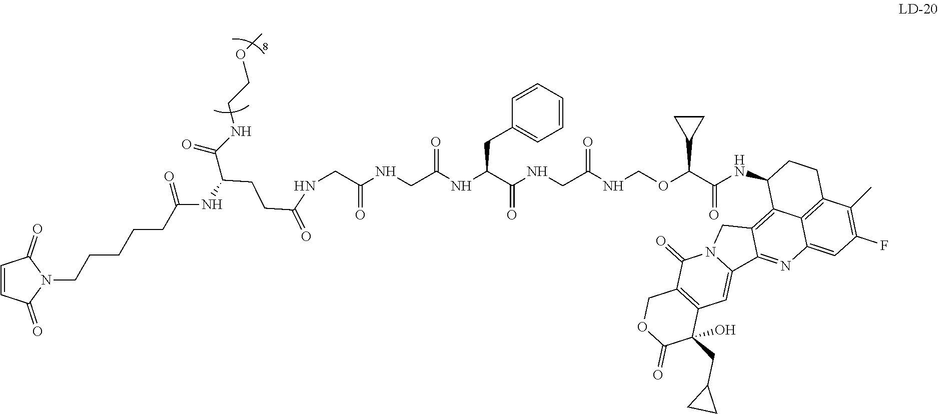

### Example 3-21: Preparation of LD-21

### (S)-N5-((2R,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-21

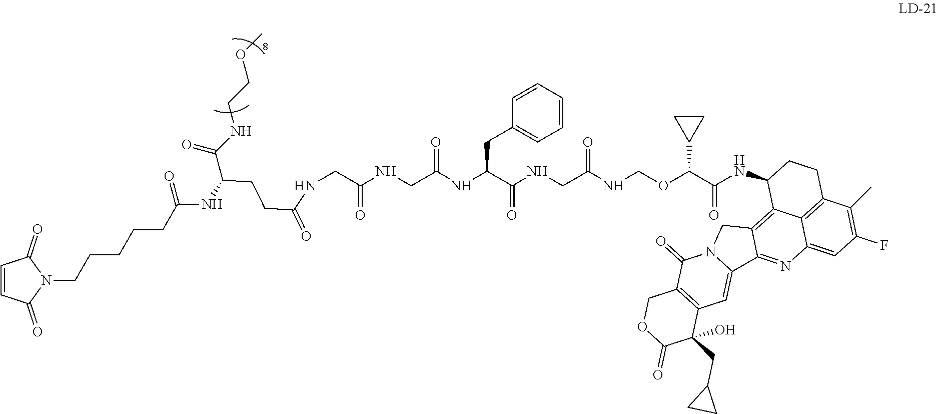

### Example 3-22: Preparation of LD-22

### (S)-N5-((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2, 5-dihydro-1H-pyrrol-1-yl)hexanamido)-N¹-methyl-N¹-((2S,3R,4R,5R)-2,3,4,5,6-pentahydroxyhexyl)pentanediamide LD-22

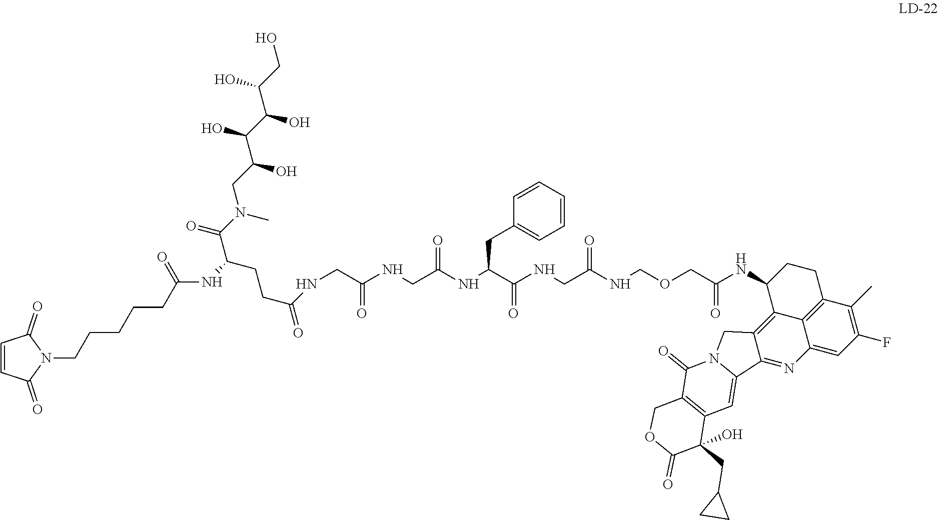 [SMILES: Cc1c(F)cc2nc3c(c4c2c1CC[C@@H]4NC(=O)COCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1]

### Example 3-23: Preparation of LD-23

### (S)-N5-((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-methyl-N-((2S,3R,4R,5R)-2,3,4,5,6-pentahydroxyhexyl)pentanediamide LD-23

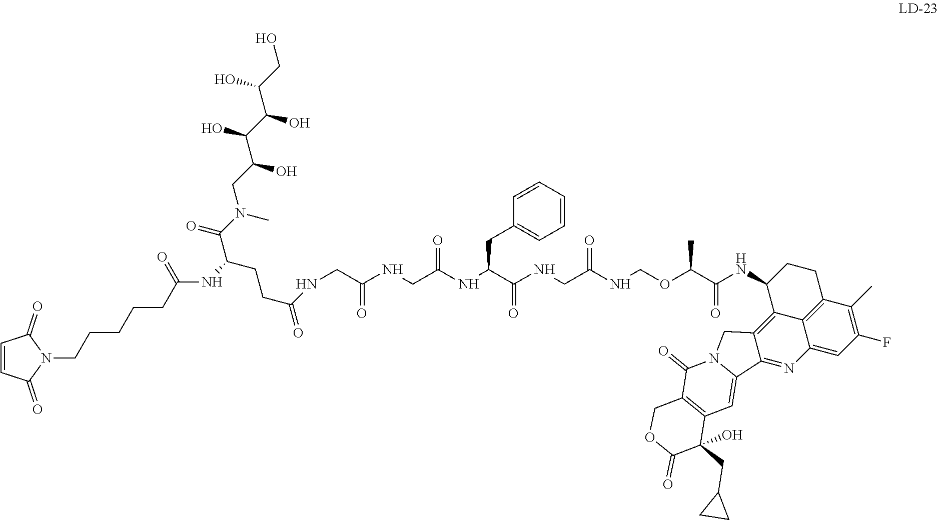 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1]

### Example 3-24: Preparation of LD-24

### (S)-N5-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-methyl-N-((2S,3R,4R,5R)-2,3,4,5,6-pentahydroxyhexyl)pentanediamide LD-24

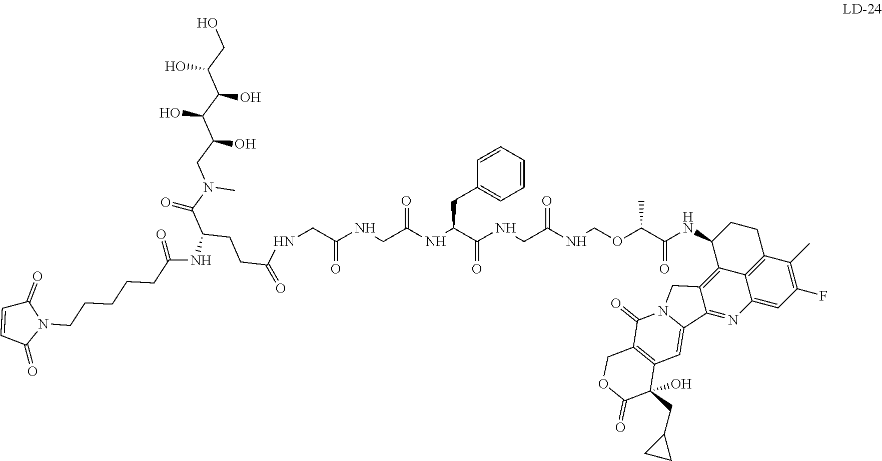 [SMILES: Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1]

### Example 3-25: Preparation of LD-25

### (S)-N5-((2S,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl) -2-(6-(2,5-dioxo-2,5-dihydro-1H-pyrrol-1-yl)hexanamido)-N-methyl-N¹-((2S,3R,4R,5R)-2,3,4,5,6-pentahydroxyhexyl)pentanediamide LD-25

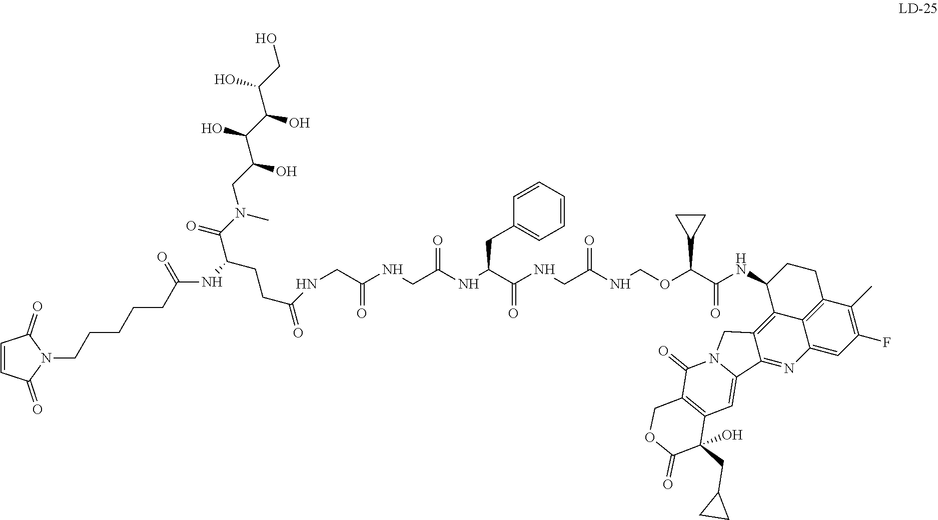 [SMILES: Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)Cn1c-3cc2c(c1=O)COC(=O)[C@]2(O)CC1CC1]

### Example 3-26: Preparation of LD-26

### (S)-N5-((2R,10S)-10-Benzyl-2-cyclopropyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-26

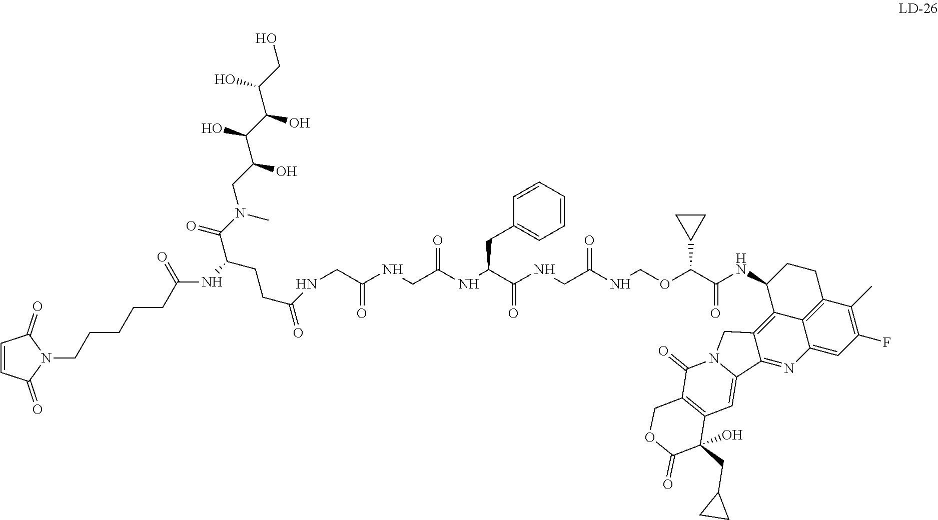 [SMILES: Cc1c(F)cc2nc3c(c4c2c1CC[C@@H]4NC(=O)[C@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)Cn1c-3cc2c(c1=O)COC(=O)[C@]2(O)CC1CC1]

### Example 3-27: Preparation of LD-27

### 2-(((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-27

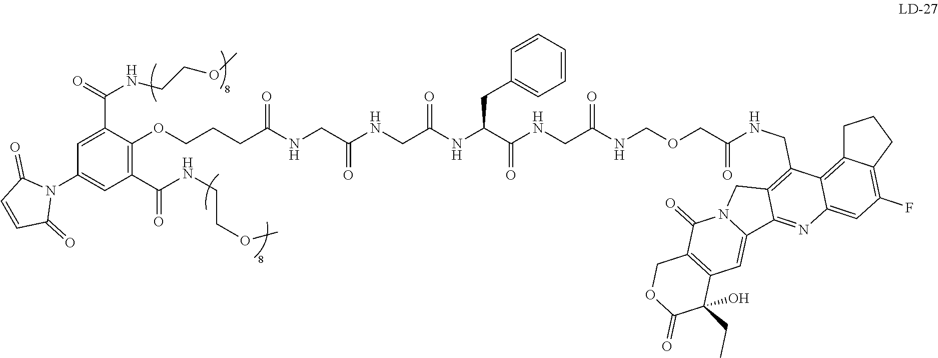

MS m/z (ESI): 959.5 [1/2(M+2)].

### Example 3-28: Preparation of LD-28

### 2-(((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-28

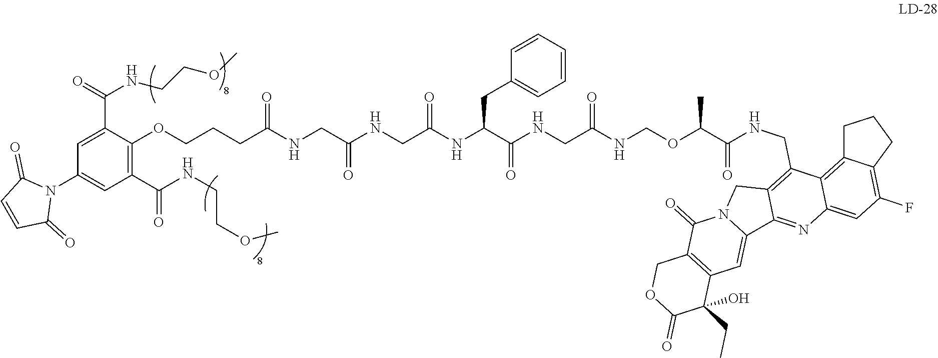

MS m/z (ESI): 966.4 [1/2(M+2)].

### Example 3-29: Preparation of LD-29

### 2-(((4S,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-29

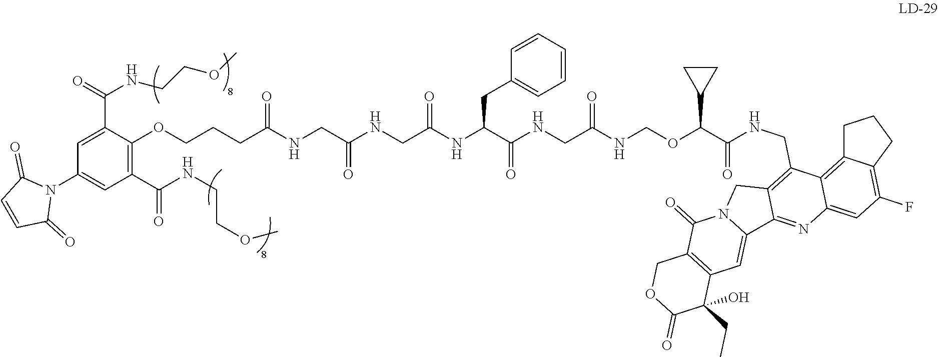

### Example 3-30: Preparation of LD-30

### 2-(((4R,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-30

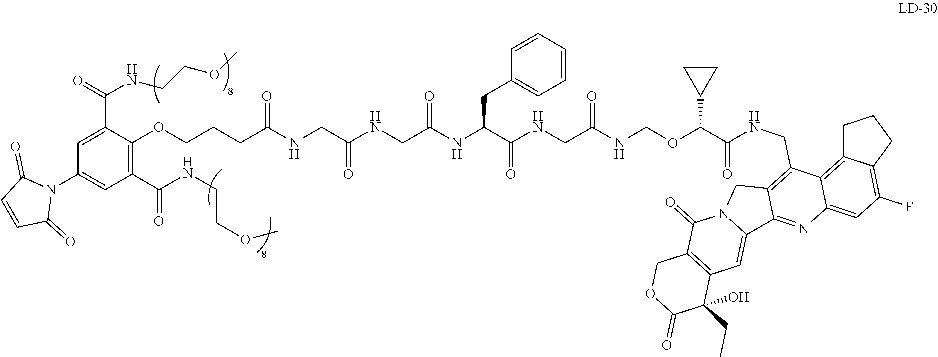

### Example 3-31: Preparation of LD-31

### 2-(((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-31

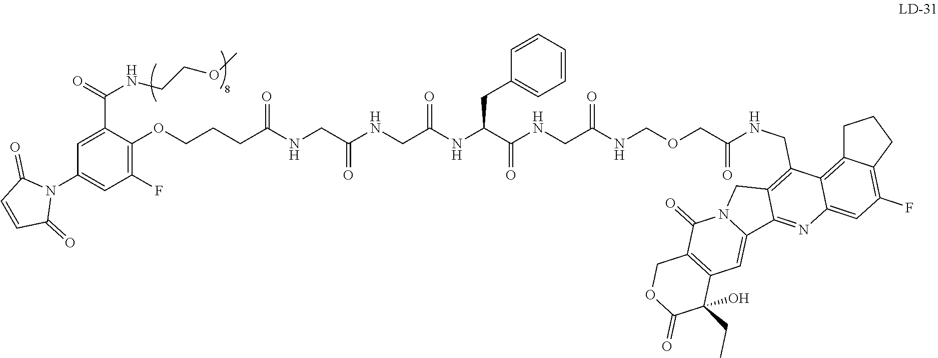

### Example 3-32: Preparation of LD-32

### 2-(((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-32

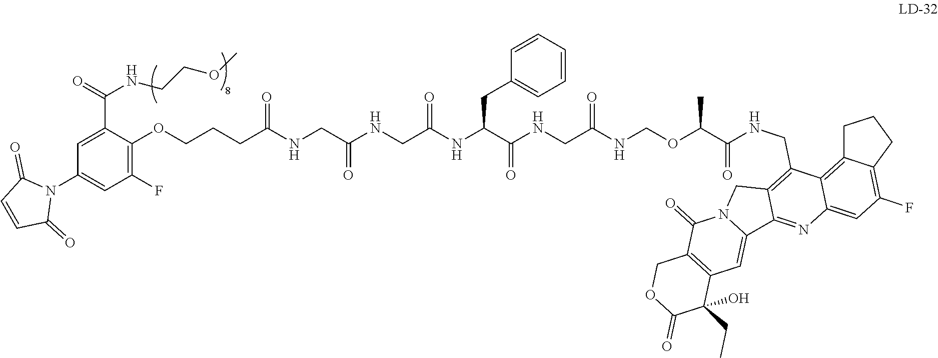

### Example 3-33: Preparation of LD-33

### 2-(((4S,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-33

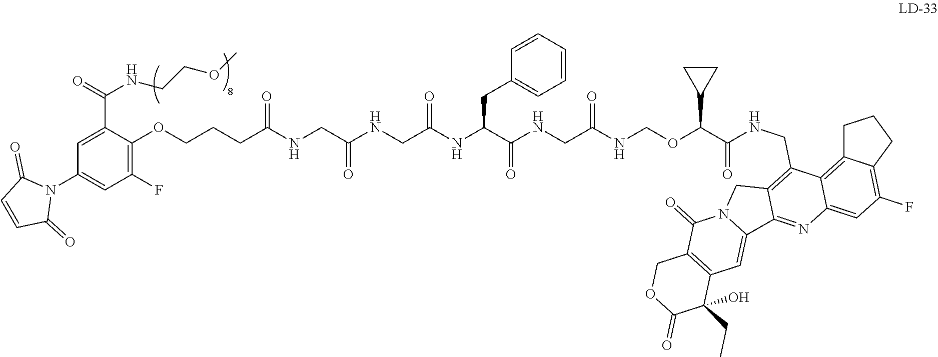

### Example 3-34: Preparation of LD-34

### 2-(((4R,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-34

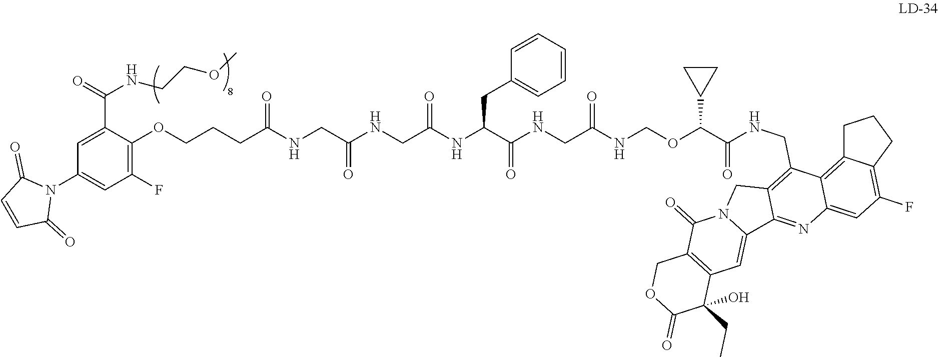

### Example 3-35: Preparation of LD-35

### (S)-N5-((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-35

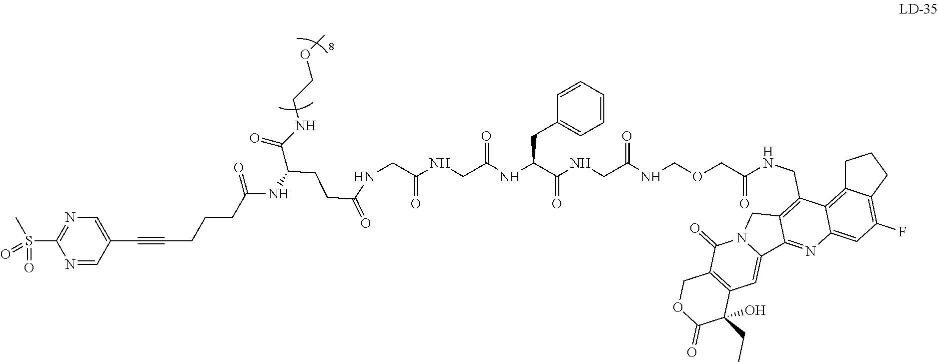

MS m/z (ESI): 793.9 [1/2(M+2)].

### Example 3-36: Preparation of LD-36

### (S)-N5-((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17-pentaoxo-5-oxa-2,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-36

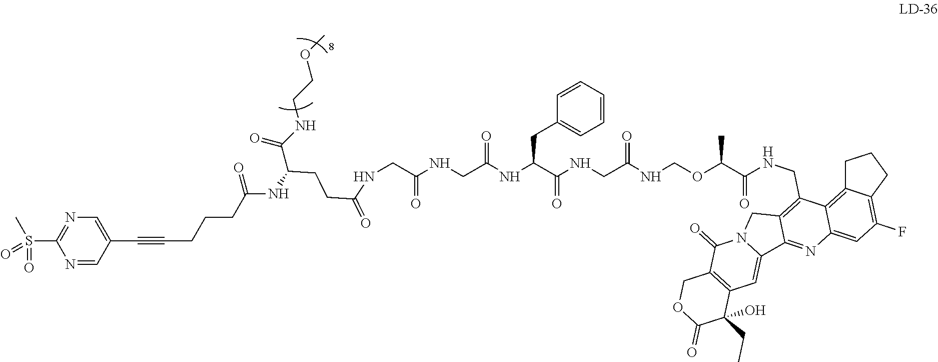

MS m/z (ESI): 800.8 [1/2(M+2)].

### Example 3-37: Preparation of LD-37

### (S)-N5-((4S,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2-(methylsulfonyl)pyri midin-5-yl)hex-5-ynamido)-N¹-(2,5,8,11,14,17,20,23-octaoxapentacosan-25-yl)pentanediamide LD-37

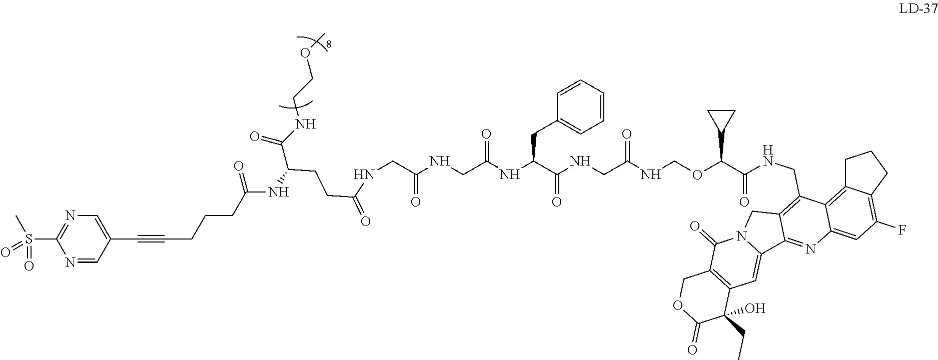

### Example 3-38: Preparation of LD-38

### (S)-N5-((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-38

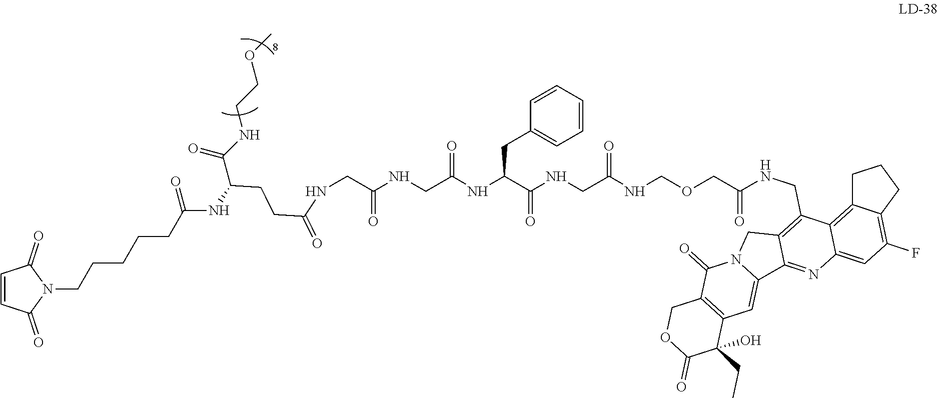

### Example 3-39: Preparation of LD-39

### (S)-N5-((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-39

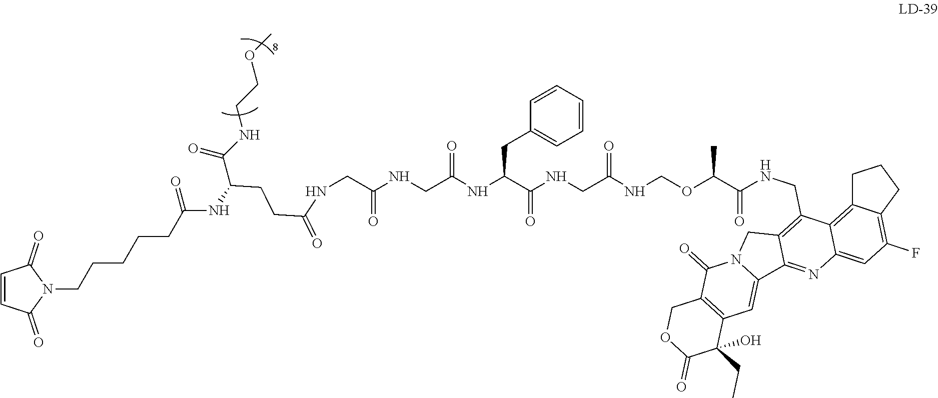

### Example 3-40: Preparation of LD-40

### (S)-N5-((4S,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-40

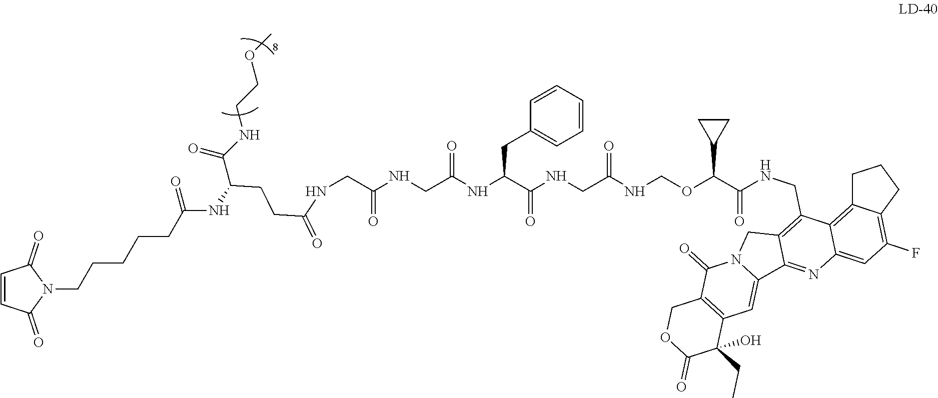

### Example 3-41: Preparation of LD-41

### (S)-N5-((4R,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentazaoctadecan-18-yl)-2-(6-(2,5-dioxy-2,5-dihydro-1H-pyrrol-1-yl)hexanamido)-N¹-(2,5,8,11,14,17,20,23-octaoxapentacosan-25-yl)pentanediamide LD-41

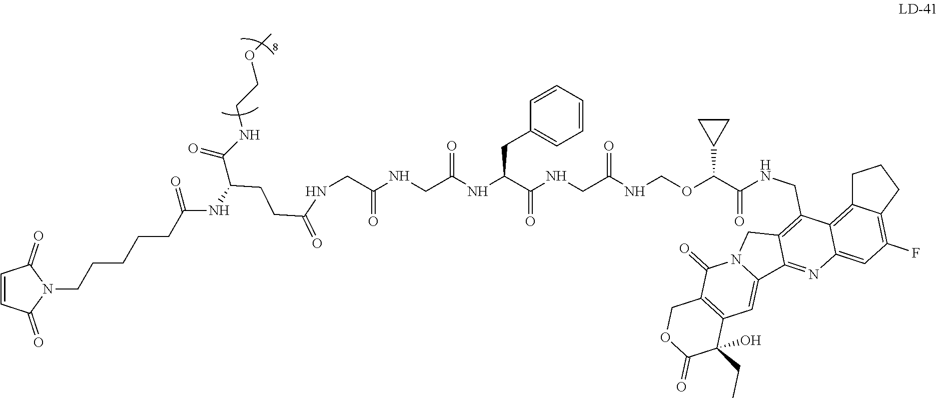

### Example 3-42: Preparation of LD-42

### (S)-N5-((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-42

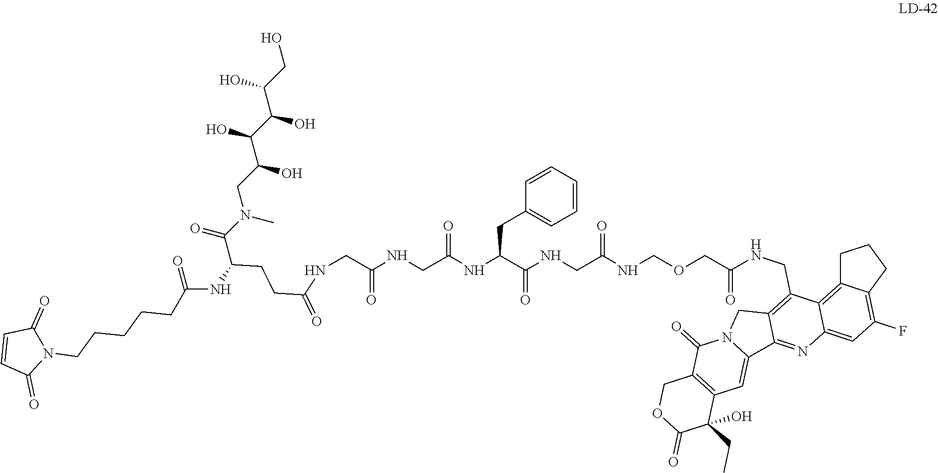 [SMILES: CC[C@@]1(O)C(=O)OCc2c1cc1n(c2=O)Cc2c-1nc1cc(F)c3c(c1c2CNC(=O)COCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)CCC3]

### Example 3-43: Preparation of LD-43

### (S)-N5-((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-43

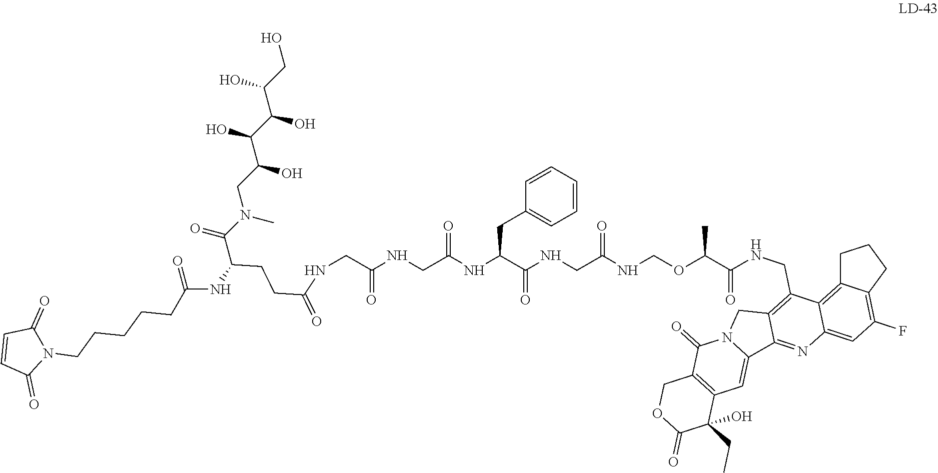 [SMILES: CC[C@@]1(O)C(=O)OCc2c1cc1n(c2=O)Cc2c-1nc1cc(F)c3c(c1c2CNC(=O)[C@H](C)OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)CCC3]

### Examples 3-44: LD-44

### (S)-N5-((4S,12S)-12-Benzyl-4-cyclopropyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-44

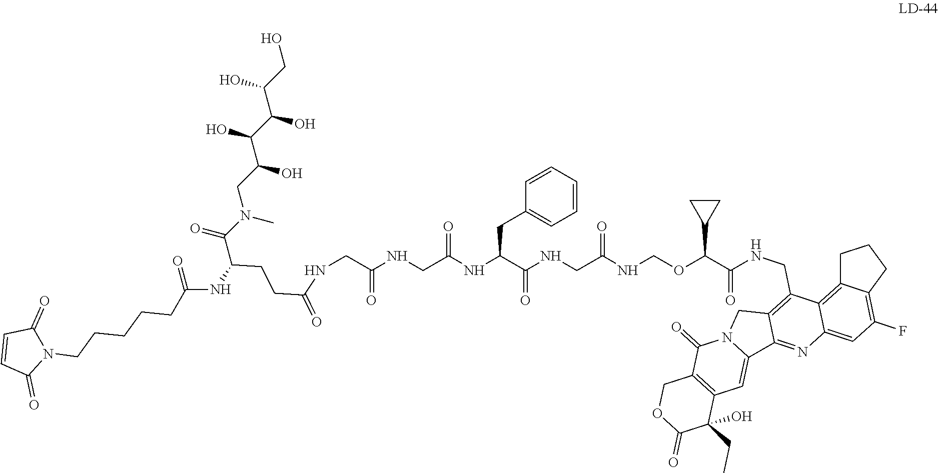 [SMILES: CC[C@@]1(O)C(=O)OCc2c1cc1n(c2=O)Cc2c-1nc1cc(F)c3c(c1c2CNC(=O)[C@@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)CCC3]

### Example 3-45: LD-45

### 2-(((2S,13S)-13-Benzyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-45

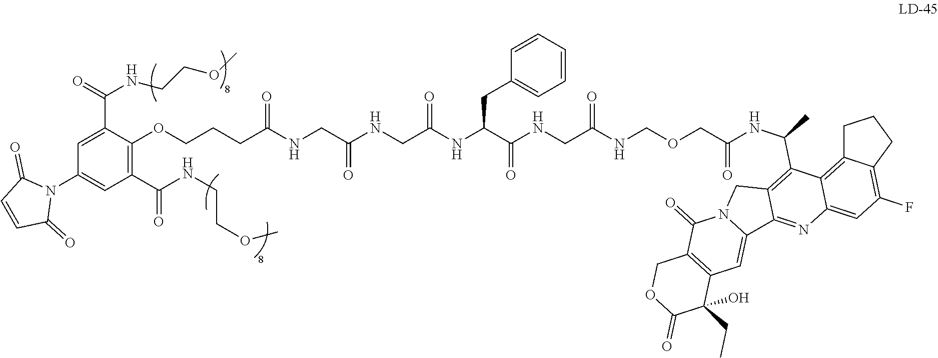

### Example 3-46: LD-46

### 2-(((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-46

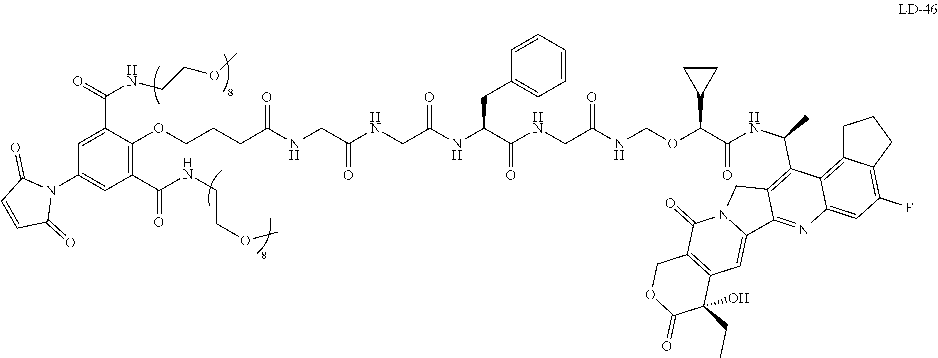

### Example 3-47: LD-47

### 2-(((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-47

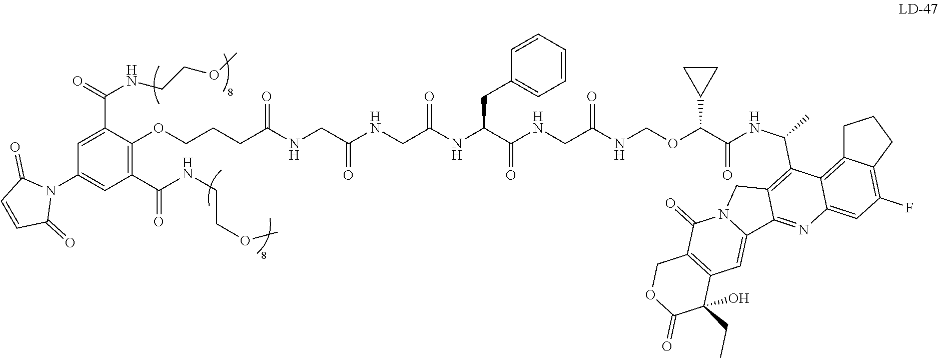

### Example 3-48: LD-48

### 2-(((2S,13S)-13-Benzyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-48

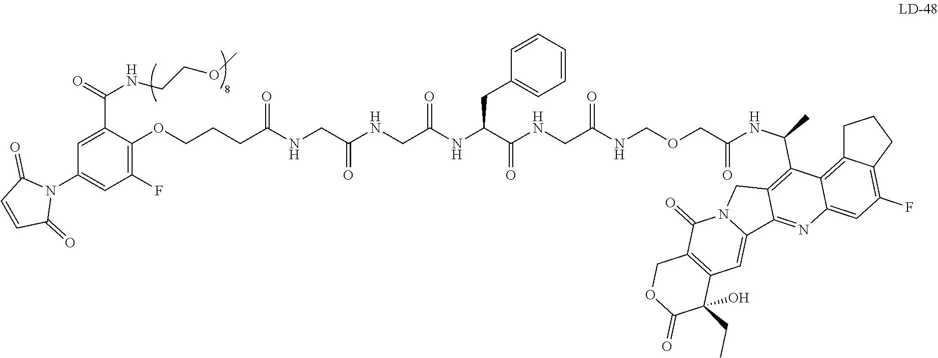

### Example 3-49: LD-49

### 2-(((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-49

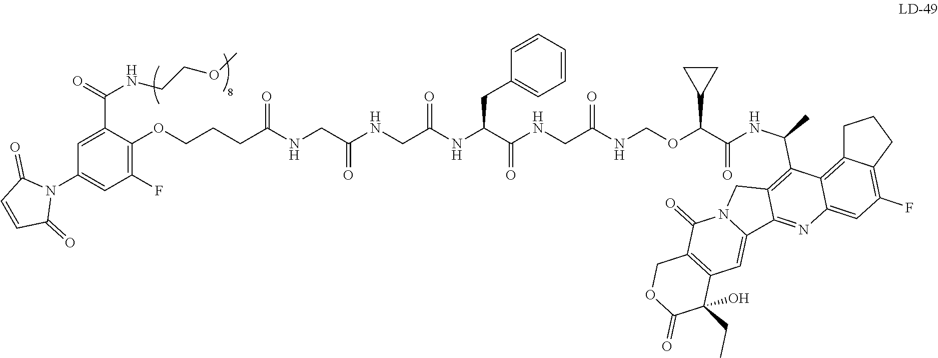

### Example 3-50: LD-50

### 2-(((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18,21-hexaoxo-6-oxa-3,8,11,14,17,20-hexaazatetracosan-24-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-50

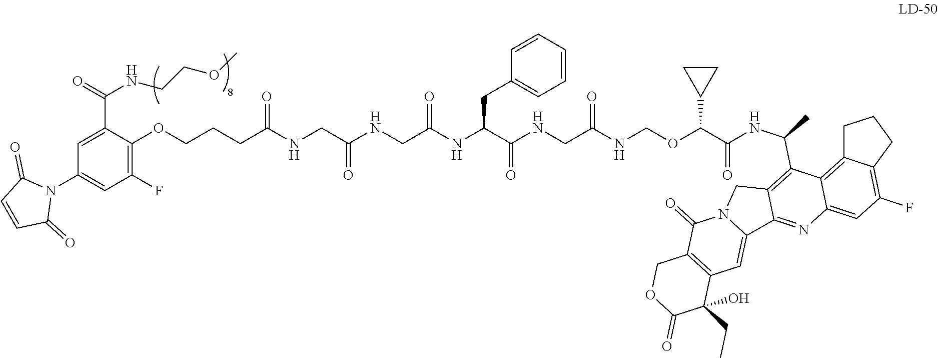

### Example 3-51: LD-51

### (S)-N5-((2S,13S)-13-Benzyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2-(methylsulfonyl)pyrimidin-5-yl)he x-5-ynamido)-N¹-(2,5,8,11,14,17,20,23-octaoxapentacosan-25-yl)pentanediamide LD-51

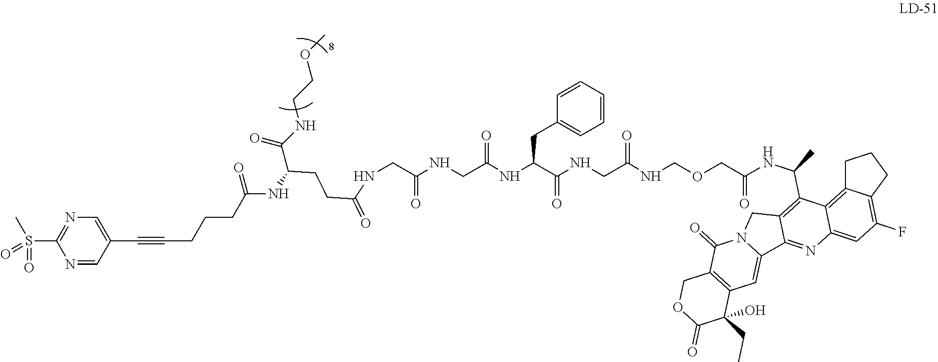

### Example 3-52: LD-52

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-52

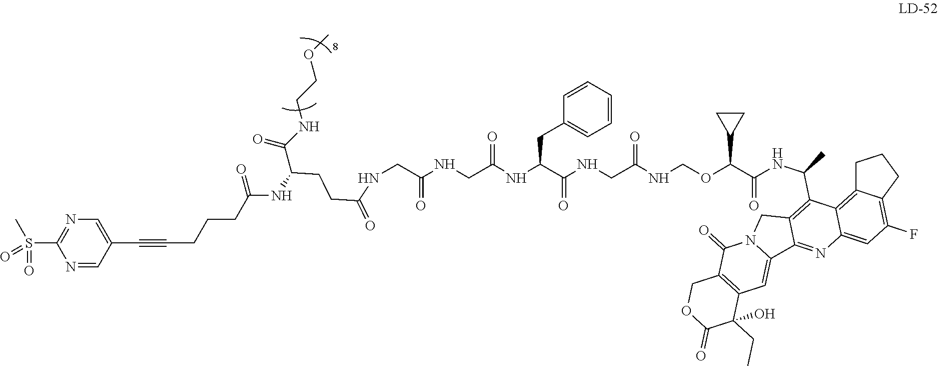

### Example 3-53: LD-53

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-53

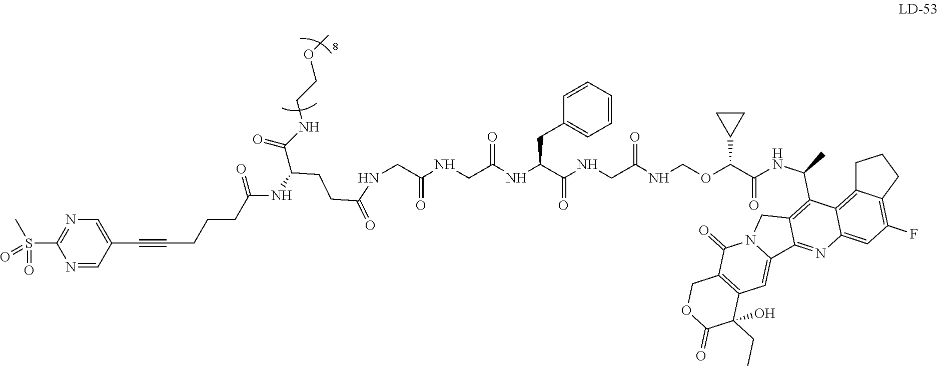

### Example 3-54: LD-54

### (S)-N5-((2S,13S)-13-Benzyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-54

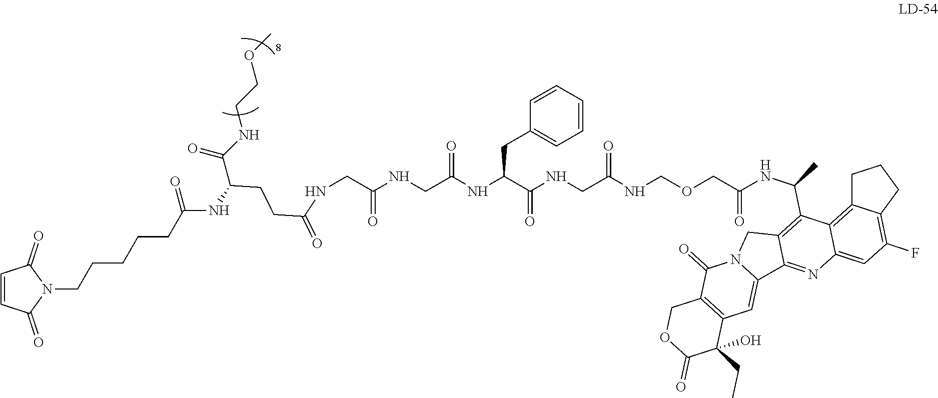

### Example 3-55: LD-55

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihy dro-1H-pyrrol-1-yl)hexanamido)-N¹-(2,5,8,11,14,17,20,23-octaoxapentacosan-25-yl)pentanediamide LD-55

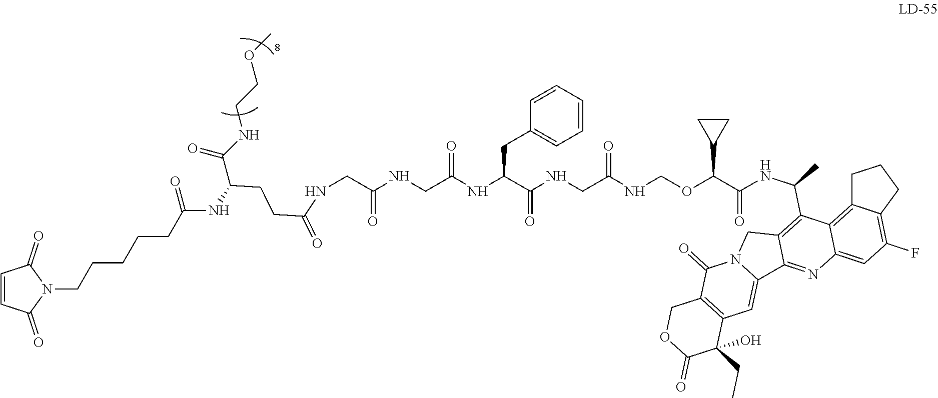

### Example 3-56: LD-56

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihy dro-1H-pyrrol-1-yl)hexanamido)-N¹-(2,5,8,11,14,17,20,23-octaoxapentacosan-25-yl)pentanediamide LD-56

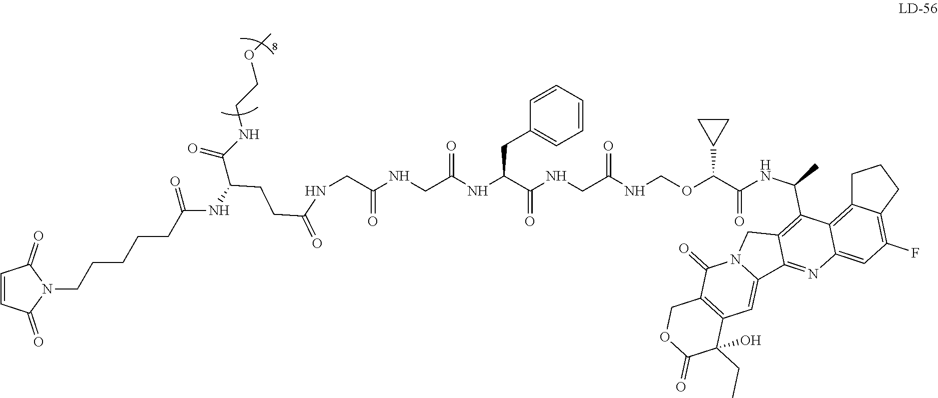

### Example 3-57: LD-57

### (S)-N5-((2S,13S)-13-Benzyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-57

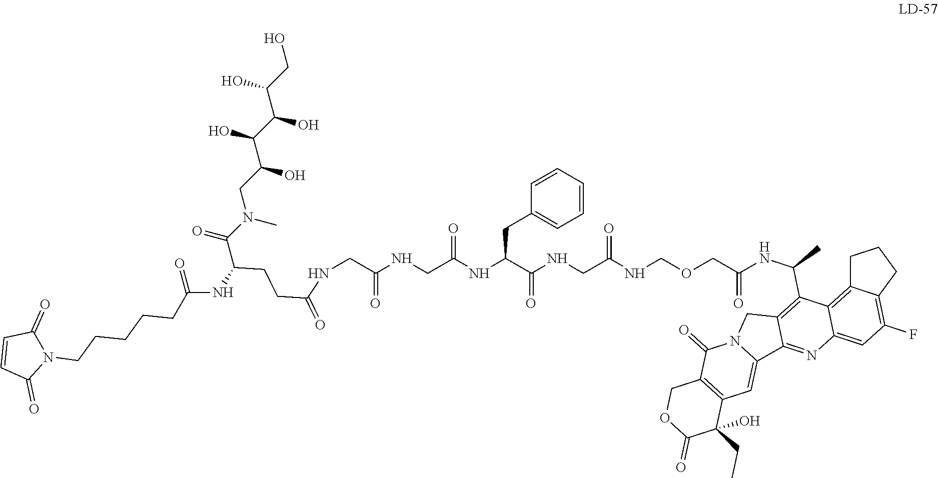 [SMILES: CC[C@@]1(O)C(=O)OCc2c1cc1n(c2=O)Cc2c-1nc1cc(F)c3c(c1c2[C@H](C)NC(=O)COCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)CCC3]

### Example 3-58: LD-58

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihy dro-1H-pyrrol-1-yl)hexanamido)-N¹-methyl-N¹-((2S,3R,4R,5R)-2,3,4,5,6-pentahydroxyhexyl)pentanediamide LD-58

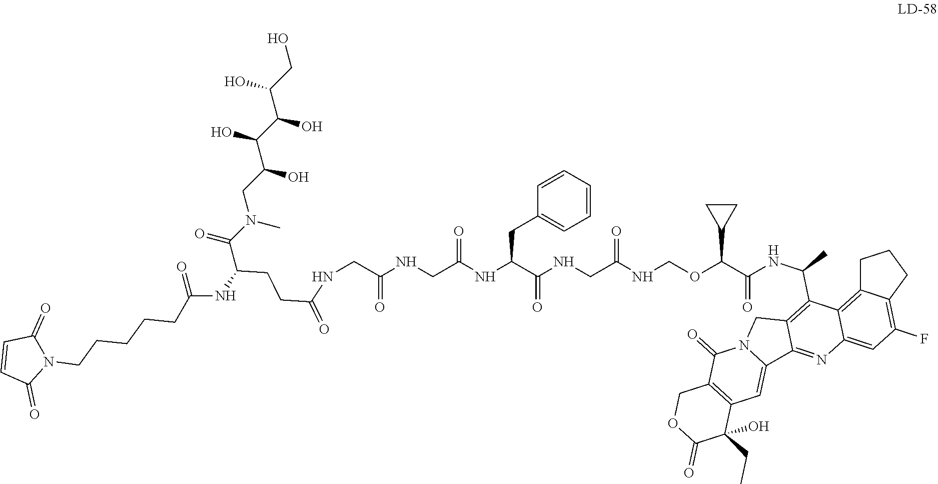 [SMILES: CC[C@@]1(O)C(=O)OCc2c1cc1n(c2=O)Cc2c-1nc1cc(F)c3c(c1c2[C@H](C)NC(=O)[C@@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)CCC3]

### Example 3-59: LD-59

### (S)-N5-((2S,5R,13S)-13-Benzyl-5-cyclopropyl-2-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4,9,12,15,18-pentaoxo-6-oxa-3,8,11,14,17-pentaazanonadecan-19-yl)-2-(6-(2,5-dioxo-2,5-dihy dro-1H-pyrrol-1-yl)hexanamido)-N¹-methyl-N¹-((2S,3R,4R,5R)-2,3,4,5,6-pentahydroxyhexyl)pentanediamide LD-59

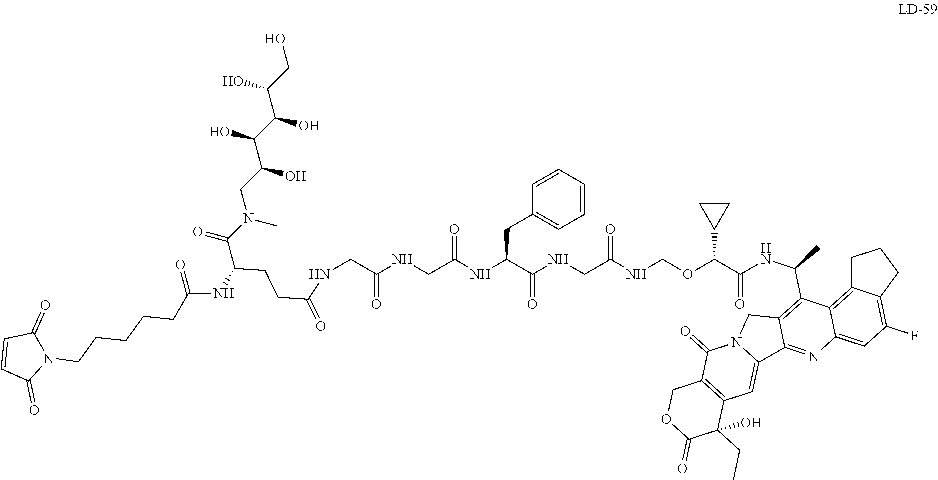 [SMILES: CC[C@@]1(O)C(=O)OCc2c1cc1n(c2=O)Cc2c-1nc1cc(F)c3c(c1c2[C@H](C)NC(=O)[C@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)CCC3]

### Example 3-60: LD-60

### 2-(((S)-12-Benzyl-1-((S)-8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-60

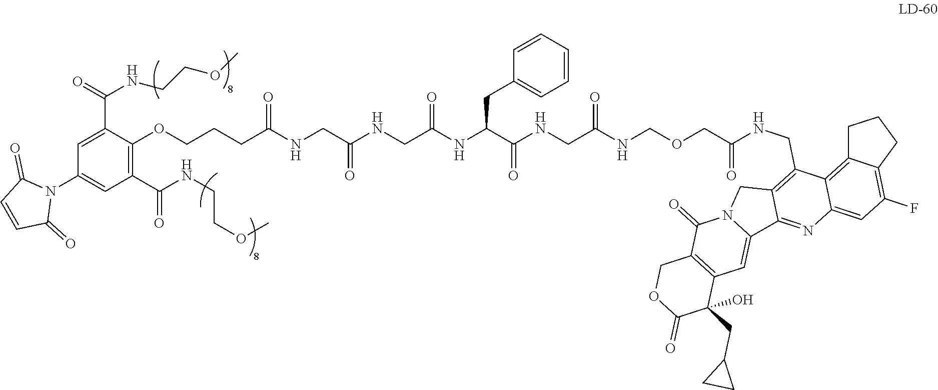

### Example 3-61: LD-61

### 2-(((S)-12-Benzyl-1-((S)-8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-61

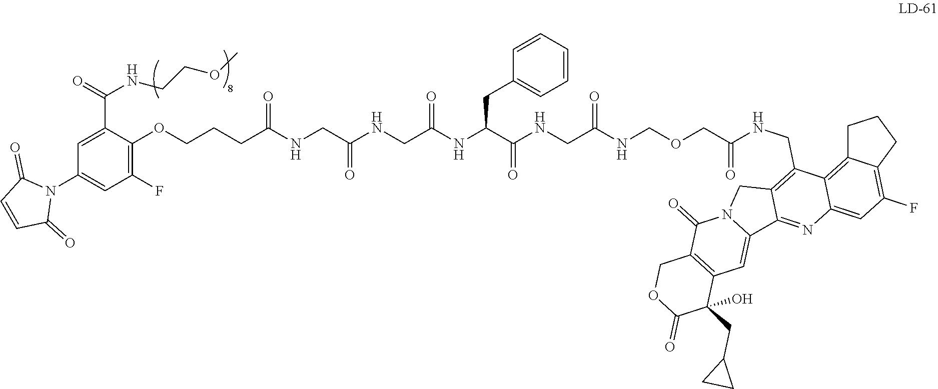

### Example 3-62: LD-62

### (S)-N5-((S)-12-Benzyl-1-((S)-8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-62

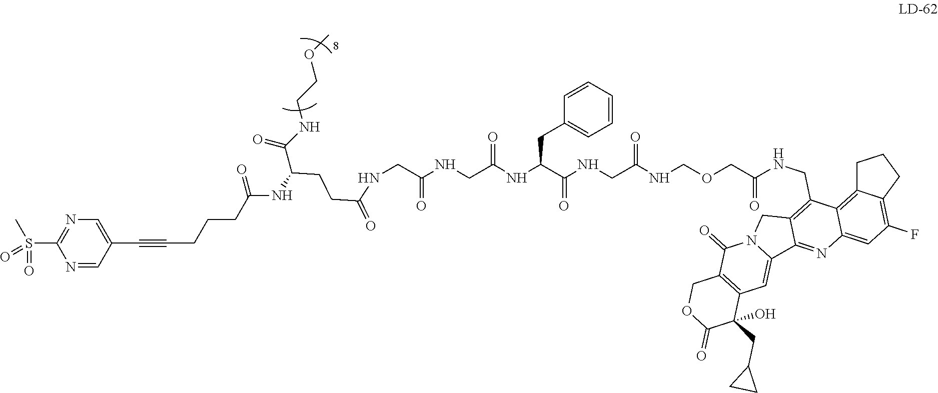

### Example 3-63: LD-63

### (S)-N5-((S)-12-Benzyl-1-((S)-8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-63

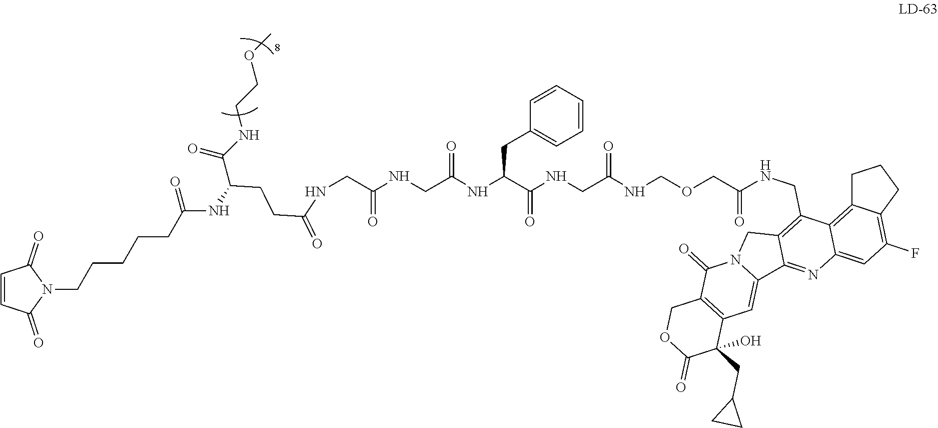

### Example 3-64: LD-64

### (S)-N5-((S)-12-Benzyl-1-((S)-8-(cyclopropylmethyl)-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-64

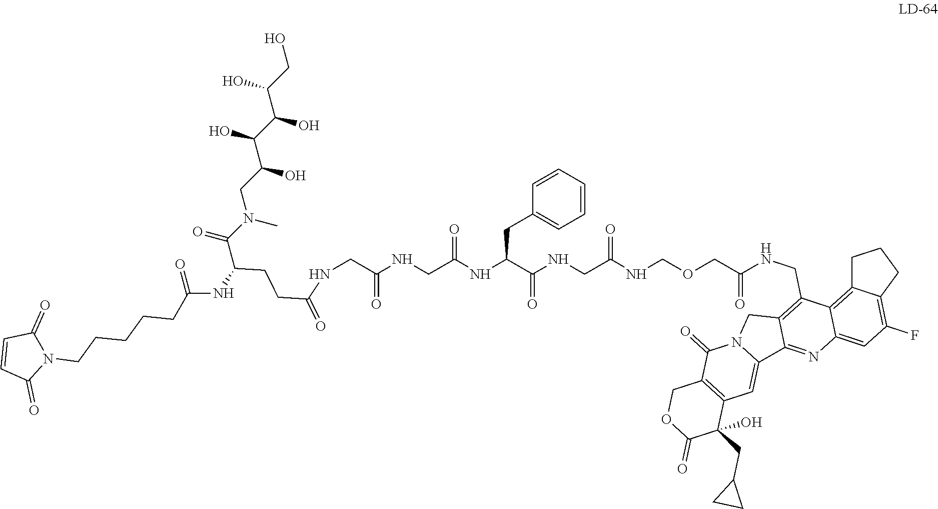 [SMILES: CN(C[C@H](O)[C@@H](O)[C@H](O)[C@H](O)CO)C(=O)[C@H](CCC(=O)NCC(=O)NCC(=O)N[C@@H](Cc1ccccc1)C(=O)NCC(=O)NCOCC(=O)NCc1c2c(nc3cc(F)c4c(c13)CCC4)-c1cc3c(c(=O)n1C2)COC(=O)[C@]3(O)CC1CC1)NC(=O)CCCCCN1C(=O)C=CC1=O]

### Example 3-65: LD-65

### 2-(((S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-65

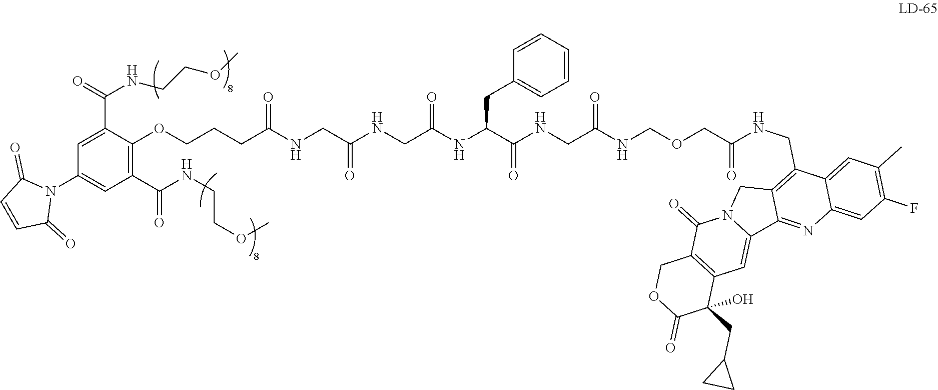

### Example 3-66: LD-66

### 2-(((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-66

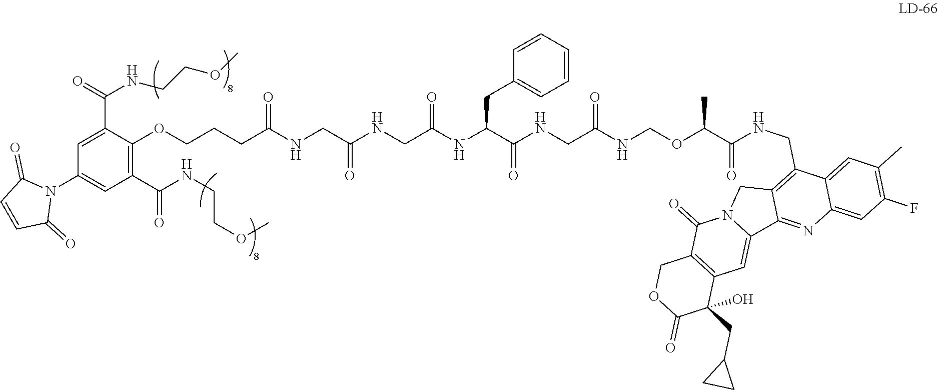

### Example 3-67: LD-67

### 2-(((4R,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-67

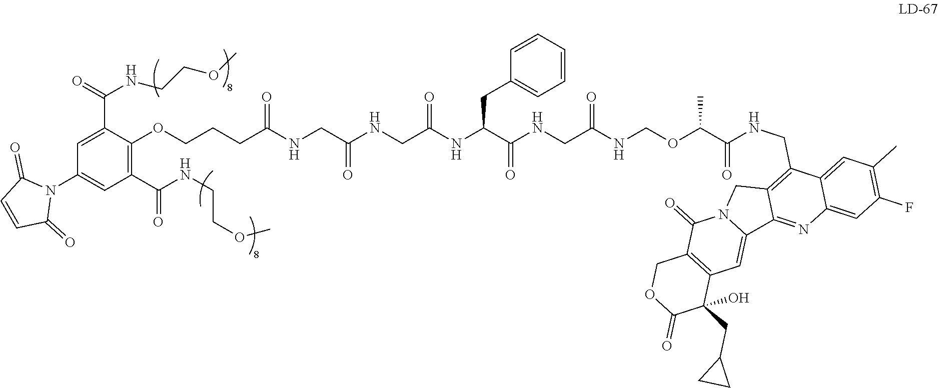

### Example 3-68: LD-68

### 2-(((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-68

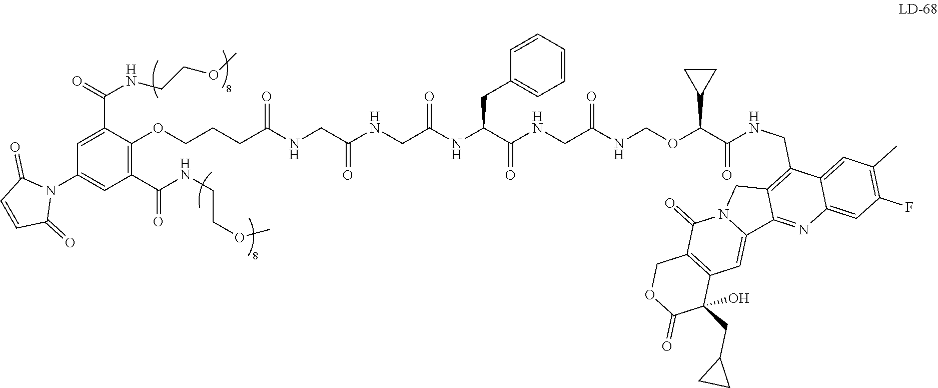

### Example 3-69: LD-69

### 2-(((4R,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-N¹,N³-di(2,5,8,11,14,17,20,23-octaoxapentacosan-25-yl)isophthalamide LD-69

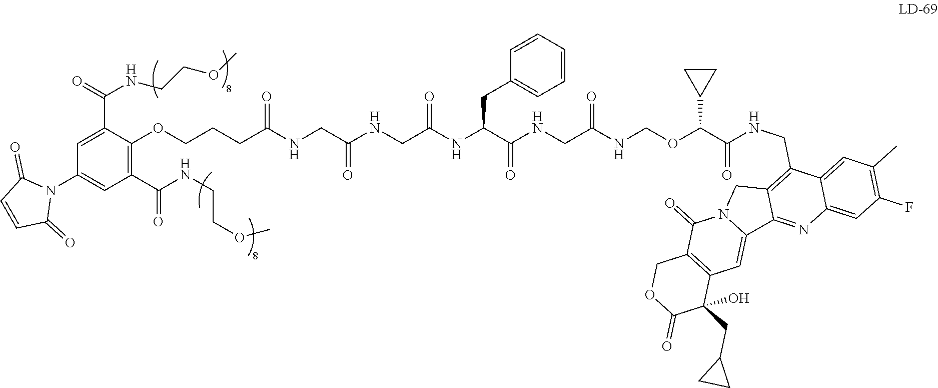

### Example 3-70: LD-70

### 2-(((S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-70

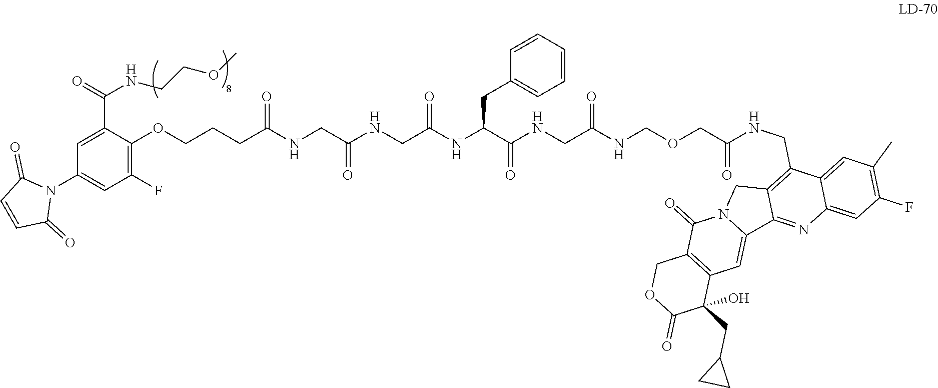

### Example 3-71: LD-71

### 2-(((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-71

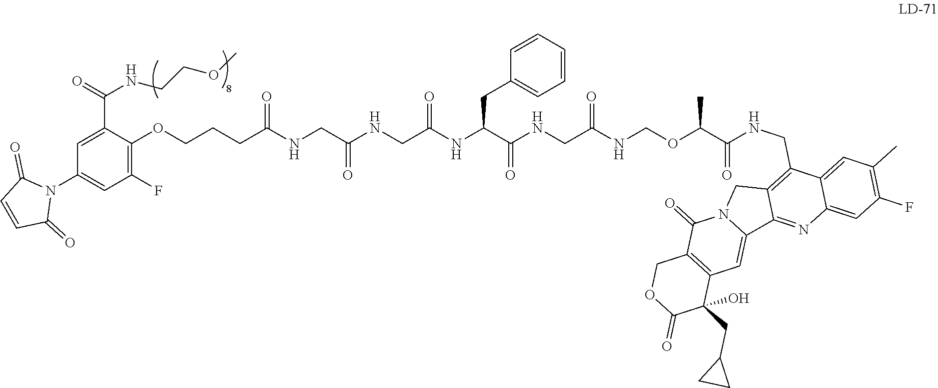

### Example 3-72: LD-72

### 2-(((4R,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-72

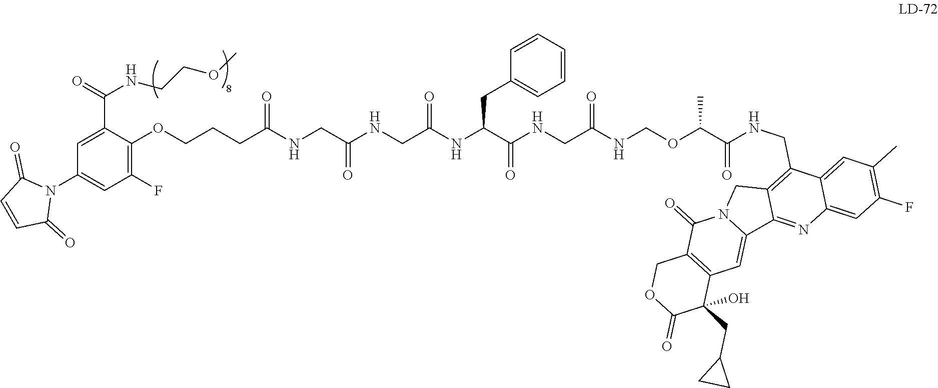

### Example 3-73: LD-73

### 2-(((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-73

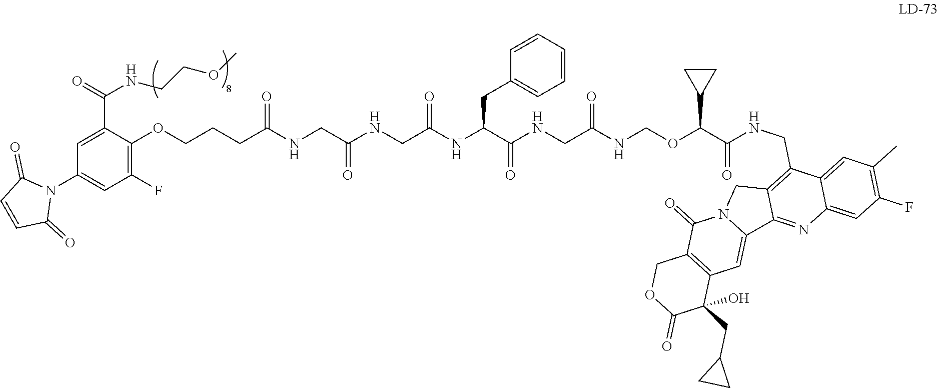

### Example 3-74: LD-74

### 2-(((4R,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazatricosan-23-yl)oxy)-5-(2,5-dioxo-2,5-dihydro-1H-pyrrol-1-yl)-3-fluoro-N-(2,5,8,11,14,17,20,23-octaoxapentacosan-25-yl)benzamide LD-74

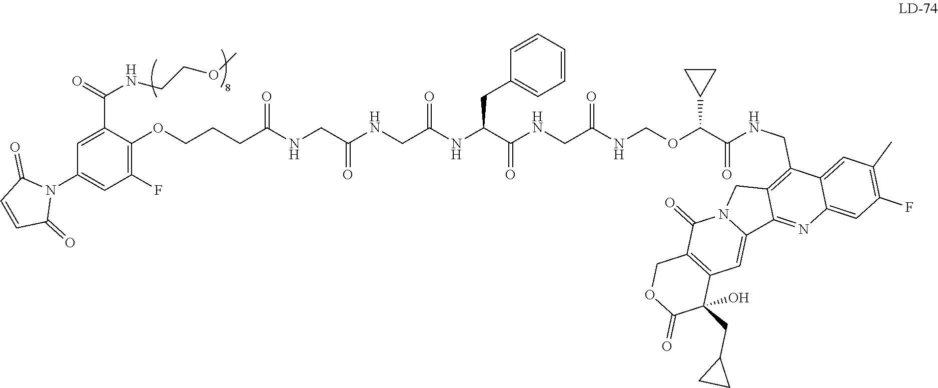

### Example 3-75: LD-75

### (S)-N5-((S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-75

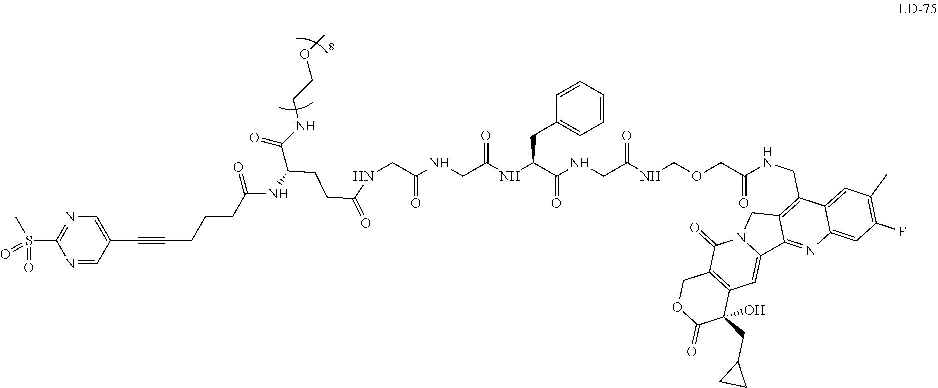

### Example 3-76: LD-76

### (S)-N5-((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-76

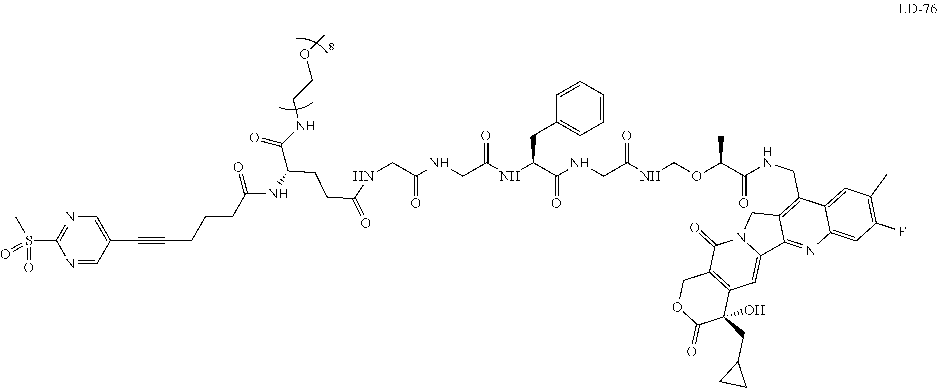

### Example 3-77: LD-77

### (S)-N5-((4R,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2-(methylsulfonyl)pyrimidin-5-yl)hex-5-ynamido)-N¹-(2,5,8,11,14,17,20,23-octaoxapentacosan-25-yl)pentanediamide LD-77

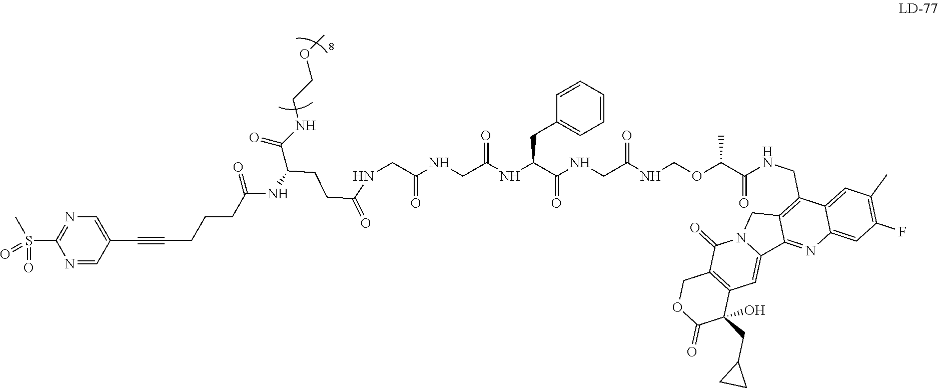

### Example 3-78: LD-78

### (S)-N5-((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2-(methylsulfonyl)p yrimidin-5-yl)hex-5-ynamido)-N¹-(2,5,8,11,14,17,20,23-octaoxapentacosan-25-yl)pentanediamide LD-78

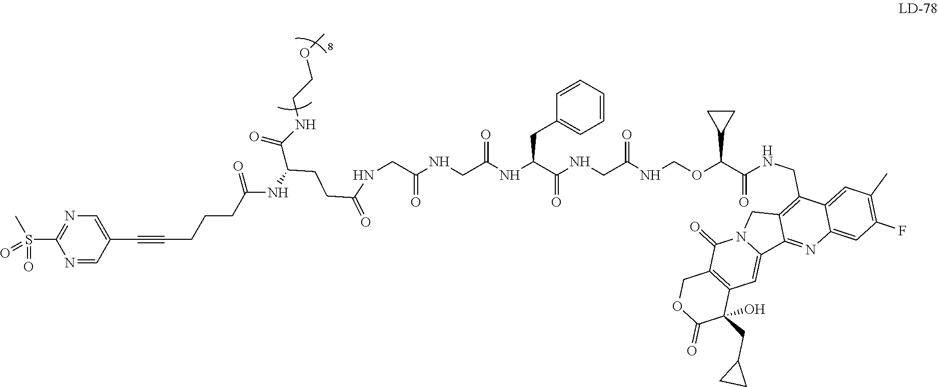

### Example 3-79: LD-79

### (S)-N5-((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2-(methylsulfonyl)p yrimidin-5-yl)hex-5-ynamido)-N¹-(2,5,8,11,14,17,20,23-octaoxapentacosan-25-yl)pentanediamide LD-79

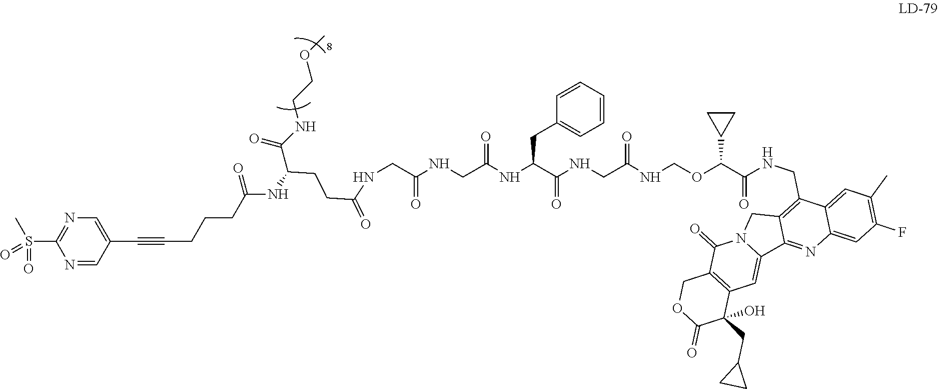

### Example 3-80: LD-80

### (S)-N5-((S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-penta oxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-80

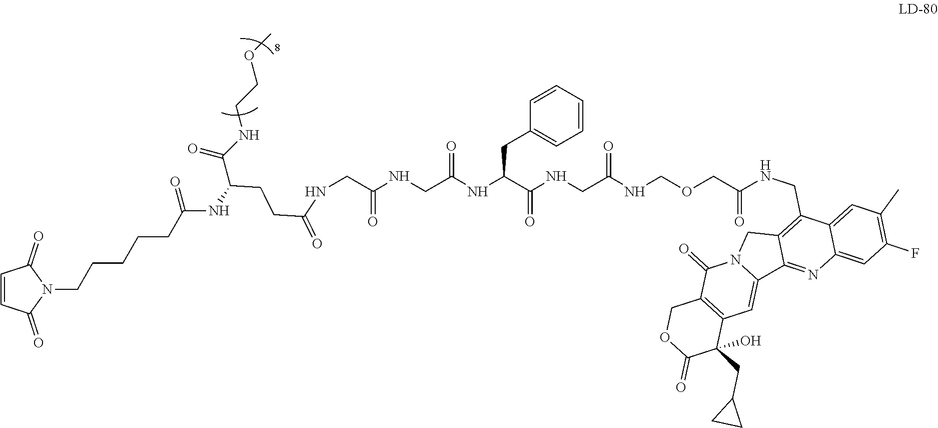

### Example 3-81: LD-81

### (S)-N5-((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-81

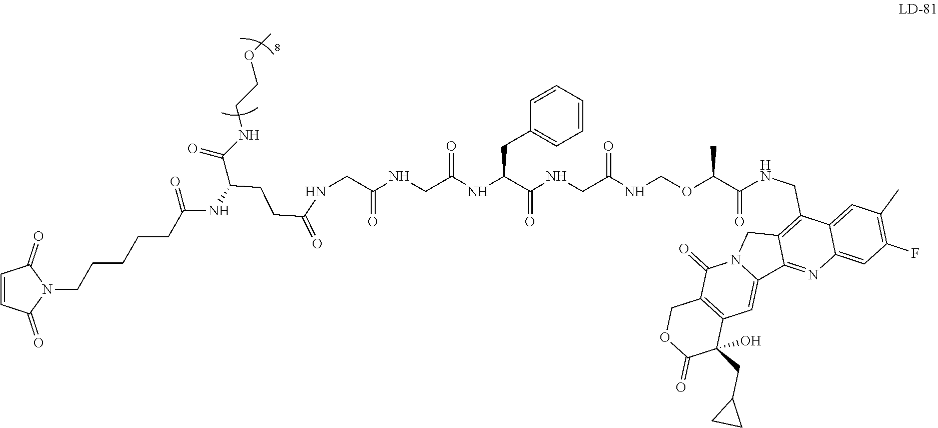

### Example 3-82: LD-82

### (S)-N5-((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-82

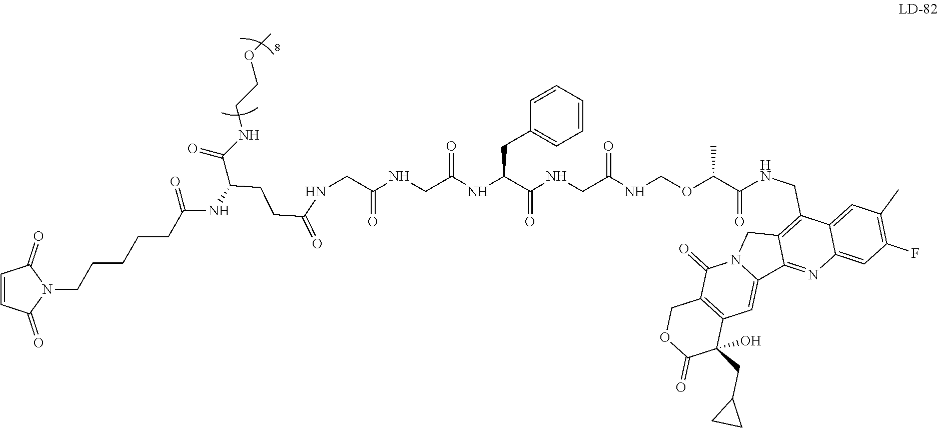

### Example 3-83: LD-83

### (S)-N5-((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2,5-dioxo-2,5-dihyd ro-1H-pyrrol-1-yl)hexanamido)-N¹-(2,5,8,11,14,17,20,23-octaoxapentacosan-25-yl)pentanediamide LD-83

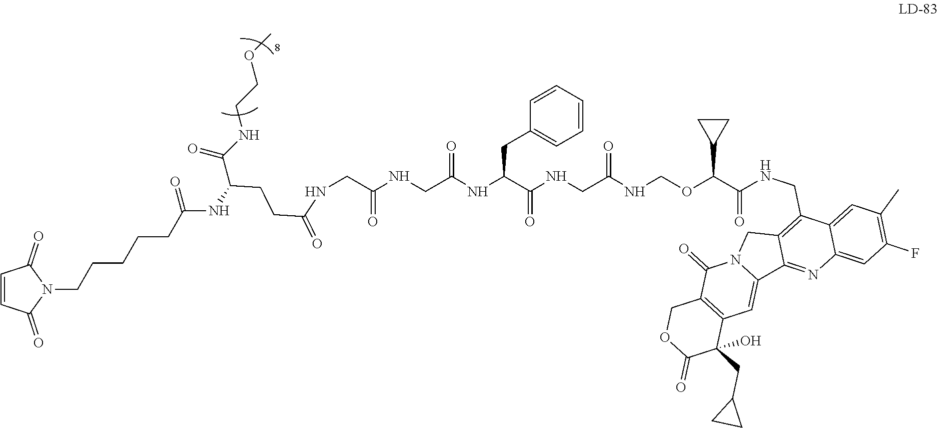

### Example 3-84: LD-84

### (S)-N5-((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2,5-dioxo-2,5-dihyd ro-1H-pyrrol-1-yl)hexanamido)-N¹-(2,5,8,11,14,17,20,23-octaoxapentacosan-25-yl)pentanediamide LD-84

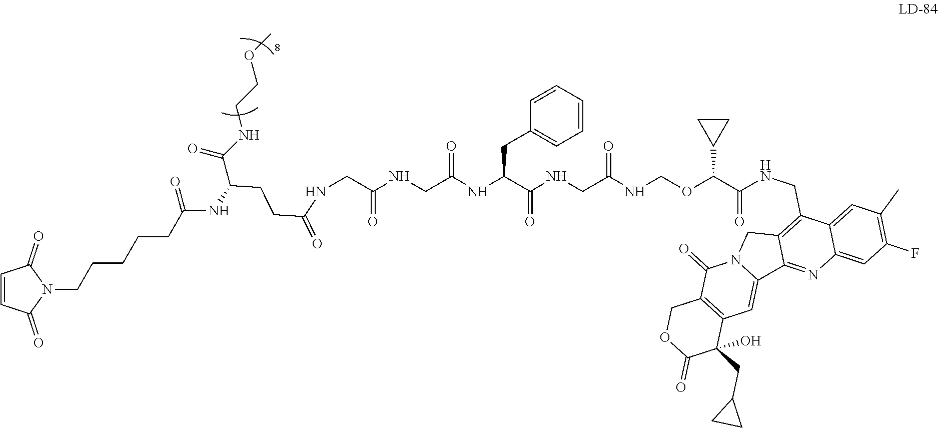

### Example 3-85: LD-85

### (S)-N5-((S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2,5-dioxo-2,5-dihydro-1H-pyrrol-1-yl) hexanamido)-N¹-methyl-N¹-((2S,3R,4R,5R)-2,3,4,5,6-pentahydroxyhexyl)pentanediamide LD-85

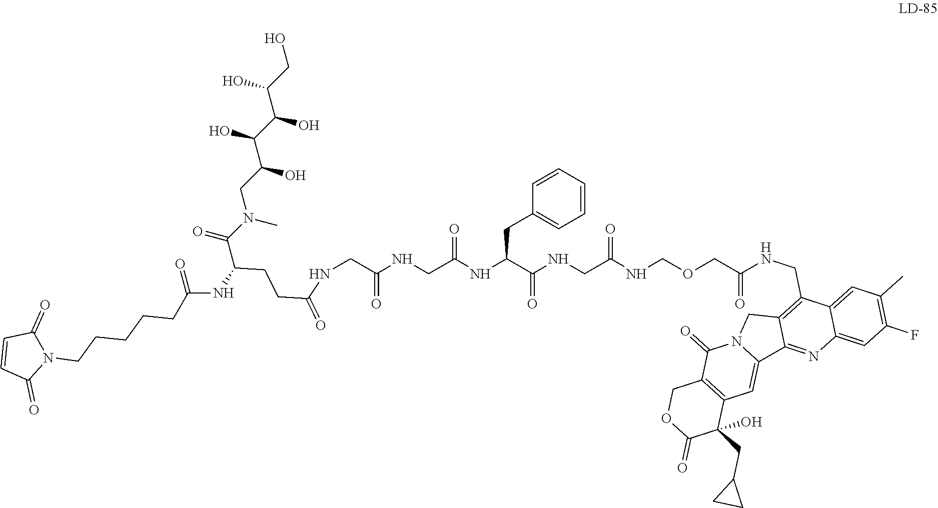 [SMILES: Cc1cc2c(CNC(=O)COCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1]

### Example 3-86 LD-86

### (S)-N5-((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-86

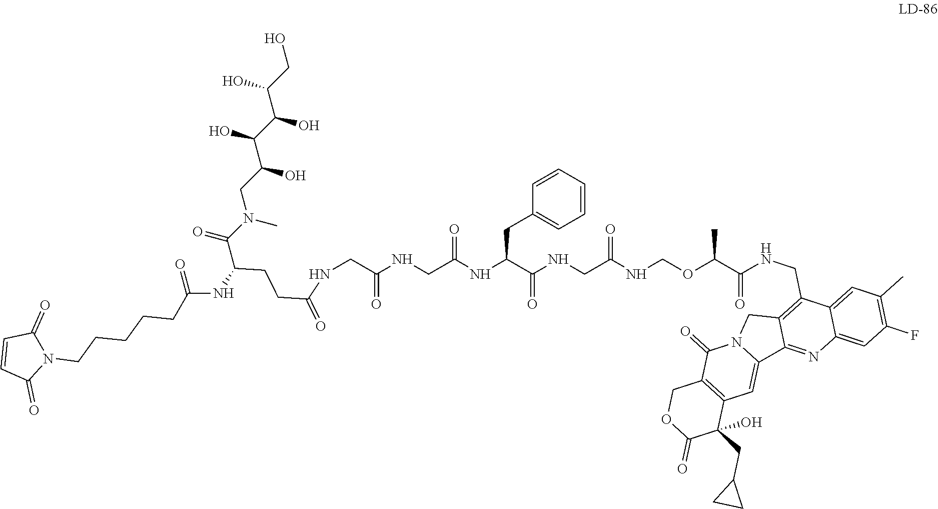 [SMILES: Cc1cc2c(CNC(=O)[C@H](C)OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1]

### Example 3-87: LD-87

### (S)-N5-((4S,12S)-12-Benzyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-4-methyl-3,8,11,14,17-pentaoxo-5-oxa-2,7,10,13,16-pentaazaoctadecan-18-yl)-2-(6-(2,5-dioxo-2,5-dihydro-1H-pyrrol-1-yl)hexanamido)-N¹-methyl-N¹-((2S,3R,4R,5R)-2,3,4,5,6-pentahydroxyhexyl)pentanediamide LD-87

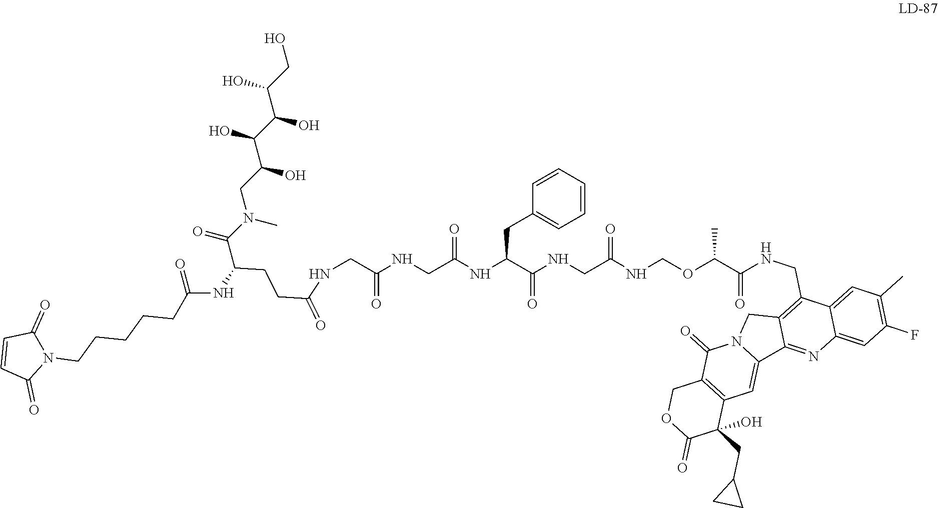 [SMILES: Cc1cc2c(CNC(=O)[C@@H](C)OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1]

### Example 3-88: LD-88

### (S)-N5-((4S,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2,5-dioxo-2,5-dihyd ro-1H-pyrrol-1-yl)hexanamido)-N¹-methyl-N¹-((2S,3R,4R,5R)-2,3,4,5,6-pentahydroxyhexyl)pentanediamide LD-88

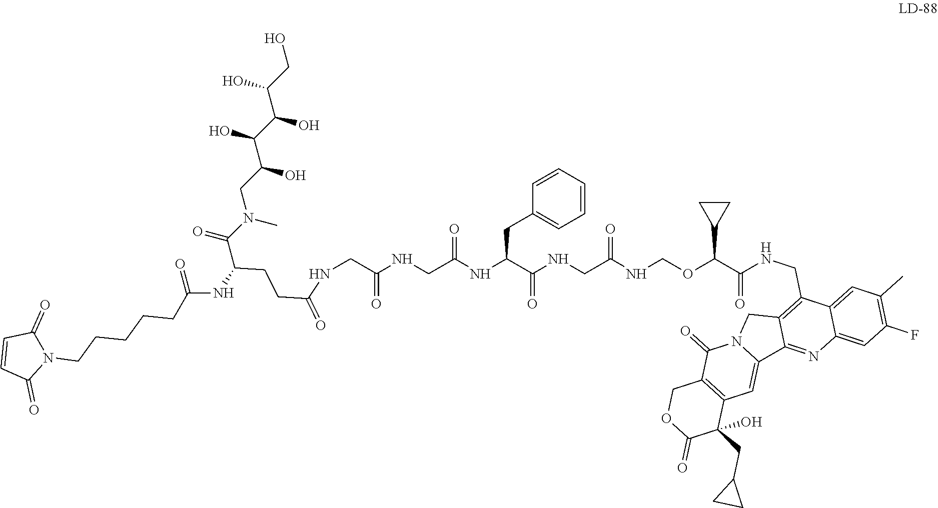 [SMILES: Cc1cc2c(CNC(=O)[C@@H](OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)C3CC3)c3c(nc2cc1F)-c1cc2c(c(=O)n1C3)COC(=O)[C@]2(O)CC1CC1]

### Example 3-89: LD-89

### (S)-N5-((4R,12S)-12-Benzyl-4-cyclopropyl-1-((S)-4-(cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-2-(6-(2,5-dioxo-2,5-dihyd ro-1H-pyrrol-1-yl)hexanamido)-N¹-methyl-N¹-((2S,3R,4R,5R)-2,3,4,5,6-pentahydroxyhexyl)pentanediamide LD-89

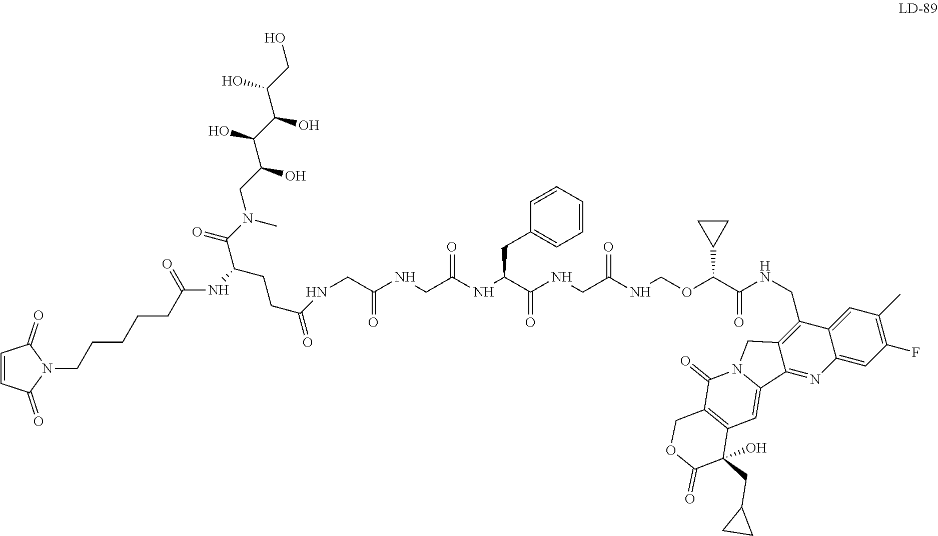 [SMILES: Cc1cc2c(CNC(=O)[C@H](OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)C3CC3)c3c(nc2cc1F)-c1cc2c(c(=O)n1C3)COC(=O)[C@]2(O)CC1CC1]

### Example 3-90: LD-90

### 5-(2,5-Dioxo-2,5-dihydro-1H-pyrrol-1-yl)-2-(((9S,12S,15S)-1-((S)-8-ethyl-4-fluoro-8-hydroxy-9,12-dioxo-2,3,8,9,12,14-hexahydro-11H,11H-cyclopenta[f]pyrano[3′,4′:6,7]indolizino[1,2-b]quinolin-15-yl)-9,12,15-trimethyl-3,8,11,14,17-pentaoxo-5-oxa-2,7,10,13,16-pentaazaicosan-20-yl)oxy)-N,N3-di(2,5,8,11,14,17,20,23-octaoxapentacosan-25-yl)isophthalamide LD-90

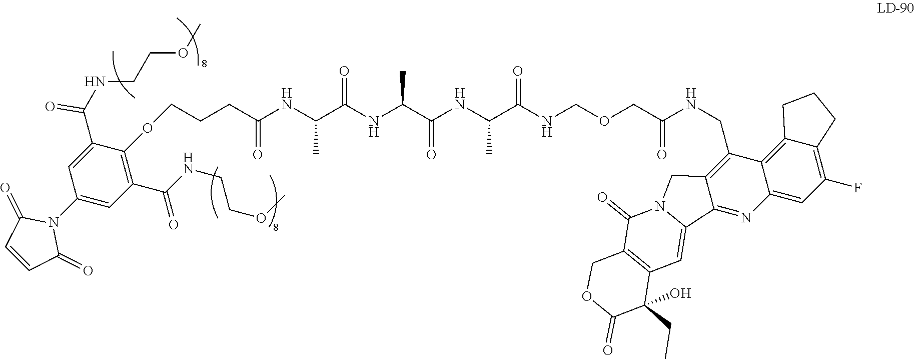

MS m/z (ESI): 907.0 [1/2(M+2)].

### Example 3-91: LD-91

### (S)-N5-((9S,12S,15S)-1-((S)-8-Ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-9,12-dimethyl-3,8,11,14-tetraoxo-5-oxa-2,7,10,13-tetraazahexadecan-15-yl)-2-(6-(2-(methylsulfonyl)pyrimidin-5-yl)he x-5-ynamido)-N¹-(2,5,8,11,14,17,20,23-octaoxapentacosan-25-yl)pentanediamide LD-91

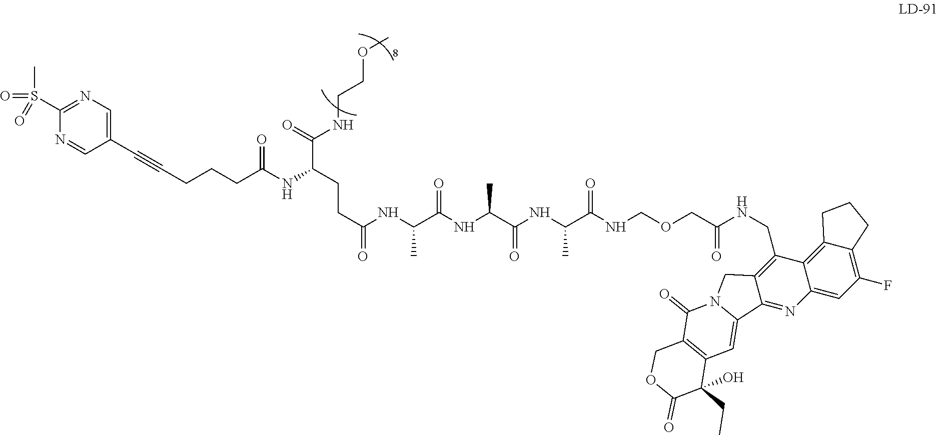

### Example 3-92: LD-92

### (S)-2-(6-(2,5-Dioxo-2,5-dihydro-1H-pyrrol-1-yl)hexanamido)-N⁵-((9S,12S,15S)-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-9,12-dimethyl-3,8,11,14-tetraoxo-5-oxa-2,7,10,13-tetraazahexadecan-15-yl)-N¹-(2,5,8,11,14,17,20,23-octaoxapentacosan-25-yl)pentanediamide LD-92

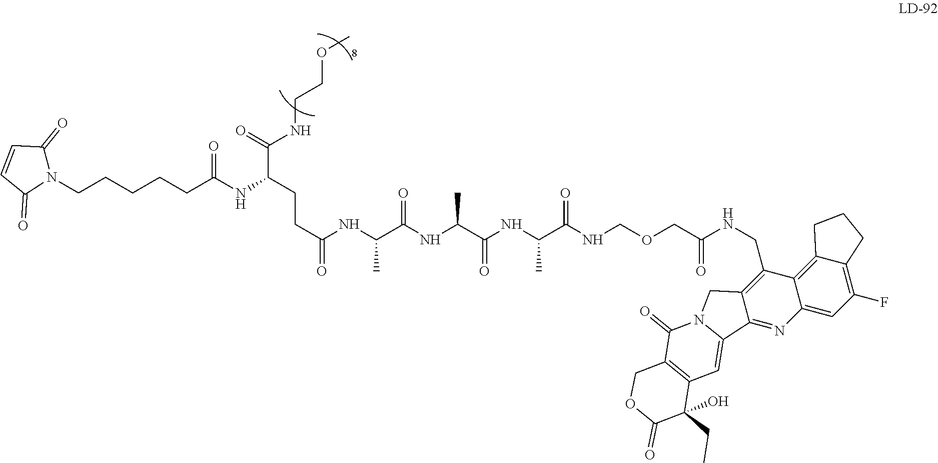

### Example 3-93: LD-93

### N-((9S,12S,15S)-1-((S)-4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-9,12-dimethyl-3,8,11,14-tetraoxo-5-oxa-2,7,10,13-tetraazahexadecan-15-yl)-6-(2,5-dioxo-2,5-dihydro-1H-pyrrol-1-yl)hexanamide LD-93

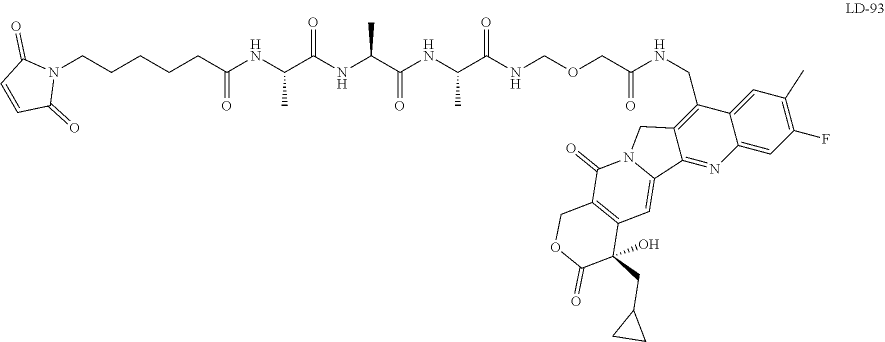 [SMILES: Cc1cc2c(CNC(=O)COCNC(=O)[C@H](C)NC(=O)[C@H](C)NC(=O)[C@H](C)NC(=O)CCCCCN3C(=O)C=CC3=O)c3c(nc2cc1F)-c1cc2c(c(=O)n1C3)COC(=O)[C@]2(O)CC1CC1]

### Example 3-94: LD-94

### 2-(((9S,12S,15S)-1-((S)-4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-3,8,11,14,17-pentaoxo-5-oxa-2,7,10,13,16-pentaazaicosan-20-yl)oxy)-5-(2,5-dioxo-2,5-dihydro-1H-pyrrol-1-yl)-N,N3-di(2,5,8,11,14,17,20,23-octaoxapentacosan-25-yl)isophthalamide LD-94

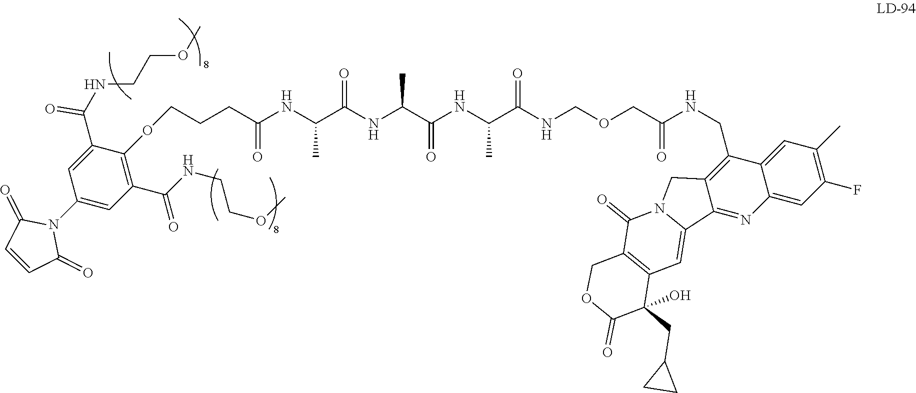

### Example 3-95: LD-95

### (S)-N5-((9S,12S,15S)-1-((S)-4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-9,12-dimethyl-3,8,11,14-tetraoxo-5-oxa-2,7,10,13-tetraazahexadecan-15-yl)-2-(6-(2-(methylsulfonyl)pyrimidin-5-yl) hex-5-ynamido)-N¹-(2,5,8,11,14,17,20,23-octaoxapentacosan-25-yl)pentanediamide LD-95

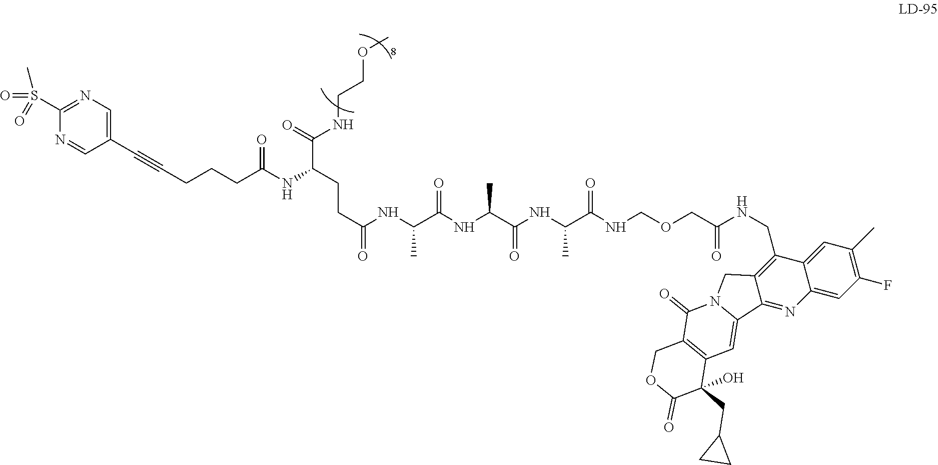

### Example 3-96: LD-96

### (S)-N5-((9S,12S,15S)-1-((S)-4-(Cyclopropylmethyl)-8-fluoro-4-hydroxy-9-methyl-3,14-dioxo-3,4,12,14-tetrahydro-1H-pyrano[3′,4′:6,7]indolizino[1,2-b]quinolin-11-yl)-9,12-dimethyl-3,8, 11,14-tetraoxo-5-oxa-2,7,10,13-tetraazahexadecan-15-yl)-2-(6-(2,5-dioxo-2,5-dihydro-1H-pyrrol-1-yl)hexanamido)-N¹-(2,5,8,11,14,17,20,23-octaoxapentacosan-25-yl)pentanediamide LD-96

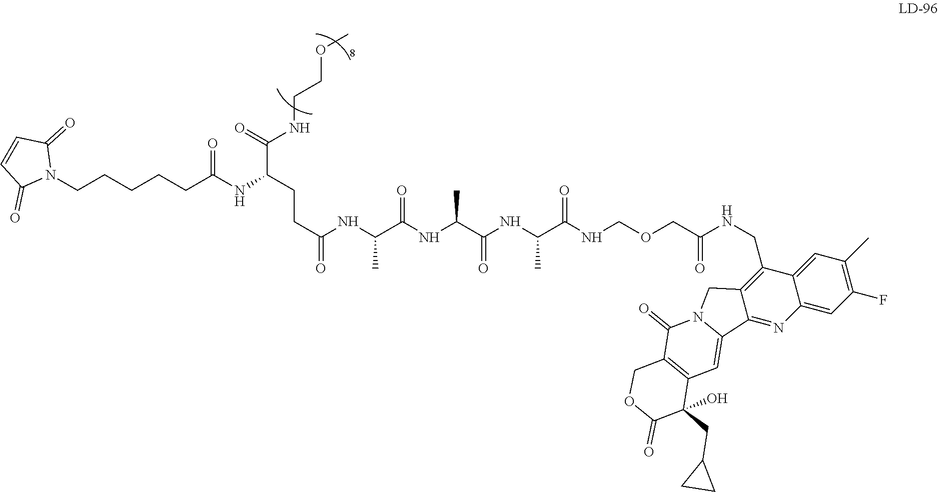

### Example 3-97: LD-97

### N-((S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-1-(4-(2-(methylsulfonyl)pyrimidin-5-yl)but-3-yn-1-yl)cyclopropane-1-carboxamide LD-97

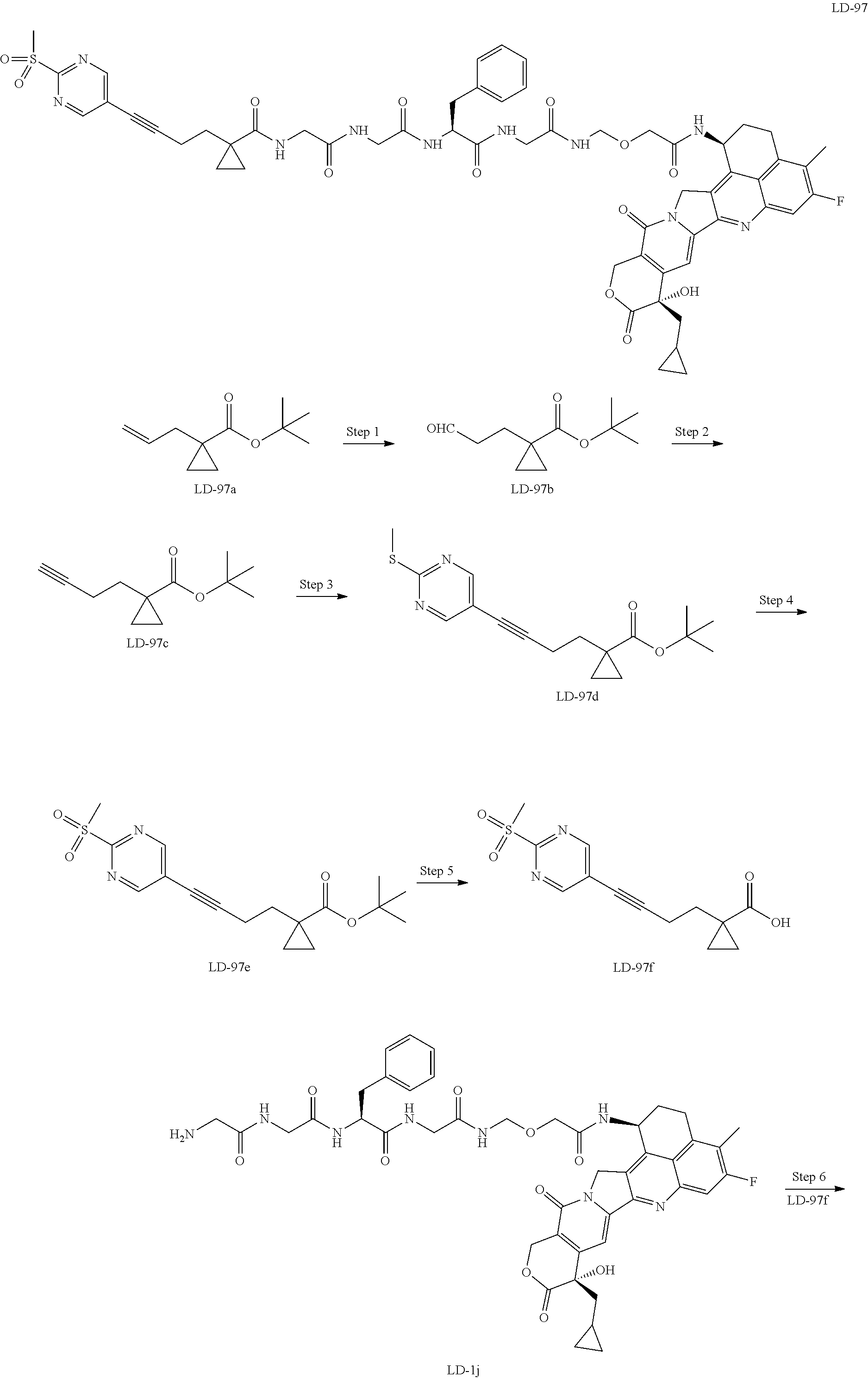

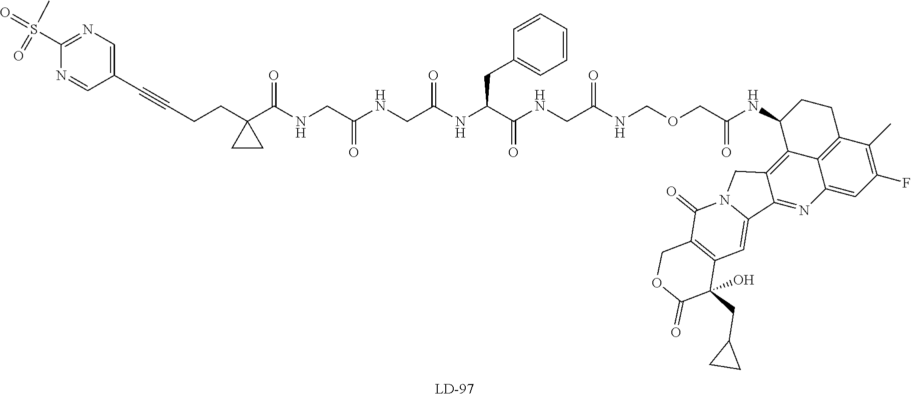 [SMILES: Cc1c(F)cc2nc3c(c4c2c1CC[C@@H]4NC(=O)COCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1]

### Step 1

LD-97a (2.20 g, 12.1 mmol, prepared by applying the method disclosed in Example 4 on page 62 of patent application “WO2020077038 A1”), silver nitrite (111 mg, 724 μmol), bis(benzonitrile)palladium(II) chloride (555 mg, 1.45 mmol), nitromethane (4.90 g, 80.3 mmol) and copper chloride dihydrate (II) (247 mg, 1.45 mmol) were dissolved in 44 mL of tert-butanol, subjected to replacement with oxygen gas three times, and stirred at 25° C. for 12 hours under oxygen gas. At 25° C., 150 mL of water was added to the reaction solution, dichloromethane (50 mL×3) was used for extraction, the organic phase was dried with anhydrous sodium sulfate and filtered, and the filtrate was concentrated by vacuum distillation. The residue was purified by silica gel column chromatography with the developing system B to obtain the title product LD-97b (800 mg, yield: 33%) as a white solid.

### Step 2

LD-97b (800 mg, 4.04 mmol) was dissolved in 24 mL of methanol, then added with potassium carbonate (1.12 g, 8.07 mmol) and dimethyl (1-diazo-2-oxopropyl)phosphonate (930 mg, 4.84 mmol), and stirred at 25° C. for 12 hours. 50 mL of water was added to the reaction solution, and ethyl acetate (20 mL×3) was used for extraction. The organic phase was dried over anhydrous sodium sulfate and filtered, and the filtrate was concentrated by vacuum distillation. The residue was purified by silica gel column chromatography with the developing system B to obtain the title product LD-97c (650 mg, yield: 83%) as a white solid.

### Step 3

LD-97c (650 mg, 3.35 mmol), 5-bromo-2-methylthiopyrimidine (686 mg, 3.35 mmol), triethylamine (3.39 g, 33.4 mmol), cuprous iodide (63.7 mg, 334 μmol) and bistriphenylphosphine palladium dichloride (244 mg, 349 μmol) were dissolved in 10 mL of tetrahydrofuran, subjected to replacement with nitrogen gas three times, and stirred at 60° C. for 2 hours under nitrogen protection. The reaction solution was concentrated under reduced pressure, and the obtained residue was purified by silica gel column chromatography with the developing system B to obtain the title product LD-97d (770 mg, yield: 68%) as a white solid.

MS m/z (ESI): 319.6 [M+1].

### Step 4

LD-97d (1.80 g, 5.65 mmol) was dissolved in 20 mL of dichloromethane, then added with m-chloroperbenzoic acid (2.87 g, 14.1 mmol, purity 85.0%), and stirred at 25° C. for 2 hours. The reaction solution was concentrated under reduced pressure, and the obtained residue was purified by silica gel column chromatography with the developing system B to obtain the title product LD-97e (1.70 g, yield: 85%) as a white solid.

MS m/z (ESI): 351.1 [M+1].

### Step 5

LD-97e (500 mg, 1.43 mmol) was dissolved in 6 mL of dichloromethane, then added with trifluoroacetic acid (2.30 g, 20.2 mmol), and stirred at 25° C. for 2 hours. The reaction solution was concentrated under reduced pressure to obtain the crude title product LD-97f (418 mg) as a white solid. The product was used directly in the next step without purification.

MS m/z (ESI): 295.2 [M+1].

### Step 6

LD-97f (14.2 mg, 48.4 μmol) and LD-1j (35.0 mg, 40.3 μmol) were dissolved in 2 mL of N,N-dimethylformamide, then added with 1-hydroxybenzotriazole (8.2 mg, 60.5 μmol), N,N-diisopropylethylamine (15.6 mg, 121 μmol) and 1-(3-dimethylaminopropyl)-3-ethylcarbodiimide hydrochloride (11.6 mg, 60.6 μmol) in sequence, and stirred at 25° C. for 2 hours. The reaction solution was concentrated under reduced pressure to remove the solvent, 1 mL of water was added to the residue, dichloromethane (3 mL×2) was used for extraction, the organic phase was washed with saturated sodium chloride solution (2 mL), then dried over anhydrous sodium sulfate and filtered, and the filtrate was concentrated under reduced pressure. The residue was purified by pre-HPLC (chromatographic column: Phenomenex Kinetex EVO C18 150×30 mm×5 μm; mobile phase: A-water (0.225% formic acid), B-acetonitrile; gradient elution: B %: 35%-55%) to obtain the title product LD-97 (8.00 mg, yield: 17%) as a yellow solid.

MS m/z (ESI): 1143.5 [M+1].

¹H NMR (400 MHz, DMSO-d6) δ 9.11-9.00 (m, 2H), 8.67-8.59 (m, 1H), 8.55-8.48 (m, 1H), 8.34-8.28 (m, 1H), 8.17-8.11 (m, 1H), 8.00-7.94 (m, 1H), 7.90-7.83 (m, 1H), 7.81-7.75 (m, 1H), 7.41-7.35 (m, 1H), 7.28-7.11 (m, 5H), 6.60-6.55 (m, 1H), 5.63-5.56 (m, 1H), 5.47-5.35 (m, 2H), 5.25-5.17 (m, 2H), 4.70-4.59 (m, 2H), 4.51-4.43 (m, 1H), 4.07-3.98 (m, 2H), 3.79-3.53 (m, 6H), 3.44-3.36 (m, 3H), 3.20-3.13 (m, 2H), 3.05-2.99 (m, 1H), 2.81-2.73 (m, 1H), 2.71-2.62 (m, 2H), 2.40-2.36 (m, 3H), 2.22-2.13 (m, 2H), 1.92-1.85 (m, 2H), 1.85-1.79 (m, 1H), 1.78-1.71 (m, 1H), 1.04-0.95 (m, 2H), 0.83-0.76 (m, 1H), 0.73-0.65 (m, 2H), 0.37-0.25 (m, 2H), 0.09-0.01 (m, 1H), −0.05-−0.12 (m, 1H).

### Example 3-98: LD-98

### N-((2S,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoli n-1-yl)amino)-2-methyl-1,6,9,12,15-pentaoxo-3-oxa-5,8,11,14-tetraazahexadecan-16-yl)-1-(4-(2-(methylsulfonyl)pyrimidin-5-yl)but-3-yn-1-yl)cyclopropane-1-carboxamide LD-98

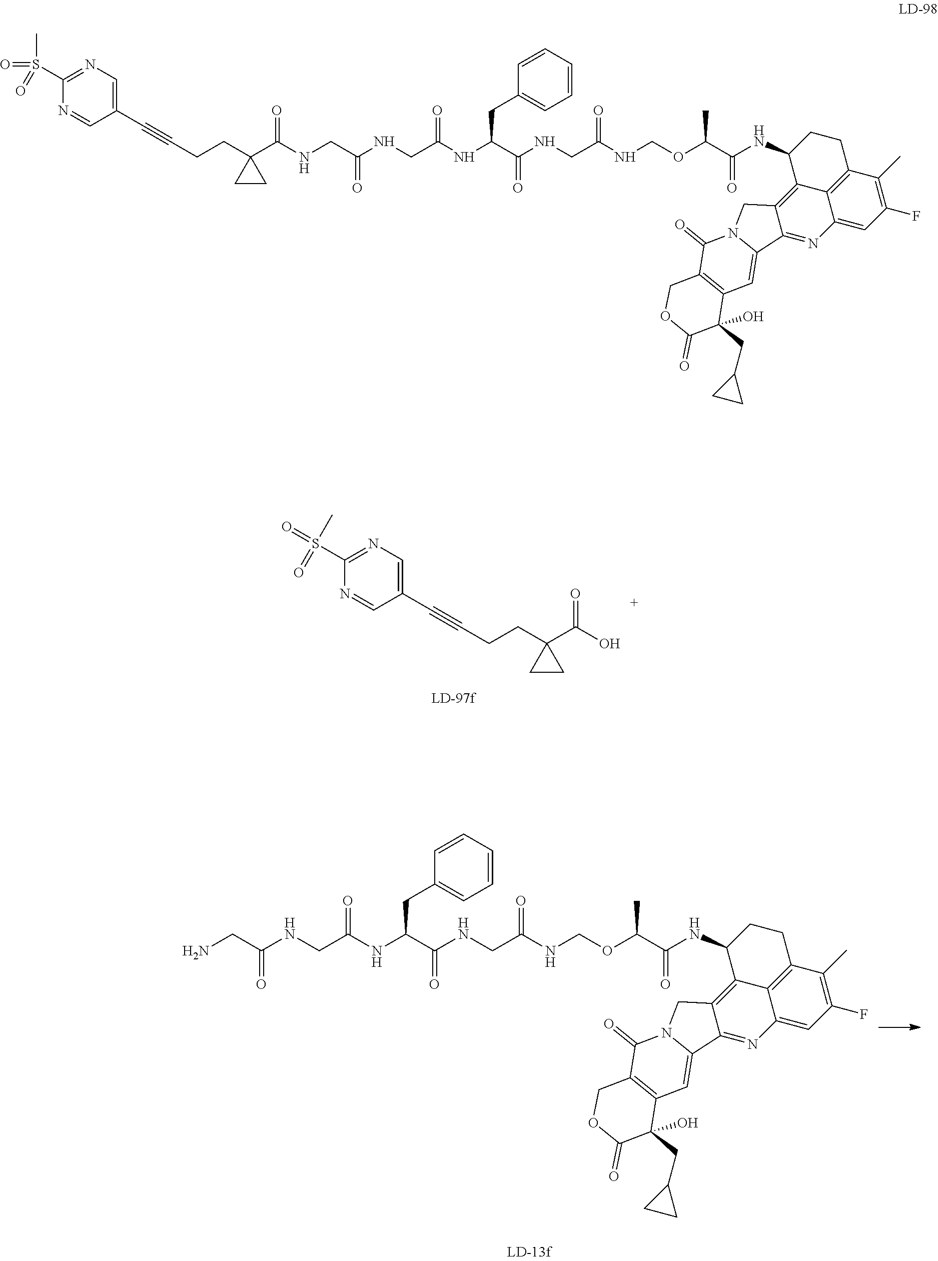 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1 ; CS(=O)(=O)c1ncc(C#CCCC2(C(=O)O)CC2)cn1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CN)Cn1c-3cc2c(c1=O)COC(=O)[C@]2(O)CC1CC1]

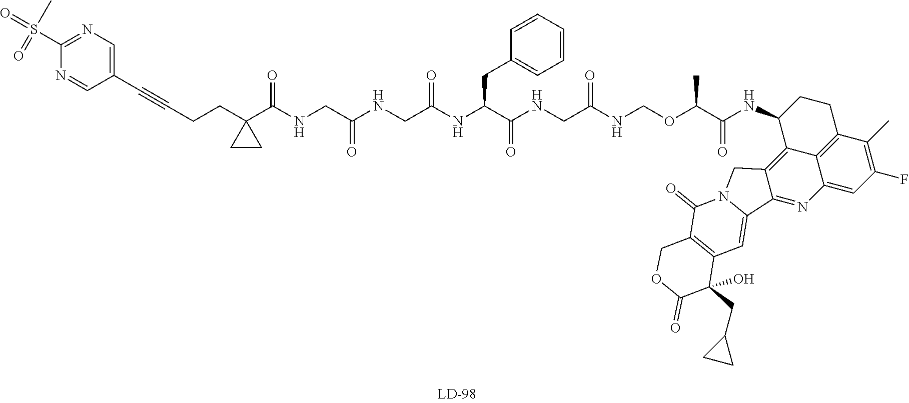 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1]

LD-97f (10.8 mg, 36.8 μmol) and LD-13f (27.0 mg, 30.6 μmol) were dissolved in 1 mL of N,N-dimethylformamide, added with 1-hydroxybenzotriazole (6.21 mg, 45.9 μmol), N,N-diisopropylethylamine (11.9 mg, 91.9 μmol) and 1-(3-dimethylaminopropyl)-3-ethylcarbodiimide hydrochloride (8.81 mg, 45.9 μmol), and stirred at 25° C. 1 hour. The reaction solution was concentrated under reduced pressure to remove the solvent, 3 mL of water was added to the residue, and dichloromethane (3 mL×3) was used for extraction. The organic phase was washed with saturated sodium chloride solution (2 mL), and then dried over anhydrous sodium sulfate, and filtered. The filtrate was concentrated under reduced pressure. The obtained residue was purified by silica gel column chromatography using the eluent system A to obtain a crude product, and the crude product was then purified by pre-HPLC (chromatographic column: Phenomenex C18 150 mm×30 mm×5 μm; mobile phase: A -water (0.1% trifluoroacetic acid), B-acetonitrile; gradient elution: B %: 25%-55%) to obtain the title product LD-98 (2.11 mg, yield: 6%) as a white solid.

MS m/z (ESI): 1157.8 [M+1].

¹H NMR (400 MHz, CD₃OD) δ 8.89-8.80 (m, 2H), 7.66-7.58 (m, 2H), 7.26-7.19 (m, 2H), 7.19-7.08 (m, 3H), 5.71-5.65 (m, 1H), 5.62-5.54 (m, 1H), 5.50-5.42 (m, 1H), 5.33-5.25 (m, 1H), 5.22-5.15 (m, 1H), 4.78-4.71 (m, 2H), 4.33-4.25 (m, 2H), 3.91-3.72 (m, 4H), 3.61-3.45 (m, 3H), 3.36-3.33 (m, 3H), 3.22-3.12 (m, 1H), 2.94-2.84 (m, 2H), 2.73-2.64 (m, 2H), 2.46-2.37 (m, 3H), 2.37-2.28 (m, 1H), 2.26-2.18 (m, 1H), 2.01-1.90 (m, 2H), 1.89-1.81 (m, 1H), 1.76-1.69 (m, 1H), 1.51-1.42 (m, 3H), 1.17-1.12 (m, 2H), 0.94-0.83 (m, 3H), 0.45-0.31 (m, 2H), 0.06-−0.08 (m, 2H).

### Example 3-99: LD-99

### N-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-1-(4-(2-(methylsulfonyl)pyrimidin-5-yl)but -3-yn-1-yl)cyclopropane-1-carboxamide LD-99

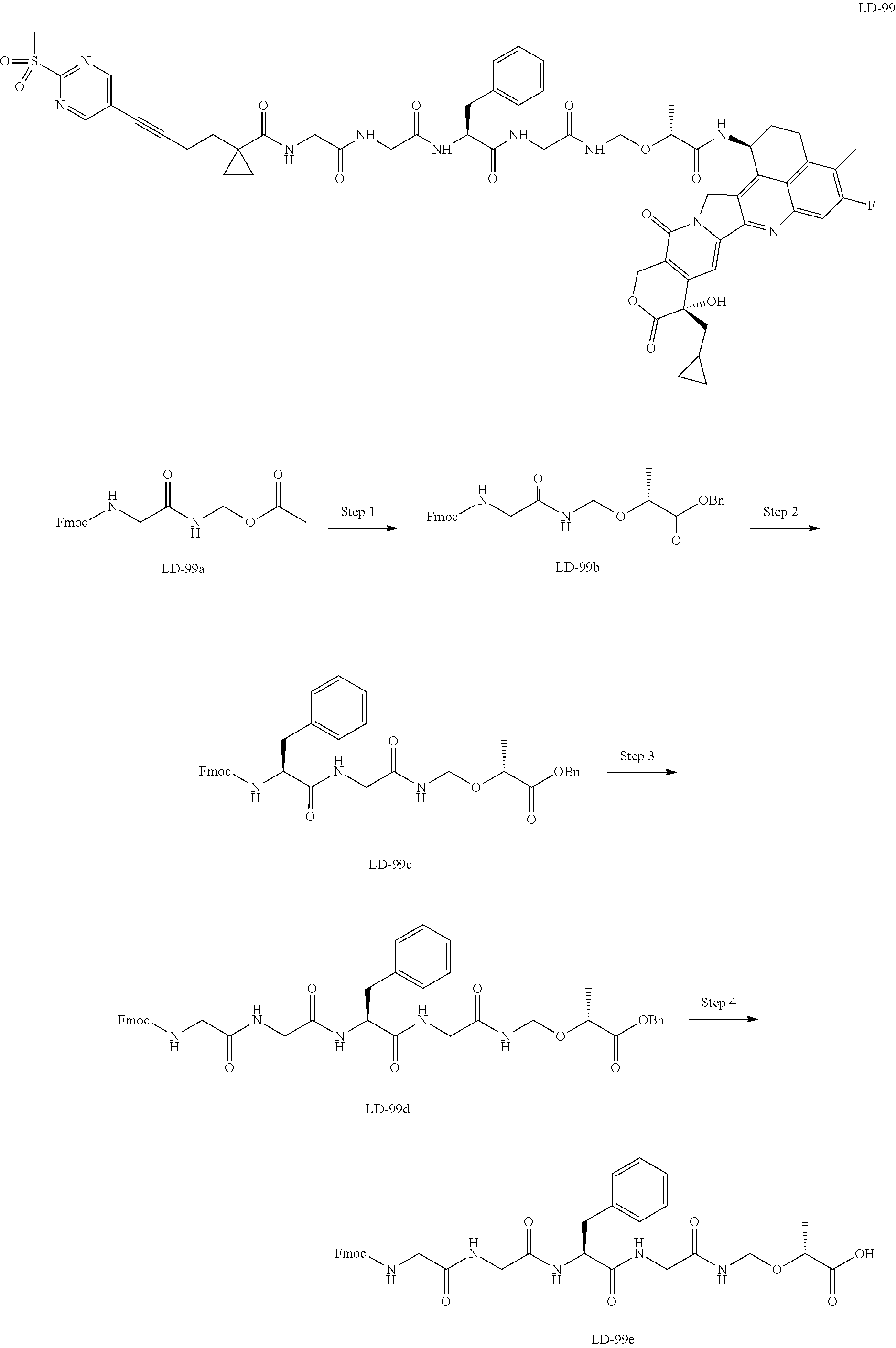

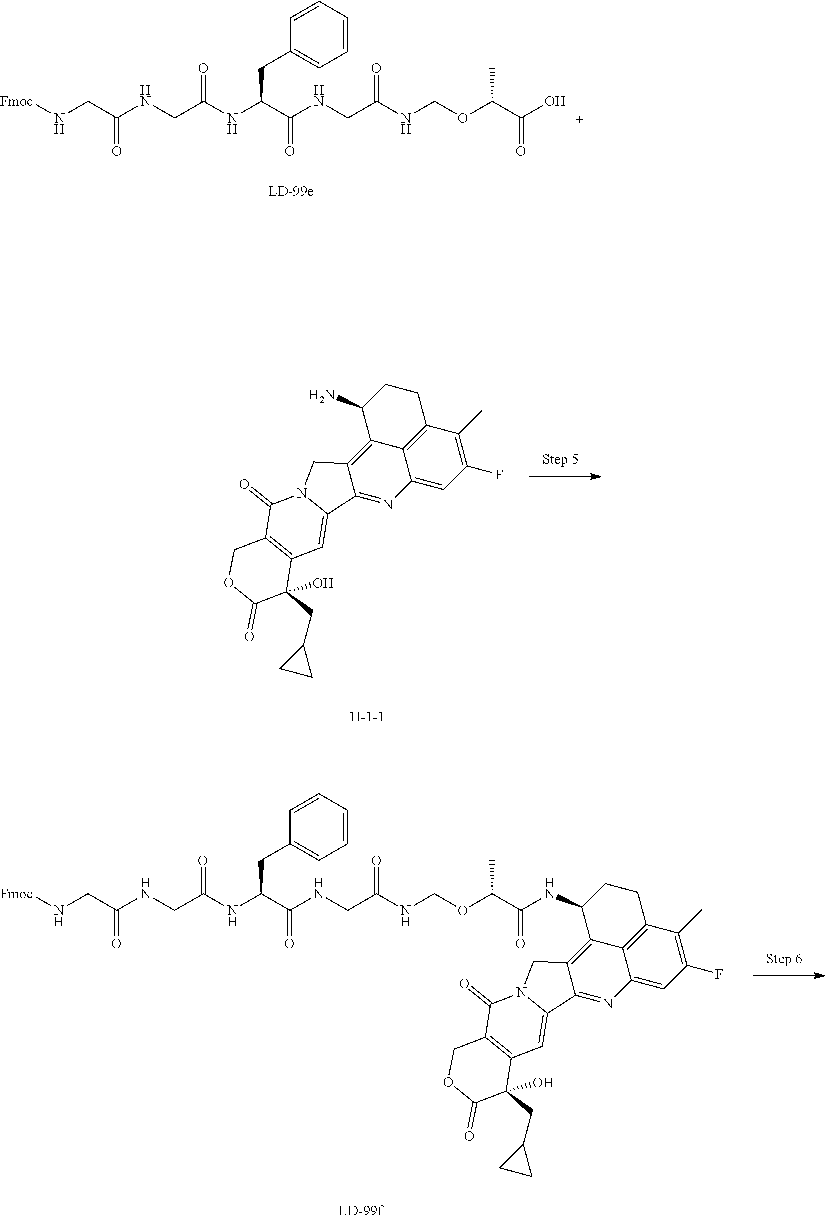

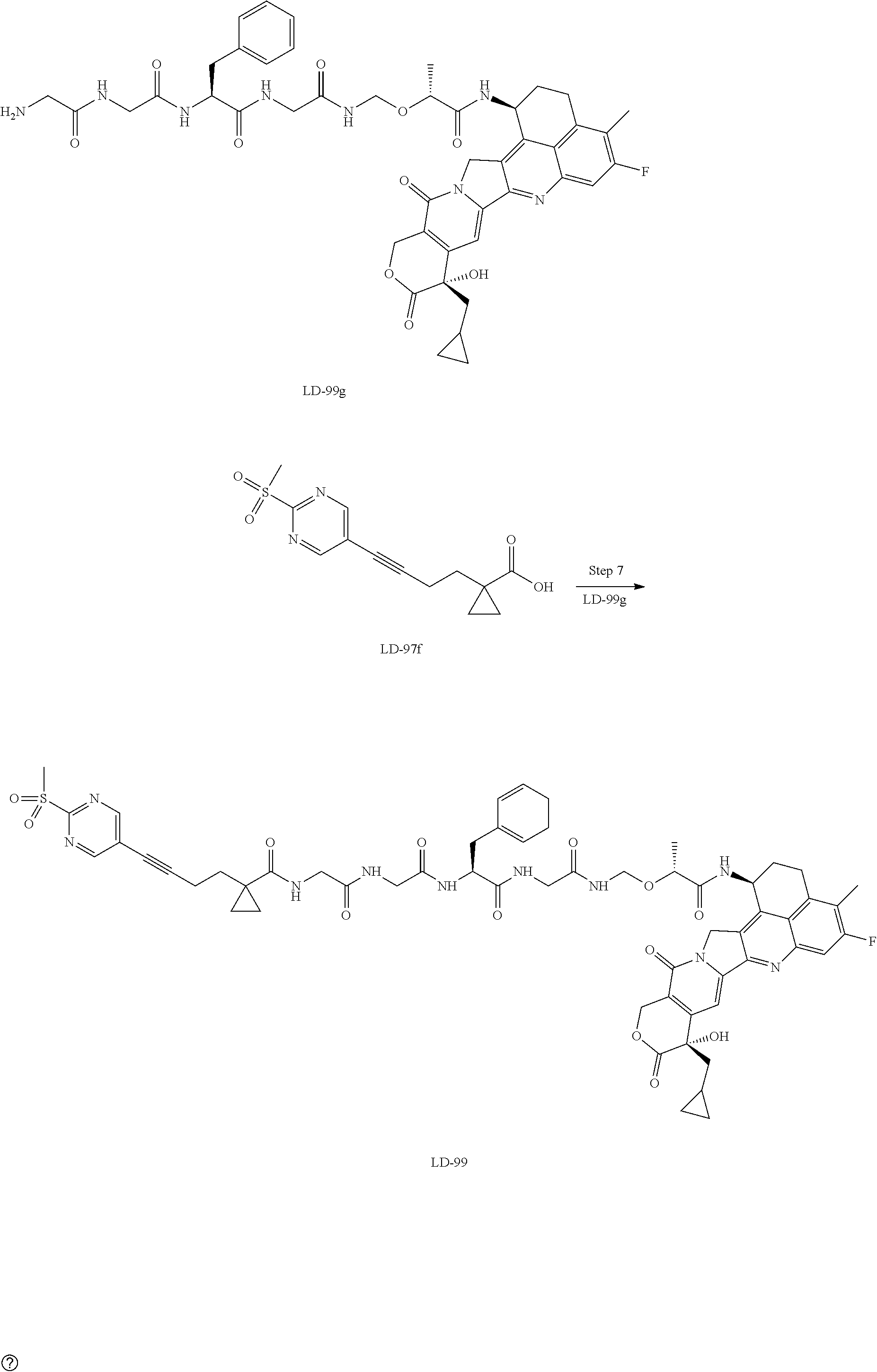 [SMILES: Cc1c(F)cc2nc3c(c4c2c1CC[C@@H]4NC(=O)[C@@H](C)OCNC(=O)CNC(=O)[C@H](Cc1ccccc1)NC(=O)CNC(=O)CN)Cn1c-3cc2c(c1=O)COC(=O)[C@]2(O)CC1CC1 ; CS(=O)(=O)c1ncc(C#CCCC2(C(=O)O)CC2)cn1 ; Cc1c(F)cc2nc3c(c4c2c1CC[C@@H]4NC(=O)[C@@H](C)OCNC(=O)CNC(=O)[C@H](CC1=CCCC=C1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1]

### Step 1

### Benzyl(R)-1-(9H-fluorene-9-yl)-10-methyl-3,6-dioxo-2,9-dioxa-4,7-diazaundecan-11-oate LD-99b

LD-99a (3.00 g, 7.87 mmol, prepared by applying the method disclosed in Example 1.15 on page 226 of patent application “WO2022135332 A1”) was dissolved in 50 mL of dichloromethane, added with pyridium 4-toluenesulfonate (1.98 g, 7.87 mmol) and benzyl (2R)-2-hydroxypropanoate (2.84 g, 15.7 mmol), and stirred at 50° C. for 10 hours. 40 mL of water was added to the reaction solution, and dichloromethane (50 mL×3) was used for extraction. The organic phase was dried over anhydrous sodium sulfate and filtered, and the filtrate was concentrated by distillation under reduced pressure. The resulting residue was purified by silica gel column chromatography using the developing system B to obtain the title product LD-99b (1.55 g, yield:: 40%).

### Step 2

### Benzyl(5S,13R)-5-benzyl-1-(9H-fluoren-9-yl)-13-methyl-3,6,9-trioxo-2,12-dioxa-4,7,10-triazatetradecan-14-oate LD-99c

LD-99b (2.63 g, 5.38 mmol) was dissolved in 30 mL of dichloromethane, added with 1,8-diazabicyclo[5.4.0]undec-7-ene (491 mg, 3.23 mmol), and stirred at 25° C. for 1 hour. Then, (((9H-fluoren-9-yl)methoxy)carbonyl)-L-phenylalanine (2.29 g, 5.91 mmol), 1-hydroxybenzotriazole (1.09 g, 8.06 mmol) and 1-(3-dimethylaminopropyl)-3-ethylcarbodiimide hydrochloride (1.54 g, 8.06 mmol) added to the reaction solution, and stirred at 25° C. for 2 hours. 10 mL of water was added, and dichloromethane (10 mL×3) was used for extraction, the organic phase was dried over anhydrous sodium sulfate and filtered, and the filtrate was concentrated by vacuum distillation.

The obtained residue was purified by silica gel column chromatography using the eluent system A to obtain the title product LD-99c (1.60 g, yield: 37%) as a yellow solid.

MS m/z (ESI): 658.4 [M+23].

Step 3

### Benzyl(11S,19R)-11-benzyl-1-(9H-fluoren-9-yl)-19-methyl-3,6,9,12,15-pentaoxo-2,18-dioxa-4,7,10,13,16-pentaazaicosan-20-oate LD-99d

LD-99c (1.60 g, 1.97 mmol) was dissolved in 15 mL of dichloromethane, added with 1,8-diazabicyclo[5.4.0]undec-7-ene (179 mg, 1.18 mmol), and stirred at 25° C. for 1 hour. Then, (((9H-fluoren-9-yl)methoxy)carbonyl)glycylglycine (836 mg, 2.36 mmol) and O-(7-azabenzotriazol-1-yl)-N,N,N′,N′-tetramethyluronium hexafluorophosphate (1.12 g, 2.95 mmol) were added in sequence to the reaction solution, and stirred at 25° C. for 1 hour. 10 mL of water was added, dichloromethane (10 mL×3) was used for extraction, and the organic phase was dried over anhydrous sodium sulfate and filtered, and the filtrate was concentrated by distillation under reduced pressure. The resulting residue was purified by silica gel column chromatography using the eluent system A to obtain the title product LD-99d (1.15 g, yield: 70%) as a yellow solid.).

MS m/z (ESI): 772.4 [M+23].

### Step 4

### (11S,19R)-11-Benzyl-1-(9H-fluoren-9-yl)-19-methyl-3,6,9,12,15-pentaoxo-2,18-dioxa-4,7,10,13,16-pentaazaicosan-20-oic acid LD-99e

Under nitrogen protection, LD-99d (500 mg, 602 μmol) and palladium-on-carbon (448 mg, 10%, weitted with ca. 55% water) was dissolved in 2 mL of ethyl acetate and 4 mL of ethanol, and subjected to replacement with hydrogen gas three times. The reaction solution was stirred at 25° C. for 2 hours under hydrogen gas (15 Psi). The reaction solution was filtered through Celite, and the filtrate was concentrated by distillation under reduced pressure to obtain the crude title product LD-99e (330 mg, yield: 76%) as a white solid. The product was used directly in the next reaction without purification.

MS m/z (ESI): 682.4 [M+23].

### Step 5

### (9H-Fluoren-9-yl)methyl((2R,10S)-10-benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)carbamate LD-99f

LD-99e (30.2 mg, 41.7 μmol) and 1i-1-1 (15.0 mg, 32.1 μmol) were dissolved in 2 mL of N,N-dimethylformamide, added with O-(7-azabenzotriazol-1-yl)-N,N,N′,N′-tetramethyluronium hexafluorophosphate (18.3 mg, 48.1 μmol) and N,N-diisopropylethylamine (12.4 mg, 96.2 μmol) in sequence, and stirred at 25° C. for 1 hour. 1 mL of water was added, dichloromethane (3 mL×2) was used for extraction. The organic phase was dried over anhydrous sodium sulfate and filtered, and the filtrate was concentrated by vacuum distillation. The obtained residue was purified by silica gel column chromatography with the eluent system A to obtain the title product LD-99f (25.0 mg, yield: 59%) as a yellow solid.

MS m/z (ESI): 1103.6 [M+1].

### Step 6

### (S)-2-(2-(2-Aminoacetamido)acetamido)-N-(2-(((((R)-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)oxy)methyl)amino)-2-oxyethyl)-3-phenylpropanamide LD-99 g

LD-99f (44.0 mg, 39.9 μmol) was dissolved in 1 mL of acetonitrile, added with diethylamine (142 mg, 1.94 mmol), and stirred at 25° C. for 0.5 h. The reaction solution was concentrated by vacuum distillation to obtain the crude title product LD-99 g (30.0 mg) as a yellow solid. The product was used directly in the next reaction without purification.

MS m/z (ESI): 881.5 [M+1].

### Step 7

### N-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-1-(4-(2-(methylsulfonyl)pyrimidin-5-yl)but-3-yn-1-yl)cyclopropane-1-carboxamide LD-99

LD-97f (16.0 mg, 54.5 μmol) and LD-99 g (40.0 mg, 45.4 μmol) were dissolved in 1 mL of N,N-dimethylformamide, then added with 1-hydroxybenzotriazole (9.20 mg, 68.1 μmol), N,N-diisopropylethylamine (23.5 mg, 181 μmol) and 1-(3-dimethylaminopropyl)-3-ethylcarbodiimide hydrochloride (13.1 mg, 68.1 μmol) in sequence, and stirred at 25° C. for 1 hour. The reaction solution was concentrated under reduced pressure to remove the solvent, and the resulting residue was purified by pre-HPLC (chromatographic column: Welch Xtimate C18 150×25 mm×5 μm; mobile phase: A-water (0.1% trifluoroacetic acid), B-acetonitrile; gradient elution: B %: 36%-56%) to obtain the title product LD-99 (10.6 mg, yield: 69%) as a white solid.

MS m/z (ESI): 1157.2 [M+1].

¹H NMR (400 MHz, DMSO-d6) δ 9.12-9.00 (m, 2H), 8.68-8.60 (m, 1H), 8.60-8.52 (m, 1H), 8.32-8.26 (m, 1H), 8.15-8.08 (m, 1H), 8.01-7.92 (m, 1H), 7.90-7.84 (m, 1H), 7.81-7.74 (m, 1H), 7.36 (s, 1H), 7.27-7.10 (m, 5H), 6.61-6.56 (m, 1H), 5.63-5.56 (m, 1H), 5.46-5.35 (m, 2H), 5.25-5.07 (m, 2H), 4.74-4.66 (m, 1H), 4.58-4.50 (m, 1H), 4.50-4.43 (m, 1H), 4.17-4.08 (m, 1H), 3.79-3.53 (m, 6H), 3.44-3.37 (m, 3H), 3.18-3.05 (m, 2H), 3.04-2.96 (m, 1H), 2.78-2.63 (m, 3H), 2.39-2.35 (m, 3H), 2.23-2.13 (m, 2H), 1.94-1.84 (m, 2H), 1.83-1.71 (m, 2H), 1.48-1.36 (m, 3H), 1.06-0.95 (m, 2H), 0.86-0.77 (m, 1H), 0.75-0.66 (m, 2H), 0.39-0.24 (m, 2H), 0.09-0.01 (m, 1H), −0.04-−0.13 (m, 1H).

### Example 3-100: LD-100

### N-((S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-3,8,11,14,17-pentaoxo-5-oxa-2,7,10,13,16-pentaazaoctadecan-18-yl)-1-(4-(2-(methylsulfonyl)pyrimidin-5-yl)but-3-yn-1-yl)cyclopropane-1-carboxamide LD-100

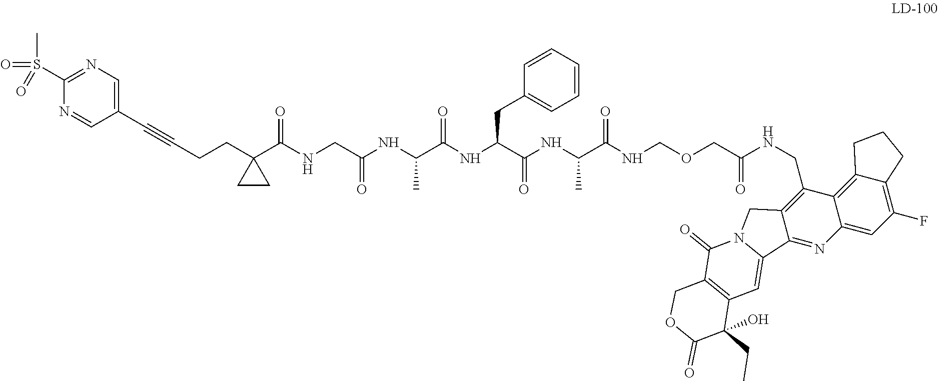 [SMILES: CC[C@@]1(O)C(=O)OCc2c1cc1n(c2=O)Cc2c-1nc1cc(F)c3c(c1c2CNC(=O)COCNC(=O)[C@H](C)NC(=O)[C@H](Cc1ccccc1)NC(=O)[C@H](C)NC(=O)CNC(=O)C1(CCC#Cc2cnc(S(C)(=O)=O)nc2)CC1)CCC3]

### Example 3-101: LD-101

### N-((4S,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17-pentaoxo-5-oxa-2,7,10,13,16-pentaazaoctadecan-18-yl)-1-(4-(2-(methylsulfonyl)pyrimidin-5-yl)but-3-yn-1-yl)cyclopropane-1-carboxamide LD-101

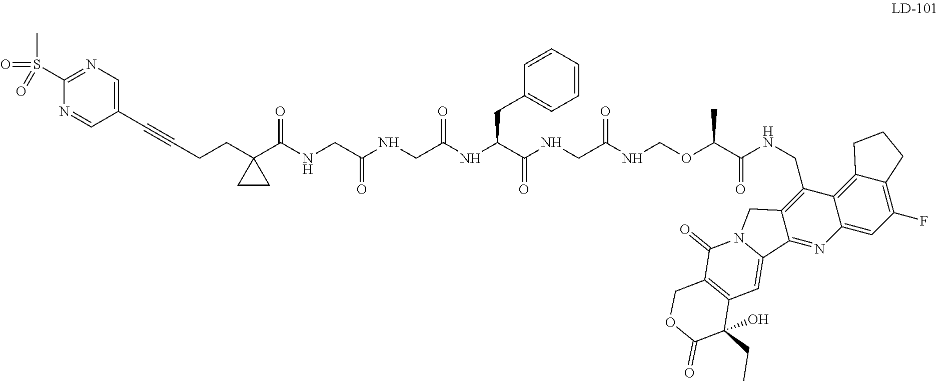 [SMILES: CC[C@@]1(O)C(=O)OCc2c1cc1n(c2=O)Cc2c-1nc1cc(F)c3c(c1c2CNC(=O)[C@H](C)OCNC(=O)CNC(=O)[C@H](Cc1ccccc1)NC(=O)CNC(=O)CNC(=O)C1(CCC#Cc2cnc(S(C)(=O)=O)nc2)CC1)CCC3]

### Example 3-102: LD-102

### N-((4R,12S)-12-Benzyl-1-((S)-8-ethyl-4-fluoro-8-hydroxy-9,12-dioxo-2,3,8,9,12,14-hexahydro-1H,11H-cyclopenta[f]pyrano[3′,4′:6,7]indolizino[1,2-b]quinolin-15-yl)-4-methyl-3,8,11,14,17-pentaoxo-5-oxa-2,7,10,13,16-pentaazaoctadecan-18-yl)-1-(4-(2-(methylsulfonyl)pyrimidin-5-yl)but-3-yn-1-yl)cyclopropane-1-carboxamide LD-102

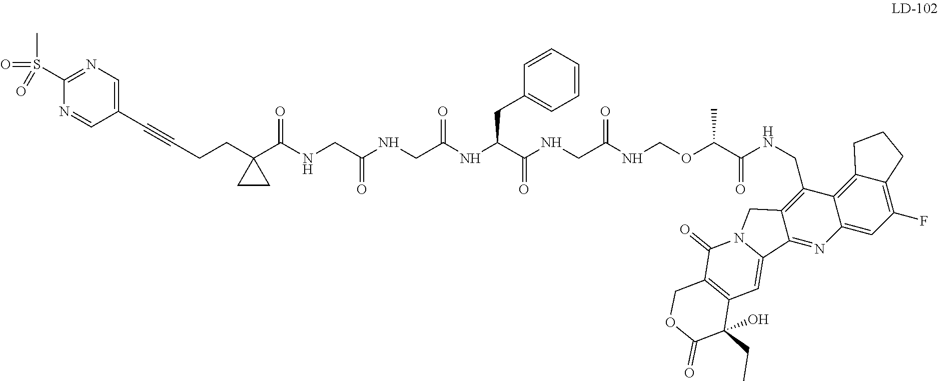 [SMILES: CC[C@@]1(O)C(=O)OCc2c1cc1n(c2=O)Cc2c-1nc1cc(F)c3c(c1c2CNC(=O)[C@@H](C)OCNC(=O)CNC(=O)[C@H](Cc1ccccc1)NC(=O)CNC(=O)CNC(=O)C1(CCC#Cc2cnc(S(C)(=O)=O)nc2)CC1)CCC3]

### Example 3-103: LD-103

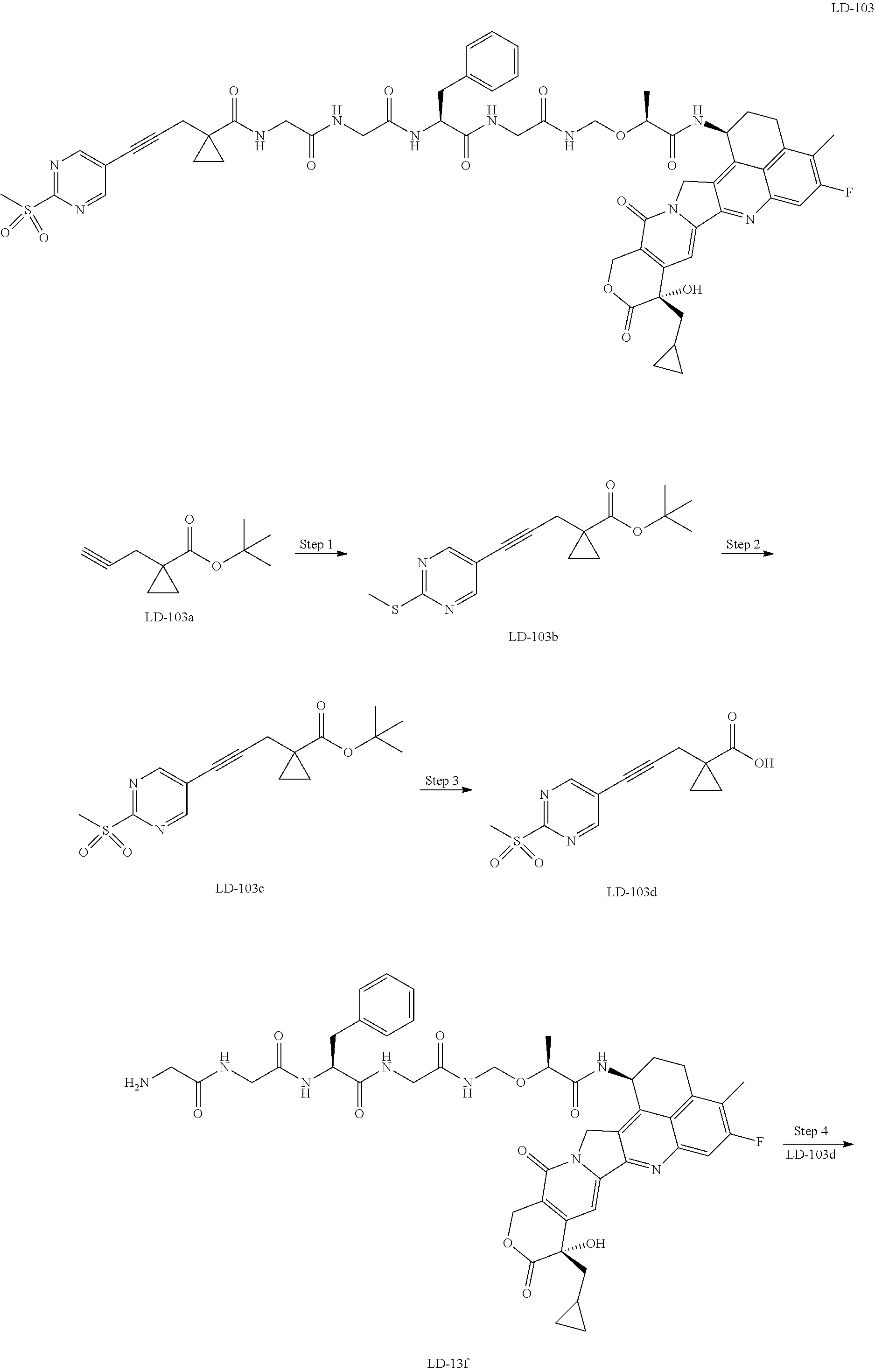 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CC#Cc2cnc(S(C)(=O)=O)nc2)CC1)Cn1c-3cc2c(c1=O)COC(=O)[C@]2(O)CC1CC1 ; C#CCC1(C(=O)OC(C)(C)C)CC1 ; CSc1ncc(C#CCC2(C(=O)OC(C)(C)C)CC2)cn1 ; CC(C)(C)OC(=O)C1(CC#Cc2cnc(S(C)(=O)=O)nc2)CC1 ; CS(=O)(=O)c1ncc(C#CCC2(C(=O)O)CC2)cn1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CN)Cn1c-3cc2c(c1=O)COC(=O)[C@]2(O)CC1CC1]

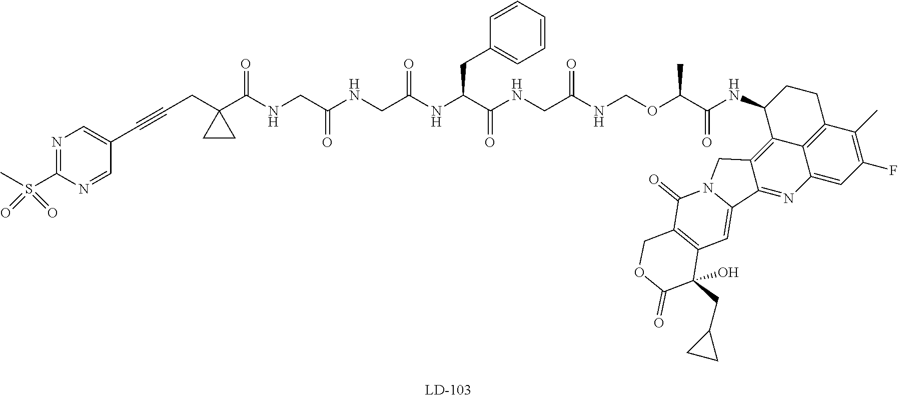 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CC#Cc2cnc(S(C)(=O)=O)nc2)CC1)Cn1c-3cc2c(c1=O)COC(=O)[C@]2(O)CC1CC1]

### Step 1

LD-103a (1.00 g, 5.55 mmol, prepared by applying the method disclosed in Example 34 on page 124 of patent application “WO2022135332 A1”), 5-bromo-2-methylthiopyrimidine (1.14 g, 5.55 mmol), triethylamine (2.42 g, 23.9 mmol), bistriphenylphosphine palladium dichloride (389 mg, 554 μmol) and cuprous iodide (105 mg, 554 μmol) were dissolved in 6 mL of tetrahydrofuran, subjected to replacement with nitrogen gas three times, and stirred at 60° C. for 2 hours under nitrogen protection. The reaction solution was filtered through Celite, 20 mL of water was added to the filtrate, ethyl acetate (15 mL×3) was used for extraction, the organic phase was dried over anhydrous sodium sulfate and filtered, and the filtrate was concentrated by vacuum distillation. The resulting residue was purified by silica gel column chromatography with the developing system B to obtain the title product LD-103b (502 mg, yield: 30%) as a yellow solid.

MS m/z (ESI): 305.5 [M+1].

### Step 2

LD-103b (450 mg, 1.43 mmol) was dissolved in 5 mL of dichloromethane, cooled to 0° C., slowly added with m-chloroperbenzoic acid (640 mg, 3.15 mmol, purity 85%), and stirred at 25° C. for 2 hours. Sodium thiosulfate aqueous solution (50 mL) was added, dichloromethane (45 mL×3) was used for extraction, the organic phase was dried over anhydrous sodium sulfate and filtered, and the filtrate was concentrated by vacuum distillation. The residue was purified by silica gel column chromatography with the developing system B to obtain the title product LD-103c (250 mg, yield: 50%) as a yellow solid.

MS m/z (ESI): 337.1 [M+1].

### Step 3

LD-103c (100 mg, 273 μmol) was dissolved in 1 mL of dichloromethane, added with trifluoroacetic acid (423 mg, 3.72 mmol), and stirred at 25° C. for 2 hours. The reaction solution was concentrated by vacuum distillation to obtain the crude title product LD-103d (80 mg) as a yellow solid. The product was used directly in the next step without purification.

MS m/z (ESI): 280.9 [M+1].

### Step 4

LD-13f (19.0 mg, 21.6 μmol) and LD-103d (7.23 mg, 23.7 μmol) were dissolved in 1 mL of N,N-dimethylformamide, then added 1-hydroxybenzotriazole (4.37 mg, 32.4 μmol), N,N-diisopropylethylamine (8.36 mg, 64.7 μmol) and 1-(3-dimethylaminopropyl)-3-ethylcarbodiimide hydrochloride (6.20 mg, 32.4 μmol) in sequence, and stirred at 20° C. for 2 hours. 50 mL of water was added to the reaction solution, and extracted with dichloromethane (45 mL×3). The organic phase was dried over anhydrous sodium sulfate and filtered, and the filtrate was concentrated by vacuum distillation. The resulting residue was purified by pre-HPLC (chromatographic column: Welch Xtimate C18 150×25 mm×5 μm; mobile phase: A-water (0.1% trifluoroacetic acid), B-acetonitrile; gradient elution: B %: 28%-58%), and freeze-dried to obtain a crude product, and the crude product was further purified by silica gel column chromatography with the eluent system A to obtain the title product LD-103 (1.50 mg, yield: 6%) as a white solid.

MS m/z (ESI): 1143.2 [M+1].

¹H NMR (400 MHz, CD₃OD) δ 8.94-8.88 (m, 2H), 7.67-7.61 (m, 2H), 7.26-7.20 (m, 2H), 7.19-7.10 (m, 3H), 5.71-5.66 (m, 1H), 5.60-5.54 (m, 1H), 5.49-5.42 (m, 1H), 5.32-5.26 (m, 1H), 5.23-5.16 (m, 1H), 4.77-4.69 (m, 2H), 4.32-4.26 (m, 2H), 3.94-3.87 (m, 3H), 3.80-3.74 (m, 1H), 3.59-3.53 (m, 1H), 3.49-3.44 (m, 2H), 3.31-3.31 (m, 3H), 3.19-3.12 (m, 1H), 3.01-2.95 (m, 1H), 2.90-2.84 (m, 3H), 2.45-2.40 (m, 3H), 2.36-2.29 (m, 1H), 2.23-2.17 (m, 1H), 1.88-1.82 (m, 1H), 1.75-1.69 (m, 1H), 1.48-1.43 (m, 3H), 1.22-1.19 (m, 2H), 0.94-0.90 (m, 3H), 0.43-0.33 (m, 2H), 0.07-0.01 (m, 1H), −0.01-−0.08 (m, 1H).

### Example 3-104: LD-104

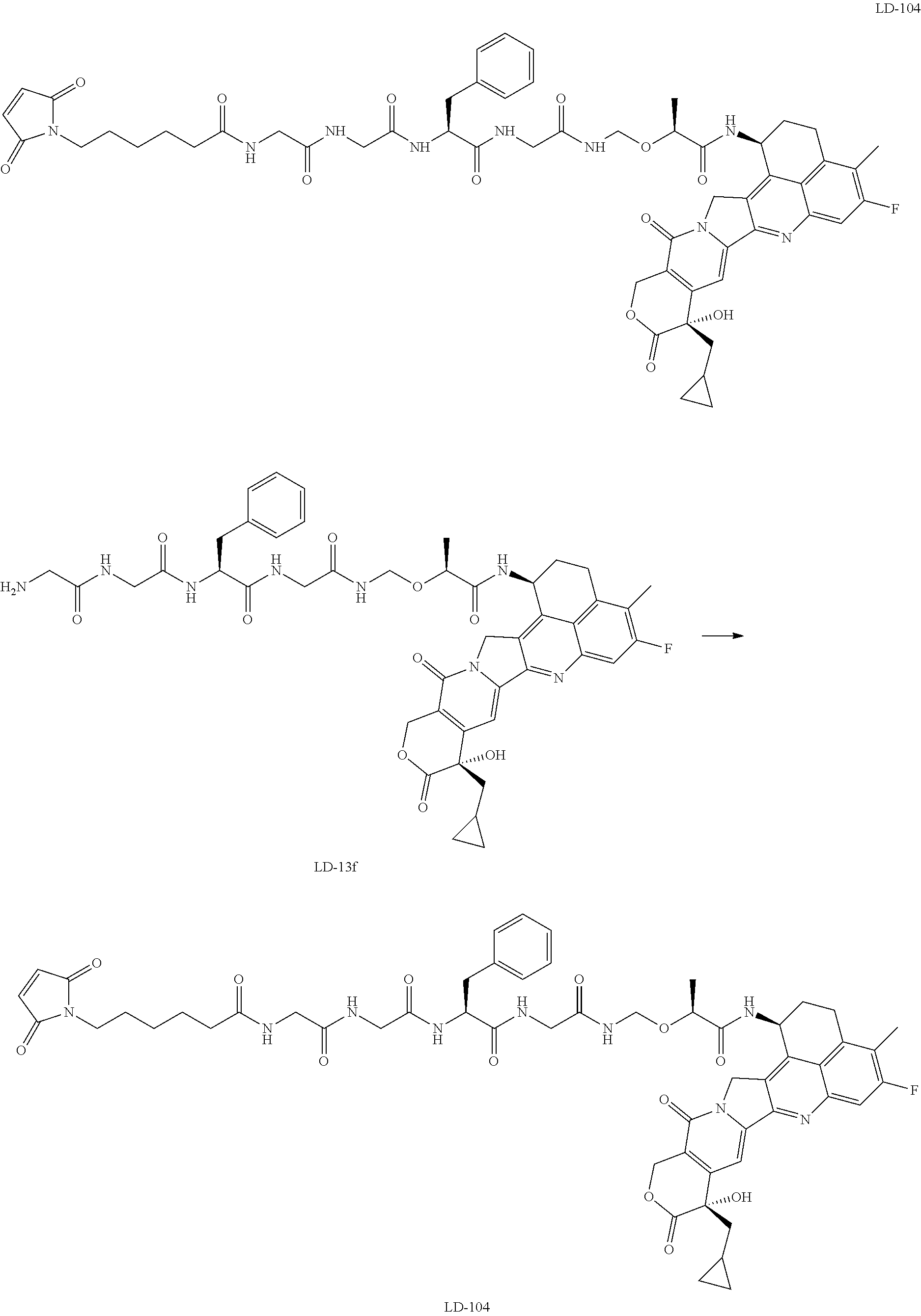 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CN)Cn1c-3cc2c(c1=O)COC(=O)[C@]2(O)CC1CC1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1]

LD-13f (18.0 mg, 20.4 μmol) and 6-(2,5-dioxopyrrol-1-yl)hexanoic acid (5.2 mg, 24.5 μmol) were dissolved in 1 mL of N,N-dimethylformamide, then added with 1-hydroxybenzotriazole (4.1 mg, 30.6 μmol), N,N-diisopropylethylamine (7.9 mg, 61.3 μmol) and 1-(3-dimethylaminopropyl)-3-ethylcarbodiimide hydrochloride (5.9 mg, 30.6 μmol) in sequence, and stirred at 25° C. for 1 hour.

The reaction solution was concentrated under reduced pressure, and the residue was purified by pre-HPLC (chromatographic column: Welch Xtimate C18 150×25 mm×5 μm; mobile phase: A-water (0.1% trifluoroacetic acid), B-acetonitrile; gradient elution: B %: 26%-56%) to obtain the title product LD-104 (3.94 mg, yield: 16%) as a yellow solid.

MS m/z (ESI): 1074.2 [M+1].

¹H NMR (400 MHz, CD3CN) δ 8.00-7.91 (m, 1H), 7.79-7.71 (m, 1H), 7.55-7.49 (m, 1H), 7.42-7.31 (m, 3H), 7.28-7.17 (m, 4H), 7.17-7.05 (m, 3H), 6.72-6.63 (m, 2H), 5.62-5.45 (m, 2H), 5.25-5.14 (m, 2H), 4.88-4.79 (m, 1H), 4.72-4.62 (m, 2H), 4.26-4.12 (m, 2H), 3.74-3.62 (m, 4H), 3.56-3.50 (m, 1H), 3.46-3.40 (m, 1H), 3.39-3.32 (m, 2H), 3.27-3.04 (m, 6H), 2.38-2.26 (m, 4H), 2.17-2.08 (m, 3H), 1.84-1.74 (m, 1H), 1.72-1.62 (m, 1H), 1.52-1.44 (m, 3H), 1.43-1.36 (m, 3H), 1.25-1.14 (m, 2H), 0.84-0.74 (m, 1H), 0.39-0.30 (m, 2H), 0.08-−0.10 (m, 2H).

### Example 3-105: LD-105

### N-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-6-(2,5-dioxo-2,5-dihydro-1H-pyrrol-1-yl)hexanamide LD-105

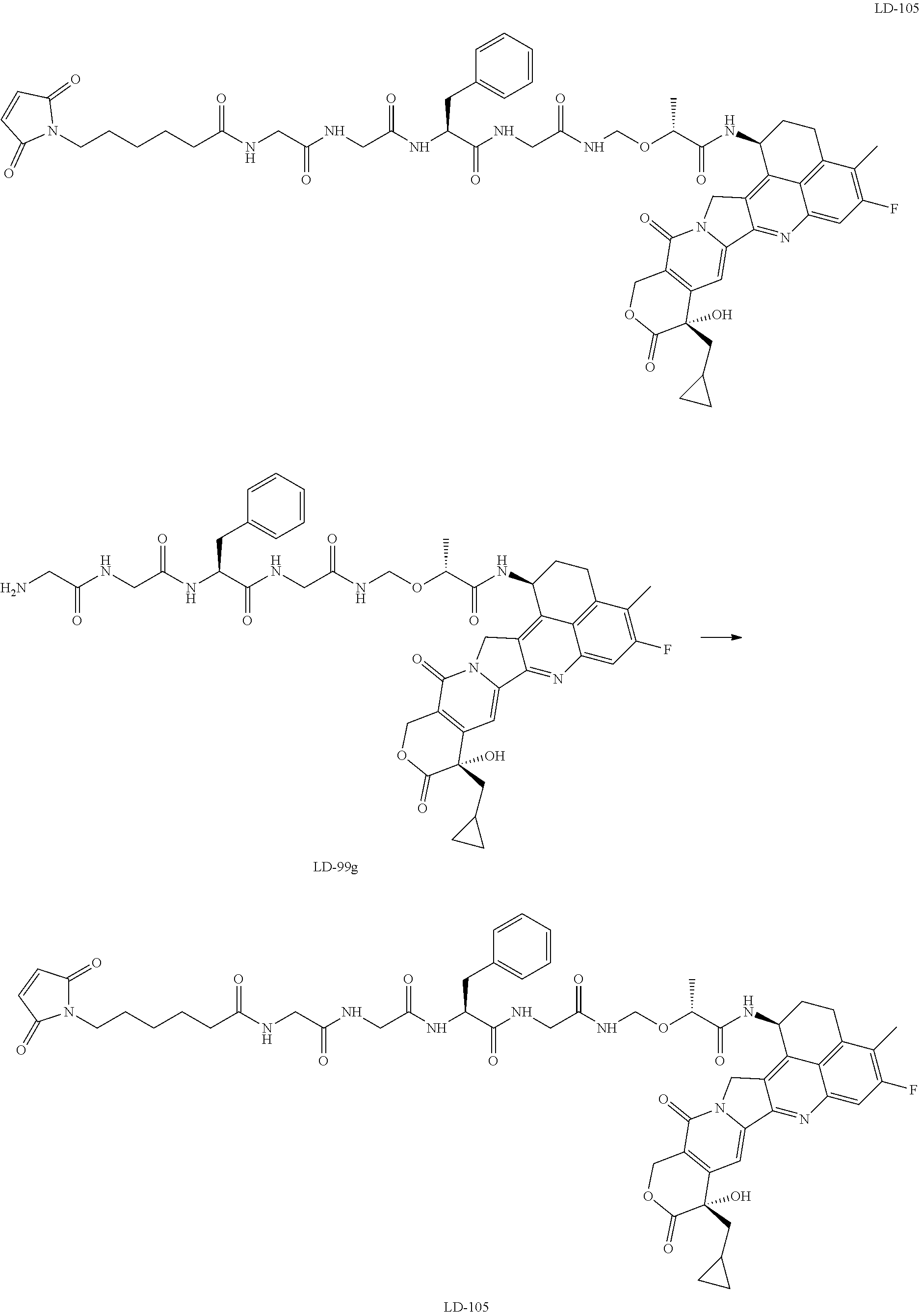 [SMILES: Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1 ; Cc1c(F)cc2nc3c(c4c2c1CC[C@@H]4NC(=O)[C@@H](C)OCNC(=O)CNC(=O)[C@H](Cc1ccccc1)NC(=O)CNC(=O)CN)Cn1c-3cc2c(c1=O)COC(=O)[C@]2(O)CC1CC1 ; Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1]

### N-((2R,10S)-10-Benzyl-1-(((1S,9S)-9-(cyclopropylmethyl)-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1,6,9,12,15-pentaoxo-3-oxa-5,8,11,14-tetraazahexadecan-16-yl)-6-(2,5-dioxo-2,5-dihydro-1H-pyrrol-1-yl)hexanamide LD-105

LD-99 g (30.0 mg, 34.1 μmol) and 6-(2,5-dioxopyrrol-1-yl)hexanoic acid (8.6 mg, 40.8 μmol) were dissolved in 0.5 mL of N,N-dimethylformamide, then added with 1-hydroxybenzotriazole (6.90 mg, 51.0 μmol), N,N-diisopropylethylamine (8.8 mg, 68.1 μmol) and 1-(3-dimethylaminopropyl)-3-ethylcarbodiimide hydrochloride (9.8 mg, 51.0 μmol) in sequence, and stirred at ° C. for 1 hour. 1 mL of water was added, dichloromethane (3 mL×2) was used for extraction, the organic phase was dried over anhydrous sodium sulfate and filtered, and the filtrate was concentrated by vacuum distillation. The resulting residue was purified by pre-HPLC (chromatographic column: Welch Xtimate C18 150×25 mm×5 μm; mobile phase: A-water (0.1% trifluoroacetic acid), B-acetonitrile; gradient elution: B %: 32%-52%) to obtain the title product LD-105 (3.17 mg, yield: 8%) as a white solid.

MS m/z (ESI): 1074.6 [M+1].

¹H NMR (400 MHz, DMSO-d6) δ 8.65-8.58 (m, 1H), 8.57-8.52 (m, 1H), 8.31-8.26 (m, 1H), 8.11-7.97 (m, 3H), 7.80-7.74 (m, 1H), 7.39-7.35 (m, 1H), 7.26-7.13 (m, 5H), 7.01-6.93 (m, 2H), 6.59-6.55 (m, 1H), 5.63-5.55 (m, 1H), 5.44-5.36 (m, 2H), 5.27-5.18 (m, 1H), 5.14-5.06 (m, 1H), 4.72-4.66 (m, 1H), 4.57-4.51 (m, 1H), 4.48-4.42 (m, 1H), 4.16-4.09 (m, 1H), 3.76-3.63 (m, 5H), 3.61-3.54 (m, 1H), 3.17-3.09 (m, 2H), 3.04-2.95 (m, 2H), 2.78-2.69 (m, 2H), 2.41-2.36 (m, 3H), 2.26-2.15 (m, 3H), 2.11-2.05 (m, 2H), 1.82-1.74 (m, 2H), 1.48-1.39 (m, 6H), 1.19-1.13 (m, 2H), 0.85-0.77 (m, 1H), 0.37-0.28 (m, 2H), 0.09-0.02 (m, 1H), −0.04-−0.12 (m, 1H).

### Example 3-106: LD-106

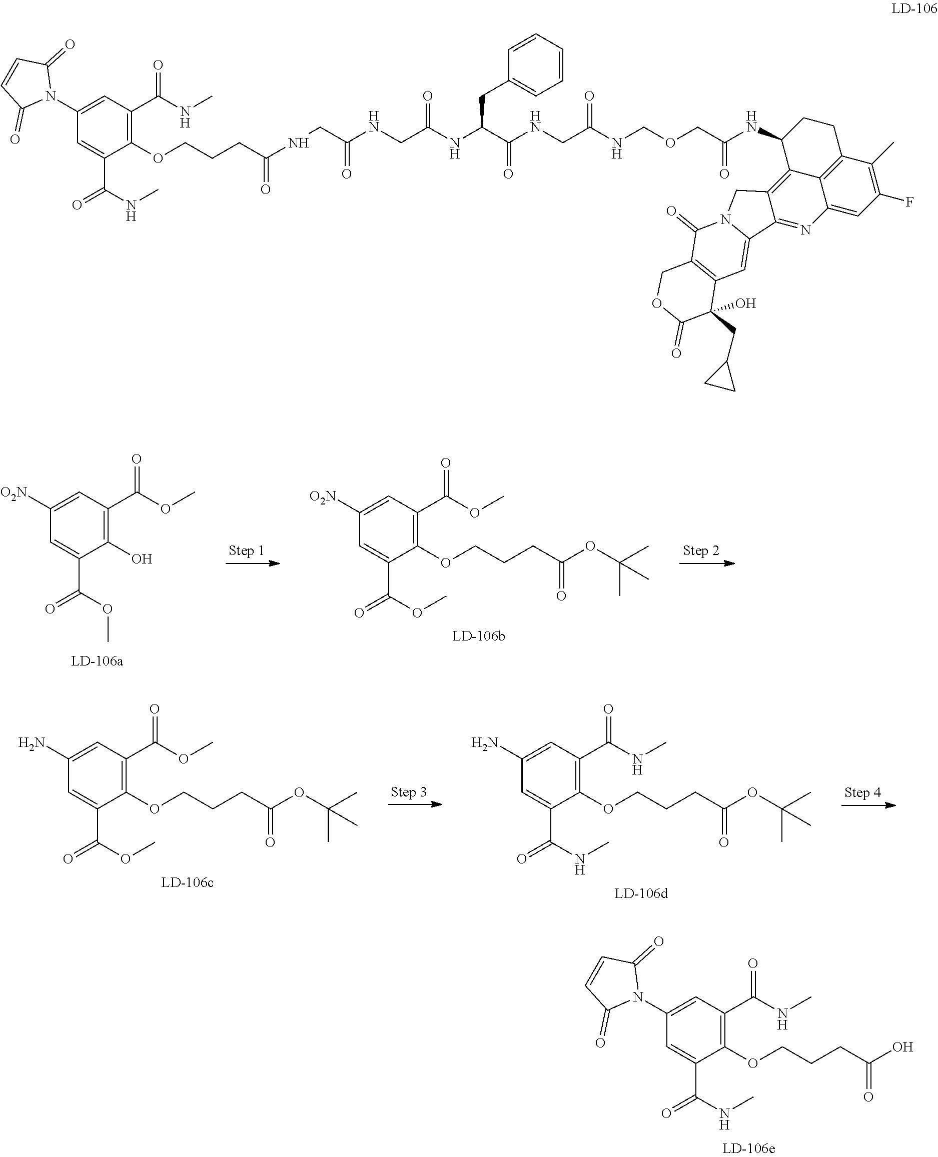 [SMILES: CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCOCC(=O)N[C@H]1CCc2c(C)c(F)cc3nc4c(c1c23)Cn1c-4cc2c(c1=O)COC(=O)[C@]2(O)CC1CC1 ; COC(=O)c1cc([N+](=O)[O-])cc(C(=O)OC)c1O ; COC(=O)c1cc([N+](=O)[O-])cc(C(=O)OC)c1OCCCC(=O)OC(C)(C)C ; COC(=O)c1cc(N)cc(C(=O)OC)c1OCCCC(=O)OC(C)(C)C ; CNC(=O)c1cc(N)cc(C(=O)NC)c1OCCCC(=O)OC(C)(C)C ; CNC(=O)c1cc(N2C(=O)C=CC2=O)cc(C(=O)NC)c1OCCCC(=O)O]

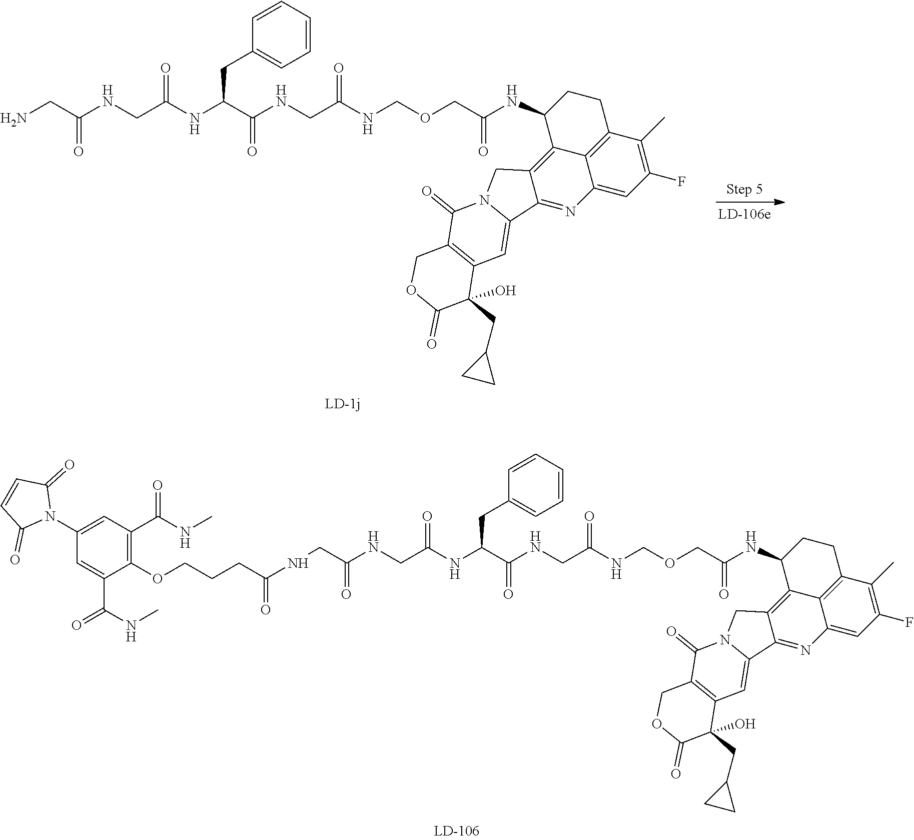 [SMILES: Cc1c(F)cc2nc3c(c4c2c1CC[C@@H]4NC(=O)COCNC(=O)CNC(=O)[C@H](Cc1ccccc1)NC(=O)CNC(=O)CN)Cn1c-3cc2c(c1=O)COC(=O)[C@]2(O)CC1CC1 ; CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCOCC(=O)N[C@H]1CCc2c(C)c(F)cc3nc4c(c1c23)Cn1c-4cc2c(c1=O)COC(=O)[C@]2(O)CC1CC1]

### Step 1

LD-106a (5 g, 19.55 mmol, prepared by applying the method disclosed in Reference Example 121 on page 287 of patent application “CN1572882A”), tert-butyl 4-hydroxybutyrate (3.76 g, 23.47 mmol) and triphenylphosphine (7.69 g, 29.33 mmol) were dissolved in 40 mL of N,N-dimethylformamide and 120 ml, tetrahydrofuran mixed solvent, the reaction solution was cooled to 0° C., then diisopropyl azodicarboxylate (5.93 g, 29.33 mmol) was slowly added dropwise, replacement with nitrogen was performed three times, and the reaction mixture was stirred at 25° C. for 20 hours under nitrogen protection. 150 mL of water was added, ethyl acetate (200 mL×3) was used for extraction, the organic phase was dried over anhydrous sodium sulfate and filtered, and the filtrate was concentrated by vacuum distillation. The residue was purified by reverse phase high performance liquid chromatography (chromatographic column: I.D.100 mm×H300 mm Welch Ultimate XB_C18 20-40 μm; mobile phase: A-water, B-acetonitrile; isocratic elution: B %: 78%, instrument: Agela Astra) to obtain the title product LD-106b (4.8 g, yield: 61%) as a yellow solid.

MS m/z (ESI): 420.2 [M+23].

### Step 2

LD-106b (3.50 g, 8.65 mmol) was dissolved in 30 mL of methanol and 10 mL of water, added with iron powder (2.42 g, 43.3 mmol) and ammonium chloride (2.31 g, 43.3 mmol) in sequence at 25° C., and stirred at 80° C. for 2 hours. The reaction solution was filtered, and the filtrate was extracted with dichloromethane (50 mL×2). The organic phase was washed with saturated sodium chloride solution (50 mL×2), then dried over anhydrous sodium sulfate and filtered, and the filtrate was concentrated by vacuum distillation to obtain the crude title product LD-106c (2.80 g) as a yellow oil. The product was used directly in the next step without purification.

MS m/z (ESI): 390.0 [M+23].

### Step 3

LD-106c (2.80 g, 7.46 mmol) was added to a methanol solution of methylamine (10.5 g, purity 30%), and stirred at 50° C. for 12 hours. The reaction solution was concentrated by vacuum distillation to obtain the crude title product LD-106d (2.67 g) as a yellow solid. The product was used directly in the next reaction without purification.

MS m/z (ESI): 310.0 [M+1-56].

### Step 4

LD-106d (200 mg, 518 μmol) and 2,5-furandione (76.0 mg, 775 μmol) were dissolved in 5 mL of dioxane, and stirred at 25° C. for 1 hour. Dimethyl sulfoxide (80.8 mg, 1.03 mmol) and ammonium persulfate (236 mg, 1.03 mmol) were added at 25° C., and stirred at 100° C. for 12 hours. The reaction solution was concentrated under reduced pressure, and the residue was purified by pre-HPLC (chromatographic column: Welch Xtimate C18 150×40 mm×10 μm; mobile phase: A-water (0.1% trifluoroacetic acid), B-acetonitrile; gradient elution: B %: 0%-28%) to obtain the title product LD-106e (50.0 mg, yield: 23%) as a yellow solid.

MS m/z (ESI): 390.0 [M+1].

### Step 5

LD-106e (10.8 mg, 27.7 μmol) and LD-1j (20.0 mg, 23.1 μmol) were dissolved in 1 mL of N,N-dimethylformamide, then added with 1-hydroxybenzotriazole (4.68 mg, 34.6 μmol), N,N-diisopropylethylamine (8.95 mg, 69.2 μmol) and 1-(3-dimethylaminopropyl)-3-ethylcarbodiimide hydrochloride (6.63 mg, 34.6 μmol) in sequence, and stirred at 25° C. for 1 hour. The reaction solution was concentrated by vacuum distillation, and the resulting residue was purified by pre-HPLC (chromatographic column: Welch Xtimate C18 150×25 mm×5 μm; mobile phase: A-water (0.1% trifluoroacetic acid), B-acetonitrile; gradient elution: B %: 28%-48%) to obtain the title product LD-106 (15.8 mg, yield: 57%) as a white solid.

MS m/z (ESI): 1238.3 [M+1].

¹H NMR (400 MHz, DMSO-d6) δ 8.66-8.60 (m, 1H), 8.55-8.49 (m, 1H), 8.33-8.23 (m, 3H), 8.16-8.09 (m, 2H), 8.06-8.02 (m, 1H), 7.81-7.76 (m, 1H), 7.53-7.48 (m, 2H), 7.39-7.36 (m, 1H), 7.27-7.14 (m, 7H), 6.63-6.52 (m, 1H), 5.63-5.55 (m, 1H), 5.44-5.38 (m, 2H), 5.24-5.18 (m, 2H), 4.68-4.61 (m, 2H), 4.50-4.43 (m, 1H), 4.05-3.99 (m, 2H), 3.94-3.89 (m, 2H), 3.78-3.74 (m, 1H), 3.72-3.68 (m, 4H), 3.62-3.59 (m, 1H), 3.20-3.11 (m, 2H), 3.05-2.98 (m, 1H), 2.83-2.79 (m, 1H), 2.79-2.74 (m, 6H), 2.41-2.36 (m, 3H), 2.30-2.24 (m, 2H), 2.21-2.13 (m, 2H), 1.92-1.85 (m, 2H), 1.84-1.79 (m, 1H), 1.78-1.71 (m, 1H), 0.85-0.77 (m, 1H), 0.37-0.27 (m, 2H), 0.08-0.03 (m, 1H), −0.06-−0.11 (m, 1H).

### Example 3-107: LD-107

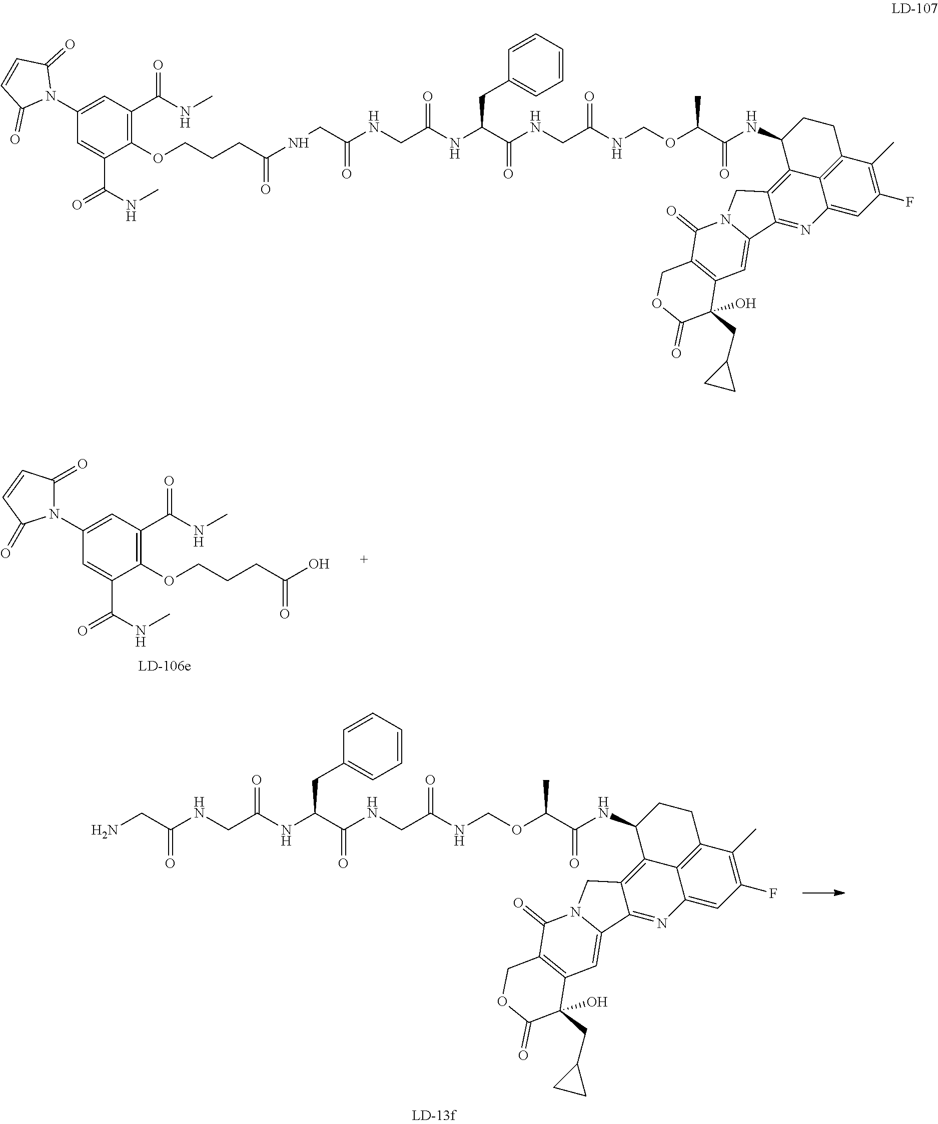 [SMILES: CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@@H](C)C(=O)N[C@H]1CCc2c(C)c(F)cc3nc4c(c1c23)Cn1c-4cc2c(c1=O)COC(=O)[C@]2(O)CC1CC1 ; CNC(=O)c1cc(N2C(=O)C=CC2=O)cc(C(=O)NC)c1OCCCC(=O)O ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CN)Cn1c-3cc2c(c1=O)COC(=O)[C@]2(O)CC1CC1]

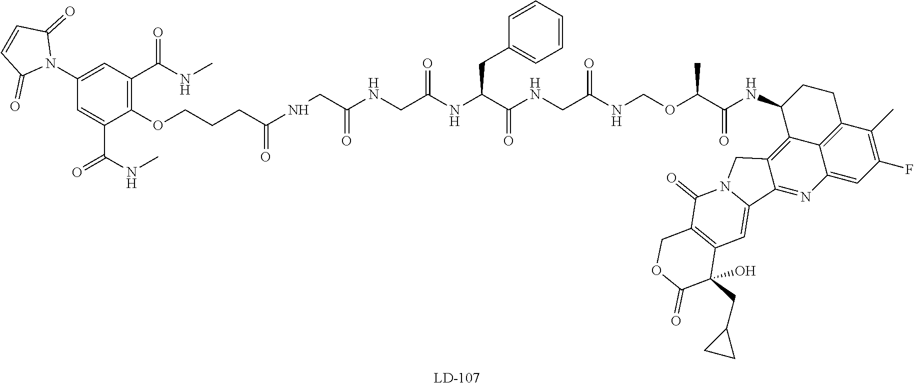 [SMILES: CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@@H](C)C(=O)N[C@H]1CCc2c(C)c(F)cc3nc4c(c1c23)Cn1c-4cc2c(c1=O)COC(=O)[C@]2(O)CC1CC1]

LD-106e (10.6 mg, 25.6 μmol) and LD-13f (20.0 mg, 21.3 μmol) were dissolved in 1 mL of N,N-dimethylformamide, then added with 1-hydroxybenzotriazole (4.3 mg, 32.1 μmol), N,N-diisopropylethylamine (8.3 mg, 64.1 μmol) and 1-(3-dimethylaminopropyl)-3-ethylcarbodiimide hydrochloride (6.2 mg, 32.1 μmol) in sequence, and stirred at 25° C. for 1 hour. The reaction solution was concentrated by vacuum distillation, and the residue was purified by pre-HPLC (chromatographic column: Welch Xtimate C18 150×25 mm×5 μm; mobile phase: A-water (0.1% trifluoroacetic acid), B-acetonitrile; gradient elution: B %: 32%-52%) to obtain the title product LD-107 (4.23 mg, yield: 15%) as a yellow solid.

MS m/z (ESI): 1252.6 [M+1].

¹H NMR (400 MHz, DMSO-d6) δ 8.67-8.62 (m, 1H), 8.57-8.52 (m, 1H), 8.30-8.21 (m, 3H), 8.15-8.08 (m, 2H), 8.04-8.00 (m, 1H), 7.80-7.75 (m, 1H), 7.54-7.48 (m, 2H), 7.41-7.36 (m, 1H), 7.28-7.12 (m, 7H), 6.60-6.56 (m, 1H), 5.57-5.52 (m, 1H), 5.44-5.37 (m, 2H), 5.26-5.19 (m, 2H), 4.74-4.69 (m, 1H), 4.61-4.56 (m, 1H), 4.48-4.43 (m, 1H), 4.15-4.09 (m, 1H), 3.95-3.88 (m, 2H), 3.73-3.64 (m, 4H), 3.61-3.55 (m, 1H), 3.17-3.12 (m, 2H), 3.02-2.98 (m, 1H), 2.82-2.72 (m, 6H), 2.40-2.36 (m, 3H), 2.29-2.25 (m, 2H), 2.19-2.14 (m, 2H), 1.91-1.80 (m, 4H), 1.76-1.70 (m, 2H), 1.34-1.29 (m, 3H), 0.83-0.79 (m, 1H), 0.36-0.28 (m, 2H), 0.07-0.03 (m, 1H), −0.06-−0.10 (m, 1H).

### Example 3-108: LD-108

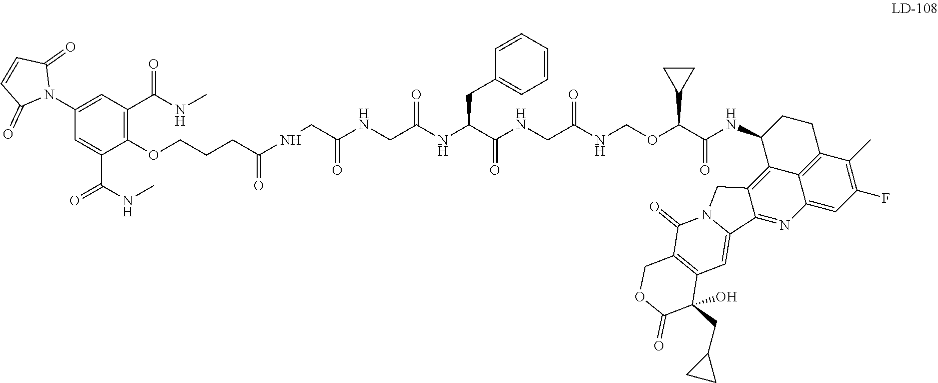 [SMILES: CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@H](C(=O)N[C@H]1CCc2c(C)c(F)cc3nc4c(c1c23)Cn1c-4cc2c(c1=O)COC(=O)[C@]2(O)CC1CC1)C1CC1]

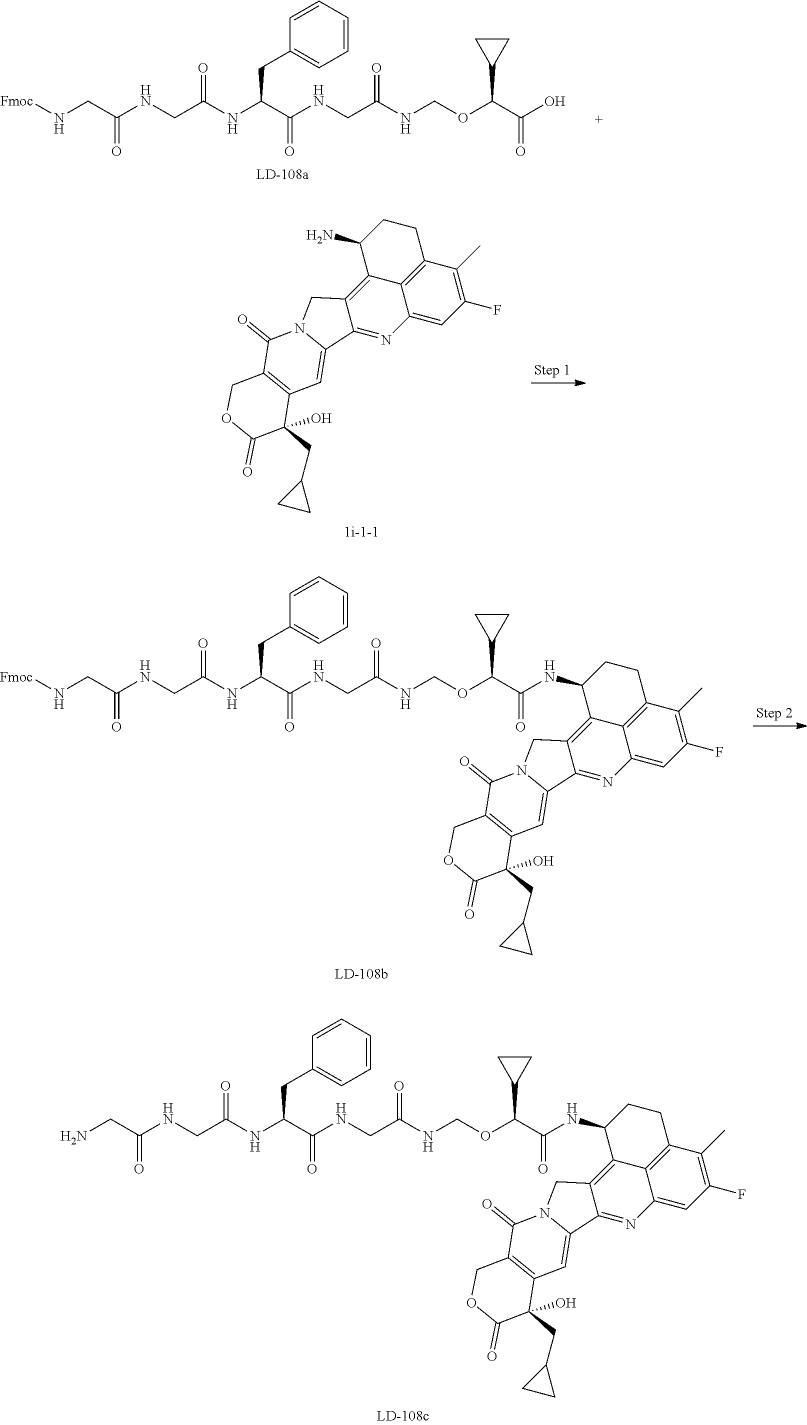

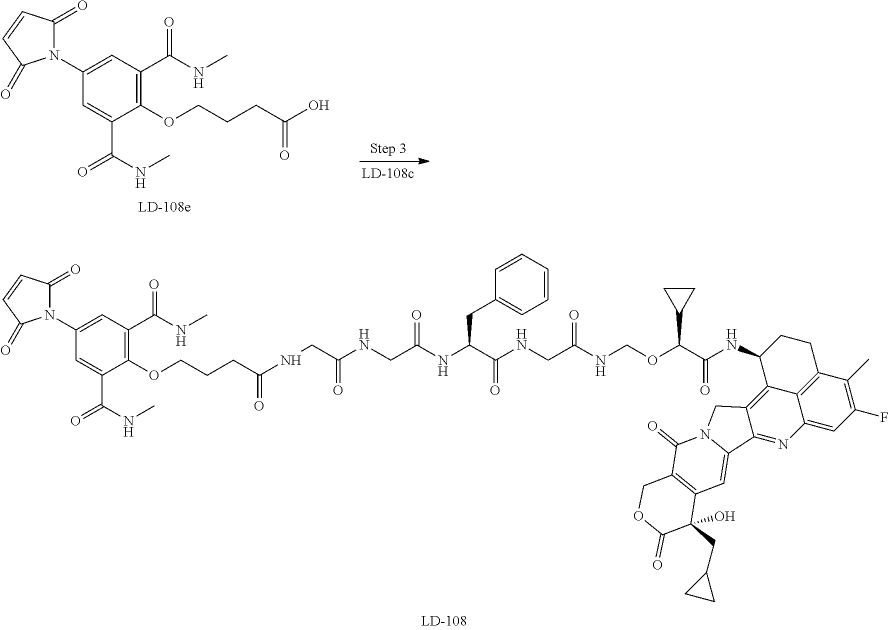 [SMILES: CNC(=O)c1cc(N2C(=O)C=CC2=O)cc(C(=O)NC)c1OCCCC(=O)O ; CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@H](C(=O)N[C@H]1CCc2c(C)c(F)cc3nc4c(c1c23)Cn1c-4cc2c(c1=O)COC(=O)[C@]2(O)CC1CC1)C1CC1]

Step 1 LD-108a (100 mg, 126 μmol, prepared by applying the method disclosed in Example 1-19 on page 65 of patent application “WO2021190602 A1”) and 1i-1-1 (70.5 mg, 126 μmol, HCl) were dissolved in 5 mL of N,N-dimethylformamide, then added with 1-hydroxybenzotriazole (25.7 mg, 190 μmol), N,N-diisopropylethylamine (49.2 mg, 380 μmol) and 1-(3-dimethylaminopropyl)-3-ethylcarbodiimide hydrochloride (36.5 mg, 190 μmol) in sequence, and stirred at 25° C. for 1 hour. 10 mL of water was added to the reaction solution, dichloromethane (15 mL×3) was used for extraction, the organic phase was dried over anhydrous sodium sulfate and filtered, and the filtrate was concentrated by vacuum distillation. The obtained residue was purified by silica gel column chromatography using the eluent system A to obtain the title product LD-108b (103 mg, yield: 54%) was obtained as a white solid.

MS m/z (ESI): 1129.2 [M+1].

### Step 2

LD-108b (102 mg, 67.7 μmol) was dissolved in 4 mL of acetonitrile, added with diethylamine (1.0 mL), and stirred at 25° C. for 1 hour. The reaction solution was concentrated by vacuum distillation, and the obtained residue was purified by pre-HPLC (chromatographic column: Welch Xtimate C18 150×25 mm×5 μm; mobile phase: A-water (0.1% trifluoroacetic acid), B-acetonitrile; gradient elution: B %: 22%-42%) to obtain the title product LD-108c (32.0 mg, yield: 49%) as a yellow solid.

MS m/z (ESI): 907.2 [M+1].

### Step 3

LD-106e (6.88 mg, 16.2 μmol) and LD-108c (15.9 mg, 14.7 μmol, trifluoroacetate) were dissolved in 1 mL of N,N-dimethylformamide, then added with 1-hydroxybenzotriazole (2.98 mg, 22.0 μmol), N,N-diisopropylethylamine (5.70 mg, 44.1 μmol) and 1-(3-dimethylaminopropyl)-3-ethylcarbodiimide hydrochloride (4.22 mg, 22.0 μmol) in sequence, and stirred at 25° C. for 1 hour. 50 mL of water was added to the reaction solution, and dichloromethane (45 mL×3) was used for extraction. The organic phase was dried over anhydrous sodium sulfate and filtered, and the filtrate was concentrated by vacuum distillation. The residue was purified by pre-HPLC (chromatographic column: Phenomenex luna C18 150×25 mm×10 μm; mobile phase: A-water (0.225% formic acid), B-acetonitrile; gradient elution: B %: 28%-58%) to obtain the title product LD-108 (4.22 mg, yield: 22%) as a white solid.

MS m/z (ESI): 1278.3 [M+1].

¹H NMR (400 MHz, CD3CN) δ 8.03-7.96 (m, 1H), 7.67-7.63 (m, 2H), 7.60-7.44 (m, 4H), 7.40-7.31 (m, 2H), 7.30-7.05 (m, 7H), 6.93-6.85 (m, 2H), 5.61-5.46 (m, 2H), 5.30-5.18 (m, 2H), 4.91-4.80 (m, 2H), 4.78-4.71 (m, 1H), 4.66-4.60 (m, 1H), 4.24-4.15 (m, 1H), 3.96-3.86 (m, 2H), 3.80-3.67 (m, 3H), 3.64-3.51 (m, 3H), 3.49-3.39 (m, 1H), 3.25-3.15 (m, 1H), 3.14-3.03 (m, 1H), 3.00-2.91 (m, 1H), 2.91-2.81 (m, 6H), 2.81-2.75 (m, 1H), 2.44-2.31 (m, 5H), 2.30-2.22 (m, 2H), 2.02-1.97 (m, 2H), 1.86-1.75 (m, 2H), 1.72-1.65 (m, 1H), 1.16-1.08 (m, 1H), 0.84-0.75 (m, 1H), 0.62-0.45 (m, 4H), 0.40-0.31 (m, 2H), 0.07-−0.06 (m, 2H).

### Example 3-109: LD-109

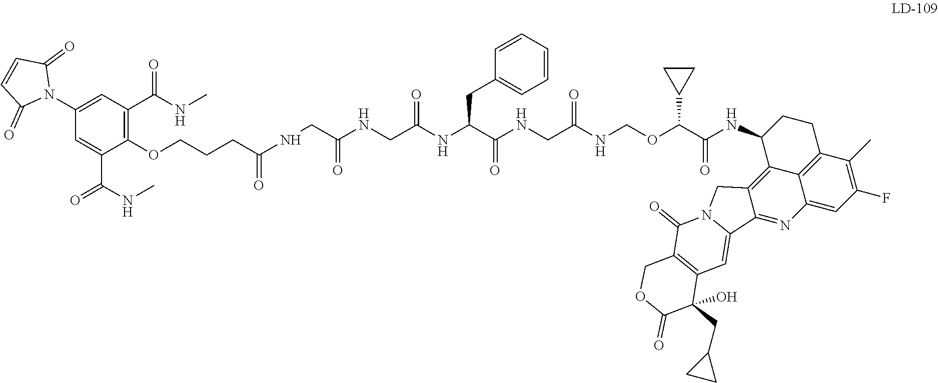 [SMILES: CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@@H](C(=O)N[C@H]1CCc2c(C)c(F)cc3nc4c(c1c23)Cn1c-4cc2c(c1=O)COC(=O)[C@]2(O)CC1CC1)C1CC1]

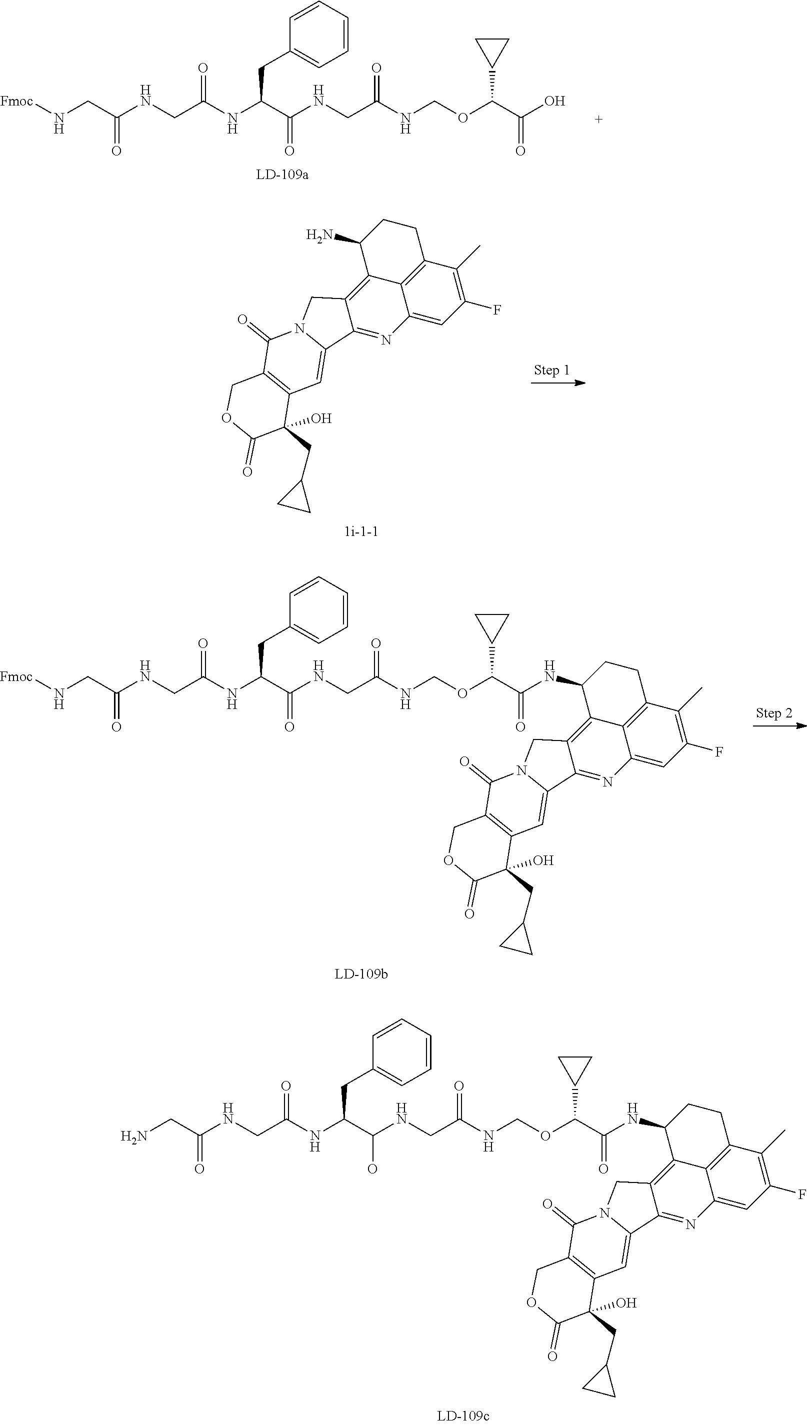

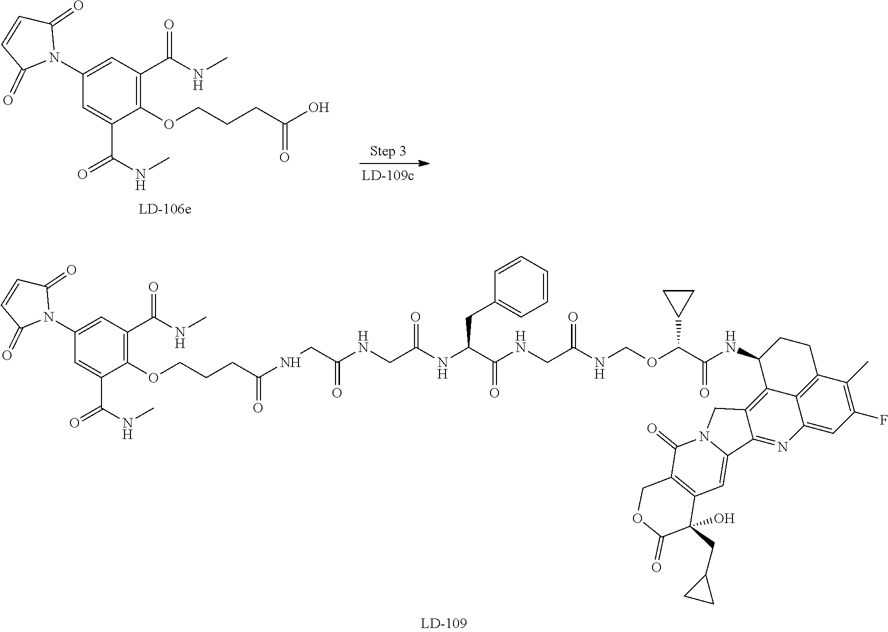 [SMILES: CNC(=O)c1cc(N2C(=O)C=CC2=O)cc(C(=O)NC)c1OCCCC(=O)O ; CNC(=O)c1cc(N2C(=O)C=CC2=O)cc(C(=O)NC)c1OCCCC(=O)NCC(=O)NCC(=O)N[C@@H](Cc1ccccc1)C(=O)NCC(=O)NCO[C@@H](C(=O)N[C@H]1CCc2c(C)c(F)cc3nc4c(c1c23)Cn1c-4cc2c(c1=O)COC(=O)[C@]2(O)CC1CC1)C1CC1]

### Step 1

LD-109a (135 mg, 197 μmol, prepared by applying the method disclosed in Example 1-18 on page 61 of patent application “WO2021190602 A1”) and 1i-1-1 (100 mg, 179 μmol, HCl) were dissolved in 3 mL of N,N-dimethylformamide, then added with 1-hydroxybenzotriazole (36.2 mg, 268 μmol), N,N-diisopropylethylamine (69.3 mg, 536 μmol) and 1-(3-dimethylaminopropyl)-3-ethylcarbodiimide hydrochloride (51.4 mg, 268 μmol) in sequence, and stirred at 25° C. for 2 hours. 5 mL of water was added to the reaction solution, dichloromethane (5 mL×3) was used for extraction, the organic phase was dried over anhydrous sodium sulfate and filtered, and the filtrate was concentrated by vacuum distillation. The obtained residue was purified by silica gel column chromatography with the eluent system A to obtain the title product LD-109b (180 mg, yield: 77%) as a yellow solid.

MS m/z (ESI): 1129.6 [M+1].

### Step 2

LD-109b (100 mg, 88.5 μmol) was dissolved in 2 mL of acetonitrile, added with diethylamine (284 mg, 3.88 mmol), and stirred at 25° C. for 1 hour. The reaction solution was concentrated by vacuum distillation, and the residue was purified by pre-HPLC (chromatographic column: Welch Xtimate C18 150×25 mm×5 μm; mobile phase: A-water (0.1% trifluoroacetic acid), B-acetonitrile; gradient elution: B %: 40%-60%) to obtain the title product LD-109c (45.0 mg, yield: 54%) as a yellow solid.

MS m/z (ESI): 907.5 [M+1].

### Step 3

LD-106e (10.7 mg, 25.3 μmol) and LD-109c (20.0 mg, 21.1 μmol) were dissolved in 1 mL of N,N-dimethylformamide, then added with 1-hydroxybenzotriazole (4.3 mg, 31.6 μmol), N,N-diisopropylethylamine (8.2 mg, 63.3 μmol) and 1-(3-dimethylaminopropyl)-3-ethylcarbodiimide hydrochloride (6.1 mg, 31.6 μmol) in sequence, and stirred at 25° C. for 2 hours. 3 mL of water was added to the reaction solution, dichloromethane (5 mL×2) was used for extraction, the organic phase was dried over anhydrous sodium sulfate and filtered, and the filtrate was concentrated by vacuum distillation. The residue was purified by pre-HPLC (chromatographic column: Phenomenex luna C18 150×25 mm×10 μm; mobile phase: A-water (0.225% formic acid), B-acetonitrile; gradient elution: B %: 24%-54%) to obtain the title product LD-109 (11 mg, yield: 40%) as a white solid.

MS m/z (ESI): 1278.6 [M+1].

¹H NMR (400 MHz, CD3CN) δ 7.99-7.94 (m, 1H), 7.86-7.81 (m, 1H), 7.68-7.64 (m, 2H), 7.62-7.54 (m, 2H), 7.51-7.45 (m, 2H), 7.37-7.34 (m, 1H), 7.32-7.27 (m, 1H), 7.24-7.14 (m, 5H), 7.11-7.06 (m, 2H), 6.93-6.88 (m, 2H), 5.61-5.56 (m, 1H), 5.53-5.48 (m, 1H), 5.31-5.24 (m, 2H), 4.99-4.93 (m, 1H), 4.84-4.79 (m, 1H), 4.58-4.56 (m, 1H), 4.54-4.49 (m, 1H), 4.24-4.19 (m, 1H), 3.95-3.89 (m, 2H), 3.73-3.69 (m, 2H), 3.65-3.61 (m, 1H), 3.55-3.51 (m, 3H), 3.28-3.23 (m, 1H), 3.14-3.09 (m, 1H), 3.03-2.98 (m, 1H), 2.91-2.82 (m, 6H), 2.82-2.77 (m, 1H), 2.40-2.34 (m, 5H), 2.28-2.24 (m, 2H), 2.01-1.99 (m, 2H), 1.85-1.82 (m, 1H), 1.78-1.73 (m, 2H), 1.22-1.18 (m, 1H), 0.87-0.82 (m, 1H), 0.59-0.51 (m, 3H), 0.44-0.37 (m, 3H), 0.08-0.01 (m, 2H).

### Example 3-110: LD-110

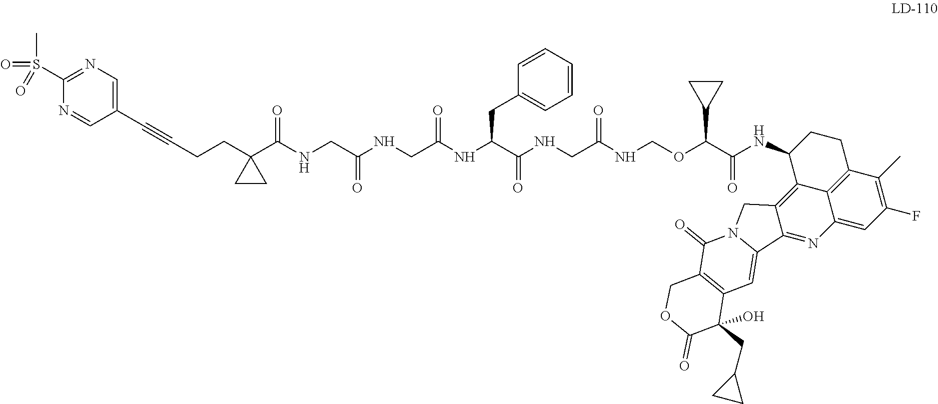 [SMILES: Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1]

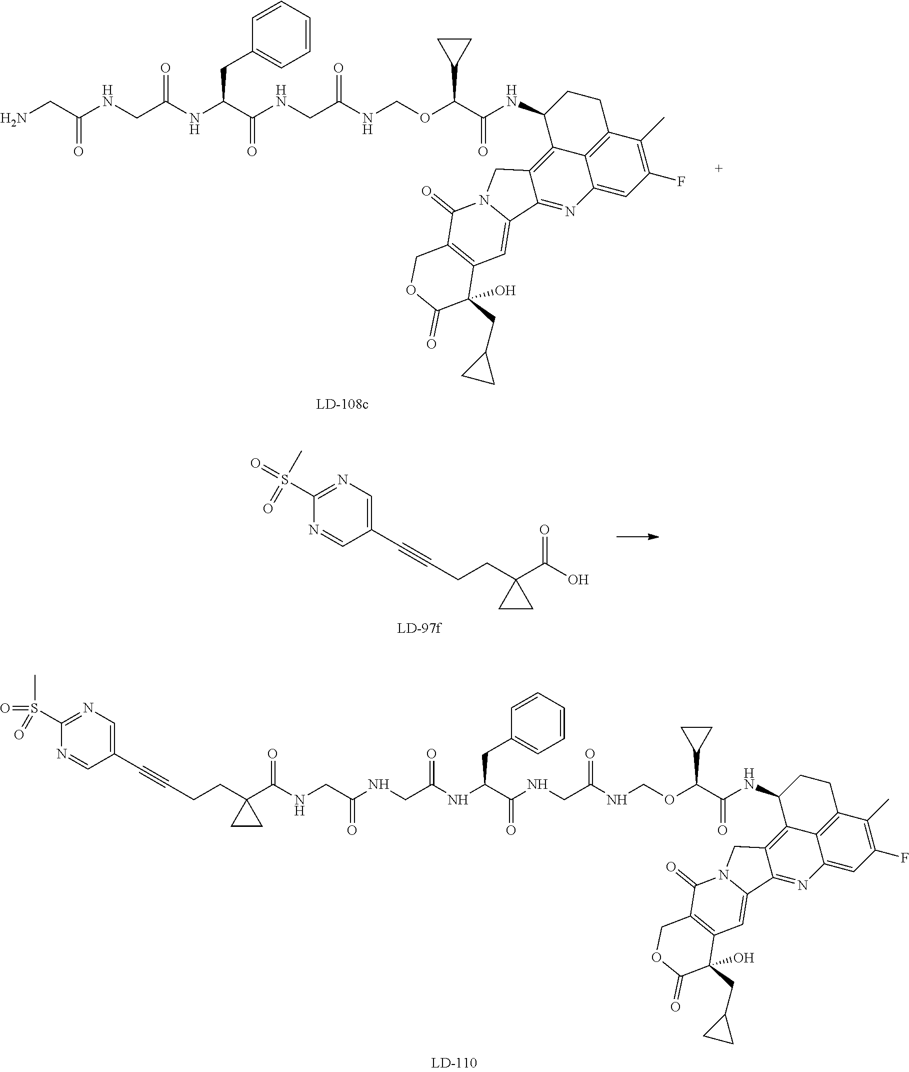 [SMILES: Cc1c(F)cc2nc3c(c4c2c1CC[C@@H]4NC(=O)[C@@H](OCNC(=O)CNC(=O)[C@H](Cc1ccccc1)NC(=O)CNC(=O)CN)C1CC1)Cn1c-3cc2c(c1=O)COC(=O)[C@]2(O)CC1CC1 ; CS(=O)(=O)c1ncc(C#CCCC2(C(=O)O)CC2)cn1 ; Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1]

LD-97f (4.55 mg, 16.2 μmol) and LD-108c (15.0 mg, 14.7 μmol, trifluoroacetate) were dissolved in 1 mL of N,N-dimethylformamide, then added with 1-hydroxybenzotriazole (2.98 mg, 22.0 μmol), N,N-diisopropylethylamine (5.70 mg, 44.1 μmol) and 1-(3-dimethylaminopropyl)-3-ethylcarbodiimide hydrochloride (4.22 mg, 22.0 μmol) in sequence, and stirred at 25° C. for 1 hour. 50 mL of water was added to the reaction solution, and dichloromethane (45 mL×3) was used for extraction. The organic phase was dried over anhydrous sodium sulfate and filtered, and the filtrate was concentrated by vacuum distillation. The residue was purified by pre-HPLC (chromatographic column: Phenomenex Kinetex EVO C18 150×30 mm×5 μm; mobile phase: A-water (0.225% formic acid), B-acetonitrile; gradient elution: B %: 37%-57%) to obtain the title product LD-110 (5.3 mg, yield: 31%) as a white solid.

MS m/z (ESI): 1183.2 [M+1].

¹H NMR (400 MHz, CD₃OD) δ 8.91-8.82 (m, 2H), 7.68-7.58 (m, 2H), 7.27-7.10 (m, 5H), 5.71-5.64 (m, 1H), 5.61-5.54 (m, 1H), 5.53-5.45 (m, 1H), 5.34-5.28 (m, 1H), 5.21-5.13 (m, 2H), 4.78-4.74 (m, 1H), 4.36-4.30 (m, 1H), 3.91-3.71 (m, 6H), 3.65-3.58 (m, 2H), 3.35-3.34 (m, 3H), 3.22-3.16 (m, 1H), 2.96-2.87 (m, 2H), 2.72-2.65 (m, 2H), 2.46-2.39 (m, 3H), 2.37-2.31 (m, 1H), 2.27-2.19 (m, 1H), 2.02-1.92 (m, 2H), 1.90-1.83 (m, 1H), 1.79-1.71 (m, 1H), 1.26-1.10 (m, 4H), 0.86-0.80 (m, 2H), 0.66-0.51 (m, 4H), 0.45-0.33 (m, 2H), 0.09-0.03 (m, 1H), 0.01-−0.07 (m, 1H).

### Example 3-111: LD-111

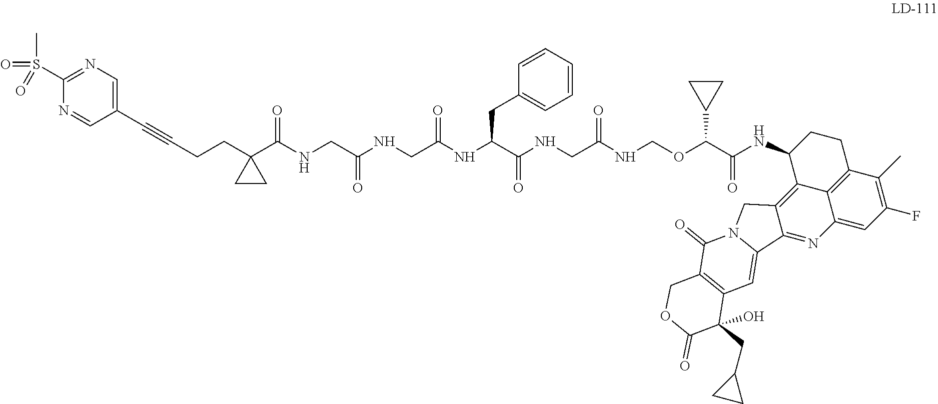 [SMILES: Cc1c(F)cc2nc3c(c4c2c1CC[C@@H]4NC(=O)[C@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1]

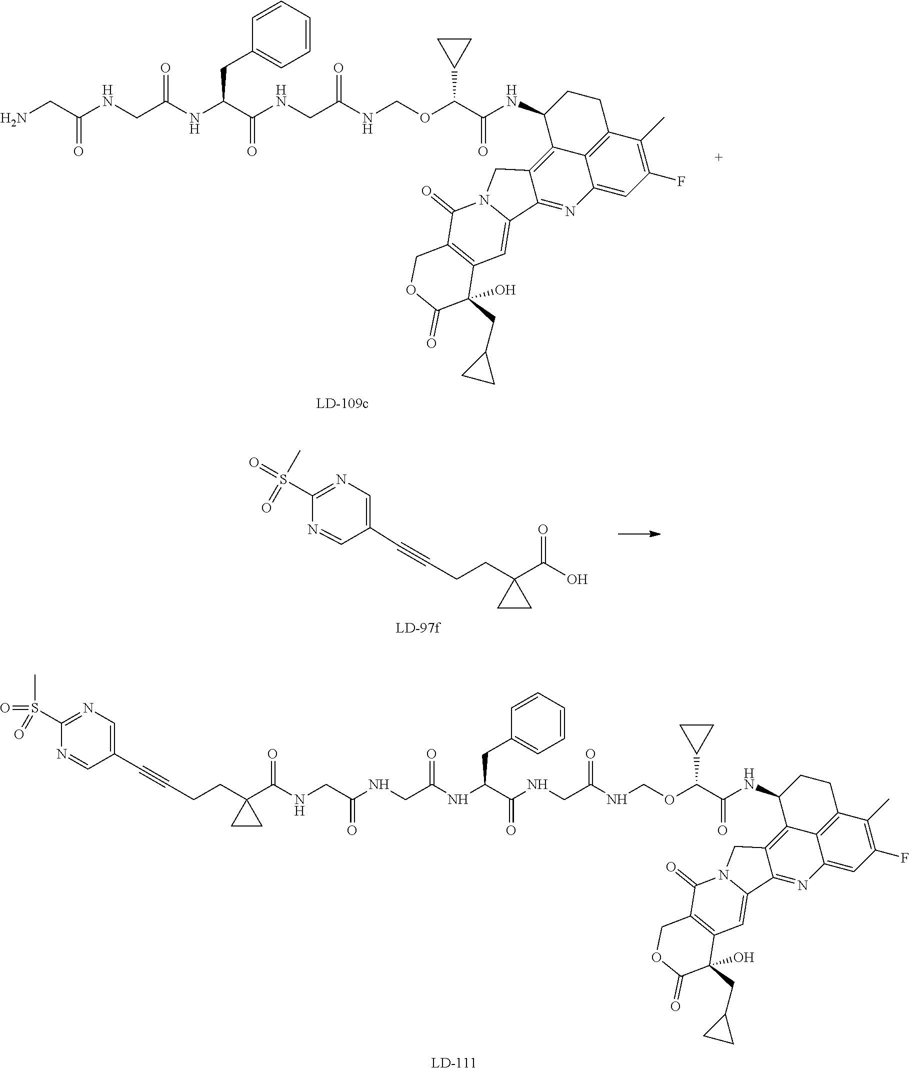 [SMILES: Cc1c(F)cc2nc3c(c4c2c1CC[C@@H]4NC(=O)[C@H](OCNC(=O)CNC(=O)[C@H](Cc1ccccc1)NC(=O)CNC(=O)CN)C1CC1)Cn1c-3cc2c(c1=O)COC(=O)[C@]2(O)CC1CC1 ; CS(=O)(=O)c1ncc(C#CCCC2(C(=O)O)CC2)cn1 ; Cc1c(F)cc2nc3c(c4c2c1CC[C@@H]4NC(=O)[C@H](OCNC(=O)CNC(=O)[C@H](Cc1ccccc1)NC(=O)CNC(=O)CNC(=O)C1(CCC#Cc2cnc(S(C)(=O)=O)nc2)CC1)C1CC1)Cn1c-3cc2c(c1=O)COC(=O)[C@]2(O)CC1CC1]

LD-97f (7.1 mg, 24.2 μmol) and LD-109c (20.9 mg, 22.1 μmol) were dissolved in 0.2 mL of N,N-dimethylformamide, then added with 1-hydroxybenzotriazole (4.5 mg, 33.0 μmol), N,N-diisopropylethylamine (8.6 mg, 66.2 μmol) and 1-(3-dimethylaminopropyl)-3-ethylcarbodiimide hydrochloride (6.3 mg, 33.1 μmol) in sequence, and stirred at 25° C. for 2 hours. 5 mL of water was added to the reaction solution, dichloromethane (5 mL×3) was used for extraction, the organic phase was dried over anhydrous sodium sulfate and filtered, and the filtrate was concentrated by vacuum distillation. The residue was purified by pre-HPLC (chromatographic column: Phenomenex Kinetex EVO C18 150×30 mm×5 μm; mobile phase: A-water (0.225% formic acid), B-acetonitrile; gradient elution: B %: 37%-57%) to obtain the title product LD-111 (5.58 mg, yield: 20%) as a white solid.

MS m/z (ESI): 1183.6 [M+1].

¹H NMR (400 MHz, CD₃OD) δ 8.90-8.82 (m, 2H), 7.68-7.62 (m, 2H), 7.25-7.11 (m, 5H), 5.72-5.66 (m, 1H), 5.59-5.54 (m, 1H), 5.50-5.45 (m, 1H), 5.37-5.32 (m, 1H), 5.23-5.16 (m, 2H), 4.70-4.65 (m, 1H), 4.40-4.36 (m, 1H), 3.83-3.76 (m, 4H), 3.70-3.60 (m, 3H), 3.35-3.34 (m, 3H), 3.16-3.11 (m, 1H), 3.08-3.02 (m, 1H), 2.93-2.87 (m, 1H), 2.72-2.67 (m, 2H), 2.47-2.39 (m, 3H), 2.37-2.28 (m, 2H), 2.04-1.88 (m, 4H), 1.84-1.77 (m, 1H), 1.17-1.10 (m, 2H), 0.93-0.83 (m, 2H), 0.82-0.76 (m, 2H), 0.69-0.58 (m, 3H), 0.56-0.50 (m, 1H), 0.47-0.37 (m, 2H), 0.13-0.07 (m, 1H), 0.04-−0.01 (m, 1H).

### Example 3-112: LD-112

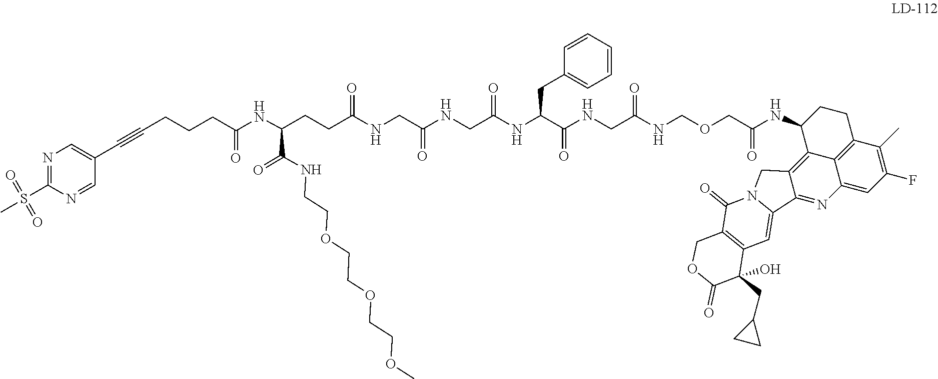 [SMILES: COCCOCCOCCNC(=O)[C@H](CCC(=O)NCC(=O)NCC(=O)N[C@@H](Cc1ccccc1)C(=O)NCC(=O)NCOCC(=O)N[C@H]1CCc2c(C)c(F)cc3nc4c(c1c23)Cn1c-4cc2c(c1=O)COC(=O)[C@]2(O)CC1CC1)NC(=O)CCCC#Cc1cnc(S(C)(=O)=O)nc1]

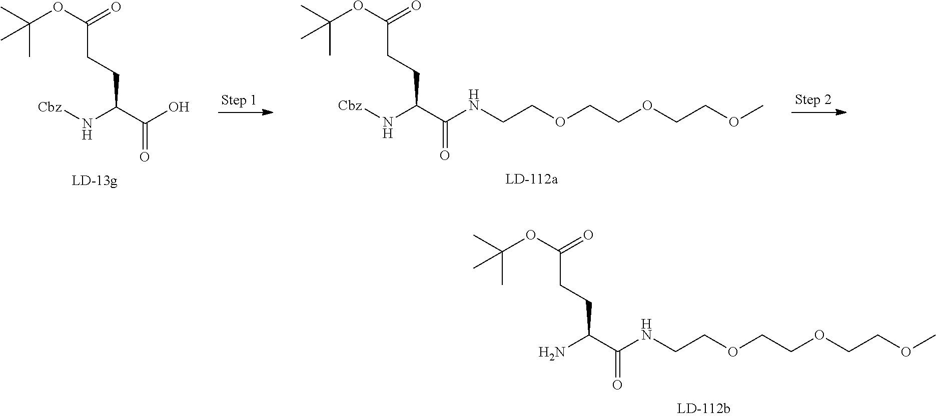 [SMILES: CC(C)(C)OC(=O)CC[C@H](NC(=O)OCc1ccccc1)C(=O)O ; COCCOCCOCCNC(=O)[C@H](CCC(=O)OC(C)(C)C)NC(=O)OCc1ccccc1 ; COCCOCCOCCNC(=O)[C@@H](N)CCC(=O)OC(C)(C)C]

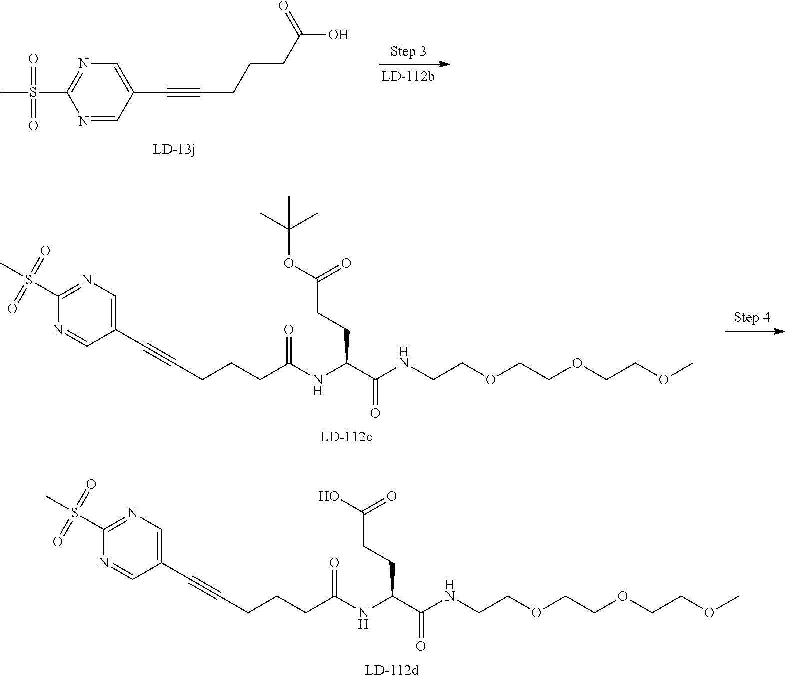

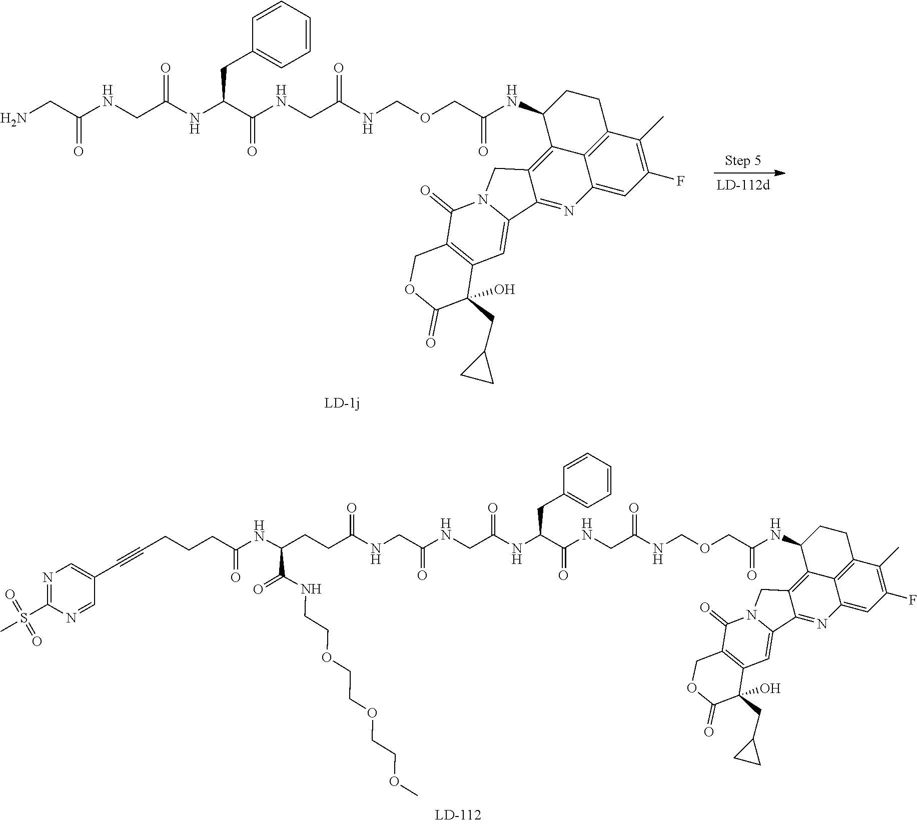 [SMILES: Cc1c(F)cc2nc3c(c4c2c1CC[C@@H]4NC(=O)COCNC(=O)CNC(=O)[C@H](Cc1ccccc1)NC(=O)CNC(=O)CN)Cn1c-3cc2c(c1=O)COC(=O)[C@]2(O)CC1CC1 ; COCCOCCOCCNC(=O)[C@H](CCC(=O)NCC(=O)NCC(=O)N[C@@H](Cc1ccccc1)C(=O)NCC(=O)NCOCC(=O)N[C@H]1CCc2c(C)c(F)cc3nc4c(c1c23)Cn1c-4cc2c(c1=O)COC(=O)[C@]2(O)CC1CC1)NC(=O)CCCC#Cc1cnc(S(C)(=O)=O)nc1]

### Step 1

LD-13 g (500 mg, 1.48 mmol) was dissolved in 5 mL of N,N-dimethylformamide, then added with 2-(7-azobenzotriazole)-N,N,N′,N′-tetramethyluronium hexafluorophosphate (564 mg, 1.48 mmol), N,N-diisopropylethylamine (574 mg, 4.44 mmol) and 3,6,9-trioxa-1-aminodecane (242 mg, 1.48 mmol) in sequence, and stirred at 25° C. for 2 hours. 15 mL of water was added, and ethyl acetate (30 mL×3) was used for extraction. The organic phase was dried over anhydrous sodium sulfate and filtered, and the filtrate was concentrated by distillation under reduced pressure. The resulting residue was purified by silica gel column chromatography with the eluent system A to obtain the title product LD-112a (600 mg, yield: 84%).

MS m/z (ESI): 483.3 [M+1].

### Step 2

LD-112a (600 mg, 1.24 mmol) was dissolved in 6 mL of methanol, added with palladium-on-carbon (60 mg, 10%, wetted with ca. 55% water) under nitrogen protection, subjected to replacement with hydrogen gas, and stirred at 25° C. for 1 hour. The reaction solution was subjected to replacement with hydrogen gas three times, and stirred at 25° C. for 1 hour under hydrogen gas (15 Psi). The reaction solution was filtered through Celite, and the filtrate was concentrated by vacuum distillation to obtain the title product LD-112b (350 mg, yield: 81%) as a colorless oil.

MS m/z (ESI): 349.3 [M+1].

### Step 3

LD-112b (350 mg, 1.01 mmol) was dissolved in 5 mL of N,N-dimethylformamide, then added with O-(7-azabenzotriazol-1-yl)-N,N,N′,N′-tetramethyluronium hexafluorophosphate (384 mg, 1.01 mmol), N,N-diisopropylethylamine (390 mg, 3.02 mmol) in sequence, then added with LD-13j (271 mg, 1.01 mmol), and stirred at 25° C. for 2 hours. 1 mL of water was added, and dichloromethane (3 mL×2) was used for extraction. The organic phase was dried over anhydrous sodium sulfate and filtered, and the filtrate was concentrated by vacuum distillation. The residue was purified by reverse phase high performance liquid chromatography (mobile phase: A-water, B-acetonitrile; isocratic elution: B %: 36%) to obtain the title product LD-112c (301.78 mg, yield: 50%) as a white solid.

MS m/z (ESI): 599.2 [M+1].

### Step 4

LD-112c (50.0 mg, 0.08 mmol) was dissolved in 1 mL of dichloromethane, then slowly added with 0.5 mL of trifluoroacetic acid, and stir at 25° C. for 0.5 hours. The reaction solution was concentrated by distillation to obtain the crude title product LD-112d (45 mg) as a colorless oil.

The product was used directly in the next reaction without purification.

MS m/z (ESI): 543.24 [M+1].

### Step 5

LD-112d (8.9 mg, 16 μmol) and formate of LD-1j (15.0 mg, 16.4 μmol) were in 1.5 mL of N,N-dimethylformamide, then added with 1-hydroxybenzotriazole (3.3 mg, 25 μmol), N,N-diisopropylethylamine (6.4 mg, 49 μmol) and 1-(3-dimethylaminopropyl)-3-ethylcarbodiimide hydrochloride (4.7 mg, 25 μmol) in sequence, and stirred at 25° C. for 1 hour. 50 mL of water was added to the reaction solution, and dichloromethane (45 mL×3) was used for extraction. The organic phase was dried over anhydrous sodium sulfate and filtered, and the filtrate was concentrated by vacuum distillation. The residue was purified by pre-HPLC (chromatographic column: Phenomenex luna C18 150×25 mm×10 μm; mobile phase: A-water (0.225% formic acid), B-acetonitrile; gradient elution: B %: 26%-56%) to obtain the title product LD-112 (7.80 mg, yield: 34%) as a white solid.

MS m/z (ESI): 1391.4 [M+1].

¹H NMR (400 MHz, CD₃OD) δ 8.97-8.87 (m, 2H), 7.68-7.58 (m, 2H), 7.29-7.13 (m, 5H), 5.76-5.69 (m, 1H), 5.61-5.53 (m, 1H), 5.38-5.27 (m, 2H), 5.16-5.06 (m, 1H), 4.84-4.79 (m, 2H), 4.68-4.63 (m, 1H), 4.40-4.32 (m, 2H), 4.27-4.21 (m, 1H), 4.18-4.13 (m, 1H), 3.94-3.81 (m, 4H), 3.69-3.64 (m, 2H), 3.62-3.59 (m, 1H), 3.59-3.58 (m, 3H), 3.54-3.47 (m, 4H), 3.43-3.36 (m, 2H), 3.36-3.34 (m, 3H), 3.33-3.32 (m, 2H), 3.28-3.25 (m, 2H), 3.21-3.17 (m, 1H), 3.00-2.96 (m, 1H), 2.95-2.90 (m, 1H), 2.58-2.53 (m, 2H), 2.50-2.31 (m, 9H), 2.28-2.23 (m, 1H), 2.11-2.04 (m, 1H), 1.96-1.85 (m, 4H), 1.81-1.74 (m, 1H), 0.89-0.82 (m, 1H), 0.44-0.35 (m, 2H), 0.10-0.04 (m, 1H), 0.01-−0.05 (m, 1H).

### Example 3-113: LD-113

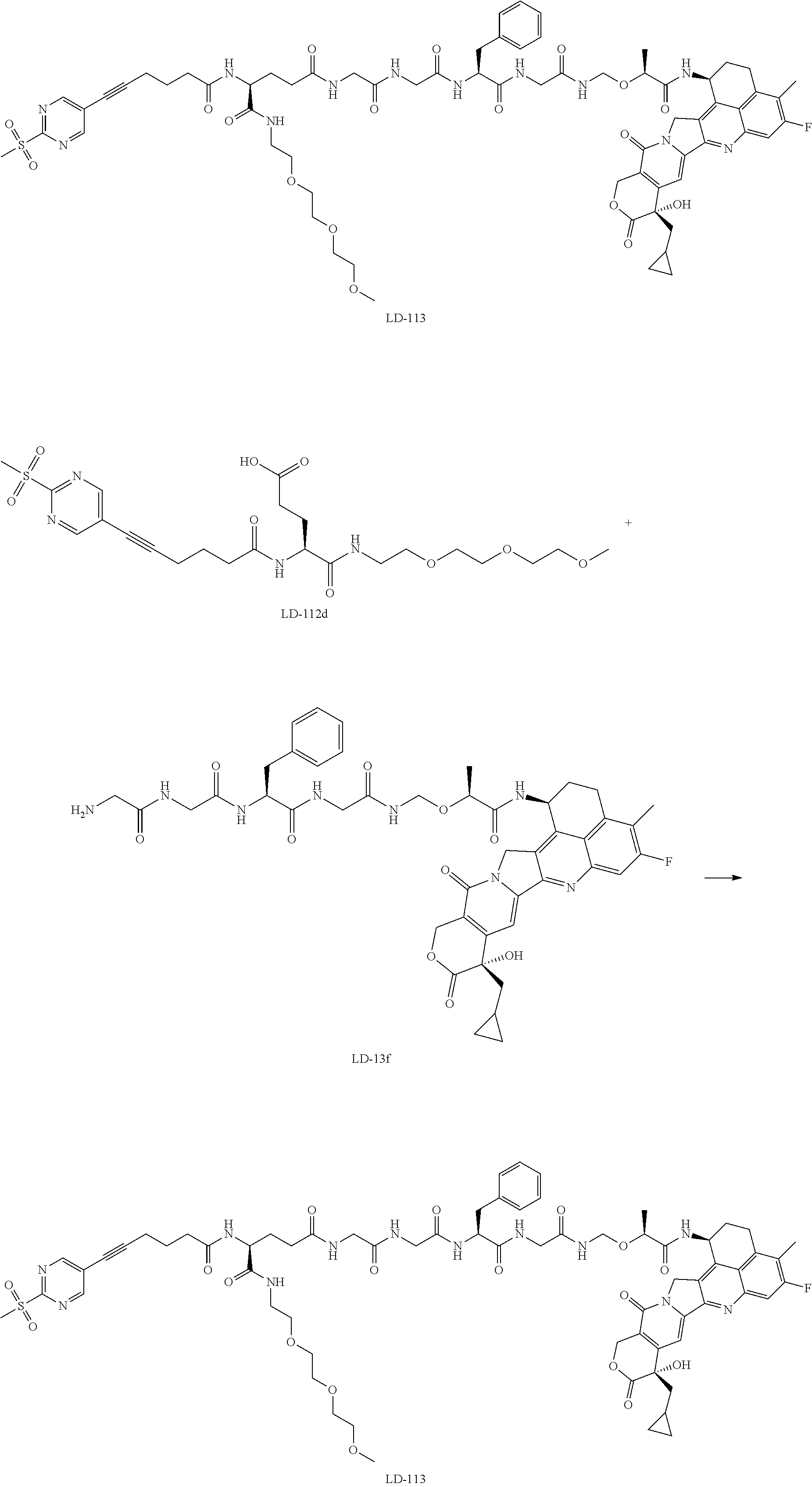

LD-112d (14.8 mg, 27.2 μmol), LD-13f (22.0 mg, 25.0 μmol) and O-(7-azabenzotriazol-1-yl)-N,N,N′N′-tetramethyluronium hexafluorophosphate (11.3 mg, 29.7 μmol) were dissolved in 1 mL of N,N-dimethylformamide, then added with N,N-diisopropylethylamine (9.6 mg, 74.2 μmol), and stirred at 25° C. for 1 hour. The reaction solution was concentrated by vacuum distillation and the residue was purified by Pre-HPLC purification (chromatographic column: InfinityLab Poroshell 120 SB-C18 21.2×150 mm, 4 μm; mobile phase: A-water (0.1% formic acid), B-acetonitrile; gradient elution: B %: 10%-90%) to obtain the title product LD-113 (19.45 mg, yield: 55%) as a white solid.

MS m/z (ESI): 1405.9 [M+1].

¹H NMR (400 MHz, DMSO-d6) δ 9.14-9.08 (m, 2H), 8.70-8.64 (m, 1H), 8.61-8.54 (m, 1H), 8.37-8.29 (m, 1H), 8.14-7.95 (m, 5H), 7.80-7.75 (m, 1H), 7.40-7.36 (m, 1H), 7.27-7.13 (m, 5H), 6.63-6.57 (m, 1H), 5.58-5.50 (m, 1H), 5.47-5.35 (m, 2H), 5.31-5.15 (m, 2H), 4.77-4.68 (m, 1H), 4.62-4.54 (m, 1H), 4.51-4.41 (m, 1H), 4.26-4.18 (m, 1H), 4.16-4.08 (m, 1H), 3.77-3.70 (m, 1H)), 3.69-3.64 (m, 3H), 3.62-3.54 (m, 1H), 3.51-3.45 (m, 6H), 3.42-3.36 (m, 7H), 3.34-3.34 (m, 2H), 3.26-3.09 (m, 7H)), 3.04-2.95 (m, 1H), 2.79-2.69 (m, 1H), 2.57-2.51 (m, 2H), 2.41-2.34 (m, 3H), 2.34-2.26 (m, 2H), 2.18-2.11 (m, 3H), 1.90-1.66 (m, 6H), 1.35-1.27 (m, 3H), 0.86-0.75 (m, 1H), 0.40-0.25 (m, 2H), 0.09-0.02 (m, 1H), −0.04-−0.13 (m, 1H).

### Example 3-114: LD-114

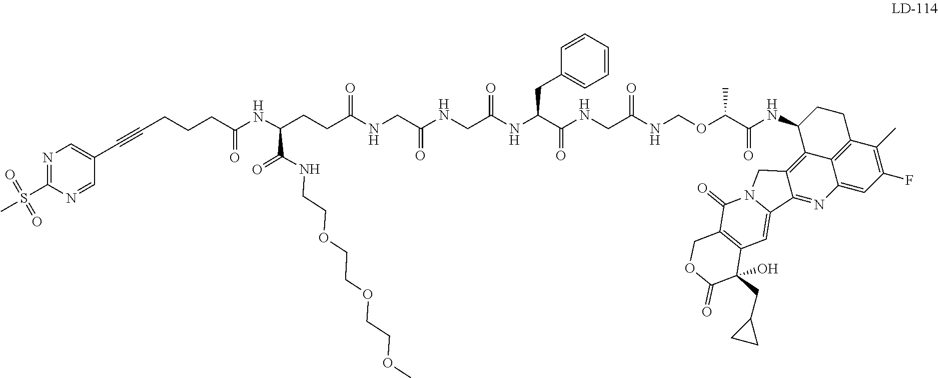 [SMILES: COCCOCCOCCNC(=O)[C@H](CCC(=O)NCC(=O)NCC(=O)N[C@@H](Cc1ccccc1)C(=O)NCC(=O)NCO[C@H](C)C(=O)N[C@H]1CCc2c(C)c(F)cc3nc4c(c1c23)Cn1c-4cc2c(c1=O)COC(=O)[C@]2(O)CC1CC1)NC(=O)CCCC#Cc1cnc(S(C)(=O)=O)nc1]

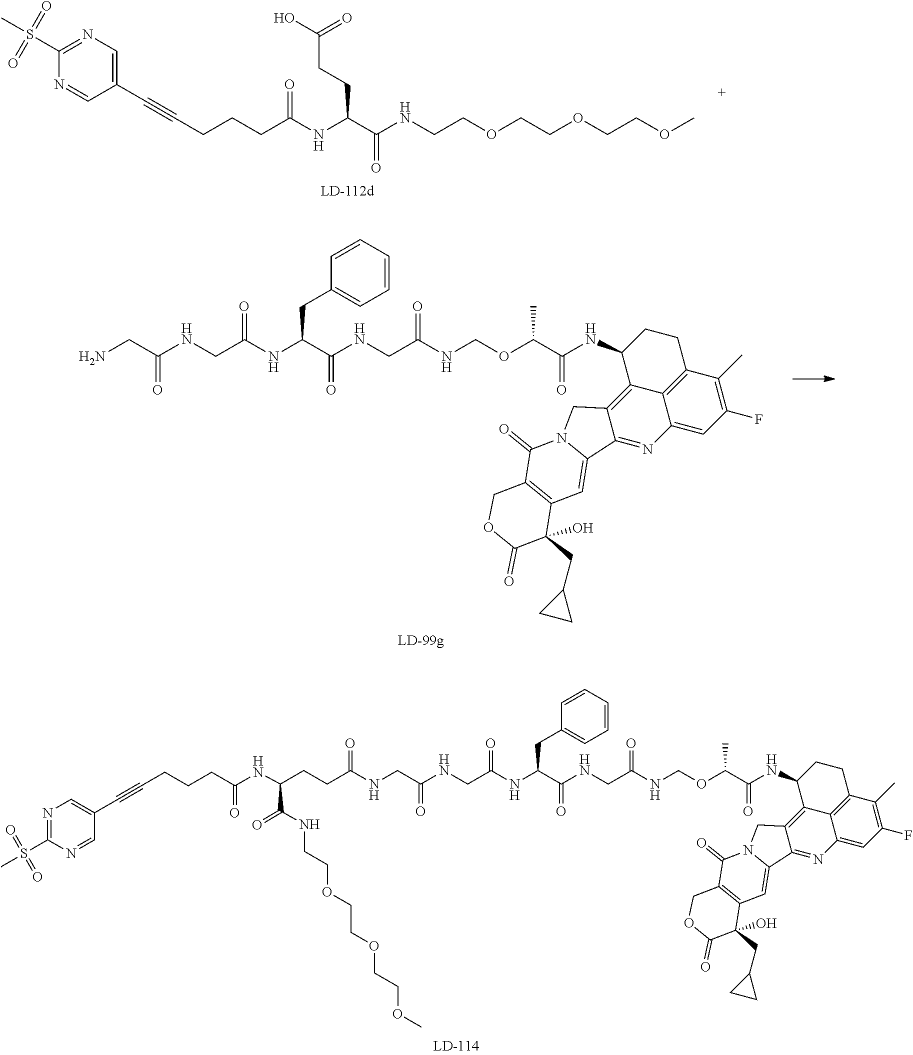

LD-112d (10.2 mg, 18.7 mol) and LD-99 g (15.0 mg, 17.0 mol) were dissolved in 1.0 mL of N,N-dimethylformamide, then added with 1-hydroxybenzotriazole (3.5 mg, 25.5 mol), N,N-diisopropylethylamine (6.6 mg, 51-mol) and 1-(3-dimethylaminopropyl)-3-ethylcarbodiimide hydrochloride (4.9 mg, 25.5 mol), and stirred at 25 HC for 2 hours. 3 mL of water was added to the reaction solution, dichloromethane (5 mL×2) was used for extraction, the organic phase was dried over anhydrous sodium sulfate and filtered, and the filtrate was concentrated by vacuum distillation. The obtained residue was purified by pre-HPLC (chromatographic column: Phenomenex luna C18 150×25 mm×10 μm; mobile phase: A-water (0.225% formic acid), B-acetonitrile; gradient elution: B %: 28%-58%) to obtain the title product LD-114 (10.1 mg, yield: 42%) as a white solid.

MS m/z (ESI): 1405.7 [M+1].

¹H NMR (400 MHz, CD₃OD) δ 8.95-8.88 (m, 2H), 7.68-7.61 (m, 2H), 7.29-7.21 (m, 2H), 7.20-7.12 (m, 3H), 5.68-5.64 (m, 1H), 5.60-5.54 (m, 1H), 5.42-5.33 (m, 2H), 5.16-5.10 (m, 1H), 4.82-4.79 (m, 1H), 4.70-4.66 (m, 1H), 4.43-4.35 (m, 2H), 4.26-4.20 (m, 1H), 3.90-3.80 (m, 4H), 3.78-3.73 (m, 1H), 3.71-3.66 (m, 1H), 3.63-3.54 (m, 6H), 3.54-3.46 (m, 5H), 3.43-3.35 (m, 4H), 3.35-3.33 (m, 3H), 3.27-3.22 (m, 1H), 3.21-3.16 (m, 1H), 3.15-3.12 (m, 1H), 3.07-3.02 (m, 1H), 2.92-2.86 (m, 1H), 2.59-2.52 (m, 2H), 2.47-2.38 (m, 5H), 2.36-2.29 (m, 3H), 2.11-2.04 (m, 1H), 1.99-1.87 (m, 4H), 1.85-1.79 (m, 1H), 1.60-1.51 (m, 3H), 0.92-0.85 (m, 1H), 0.49-0.39 (m, 2H), 0.15-0.08 (m, 1H), 0.06-0.00 (m, 1H).

### Example 3-115: LD-115

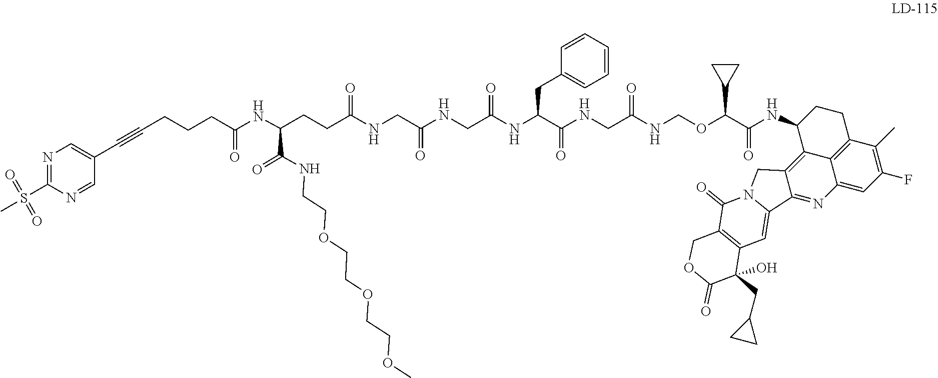 [SMILES: COCCOCCOCCNC(=O)[C@H](CCC(=O)NCC(=O)NCC(=O)N[C@@H](Cc1ccccc1)C(=O)NCC(=O)NCO[C@H](C(=O)N[C@H]1CCc2c(C)c(F)cc3nc4c(c1c23)Cn1c-4cc2c(c1=O)COC(=O)[C@]2(O)CC1CC1)C1CC1)NC(=O)CCCC#Cc1cnc(S(C)(=O)=O)nc1]

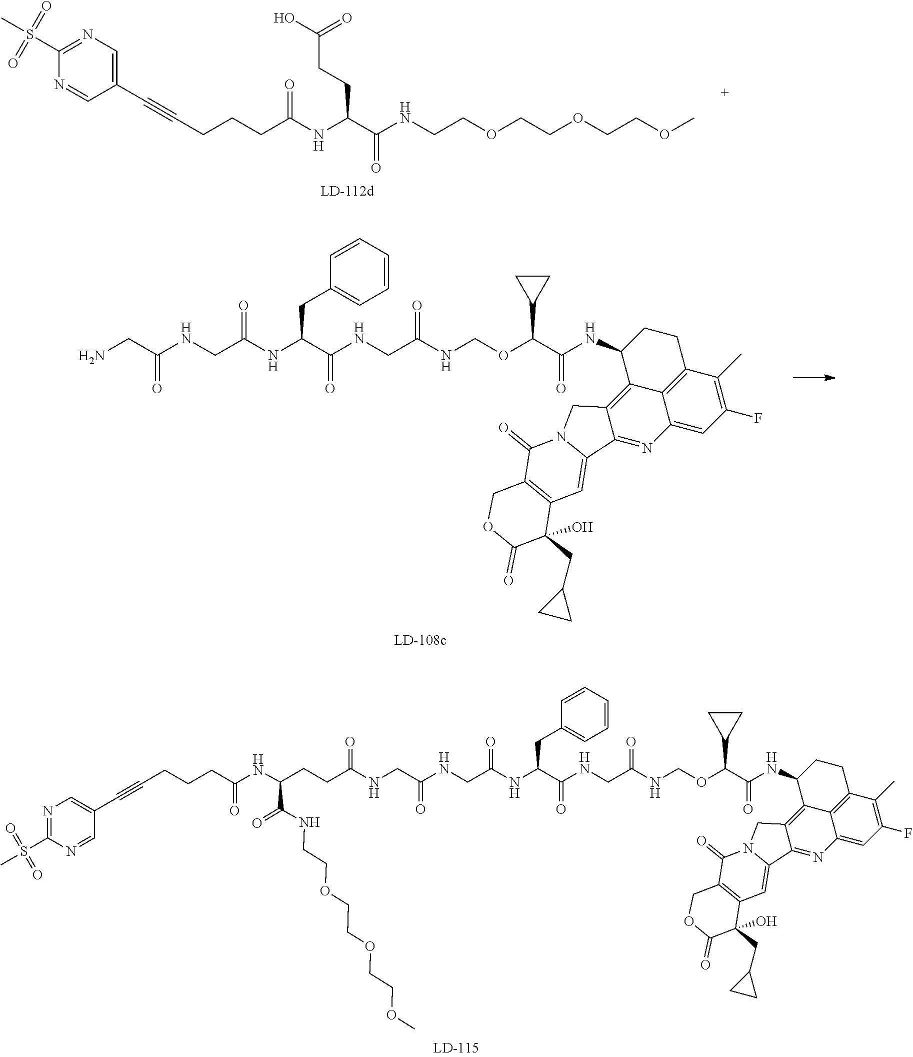

LD12d(8.4 mg, 16 μmol) and formate of LD-108c (15.0 mg, 15.5 μmol) were dissolved in 1.5 mL of N,N-dimethylformamide, then added with 1-hydroxybenzotriazole (3.2 mg, 23 μmol), N,N-diisopropylethylamine (6.0 mg, 47 μmol) and 1-(3-dimethylaminopropyl)-3-ethylcarbodiimide hydrochloride (4.5 mg, 23 μmol) in sequence, and stirred at 25° C. for 3 hours. 50 mL of water was added to the reaction solution, dichloromethane (45 mL×3) was used for extraction. The organic phase was dried over anhydrous sodium sulfate and filtered, and the filtrate was concentrated by vacuum distillation. The residue was purified by pre-HPLC (chromatographic column: Phenomenex luna C18 150×25 mm×10 μm; mobile phase: A-water (0.225% formic acid), B-acetonitrile; gradient elution: B %: 30%-60%) to obtain the title product LD-115 (8.37 mg, yield: 37%) as a white solid.

MS m/z (ESI): 1431.2 [M+1].

¹H NMR (400 MHz, CD₃OD) δ 8.96-8.89 (m, 2H), 7.65-7.57 (m, 2H), 7.26-7.13 (m, 5H), 5.70-5.64 (m, 1H), 5.60-5.54 (m, 1H), 5.49-5.41 (m, 1H), 5.34-5.28 (m, 1H), 5.14-5.07 (m, 1H), 4.77-4.72 (m, 1H), 4.41-4.33 (m, 2H), 3.92-3.86 (m, 3H), 3.80-3.75 (m, 2H), 3.69-3.65 (m, 2H), 3.62-3.57 (m, 6H), 3.56-3.47 (m, 5H), 3.44-3.37 (m, 2H), 3.36-3.34 (m, 3H), 3.33-3.33 (m, 2H), 3.29-3.23 (m, 2H), 3.21-3.11 (m, 2H), 3.02-2.96 (m, 1H), 2.92-2.84 (m, 1H), 2.58-2.54 (m, 2H), 2.45-2.40 (m, 2H), 2.39-2.33 (m, 5H), 2.23-2.17 (m, 1H), 2.11-2.02 (m, 1H), 1.97-1.84 (m, 4H), 1.79-1.73 (m, 1H), 1.24-1.16 (m, 1H), 0.89-0.81 (m, 1H), 0.66-0.52 (m, 4H), 0.45-0.34 (m, 2H), 0.11-0.04 (m, 1H), 0.01-−0.06 (m, 1H).

### Example 3-116: LD-116

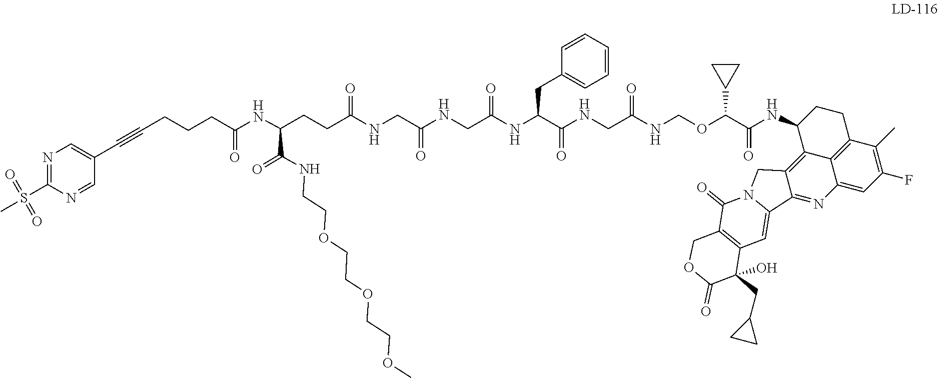 [SMILES: COCCOCCOCCNC(=O)[C@H](CCC(=O)NCC(=O)NCC(=O)N[C@@H](Cc1ccccc1)C(=O)NCC(=O)NCO[C@@H](C(=O)N[C@H]1CCc2c(C)c(F)cc3nc4c(c1c23)Cn1c-4cc2c(c1=O)COC(=O)[C@]2(O)CC1CC1)C1CC1)NC(=O)CCCC#Cc1cnc(S(C)(=O)=O)nc1]

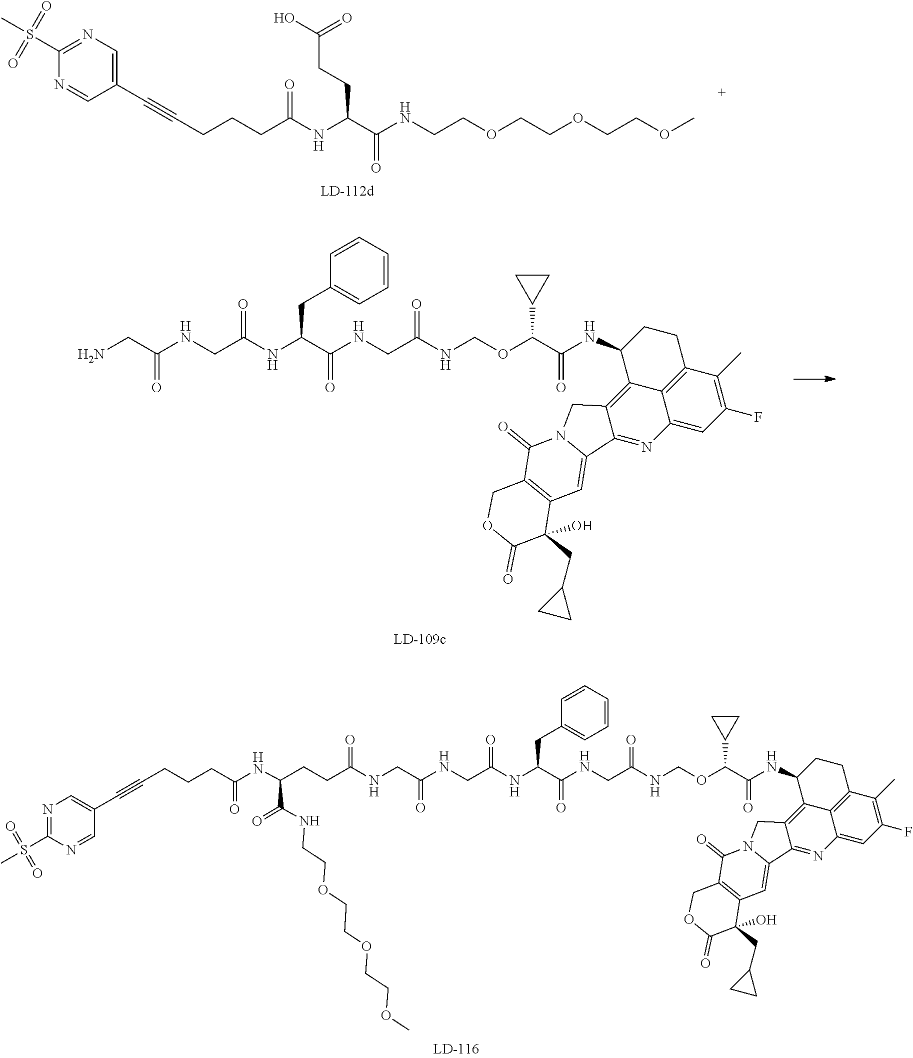

LD-112d (9.17 mg, 16.9 μmol) and formate of LD-109c (15.0 mg, 15.3 μmol) were dissolved in 1.5 mL of N,N-dimethylformamide, then added with 1-hydroxybenzotriazole (3.11 mg, 23.0 μmol), N,N-diisopropylethylamine (5.96 mg, 46.0 μmol) and 1-(3-dimethylaminopropyl)-3-ethylcarbodiimide hydrochloride (4.42 mg, 23.0 μmol) in sequence, and stirred at 25° C. for 2 hours. 3 mL of water was added to the reaction solution, dichloromethane (5 mL×2) was used for extraction, the organic phase was dried over anhydrous sodium sulfate and filtered, and the filtrate was concentrated by vacuum distillation. The obtained residue was purified by pre-HPLC (chromatographic column: Phenomenex luna C18 150×25 mm×10 μm; mobile phase: A-water (0.225% formic acid), B-acetonitrile; gradient elution: B %: 27%-57%) to obtain the title product LD-116 (8.75 mg, yield: 39%) as a white solid.

MS m/z (ESI): 1431.7 [M+1].

¹H NMR (400 MHz, CD₃OD) δ 8.94-8.89 (m, 2H), 7.65-7.55 (m, 2H), 7.26-7.20 (m, 2H), 7.19-7.10 (m, 3H), 5.73-5.65 (m, 1H), 5.60-5.52 (m, 1H), 5.45-5.31 (m, 2H), 5.13-5.05 (m, 1H), 4.68-4.62 (m, 1H), 4.42-4.34 (m, 2H), 3.90-3.83 (m, 3H), 3.81-3.75 (m, 2H), 3.74-3.45 (m, 14H), 3.43-3.34 (m, 5H), 3.32-3.32 (m, 2H), 3.29-3.25 (m, 2H), 3.22-3.16 (m, 1H), 3.07-3.00 (m, 1H), 2.92-2.85 (m, 1H), 2.60-2.52 (m, 2H), 2.44-2.42 (m, 1H), 2.41-2.39 (m, 3H), 2.38-2.32 (m, 3H), 2.31-2.26 (m, 1H), 2.12-2.03 (m, 1H), 1.97-1.87 (m, 4H), 1.84-1.76 (m, 1H), 1.33-1.27 (m, 1H), 0.92-0.84 (m, 1H), 0.70-0.58 (m, 3H), 0.56-0.49 (m, 1H), 0.47-0.37 (m, 2H), 0.14-0.06 (m, 1H), 0.05-−0.03 (m, 1H).

### IV. ADC Conjugate Examples

### Steps for Preparation of Antibody Conjugates:

A: An antibody was exchanged into 50 mM PBS/1.0 mM EDTA (ethylenediaminetetraacetic acid) buffer (adjusted with sodium hydroxide solution to pH 6.0) using an ultrafiltration tube with a molecular weight cutoff of 50 kD, 5 to 10 equivalents of 10 mM TCEP (tris(2-carboxyethyl)phosphine) aqueous solution was added, and shaken at 37° C. for 3 hours. A linker-drug conjugate was dissolved in DMSO, 10 to 20 equivalents of the linker-drug conjugate was taken therefrom and added dropwise to the reduced antibody solution, mixed well by vortexing and shaking, and the shaking was continued at 25° C. for 2 hours. After the reaction was completed, 200 equivalents of 100 mM NAC (N-acetylcysteine) aqueous solution was added, shaken at 25° C. for 20 minutes to terminate the linking reaction, an ultrafiltration tube with a molecular weight cutoff of 50 kD or a Sephadex G-25 desalting column was used to remove excess small molecules, and the antibody-drug conjugate was exchanged into 10 mM PBS buffer (pH 6.0). After ultrafiltration purification, the sample was filtered with a 0.22 μm filter membrane to obtain the unhydrolyzed antibody-drug conjugate, which was stored in a refrigerator at 4° C. A part of the unhydrolyzed antibody-drug conjugate was taken, and exchanged into 50 mM PBS/1.0 mM EDTA buffer (pH 7.8) using an ultrafiltration tube with a molecular weight cutoff of 50 kD, and shaken at a constant temperature of 35° C. for 2 hours, the antibody-drug conjugate was exchanged into 10 mM PBS buffer (pH 6.0) using an ultrafiltration tube with a molecular weight cutoff of 50 kD. After ultrafiltration purification, the sample was filtered with a 0.22 μm filter membrane to obtain the hydrolyzed antibody-drug conjugate, which was stored in a refrigerator at 4° C. The DAR value of the conjugate was determined by reversed-phase high-performance liquid chromatography or mass spectrometry. A-1: An antibody was exchanged into 50 mM PBS/1.0 mM EDTA buffer (adjusted with sodium hydroxide solution to pH 6.5) using an ultrafiltration tube with a molecular weight cutoff of 50 kD, 5 to 10 equivalents of 10 mM TCEP aqueous solution was added and shaken at 25° C. for 3 hours. A linker-drug conjugate was dissolved in DMSO, 10 to 20 equivalents of the linker-drug conjugate was taken therefrom, added dropwise to the reduced antibody solution, mixed well by vortexing and shaking, and the shaking was continued at 25° C. for 2 hours; after the reaction was completed, 40 equivalents of 100 mM NAC aqueous solution was added and shaken at 25° C. for 20 minutes to terminate the linking reaction, and an ultrafiltration tube with a molecular weight cutoff of 50 kD or a Sephadex G-25 desalting column was used to remove excess small molecules, the antibody-drug conjugate was exchanged into 50 mM PBS buffer (pH 6.0). After ultrafiltration purification, the sample was filtered with a 0.22 μm filter membrane to obtain the antibody-drug conjugate, which was stored in a refrigerator at 4° C. A part of the unhydrolyzed antibody-drug conjugate was taken and exchanged into 50 mM PBS/1.0 mM EDTA buffer (pH 7.6) using an ultrafiltration tube with a molecular weight cutoff of 50 kD, and shaken at a constant temperature of 35° C. for 2 hours. The antibody-drug conjugate was exchanged into 50 mM PBS buffer (pH 6.0) using an ultrafiltration tube with a molecular weight cutoff of 50 kD. After ultrafiltration purification, the sample was filtered with a 0.22 μm filter membrane to obtain the hydrolyzed antibody-drug conjugate, which was stored in a refrigerator at 4° C. The DAR value of the conjugate was determined by reversed-phase high-performance liquid chromatography or mass spectrometry.
B: An antibody was exchanged into 50 mM PBS/1.0 mM EDTA buffer (adjusted with sodium hydroxide solution to pH 6.5) using an ultrafiltration tube with a molecular weight cutoff of 50 kD, 5 to 10 equivalents of 10 mM TCEP aqueous solution was added, and shaken at 25° C. for 3 hours. A linker-drug conjugate was dissolved in DMSO, 10 to 20 equivalents of the linker-drug conjugate was taken therefrom, added dropwise to the reduced antibody solution, and mixed well by vortexing and shaking, and the shaking was continued at 25° C. for 2 hours; after the reaction was completed, 40 equivalents of 100 mM NAC aqueous solution was added and shaken at 25° C. for 20 minutes to terminate the linking reaction, an ultrafiltration tube with a molecular weight cutoff of 50 kD or a Sephadex G-25 desalting column was used to remove excess small molecules, and the antibody-drug conjugate was exchanged into 50 mM PBS buffer (pH 6.0). After ultrafiltration purification, the sample was filtered with a 0.22 μm filter membrane to obtain the antibody-drug conjugate, which was stored in a refrigerator at 4° C. The DAR value of the conjugate was determined by reversed-phase high-performance liquid chromatography or mass spectrometry.
C: An antibody was exchanged into 50 mM PBS/1.0 mM EDTA buffer (adjusted with sodium hydroxide solution to pH 7.4) using an ultrafiltration tube with a molecular weight cutoff of 50 kD, 5 to 10 equivalents of 10 mM TCEP aqueous solution was added and shaken at 25° C. for 3 hours. A linker-drug conjugate was dissolved in DMSO, 10 to 20 equivalents of the linker-drug conjugate was taken therefrom, added to the reduced antibody solution, and mixed well by vortexing and shaking, and the shaking was continued at 25° C. for 2 hours; after the reaction was completed, 40 equivalents of 100 mM NAC aqueous solution was added and shaken at 25° C. for 20 minutes to terminate the linking reaction, an ultrafiltration centrifuge tube with a molecular weight cutoff of 50 kD or a Sephadex G-25 desalting column was used to remove excess small molecules, and the antibody-drug conjugate was exchanged into 50 mM PBS buffer (pH 6.0), and the sample was filtered with a 0.22 μm filter membrane to obtain the antibody-drug conjugate, which was stored in a refrigerator at 4° C. The DAR value of the conjugate was determined by reverse phase high performance liquid chromatography or mass spectrometry.

### Example 4-1: ADC-1

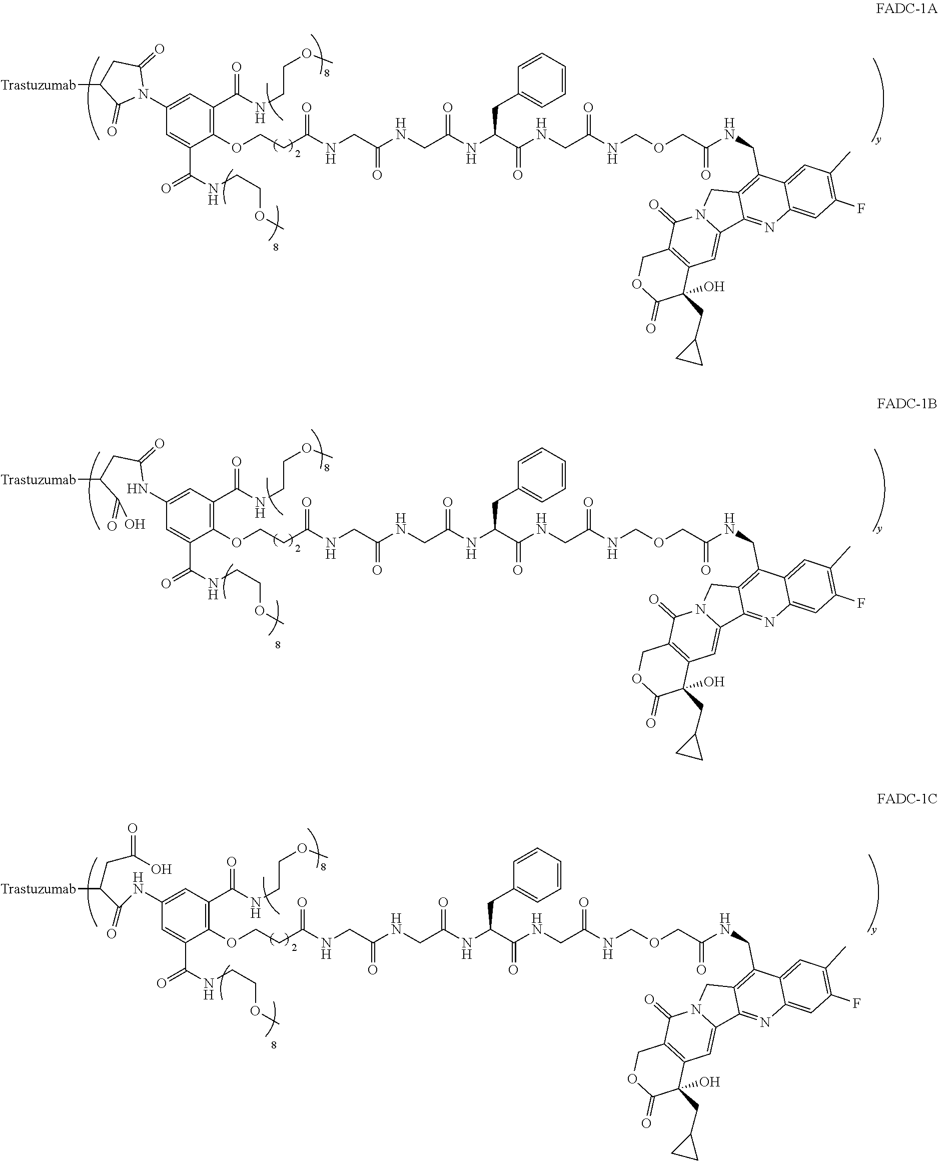

Using antibody Trastuzumab and LD-1 as starting materials, the preparation was carried out by applying step A. The PBS buffer with the exemplary product ADC-1 of the conjugate mixture (mixture of FADC-1A and/or FADC-1B and/or FADC-1C) was obtained and stored at 4° C.

### Example 4-2: ADC-2 to Example 4-10: ADC-10

| | |
|---|---|
| 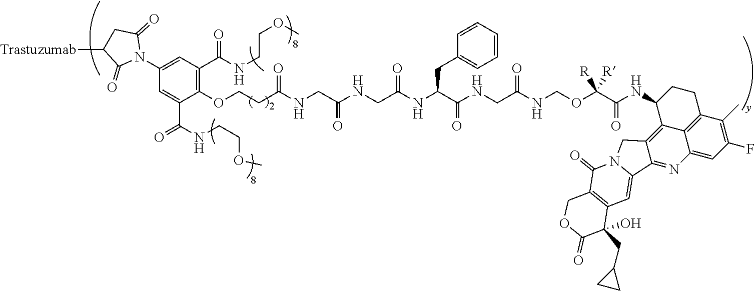 | FADC-X₁A |
| 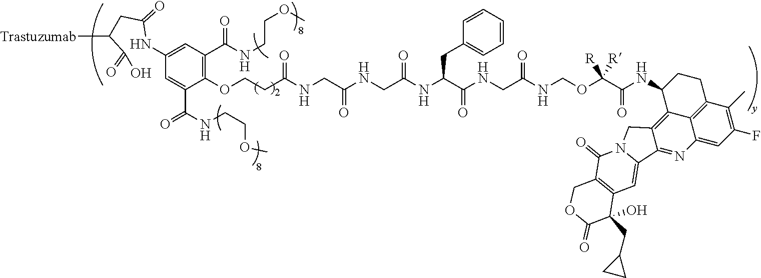 | FADC-X₁B |
| 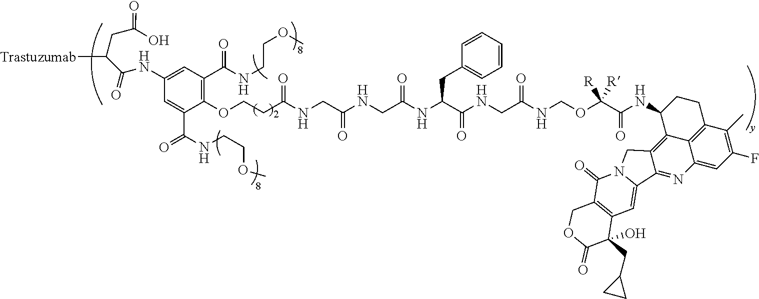 | FADC-X₁C |

| Exemplary ADC | Mixture of conjugates FADC-X₁A | | |
|---|---|---|---|
| product | and/or FADC-X₁B and/or FADC-X₁C | R | R′ |
| ADC-2 | FADC-2A and/or FADC-2B and/or | —CH₃ | —H |
| | FADC-2C | | |
| ADC-3 | FADC-3A and/or FADC-3B and/or | —H | —CH₃ |
| | FADC-3C | | |
| ADC-4 | FADC-4A and/or FADC-4B and/or FADC-4C | 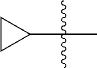 [SMILES: *C1CC1] | —H |
| ADC-5 | FADC-5A and/or FADC-5B and/or FADC-5C | —H | 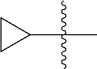 [SMILES: *C1CC1] |

| | |
|---|---|
| 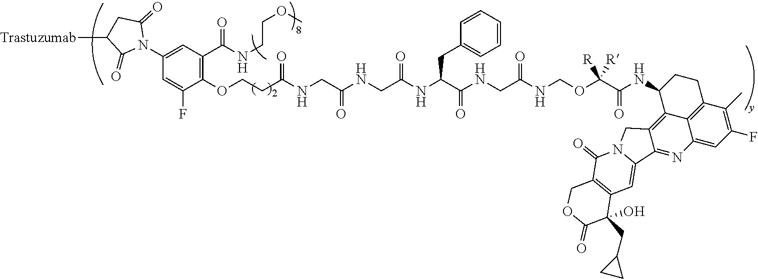 | FADC-X₂A |
| 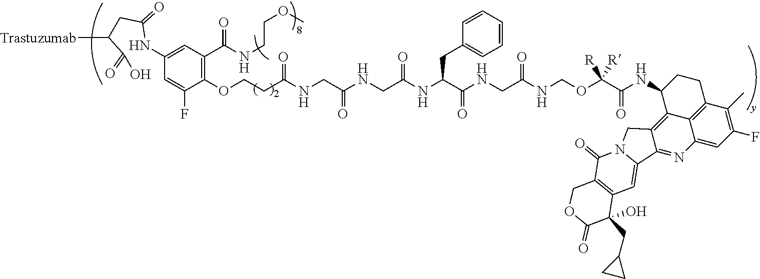 | FADC-X₂B |
| 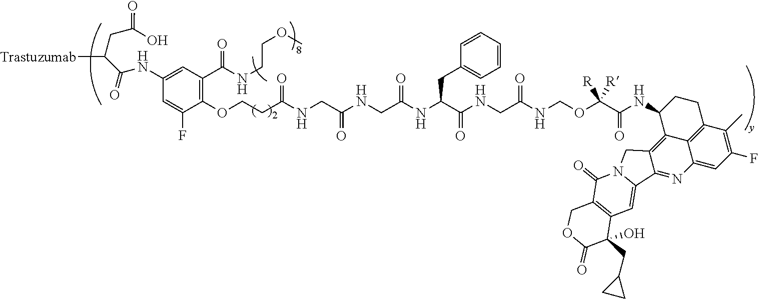 | FADC-X₂C |

| | Mixture of conjugates FADC-X₂A | | |
|---|---|---|---|
| Exemplary ADC | and/or FADC-X₂B and/or FADC- | | |
| product | X₂C | R | R′ |
| ADC-6 | FADC-6A and/or FADC-6B and/or | —H | —H |
| | FADC-6C | | |
| ADC-7 | FADC-7A and/or FADC-7B and/or | —CH₃ | —H |
| | FADC-7C | | |
| ADC-8 | FADC-8A and/or FADC-8B and/or | —H | —CH₃ |
| | FADC-8C | | |
| ADC-9 | FADC-9A and/or FADC-9B and/or FADC-9C | 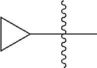 [SMILES: *C1CC1] | —H |
| ADC-10 | FADC-10A and/or FADC-10B and/or FADC-10C | —H | 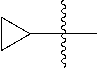 [SMILES: *C1CC1] |

LD-2, LD-3, LD-4, LD-5, LD-6, LD-7, LD-8, LD-9 and LD-10 were used to replace LD-1 respectively, and a similar preparation method was applied to obtain the corresponding ADC-2, ADC-3, ADC-4, ADC-5, ADC-6, ADC-7, ADC-8, ADC-9 and ADC-10, which were Examples 4-2 to 4-10.

### Example 4-11: ADC-11

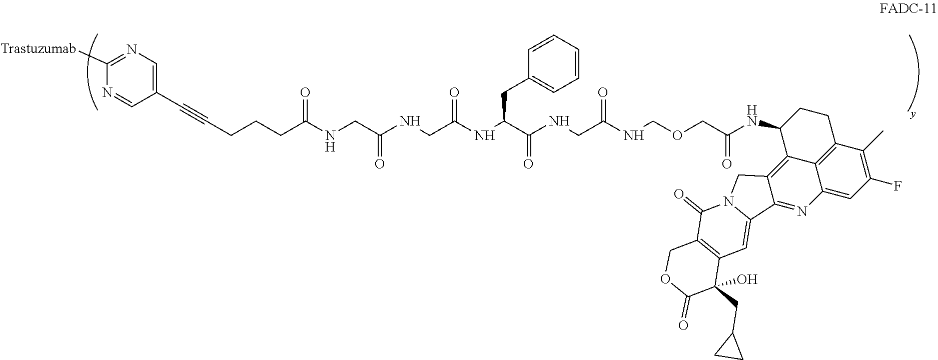

Using the antibody Trastuzumab and LD-11 as starting materials, the preparation was carried out by applying the method disclosed in the patent application “WO2019114666A1”. The PBS buffer with the exemplary product ADC-11 of the general Formula FADC-11 was obtained and stored at 4° C.

### Example 4-12: ADC-12 to Example 4-16: ADC-16

| | |
|---|---|
| 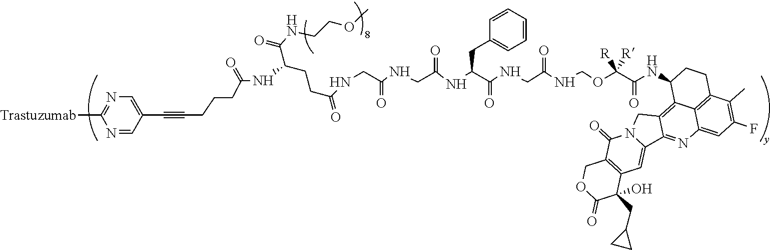 | FADC-X₃ |

| | General formula FADC-X₃ | |
|---|---|---|
| Exemplary ADC product | R | R′ |
| ADC-12 | —H | —H |
| ADC-13 | —CH₃ | —H |
| ADC-14 | —H | —CH₃ |
| ADC-15 | 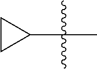 [SMILES: *C1CC1] | —H |
| ADC-16 | —H | 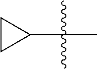 [SMILES: *C1CC1] |

Using the antibody Trastuzumab and LD-13 as starting materials, the preparation was carried out by applying the method of step C. The PBS buffer with the exemplary product ADC-13 of the conjugate mixture FADC-13 was obtained and stored at 4° C.

The average drug loading was calculated by mass spectrometry: y=7.36.

LD-12, LD-14, LD-15 and LD-16 were used to replace LD-13, respectively, and similar preparation methods were used to obtain the corresponding ADC-12, ADC-14, LD-15 and ADC-16, which were Example 4-12, and Examples 4-14 to 4-16.

### Example 4-17: ADC-17

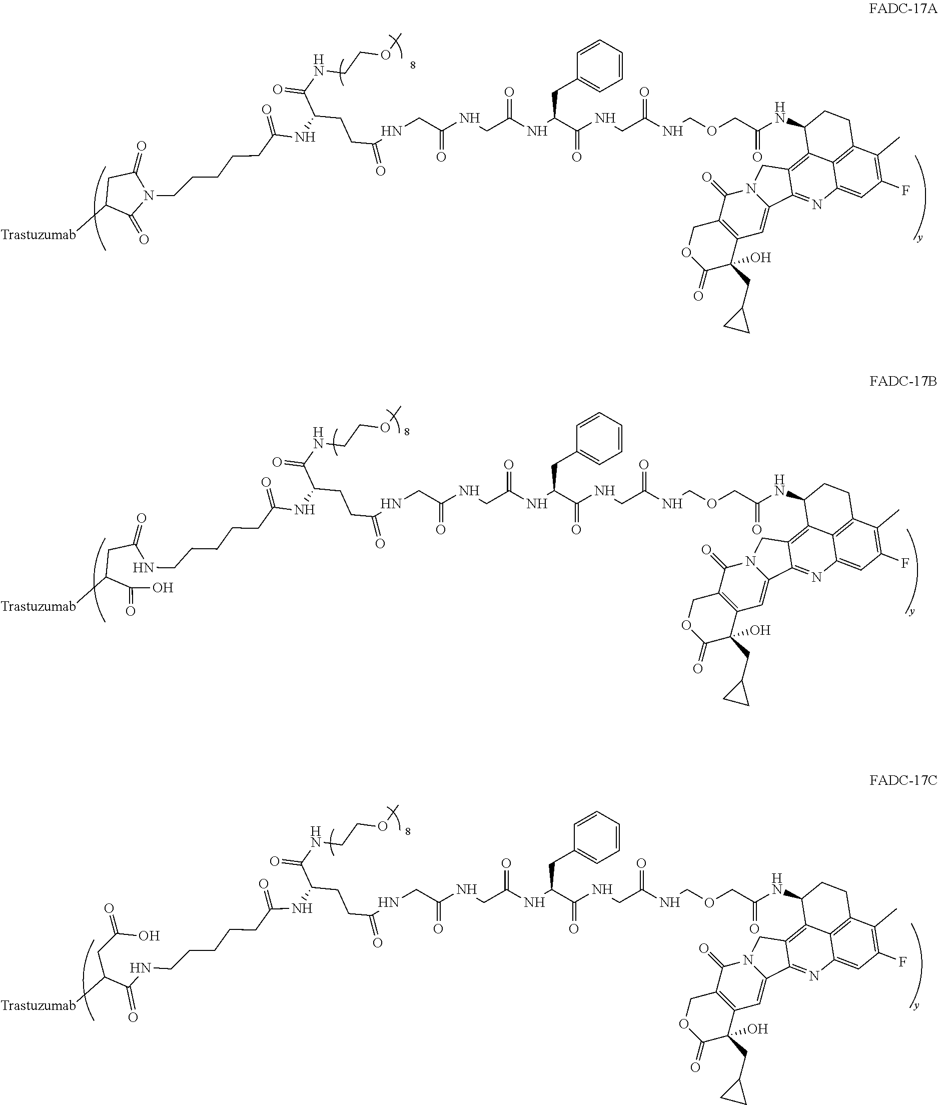

Using antibody Trastuzumab and LD-17 as starting materials, the preparation was carried out by applying the method disclosed in patent application “WO2022068898A1”. The PBS buffer with the exemplary product ADC-17 of the conjugate mixture (mixture of FADC-17A and/or FADC-17B and/or FADC-17C) was obtained and stored at 4° C.

### Example 4-18: ADC-18 to Example 4-21: ADC-21

| | |
|---|---|
| 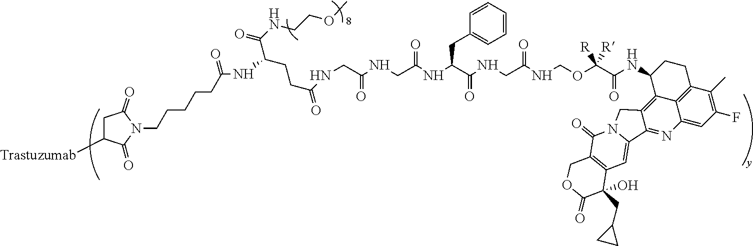 | FADC-X₄A |
| 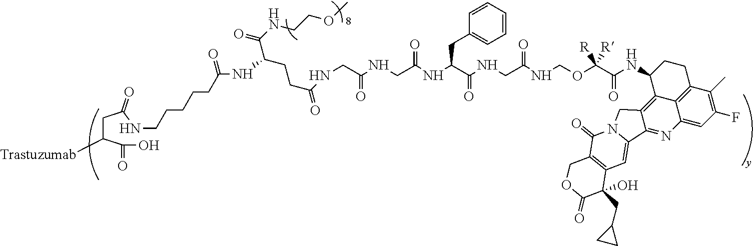 | FADC-X₄B |
| 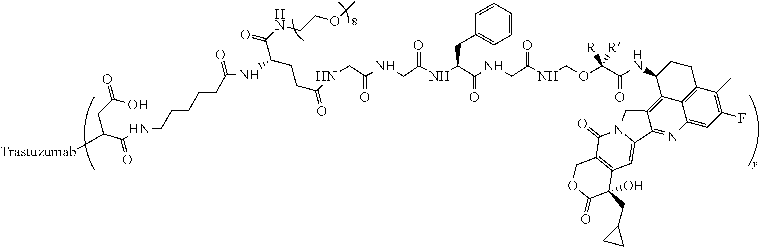 | FADC-X₄C |

| | Conjugate mixture of FADC-X₄A | | |
|---|---|---|---|
| Exemplary ADC | and/or FADC-X₄B and/or FADC- | | |
| product | X₄C | R | R′ |
| ADC-18 | FADC-18A and/or FADC-18B | —CH₃ | —H |
| | and/or FADC-18C | | |
| ADC-19 | FADC-19A and/or FADC-19B | —H | —CH₃ |
| | and/or FADC-19C | | |
| ADC-20 | FADC-20A and/or FADC-20B and/or FADC-20C | 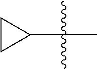 [SMILES: *C1CC1] | —H |
| ADC-21 | FADC-21A and/or FADC-21B and/or FADC-21C | —H | 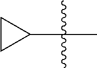 [SMILES: *C1CC1] |

LD-18, LD-19, LD-20 and LD-21 were used to replace LD-17, respectively, and the corresponding ADC-18, ADC-19, ADC-20 and ADC-21 were obtained by applying similar preparation methods, which were Examples 4-18 to 4-21.

### Example 4-22: ADC-22

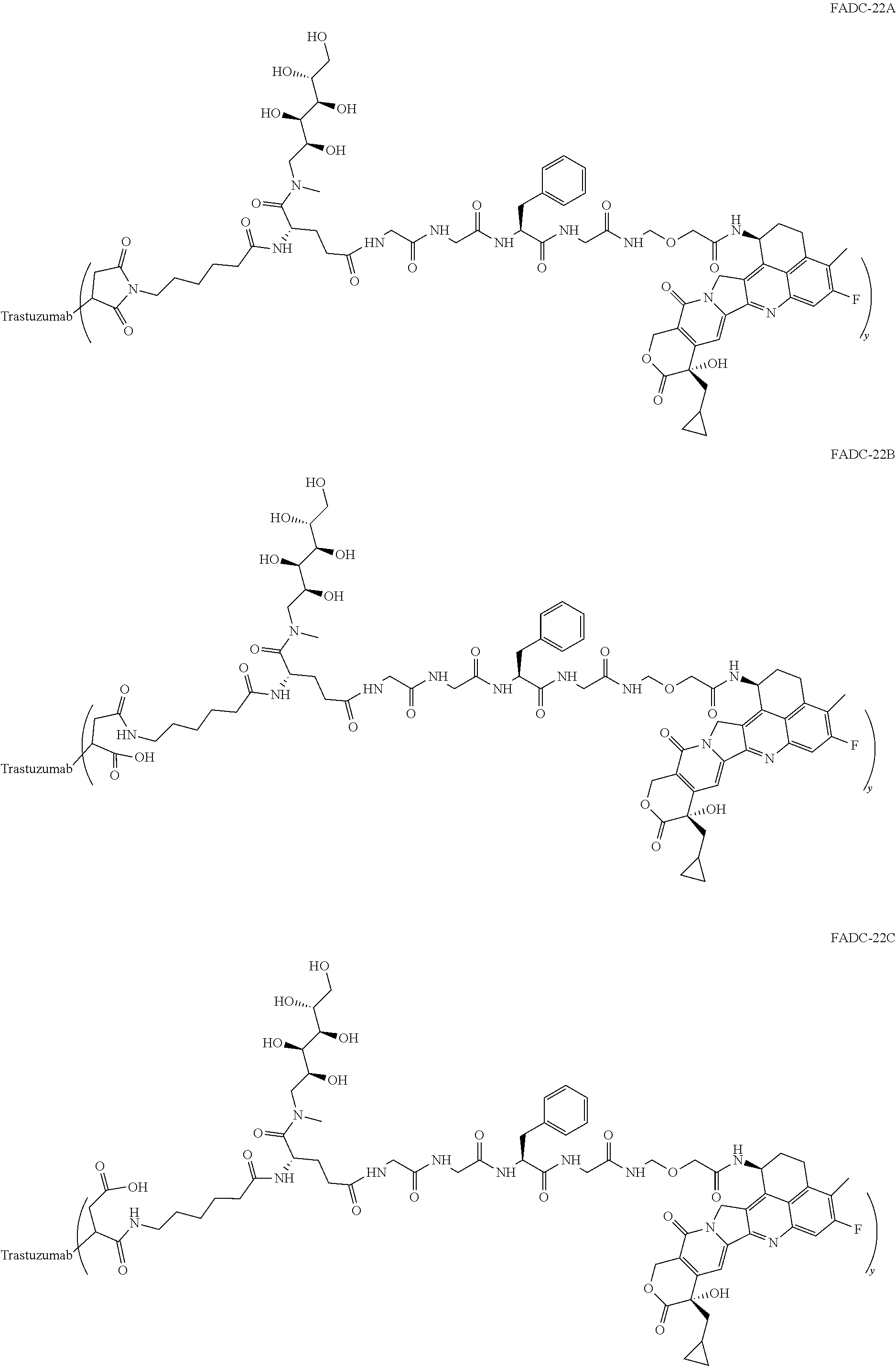

Using antibody Trastuzumab and LD-22 as starting materials, the preparation was carried out by applying the method disclosed in the patent application “WO2022068898A1”. The PBS buffer with the exemplary product ADC-22 of the conjugate mixture (mixture of FADC-22A and/or FADC-22B and/or FADC-22C) was obtained and stored at 4° C.

### Example 4-23: ADC-23 to Example 4-26: ADC-26

| | |
|---|---|
| 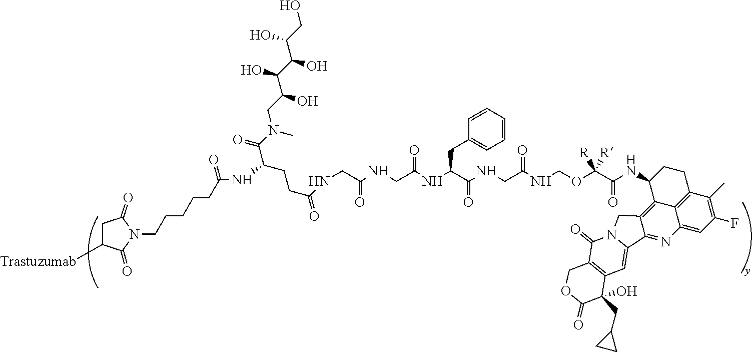 | FADC-X₅A |
| 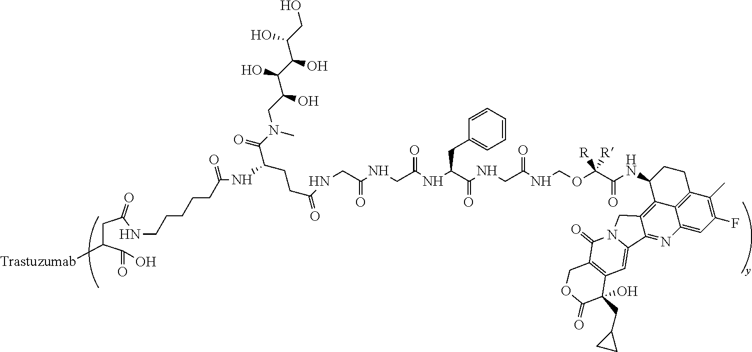 | FADC-X₅B |
| 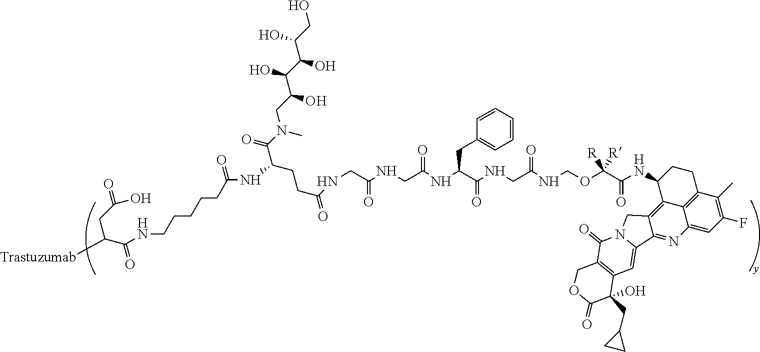 | FADC-X₅C |

| | Conjugate mixture of FADC-X₅A | | |
|---|---|---|---|
| Exemplary ADC | and/or FADC-X₅B and/or FADC- | | |
| product | X₅C | R | R′ |
| ADC-23 | FADC-23A and/or FADC-23B | —CH₃ | —H |
| | and/or FADC-23C | | |
| ADC-24 | FADC-24A and/or FADC-24B | —H | —CH₃ |
| | and/or FADC-24C | | |
| ADC-25 | FADC-25A and/or FADC-25B and/or FADC-25C | 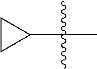 [SMILES: *C1CC1] | —H |
| ADC-26 | FADC-26A and/or FADC-26B and/or FADC-26C | —H | 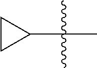 [SMILES: *C1CC1] |

LD-23, LD-24, LD-25 and LD-26 were used to replace LD-22, respectively, and the corresponding ADC-23, ADC-24, ADC-25 and ADC-26 were obtained by applying similar preparation methods, which were Examples 4-23 to 4-26.

### Example 4-27 ADC-27

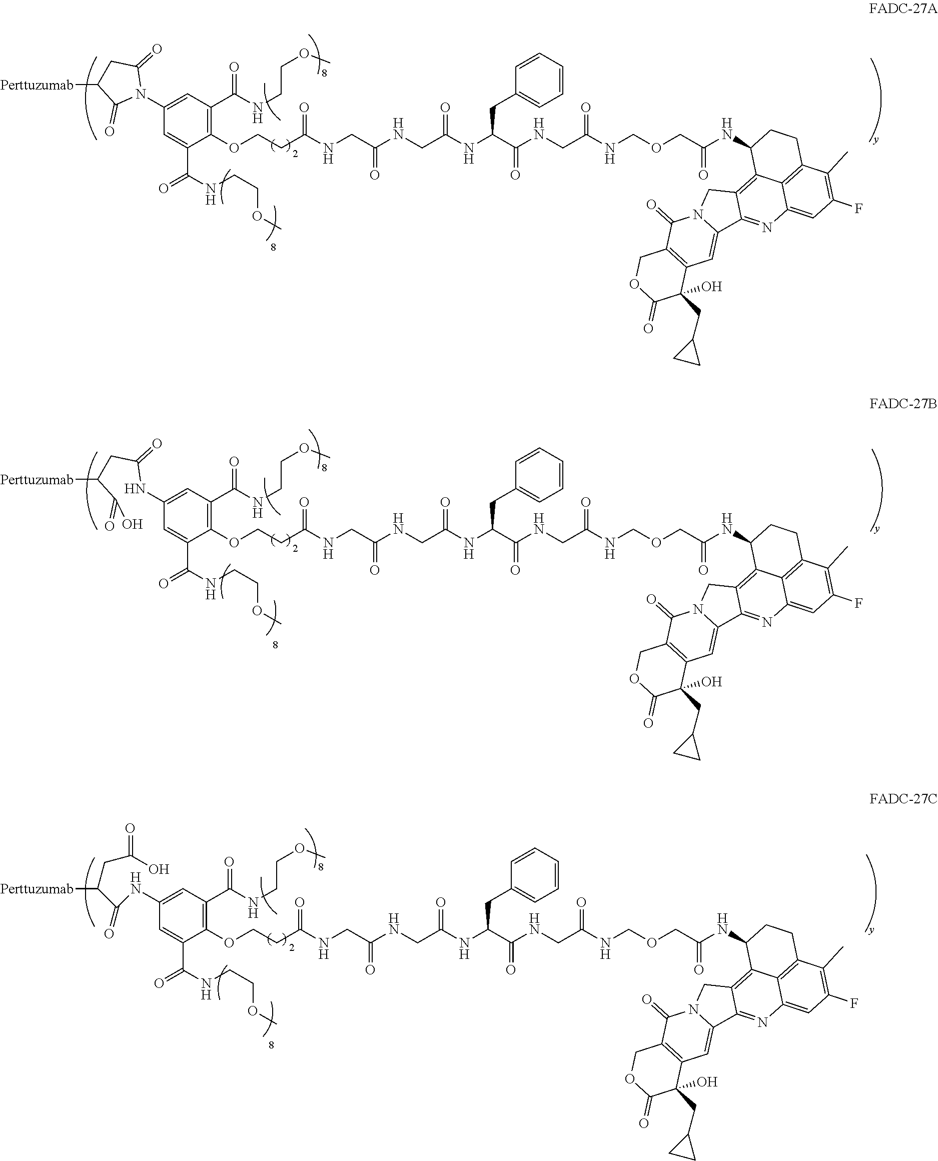

Using the antibody Pertuzumab and LD-1 as starting materials, the preparation was carried out by applying the method of step A. The PBS buffer with the exemplary product ADC-27 of the conjugate mixture (mixture of FADC-27A and/or FADC-27B and/or FADC-27C) was obtained and stored at 4° C.

### Example 4-28: ADC-28

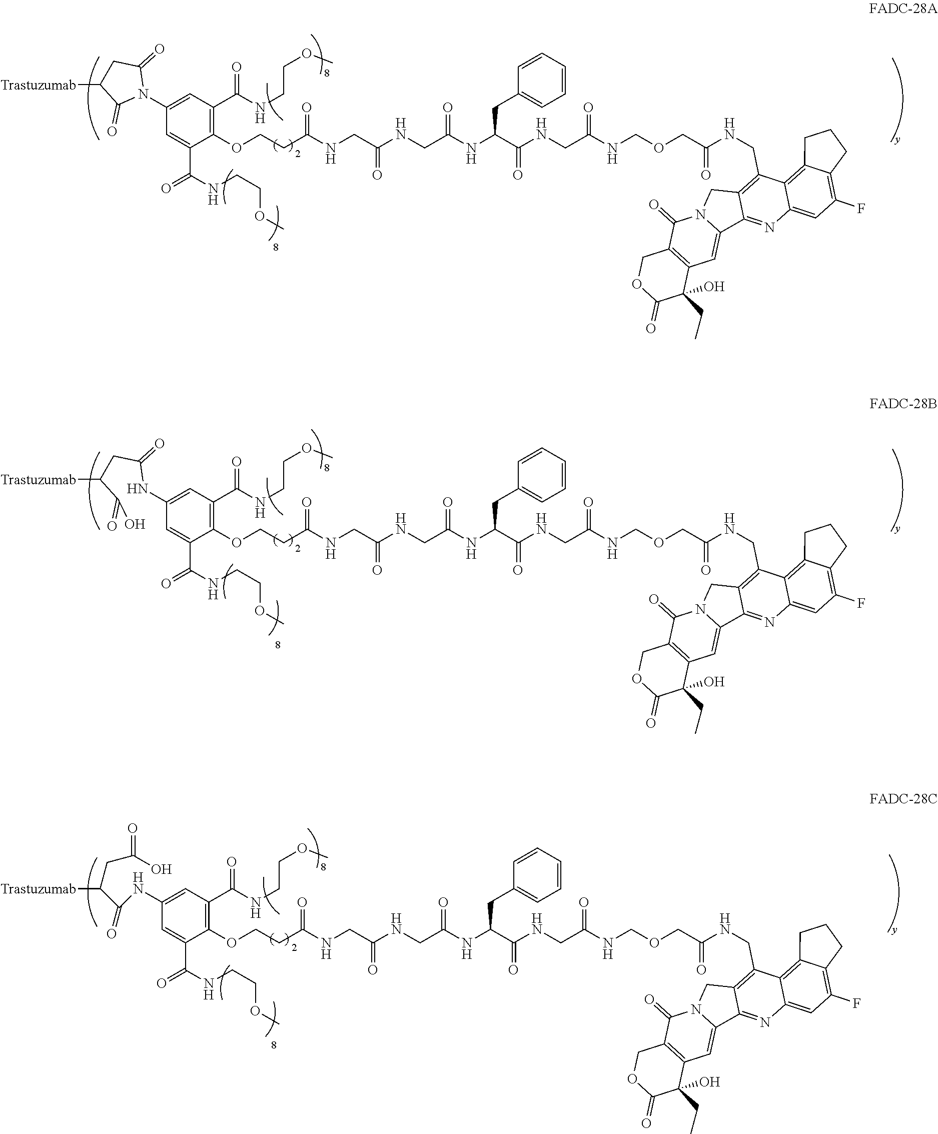

Using the antibody Trastuzumab and LD-27 as starting materials, the preparation was carried out by applying the method of step A. The PBS buffer with the exemplary product ADC-28 of the conjugate mixture (mixture of FADC-28A and/or FADC-28B and/or FADC-28C) was obtained and stored at 4° C.

### Example 4-29: ADC-29

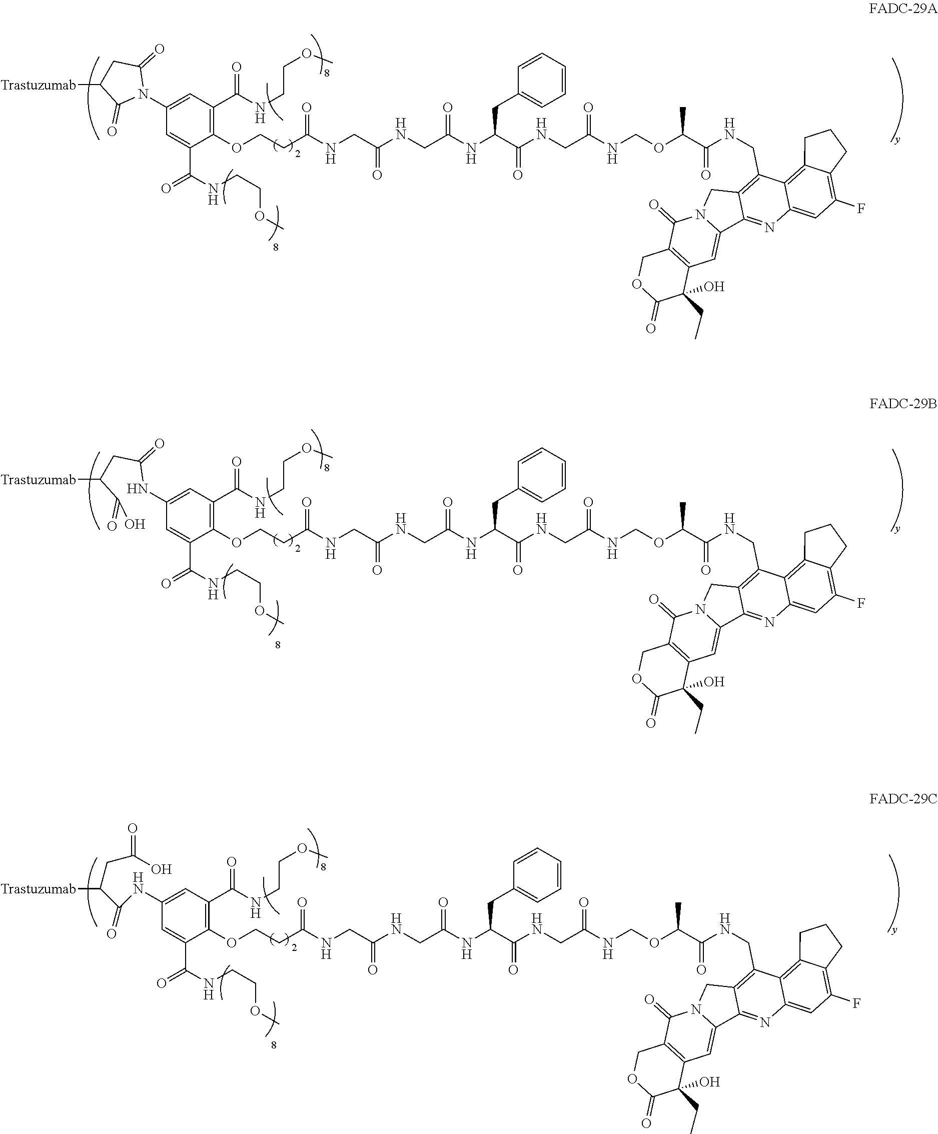

Using the antibody Trastuzumab and LD-28 as starting materials, the preparation was carried out by applying the method of step A. The PBS buffer with the exemplary product ADC-29 of the conjugate mixture (mixture of FADC-29A and/or FADC-29B and/or FADC-29C) was obtained and stored at 4° C.

### Example 4-30: ADC-30

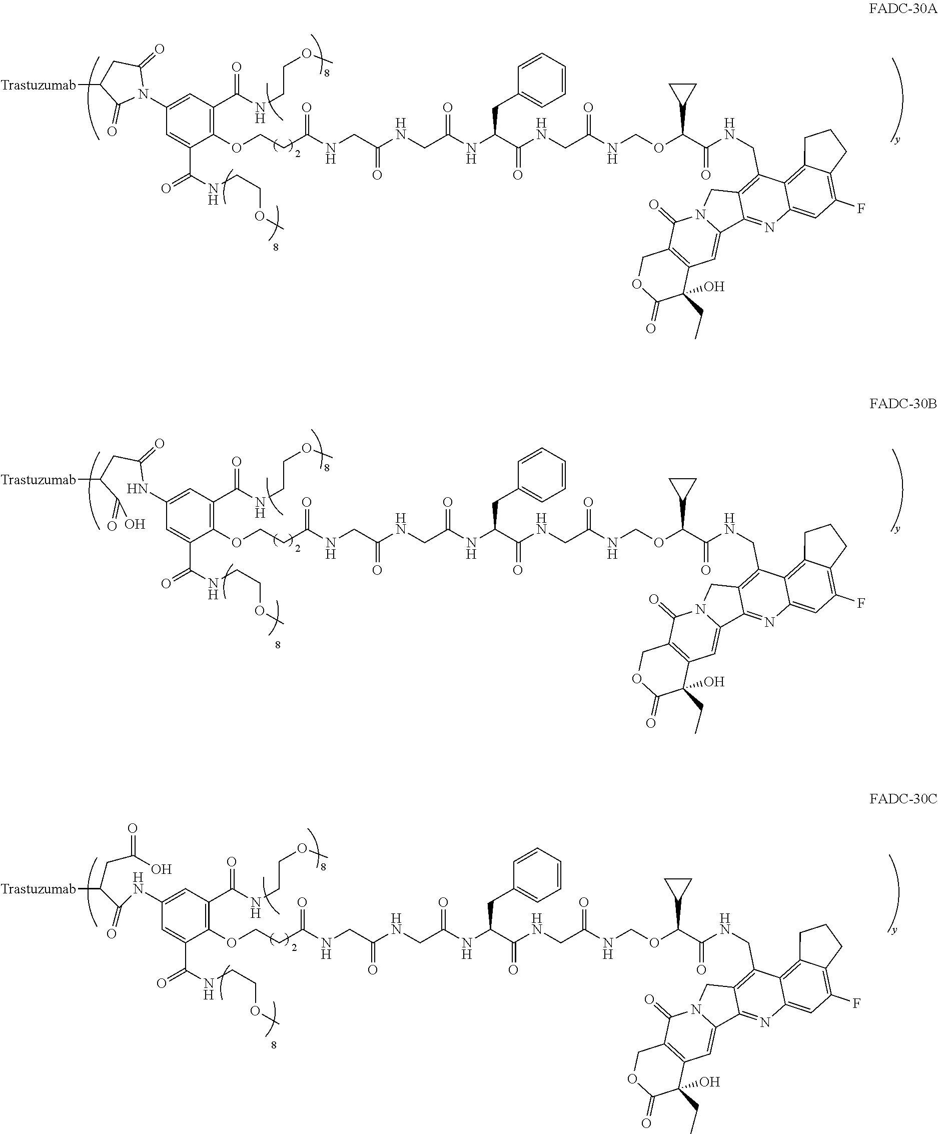

Using the antibody Trastuzumab and LD-29 as starting materials, the preparation was carried out by applying the method of step A. The PBS buffer with the exemplary product ADC-30 of the conjugate mixture (mixture of FADC-30A and/or FADC-30B and/or FADC-30C) was obtained and stored at 4° C.

### Example 4-31: ADC-31

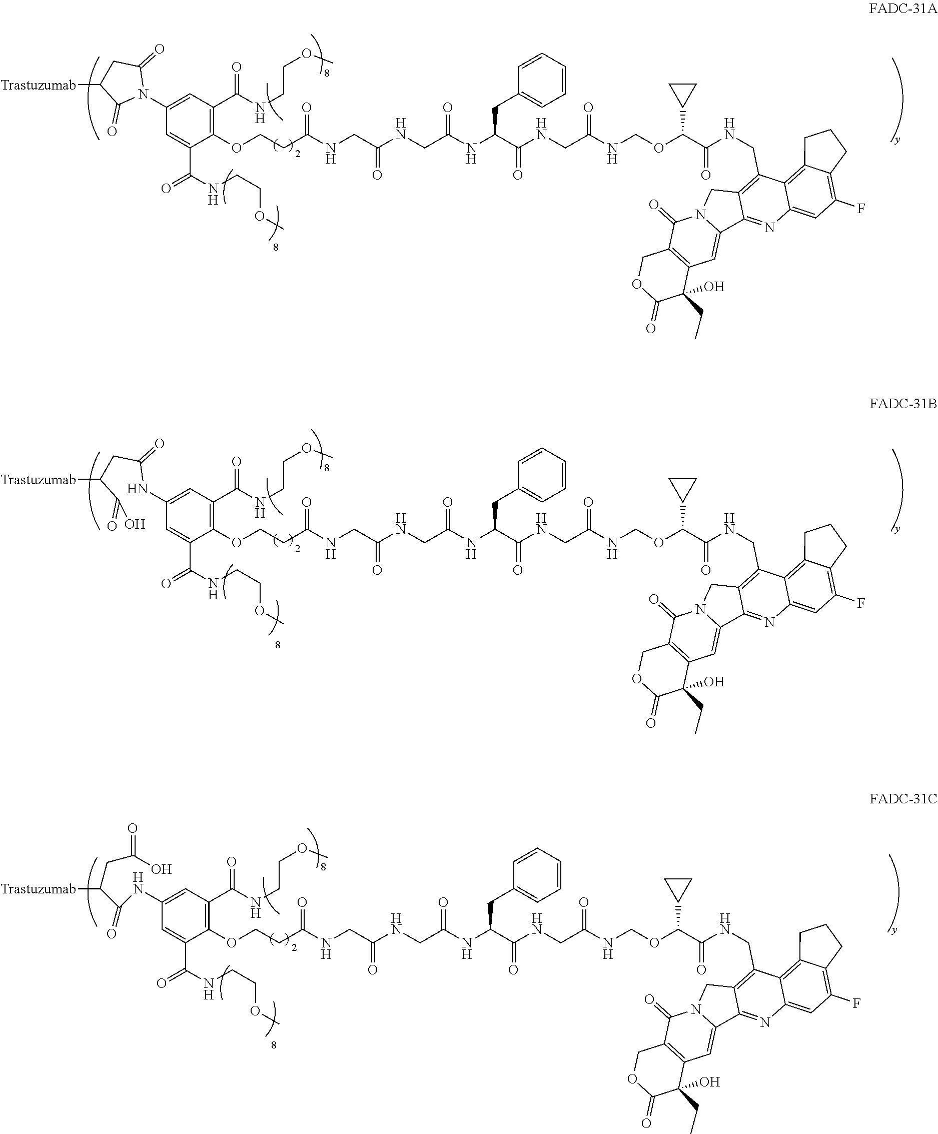

Using the antibody Trastuzumab and LD-30 as starting materials, the preparation was carried out by applying the method of step A. The PBS buffer with the exemplary product ADC-31 of conjugate mixture (mixture of FADC-31A and/or FADC-31B and/or FADC-31C) was obtained and stored at 4° C.

### Example 4-32: ADC-32 to Example 4-35: ADC-35

| | |
|---|---|
| 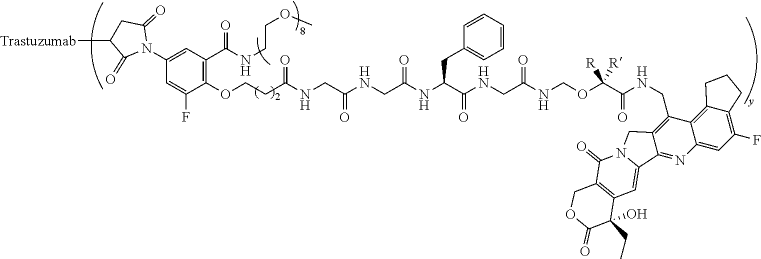 | FADC-X₆A |
| 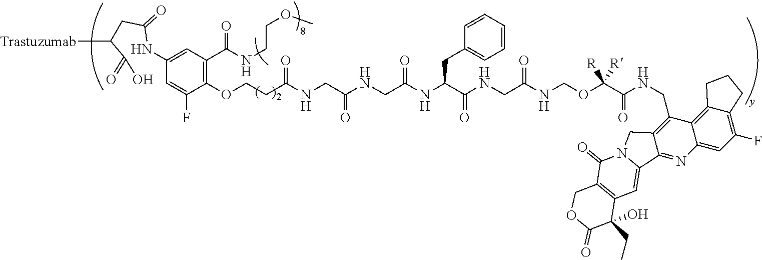 | FADC-X₆B |
| 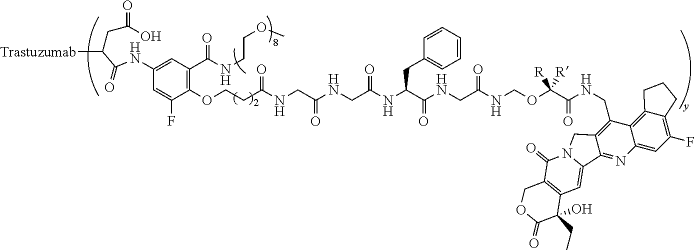 | FADC-X₆C |

| Exemplary ADC | Conjugate mixture of FADC-X₆A | | |
|---|---|---|---|
| product | and/or ADC-X₆B and/or FADC-X₆C | R | R′ |
| ADC-32 | FADC-32A and/or FADC-32B | —H | —H |
| | and/or FADC-32C | | |
| ADC-33 | FADC-33A and/or FADC-33B | —CH₃ | —H |
| | and/or FADC-33C | | |
| ADC-34 | FADC-34A and/or FADC-34B and/or FADC-34C | 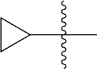 [SMILES: *C1CC1] | —H |
| ADC-35 | FADC-35A and/or FADC-35B and/or FADC-35C | —H | 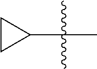 [SMILES: *C1CC1] |

The preparation was carried out by applying the method of step A using the antibody Trastuzumab with LD-31 to LD-34, respectively, and the PBS buffers with the exemplary products ADC-32 to ADC-35 of the corresponding conjugate mixture (mixture of FADC-A and/or FADC-B and/or FADC-C) were obtained and stored at 4° C., respectively, which were Examples 4-32 to 4-35.

### Example 4-36: ADC-36 to Example 4-38: ADC-38

| | |
|---|---|
| 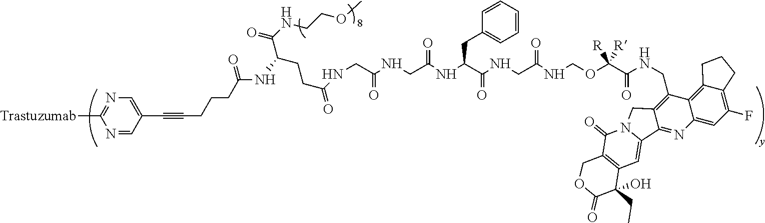 | FADC-X₇ |

| | General formula FADC-X₇ | |
|---|---|---|
| Exemplary ADC product | R | R′ |
| ADC-36 | —H | —H |
| ADC-37 | —CH₃ | —H |
| ADC-38 | 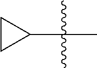 [SMILES: *C1CC1] | —H |

The preparation was carried out by applying the method disclosed in the patent application “WO2019114666A1” using the antibody Trastuzumab with LD-35 to LD-37, respectively, and the PBS buffers with the exemplary products ADC-36 to ADC-38 of the general formulas FADC-36 to FADC-38 were obtained and stored at 4° C., respectively, which were Examples 4-36 to 4-38.

### Example 4-39: ADC-39 to Example 4-42: ADC-42

| | |
|---|---|
| 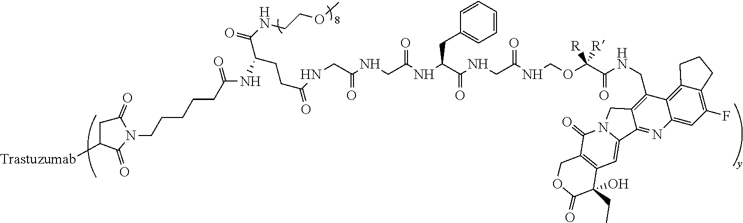 | FADC-X₈A |
| 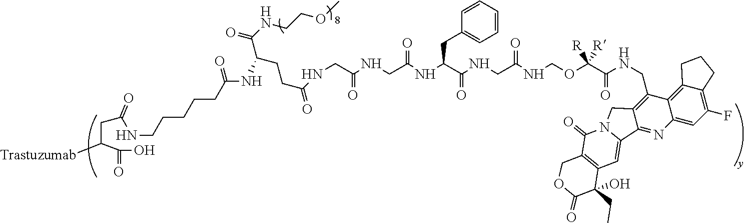 | FADC-X₈B |
| 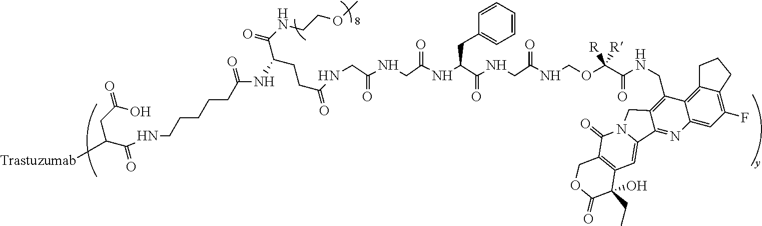 | FADC-X₈C |

| Exemplary ADC | Conjugate mixture of FADC-X₈A | | |
|---|---|---|---|
| product | and/or ADC-X₈B and/or FADC-X₈C | R | R′ |
| ADC-39 | FADC-39A and/or FADC-39B | —H | —H |
| | and/or FADC-39C | | |
| ADC-40 | FADC-40A and/or FADC-40B | —CH₃ | —H |
| | and/or FADC-40C | | |
| ADC-41 | FADC-41A and/or FADC-41B and/or FADC-41C | 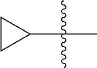 [SMILES: *C1CC1] | —H |
| ADC-42 | FADC-42A and/or FADC-42B and/or FADC-42C | —H | 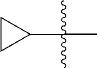 [SMILES: *C1CC1] |

The preparation was carried out by applying the method disclosed in the patent application “WO2022068898A1” using the antibody Trastuzumab with LD-38 to LD-41, respectively, and the PBS buffers with the exemplary products ADC-39 to ADC-42 of the corresponding conjugate mixture (mixture of FADC-A and/or FADC-B and/or FADC-C) were obtained and stored at 4° C., respectively, which were Examples 4-39 to 4-42.

### Example 4-43: ADC-43 to Example 4-45: ADC-45

| | |
|---|---|
| 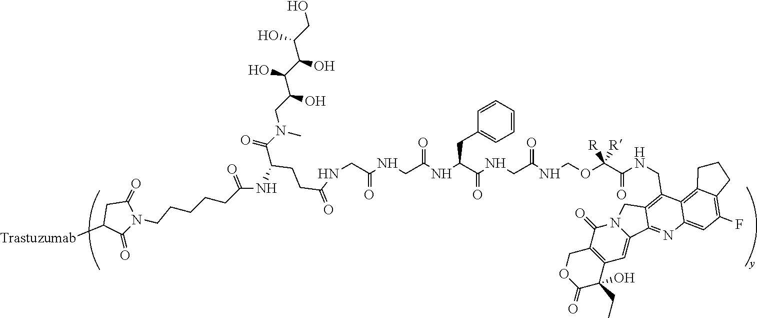 | FADC-X₉A |
| 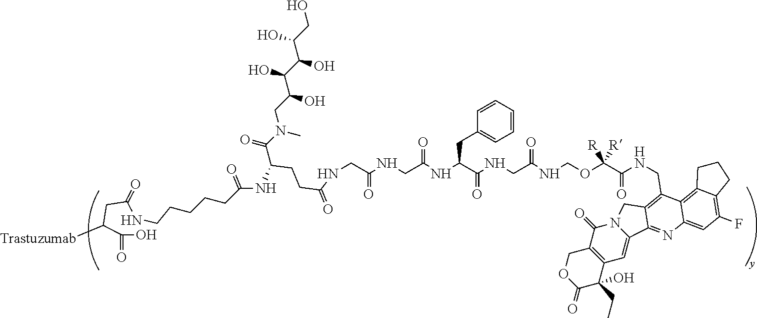 | FADC-X₉B |
| 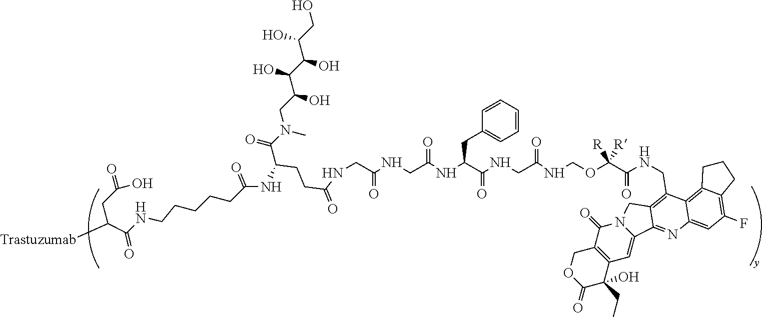 | FADC-X₉C |

| Exemplary ADC | Conjugate mixture of FADC-X₉A | | |
|---|---|---|---|
| product | and/or FADC-X₉B and/or FADC-X₉C | R | R′ |
| ADC-43 | FADC-43A and/or FADC-43B and/or | —H | —H |
| | FADC-43C | | |
| ADC-44 | FADC-44A and/or FADC-44B and/or | —CH₃ | —H |
| | FADC-44C | | |
| ADC-45 | FADC-45A and/or FADC-45B and/or FADC-45C | 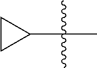 [SMILES: *C1CC1] | —H |

The preparation was carried out by applying the method disclosed in the patent application “WO2022068898A1” using the antibody Trastuzumab with LD-42 to LD-44, respectively, and the PBS buffers with the exemplary products ADC-43 to ADC-45 of the corresponding conjugate mixture (mixture of FADC-A and/or FADC-B and/or FADC-C) were obtained and stored at 4° C., respectively, which were Examples 4-43 to 4-45.

### Example 4-46: ADC-46 to Example 4-48: ADC-48

| | |
|---|---|
| 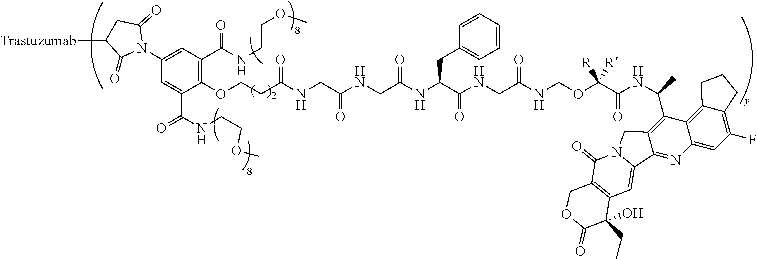 | FADC-X₁₀A |
| 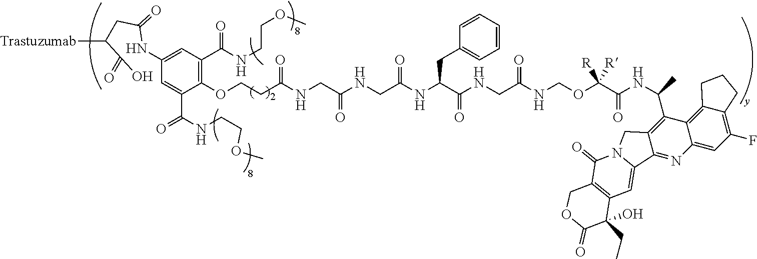 | FADC-X₁₀B |
| 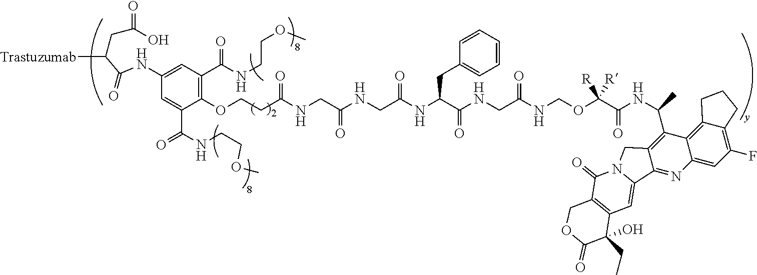 | FADC-X₁₀C |

| Exemplary ADC | Conjugate mixture of FADC-x10A | | |
|---|---|---|---|
| product | and/or FADC-x10B and/or FADC-x10C | R | R′ |
| ADC-46 | FADC-46A and/or FADC-46B and/or | —H | —H |
| | FADC-46C | | |
| ADC-47 | FADC-47A and/or FADC-47B and/or FADC-47C | 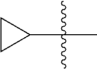 [SMILES: *C1CC1] | —H |
| ADC-48 | FADC-48A and/or FADC-48B and/or FADC-48C | —H | 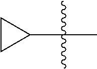 [SMILES: *C1CC1] |

The preparation was carried out by applying the method of step A using the antibody Trastuzumab with LD-45 to LD-47, respectively, and the PBS buffers with the exemplary products ADC-46 to ADC-48 of the corresponding conjugate mixture (mixture of FADC-A and/or FADC-B and/or FADC-C) were obtained and stored at 4° C., respectively, which were Examples 4-46 to 4-48.

### Example 4-49: ADC-49 to Example 4-51: ADC-51

| | |
|---|---|
| 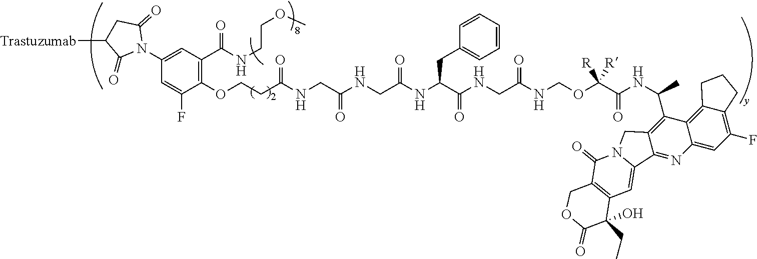 | FADC-X₁₁A |
| 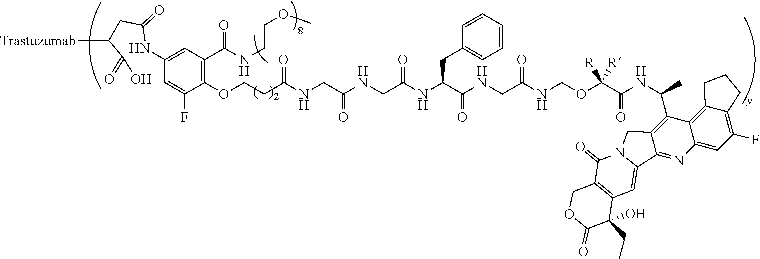 | FADC-X₁₁B |
| 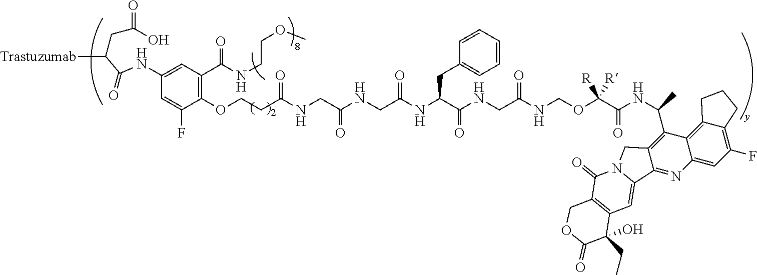 | FADC-X₁₁C |

| Exemplary ADC | Conjugate mixture of FADC-X₁₁A | | |
|---|---|---|---|
| product | and/or FADC-X₁₁B and/or FADC-X₁₁C | R | R′ |
| ADC-49 | FADC-49A and/or FADC-49B and/or | —H | —H |
| | FADC-49C | | |
| ADC-50 | FADC-50A and/or FADC-50B and/or FADC-50C | 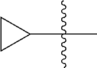 [SMILES: *C1CC1] | —H |
| ADC-51 | FADC-51A and/or FADC-51B and/or FADC-51C | —H | 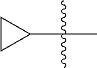 [SMILES: *C1CC1] |

The preparation was carried out by applying the method of step A using the antibody Trastuzumab with LD-48 to LD-50, respectively, and the PBS buffers with the exemplary products ADC-49 to ADC-51 of the corresponding conjugate mixture (mixture of FADC-A and/or FADC-B and/or FADC-C) were obtained and stored at 4° C., respectively, which were Examples 4-49 to 4-51.

### Example 4-52: ADC-52 to Example 4-54: ADC-54

| | |
|---|---|
| 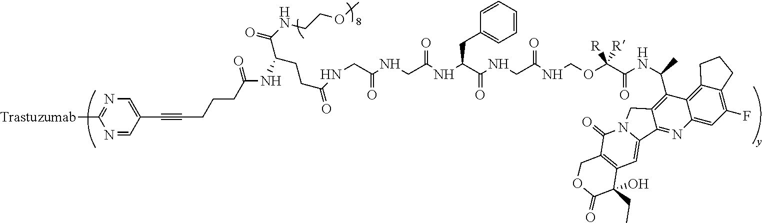 | FADC-X₁₂ |

| | General formula FADC-X₁₂ | |
|---|---|---|
| Exemplary ADC product | R | R′ |
| ADC-52 | —H | —H |
| ADC-53 | 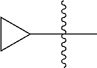 [SMILES: *C1CC1] | —H |
| ADC-54 | —H | 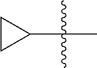 [SMILES: *C1CC1] |

The preparation was carried out by applying the method disclosed in the patent application “WO2019114666A1” using the antibody Trastuzumab with LD-51 to LD-53, respectively, and the PBS buffers with the exemplary products ADC-52 to ADC-54 of the general formulas FADC-52 to FADC-54 were obtained and stored at 4° C., respectively, which were Examples 4-52 to 4-54.

### Example 4-55: ADC-55 to Example 4-57: ADC-57

| | |
|---|---|
| 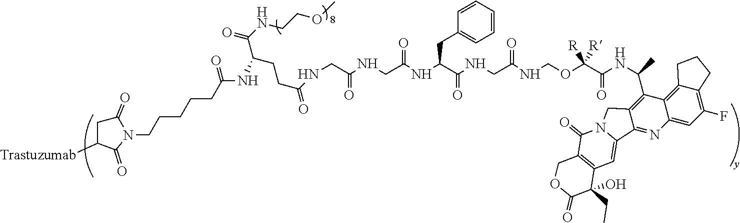 | FADC-X₁₃A |
| 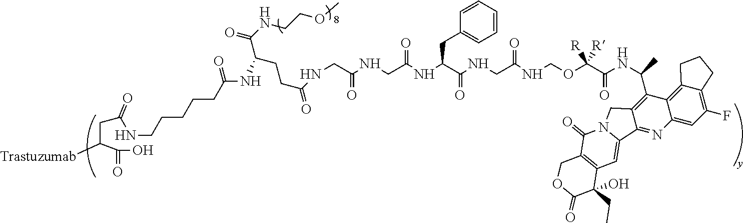 | FADC-X₁₃B |
| 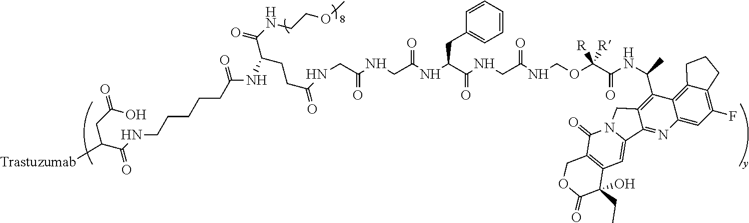 | FADC-X₁₃C |

| Exemplary ADC | Conjugate mixture of FADC-X₁₃A | | |
|---|---|---|---|
| product | and/or FADC-X₁₃B and/or FADC-X₁₃C | R | R′ |
| ADC-55 | FADC-55A and/or FADC-55B and/or | —H | —H |
| | FADC-55C | | |
| ADC-56 | FADC-56A and/or FADC-56B and/or FADC-56C | 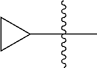 [SMILES: *C1CC1] | —H |
| ADC-57 | FADC-57A and/or FADC-57B and/or FADC-57C | —H | 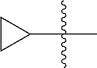 [SMILES: *C1CC1] |

The preparation was carried out by applying the method disclosed in patent application “WO2022068898A1” using the antibody Trastuzumab with LD-54 to LD-56, respectively, and the PBS buffers with the exemplary products ADC-55 to ADC-57 of the corresponding conjugate mixture (mixture of FADC-A and/or FADC-B and/or FADC-C) were obtained and stored at 4° C., respectively, which were Examples 4-55 to 4-57.

### Example 4-58: ADC-58 to Example 4-60: ADC-60

| | |
|---|---|
| 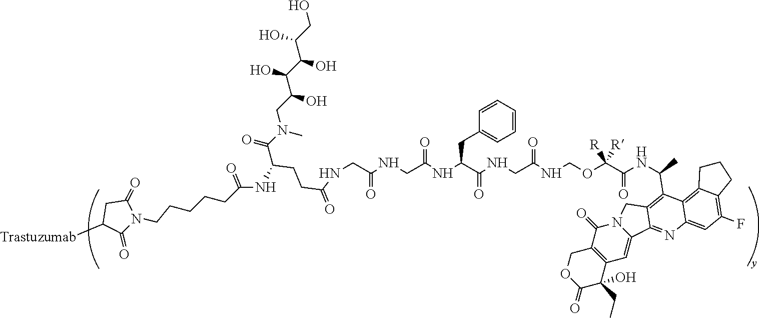 | FADC-X₁₄A |
| 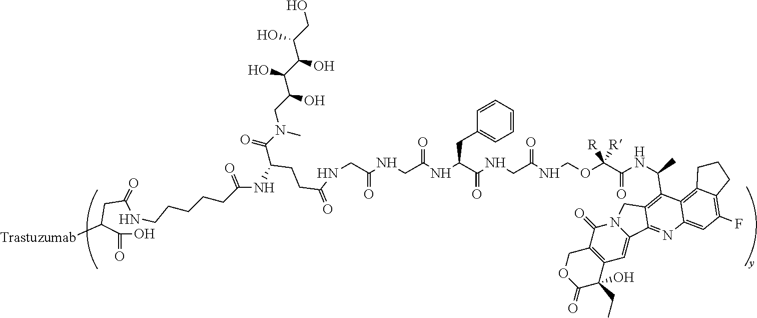 | FADC-X₁₄B |
| 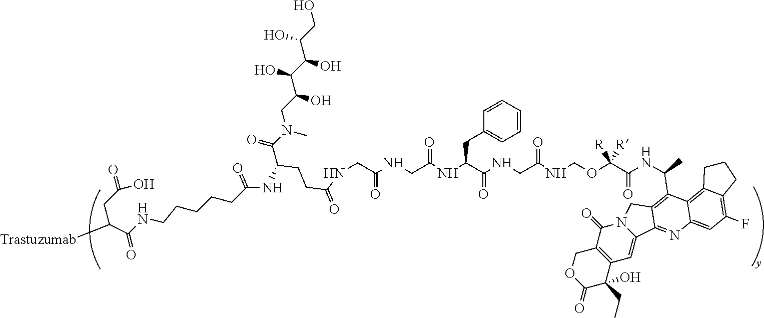 | FADC-X₁₄C |

| Exemplary ADC | Conjugate mixture of FADC-X₁₄A and/or | | |
|---|---|---|---|
| product | FADC-X₁₄B and/or FADC-X₁₄C | R | R′ |
| ADC-58 | FADC-58A and/or FADC-58B and/or | —H | —H |
| | FADC-58C | | |
| ADC-59 | FADC-59A and/or FADC-59B and/or FADC-59C | 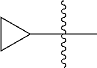 [SMILES: *C1CC1] | —H |
| ADC-60 | FADC-60A and/or FADC-60B and/or FADC-60C | —H | 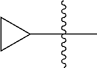 [SMILES: *C1CC1] |

The preparation was carried out by applying the method disclosed in patent application “WO2022068898A1” using the antibody Trastuzumab with LD-57 to LD-59, respectively, and the PBS buffers with the exemplary products ADC-58 to ADC-60 of the corresponding conjugate mixture (mixture of FADC-A and/or FADC-B and/or FADC-C) were obtained and stored at 4° C., respectively, which were Examples 4-58 to 4-60.

### Example 4-61: ADC-61

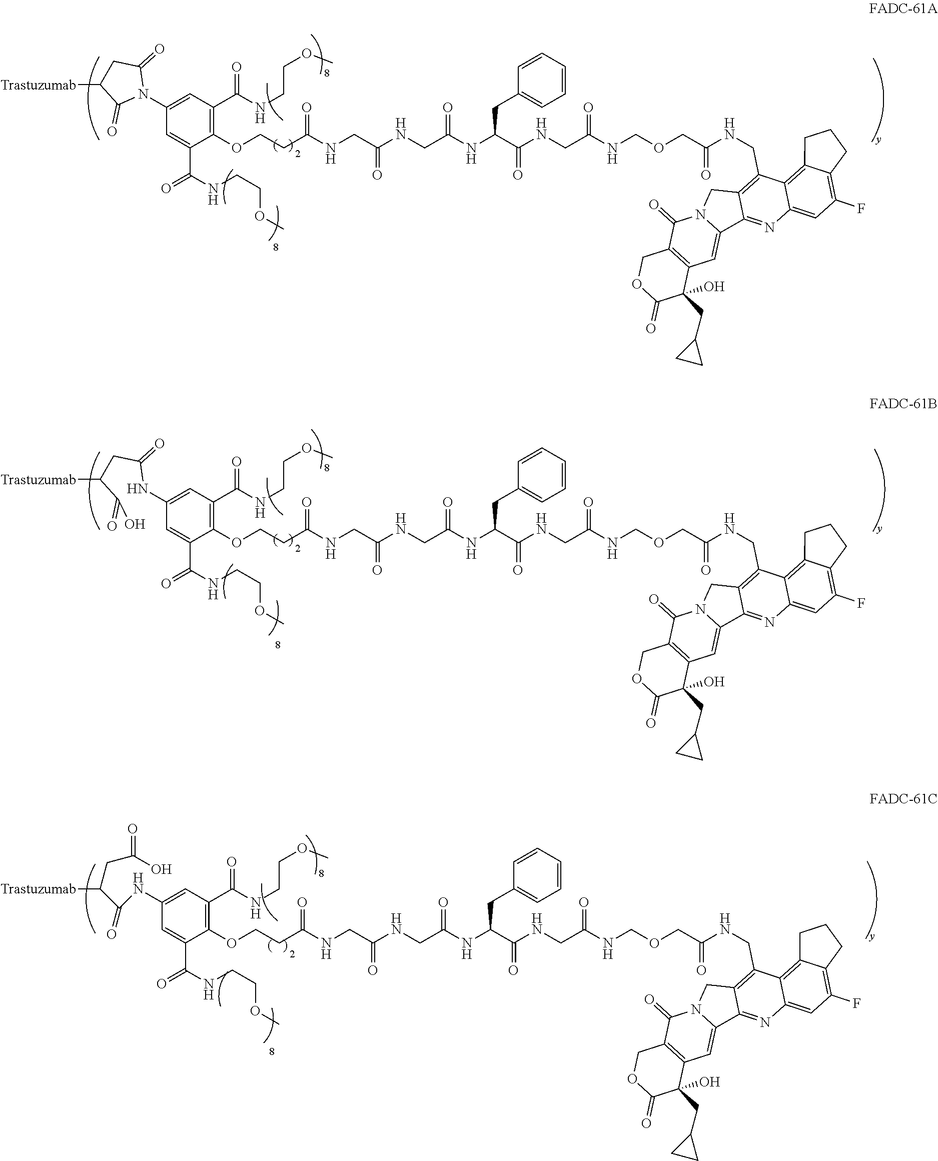

Using the antibody Trastuzumab and LD-60 as starting materials, the preparation was carried out by applying the method of step A. The PBS buffer with the exemplary product ADC-61 of the conjugate mixture (mixture of FADC-61A and/or FADC-61B and/or FADC-61C) was obtained and stored at 4° C.

### Example 4-62: ADC-62

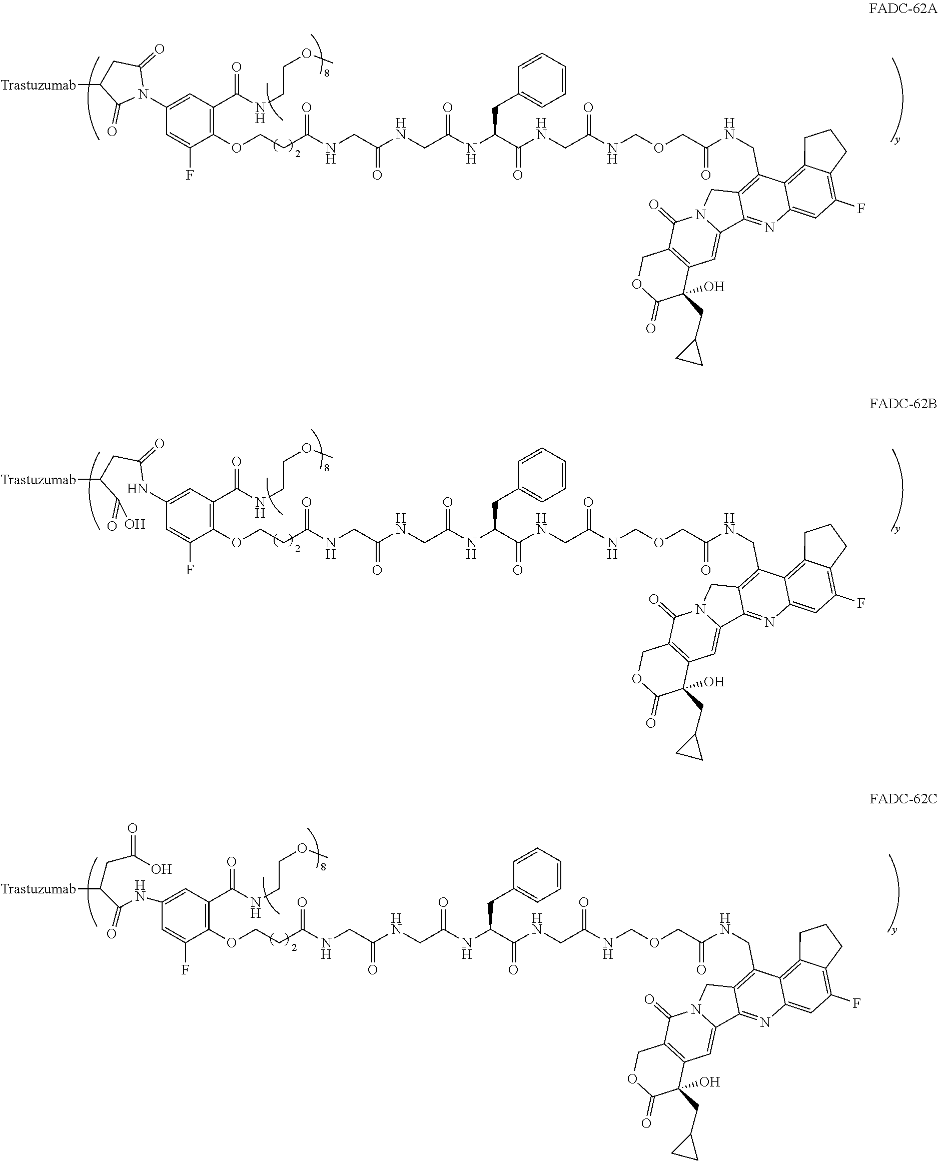

Using the antibody Trastuzumab and LD-61 as starting materials, the preparation was carried out by applying the method of step A. The PBS buffer with the exemplary product ADC-62 of the conjugate mixture (mixture of FADC-62A and/or FADC-62B and/or FADC-62C) was obtained and stored at 4° C.

### Example 4-63: ADC-63

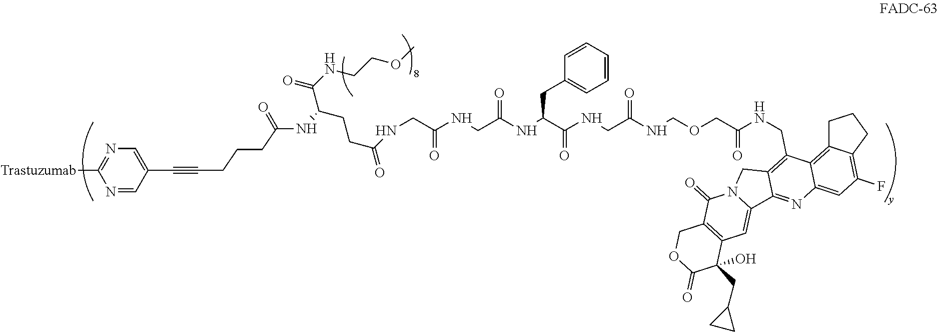

Using the antibody Trastuzumab and LD-62 as starting materials, the preparation was carried out by applying the method disclosed in the patent application “WO2019114666A1”. The PBS buffer with the exemplary product ADC-63 of the general formula of FADC-63 was obtained and stored at 4° C.

### Example 4-64: ADC-64

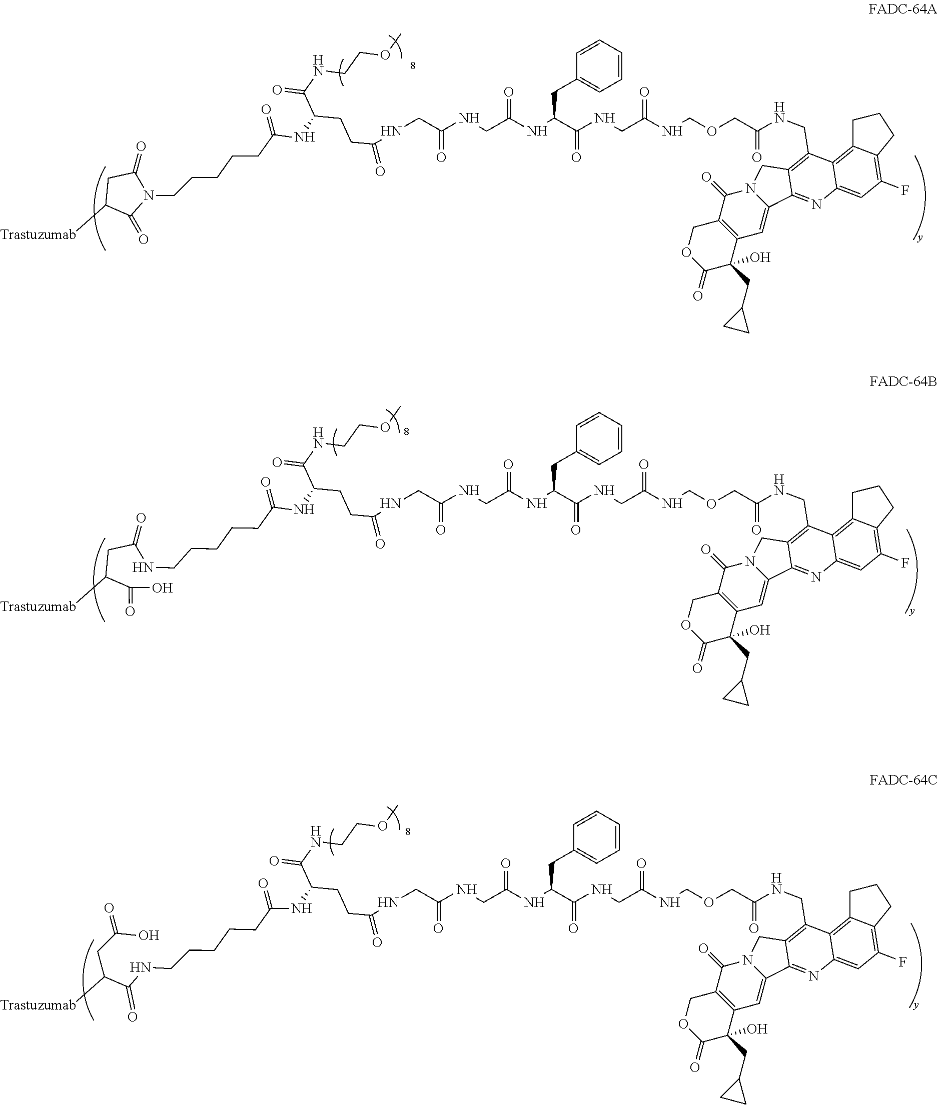

Using the antibody Trastuzumab and LD-63 as starting materials, the preparation was carried out by applying the method disclosed in the patent application “WO2022068898A1”. The PBS buffer with the exemplary product ADC-64 of the conjugate mixture (mixture of FADC-64A and/or FADC-64B and/or FADC-64C) was obtained and stored at 4° C.

### Example 4-65: ADC-65

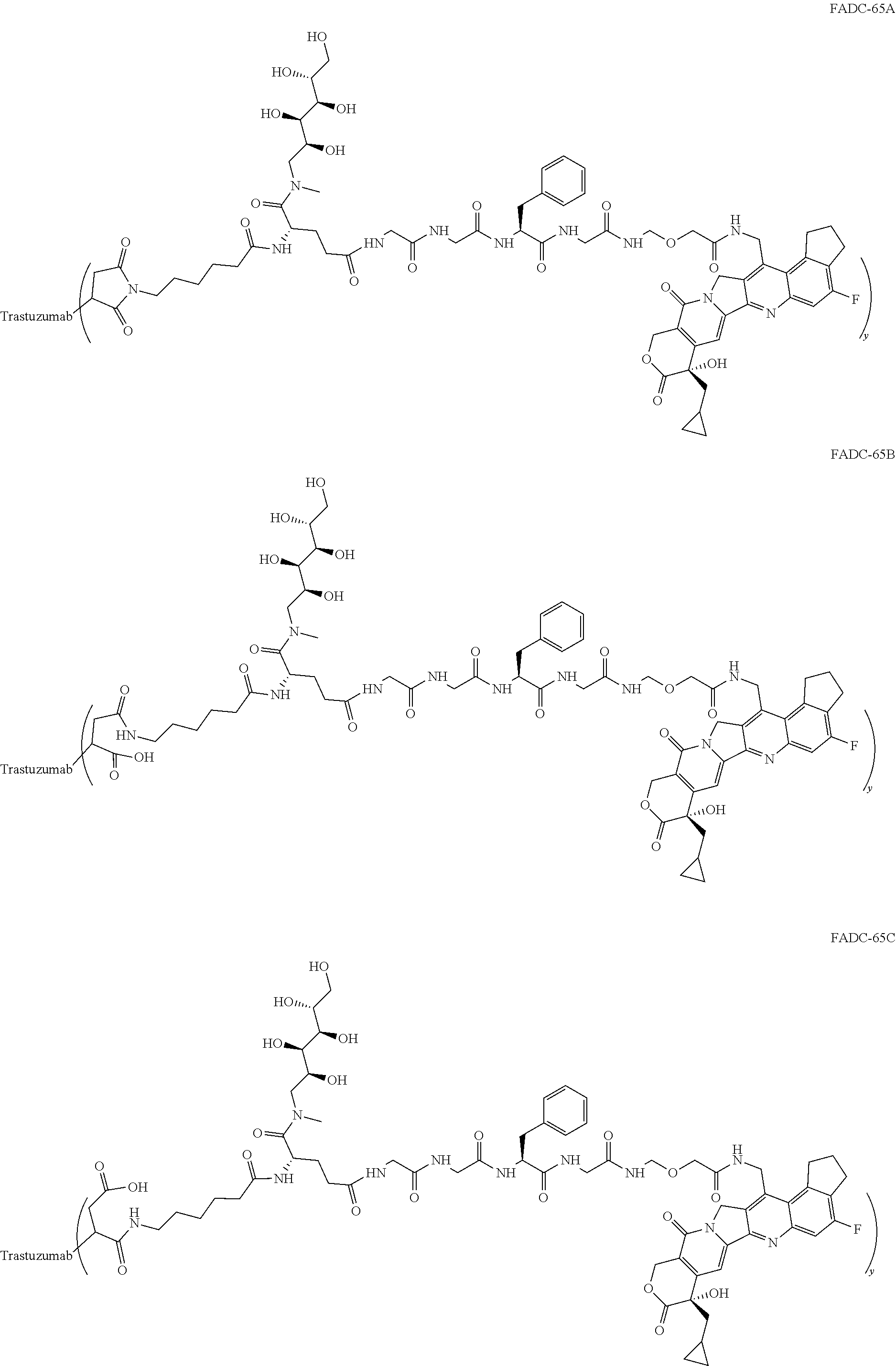

Using the antibody Trastuzumab and LD-64 as starting materials, the preparation was carried out by applying the method disclosed in the patent application “WO2022068898A1”. The PBS buffer with the exemplary product ADC-65 of the conjugate mixture (mixture of FADC-65A and/or FADC-65B and/or FADC-65C) was obtained and stored at 4° C.

### Example 4-66: ADC-66

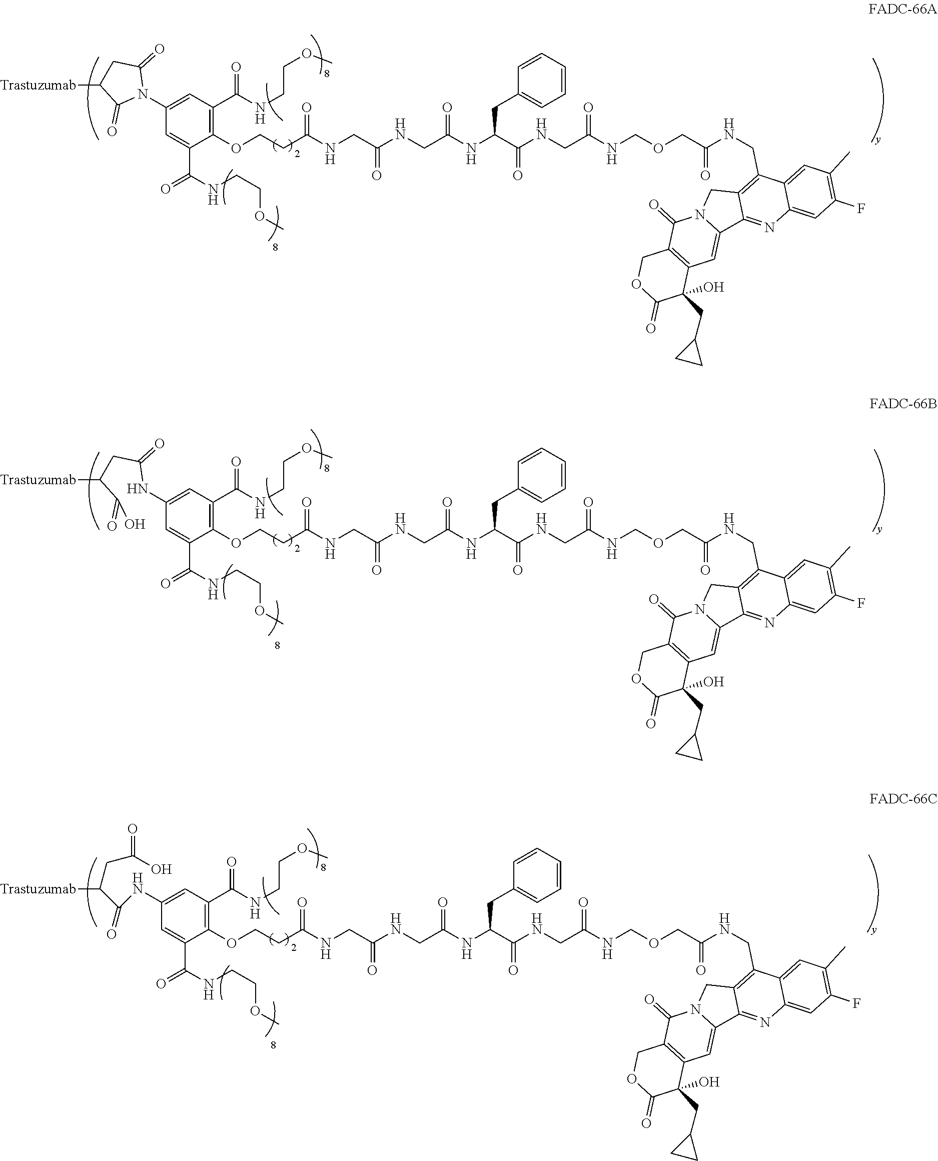

Using the antibody Trastuzumab and LD-65 as starting materials, the preparation was carried out by applying the method of step A. The PBS buffer with the exemplary product ADC-66 of the conjugate mixture (mixture of FADC-66A and/or FADC-66B and/or FADC-66C) was obtained and stored at 4° C.

### Example 4-67: ADC-67 to Example 4-75: ADC-75

| | |
|---|---|
| 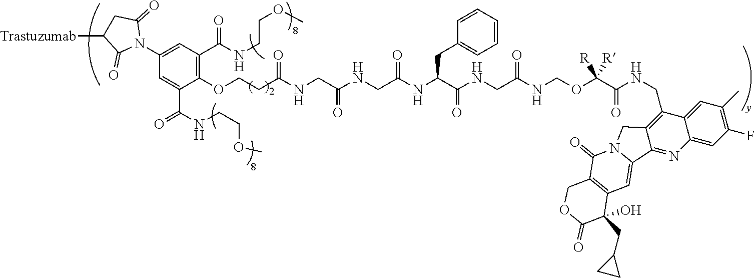 | FADC-X₁₅A |
| 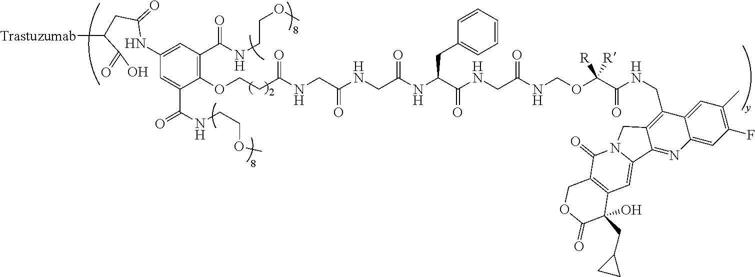 | FADC-X₁₅B |
| 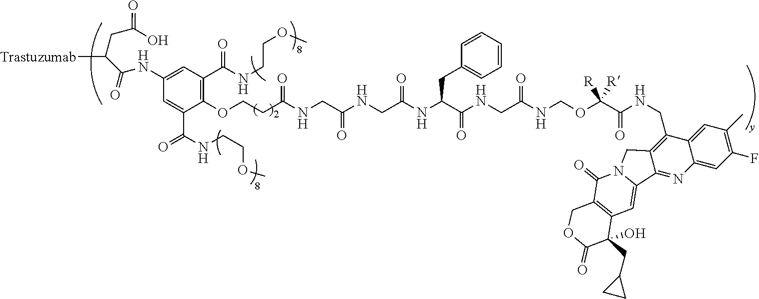 | FADC-X₁₅C |

| Exemplary ADC | Conjugate mixture of FADC-X₁₅A | | |
|---|---|---|---|
| product | and/or FADC-X₁₅B and/or FADC-X₁₅C | R | R′ |
| ADC-67 | FADC-67A and/or FADC-67B and/or | —CH₃ | —H |
| | FADC-67C | | |
| ADC-68 | FADC-68A and/or FADC-68B and/or | —H | —CH₃ |
| | FADC-68C | | |
| ADC-69 | FADC-69A and/or FADC-69B and/or FADC-69C | 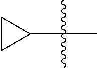 [SMILES: *C1CC1] | —H |
| ADC-70 | FADC-70A and/or FADC-70B and/or FADC-70C | —H | 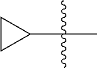 [SMILES: *C1CC1] |

| | |
|---|---|
| 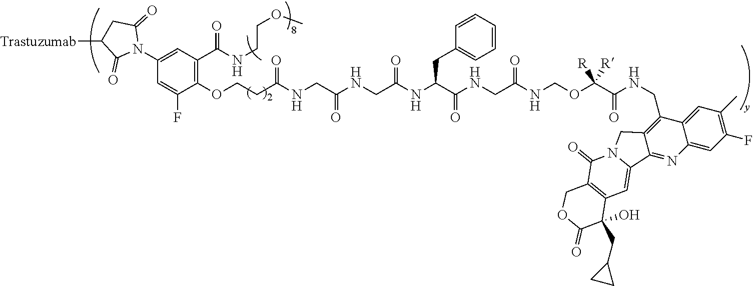 | FADC-X₁₆A |
| 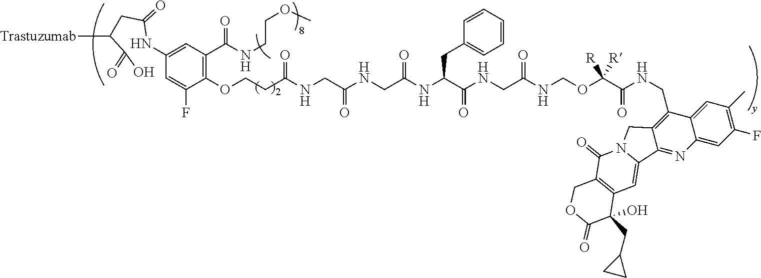 | FADC-X₁₆B |
| 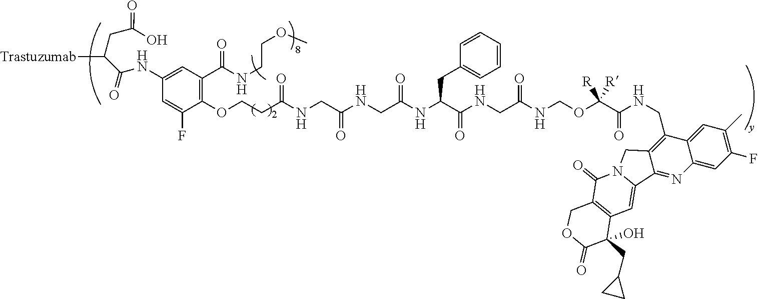 | FADC-X₁₆C |

| Exemplary ADC | Conjugate mixture of FADC-X₁₆A | | |
|---|---|---|---|
| product | and/or FADC-X₁₆B and/or FADC-X₁₆C | R | R′ |
| ADC-71 | FADC-71A and/or FADC-71B and/or | —H | —H |
| | FADC-71C | | |
| ADC-72 | FADC-72A and/or FADC-72B and/or | —CH₃ | —H |
| | FADC-72C | | |
| ADC-73 | FADC-73A and/or FADC-73B and/or | —H | —CH₃ |
| | FADC-73C | | |
| ADC-74 | FADC-74A and/or FADC-74B and/or FADC-74C | 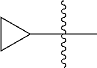 [SMILES: *C1CC1] | —H |
| ADC-75 | FADC-75A and/or FADC-75B and/or FADC-75C | —H | 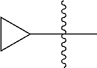 [SMILES: *C1CC1] |

LD-66 to LD-74 were used instead of LD-65, respectively, and similar preparation methods were used to obtain the corresponding ADC-67 to ADC-75, which were Examples 4-67 to 4-75.

### Example 4-76: ADC-76 to Example 4-80: ADC-80

| | |
|---|---|
| 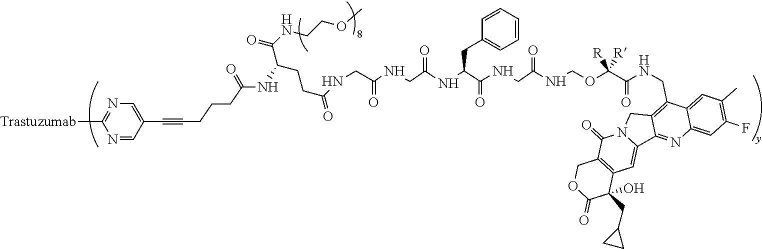 | FADC-X₁₇ |

| | General formula FADC-X₁₇ | |
|---|---|---|
| Exemplary ADC product | R | R′ |
| ADC-76 | —H | —H |
| ADC-77 | —CH₃ | —H |
| ADC-78 | —H | —CH₃ |
| ADC-79 | 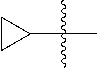 [SMILES: *C1CC1] | —H |
| ADC-80 | —H | 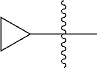 [SMILES: *C1CC1] |

Using the antibody Trastuzumab and LD-75 to LD-79 as starting materials, respectively, the preparation was carried out by applying the method disclosed in the patent application “WO2019114666A1”. PBS buffers with exemplary products ADC-76 to ADC-80 of the general formulas FADC-76 to FADC-80 were obtained, respectively, and stored at 4° C.

### Example 4-81: ADC-81

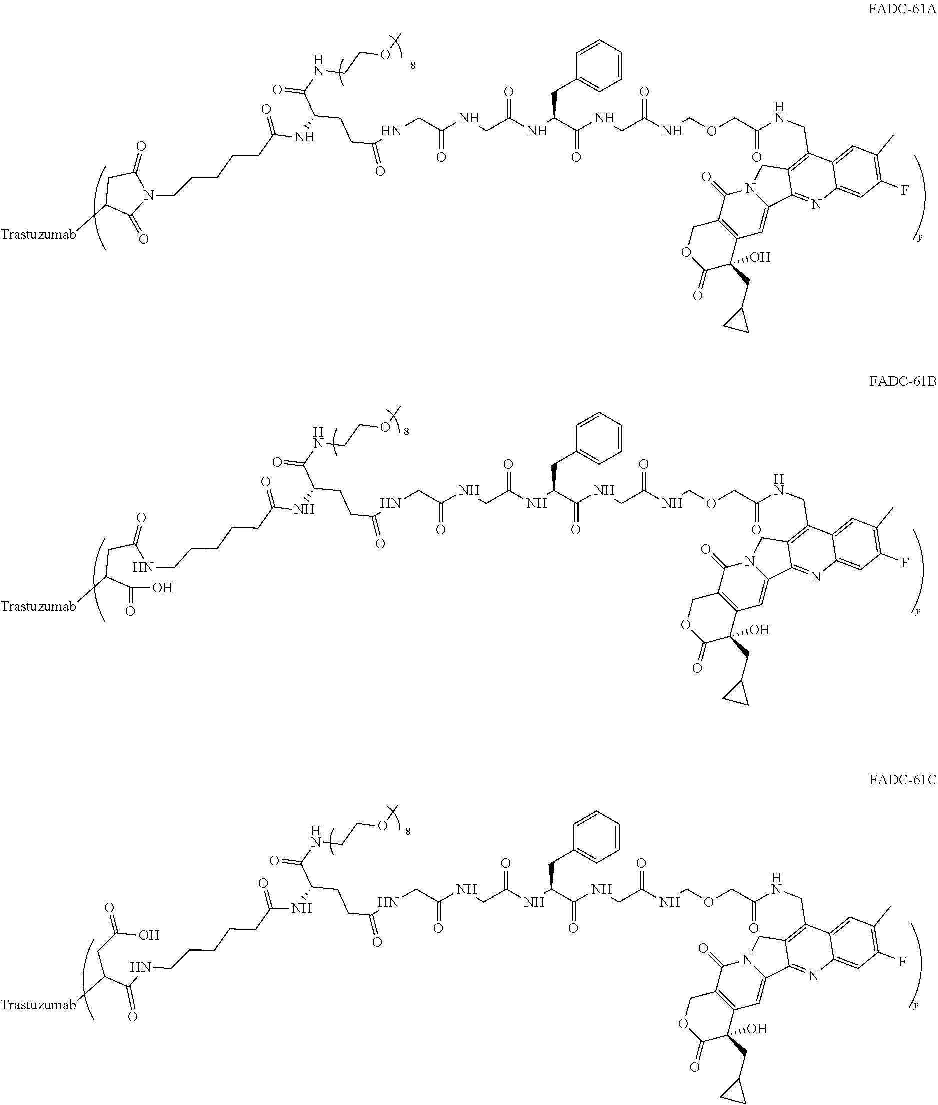

Using the antibody Trastuzumab and LD-80 as starting materials, the preparation was carried out by applying the method disclosed in the patent application “WO2022068898A1”. The PBS buffer with the exemplary product ADC-81 of the conjugate mixture (mixture of FADC-81A and/or FADC-81B and/or FADC-81C) was obtained and stored at 4° C.

### Example 4-82: ADC-82 to Example 4-85: ADC-85

| | |
|---|---|
| 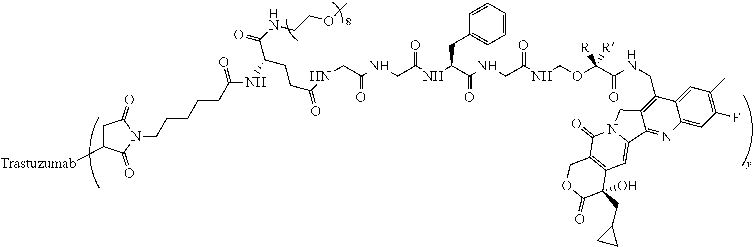 | FADC-X₁₈A |
| 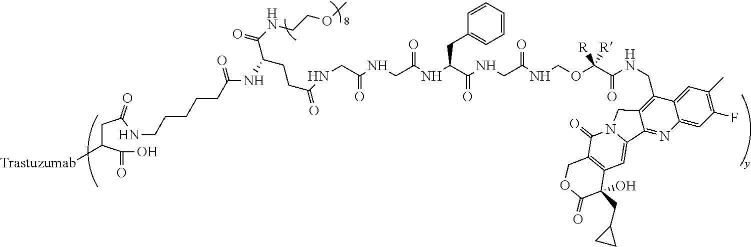 | FADC-X₁₈B |
| 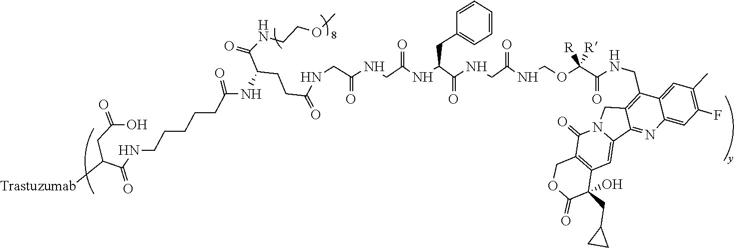 | FADC-X₁₈C |

| Exemplary ADC | Conjugate mixture of FADC-X₁₈A | | |
|---|---|---|---|
| product | and/or FADC-X₁₈B and/or FADC-X₁₈C | R | R′ |
| ADC-82 | FADC-82A and/or FADC-82B and/or | —CH₃ | —H |
| | FADC-82C | | |
| ADC-83 | FADC-83A and/or FADC-83B and/or | —H | —CH₃ |
| | FADC-83C | | |
| ADC-84 | FADC-84A and/or FADC-84B and/or FADC-84C | 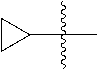 [SMILES: *C1CC1] | —H |
| ADC-85 | FADC-85A and/or FADC-85B and/or FADC-85C | —H | 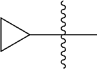 [SMILES: *C1CC1] |

Using LD-81 to LD-84 instead of LD-80, respectively, similar preparation methods were applied to obtain the corresponding ADC-82 to ADC-85, which are Examples 4-82 to 4-85.

### Example 4-86: ADC-86

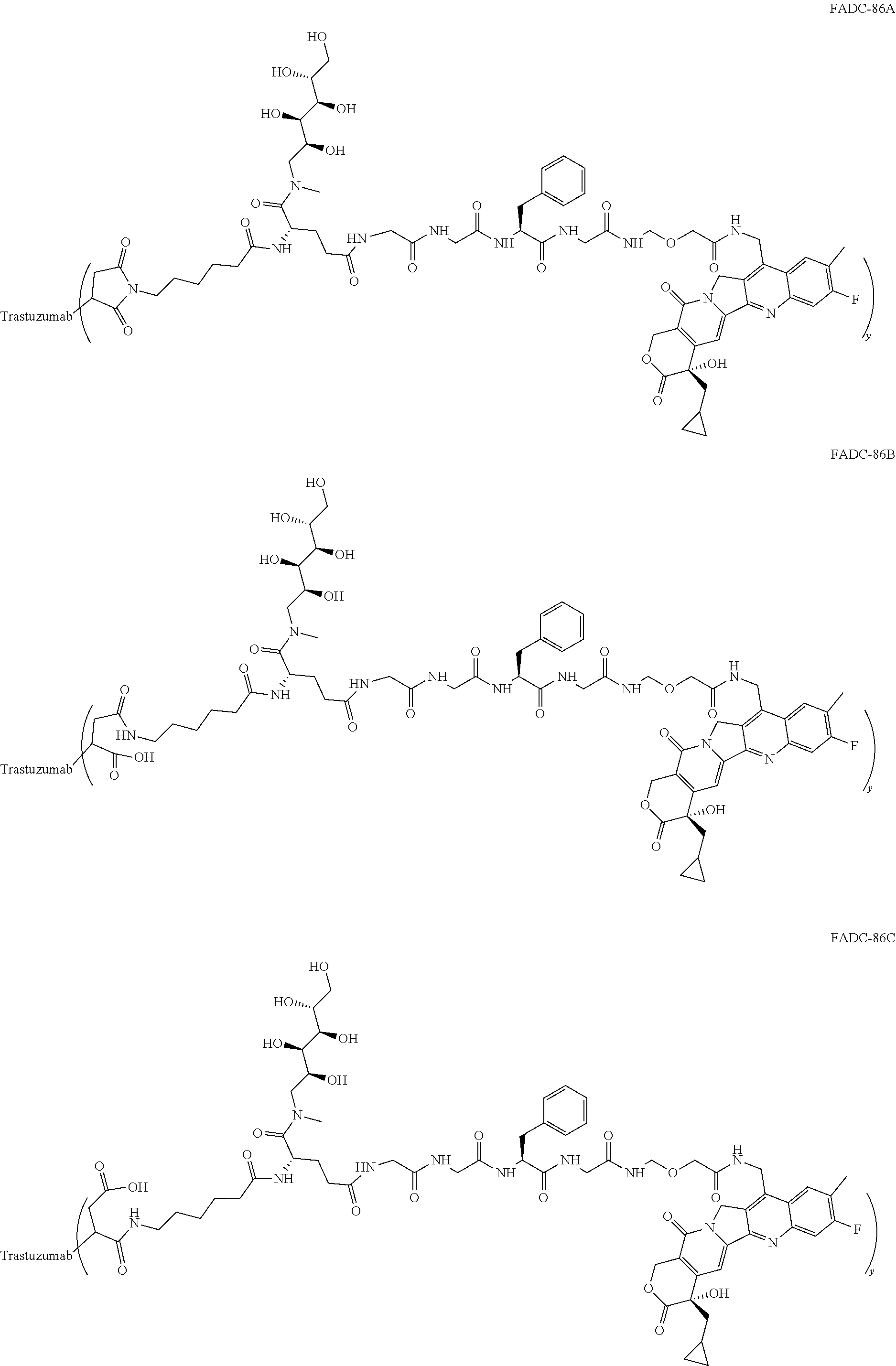

Using the antibody Trastuzumab and LD-85 as starting materials, the preparation was carried out by applying the method disclosed in the patent application “WO2022068898A1”. The PBS buffer with the exemplary product ADC-86 of the conjugate mixture (mixture of FADC-86A and/or FADC-86B and/or FADC-86C) was obtained and stored at 4° C.

### Example 4-87: ADC-87 to Example 4-90: ADC-90

| | |
|---|---|
| 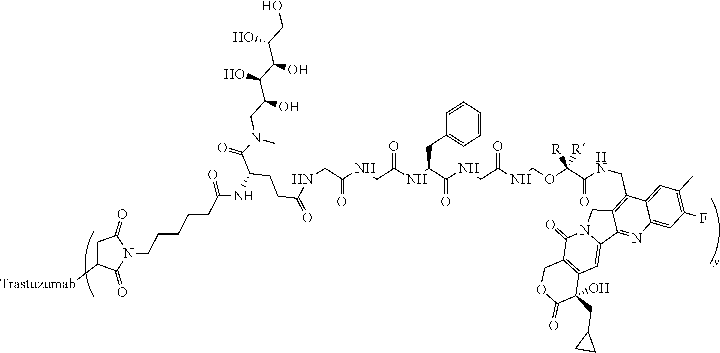 | FADC-X₁₉A |
| 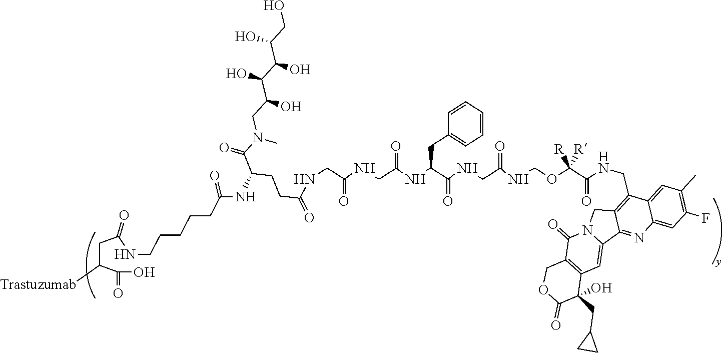 | FADC-X₁₉B |
| 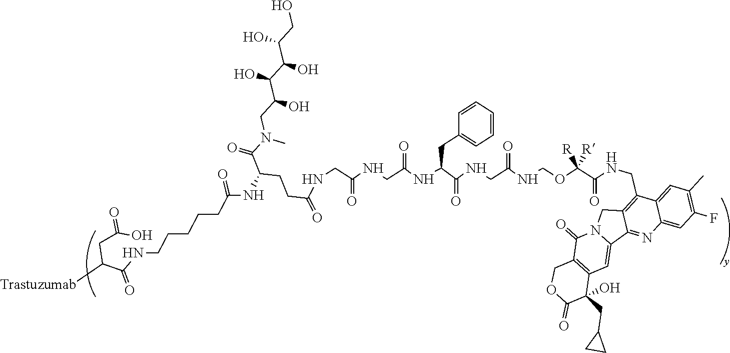 | FADC-X₁₉C |

| Exemplary ADC | Conjugate mixture of FADC-X₁₉A and/or | | |
|---|---|---|---|
| product | FADC-X₁₉B and/or FADC-X₁₉C | R | R′ |
| ADC-87 | FADC-87A and/or FADC-87B and/or | —CH₃ | —H |
| | FADC-87C | | |
| ADC-88 | FADC-88A and/or FADC-88B and/or | —H | —CH₃ |
| | FADC-88C | | |
| ADC-89 | FADC-89A and/or FADC-89B and/or FADC-89C | 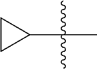 [SMILES: *C1CC1] | —H |
| ADC-90 | FADC-90A and/or FADC-90B and/or FADC-90C | —H | 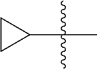 [SMILES: *C1CC1] |

LD-86 to LD-89 were used instead of LD-85, respectively, and similar preparation methods were used to obtain the corresponding ADC-87 to ADC-90, which were Examples 4-87 to 4-90.

### Example 4-91: ADC-91 to Example 4-97: ADC-97

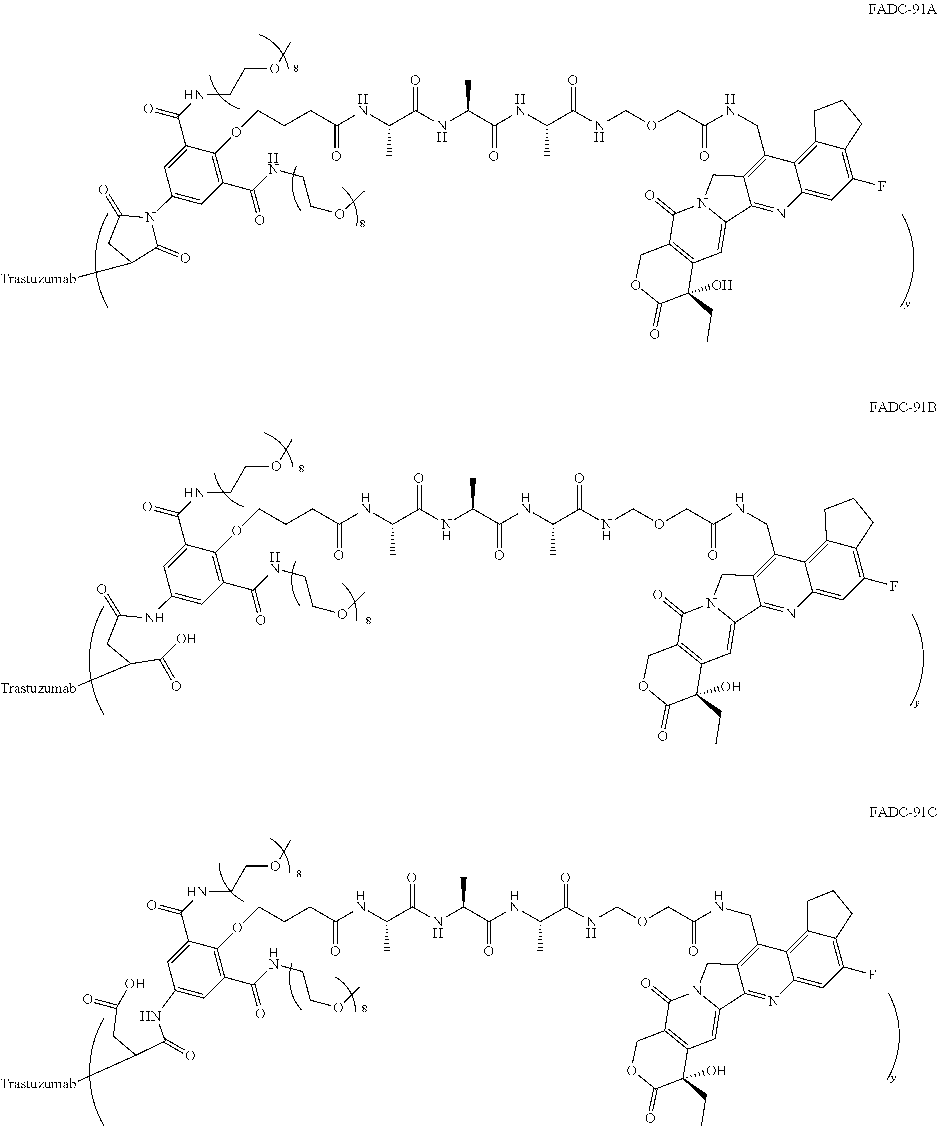

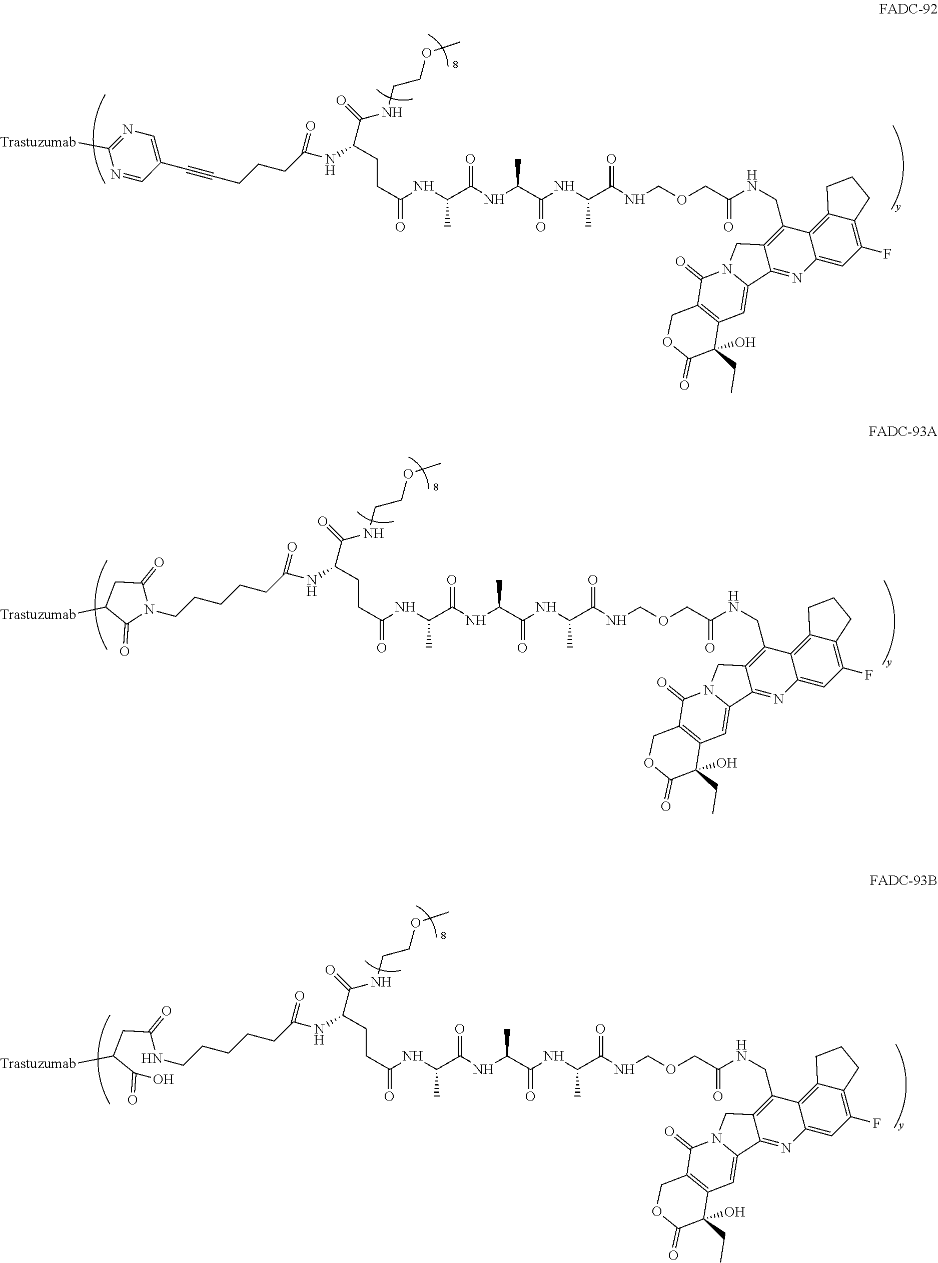

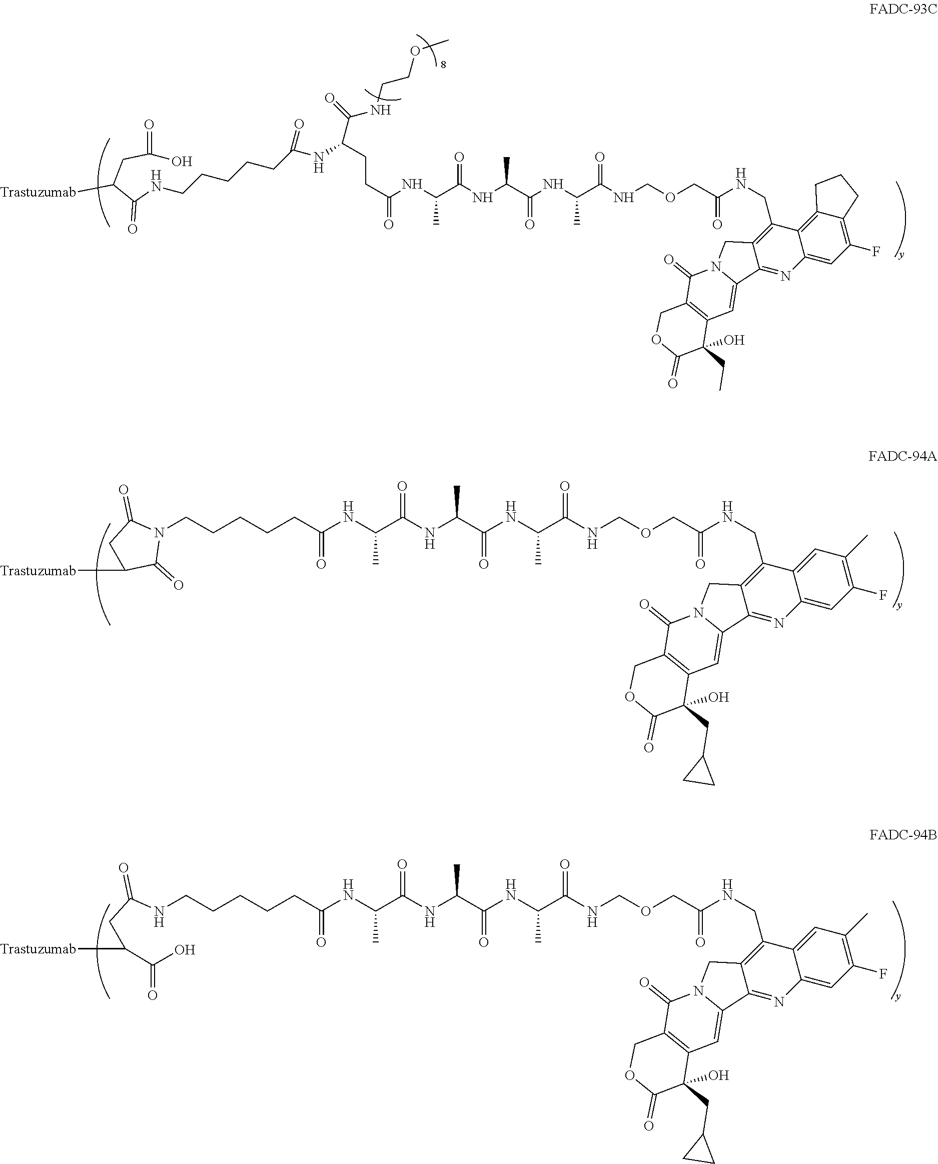

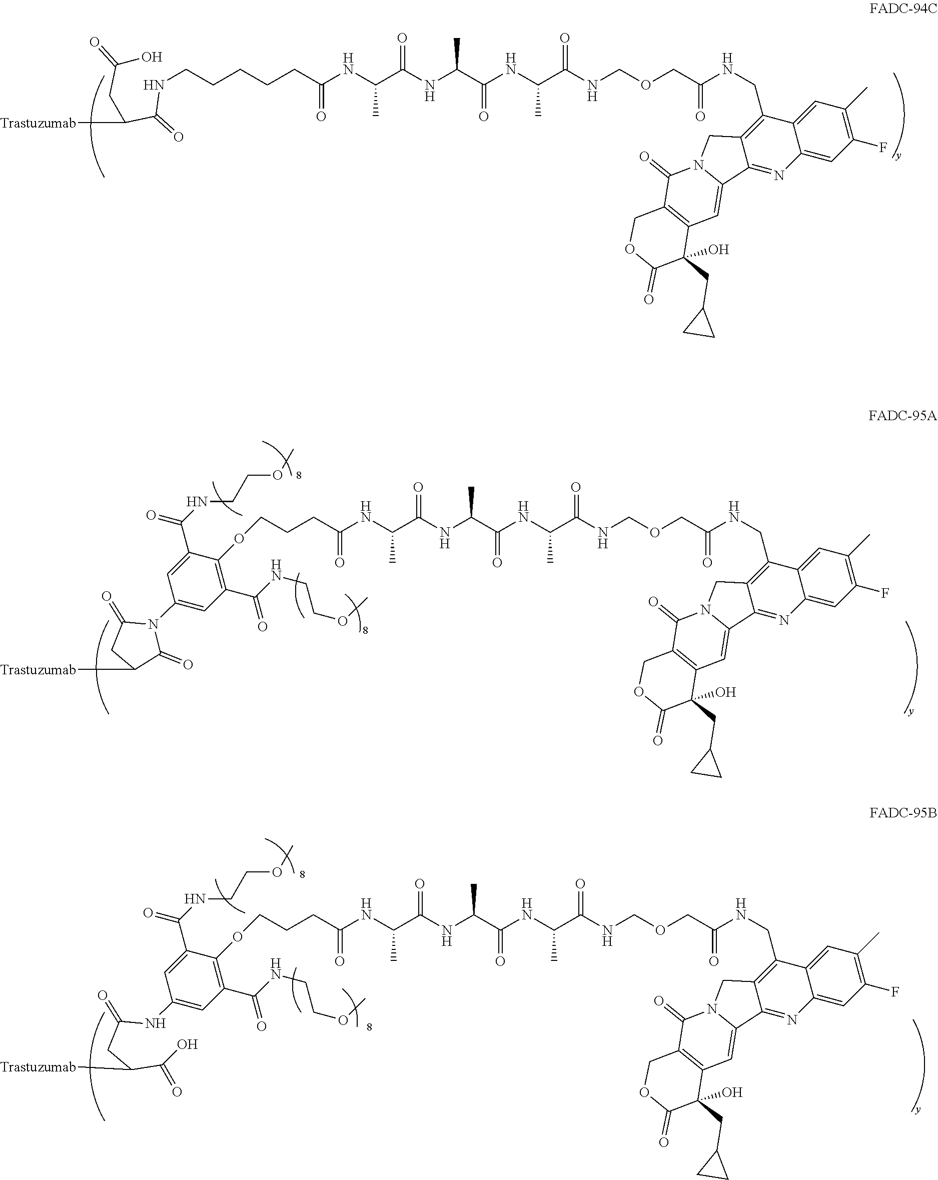

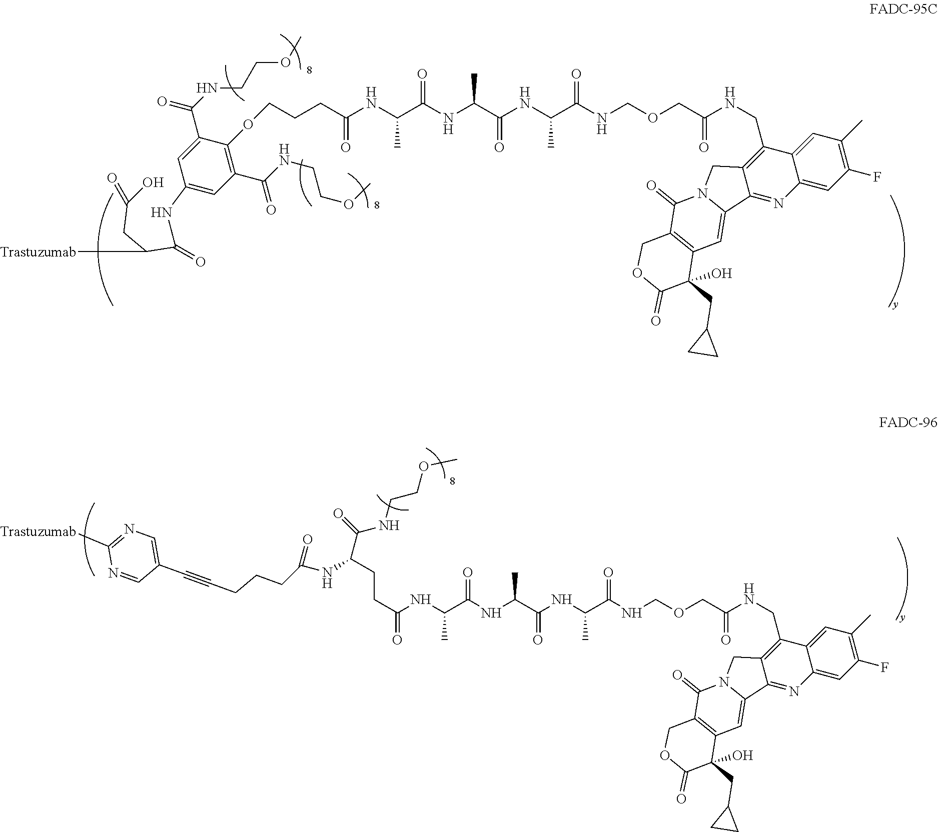

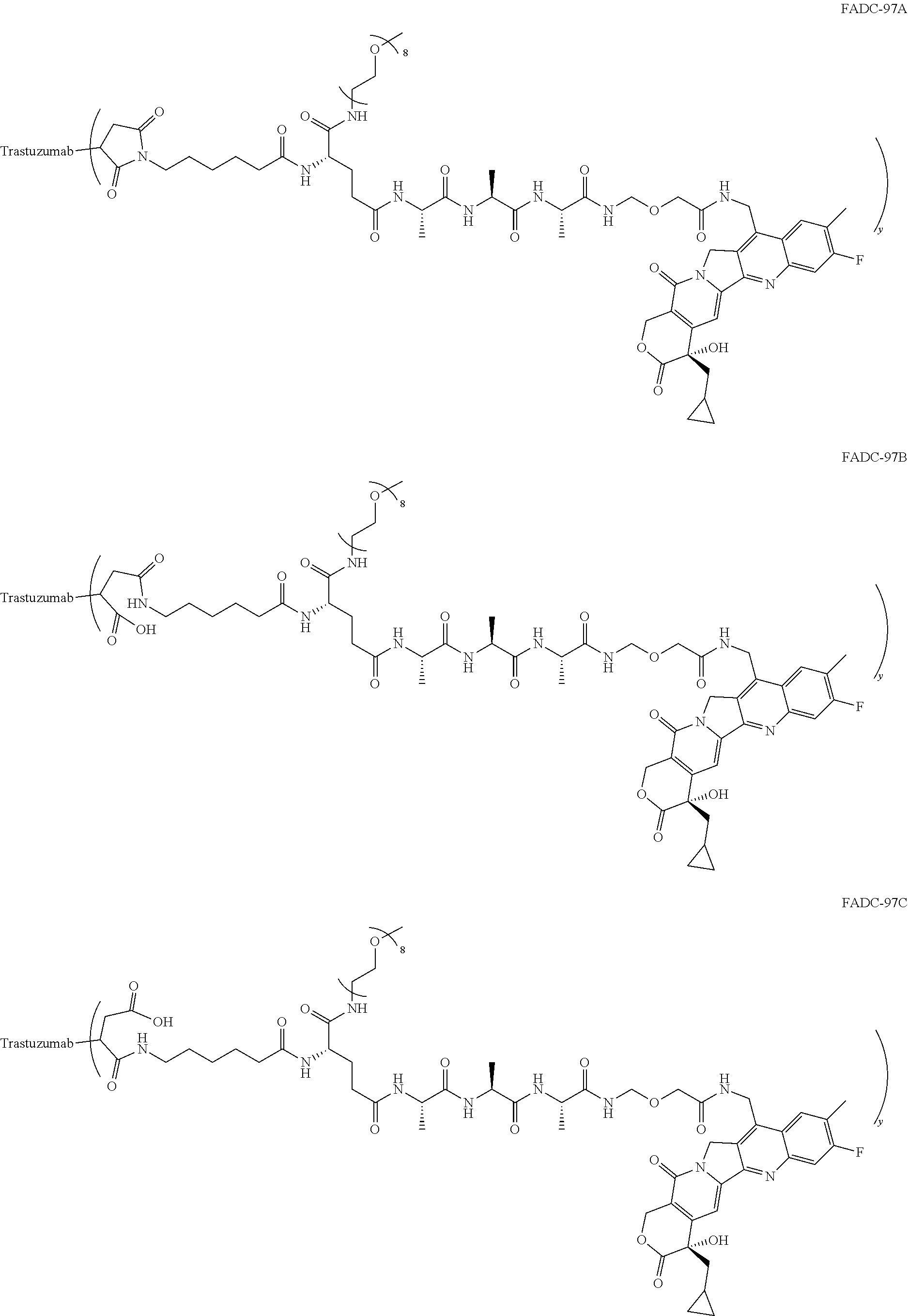

LD-90 to LD-96 and antibody Trastuzumab were used as starting materials, respectively, and similar preparation methods were used to obtain the corresponding ADC-91 to ADC-97, which were Examples 4-91 to 4-97.

### Example 4-98: ADC-98 to Example 4-100: ADC-100

| | |
|---|---|
| 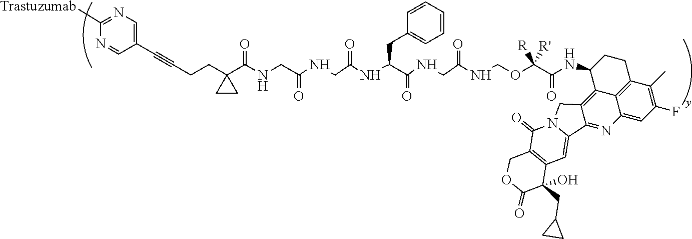 | FADC-X₂₀ |

| | General formula FADC-X₂₀ | |
|---|---|---|
| Exemplary ADC product | R | R′ |
| ADC-98 | —H | —H |
| ADC-99 | —CH₃ | —H |
| ADC-100 | —H | —CH₃ |

LD-97 to LD-99 and antibody Trastuzumab were used as starting materials, respectively, and the preparation was carried out by applying the method of step C. The PBS buffers with the exemplary products ADC-98 to ADC-100 of the corresponding conjugate mixtures FADC-98 to FADC-100 were obtained and stored at 4° C., which were Examples 4-98 to 4-100.

The average drug loading values were calculated by mass spectrometry:
ADC-98, y=7.97;
ADC-99, y=8.00;
ADC-100, y=8.02.
Example 4-101: ADC-101 to Example 4-103: ADC-103

| | |
|---|---|
| 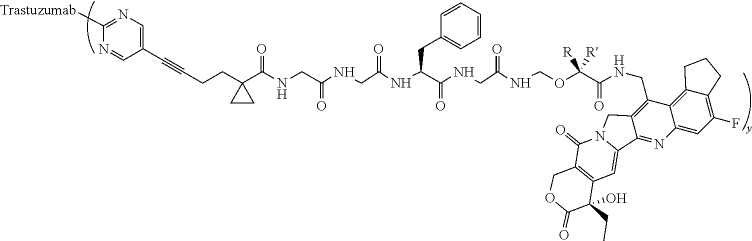 | FADC-X₂₁ |

| | General formula FADC-X₂₁ | |
|---|---|---|
| Exemplary ADC product | R | R′ |
| ADC-101 | —H | —H |
| ADC-102 | —CH₃ | —H |
| ADC-103 | —H | —CH₃ |

LD-100 to LD-102 and antibody Trastuzumab were used as starting materials, respectively, and the preparation was carried out by applying the method disclosed in patent application “WO2019114666A1”. The corresponding ADC-101 to ADC-103 were obtained, which were Examples 4-101 to 4-103.

### Example 4-104: ADC-104

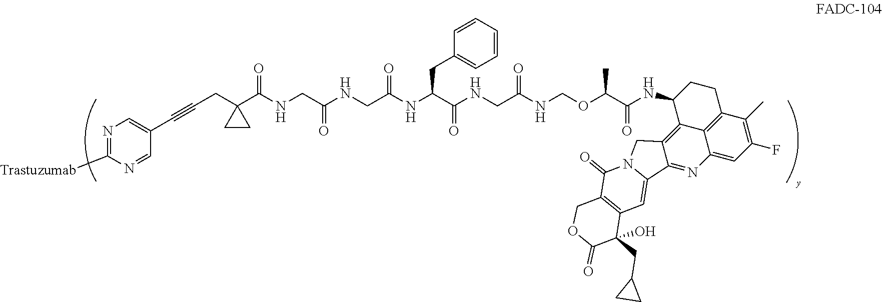

LD-103 and antibody Trastuzumab were used as starting materials, and the preparation was carried out by applying the method of step C. The PBS buffer with the exemplary product ADC-104 of the corresponding conjugate mixture FADC-104 was obtained and stored at 4° C., which was Example 4-104.

The average drug loading value was calculated by mass spectrometry: y=5.85.

### Example 4-105: ADC-105 to Example 4-106: ADC-106

| | |
|---|---|
| 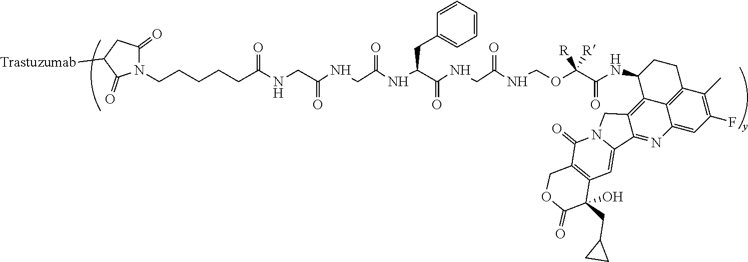 | FADC-X₂₂A |
| 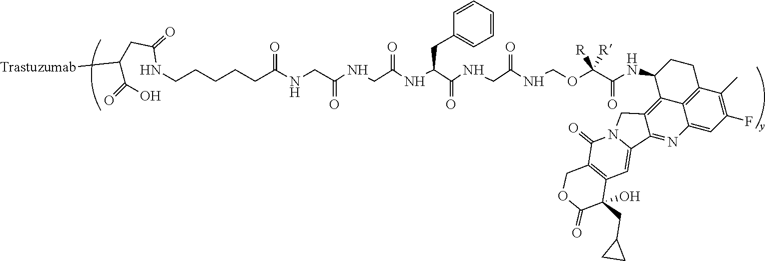 | FADC-X₂₂B |
| 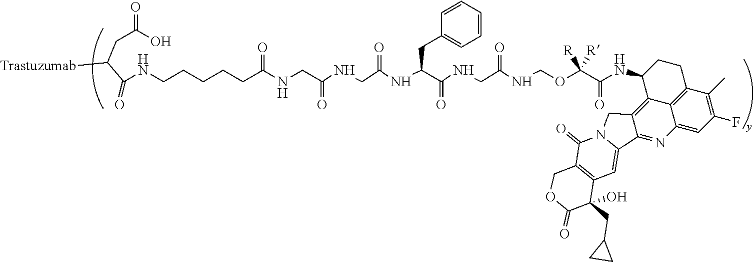 | FADC-X₂₂C |

| Exemplary ADC | Conjugate mixture of FADC-X₂₂A | | |
|---|---|---|---|
| product | and/or FADC-X₂₂B and/or FADC-X₂₂C | R | R′ |
| ADC-105 | FADC-105A and/or FADC-105B and/or | —CH₃ | —H |
| | FADC-105C | | |
| ADC-106 | FADC-106A and/or FADC-106B and/or | —H | —CH₃ |
| | FADC-106C | | |

Using the antibody Trastuzumab and LD-104 as starting materials, the preparation was carried out by applying the method of step B. The PBS buffer with the exemplary product ADC-105 of the conjugate mixture (mixture of FADC-105A and/or FADC-105B and/or FADC-105C) was obtained and stored at 4° C.

The average drug loading value calculated by mass spectrometry: ADC-105, y=8.04.

Using the antibody Trastuzumab and LD-105 as starting materials, the preparation was carried out by applying the method of step B. The PBS buffer with the exemplary product ADC-106 of the conjugate mixture (mixture of FADC-106A and/or FADC-106B and/or FADC-106C) was obtained and stored at 4° C.

The average drug loading value was calculated by mass spectrometry: ADC-106, y=7.95.

### Example 4-107: ADC-107 to Example 4-110: ADC-110

| | |
|---|---|
| 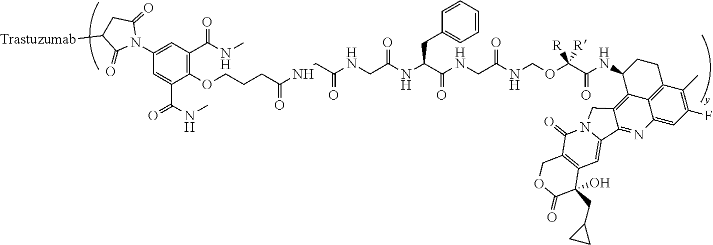 | FADC-X₂₃A |
| 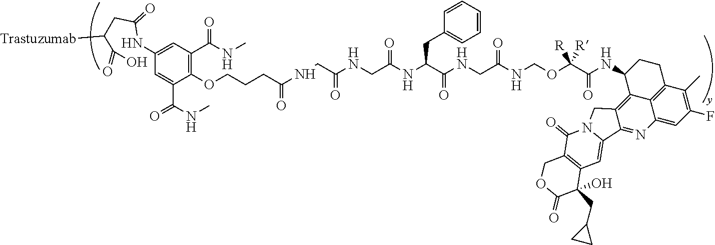 | FADC-X₂₃B |
| 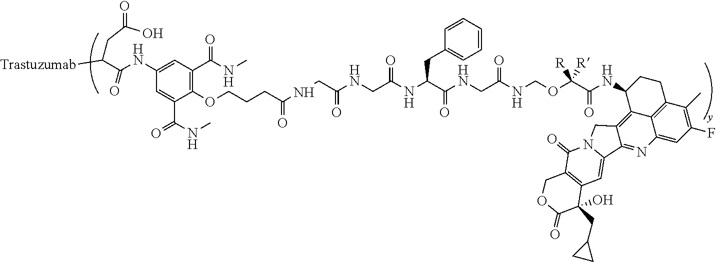 | FADC-X₂₃C |

| Exemplary ADC | Conjugate mixture of FADC-X₂₃A | | |
|---|---|---|---|
| product | and/or FADC-X₂₃B and/or FADC-X₂₃C | R | R′ |
| ADC-107 | FADC-107A and/or FADC-107B | —H | —H |
| | and/or FADC-107C | | |
| ADC-108 | FADC-108A and/or FADC-108B | —CH₃ | —H |
| | and/or FADC-108C | | |
| ADC-109 | FADC-109A and/or FADC-109B and/or FADC-109C | 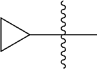 [SMILES: *C1CC1] | —H |
| ADC-110 | FADC-110A and/or FADC-110B and/or FADC-110C | —H | 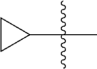 [SMILES: *C1CC1] |

Using the antibody Trastuzumab and LD-106 as starting materials, the preparation was carried out by applying the method of step A-1. The PBS buffer with the exemplary product ADC-107 of the conjugate mixture (mixture of FADC-107A and/or FADC-107B and/or FADC-107C) was obtained and stored at 4° C., which was Example 4-107.

Using LD-107 to LD-109 and antibody Trastuzumab as starting materials, respectively, the preparation was carried out by applying the method of step A-1, and the corresponding PBS buffers of ADC-108 to ADC110 were obtained and stored at 4° C., which were Examples 4-108 to 4-110.

The average drug loading values were calculated by mass spectrometry:
ADC-107, y=8.09;
ADC-108, y=7.43;
ADC-109, y=7.80;
ADC-110, y=7.66.

### Example 4-111: ADC-111

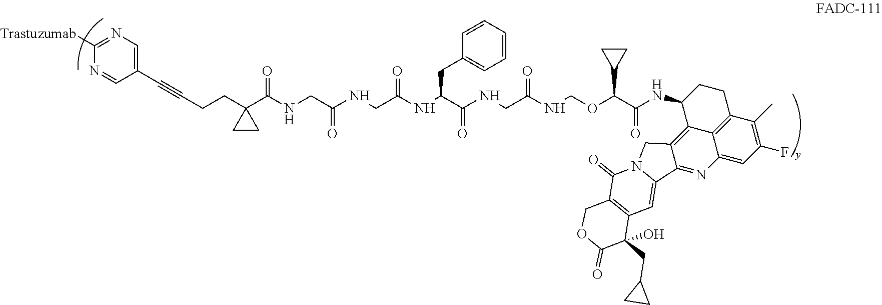

Using the antibody Trastuzumab and LD-110 as starting materials, the preparation was carried out by applying the method of step C. The PBS buffer with the exemplary product ADC-111 of the conjugate mixture FADC-111 was obtained and stored at 4° C., which was Example 4-111.

The average drug loading value was calculated by mass spectrometry:
ADC-111, y=7.74;

### Example 4-112: ADC-112

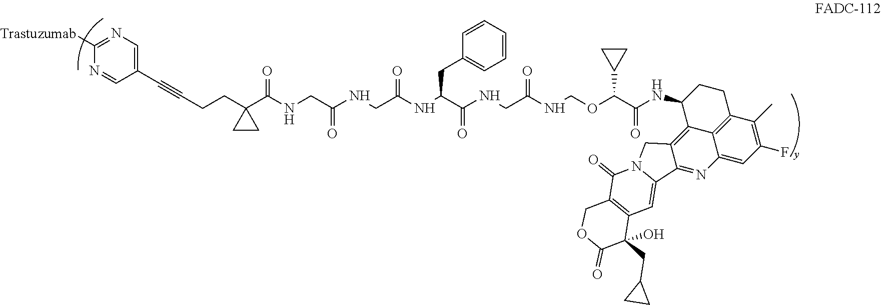

Using the antibody Trastuzumab and LD-111 as starting materials, the preparation was carried out by applying the method of step C, and the PBS buffer with the exemplary product ADC-112 of the conjugate mixture FADC-112 was obtained and stored at 4° C., which was Example 4-112.

The average drug loading value was calculated by mass spectrometry:
ADC-112, y=7.86.

### Example 4-113: ADC-113 to Example 4-117: ADC-117

| | |
|---|---|
| 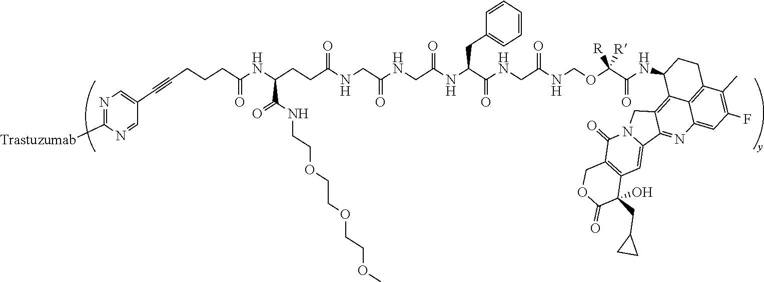 | FADC-X₂₄ |

| | General formula FADC-X₂₄ | |
|---|---|---|
| Exemplary ADC product | R | R′ |
| ADC-113 | —H | —H |
| ADC-114 | —CH₃ | —H |
| ADC-115 | —H | —CH₃ |
| ADC-116 | 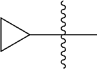 [SMILES: *C1CC1] | —H |
| ADC-117 | —H | 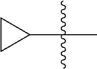 [SMILES: *C1CC1] |

Using the antibody Trastuzumab and LD-112 as starting materials, the preparation was carried out by applying the method of step C. The PBS buffer with the exemplary product ADC-113 of the conjugate mixture FADC-113 was obtained and stored at 4° C., which was Example 4-113.

Using LD-113 to LD-116 and the antibody Trastuzumab as starting materials, respectively, the preparation was carried out by similar preparation methods, and the corresponding PBS buffers of ADC-114 to ADC117 were obtained and stored at 4° C., respectively, which were Examples 4-114 to 4-117.

The average drug loading values were calculated by mass spectrometry:
ADC-113, y=8.02;
ADC-114, y=7.91;
ADC-115, y=6.93;
ADC-116, y=7.88;
ADC-117, y=7.99.

### Example 4-118: Reference 1

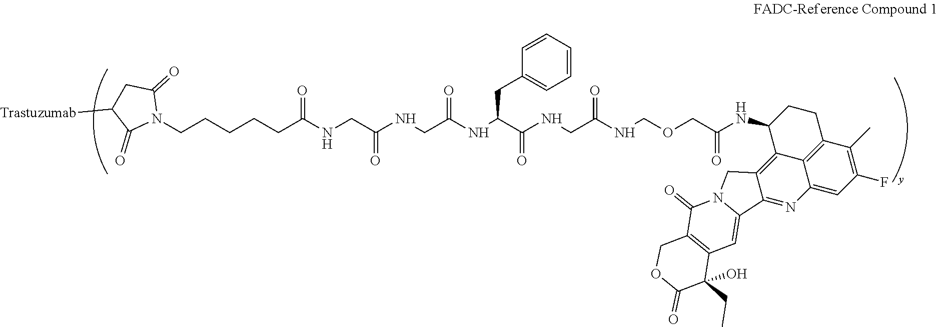

Using the antibody Trastuzumab and N-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2,5-dioxo-2,5-dihydro-1H-pyrrol-1-yl)hexanamide as starting materials, the preparation was carried out by applying the method of step B. The PBS buffer with the exemplary product Reference 1 of the conjugate mixture FADC-Reference 1 was obtained and stored at 4° C., which was Example 4-118.

The average drug loading value was calculated by mass spectrometry: y=8.00.

### Example 4-119: Reference 2

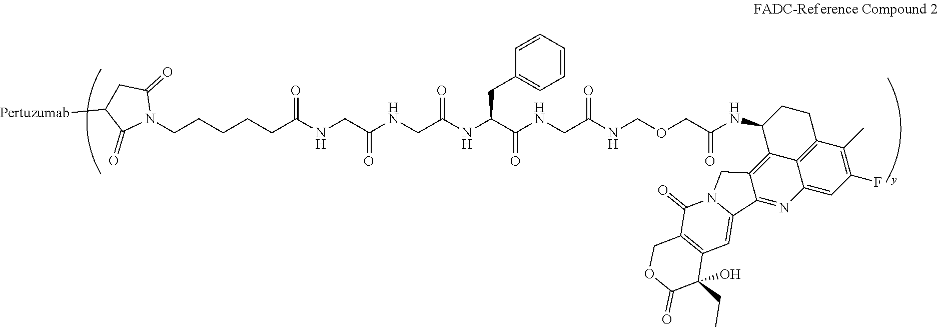

Using the antibody Pertuzumab and N-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2,5-dioxo-2,5-dihydro-1H-pyrrol-1-yl)hexanamide as starting materials, the preparation was carried out by applying the method of step B. The PBS buffer of the exemplary product Reference 2 of the conjugate mixture FADC-Reference 2 was obtained and stored at 4° C., which was Example 4-119.

The average drug loading value was calculated by mass spectrometry: y=7.67.

### Example 4-120: ADC-118

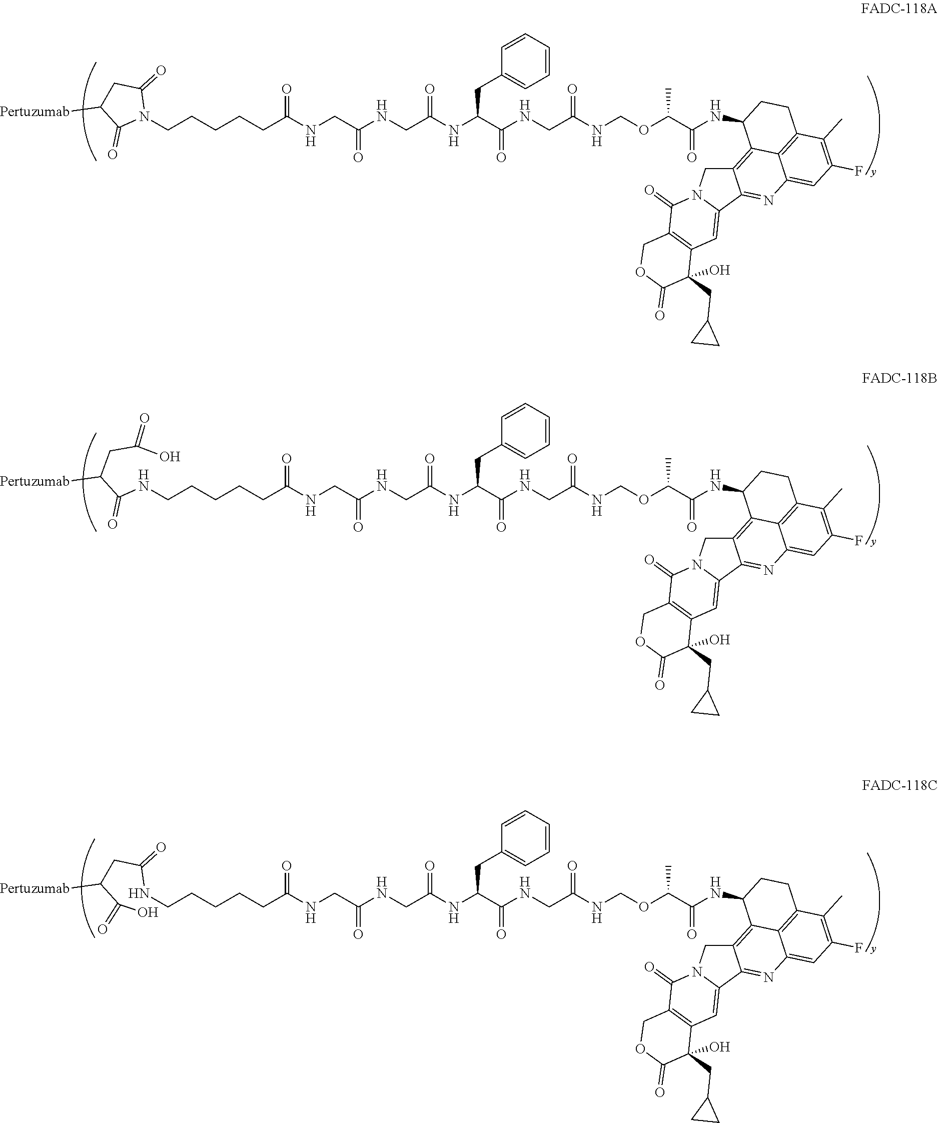

Using the antibody Pertuzumab and LD-104 as starting materials, the preparation was carried out by applying the method of step B. The PBS buffer with the exemplary product ADC-118 of the conjugate mixture (mixture of FADC-118A and/or FADC-118B and/or FADC-118C) was obtained and stored at 4° C., which was Example 4-120.

The average drug loading value was calculated by mass spectrometry: y=4.95.

### Example 4-121: ADC-119

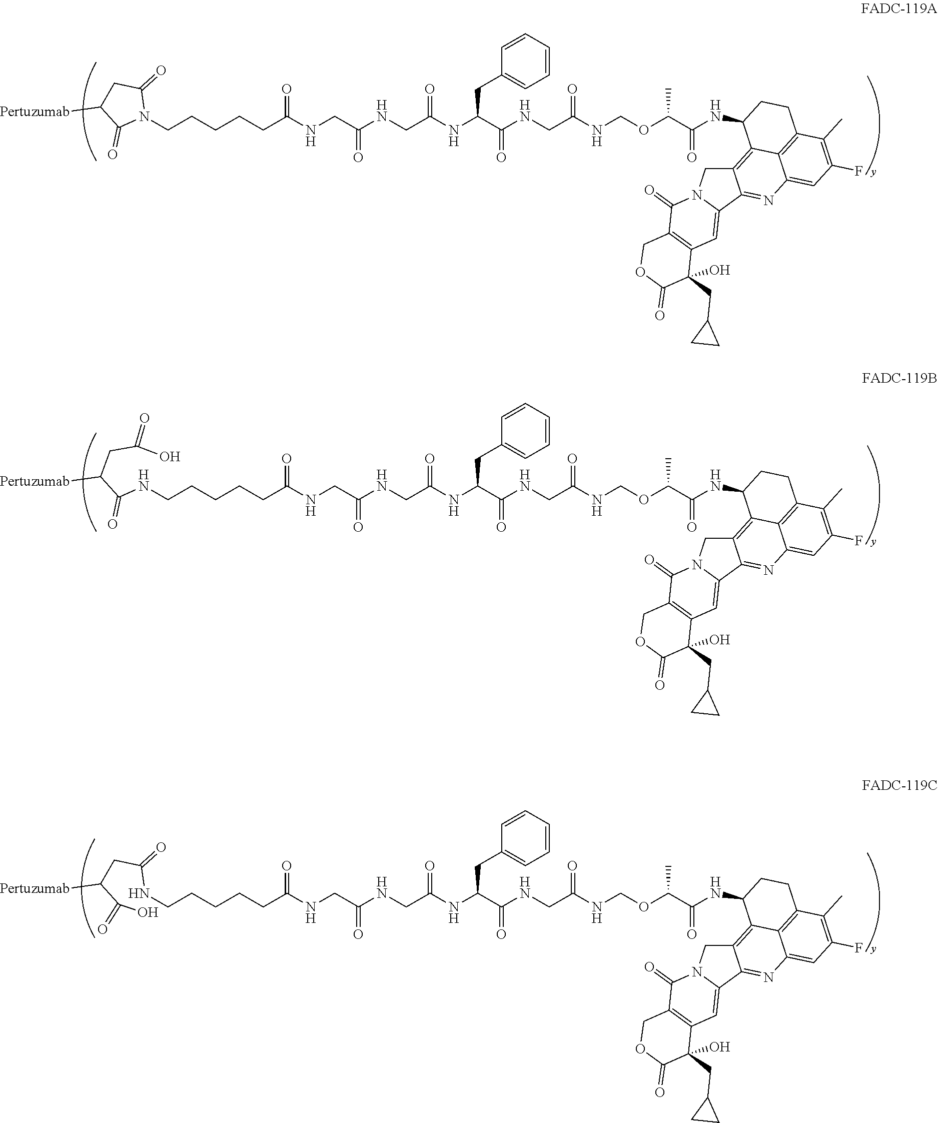

Using the antibody Pertuzumab and LD-105 as starting materials, the preparation was carried out by applying the method of step B. The PBS buffer with the exemplary product ADC-119 of the conjugate mixture (mixture of FADC-119A and/or FADC-119B and/or FADC-119C) was obtained and stored at 4° C., which was Example 4-121.

The average drug loading value was calculated by mass spectrometry: y=3.06.

### Example 4-122: ADC-120

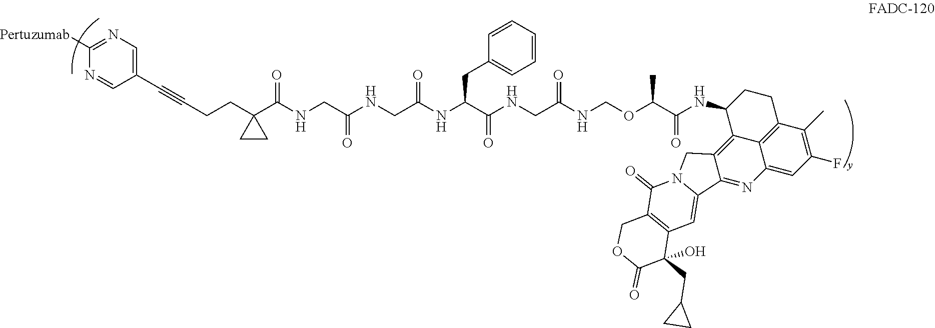

Using the antibody Pertuzumab and LD-98 as starting materials, the preparation was carried out by applying the method of step C. The PBS buffer with the exemplary product ADC-120 of the conjugate mixture FADC-120 was obtained and stored at 4° C., which was Example 4-122.

The average drug loading value was calculated by mass spectrometry: y=7.48.

### Example 4-123: ADC-121

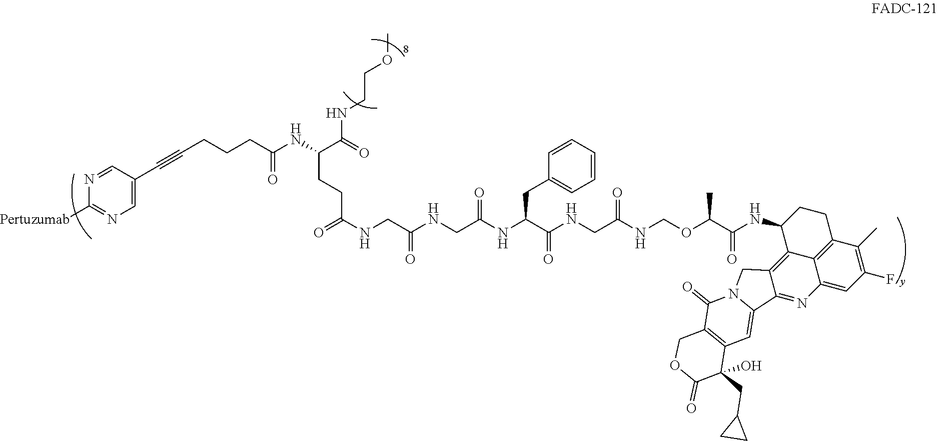

Using the antibody Pertuzumab and LD-13 as starting materials, the preparation was carried out by applying the method of step C. The PBS buffer with the exemplary product ADC-121 of the conjugate mixture FADC-121 was obtained and stored at 4° C., which was Example 4-123.

The average drug loading value was calculated by mass spectrometry: y=7.59.

### Example 4-124: ADC-122

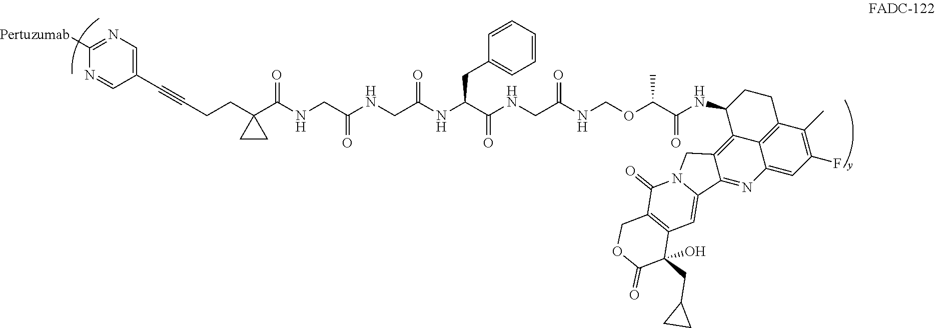

Using the antibody Pertuzumab and LD-99 as starting materials, the preparation was carried out by applying the method of step C. The PBS buffer with the exemplary product ADC-122 of the conjugate mixture FADC-122 was obtained and stored at 4° C., which was Example 4-124.

The average drug loading value was calculated by mass spectrometry: y=7.89.

### Example 4-125: ADC-123

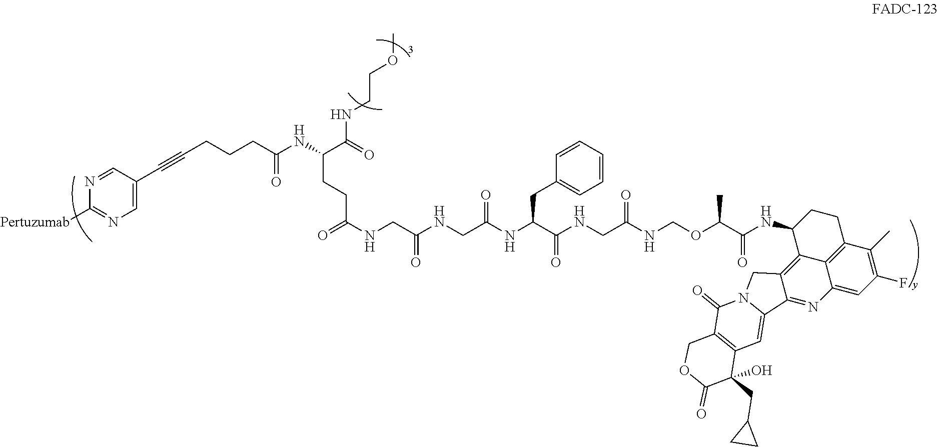

Using the antibody Pertuzumab and LD-113 as starting materials, the preparation was carried out by applying the method of step C. The PBS buffer with the exemplary product ADC-123 of the conjugate mixture FADC-123 was obtained and stored at 4° C., which was Example 4-125.

The average drug loading value was calculated by mass spectrometry: y=7.99.

### Example 4-126: ADC-124

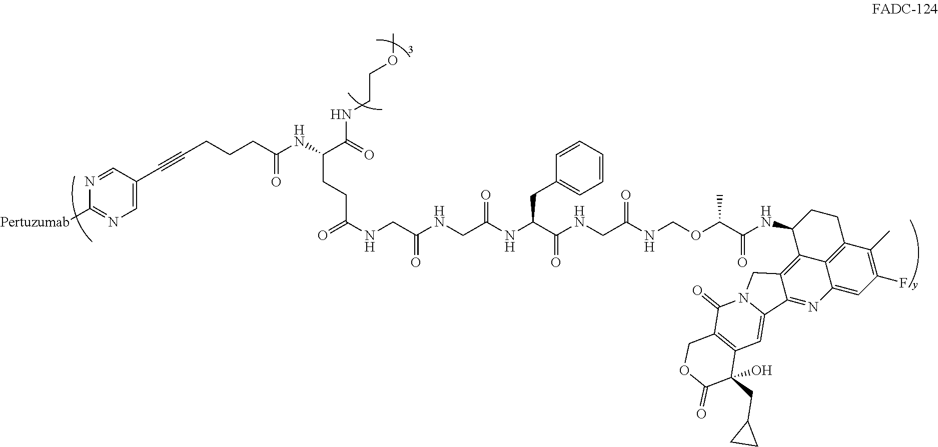

Using the antibody Pertuzumab and LD-114 as starting materials, the preparation was carried out by applying the method of step C. The PBS buffer with the exemplary product ADC-124 of the conjugate mixture FADC-124 was obtained and stored at 4° C., which was Example 4-126.

The average drug loading value was calculated by mass spectrometry: y=7.82.

### Example 4-127: ADC-125

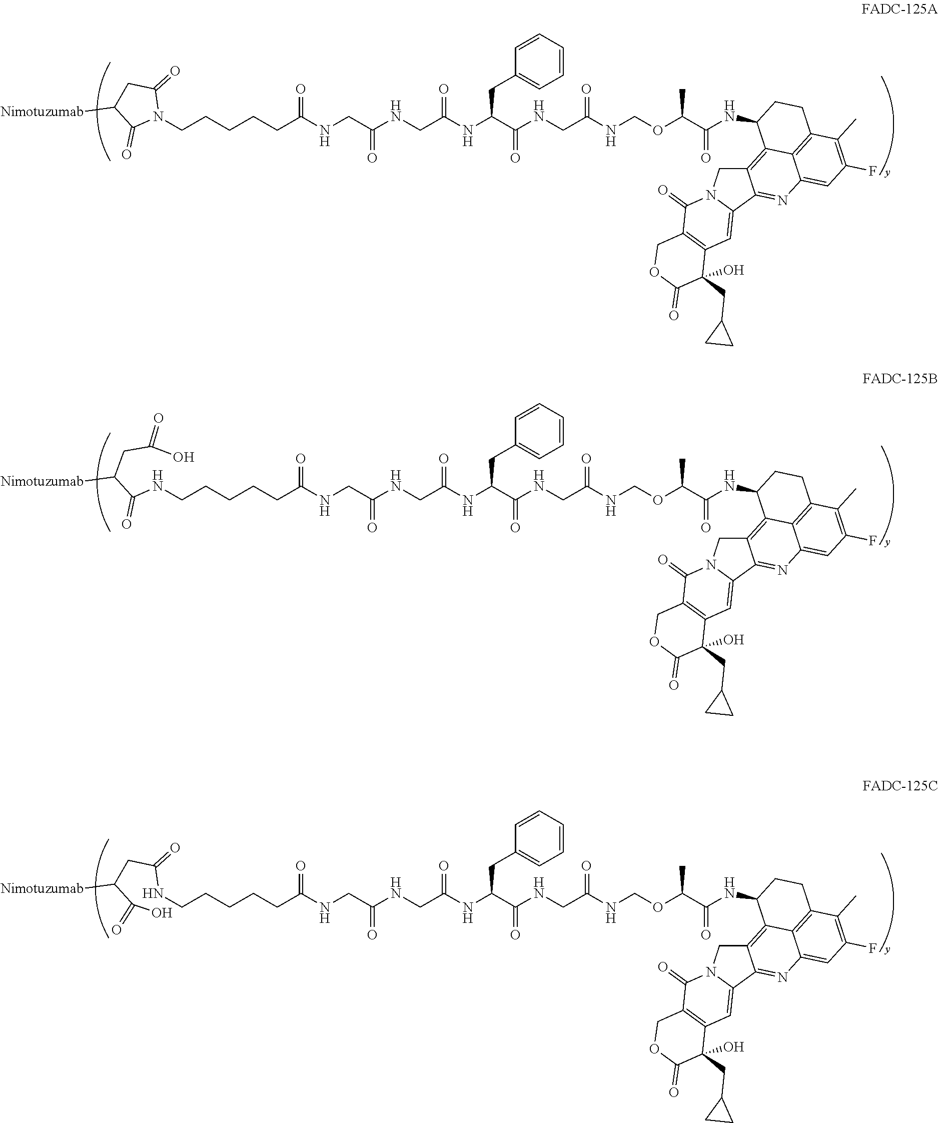

Using the antibody Nimotuzumab and LD-104 as starting materials, the preparation was carried out by applying the method of step B. The PBS buffer with the exemplary product ADC-125 of the conjugate mixture (mixture of FADC-125A and/or FADC-125B and/or FADC-125C) was obtained and stored at 4° C., which was Example 4-127.

The average drug loading value was calculated by mass spectrometry: y=5.66.

### Example 4-128: ADC-126

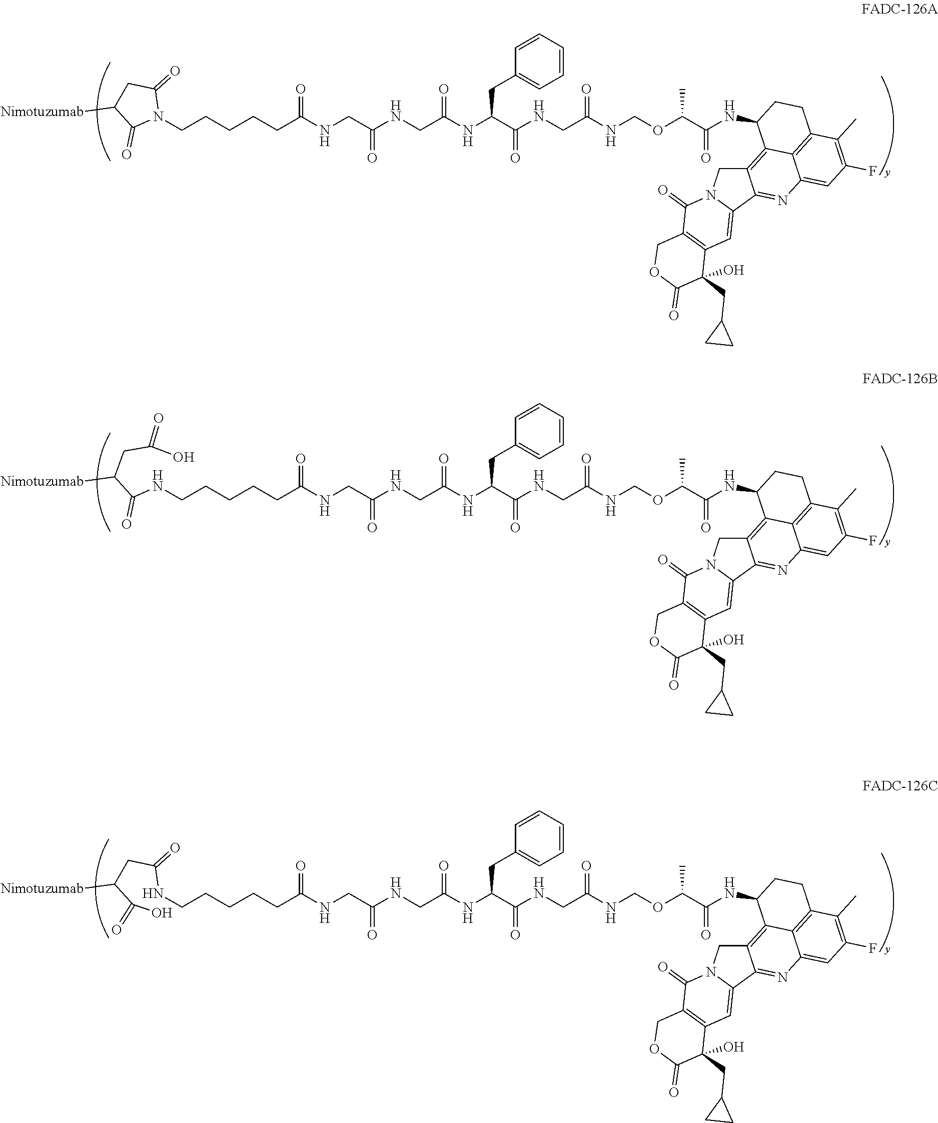

Using the antibody Nimotuzumab and LD-105 as starting materials, the preparation was carried out by applying the method of step B. The PBS buffer with the exemplary product ADC-126 of the conjugate mixture (mixture of FADC-126A and/or FADC-126B and/or FADC-126C) was obtained and stored at 4° C., which was Example 4-128.

The average drug loading value was calculated by mass spectrometry: y=4.96.

### Example 4-129: ADC-127

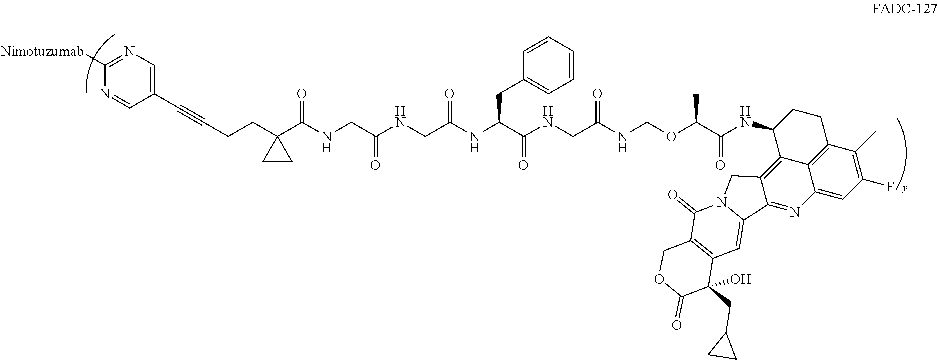

Using the antibody Nimotuzumab and LD-98 as starting materials, the preparation was carried out by applying the method of step C. The PBS buffer with the exemplary product ADC-127 of the conjugate mixture FADC-127 was obtained and stored at 4° C., which was Example 4-129.

The average drug loading value was calculated by mass spectrometry: y=6.31.

### Example 4-130: ADC-128

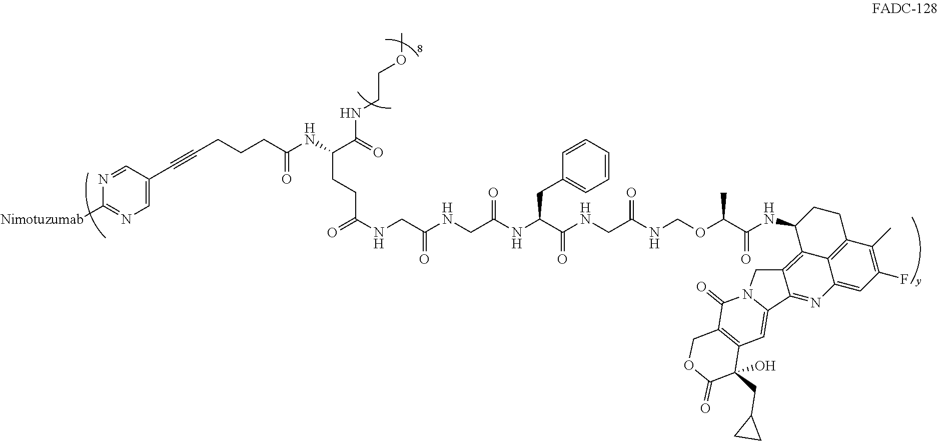

Using the antibody Nimotuzumab and LD-13 as starting materials, the preparation was carried out by applying the method of step C. The PBS buffer with the exemplary product ADC-128 of the conjugate mixture FADC-128 was obtained and stored at 4° C., which was Example 4-130.

The average drug loading value was calculated by mass spectrometry: y=7.62.

### Example 4-131: ADC-129

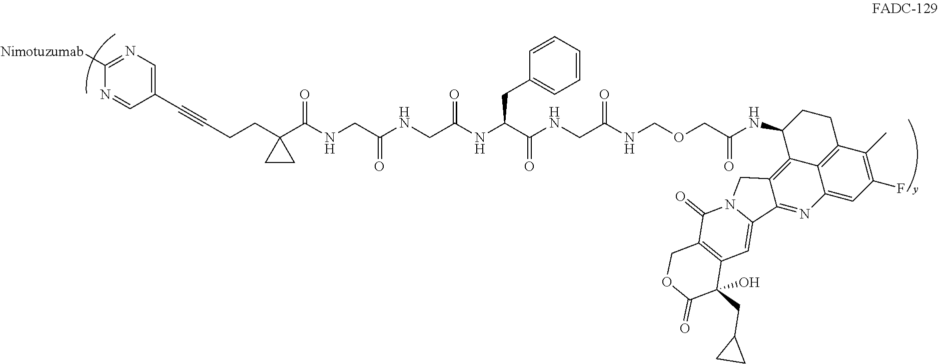

Using the antibody Nimotuzumab and LD-99 as starting materials, the preparation was carried out by applying the method of step C. The PBS buffer with the exemplary product ADC-129 of the conjugate mixture FADC-129 was obtained and stored at 4° C., which was Example 4-131.

The average drug loading value was calculated by mass spectrometry: y=7.11.

### Example 4-132: ADC-130

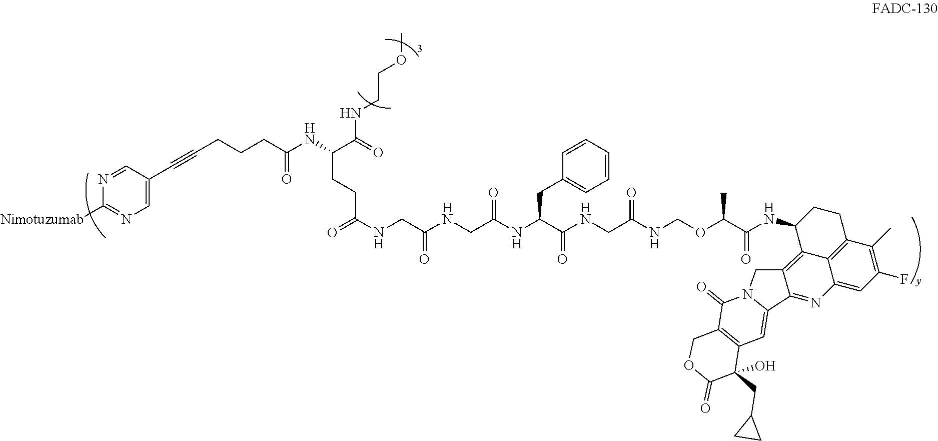

Using the antibody Nimotuzumab and LD-113 as starting materials, the preparation was carried out by applying the method of step C. The PBS buffer with the exemplary product ADC-130 of the conjugate mixture FADC-130 was obtained and stored at 4° C., which was Example 4-132.

The average drug loading value was calculated by mass spectrometry: y=7.18.

### Example 4-133: ADC-131

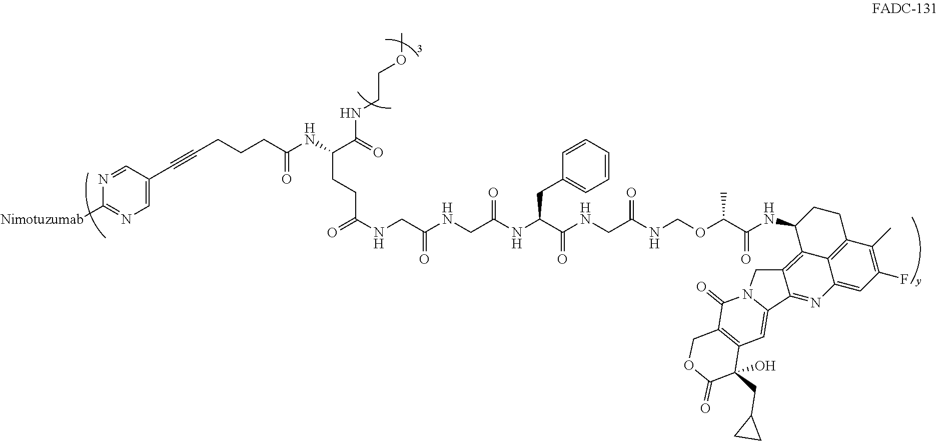

Using the antibody Nimotuzumab and LD-114 as starting materials, the preparation was carried out by applying the method of step C. The PBS buffer with the exemplary product ADC-131 of the conjugate mixture FADC-131 was obtained and stored at 4° C., which was Example 4-133.

The average drug loading value was calculated by mass spectrometry: y=7.30.

### Example 4-134: Reference 3

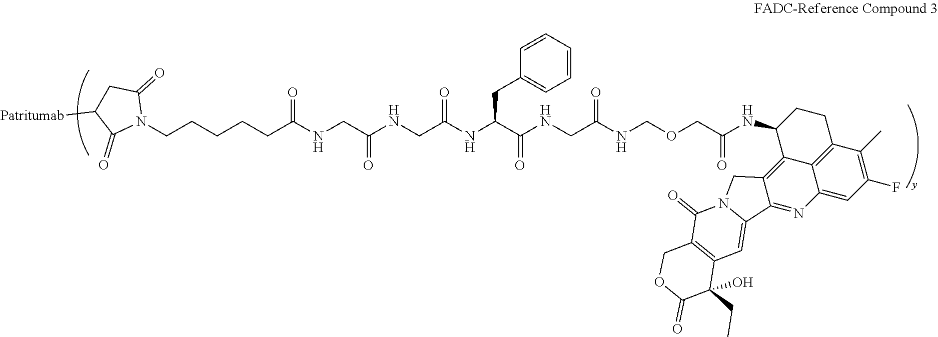

Using the antibody Patritumab and N-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2,5-dioxo-2,5-dihydro-1H-pyrrol-1-yl)hexanamide as starting materials, the preparation was carried out by applying the method of step B. The PBS buffer with the exemplary product Reference 3 of the conjugate mixture FADC-Reference 3 was obtained and stored at 4° C., which was Example 4-134.

The average drug loading value was calculated by mass spectrometry: y=7.59.

### Example 4-135: ADC-132

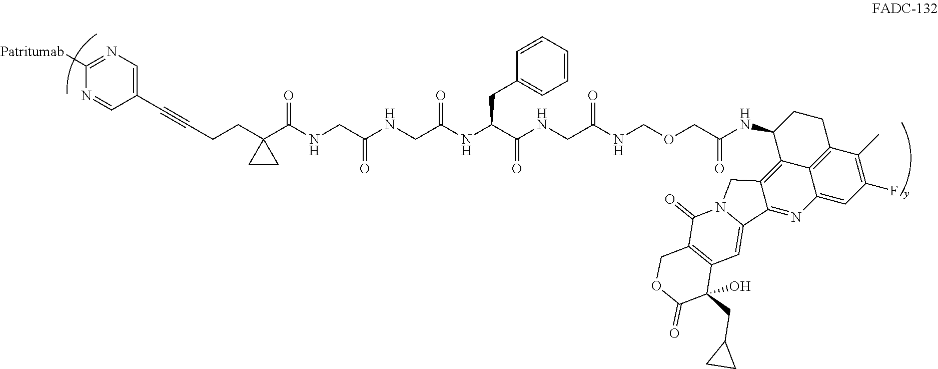

Using the antibody Patritumab and LD-97 as starting materials, the preparation was carried out by applying the method of step C. The PBS buffer with the exemplary product ADC-132 of the conjugate mixture FADC-132 was obtained and stored at 4° C., which was Example 4-135.

The average drug loading value was calculated by mass spectrometry: y=7.73.

### Example 4-136: ADC-133

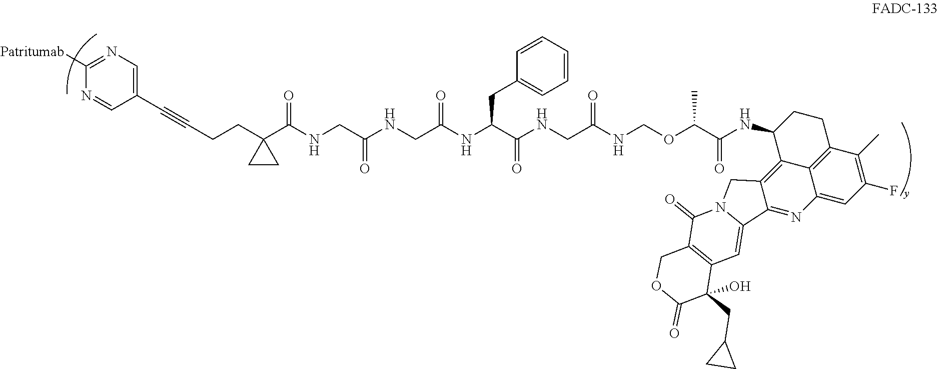

Using the antibody Patritumab and LD-99 were used as starting materials, the preparation was carried out by applying the method of step C. The PBS buffer with the exemplary product ADC-133 of the conjugate mixture FADC-133 was obtained and stored at 4° C., which was Example 4-136.

The average drug loading value was calculated by mass spectrometry: y=7.72.

### Example 4-137: ADC-134

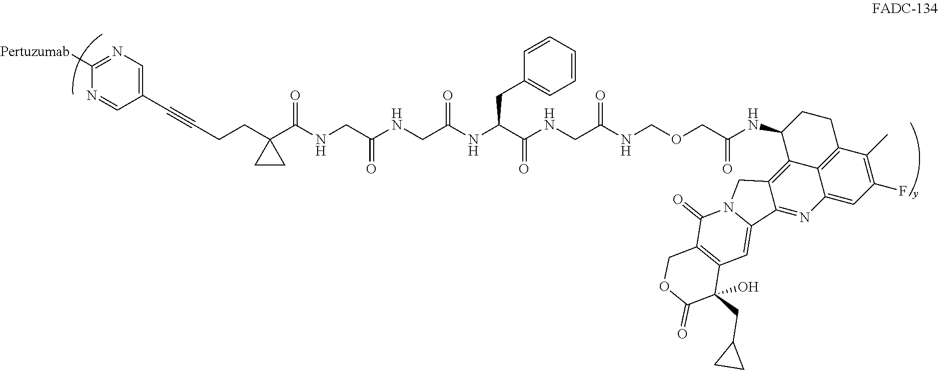

Using the antibody Pertuzumab and LD-97 as starting materials, the preparation was carried out by applying the method of step C. The PBS buffer with the exemplary product ADC-134 of the conjugate mixture FADC-134 was obtained and stored at 4° C., which was Example 4-137.

The average drug loading value was calculated by mass spectrometry: y=7.80.

### Example 4-138: ADC-135

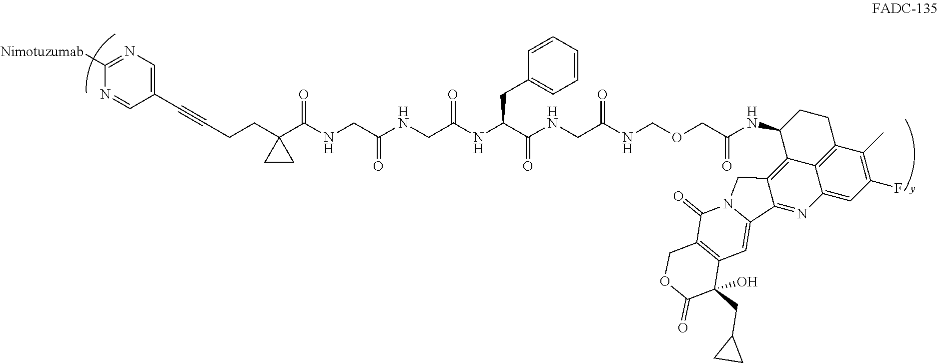

Using the antibody Nimotuzumab and LD-97 as starting materials, the preparation was carried out by applying the method of step C. The PBS buffer with the exemplary product ADC-135 of the conjugate mixture FADC-135 was obtained and stored at 4° C., which was Example 4-138.

The average drug loading value was calculated by mass spectrometry: y=7.02.

### Example 4-139: ADC-136

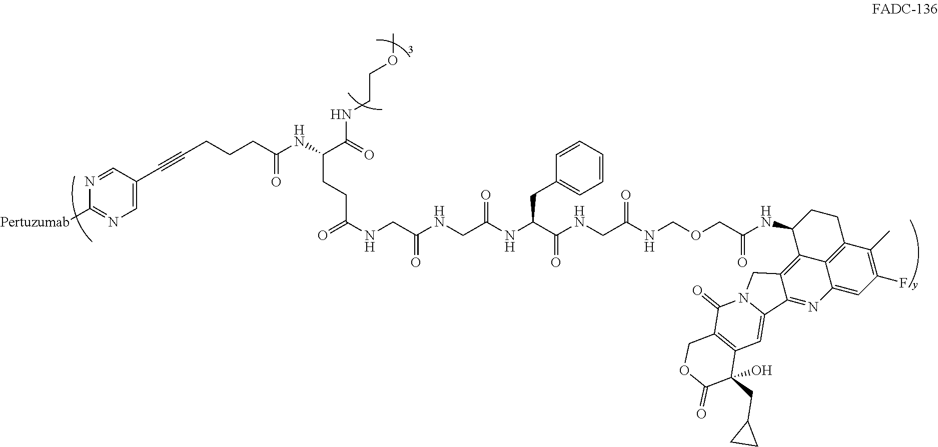

Using the antibody Pertuzumab and LD-112 as starting materials, the preparation was carried out by applying the method of step C. The PBS buffer with the exemplary product ADC-136 of the conjugate mixture FADC-136 was obtained and stored at 4° C., which was Example 4-139.

The average drug loading value was calculated by mass spectrometry: y=7.90.

### Example 4-140: ADC-137

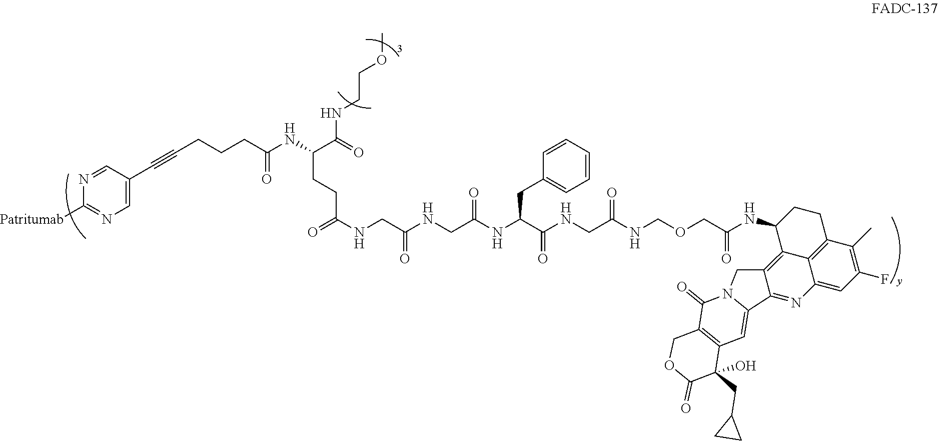

Using the antibody Patritumab and LD-112 as starting materials, the preparation was carried out by applying the method of step C. The PBS buffer with the exemplary product ADC-137 of the conjugate mixture FADC-137 was obtained and stored at 4° C., which was Example 4-140.

The average drug loading value was calculated by mass spectrometry: y=7.67.

### Example 4-141: ADC-138

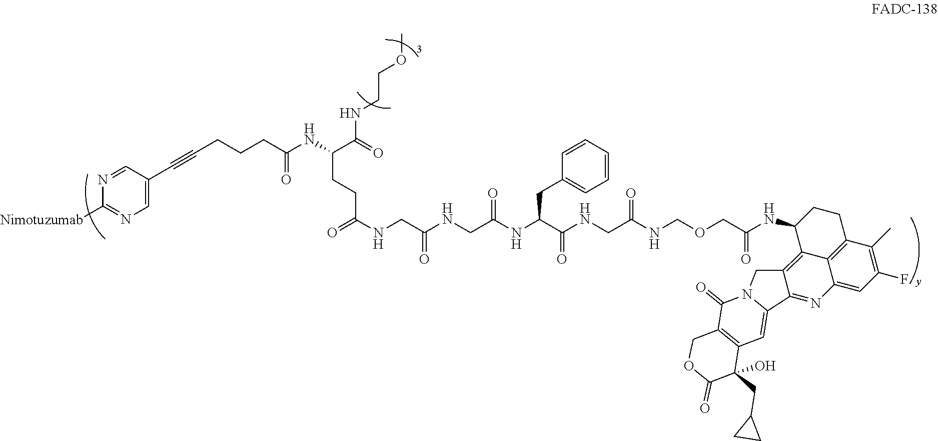

Using the antibody Nimotuzumab and LD-112 as starting materials, the preparation was carried out by applying the method of step C. The PBS buffer with the exemplary product ADC-138 of the conjugate mixture FADC-138 was obtained and stored at 4° C., which was Example 4-141.

The average drug loading value was calculated by mass spectrometry: y=6.84.

### Example 4-142: ADC-139

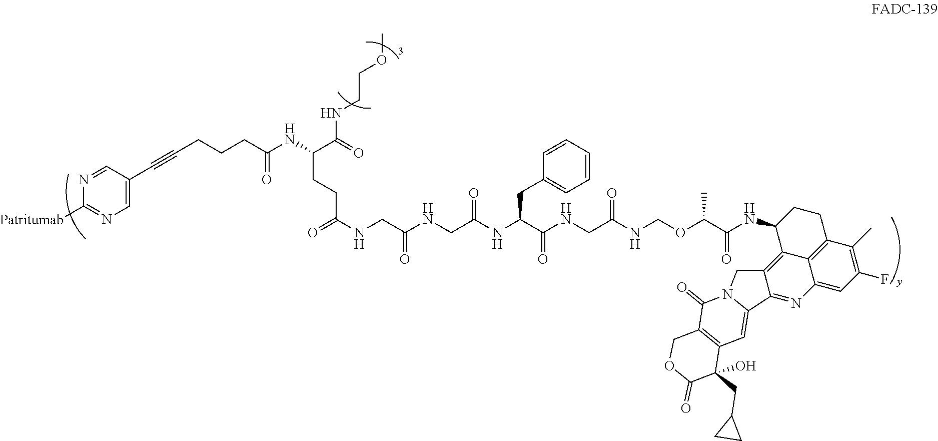

Using the antibody Nimotuzumab and LD-114 as starting materials, the preparation was carried out by applying the method of step C. The PBS buffer with the exemplary product ADC-139 of the conjugate mixture FADC-139 was obtained and stored at 4° C., which was Example 4-142.

The average drug loading value was calculated by mass spectrometry: y=7.73.

Analysis of drug loading of ADC stock solution

### Experimental Objective and Principle

ADC stock solution is an antibody conjugate drug, and its mechanism in treating diseases relies on the targeting property of the antibody to deliver the toxin molecule into a cell, thereby killing the cell. Drug loading plays a decisive role in drug efficacy. The drug loading of ADC stock solution was determined by mass spectrometry and reversed-phase high-performance liquid chromatography.

### Method 1: Reversed-Phase High-Performance Liquid Chromatography:

(1) Experimental method: The test sample was diluted with ultrapure water to 0.5 mg/mL, reduced with dithiothreitol having a final concentration of 25 mM at 37° C. for 30 min, and then directly subjected to injection, with an injection volume of 5 μL. The chromatographic column was a reversed-phase column (Agilent PLRP-S 1000A (5 μm) 50×2.1 mm), and gradient elution was performed with mobile phases A and B (A: 0.1% formic acid-0.025% trifluoroacetic acid-water, B: 0.1% formic acid-0.025% trifluoroacetic acid-acetonitrile). The flow rate was 0.25 mL/min, the detection wavelength was 280 nm, and the column temperature was 70° C. (2) Data analysis: The positions of the light and heavy chains were distinguished by comparing the spectra of the sample with those of the naked antibody, and then the spectra of the test sample were integrated to calculate the DAR value. The calculation Formula was as follows: LC: 0 (number of conjugated drugs), LC+1: 2 (number of conjugated drugs), HC: 0 (number of conjugated drugs), HC+1: 2 (number of conjugated drugs), HC+2: 4 (number of conjugated drugs), HC+3: 6 (number of conjugated drugs). LC peak area sum=LC peak area+LC+1 peak area; HC peak area sum=HC peak area+HC+1 peak area+HC+2 peak area+HC+3 peak area; LC DAR=Σ (number of conjugated drugs×peak area percentage)/LC peak area sum; HC DAR=Σ (number of conjugated drugs×peak area percentage)/HC peak area sum; DAR=LC DAR+HC DAR.

Method 2: Mass spectrometry: (1) Experimental method: 10 μL of the test sample with a concentration of 1 mg/mL was taken and transferred into a 1.5 mL centrifuge tube, added with 0.5 μL of Rapid PNGase F (Adamas), vortexed and mixed well, and incubated at 37° C. for 60 min. After the N-glycan was cleaved, 6 μL of ultrapure water and 4 μL of 0.5 M dithiothreitol aqueous solution were added to the centrifuge tube, vortexed and mixed well, and incubated at 37° C. for 30 min. After the reaction was completed, centrifugation was performed and the supernatant was taken and transferred into an injection bottle, and injection volume was 2 μL. The column was a reverse phase chromatography column (Agilent PLRP-S 1000A (5 μm) 50×2.1 mm), the gradient elution was performed using mobile phases A and B (A: 0.1% formic acid-water, B: 0.1% formic acid-acetonitrile), the flow rate was 0.5 mL/min, the column temperature was 60° C., the detection wavelength was 280 nm, ESI-Tof (LC-MS) was used to acquire m/z data, the mass spectrometry acquisition mode was positive ion mode, the range for acquisition of m/z was 200-3200, and then the original mass spectrum was deconvoluted by software. (2) Data analysis: The DAR value was calculated using the peak height value by a method similar to the above-mentioned reverse phase high performance liquid chromatography method.

### III. Test Examples

### Biological Evaluation

### Test Example 1: Determination of In Vitro Proliferation Inhibitory Activity of the Compound of Formula (GH) on SK-BR-3 Tumor Cells

### 1. Test Objective:

The objective of this experiment is to detect the in vitro killing activity of the compound of Formula (GH) of the present disclosure on SK-BR-3 tumor cells (human breast cancer cells, Wuhan Procell, Catalog No. CL-0211). The cells were treated with different concentrations of the compound in vitro. After 3 days of culture, the number of viable cells was detected using CCK8 (Cell Counting Kit-8, Catalog No. TS547) reagents, and the in vitro activity of the test compound was evaluated according to the IC₅₀ value.

### 2. Experimental Method:

Taking the test method of in vitro killing activity on SK-BR-3 cells as an example, the following test was performed. It should be understood that this method was also applicable to, but not limited to, in vitro proliferation inhibitory activity tests on other tumor cells (HCC1569, JIMT-1, DIFI, etc.).
(1) Cell culture: SK-BR-3 cells were cultured in RPMI 1640 medium containing 10% FBS (Shanghai BasalMedia Technologies Co., Ltd., Catalog No. G211018).
(2) Cell preparation: SK-BR-3 cells in the logarithmic growth phase were taken, washed once with PBS (phosphate buffered saline, Shanghai BasalMedia Technologies Co., Ltd., Catalog No. E211004), added with 2 to 3 mL of trypsin (0.25% Trypsin-EDTA (1×), Shanghai BasalMedia Technologies Co., Ltd., Catalog No. A121002) to allow digestion for 2 to 3 minutes. After the cells were completely digested, 10 mL of cell culture medium containing 2% FBS was added to wash out the digested cells, centrifugation was performed at 300 g for 5 min, the supernatant was discarded, and then 10 to 20 mL of cell culture medium containing 2% FBS was added to resuspend the cells to prepare a single cell suspension.
(3) Cell plating: The SK-BR-3 single cell suspension was mixed well, the viable cell density was adjusted to 6×10⁴ cells/mL with the cell culture medium containing 2% FBS, the cell suspension after density adjustment was mixed well, and added to a 96-well cell culture plate at 100 μL/well. Only 200 μL of PBS was added to the peripheral wells of the 96-well plate. The culture plate was incubated in an incubator for 24 hours (37° C., 5% CO₂).
(4) Compound preparation: The compound was dissolved in DMSO (dimethyl sulfoxide, SIGMA) to prepare a 2 mM stock solution.

The starting concentrations of small molecule compounds and Dxd were both 600 nM, and the preparation method was as follows.

Dxd (MedChemExpress, Catalog No. HY-13631D, CAS: 1599440-33-1) and 2 mM small molecule compound sample stock solution were diluted to 1200 nM with the cell culture medium containing 2% FBS, and then sterilized by filtration with a 0.22 μM filter. Then, 315 μL of different samples to be tested were added to the first column of a 96-well U-bottomed drug distribution plate, and the sample concentration was 1200 nM; 210 μL of the cell culture medium containing 2% FBS was added to each well of the second to tenth columns. 105 μL of the sample was taken from the first column and added to the 210 μL cell culture medium in the second column and mixed well, and 105 μL was taken and added to the third column, and so on to the tenth column.
(5) Sample loading operation: 100 μL of the sample to be tested of different concentrations was added to the culture plate, and two replicate wells were set for each sample. The culture plate was incubated in an incubator for 3 days (37° C., 5% CO₂).
(6) Color development operation: The 96-well cell culture plate was taken out, the cell culture supernatant was removed by tapping, 100 μL of the RPMI 1640 basal medium (without FBS) containing 10% CCK8 was added, and then it was incubated in an incubator for about 1.5 hours (37° C., 5% CO₂).
(7) Plate reading operation: The 96-well cell culture plate was taken out, placed in an ELISA reader (MD SpectraMax i3X), and A₄₅₀ was determined by the ELISA reader.
(8) Data analysis: Microsoft Excel and Graphpad Prism 5 were used to process and analyze the data.

The results of the above tests were summarized in Table 1:

TABLE 1
IC₅₀ values of the compounds to be tested for inhibiting
proliferation of SK-BR-3 cells in vitro.

| | IC₅₀ |
|---|---|
| Compound No. | (nM) |
| Single-configuration Compound 1-B of Compound 1 in Example | 1.94 |
| 2-1 | |
| Single-configuration Compound 1-A of Compound 1 in Example | 0.93 |
| 2-1 | |
| Single-configuration Compound 2-A of Compound 2 in Example | 5.88 |
| 2-2 | |
| Single-configuration Compound 2-B of Compound 2 in Example | 12.00 |
| 2-2 | |
| Single-configuration Compound 3-A of Compound 3 in Example | 5.91 |
| 2-3 | |
| Single-configuration Compound 3-B of Compound 3 in Example | 2.36 |
| 2-3 | |
| Single-configuration Compound 4-A of Compound 4 in Example | 6.42 |
| 2-4 | |
| Single-configuration Compound 4-B of Compound 4 in Example | 5.01 |
| 2-4 | |
| Single-configuration Compound 5-A of Compound 5 in Example | 9.27 |
| 2-5 | |
| Single-configuration Compound 5-B of Compound 5 in Example | 4.92 |
| 2-5 | |
| Compound 24 in Example 2-24 | 5.79 |
| Compound 25 in Example 2-25 | 5.41 |
| Compound 26 in Example 2-26 | 8.39 |
| Compound 27 in Example 2-27 | 13.90 |
| Compound 28 in Example 2-28 | 15.07 |

Conclusion: The small molecule compounds disclosed in the present invention had significant proliferation inhibitory activity on SK-BR-3 cells.

### Test Example 2: Determination of In Vitro Proliferation Inhibitory Activity of the Compound of Formula (GH) on NCI-N87 Tumor Cells

### 1. Test Objective:

The objective of this experiment is to detect the in vitro killing activity of the compound of Formula (GH) disclosed in the present invention on NCI-N87 tumor cells (human gastric cancer cells, Cell Resource Center, Institute of Basic Medicine, Chinese Academy of Medical Sciences, Catalog No. 3111C0001CCC000481). The cells were treated with different concentrations of the compound in vitro, and after 6 days of culture, the number of viable cells was detected using CCK8 (Cell Counting Kit-8, Catalog No. TS547) reagents, and the in vitro activity of the test compound was evaluated according to the IC₅₀ value.

### 2. Experimental Methods:

(1) Cell culture: NCI-N87 cells were cultured in the RPMI 1640 medium containing 2% FBS (Shanghai BasalMedia Technologies Co., Ltd., Catalog No. L210KJ).
(2) Cell preparation: NCI-N87 cells in the logarithmic growth phase were taken, washed once with PBS (phosphate buffered saline, Shanghai BasalMedia Technologies Co., Ltd., Catalog No. E211004), added with 1 mL of trypsin (0.25% Trypsin-EDTA (1×), Shanghai BasalMedia Technologies Co., Ltd., Catalog No. A121002) to allow digestion for 2 to 3 minutes. After the cells were completely digested, 10 mL of the cell culture medium was added to wash off and obtain the digested cells, centrifugation was performed at 300 times gravitational acceleration (g) for 5 min, the supernatant was discarded, then 10 mL of PBS was added to wash the cells once, centrifugation was performed at 300 times gravitational acceleration (g) for 5 minutes, the supernatant was discarded, and 10 mL of the cell culture medium containing 2% FBS was added to resuspend the cells to prepare a single cell suspension.
(3) Cell plating: The NCI-N87 single cell suspension was mixed, the density of live cells was adjusted to 3×10⁴ cells/mL with 2% FBS cell culture medium, the cell suspension after density adjustment was mixed well, and added to a 96-well cell culture plate at 100 μL/well. Only 200 μL of PBS was added to each of the peripheral wells of the 96-well plate. The plate was incubated in an incubator for 24 hours (37° C., 5% CO₂).
(4) Preparation of sample to be tested:

The sample to be tested was diluted to 3200 nM with cell culture medium containing 2% FBS, then 300 μL of the sample was added to the first well of a 96-well U-bottomed plate with a sample concentration of 3200 nM; and 210 μL of cell culture medium containing 2% FBS was added to each well of the second to tenth columns. 70 μL of the sample was taken from the first column and added to the 210 μL cell culture medium in the second column and mixed well, 70 μL was taken and added to the third column, and so on to the ninth column.
(5) Sample loading operation: 100 μL of the sample to be tested with different concentrations was added to the culture plate, two replicate wells were set for each sample. The culture plate was incubated in an incubator for 6 days (37° C., 5% CO₂).
(6) Color development operation: The 96-well cell culture plate was taken out, the supernatant of the culture medium was removed by tapping, 100 μL of 10% CCK8 (prepared with RPMI 1640 basal culture medium, without FBS) solution was added to each well, and incubation was performed in an incubator for 2 hours.
(7) Plate reading operation: The 96-well cell culture plate was taken out, placed in an ELISA reader (SpectraMax i3X), and A₄₅₀ was determined by the ELISA reader.
(8) Data analysis: Microsoft Excel and Graphpad Prism 5 were used to process and analyze the data.

The results of the above tests were summarized in Table 2:

TABLE 2
IC₅₀ values of the compounds to be tested for inhibiting
proliferation of NCI-N87 cells in vitro.

| | IC₅₀ |
|---|---|
| Compound No. | (nM) |
| Single-configuration Compound 1-B of Compound 1 in Example | 5.36 |
| 2-1 | |
| Single-configuration Compound 2-A of Compound 1 in Example | 1.92 |
| 2-2 | |
| Single-configuration Compound 3-A of Compound 3 in Example | 5.28 |
| 2-3 | |
| Single-configuration Compound 4-A of Compound 4 in Example | 2.40 |
| 2-4 | |
| Single-configuration Compound 5-B of Compound 5 in Example | 2.96 |
| 2-5 | |
| Compound 25 in Example 2-25 | 5.46 |
| Dxd | 10.16 |

Conclusion: The small molecule compounds disclosed in the present disclosure had significant proliferation inhibitory activity on NCI-N87 cells, and the activity was stronger than that of Dxd.

### Test Example 3: In Vitro Proliferation Inhibition Test of the Antibody-Drug Conjugates of the Present Disclosure Targeting HER2 Tumor Cells

### 1. Test Objective:

The objective of this experiment is to detect the inhibitory activity of the HER2-targeting antibody-drug conjugates of the present disclosure on the in vitro proliferation of SK-BR-3 cells (human breast cancer cells, Wuhan Procell, Catalog No. CL-0211), NCI-N87 cells (human gastric cancer cells, Cell Resource Center, Institute of Basic Medicine, Chinese Academy of Medical Sciences, Catalog No. 3111C0001CCC000481) and MDA-MB-468 cells (human breast cancer cells, Cell Center, Shanghai Institutes for Biological Sciences, Chinese Academy of Sciences, Catalog No. 3131C0001000700120). The cells were treated with the compounds having different concentrations in vitro, and after 6 days of culture, the proliferation of the cells was detected using the CCK8 (Cell Counting Kit-8, Catalog No. TS547) reagents, and the in vitro activity was evaluated according to the IC₅₀ values.

### 2. Experimental Methods:

Taking the test method of in vitro proliferation activity of SK-BR-3 cells, NCI-N87 cells and MDA-MB-468 cells as an example, the following test was performed. It should be understood that this method is also applicable to, but not limited to, tests of in vitro proliferation inhibition activity of other tumor cells (HCC1569, JIMT-1, DIFI, etc.).
(1) Cell culture: SK-BR-3 cells, NCI-N87 cells and MDA-MB-468 cells were cultured with RPMI 1640 medium containing 2% FBS (Shanghai BasalMedia Technologies Co., Ltd., Catalog No. L210KJ).
(2) Cell preparation: The cells in the logarithmic growth phase were taken, washed once with PBS (phosphate buffered saline, Shanghai BasalMedia Technologies Co., Ltd., Catalog No. E211004), then added with 1 mL of trypsin (0.25% Trypsin-EDTA (1×), Shanghai BasalMedia Technologies Co., Ltd., Catalog No. A121002) to allow digestion for 2 to 3 minutes. After the cells were completely digested, 10 mL of cell culture medium was added to wash off the digested cells, centrifugation was performed at 300 times gravitational acceleration (g) for 5 min, the supernatant was discarded, and then 10 mL of cell culture medium was added to resuspend the cells to prepare a single cell suspension.
(3) Cell plating: The single cell suspension was mixed well, the viable cell density was adjusted to 3×10⁴ cells/mL with cell culture medium, the cell suspension was mixed well after density adjustment, and added to a 96-well cell culture plate at 100 μL/well. Only 200 μL of PBS was added to each of the peripheral wells of the 96-well plate. The culture plate was incubated in an incubator for 24 hours (37° C., 5% CO₂).
(4) Preparation of sample to be tested:

The sample to be tested was diluted to 400 nM with cell culture medium containing 2% FBS, then 300 μL of the sample was added to the first well of a 96-well U-bottomed plate with a sample concentration of 400 nM; 210 μL of cell culture medium containing 2% FBS was added to each of the wells of the second to tenth columns. 70 μL of the sample was taken from the first column and added to the 210 μL cell culture medium in the second column and mixed well, 70 μL was taken and added to the third column, and so on to the ninth column.
(5) Sample loading operation: 100 μL of the test sample of different concentrations was added to the culture plate, two replicate wells were set for each sample. The culture plate was incubation in an incubator for 6 days (37° C., 5% CO₂).
(6) Color development operation: The 96-well cell culture plate was taken out, the supernatant of the culture medium was removed by tapping, 100 μL of 10% CCK8 (prepared with RPMI 1640 basal culture medium, without FBS) solution was added to each well, and incubated at room temperature for 2 hours.
(7) Plate reading operation: The 96-well cell culture plate was taken out, placed in an ELISA reader (SpectraMax i3X), and A₄₅₀ was determined by the ELISA reader.
(8) Data analysis: Microsoft Excel and Graphpad Prism 5 were used to process and analyze the data.

The results of the above tests were summarized in Table 3:

TABLE 3
IC₅₀ values of in vitro proliferation inhibition of HER2-targeted tumor
cells by the antibody-drug conjugates of the present disclosure.

| | IC₅₀ (nM) | | |
|---|---|---|---|
| Compound No. | SK-BR-3 | NCI-N87 | MDA-MB-468 |
| Example 4-13 ADC-13 | 0.06 | 0.13 | >50 |
| Example 4-98 ADC-98 | 0.09 | 0.10 | >50 |
| Example 4-99 ADC-99 | 0.10 | 0.19 | >50 |
| Example 4-100 ADC-100 | 0.12 | 0.23 | >50 |
| Example 4-104 ADC-104 | 0.11 | 0.15 | >50 |
| Example 4-105 ADC-105 | 0.11 | 0.14 | >50 |
| Example 4-106 ADC-106 | 0.13 | 0.16 | >50 |
| Example 4-107 ADC-107 | 0.09 | 0.16 | >50 |
| Example 4-108 ADC-108 | 0.11 | 0.16 | >50 |
| Example 4-109 ADC-109 | 0.08 | 0.15 | >50 |
| Example 4-110 ADC-110 | 0.09 | 0.15 | >50 |
| Example 4-111 ADC-111 | 0.08 | 0.17 | >50 |
| Example 4-112 ADC-112 | 0.10 | 0.23 | >50 |
| Example 4-113 ADC-113 | 0.08 | 0.20 | >50 |
| Example 4-114 ADC-114 | 0.08 | 0.15 | >50 |
| Example 4-115 ADC-115 | 0.11 | 0.19 | >50 |
| Example 4-116 ADC-116 | 0.08 | 0.16 | >50 |
| Example 4-117 ADC-117 | 0.08 | 0.18 | >50 |
| Example 4-120 ADC-118 | 0.11 | N.D. | >50 |
| Example 4-121 ADC-119 | 0.34 | 0.34 | >50 |
| Example 4-122 ADC-120 | 0.07 | N.D. | >50 |
| Example 4-123 ADC-121 | 0.08 | 0.31 | >50 |
| Example 4-124 ADC-122 | 0.12 | 0.16 | >50 |
| Example 4-125 ADC-123 | 0.08 | N.D. | >50 |
| Example 4-126 ADC-124 | 0.12 | 0.19 | >50 |

N.D.: represents not detected.

Conclusion: The HER2-targeting antibody-drug conjugates of the present disclosure had significant proliferation inhibitory activity on HER2-positive SK-BR-3 and NCI-N87 cells, meanwhile, they had weak proliferation inhibitory activity on HER2-negative MDA-MB-468 cells, indicating good selectivity.

### Test Example 4: Test of Bystander Killing Effect of the Antibody-Drug Conjugates of the Present Disclosure

HER2-positive SK-BR-3 cells (human breast cancer cells, Wuhan Procell, Catalog No. CL-0211) and HER2-negative MDA-MB-468 cells (human breast cancer cells, Cell Center of Shanghai Institutes for Biological Sciences, Chinese Academy of Sciences, Catalog No. 3131C0001000700120) were adjusted to 1.2×10⁵ cells/ml and 4×10⁴ cells/ml with RPMI-1640+10% FBS, respectively, 1 mL of SK-BR-3 and MDA-MB-468 cells each was taken and added to a 6-well plate, mixed and cultured overnight at 37° C. and 5% CO₂. The ADC sample was prepared to a concentration of 4.5 μg/mL (30 nM), and 1 mL was added to the cells to make the total volume 3 mL and the final concentration 10 nM. A solvent control group was set up, and culturing was performed at 37° C. and 5% CO₂ for 5 days. Trypsin (0.25% Trypsin-EDTA (1×), Shanghai BasalMedia Technologies Co., Ltd., Catalog No.: A121002) was added to the cells in the 6-well plate for digestion, 1 mL of FACS buffer (PBS+4% FBS) was added for resuspension, the cells were taken and stained with trypan blue, and counted. The remaining cells were centrifuged. Trastuzumab was diluted to 10 μg/mL with FACS buffer. The cells were resuspended with 200 μL of antibody solution, incubated on ice for 30 minutes, Centrifugation was performed and the supernatant was discarded, washing was performed once with 1 mL of FACS buffer, 200 μL of AF647-labeled sheep anti-human Fc antibody (Jackson, Catalog No.: 109-606-170) solution was added and incubated on ice for 15 minutes, 5 μg/mL PI (Sigma, Catalog No.: 31845) solution was added and incubated on ice for 5 minutes, centrifugation was performed and the supernatant was discarded, washing was performed once with 1 mL FACS buffer, the cells were resuspended with 400 μL of PBS, and analyzed by BD Celesta flow cytometer. The ratio and number of cells of the two types were determined according to the flow cytometry and counting results.

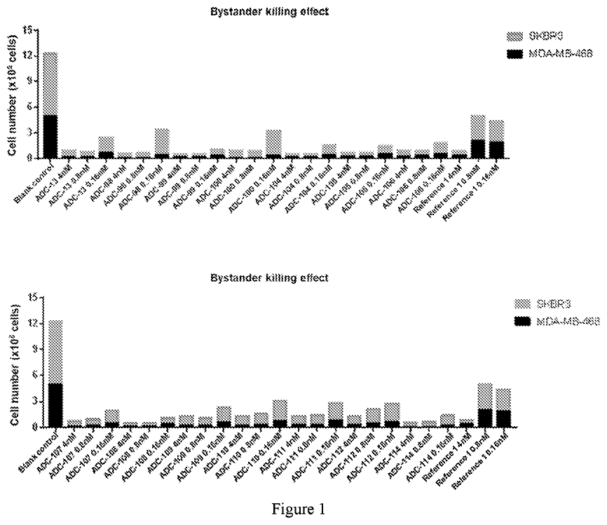

The results of the above tests were summarized in FIG. 1, which showed that the antibody-drug conjugates of the present disclosure had excellent bystander killing effects.

### Test Example 5: Evaluation of Efficacy of Antibody-Drug Conjugates in NCI-N87 Transplanted Tumor Mice

### Test 5A:

### 1. Test Objective:

The objective of this experiment is to detect the efficacy of HER2 antibody ADC drugs on nude mice with human gastric cancer cell NCI-N87 transplanted tumors.

### 2. Test Drugs and Materials:

(1) Test drug: antibody-drug conjugate: 2 mg/kg.
(2) Experimental animals: Balb/c-nude female mice, 6 to 8 weeks old, weighing 20 to 22 g (Gempharmatech), raised in a sterile animal room at 25° C. with a 12-hour day and night cycle, and were allowed to access food and water freely.

### 3. Experimental Methods:

### (1) NCI-N87 Subcutaneous Transplanted Tumor Model in Mice

Inoculation method: NCI-N87 cells in the logarithmic growth period (Cell Resource Center, Institute of Basic Medicine, Chinese Academy of Medical Sciences, 3111C00001CCC000481) were taken, centrifuged at 300 times gravitational acceleration (g) for 7 minutes, washed twice with pre-cooled PBS and counted. The cell density was adjusted to 6e⁷/mL, a total of 10 mL, mixed thoroughly in PBS, dispense into sterile 1.5 mL EP tubes, and placed on ice for later use. 1 mL sterile syringe was used to subcutaneously inoculate the tumor at a volume of 100 μL/mouse, liquid leakage should be avoided.

### (2) Drug Administration and Tumor Volume Detection

When the volume of the transplanted tumor reached 100 to 300 mm³ (on the 8th day after tumor inoculation), the tumor-bearing mice with similar tumor volumes were selected for grouping and administration, 5 mice in each group. Drug administration was performed via tail vein, once on day 0. The tumor volume was measured by a vernier caliper every 2 to 3 days; the length (a) and width (b) of tumor were taken, and volume=a×b×b/2. A tumor growth curve was plotted (in which the tumor volume did not exceed 3000 mm³, and when it reached 3000 mm³, the mice were sacrificed), and the body weight changes in mice were detected at the same time.

### (3) Data Analysis:

All experimental results were expressed as mean±standard error. Graphpad Prism 7.0 software was used to perform statistical analysis on the tumor volume of each group. For tumor volume, one-way ANOVA and Dunnett's test were used for statistical analysis to evaluate the differences between groups for the vehicle control group and all treatment groups. P<0.05 was considered to be significant different.

*P<0.05, **P<0.01, ***P<0.001, ****P<0.0001.

T/C (%)=(T−T₀)/(C−C₀)×100, wherein T and C were the tumor volumes of the experimental group and the control group at the end of the experiment, respectively; T₀ and C₀ were the tumor volumes at the beginning of the experiment.

$$Tumor growth inhibition rate ( T G I ) ( \% ) = 100 - T / C ( \% ) .$$

When the tumor regressed, the tumor growth inhibition rate (TGI)(%)=100−(T−T₀)/T₀×100. If the tumor was smaller than the starting volume, that was, T<T₀ or C<C₀, it was defined as partial tumor regression (PR); if the tumor disappeared completely, it was defined as complete tumor regression (CR).

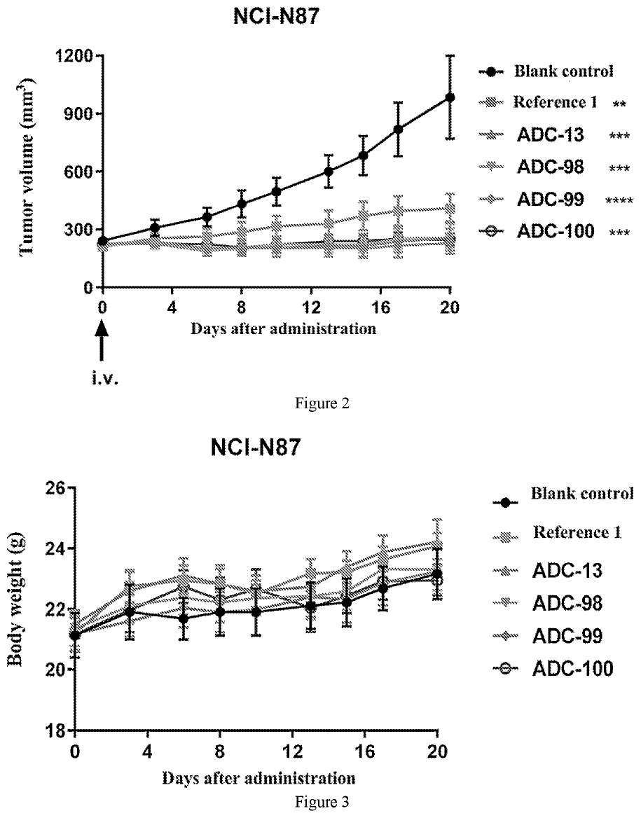

The results of the average tumor volume change, relative tumor growth rate and tumor growth inhibition rate of the above tests were summarized in FIG. 2 and Table 4, which indicated that the antibody-drug conjugates to be tested could effectively inhibit the growth of NCI-N87 transplanted tumors in nude mice, among which ADC-13, ADC-98, ADC-99, and ADC-100 had better tumor inhibition effects on the same batch of tumor cells at the same dose than Reference 1.

TABLE 4

| | Average tumor volume | | Relative tumor | Tumor growth |
|---|---|---|---|---|
| | (mm³) | | proliferation | inhibition |
| Group | Day 0 | Day 20 | rate, T/C % | rate, TGI % |
| Vehicle | 240.50 ± | 984.70 ± | — | — |
| | 20.91 | 215.50 | | |
| Reference 1 | 209.40 ± | 407.20 ± | 26.58 | 73.42 |
| | 19.42 | 77.75 | | |
| ADC-13 | 222.50 ± | 255.80 ± | 4.49 | 95.51 |
| | 15.01 | 83.05 | | |
| ADC-98 | 225.60 ± | 253.80 ± | 3.79 | 96.21 |
| | 18.27 | 48.09 | | |
| ADC-99 | 220.90 ± | 229.10 ± | 1.09 | 98.91 |
| | 14.03 | 39.84 | | |
| ADC-100 | 223.40 ± | 245.30 ± | 2.94 | 97.06 |
| | 15.83 | 31.25 | | |

The body weight changes of experimental mice were summarized in FIG. 3. The body weight of animals of each group was normal during the drug administration process, indicating that the mice had good tolerance to the antibody-drug conjugates to be tested.

### Test 5B:

The same experimental method as the above Test 5A was used, except that the specific batches of NCI-N87 cells in the logarithmic growth phase (Cell Resource Center, Institute of Basic Medicine, Chinese Academy of Medical Sciences, 3111C0001CCC000481) used were different. When the volume of the transplanted tumor reached 100 to 300 mm³ (on the 4th day after tumor inoculation), the tumor-bearing mice with similar tumor volumes were selected for grouping and administration, and the efficacy evaluation of the antibody-drug conjugates in NCI-N87 transplanted tumor mice was performed.

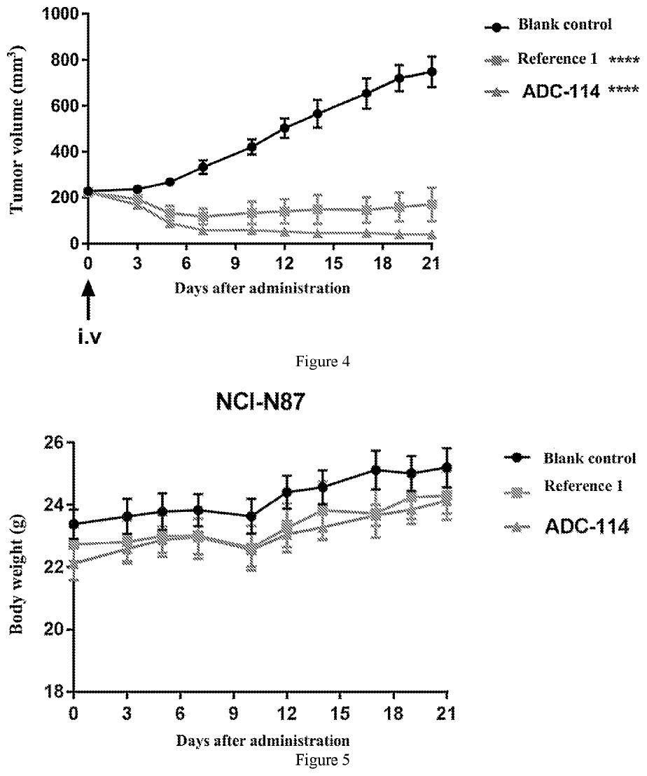

The results of the average tumor volume change, relative tumor growth rate and tumor growth inhibition rate were summarized in FIG. 4 and Table 5, indicating that the tested antibody-drug conjugate ADC-114 could effectively inhibit the growth of NCI-N87 transplanted tumors in nude mice, and its tumor inhibition effect on the same batch of tumor cells at the same dose was better than that of Reference 1.

TABLE 5

| | Average tumor volume | | Relative tumor | Tumor growth |
|---|---|---|---|---|
| | (mm³) | | proliferation | inhibition |
| Group | Day 0 | Day 21 | rate, T/C % | rate, TGI % |
| Vehicle | 228.00 ± | 748.60 ± | — | — |
| | 14.78 | 66.28 | | |
| Reference 1 | 222.90 ± | 169.50 ± | −8.09 | 108.09 |
| | 15.10 | 72.40 | | |
| ADC-114 | 222.60 ± | 39.54 ± | −37.13 | 137.13 |
| | 12.40 | 8.85 | | |

The body weight changes of the experimental mice in the above tests were summarized in FIG. 5. The results showed that the weight of animals in each group was normal during the administration process, indicating that the mice had good tolerance to the antibody-drug conjugates to be tested.

### Test 5C:

The same experimental method as the above Test 5A was used; the difference lied in that the dosage of the antibody-drug conjugates in this test was 6 mg/kg, and the specific batches of the used NCI-N87 cells (Cell Resource Center, Institute of Basic Medicine, Chinese Academy of Medical Sciences, 3111C00001CCC000481) in the logarithmic growth phase were different. When the volume of the transplanted tumor reached 100 to 300 mm³ (on the 4^{th} day after tumor inoculation), the tumor-bearing mice with similar tumor volumes were selected for grouping and administration, and the efficacy evaluation of the antibody-drug conjugates in NCI-N87 transplanted tumor mice was performed.

The results of average tumor volume change, relative tumor growth rate and tumor growth inhibition rate of the test were summarized in FIG. 7 and Table 6, indicating that the tested antibody-drug conjugates could effectively inhibit the growth of NCI-N87 transplanted tumors in nude mice, and at the same dose, ADC-120 and ADC-123 had better tumor inhibition effects on the same batch of tumor cells than Reference 2.

TABLE 6

| | Average tumor volume | | Relative tumor | Tumor growth |
|---|---|---|---|---|
| | (mm³) | | proliferation | inhibition |
| Group | Day 0 | Day 20 | rate, T/C % | rate, TGI % |
| Vehicle | 175.03 ± | 676.06 ± | — | — |
| | 13.53 | 53.36 | | |
| Pertuzumab | 174.15 ± | 687.90 ± | 102.53 | −2.53 |
| | 20.53 | 123.83 | | |
| Reference 2 | 174.80 ± | 137.51 ± | −7.44 | 121.33 |
| | 19.36 | 46.60 | | |
| ADC-120 | 172.19 ± | 96.63 ± | −15.07 | 143.87 |
| | 21.28 | 26.51 | | |
| ADC-123 | 174.26 ± | 49.33 ± | −24.93 | 171.68 |
| | 16.56 | 15.50 | | |

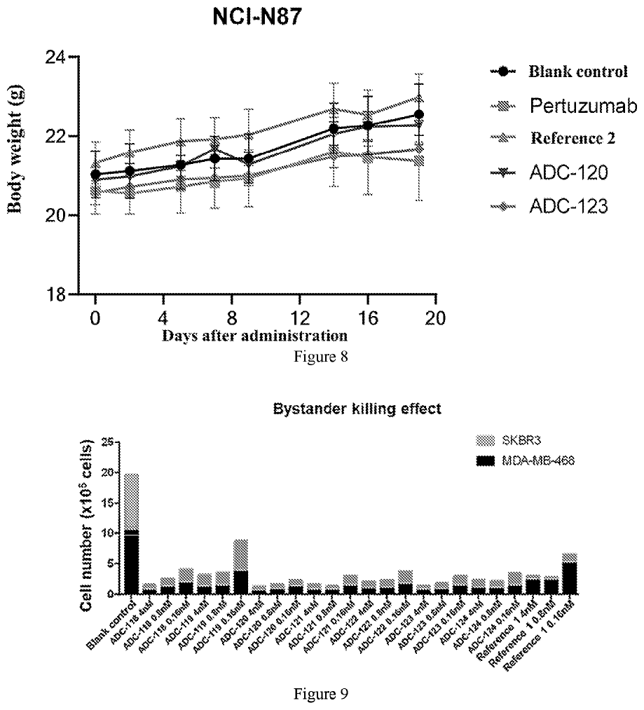

The weight changes of the experimental mice in the above test are summarized in FIG. 8. The results show that the weight of each group of animals was normal during the administration process, indicating that the mice had good tolerance to the tested antibody-drug conjugates.

### Test Example 6: Determination of Plasma Stability of Antibody-Drug Conjugates

### I. Test Objective:

After incubation for a certain period of time, the stability of ADC in the following plasma of four different species was determined: human plasma (collected from volunteers), monkey plasma (Beijing Iphasebio, 032B11.21), mouse plasma (Beijing Iphasebio, 032E13.22), and rat plasma (Beijing Iphasebio, 032D11.11). The ADC samples were diluted to the same concentration with plasma from different species, placed in an incubator at 37° C. for a certain period of time, and then taken out. The free toxin was detected by mass spectrometry (Thermo, TSQ Altis), and the plasma stability of the ADC was evaluated based on the dissociation degree value.

### II. Experimental Method:

1. Sample dilution: The ADC sample was diluted to 200 μg/mL with plasma from different species, with a volume of 2 mL.
2. Filtration sterilization: 2 mL of the ADC sample solution was filtered using 0.22 μm Syringe Driven Filters (Biofil, 220509-051-A) (ensuring that the volume after filtration was >1.8 mL).
3. Sample aliquoting: The filtered ADC sample solution was aliquoted into 1.5 mL EP tubes (Axygen, MCT-150-C-S), 300 μL/tube, labeled and sealed with sealing film.
4. Sample incubation: The aliquoted samples were placed in an incubator at 37° C. for 0, 1, 3, 7, 14, and 21 days, and the sample on Day 0 was directly stored at −80° C.
5. Sample delivery and testing: After incubation, the samples were sent for mass spectrometry, and free toxins were detected by a mass spectrometer.

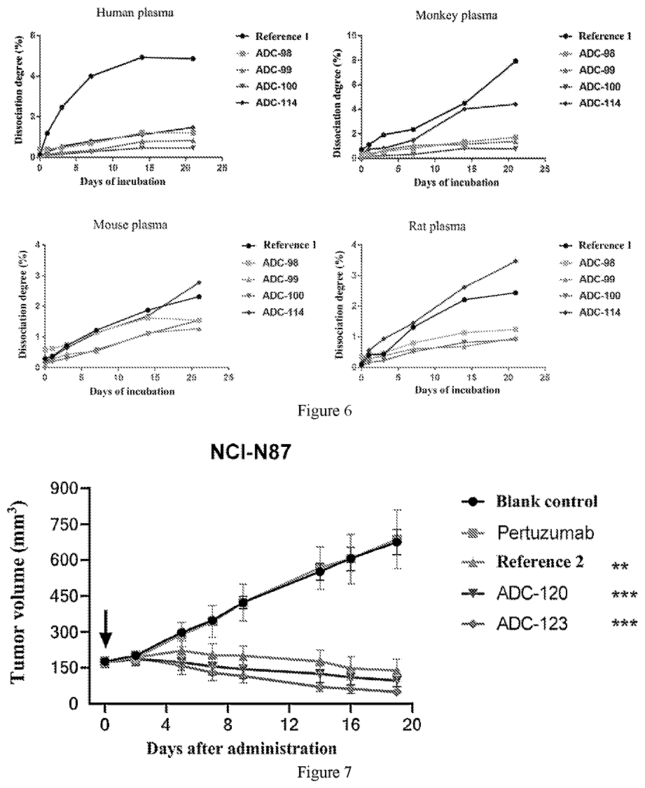

Data analysis: The data were processed and analyzed using Microsoft Excel and Graphpad Prism
5. The results of the above tests were summarized in FIG. 6.

The experimental results showed that the free toxins of the antibody-drug conjugates decreased to a lower level in the plasma of humans, monkeys, mice and rats. On the Day 21, the release rates of free toxins of ADC-98, ADC-99 and ADC-100 did not exceed 2%.

### Test Example 7: Test on Bystander Killing Effect of Pertuzumab Antibody-Drug Conjugate of the Present Disclosure

HER2-positive SK-BR-3 cells (human breast cancer cells, Wuhan Procell, Catalog No. CL-0211) and HER2-negative MDA-MB-468 cells (human breast cancer cells, Cell Center of Shanghai Institutes for Biological Sciences, Chinese Academy of Sciences, Catalog No. 3131C0001000700120) were adjusted to 1.2×10⁵ cells/ml and 4×10⁴ cells/ml with RPMI-1640+10% FBS, respectively, 1 mL of SK-BR-3 and MDA-MB-468 cells each was added to a 6-well plate, mixed and cultured at 37° C. and 5% CO₂ overnight. The ADC sample was prepared to a concentration of 4.5 μg/mL (30 nM), and 1 mL was added to the cells to make the total volume 3 mL and the final concentration 10 nM. A solvent control group was set up and cultured at 37° C. and 5% CO₂ for 5 days. The cells were digested by adding trypsin (0.25% Trypsin-EDTA (1×), Shanghai BasalMedia Technologies Co., Ltd., Catalog No. A121002) to the 6-well plate, and resuspended in 1 mL of FACS buffer (PBS+4% FBS), and the cells were stained with trypan blue and counted. The remaining cells were centrifuged, Pertuzumab was diluted to 10 μg/mL with FACS buffer, the cells were resuspended with 200 μL of antibody solution and incubated on ice for 30 minutes, centrifugation was performed and the supernatant was discarded, washing was performed once with 1 mL of FACS buffer, 200 μL of AF647-labeled sheep anti-human Fc antibody (Jackson, Catalog No.: 109-606-170) solution was added and incubated on ice for 15 minutes, 5 μg/mL PI (Sigma, Catalog No.: 31845) solution was added and incubated on ice for 5 min, centrifugation was performed and the supernatant was discarded, washing was performed once with 1 mL FACS buffer, and the cells were resuspended with 400 μL PBS, and analyzed by BD Celesta flow cytometer. The ratio and number of cells of the kinds were determined according to the flow cytometry and counting results.

The results of the above tests were summarized in FIG. 9, which showed that the Pertuzumab antibody-drug conjugate of the present disclosure had excellent bystander killing effect.

### Test Example 8: Test on In Vitro Proliferation Inhibition of EGFR-Targeted Tumor Cells by the Antibody-Drug Conjugates of the Present Disclosure

### 1. Test Objective:

The objective of this experiment is to detect the inhibitory activity of the EGFR-targeting antibody-drug conjugates of the present disclosure on the in vitro proliferation of MDA-MB-468 cells (human breast cancer cells, Cell Center, Shanghai Institutes for Biological Sciences, Chinese Academy of Sciences, Catalog No. 3131C0001000700120), SK-BR-3 cells (human breast cancer cells, Wuhan Procell, Catalog No. CL-0211), and SW620 cells (human colon cancer cells, Biofeng, bc067). The cells were treated with compounds of different concentrations in vitro, and after 6 days of culture, the proliferation of cells was detected using CCK8 (Cell Counting Kit-8, Catalog No.: TS547) reagents, and the in vitro activity was evaluated according to the IC₅₀ values.

### 2. Experimental Method:

Taking the test method of in vitro proliferation activity of MDA-MB-468 cells, SK-BR-3 cells and SW620 cells as an example, the following test was performed. It should be understood that this method was also applicable to, but not limited to, in vitro proliferation inhibition activity tests on other tumor cells (HCC1569, JIMT-1, DIFI, etc.).
(1) Cell culture: MDA-MB-468 cells and SW620 cells were cultured in L-15 medium containing 10% FBS (Gibco, Catalog No. 11415-064); SK-BR-3 cells were cultured in RPMI 1640 medium containing 10% FBS (Shanghai BasalMedia Technologies Co., Ltd., Catalog No. L210KJ).
(2) Cell preparation: The cells in the logarithmic growth phase were taken, washed once with PBS (phosphate buffered saline, Shanghai BasalMedia Technologies Co., Ltd., Catalog No. E211004), then added with 1 mL of trypsin (0.25% Trypsin-EDTA (1×), Shanghai BasalMedia Technologies Co., Ltd., Catalog No. A121002) for digestion for 2 to 3 minutes. After the cells were completely digested, 10 mL of the corresponding cell culture medium containing 10% FBS was added to wash off the digested cells, centrifugation was performed at 300 times gravitational acceleration (g) for 5 min and the supernatant was discarded, and then 10 mL of cell culture medium was added to resuspend the cells to prepare a single cell suspension.
(3) Cell plating: The single cell suspension was mixed well, the viable cell density was adjusted to 3×10⁴ cells/mL with the corresponding cell culture medium containing 2% FBS, the cell suspension after density adjustment was mixed well, and add to a 96-well cell culture plate at 100 μL/well. Only 200 μL of PBS was added to the peripheral wells of the 96-well plate. The culture plates of MDA-MB-468 cells and SW620 cells were cultured in a CO₂-free incubator for 24 hours (37° C.), and the culture plate of SK-BR-3 cells was cultured in a 5% CO₂ incubator for 24 hours (37° C.).
(4) Preparation of samples to be tested:

The samples to be tested were diluted to 400 nM with the cell culture medium containing 2% FBS corresponding to the cells to be added, and then 300 μL of the sample was added to the first well of a 96-well U-bottomed plate with a sample concentration of 400 nM; 210 μL of the corresponding FBS-containing cell culture medium was added to each well of the second to tenth columns. 70 μL of the sample in the first column was taken and added to the 210 μL cell culture medium in the second column and mix welled, 70 μL was taken and added to the third column, and so on to the ninth column.
(5) Sample loading operation: 100 μL of the sample to be tested with different concentrations was added to the culture plate, and two replicate wells were set for each sample. The culture plates of MDA-MB-468 cells and SW620 cells were placed in a CO₂-free incubator for 6 days (37° C.), and the culture plate of SK-BR-3 cells were placed in a 5% CO₂ incubator for 6 days (37° C.).
(6) Color development operation: The 96-well cell culture plate was taken out, the supernatant of the culture medium was removed by tapping, 100 μL of 10% CCK8 (prepared with RPMI 1640 basal culture medium, without FBS) solution was added to each well, and incubated at room temperature for 2 hours.
(7) Plate reading operation: The 96-well cell culture plate was taken out, placed in an ELISA reader (SpectraMax i3X), and A₄₅₀ was determined by the ELISA reader.
(8) Data analysis: Microsoft Excel and Graphpad Prism 5 were used to process and analyze the data.

The results of the above tests were summarized in Table 7:

TABLE 7
IC₅₀ values of in vitro proliferation inhibition of EGFR-targeted
tumor cells by disclosed antibody-drug conjugates.

| | IC₅₀ (nM) | | |
|---|---|---|---|
| Compound No. | MDA-MB-468 | SK-BR-3 | SW620 |
| Example 4-127 ADC-125 | 0.19 | 9.53 | 22.67 |
| Example 4-128 ADC-126 | 0.21 | N.D. | >50 |
| Example 4-129 ADC-127 | 0.14 | 34.39 | >50 |
| Example 4-130 ADC-128 | 0.21 | 8.63 | 29.92 |
| Example 4-131 ADC-129 | 0.20 | N.D. | >50 |
| Example 4-132 ADC-130 | 0.19 | 44.05 | >50 |
| Example 4-133 ADC-131 | 0.23 | N.D. | >50 |

N.D. represents not detected.

Conclusion: The EGFR-targeting antibody-drug conjugates of the present disclosure had significant proliferation inhibition activity on EGFR-positive MDA-MB-468 cells, meanwhile, they had weak proliferation inhibition activity on EGFR-negative SW620 cells, indicating good selectivity.

### Test Example 9: Test of Bystander Killing Effect of the Antibody-Drug Conjugates of the Present Disclosure on EGFR-Targeted Tumor Cells

EGFR-positive MDA-MB-468 cells (human breast cancer cells, Cell Center, Shanghai Institutes for Biological Sciences, Chinese Academy of Sciences, Catalog No. 3131C0001000700120) and EGFR-negative SW620 cells (human colon cancer cells, Biofeng, bc067) were adjusted to cell density of 1.2×10⁵ cells/ml and 4×10⁴ cells/ml with RPMI-1640+10% FBS, respectively, and 1 mL of MDA-MB-468 and SW620 cells each was added to 6-well plates, mixed and cultured overnight at 37° C. and 5% CO₂. The ADC sample was prepared to a concentration of 4.5 μg/mL (30 nM), and 1 mL was taken and added to the cells to make the total volume 3 mL and the final concentration 10 nM. A solvent control group was set up and cultured at 37° C. and 5% CO₂ for 5 days. The cells were digested by adding trypsin (0.25% Trypsin-EDTA (1×), Shanghai BasalMedia Technologies Co., Ltd., Catalog No.: A121002) to the 6-well plate, and 1 mL ofFACS buffer (PBS+4% FBS) was added to resuspend, and the cells were stained with trypan blue and counted. The remaining cells were centrifuged, Nimotuzumab was diluted to 10 μg/mL with FACS buffer, the cells were resuspended with 200 μL of antibody solution and incubated on ice for 30 min, centrifugation was performed and the supernatant was discarded, washing was performed once with 1 mL FACS buffer, 200 μL of AF647 labeled sheep anti-human Fc antibody (Jackson, Catalog No.: 109-606-170) solution was added and incubated on ice for 15 minutes, 5 μg/mL PI (Sigma, Catalog No.: 31845) solution was added and incubated on ice for 5 minutes, centrifugation was performed and the supernatant was discarded, washing was performed once with 1 mL FACS buffer, and the cells were resuspended with 400 μL PBS and analyzed by BD Celesta flow cytometer. The ratio and number of cells of the two types were determined according to the flow cytometry and counting results.

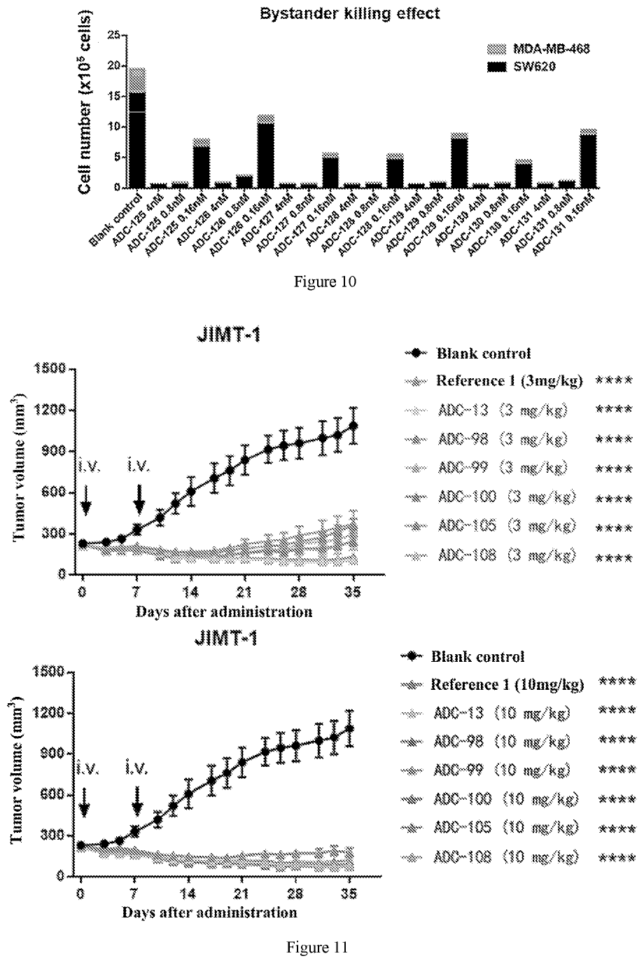

The results of the above tests were summarized in FIG. 10, which showed that the Nimotuzumab antibody-drug conjugate of the present disclosure had excellent bystander killing effect.

### Test Example 10: Evaluation of Efficacy of Antibody-Drug Conjugates in JIMT-1 Transplanted Tumor Mice

### 1. Test Objective:

The objective of this experiment is to detect the efficacy of HER2 antibody ADC drugs on human breast cancer JIMT-1 transplanted tumors in nude mice.

### 2. Test Drugs and Materials:

(1) Test drug: antibody-drug conjugate: 3 mg/kg, 10 mg/kg.
(2) Experimental animals: Balb/c-nude female mice, 6 to 8 weeks old, weighing 20 to 22 g (Gempharmatech), raised in a sterile animal room at 25° C. with a 12-hour day and night cycle, and were allowed to access food and water freely.

### 3. Experimental Methods:

### (1) JIMT-1 Mouse Subcutaneous Transplanted Tumor Model

Inoculation method: JIMT-1 cells (DSMZ, ACC 589v) in the logarithmic growth period were taken, and centrifuged at 300 times gravitational acceleration (g) for 7 minutes. After washing twice with pre-cooled PBS, the cells were counted, adjusted to reach a cell density to 1e8/mL, mixed well with Matrigel (1:1), and aliquoted into sterile 2 mL EP tubes, and placed on ice for later use. 1 mL sterile syringe was used to subcutaneously inoculate the tumor at a volume of 100 μL/mouse, liquid leakage should be avoided.

### (2) Drug Administration and Tumor Volume Detection

When the volume of the transplanted tumor reached 100 to 300 mm³ (around the 4th day after tumor inoculation), tumor-bearing mice with similar tumor volumes were selected for grouping and administration, with 6 mice in each group. Drug administration was performed via tail vein, twice on day 0 and day 7. The tumor volume was measured by a vernier caliper every 2 to 3 days; the length (a) and width (b) of tumor were taken, and volume=a×b×b/2. A tumor growth curve was drown (in which the tumor volume did not exceed 3000 mm³, and when it reached 3000 mm³, the mice were sacrificed), and the changes in mouse body weight was detected at the same time.

### (3) Data Analysis:

All experimental results were expressed as mean±standard error, and the tumor volume of each group was statistically analyzed using Graphpad Prism 7.0 software. For tumor volume, the solvent control group and all treatment groups were statistically analyzed using one-way ANOVA and Dunnett's test to evaluate the differences between groups. P<0.05 was considered to be significantly different.

*P<0.05, **P<0.01, ***P<0.001, ****P<0.0001.

T/C (%)=(T−T₀)/(C−C₀)×100, wherein T and C were the tumor volumes of the experimental group and the control group at the end of the experiment; T₀ and C₀ were the tumor volumes at the beginning of the experiment.

$$Tumor growth inhibition rate ( T G I ) ( \% ) = 100 - T / C ( \% ) .$$

When the tumor regressed, the tumor growth inhibition rate (TGI) (%)=100−(T−T₀)/T₀×100. If the tumor was smaller than the starting volume, that was, T<T₀ or C<C₀, it was defined as partial tumor regression (PR); if the tumor disappeared completely, it was defined as complete tumor regression (CR).

The results of the average tumor volume change, relative tumor growth rate and tumor groth inhibition rate of the above tests were summarized in FIG. 11 and Table 8, which indicated that the antibody-drug conjugates to be tested could effectively inhibit the growth of JIMT-1 transplanted tumors in nude mice, among which ADC-13, ADC-98, ADC-99, ADC-100, ADC-105, and ADC-108 had better tumor inhibition effects on the same batch of tumor cells at the same dose than Reference 1.

TABLE 8

| | Average tumor volume | | Relative tumor | Tumor growth |
|---|---|---|---|---|
| | (mm³) | | proliferation | inhibition |
| Group | Day 0 | Day 35 | rate, T/C % | rate, TGI % |
| Vehicle | 232.33 ± | 1088.05 ± | — | — |
| | 15.2 | 129.9 | | |
| Reference 1 | 215.14 ± | 375.78 ± | 18.77 | 81.23 |
| (3 mg/kg) | 11.4 | 94.07 | | |
| ADC-13 | 218.45 ± | 109.55 ± | −12.73 | 149.85 |
| (3 mg/kg) | 13.64 | 13.38 | | |
| ADC-98 | 217.64 ± | 248.53 ± | 3.61 | 96.39 |
| (3 mg/kg) | 14.06 | 36.91 | | |
| ADC-99 | 217.52 ± | 251.04 ± | 3.92 | 96.08 |
| (3 mg/kg) | 16.07 | 65.63 | | |
| ADC-100 | 217.17 ± | 134.12 ± | −9.71 | 138.24 |
| (3 mg/kg) | 13.18 | 16.65 | | |
| ADC-105 | 216.64 ± | 359.07 ± | 16.64 | 83.36 |
| (3 mg/kg) | 16.43 | 50.97 | | |
| ADC-108 | 217.57 ± | 300.96 ± | 9.75 | 90.25 |
| (3 mg/kg) | 13.27 | 40.77 | | |
| Reference 1 | 229.07 ± | 177.41 ± | −6.59 | 122.55 |
| (10 mg/kg) | 13.6 | 36.03 | | |
| ADC-13 | 213.62 ± | 81.97 ± | −16.79 | 161.63 |
| (10 mg/kg) | 11.89 | 5.1 | | |
| ADC-98 | 214.51 ± | 95.76 ± | −15.14 | 155.36 |
| (10 mg/kg) | 9.37 | 8.8 | | |
| ADC-99 | 219.68 ± | 120.74 ± | −12.62 | 145.04 |
| (10 mg/kg) | 11.8 | 16.79 | | |
| ADC-100 | 216.06 ± | 81.01 ± | −17.22 | 162.51 |
| (10 mg/kg) | 7.6 | 5.05 | | |
| ADC-105 | 219.71 ± | 81.19 ± | −17.66 | 163.04 |
| (10 mg/kg) | 12.04 | 13.82 | | |
| ADC-108 | 220.67 ± | 77.6 ± | −18.24 | 164.84 |
| (10 mg/kg) | 12.14 | 9.67 | | |

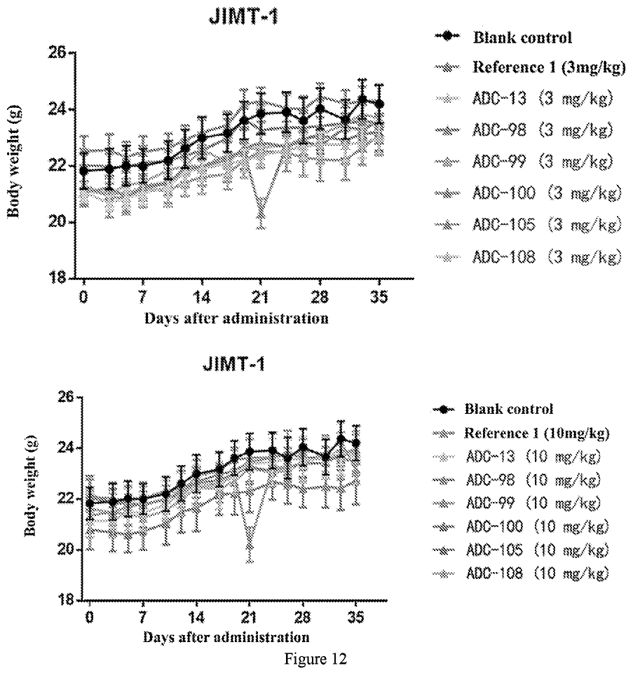

The body weight changes of the experimental mice in the above test were summarized in FIG. 12. The results showed that the body weight of animals of each group was normal during the administration process, indicating that the mice had good tolerance to the antibody-drug conjugates to be tested.

### Test Example 11: Evaluation of Efficacy of Antibody-Drug Conjugates in NCI-H322 Transplanted Tumor Mice

### 1. Test Purpose:

The purpose of this experiment is to detect the efficacy of HER3 antibody ADC drugs on nude mice with human lung cancer transplanted tumors. 2. Test Drugs and Materials:
(1) Test drug: antibody-drug conjugate: 6 mg/kg.
(2) Experimental animals: B-NDG female mice, 6 to 8 weeks old, weighing 20 to 22 g (Biosai Figure), raised in a sterile animal room at 25° C. with a 12-hour day and night cycle, and were allowed to access food and water freely.

### 3. Experimental Methods:

### (1) NCI-H322 Mouse Subcutaneous Transplant Tumor Model:

Inoculation method: NCI-H322 cells (human lung cancer cells, China Food and Drug Inspection Institute, 16 generations) in the logarithmic growth period were taken, centrifuged at 300 times gravitational acceleration (g) for 7 minutes, washed twice with pre-cooled PBS, and counted. The cells were adjusted to reach a cell density of 6e⁷/mL, mixed thoroughly with Matrigel (1:1), aliquoted into sterile 2 mL EP tubes, and placed on ice for later use. 1 mL sterile syringe was used to subcutaneously inoculate the tumor at a volume of 100 μL/cell, wherein liquid leakage should be avoided.

### (2) Administration and Tumor Volume Detection

When the volume of the transplanted tumor reached 100 to 300 mm³ (about the 20^{t} day after tumor inoculation), tumor-bearing mice with similar tumor volumes were selected for grouping and administration, with 6 mice in each group. The drug was administered via tail vein, once on day 0. The tumor volume was measured every 2 to 3 days using a vernier caliper. The length (a) and width (b) of the tumor were taken, and volume=a×b×b/2. A tumor growth curve was drawn (the tumor volume did not exceed 3000 mm³, and when it reached 3000 mm³, the mice were sacrificed). The body weight changes of the mice were also detected.

### (3) Data Analysis:

All experimental results were expressed as mean±standard error, and the tumor volume of each group was statistically analyzed using Graphpad Prism 7.0 software. The control group and all treatment groups were statistically analyzed using one-way ANOVA and Dunnett's test to evaluate the differences between groups. P<0.05 was considered to be significant different.

*P<0.05, **P<0.01, ***P<0.001, ****P<0.0001.

T/C (%)=(T−T₀)/(C−C₀)×100, wherein T and C were the tumor volumes of the experimental group and the control group at the end of the experiment; T₀ and C₀ were the tumor volumes at the beginning of the experiment.

$$Tumor growth inhibition rate ( T G I ) ( \% ) = 100 - T / C ( \% ) .$$

When the tumor regressed, the tumor growth inhibition rate (TGI) (%)=100−(T−T₀)/T₀×100.

When the tumor volume decreased, that was, T<T₀ or C<C₀, it was defined as partial tumor regression (PR); if the tumor disappeared completely, it was defined as complete tumor regression (CR).

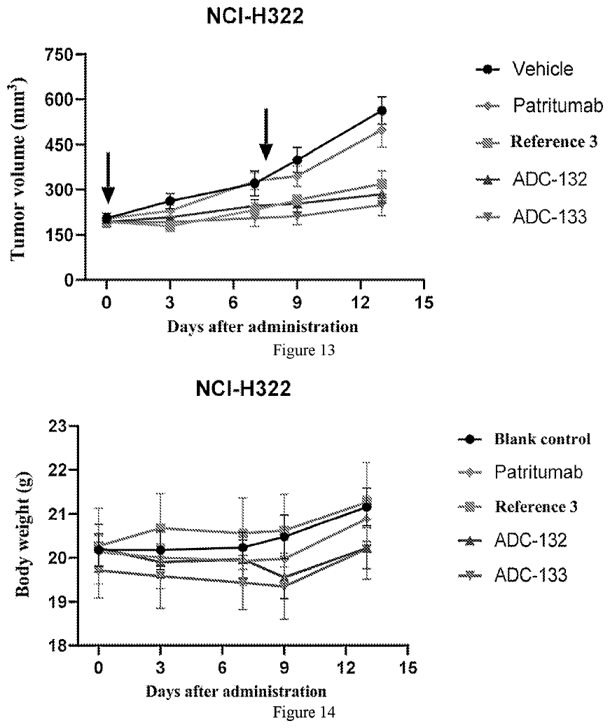

The average tumor volume change, relative tumor growth rate, and tumor growth inhibition rate in the above test were summarized in FIG. 13 and Table 9, which indicated that the antibody-drug conjugates tested could effectively inhibit the growth of NCI-H322 transplanted tumors in nude mice, and at the same dose, the tumor growth inhibition effect of ADC-132 and ADC-133 on the same batch of tumor cells was better than that of Reference 3.

TABLE 9

| | Average tumor | | Relative tumor | Tumor growth |
|---|---|---|---|---|
| | volume (mm³) | | proliferation | inhibition |
| Group | Day 0 | Day 13 | rate, T/C % | rate, TGI % |
| Vehicle | 205.60 ± | 562.96 ± | — | — |
| | 16.32 | 45.13 | | |
| Patritumab | 204.21 ± | 498.73 ± | 82.41 | 17.58 |
| | 17.02 | 57.08 | | |
| Reference 3 | 192.93 ± | 318.18 ± | 35.04 | 64.95 |
| | 13.66 | 43.47 | | |
| ADC-132 | 193.16 ± | 284.38 ± | 25.52 | 74.47 |
| | 17.56 | 29.41 | | |
| ADC-133 | 192.71 ± | 248.97 ± | 15.74 | 84.25 |
| | 16.56 | 35.71 | | |

The body weight changes of the experimental mice in the above test were summarized in FIG. 14. The results showed that the body weight of animals of each group was normal during the administration process, indicating that the mice had good tolerance to the antibody-drug conjugates tested.

### Test Example 12: Pharmacokinetics of Antibody-Drug Conjugates of the Present Disclosure in Rats

### 1. Test Purpose:

The purpose of this experiment is to study the pharmacokinetic characteristics of HER2 antibody ADC drugs and provide a reference for subsequent research.

### 2. Test Drugs and Materials:

(1) Test drug: HER2 antibody-drug conjugate: 3 mg/kg.
(2) Experimental animals: SD rats, 6 to 8 weeks old, weighing 180 to 200 g (Sichuan Weitong Lihua Experimental Animal Technology Co., Ltd.), raised in a sterile animal room at 25° C. with a 12-hour day and night cycle, and were allowed to access food and water freely.

### 3. Experimental Methods:

4 Rats, half female and half male, were divided into 3 groups, namely, ADC-105, ADC-107, and ADC-98, wherein the animals were administered with single dose, at a dosing volume of 10 mL/kg.

Sampling time: Blood samples were collected once before administration, and at 5 min, 7 h, 24 h, 72 h, 168 h, 336 h, and 504 h after administration. Sampling method: Blood samples were collected from jugular vein of rats.

Blood sample processing: After the blood was collected, it was placed in an EDTA anticoagulant tube, and the sample tube was gently inverted several times to ensure well mixing. After the blood was collected, it was placed in a 37° C. constant temperature oven for 30 minutes, centrifuged at 4° C. and 4500 rpm for 12 minutes, then the plasma was pipetted, placed in a −20° C. refrigerator, and transferred to a ultra-low temperature refrigerator (−60° C. and below) after 24 hours. Sample analysis: The established ELISA method was used to analyze the blood concentrations of antibody and ADC drug in serum. The existing LC-MS/MS method was used to analyze the blood concentration of cytotoxic drug in plasma.

The experimental results were shown in Table 10

TABLE 10

| | | T_{1/2} | Tₘₐₓ | Cₘₐₓ | AUC₀₋ₜ |
|---|---|---|---|---|---|
| Analytes | | (h) | (h) | (ug/mL) | (h · ug/mL) |
| Experimental | ADC-105 | 160.20 | 0.083 | 93.9 | 6604.91 |
| group I | ADC-105 total | 183.67 | 0.083 | 69.41 | 6703.19 |
| | antibody | | | | |
| | 2-A | 91.56 | 0.083 | 0.0017 | 0.039 |
| Experimental | ADC-107 | 132.97 | 0.083 | 124.16 | 9309.29 |
| group II | ADC-107 total | 170.48 | 0.083 | 102.15 | 9093.86 |
| | antibody | | | | |
| | 1-B | 5.13 | 0.083 | 0.0034 | 0.014 |
| Experimental | ADC-98 | 141.00 | 0.083 | 110.81 | 8699.15 |
| group III | ADC-98 total | 163.15 | 0.083 | 91.74 | 9172.86 |
| | antibody | | | | |
| | 1-B | N.A. | 0.083 | 0.0003 | 0.0013 |

N.A. represents that the data was too small to fit.

Experimental Conclusion: According to the results in Table 10, no pharmacokinetic differences were observed between the the antibody-drug conjugates (ADCs) of the present disclosure and the total antibody, indicating that the antibody-drug conjugates of the present disclosure had good stability in plasma in vivo. At the same time, low blood concentrations of cytotoxic drugs in plasma were detected, which indicated that due to the stability of the antibody-drug conjugates of the present disclosure and the short systemic half-life of the corresponding cytotoxins, the systemic exposure of each cytotoxic drug was low, and thus the ADCs had a higher safety.

### Test Example 13: Test of Toxicity of the Antibody-Drug Conjugates of the Present Disclosure in Rats

### 1. Test Purpose:

The purpose of this experiment is to study the toxicity characteristics of HER2 antibody ADC drugs and provide a reference for subsequent research.

### 2. Test Drugs and Materials:

(1) Test drug: HER2 antibody-drug conjugate: 20 mg/kg, 60 mg/kg, 197 mg/kg.
(2) Experimental animals: SD rats, 6 to 8 weeks old, weighing 180 to 200 g (Sichuan Weitonglihua Experimental Animal Technology Co., Ltd.), raised in a sterile animal room at 25° C. with a 12-hour day and night cycle, and were allowed to access food and water freely.

### 3. Experimental Method:

Each group had 4 rats, half female and half male. The rats were divided into 3 groups and given ADC-98, ADC-100, and ADC-113 respectively, with a single dose of 10 mL/kg via tail vein injection.

The survival and clinical symptoms of the rats were then observed every other day.

### 4. Experimental Results:

The rats survived for more than 21 days at each dose.

### 5. Experimental Conclusion:

According to the results of this test example, the maximum tolerable dose of the antibody-drug conjugates of the present disclosure in rats was greater than 197 mg/kg, and thus they had high safety.

### Test Example 14: Test of Bystander Killing Effect of the Antibody-Drug Conjugates of the Present Disclosure on HER2-Targeted Tumor Cells

HER2-positive SK-BR-3 cells (human breast cancer cells, Wuhan Procell, Catalog No. CL-0211) and HER2-negative MDA-MB-468 cells (human Breast cancer cells, Cell Center, Shanghai Institutes for Biological Sciences, Chinese Academy of Sciences, Catalog No. 3131C0001000700120) were adjusted with RPMI-1640+10% FBS to reach cell density of 1.2×10⁵ cells/ml and 4×10⁴ cells/ml, respectively. 1 mL of each of SK-BR-3 and MDA-MB-468 cells was added to a 6-well plate and cultured overnight at 37° C. with 5% CO₂. The sample ADC-113 was prepared to a concentration of 4.5 μg/mL (30 nM), and 1 mL was added to the cells to make the total volume 3 mL and the final concentration 10 nM. A solvent control group was set up and cultured at 37° C. and 5% CO₂ for 5 days. The cells were digested by adding trypsin (0.25% Trypsin-EDTA (1×), Shanghai BasalMedia Technologies Co., Ltd., Catalog No.: A121002) to the cells in the 6-well plate, and added with 1 mL of FACS buffer (PBS+4% FBS) to resuspend. The cells were stained with trypan blue and counted. The remaining cells were centrifuged, and Trastuzumab was diluted to 10 μg/mL in FACS buffer. The cells were resuspended with 200 μL of antibody solution and incubated on ice for 30 min. The cells were centrifuged and the supernatant was discarded. The cells were washed once with 1 mL ofFACS buffer, and 200 μL of AF647-labeled goat anti-human Fc antibody (Jackson, Catalog No.: 109-606-170) solution was added and incubated on ice for 15 min, 5 μg/mL PI (Sigma, Catalog No.: 31845) solution was added and incubated on ice for 5 min, centrifugation was performed and the supernatant was discarded, washing was performed once with 1 mL FACS buffer and the cells were resuspended in 400 μL PBS. BD Celesta flow cytometer was used for detection and analysis. The ratio and number of cells were determined based on the flow cytometry and counting results.

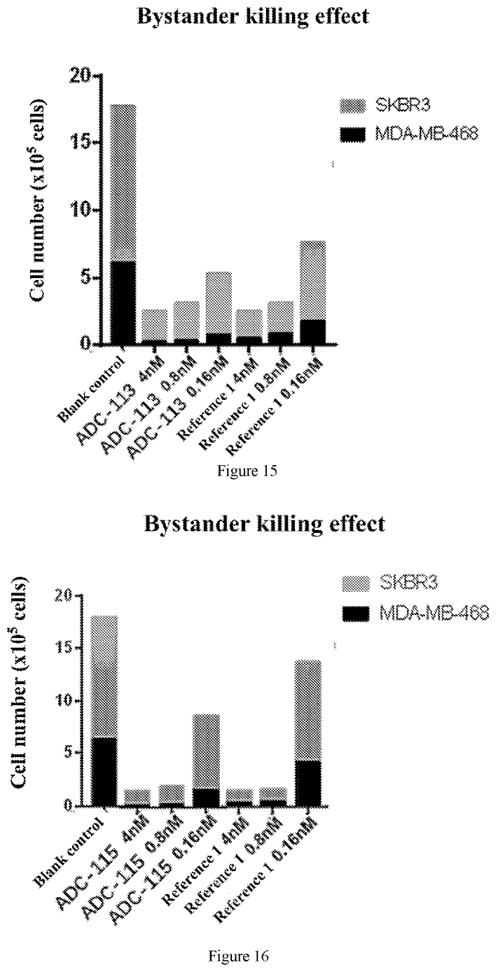

The results of the above tests were summarized in FIG. 15, which indicated that the antibody-drug conjugate of the present disclosure had excellent bystander killing effect.

### Test Example 15: Test of Bystander Killing Effect of the Antibody-Drug Conjugates of the Present Disclosure on HER2-Targeted Tumor Cells

HER2-positive SK-BR-3 cells (human breast cancer cells, Wuhan Procell, Catalog No. CL-0211) and HER2-negative MDA-MB-468 cells (human breast cancer cells, Cell Center, Shanghai Institutes for Biological Sciences, Chinese Academy of Sciences, Catalog No. 3131C0001000700120) were adjusted with RPMI-1640+10% FBS to reach cell density of 1.2×10⁵ cells/ml and 4×10⁴ cells/ml, respectively. 1 mL of each of SK-BR-3 and MDA-MB-468 cells was added to a 6-well plate, mixed and cultured overnight at 37° C. and 5% CO₂. Sample ADC-115 was prepared to a concentration of 4.5 μg/mL (30 nM), and 1 mL was taken and added to the cells to make the total volume 3 mL and the final concentration 10 nM. A solvent control group was set up and cultured at 37° C. and 5% CO₂ for 5 days. Trypsin (0.25% Trypsin-EDTA (1×), Shanghai BasalMedia Technologies Co., Ltd., Catalog No.: A121002) was added to the 6-well plate for digestion. The cells were resuspend in 1 mL FACS buffer (PBS+4% FBS), stained with trypan blue, and counted. The remaining cells were centrifuged, and Trastuzumab was diluted to 10 μg/mL in FACS buffer. The cells were resuspended with 200 μL of antibody solution and incubated on ice for 30 min. The cells were centrifuged and the supernatant was discarded. The cells were washed once with 1 mL of FACS buffer, and 200 μL of AF647-labeled goat anti-human Fc antibody (Jackson, Catalog No.: 109-606-170) solution was added and incubated on ice for 15 min, 5 μg/mL PI (Sigma, Catalog No.: 31845) solution was added and incubated on ice for 5 min, centrifugation was performed and the supernatant was discarded, the cells were washed once with 1 mL FACS buffer, resuspended by adding 400 μL of PBS, and analyzed by BD Celesta flow cytometer. The ratio and number of cells were determined based on the flow cytometry and counting results.

The results of the above tests were summarized in FIG. 16, which indicated that the antibody-drug conjugate of the present disclosure had excellent bystander killing effect.

### Test Example 16: Evaluation of Efficacy of Antibody-Drug Conjugates in NCI-N87 Transplanted Tumor Mice

1. Test Purpose: The Purpose of this Experiment is to Detect the Efficacy of HER2 Antibody ADC Drugs on Nude Mice with Human Gastric Cancer Cell NCI-N87 Transplanted Tumor.

### 2. Test Drugs and Materials:

(1) Test drug: antibody-drug conjugate: 2 mg/kg, 6 mg/kg.
(2) Experimental animals: Balb/c-nude female mice, 6 to 8 weeks old, weighing 20 to 22 g (Gempharmatech), raised in a sterile animal room at 25° C. with 12-hour day and night cycle, and were allowed to access food and water freely.

### 3. Experimental Methods:

### (1) NCI-N87 Subcutaneously Transplanted Tumor Mouse Model

Inoculation method: NCI-N87 cells in the logarithmic growth phase (Cell Resource Center, Institute of Basic Medical Sciences, Chinese Academy of Medical Sciences, 3111C0001CCC000481) were taken, and centrifuged at 300 times gravitational acceleration (g) for 7 min. The cells were washed twice with pre-cooled PBS and counted, adjusted to reach a cell density to 6e⁷/mL, a total of 10 mL, mixed thoroughly in PBS, aliquoted into sterile 1.5 mL EP tubes, and placed on ice for later use. 1 mL sterile syringe was used to subcutaneously inoculate the tumor at a volume of 100 μL/mouse, in which the liquid leakage should be avoided.

### (2) Drug Administration and Tumor Volume Detection

When the volume of transplanted tumor reached 100 to 300 mm³ (on the 8th day after tumor inoculation), tumor-bearing mice with similar tumor volumes were selected for grouping and administration, with 5 mice in each group. Drugs were administered via tail vein. For the 2 mg/kg group, the drug was administered once on day 0 and once on day 7. For the 6 mg/kg group, the drug was administered once on day 0. Tumor volume was measured every 2 to 3 days using a vernier caliper. The length (a) and width (b) of tumor were taken, and volume=a×b×b/2. Tumor growth curves were drawn (the tumor volume did not exceed 3000 mm³, and when it reached 3000 mm³, the mice were sacrificed). The body weight changes of the mice were also detected.

### (3) Data Analysis:

All experimental results were expressed as mean±standard error. Graphpad Prism 7.0 software was used to perform statistical analysis on the tumor volume of each group. For tumor volume, one-way ANOVA and Dunnett's test were used for statistical analysis of the vehicle control group and all treatment groups to evaluate the differences between groups. P<0.05 was considered to be significantly different.

*P<0.05, **P<0.01, ***P<0.001, ****P<0.0001.

T/C (%)=(T−T₀)/(C−C₀)×100, wherein T and C were the tumor volumes of the experimental group and the control group at the end of the experiment; T₀ and C₀ were the tumor volumes at the beginning of the experiment.

$$Tumor growth inhibition rate ( T G I ) ( \% ) = 100 - T / C ( \% ) .$$

When the tumor regressed, the tumor growth inhibition rate (TGI) (%)=100−(T−T₀)/T₀×100. If the tumor was smaller than the starting volume, that was, T<T₀ or C<C₀, it was defined as partial tumor regression (PR); if the tumor disappeared completely, it was defined as complete tumor regression (CR).

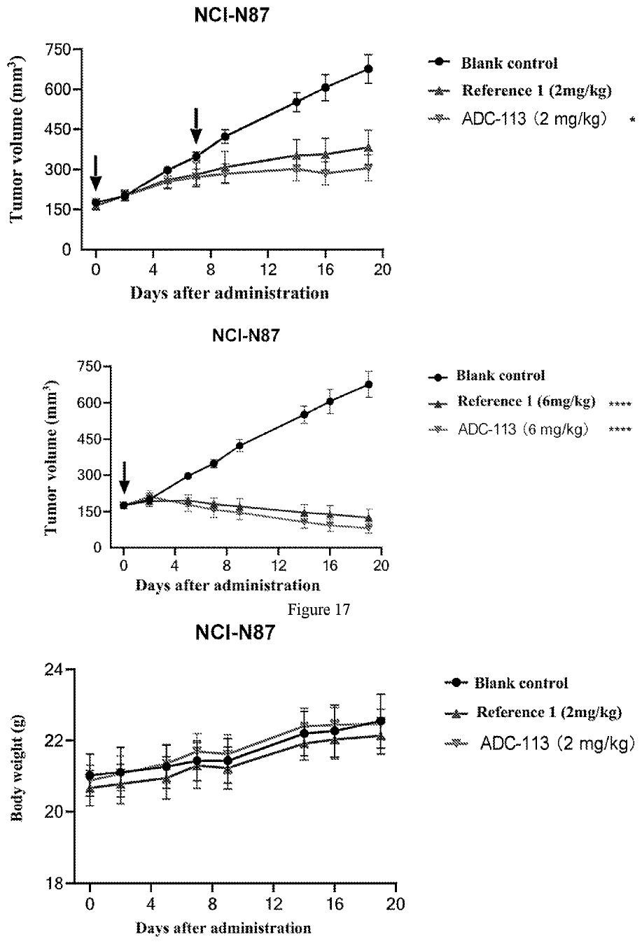

The results of the average tumor volume change, relative tumor growth rate and tumor growth inhibition rate of the above test were summarized in FIG. 17 and Table 11, which indicated that the antibody-drug conjugates tested could effectively inhibit the growth of NCI-N87 transplanted tumors in nude mice, and at the same dose, ADC-113 had a better tumor inhibition effect on the same batch of tumor cells than Reference 1.

TABLE 11

| | Average tumor volume | | Relative tumor | Tumor growth |
|---|---|---|---|---|
| | (mm³) | | proliferation | inhibition |
| Group | Day 0 | Day 19 | rate, T/C % | rate, TGI % |
| Vehicle | 175.03 ± | 676.06 ± | — | — |
| | 13.53 | 53.36 | | |
| Reference 1 | 162.79 ± | 382.35 ± | 43.82 | 56.17 |
| (2 mg/kg) | 15.37 | 64.42 | | |
| ADC-113 | 175.11 ± | 305.31 ± | 25.98 | 74.013 |
| (2 mg/kg) | 13.71 | 49.51 | | |
| Reference 1 | 175.81 ± | 125.09 ± | −10.12 | 128.84 |
| (6 mg/kg) | 14.42 | 35.92 | | |
| ADC-113 | 175.58 ± | 82.22 ± | −18.63 | 153.16 |
| (6 mg/kg) | 13.64 | 20.91 | | |

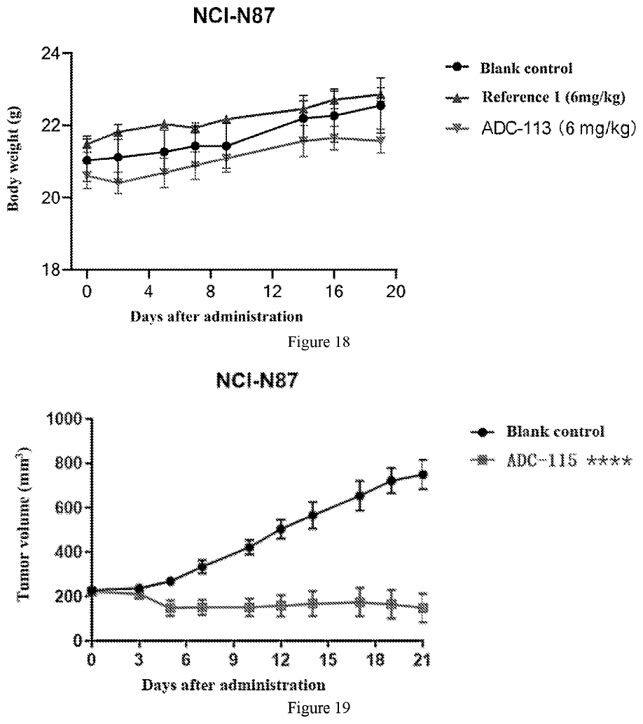

The body weight changes of the experimental mice in the above test were summarized in FIG. 18. The results showed that the body weight of the animals in each group was normal during the administration process, indicating that the mice had good tolerance to the antibody-drug conjugates tested.

### Test Example 17: Evaluation of Efficacy of Antibody-Drug Conjugates in NCI-N87 Transplanted Tumor Mice

### 1. Test Objective:

The objective of this experiment is to detect the efficacy of HER2 antibody ADC drugs on nude mice with human gastric cancer cell NCI-N87 transplanted tumor.

### 2. Test Drugs and Materials:

(1) Test drug: antibody-drug conjugate: 2 mg/kg.
(2) Experimental animals: Balb/c-nude female mice, 6 to 8 weeks old, weighing 20 to 22 g (Gempharmatech), raised in a sterile animal room at 25° C. with 12 h day and night cycle, and were allowed to access food and water freely.

### 3. Experimental Methods:

### (1) NCI-N87 Subcutaneously Transplanted Tumor Mouse Model

Inoculation method: NCI-N87 cells in the logarithmic growth phase (Cell Resource Center, Institute of Basic Medical Sciences, Chinese Academy of Medical Sciences, 3111C0001CCC000481) were taken, and centrifuged at 300 times gravitational acceleration (g) for 7 min. The cells were washed twice with pre-cooled PBS and counted, adjusted to reach a cell density of 6e7/mL, a total of 10 mL, mixed thoroughly in PBS, aliquoted into sterile 1.5 mL EP tubes, and placed on ice for later use. 1 mL sterile syringe was used to subcutaneously inoculate the tumor at a volume of 100 μL/mouse, liquid leakage should be avoided.

### (2) Drug Administration and Tumor Volume Detection

When the volume of the transplanted tumor reached 100 to 300 mm³ (on the 8th day after tumor inoculation), tumor-bearing mice with similar tumor volumes were selected for grouping and administration, with 5 mice in each group. Drugs were administered via tail vein, once on day 0. Tumor volume was measured every 2 to 3 days using a vernier caliper. The length (a) and width (b) of the tumor were taken, and volume=a×b×b/2. Tumor growth curves were drawn (the tumor volume did not exceed 3000 mm³, and when it reached 3000 mm³, the mice were sacrificed), and the body weight changes of mice were detected at the same time.

### (3) Data Analysis:

All experimental results were expressed as mean±standard error. Graphpad Prism 7.0 software was used to perform statistical analysis on the tumor volume of each group. For tumor volume, one-way ANOVA and Dunnett's test were used for statistical analysis of the vehicle control group and all treatment groups to evaluate the differences between groups. P<0.05 was considered to be significantly different.

*P<0.05, **P<0.01, ***P<0.001, ****P<0.0001.

T/C (%)=(T-T0)/(C−C0)×100, where T and C are the tumor volumes of the experimental group and the control group at the end of the experiment; T0 and C0 are the tumor volumes at the beginning of the experiment.

$$Tumor inhibition rate ( T G I ) ( \% ) = 100 - T / C ( \% ) .$$

When the tumor regressed, the tumor growth inhibition rate (TGI) (%)=100−(T−T₀)/T₀×100. If the tumor was smaller than the starting volume, that was, T<T₀ or C<C₀, it was defined as partial tumor regression (PR); if the tumor disappeared completely, it was defined as complete tumor regression (CR).

The results of the average tumor volume change, relative tumor growth rate and tumor growth inhibition rate of the above test were summarized in FIG. 19 and Table 12, which indicated that the antibody-drug conjugates tested could effectively inhibit the growth of NCI-N87 transplanted tumors in nude mice.

TABLE 12

| | Average tumor volume | | Relative tumor | Tumor growth |
|---|---|---|---|---|
| | (mm³) | | proliferation | inhibition |
| Group | Day 0 | Day 21 | rate, T/C % | rate, TGI % |
| Vehicle | 227.97 ± | 748.59 ± | — | — |
| | 14.78 | 66.28 | | |
| ADC-115 | 222.68 ± | 147.67 ± | −14.41 | 133.69 |
| | 12.18 | 64.89 | | |

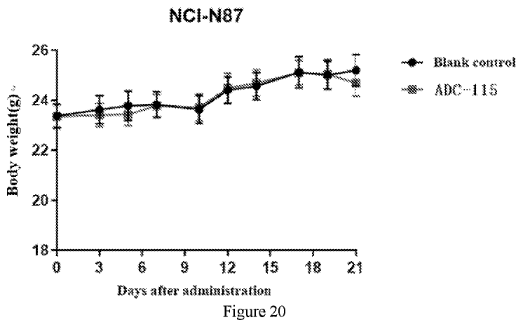

The body weight changes of the experimental mice in the above test were summarized in FIG. 20. The results showed that the body weight of animals of each group was normal during the administration process, indicating that the mice had good tolerance to the antibody-drug conjugate tested.

### Test Example 18: Determination of Plasma Stability of Antibody-Drug Conjugate

The plasma stability of ADC-115 was evaluated by the same method as in Test Example 6.

The experiment showed that the free toxin of antibody-drug conjugate ADC-115 is released less in the plasma of humans, monkeys, mice and rats, and the release rate of its free toxin did not exceed 2% on the 21^{st} day.

The above is the exemplary descriptions of the embodiments of the technical solution of the present disclosure. It should be understood that the protection scope of the present disclosure is not limited to the above embodiments. Any modifications, equivalent substitutions, improvements, etc. made by a person skilled in the art within the spirit and principle of the present disclosure shall be included in the protection scope of the claims of the present application.

1. A conjugate represented by the following Formula (C), or a stereoisomer, racemate, tautomer, isotopologue, isotope-labeled compound, nitroxide or pharmaceutically acceptable salt thereof: 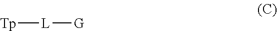
wherein, Tp represents a targeting moiety;
L is selected from a chemical bond or a linker;
G represents a group represented by the following Formula (G): 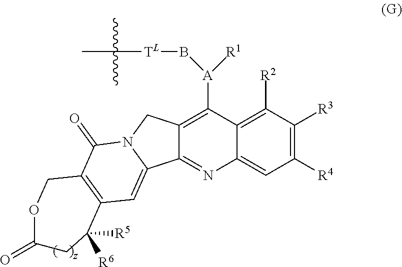
wherein, A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a ring structure which is unsubstituted or substituted with one, two or more R^{E}, for example, the ring structure is a 5- to 10-membered ring structure, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a ring structure which is unsubstituted or substituted with one, two or more R^{F}, for example, the ring structure is a 4- to 10-membered ring structure, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of alkyl, alkyloxy, halogen, hydroxyl, sulfhydryl, amino, cyano;
R⁵ is hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy, alkyl, alkyloxy, aryl, arylalkyl, aryloxy, heteroaryl, heteroarylalkyl, heteroaryloxy, amino;
B is absent or selected from the group consisting of triazolyl, —NR⁷— or —N(NR⁷R⁸)—;
R⁷ and R⁸ are the same or different and are independently selected from the group consisting of hydrogen and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a ring structure which is unsubstituted or substituted with one, two or more R^{I}, for example, the ring structure is a 3- to 10-membered ring structure; or, R¹¹ and R¹², together with the atoms to which they are attached, form a ring structure which is unsubstituted or substituted with one, two or more R^{I}, for example, the ring structure is a 3- to 10-membered ring structure; or, R⁹ and R¹¹, together with the atoms to which they are attached, form a ring structure which is unsubstituted or substituted with one, two or more R^{I}, for example, the ring structure is a 3- to 10-membered ring structure; wherein any of the ring structures can be a monocyclic hydrocarbyl, bicyclic hydrocarbyl, heteromonocyclic hydrocarbyl, or heterobicyclic hydrocarbyl, each of which is unsubstituted or substituted with one, two or more R^{I}; for example, any of the ring structures can be the following group which is unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
each m is the same or different, and is independently selected from an integer of 0 to 10;
each n is the same or different, and is independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, aryloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
the wavy line represents the site attached to L;
provided that, when R⁶ is selected from the group consisting of alkyl, alkyloxy, aryl, arylalkyl, aryloxy, heteroaryl, heteroarylalkyl, heteroaryloxy or amino, each of which is unsubstituted or substituted with one, two or more R^{G}:
A is N; R¹ and R², together with the atoms to which they are attached, do not form a 5- to 10-membered ring structure which is unsubstituted or substituted with one, two or more R^{E}; and T^{L} is not a chemical bond when R⁷ is hydrogen;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl; and R^{A} is selected from the following groups which are unsubstituted or substituted with one, two or more R^{C}: cycloalkyl, cycloalkylalkyl, cycloalkyloxy;
or, B is —N(NR⁷R⁸)—;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl.

2. The conjugate, or a stereoisomer, racemate, tautomer, isotopologue, isotope-labeled compound, nitroxide or pharmaceutically acceptable salt thereof according to claim 1, wherein definition of each group in Formula (G) is independently selected from those listed in any of items (1a) to (1c):
(1a):
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, or heterobicyclic hydrocarbyl;
or R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, or heterobicyclic hydrocarbyl;
R⁴ is selected from the group consisting of alkyl, alkyloxy, halogen, hydroxyl, sulfhydryl, amino, cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from the group consisting of triazolyl, —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(N¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached with L, and * represents the site attached with B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocycloalkyl;
m and n are the same or different and are independently selected from an integer of 0 to 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
(1b):
A is selected from N or C—R^{A};
when A is N, R¹ is selected from the group consisting of hydrogen, hydroxyl, and the following groups which are unsubstituted or substituted with one, two or more R^{B}: alkyl, cycloalkyl, cycloalkylalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
when A is C—R^{A}, R^{A} and R¹ are the same or different and are independently selected from the group consisting of hydrogen, halogen, hydroxyl, sulfhydryl, cyano, nitro, and the following groups which are unsubstituted or substituted with one, two or more R^{C}: alkyl, cycloalkyl, cycloalkylalkyl, alkyloxy, cycloalkyloxy, alkyloxyalkyl, heterocyclyl, heterocyclylalkyl, aryl, arylalkyl, heteroaryl or heteroarylalkyl, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
or, R^{A} and R¹, together with the atom to which they are attached, form a 3- to 10-membered ring structure which is unsubstituted or substituted with one, two or more R^{D};
z is selected from 0 or 1;
R² is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
R³ is selected from the group consisting of hydrogen, halogen, and the following groups which are unsubstituted or substituted with one, two or more R^{E}: alkyl, alkyloxy, cycloalkyl, cycloalkylalkyl, cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of alkyl, alkyloxy, halogen, hydroxyl, amino and cyano;
R⁵ is selected from hydroxyl;
R⁶ is selected from the following groups which are unsubstituted or substituted with one, two or more R^{G}: cycloalkyl, cycloalkylalkyl, cycloalkyloxy, heterocyclyl, heterocyclylalkyl, heterocyclyloxy;
B is absent or selected from —NR⁷— or —N(NR⁷R⁸)—;
R⁷ and R⁸ are the same or different and are independently selected from the group consisting of hydrogen, and the following groups which are unsubstituted or substituted with one, two or more R^{H}: alkyl, cycloalkyl, cycloalkylalkyl, aryl, arylalkyl, heteroaryl, heteroarylalkyl, heterocyclyl, heterocyclylalkyl, HC(═O)—, alkyl-C(═O)—, cycloalkyl-C(═O)NH—, heterocyclyl-C(═O)NH—, aryl-C(═O)NH—, heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR³)*, #O—(CR⁹R¹⁰)ₘ—(CR¹¹R¹²)—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of hydrogen, halogen, cyano, and the following groups which are unsubstituted or substituted with one, two or more R^{I}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹² together with the atoms to which they are attached form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: alkyl, cycloalkyl, cycloalkylalkyl, aryl, arylalkyl, heteroaryl, heteroarylalkyl, heterocyclyl, heterocyclylalkyl;
m and n are the same or different, and are independently selected from an integer of 0 to 10;
each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{K} is the same or different, and is independently selected from the group consisting of deuterium, halogen, hydroxyl, cyano, nitro, and the following groups which are unsubstituted or substituted with one, two or more R^{L}: alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
each R^{L} is the same or different and is independently selected from the group consisting of deuterium, halogen, hydroxyl, cyano, nitro, alkyl, alkyloxy, cycloalkyl, cycloalkylalkyl, cycloalkyloxy, aryl, arylalkyl, aryloxy, heteroaryl, heteroarylalkyl, heteroaryloxy, heterocyclyl, heterocyclylalkyl, heterocyclyloxy, NH₂, HC(═O)NH—, alkyl-C(═O)NH—, cycloalkyl-C(═O)NH—, heterocyclyl-C(═O)NH—, aryl-C(═O)NH—, heteroaryl-C(═O)NH—;
(1c):
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
or, R^{A} and R¹, together with the atom to which they are attached, form a 3- to 8-membered ring structure which is unsubstituted or substituted with one, two or more R^{D}, for example, the 3- to 8-membered ring structure is 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy;
preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C_{3 -6} cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

3-4. (canceled)

5. The conjugate, or a stereoisomer, racemate, tautomer, isotopologue, isotope-labeled compound, nitroxide or pharmaceutically acceptable salt thereof according to claim 1, wherein definition of each group in Formula (G) is independently selected from those listed in items (1d) and (1e):
(1d):
A is selected from N or C—R^{A};
when A is N, R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₁₀ alkyl, C₃₋₁₀ cycloalkyl, C₃₋₁₀ cycloalkyl-C₁₋₁₀ alkyl, C₁₋₁₀ alkyloxy, C₃₋₁₀ cycloalkyloxy, C₁₋₁₀ alkyloxy-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is C—R^{A}, R^{A} and R¹ are the same or different and are independently selected from the group consisting of ¹H, ²H, halogen, hydroxyl, sulfhydryl, deuterated hydroxyl, deuterated sulfhydryl, cyano, nitro, and the following groups which are unsubstituted or substituted with one, two or more R^{C}: C₁₋₆ alkyl, C₃₋₆ cycloalkyl, C₃₋₆ cycloalkyl-C₁₋₆ alkyl, C₁₋₆ alkyloxy, C₃₋₆ cycloalkyloxy, C₁₋₆ alkyloxy-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
or, R^{A} and R¹, together with the atom to which they are attached, form a 3- to 8-membered ring structure which is unsubstituted or substituted with one, two or more R^{D}, for example, the 3- to 8-membered ring structure is a 3-, 4-, 5-, 6-, 7- or 8-membered ring structure;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
provided that, R¹, R² and the atoms to which they are attached, or R², R³ and the atoms to which they are attached, at least one of these two sets of groups together form a 4- to 10-membered ring structure which is unsubstituted or substituted with one, two or more R^{E}, and the 4- to 10-membered ring structure can be selected from, for example, the group consisting of 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is hydroxyl;
R⁶ is selected from the following groups which are unsubstituted or substituted with one, two or more R^{G}: C₁₋₁₀ alkyl, C₁₋₁₀ alkyloxy, C₆₋₁₀ aryl, C₆₋₁₀ arylalkyl, C₆₋₁₀ aryloxy, 5- to 6-membered heteroaryl, 5- to 6-membered heteroarylalkyl, 5- to 6-membered heteroaryloxy;
B is absent or selected from the group consisting of triazolyl, —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different, and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆-10 aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; wherein, any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, amino, nitro and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different, and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different, and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—;
(1e):
A is N;
R¹ is selected from the group consisting of ¹H, ²H, hydroxyl, deuterated hydroxyl, and the following groups which are unsubstituted or substituted with one, two or more R^{B}: C₁₋₁₀ alkyl, C₃₋₁₀ cycloalkyl, C₃₋₁₀ cycloalkyl-C₁₋₁₀ alkyl, 3- to 10-membered heterocyclyl, 3- to 10-membered heterocyclyl-C₁₋₁₀ alkyl, C₆₋₁₀ aryl, C₆₋₁₀ aryl-C₁₋₁₀ alkyl, 5- to 10-membered heteroaryl or 5- to 10-membered heteroaryl-C₁₋₁₀ alkyl, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, when A is N, R¹ is selected from the group consisting of ¹H, ²H, hydroxyl, deuterated hydroxyl, and the following groups which are unsubstituted or substituted with one, two or more R^{B}: C₁₋₆ alkyl, C₃₋₆ cycloalkyl, C₃₋₆ cycloalkyl-C₁₋₆ alkyl, 3- to 6-membered heterocyclyl, 3- to 6-membered heterocyclyl-C₁₋₆ alkyl, phenyl, phenyl-C₁₋₆ alkyl, 5- to 6-membered heteroaryl or 5- to 6-membered heteroaryl-C₁₋₆ alkyl, NH₂, HC(═O)NH—, C₁₋₆ alkyl-C(═O)NH—, C₃₋₆ cycloalkyl-C(═O)NH—, 3- to 6-membered heterocyclyl-C(═O)NH—, phenyl-C(═O)NH—, 5- to 6-membered heteroaryl-C(═O)NH—;
z is selected from 0 or 1;
R² is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R² is selected from the group consisting of ¹H, ²H, hydrogen, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy;
R³ is selected from the group consisting of ¹H, ²H, halogen, and the following groups which are unsubstituted or substituted with one, two or more R^{E}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy; preferably, R³ is selected from the group consisting of ¹H, ²H, halogen, and the following groups which are unsubstituted or substituted with one, two or more R^{E}: C₁₋₆ alkyl, C₁₋₆ alkyloxy, C₃₋₆ cycloalkyl, C₃₋₆ cycloalkyl-C₁₋₁₀ alkyl, C₃₋₆ cycloalkyloxy;
or, R¹ and R², together with the atoms to which they are attached, form a 5- to 10-membered ring structure which is unsubstituted or substituted with one, two or more R^{E}, and the 5- to 10-membered ring structure can be selected from, for example, the group consisting of 5-, 6-, 7-, 8-, 9- or 10-membered monocyclic hydrocarbyl, bicyclic hydrocarbyl, heteromonocyclic hydrocarbyl, heterobicyclic hydrocarbyl;
or, R² and R³, together with the atoms to which they are attached, form a 4- to 10-membered ring structure which is unsubstituted or substituted with one, two or more R^{F}, and the 4- to 10-membered ring structure can be selected from, for example, the group consisting of 4, 5-, 6-, 7-, 8-, 9- or 10-membered monocyclic hydrocarbyl, bicyclic hydrocarbyl, heteromonocyclic hydrocarbyl, heterobicyclic hydrocarbyl;
R⁴ is selected from the group consisting of C₁₋₁₀ alkyl, C₁₋₁₀ alkyloxy, halogen, hydroxyl, sulfhydryl, amino, cyano; preferably, R⁴ is selected from the group consisting of C₁₋₆ alkyl, C₁₋₆ alkyloxy, halogen, hydroxyl, sulfhydryl, amino, cyano;
R⁵ is hydroxyl;
R⁶ is selected from the following groups which are unsubstituted or substituted with one, two or more R^{G}: C₃₋₁₀ cycloalkyl, C₃₋₁₀ cycloalkyl-C₁₋₁₀ alkyl, C₃₋₁₀ cycloalkyloxy, 3- to 10-membered heterocyclyl, 3- to 10-membered heterocyclyl-C₁₋₁₀ alkyl, 3- to 10-membered heterocyclyloxy;
preferably, R⁶ is selected from the following groups which are unsubstituted or substituted with one, two or more R^{G}: C₃₋₆ cycloalkyl, C₃₋₆ cycloalkyl-C₁₋₆ alkyl, C₃₋₆ cycloalkyloxy, 3- to 6-membered heterocyclyl, 3- to 6-membered heterocyclyl-C₁₋₆ alkyl, 3- to 6-membered heterocyclyloxy;
B is selected from —NR⁷— or —N(NR⁷R⁸)—;
R⁷ and R⁸ are the same or different and are independently selected from the group consisting of ¹H, ²H, and the following groups which are unsubstituted or substituted with one, two or more R^{H}C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl, HC(═O)—, C₁₋₁₀ alkyl-C(═O)—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, R⁷ and R⁸ are the same or different and are independently selected from the group consisting of hydrogen, and the following groups which are unsubstituted or substituted with one, two or more R^{H}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl, HC(═O)—, C₁₋₆ alkyl-C(═O)—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
T^{L} is selected from the group consisting of chemical bond, #O—(CR⁹R¹⁰)ₘ—(CR¹¹R¹²)ₙ—C(═O)*, #O—(CR⁹R¹⁰)ₘ—(CR¹¹R¹²)ₙ*, #N(R¹³)—(CR⁹R¹⁰)ₘ—(CR¹¹R¹²)ₙ—C(O)*, #S—(CR⁹R¹⁰)ₘ—(CR¹¹R¹²)ₙ—C(O)*, #O—(CR⁹R¹⁰)ₘ—(CR¹¹R¹²)ₙ—C(NR¹³)*, #O—(CR⁹R¹⁰)ₘ—(CR¹¹R¹²)ₙ—P(═O)(OR¹⁴)*, *O—(CR⁹R¹⁰)ₘ—(CR¹¹R¹²)ₙ—C(═O) #, *O—(CR⁹R¹⁰)ₘ—(CR¹¹R¹²)ₙ #, *N(R¹³)—(CR⁹R¹⁰)ₘ—(CR¹¹R¹²)ₙ—C(O) #, *S—(CR⁹R¹⁰)ₘ—(CR¹¹R¹²)ₙ—C(O) #, *O—(CR⁹R¹⁰)ₘ—(CR¹¹R¹²)ₙ—C(NR¹³) #, *O—(CR⁹R¹⁰)ₘ—(CR¹¹R¹²)ₙ—P(═O)(OR¹⁴) #, wherein # represents the site attached to L, and * represents the site attached to B;
each of R⁹, R¹⁰, R¹¹ and R¹² is the same or different and is independently selected from the group consisting of ¹H, ²H, halogen, cyano, and the following groups which are unsubstituted or substituted with one, two or more R^{I}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy; preferably, each of R⁹, R¹⁰, R¹¹ and R¹² is the same or different and is independently selected from the group consisting of hydrogen, halogen, cyano, and the following groups which are unsubstituted or substituted with one, two or more R^{I}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆-10 aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₁₀ alkyl, 3- to 6-membered heterocyclyloxy;
or, R⁹ and R¹⁰, together with the atoms to which they are attached, form a 3- to 10-membered ring structure which is unsubstituted or substituted with one, two or more R^{I}; or, R¹¹ and R¹², together with the atoms to which they are attached, form a 3- to 10-membered ring structure which is unsubstituted or substituted with one, two or more R^{I}; or, R⁹ and R¹¹, together with the atoms to which they are attached, form a 3- to 10-membered ring structure which is unsubstituted or substituted with one, two or more R^{I}; any of the 3- to 10-membered ring structures can be selected from, for example, the following groups which are unsubstituted or substituted with one, two or more R^{I}: 3-, 4-, 5-, 6-, 7-, 8-, 9- or 10-membered monocyclic hydrocarbyl, bicyclic hydrocarbyl, heteromonocyclic hydrocarbyl, heterobicyclic hydrocarbyl;
each R¹³ is the same or different and is independently selected from the following groups which are unsubstituted or substituted with one, two or more R^{J}: C₁₋₁₀ alkyl, C₃₋₁₀ cycloalkyl, C₃₋₁₀ cycloalkyl-C₁₋₁₀ alkyl, C₆₋₁₀ aryl, C₆₋₁₀ aryl-C₁₋₁₀ alkyl, 5- to 10-membered heteroaryl, 5- to 10-membered heteroaryl-C₁₋₁₀ alkyl, 3- to 10-membered heterocyclyl, 3- to 10-membered heterocyclyl-C₁₋₁₀ alkyl; preferably, each R¹³ is the same or different and is independently selected from the following groups which are unsubstituted or substituted with one, two or more R^{J}: C₁₋₆ alkyl, C₃₋₆ cycloalkyl, C₃₋₆ cycloalkyl-C₁₋₆ alkyl, C₆₋₁₀ aryl, C₆₋₁₀ aryl-C₁₋₆ alkyl, 5- to 6-membered heteroaryl, 5- to 6-membered heteroaryl-C₁₋₆ alkyl, 3- to 6-membered heterocyclyl, 3- to 6-membered heterocyclyl-C₁₋₆ alkyl;
m and n are the same or different and are independently selected from an integer of 0 to 10, such as 0, 1, 2, 3, 4, 5, 6, 7, 8, 9 or 10;
each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each of R^{B}, R^{C}, R^{D}, R^{E}, R^{F}, R^{G}, R^{H}, R^{I} and R^{J} is the same or different and is independently selected from the group consisting of deuterium, halogen, hydroxyl, cyano, nitro, and the following groups which are unsubstituted or substituted with one, two or more R^{K}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{K} is the same or different and is independently selected from the group consisting of deuterium, halogen, hydroxyl, cyano, nitro, and the following groups which are unsubstituted or substituted with one, two or more R^{L}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—; preferably, each R^{K} is the same or different and is independently selected from the group consisting of deuterium, halogen, hydroxyl, cyano, nitro, and the following groups which are unsubstituted or substituted with one, two or more R^{L}: C₁₋₆ alkyl, C₁₋₆ alkyloxy, C₃₋₆ cycloalkyl, C₃₋₆ cycloalkyl-C₁₋₆ alkyl, C₃₋₆ cycloalkyloxy, C₆₋₁₀ aryl, C₆₋₁₀ aryl-C₁₋₆ alkyl, C₆₋₁₀ aryloxy, 5- to 6-membered heteroaryl, 5- to 6-membered heteroaryl-C₁₋₆ alkyl, 5- to 6-membered heteroaryloxy, 3- to 6-membered heterocyclyl, 3- to 6-membered heterocyclyl-C₁₋₆ alkyl, 3- to 6-membered heterocyclyloxy, NH₂, HC(═O)NH—, C₁₋₆ alkyl-C(═O)NH—, C₃₋₆ cycloalkyl-C(═O)NH—, 3- to 6-membered heterocyclyl-C(═O)NH—, C₆₋₁₀ aryl-C(═O)NH—, 5- to 6-membered heteroaryl-C(═O)NH—;
each R^{L} is the same or different and is independently selected from the group consisting of deuterium, halogen, hydroxyl, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, C₆₋₁₀ aryl, C₆₋₁₀ aryl-C₁₋₁₀ alkyl, C₆₋₁₀ aryloxy, 5- to 10-membered heteroaryl, 5- to 10-membered heteroaryl-C₁₋₁₀ alkyl, 5- to 10-membered heteroaryloxy, 3- to 10-membered heterocyclyl, 3- to 10-membered heterocyclyl-C₁₋₁₀ alkyl, 3- to 10-membered heterocyclyloxy, NH₂, HC(═O)NH—, C₁₋₁₀ alkyl-C(═O)NH—, C₃₋₁₀ cycloalkyl-C(═O)NH—, 3- to 10-membered heterocyclyl-C(═O)NH—, C₆₋₁₀ aryl-C(═O)NH—, 5- to 10-membered heteroaryl-C(═O)NH—.

6. (canceled)

7. The conjugate, or a stereoisomer, racemate, tautomer, isotopologue, isotope-labeled compound, nitroxide or pharmaceutically acceptable salt thereof according to claim 1, wherein:
R¹ and R², together with the quinolyl to which they are connected, form one of the following substructures, wherein the substructures may be unsubstituted or substituted with one, two or more R^{E}: 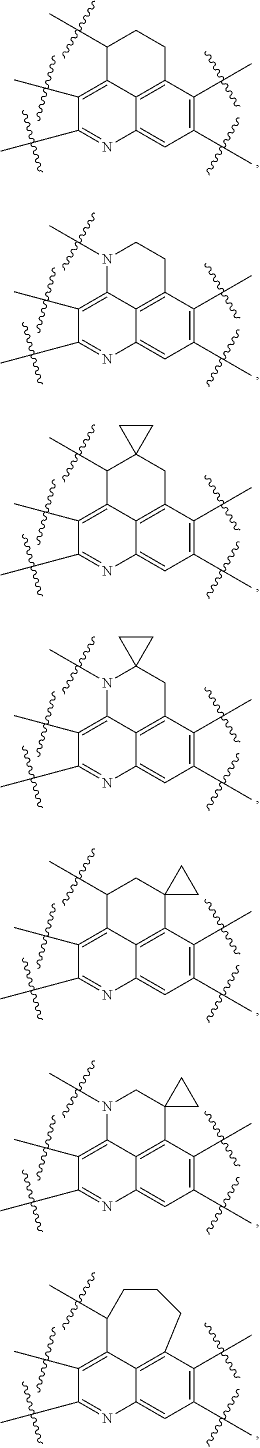 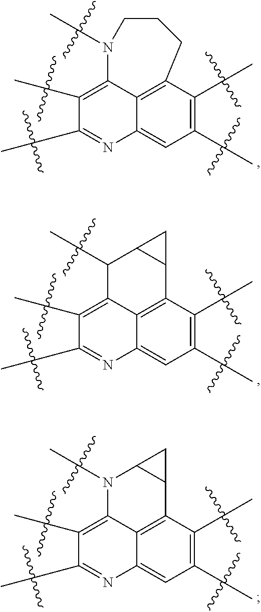 [SMILES: *c1cc2nc(*)c(*)c3c2c(c1*)CCCN3* ; *c1cc2nc(*)c(*)c3c2c(c1*)C1CC1C3* ; *c1cc2nc(*)c(*)c3c2c(c1*)C1CC1N3*]
or, R² and R³, together with the quinolyl to which they are connected, form one of the following substructures, wherein the substructures may be unsubstituted or substituted with one, two or more R^{F}: 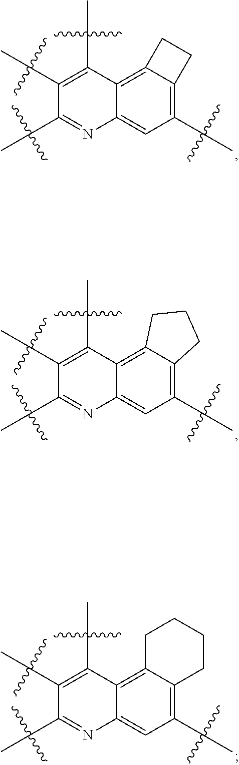 [SMILES: *c1cc2nc(*)c(*)c(*)c2c2c1CC2 ; *c1cc2nc(*)c(*)c(*)c2c2c1CCC2 ; *c1cc2nc(*)c(*)c(*)c2c2c1CCCC2]
or preferably, B is absent or selected from the group consisting of 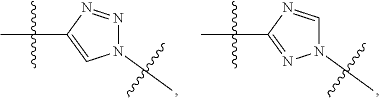 [SMILES: *c1cn(*)nn1 ; *c1ncn(*)n1]
—NH— or —N(NR⁷R⁸)—, wherein R⁷ and R⁸ independently have the definitions as described in claim 1.

8. The conjugate, or a stereoisomer, racemate, tautomer, isotopologue, isotope-labeled compound, nitroxide or pharmaceutically acceptable salt thereof according to claim 1, wherein G is selected from the group represented by the following Formulas (G-1), (G-2) and (G-3):
Formula (G-1): 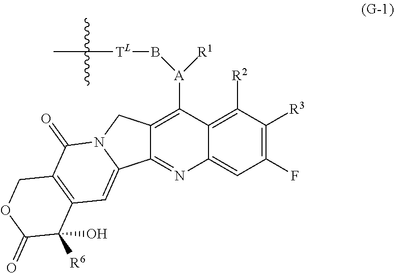
wherein, A, B, T^{L}, R¹, R², R³, and R⁶ independently have the definitions as described in claim 1; Formula (G-2): 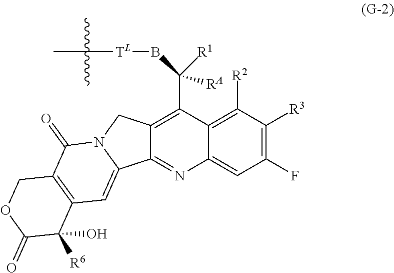
wherein, R^{A}, B, T^{L}, R¹, R², R³, and R⁶ have the definitions as described in claim 1;
Formula (G-3): 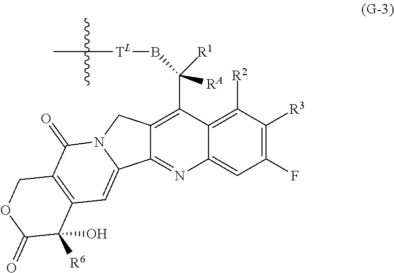
wherein, R^{A}, B, T^{L}, R¹, R², R³, and R⁶ have the definitions as described in claim 1;
or, G is selected from the following groups or the groups formed by connecting -T^{L}- with the following group, at the wavy line of the following group: 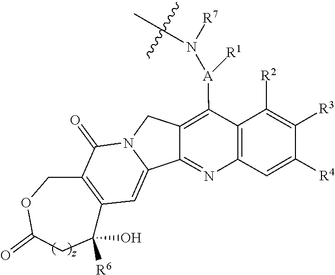
wherein, A, R¹, R², R³, R⁴, R⁶, R⁷, and z have the definitions as described in claim 1; 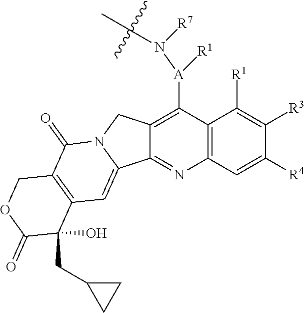
wherein, A, R¹, R², R³, R⁴, and R⁷ have the definitions as described in claim 1; 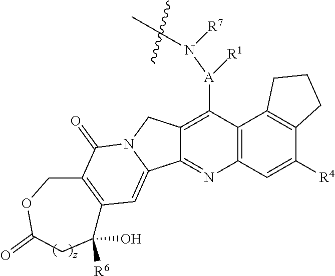
wherein, A, R¹, R⁴, R⁶, R⁷, and z have the definitions as described in claim 1;
particularly, G is selected from the following groups or the groups formed by connecting -T^{L}- with the following group, at the wavy line of the following groups:

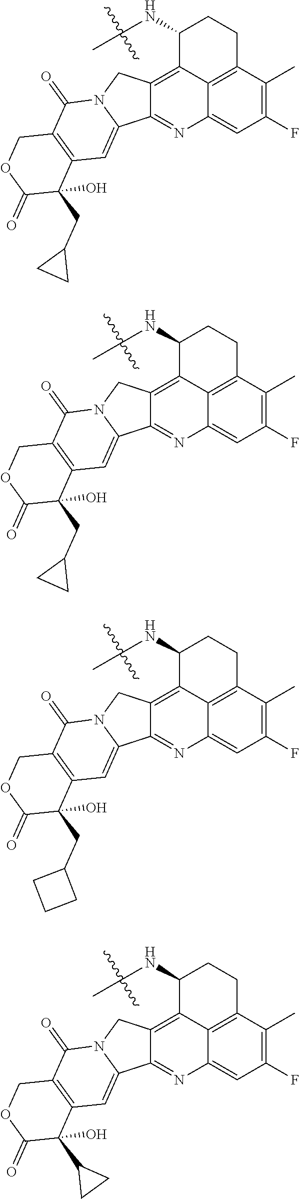 [SMILES: *N[C@@H]1CCc2c(C)c(F)cc3nc4c(c1c23)Cn1c-4cc2c(c1=O)COC(=O)[C@]2(O)CC1CC1 ; *N[C@H]1CCc2c(C)c(F)cc3nc4c(c1c23)Cn1c-4cc2c(c1=O)COC(=O)[C@]2(O)CC1CC1 ; *N[C@H]1CCc2c(C)c(F)cc3nc4c(c1c23)Cn1c-4cc2c(c1=O)COC(=O)[C@]2(O)CC1CCC1 ; *N[C@H]1CCc2c(C)c(F)cc3nc4c(c1c23)Cn1c-4cc2c(c1=O)COC(=O)[C@]2(O)C1CC1]

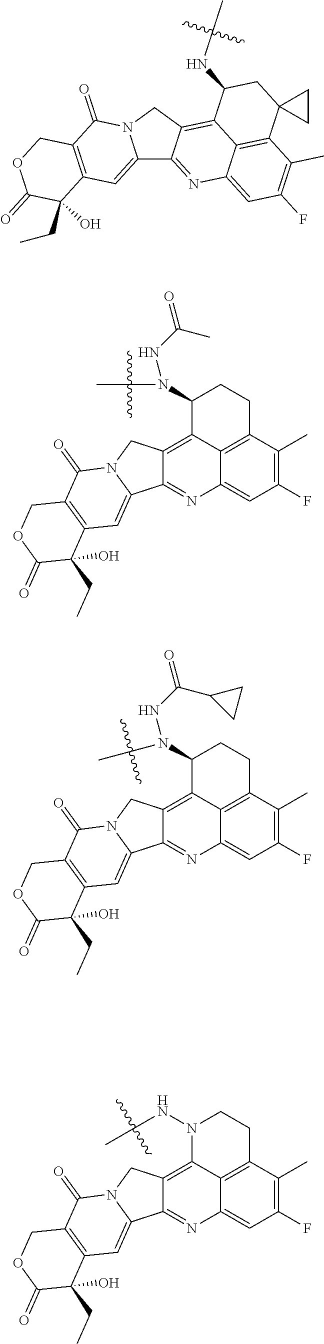 [SMILES: *N[C@H]1CC2(CC2)c2c(C)c(F)cc3nc4c(c1c23)Cn1c-4cc2c(c1=O)COC(=O)[C@]2(O)CC ; *N(NC(C)=O)[C@H]1CCc2c(C)c(F)cc3nc4c(c1c23)Cn1c-4cc2c(c1=O)COC(=O)[C@]2(O)CC ; *N(NC(=O)C1CC1)[C@H]1CCc2c(C)c(F)cc3nc4c(c1c23)Cn1c-4cc2c(c1=O)COC(=O)[C@]2(O)CC ; *NN1CCc2c(C)c(F)cc3nc4c(c1c23)Cn1c-4cc2c(c1=O)COC(=O)[C@]2(O)CC]

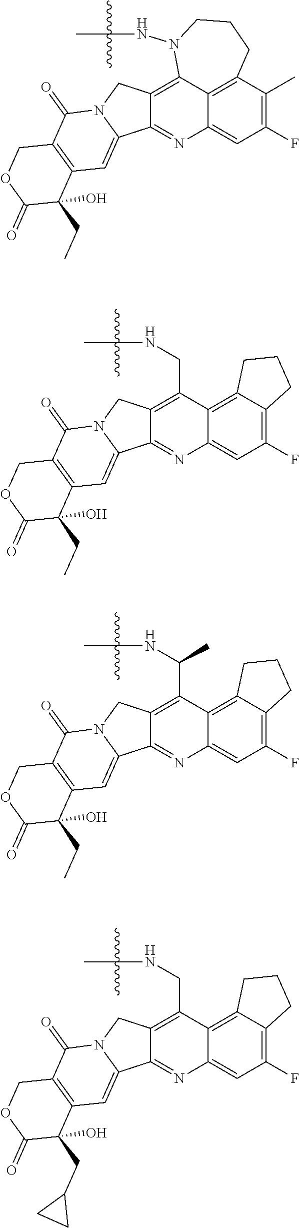 [SMILES: *NN1CCCc2c(C)c(F)cc3nc4c(c1c23)Cn1c-4cc2c(c1=O)COC(=O)[C@]2(O)CC ; *NCc1c2c(nc3cc(F)c4c(c13)CCC4)-c1cc3c(c(=O)n1C2)COC(=O)[C@]3(O)CC ; *N[C@@H](C)c1c2c(nc3cc(F)c4c(c13)CCC4)-c1cc3c(c(=O)n1C2)COC(=O)[C@]3(O)CC ; *NCc1c2c(nc3cc(F)c4c(c13)CCC4)-c1cc3c(c(=O)n1C2)COC(=O)[C@]3(O)CC1CC1]

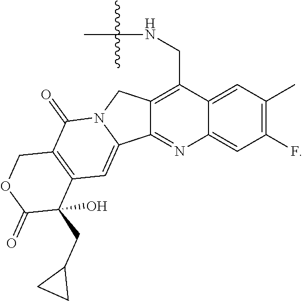 [SMILES: *NCc1c2c(nc3cc(F)c(C)cc13)-c1cc3c(c(=O)n1C2)COC(=O)[C@]3(O)CC1CC1]

9-13. (canceled)

14. The conjugate, or a stereoisomer, racemate, tautomer, isotopologue, isotope-labeled compound, nitroxide or pharmaceutically acceptable salt thereof according to claim 1, wherein L is selected from a chemical bond or a linker represented by the Formula (L) as defined below: 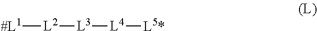
wherein, L¹ is a linking moiety to the targeting moiety Tp, formed from a reactive group L^{1′} through its reaction with the targeting moiety Tp, and # represents the site attached to the targeting moiety Tp;
for example, L^{1′} is a maleimide group, then L¹ is the following structure: 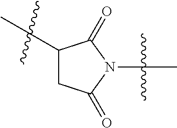 [SMILES: *C1CC(=O)N(*)C1=O]
or its ring-opening form: 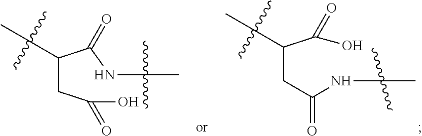 [SMILES: *NC(=O)C(*)CC(=O)O ; *NC(=O)CC(*)C(=O)O]
L² is absent or a spacer between L¹ and L³;
L³ is a peptide moiety;
L⁴ is absent or a spacer between the peptide moiety and L⁵;
L⁵ is a linking moiety between L⁴ and a bioactive molecule G, formed from a reactive group L^{5′} through its reaction with the bioactive molecule G or intermediates thereof, and * represents the site attached to the bioactive molecule G;
optionally:
the following hydrophilic moiety or steric hindrance moiety is inserted between the above moieties of L, preferably between L¹ and L², or between L² and L³, or is inserted by replacing L²:
hydrophilic moiety: 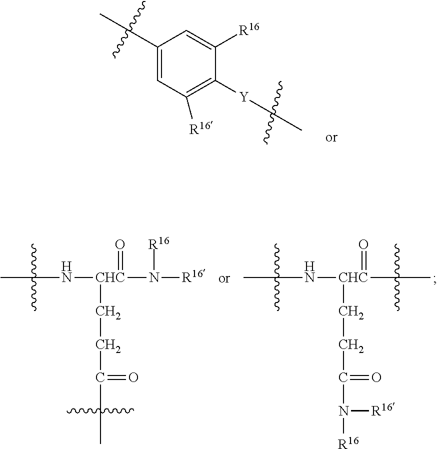
steric hindrance moiety: 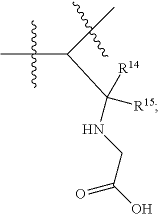
R¹⁶ and R^{16′} are the same or different, and at least one of them is selected from a hydrophilic group, and the other is selected from the following substituents: hydrogen, halogen, cyano, amino, nitro, and the following groups which are unsubstituted or substituted with one, two or more R^{zg}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, aminocarbonyl;
the hydrophilic group is selected from a polyethylene glycol group, a C₁₋₁₀ alkyl substituted with 1 to 10 hydroxyl groups, or a sugar ring-containing group, or C₁₋₆ alkyl-NHCO— (e.g., C₁₋₄ alkyl-NHCO—), preferably a polyethylene glycol group, more preferably —C(═O)—NH—(CH₂CH₂O)ₚ—C₁₋₁₀ alkyl or —NH—(CH₂CH₂O)ₚ—C₁₋₁₀ alkyl, or preferably a C₁₋₁₀ alkyl substituted with 1 to 10 hydroxyl groups, more preferably 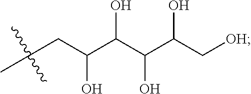 [SMILES: *CC(O)C(O)C(O)C(O)CO]
each p is the same or different, and is independently selected from an integer of 0 to 10, preferably 1, 2, 3, 4, 5, 6, 7 or 8;
Y is selected from the group consisting of O, S, C₁₋₁₀ alkylene, wherein 1, 2 or 3 of the methylene groups of the alkylene can be optionally replaced by 0 or S;
R¹⁴ and R¹⁵ are the same or different and are independently selected from the group consisting of hydrogen, halogen, cyano, amino, nitro, and the following groups which are unsubstituted or substituted with one, two or more R^{zh}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy;
or, R¹⁴ and R¹⁵, together with the atom to which they are attached, form a C₃₋₁₀ cycloalkyl which is unsubstituted or substituted with one, two or more R^{zh};
each of R^{zg} and R^{zh} is the same or different and is independently selected from the group consisting of halogen, hydroxyl, amino, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy;
the hydrophilic moiety or steric hindrance moiety as defined above is present or absent;
particularly, L¹ is formed from any of L^{1′} through its reaction with the targeting moiety Tp, L^{1′} is preferably a sulfhydryl-reactive group, an amine-reactive group, a carboxyl-reactive group, a proline residue-reactive group, a tyrosine residue-reactive group, a disulfide bridge-containing group; for an antibody introduced with a non-natural amino acid, it can also be selected from reactive groups in click chemistry such as ketone, azide, alkyne, cyclopropene or diene;
L^{1′} is preferably a sulfhydryl-reactive group;
L^{1′} is further preferably a maleimide group or a substituted maleimide group, and L¹-L² preferably has the following structure:
a fragment that is prepared from (N-maleimidomethyl)-carboxylic acid-N-hydroxysuccinimide ester, and has the structure as follows: 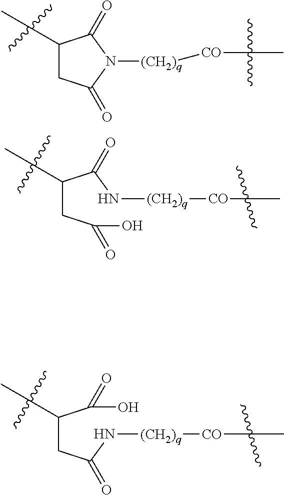
(q is an integer of 0 to 10, preferably 1, 2, 3, 4, 5, 6, 7 or 8);
or a fragment that is prepared from (meta)-maleimidobenzoic acid N-hydroxysuccinimide ester (MBS), and has the structure as follows: 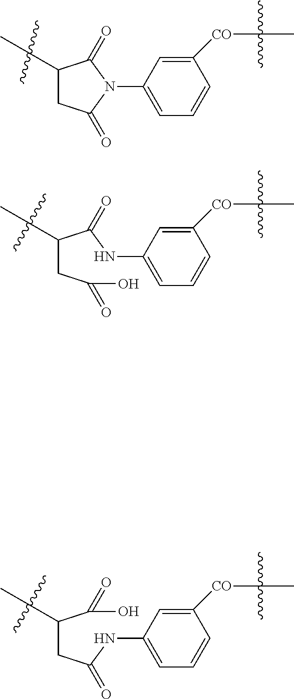 [SMILES: *C(=O)c1cccc(N2C(=O)CC(*)C2=O)c1 ; *C(=O)c1cccc(NC(=O)C(*)CC(=O)O)c1 ; *C(=O)c1cccc(NC(=O)CC(*)C(=O)O)c1]
or a fragment that is prepared from 4-(N-maleimidomethyl)-cyclohexane-1-carboxylic acid N-hydroxysuccinimide ester (SMCC), and has the structure as follows: 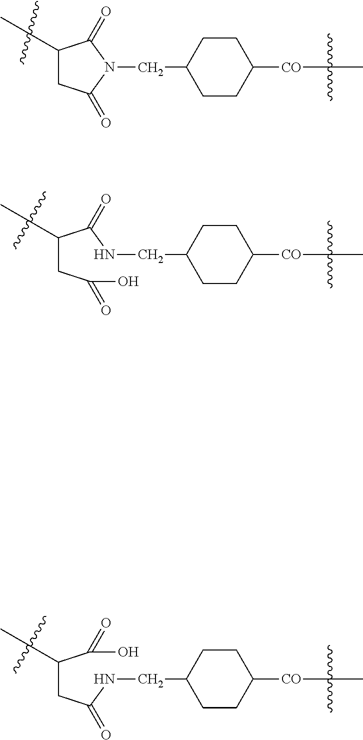 [SMILES: *C(=O)C1CCC(CN2C(=O)CC(*)C2=O)CC1 ; *C(=O)C1CCC(CNC(=O)C(*)CC(=O)O)CC1 ; *C(=O)C1CCC(CNC(=O)CC(*)C(=O)O)CC1]
or L^{1′} is preferably a sulfhydryl-reactive group having the following structure:
Hal-Het-
Hal is selected from the group consisting of halogen, OMs, OTs, OTf, nitro, and the following groups which are optionally substituted with one or more R^{z6}: alkyl thioether group, aryl thioether group, heteroaryl thioether group, alkyl sulfoxide group, aryl sulfoxide group, heteroaryl sulfoxide group, alkyl sulfonyl group, aryl sulfonyl group, heteroaryl sulfonyl group; wherein R^{z6} is independently selected from the group consisting of H (hydrogen), D (deuterium), halogen, CN, nitro, C₁₋₆ alkyl, halogenated C₁₋₆ alkyl, C₁₋₆ alkoxy, 6- to 10-membered aryl and 5- to 12-membered heteroaryl;
Het is selected from 5- to 10-membered heteroaryl which is optionally substituted with one or more R^{z7}; wherein R^{z7} is independently selected from the group consisting of H (hydrogen), D (deuterium), halogen, CN, nitro, C₁₋₄ alkyl and halogenated C₁₋₄ alkyl;
in a preferred embodiment, Hal is preferably mesyl, and Het is preferably pyrimidinyl;
in a preferred embodiment, Hal-Het- is: 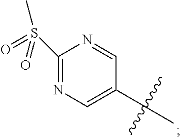 [SMILES: *c1cnc(S(C)(=O)=O)nc1]
the corresponding L¹ has the structure as follows: 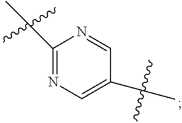 [SMILES: *c1cnc(*)nc1]
and L^{1′}-L² preferably has the structure as follows: 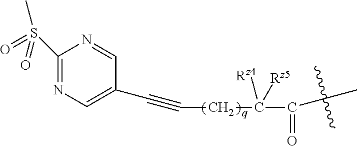
q is an integer selected from 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
further preferably has the structure as follows:

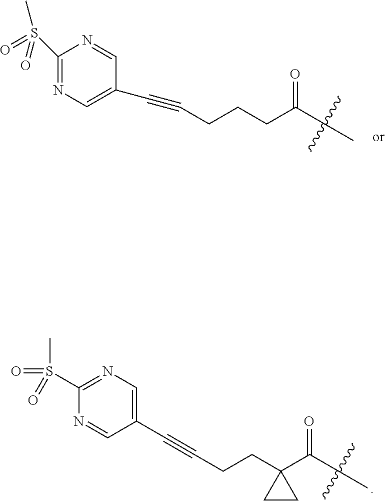 [SMILES: *C(=O)CCCC#Cc1cnc(S(C)(=O)=O)nc1 ; *C(=O)C1(CCC#Cc2cnc(S(C)(=O)=O)nc2)CC1]

15. (canceled)

16. The conjugate, or a stereoisomer, racemate, tautomer, isotopologue, isotope-labeled compound, nitroxide or pharmaceutically acceptable salt thereof according to claim 14, wherein
L² is absent or selected from the group consisting of C₁₋₁₀ alkylene, C₂₋₁₀ alkenylene, C₂₋₁₀ alkynylene, C₃₋₁₀ cycloalkyl, C₆₋₁₂ aryl or 6- to 12-membered heteroaryl or a combination of the above fragments, and is optionally interrupted by a carbonyl group, O, S, or N, and optionally substituted with C₁₋₆ alkyl, C₃₋₆ cycloalkyl, halogen, halogenated C₁₋₆ alkyl, and optionally, the alkyl or halogenated alkyl, together with the C atom to which it is attached, forms a C₃₋₆ cycloalkyl, L² is connected to L¹ or L³ through any functional group or covalent bond;
preferably, L² is connected to the N-terminal of the peptide moiety through —C(R^{z4}R^{z5})—CO—;
preferably, R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
further preferably, L² is —(CH₂)_{q}—, —(CH₂)_{q}—C(═O)—, or —(C≡C)—(CH₂)_{q}—C(R^{z4}R^{z5})C(═O)—, wherein q is an integer of 0 to 10, preferably 1, 2, 3, 4, 5, 6, 7 or 8;
preferably, R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
or,
L³ is selected from a divalent peptide group comprising 2 to 8 optionally substituted natural amino acid residues or optionally substituted non-natural amino acid residues, each of the amino acid residues is the same or different and is independently selected from the following amino acid residues: alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucine (Nle), selenocysteine (Sec), pyrrolysine (Pyl), homoserine, homocysteine, demethylpyrrolysine.
further preferably, L³ is selected from a divalent peptide group composed of 2, 3, 4, 5 or 6 optionally substituted natural amino acid residues or optionally substituted non-natural amino acid residues, each of the amino acid residues is the same or different and is independently selected from the following amino acid residues: alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucine (Nle), selenocysteine (Sec), pyrrolysine (Pyl), homoserine, homocysteine, demethylpyrrolysine; for example, -ValCit-; -CitVal-; -AlaAla-; -AlaCit-; -CitAla-; -AsnCit-; -CitAsn-; -CitCit-; -ValGlu-; -GluVal-; -SerCit-; -CitSer-; -LysCit-; -CitLys-; -AspCit-; -CitAsp-; -AlaVal-; -ValAla-; -PheAla-; -AlaPhe-; -PheLys-; -LysPhe-; -ValLys-; -LysVal-; -AlaLys-; -LysAla-; -PheCit-; -CitPhe-; -LeuCit-; -CitLeu-; -IleCit-; -CitIle-; -PheArg-; -ArgPhe-; -CitTrp-; -TrpCit-; -PhePheLys-; -LysPhePhe-; -DPhePheLys-; -DLysPhePhe-; -GlyPheLys-; -LysPheGly-; -GlyPheLeuGly-; -GlyLeuPheGly-; -AlaLeuAlaLeu-; -GlyGlyGly-; -GlyGlyGlyGly-; -GlyPheValGly-; -GlyValPheGly-; -GlyGlyPheGly-; -AlaAlaAla-; most preferably, L³ is -GlyGlyPheGly-.

17. (canceled)

18. The conjugate, or a stereoisomer, racemate, tautomer, isotopologue, isotope-labeled compound, nitroxide or pharmaceutically acceptable salt thereof according to claim 14, wherein
L⁴ is preferably a group with self-cleaving properties, and a self-cleaving group or self-immolative group is a group that initiates drug release through an intramolecular reaction such as 1,4-elimination, 1,6-elimination or cyclization elimination independent of the action of an enzyme;
L⁵ is formed from any reactive group L^{5′} through its reaction with a bioactive molecule or intermediate thereof, wherein L^{5′} is preferably a carboxylic acid group or an active ester group, which reacts with OH, SH or NH or NH₂ in the bioactive molecule to form L⁵ with the structure of: —C(O)O—, —C(O)S—, —C(O)N— or —C(O)NH— (including the atom of O, S or N in the bioactive molecule);
L⁴-L⁵ is preferably:
a PABC spacer arm with the structure as follows: 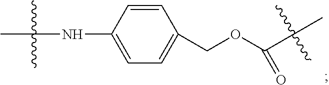 [SMILES: *Nc1ccc(COC(*)=O)cc1]
a GABA spacer arm with the structure as follows: 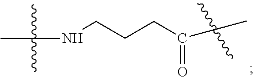 [SMILES: *NCCCC(*)=O]
an α,α-dimethyl GABA spacer arm with the structure as follows: 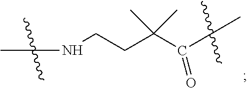 [SMILES: *NCCC(C)(C)C(*)=O]
or a β,β-dimethyl GABA spacer arm with the structure as follows: 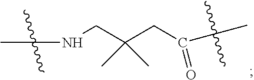 [SMILES: *NCC(C)(C)CC(*)=O]
most preferably, L⁴-L⁵ is: —NR^{z1}—C(R^{z2}R^{z3})-T^{L};
wherein:
R^{z1} is H or C₁₋₄ alkyl;
R^{z2} and R^{z3} are the same or different and are independently selected from H or C₁₋₄ alkyl;
T^{L} has the definition as described in claim 1, and one of T^{L} in L⁴-L⁵ or G is a chemical bond.

19. The conjugate, or a stereoisomer, racemate, tautomer, isotopologue, isotope-labeled compound, nitroxide or pharmaceutically acceptable salt thereof according to claim 14, wherein
L¹ is generated by coupling Tp with a sulfhydryl-reactive group contained in maleimide group, a substituted maleimide group or Hal-Het-;
the following hydrophilic moiety is inserted between L¹ and L², or between L² and L³, or is inserted by replacing L²:
hydrophilic moiety: 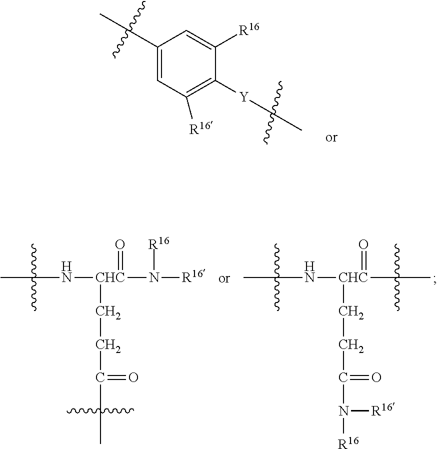
or,
L¹ is generated by coupling Tp with a sulfhydryl-reactive group contained in maleimide group or a substituted maleimide group;
the following steric hindrance moiety is inserted between L¹ and L², or between L² and L³, or is inserted by replacing L²:
steric hindrance moiety:

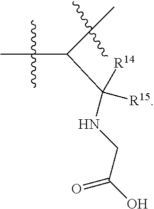

20. (canceled)

21. The conjugate, or a stereoisomer, racemate, tautomer, isotopologue, isotope-labeled compound, nitroxide or pharmaceutically acceptable salt thereof according to claim 14, wherein
L¹ is generated by coupling Tp with a sulfhydryl-reactive group contained in Hal-Het-;
L⁴-L⁵ is: —NR^{z1}—C(R^{z2}R^{z3})-T^{L}-;
wherein:
R¹ is H or C₁₋₄ alkyl;
R^{z2} and R^{z3} are the same or different and are independently selected from H or C₁₋₄ alkyl;
T^{L} has the definition as described in claim 14, and one of T^{L} in L⁴-L⁵ or G is a chemical bond;
further preferably:
when T^{L} in G is a chemical bond, L⁴-L⁵ is: —NR^{z1}—C(R^{z2}R^{z3})—O—(CH₂)ₘ—C(R^{z4}R^{z5})—CO—;
wherein:
R¹ is H or C₁₋₄ alkyl;
m is an integer of 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
particularly, L¹-L² is generated by coupling Tp with L^{1′}-L² of the following structure: 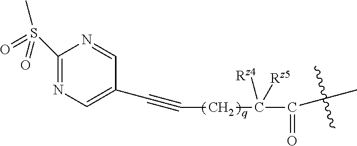
q is an integer selected from 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
further preferably, R^{z4} and R^{z5} together forms a C₃₋₆ cycloalkyl;
L¹-L² is most preferably generated by coupling Tp with

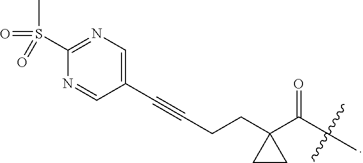 [SMILES: *C(=O)C1(CCC#Cc2cnc(S(C)(=O)=O)nc2)CC1]

22. (canceled)

23. The conjugate, or a stereoisomer, racemate, tautomer, isotopologue, isotope-labeled compound, nitroxide or pharmaceutically acceptable salt thereof according to claim 1,
Tp is a targeting moiety selected from the group consisting of small molecule ligands, proteins, polypeptides, non-protein agents (e.g., sugar, RNA or DNA);
preferably, the target of Tp is selected from the group consisting of epidermal growth factor, Trop-2, CD37, HER2, CD70, EGFRvIII, Mesothelin, Folate receptor1, Mucin 1, CD138, CD20, CD19, CD30, SLTRK6, Nectin 4, Tissue factor, Mucin16, Endothelin receptor, STEAP1, SLC39A6, Guanylylcyclase C, PSMA, CCD79b, CD22, Sodium phosphate cotransporter 2B, GPNMB, Trophoblast glycoprotein, AGS-16, EGFR, CD33, CD66e, CD74, CD56, PD-L1, TACSTD2, DR5, E16, 0772P, MPF, Napi3b, Sema 5b, PSCA hlg, ETBR, MSG783, STEAP2, TrpM4, CRIPTO, CD21, CD79b, FcRH2, NCA, MDP, IL20Rα, Brevican, E phB2R, ASLG659, PSCA, GEDA, BAFF-R, CD79a, CXCR5, HLA-DOB, P2X5, CD72, LY64, FcRH1, IRTA2, TENB2, integrin α5β6, α4β7, FGF2, FGFR2, HER3, CA6, DLL3, DLL4, P-cadherin, EpCAM, pCAD, CD223, LYPD3, LY6E, EFNA4, ROR1, SLITRK6, 5T4, ENPP3, Claudin18.2, BMPR1B, Tyro7, c-Met, ApoE, CD11e, CD40, CD45(PTPRC), CD49D(ITGA4), CD80, CSF1R, CTSD, GZMB, Ly86, MS4A7, PIK3AP1, PIK3CD, CCR5, IFNG, IL10RA1, IL-6, ACTA2, COL7A1, LOX, LRRC15, MCPT8, MMP10, NOG, SERPINE1, STAT1, TGFBR1, CTSS, PGF, VEGFA, C1QA, C1QB, ANGPTL4, EGLN, EGLN3, BNIP3, AIF1, CCL5, CXCL10, CXCL11, IFI6, PLOD2, KISS1R, STC2, DDIT4, PFKFB3, PGK1, PDK1, AKR1C1, AKR1C₂, CADM1, CDH11, COL6A3, CTGF, HMOX1, KRT33A, LUM, WNT5A, IGFBP3, MMP14, CDCP1, PDGFRA, TCF4, TGF, TGFB1, TGFB2, CD11b, ADGRE1, EMR2, TNFRSF21, UPK1B, TNFSF9, MMP16, MFI2, IGF-1R, RNF43, NaPi2b and BCMA;
preferably, Tp is a small molecule ligand, such as a folic acid derivative, a glutamate urea derivative, a somatostatin derivative, an arylsulfonamide derivative (e.g., a carbonic anhydrase IX inhibitor), an ICG dye, a cyanine dye or a derivative thereof;
preferably, Tp is selected from an antibody or antigen-binding fragment thereof, and the antibody is selected from the group consisting of chimeric antibody, humanized antibody or fully human antibody; preferably monoclonal antibody;
preferably, the antibody or antigen-binding fragment thereof is at least one selected from the following antibodies or antigen-binding fragments: anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-HER2 (ErbB2) antibody, anti-EGFR antibody, anti-B7-H3 antibody, anti-c-Met antibody, anti-HER3 (ErbB3) antibody, anti-HER4 (ErbB4) antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, the antibody can be a bispecific antibody or a multispecific antibody;
further preferably, the antibody or antigen-binding fragment thereof is at least one selected from the following antibodies or antigen-binding fragments: Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR 96, and Glematumamab or Glembatumumab;
for example, Trastuzumab has sequences selected from the following:
light chain
SEQ ID NO: 1
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO: 2
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK
Pertuzumab has sequences selected from the following:
light chain
SEQ ID NO. 3
DIQMTQSPSSLSASVGDRVTITCKASQDVSIGVAWYQQKPGKAPK
LLIYSASYRYTGVPSRFSGSGSGTDFTLTISSLQPEDFATYYCQQ
YYIYPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
heavy chain
SEQ ID NO. 4
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK
Nimotuzumab has sequences selected from the following:
light chain
SEQ ID NO: 5
DIQMTQSPSSLSASVGDRVTITCRSSQNIVHSNGNTYLDWYQQTPG
KAPKLLIYKVSNRFSGVPSRFSGSGSGTDFTFTISSLQPEDIATY
YCFQYSHVPWTFGQGTKLQITREVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
heavy chain
SEQ ID NO: 6
QVQLQQSGAEVKKPGSSVKVSCKASGYTFTNYYIYWVRQAPGQGL
EWIGGINPTSGGSNFNEKFKTRVTITVDESTNTAYMELSSLRSED
TAFYFCARQGLWFDSDGRGFDFWGQGSTVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK
Patritumab has following sequences:
light chain
SEQ ID NO: 7
DIEMTQSPDSLAVSLGERATINCRSSQSVLYSSSNRNYLAWYQQN
PGQPPKLLIYWASTRESGVPDRFSGSGSGTDFTLTISSLQAEDVA
VYYCQQYYSTPRTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
heavy chain
SEQ ID NO: 8
QVQLQQWGAGLLKPSETLSLTCAVYGGSFSGYYWSWIRQPPGKGL
EWIGEINHSGSTNYNPSLKSRVTISVETSKNQFSLKLSSVTAADT
AVYYCARDKWTWYFDLWGRGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKRVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK.

24. The conjugate, or a stereoisomer, racemate, tautomer, isotopologue, isotope-labeled compound, nitroxide or pharmaceutically acceptable salt thereof according to claim 1, wherein the conjugate, or linker or linker-drug thereof is selected from one of the following:
wherein, u, v, w are independently selected from an integer of 0 to 10 (e.g., 0, 1, 2, 3, 4, 5, 6, 7, 8, 9 or 10), G has the definition as described in claim 1, LG has the definition of Tp of claim 1; R²⁰, R^{20′} are the same or different and are independently selected from the group consisting of H, C₁₋₄ alkyl, C₃₋₆ cycloalkyl, C₃₋₆ cycloalkyl-C₁₋₄ alkyl, or R²⁰, R^{20′} and the carbon atom to which they are attached together form a C₃₋₆ cycloalkyl;
for example, the C₁₋₄ alkyl can be independently selected from the group consisting of methyl, ethyl, propyl, isopropyl, butyl, isobutyl or tert-butyl; the C₃₋₆ cycloalkyl can be independently selected from the group consisting of cyclopropyl, cyclobutyl, cyclopentyl or cyclohexyl;
for example, both of R²⁰ and R^{20′} are H, or one of them is not H, or both of them are not H:

| No. | Conjugate, or linker or linker-drug thereof |
|---|---|
| 1 | 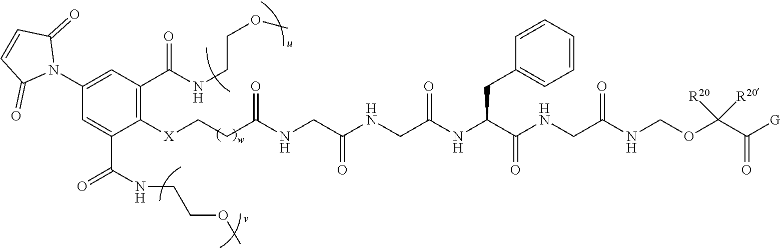 |
| 2 | 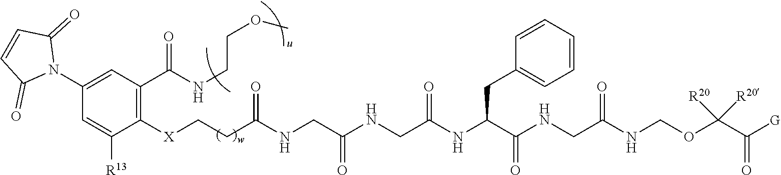 |
| 3 | 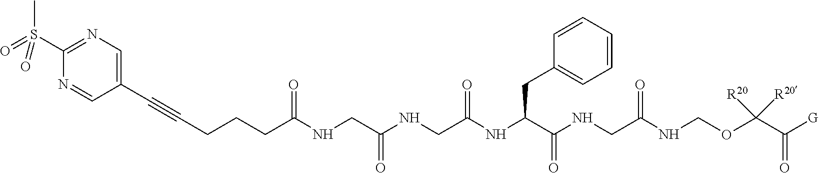 |
| 4 | 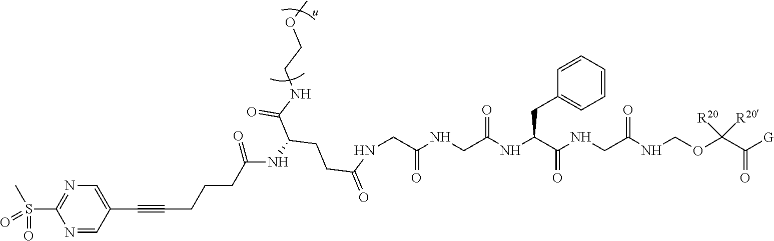 |
| 5 | 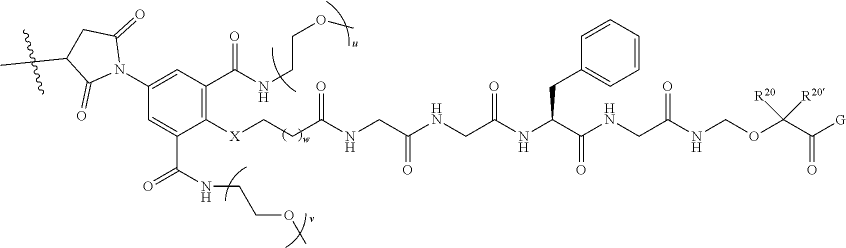 |
| 6 | 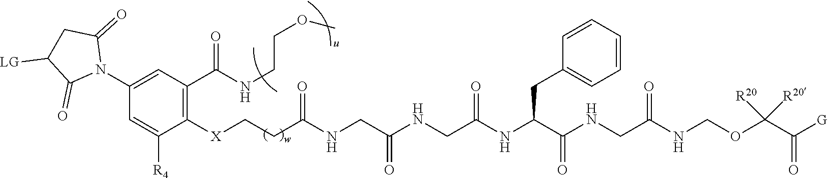 |
| 7 | 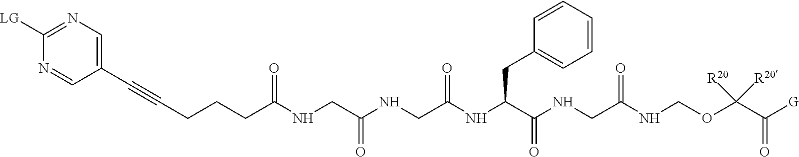 |
| 8 | 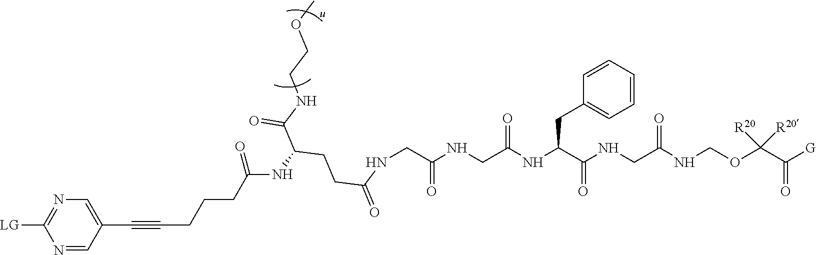 |
| 9 | 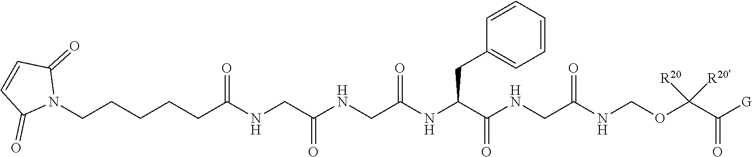 |
| 10 | 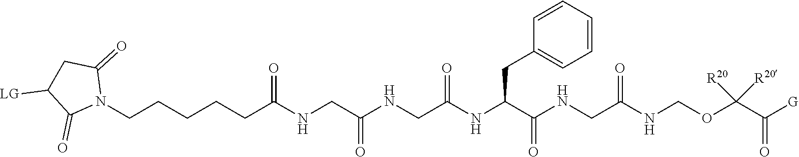 |
| 11 | 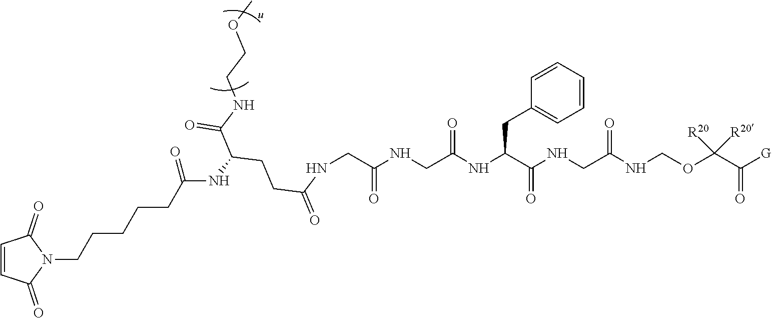 |
| 12 | 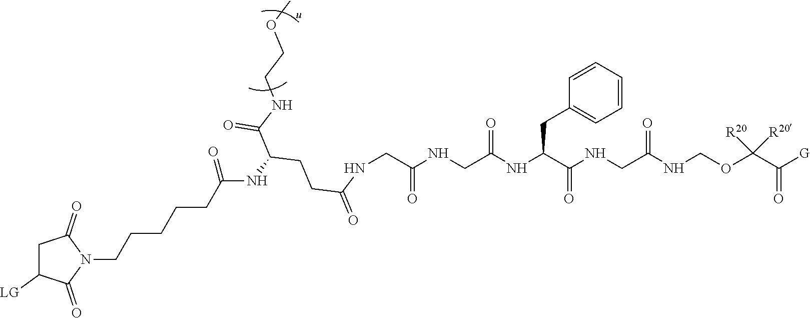 |
| 13 | 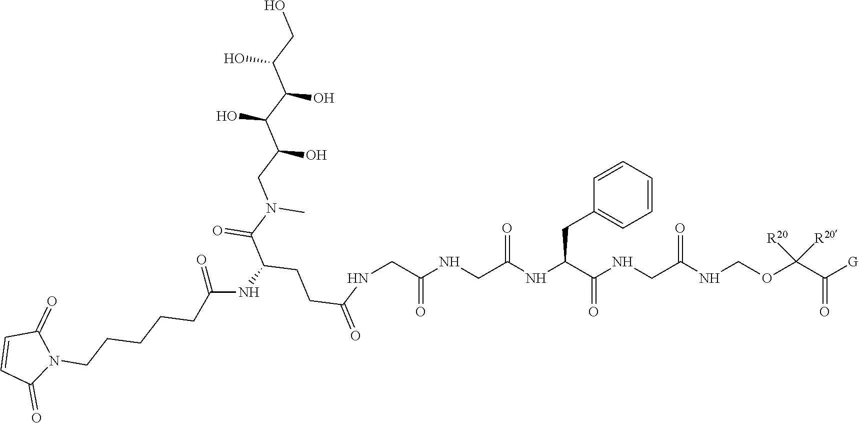 |
| 14 | 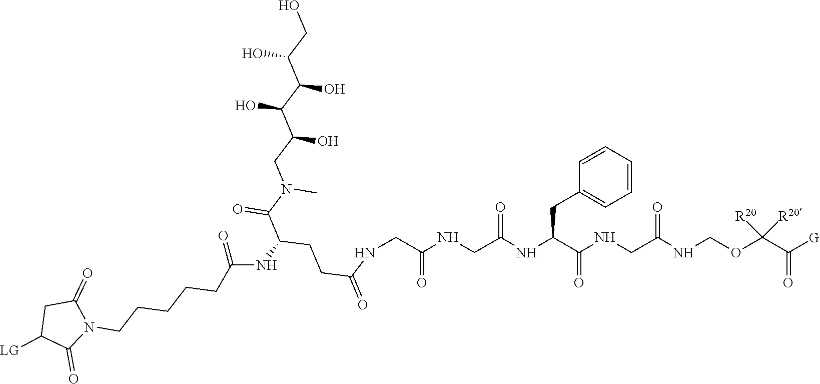 |
| 15 | 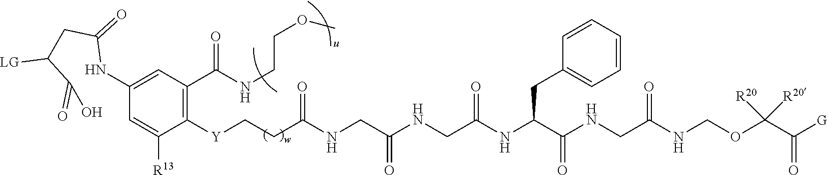 |
| 16 | 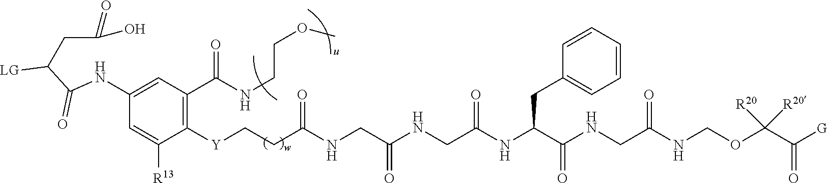 |
| 17 | 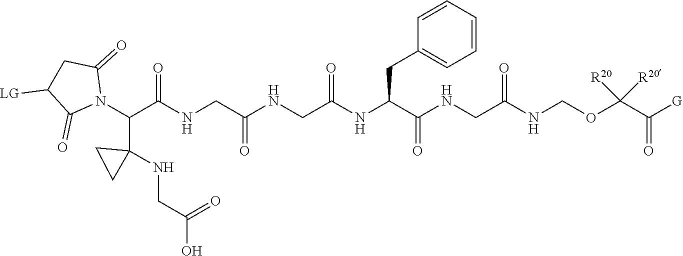 |
| 18 | 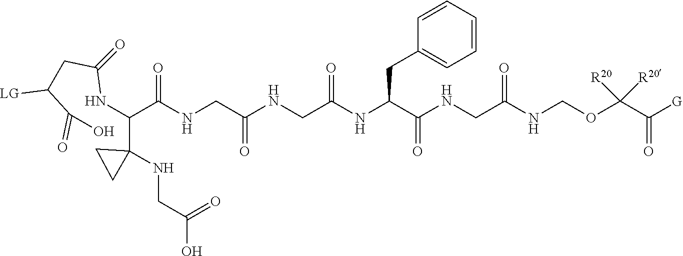 |
| 19 | 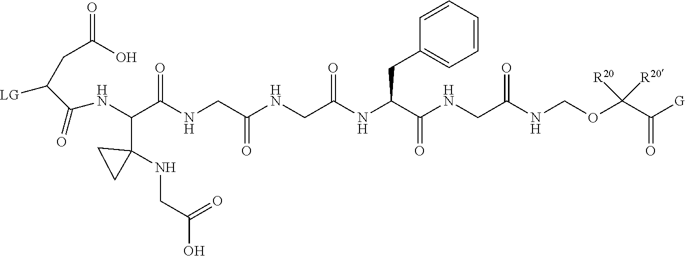 |
| 20 | 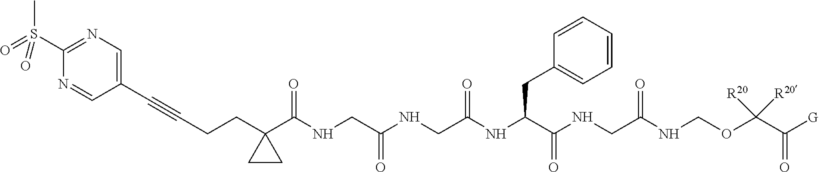 |
| 21 | 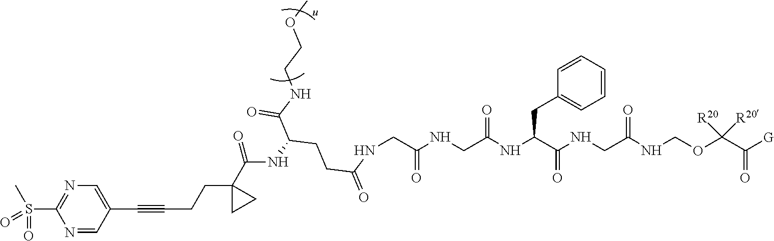 |
| 22 | 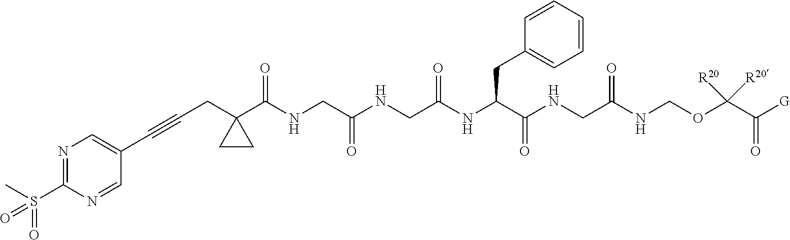 |
| 23 | 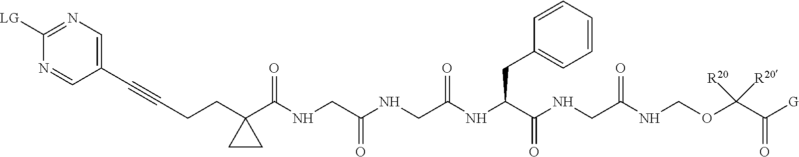 |
| 24 | 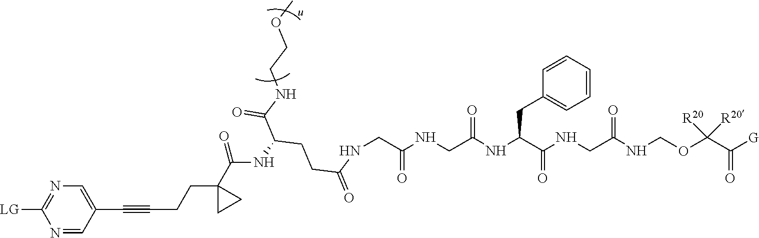 |
| 25 | 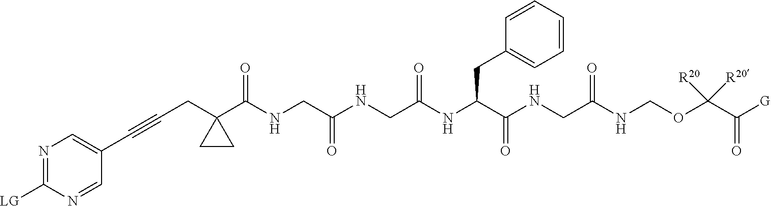 |
| 26 | 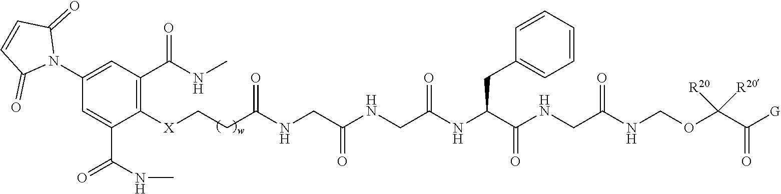 |
| 27 | 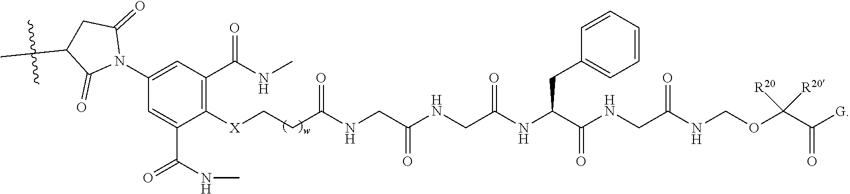 |

25. The conjugate, or a stereoisomer, racemate, tautomer, isotopologue, isotope-labeled compound, nitroxide or pharmaceutically acceptable salt thereof according to claim 1, wherein the conjugate has a structure as shown in any of the following formulas: 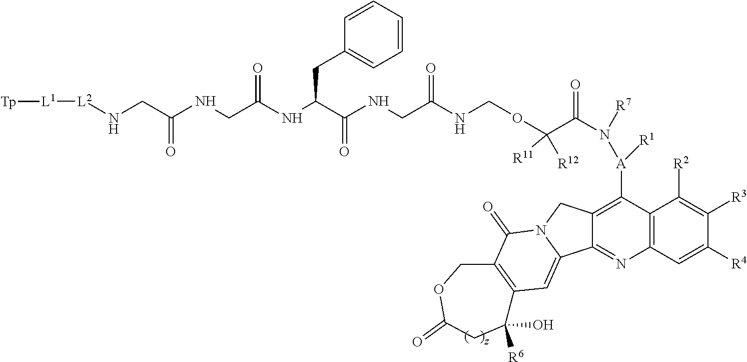
wherein, z, A, R¹, R², R³, R⁴, R⁶, R⁷, R¹¹, R¹², L, L², and Tp have the definitions as described in claim 1; 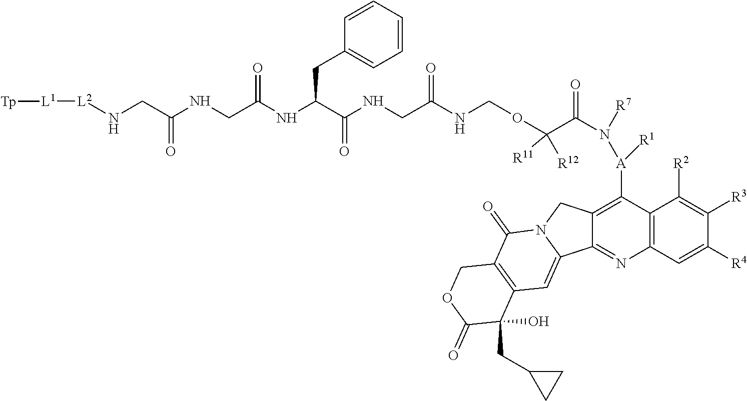
wherein, A, R¹, R², R³, R⁴, R⁷, R¹, R¹², L¹, L², and Tp have the definitions as described in claim 1; 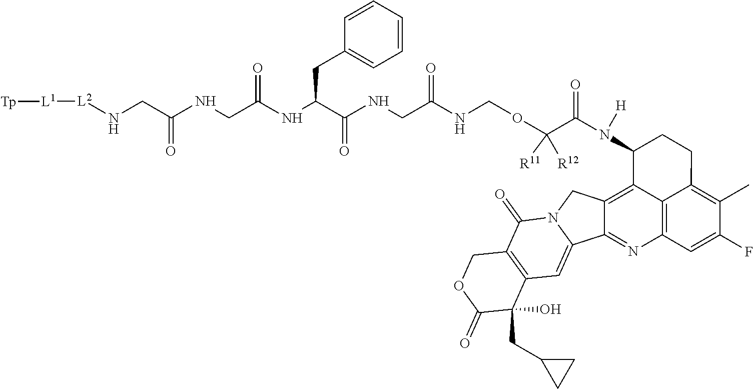
wherein, R¹¹, R¹², L¹, L², and Tp have the definitions as described in claim 1; 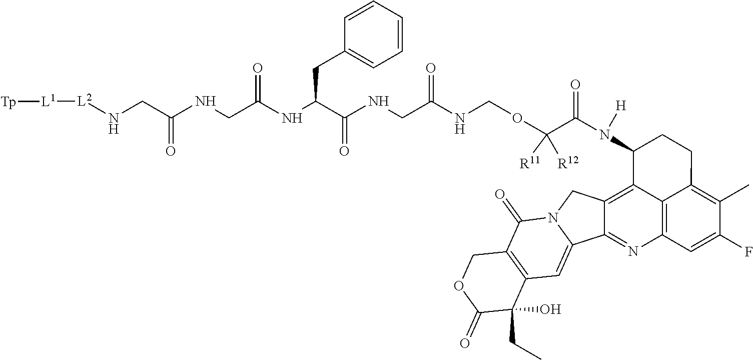
wherein, R¹¹, R¹², L¹, L², and Tp have the definitions as described in claim 1; 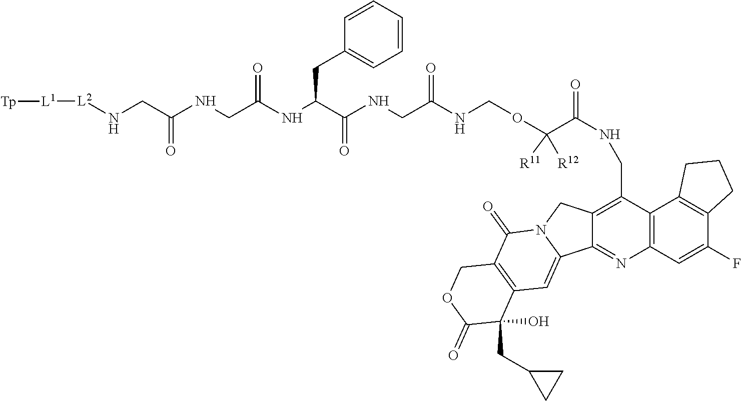
wherein, R¹¹ and R¹² independently have the definitions as described in claim 1; for example, R¹¹ and R¹² are the same or different and are independently selected from the group consisting of H, C₁₋₄ alkyl, C₃₋₆ cycloalkyl, C₃₋₆ cycloalkyl-C₁₋₄ alkyl, or R¹¹, R¹² and the carbon atom to which they are connected together form a C₃₋₆ cycloalkyl;
for example, the C₁₋₄ alkyl can be independently selected from the group consisting of methyl, ethyl, propyl, isopropyl, butyl, isobutyl or tert-butyl; the C₃₋₆ cycloalkyl can be independently selected from the group consisting of cyclopropyl, cyclobutyl, cyclopentyl or cyclohexyl;
for example, both of R¹¹ and R¹² are H, or one is not H, or both are not H;
L¹, L², and Tp independently have the definitions as described in claim 1.

26-27. (canceled)

28. The conjugate, or a stereoisomer, racemate, tautomer, isotopologue, isotope-labeled compound, nitroxide or pharmaceutically acceptable salt thereof according to claim 1, wherein the conjugate is one selected from the followings: 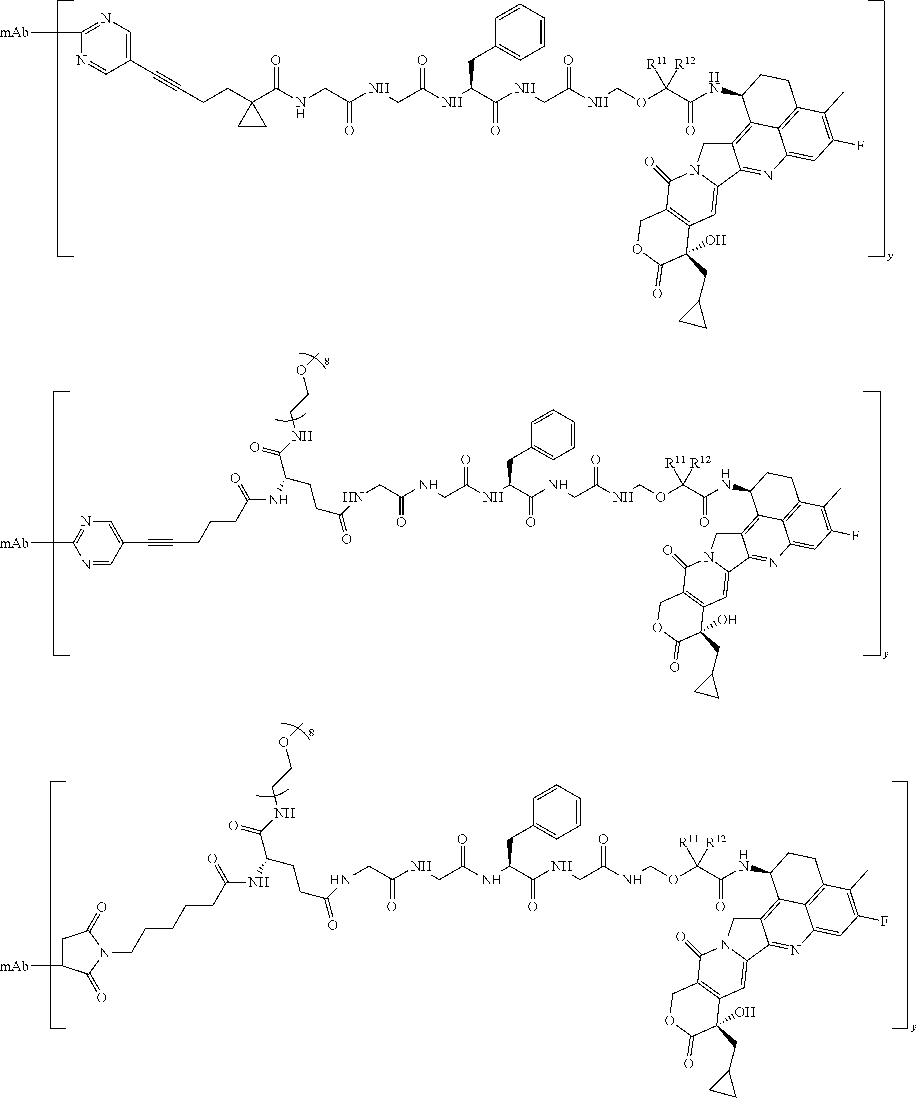 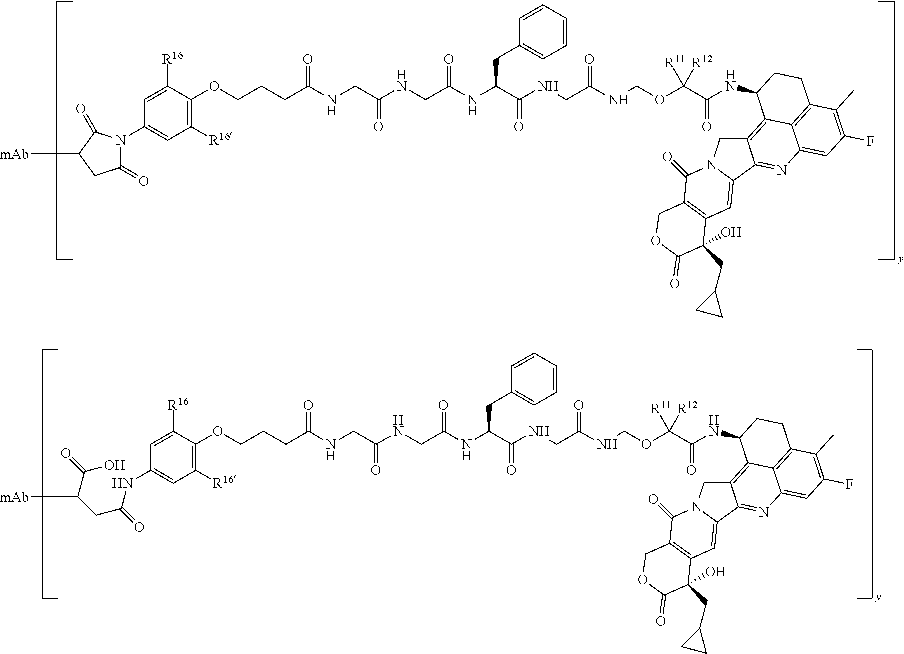 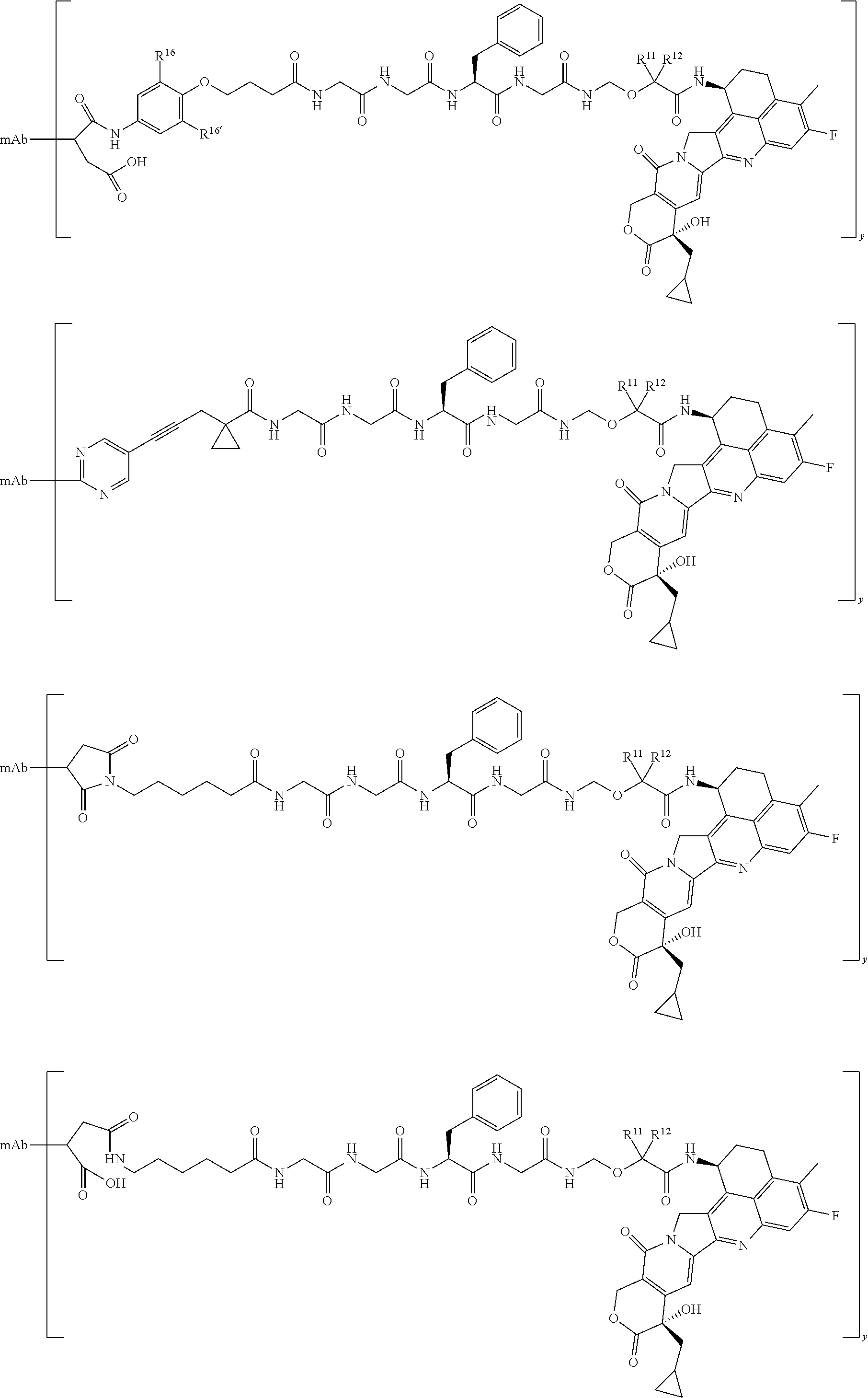 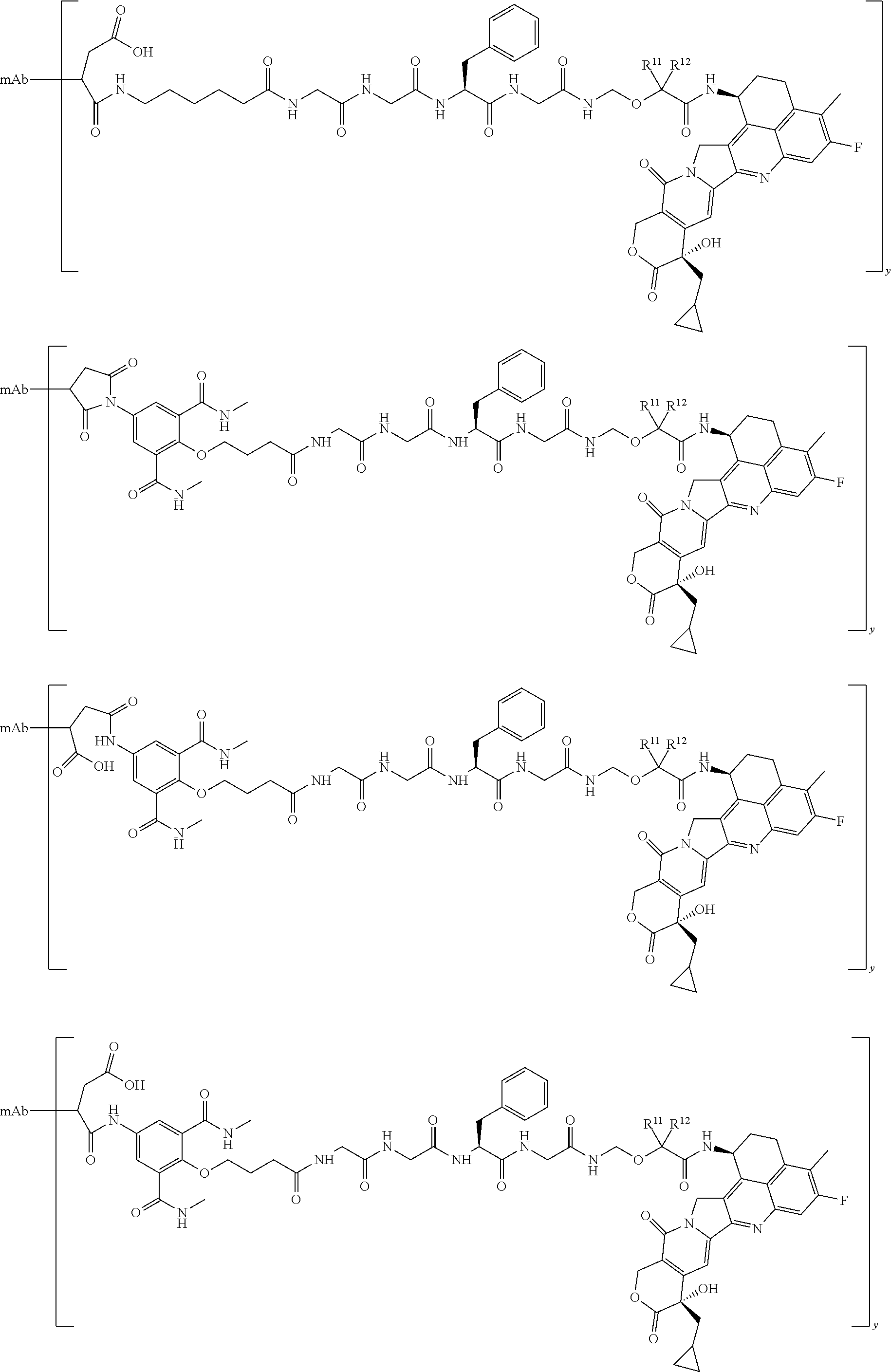 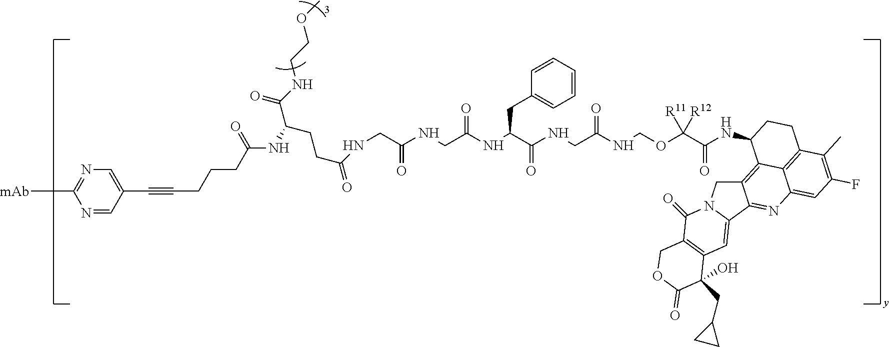
wherein, R¹¹ and R¹² are the same or different and independently have the definitions as described in claim 1; for example, R¹¹ and R¹² are the same or different and are independently selected from the group consisting of H, C₁₋₄ alkyl, C₃₋₆ cycloalkyl, C₃₋₆ cycloalkyl-C₁₋₄ alkyl, or R¹¹ and R¹², together with the carbon atom to which they are attached, form a C₃₋₆ cycloalkyl;
for example, the C₁₋₄ alkyl group can be independently selected from the group consisting of methyl, ethyl, propyl, isopropyl, butyl, isobutyl or tert-butyl; the C₃₋₆ cycloalkyl group can be independently selected from the group consisting of cyclopropyl, cyclobutyl, cyclopentyl or cyclohexyl;
for example, both of R¹¹ and R¹² are H, or one of them is not H, or both of them are not H;
R¹⁶ and R^{16′} are the same or different, and at least one of them is selected from a hydrophilic group, and the other is selected from the following substituents: hydrogen, halogen, cyano, amino, nitro, and the following groups which are unsubstituted or substituted with one, two or more R^{zg}: C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy, aminocarbonyl;
the hydrophilic group is selected from a polyethylene glycol group, a C₁₋₁₀ alkyl substituted with 1 to 10 hydroxyl groups, or a sugar ring-containing group, or C₁₋₆ alkyl-NHCO— (e.g., C₁₋₄ alkyl-NHCO—), preferably a polyethylene glycol group, more preferably —C(═O)—NH—(CH₂CH₂O)ₚ—C₁₋₁₀ alkyl or —NH—(CH₂CH₂O)ₚ—C₁₋₁₀ alkyl, or preferably a C₁₋₁₀ alkyl substituted with 1 to 10 hydroxyl groups, more preferably 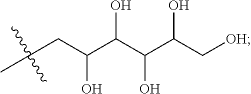 [SMILES: *CC(O)C(O)C(O)C(O)CO]
each p is the same or different, and is independently selected from an integer of 0 to 10, preferably 1, 2, 3, 4, 5, 6, 7 or 8;
R^{zg} is selected from the group consisting of halogen, hydroxyl, amino, cyano, nitro, C₁₋₁₀ alkyl, C₁₋₁₀ alkyloxy, C₃₋₁₀ cycloalkyl, C₃₋₁₀ cycloalkyl-C₁₋₁₀ alkyl, C₃₋₁₀ cycloalkyloxy;
mAb represents a monoclonal antibody;
y represents the average number of small molecule drugs attached to each monoclonal antibody (DAR), which can be selected from an integer or decimal, for example, an integer or decimal selected from 1 to 50, an integer or decimal selected from 1 to 20, or an integer or decimal selected from 1 to 10.

29-32. (canceled)

33. The conjugate, or a stereoisomer, racemate, tautomer, isotopologue, isotope-labeled compound, nitroxide or pharmaceutically acceptable salt thereof according to claim 1, wherein the conjugate is one selected from the following:
y represents the average number of small molecule drugs attached to each monoclonal antibody (DAR), which can be selected from an integer or decimal, such as an integer or decimal selected from 1 to 50, an integer or decimal selected from 1 to 20, or an integer or decimal selected from 1 to 10; 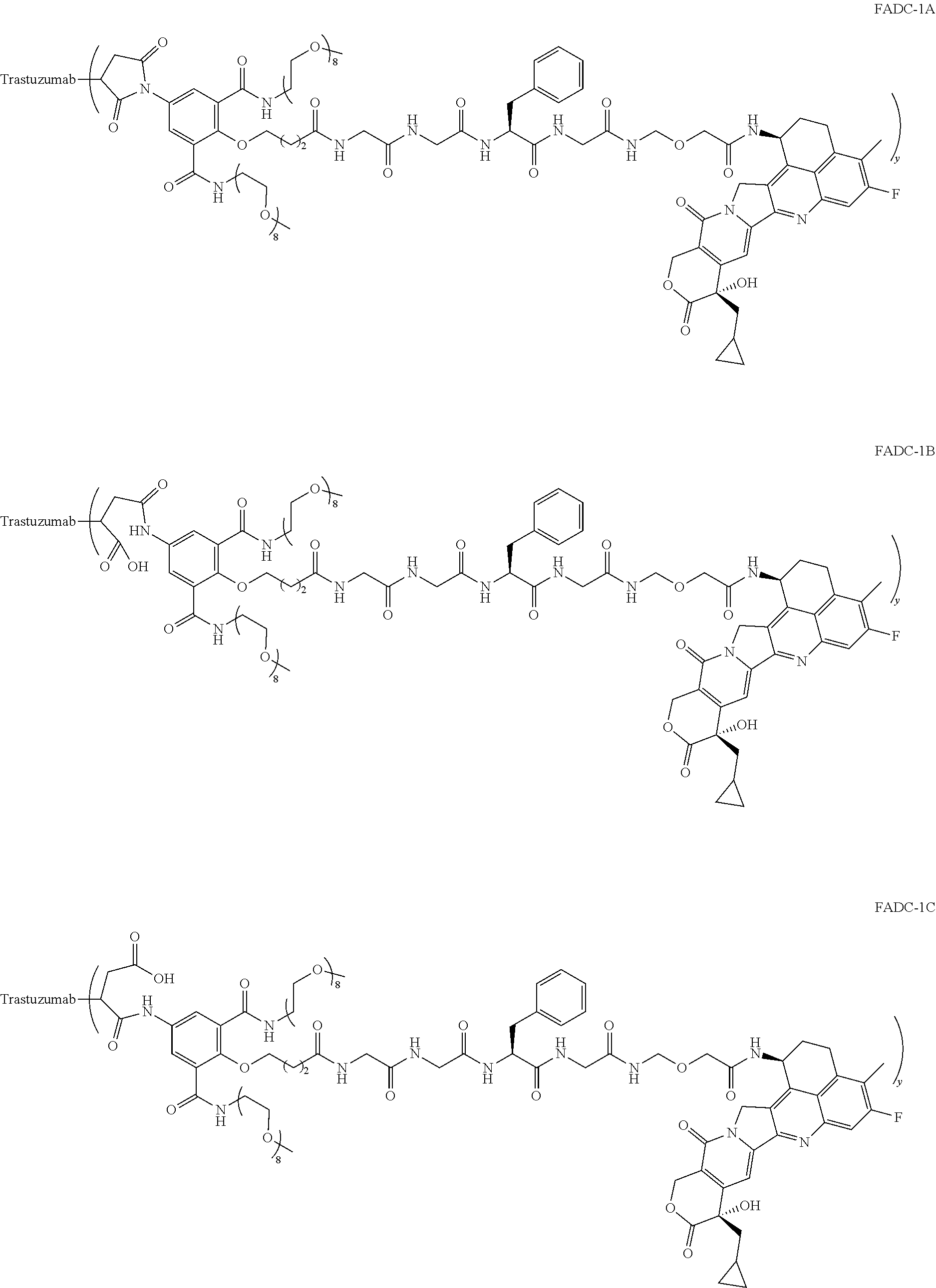 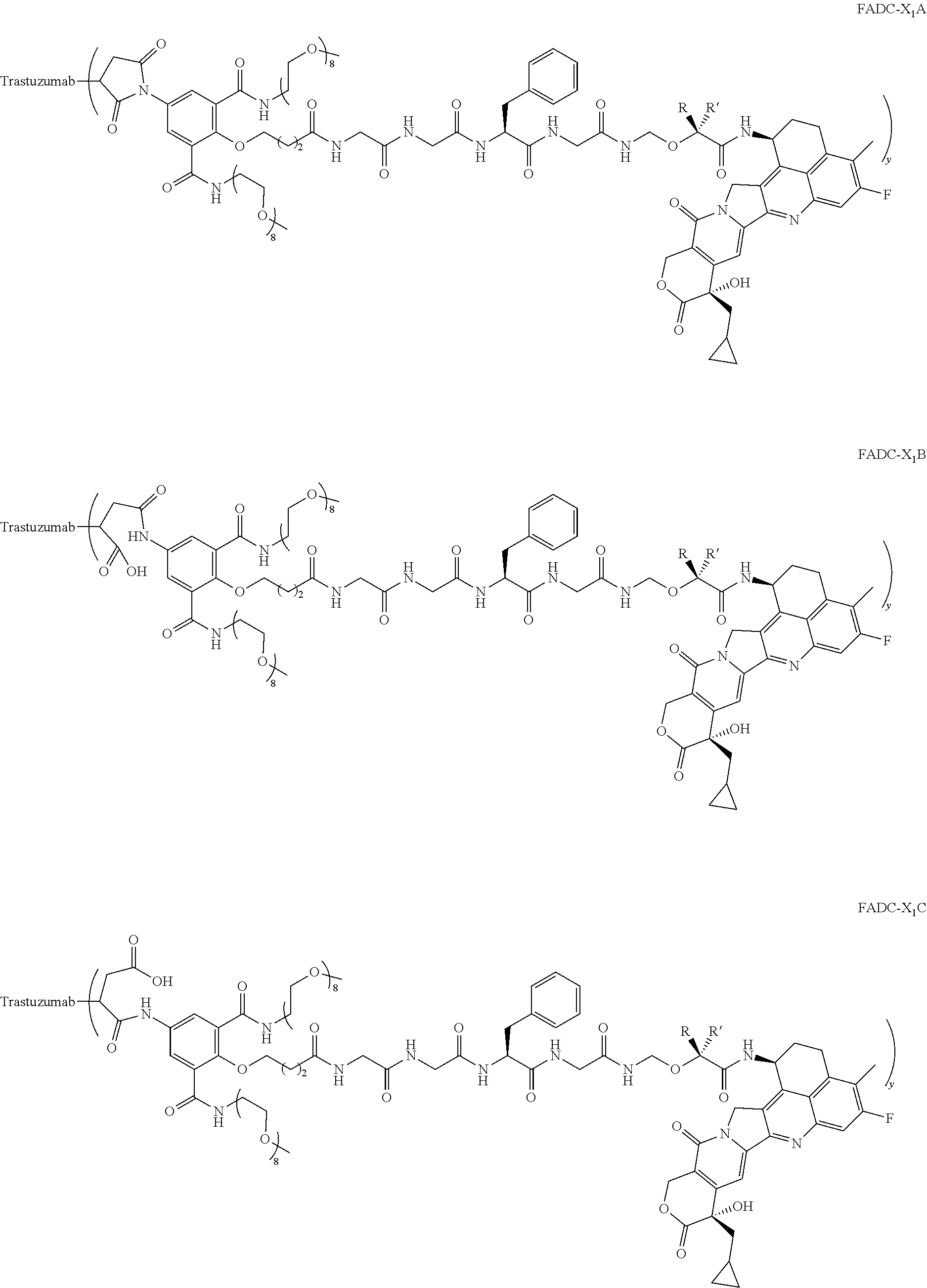
wherein, R is selected from —CH₃, R′ is selected from —H;
R is selected from —H, R′ is selected from —CH₃;
R is selected from 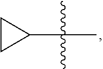 [SMILES: *C1CC1]
R′ is selected from from —H; or
R is selected from —H, R′ is selected from 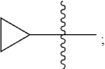 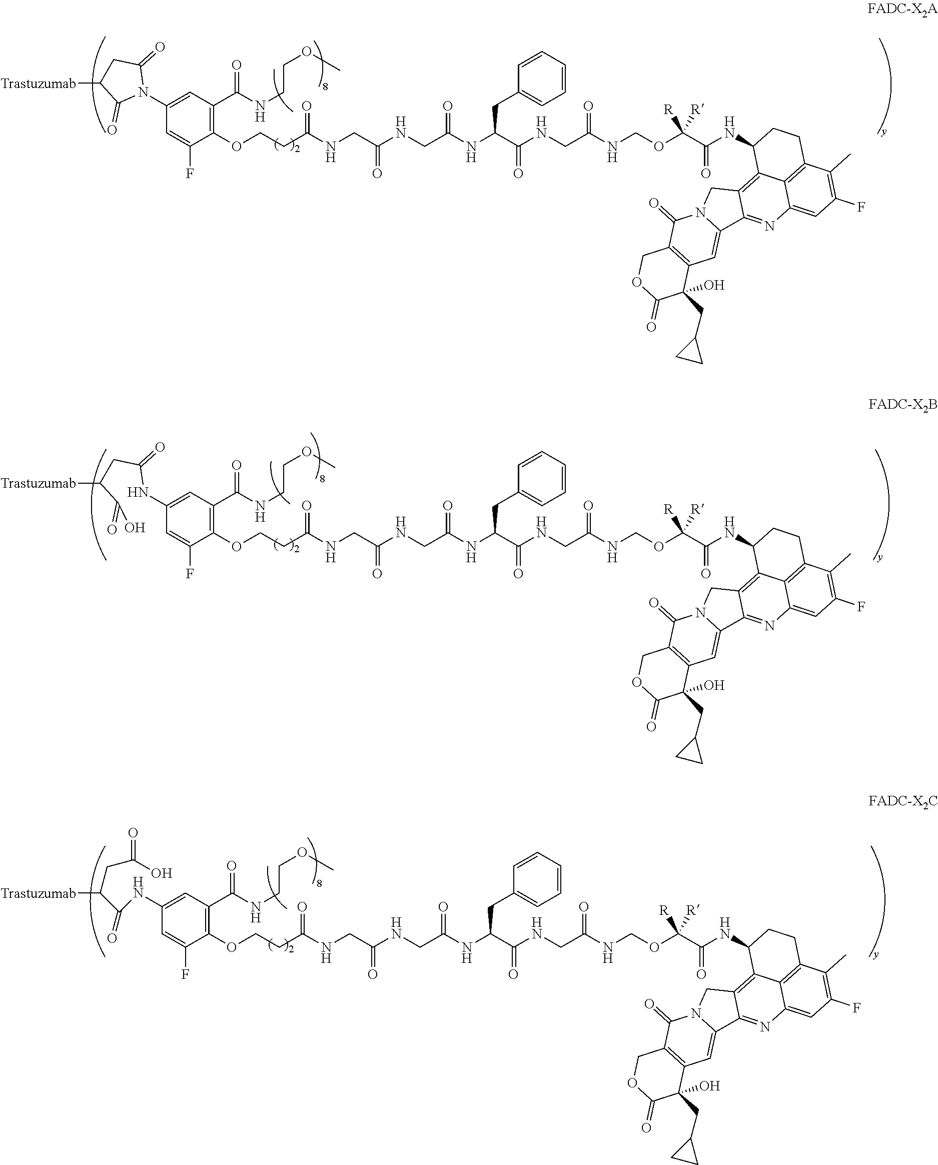
wherein, R is selected from —H, R′ is selected from —H;
R is selected from —CH₃, R′ is selected from —H;
R is selected from —H, R′ is selected from —CH₃;
R is selected from 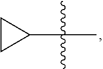 [SMILES: *C1CC1]
R′ is selected from —H; or
R is selected from —H, R′ is selected from 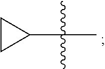 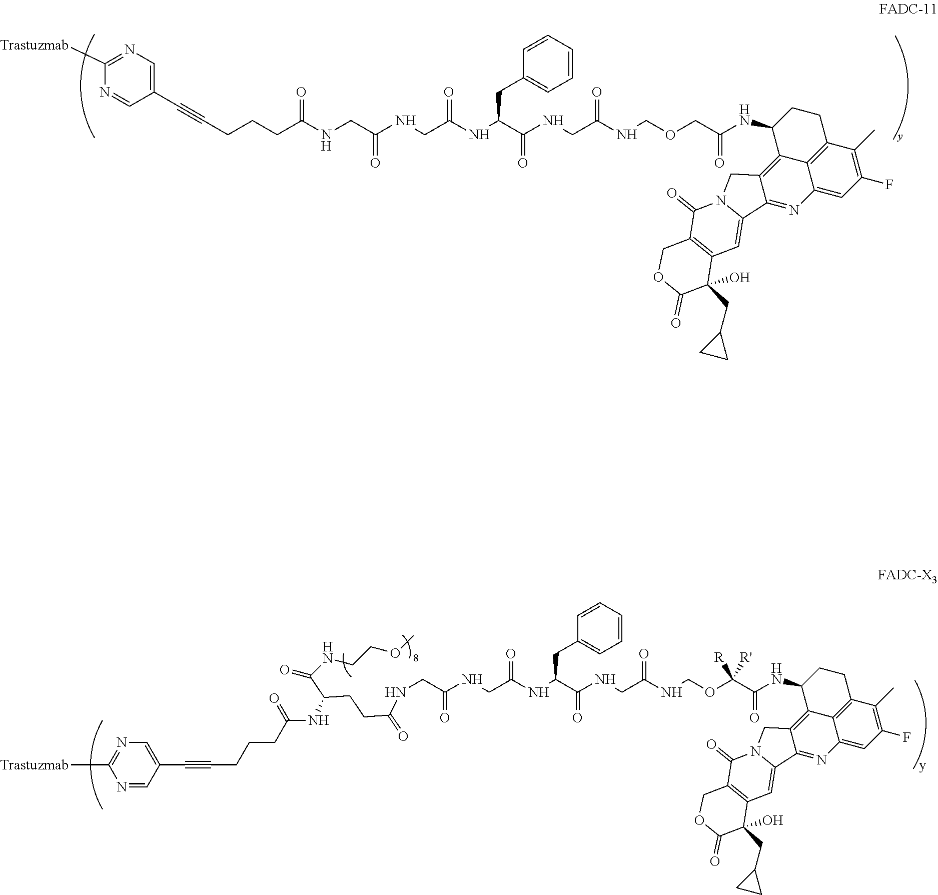
wherein, R is selected from —H, R′ is selected from —H;
R is selected from —CH₃, R′ is selected from —H;
R is selected from —H, R′ is selected from —CH₃;
R is selected from 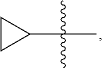 [SMILES: *C1CC1]
R′ is selected from —H; or
R is selected from —H, R′ is selected from 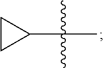 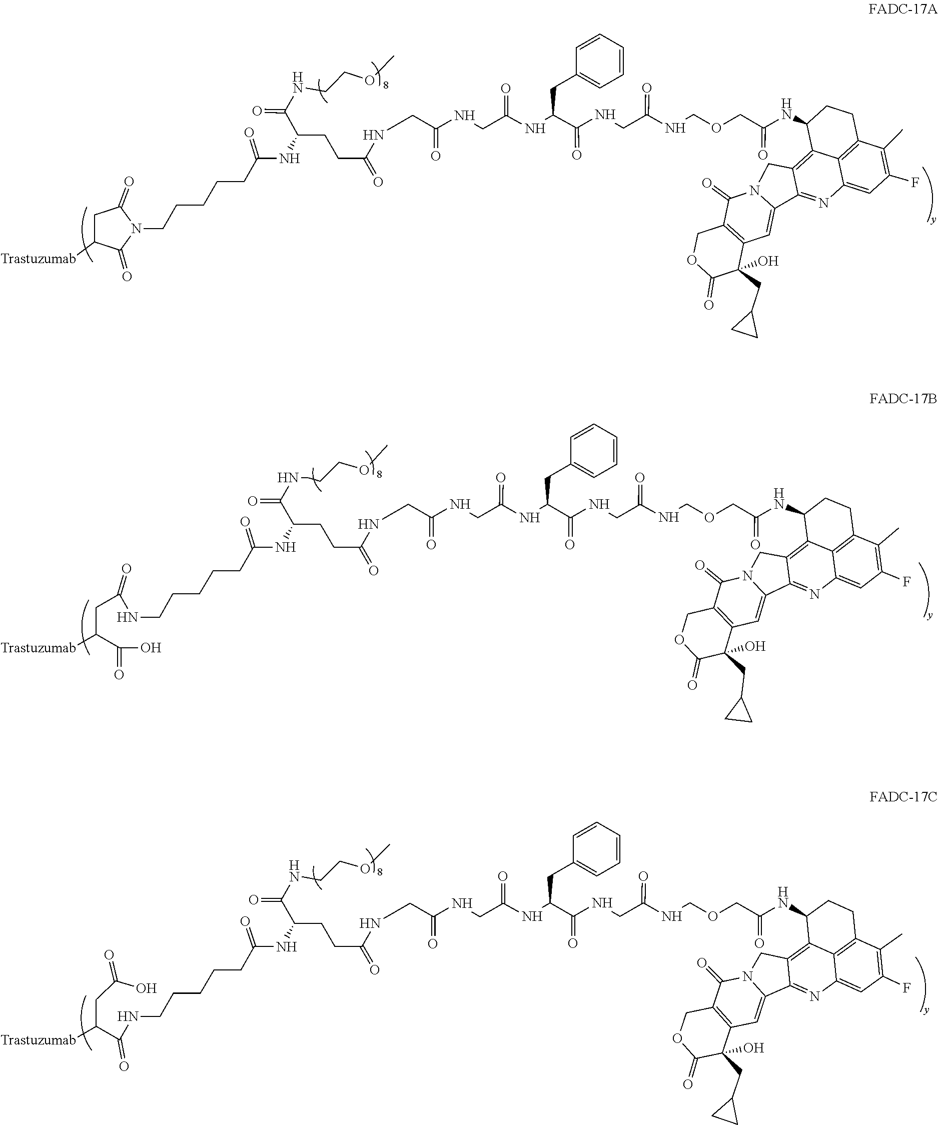 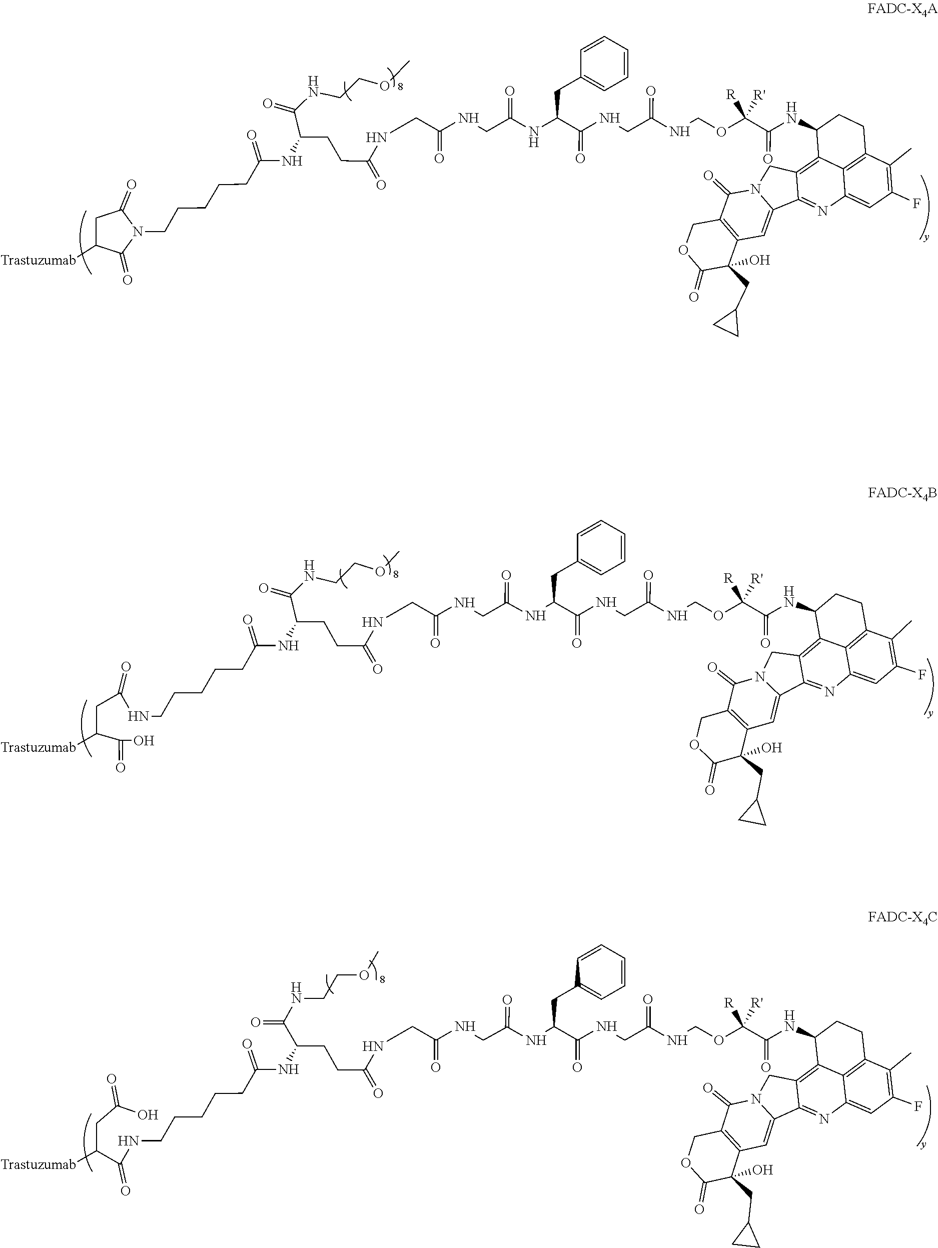
wherein, R is selected from —CH₃, R′ is selected from —H;
R is selected from —H, R′ is selected from —CH₃;
R is selected from 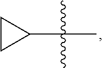 [SMILES: *C1CC1]
R is selected from —H; or
R is selected from —H, R′ is selected from 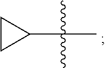 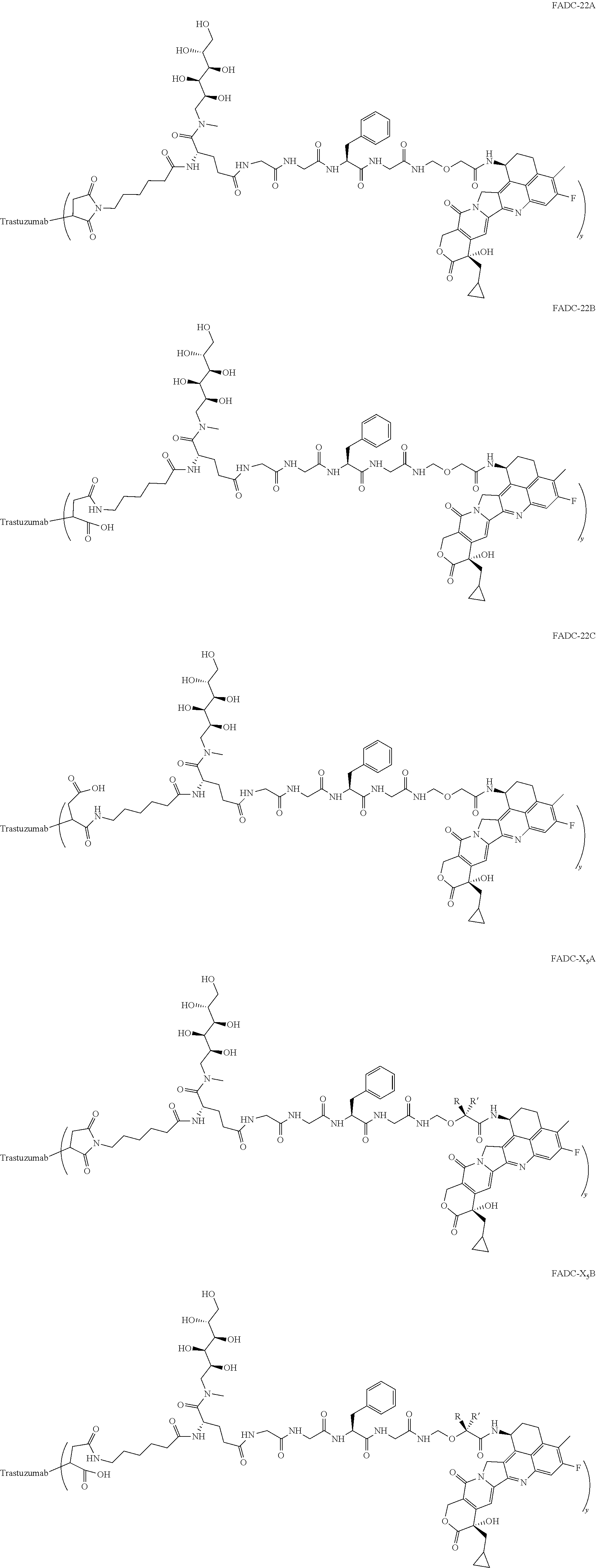 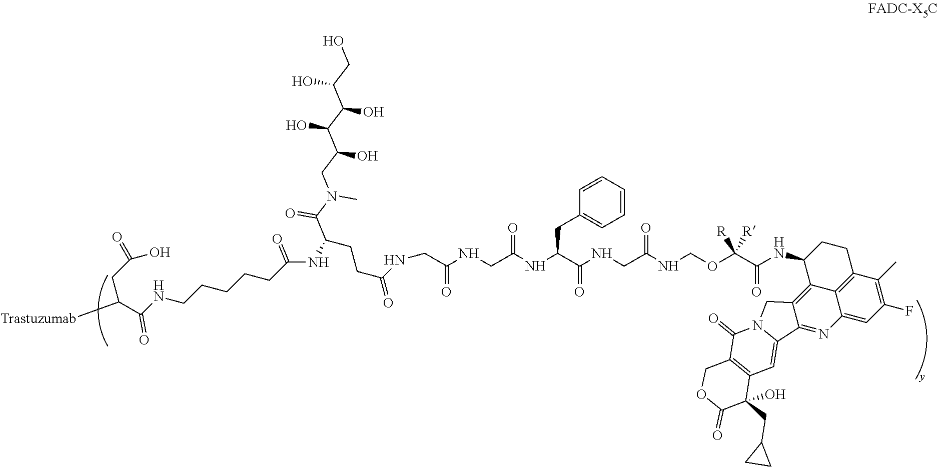
wherein, R is selected from —CH₃, R′ is selected from —H;
R is selected from —H, R′ is selected from —CH₃;
R is selected from 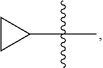 [SMILES: *C1CC1]
R′ is selected from —H; or
R is selected from —H, R′ is selected from 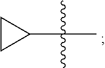 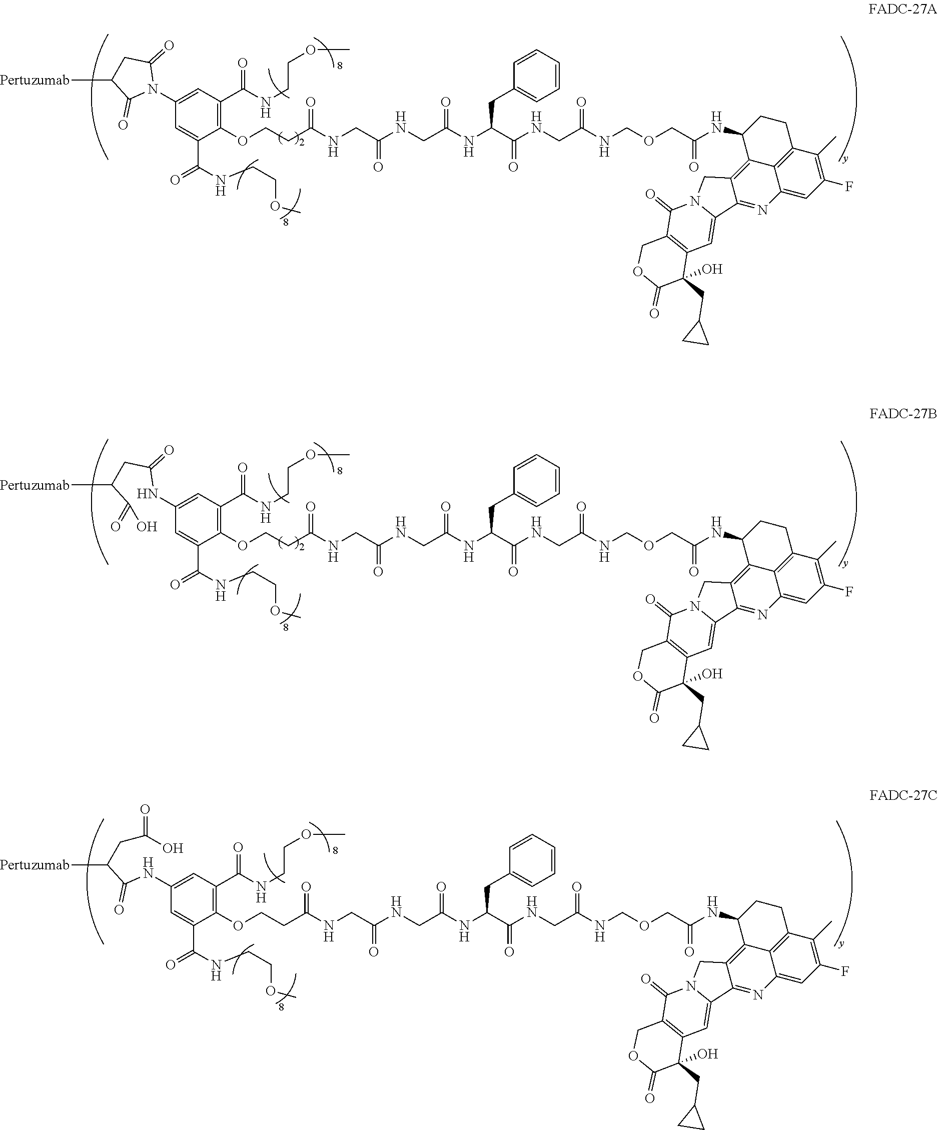 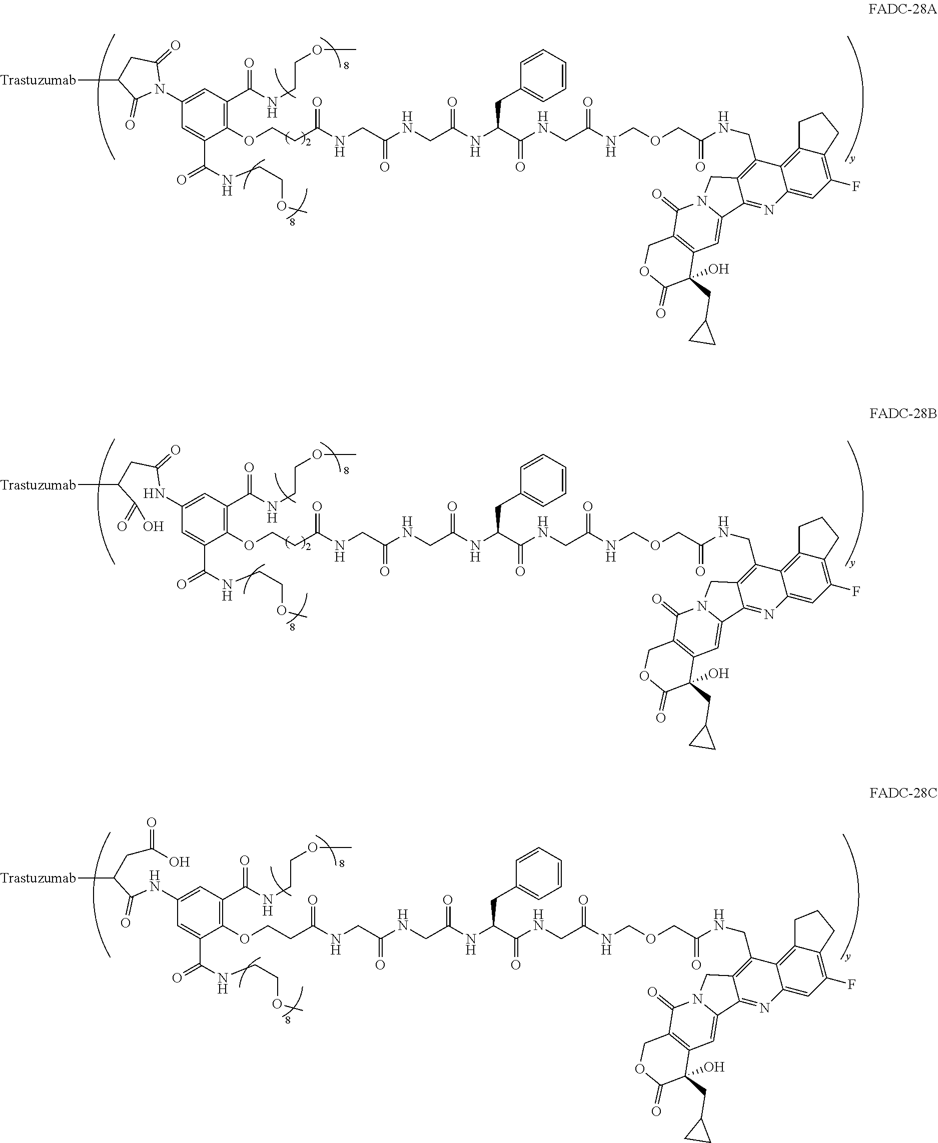 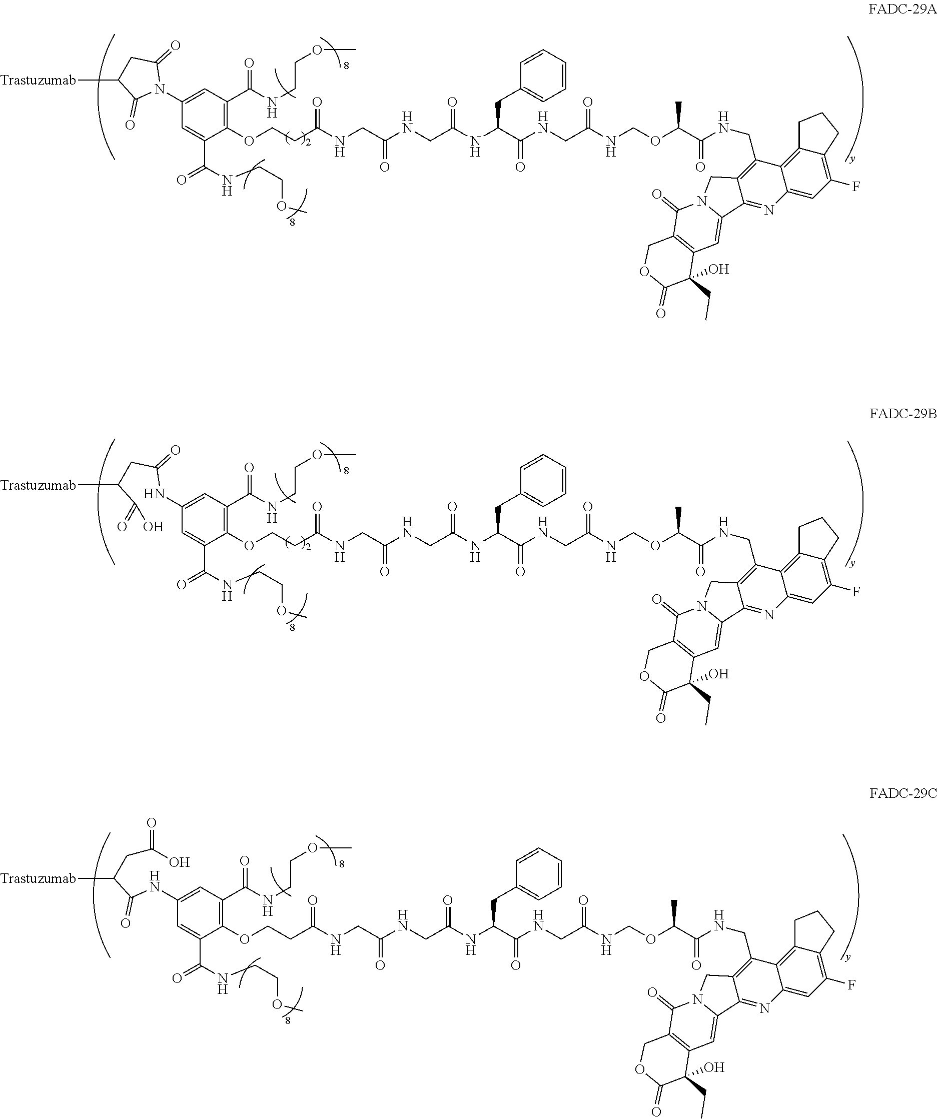 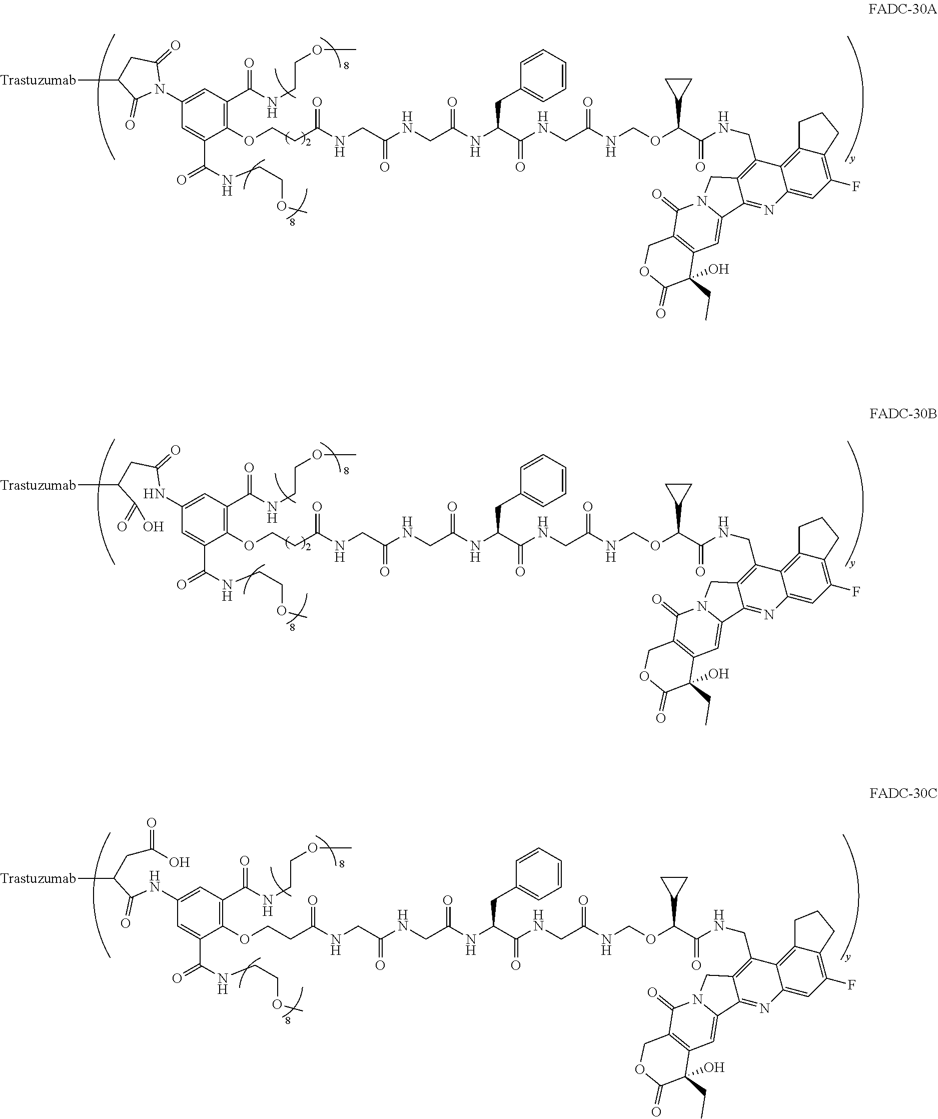 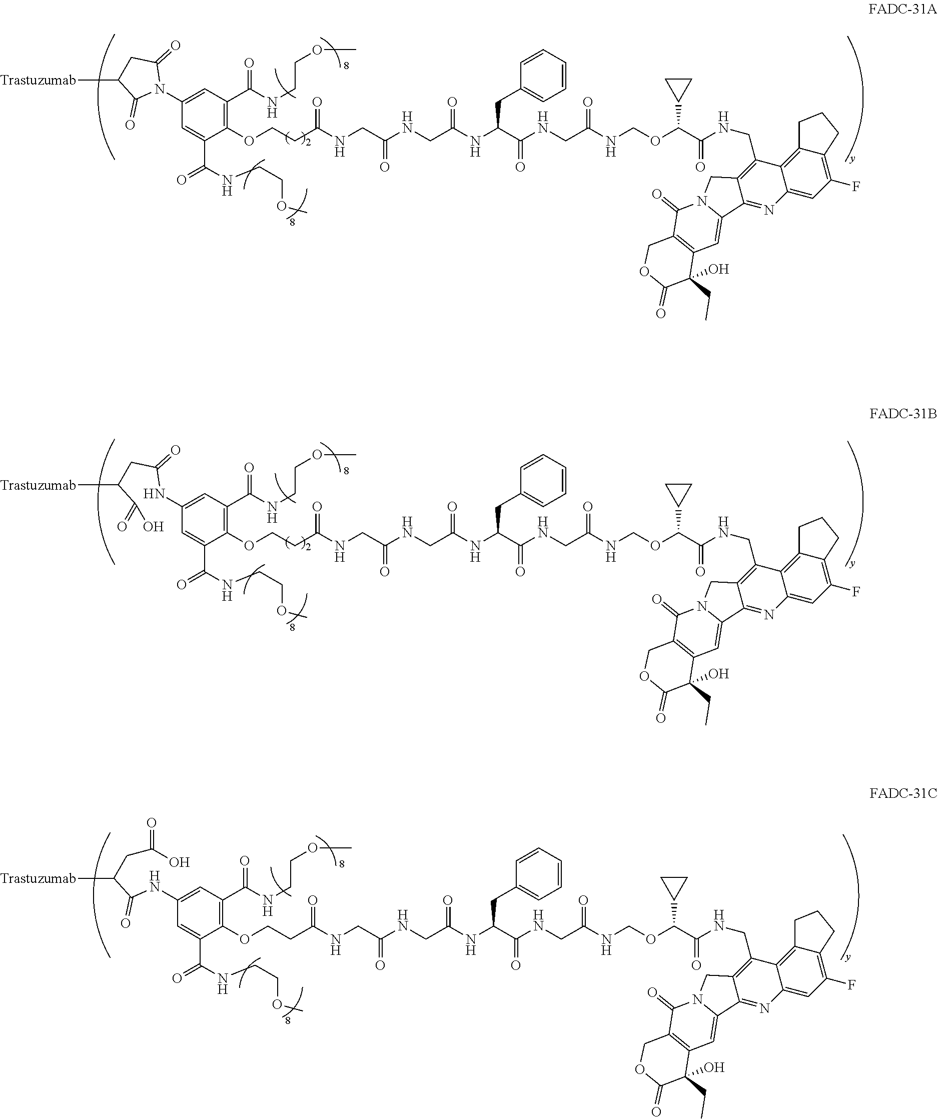 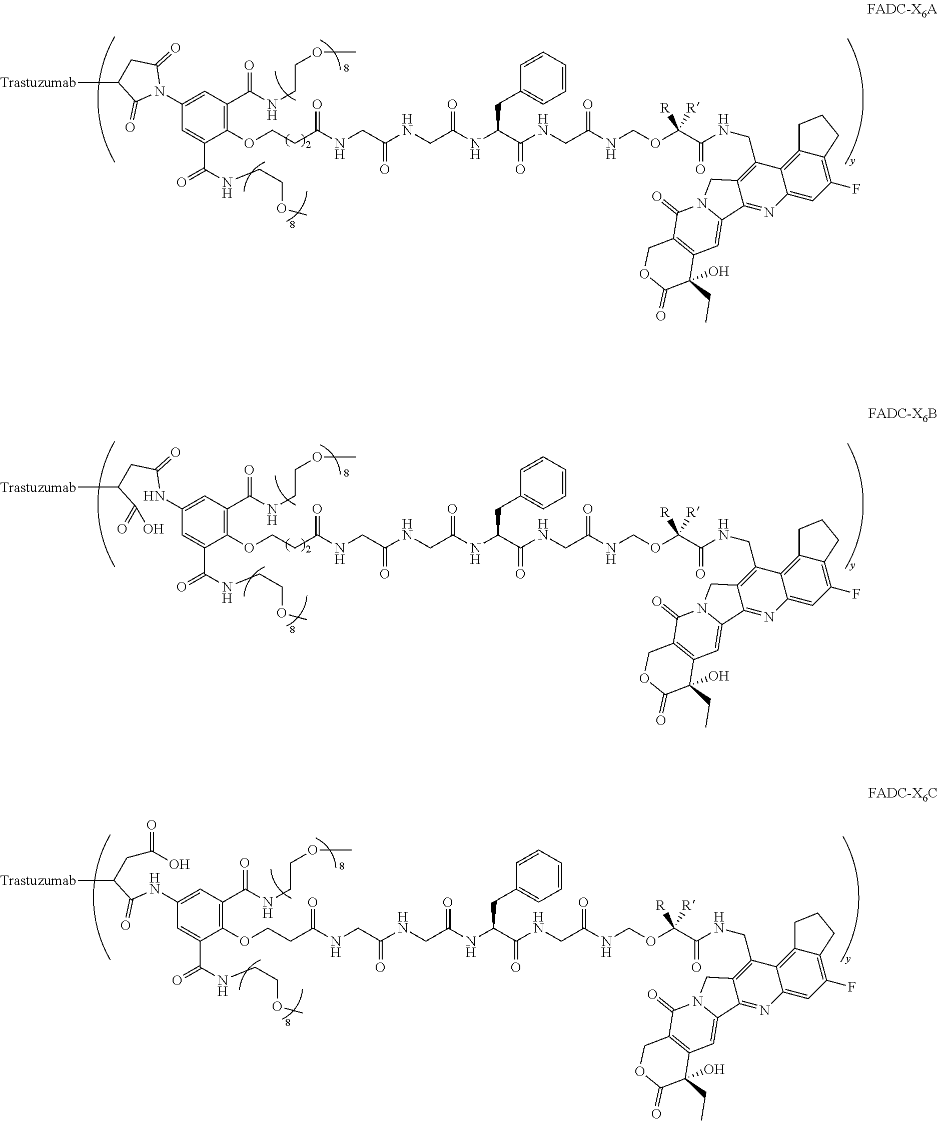
wherein, R is selected from —H, R′ is selected from —H;
R is selected from —CH₃, R′ is selected from —H;
R is selected from 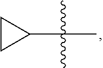 [SMILES: *C1CC1]
R′ is selected from —H; or
R is selected from —H, R′ is selected from 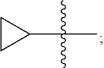 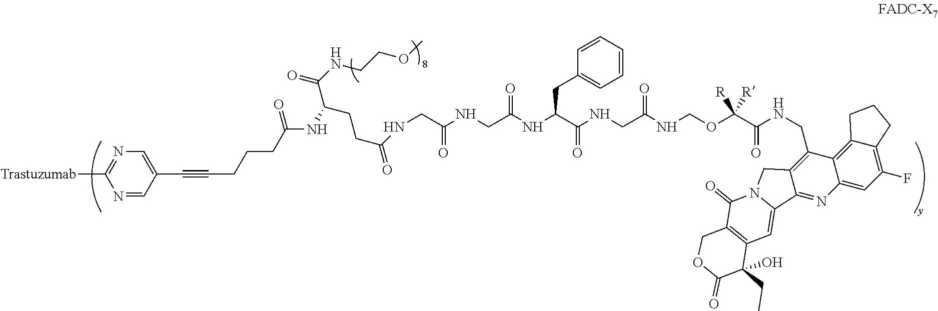
wherein, R is selected from —H, R′ is selected from —H;
R is selected from —CH₃, R′ is selected from —H; or
R is selected from 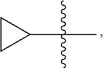 [SMILES: *C1CC1]
R′ is selected from —H; 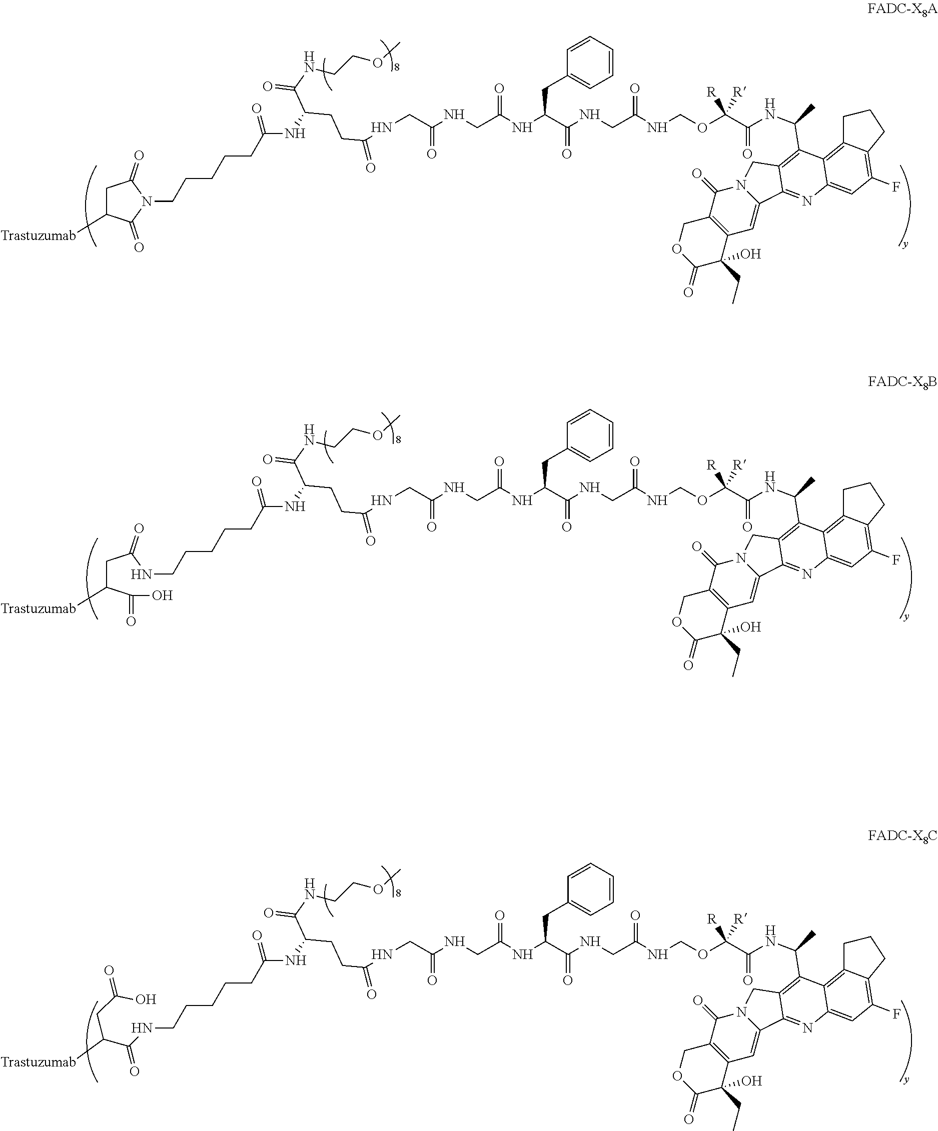
wherein, R is selected from —H, R′ is selected from —H;
R is selected from —CH₃, R′ is selected from —H;
R is selected from 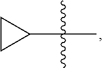 [SMILES: *C1CC1]
R′ is selected from —H; or
R is selected from —H, R′ is selected from 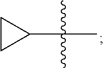 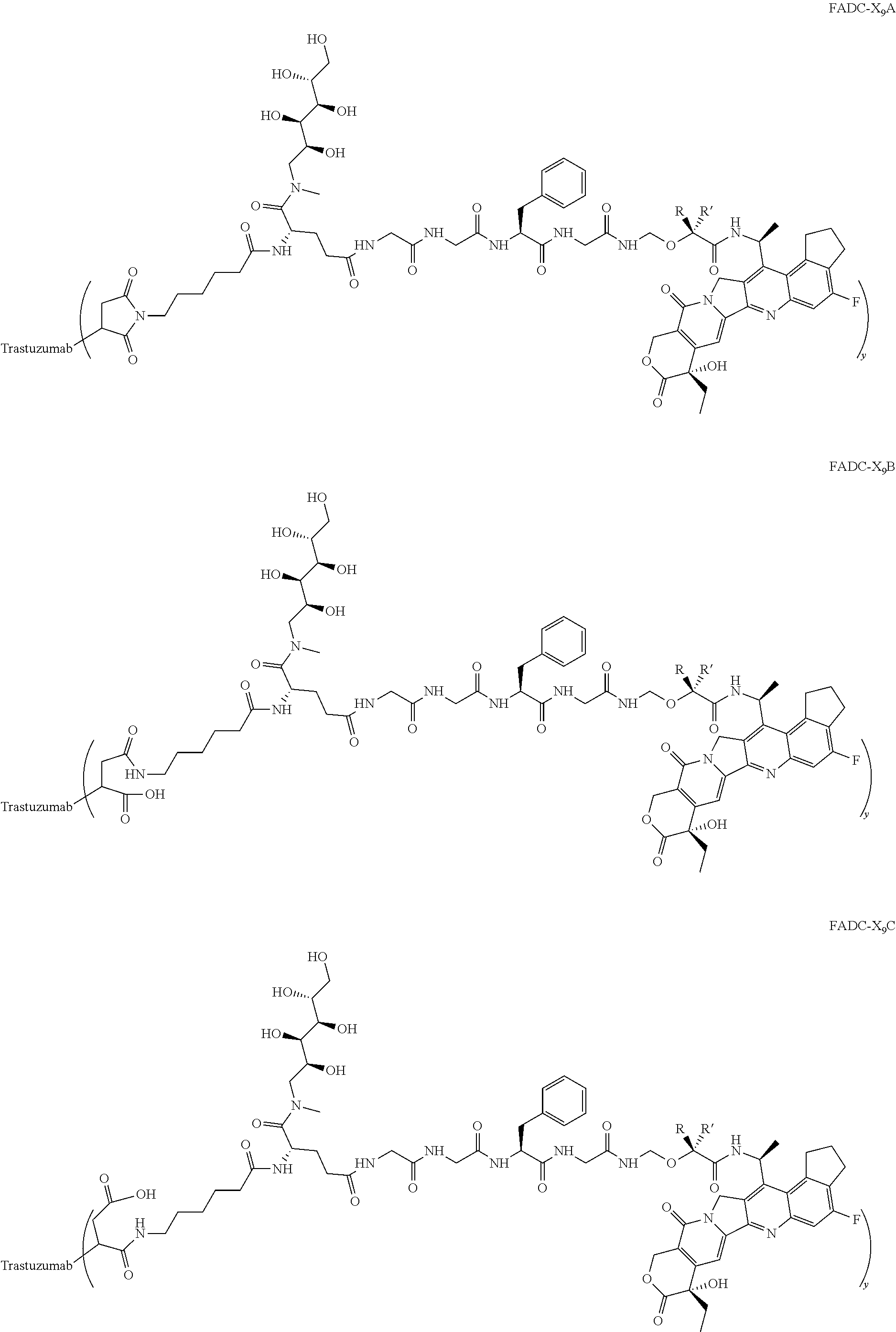
wherein, R is selected from —H, R′ is selected from —H;
R is selected from —CH₃, R′ is selected from —H; or
R is selected from 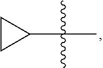 [SMILES: *C1CC1]
R′ is selected from —H; 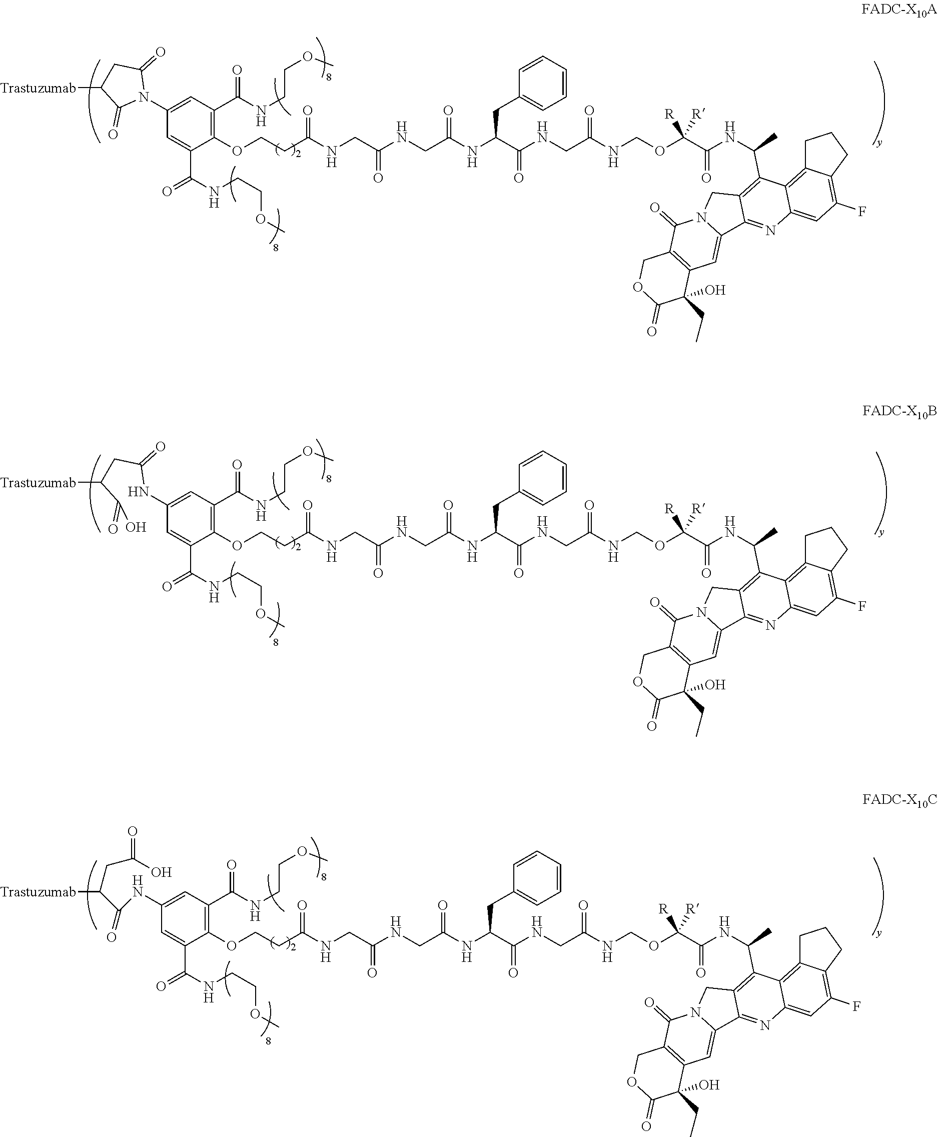
wherein, R is selected from —H, R′ is selected from —H;
R is selected from 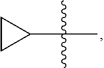 [SMILES: *C1CC1]
R′ is selected from —H; or
R is selected from —H, R′ is selected from 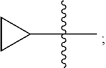 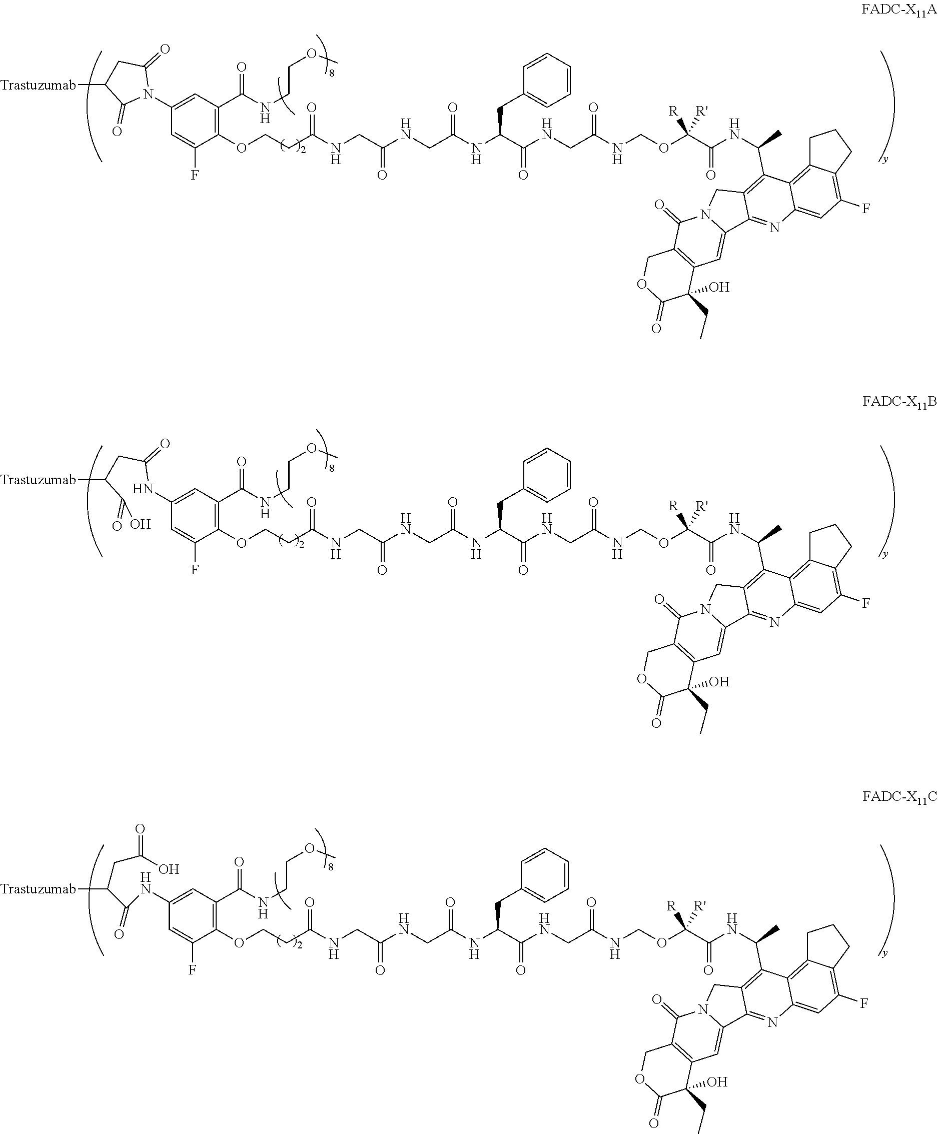
wherein, R is selected from —H, R′ is selected from —H;
R is selected from 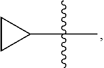 [SMILES: *C1CC1]
R′ is selected from —H; or
R is selected from —H, R′ is selected from 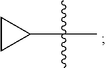 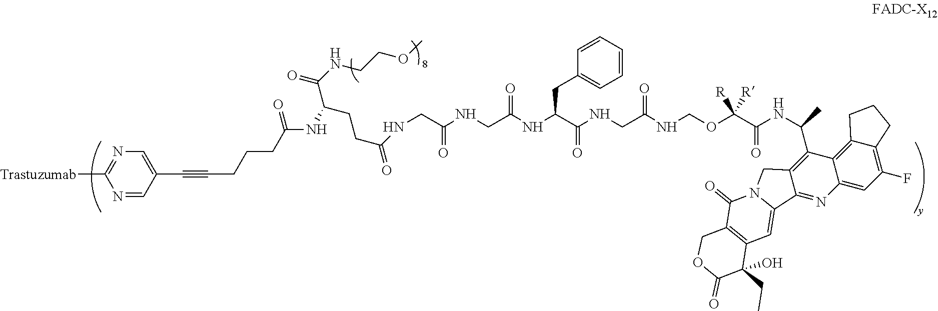
wherein, R is selected from —H, R′ is selected from —H;
R is selected from 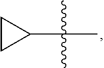 [SMILES: *C1CC1]
R′ is selected from —H; or
R is selected from —H, R′ is selected from 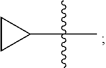 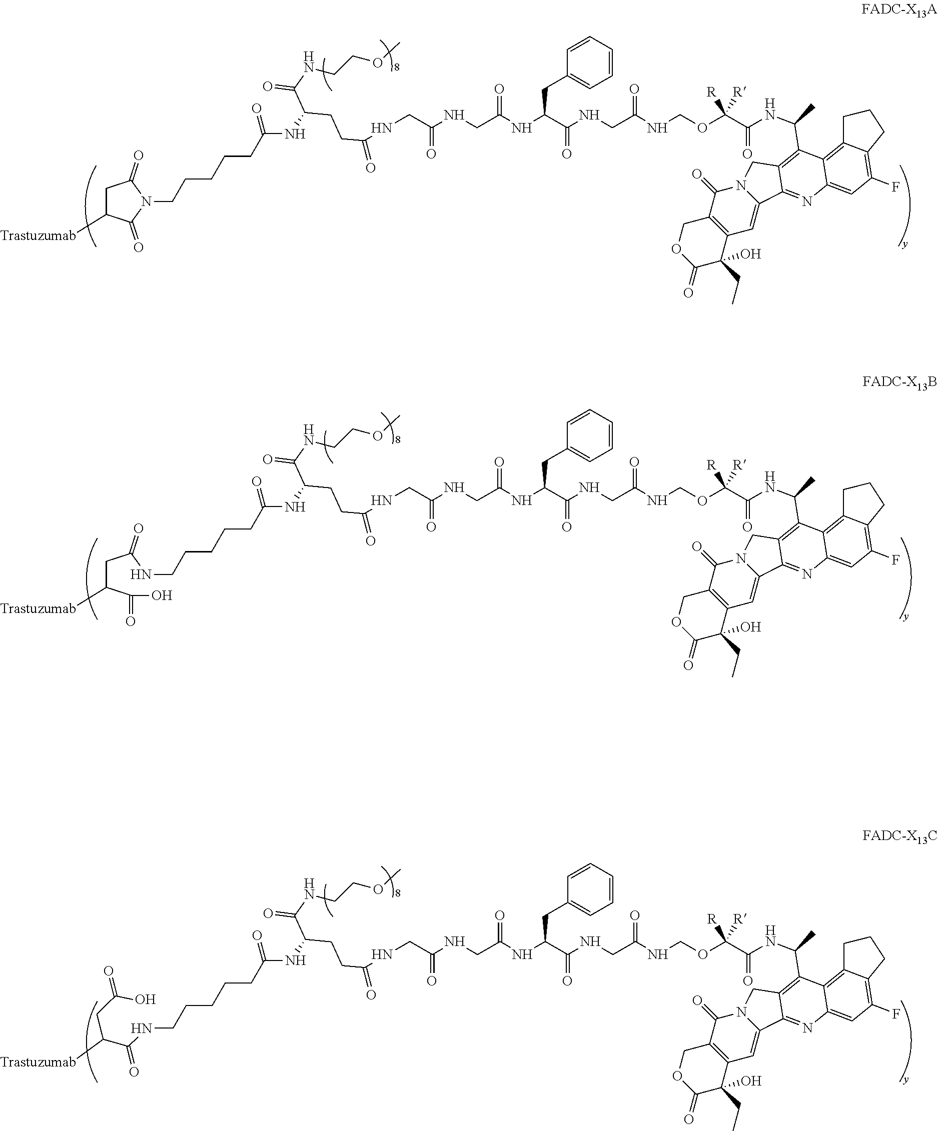
wherein, R is selected from —H, R′ is selected from —H;
R is selected from 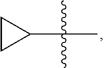 [SMILES: *C1CC1]
R′ is selected from —H; or
R is selected from —H, R′ is selected from 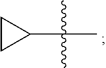 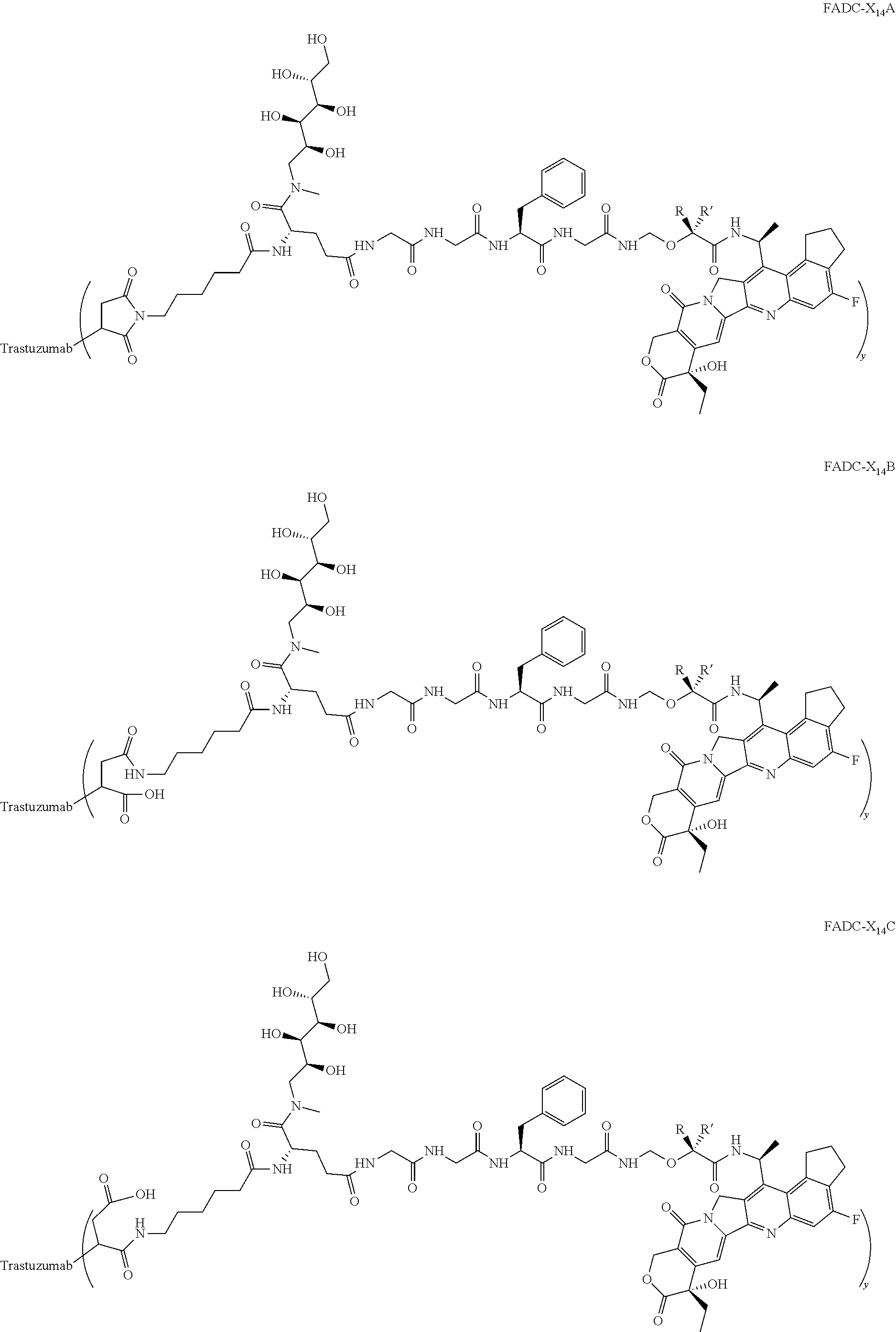
wherein, R is selected from —H, R′ is selected from —H;
R is selected from 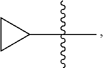 [SMILES: *C1CC1]
R′ is selected from —H; or
R is selected from —H, R′ is selected from 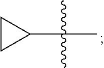 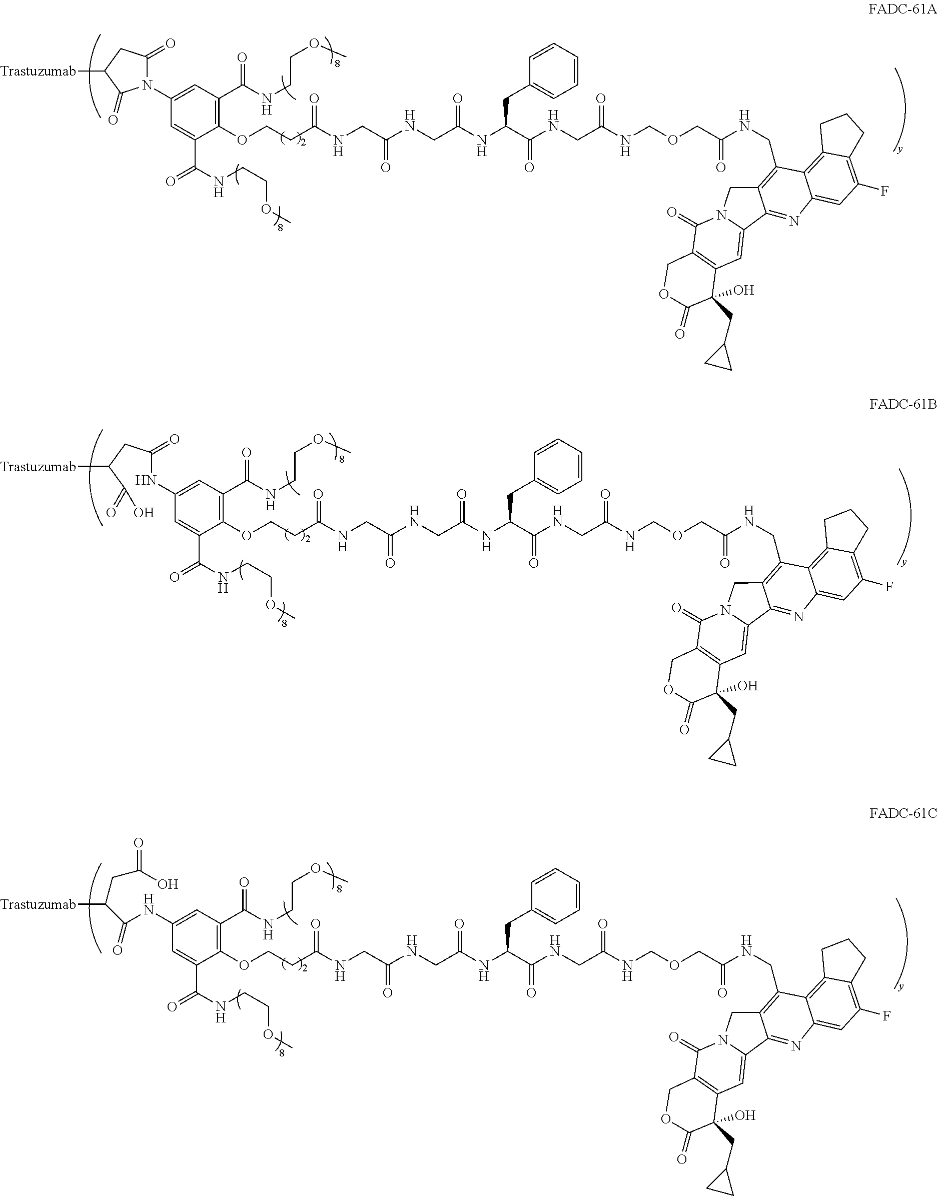 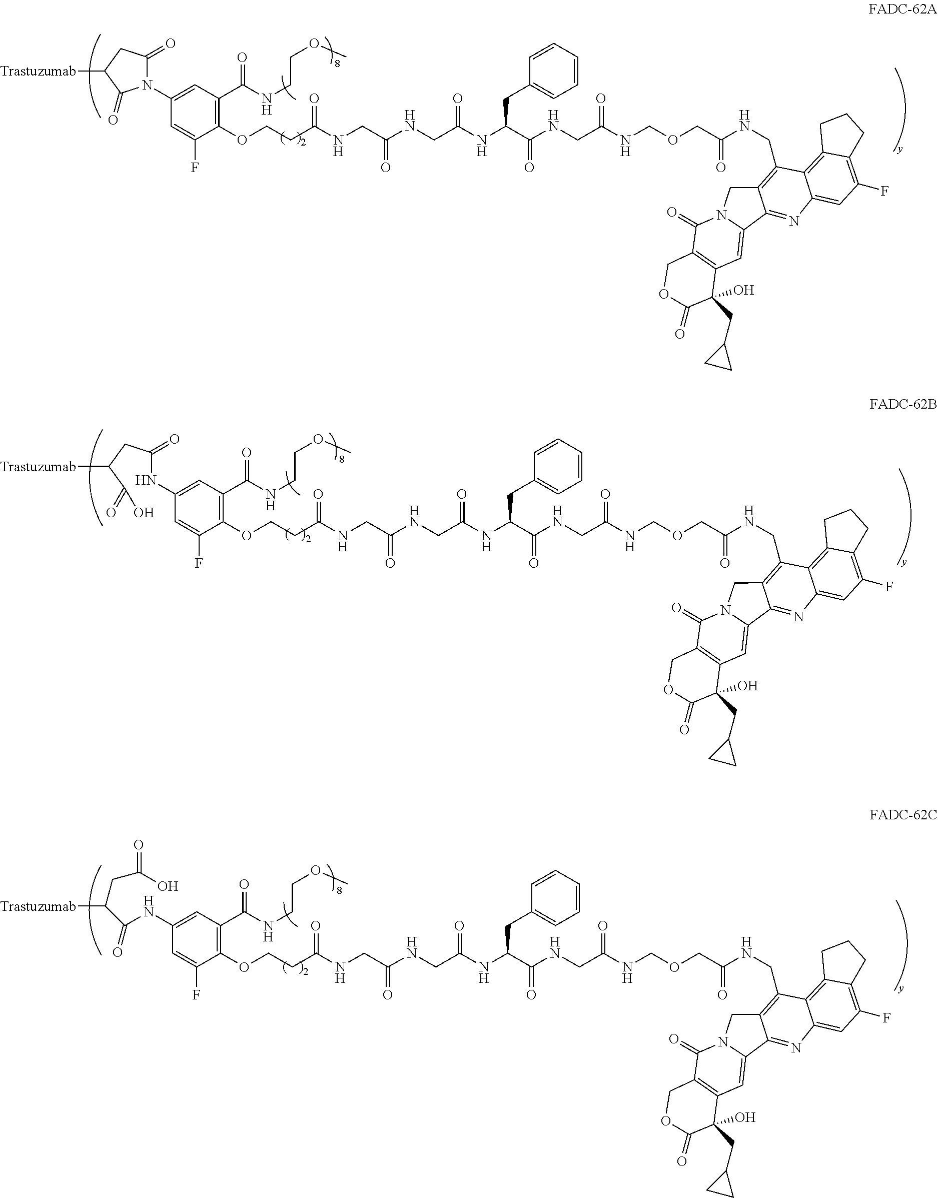 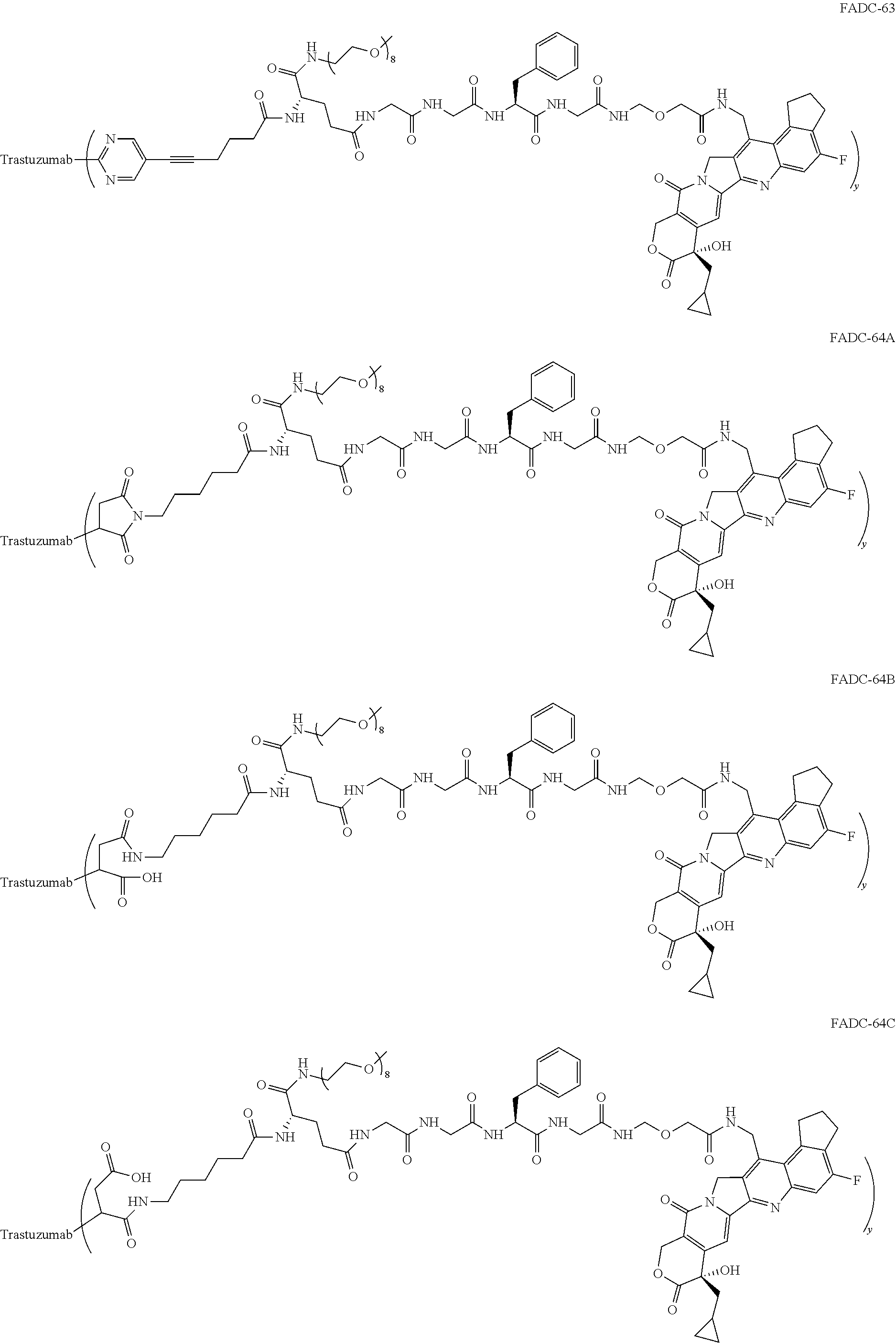 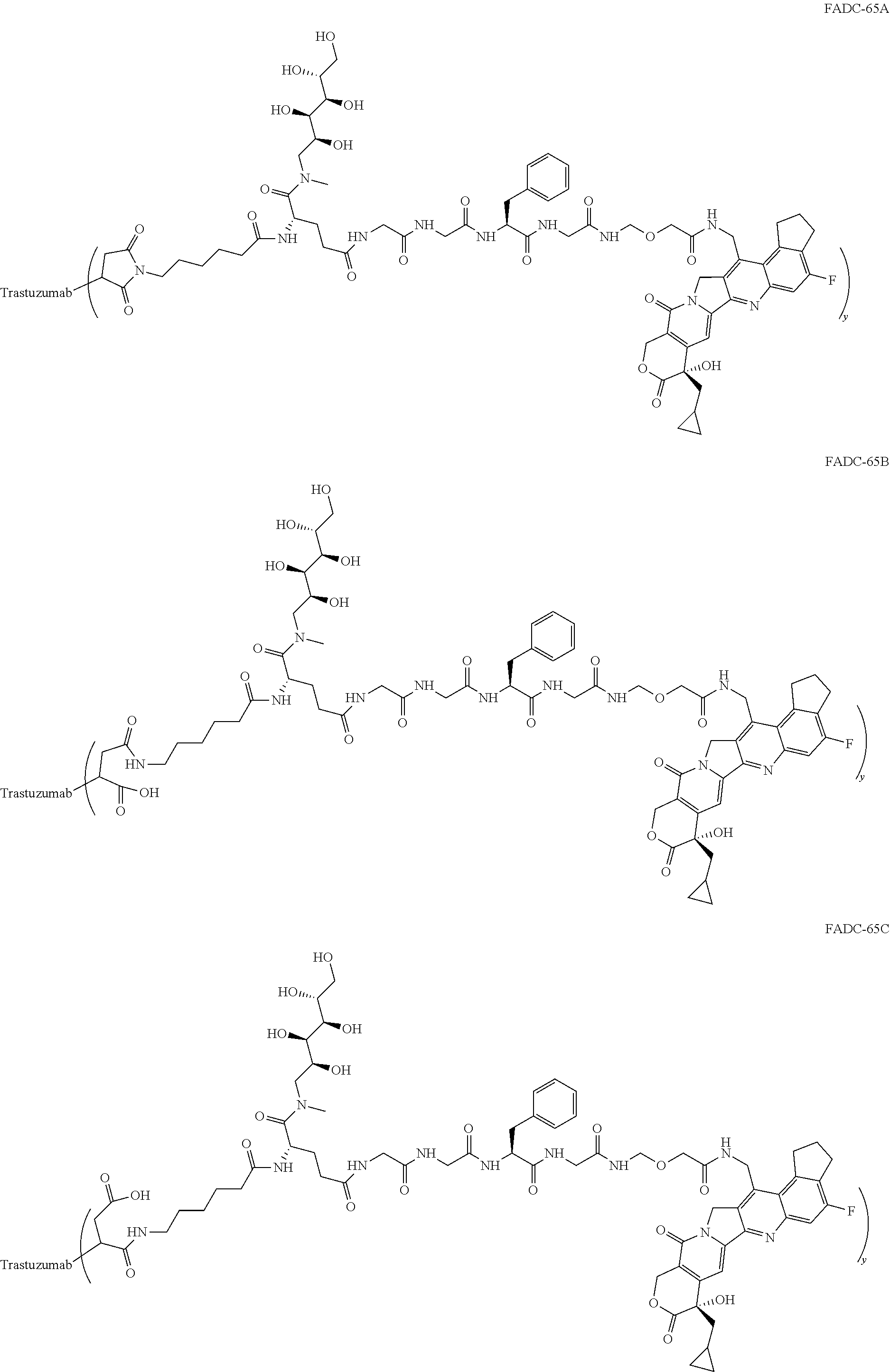 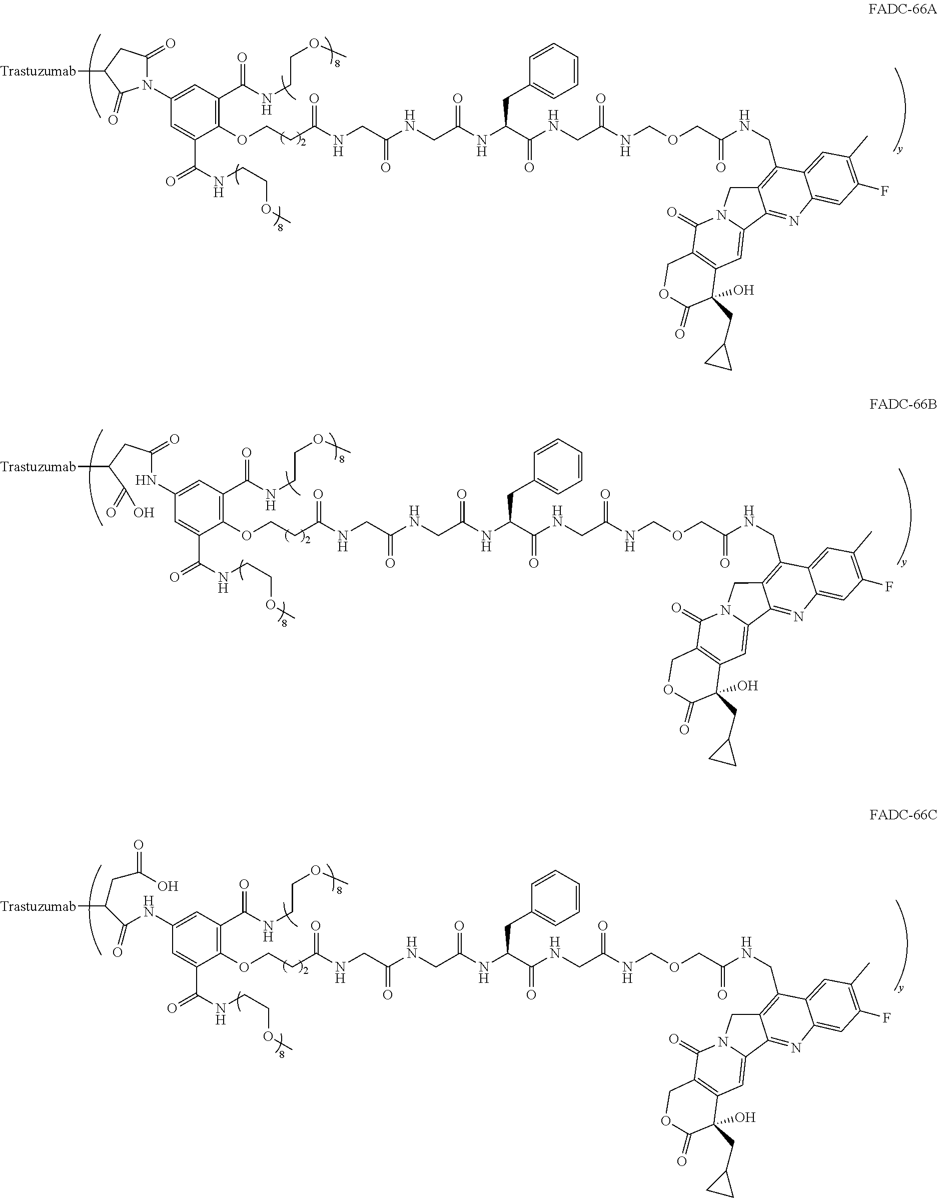 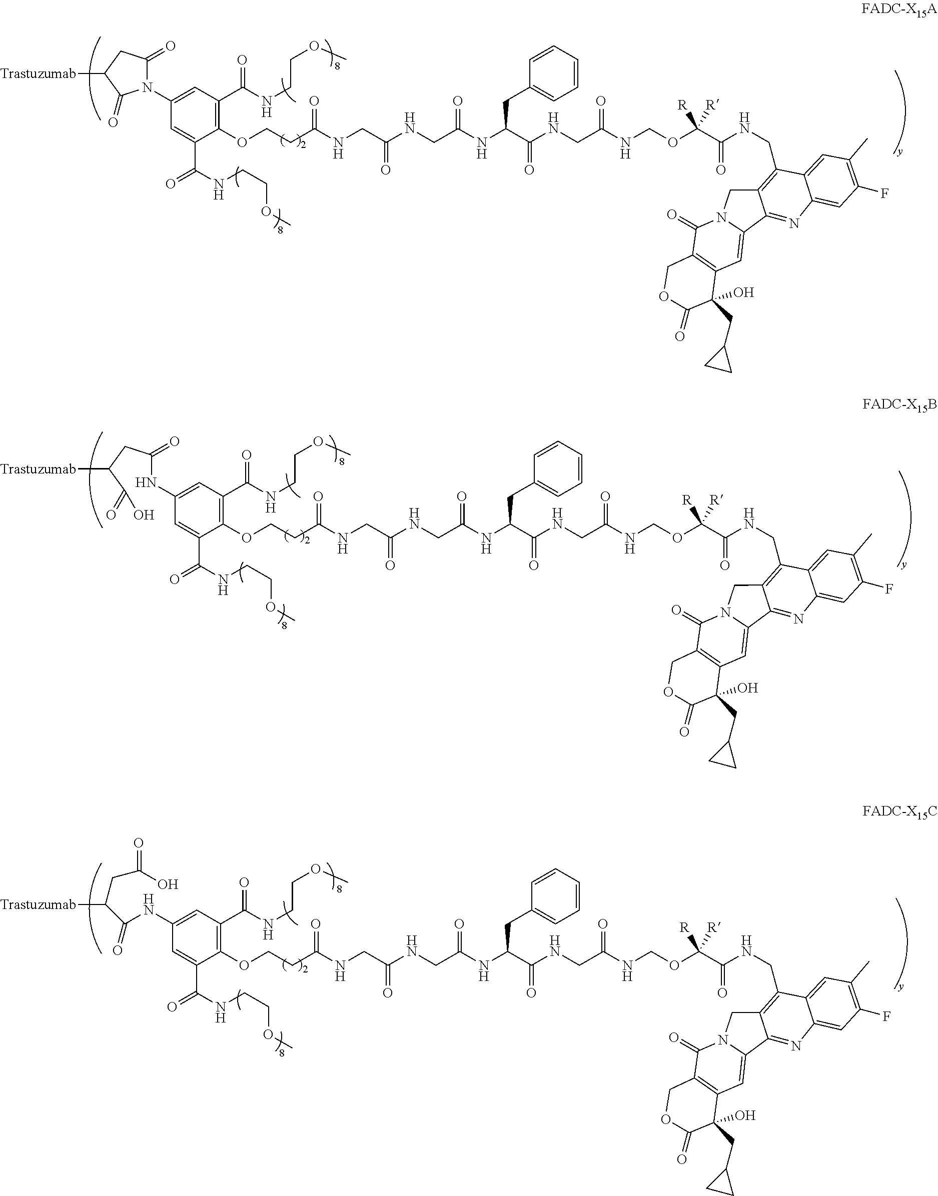
wherein, R is selected from —CH₃, R′ is selected from —H;
R is selected from —H, R′ is selected from —CH₃;
R is selected from 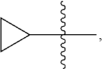 [SMILES: *C1CC1]
R′ is selected from —H; or
R is selected from —H, R′ is selected from 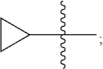 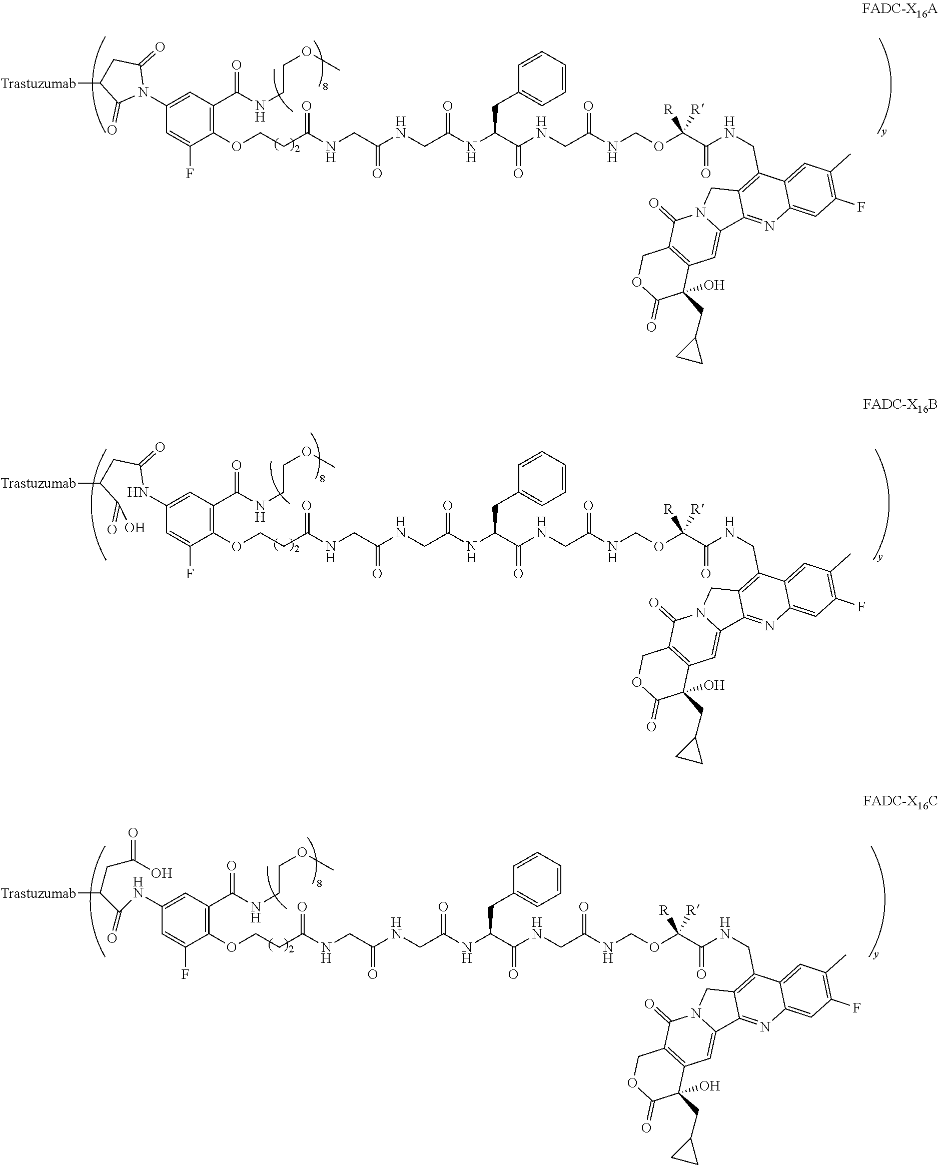
wherein, R is selected from —H, R′ is selected from —H;
R is selected from —CH₃, R′ is selected from —H;
R is selected from —H, R′ is selected from —CH₃;
R is selected from 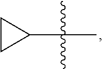 [SMILES: *C1CC1]
R′ is selected from —H; or
R is selected from —H, R′ is selected from 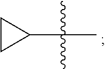 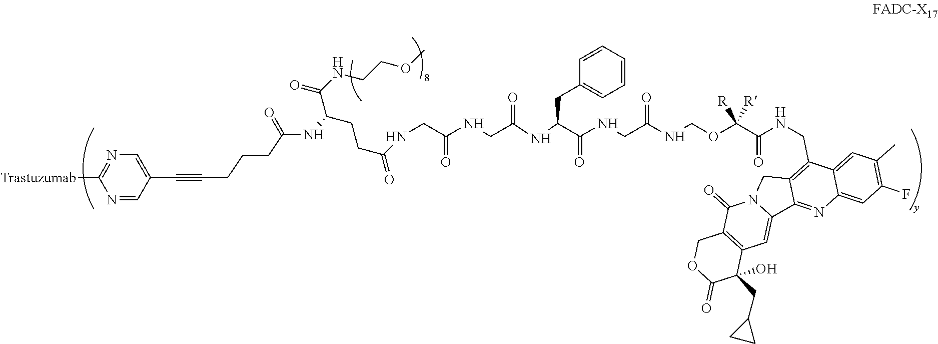
wherein, R is selected from —H, R′ is selected from —H;
R is selected from —CH₃, R′ is selected from —H;
R is selected from —H, R′ is selected from —CH₃;
R is selected from 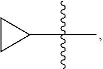 [SMILES: *C1CC1]
R′ is selected from —H; or
R is selected from —H, R′ is selected from >; 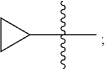 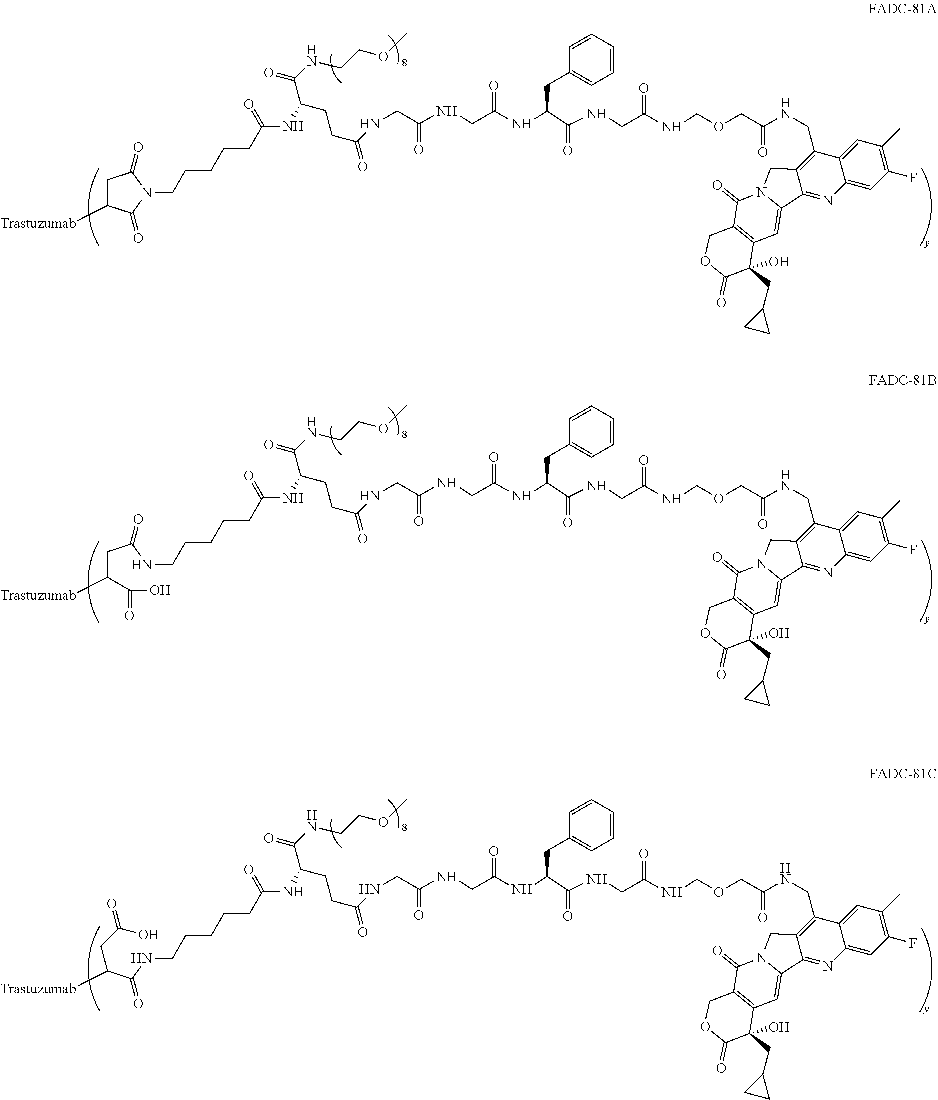 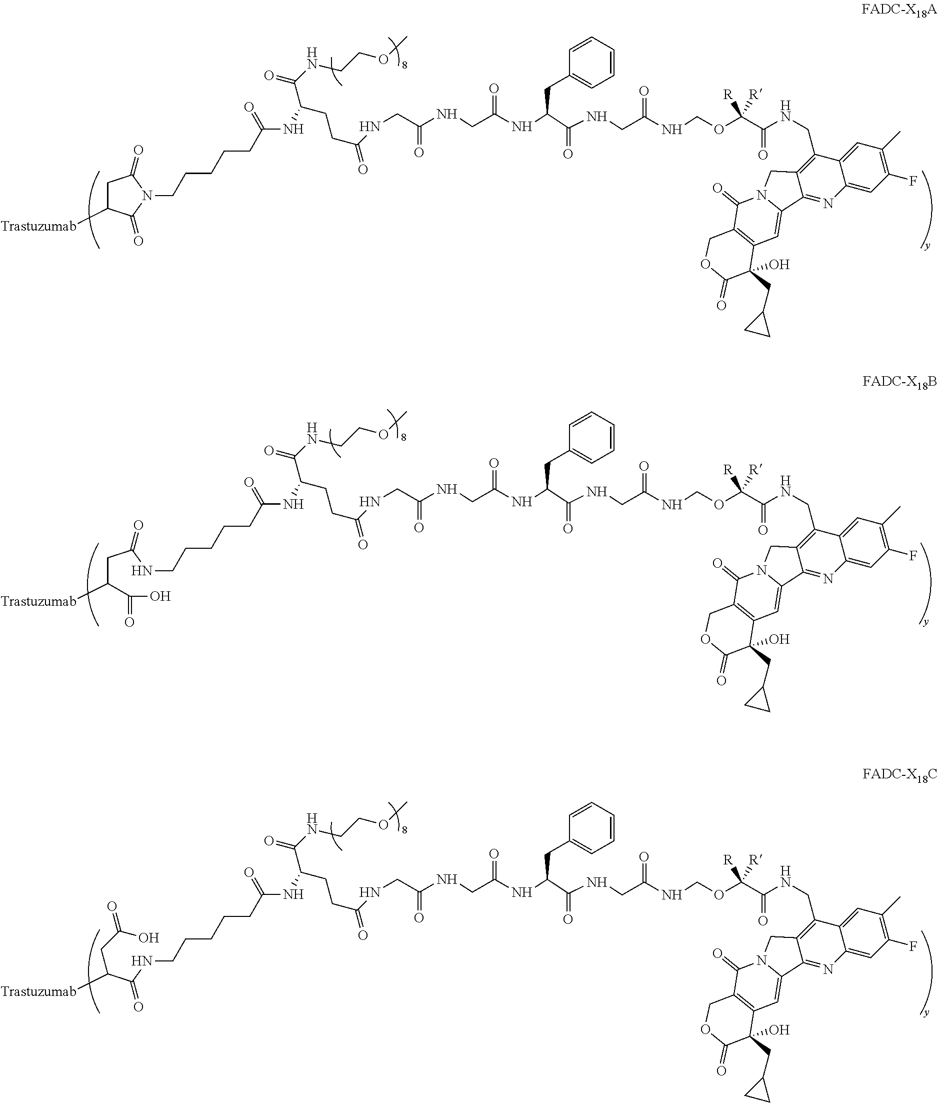
wherein, R is selected from —CH₃, R′ is selected from —H;
R is selected from —H, R′ is selected from —CH₃;
R is selected from 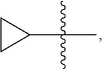 [SMILES: *C1CC1]
R′ is selected from —H; or
R is selected from —H, R′ is selected from 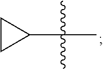 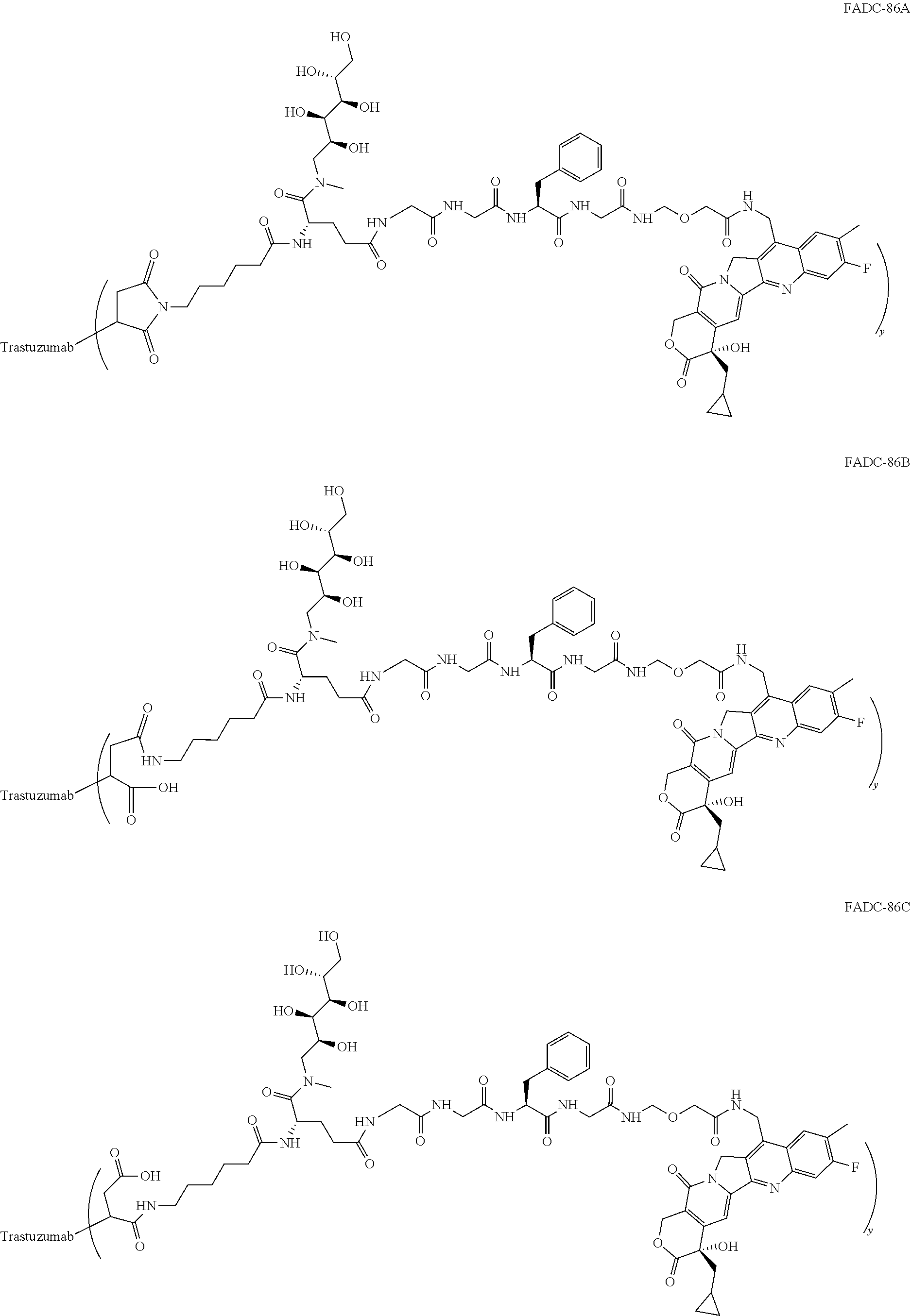 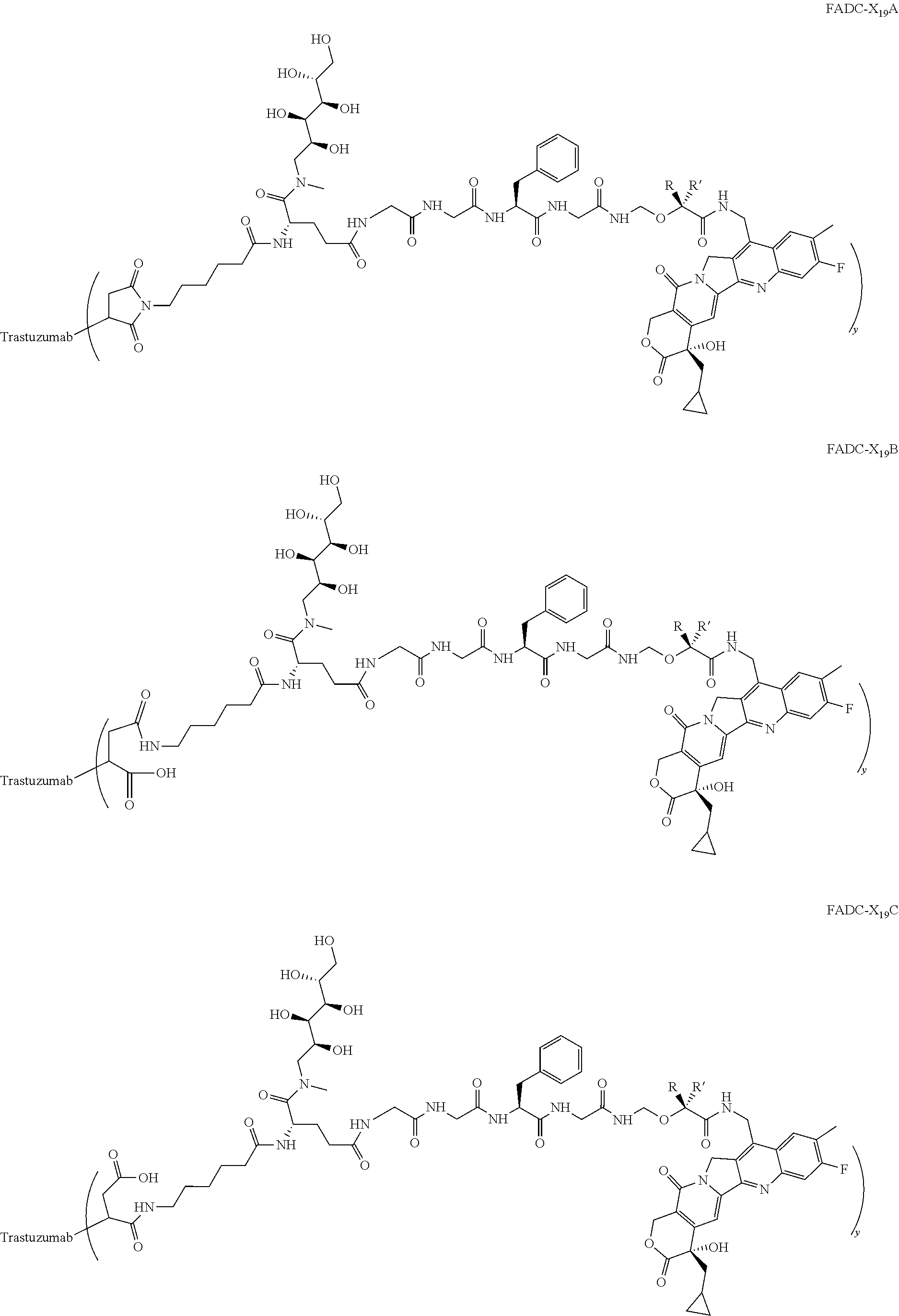
wherein, R is selected from —CH₃, R′ is selected from —H;
R is selected from —H, R′ is selected from —CH₃;
R is selected from 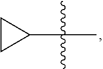 [SMILES: *C1CC1]
R′ is selected from —H; or
R is selected from —H, R′ is selected from 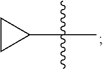 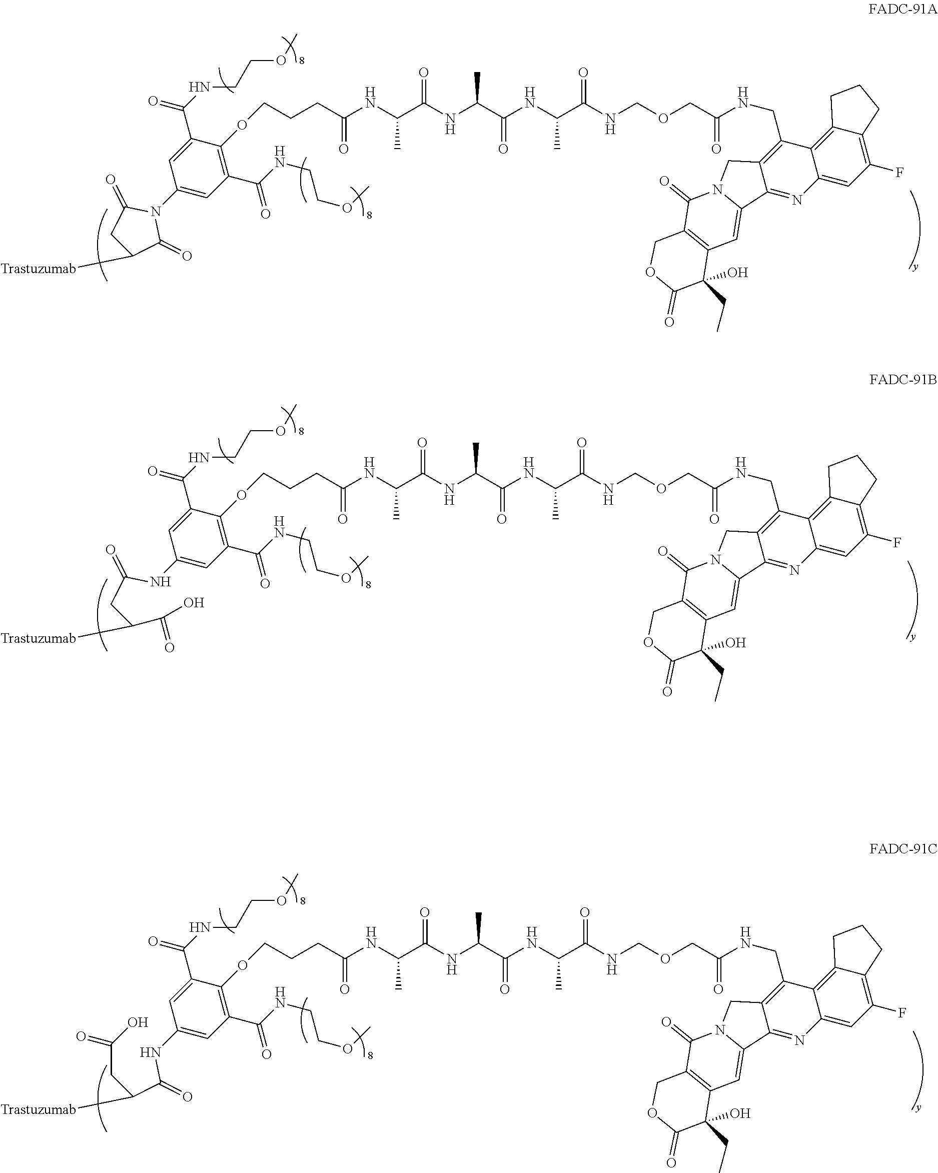 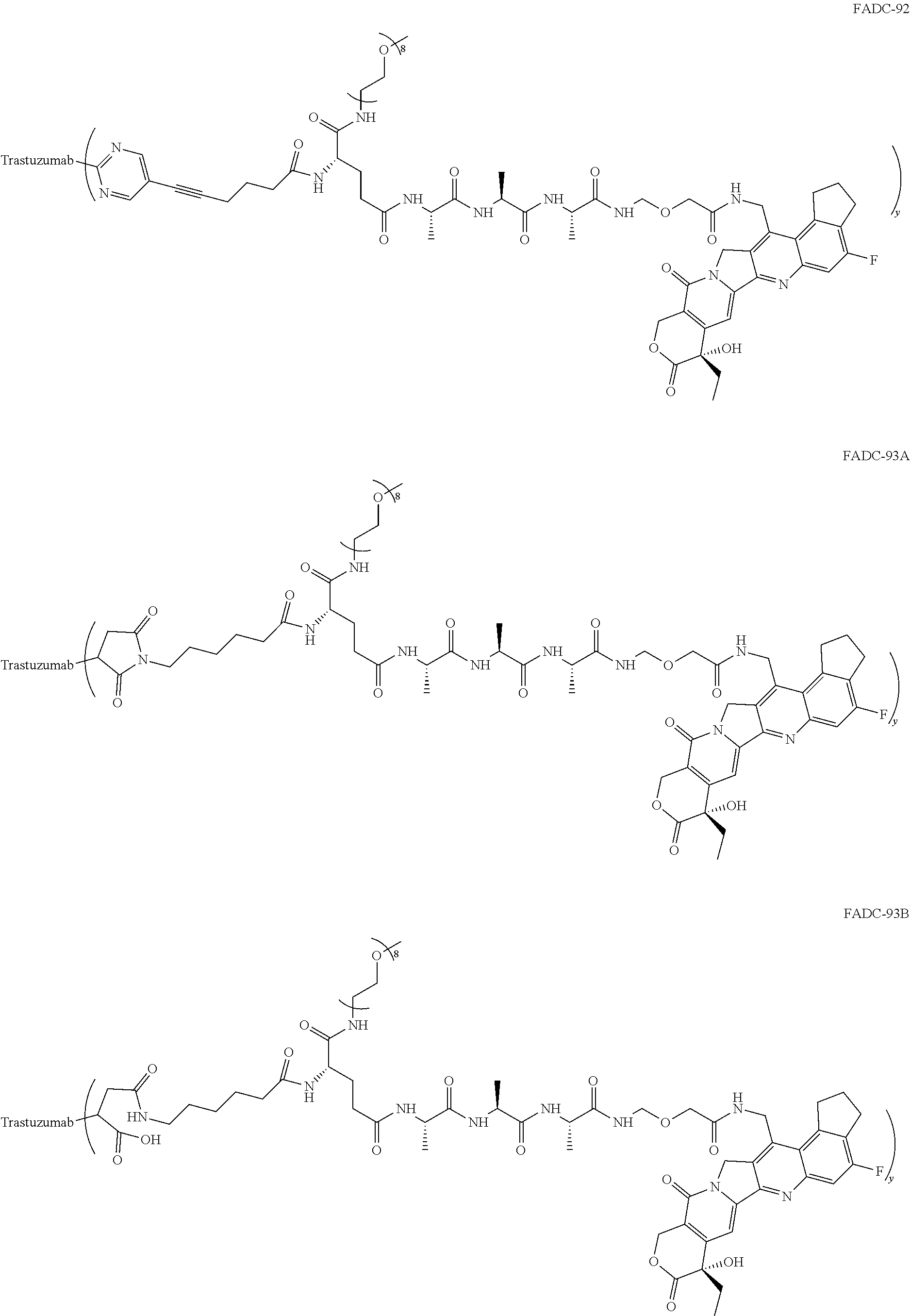 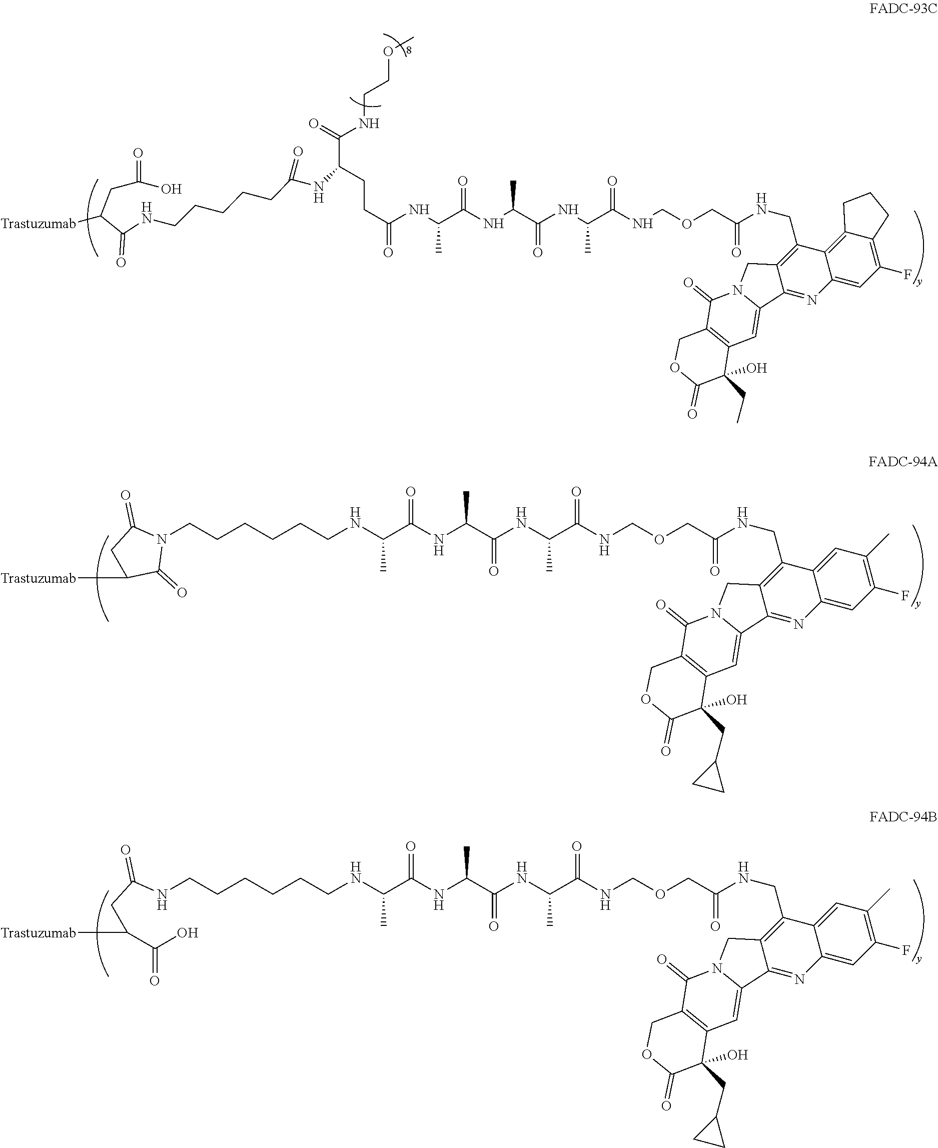 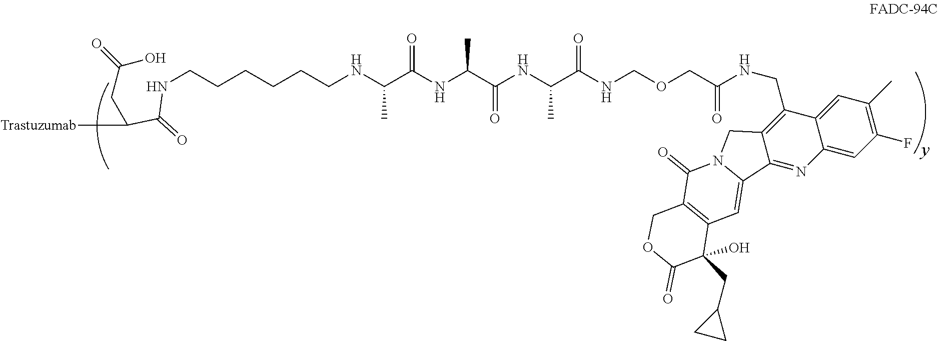 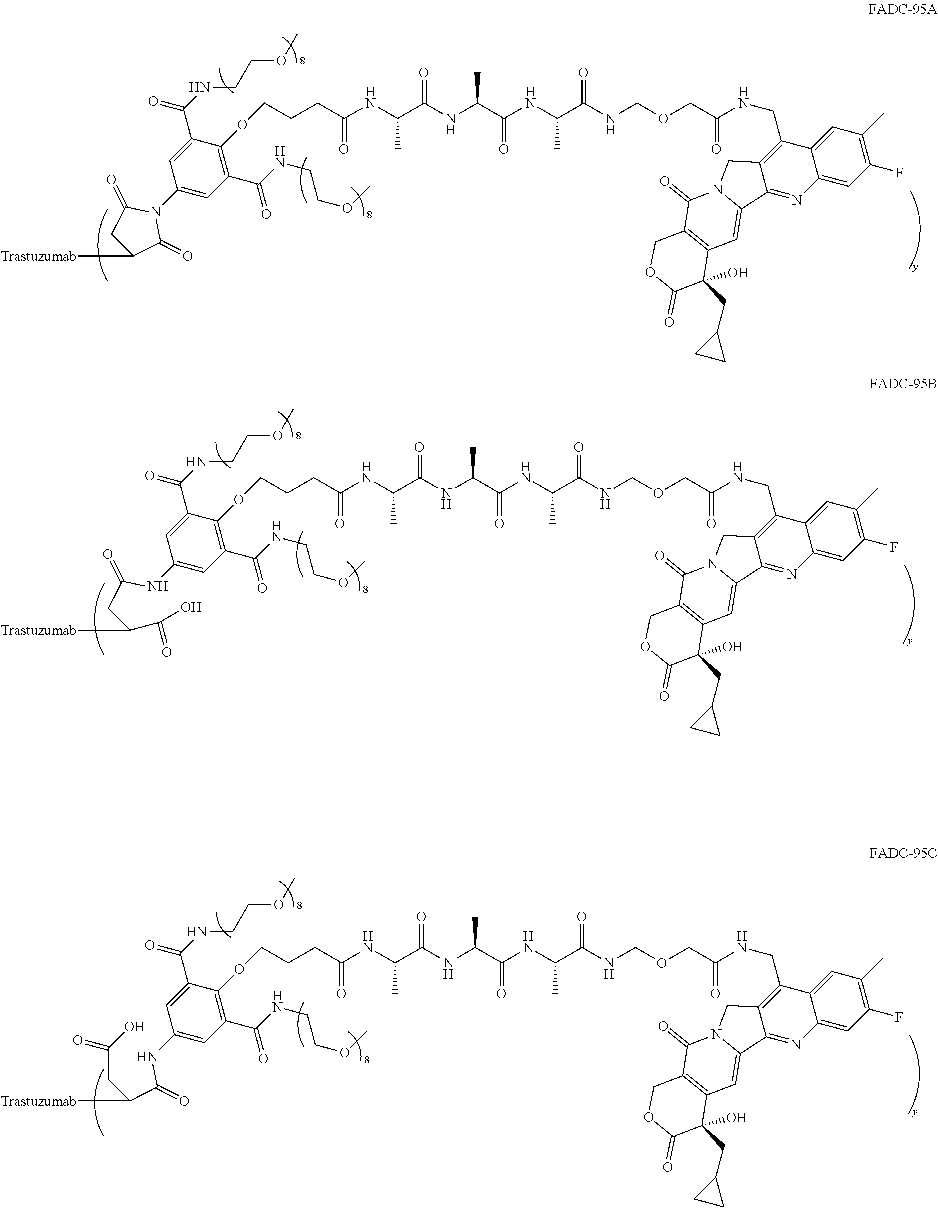 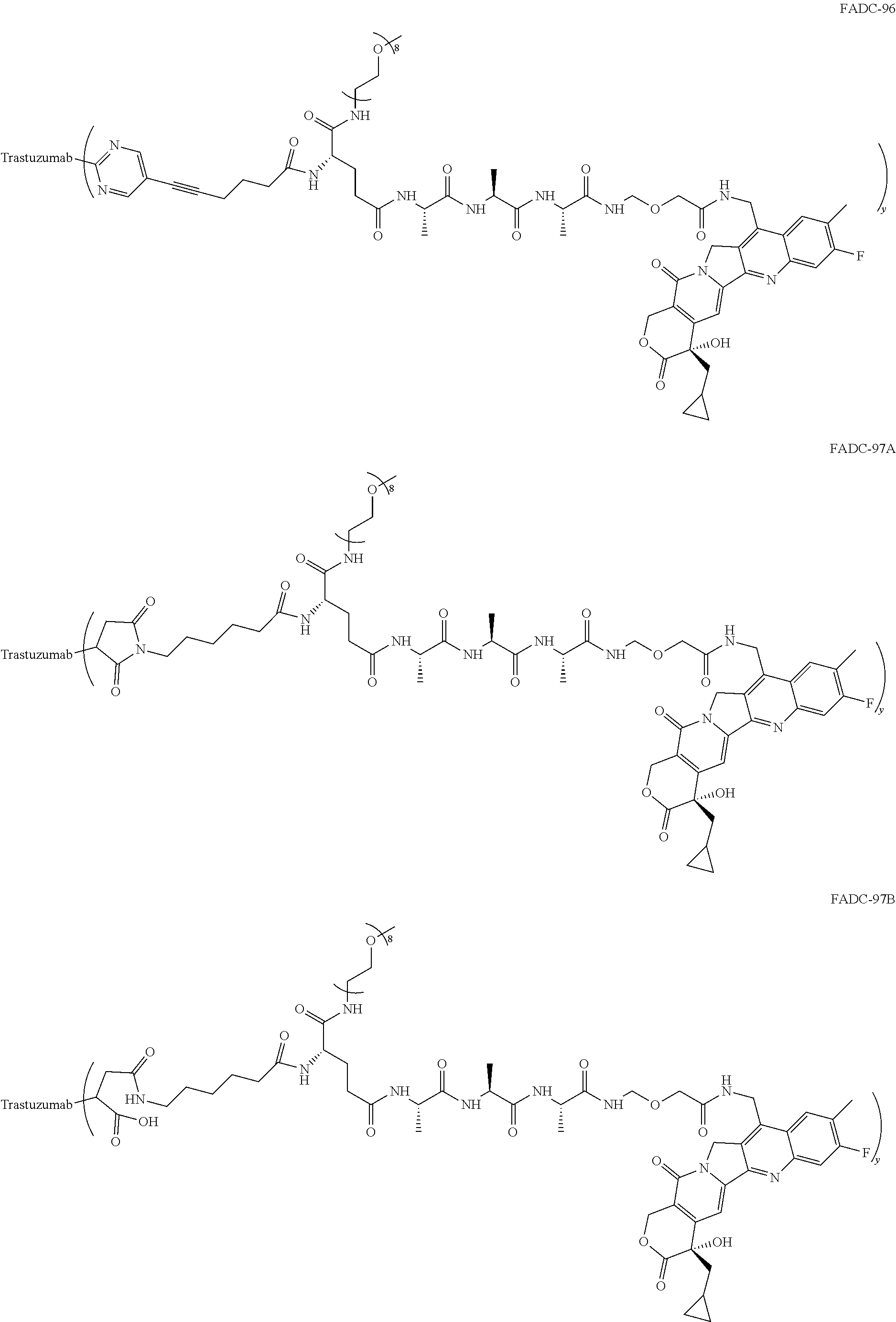 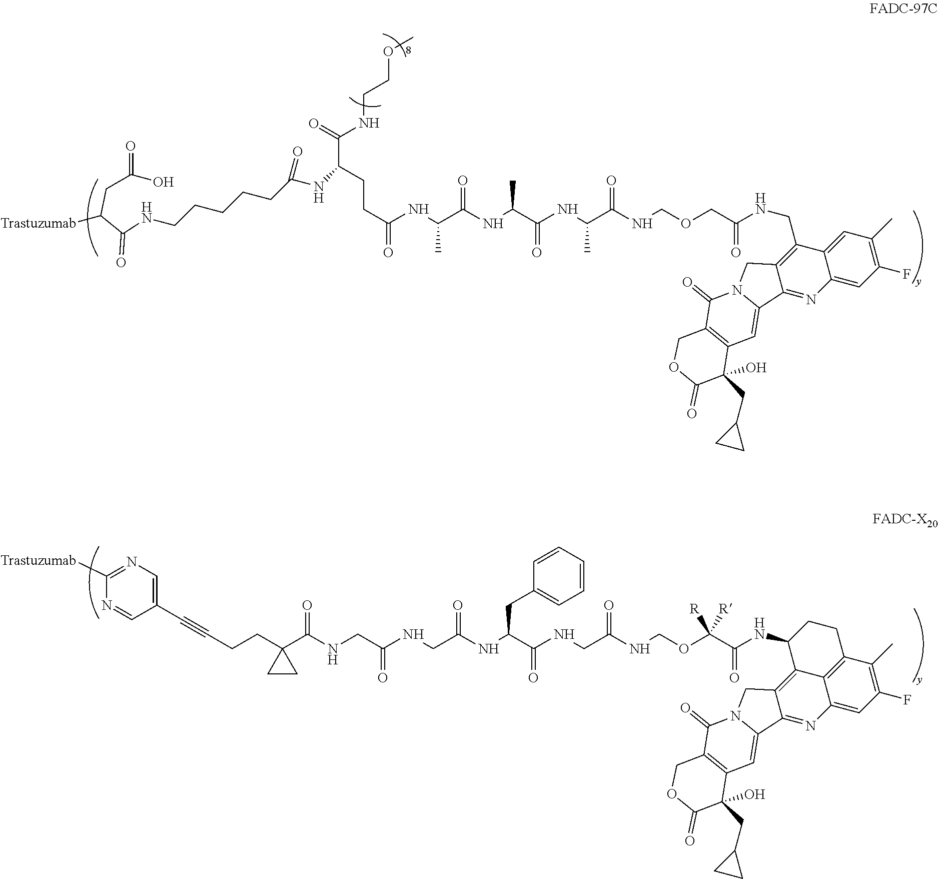
wherein, R═—H, R′═—H;
R═—CH₃, R′═—H;
R═—H, R′═—CH₃;
R= 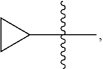 [SMILES: *C1CC1]
R′═—H; or
R═—H, R′= 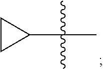 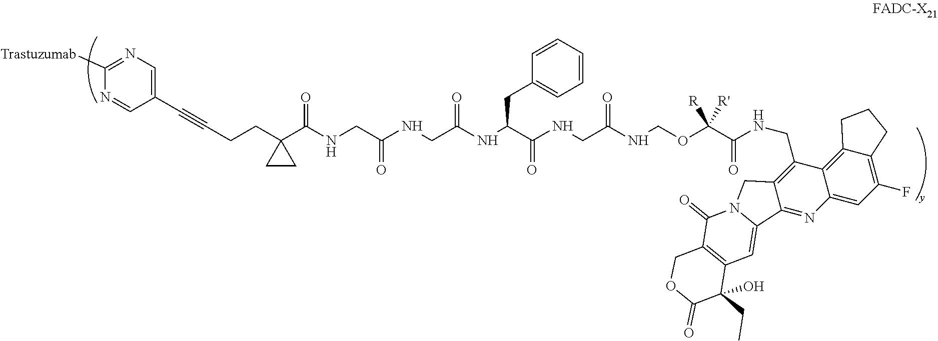
wherein, R═—H, R′═—H;
R═—CH₃, R′═—H; or
R═—H, R′═—CH₃; 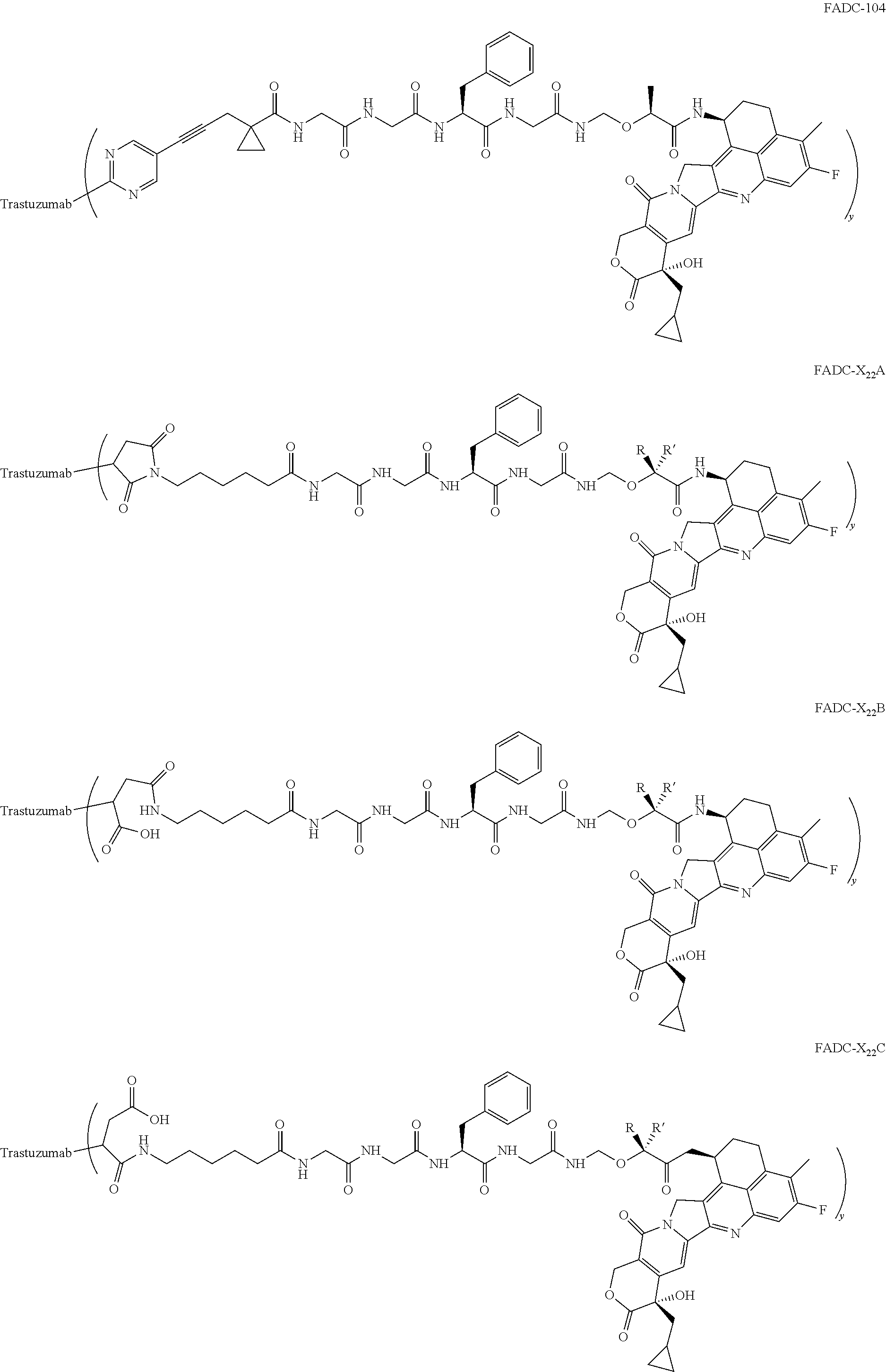
wherein, R═—H, R′═—CH₃; or
R═—CH₃, R′═—H; 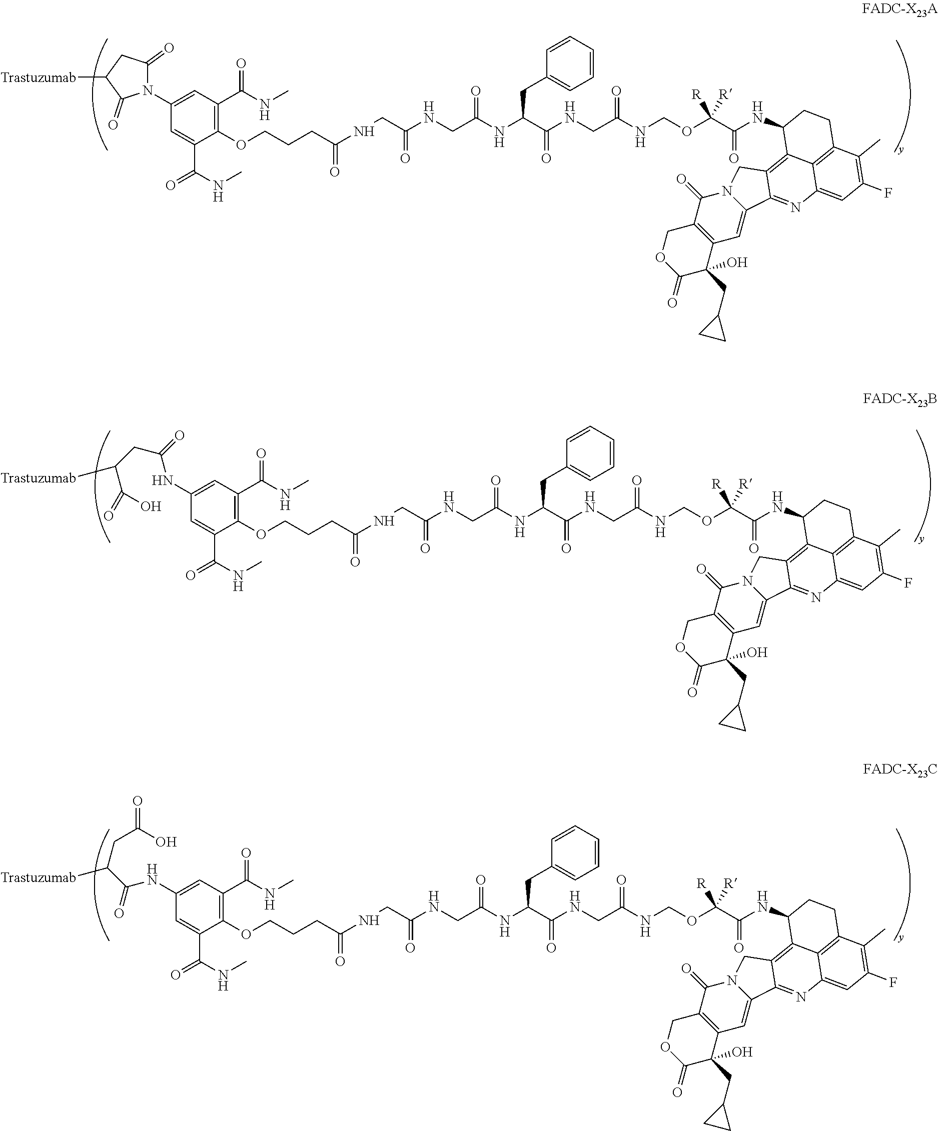
wherein, R═—H, R′═—H;
R═—CH₃, R′═—H;
R= 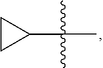 [SMILES: *C1CC1]
R′═—H; or
R═—H, R′= 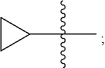 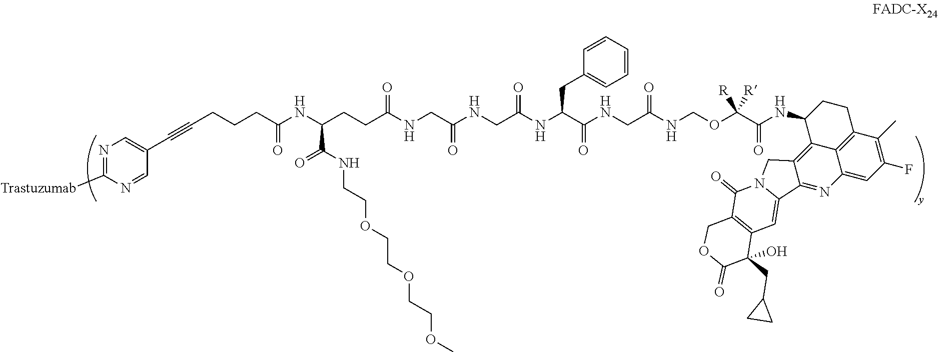
wherein, R═—H, R′═—H;
R═—CH₃, R′═—H;
R═—H, R′═—CH₃;
R= 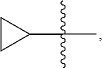 [SMILES: *C1CC1]
R′═—H; or
R═—H, R′=

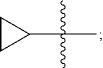 [SMILES: *C1CC1]

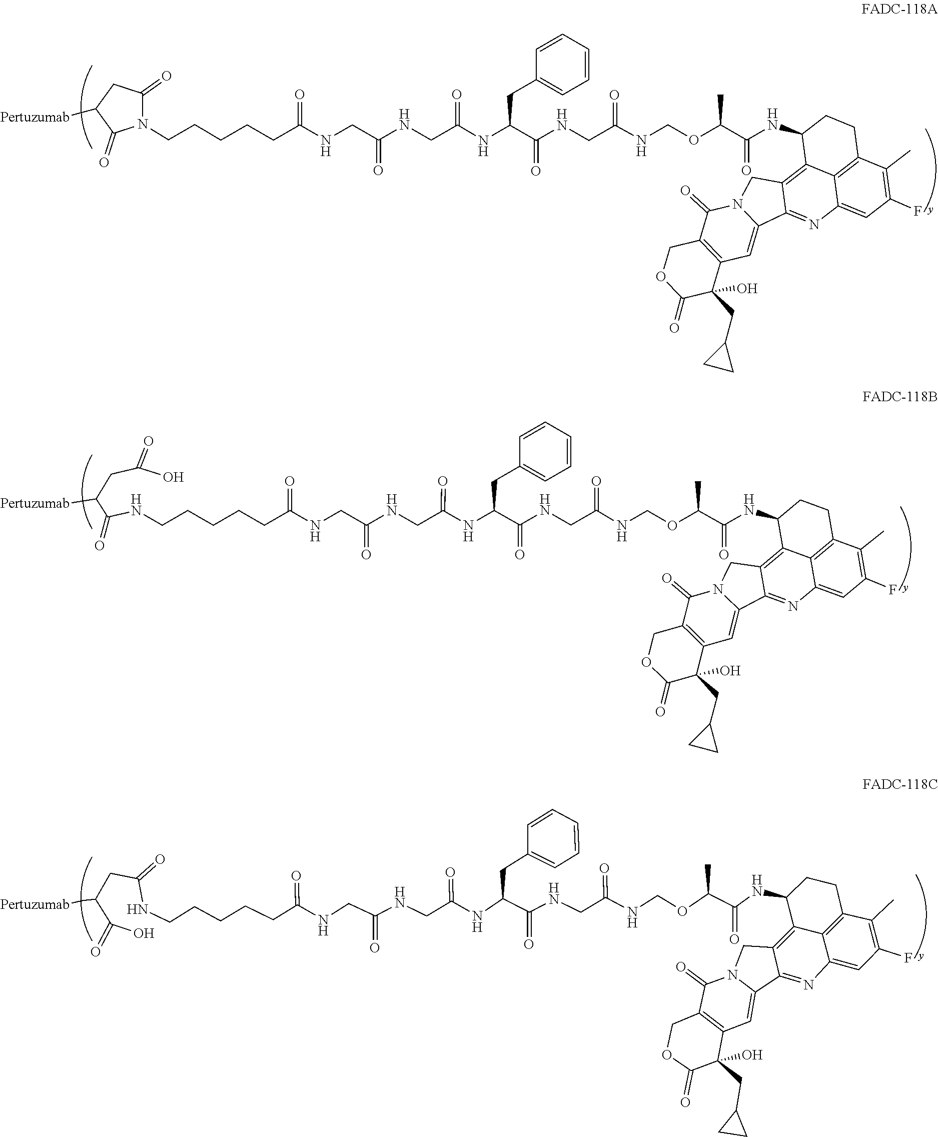

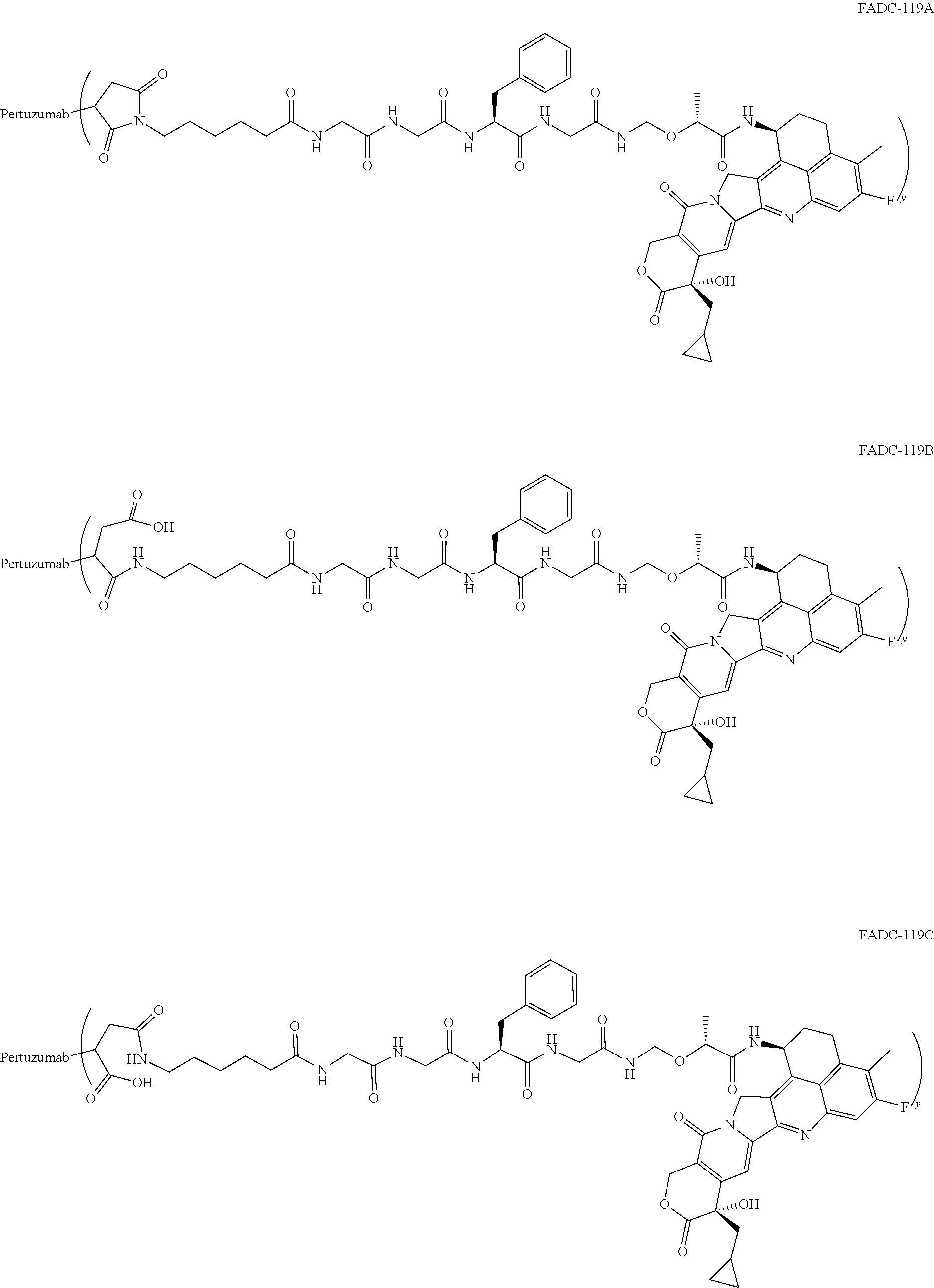

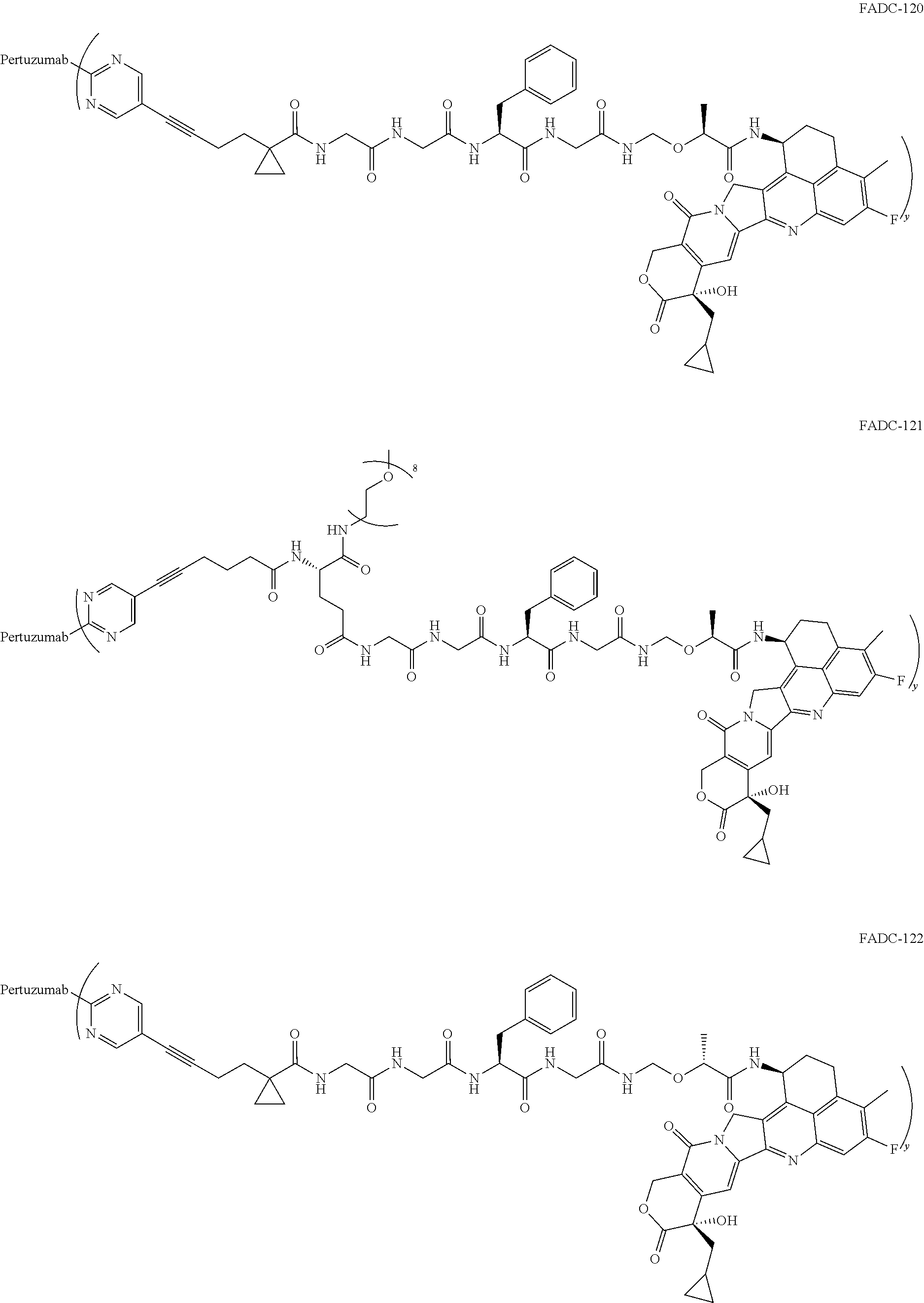

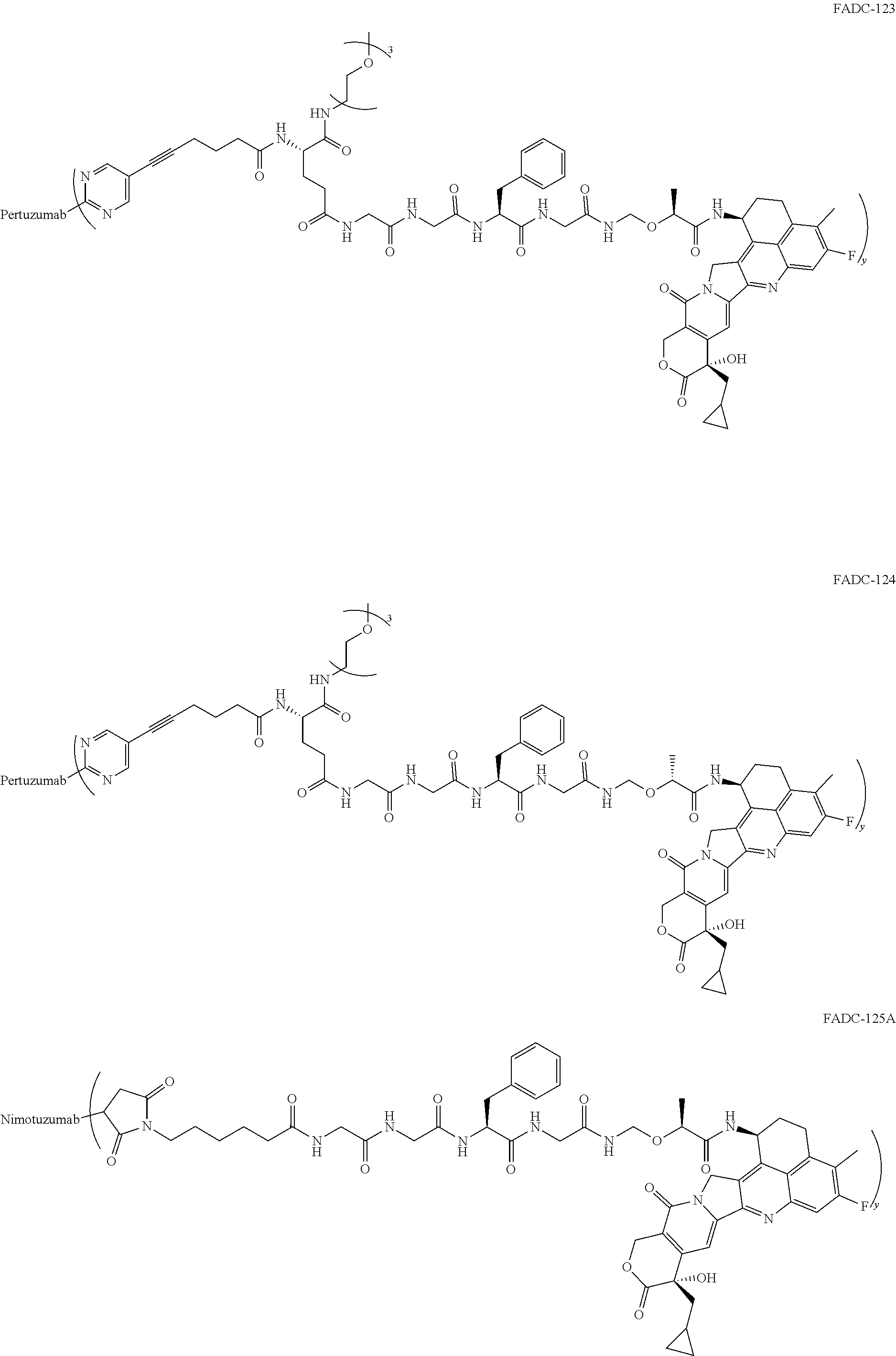

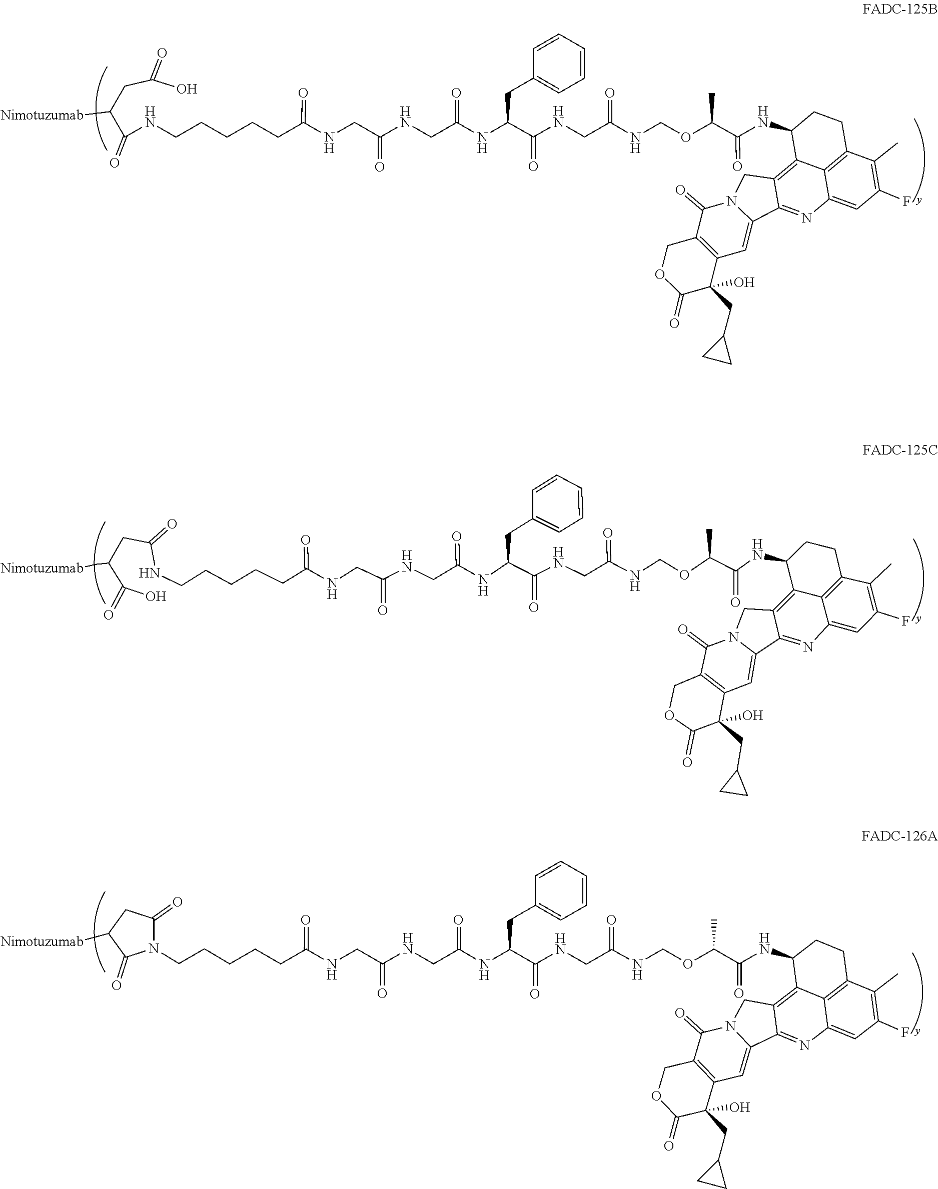

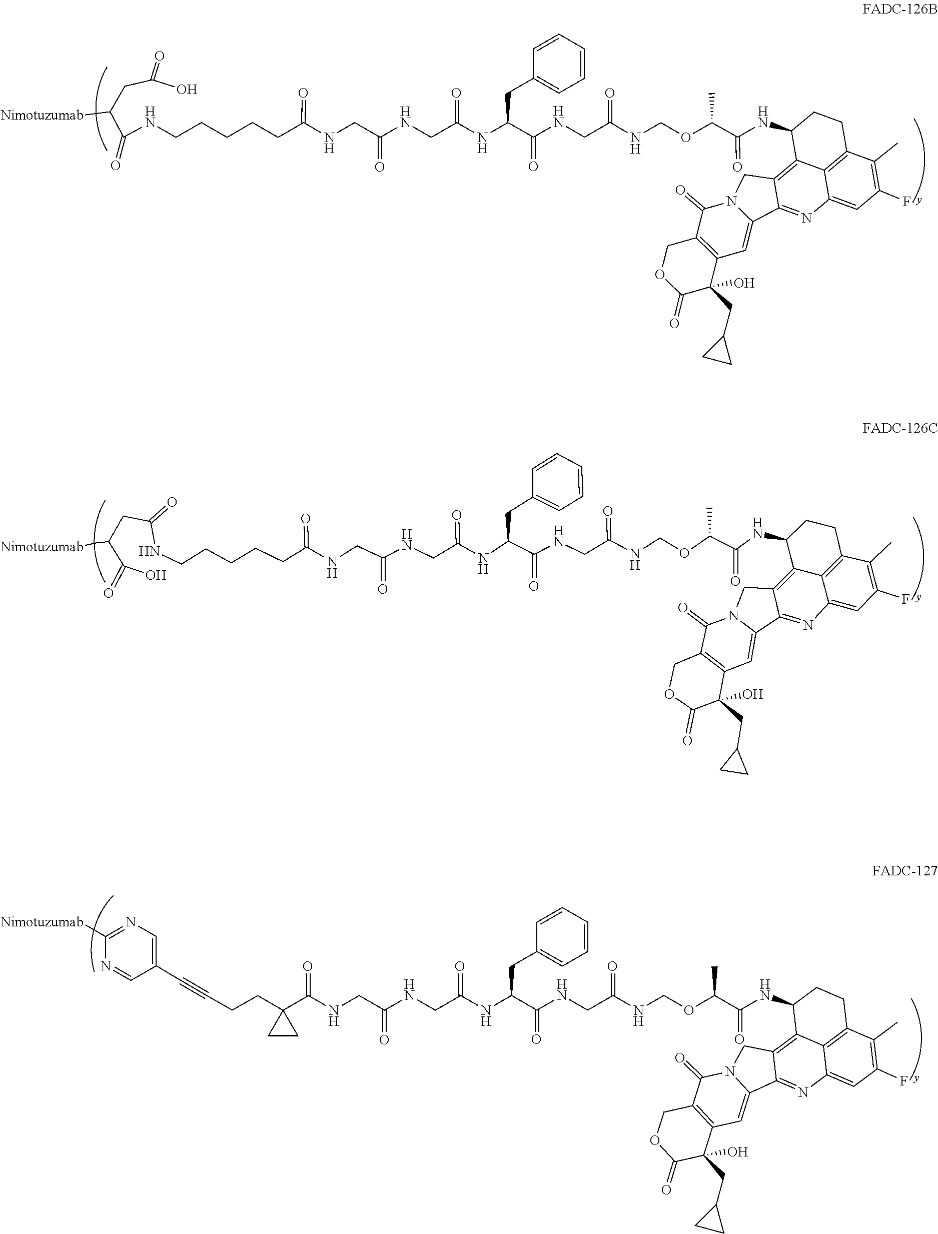

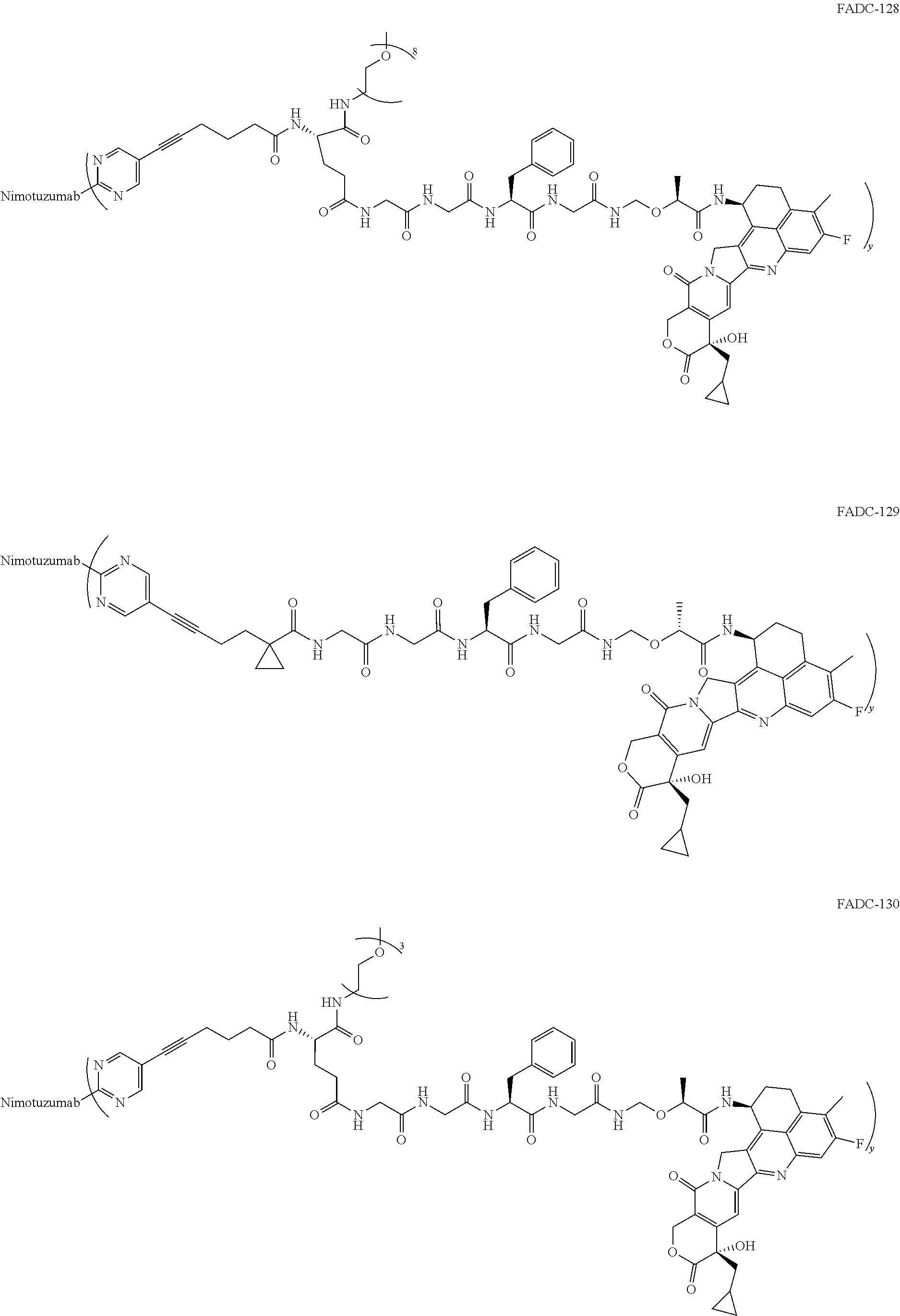

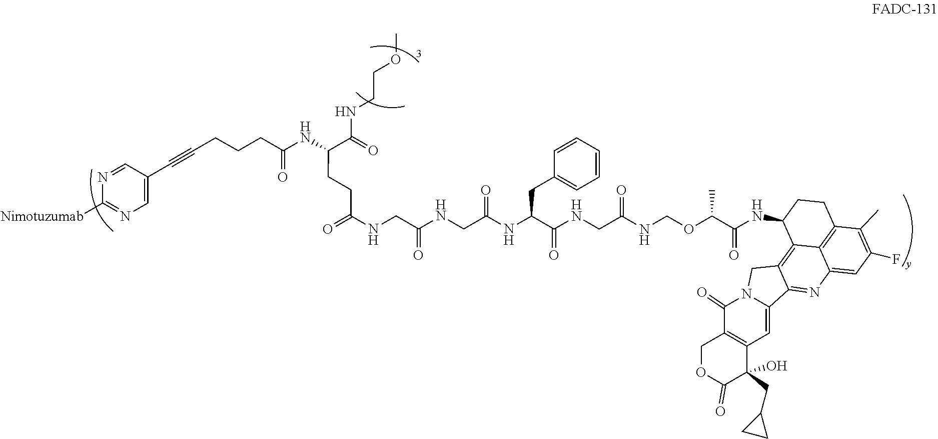

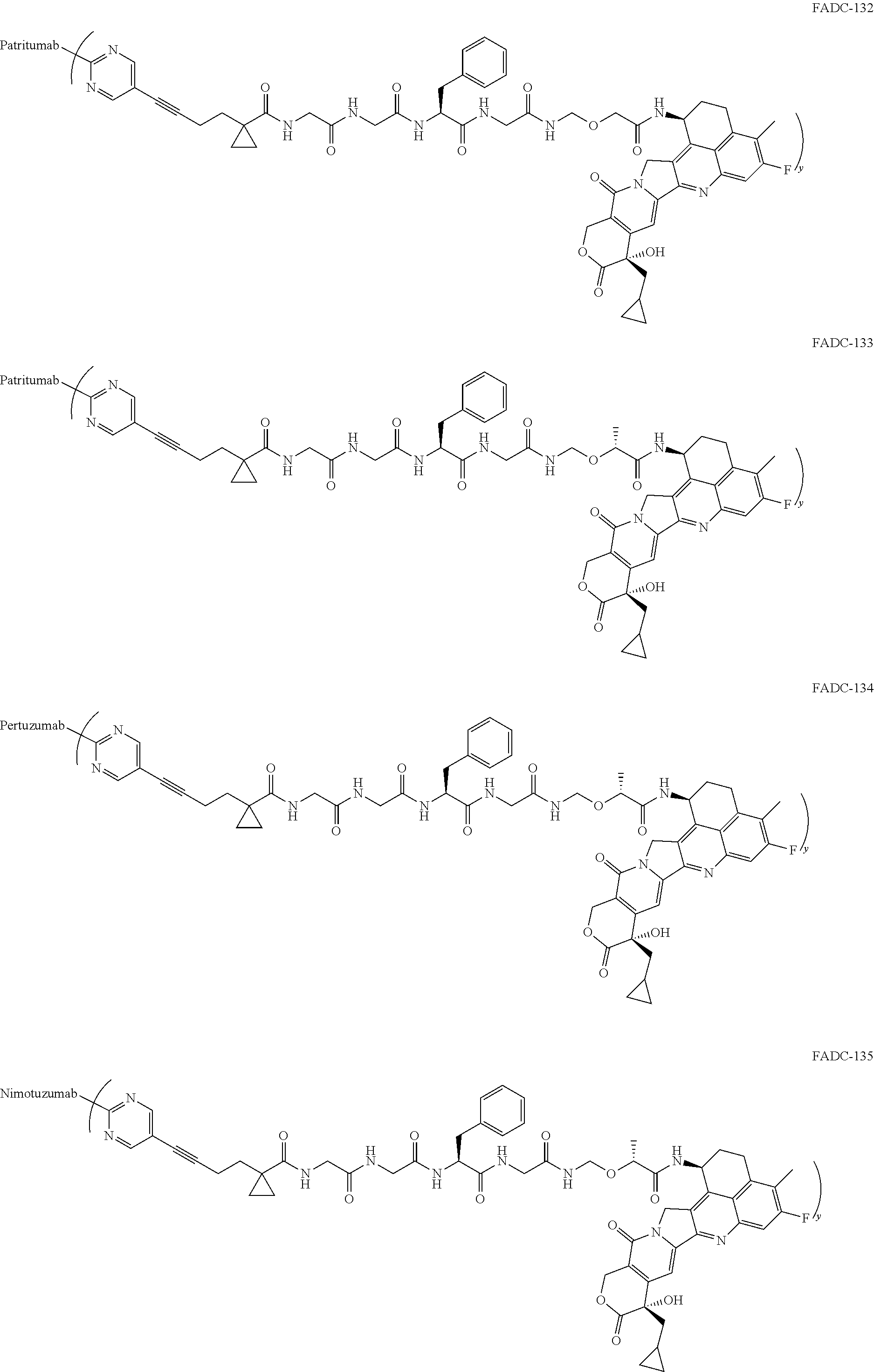

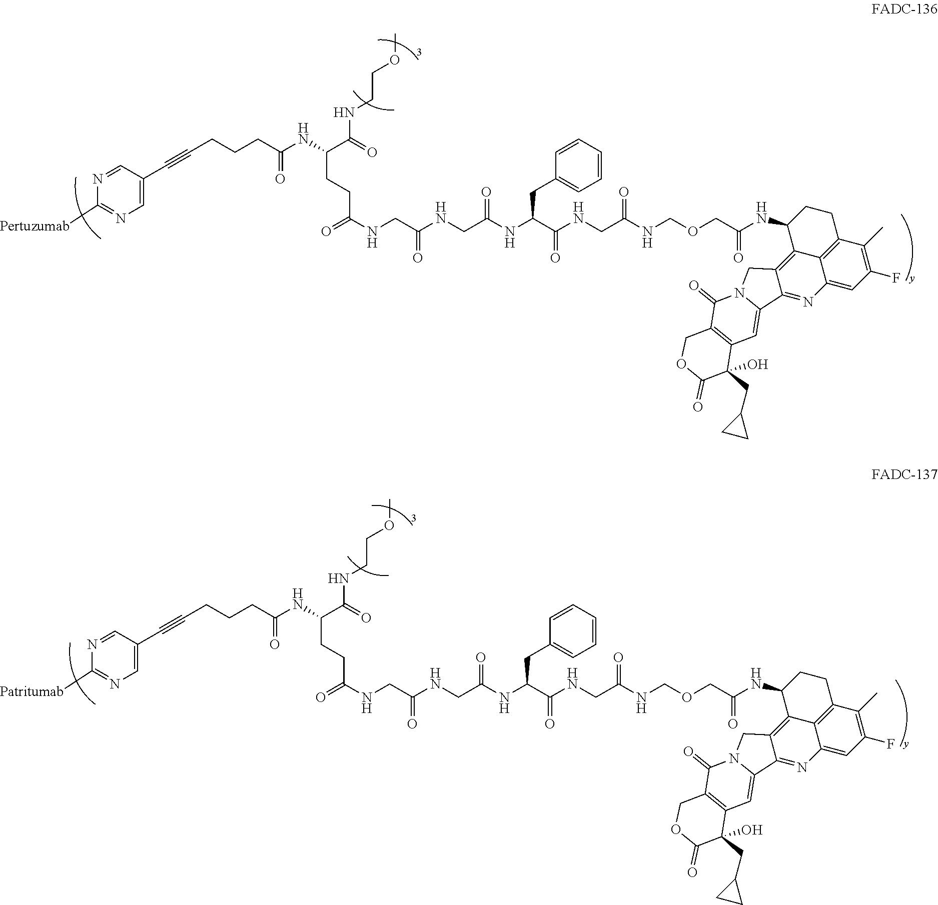

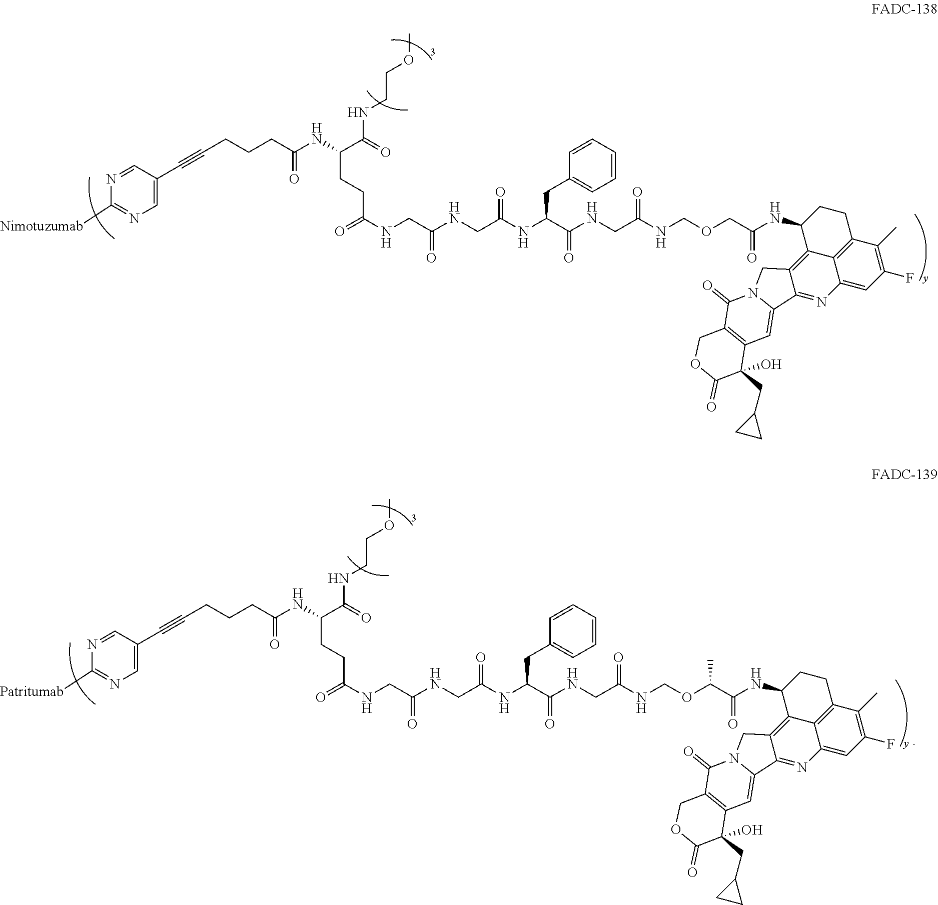

34. A method for preparing the conjugate, or a stereoisomer, racemate, tautomer, isotopologue, isotope-labeled compound, nitroxide or pharmaceutically acceptable salt thereof according to claim 1, comprising:
Step 1: providing a linker as shown in L¹-L²-L³-L⁴-L^{5′} (L′);
preferably, in the linker,
L⁴-L^{5′} is: —NR^{z1}—C(R^{z2}R^{z3})-T^{L}- in its carboxylic acid form or active ester form;
wherein:
R^{z1} is H or C₁₋₄ alkyl;
R^{z2} and R^{z3} are the same or different and are independently selected from H or C₁₋₄ alkyl;
further preferably:
L⁴-L^{5′} is: —NR¹—C(R²R³)—O—(CH₂)ₘ—C(R^{z4}R^{z5})—CO— in its carboxylic acid form or active ester form;
wherein:
R^{z1} is H or C₁₋₄ alkyl;
m is an integer selected from 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
Step 2: coupling the linker with an intermediate compound as represented by Formula (G′) to obtain a coupled intermediate as shown in L^{1′}-L²-L³-L⁴-L⁵-G (C′);
the structure of the intermediate compound as represented by Formula (G′) is: 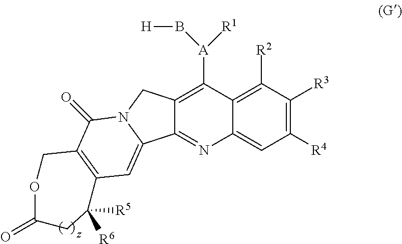
wherein, A, B, T^{L}, R¹, R², R³, R⁴, R⁵, R⁶, L^{1′}, L¹, L², L³, L⁴, L⁵, and L^{5′} independently have the definitions as described in claim 1;
preferably, the preparation method further comprises Step 3: coupling the coupled intermediate as represented by Formula (C′) with the targeting moiety Tp;
optionally, if necessary, functional groups of reaction substrates can also be protected with a protecting group known in the art to allow the reaction to proceed smoothly, and the protecting group is removed after the reaction is completed.

35. A compound represented by the following Formula (GH), or a stereoisomer, racemate, tautomer, isotopologue, isotope-labeled compound, nitroxide or pharmaceutically acceptable salt thereof: 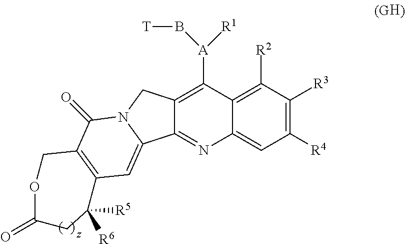
wherein, T is selected from H-T^{L};
T^{L}, A, B, R¹, R², R³, R⁴, R⁵, R⁶, and z independently have the definitions as described in claim 1;
particularly, the compound represented by Formula (GH) is selected from the compound represented by the following Formulas: 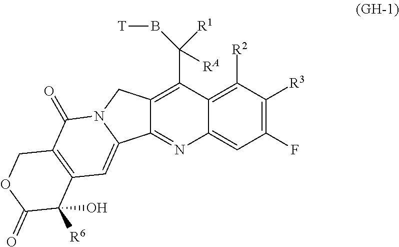
wherein, R^{A}, B, T, R¹, R², R³, and R⁶ independently have the definitions as described in claim 1; 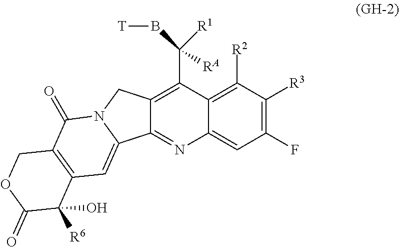
wherein, R^{A}, B, T, R¹, R², R³, and R⁶ independently have the definitions as described in claim 1; 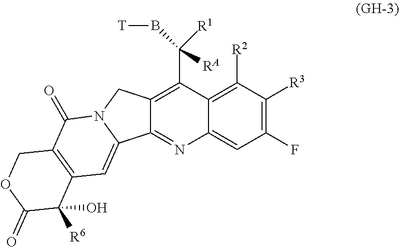
wherein, R^{A}, B, T, R¹, R², R³, and R⁶ independently have the definitions as described in claim 1.

36-37. (canceled)

38. The compound, or a stereoisomer, racemate, tautomer, isotopologue, isotope-labeled compound, nitroxide or pharmaceutically acceptable salt thereof according to claim 35, wherein the compound is selected from the following compounds:

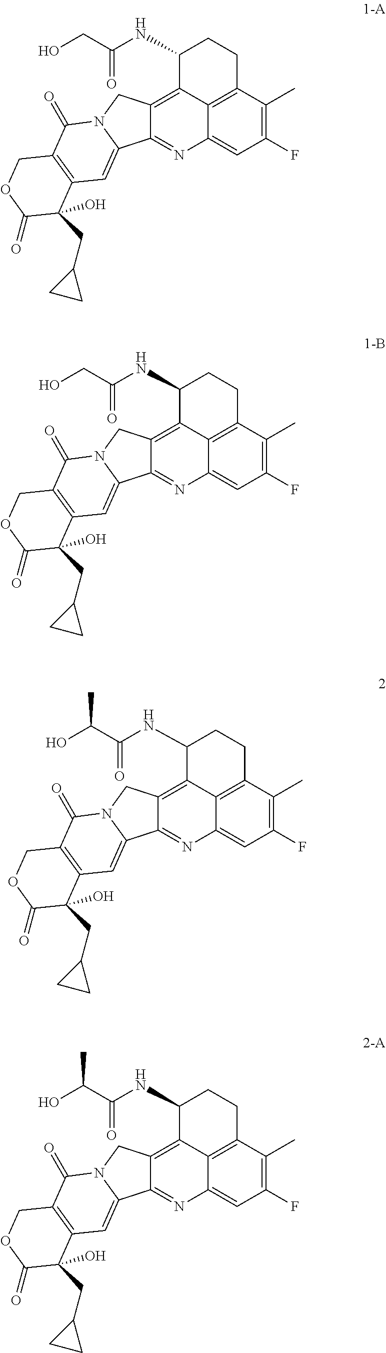 [SMILES: Cc1c(F)cc2nc3c(c4c2c1CC[C@H]4NC(=O)CO)Cn1c-3cc2c(c1=O)COC(=O)[C@]2(O)CC1CC1 ; Cc1c(F)cc2nc3c(c4c2c1CC[C@@H]4NC(=O)CO)Cn1c-3cc2c(c1=O)COC(=O)[C@]2(O)CC1CC1 ; Cc1c(F)cc2nc3c(c4c2c1CCC4NC(=O)[C@H](C)O)Cn1c-3cc2c(c1=O)COC(=O)[C@]2(O)CC1CC1 ; Cc1c(F)cc2nc3c(c4c2c1CC[C@@H]4NC(=O)[C@H](C)O)Cn1c-3cc2c(c1=O)COC(=O)[C@]2(O)CC1CC1]

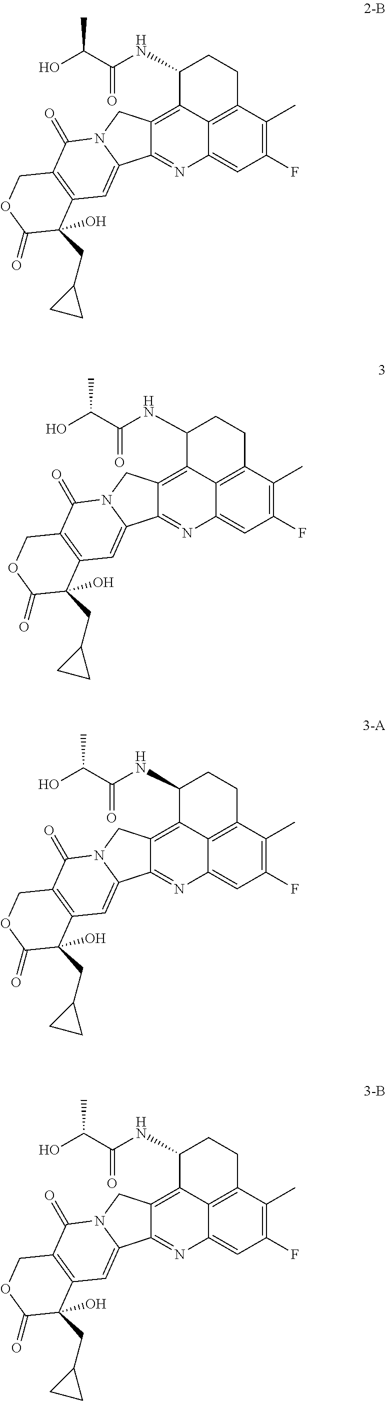 [SMILES: Cc1c(F)cc2nc3c(c4c2c1CC[C@H]4NC(=O)[C@H](C)O)Cn1c-3cc2c(c1=O)COC(=O)[C@]2(O)CC1CC1 ; Cc1c(F)cc2nc3c(c4c2c1CCC4NC(=O)[C@@H](C)O)Cn1c-3cc2c(c1=O)COC(=O)[C@]2(O)CC1CC1 ; Cc1c(F)cc2nc3c(c4c2c1CC[C@@H]4NC(=O)[C@@H](C)O)Cn1c-3cc2c(c1=O)COC(=O)[C@]2(O)CC1CC1 ; Cc1c(F)cc2nc3c(c4c2c1CC[C@H]4NC(=O)[C@@H](C)O)Cn1c-3cc2c(c1=O)COC(=O)[C@]2(O)CC1CC1]

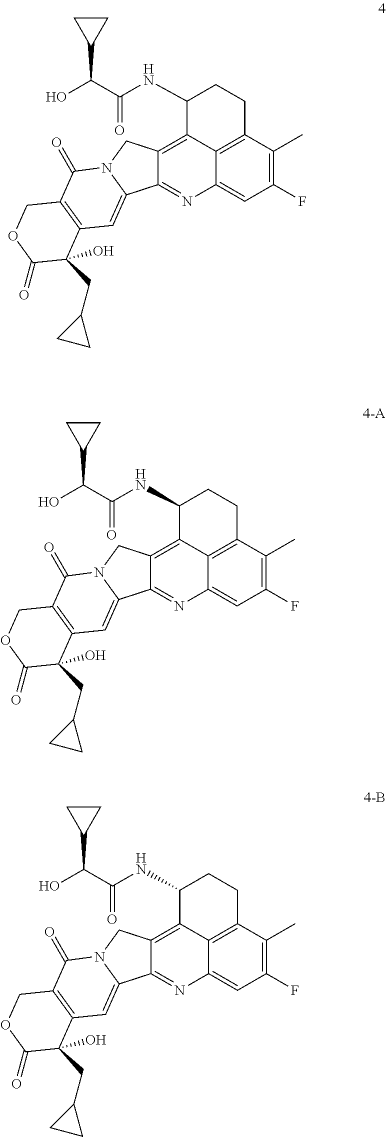 [SMILES: Cc1c(F)cc2nc3c(c4c2c1CCC4NC(=O)[C@@H](O)C1CC1)Cn1c-3cc2c(c1=O)COC(=O)[C@]2(O)CC1CC1 ; Cc1c(F)cc2nc3c(c4c2c1CC[C@@H]4NC(=O)[C@@H](O)C1CC1)Cn1c-3cc2c(c1=O)COC(=O)[C@]2(O)CC1CC1 ; Cc1c(F)cc2nc3c(c4c2c1CC[C@H]4NC(=O)[C@@H](O)C1CC1)Cn1c-3cc2c(c1=O)COC(=O)[C@]2(O)CC1CC1]

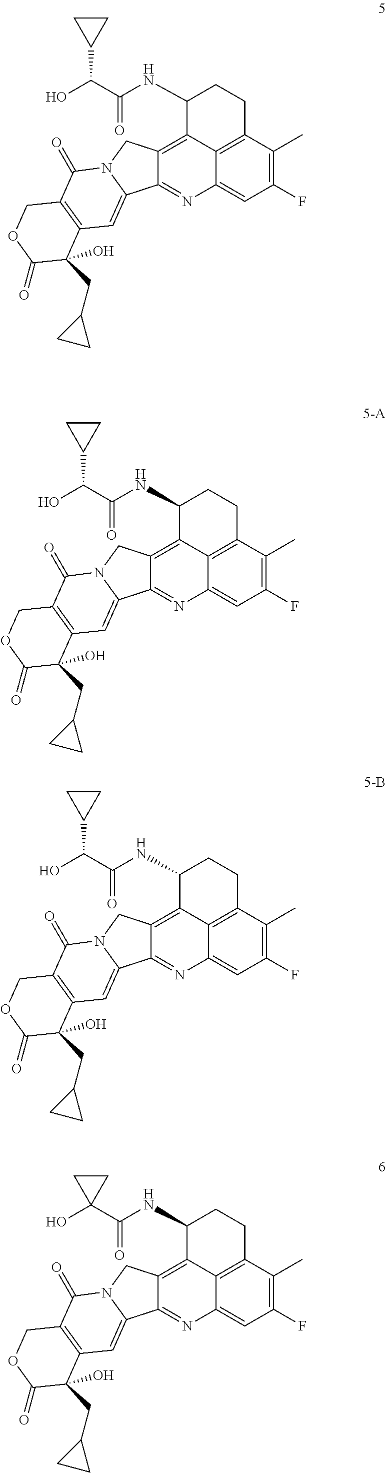

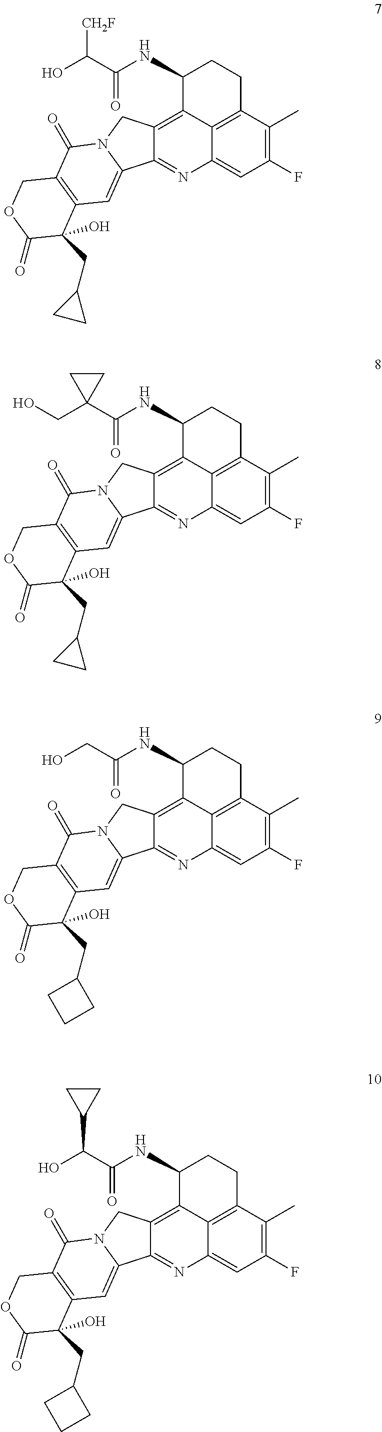

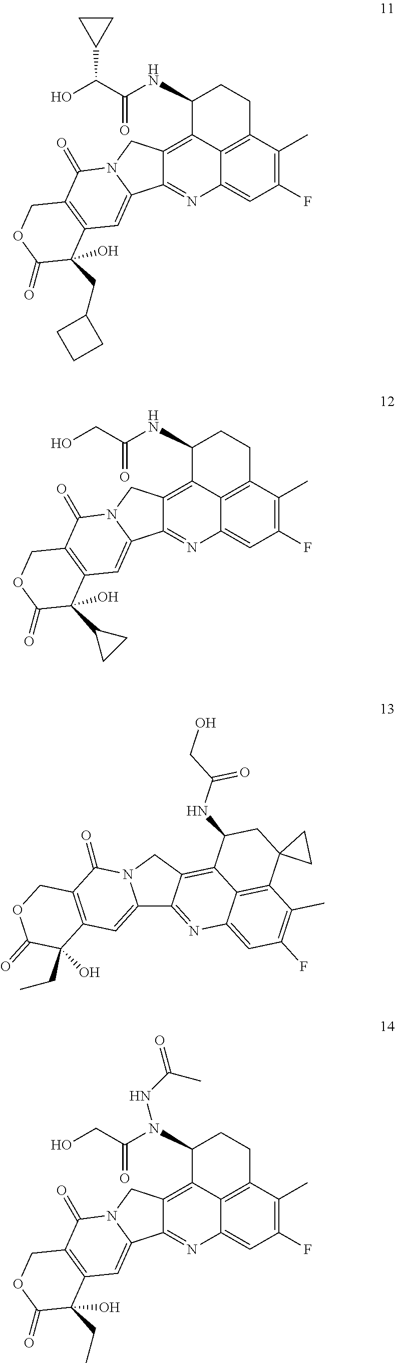 [SMILES: Cc1c(F)cc2nc3c(c4c2c1CC[C@@H]4NC(=O)[C@H](O)C1CC1)Cn1c-3cc2c(c1=O)COC(=O)[C@]2(O)CC1CCC1 ; Cc1c(F)cc2nc3c(c4c2c1CC[C@@H]4NC(=O)CO)Cn1c-3cc2c(c1=O)COC(=O)[C@]2(O)C1CC1 ; CC[C@@]1(O)C(=O)OCc2c1cc1n(c2=O)Cc2c-1nc1cc(F)c(C)c3c1c2[C@@H](NC(=O)CO)CC31CC1 ; CC[C@@]1(O)C(=O)OCc2c1cc1n(c2=O)Cc2c-1nc1cc(F)c(C)c3c1c2[C@@H](N(NC(C)=O)C(=O)CO)CC3]

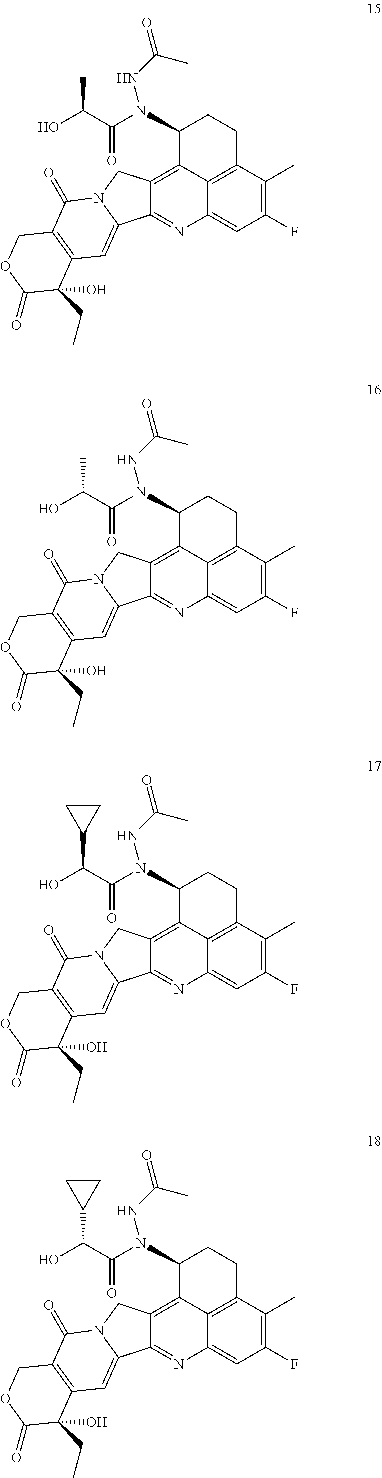 [SMILES: CC[C@@]1(O)C(=O)OCc2c1cc1n(c2=O)Cc2c-1nc1cc(F)c(C)c3c1c2[C@@H](N(NC(C)=O)C(=O)[C@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](C)O)CC3 ; CC[C@@]1(O)C(=O)OCc2c1cc1n(c2=O)Cc2c-1nc1cc(F)c(C)c3c1c2[C@@H](N(NC(C)=O)C(=O)[C@@H](O)C1CC1)CC3 ; CC[C@@]1(O)C(=O)OCc2c1cc1n(c2=O)Cc2c-1nc1cc(F)c(C)c3c1c2[C@@H](N(NC(C)=O)C(=O)[C@H](O)C1CC1)CC3]

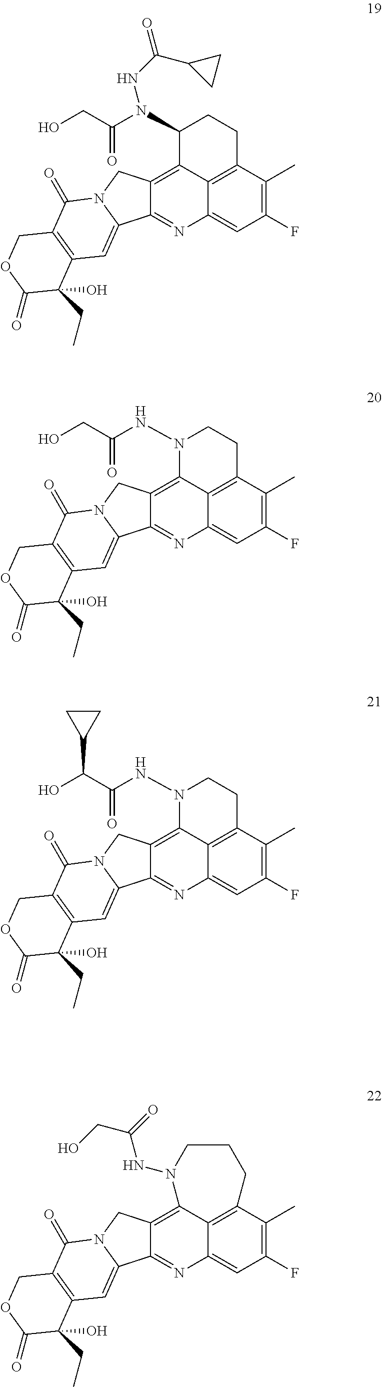 [SMILES: CC[C@@]1(O)C(=O)OCc2c1cc1n(c2=O)Cc2c-1nc1cc(F)c(C)c3c1c2[C@@H](N(NC(=O)C1CC1)C(=O)CO)CC3 ; CC[C@@]1(O)C(=O)OCc2c1cc1n(c2=O)Cc2c-1nc1cc(F)c(C)c3c1c2N(NC(=O)CO)CC3 ; CC[C@@]1(O)C(=O)OCc2c1cc1n(c2=O)Cc2c-1nc1cc(F)c(C)c3c1c2N(NC(=O)[C@@H](O)C1CC1)CC3 ; CC[C@@]1(O)C(=O)OCc2c1cc1n(c2=O)Cc2c-1nc1cc(F)c(C)c3c1c2N(NC(=O)CO)CCC3]

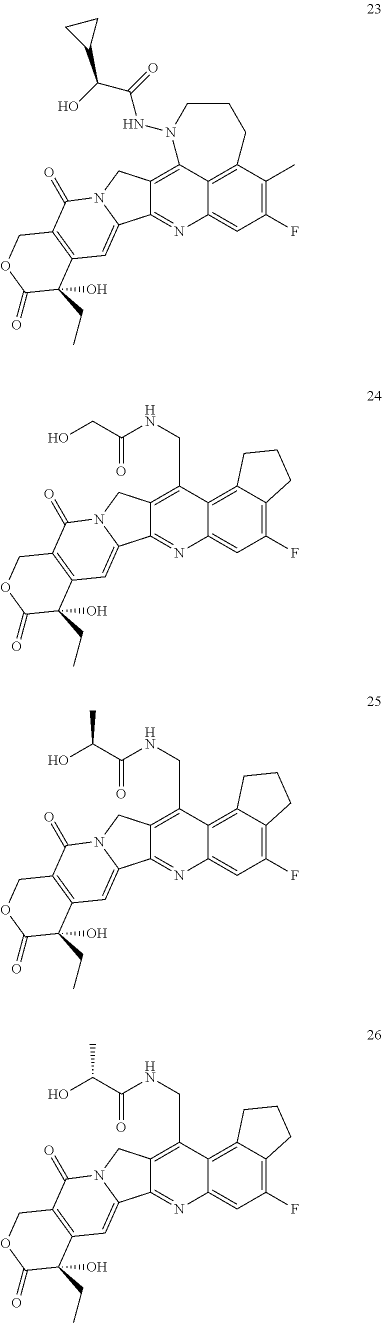 [SMILES: CC[C@@]1(O)C(=O)OCc2c1cc1n(c2=O)Cc2c-1nc1cc(F)c(C)c3c1c2N(NC(=O)[C@@H](O)C1CC1)CCC3 ; CC[C@@]1(O)C(=O)OCc2c1cc1n(c2=O)Cc2c-1nc1cc(F)c3c(c1c2CNC(=O)CO)CCC3 ; CC[C@@]1(O)C(=O)OCc2c1cc1n(c2=O)Cc2c-1nc1cc(F)c3c(c1c2CNC(=O)[C@H](C)O)CCC3 ; CC[C@@]1(O)C(=O)OCc2c1cc1n(c2=O)Cc2c-1nc1cc(F)c3c(c1c2CNC(=O)[C@@H](C)O)CCC3]

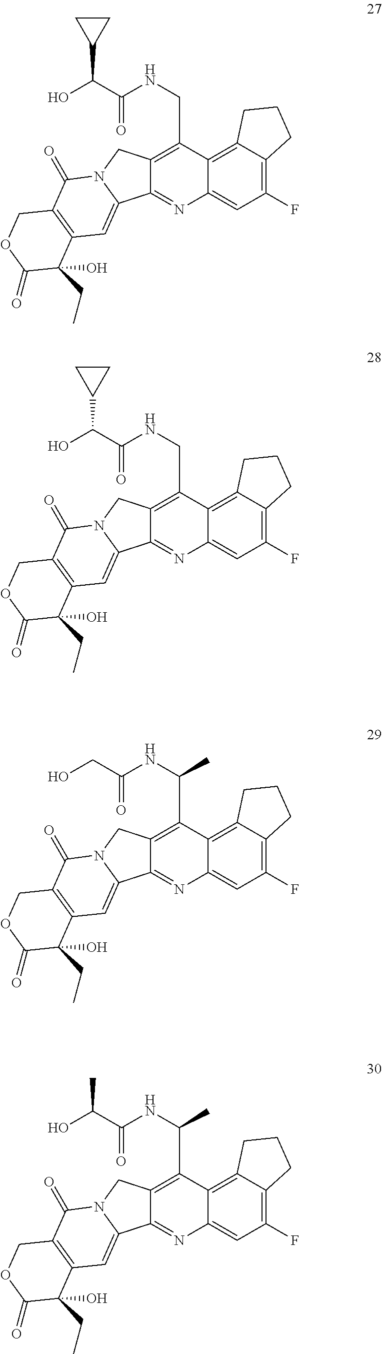

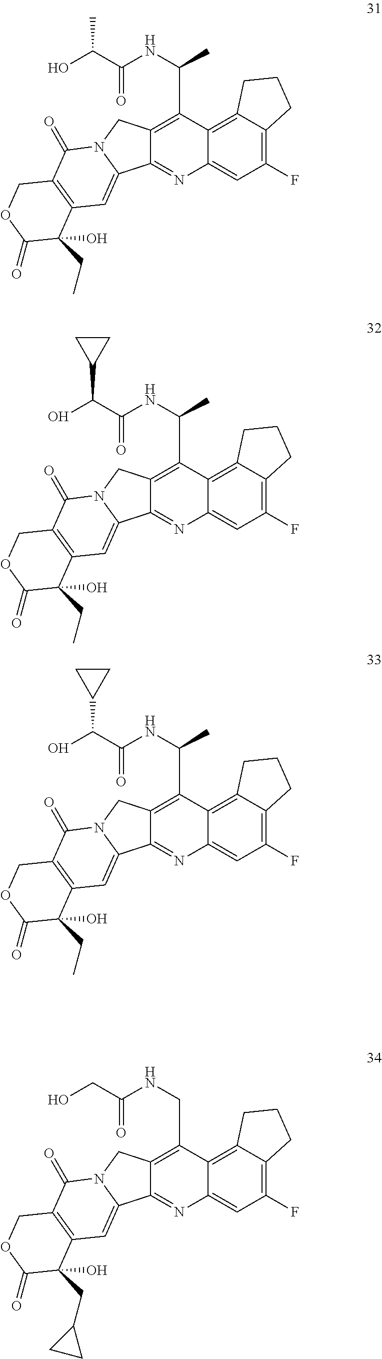

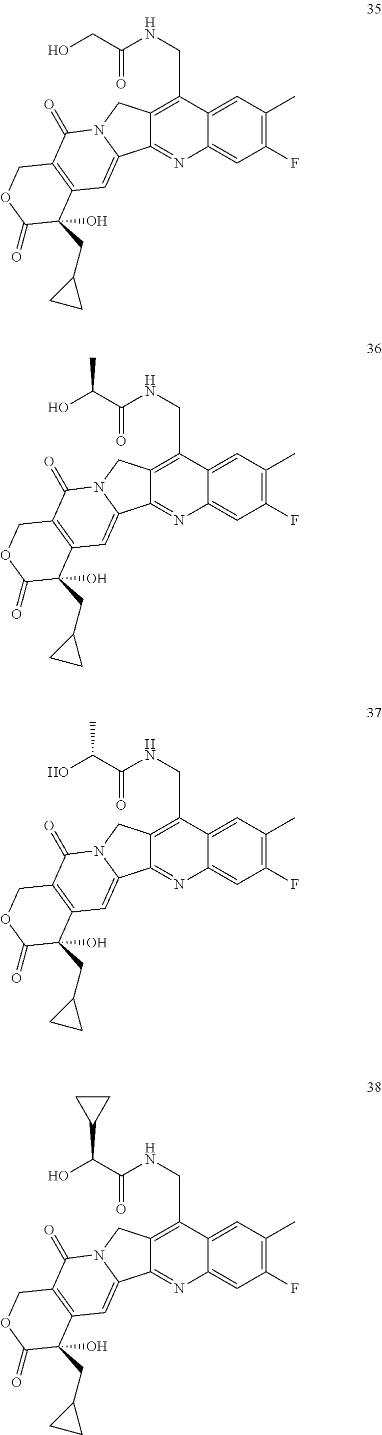 [SMILES: Cc1cc2c(CNC(=O)CO)c3c(nc2cc1F)-c1cc2c(c(=O)n1C3)COC(=O)[C@]2(O)CC1CC1 ; Cc1cc2c(CNC(=O)[C@H](C)O)c3c(nc2cc1F)-c1cc2c(c(=O)n1C3)COC(=O)[C@]2(O)CC1CC1 ; Cc1cc2c(CNC(=O)[C@@H](C)O)c3c(nc2cc1F)-c1cc2c(c(=O)n1C3)COC(=O)[C@]2(O)CC1CC1 ; Cc1cc2c(CNC(=O)[C@@H](O)C3CC3)c3c(nc2cc1F)-c1cc2c(c(=O)n1C3)COC(=O)[C@]2(O)CC1CC1]

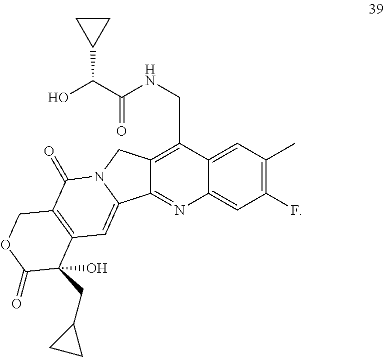 [SMILES: Cc1cc2c(CNC(=O)[C@H](O)C3CC3)c3c(nc2cc1F)-c1cc2c(c(=O)n1C3)COC(=O)[C@]2(O)CC1CC1]

39. A method for preparing the compound, or a stereoisomer, racemate, tautomer, isotopologue, isotope-labeled compound, nitroxide or pharmaceutically acceptable salt thereof according to claim 35, the method comprises a step of reacting a compound represented by Formula (i) with a compound represented by Formula (ii): 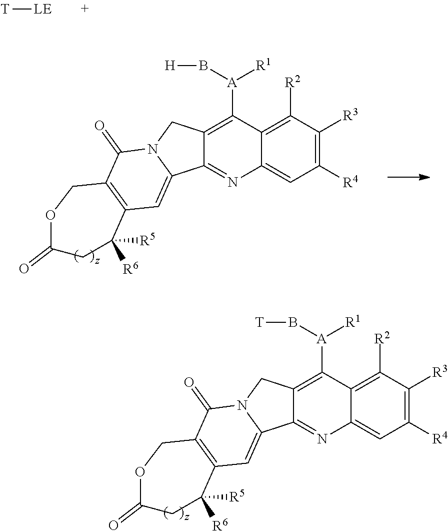
wherein, A, B, T, R¹, R², R³, R⁴, R⁵, R⁶, and z independently have the definitions as described in claim 35;
LE represents a group that is removed or leaves after the reaction.

40. A method for preparing the compound represented by Formula (ii) in claim 35, the method comprises a step of reacting a compound represented by Formula (iii) with a compound represented by Formula (iv) to obtain a compound represented by Formula (v): 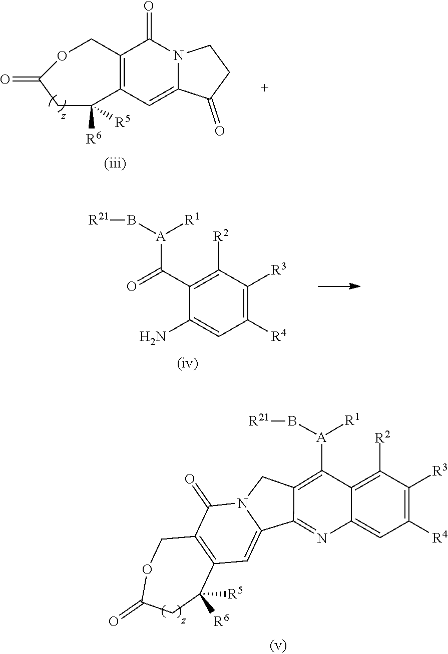
wherein, A, B, R¹, R², R³, R⁴, R⁵, R⁶, and z independently have the definitions as described in claim 35;
R²¹ is selected from H or a protecting group, preferably an amino protecting group.

41. A compound represented by Formula (iii): 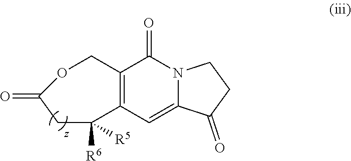
wherein, R⁵, R⁶, and z independently have the definitions as described in claim 35.

42. A compound represented by Formula (v): 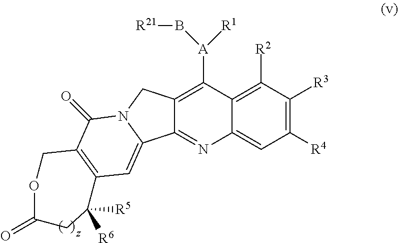
wherein, A, B, R¹, R², R³, R⁴, R⁵, R⁶, R²¹, and z independently have the definitions as described in claim 35.

43. A conjugate of the following structure: 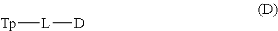
wherein, Tp represents a targeting moiety;
D represents a bioactive molecular fragment, preferably a molecular fragment with anti-tumor biological activity;
wherein, L is selected from the linker represented by Formula (L): 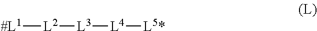
wherein, L¹ is a linking moiety to the targeting moiety Tp, and formed from a reactive group L^{1′} through its reaction with the targeting moiety Tp, and # represents the site connected to the Tp moiety;
L² is absent or a spacer between L¹ and L³;
L³ is a peptide moiety;
L⁴ is absent or a spacer between the peptide moiety and L⁵;
L⁵ is a linking moiety connecting L⁴ with the bioactive molecule D, and formed from a reactive group L^{5′} through its reaction with the bioactive molecule, and * represents the site connected to the bioactive molecule D;
preferably, L^{1′}, L¹, L², L³, L⁴, L⁵, and L^{5′} have the definitions as described in claim 14;
provided that:
Condition I:
the following hydrophilic moiety is inserted between the fragments of L as defined above:
hydrophilic moiety: 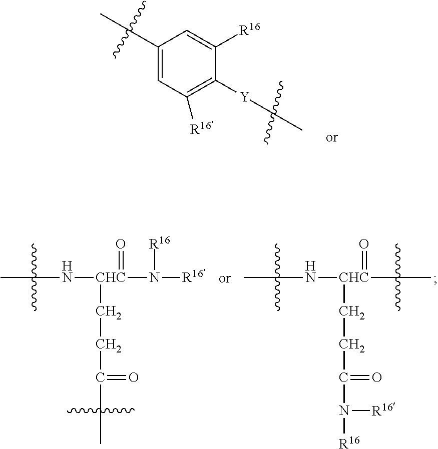
or Condition II:
L¹ is generated by coupling Tp with a sulfhydryl-reactive group contained in maleimide group or a substituted maleimide group;
the following steric hindrance moiety is inserted between L¹ and L², or between L² and L³, or is inserted by replacing L²:
steric hindrance moiety: 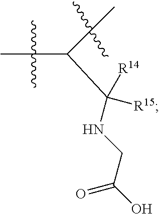
or Condition III:
L¹ is generated by coupling Tp with a sulfhydryl-reactive group contained in Hal-Het-;
L⁴-L⁵ is: —NR^{z1}—C(R^{z2}R^{z3})-T^{L}-;
wherein:
R¹ is H or C₁₋₄ alkyl;
R^{z2} and R^{z3} are the same or different, and are independently selected from H or C₁₋₄ alkyl;
T^{L} has the definition as described in claim 14;
in a further embodiment,
L¹ is generated by coupling Tp with a sulfhydryl-reactive group contained in maleimide group or Hal-Het-;
the following hydrophilic moiety is inserted between L¹ and L², or between L² and L³, or is inserted by replacing L²:
hydrophilic moiety: 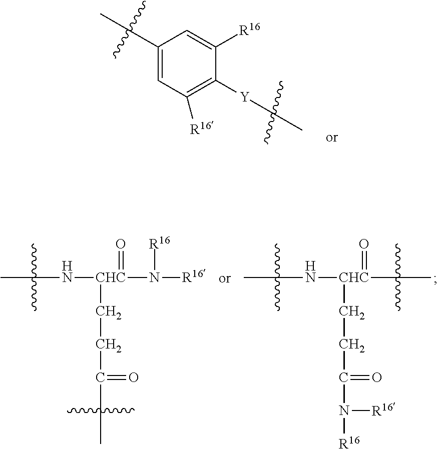
in a further embodiment:
L¹ is generated by coupling Tp with a sulfhydryl-reactive group contained in Hal-Het-;
L⁴-L⁵ is: —NR^{z1}—C(R^{z2}R^{z3})—O—(CH₂)ₘ—C(R^{z4}R^{z5})—CO—;
wherein:
R^{z1} is H or C₁₋₄ alkyl;
m is an integer of 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
or Condition IV:
L¹-L² is generated by coupling Tp with L^{1′}-L² of the following structure: 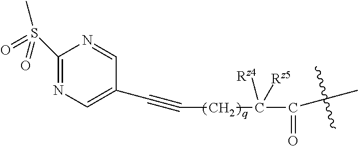
q is an integer selected from 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
it is further preferred that R^{z4} and R^{z5} together form a C₃₋₆ cycloalkyl;
L¹-L² is most preferably generated by coupling Tp with 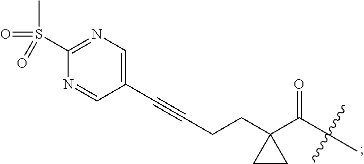 [SMILES: *C(=O)C1(CCC#Cc2cnc(S(C)(=O)=O)nc2)CC1]
preferably, the bioactive molecule D is a compound with biological activity or potential biological activity disclosed in the Chinese Pharmacopoeia, The United States Pharmacopoeia or European Pharmacopoeia or disclosed in other publications;
the drug can be selected from the group consisting of cytotoxic drugs, cytostatic drugs or immunosuppressive drugs, preferably anti-tubulin agents, tubulin inhibitors, DNA minor groove binders, DNA replication inhibitors, alkylating agents, antibiotics, antifolates, antimetabolites, chemosensitizers, topoisomerase inhibitors, vinca alkaloids, etc.;
the drug is further preferably selected from the group consisting of auristatin, camptothecin, duocarmycin, etoposide, maytansine and maytansine alkaloids, taxanes, benzodiazepines or benzodiazepines-containing drugs, and vinca alkaloids.

44. (canceled)

45. A pharmaceutical composition, wherein the pharmaceutical composition comprises the conjugate represented by Formula (C), or a stereoisomer, racemate, tautomer, isotopologue, isotope-labeled compound, nitroxide or pharmaceutically acceptable salt thereof according to claim 1;
preferably, the conjugate represented by the Formula (C), or a stereoisomer, racemate, tautomer, isotopologue, isotope-labeled compound, nitroxide or pharmaceutically acceptable salt thereof in the pharmaceutical composition is present in a therapeutically effective amount.

46. A method for preventing and/or treating a disease or disorder, the method comprising administering to a patient therapeutically effective amount of the compound represented by Formula (GH), or a stereoisomer, racemate, tautomer, isotopologue, isotope-labeled compound, nitroxide or pharmaceutically acceptable salt thereof according claim 35;
preferably, the disease or disorder can be selected from a tumor, such as a solid tumor or a hematological cancer;
preferably, the solid tumor is selected from malignancies of various organ systems, such as sarcomas, adenocarcinomas, blastomas, and carcinomas, such as those affecting liver, lung, breast, lymph, biliary-intestinal (e.g., colon), genitourinary tract (e.g., kidney, urothelial cell), prostate and pharynx. adenocarcinomas comprise, for example, malignancies of most colon cancers, rectal cancers, renal cell carcinomas, liver cancers, small cell lung cancers, non-small cell lung cancers, small intestine cancers and esophageal cancers;
the hematological cancer is selected from the group consisting of leukemia, lymphoma, and malignant lymphoproliferative disorders affecting blood, bone marrow and lymphatic system.

47. (canceled)

48. A conjugate of a linker and a drug, which has the following structure: 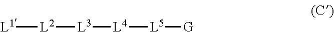
wherein:
G, L^{1′}, L², L³, L⁴, and L⁵ have the definitions as described in claim 14;
particularly, the conjugate is one selected from the following:

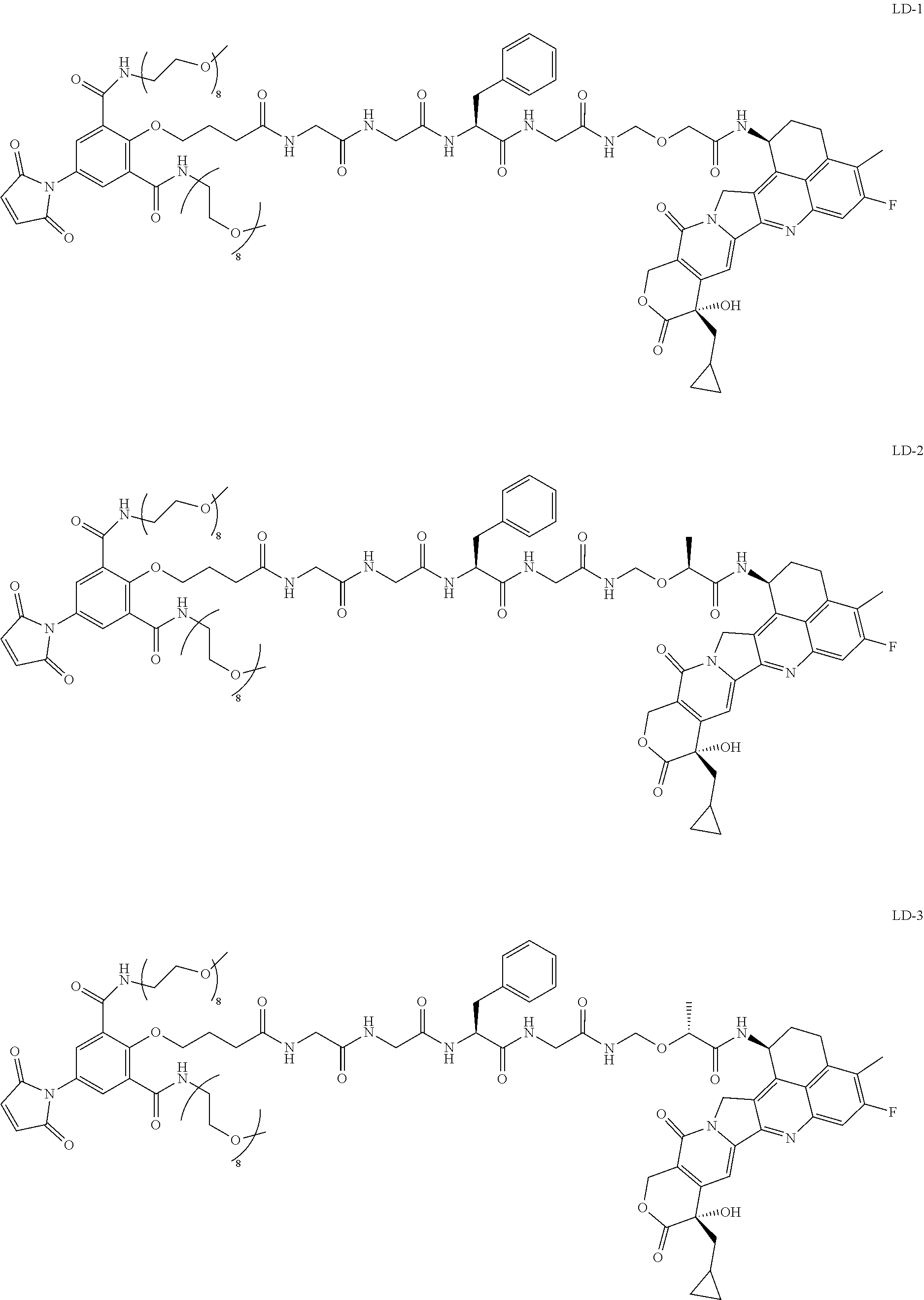

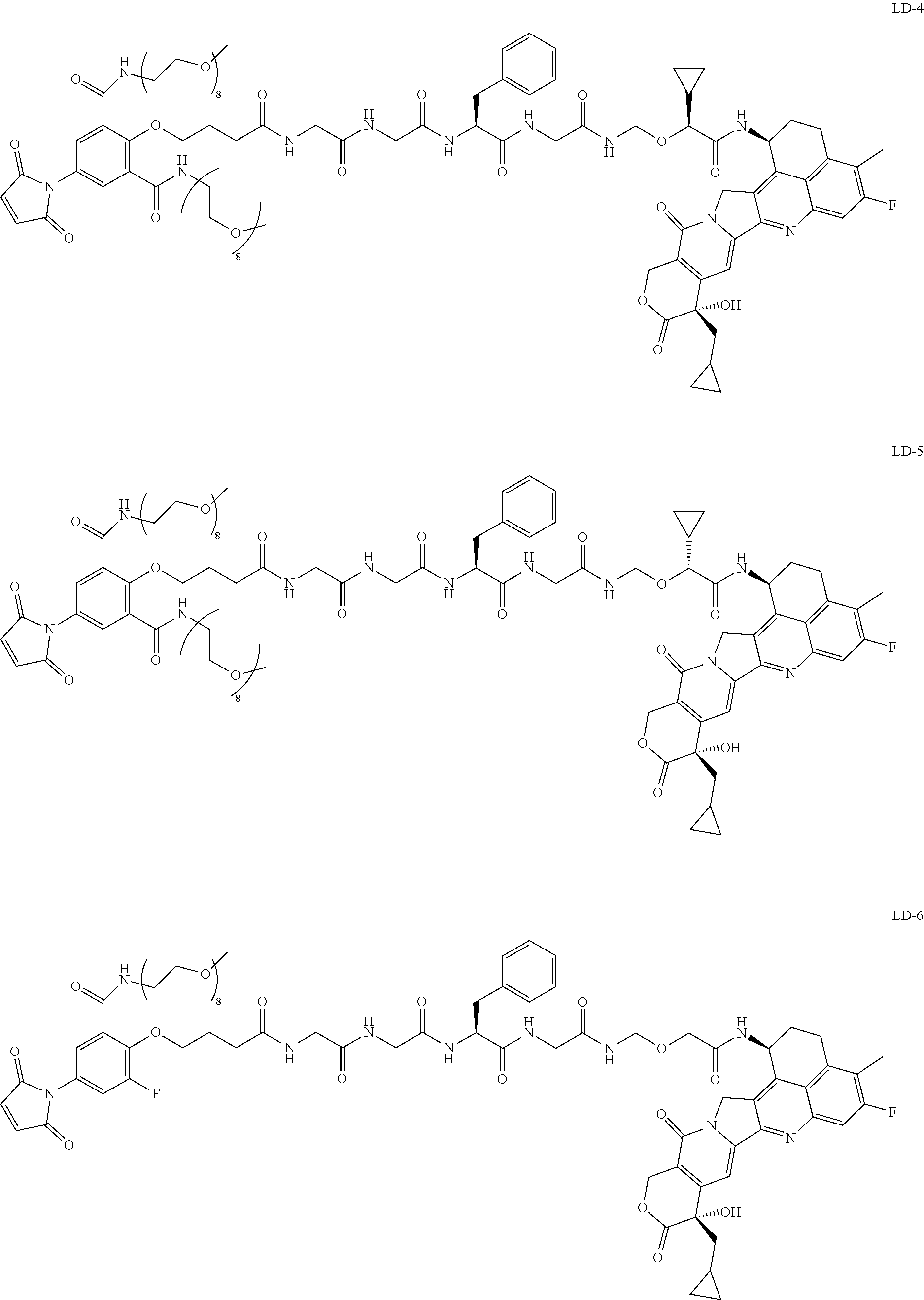

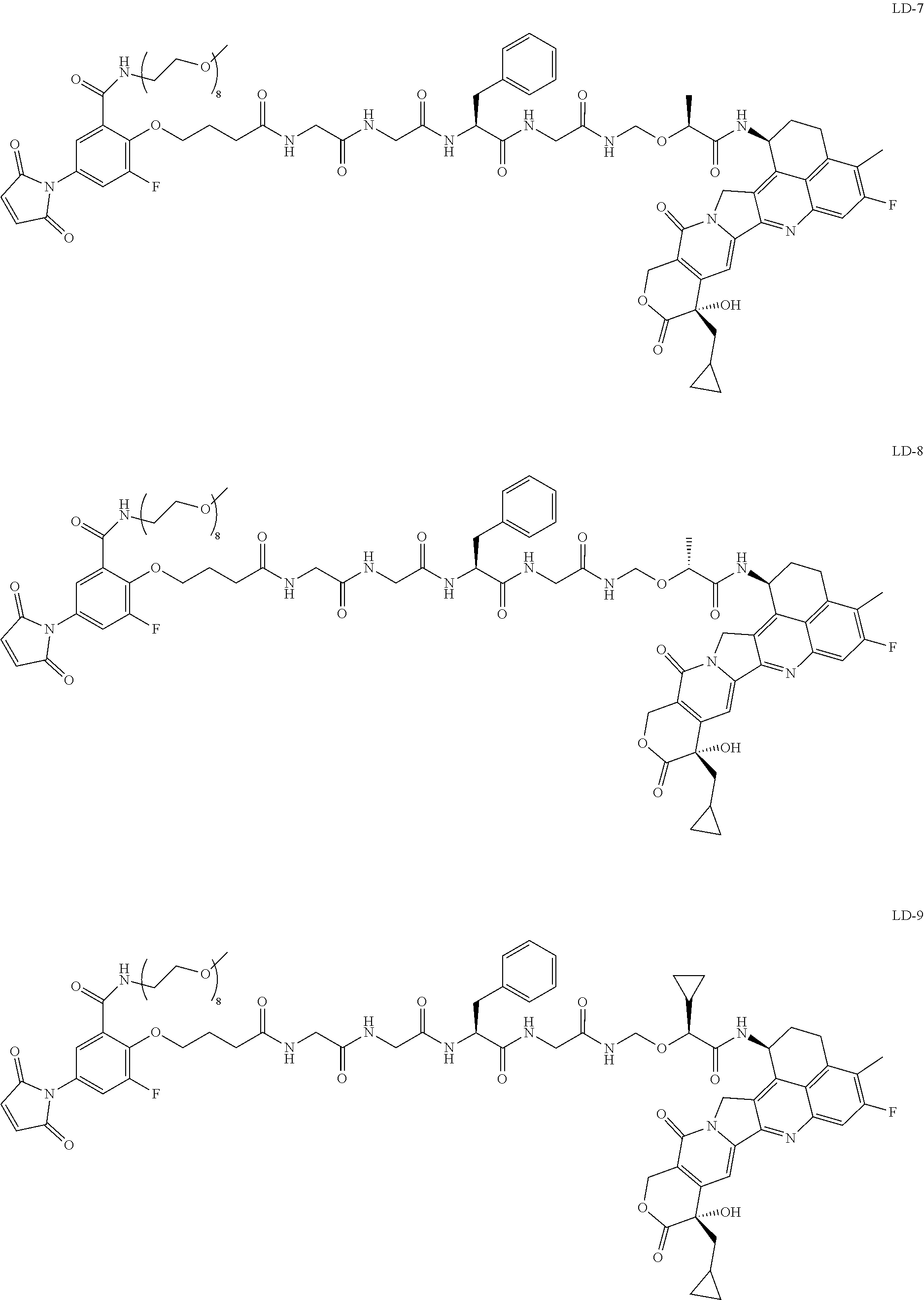

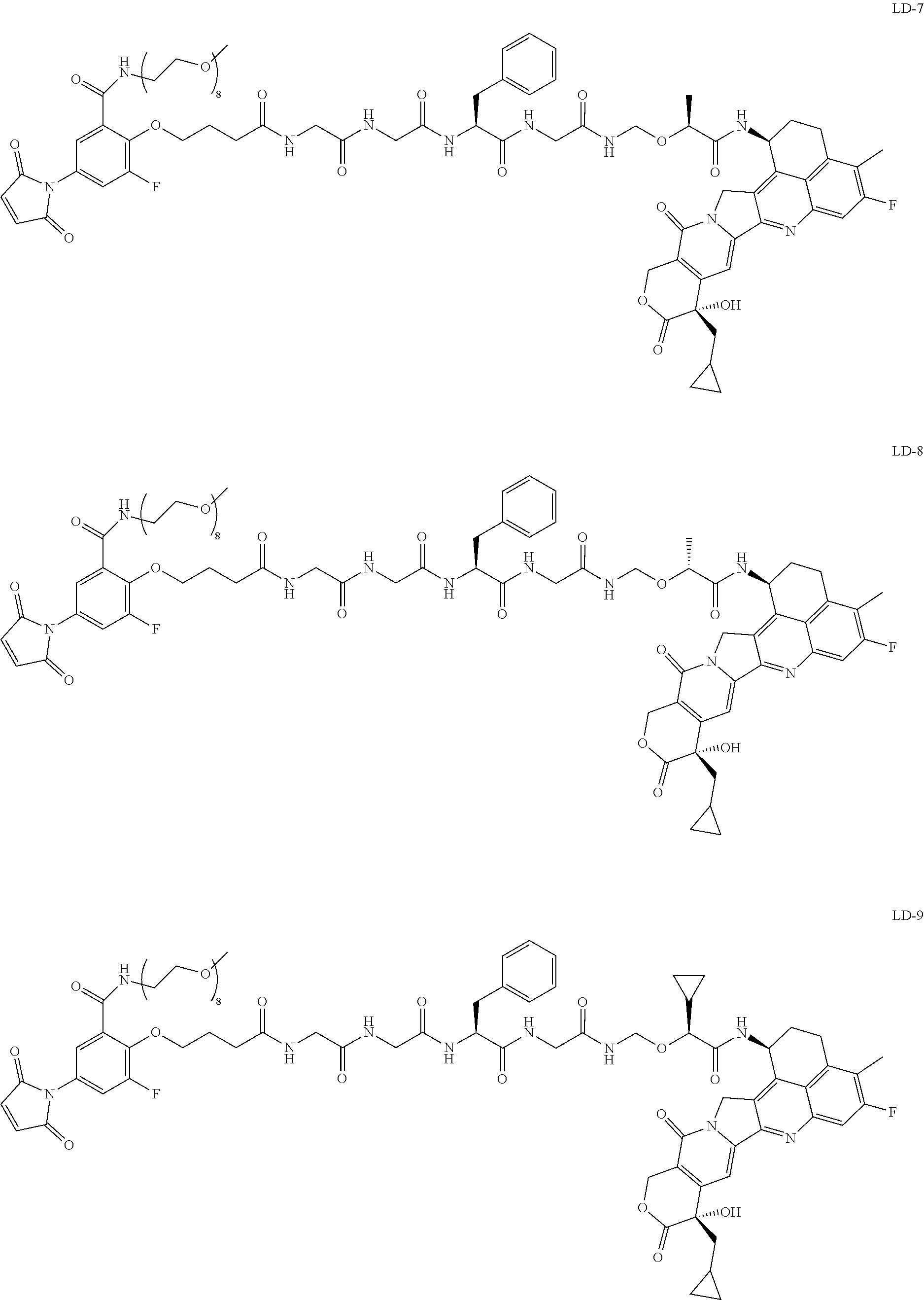

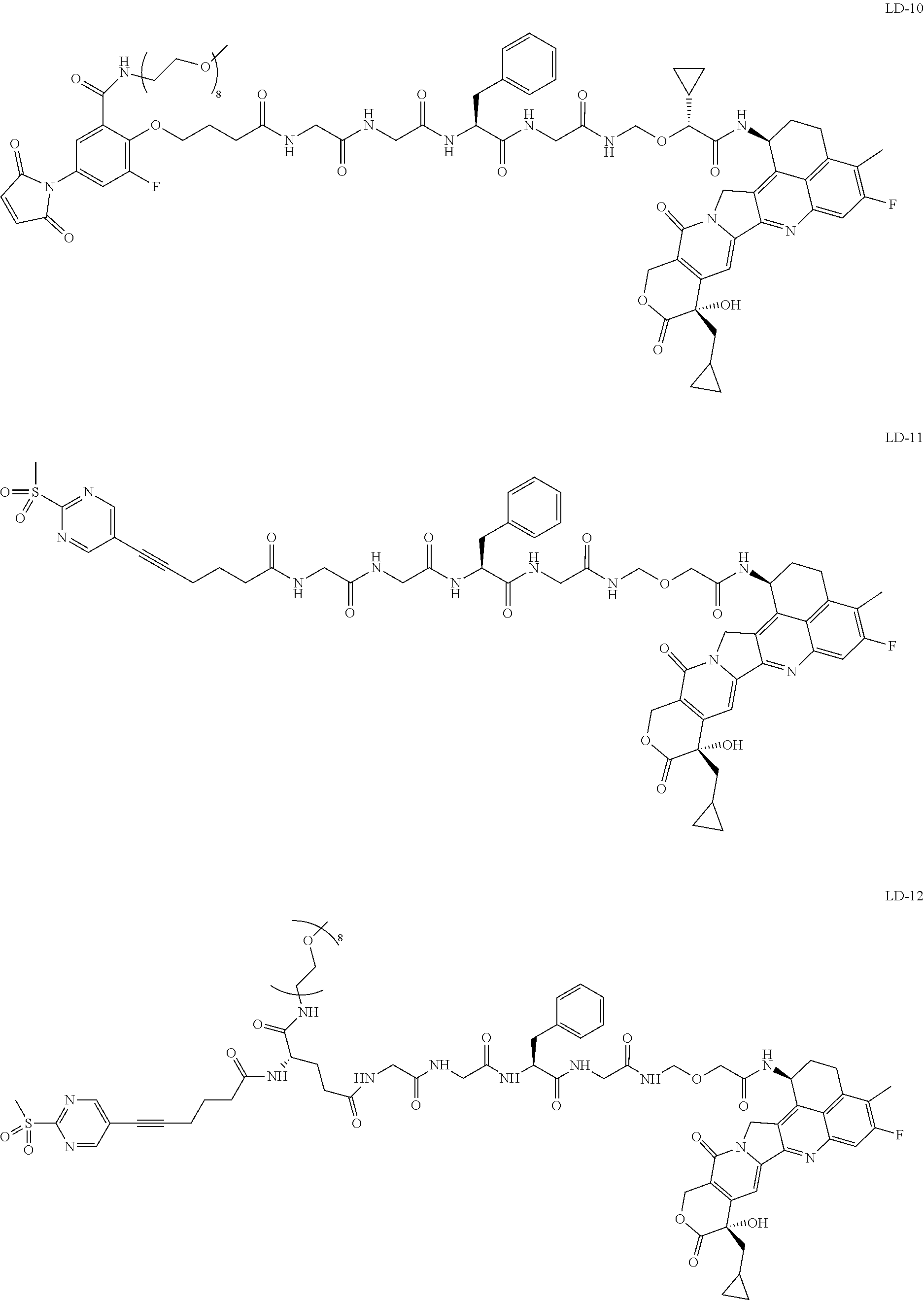

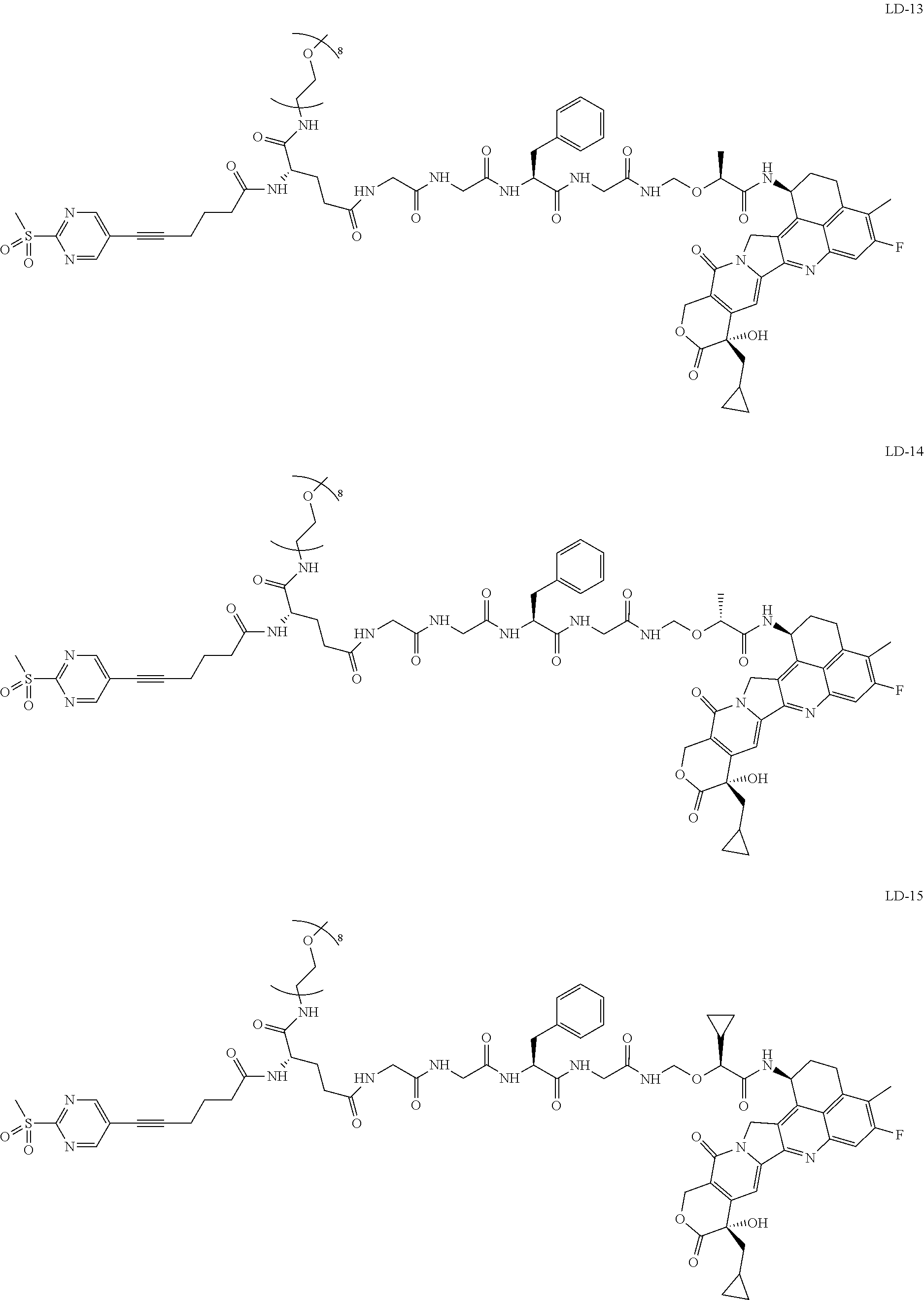

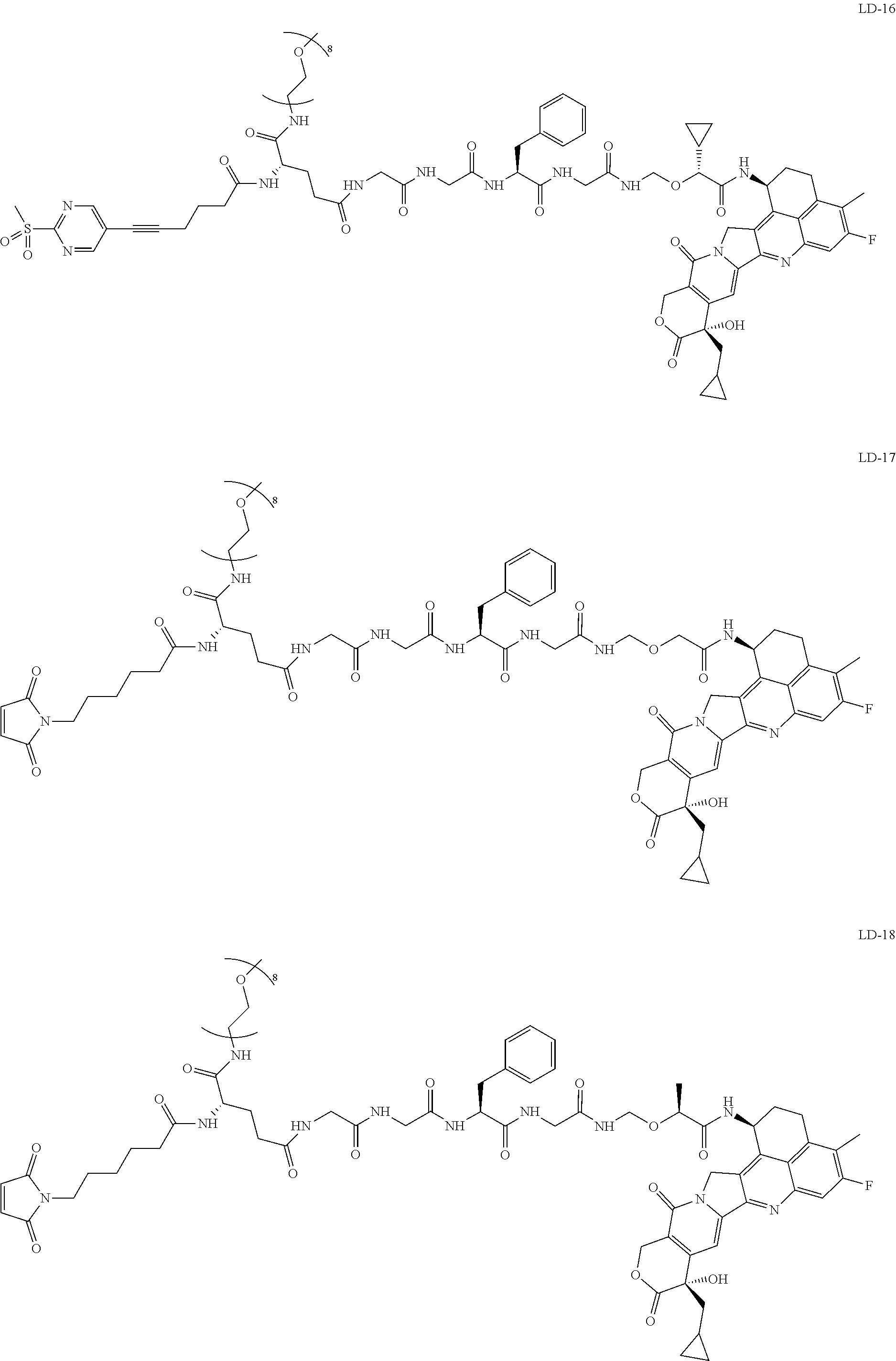

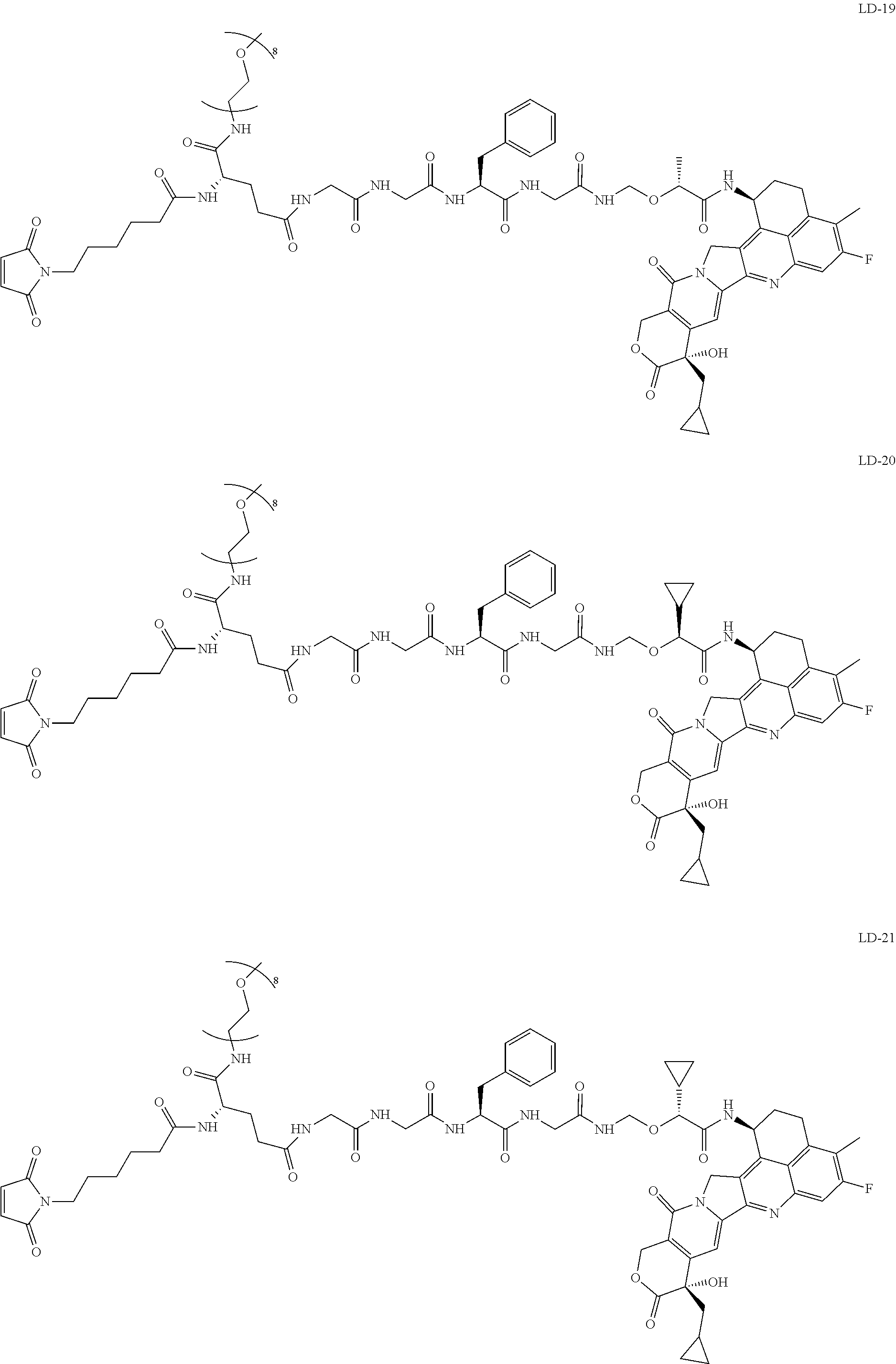

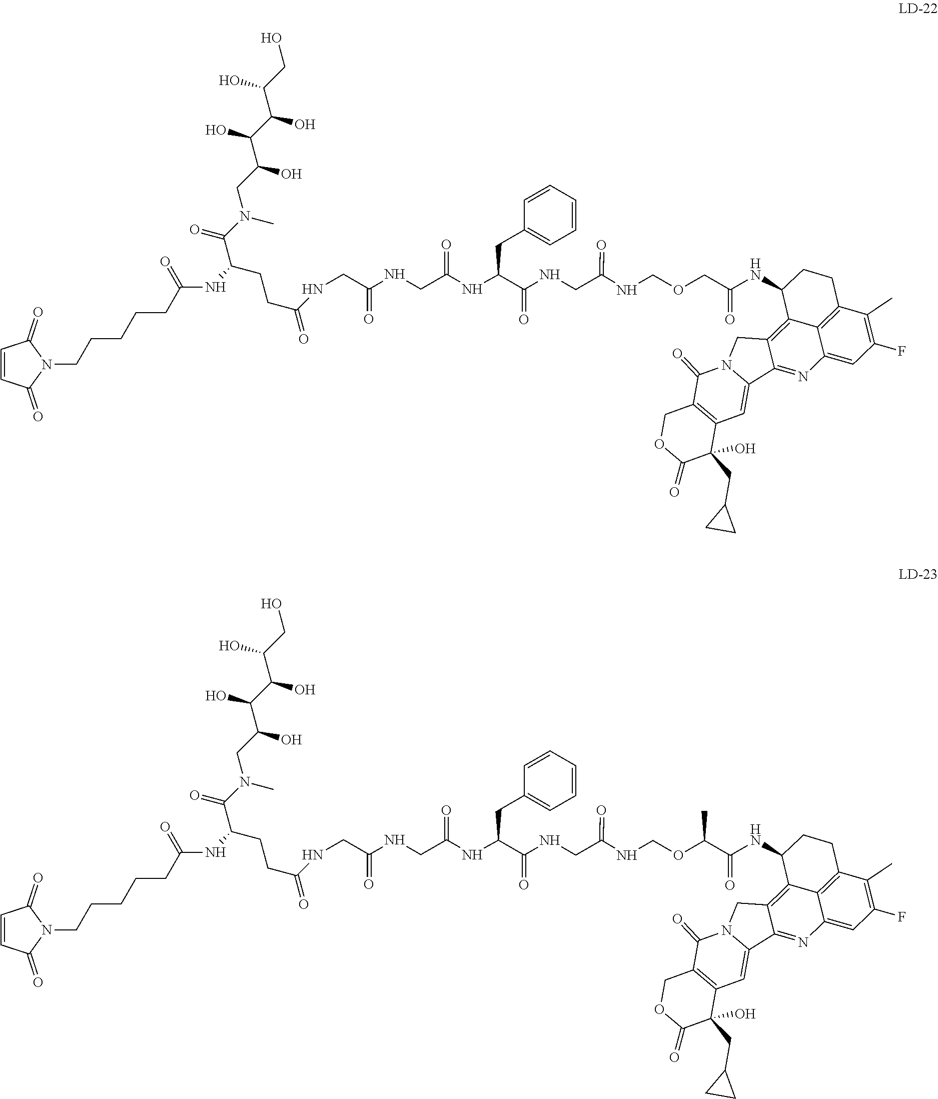 [SMILES: Cc1c(F)cc2nc3c(c4c2c1CC[C@@H]4NC(=O)COCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1]

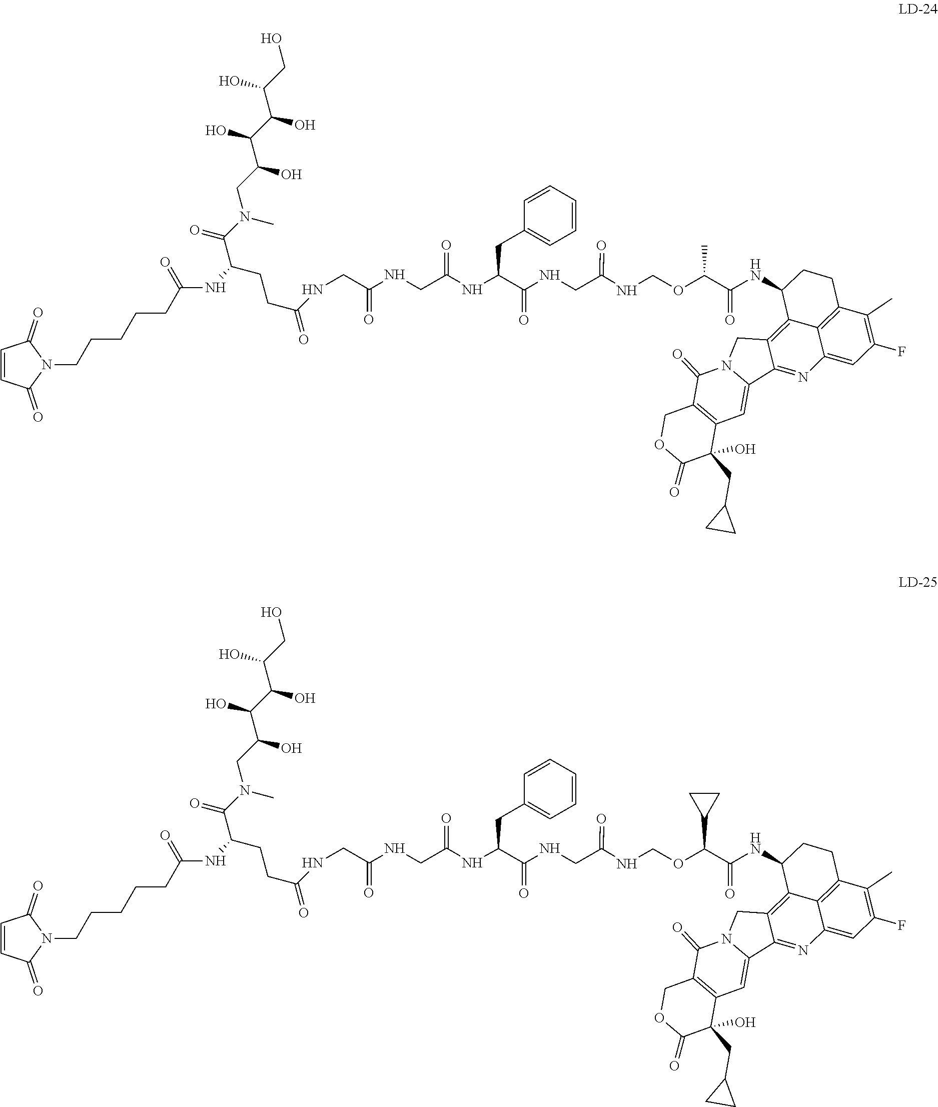 [SMILES: Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)Cn1c-3cc2c(c1=O)COC(=O)[C@]2(O)CC1CC1 ; Cc1c(F)cc2nc3c(c4c2c1CC[C@@H]4NC(=O)[C@@H](OCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)C1CC1)Cn1c-3cc2c(c1=O)COC(=O)[C@]2(O)CC1CC1]

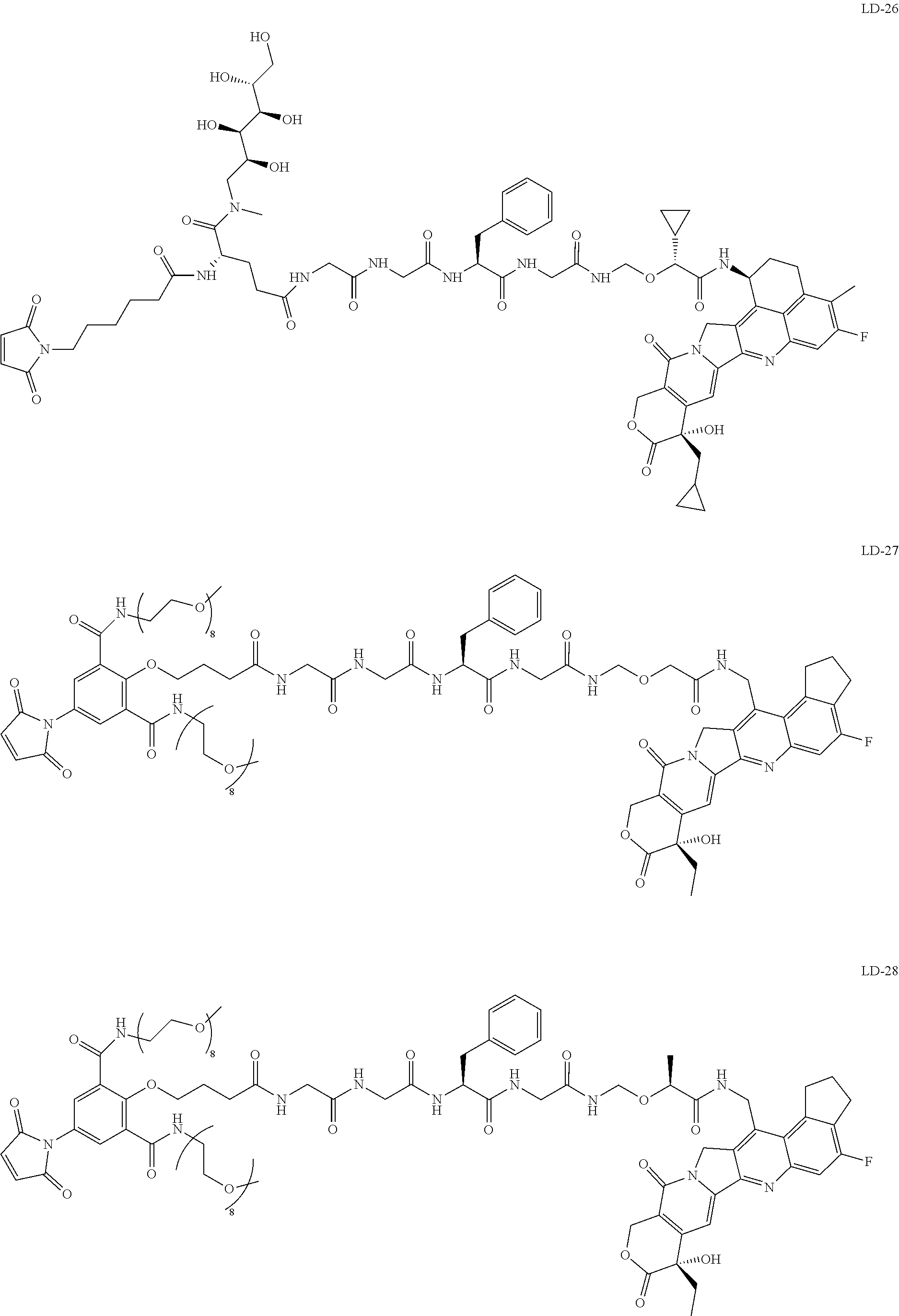

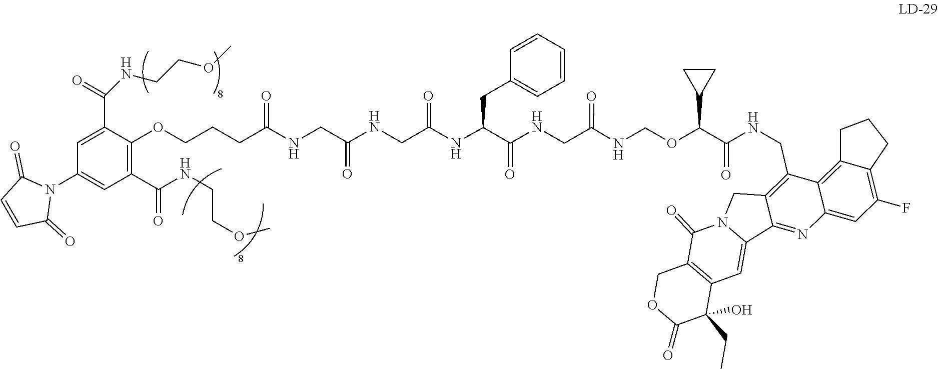

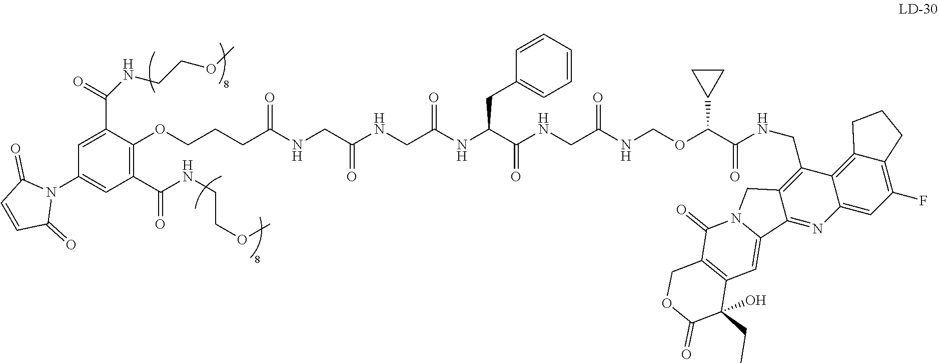

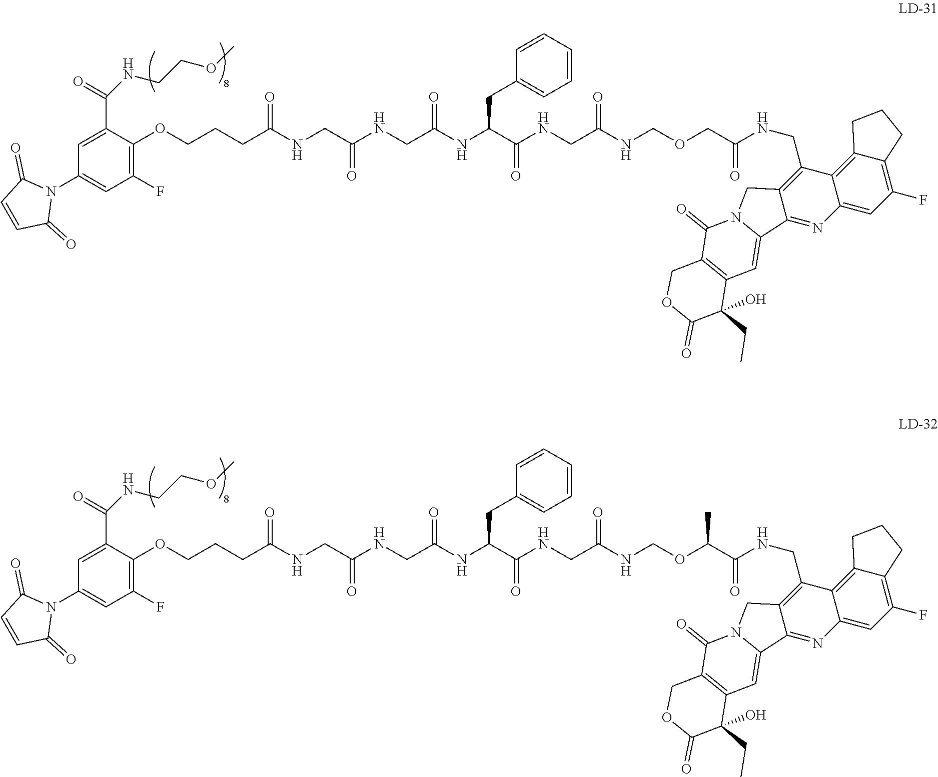

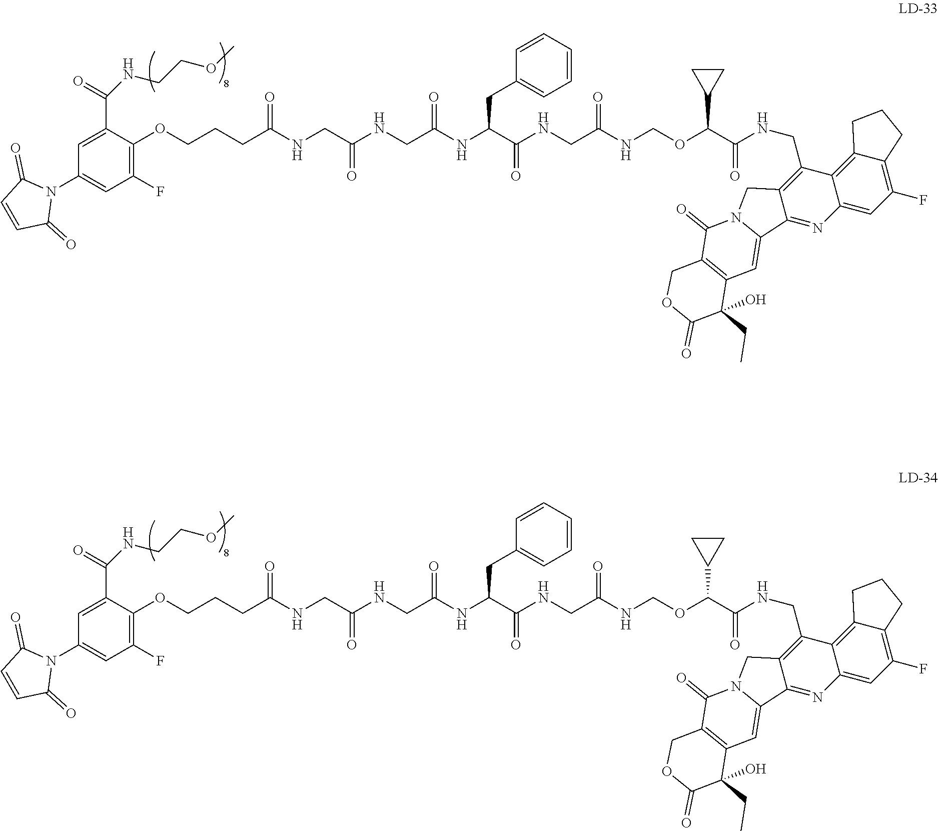

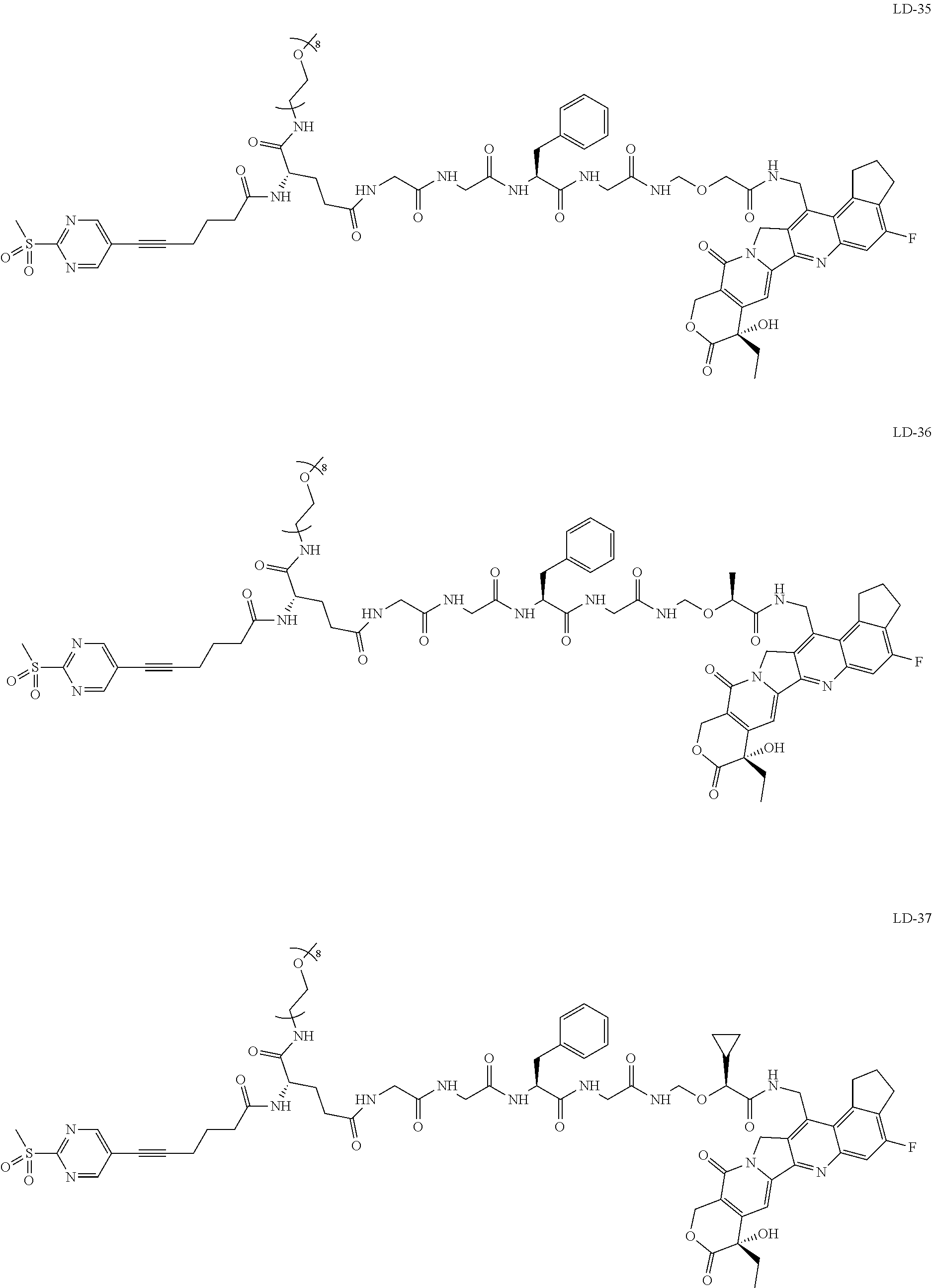

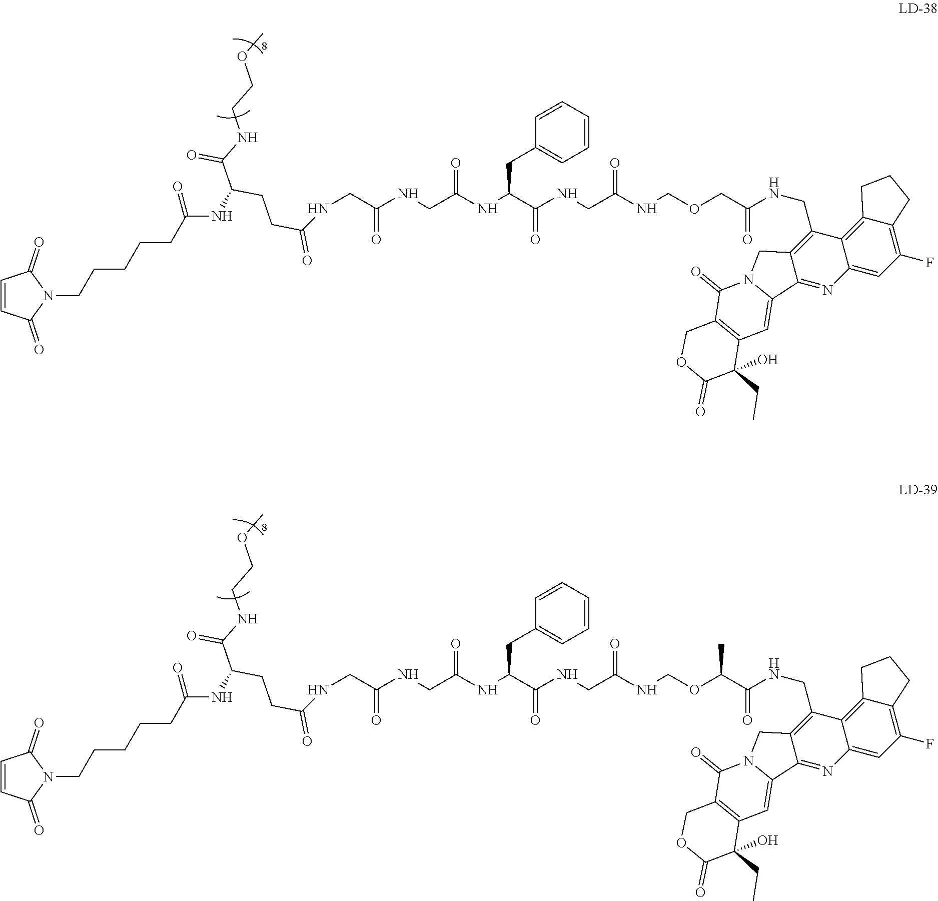

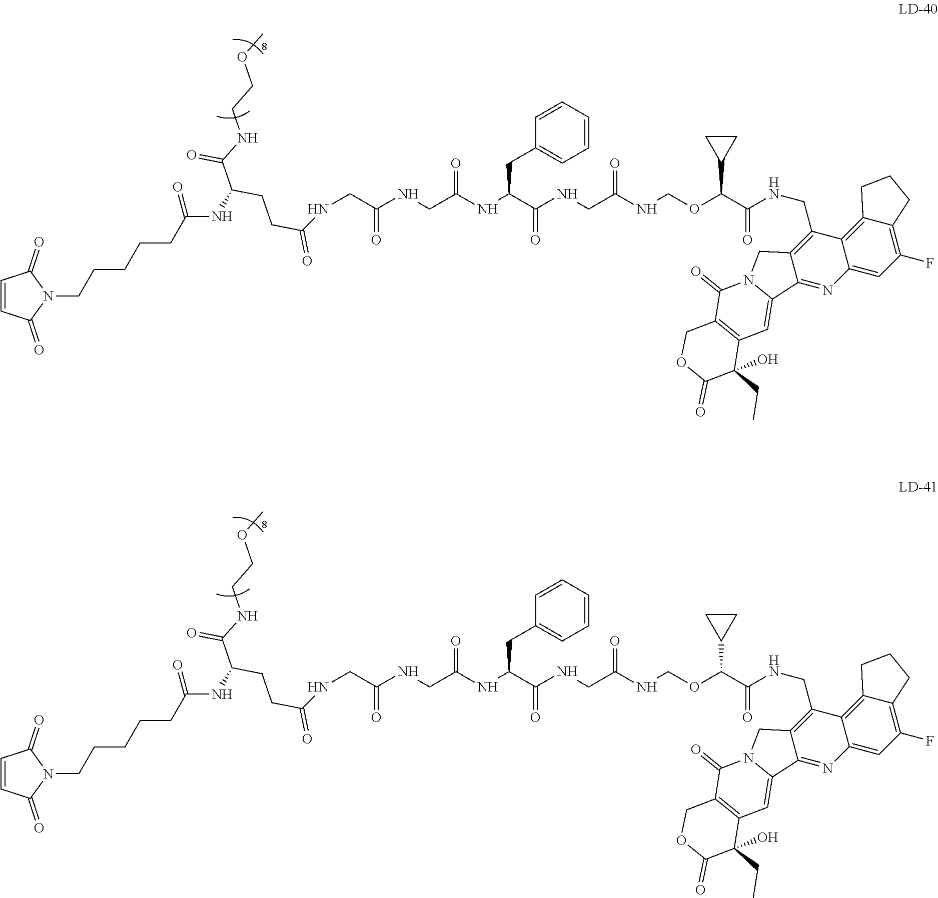

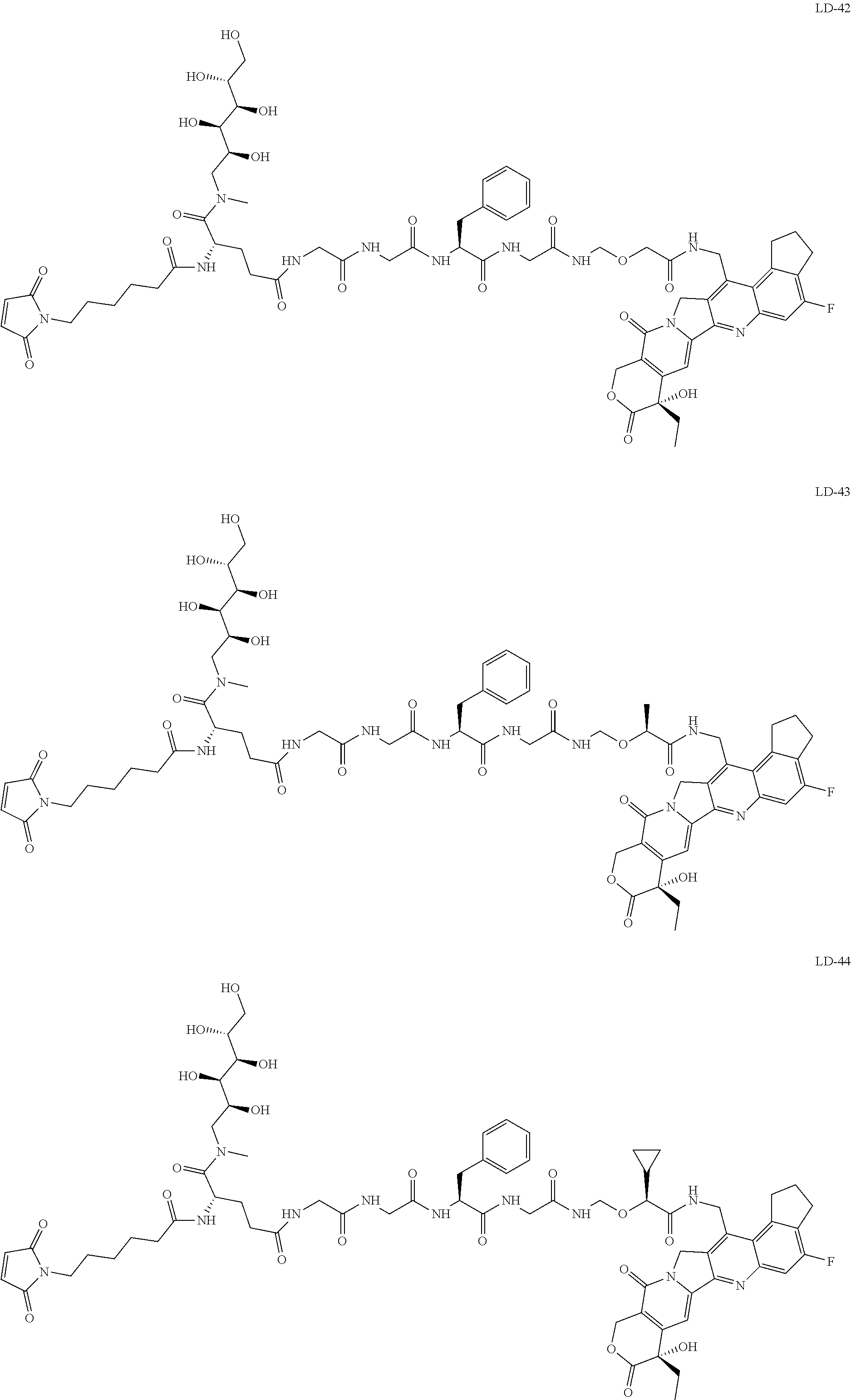

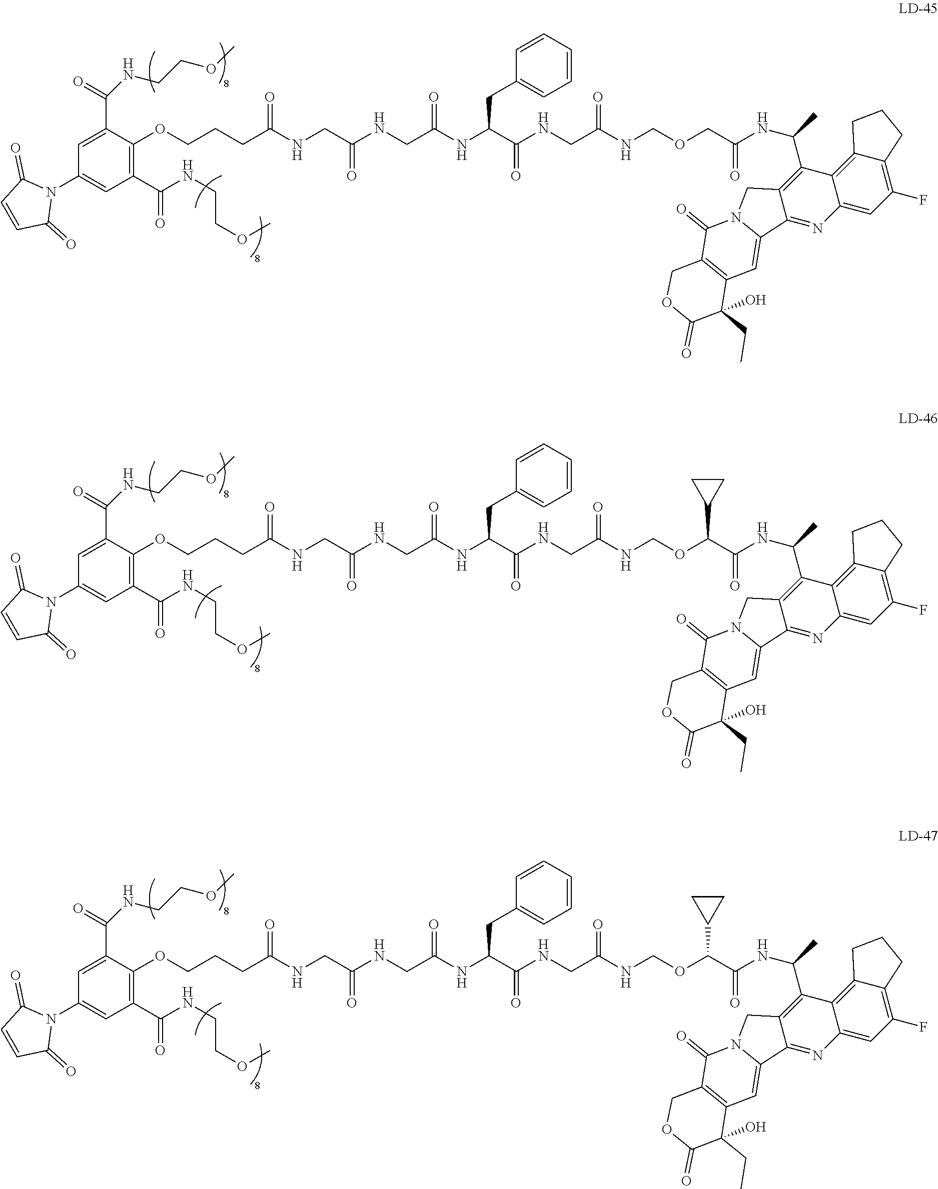

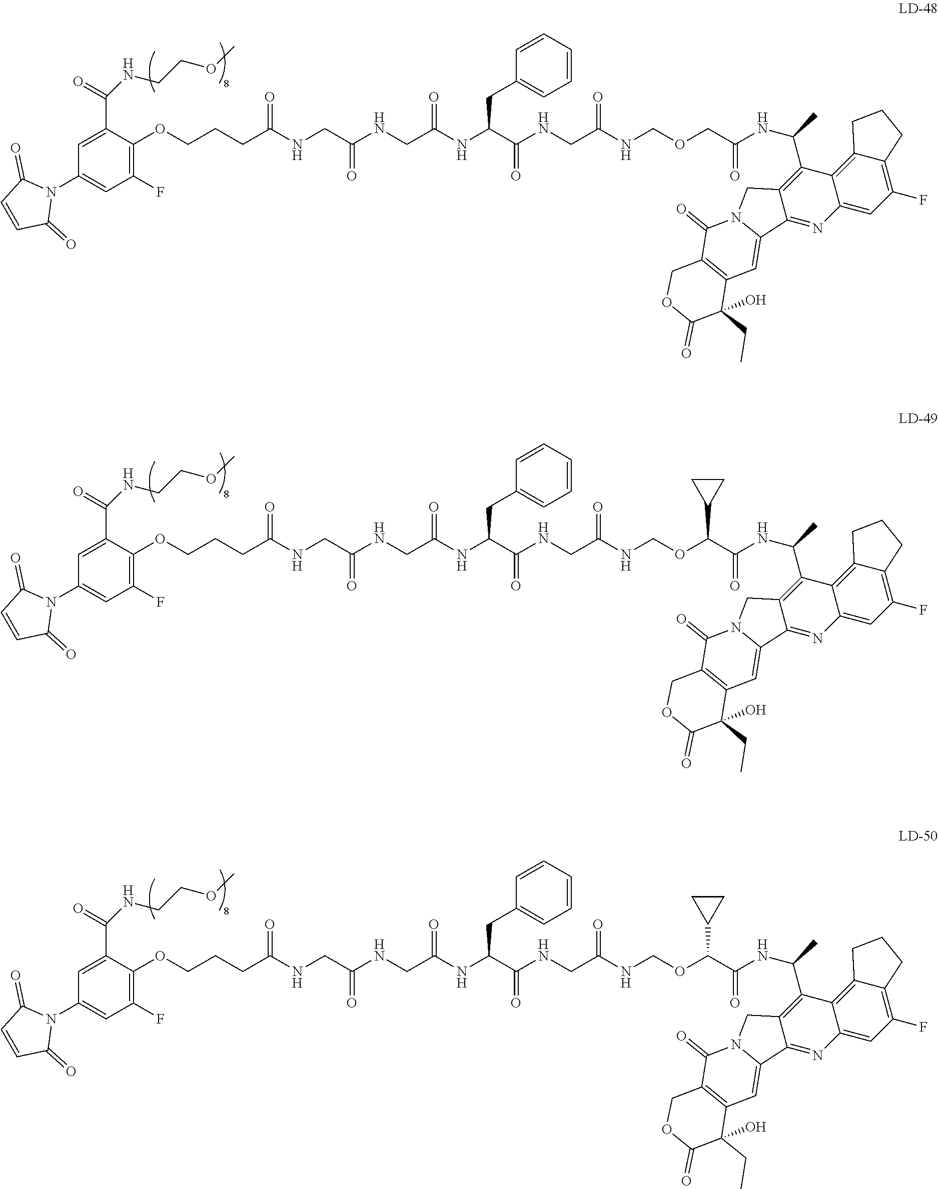

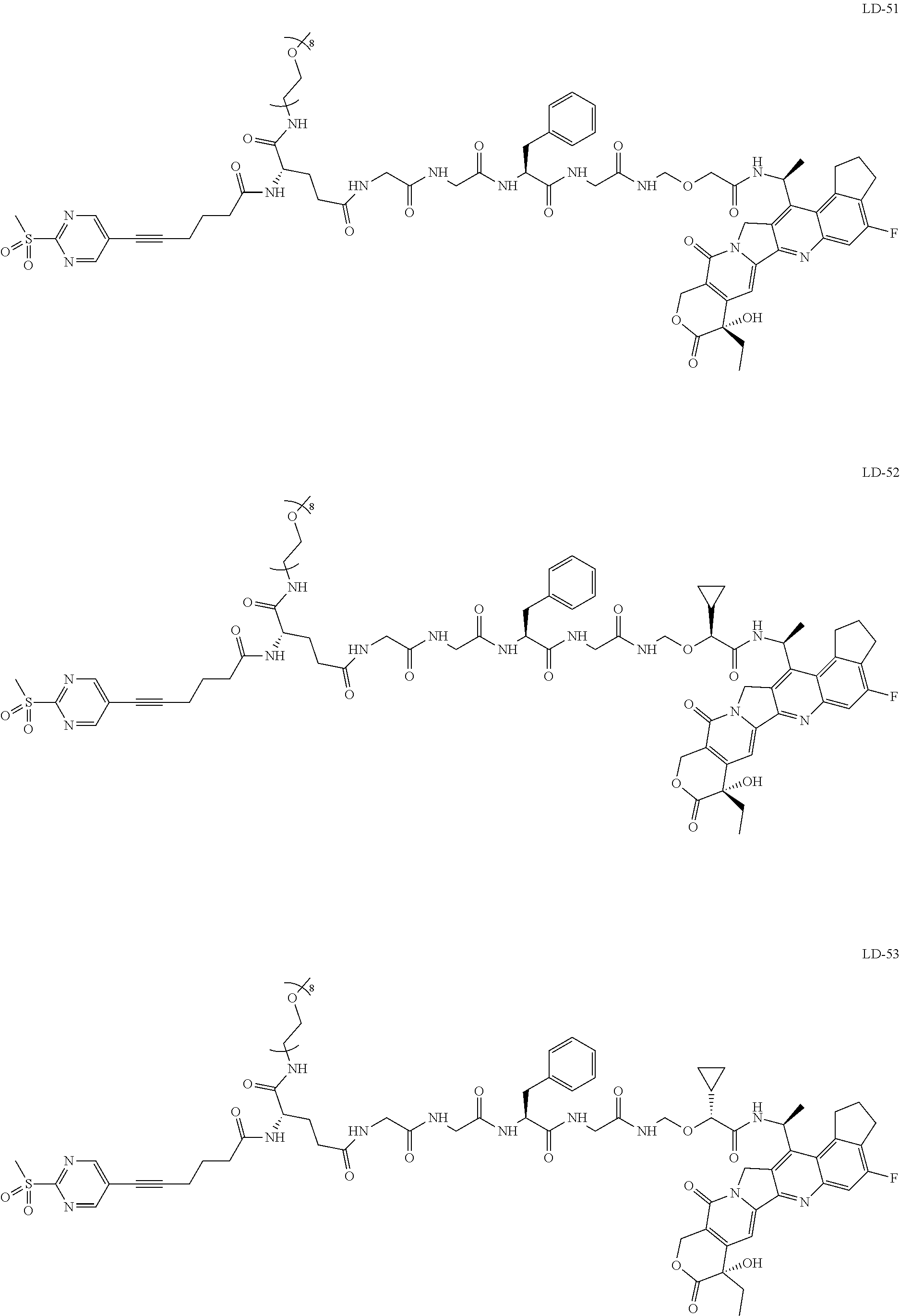

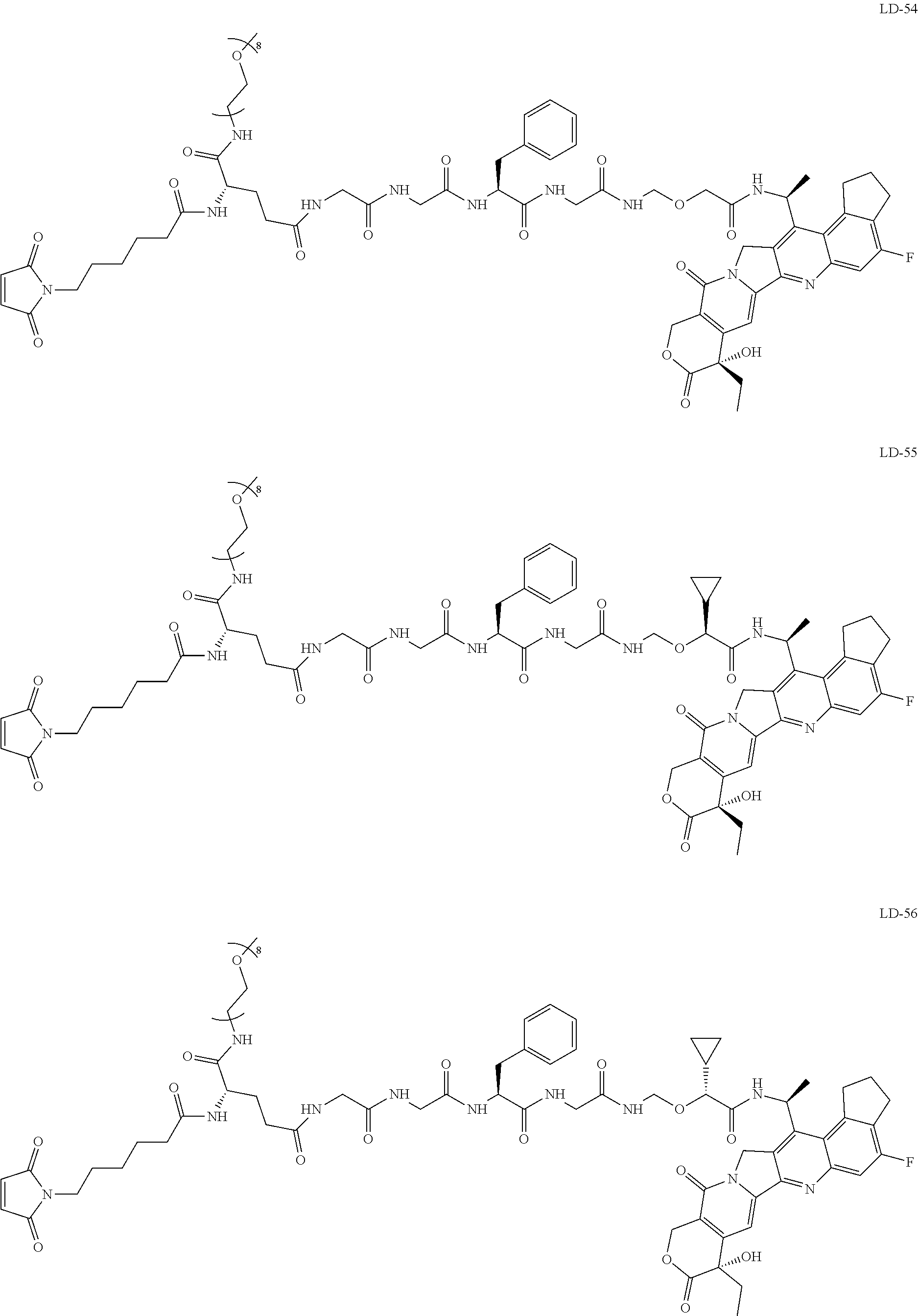

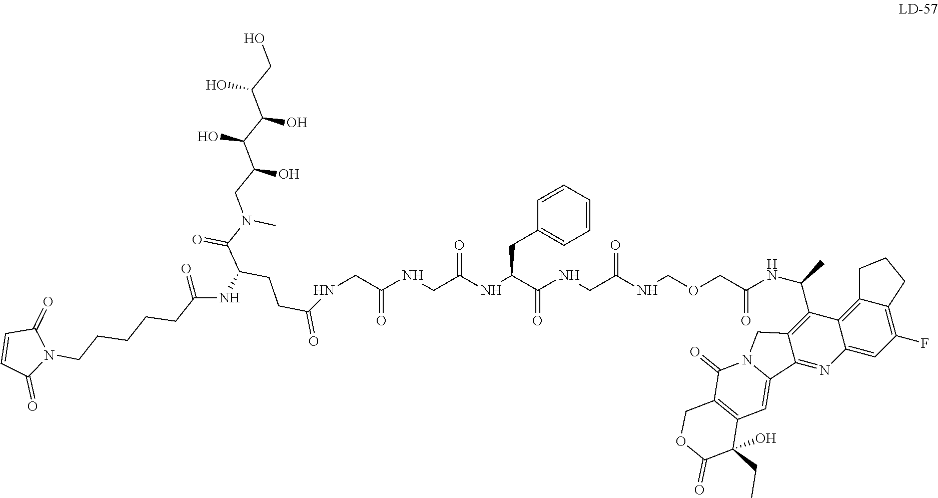 [SMILES: CC[C@@]1(O)C(=O)OCc2c1cc1n(c2=O)Cc2c-1nc1cc(F)c3c(c1c2[C@H](C)NC(=O)COCNC(=O)CNC(=O)[C@H](Cc1ccccc1)NC(=O)CNC(=O)CNC(=O)CC[C@H](NC(=O)CCCCCN1C(=O)C=CC1=O)C(=O)N(C)C[C@H](O)[C@@H](O)[C@H](O)[C@H](O)CO)CCC3]

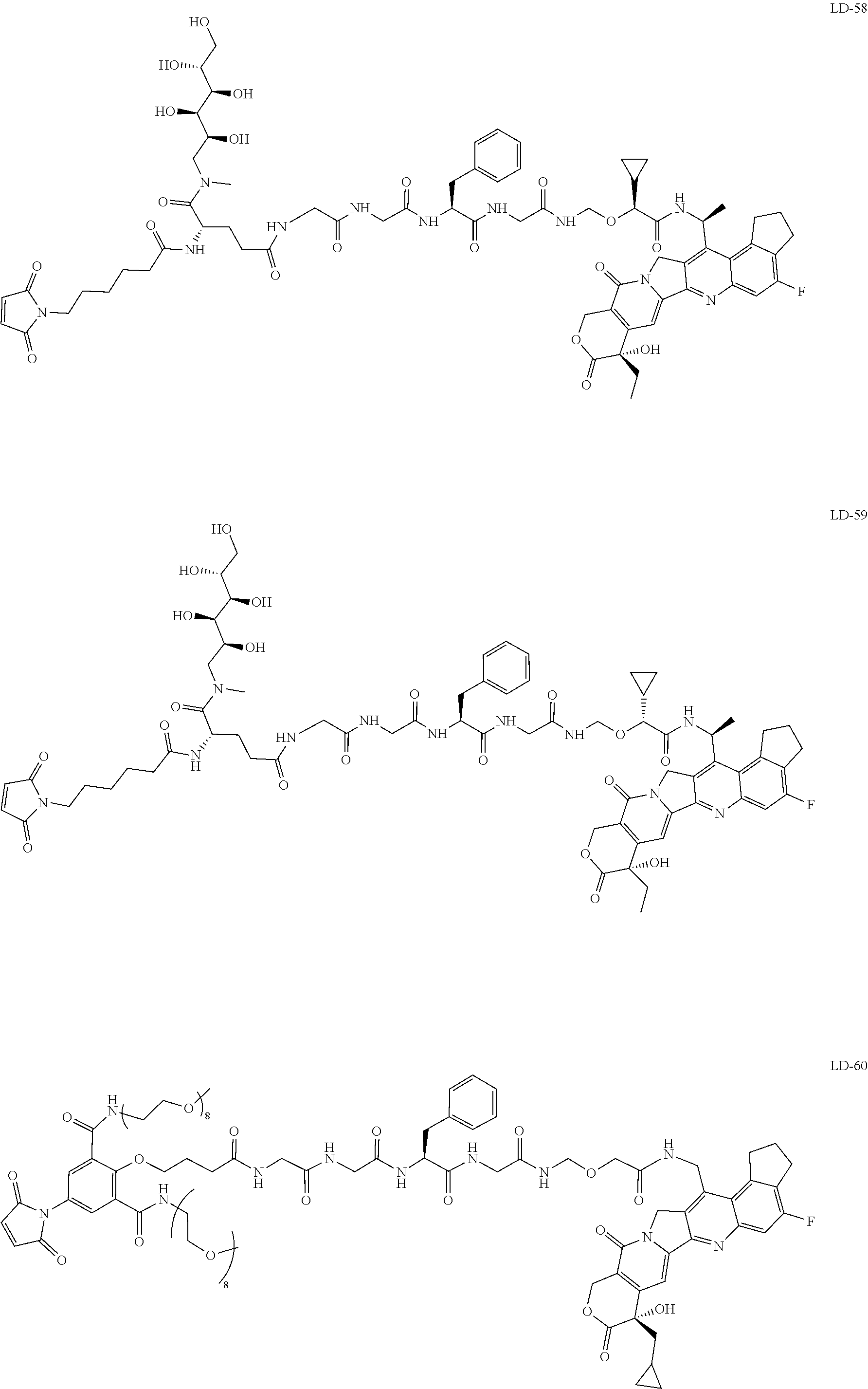

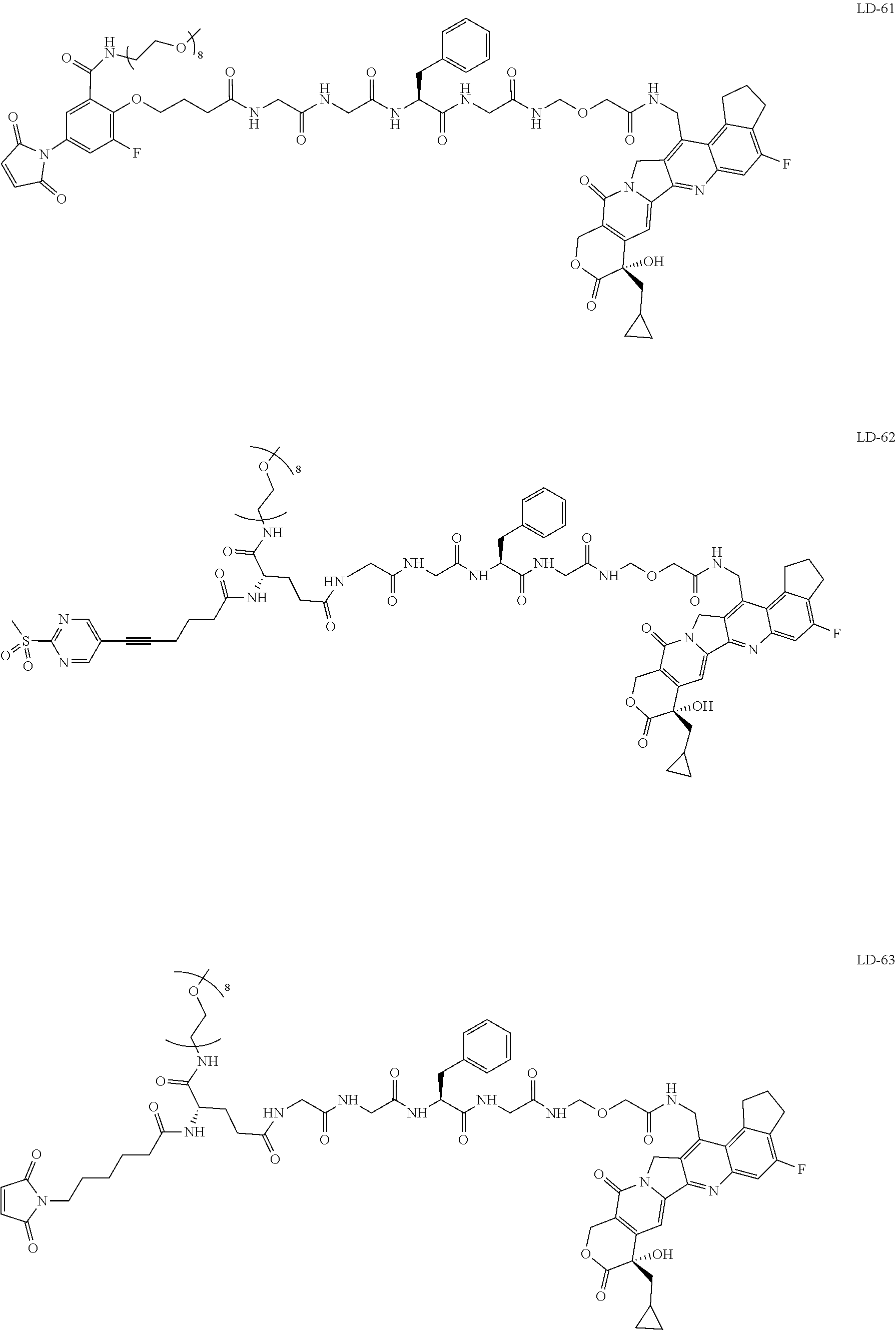

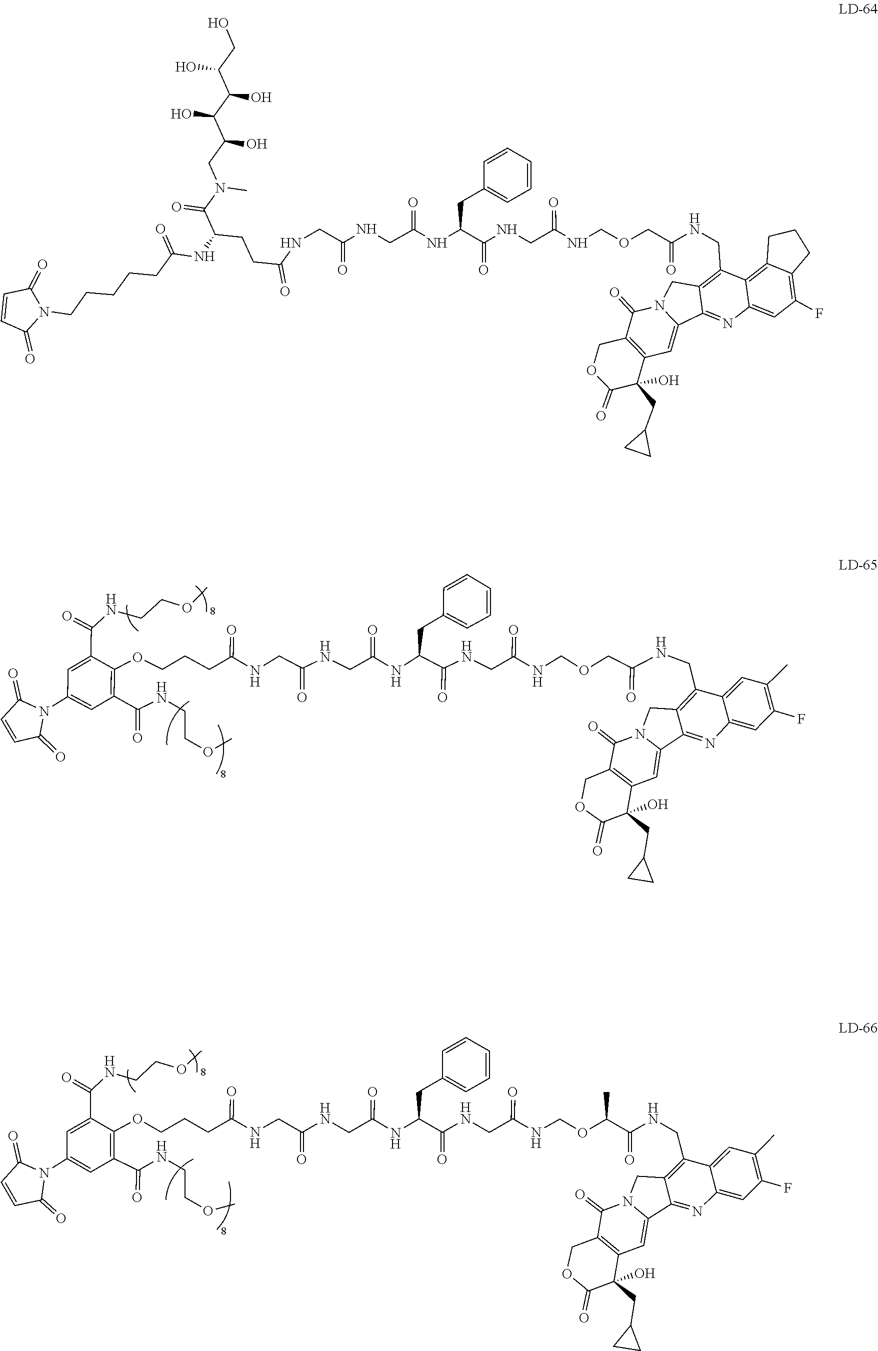

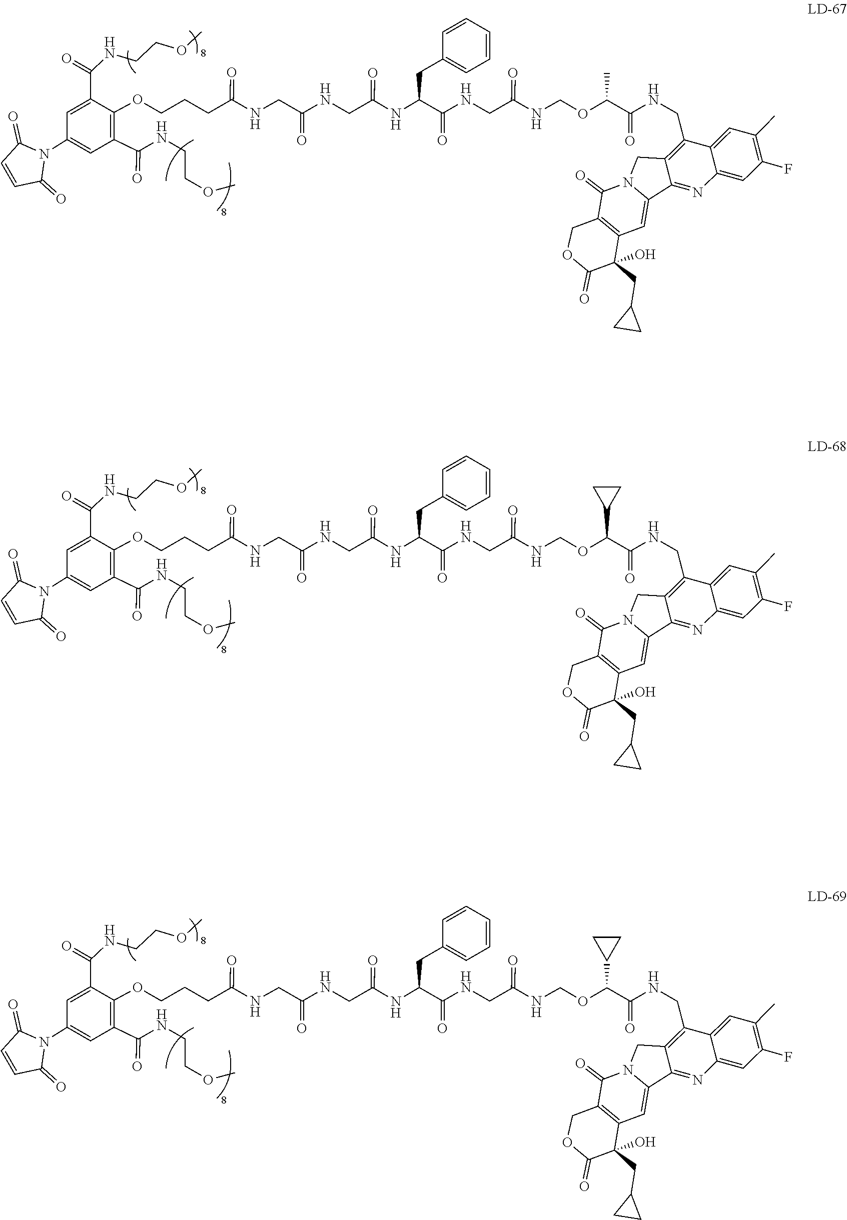

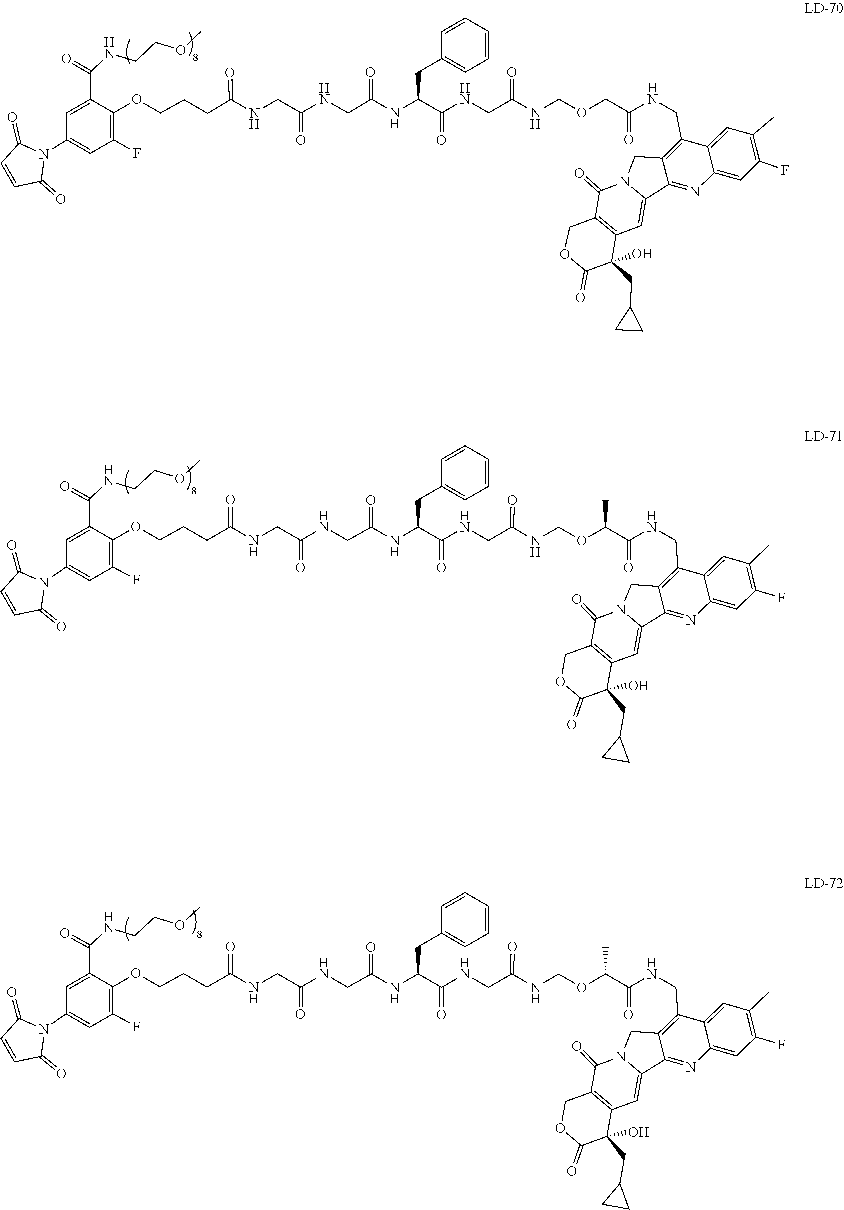

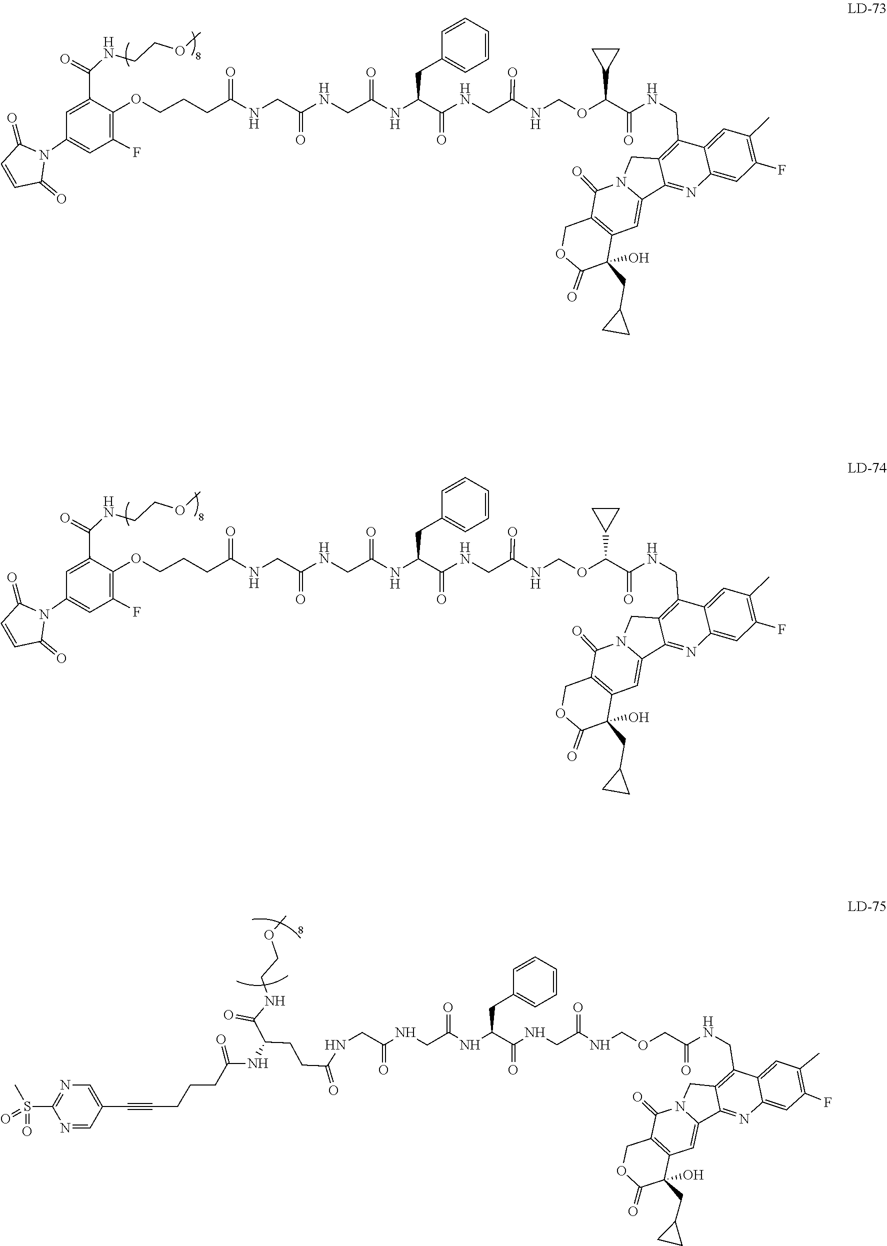

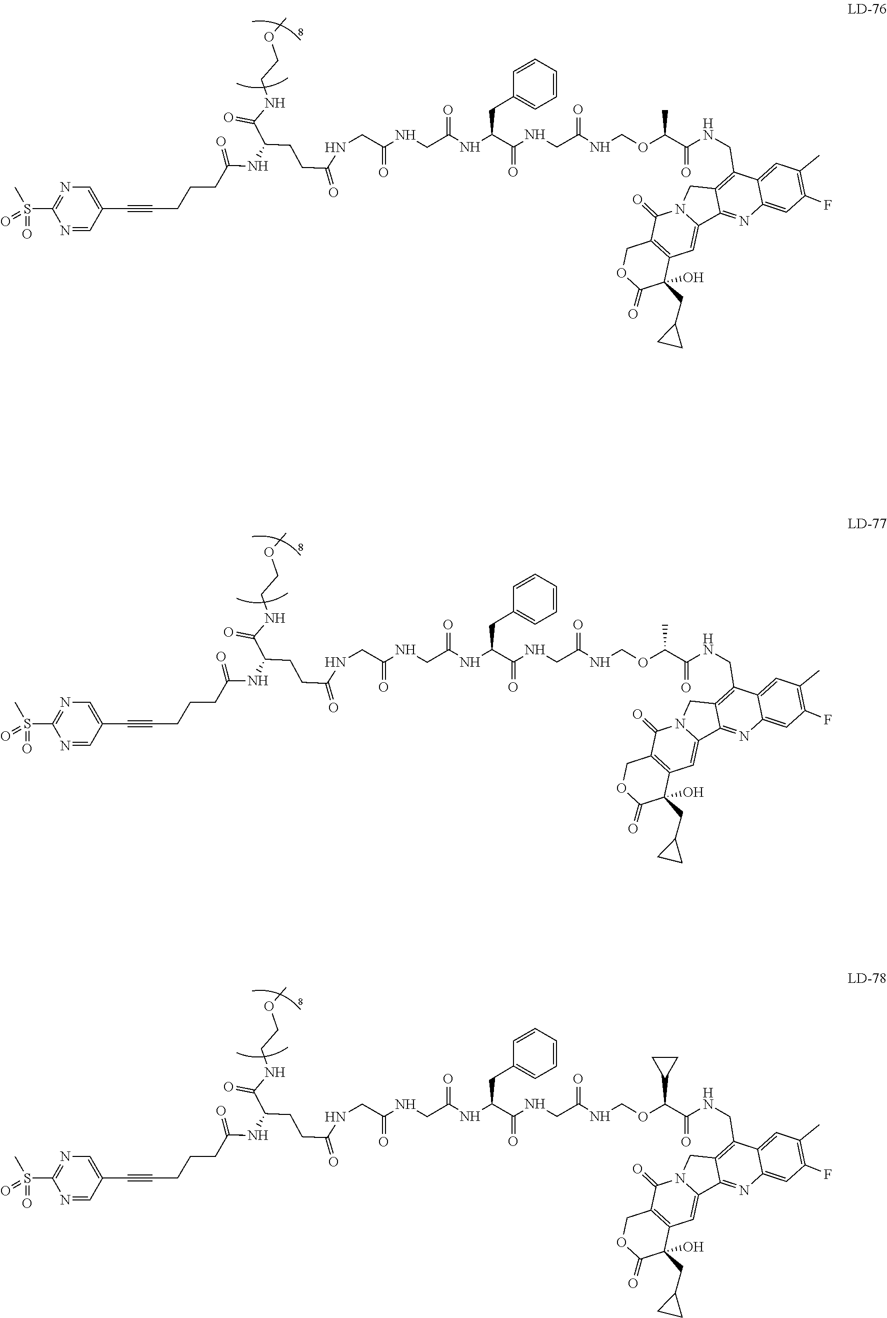

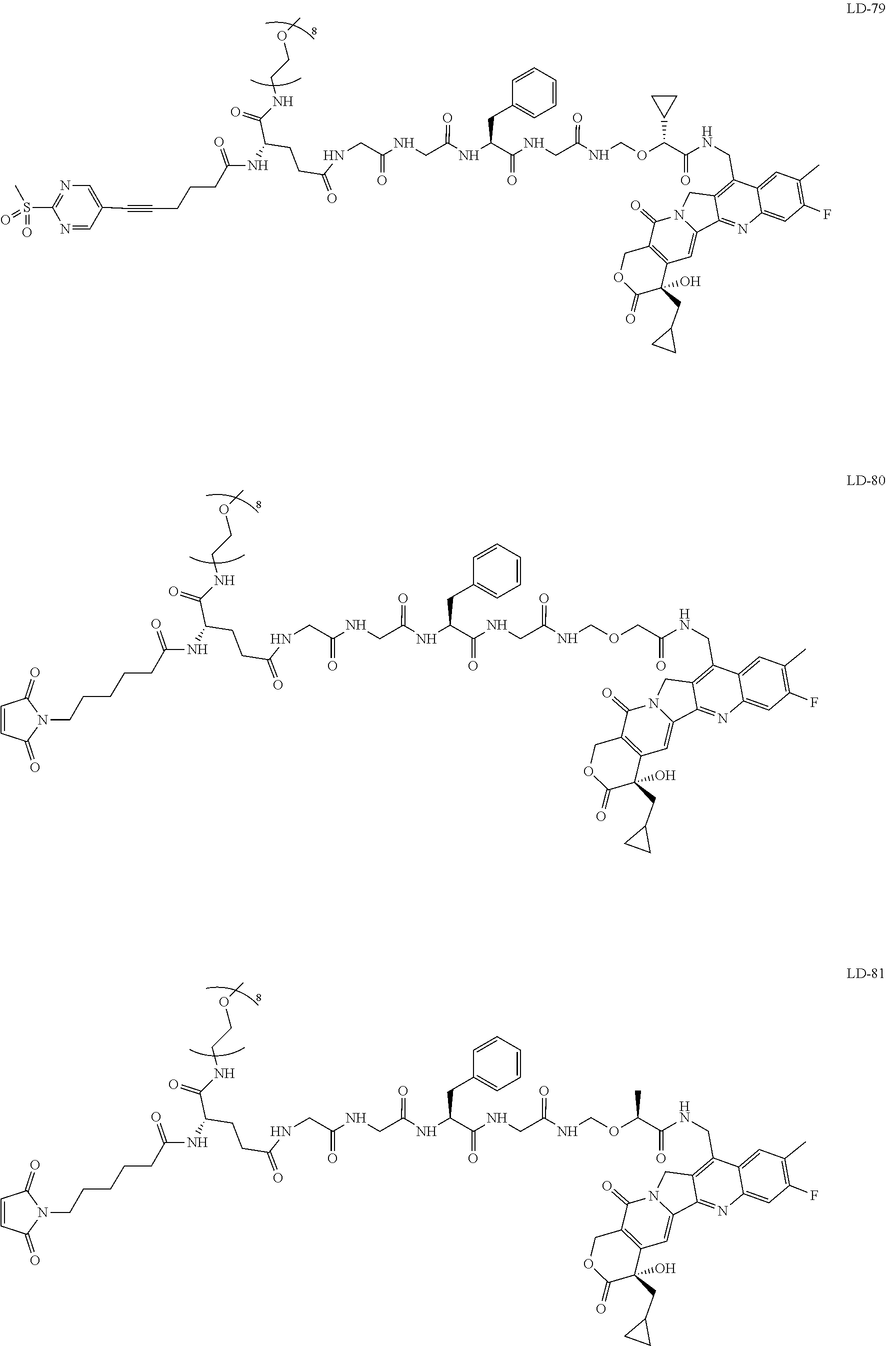

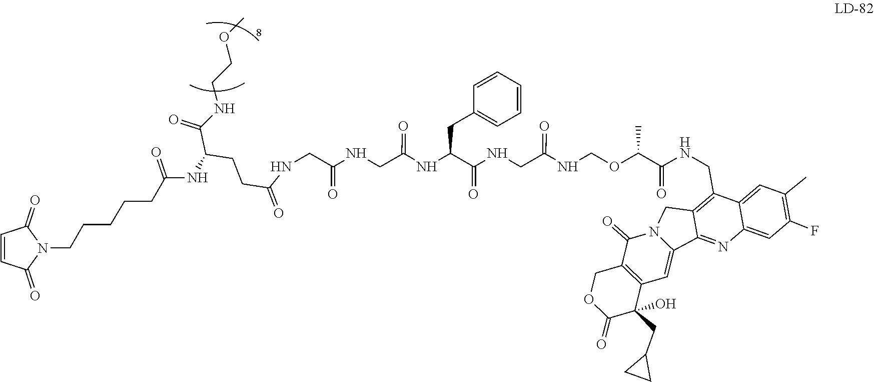

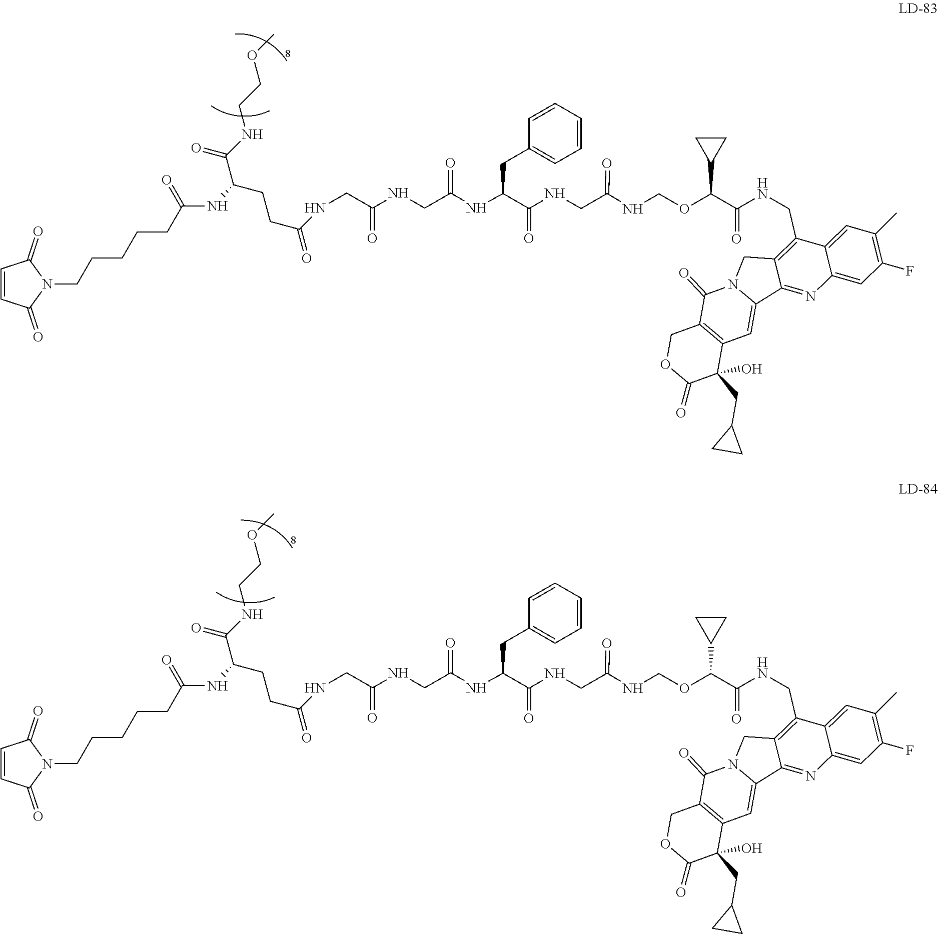

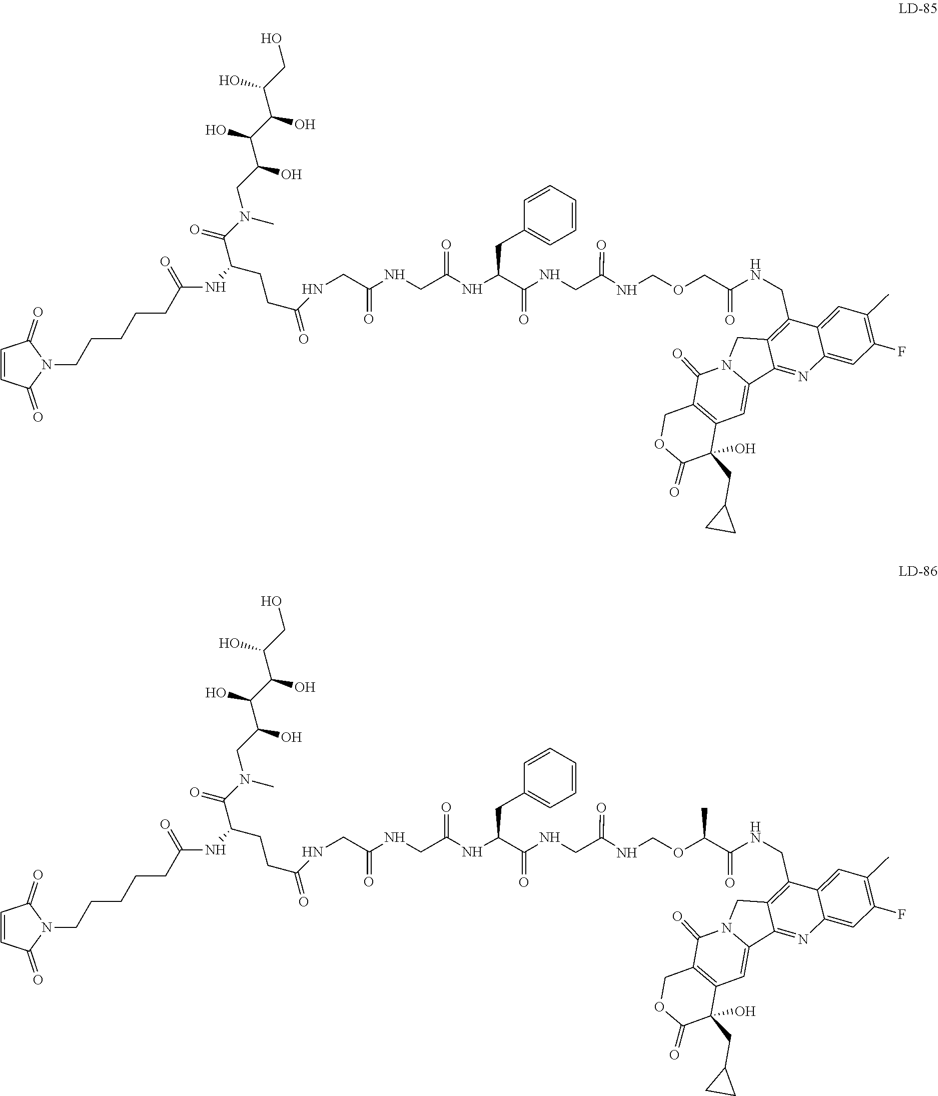 [SMILES: Cc1cc2c(CNC(=O)COCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1 ; Cc1cc2c(CNC(=O)[C@H](C)OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1]

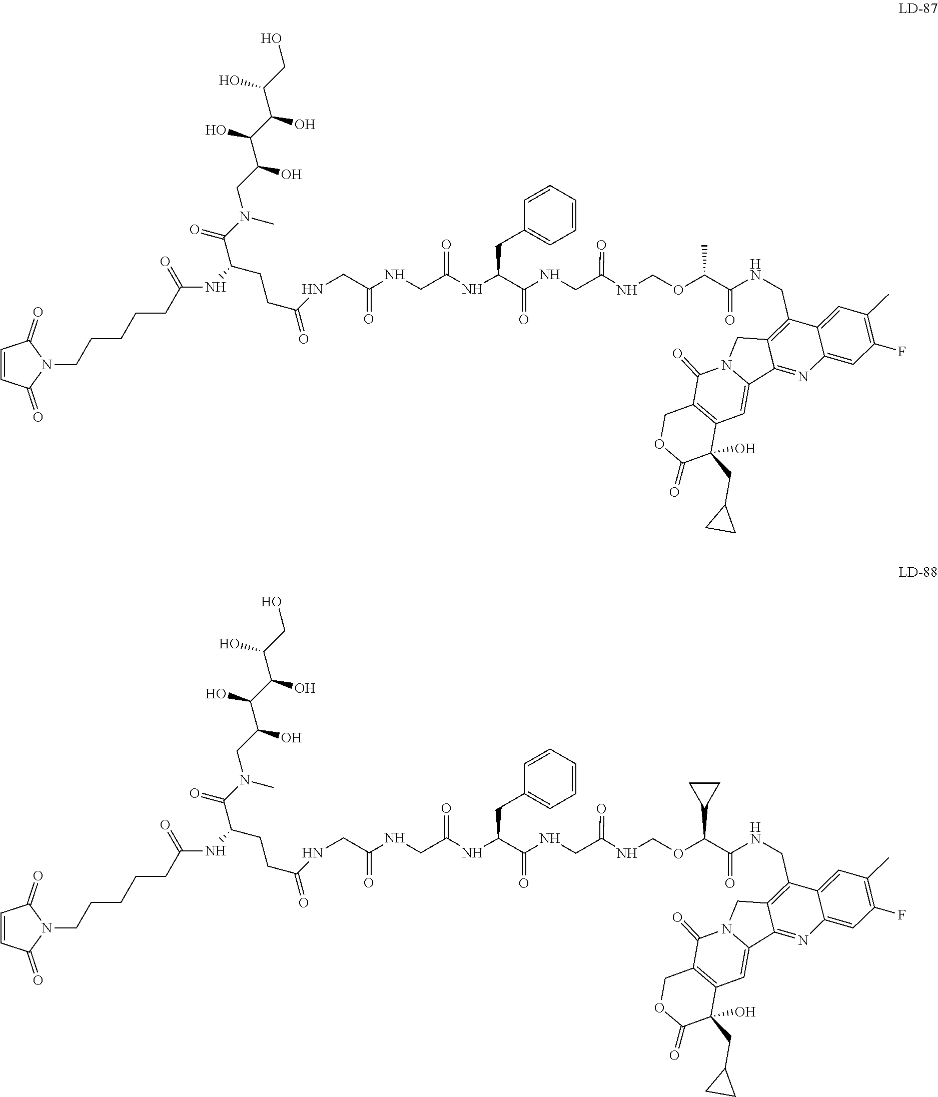 [SMILES: Cc1cc2c(CNC(=O)[C@@H](C)OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)c3c(nc2cc1F)-c1cc2c(c(=O)n1C3)COC(=O)[C@]2(O)CC1CC1 ; Cc1cc2c(CNC(=O)[C@@H](OCNC(=O)CNC(=O)[C@H](Cc3ccccc3)NC(=O)CNC(=O)CNC(=O)CC[C@H](NC(=O)CCCCCN3C(=O)C=CC3=O)C(=O)N(C)C[C@H](O)[C@@H](O)[C@H](O)[C@H](O)CO)C3CC3)c3c(nc2cc1F)-c1cc2c(c(=O)n1C3)COC(=O)[C@]2(O)CC1CC1]

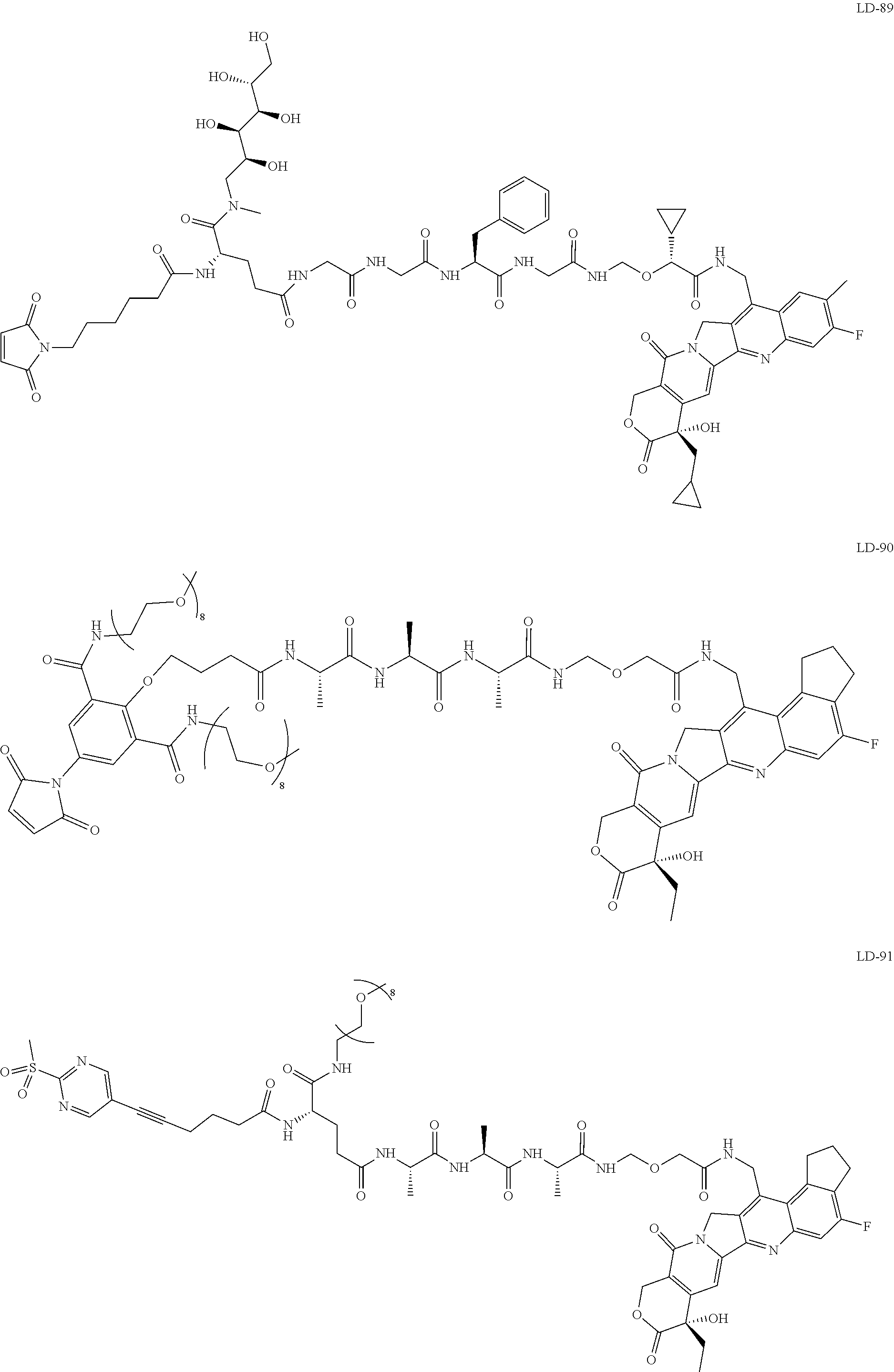

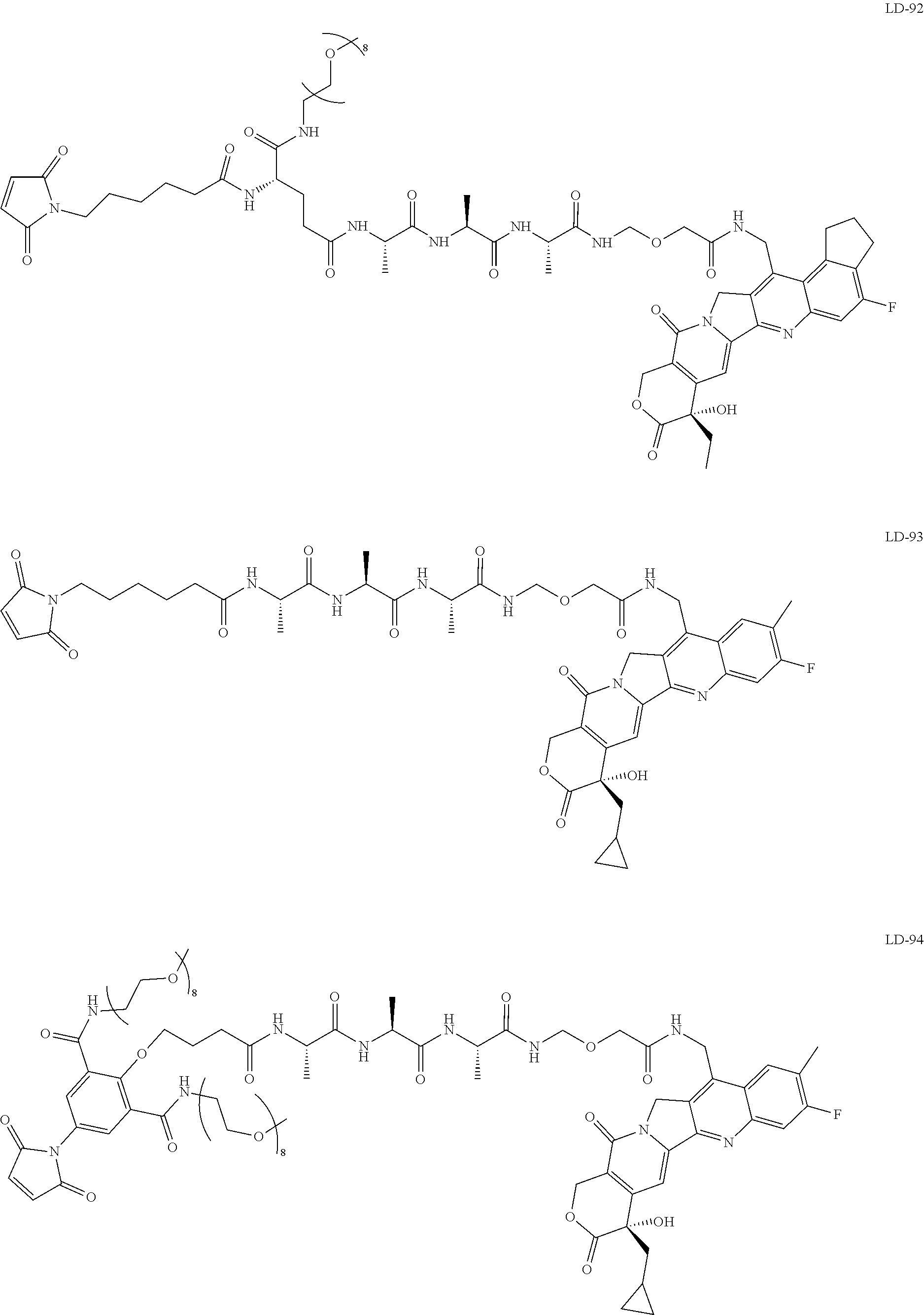

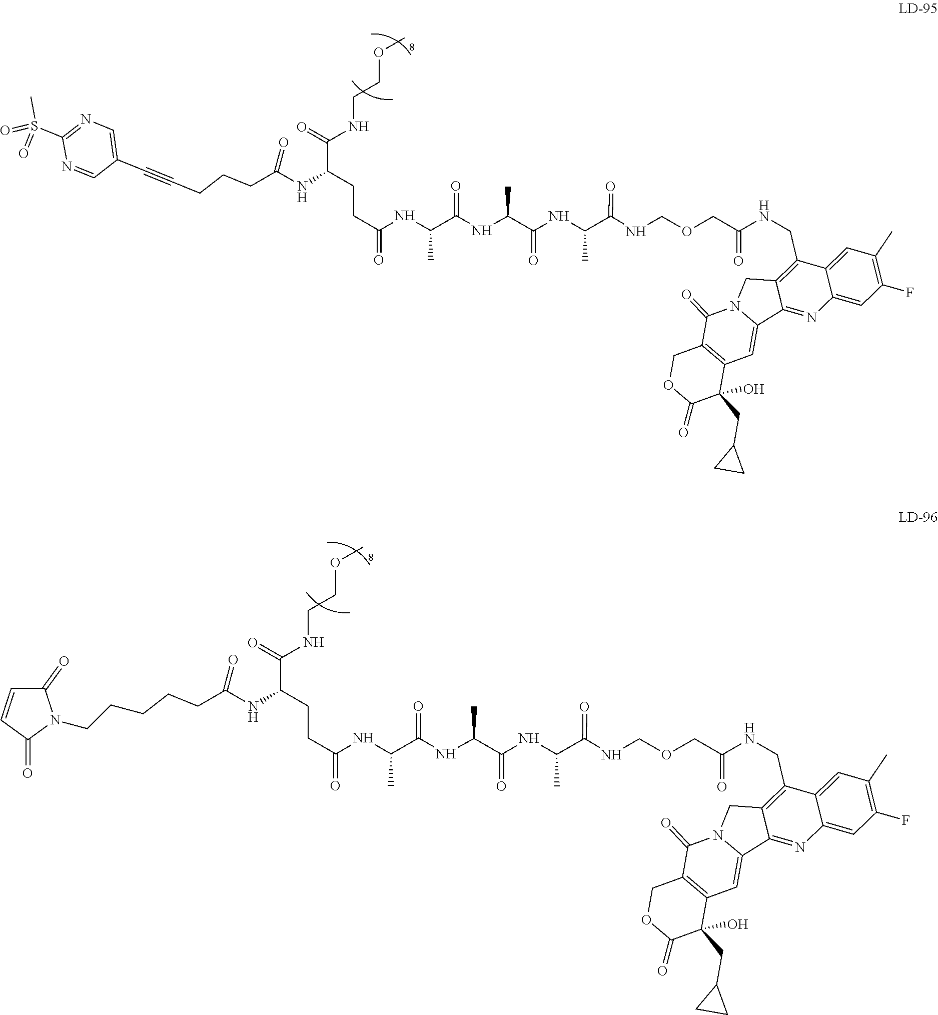

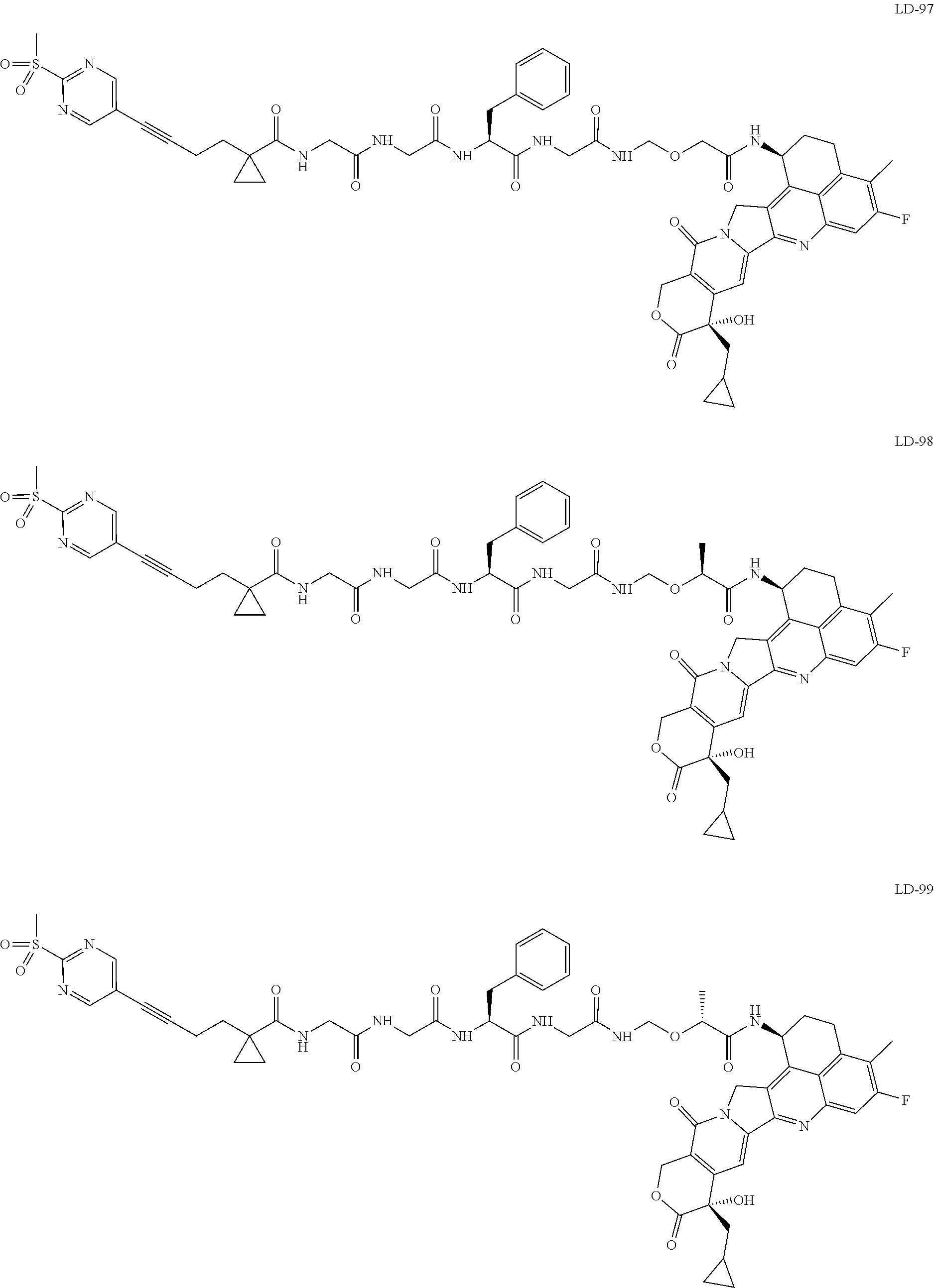 [SMILES: Cc1c(F)cc2nc3c(c4c2c1CC[C@@H]4NC(=O)COCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1 ; Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)C1(CCC#Cc2cnc(S(C)(=O)=O)nc2)CC1)Cn1c-3cc2c(c1=O)COC(=O)[C@]2(O)CC1CC1]

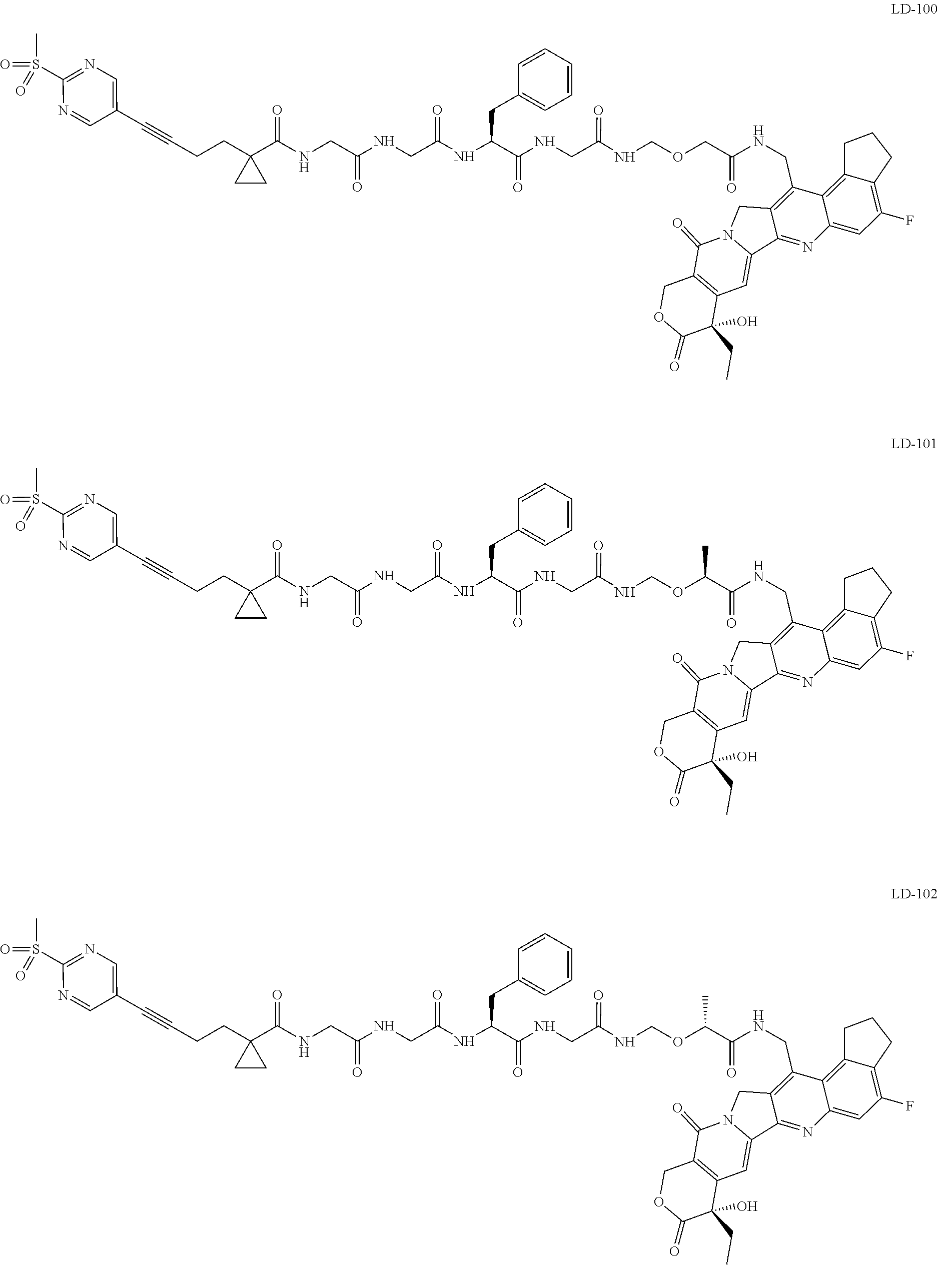 [SMILES: CC[C@@]1(O)C(=O)OCc2c1cc1n(c2=O)Cc2c-1nc1cc(F)c3c(c1c2CNC(=O)COCNC(=O)CNC(=O)[C@H](Cc1ccccc1)NC(=O)CNC(=O)CNC(=O)C1(CCC#Cc2cnc(S(C)(=O)=O)nc2)CC1)CCC3 ; CC[C@@]1(O)C(=O)OCc2c1cc1n(c2=O)Cc2c-1nc1cc(F)c3c(c1c2CNC(=O)[C@H](C)OCNC(=O)CNC(=O)[C@H](Cc1ccccc1)NC(=O)CNC(=O)CNC(=O)C1(CCC#Cc2cnc(S(C)(=O)=O)nc2)CC1)CCC3 ; CC[C@@]1(O)C(=O)OCc2c1cc1n(c2=O)Cc2c-1nc1cc(F)c3c(c1c2CNC(=O)[C@@H](C)OCNC(=O)CNC(=O)[C@H](Cc1ccccc1)NC(=O)CNC(=O)CNC(=O)C1(CCC#Cc2cnc(S(C)(=O)=O)nc2)CC1)CCC3]

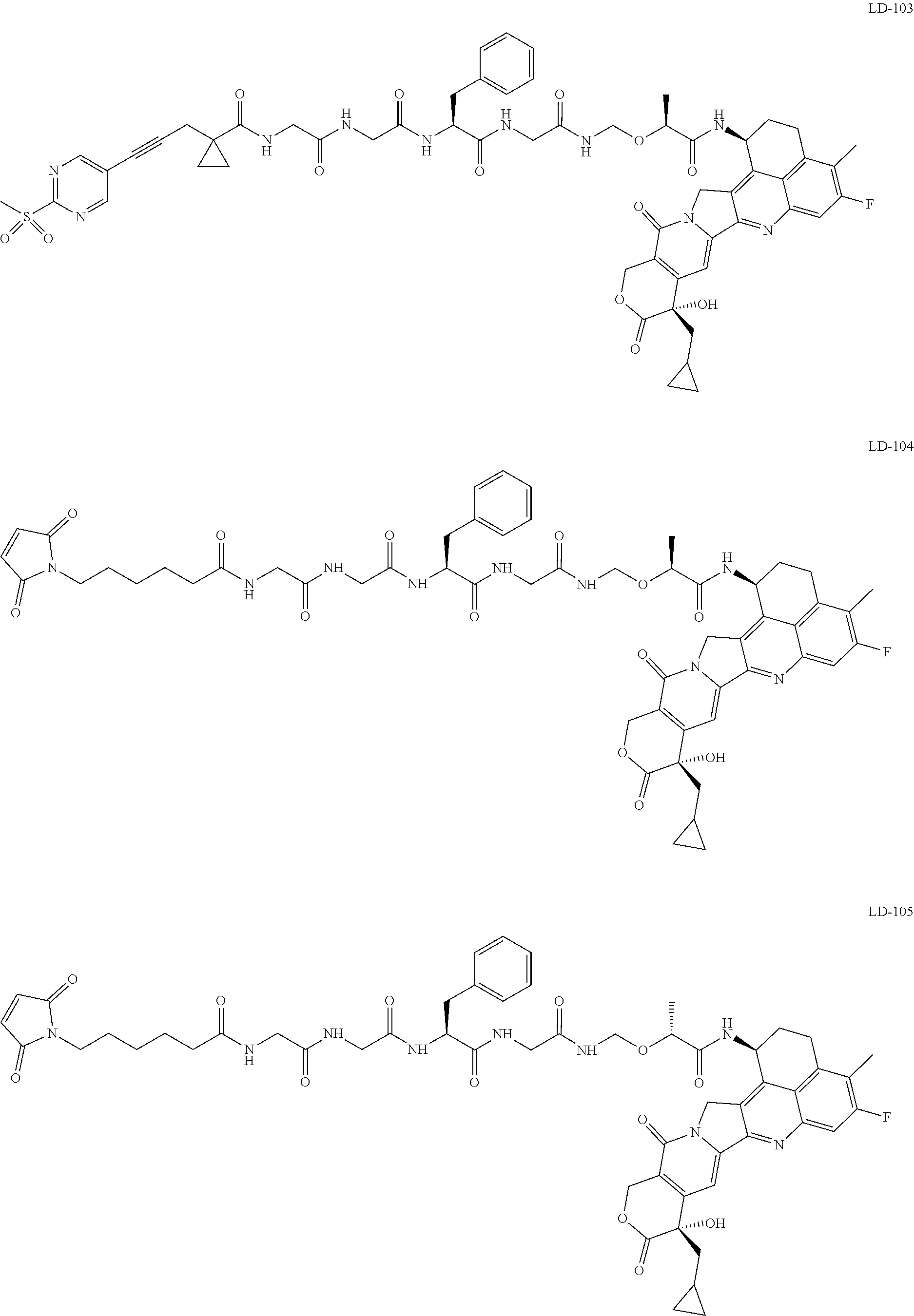 [SMILES: Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)C1(CC#Cc2cnc(S(C)(=O)=O)nc2)CC1)Cn1c-3cc2c(c1=O)COC(=O)[C@]2(O)CC1CC1 ; Cc1c(F)cc2nc3c(c4c2c1CC[C@@H]4NC(=O)[C@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1 ; Cc1c(F)cc2nc3c(c4c2c1CC[C@@H]4NC(=O)[C@@H](C)OCNC(=O)CNC(=O)[C@H](Cc1ccccc1)NC(=O)CNC(=O)CNC(=O)CCCCCN1C(=O)C=CC1=O)Cn1c-3cc2c(c1=O)COC(=O)[C@]2(O)CC1CC1]

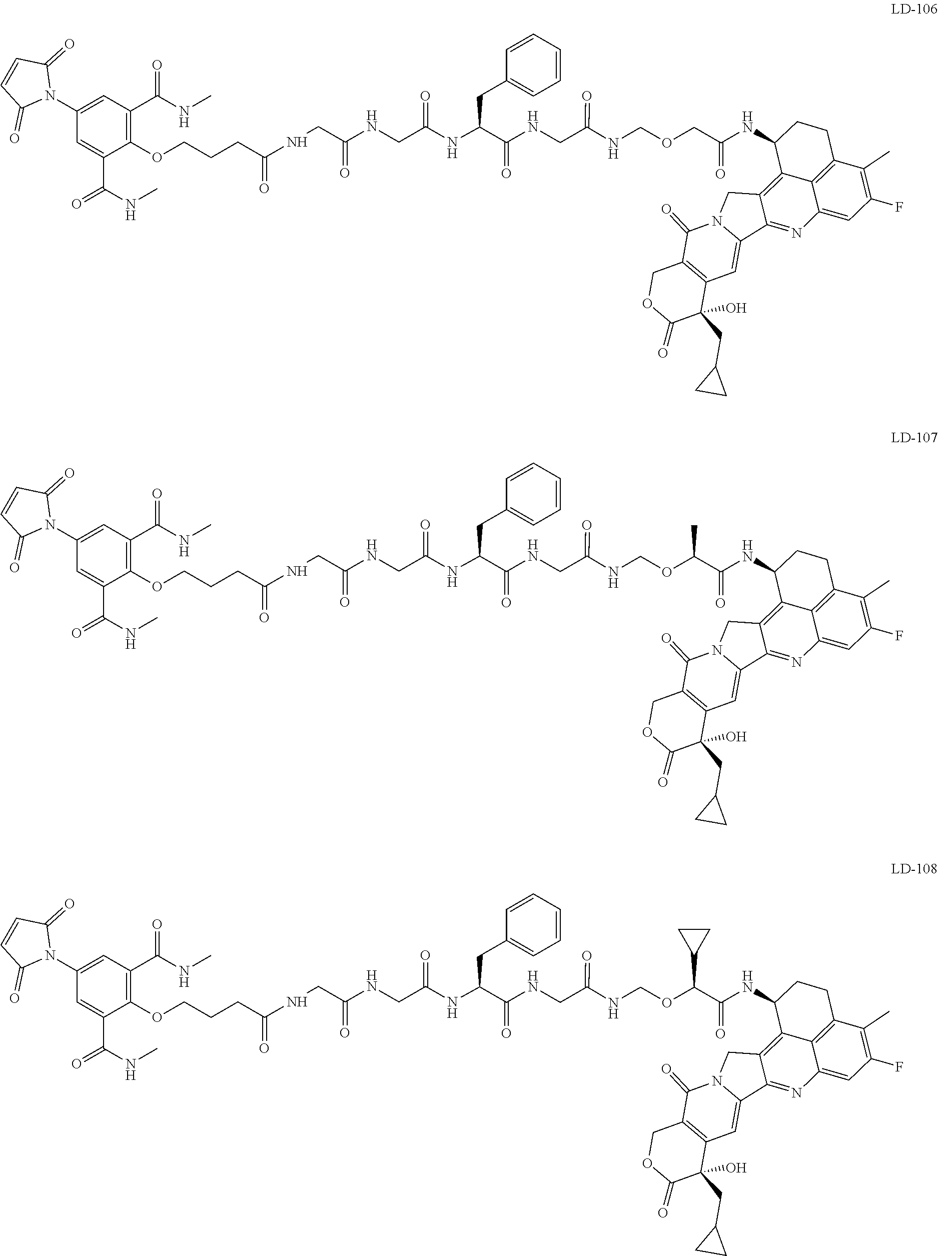

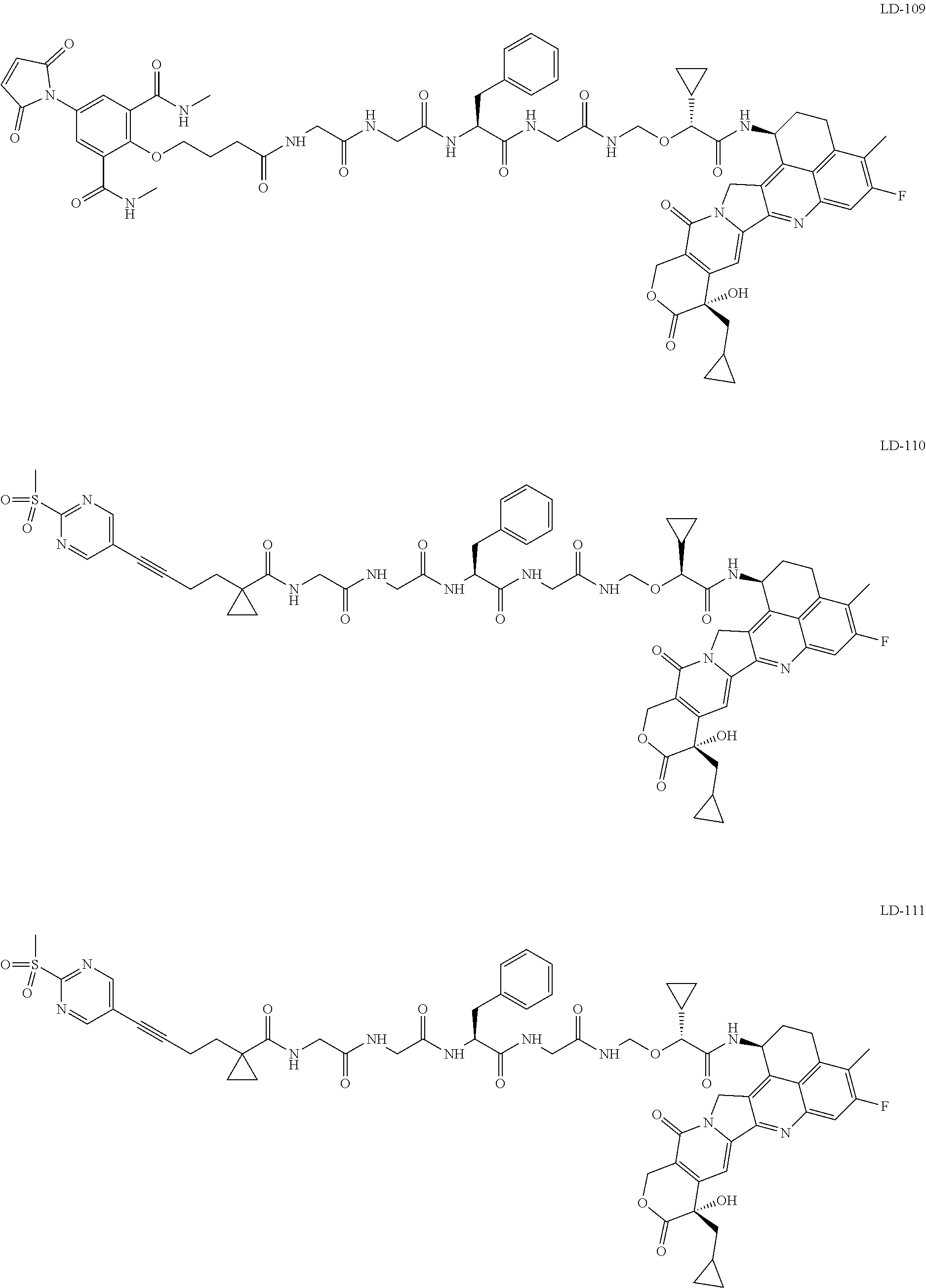

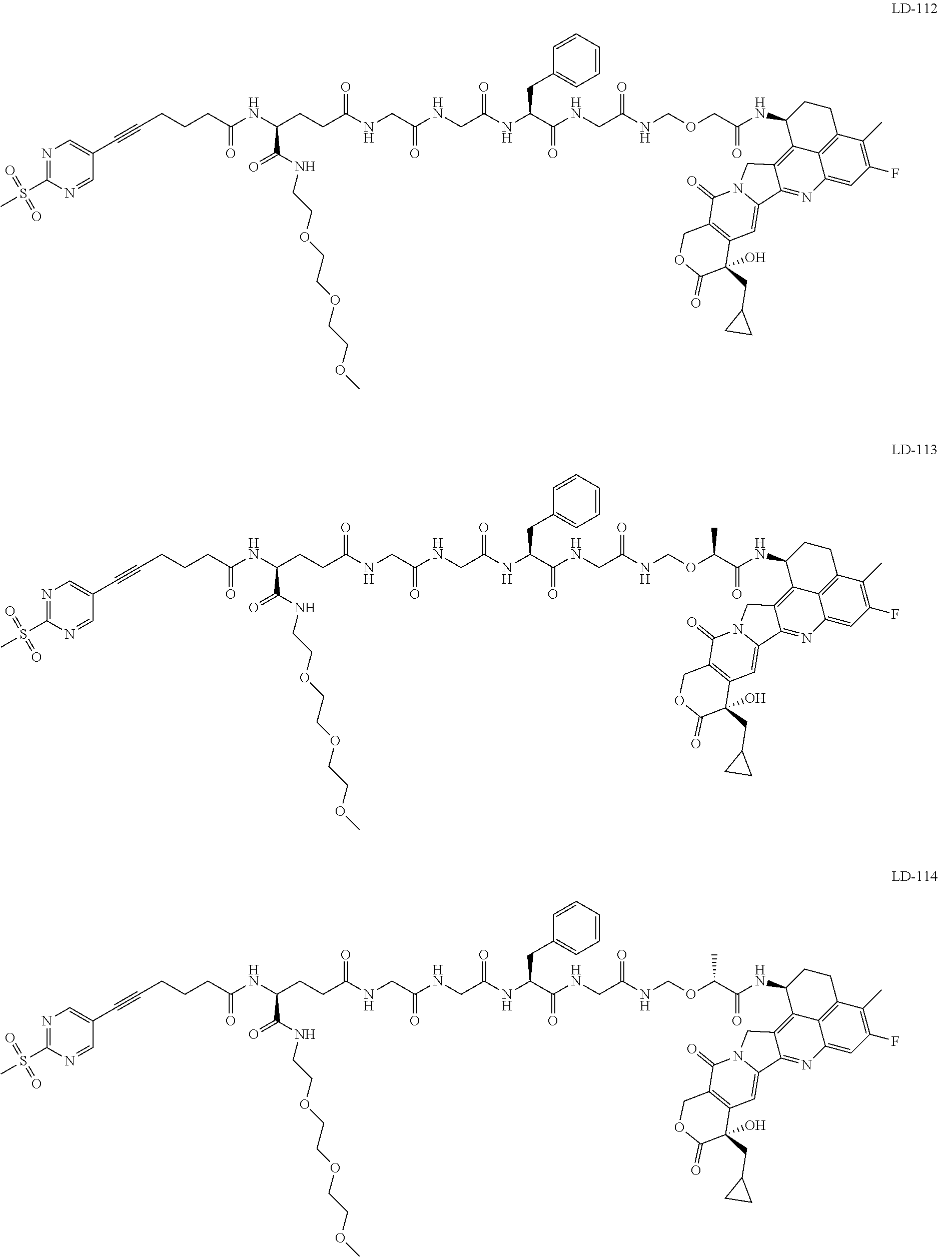

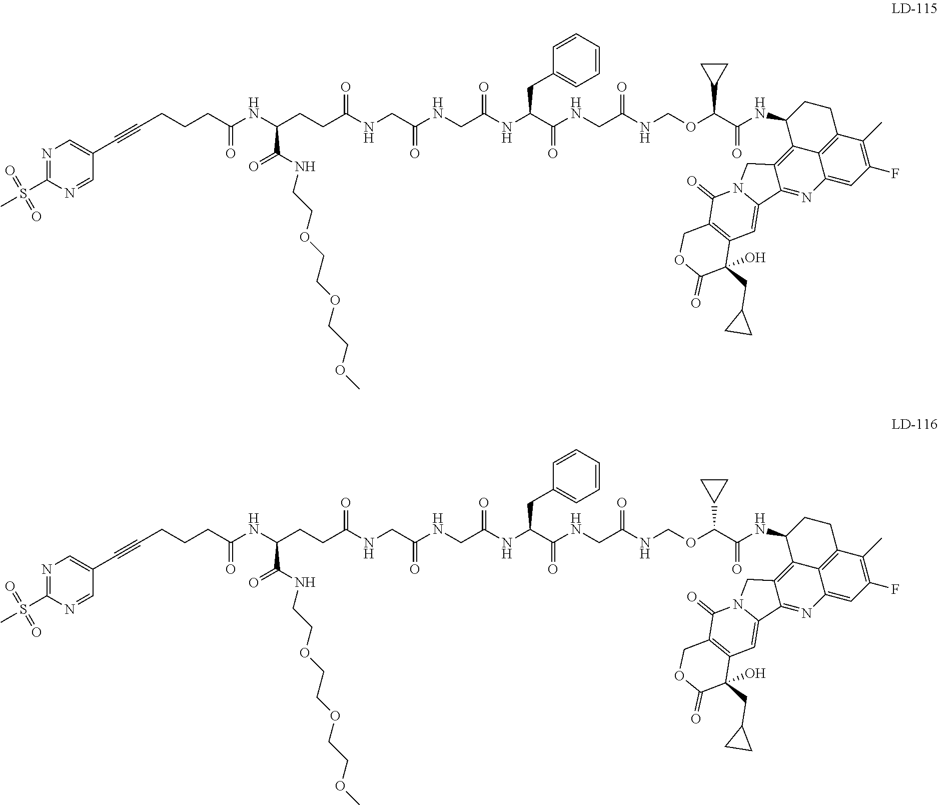 [SMILES: COCCOCCOCCNC(=O)[C@H](CCC(=O)NCC(=O)NCC(=O)N[C@@H](Cc1ccccc1)C(=O)NCC(=O)NCO[C@H](C(=O)N[C@H]1CCc2c(C)c(F)cc3nc4c(c1c23)Cn1c-4cc2c(c1=O)COC(=O)[C@]2(O)CC1CC1)C1CC1)NC(=O)CCCC#Cc1cnc(S(C)(=O)=O)nc1 ; COCCOCCOCCNC(=O)[C@H](CCC(=O)NCC(=O)NCC(=O)N[C@@H](Cc1ccccc1)C(=O)NCC(=O)NCO[C@@H](C(=O)N[C@H]1CCc2c(C)c(F)cc3nc4c(c1c23)Cn1c-4cc2c(c1=O)COC(=O)[C@]2(O)CC1CC1)C1CC1)NC(=O)CCCC#Cc1cnc(S(C)(=O)=O)nc1]

49. (canceled)

50. A conjugate of a linker and a drug, which has the following structure: 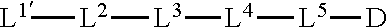
wherein, L^{1′}, L², L³, L⁴, and L⁵ have the definitions as described in claim 14, and D represents a bioactive molecular fragment, preferably a molecular fragment with anti-tumor biological activity,
provided that:
Condition I:
the following hydrophilic moiety is inserted between the fragments as defined above, preferably between L^{1′} and L², or between L² and L³, or is inserted by replacing L²:
hydrophilic moiety: 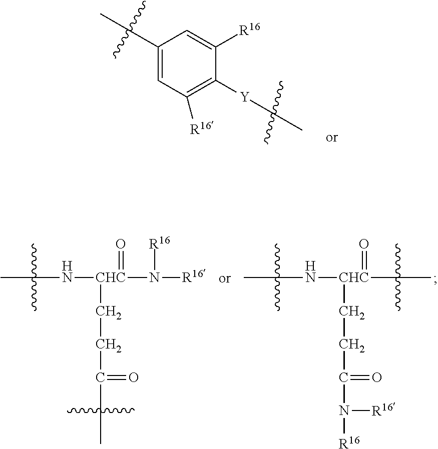
or Condition II:
L^{1′} is a sulfhydryl-reactive group contained in maleimide group or a substituted maleimide group;
the following steric hindrance moiety is inserted between L^{1′} and L², or between L² and L³, or is inserted by replacing L²:
Steric hindrance moiety: 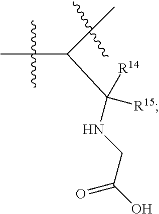
or Condition III:
L^{1′} is a sulfhydryl-reactive group contained in Hal-Het-;
L⁴-L⁵ is: —NR—C(R^{z2}R^{z3})-T^{L}-;
wherein:
R^{z1} is H or C₁₋₄ alkyl;
R^{z2} and R^{z3} are the same or different, and are independently selected from H or C₁₋₄ alkyl;
T^{L} has the definition as described above;
or Condition IV:
L^{1′}-L² has the following structure: 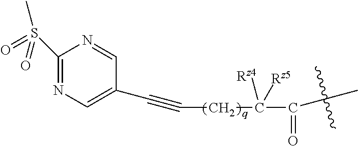
q is an integer of 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
further preferably, R^{z4} and R^{z5} together form a C₃₋₆ cycloalkyl;
L^{1′}-L² is most preferably selected from: 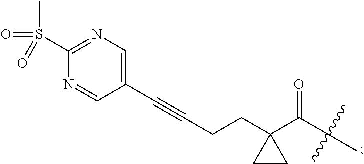 [SMILES: *C(=O)C1(CCC#Cc2cnc(S(C)(=O)=O)nc2)CC1]
particularly, L^{1′} is a sulfhydryl-reactive group contained in maleimide group, a substituted maleimide group or Hal-Het-;
the following hydrophilic moiety is inserted between L¹ and L², or between L² and L³, or is inserted by replacing L²:
hydrophilic moiety: 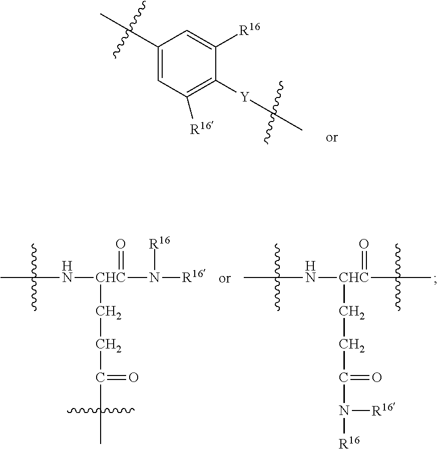
particularly, L^{1′} is a sulfhydryl-reactive group contained in maleimide group or a substituted maleimide group;
the following steric hindrance moiety is inserted between L¹ and L², or between L² and L³, or is inserted by replacing L²:
steric hindrance moiety: 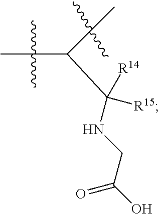
particularly, L^{1′} is a sulfhydryl-reactive group contained in Hal-Het-;
L⁴-L⁵ is: —NR^{z1}—C(R^{z2}R^{z3})—O—(CH₂)ₘ—C(R^{z4}R^{z5})—CO—;
wherein:
R^{z1} is H or C₁₋₄ alkyl;
m is an integer of 0 to 4;
R^{z2} and R^{z3} are the same or different and are independently selected from H or C₁₋₄ alkyl;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl;
particularly, L^{1′}-L² has the following structure: 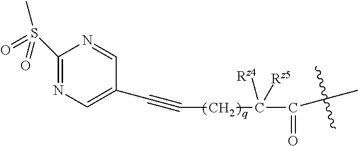
q is an integer of 0 to 10, preferably 1, 2, 3, 4, 5, 6, 7 or 8;
R^{z4} and R^{z5} are the same or different and are independently selected from the group consisting of H, C₁₋₄ alkyl, C₃₋₆ cycloalkyl, C₃₋₆ cycloalkyl-C₁₋₄ alkyl, or R^{z4} and R^{z5} together form a C₃₋₆ cycloalkyl, wherein at least one of R^{z4} and R^{z5} is not H;
further preferably, R^{z4} and R^{z5} together form a C₃₋₆ cycloalkyl;
L^{1′}-L² is most preferably selected from:

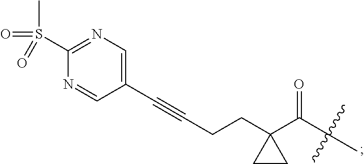 [SMILES: *C(=O)C1(CCC#Cc2cnc(S(C)(=O)=O)nc2)CC1]

51-54. (canceled)

55. A pharmaceutical composition, wherein the pharmaceutical composition comprises the compound represented by Formula (GH), or a stereoisomer, racemate, tautomer, isotopologue, isotope-labeled compound, nitroxide or pharmaceutically acceptable salt thereof according to claim 35;
preferably, the compound represented by the Formula (GH), or a stereoisomer, racemate, tautomer, isotopologue, isotope-labeled compound, nitroxide or pharmaceutically acceptable salt thereof in the pharmaceutical composition is present in a therapeutically effective amount.

56. A pharmaceutical composition, wherein the pharmaceutical composition comprises the conjugate represented by Formula (D), or a stereoisomer, racemate, tautomer, isotopologue, isotope-labeled compound, nitroxide or pharmaceutically acceptable salt thereof according to claim 43;
preferably, the conjugate represented by the Formula (D), or a stereoisomer, racemate, tautomer, isotopologue, isotope-labeled compound, nitroxide or pharmaceutically acceptable salt thereof in the pharmaceutical composition is present in a therapeutically effective amount.

57. A method for preventing and/or treating a disease or disorder, the method comprising administering to a patient therapeutically effective amount of the conjugate represented by of Formula (C) according to claim 1, or a stereoisomer, racemate, tautomer, isotopologue, isotope-labeled compound, nitroxide or pharmaceutically acceptable salt thereof;
preferably, the disease or disorder can be selected from a tumor, such as a solid tumor or a hematological cancer;
preferably, the solid tumor is selected from malignancies of various organ systems, such as sarcomas, adenocarcinomas, blastomas, and carcinomas, such as those affecting liver, lung, breast, lymph, biliary-intestinal (e.g., colon), genitourinary tract (e.g., kidney, urothelial cell), prostate and pharynx. adenocarcinomas comprise, for example, malignancies of most colon cancers, rectal cancers, renal cell carcinomas, liver cancers, small cell lung cancers, non-small cell lung cancers, small intestine cancers and esophageal cancers;
the hematological cancer is selected from the group consisting of leukemia, lymphoma, and malignant lymphoproliferative disorders affecting blood, bone marrow and lymphatic system.

58. A method for preventing and/or treating a disease or disorder, the method comprising administering to a patient therapeutically effective amount of the conjugate represented by of Formula (D) according to claim 43, or a stereoisomer, racemate, tautomer, isotopologue, isotope-labeled compound, nitroxide or pharmaceutically acceptable salt thereof;
preferably, the disease or disorder can be selected from a tumor, such as a solid tumor or a hematological cancer;
preferably, the solid tumor is selected from malignancies of various organ systems, such as sarcomas, adenocarcinomas, blastomas, and carcinomas, such as those affecting liver, lung, breast, lymph, biliary-intestinal (e.g., colon), genitourinary tract (e.g., kidney, urothelial cell), prostate and pharynx. adenocarcinomas comprise, for example, malignancies of most colon cancers, rectal cancers, renal cell carcinomas, liver cancers, small cell lung cancers, non-small cell lung cancers, small intestine cancers and esophageal cancers;
the hematological cancer is selected from the group consisting of leukemia, lymphoma, and malignant lymphoproliferative disorders affecting blood, bone marrow and lymphatic system.
